(19) United States
(12) Patent Application Publication
(10) Pub. No.: US 2023/0078029 A1
(43) Pub. Date: Mar. 16, 2023

(54) LUMINESCENCE DEVICE AND AMINE COMPOUND FOR LUMINESCENCE DEVICE

(71) Applicant: Samsung Display Co., Ltd., Yongin-si (KR)

(72) Inventors: Takuya UNO, Yokohama (JP); Ichinori TAKADA, Yokohama (JP)

(21) Appl. No.: 17/449,885

(22) Filed: Oct. 4, 2021

(30) Foreign Application Priority Data
Jan. 28, 2021 (KR) 10-2021-0012067

(51) Int. Cl. H01L 51/00; C07D 307/91; C09K 11/06; C07D 409/12; C07D 409/14

(52) U.S. Cl. CPC H01L 51/0061; C07D 307/91; C09K 11/06; H01L 51/5088; C07D 409/12; C07D 409/14; H01L 51/006

(57) ABSTRACT

A luminescence device of an embodiment includes a first electrode, a hole transport region disposed on the first electrode, an emission layer disposed on the hole transport region, an electron transport region disposed on the emission layer, and a second electrode disposed on the electron transport region, wherein the hole transport region includes an amine compound represented by Formula 1, thereby showing high efficiency and long life span.

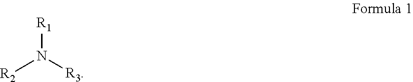

### CROSS-REFERENCE TO RELATED APPLICATION

This application claims priority to and the benefit of Korean Patent Application No. 10-2021-0012067, filed on Jan. 28, 2021, in the Korean Intellectual Property Office, the entire content of which is hereby incorporated by reference.

### BACKGROUND

### 1. Field

One or more aspects of embodiments of the present disclosure relate to a luminescence device and an amine compound for a luminescence device.

### 2. Description of Related Art

Recently, luminescence displays are being activity developed as image displays. A luminescence display is different from a liquid crystal display, and is so-called a self-luminescent display, in which holes and electrons injected from a first electrode and a second electrode recombine in an emission layer so that a light-emitting material including in the emission layer emits light to achieve display.

In the application of a luminescence device to a display, a decreased driving voltage, increased emission efficiency, and increased life span of the luminescence device are desired, and continuous development of materials for a luminescence device capable of stably achieving the requirements is desired.

### SUMMARY

One or more aspects of embodiments of the present disclosure are directed toward a luminescence device and an amine compound for a luminescence device, and for example, a luminescence device having high efficiency and an amine compound included in a hole transport region of a luminescence device.

One or more embodiments of the present disclosure provide an amine compound represented by Formula 1:

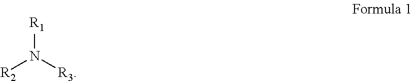

In Formula 1, R₁ is represented by Formula 2-1, and R₂ and R₃ are each independently represented by Formula 2-2 or Formula 2-3.

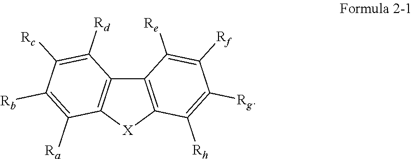

In Formula 2-1, X may be O or S, Rₐ to R_{d} may each independently be a hydrogen atom, a deuterium atom, or a halogen atom, or combined with an adjacent group to form a ring, where any one among (e.g., one selected from among) Rₐ to R_{d} may be a site bonded to nitrogen of Formula 1, any one among Rₑ to Rₕ may be a substituted or unsubstituted alkyl group of 1 to 20 carbon atoms, or a substituted or unsubstituted aryl group of 6 to 30 ring-forming carbon atoms, and the remainder are each independently a hydrogen atom, a deuterium atom, or a halogen atom, or combined with an adjacent group to form a ring, where a fluorenyl group is excluded, and when R_{d} is bonded to the nitrogen of Formula 1, Rₑ may be a hydrogen atom or a deuterium atom, or combined with adjacent R_{f} to form a ring.

*-(L₁)ₘ-Ar₁. Formula 2-2

In Formula 2-2, L₁ may be a substituted or unsubstituted arylene group of 6 to 30 ring-forming carbon atoms, Ar₁ may be a substituted or unsubstituted aryl group of 6 to 30 ring-forming carbon atoms, where a fluorenyl group is excluded, “m” may be an integer of 0 to 3, and when R₂ and R₃ in Formula 1 are both represented by Formula 2-2 at the same time (e.g., simultaneously), Ar₁ is not a 1-naphthyl group.

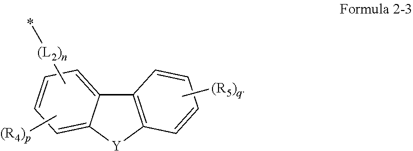

In Formula 2-3, Y may be O or S, L₂ may be a substituted or unsubstituted arylene group of 6 to 30 ring-forming carbon atoms, R₄ may be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring, R₅ may be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring, “n” and “p” may each independently be an integer of 0 to 3, “q” may be an integer of 0 to 4, where Formula 2-1 and Formula 2-3 are not the same (e.g., are different groups), when Rₐ of Formula 2-1 is bonded to the nitrogen of Formula 1, and Rₕ is an alkyl group or an aryl group, at least one selected from among R₂ and R₃ of Formula 1 is represented by Formula 2-3, when X of Formula 2-1 is S, Formula 2-3 is not a 4-dibenzothiophenyl group, and the amine compound represented by Formula 1 includes a compound in which optional hydrogen in a molecule is substituted with deuterium (e.g., at least one hydrogen in the amine compound represented by Formula 1 is optionally substituted with deuterium).

In an embodiment, R₁ of Formula 1 may be represented by Formula 2-1-1, R₂ of Formula 1 may be represented by Formula 2-3, and R₃ of Formula 1 may be represented by Formula 2-2 or Formula 2-3.

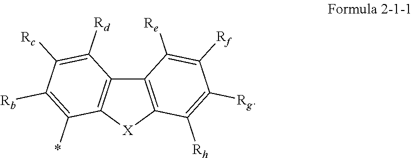

In Formula 2-1-1, X may be O or S, Rₕ may be a substituted or unsubstituted alkyl group of 1 to 20 carbon atoms or a substituted or unsubstituted aryl group of 6 to 30 ring-forming carbon atoms, and R_{b} to R_{g} may each independently be a hydrogen atom, a deuterium atom, or a halogen atom, or combined with an adjacent group to form a ring.

In an embodiment, R₁ of Formula 1 may be represented by Formula 2-1-2, R₂ of Formula 1 may be represented by Formula 2-2, and R₃ of Formula 1 may be represented by Formula 2-2 or Formula 2-3.

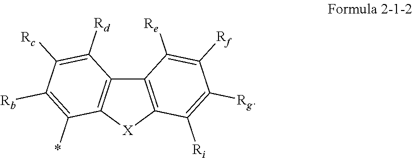

In Formula 2-1-2, X may be O or S, Rᵢ may be a hydrogen atom or a deuterium atom, or combined with an adjacent R_{g} group to form a ring, R_{b} to R_{d} may each independently be a hydrogen atom, a deuterium atom, or a halogen atom, or combined with an adjacent group to form a ring, and any one selected from among Rₑ to R_{g} may be a substituted or unsubstituted alkyl group of 1 to 20 carbon atoms or a substituted or unsubstituted aryl group of 6 to 30 ring-forming carbon atoms, and the remainder may each independently be a hydrogen atom, a deuterium atom, or a halogen atom, or combined with an adjacent group to form a ring, where a fluorenyl group is excluded.

In an embodiment, R₁ of Formula 1 may be represented by Formula 2-1-2, R₂ of Formula 1 may be represented by Formula 2-3, and R₃ of Formula 1 may be represented by Formula 2-2 or Formula 2-3.

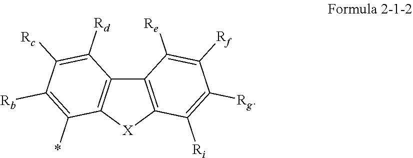

In Formula 2-1-2, X may be O or S, Rᵢ may be a hydrogen atom or a deuterium atom, or combined with an adjacent R_{g} group to form a ring, R_{b} to R_{d} may each independently be a hydrogen atom, a deuterium atom, or a halogen atom, or combined with an adjacent group to form a ring, and any one selected from among Rₑ to R_{g} may be a substituted or unsubstituted alkyl group of 1 to 20 carbon atoms or a substituted or unsubstituted aryl group of 6 to 30 ring-forming carbon atoms, and the remainder may each independently be a hydrogen atom, a deuterium atom, or a halogen atom, or combined with an adjacent group to form a ring, where a fluorenyl group is excluded.

In an embodiment, R₁ of Formula 1 may be represented by Formula 2-1-3, and R₂ and R₃ of Formula 1 may each independently be represented by Formula 2-2 or Formula 2-3.

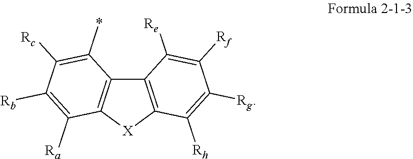

In Formula 2-1-3, X may be O or S, Rₑ may be a hydrogen atom or a deuterium atom, or combined with an adjacent R_{f} group to form a ring, Rₐ to Rₑ may each independently be a hydrogen atom, a deuterium atom, or a halogen atom, or combined with an adjacent group to form a ring, and any one selected from among R_{f} to Rₕ may be a substituted or unsubstituted alkyl group of 1 to 20 carbon atoms or a substituted or unsubstituted aryl group of 6 to 30 ring-forming carbon atoms, and the remainder may each independently be a hydrogen atom, a deuterium atom, or a halogen atom, or combined with an adjacent group to form a ring, where a fluorenyl group is excluded.

In an embodiment, Formula 1 may be represented by Formula 3-1 or Formula 3-2.

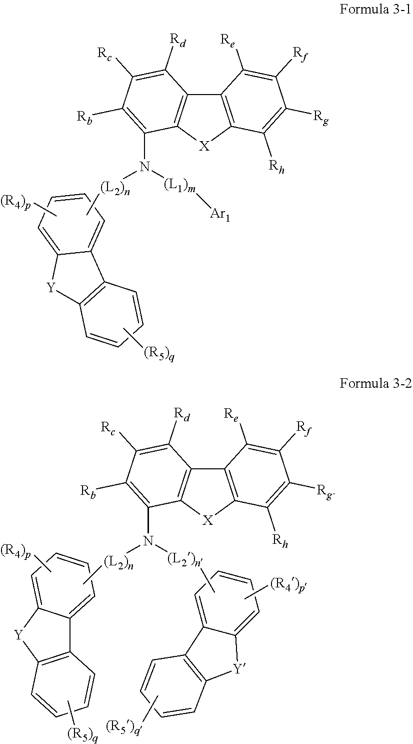

In Formula 3-1 and Formula 3-2, Rₕ may be a substituted or unsubstituted alkyl group of 1 to 20 carbon atoms or a substituted or unsubstituted aryl group of 6 to 30 ring-forming carbon atoms, R_{b} to R_{g} may each independently be a hydrogen atom, a deuterium atom, or a halogen atom, or combined with an adjacent group to form a ring, Y′ may be O or S, L₂′ may be a substituted or unsubstituted arylene group of 6 to 30 ring-forming carbon atoms, R₄′ may be a hydrogen atom, a deuterium atom, a halogen atom, or a substituted or unsubstituted alkyl group of 1 to 20 carbon atoms, and/or combined with an adjacent group to form a ring, R₅′ may be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring, n′ and p′ may each independently be an integer of 0 to 3, q′ may be an integer of 0 to 4, and X, Ar₁, Y, L₁, L₂, R₄, R₅, “m”, “n”, “p” and “q” may each independently be the same as defined in Formula 2-1 to Formula 2-3.

In an embodiment, Formula 1 may be represented by any one selected from among Formula 4-1 to Formula 4-3.

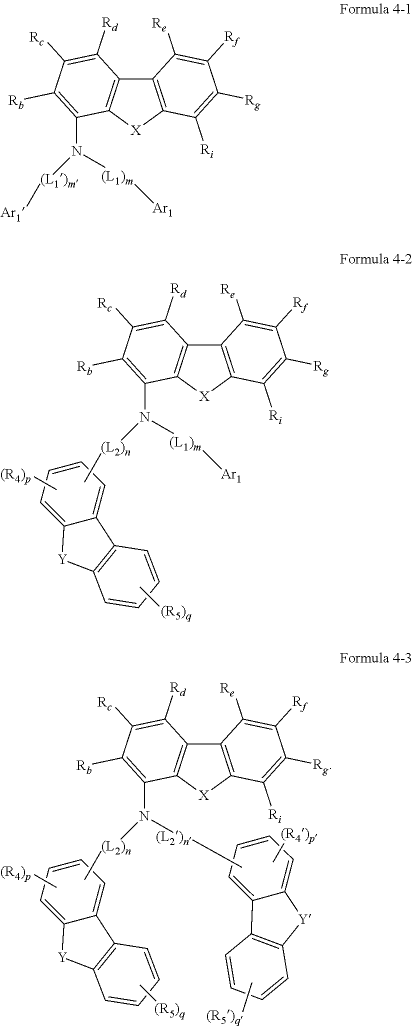

In Formula 4-1 to Formula 4-3, Rᵢ may be a hydrogen atom or a deuterium atom, or combined with an adjacent R_{g} group to form a ring, R_{b} to R_{d} may each independently be a hydrogen atom, a deuterium atom, or a halogen atom, or combined with an adjacent group to form a ring, any one selected from among Rₑ to R_{g} may be a substituted or unsubstituted alkyl group of 1 to 20 carbon atoms or a substituted or unsubstituted aryl group of 6 to 30 ring-forming carbon atoms, and the remainder may each independently be a hydrogen atom, a deuterium atom, or a halogen atom, or combined with an adjacent group to form a ring, where a fluorenyl group is excluded, L₁′ and L₂′ may each independently be a substituted or unsubstituted arylene group of 6 to 30 ring-forming carbon atoms, R₄′ may be a hydrogen atom, a deuterium atom, a halogen atom, or a substituted or unsubstituted alkyl group of 1 to 20 carbon atoms, and/or combined with an adjacent group to form a ring, R₅′ may be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring, n′ and p′ may each independently be an integer of 0 to 3, q′ may be an integer of 0 to 4, and Ar₁ and Ar₁′ may each independently be a substituted or unsubstituted aryl group of 6 to 30 ring-forming carbon atoms, where a fluorenyl group is excluded, m′ may be an integer of 0 to 3, and X, Y, L₁, L₂, R₄, R₅, “m”, “n”, “p” and “q” may each independently be the same as defined in Formula 2-1 to Formula 2-3, where Ar₁ and Ar₁′ in Formula 4-1 are not 1-naphthyl groups at the same time (e.g., simultaneously).

In an embodiment, Formula 1 may be represented by any one selected from among Formula 5-1 to Formula 5-3.

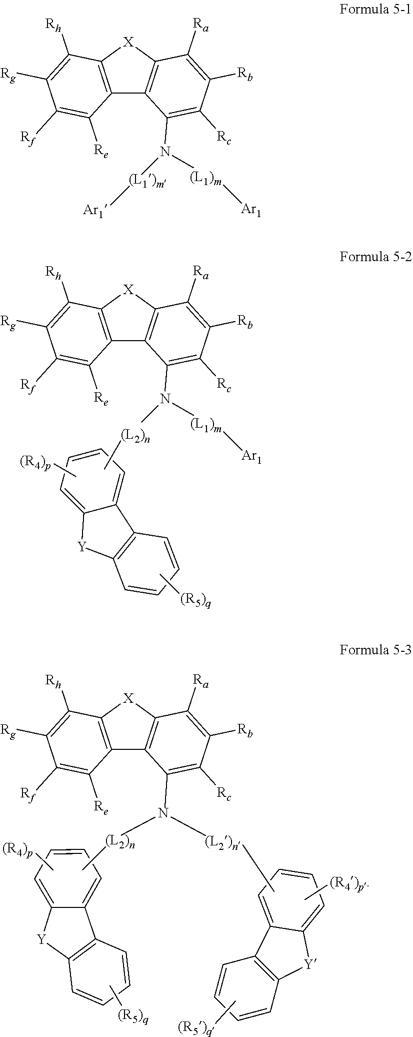

In Formula 5-1 to Formula 5-3, Rₑ may be a hydrogen atom or a deuterium atom, or combined with an adjacent R_{f} group to form a ring, Rₐ to Rₑ may each independently be a hydrogen atom, a deuterium atom, or a halogen atom, or combined with an adjacent group to form a ring, any one selected from among R_{f} to Rₕ may be a substituted or unsubstituted alkyl group of 1 to 20 carbon atoms or a substituted or unsubstituted aryl group of 6 to 30 ring-forming carbon atoms, and the remainder are each independently a hydrogen atom, a deuterium atom, or a halogen atom, or combined with an adjacent group to form a ring, where a fluorenyl group is excluded, L₁′ may be a substituted or unsubstituted arylene group of 6 to 30 ring-forming carbon atoms, Ar₁ and Ar₁′ may each independently be a substituted or unsubstituted aryl group of 6 to 30 ring-forming carbon atoms, where a fluorenyl group is excluded, m′ may be an integer of 0 to 3, Y′ may be O or S, L₂′ may be a substituted or unsubstituted arylene group of 6 to 30 ring-forming carbon atoms, R₄′ may be a hydrogen atom, a deuterium atom, a halogen atom, or a substituted or unsubstituted alkyl group of 1 to 20 carbon atoms, and/or combined with an adjacent group to form a ring, R₅′ may be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring, n′ and p′ may each independently be an integer of 0 to 3, q′ may be an integer of 0 to 4, and X, Y, L₁, L₂, R₄, R₅, “m”, “n”, “p” and “q” may each independently be the same as defined in Formula 2-1 to Formula 2-3, where Ar₁ and Ar₁′ in Formula 5-1 are not 1-naphthyl groups at the same time (e.g., simultaneously).

In an embodiment, L₁ and L₂ of Formula 2-2 and Formula 2-3 may each independently be a direct linkage, a substituted or unsubstituted phenylene group, a substituted or unsubstituted divalent biphenyl group, or a substituted or unsubstituted naphthylenyl group.

In an embodiment, the amine compound represented by Formula 1 may be any one selected from among compounds represented in Compound Group 1 to Compound Group 3.

One or more embodiments of the present disclosure provide a luminescence device including a first electrode, a hole transport region provided on the first electrode, an emission layer provided on the hole transport region, an electron transport region provided on the emission layer, and a second electrode provided on the electron transport region, wherein the hole transport region includes an amine compound according to an embodiment.

In an embodiment, the hole transport region may include a hole injection layer disposed on the first electrode, and a hole transport layer disposed on the hole injection layer, and the hole transport layer or the hole injection layer may include the amine compound of an embodiment.

In an embodiment, the hole transport region may include a hole transport layer disposed on the first electrode, and an electron blocking layer disposed on the hole transport layer, and the electron blocking layer may include the amine compound of an embodiment.

### BRIEF DESCRIPTION OF THE DRAWINGS

The accompanying drawings are included to provide a further understanding of the present disclosure, and are incorporated in and constitute a part of this specification. The drawings illustrate embodiments of the present disclosure and, together with the description, serve to explain principles of the present disclosure. In the drawings:

FIG. 1 is a plan view showing a display apparatus according to an embodiment of the present disclosure;

FIG. 2 is a cross-sectional view showing a display apparatus according to an embodiment of the present disclosure;

FIG. 3 is a cross-sectional view schematically showing a luminescence device according to an embodiment of the present disclosure;

FIG. 4 is a cross-sectional view schematically showing a luminescence device according to an embodiment of the present disclosure;

FIG. 5 is a cross-sectional view schematically showing a luminescence device according to an embodiment of the present disclosure;

FIG. 6 is a cross-sectional view schematically showing a luminescence device according to an embodiment of the present disclosure;

FIG. 7 is a cross-sectional view showing a display apparatus according to an embodiment of the present disclosure; and

FIG. 8 is a cross-sectional view showing a display apparatus according to an embodiment of the present disclosure.

### DETAILED DESCRIPTION

The present disclosure may have one or more suitable modifications and may be embodied in different forms, and embodiments will be explained in more detail with reference to the accompanying drawings. The present disclosure may, however, be embodied in different forms and should not be construed as being limited to the embodiments set forth herein. Rather, all modifications, equivalents, and substituents that are included in the spirit and technical scope of the present disclosure should be included in the present disclosure.

Like reference numerals refer to like elements throughout, and duplicative descriptions thereof may not be provided. In the drawings, the dimensions of structures may be exaggerated for clarity of illustration. It will be understood that, although the terms first, second, etc. may be used herein to describe various elements, these elements should not be limited by these terms. These terms are only used to distinguish one element from another element. Thus, a first element could be alternatively termed a second element without departing from the teachings of the present disclosure. Similarly, a second element could be alternatively termed a first element. As used herein, singular forms such as “a,” “an,” and “the” are intended to include the plural forms as well, unless the context clearly indicates otherwise.

In the description, it will be further understood that the terms “includes,” “including,” “comprises,” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof.

In the description, when a layer, a film, a region, a plate, etc. is referred to as being “on” or “above” another part, it can be “directly on” the other part, or intervening layers may also be present. In contrast, when a layer, a film, a region, a plate, etc. is referred to as being “under” or “below” another part, it can be “directly under” the other part, or intervening layers may also be present. Also, when an element is referred to as being disposed “on” another element, it can be disposed under the other element.

As used herein, the terms “use,” “using,” and “used” may be considered synonymous with the terms “utilize,” “utilizing,” and “utilized,” respectively. As used herein, expressions such as “at least one of,” “one of,” and “selected from,” when preceding a list of elements, modify the entire list of elements and do not modify the individual elements of the list.

As used herein, the term “and/or” includes any and all combinations of one or more of the associated listed items. Further, the use of “may” when describing embodiments of the present disclosure refers to “one or more embodiments of the present disclosure”.

Hereinafter, embodiments of the present disclosure will be explained by referring to the drawings.

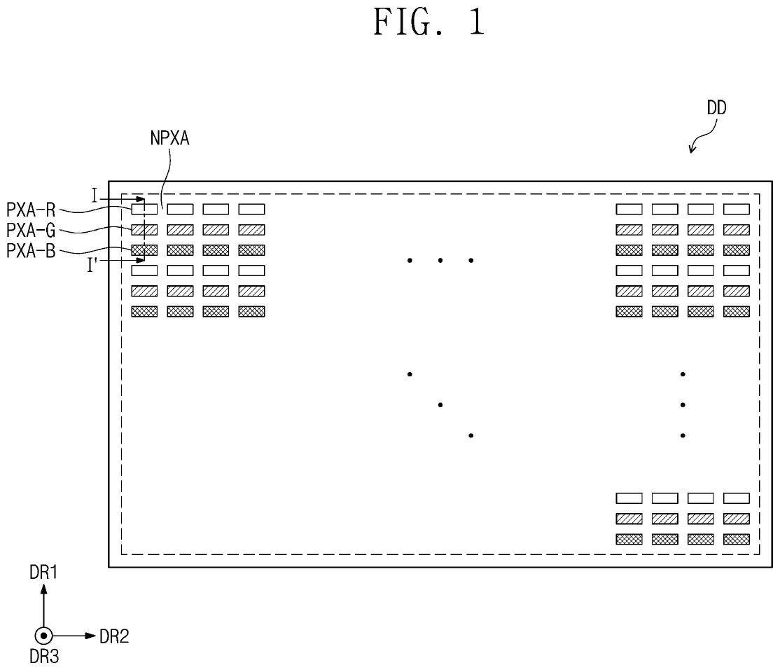

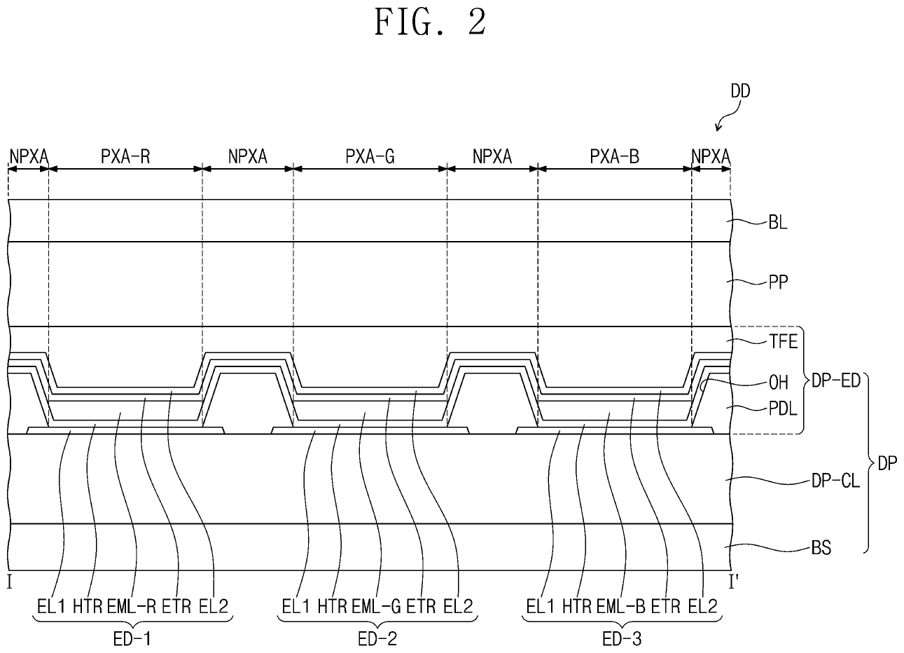

FIG. 1 is a plan view showing an embodiment of a display apparatus DD. FIG. 2 is a cross-sectional view of a display apparatus DD of an embodiment. FIG. 2 is a cross-sectional view showing a part corresponding to line I-I′.

The display apparatus DD may include a display panel DP and an optical layer PP disposed on the display panel DP. The display panel DP includes luminescence devices ED-1, ED-2 and ED-3. The display apparatus DD may include multiple luminescence devices ED-1, ED-2 and/or ED-3. The optical layer PP may be disposed on the display panel DP and may control or reduce reflection of external light by the display panel DP. The optical layer PP may include, for example, a polarization layer or a color filter layer. In some embodiments, different from the drawings, the optical layer PP may not be provided in the display apparatus DD of an embodiment.

On the optical layer PP, a base substrate BL may be disposed. The base substrate BL may be a member providing a base surface where the optical layer PP is disposed. The base substrate BL may be a glass substrate, a metal substrate, a plastic substrate, etc. However, embodiments of the present disclosure are not limited thereto, and the base substrate BL may be an inorganic layer, an organic layer or a composite material layer. In some embodiments, the base substrate BL may not be provided.

The display apparatus DD according to an embodiment may further include a plugging layer. The plugging layer may be disposed between a display device layer DP-ED and a base substrate BL. The plugging layer may be an organic layer. The plugging layer may include at least one selected from among an acrylic resin, a silicon-based resin and an epoxy-based resin.

The display panel DP may include a base layer BS, a circuit layer DP-CL provided on the base layer BS and a display device layer DP-ED. The display device layer DP-ED may include a pixel definition layer PDL, luminescence devices ED-1, ED-2 and ED-3 disposed in the pixel definition layer PDL, and an encapsulating layer TFE disposed on the luminescence devices ED-1, ED-2 and ED-3.

The base layer BS may be a member providing a base surface where the display device layer DP-ED is disposed. The base layer BS may be a glass substrate, a metal substrate, a plastic substrate, etc. However, embodiments of the present disclosure are not limited thereto, and the base layer BS may be an inorganic layer, an organic layer or a composite material layer.

In an embodiment, the circuit layer DP-CL is disposed on the base layer BS, and the circuit layer DP-CL may include multiple transistors. Each of the transistors may include a control electrode, an input electrode, and an output electrode. For example, the circuit layer DP-CL may include switching transistors and driving transistors for driving the luminescence devices ED-1, ED-2 and ED-3 of the display device layer DP-ED.

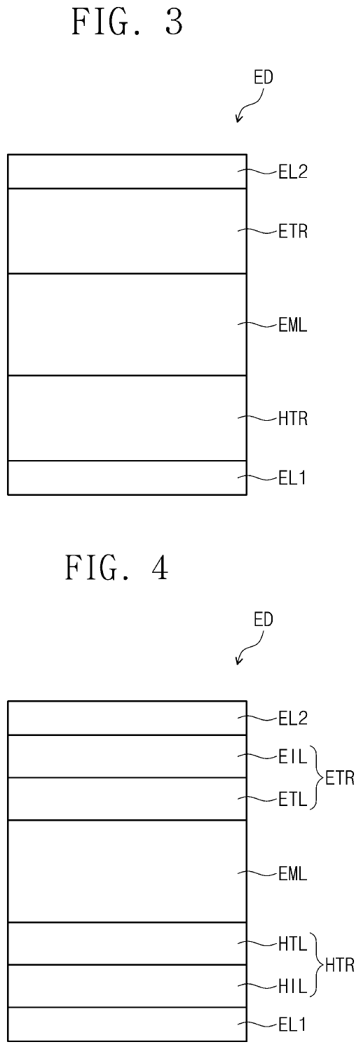

Each of the luminescence devices ED-1, ED-2 and ED-3 may have the structures of any of the luminescence devices ED of embodiments according to FIG. 3 to FIG. 6, which will be explained later. Each of the luminescence devices ED-1, ED-2 and ED-3 may include a first electrode EL1, a hole transport region HTR, emission layers EML-R, EML-G and EML-B, an electron transport region ETR and a second electrode EL2.

In FIG. 2, shown is an embodiment where the emission layers EML-R, EML-G and EML-B of luminescence devices ED-1, ED-2 and ED-3 are disposed in respective opening portions OH defined in a pixel definition layer PDL, and a hole transport region HTR, an electron transport region ETR and a second electrode EL2 are provided as common layers in all luminescence devices ED-1, ED-2 and ED-3. However, embodiments of the present disclosure are not limited thereto. In some embodiments, the hole transport region HTR and the electron transport region ETR may be patterned and provided in separate opening portions OH defined in the pixel definition layer PDL. For example, in an embodiment, the hole transport region HTR, the emission layers EML-R, EML-G and EML-B, and the electron transport region ETR of the luminescence devices ED-1, ED-2 and ED-3 may be patterned and provided by an ink jet printing method.

An encapsulating layer TFE may cover the luminescence devices ED-1, ED-2 and ED-3. The encapsulating layer TFE may encapsulate the display device layer DP-ED. The encapsulating layer TFE may be a thin film encapsulating layer. The encapsulating layer TFE may be one layer or a stacked layer of multiple layers. The encapsulating layer TFE includes at least one insulating layer. The encapsulating layer TFE according to an embodiment may include at least one inorganic layer (hereinafter, encapsulating inorganic layer). In some embodiments, the encapsulating layer TFE according to an embodiment may include at least one organic layer (hereinafter, encapsulating organic layer) and at least one encapsulating inorganic layer.

The encapsulating inorganic layer protects the display device layer DP-ED from moisture/oxygen, and the encapsulating organic layer protects the display device layer DP-ED from foreign materials (such as dust particles). The encapsulating inorganic layer may include silicon nitride, silicon oxy nitride, silicon oxide, titanium oxide, and/or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be disposed on the second electrode EL2 and may be disposed while filling the opening portion OH.

Referring to FIG. 1 and FIG. 2, the display apparatus DD may include a non-luminous area NPXA and luminous areas PXA-R, PXA-G and PXA-B. The luminous areas PXA-R, PXA-G and PXA-B may be areas emitting light produced from the luminescence devices ED-1, ED-2 and ED-3, respectively. The luminous areas PXA-R, PXA-G and PXA-B may be separated from each other on a plane.

The luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. In some embodiments, each of the luminous areas PXA-R, PXA-G and PXA-B may correspond to a pixel. The pixel definition layer PDL may divide the luminescence devices ED-1, ED-2 and ED-3. The emission layers EML-R, EML-G and EML-B of the luminescence devices ED-1, ED-2 and ED-3 may be disposed and divided in the opening portions OH defined in the pixel definition layer PDL.

The luminous areas PXA-R, PXA-G and PXA-B may be divided into multiple groups according to the color of light produced from the luminescence devices ED-1, ED-2 and ED-3. In the display apparatus DD of an embodiment, shown in FIG. 1 and FIG. 2, three luminous areas PXA-R, PXA-G and PXA-B to respectively emit red light, green light and blue light are illustrated as an embodiment. For example, the display apparatus DD of an embodiment may include a red luminous area PXA-R, a green luminous area PXA-G and a blue luminous area PXA-B, which are separated from each other.

In the display apparatus DD according to an embodiment, multiple luminescence devices ED-1, ED-2 and ED-3 may be to emit light having different wavelength regions. For example, in an embodiment, the display apparatus DD may include a first luminescence device ED-1 to emit red light, a second luminescence device ED-2 to emit green light, and a third luminescence device ED-3 to emit blue light. For example, the red luminous area PXA-R, the green luminous area PXA-G, and the blue luminous area PXA-B of the display apparatus DD may respectively correspond to the first luminescence device ED-1, the second luminescence device ED-2, and the third luminescence device ED-3.

However, embodiments of the present disclosure are not limited thereto, and the first to third luminescence devices ED-1, ED-2 and ED-3 may be to emit light in substantially the same wavelength region, or at least one thereof may be to emit light in a different wavelength region. For example, all the first to third luminescence devices ED-1, ED-2 and ED-3 may be to emit blue light.

The luminous areas PXA-R, PXA-G and PXA-B in the display apparatus DD according to an embodiment may be arranged in a stripe shape. Referring to FIG. 1, multiple red luminous areas PXA-R may be arranged with each other along a second direction axis DR2, multiple green luminous areas PXA-G may be arranged with each other along the second direction axis DR2, and multiple blue luminous areas PXA-B may be arranged with each other along the second direction axis DR2. In some embodiments, the red luminous area PXA-R, the green luminous area PXA-G and the blue luminous area PXA-B may be arranged with each other by turns along a first direction axis DR1.

In FIG. 1 and FIG. 2, the areas of the luminous areas PXA-R, PXA-G and PXA-B are shown as being similar, but embodiments of the present disclosure are not limited thereto. The areas of the luminous areas PXA-R, PXA-G and PXA-B may be selected to be different from each other according to the wavelength region of light emitted. The areas of the luminous areas PXA-R, PXA-G and PXA-B may refer to areas on a plane defined by the first direction axis DR1 and the second direction axis DR2 (e.g., areas in a plan view).

The arrangement type or pattern of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in various suitable combinations according to the properties of display quality required for the display apparatus DD. For example, the arrangement pattern of the luminous areas PXA-R, PXA-G and PXA-B may be a PENTILE® arrangement pattern, or a diamond arrangement pattern.

In some embodiments, the areas of the luminous areas PXA-R, PXA-G and PXA-B may be different from each other. For example, in an embodiment, the area of the green luminous area PXA-G may be smaller than the area of the blue luminous area PXA-B, but embodiments of the present disclosure are not limited thereto.

Hereinafter, FIG. 3 to FIG. 6 are cross-sectional views schematically showing luminescence devices according to embodiments. The luminescence device ED according to an embodiment may include a first electrode EL1, a hole transport region HTR, an emission layer EML, and a second electrode EL2 stacked in order.

The luminescence device ED of an embodiment may include an amine compound, which will be explained later, in the hole transport region HTR disposed between the first electrode EL1 and the second electrode EL2. However, embodiments of the present disclosure are not limited thereto, and the luminescence device ED of an embodiment may include a compound, which will be explained later, in an emission layer EML or an electron transport region ETR, which correspond to multiple functional layers disposed between the first electrode EL1 and the second electrode EL2, or in a capping layer CPL disposed on the second electrode EL2 in addition to the hole transport region HTR.

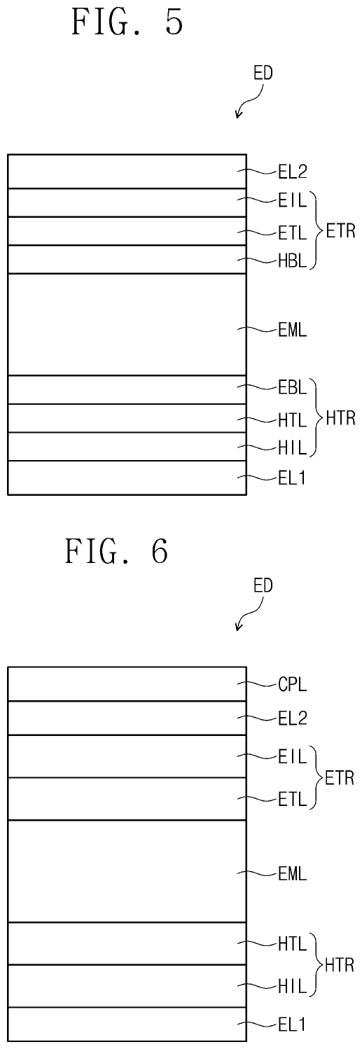

Compared with FIG. 3, FIG. 4 shows the cross-sectional view of a luminescence device ED of an embodiment, wherein a hole transport region HTR includes a hole injection layer HIL and a hole transport layer HTL, and an electron transport region ETR includes an electron injection layer EIL and an electron transport layer ETL. Compared with FIG. 3, FIG. 5 shows the cross-sectional view of a luminescence device ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 4, FIG. 6 shows the cross-sectional view of a luminescence device ED of an embodiment, including a capping layer CPL disposed on the second electrode EL2.

The first electrode EL1 has conductivity. The first electrode EL1 may be formed utilizing a metal alloy or a conductive compound. The first electrode EL1 may be an anode or a cathode. However, embodiments of the present disclosure are not limited thereto. In some embodiments, the first electrode EL1 may be a pixel electrode. The first electrode EL1 may be a transmissive electrode, a transflective electrode, or a reflective electrode. When the first electrode EL1 is a transmissive electrode, the first electrode EL1 may include a transparent metal oxide (such as indium tin oxide (ITO), indium zinc oxide (IZO), zinc oxide (ZnO), and/or indium tin zinc oxide (ITZO)). When the first electrode EL1 is a transflective electrode or a reflective electrode, the first electrode EL1 may include silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), LiF/Ca, LiF/Al, molybdenum (Mo), titanium (Ti), one or more compounds thereof, or one or more mixtures thereof (for example, a mixture of Ag and Mg). In some embodiments, the first electrode EL1 may have a structure of multiple layers including a reflective layer or a transflective layer formed utilizing the above materials, and a transmissive conductive layer formed utilizing ITO, IZO, ZnO, or ITZO. For example, the first electrode EL1 may include a three-layer structure of ITO/Ag/ITO. However, embodiments of the present disclosure are not limited thereto. The thickness of the first electrode EL1 may be about 700 Å to about 10,000 Å. For example, the thickness of the first electrode EL1 may be about 1,000 Å to about 3,000 Å.

The hole transport region HTR is provided on the first electrode EL1. The hole transport region HTR may include at least one selected from among a hole injection layer HIL, a hole transport layer HTL, a hole buffer layer, and an electron blocking layer EBL. The thickness of the hole transport region HTR may be, for example, about 50 Å to about 15,000 Å.

The hole transport region HTR may have a single layer formed utilizing a single material, a single layer formed utilizing multiple different materials, or a multilayer structure including multiple layers formed utilizing multiple different materials.

For example, the hole transport region HTR may have the structure of a single layer of a hole injection layer HIL or a hole transport layer HTL, and may have a structure of a single layer formed utilizing a hole injection material and a hole transport material. In some embodiments, the hole transport region HTR may have a structure of a single layer formed utilizing multiple different materials, or a structure stacked from the first electrode EL1 of hole injection layer HIL/hole transport layer HTL, hole injection layer HIL/hole transport layer HTL/hole buffer layer, hole injection layer HIL/hole buffer layer, hole transport layer HTL/hole buffer layer, or hole injection layer HIL/hole transport layer HTL/electron blocking layer EBL, without limitation.

The hole transport region HTR of the luminescence device ED of an embodiment may include an amine compound according to embodiments of the present disclosure.

In the description, the term “substituted or unsubstituted” refers to being unsubstituted, or substituted with at least one substituent selected from the group consisting of a deuterium atom, a halogen atom, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkoxy group, a hydrocarbon ring group, an aryl group, and a heterocyclic group. Each of the exemplified substituents may be further substituted or unsubstituted. For example, a biphenyl group may be interpreted as a named aryl group, or as a phenyl group substituted with a phenyl group.

In the description, the halogen atom may be a fluorine atom, a chlorine atom, a bromine atom or an iodine atom.

In the description, the alkyl group may be a linear, branched or cyclic alkyl group. The carbon number of the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the description, the term “alkenyl group” may refer to a hydrocarbon group including one or more carbon double bonds in the middle or at the terminal of an alkyl group of 2 or more carbon atoms. The alkenyl group may be a linear chain or a branched chain. The carbon number is not specifically limited but may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

In the description, the term “alkynyl group” may refer to a hydrocarbon group including one or more carbon triple bonds in the middle or at the terminal of an alkyl group of 2 or more carbon atoms. The alkynyl group may be a linear chain or a branched chain. The carbon number is not specifically limited but may be 2 to 30, 2 to 20, or 2 to 10. Examples of the alkynyl group may include an ethynyl group, a propynyl group, etc., without limitation.

In the description, the term “hydrocarbon ring group” refers to an optional functional group or substituent derived from an aliphatic hydrocarbon ring, or an optional functional group or substituent derived from an aromatic hydrocarbon ring. The carbon number for forming rings of the hydrocarbon ring group may be 5 to 60, 5 to 30, or 5 to 20.

In the description, the term “aryl group” refers to an optional functional group or substituent derived from an aromatic hydrocarbon ring. The aryl group may be a monocyclic aryl group or a polycyclic aryl group. The carbon number for forming rings in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

In the description, the fluorenyl group may be substituted (e.g., at the 9H position), and two substituents may be combined with each other to form a spiro structure. Examples of a substituted fluorenyl group are as follows. However, embodiments of the present disclosure are not limited thereto.

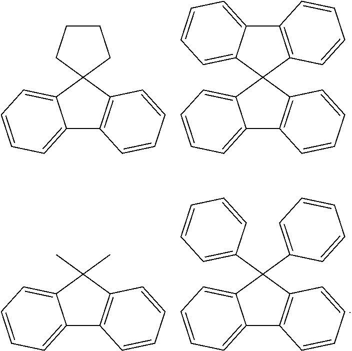

In the description, the term “heterocyclic group” may refer to an optional functional group or substituent derived from a ring including one or more among boron (B), oxygen (O), nitrogen (N), phosphorus (P), silicon (Si) and sulfur (S) as heteroatoms. The heterocyclic group may be an aliphatic heterocyclic group or an aromatic heterocyclic group. The aromatic heterocyclic group may be a heteroaryl group. The aliphatic heterocyclic group and the aromatic heterocyclic group may be a monocycle or a polycycle.

In the description, the heterocyclic group may include one or more among B, O, N, P, Si and S as heteroatoms. When the heterocyclic group includes two or more heteroatoms, the two or more heteroatoms may be the same or different. The heterocyclic group may be a monocyclic heterocyclic group or a polycyclic heterocyclic group and has the concept including a heteroaryl group. The carbon number for forming rings of the heterocyclic group may be 2 to 30, 2 to 20, or 2 to 10.

In the description, the aliphatic heterocyclic group may include one or more among B, O, N, P, Si and S as heteroatoms. The carbon number for forming rings of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

In the description, the heteroaryl group may include one or more among B, O, N, P, Si and S as heteroatoms. When the heteroaryl group includes two or more heteroatoms, two or more heteroatoms may be the same or different. The heteroaryl group may be a monocyclic heterocyclic group or polycyclic heterocyclic group. The carbon number for forming rings of the heteroaryl group may be 2 to 30, 2 to 20, or 2 to 10. Examples of the heteroaryl group may include thiophene, furan, pyrrole, imidazole, triazole, pyridine, bipyridine, pyrimidine, triazine, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the description, the carbon number of the amine group is not specifically limited, but may be 1 to 30. The amine group may include an alkyl amine group, an aryl amine group, or a heteroaryl amine group. Examples of the amine group may include a methylamine group, a dimethylamine group, a phenylamine group, a diphenylamine group, a naphthylamine group, a 9-methyl-anthracenylamine group, a triphenylamine group, etc., without limitation.

In the description, the explanation on the aryl group may be applied to the arylene group except that the arylene group is a divalent group.

In the description, the explanation on the heteroaryl group may be applied to the heteroarylene group except that the heteroarylene group is a divalent group.

In some embodiments, in the description, “ [symbol] ” refers to a position to be connected.

The amine compound according to embodiments of the present disclosure are represented by Formula 1.

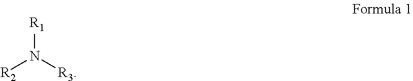

In Formula 1, R₁ is represented by Formula 2-1, and R₂ and R₃ are each independently represented by Formula 2-2 or Formula 2-3.

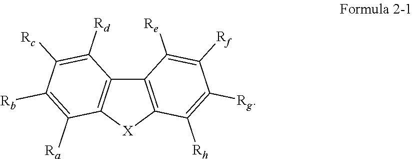

In Formula 2-1, X may be O or S.

In Formula 2-1, Rₐ to R_{d} may each independently be a hydrogen atom, a deuterium atom, or a halogen atom, or combined with an adjacent group to form a ring, where any one selected from among Rₐ to R_{d} may be a site bonded to nitrogen of Formula 1.

In Formula 2-1, any one selected from among Rₑ to Rₕ may be a substituted or unsubstituted alkyl group of 1 to 20 carbon atoms, or a substituted or unsubstituted aryl group of 6 to 30 ring-forming carbon atoms, and the remainder may each independently be a hydrogen atom, a deuterium atom, or a halogen atom, or combined with an adjacent group to form a ring, where a fluorenyl group is excluded.

In Formula 2-1, when R_{d} is bonded to the nitrogen of Formula 1, Rₑ may be a hydrogen atom or a deuterium atom, or combined with adjacent R_{f} to form a ring.

*-(L₁)ₘ-Ar₁. Formula 2-2

In Formula 2-2, L₁ may be a substituted or unsubstituted arylene group of 6 to 30 ring-forming carbon atoms.

In Formula 2-2, Ar₁ may be a substituted or unsubstituted aryl group of 6 to 30 ring-forming carbon atoms, where a fluorenyl group is excluded. In some embodiments, when R₂ and R₃ in Formula 1 are represented by Formula 2-2 at the same time (e.g., simultaneously), Ar₁ is not a 1-naphthyl group.

In Formula 2-2, “m” may be an integer of 0 to 3, and when “m” is 2 or more, multiple L₁ groups may each independently be the same or different.

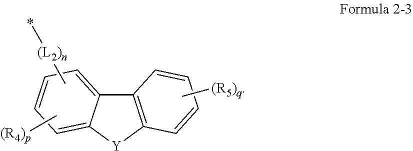

In Formula 2-3, Y may be O or S.

In Formula 2-3, L₂ may be a substituted or unsubstituted arylene group of 6 to 30 ring-forming carbon atoms.

In Formula 2-3, R₄ may be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, and/or combined with an adjacent group to form a ring.

In Formula 2-3, R₅ may be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring.

In Formula 2-3, “n” and “p” may each independently be an integer of 0 to 3. In some embodiments, when “n” is 2 or more, multiple L₂ groups may each independently be the same or different, and when “p” is 2 or more, multiple R₄ groups may each independently be the same or different.

In Formula 2-3, “q” may be an integer of 0 to 4. In some embodiments, when “q” is 2 or more, multiple R₅ groups may each independently be the same or different.

Here, Formula 2-1 and Formula 2-3 are always different.

In some embodiments, when Rₐ of Formula 2-1 is bonded to the nitrogen of Formula 1, and Rₕ is an alkyl group or an aryl group, at least one selected from among R₂ and R₃ of Formula 1 may be represented by Formula 2-3. In this case, when X of Formula 2-1 is S, Formula 2-3 is not a 4-dibenzothiophenyl group.

The amine compound represented by Formula 1 according to an embodiment includes a compound in which optional hydrogen in a molecule is substituted with deuterium.

In an embodiment, in Formula 1, R₁ may be represented by Formula 2-1-1, R₂ may be represented by Formula 2-3, and R₃ may be represented by Formula 2-2 or Formula 2-3.

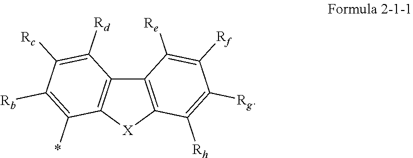

In Formula 2-1-1, X may be O or S, and Rₕ may be a substituted or unsubstituted alkyl group of 1 to 20 carbon atoms or a substituted or unsubstituted aryl group of 6 to 30 ring-forming carbon atoms.

In Formula 2-1-1, R_{b} to R_{g} may each independently be a hydrogen atom, a deuterium atom, or a halogen atom, or combined with an adjacent group to form a ring.

In an embodiment, in Formula 1, R₁ may be represented by Formula 2-1-2, R₂ may be represented by Formula 2-2, and R₃ may be represented by Formula 2-2 or Formula 2-3.

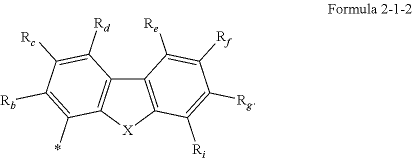

In Formula 2-1-2, X may be O or S, and Rᵢ may be a hydrogen atom or a deuterium atom, or combined with an adjacent R_{g} group to form a ring.

In Formula 2-1-2, R_{b} to R_{d} may each independently be a hydrogen atom, a deuterium atom, or a halogen atom, or combined with an adjacent group to form a ring.

In Formula 2-1-2, any one selected from among Rₑ to R_{g} may be a substituted or unsubstituted alkyl group of 1 to 20 carbon atoms or a substituted or unsubstituted aryl group of 6 to 30 ring-forming carbon atoms, and the remainder may each independently be a hydrogen atom, a deuterium atom, or a halogen atom, or combined with an adjacent group to form a ring, where a fluorenyl group is excluded.

In an embodiment, in Formula 1, R₁ may be represented by Formula 2-1-3, and R₂ and R₃ may be represented by Formula 2-2 or Formula 2-3.

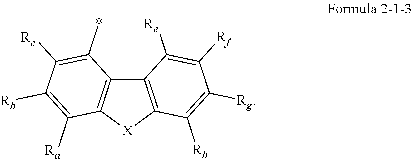

In Formula 2-1-3, X may be O or S, and Rₑ may be a hydrogen atom or a deuterium atom, or combined with an adjacent R_{f} group to form a ring.

In Formula 2-1-3, Rₐ to Rₑ may each independently be a hydrogen atom, a deuterium atom, or a halogen atom, or combined with an adjacent group to form a ring.

In Formula 2-1-3, any one selected from among R_{f} to Rₕ may be a substituted or unsubstituted alkyl group of 1 to 20 carbon atoms or a substituted or unsubstituted aryl group of 6 to 30 ring-forming carbon atoms, and the remainder may each independently be a hydrogen atom, a deuterium atom, or a halogen atom, or combined with an adjacent group to form a ring, where a fluorenyl group is excluded.

In an embodiment, Formula 1 may be represented by Formula 3-1 or Formula 3-2.

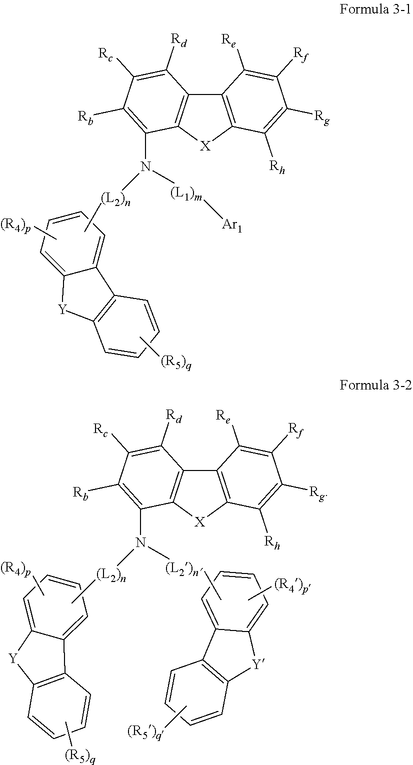

In Formula 3-1 and Formula 3-2, Rₕ may be a substituted or unsubstituted alkyl group of 1 to 20 carbon atoms or a substituted or unsubstituted aryl group of 6 to 30 ring-forming carbon atoms.

In Formula 3-1 and Formula 3-2, R_{b} to R_{g} may each independently be a hydrogen atom, a deuterium atom, or a halogen atom, or combined with an adjacent group to form a ring.

In Formula 3-2, Y′ may be O or S, L₂′ may be a substituted or unsubstituted arylene group of 6 to 30 ring-forming carbon atoms.

In Formula 3-2, R₄′ may be a hydrogen atom, a deuterium atom, a halogen atom, or a substituted or unsubstituted alkyl group of 1 to 20 carbon atoms, and/or combined with an adjacent group to form a ring.

In Formula 3-2, R₅′ may be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, and/or combined with an adjacent group to form a ring.

In Formula 3-2, n′ and p′ may each independently be an integer of 0 to 3. In some embodiments, when n′ is 2 or more, multiple L₂ groups may each independently be the same or different, and when p′ is 2 or more, multiple R₄′ groups may each independently be the same or different.

In Formula 3-2, q′ may be an integer of 0 to 4. In some embodiments, when q′ is 2 or more, multiple R₅′ groups may each independently be the same or different.

In Formula 3-1 and Formula 3-2, X, Ar₁, Y, L₁, L₂, R₄, R₅, “m”, “n”, “p” and “q” may each independently be the same as defined in Formula 2-1 to Formula 2-3.

In an embodiment, Formula 1 may be represented by any one selected from among Formula 4-1 to Formula 4-3.

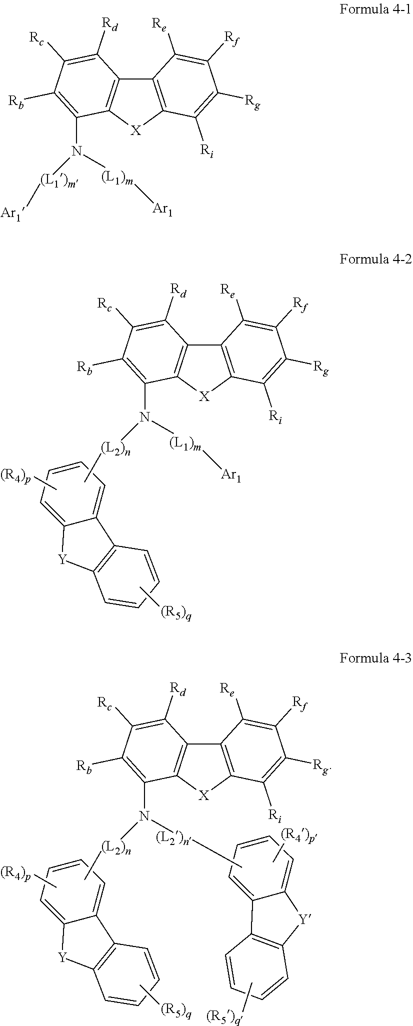

In Formula 4-1 to Formula 4-3, Rᵢ may be a hydrogen atom or a deuterium atom, or combined with an adjacent R_{g} group to form a ring.

In Formula 4-1 to Formula 4-3, R_{b} to R_{d} may each independently be a hydrogen atom, a deuterium atom, or a halogen atom, or combined with an adjacent group to form a ring.

In Formula 4-1 to Formula 4-3, any one selected from among Rₑ to R_{g} may be a substituted or unsubstituted alkyl group of 1 to 20 carbon atoms or a substituted or unsubstituted aryl group of 6 to 30 ring-forming carbon atoms, and the remainder may each independently be a hydrogen atom, a deuterium atom, or a halogen atom, or combined with an adjacent group to form a ring, where a fluorenyl group is excluded.

In Formula 4-1 to Formula 4-3, L₁′ and L₂′ may each independently be a substituted or unsubstituted arylene group of 6 to 30 ring-forming carbon atoms.

In Formula 4-1, Ar₁′ may be a substituted or unsubstituted aryl group of 6 to 30 ring-forming carbon atoms, where a fluorenyl group is excluded.

In Formula 4-1, m′ may be an integer of 0 to 3. In some embodiments, when m′ is 2 or more, multiple L₁′ groups may each independently be the same or different.

In Formula 4-1 and Formula 4-2, Ar₁ may be a substituted or unsubstituted aryl group of 6 to 30 ring-forming carbon atoms, where a fluorenyl group is excluded. Here, in Formula 4-1, Ar₁ and Ar₁′ are not 1-naphthyl groups at the same time (e.g., simultaneously).

In Formula 4-3, R₄′ may be a hydrogen atom, a deuterium atom, a halogen atom, or a substituted or unsubstituted alkyl group of 1 to 20 carbon atoms, and/or combined with an adjacent group to form a ring, and R₅′ may be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring.

In Formula 4-3, n′ and p′ may each independently be an integer of 0 to 3. In some embodiments, when n′ is 2 or more, multiple L₂′ groups may each independently be the same or different, and when p′ is 2 or more, multiple R₄′ groups may each independently be the same or different.

In Formula 4-3, q′ may be an integer of 0 to 4. In some embodiments, when q′ is 2 or more, multiple R₅′ groups may each independently be the same or different.

In Formula 4-1 to Formula 4-3, X, Y, L₁, L₂, R₄, R₅, “m”, “n”, “p” and “q” may each independently be the same as defined in Formula 2-1 to Formula 2-3.

In an embodiment, Formula 1 may be represented by any one selected from among Formula 5-1 to Formula 5-3.

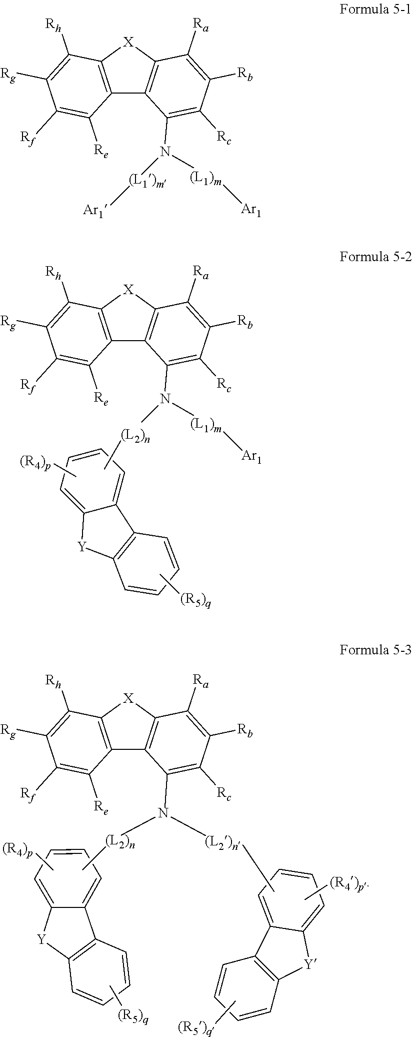

In Formula 5-1 to Formula 5-3, Rₑ may be a hydrogen atom or a deuterium atom, or combined with an adjacent R_{f} group to form a ring.

In Formula 5-1 to Formula 5-3, Rₐ to Rₑ may each independently be a hydrogen atom, a deuterium atom, or a halogen atom, or combined with an adjacent group to form a ring, and any one selected from among R_{f} to Rₕ may be a substituted or unsubstituted alkyl group of 1 to 20 carbon atoms or a substituted or unsubstituted aryl group of 6 to 30 ring-forming carbon atoms, and the remainder may each independently be a hydrogen atom, a deuterium atom, or a halogen atom, or combined with an adjacent group to form a ring, where a fluorenyl group is excluded.

In Formula 5-1, L₁′ may be a substituted or unsubstituted arylene group of 6 to 30 ring-forming carbon atoms.

In Formula 5-1, Ar₁′ may be a substituted or unsubstituted aryl group of 6 to 30 ring-forming carbon atoms, where a fluorenyl group is excluded.

In Formula 5-1, m′ may be an integer of 0 to 3. In some embodiments, when m′ is 2 or more, multiple L₁′ groups may each independently be the same or different.

In Formula 5-1 and Formula 5-2, Ar₁ may be a substituted or unsubstituted aryl group of 6 to 30 ring-forming carbon atoms, where a fluorenyl group is excluded. In Formula 5-1, Ar₁ and Ar₁′ are not 1-naphthyl groups at the same time (e.g., simultaneously).

In Formula 5-3, Y′ may be O or S, and L₂′ may be a substituted or unsubstituted arylene group of 6 to 30 ring-forming carbon atoms.

In Formula 5-3, R₄′ may be a hydrogen atom, a deuterium atom, a halogen atom, or a substituted or unsubstituted alkyl group of 1 to 20 carbon atoms, and/or combined with an adjacent group to form a ring.

In Formula 5-3, R₅′ may be a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring.

In Formula 5-3, n′ and p′ may each independently be an integer of 0 to 3. When n′ is 2 or more, multiple L₂′ groups may each independently be the same or different, and when p′ is 2 or more, multiple R₄′ groups may each independently be the same or different.

In Formula 5-3, q′ may be an integer of 0 to 4. In some embodiments, when q′ is 2 or more, multiple R₅′ groups may each independently be the same or different.

In Formula 5-1 to Formula 5-3, X, Y, L₁, L₂, R₄, R₅, “m”, “n”, “p” and “q” may each independently be the same as defined in Formula 2-1 to Formula 2-3.

In an embodiment, the amine compound represented by Formula 1 may not include an (e.g., additional) amine group other than the amine group represented by Formula 1. For example, the amine compound represented by Formula 1 may be a monoamine compound.

In an embodiment, the amine compound represented by Formula 1 may not include an N-containing heteroaryl group.

In an embodiment, L₁ and L₂ of Formula 2-2 and Formula 2-3 may be each independently a direct linkage, a substituted or unsubstituted phenylene group, a substituted or unsubstituted divalent biphenyl group or a substituted or unsubstituted naphthylenyl group.

In an embodiment, Rₕ may be a substituted or unsubstituted phenyl group.

The amine compound represented by Formula 1 according to an embodiment may be any one selected from among the compounds represented in Compound Group 1 to Compound Group 3. However, embodiments of the present disclosure are not limited thereto.

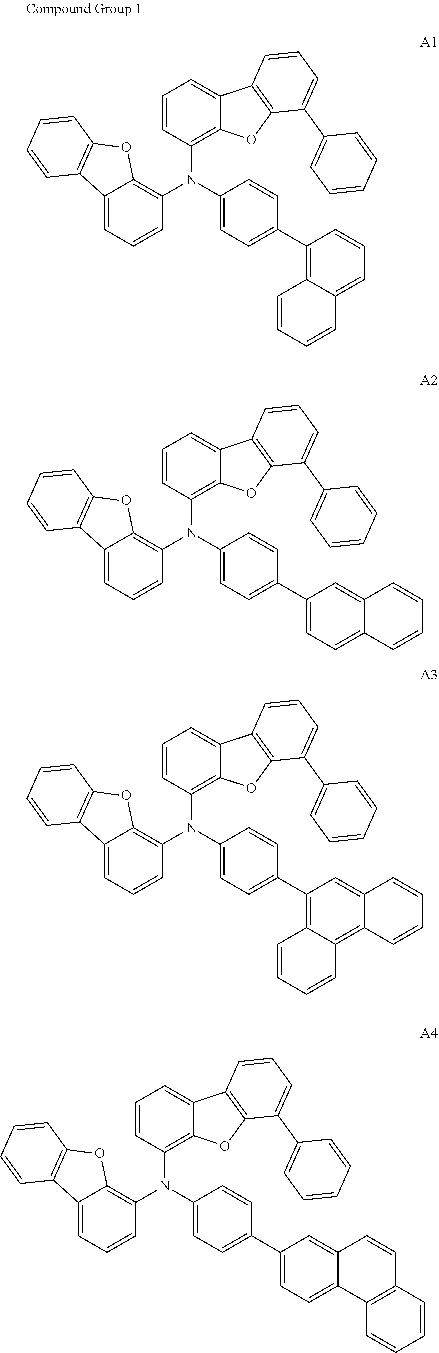 [SMILES: c1ccc(-c2cccc3c2oc2c(N(c4ccc(-c5cccc6ccccc56)cc4)c4cccc5c4oc4ccccc45)cccc23)cc1 ; c1ccc(-c2cccc3c2oc2c(N(c4ccc(-c5ccc6ccccc6c5)cc4)c4cccc5c4oc4ccccc45)cccc23)cc1 ; c1ccc(-c2cccc3c2oc2c(N(c4ccc(-c5cc6ccccc6c6ccccc56)cc4)c4cccc5c4oc4ccccc45)cccc23)cc1 ; c1ccc(-c2cccc3c2oc2c(N(c4ccc(-c5ccc6c(ccc7ccccc76)c5)cc4)c4cccc5c4oc4ccccc45)cccc23)cc1]

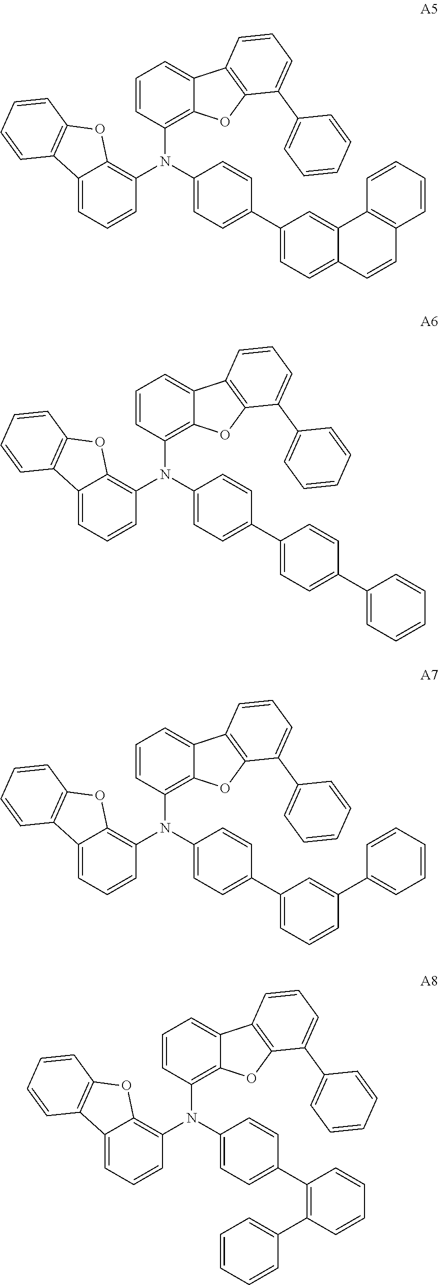 [SMILES: c1ccc(-c2cccc3c2oc2c(N(c4ccc(-c5ccc6ccc7ccccc7c6c5)cc4)c4cccc5c4oc4ccccc45)cccc23)cc1 ; c1ccc(-c2ccc(-c3ccc(N(c4cccc5c4oc4ccccc45)c4cccc5c4oc4c(-c6ccccc6)cccc45)cc3)cc2)cc1 ; c1ccc(-c2cccc(-c3ccc(N(c4cccc5c4oc4ccccc45)c4cccc5c4oc4c(-c6ccccc6)cccc45)cc3)c2)cc1 ; c1ccc(-c2ccccc2-c2ccc(N(c3cccc4c3oc3ccccc34)c3cccc4c3oc3c(-c5ccccc5)cccc34)cc2)cc1]

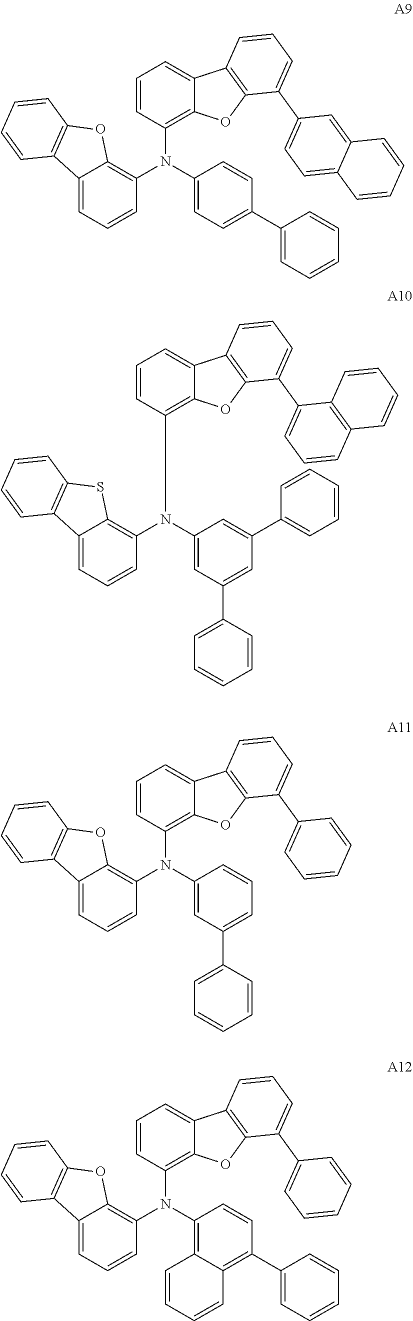 [SMILES: c1ccc(-c2ccc(N(c3cccc4c3oc3ccccc34)c3cccc4c3oc3c(-c5ccc6ccccc6c5)cccc34)cc2)cc1 ; c1ccc(-c2cc(-c3ccccc3)cc(N(c3cccc4c3oc3c(-c5cccc6ccccc56)cccc34)c3cccc4c3sc3ccccc34)c2)cc1 ; c1ccc(-c2cccc(N(c3cccc4c3oc3ccccc34)c3cccc4c3oc3c(-c5ccccc5)cccc34)c2)cc1 ; c1ccc(-c2ccc(N(c3cccc4c3oc3ccccc34)c3cccc4c3oc3c(-c5ccccc5)cccc34)c3ccccc23)cc1]

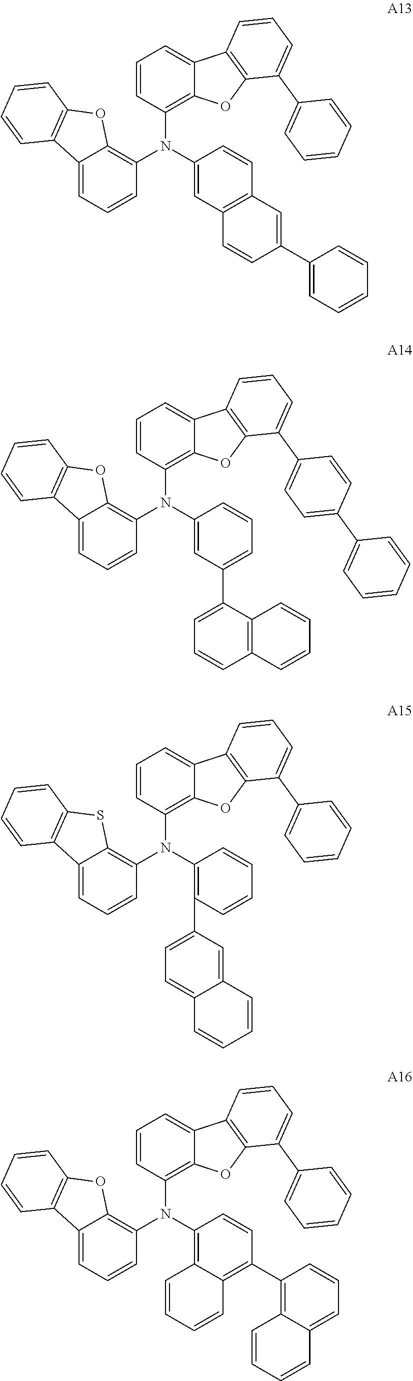

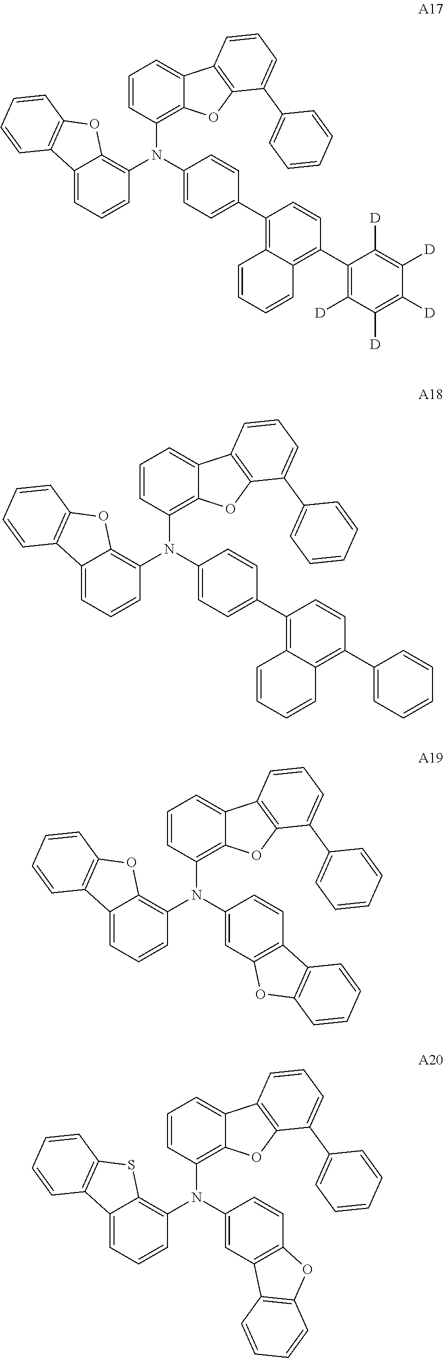

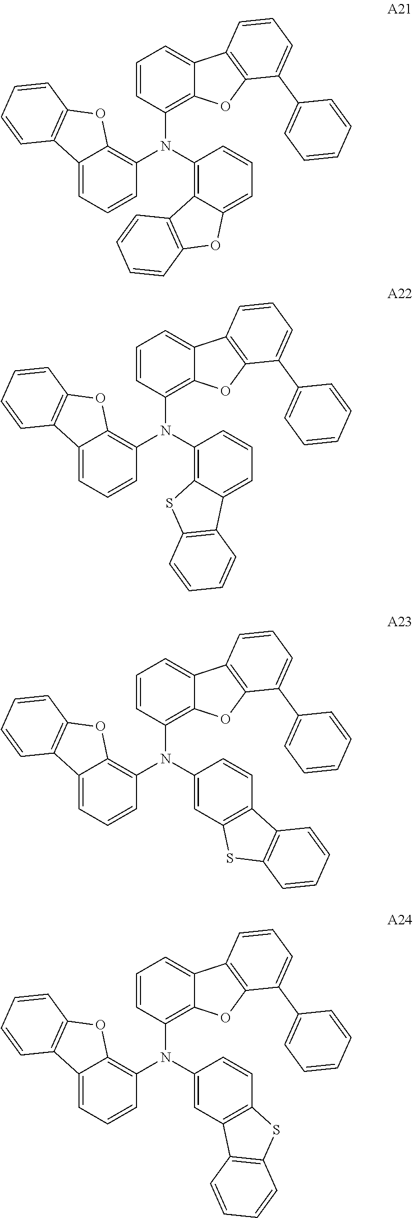

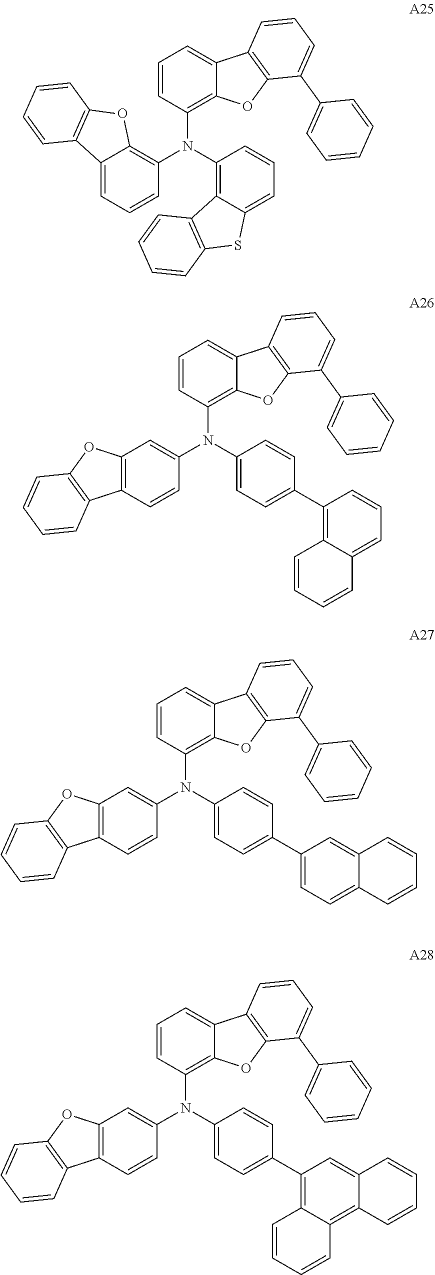 [SMILES: c1ccc(-c2cccc3c2oc2c(N(c4cccc5c4oc4ccccc45)c4cccc5sc6ccccc6c45)cccc23)cc1 ; c1ccc(-c2cccc3c2oc2c(N(c4ccc(-c5cccc6ccccc56)cc4)c4ccc5c(c4)oc4ccccc45)cccc23)cc1 ; c1ccc(-c2cccc3c2oc2c(N(c4ccc(-c5ccc6ccccc6c5)cc4)c4ccc5c(c4)oc4ccccc45)cccc23)cc1 ; c1ccc(-c2cccc3c2oc2c(N(c4ccc(-c5cc6ccccc6c6ccccc56)cc4)c4ccc5c(c4)oc4ccccc45)cccc23)cc1]

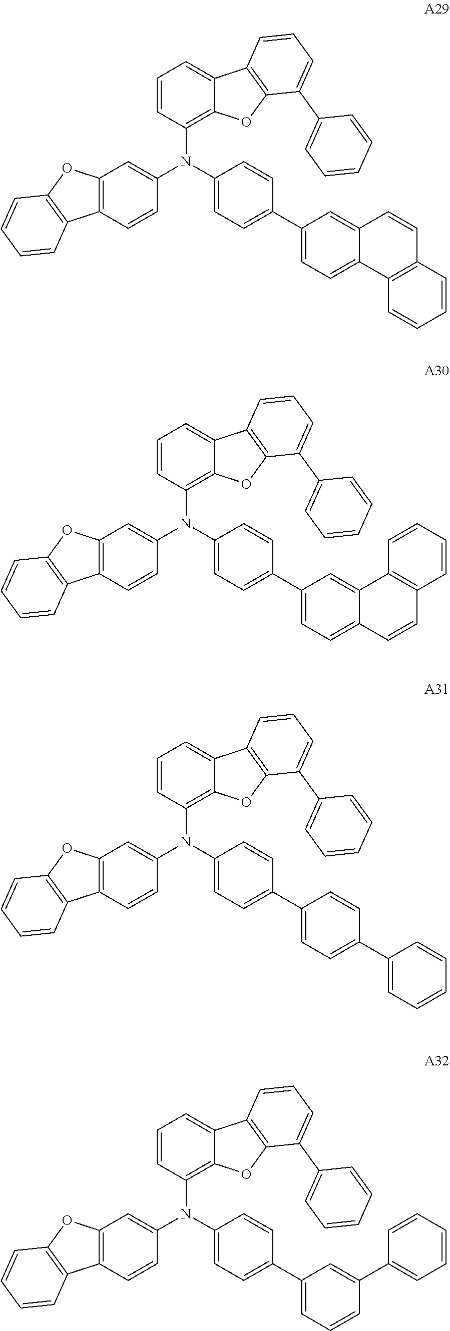 [SMILES: c1ccc(-c2cccc3c2oc2c(N(c4ccc(-c5ccc6c(ccc7ccccc76)c5)cc4)c4ccc5c(c4)oc4ccccc45)cccc23)cc1 ; c1ccc(-c2cccc3c2oc2c(N(c4ccc(-c5ccc6ccc7ccccc7c6c5)cc4)c4ccc5c(c4)oc4ccccc45)cccc23)cc1 ; c1ccc(-c2ccc(-c3ccc(N(c4ccc5c(c4)oc4ccccc45)c4cccc5c4oc4c(-c6ccccc6)cccc45)cc3)cc2)cc1 ; c1ccc(-c2cccc(-c3ccc(N(c4ccc5c(c4)oc4ccccc45)c4cccc5c4oc4c(-c6ccccc6)cccc45)cc3)c2)cc1]

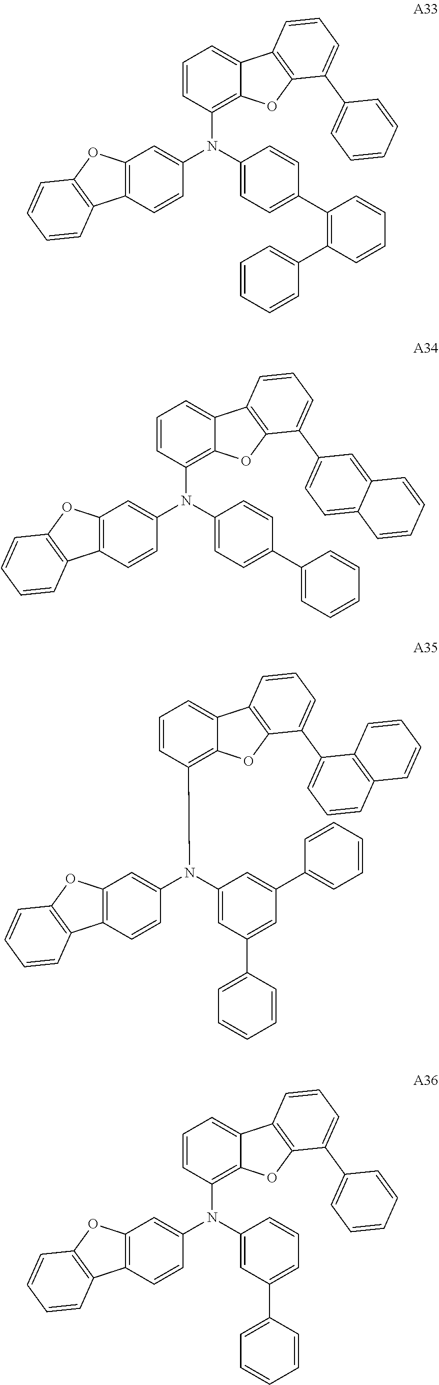 [SMILES: c1ccc(-c2ccccc2-c2ccc(N(c3ccc4c(c3)oc3ccccc34)c3cccc4c3oc3c(-c5ccccc5)cccc34)cc2)cc1 ; c1ccc(-c2ccc(N(c3ccc4c(c3)oc3ccccc34)c3cccc4c3oc3c(-c5ccc6ccccc6c5)cccc34)cc2)cc1 ; c1ccc(-c2cc(-c3ccccc3)cc(N(c3ccc4c(c3)oc3ccccc34)c3cccc4c3oc3c(-c5cccc6ccccc56)cccc34)c2)cc1 ; c1ccc(-c2cccc(N(c3ccc4c(c3)oc3ccccc34)c3cccc4c3oc3c(-c5ccccc5)cccc34)c2)cc1]

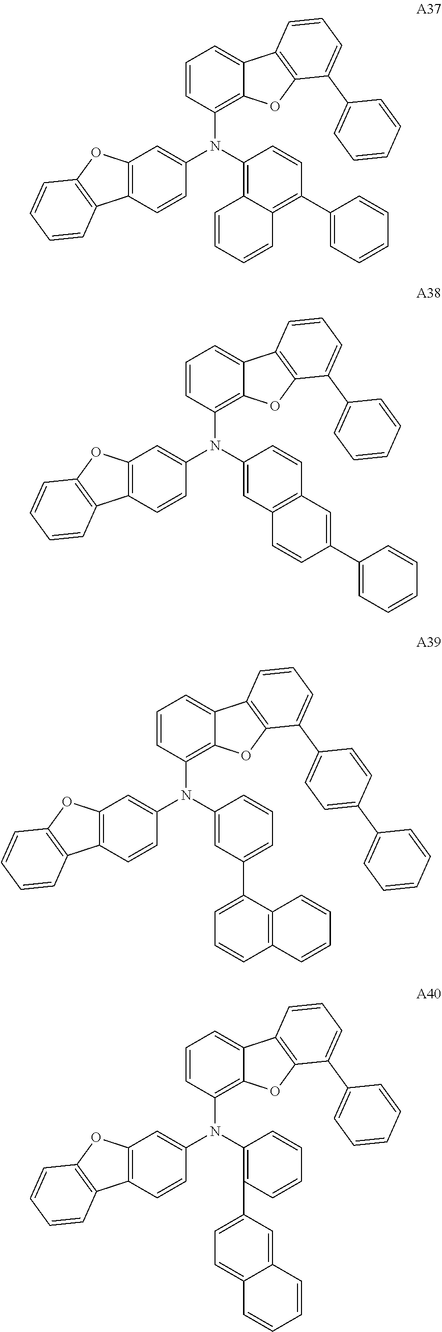

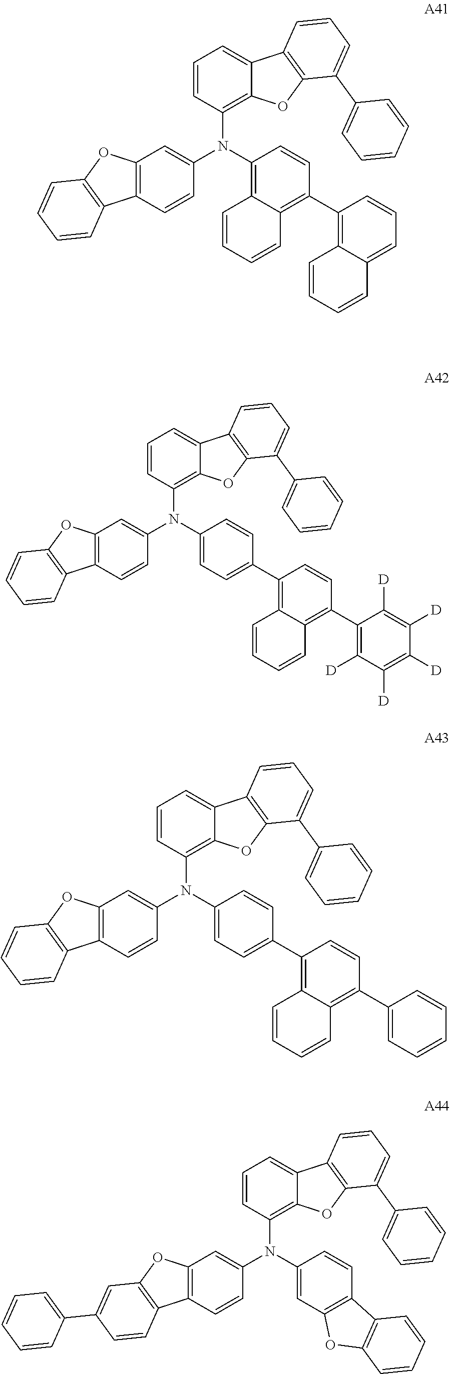 [SMILES: c1ccc(-c2cccc3c2oc2c(N(c4ccc5c(c4)oc4ccccc45)c4ccc(-c5cccc6ccccc56)c5ccccc45)cccc23)cc1 ; [2H]c1c([2H])c([2H])c(-c2ccc(-c3ccc(N(c4ccc5c(c4)oc4ccccc45)c4cccc5c4oc4c(-c6ccccc6)cccc45)cc3)c3ccccc23)c([2H])c1[2H] ; c1ccc(-c2ccc(-c3ccc(N(c4ccc5c(c4)oc4ccccc45)c4cccc5c4oc4c(-c6ccccc6)cccc45)cc3)c3ccccc23)cc1 ; c1ccc(-c2ccc3c(c2)oc2cc(N(c4ccc5c(c4)oc4ccccc45)c4cccc5c4oc4c(-c6ccccc6)cccc45)ccc23)cc1]

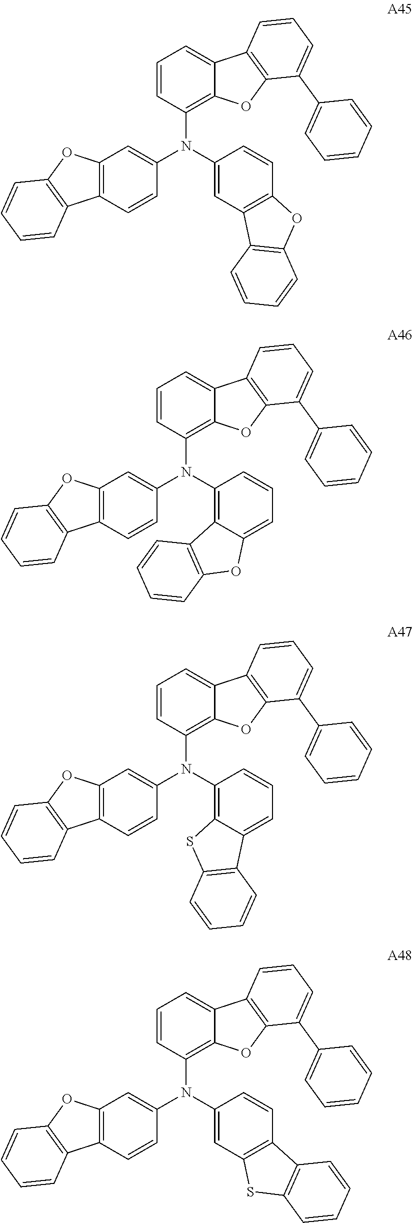

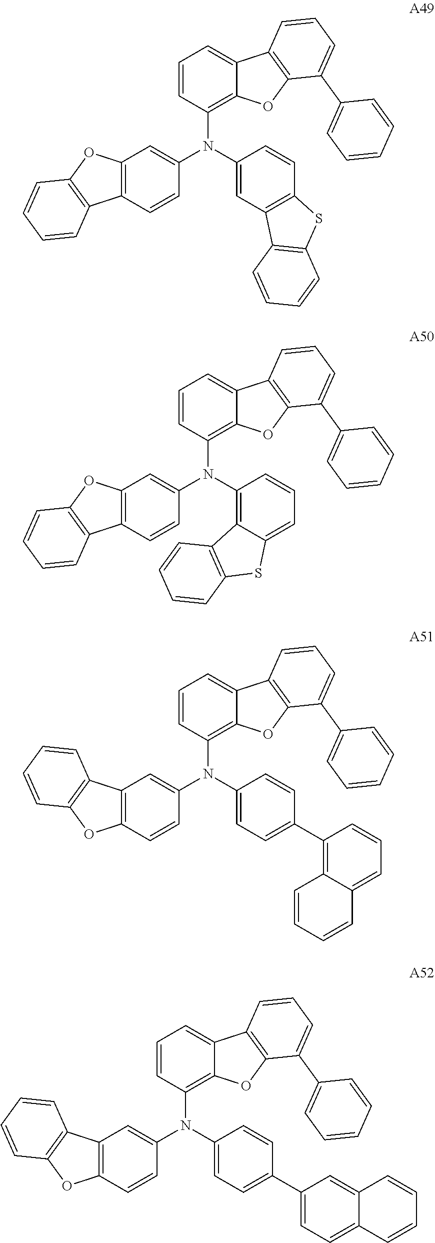

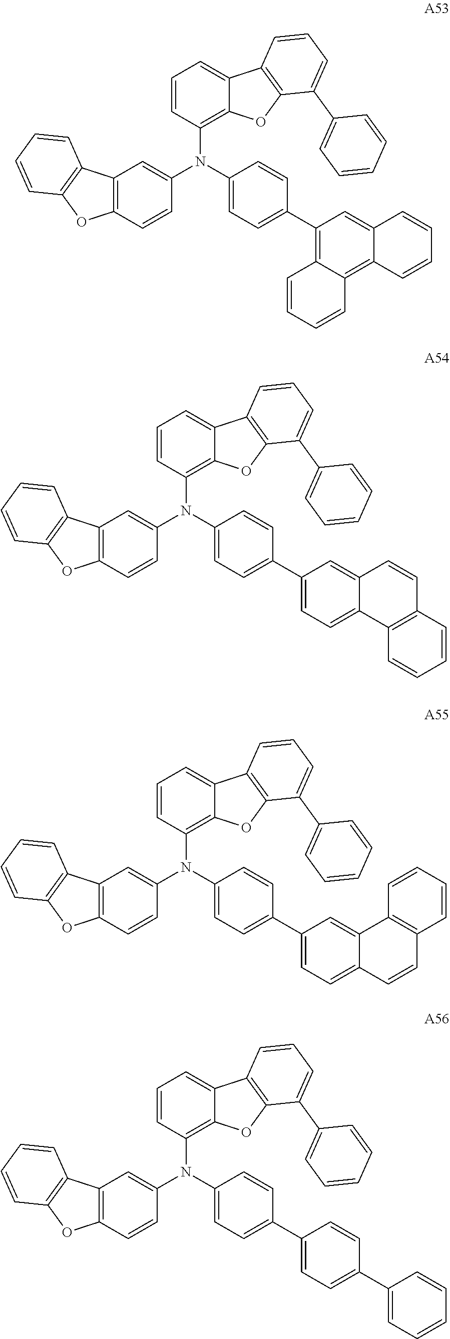 [SMILES: c1ccc(-c2cccc3c2oc2c(N(c4ccc(-c5cc6ccccc6c6ccccc56)cc4)c4ccc5oc6ccccc6c5c4)cccc23)cc1 ; c1ccc(-c2cccc3c2oc2c(N(c4ccc(-c5ccc6c(ccc7ccccc76)c5)cc4)c4ccc5oc6ccccc6c5c4)cccc23)cc1 ; c1ccc(-c2cccc3c2oc2c(N(c4ccc(-c5ccc6ccc7ccccc7c6c5)cc4)c4ccc5oc6ccccc6c5c4)cccc23)cc1 ; c1ccc(-c2ccc(-c3ccc(N(c4ccc5oc6ccccc6c5c4)c4cccc5c4oc4c(-c6ccccc6)cccc45)cc3)cc2)cc1]

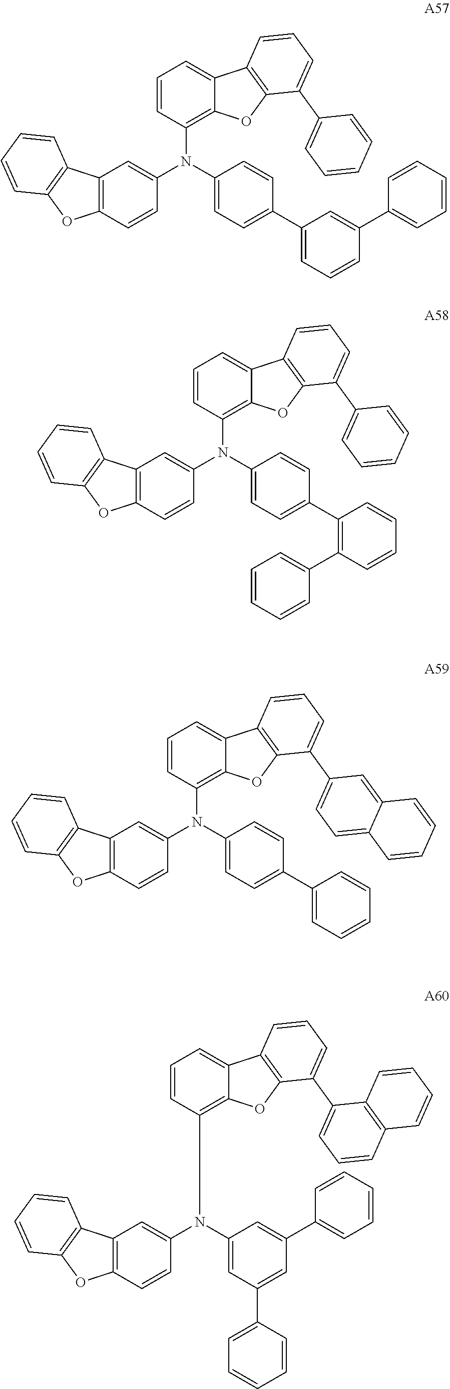 [SMILES: c1ccc(-c2cccc(-c3ccc(N(c4ccc5oc6ccccc6c5c4)c4cccc5c4oc4c(-c6ccccc6)cccc45)cc3)c2)cc1 ; c1ccc(-c2ccccc2-c2ccc(N(c3ccc4oc5ccccc5c4c3)c3cccc4c3oc3c(-c5ccccc5)cccc34)cc2)cc1 ; c1ccc(-c2ccc(N(c3ccc4oc5ccccc5c4c3)c3cccc4c3oc3c(-c5ccc6ccccc6c5)cccc34)cc2)cc1 ; c1ccc(-c2cc(-c3ccccc3)cc(N(c3ccc4oc5ccccc5c4c3)c3cccc4c3oc3c(-c5cccc6ccccc56)cccc34)c2)cc1]

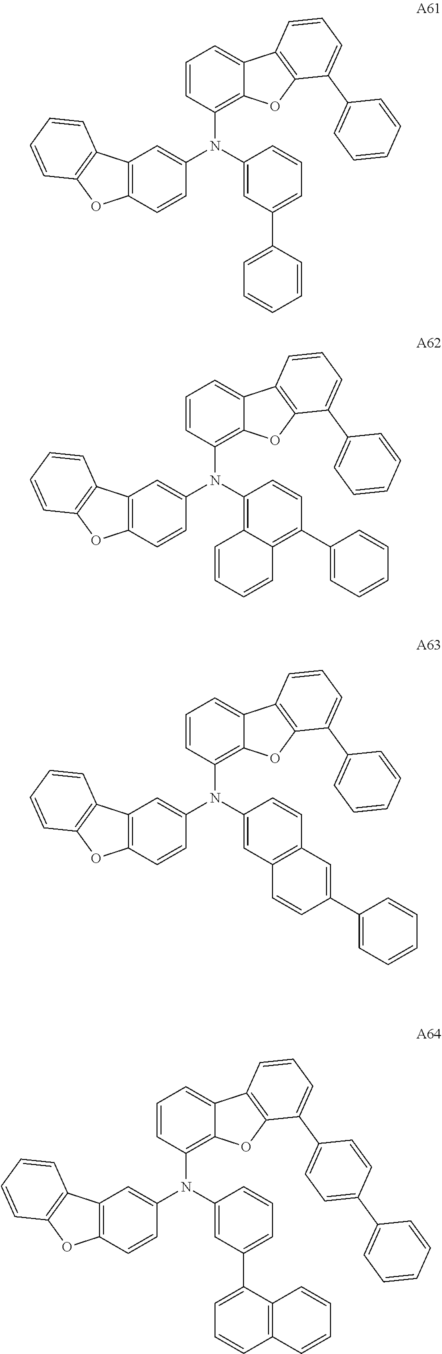

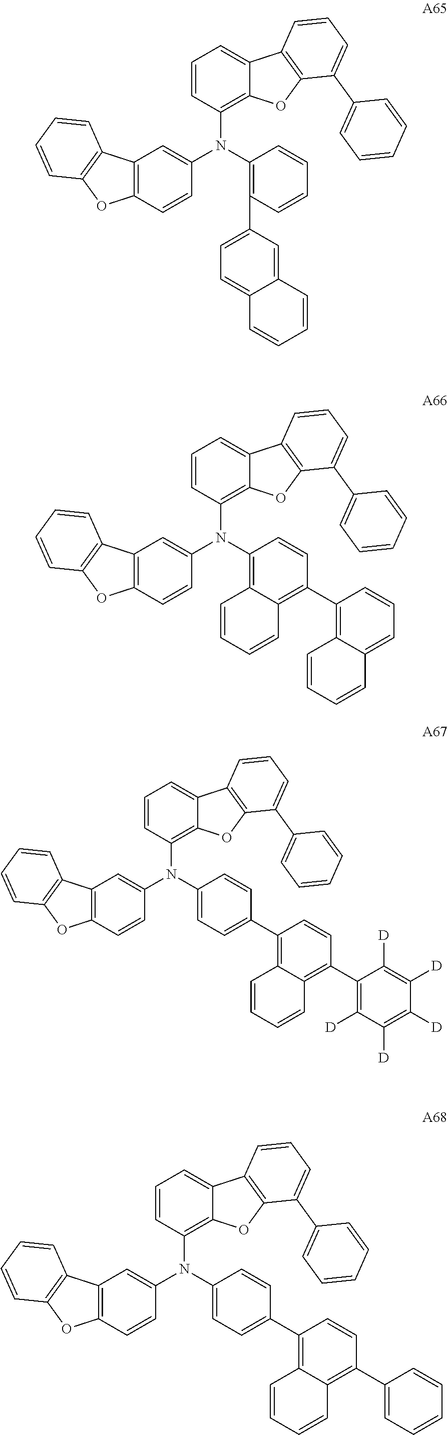 [SMILES: c1ccc(-c2cccc3c2oc2c(N(c4ccc5oc6ccccc6c5c4)c4ccccc4-c4ccc5ccccc5c4)cccc23)cc1 ; c1ccc(-c2cccc3c2oc2c(N(c4ccc5oc6ccccc6c5c4)c4ccc(-c5cccc6ccccc56)c5ccccc45)cccc23)cc1 ; [2H]c1c([2H])c([2H])c(-c2ccc(-c3ccc(N(c4ccc5oc6ccccc6c5c4)c4cccc5c4oc4c(-c6ccccc6)cccc45)cc3)c3ccccc23)c([2H])c1[2H] ; c1ccc(-c2ccc(-c3ccc(N(c4ccc5oc6ccccc6c5c4)c4cccc5c4oc4c(-c6ccccc6)cccc45)cc3)c3ccccc23)cc1]

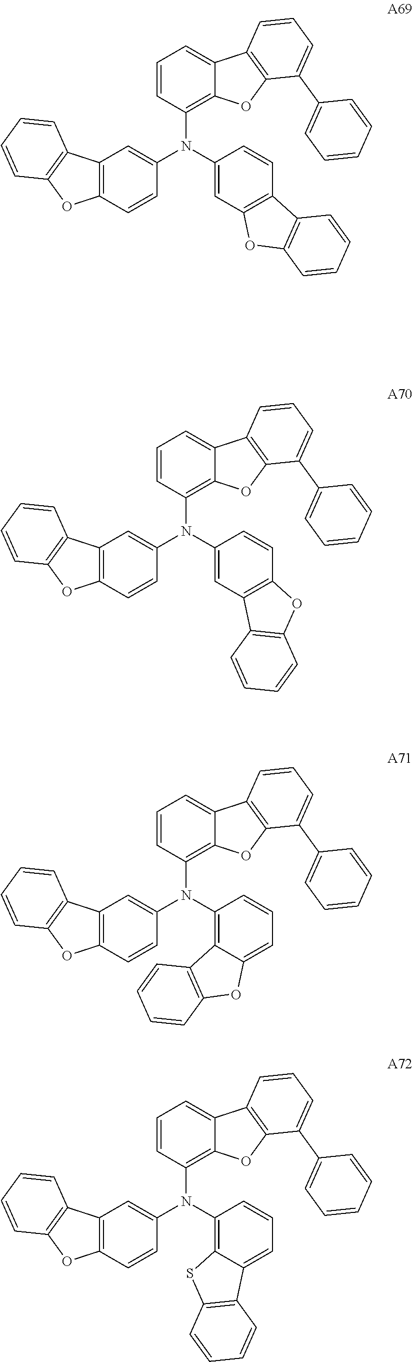 [SMILES: c1ccc(-c2cccc3c2oc2c(N(c4ccc5c(c4)oc4ccccc45)c4ccc5oc6ccccc6c5c4)cccc23)cc1 ; c1ccc(-c2cccc3c2oc2c(N(c4ccc5oc6ccccc6c5c4)c4ccc5oc6ccccc6c5c4)cccc23)cc1 ; c1ccc(-c2cccc3c2oc2c(N(c4ccc5oc6ccccc6c5c4)c4cccc5oc6ccccc6c45)cccc23)cc1 ; c1ccc(-c2cccc3c2oc2c(N(c4ccc5oc6ccccc6c5c4)c4cccc5c4sc4ccccc45)cccc23)cc1]

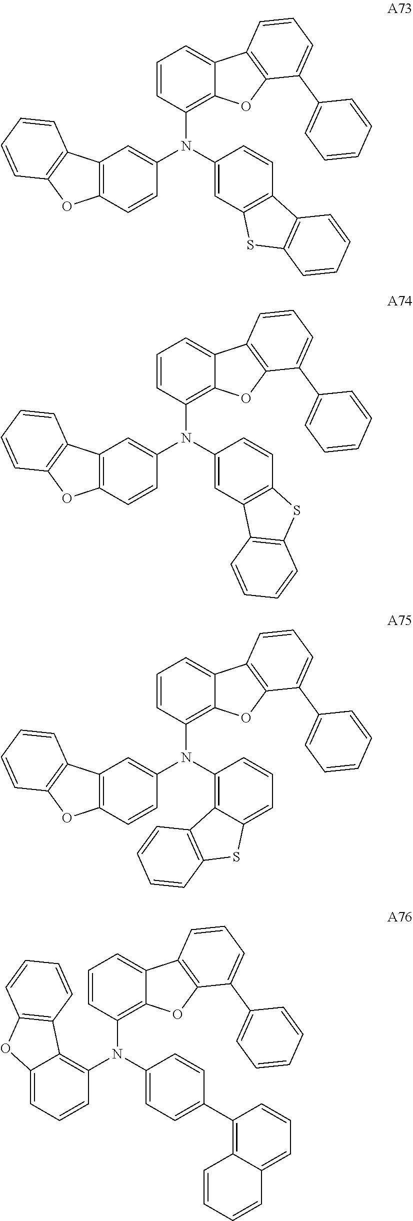

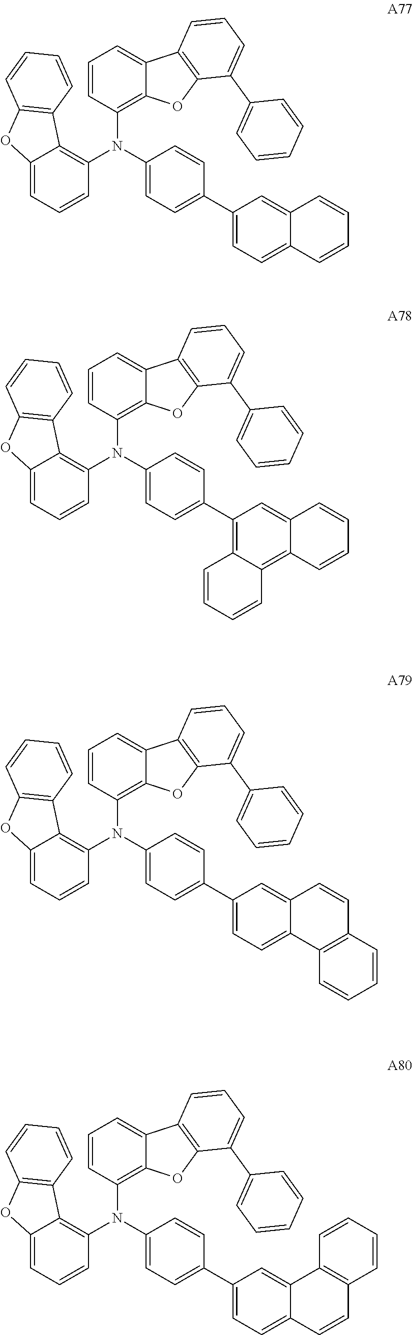 [SMILES: c1ccc(-c2cccc3c2oc2c(N(c4ccc(-c5ccc6ccccc6c5)cc4)c4cccc5oc6ccccc6c45)cccc23)cc1 ; c1ccc(-c2cccc3c2oc2c(N(c4ccc(-c5cc6ccccc6c6ccccc56)cc4)c4cccc5oc6ccccc6c45)cccc23)cc1 ; c1ccc(-c2cccc3c2oc2c(N(c4ccc(-c5ccc6c(ccc7ccccc76)c5)cc4)c4cccc5oc6ccccc6c45)cccc23)cc1 ; c1ccc(-c2cccc3c2oc2c(N(c4ccc(-c5ccc6ccc7ccccc7c6c5)cc4)c4cccc5oc6ccccc6c45)cccc23)cc1]

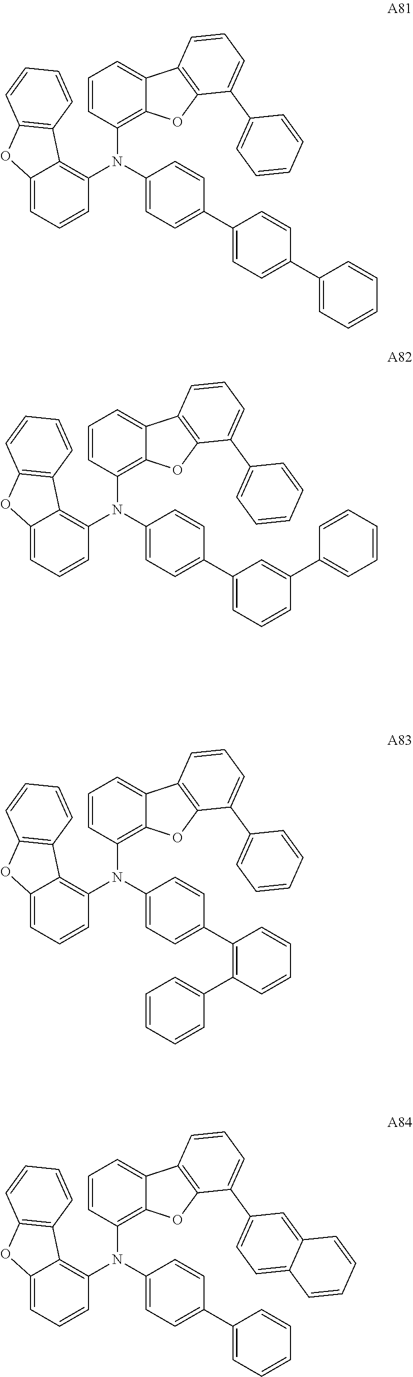 [SMILES: c1ccc(-c2ccc(-c3ccc(N(c4cccc5c4oc4c(-c6ccccc6)cccc45)c4cccc5oc6ccccc6c45)cc3)cc2)cc1 ; c1ccc(-c2cccc(-c3ccc(N(c4cccc5c4oc4c(-c6ccccc6)cccc45)c4cccc5oc6ccccc6c45)cc3)c2)cc1 ; c1ccc(-c2ccccc2-c2ccc(N(c3cccc4c3oc3c(-c5ccccc5)cccc34)c3cccc4oc5ccccc5c34)cc2)cc1 ; c1ccc(-c2ccc(N(c3cccc4c3oc3c(-c5ccc6ccccc6c5)cccc34)c3cccc4oc5ccccc5c34)cc2)cc1]

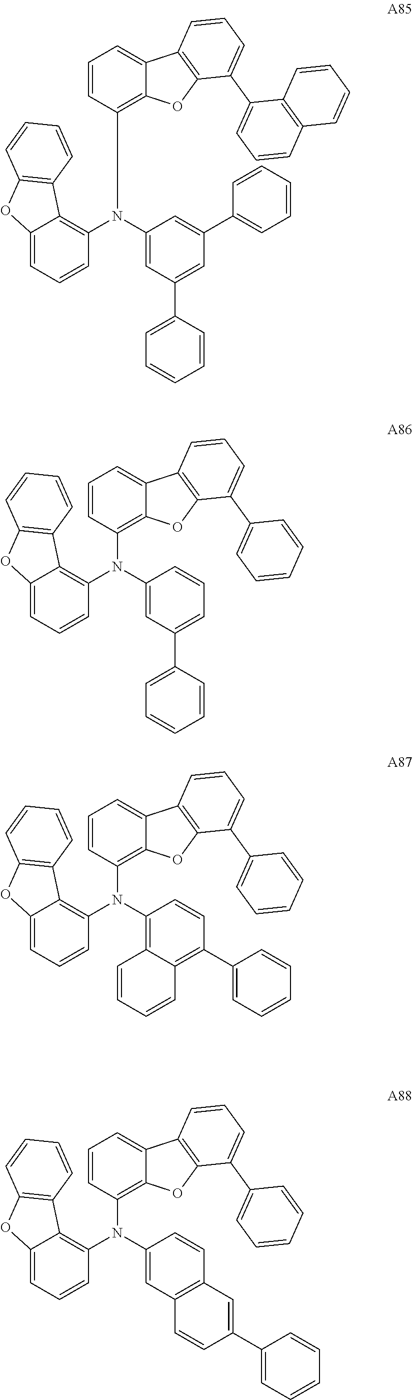

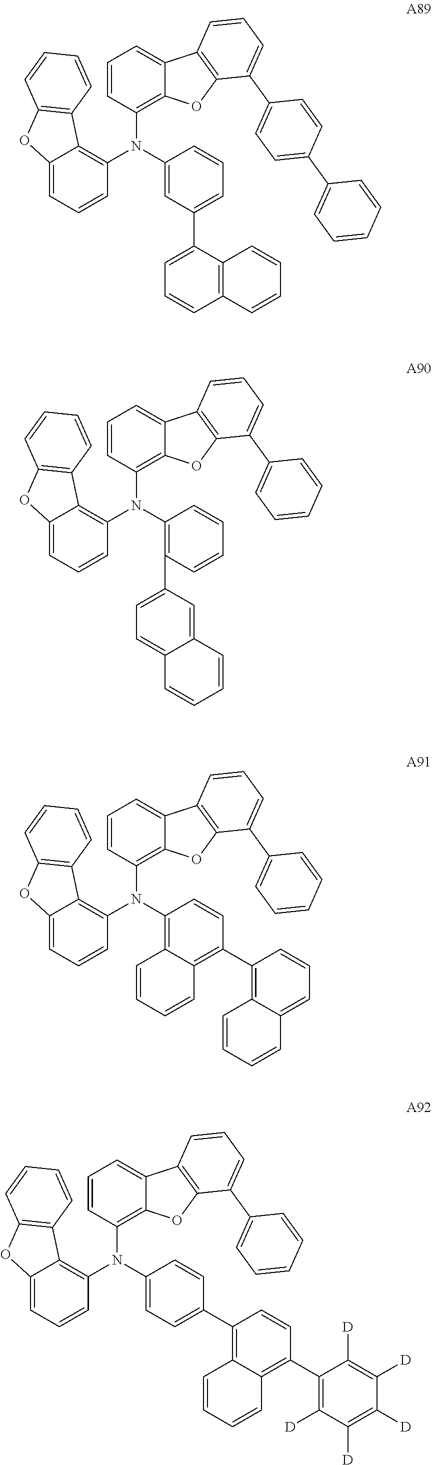 [SMILES: c1ccc(-c2ccc(-c3cccc4c3oc3c(N(c5cccc(-c6cccc7ccccc67)c5)c5cccc6oc7ccccc7c56)cccc34)cc2)cc1 ; c1ccc(-c2cccc3c2oc2c(N(c4ccccc4-c4ccc5ccccc5c4)c4cccc5oc6ccccc6c45)cccc23)cc1 ; c1ccc(-c2cccc3c2oc2c(N(c4ccc(-c5cccc6ccccc56)c5ccccc45)c4cccc5oc6ccccc6c45)cccc23)cc1 ; [2H]c1c([2H])c([2H])c(-c2ccc(-c3ccc(N(c4cccc5c4oc4c(-c6ccccc6)cccc45)c4cccc5oc6ccccc6c45)cc3)c3ccccc23)c([2H])c1[2H]]

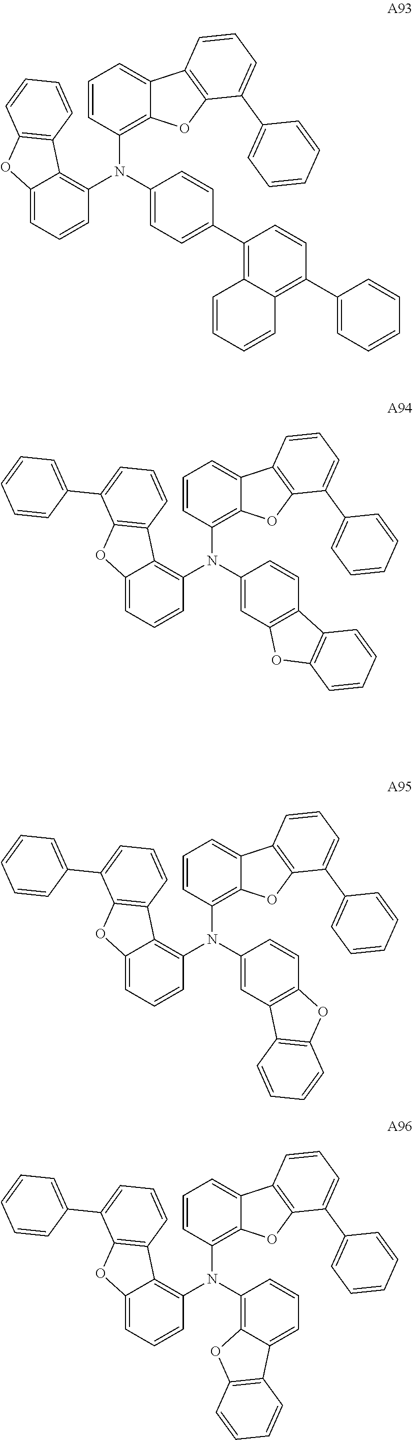 [SMILES: c1ccc(-c2ccc(-c3ccc(N(c4cccc5c4oc4c(-c6ccccc6)cccc45)c4cccc5oc6ccccc6c45)cc3)c3ccccc23)cc1 ; c1ccc(-c2cccc3c2oc2c(N(c4ccc5c(c4)oc4ccccc45)c4cccc5oc6c(-c7ccccc7)cccc6c45)cccc23)cc1 ; c1ccc(-c2cccc3c2oc2c(N(c4ccc5oc6ccccc6c5c4)c4cccc5oc6c(-c7ccccc7)cccc6c45)cccc23)cc1 ; c1ccc(-c2cccc3c2oc2c(N(c4cccc5c4oc4ccccc45)c4cccc5oc6c(-c7ccccc7)cccc6c45)cccc23)cc1]

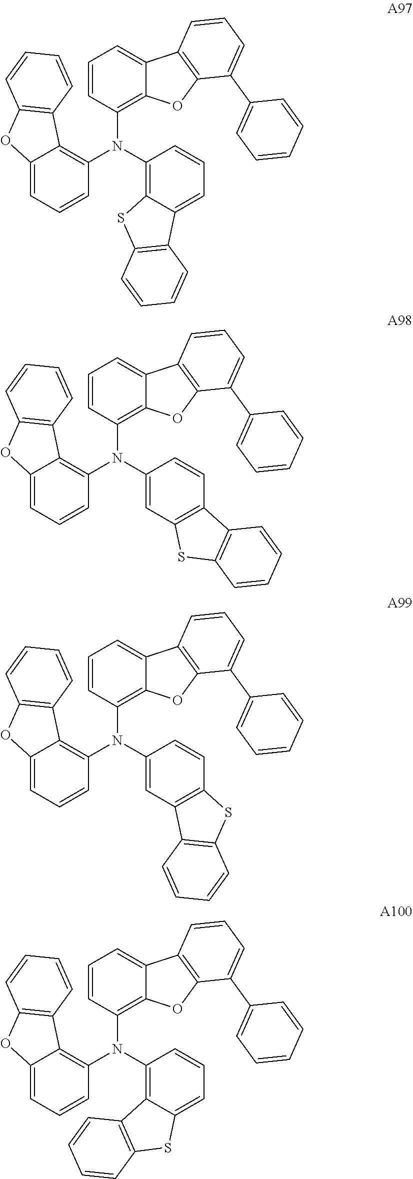

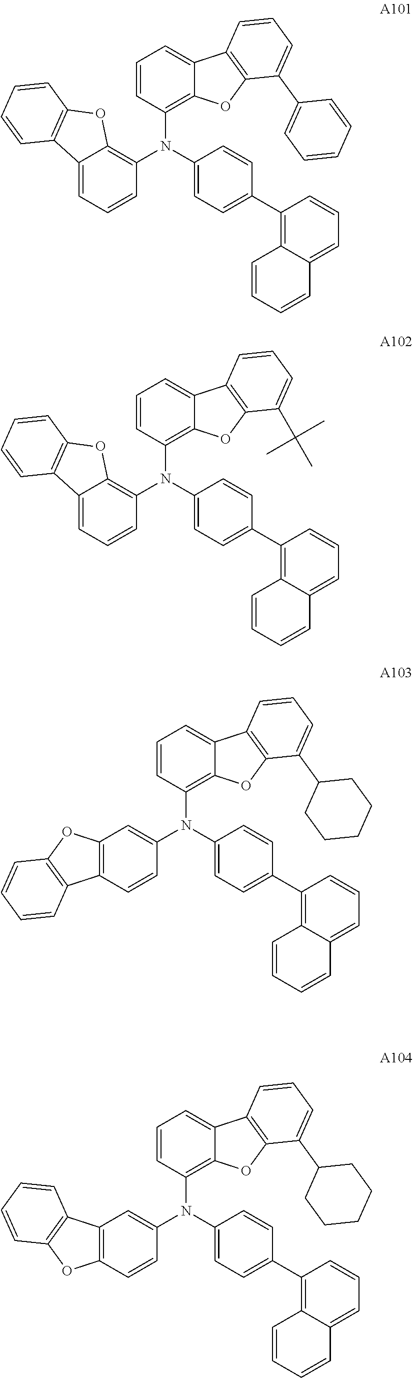

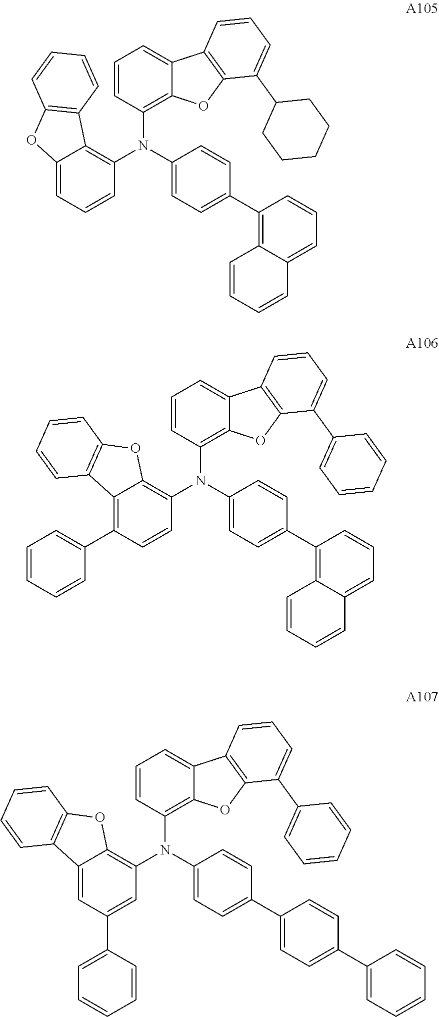

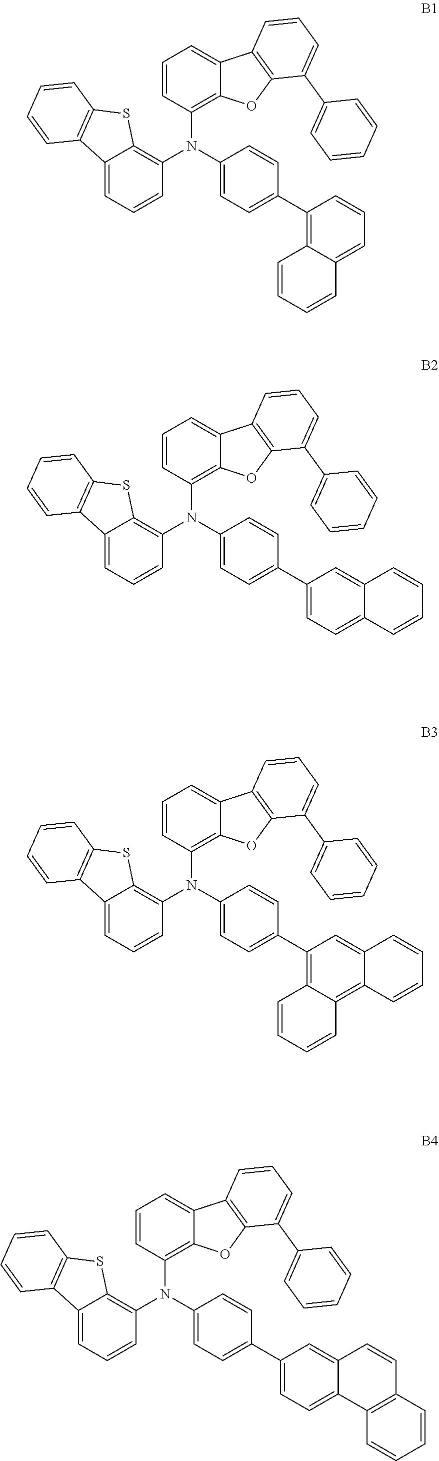 [SMILES: c1ccc(-c2cccc3c2oc2c(N(c4ccc(-c5cccc6ccccc56)cc4)c4cccc5c4sc4ccccc45)cccc23)cc1 ; c1ccc(-c2cccc3c2oc2c(N(c4ccc(-c5ccc6ccccc6c5)cc4)c4cccc5c4sc4ccccc45)cccc23)cc1 ; c1ccc(-c2cccc3c2oc2c(N(c4ccc(-c5cc6ccccc6c6ccccc56)cc4)c4cccc5c4sc4ccccc45)cccc23)cc1 ; c1ccc(-c2cccc3c2oc2c(N(c4ccc(-c5ccc6c(ccc7ccccc76)c5)cc4)c4cccc5c4sc4ccccc45)cccc23)cc1]

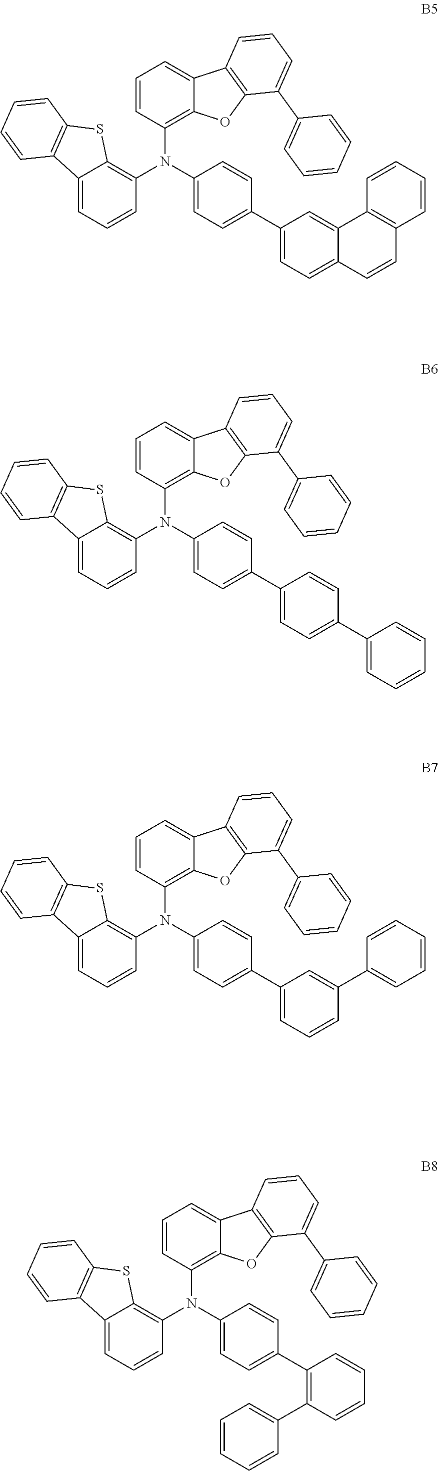 [SMILES: c1ccc(-c2cccc3c2oc2c(N(c4ccc(-c5ccc6ccc7ccccc7c6c5)cc4)c4cccc5c4sc4ccccc45)cccc23)cc1 ; c1ccc(-c2ccc(-c3ccc(N(c4cccc5c4oc4c(-c6ccccc6)cccc45)c4cccc5c4sc4ccccc45)cc3)cc2)cc1 ; c1ccc(-c2cccc(-c3ccc(N(c4cccc5c4oc4c(-c6ccccc6)cccc45)c4cccc5c4sc4ccccc45)cc3)c2)cc1 ; c1ccc(-c2ccccc2-c2ccc(N(c3cccc4c3oc3c(-c5ccccc5)cccc34)c3cccc4c3sc3ccccc34)cc2)cc1]

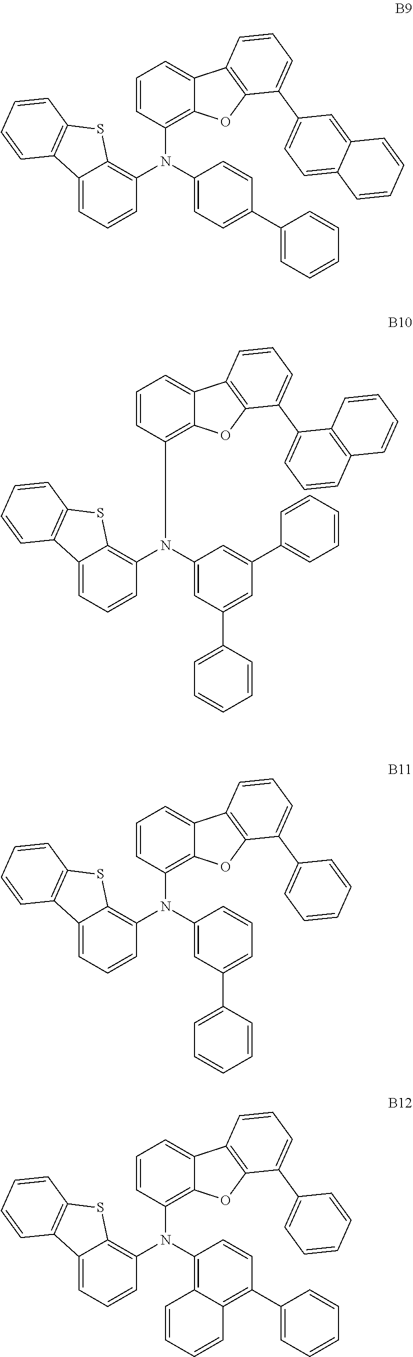

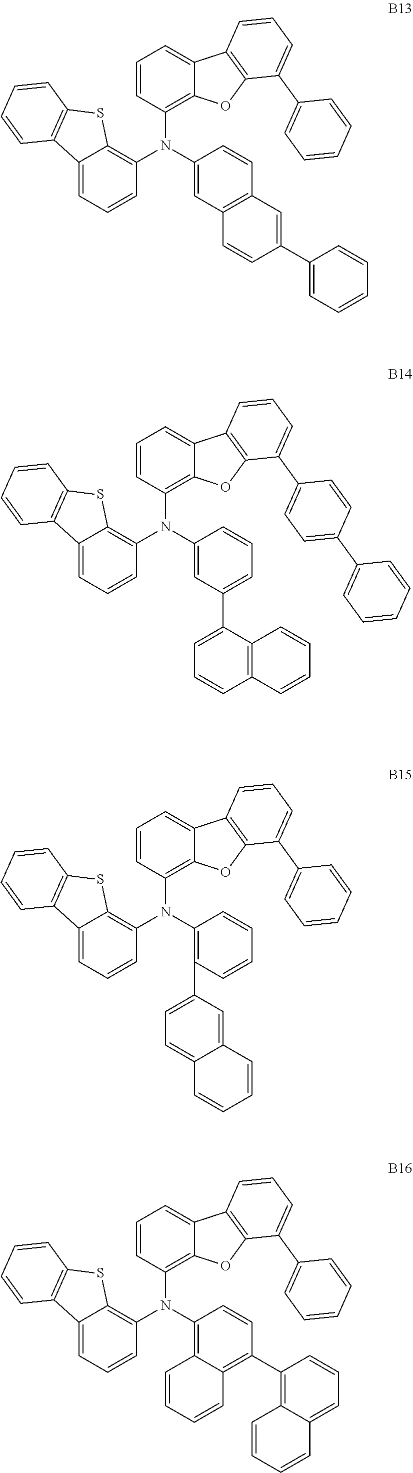 [SMILES: c1ccc(-c2ccc3cc(N(c4cccc5c4oc4c(-c6ccccc6)cccc45)c4cccc5c4sc4ccccc45)ccc3c2)cc1 ; c1ccc(-c2ccc(-c3cccc4c3oc3c(N(c5cccc(-c6cccc7ccccc67)c5)c5cccc6c5sc5ccccc56)cccc34)cc2)cc1 ; c1ccc(-c2cccc3c2oc2c(N(c4ccccc4-c4ccc5ccccc5c4)c4cccc5c4sc4ccccc45)cccc23)cc1 ; c1ccc(-c2cccc3c2oc2c(N(c4ccc(-c5cccc6ccccc56)c5ccccc45)c4cccc5c4sc4ccccc45)cccc23)cc1]

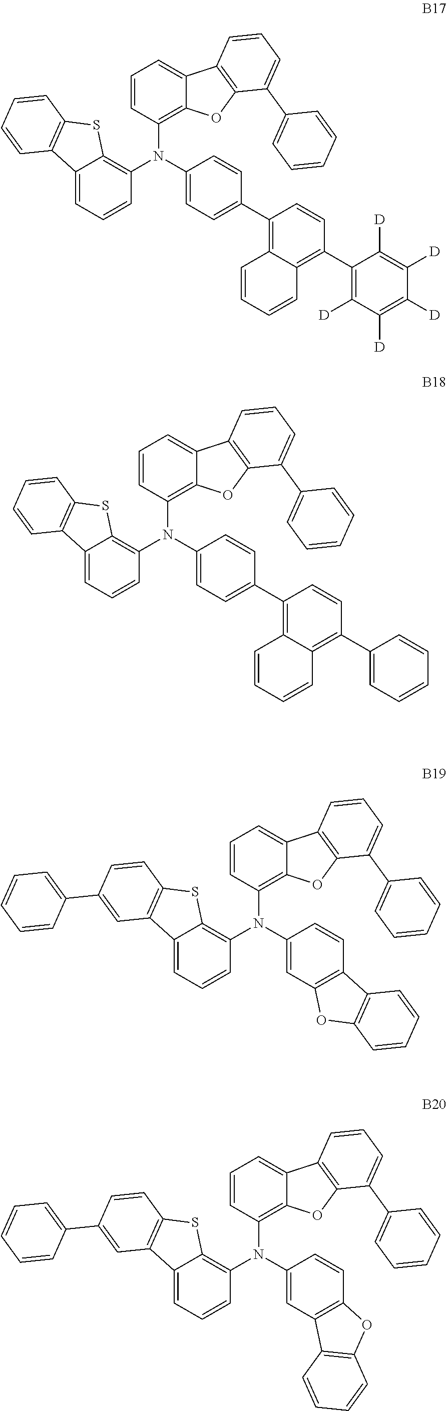 [SMILES: [2H]c1c([2H])c([2H])c(-c2ccc(-c3ccc(N(c4cccc5c4oc4c(-c6ccccc6)cccc45)c4cccc5c4sc4ccccc45)cc3)c3ccccc23)c([2H])c1[2H] ; c1ccc(-c2ccc(-c3ccc(N(c4cccc5c4oc4c(-c6ccccc6)cccc45)c4cccc5c4sc4ccccc45)cc3)c3ccccc23)cc1 ; c1ccc(-c2ccc3sc4c(N(c5ccc6c(c5)oc5ccccc56)c5cccc6c5oc5c(-c7ccccc7)cccc56)cccc4c3c2)cc1 ; c1ccc(-c2ccc3sc4c(N(c5ccc6oc7ccccc7c6c5)c5cccc6c5oc5c(-c7ccccc7)cccc56)cccc4c3c2)cc1]

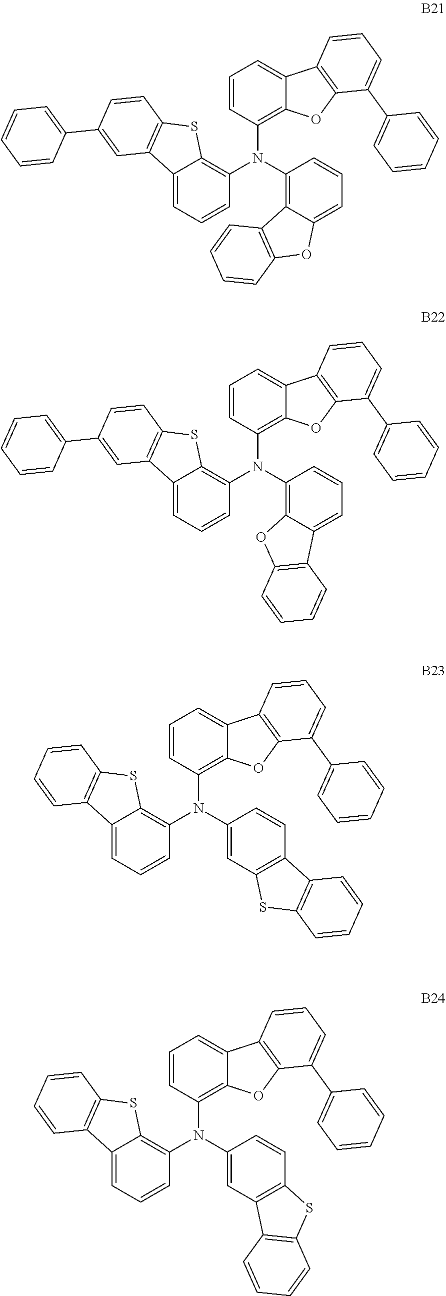 [SMILES: c1ccc(-c2ccc3sc4c(N(c5cccc6c5oc5c(-c7ccccc7)cccc56)c5cccc6oc7ccccc7c56)cccc4c3c2)cc1 ; c1ccc(-c2ccc3sc4c(N(c5cccc6c5oc5ccccc56)c5cccc6c5oc5c(-c7ccccc7)cccc56)cccc4c3c2)cc1 ; c1ccc(-c2cccc3c2oc2c(N(c4ccc5c(c4)sc4ccccc45)c4cccc5c4sc4ccccc45)cccc23)cc1 ; c1ccc(-c2cccc3c2oc2c(N(c4ccc5sc6ccccc6c5c4)c4cccc5c4sc4ccccc45)cccc23)cc1]

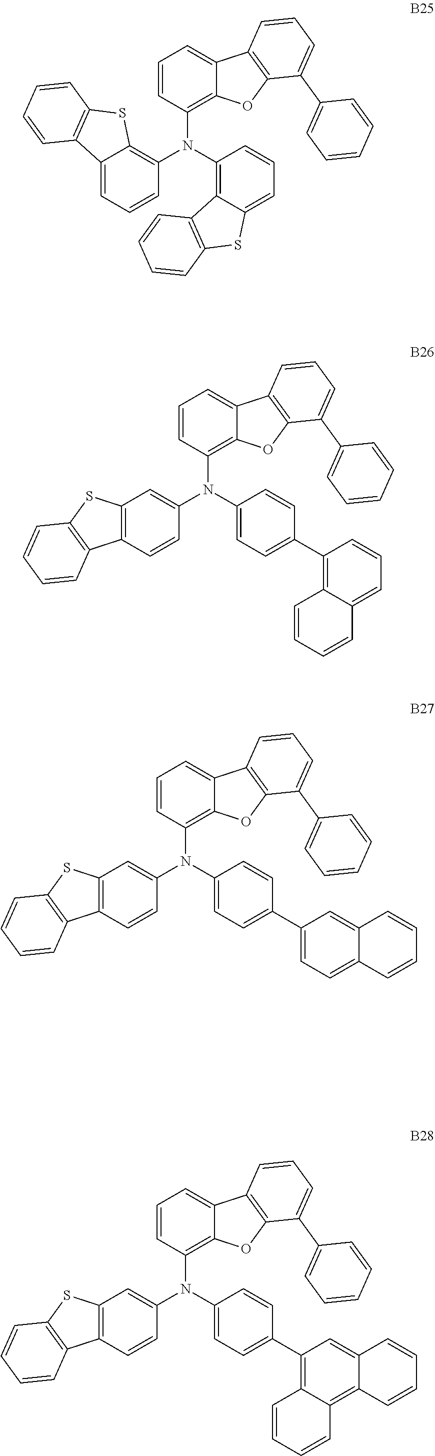 [SMILES: c1ccc(-c2cccc3c2oc2c(N(c4cccc5c4sc4ccccc45)c4cccc5sc6ccccc6c45)cccc23)cc1 ; c1ccc(-c2cccc3c2oc2c(N(c4ccc(-c5cccc6ccccc56)cc4)c4ccc5c(c4)sc4ccccc45)cccc23)cc1 ; c1ccc(-c2cccc3c2oc2c(N(c4ccc(-c5ccc6ccccc6c5)cc4)c4ccc5c(c4)sc4ccccc45)cccc23)cc1 ; c1ccc(-c2cccc3c2oc2c(N(c4ccc(-c5cc6ccccc6c6ccccc56)cc4)c4ccc5c(c4)sc4ccccc45)cccc23)cc1]

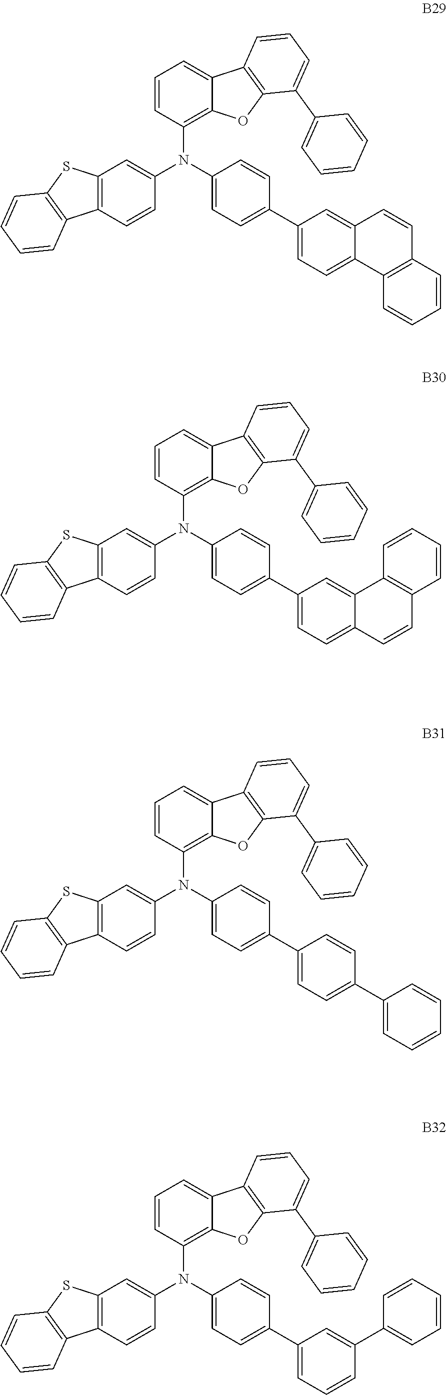 [SMILES: c1ccc(-c2cccc3c2oc2c(N(c4ccc(-c5ccc6c(ccc7ccccc76)c5)cc4)c4ccc5c(c4)sc4ccccc45)cccc23)cc1 ; c1ccc(-c2cccc3c2oc2c(N(c4ccc(-c5ccc6ccc7ccccc7c6c5)cc4)c4ccc5c(c4)sc4ccccc45)cccc23)cc1 ; c1ccc(-c2ccc(-c3ccc(N(c4ccc5c(c4)sc4ccccc45)c4cccc5c4oc4c(-c6ccccc6)cccc45)cc3)cc2)cc1 ; c1ccc(-c2cccc(-c3ccc(N(c4ccc5c(c4)sc4ccccc45)c4cccc5c4oc4c(-c6ccccc6)cccc45)cc3)c2)cc1]

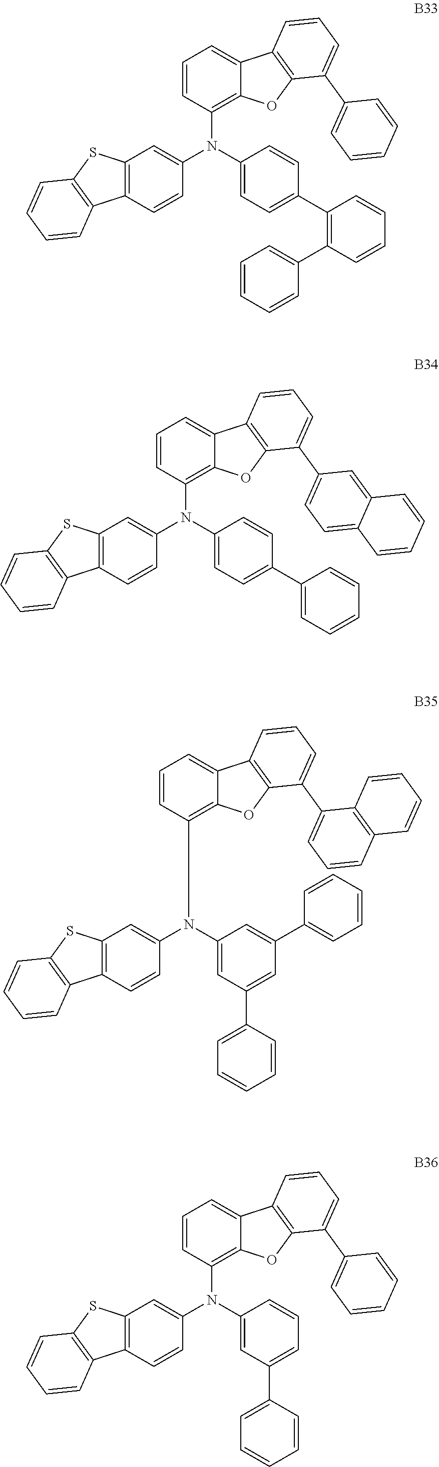 [SMILES: c1ccc(-c2ccccc2-c2ccc(N(c3ccc4c(c3)sc3ccccc34)c3cccc4c3oc3c(-c5ccccc5)cccc34)cc2)cc1 ; c1ccc(-c2ccc(N(c3ccc4c(c3)sc3ccccc34)c3cccc4c3oc3c(-c5ccc6ccccc6c5)cccc34)cc2)cc1 ; c1ccc(-c2cc(-c3ccccc3)cc(N(c3ccc4c(c3)sc3ccccc34)c3cccc4c3oc3c(-c5cccc6ccccc56)cccc34)c2)cc1 ; c1ccc(-c2cccc(N(c3ccc4c(c3)sc3ccccc34)c3cccc4c3oc3c(-c5ccccc5)cccc34)c2)cc1]

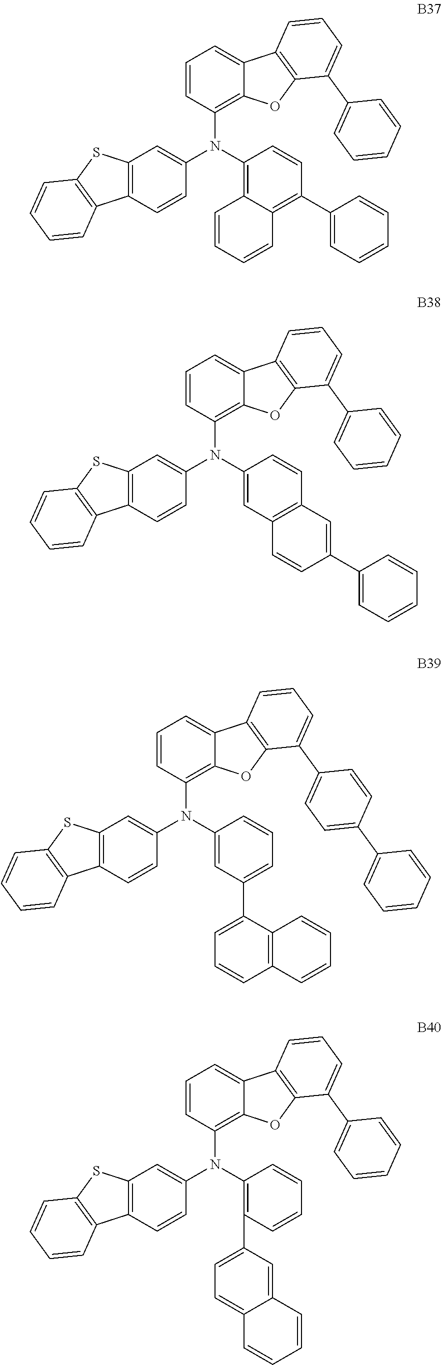 [SMILES: c1ccc(-c2ccc(N(c3ccc4c(c3)sc3ccccc34)c3cccc4c3oc3c(-c5ccccc5)cccc34)c3ccccc23)cc1 ; c1ccc(-c2ccc3cc(N(c4ccc5c(c4)sc4ccccc45)c4cccc5c4oc4c(-c6ccccc6)cccc45)ccc3c2)cc1 ; c1ccc(-c2ccc(-c3cccc4c3oc3c(N(c5cccc(-c6cccc7ccccc67)c5)c5ccc6c(c5)sc5ccccc56)cccc34)cc2)cc1 ; c1ccc(-c2cccc3c2oc2c(N(c4ccc5c(c4)sc4ccccc45)c4ccccc4-c4ccc5ccccc5c4)cccc23)cc1]

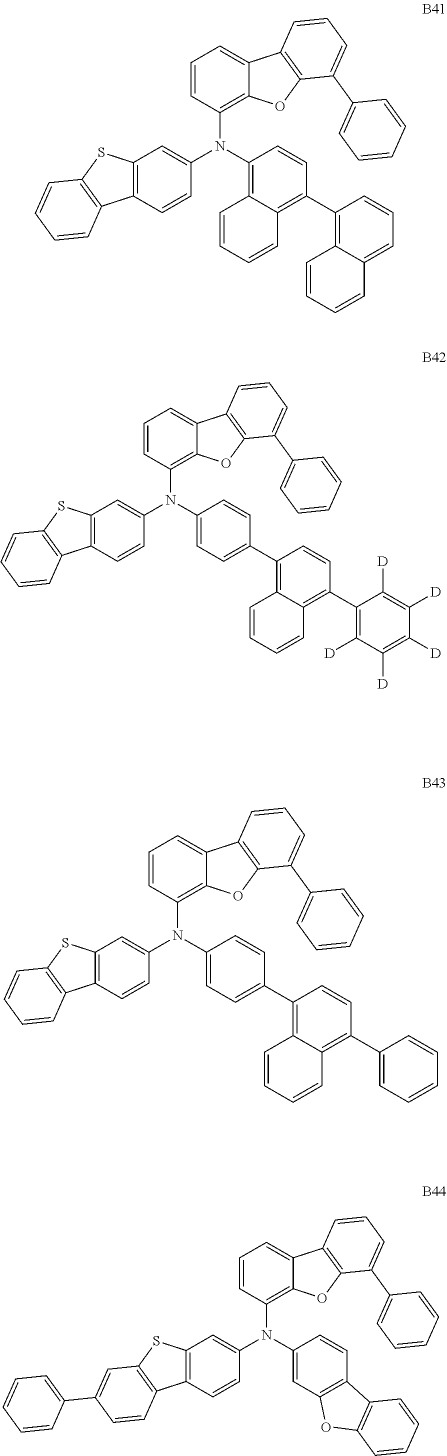 [SMILES: c1ccc(-c2cccc3c2oc2c(N(c4ccc5c(c4)sc4ccccc45)c4ccc(-c5cccc6ccccc56)c5ccccc45)cccc23)cc1 ; [2H]c1c([2H])c([2H])c(-c2ccc(-c3ccc(N(c4ccc5c(c4)sc4ccccc45)c4cccc5c4oc4c(-c6ccccc6)cccc45)cc3)c3ccccc23)c([2H])c1[2H] ; c1ccc(-c2ccc(-c3ccc(N(c4ccc5c(c4)sc4ccccc45)c4cccc5c4oc4c(-c6ccccc6)cccc45)cc3)c3ccccc23)cc1 ; c1ccc(-c2ccc3c(c2)sc2cc(N(c4ccc5c(c4)oc4ccccc45)c4cccc5c4oc4c(-c6ccccc6)cccc45)ccc23)cc1]

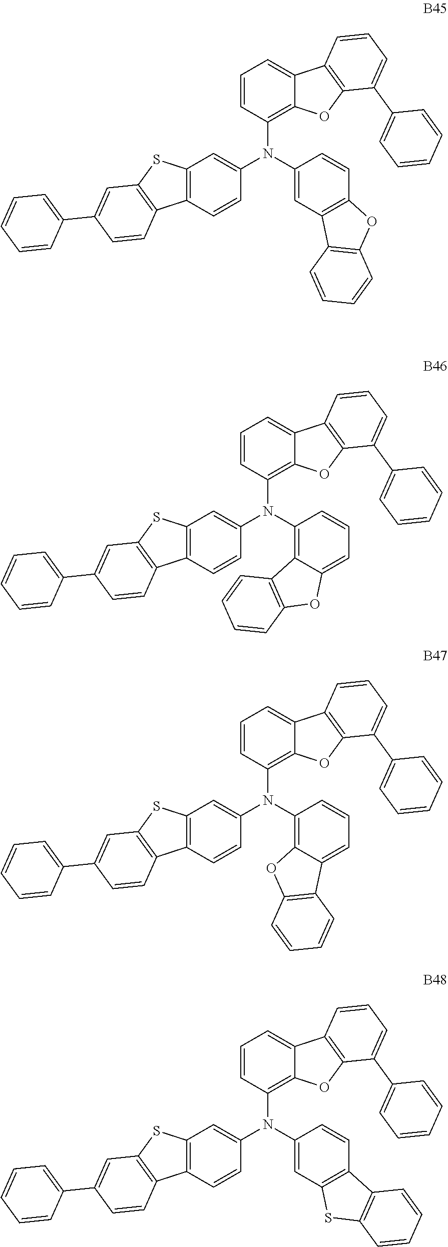 [SMILES: c1ccc(-c2ccc3c(c2)sc2cc(N(c4ccc5oc6ccccc6c5c4)c4cccc5c4oc4c(-c6ccccc6)cccc45)ccc23)cc1 ; c1ccc(-c2ccc3c(c2)sc2cc(N(c4cccc5c4oc4c(-c6ccccc6)cccc45)c4cccc5oc6ccccc6c45)ccc23)cc1 ; c1ccc(-c2ccc3c(c2)sc2cc(N(c4cccc5c4oc4ccccc45)c4cccc5c4oc4c(-c6ccccc6)cccc45)ccc23)cc1 ; c1ccc(-c2ccc3c(c2)sc2cc(N(c4ccc5c(c4)sc4ccccc45)c4cccc5c4oc4c(-c6ccccc6)cccc45)ccc23)cc1]

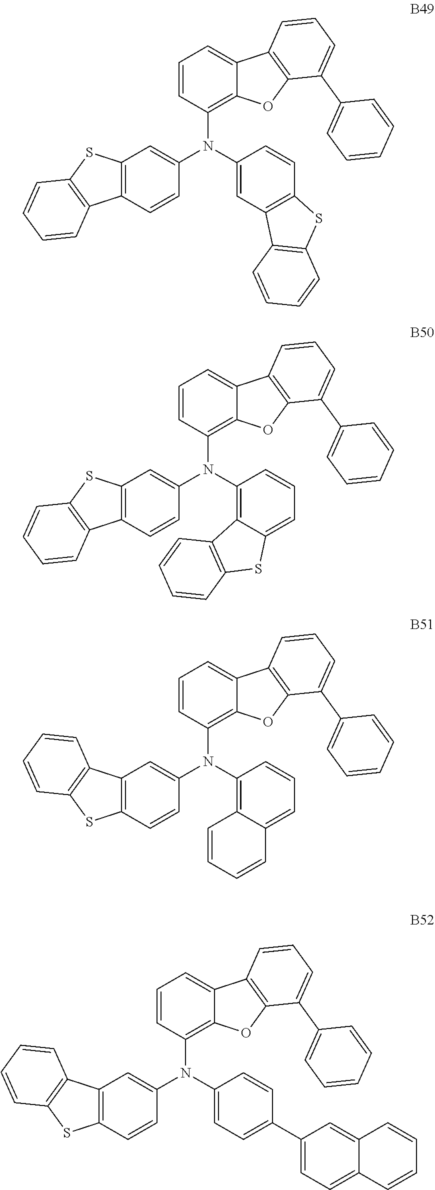

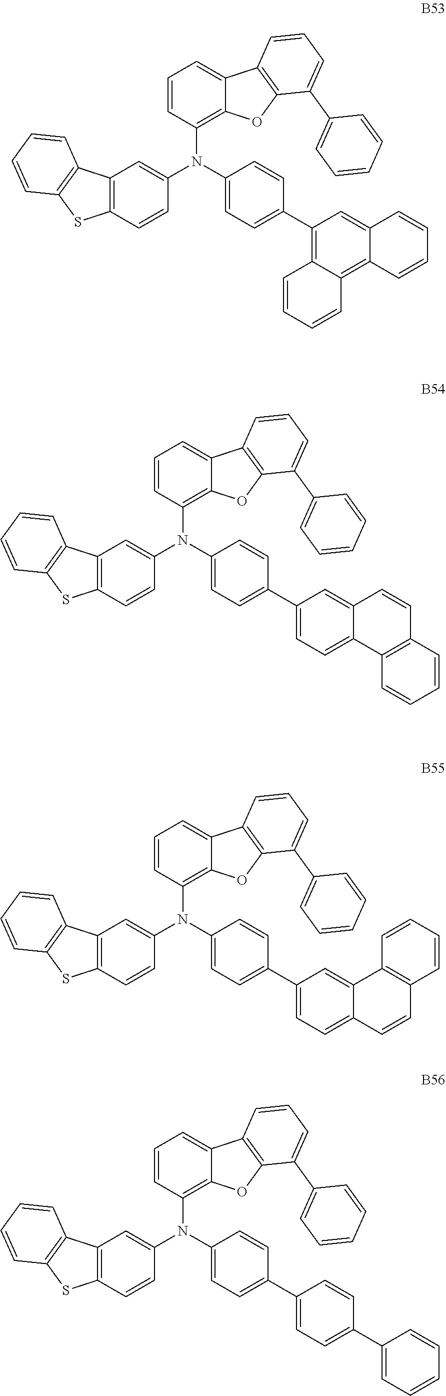 [SMILES: c1ccc(-c2cccc3c2oc2c(N(c4ccc(-c5cc6ccccc6c6ccccc56)cc4)c4ccc5sc6ccccc6c5c4)cccc23)cc1 ; c1ccc(-c2cccc3c2oc2c(N(c4ccc(-c5ccc6c(ccc7ccccc76)c5)cc4)c4ccc5sc6ccccc6c5c4)cccc23)cc1 ; c1ccc(-c2cccc3c2oc2c(N(c4ccc(-c5ccc6ccc7ccccc7c6c5)cc4)c4ccc5sc6ccccc6c5c4)cccc23)cc1 ; c1ccc(-c2ccc(-c3ccc(N(c4ccc5sc6ccccc6c5c4)c4cccc5c4oc4c(-c6ccccc6)cccc45)cc3)cc2)cc1]

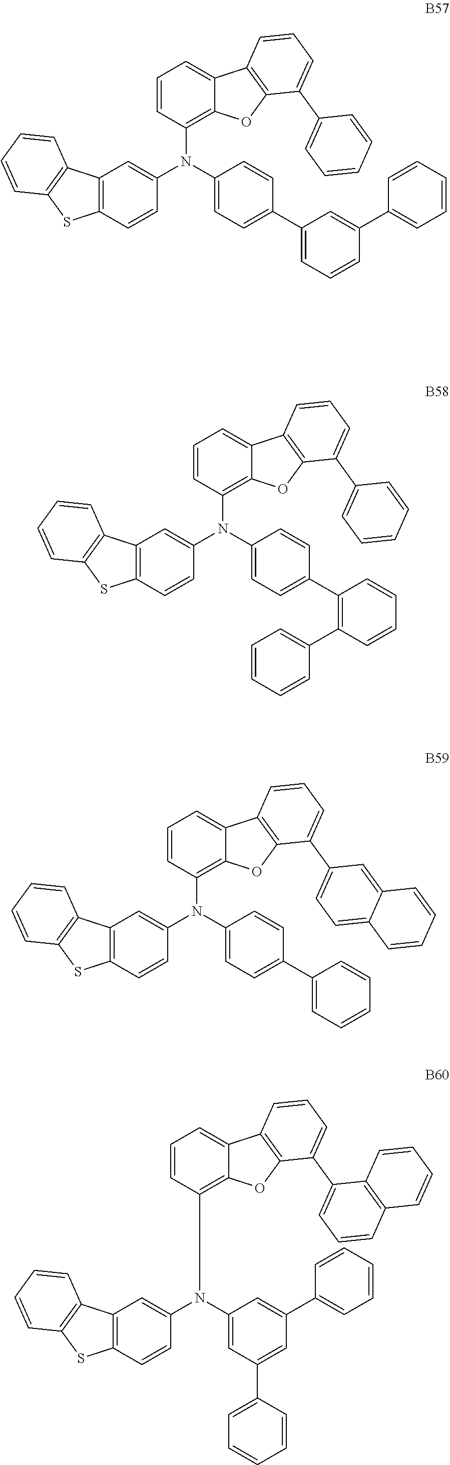 [SMILES: c1ccc(-c2cccc(-c3ccc(N(c4ccc5sc6ccccc6c5c4)c4cccc5c4oc4c(-c6ccccc6)cccc45)cc3)c2)cc1 ; c1ccc(-c2ccccc2-c2ccc(N(c3ccc4sc5ccccc5c4c3)c3cccc4c3oc3c(-c5ccccc5)cccc34)cc2)cc1 ; c1ccc(-c2ccc(N(c3ccc4sc5ccccc5c4c3)c3cccc4c3oc3c(-c5ccc6ccccc6c5)cccc34)cc2)cc1 ; c1ccc(-c2cc(-c3ccccc3)cc(N(c3ccc4sc5ccccc5c4c3)c3cccc4c3oc3c(-c5cccc6ccccc56)cccc34)c2)cc1]

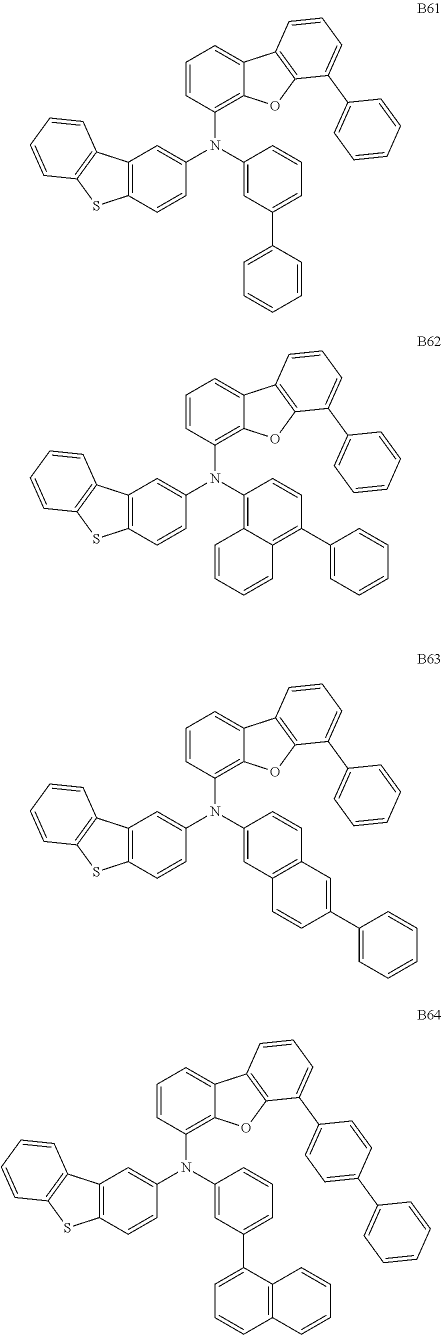

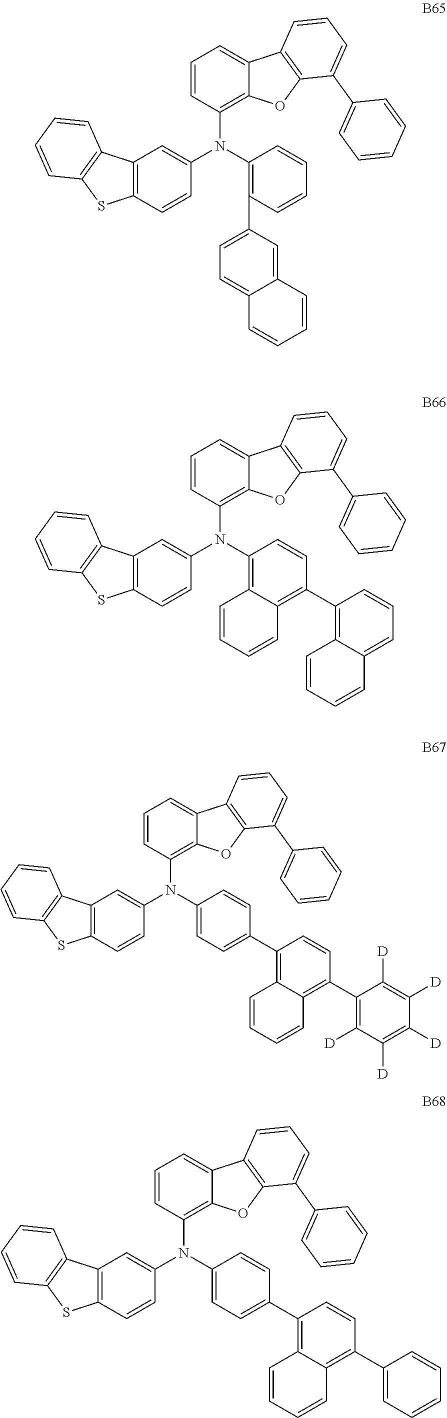 [SMILES: c1ccc(-c2cccc3c2oc2c(N(c4ccc5sc6ccccc6c5c4)c4ccccc4-c4ccc5ccccc5c4)cccc23)cc1 ; c1ccc(-c2cccc3c2oc2c(N(c4ccc5sc6ccccc6c5c4)c4ccc(-c5cccc6ccccc56)c5ccccc45)cccc23)cc1 ; [2H]c1c([2H])c([2H])c(-c2ccc(-c3ccc(N(c4ccc5sc6ccccc6c5c4)c4cccc5c4oc4c(-c6ccccc6)cccc45)cc3)c3ccccc23)c([2H])c1[2H] ; c1ccc(-c2ccc(-c3ccc(N(c4ccc5sc6ccccc6c5c4)c4cccc5c4oc4c(-c6ccccc6)cccc45)cc3)c3ccccc23)cc1]

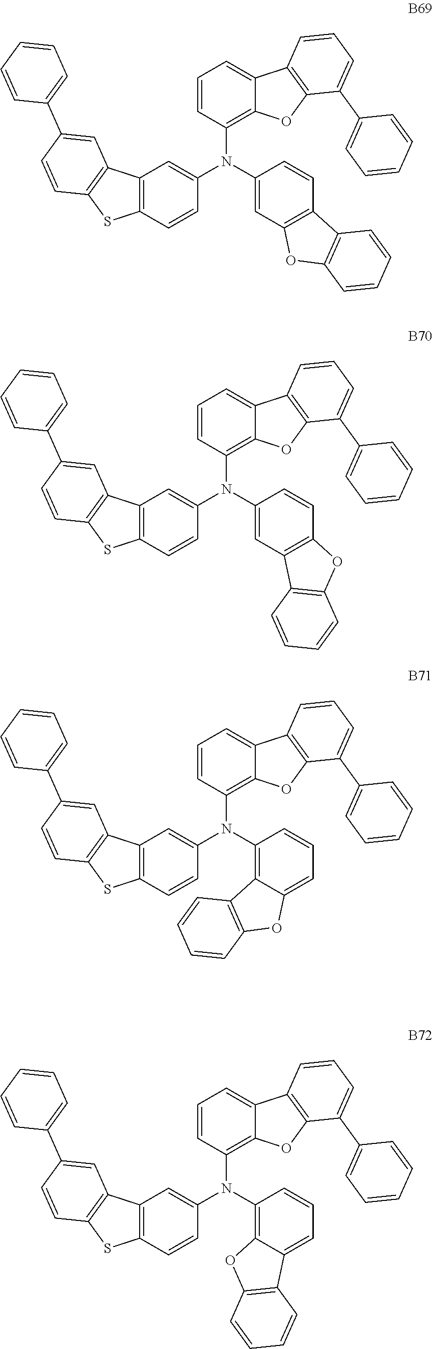 [SMILES: c1ccc(-c2ccc3sc4ccc(N(c5ccc6c(c5)oc5ccccc56)c5cccc6c5oc5c(-c7ccccc7)cccc56)cc4c3c2)cc1 ; c1ccc(-c2ccc3sc4ccc(N(c5ccc6oc7ccccc7c6c5)c5cccc6c5oc5c(-c7ccccc7)cccc56)cc4c3c2)cc1 ; c1ccc(-c2ccc3sc4ccc(N(c5cccc6c5oc5c(-c7ccccc7)cccc56)c5cccc6oc7ccccc7c56)cc4c3c2)cc1 ; c1ccc(-c2ccc3sc4ccc(N(c5cccc6c5oc5ccccc56)c5cccc6c5oc5c(-c7ccccc7)cccc56)cc4c3c2)cc1]

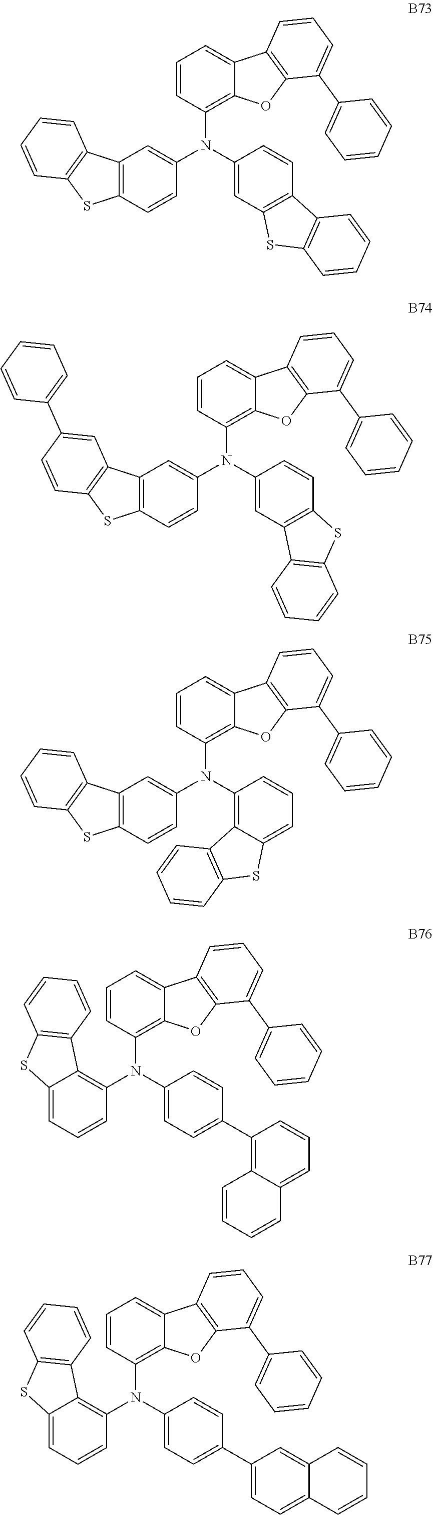

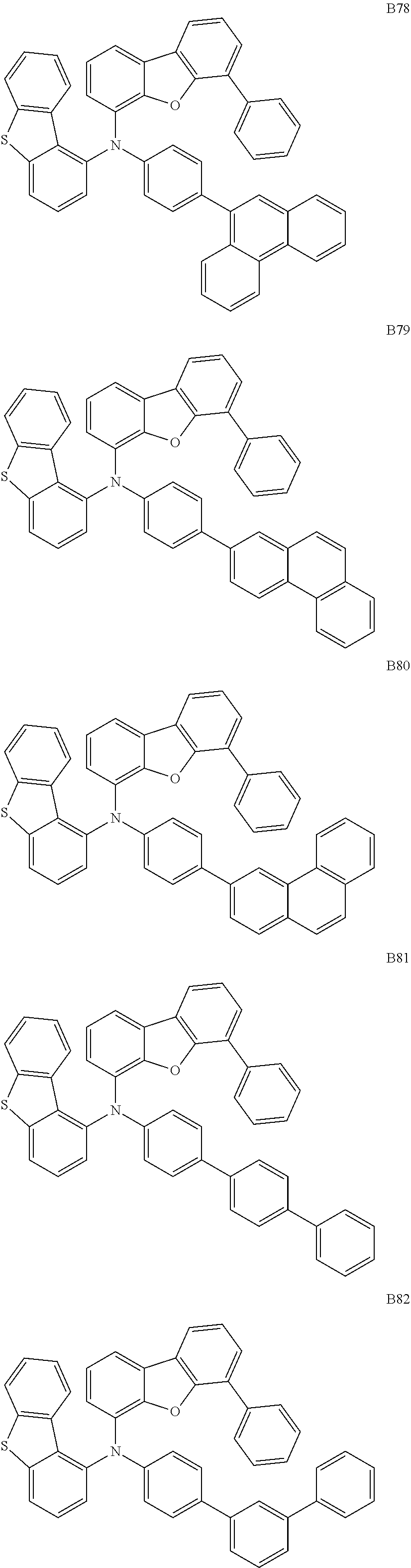

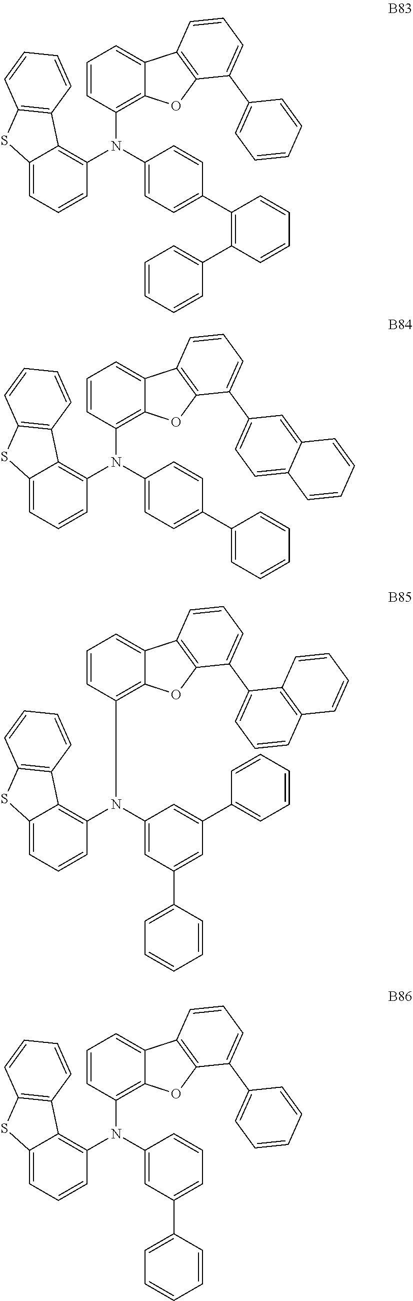

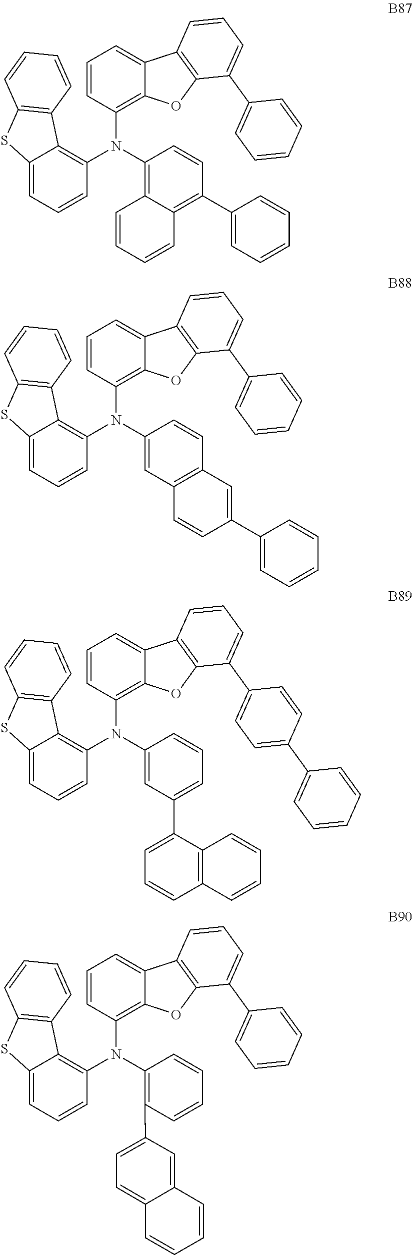

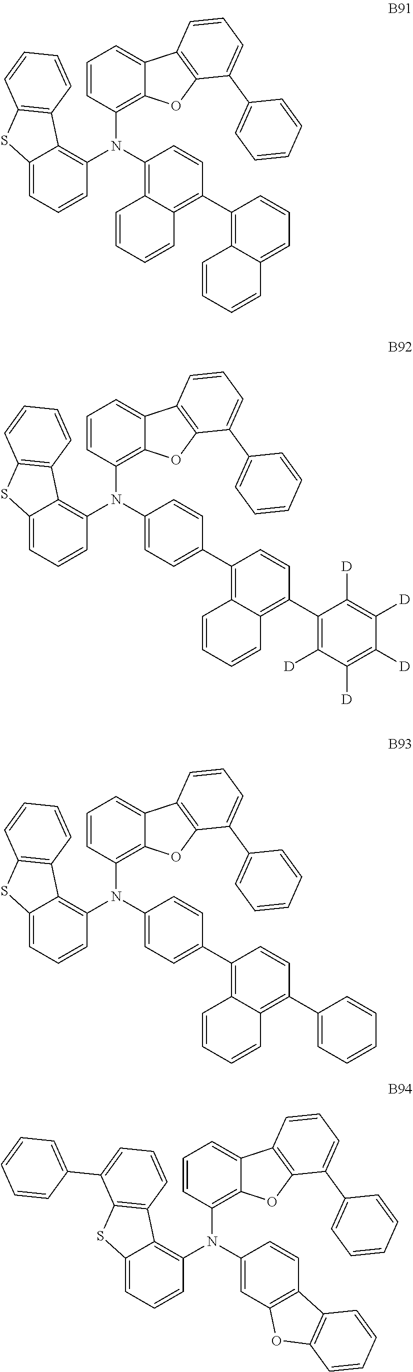 [SMILES: c1ccc(-c2cccc3c2oc2c(N(c4ccc(-c5cccc6ccccc56)c5ccccc45)c4cccc5sc6ccccc6c45)cccc23)cc1 ; [2H]c1c([2H])c([2H])c(-c2ccc(-c3ccc(N(c4cccc5c4oc4c(-c6ccccc6)cccc45)c4cccc5sc6ccccc6c45)cc3)c3ccccc23)c([2H])c1[2H] ; c1ccc(-c2ccc(-c3ccc(N(c4cccc5c4oc4c(-c6ccccc6)cccc45)c4cccc5sc6ccccc6c45)cc3)c3ccccc23)cc1 ; c1ccc(-c2cccc3c2oc2c(N(c4ccc5c(c4)oc4ccccc45)c4cccc5sc6c(-c7ccccc7)cccc6c45)cccc23)cc1]

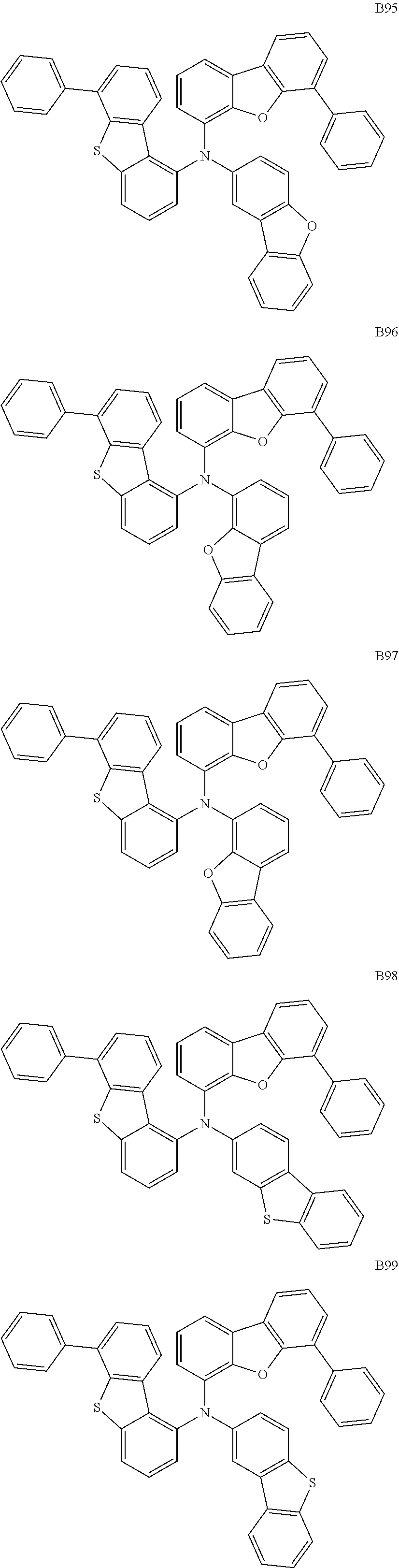

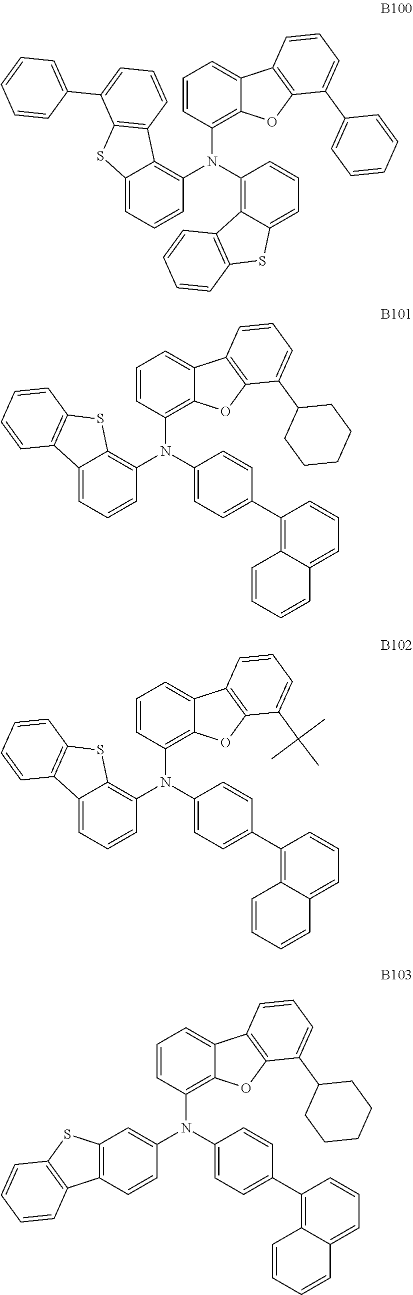

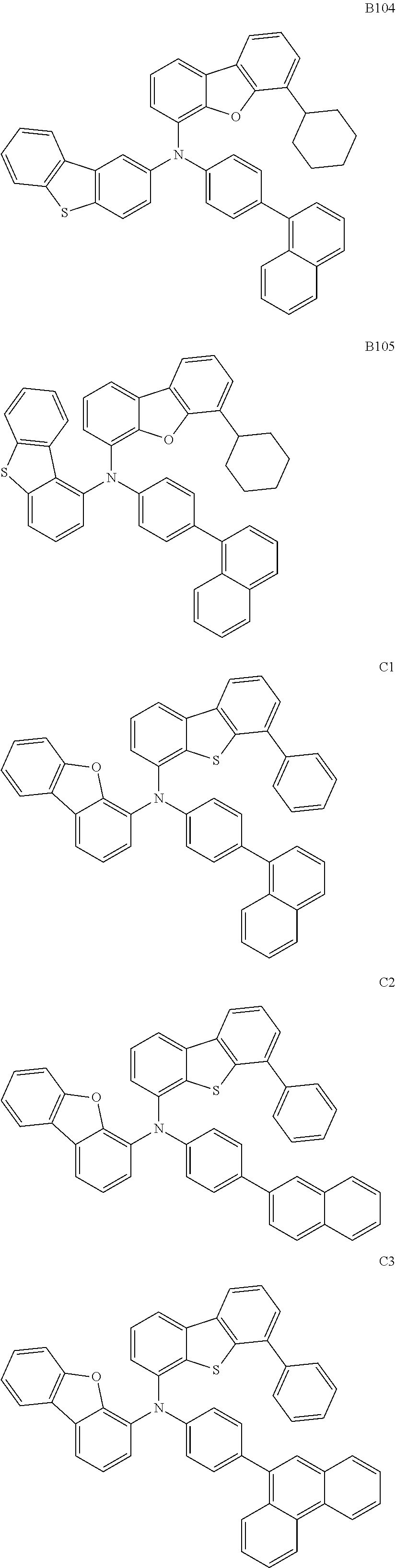

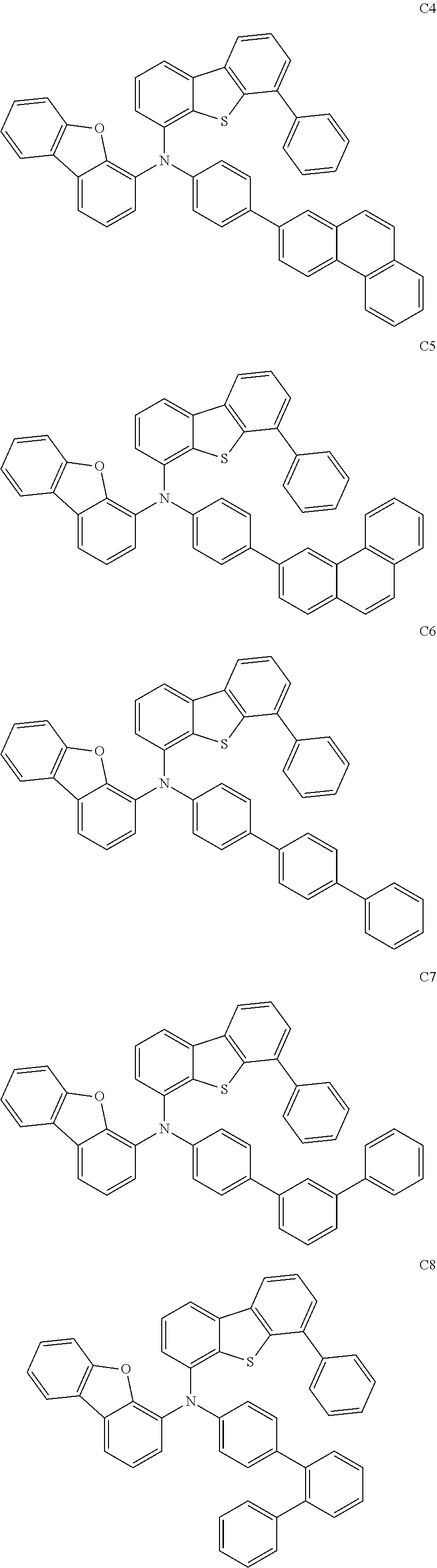

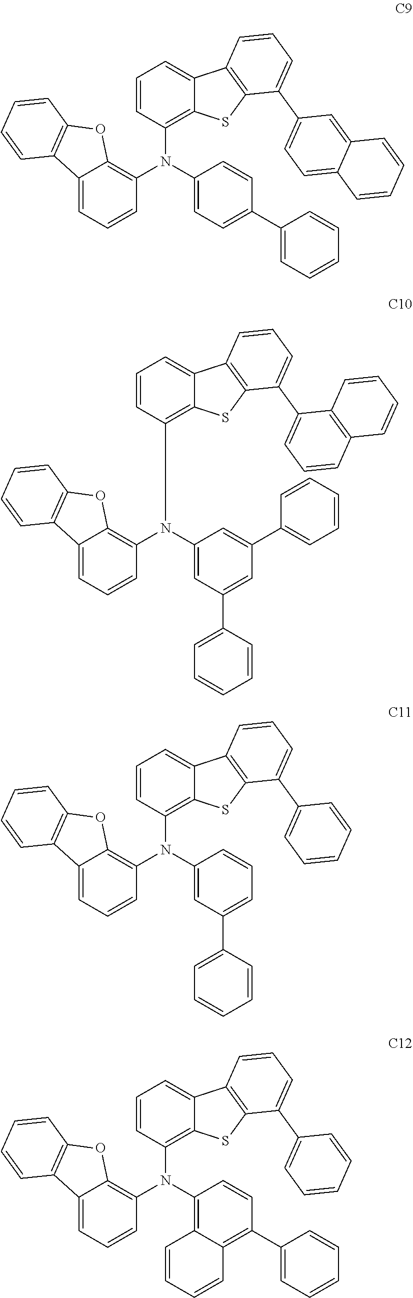

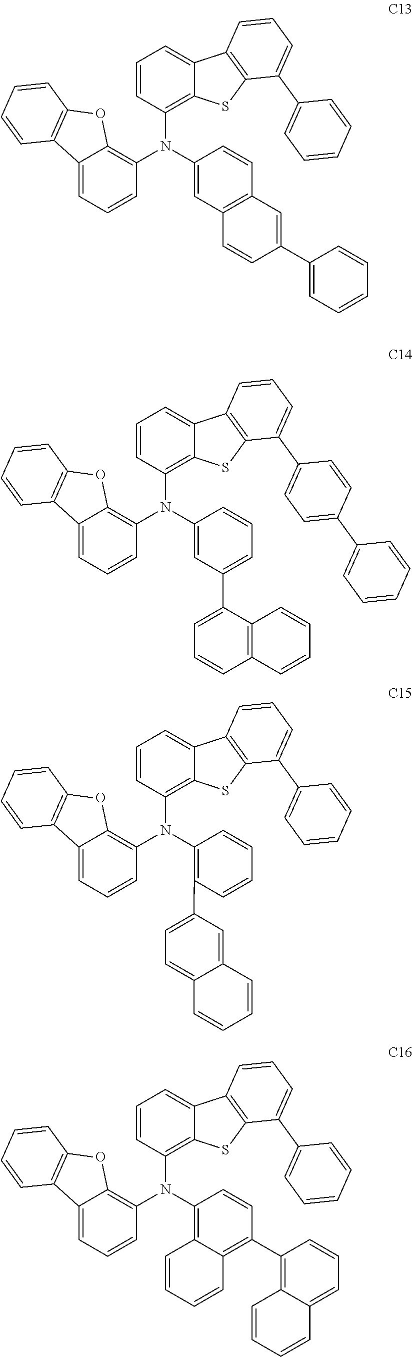

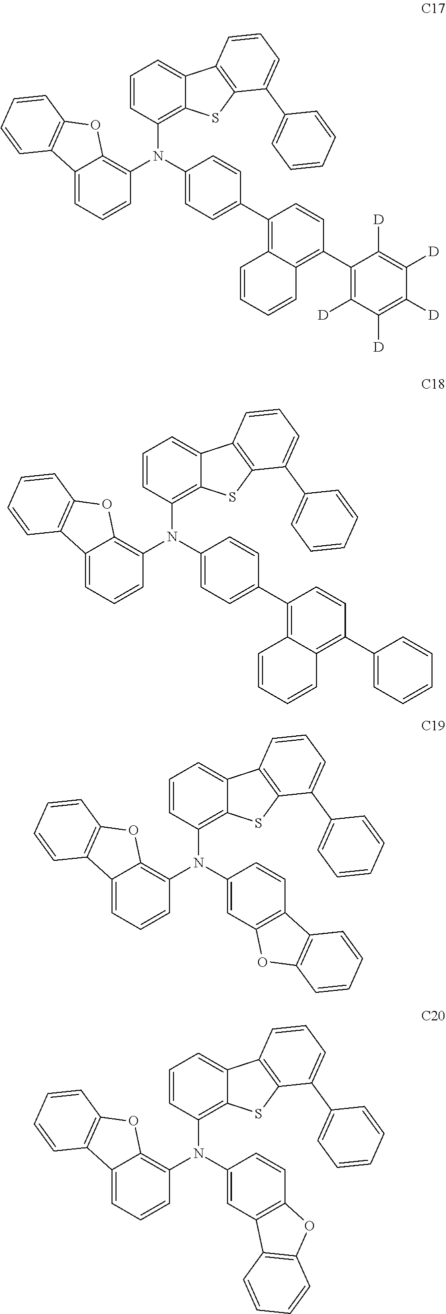

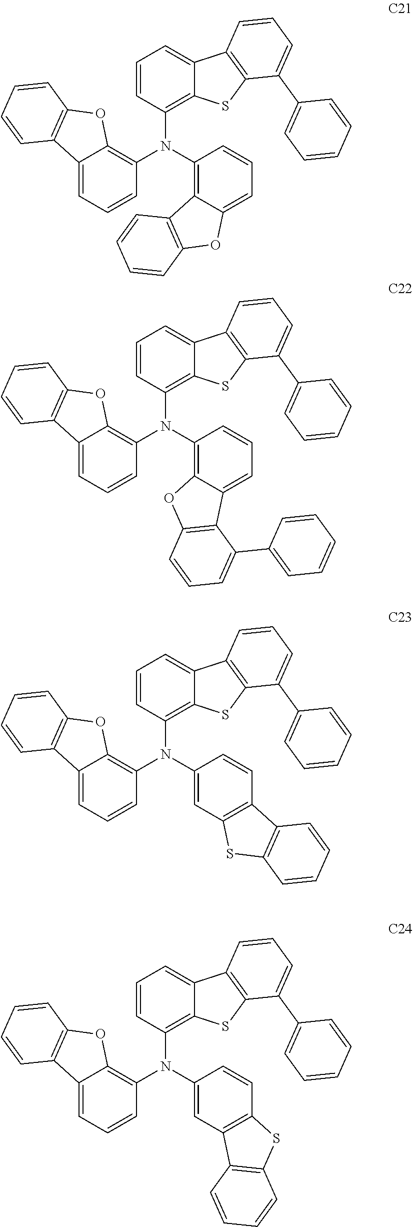 [SMILES: c1ccc(-c2cccc3c2sc2c(N(c4cccc5c4oc4ccccc45)c4cccc5oc6ccccc6c45)cccc23)cc1 ; c1ccc(-c2cccc3c2sc2c(N(c4cccc5c4oc4ccccc45)c4cccc5c4oc4cccc(-c6ccccc6)c45)cccc23)cc1 ; c1ccc(-c2cccc3c2sc2c(N(c4ccc5c(c4)sc4ccccc45)c4cccc5c4oc4ccccc45)cccc23)cc1 ; c1ccc(-c2cccc3c2sc2c(N(c4ccc5sc6ccccc6c5c4)c4cccc5c4oc4ccccc45)cccc23)cc1]

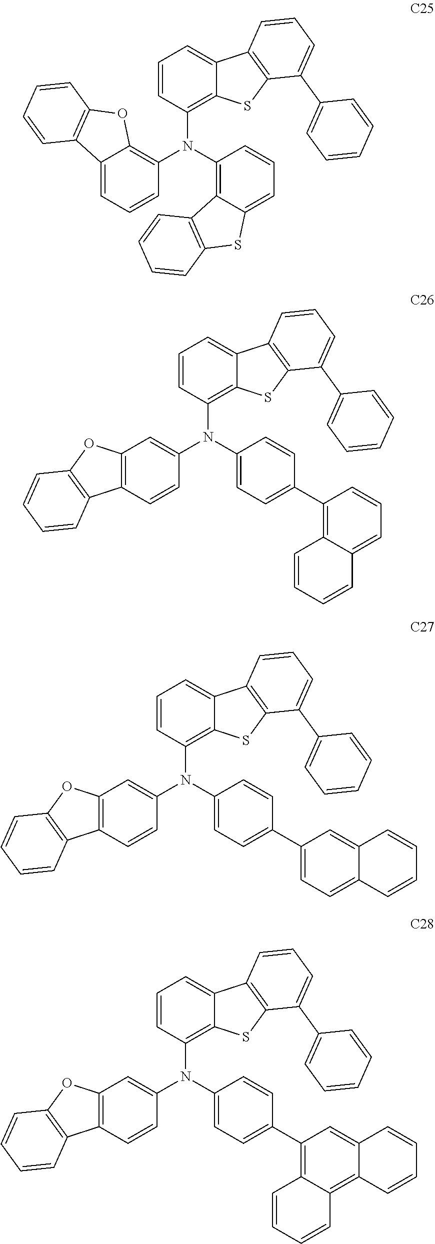 [SMILES: c1ccc(-c2cccc3c2sc2c(N(c4cccc5c4oc4ccccc45)c4cccc5sc6ccccc6c45)cccc23)cc1 ; c1ccc(-c2cccc3c2sc2c(N(c4ccc(-c5cccc6ccccc56)cc4)c4ccc5c(c4)oc4ccccc45)cccc23)cc1 ; c1ccc(-c2cccc3c2sc2c(N(c4ccc(-c5ccc6ccccc6c5)cc4)c4ccc5c(c4)oc4ccccc45)cccc23)cc1 ; c1ccc(-c2cccc3c2sc2c(N(c4ccc(-c5cc6ccccc6c6ccccc56)cc4)c4ccc5c(c4)oc4ccccc45)cccc23)cc1]

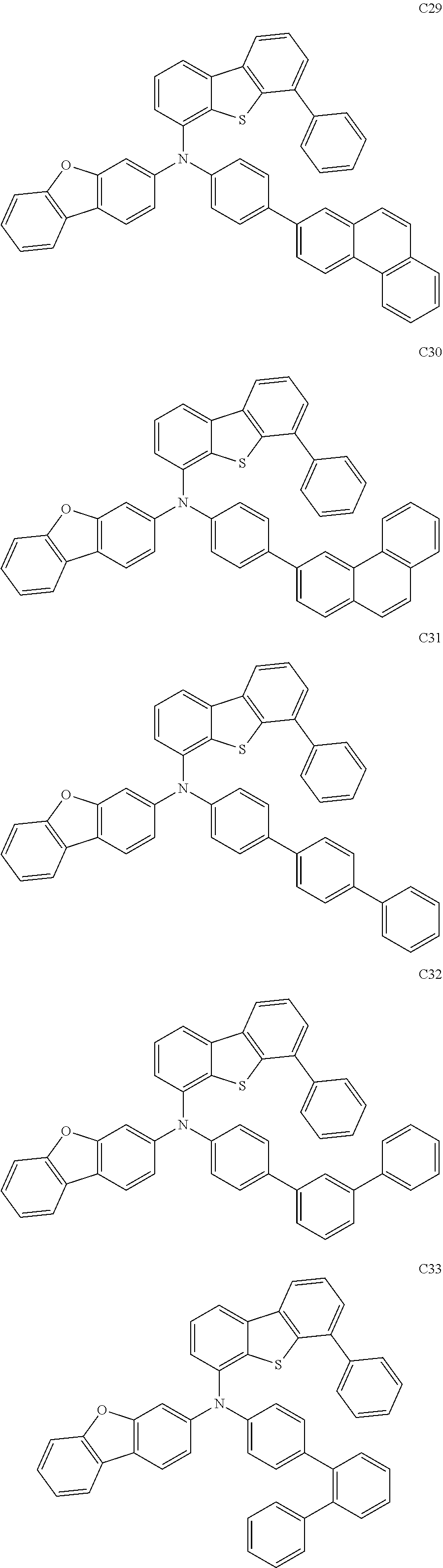

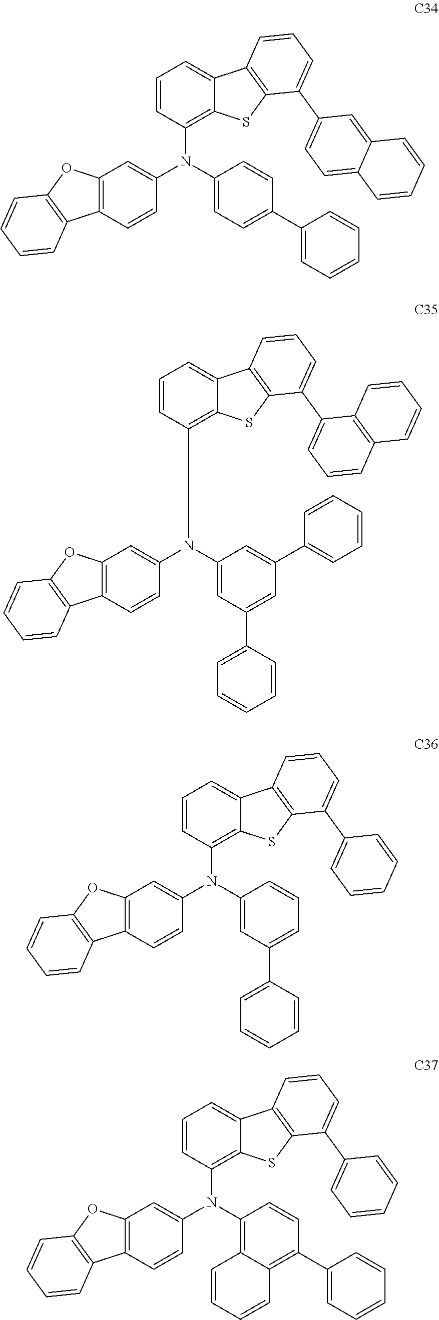

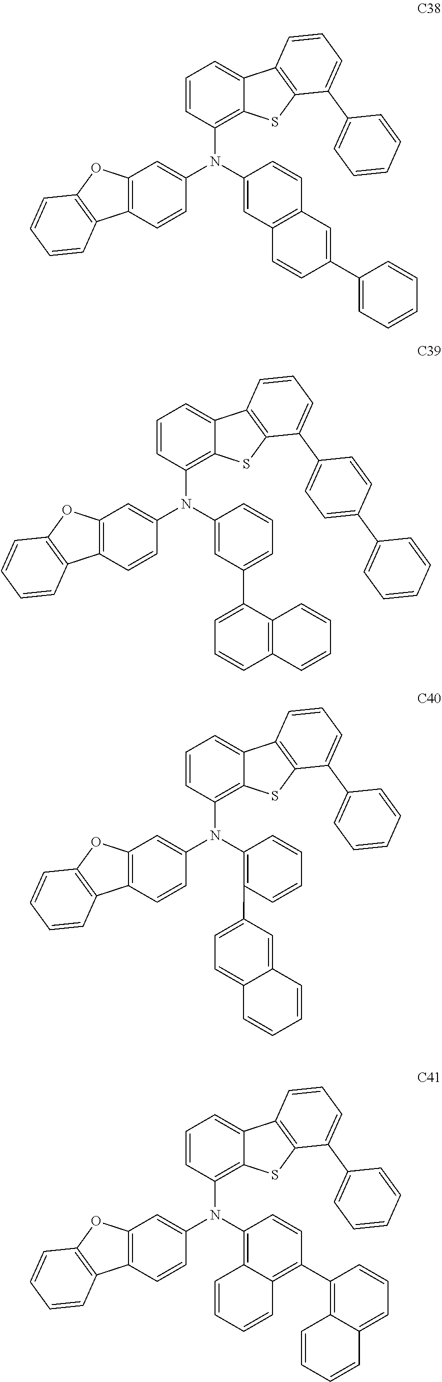

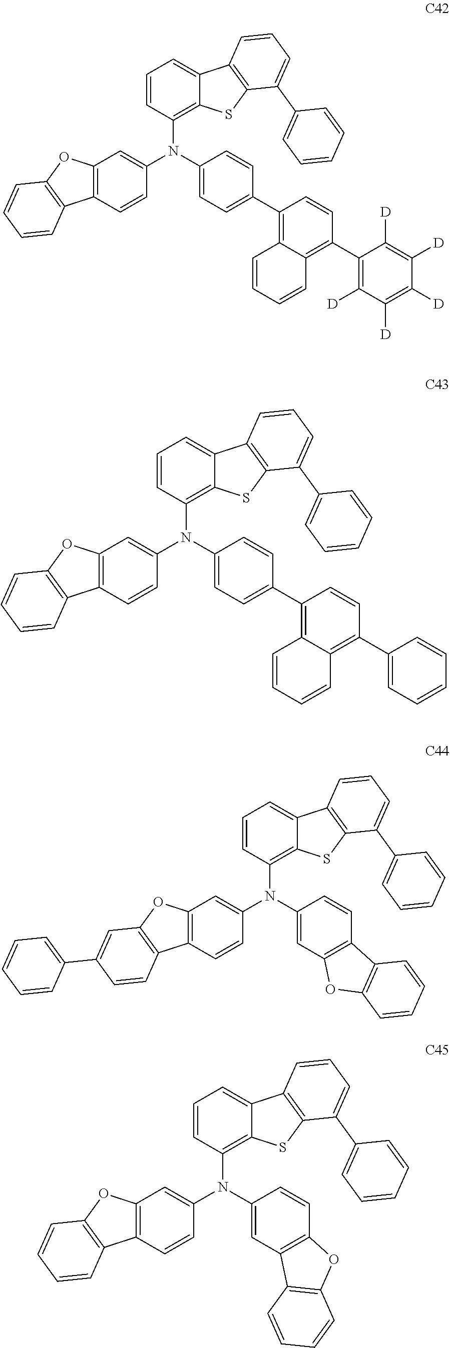 [SMILES: [2H]c1c([2H])c([2H])c(-c2ccc(-c3ccc(N(c4ccc5c(c4)oc4ccccc45)c4cccc5c4sc4c(-c6ccccc6)cccc45)cc3)c3ccccc23)c([2H])c1[2H] ; c1ccc(-c2ccc(-c3ccc(N(c4ccc5c(c4)oc4ccccc45)c4cccc5c4sc4c(-c6ccccc6)cccc45)cc3)c3ccccc23)cc1 ; c1ccc(-c2ccc3c(c2)oc2cc(N(c4ccc5c(c4)oc4ccccc45)c4cccc5c4sc4c(-c6ccccc6)cccc45)ccc23)cc1 ; c1ccc(-c2cccc3c2sc2c(N(c4ccc5c(c4)oc4ccccc45)c4ccc5oc6ccccc6c5c4)cccc23)cc1]

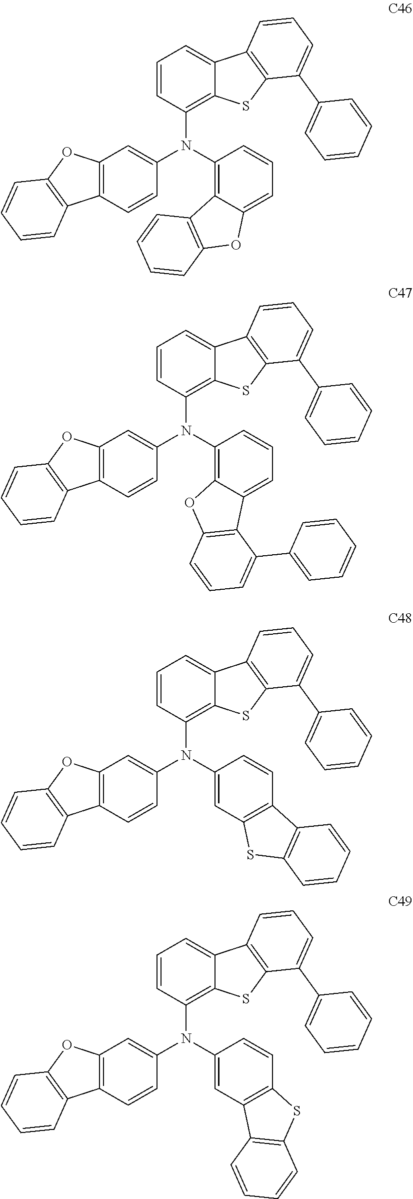

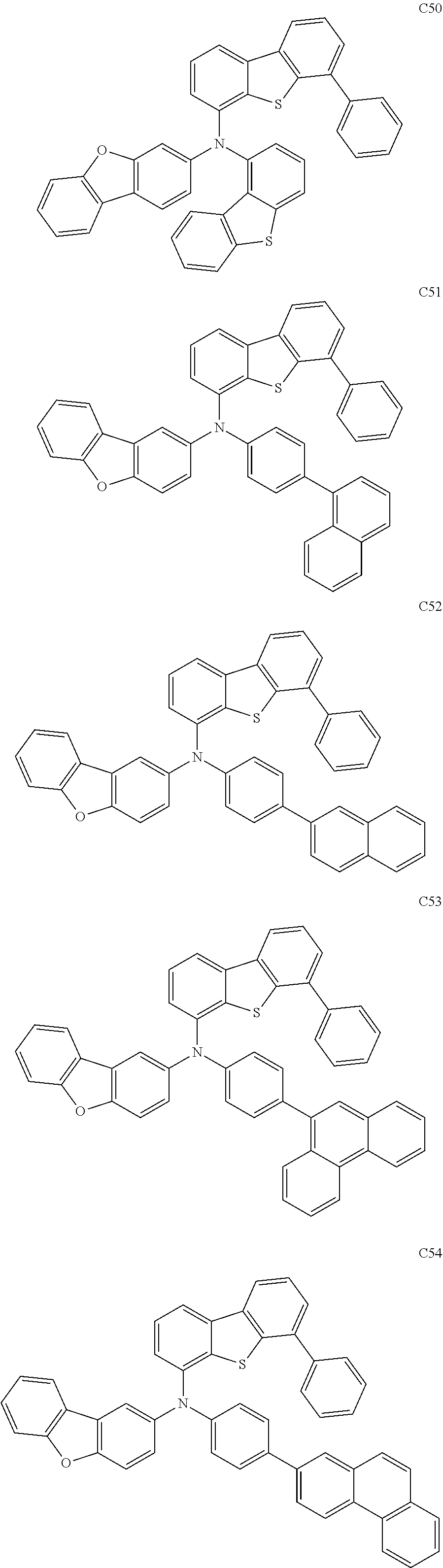 [SMILES: c1ccc(-c2cccc3c2sc2c(N(c4ccc5c(c4)oc4ccccc45)c4cccc5sc6ccccc6c45)cccc23)cc1 ; c1ccc(-c2cccc3c2sc2c(N(c4ccc(-c5cccc6ccccc56)cc4)c4ccc5oc6ccccc6c5c4)cccc23)cc1 ; c1ccc(-c2cccc3c2sc2c(N(c4ccc(-c5ccc6ccccc6c5)cc4)c4ccc5oc6ccccc6c5c4)cccc23)cc1 ; c1ccc(-c2cccc3c2sc2c(N(c4ccc(-c5cc6ccccc6c6ccccc56)cc4)c4ccc5oc6ccccc6c5c4)cccc23)cc1 ; c1ccc(-c2cccc3c2sc2c(N(c4ccc(-c5ccc6c(ccc7ccccc76)c5)cc4)c4ccc5oc6ccccc6c5c4)cccc23)cc1]

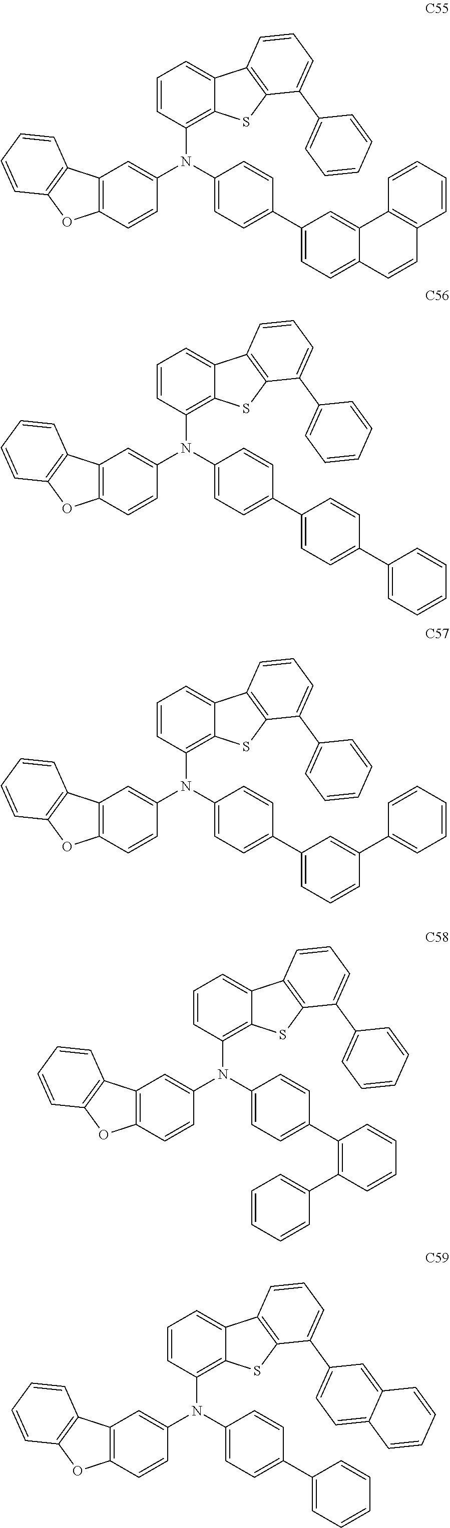

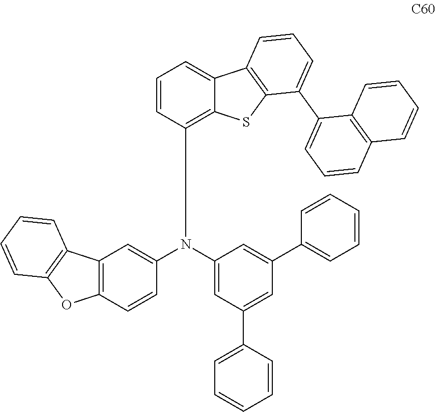 [SMILES: c1ccc(-c2cc(-c3ccccc3)cc(N(c3ccc4oc5ccccc5c4c3)c3cccc4c3sc3c(-c5cccc6ccccc56)cccc34)c2)cc1]

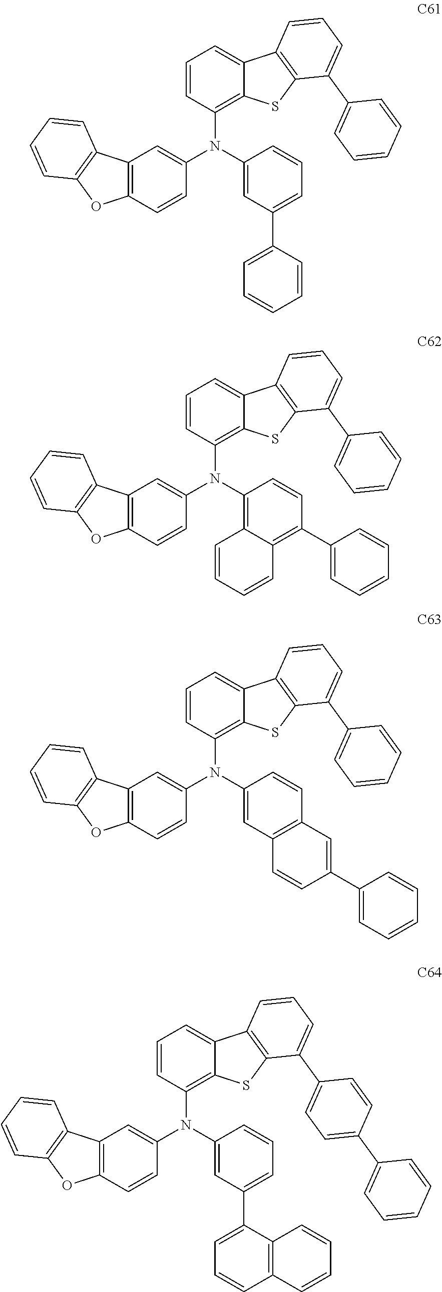

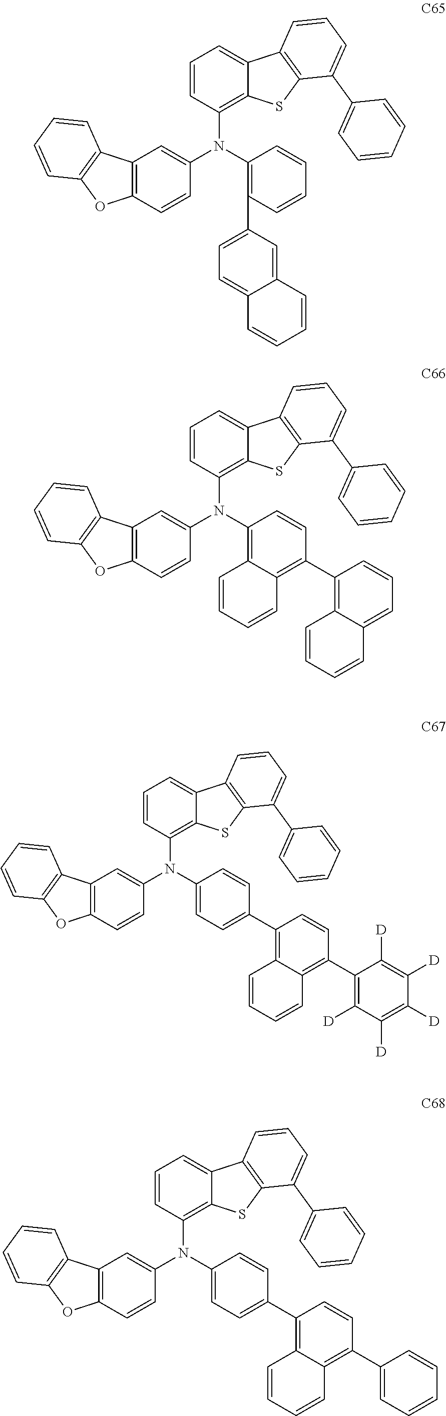

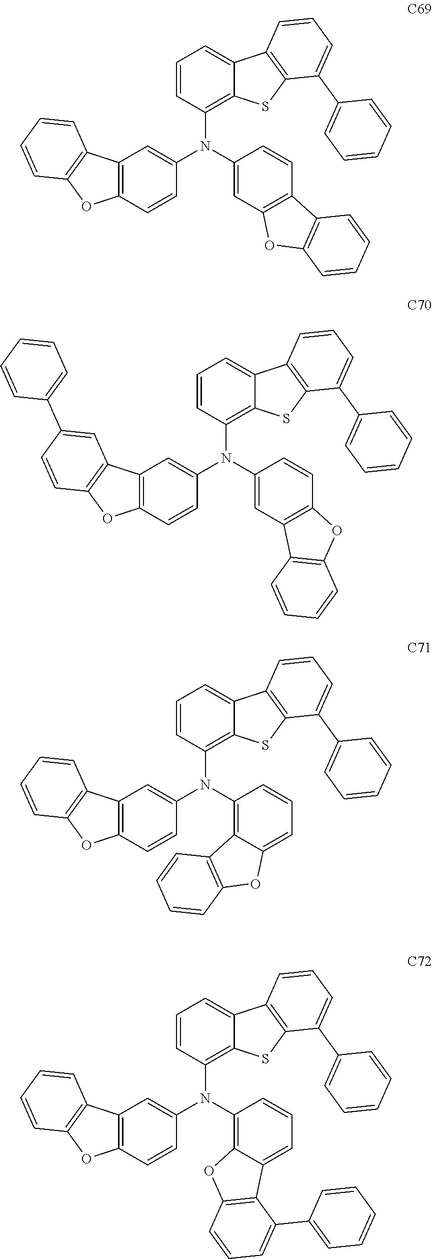

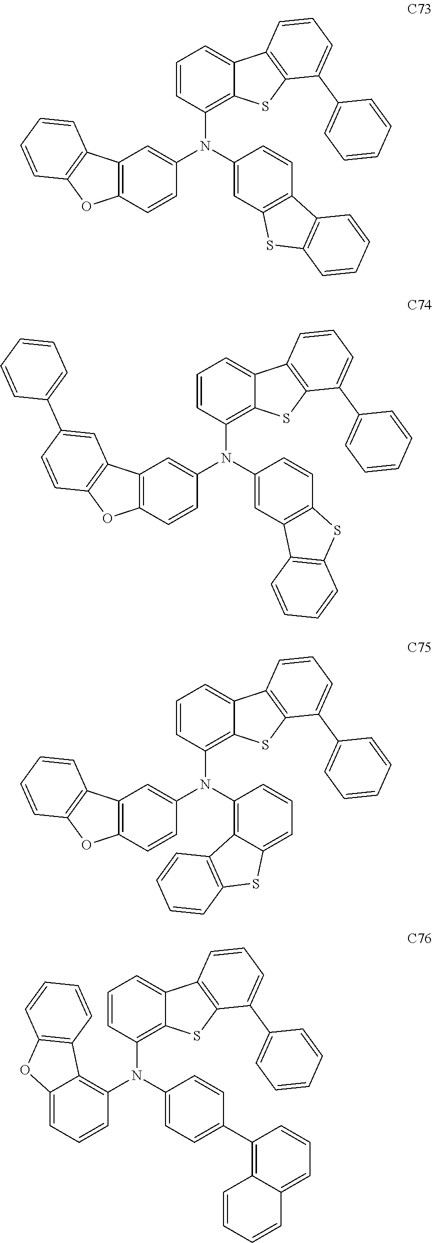

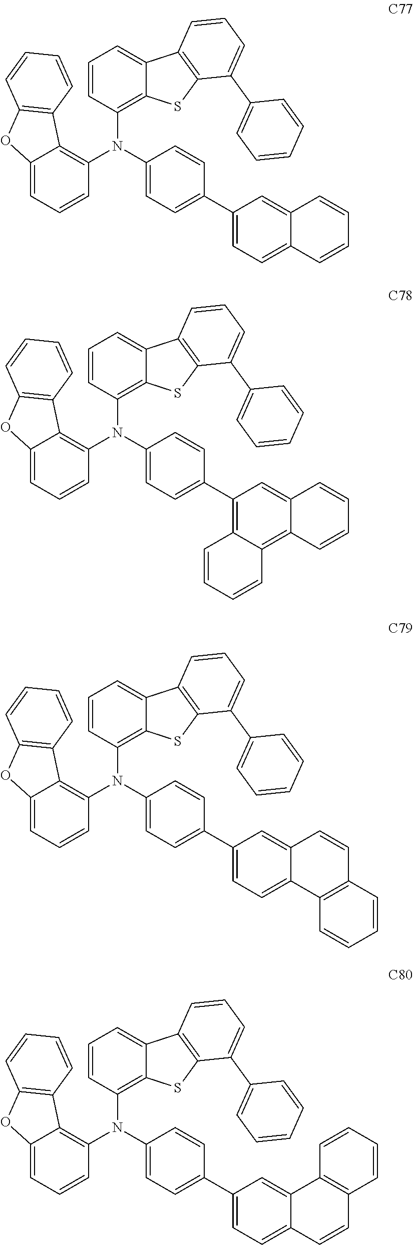 [SMILES: c1ccc(-c2cccc3c2sc2c(N(c4ccc(-c5ccc6ccccc6c5)cc4)c4cccc5oc6ccccc6c45)cccc23)cc1 ; c1ccc(-c2cccc3c2sc2c(N(c4ccc(-c5cc6ccccc6c6ccccc56)cc4)c4cccc5oc6ccccc6c45)cccc23)cc1 ; c1ccc(-c2cccc3c2sc2c(N(c4ccc(-c5ccc6c(ccc7ccccc76)c5)cc4)c4cccc5oc6ccccc6c45)cccc23)cc1 ; c1ccc(-c2cccc3c2sc2c(N(c4ccc(-c5ccc6ccc7ccccc7c6c5)cc4)c4cccc5oc6ccccc6c45)cccc23)cc1]

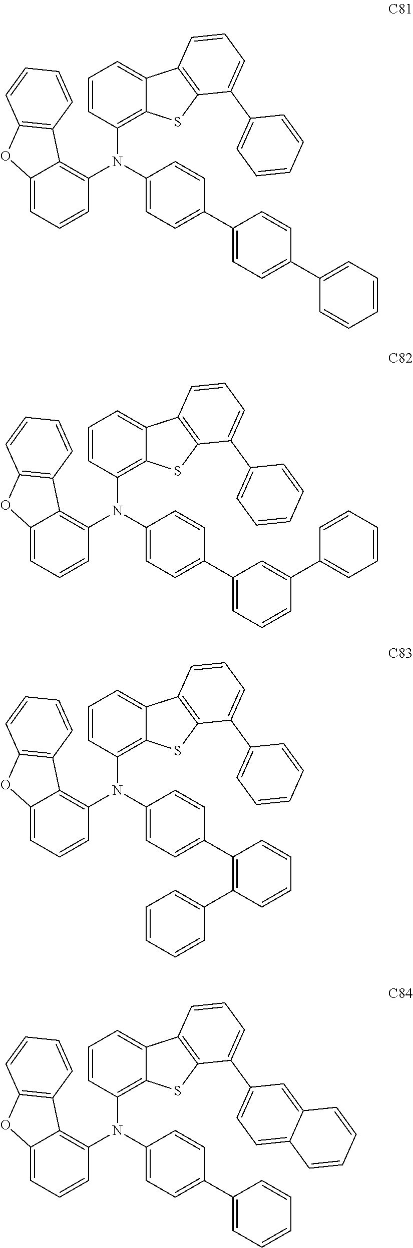 [SMILES: c1ccc(-c2ccc(-c3ccc(N(c4cccc5c4sc4c(-c6ccccc6)cccc45)c4cccc5oc6ccccc6c45)cc3)cc2)cc1 ; c1ccc(-c2cccc(-c3ccc(N(c4cccc5c4sc4c(-c6ccccc6)cccc45)c4cccc5oc6ccccc6c45)cc3)c2)cc1 ; c1ccc(-c2ccccc2-c2ccc(N(c3cccc4c3sc3c(-c5ccccc5)cccc34)c3cccc4oc5ccccc5c34)cc2)cc1 ; c1ccc(-c2ccc(N(c3cccc4c3sc3c(-c5ccc6ccccc6c5)cccc34)c3cccc4oc5ccccc5c34)cc2)cc1]

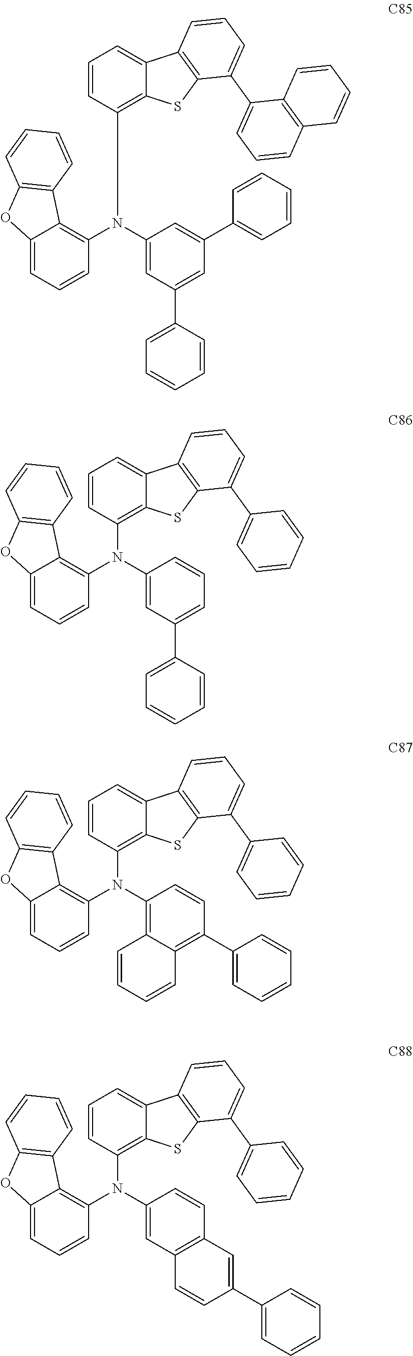

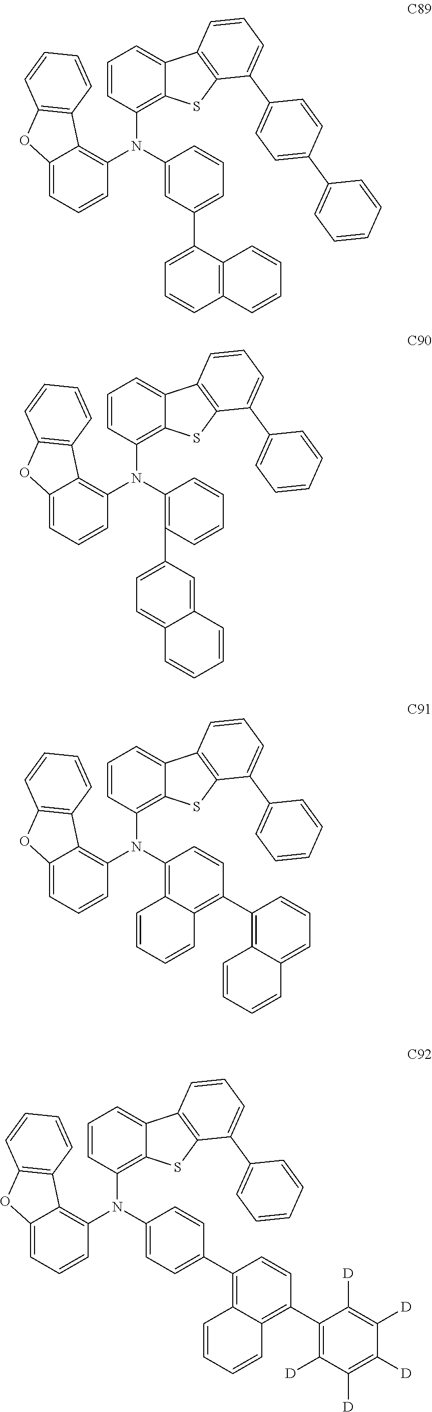

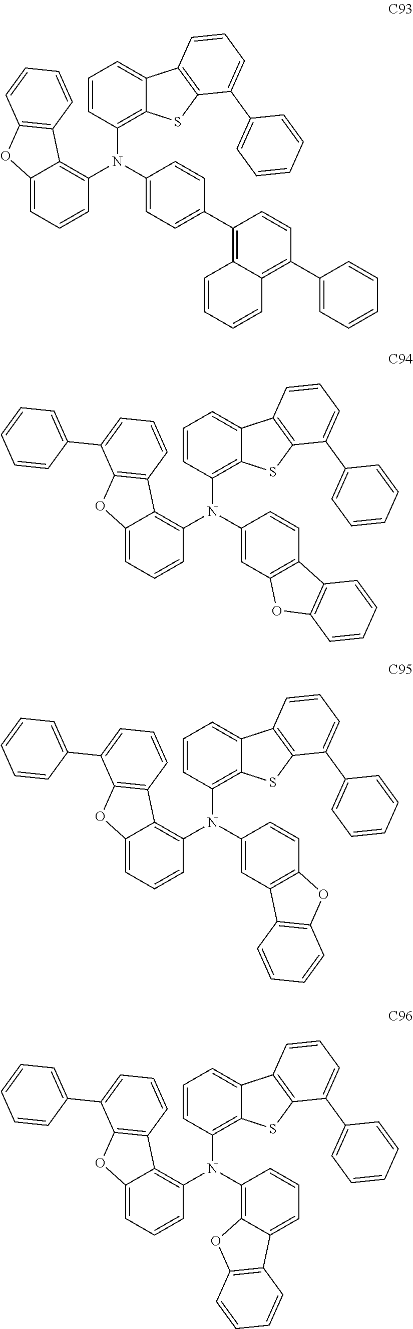 [SMILES: c1ccc(-c2ccc(-c3ccc(N(c4cccc5c4sc4c(-c6ccccc6)cccc45)c4cccc5oc6ccccc6c45)cc3)c3ccccc23)cc1 ; c1ccc(-c2cccc3c2oc2cccc(N(c4ccc5c(c4)oc4ccccc45)c4cccc5c4sc4c(-c6ccccc6)cccc45)c23)cc1 ; c1ccc(-c2cccc3c2oc2cccc(N(c4ccc5oc6ccccc6c5c4)c4cccc5c4sc4c(-c6ccccc6)cccc45)c23)cc1 ; c1ccc(-c2cccc3c2oc2cccc(N(c4cccc5c4oc4ccccc45)c4cccc5c4sc4c(-c6ccccc6)cccc45)c23)cc1]

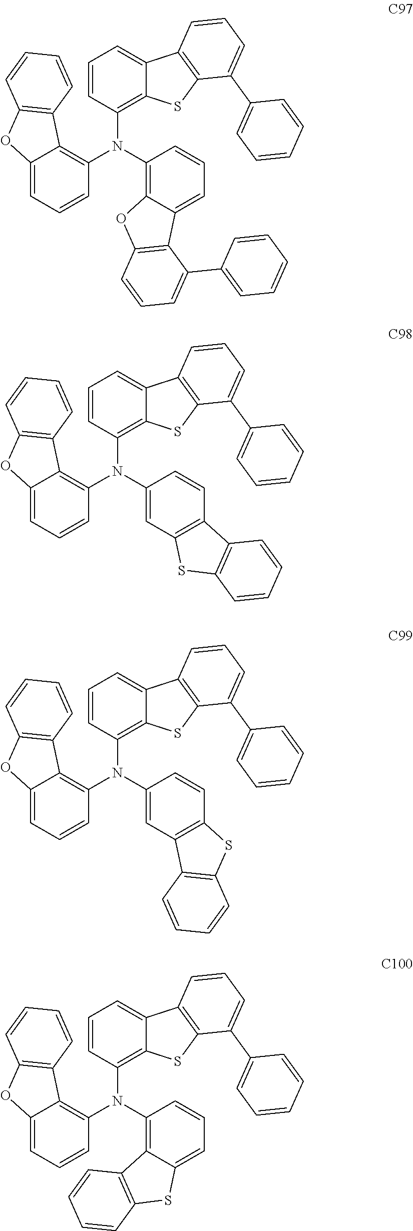

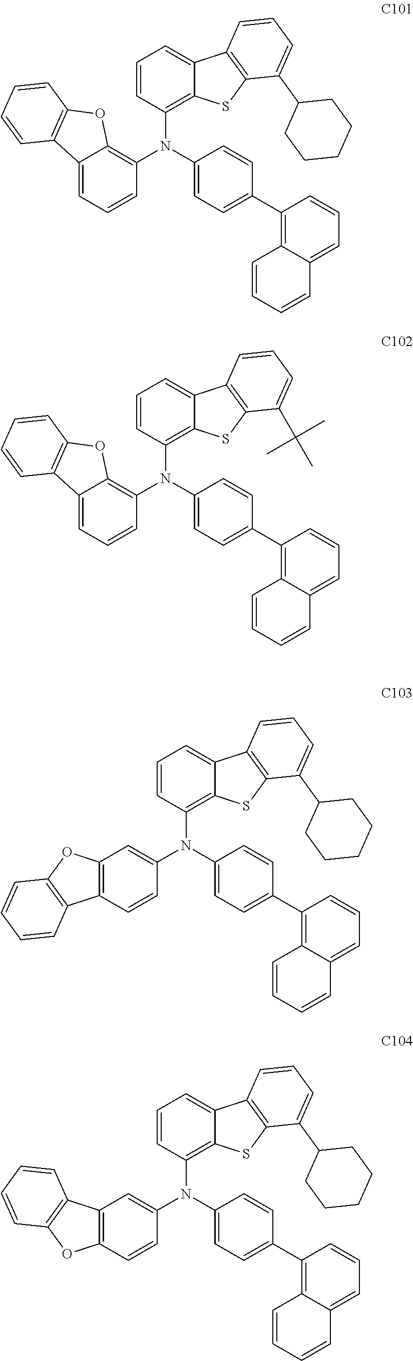

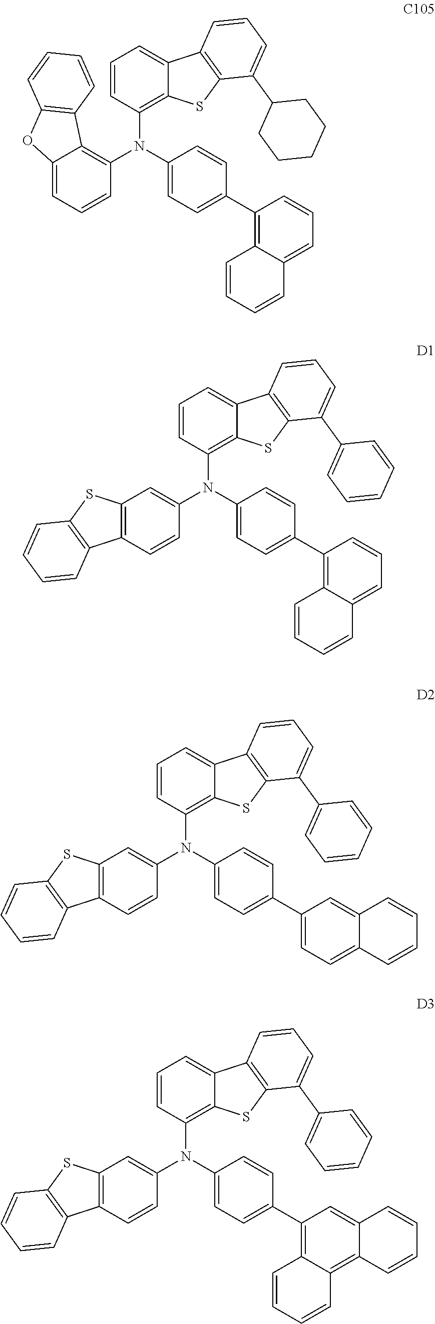 [SMILES: c1ccc2c(-c3ccc(N(c4cccc5c4sc4c(C6CCCCC6)cccc45)c4cccc5oc6ccccc6c45)cc3)cccc2c1 ; c1ccc(-c2cccc3c2sc2c(N(c4ccc(-c5cccc6ccccc56)cc4)c4ccc5c(c4)sc4ccccc45)cccc23)cc1 ; c1ccc(-c2cccc3c2sc2c(N(c4ccc(-c5ccc6ccccc6c5)cc4)c4ccc5c(c4)sc4ccccc45)cccc23)cc1 ; c1ccc(-c2cccc3c2sc2c(N(c4ccc(-c5cc6ccccc6c6ccccc56)cc4)c4ccc5c(c4)sc4ccccc45)cccc23)cc1]

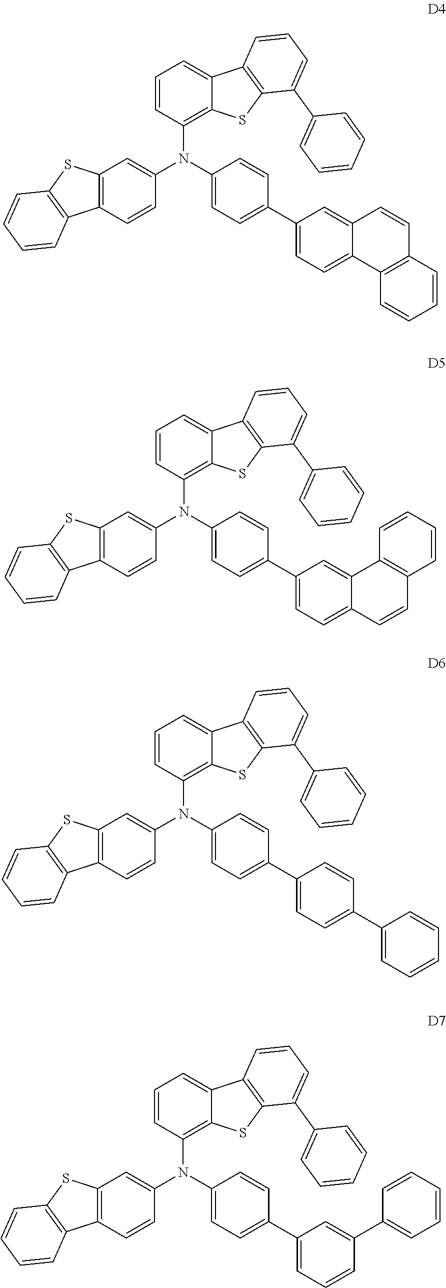 [SMILES: c1ccc(-c2cccc3c2sc2c(N(c4ccc(-c5ccc6c(ccc7ccccc76)c5)cc4)c4ccc5c(c4)sc4ccccc45)cccc23)cc1 ; c1ccc(-c2cccc3c2sc2c(N(c4ccc(-c5ccc6ccc7ccccc7c6c5)cc4)c4ccc5c(c4)sc4ccccc45)cccc23)cc1 ; c1ccc(-c2ccc(-c3ccc(N(c4ccc5c(c4)sc4ccccc45)c4cccc5c4sc4c(-c6ccccc6)cccc45)cc3)cc2)cc1 ; c1ccc(-c2cccc(-c3ccc(N(c4ccc5c(c4)sc4ccccc45)c4cccc5c4sc4c(-c6ccccc6)cccc45)cc3)c2)cc1]

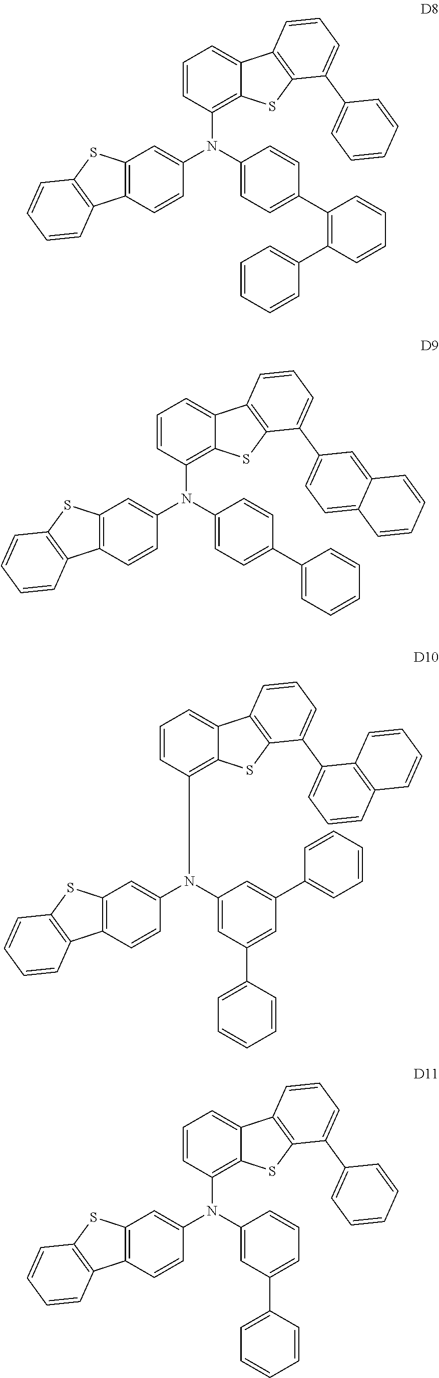 [SMILES: c1ccc(-c2ccccc2-c2ccc(N(c3ccc4c(c3)sc3ccccc34)c3cccc4c3sc3c(-c5ccccc5)cccc34)cc2)cc1 ; c1ccc(-c2ccc(N(c3ccc4c(c3)sc3ccccc34)c3cccc4c3sc3c(-c5ccc6ccccc6c5)cccc34)cc2)cc1 ; c1ccc(-c2cc(-c3ccccc3)cc(N(c3ccc4c(c3)sc3ccccc34)c3cccc4c3sc3c(-c5cccc6ccccc56)cccc34)c2)cc1 ; c1ccc(-c2cccc(N(c3ccc4c(c3)sc3ccccc34)c3cccc4c3sc3c(-c5ccccc5)cccc34)c2)cc1]

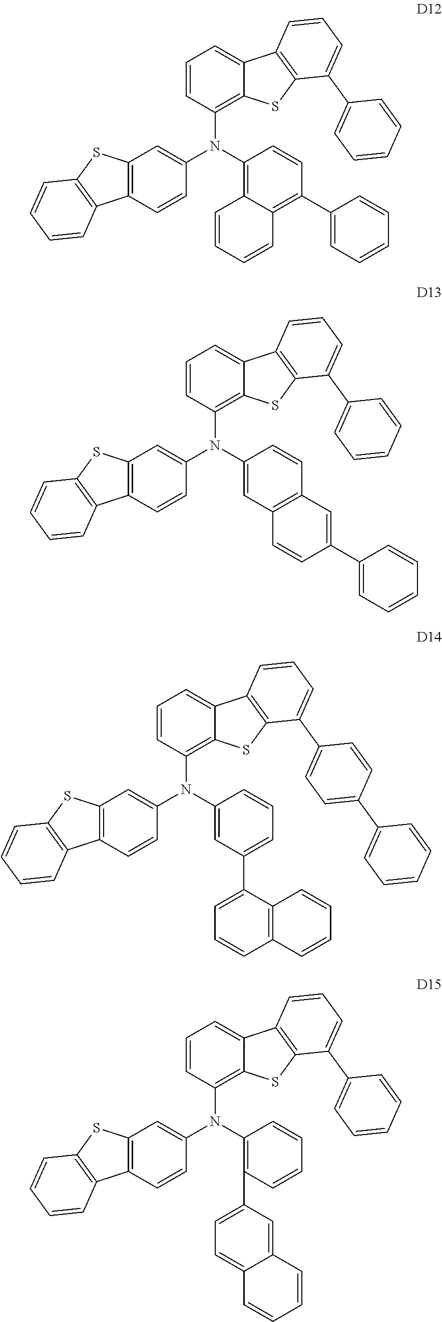

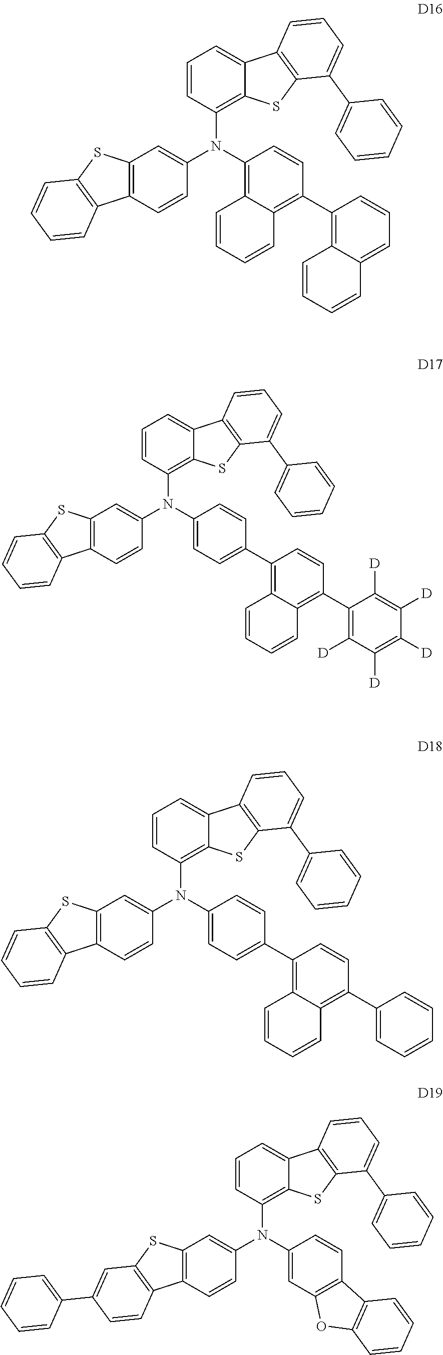 [SMILES: c1ccc(-c2cccc3c2sc2c(N(c4ccc5c(c4)sc4ccccc45)c4ccc(-c5cccc6ccccc56)c5ccccc45)cccc23)cc1 ; [2H]c1c([2H])c([2H])c(-c2ccc(-c3ccc(N(c4ccc5c(c4)sc4ccccc45)c4cccc5c4sc4c(-c6ccccc6)cccc45)cc3)c3ccccc23)c([2H])c1[2H] ; c1ccc(-c2ccc(-c3ccc(N(c4ccc5c(c4)sc4ccccc45)c4cccc5c4sc4c(-c6ccccc6)cccc45)cc3)c3ccccc23)cc1 ; c1ccc(-c2ccc3c(c2)sc2cc(N(c4ccc5c(c4)oc4ccccc45)c4cccc5c4sc4c(-c6ccccc6)cccc45)ccc23)cc1]

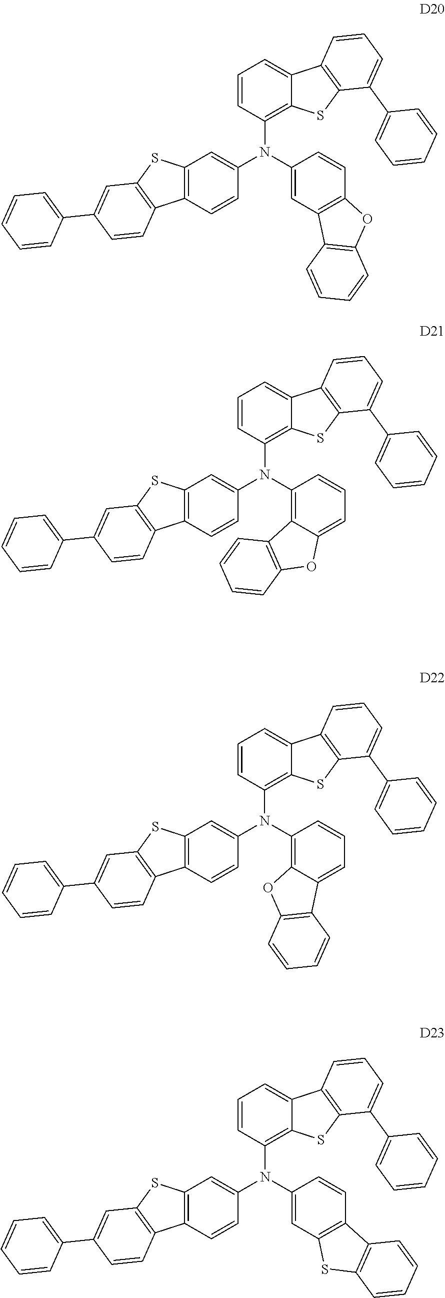 [SMILES: c1ccc(-c2ccc3c(c2)sc2cc(N(c4ccc5oc6ccccc6c5c4)c4cccc5c4sc4c(-c6ccccc6)cccc45)ccc23)cc1 ; c1ccc(-c2ccc3c(c2)sc2cc(N(c4cccc5c4sc4c(-c6ccccc6)cccc45)c4cccc5oc6ccccc6c45)ccc23)cc1 ; c1ccc(-c2ccc3c(c2)sc2cc(N(c4cccc5c4oc4ccccc45)c4cccc5c4sc4c(-c6ccccc6)cccc45)ccc23)cc1 ; c1ccc(-c2ccc3c(c2)sc2cc(N(c4ccc5c(c4)sc4ccccc45)c4cccc5c4sc4c(-c6ccccc6)cccc45)ccc23)cc1]

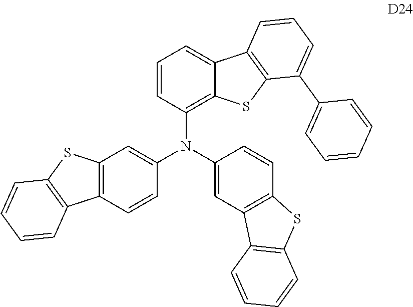 [SMILES: c1ccc(-c2cccc3c2sc2c(N(c4ccc5c(c4)sc4ccccc45)c4ccc5sc6ccccc6c5c4)cccc23)cc1]

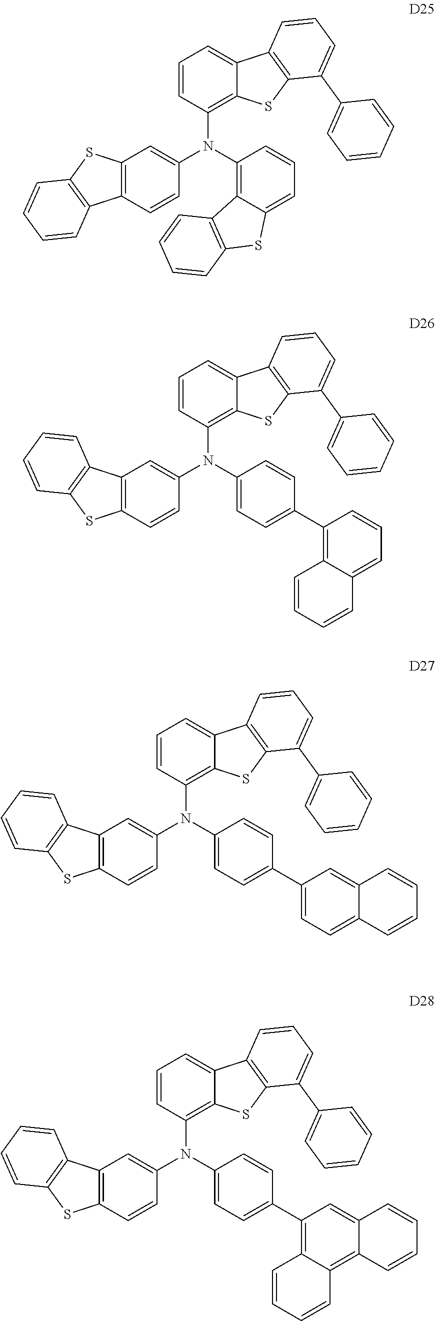 [SMILES: c1ccc(-c2cccc3c2sc2c(N(c4ccc5c(c4)sc4ccccc45)c4cccc5sc6ccccc6c45)cccc23)cc1 ; c1ccc(-c2cccc3c2sc2c(N(c4ccc(-c5cccc6ccccc56)cc4)c4ccc5sc6ccccc6c5c4)cccc23)cc1 ; c1ccc(-c2cccc3c2sc2c(N(c4ccc(-c5ccc6ccccc6c5)cc4)c4ccc5sc6ccccc6c5c4)cccc23)cc1 ; c1ccc(-c2cccc3c2sc2c(N(c4ccc(-c5cc6ccccc6c6ccccc56)cc4)c4ccc5sc6ccccc6c5c4)cccc23)cc1]

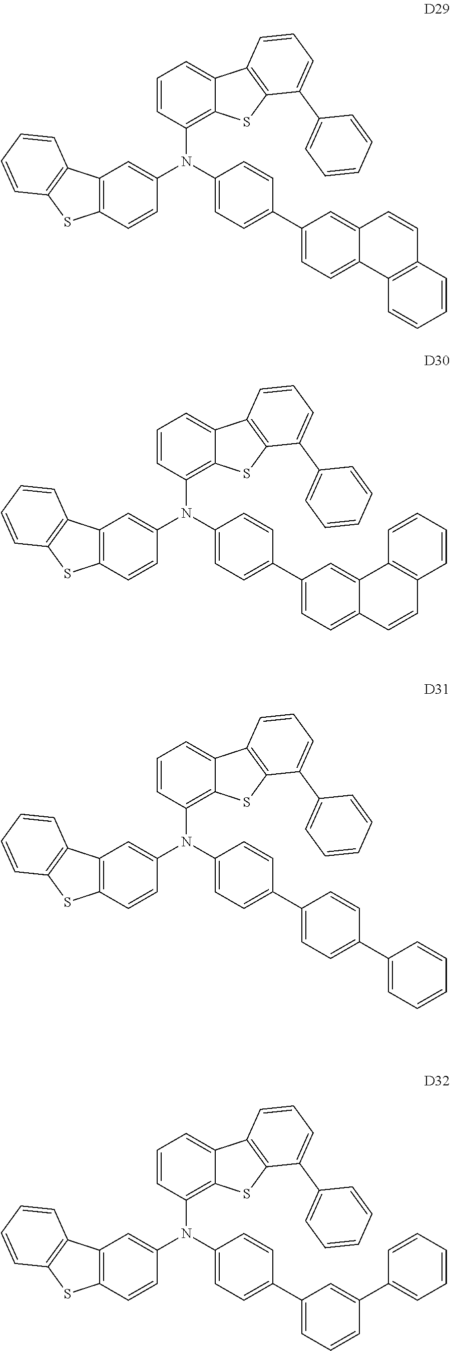 [SMILES: c1ccc(-c2cccc3c2sc2c(N(c4ccc(-c5ccc6c(ccc7ccccc76)c5)cc4)c4ccc5sc6ccccc6c5c4)cccc23)cc1 ; c1ccc(-c2cccc3c2sc2c(N(c4ccc(-c5ccc6ccc7ccccc7c6c5)cc4)c4ccc5sc6ccccc6c5c4)cccc23)cc1 ; c1ccc(-c2ccc(-c3ccc(N(c4ccc5sc6ccccc6c5c4)c4cccc5c4sc4c(-c6ccccc6)cccc45)cc3)cc2)cc1 ; c1ccc(-c2cccc(-c3ccc(N(c4ccc5sc6ccccc6c5c4)c4cccc5c4sc4c(-c6ccccc6)cccc45)cc3)c2)cc1]

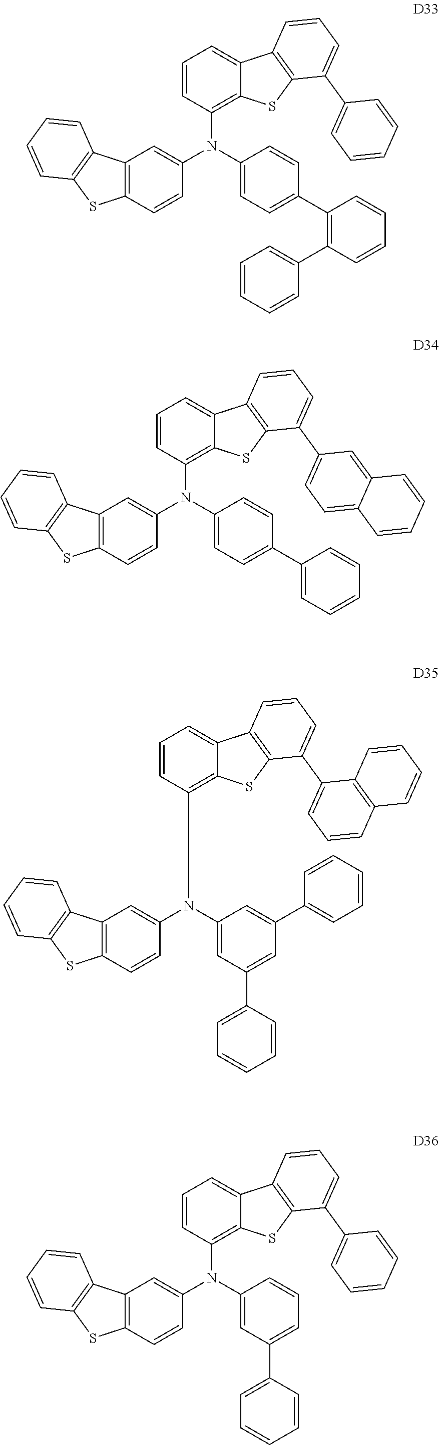 [SMILES: c1ccc(-c2ccccc2-c2ccc(N(c3ccc4sc5ccccc5c4c3)c3cccc4c3sc3c(-c5ccccc5)cccc34)cc2)cc1 ; c1ccc(-c2ccc(N(c3ccc4sc5ccccc5c4c3)c3cccc4c3sc3c(-c5ccc6ccccc6c5)cccc34)cc2)cc1 ; c1ccc(-c2cc(-c3ccccc3)cc(N(c3ccc4sc5ccccc5c4c3)c3cccc4c3sc3c(-c5cccc6ccccc56)cccc34)c2)cc1 ; c1ccc(-c2cccc(N(c3ccc4sc5ccccc5c4c3)c3cccc4c3sc3c(-c5ccccc5)cccc34)c2)cc1]

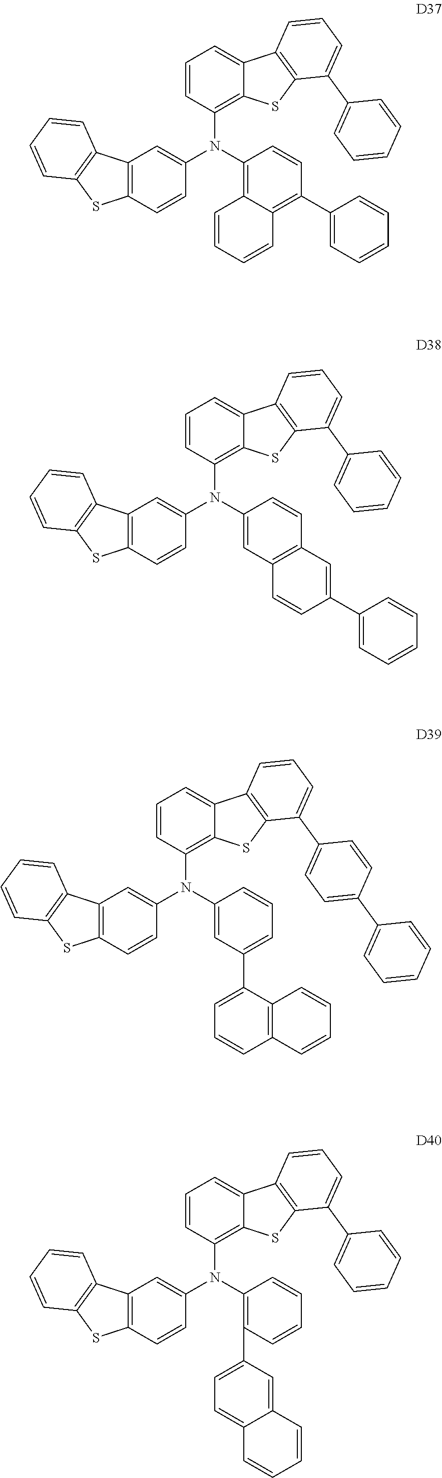 [SMILES: c1ccc(-c2ccc(N(c3ccc4sc5ccccc5c4c3)c3cccc4c3sc3c(-c5ccccc5)cccc34)c3ccccc23)cc1 ; c1ccc(-c2ccc3cc(N(c4ccc5sc6ccccc6c5c4)c4cccc5c4sc4c(-c6ccccc6)cccc45)ccc3c2)cc1 ; c1ccc(-c2ccc(-c3cccc4c3sc3c(N(c5cccc(-c6cccc7ccccc67)c5)c5ccc6sc7ccccc7c6c5)cccc34)cc2)cc1 ; c1ccc(-c2cccc3c2sc2c(N(c4ccc5sc6ccccc6c5c4)c4ccccc4-c4ccc5ccccc5c4)cccc23)cc1]

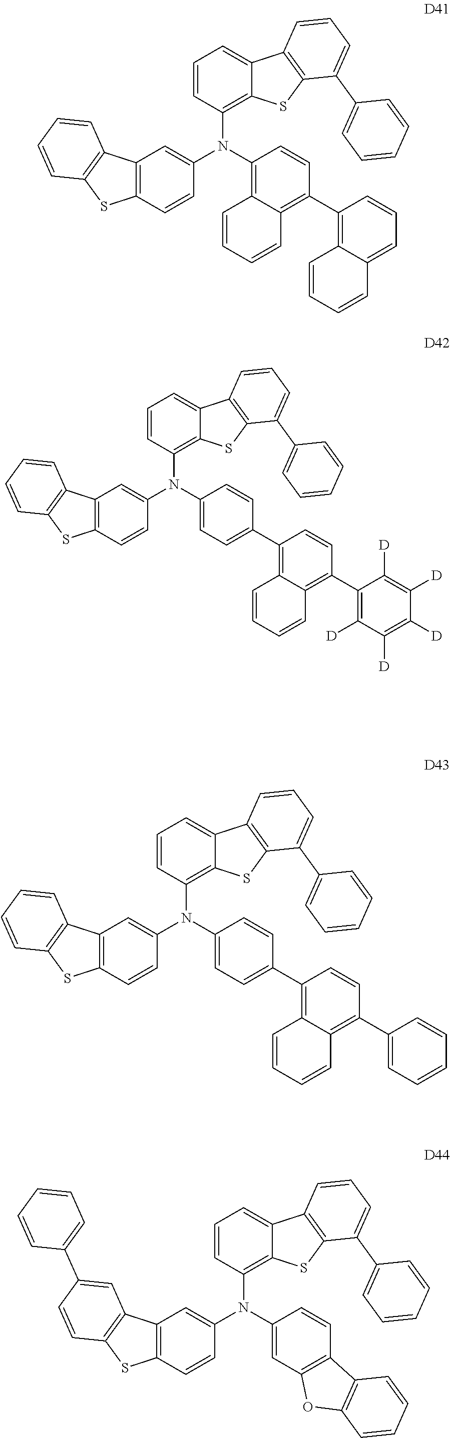 [SMILES: c1ccc(-c2cccc3c2sc2c(N(c4ccc5sc6ccccc6c5c4)c4ccc(-c5cccc6ccccc56)c5ccccc45)cccc23)cc1 ; [2H]c1c([2H])c([2H])c(-c2ccc(-c3ccc(N(c4ccc5sc6ccccc6c5c4)c4cccc5c4sc4c(-c6ccccc6)cccc45)cc3)c3ccccc23)c([2H])c1[2H] ; c1ccc(-c2ccc(-c3ccc(N(c4ccc5sc6ccccc6c5c4)c4cccc5c4sc4c(-c6ccccc6)cccc45)cc3)c3ccccc23)cc1 ; c1ccc(-c2ccc3sc4ccc(N(c5ccc6c(c5)oc5ccccc56)c5cccc6c5sc5c(-c7ccccc7)cccc56)cc4c3c2)cc1]

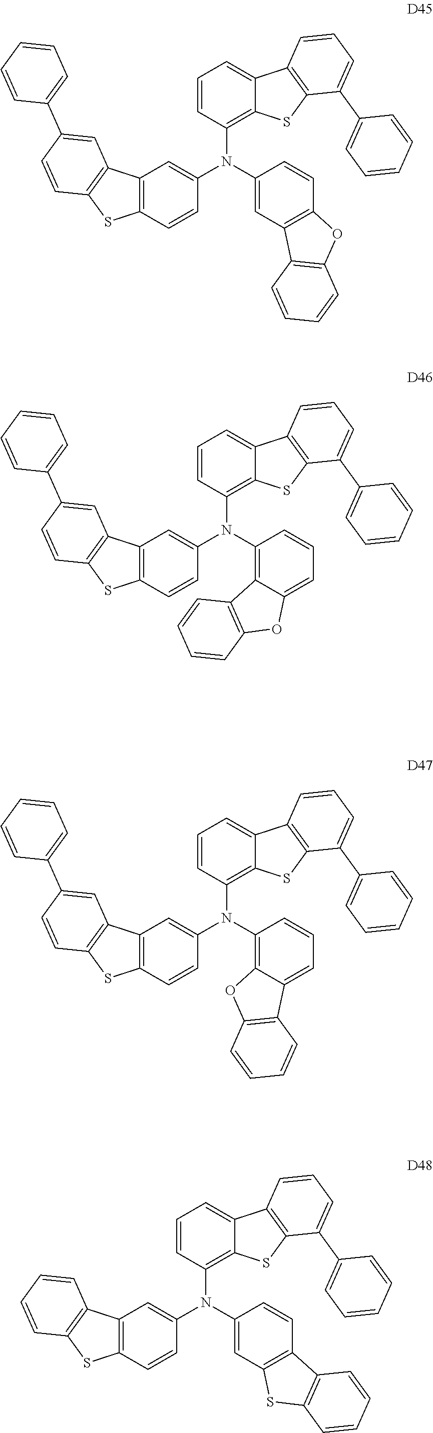 [SMILES: c1ccc(-c2ccc3sc4ccc(N(c5ccc6oc7ccccc7c6c5)c5cccc6c5sc5c(-c7ccccc7)cccc56)cc4c3c2)cc1 ; c1ccc(-c2ccc3sc4ccc(N(c5cccc6c5sc5c(-c7ccccc7)cccc56)c5cccc6oc7ccccc7c56)cc4c3c2)cc1 ; c1ccc(-c2ccc3sc4ccc(N(c5cccc6c5oc5ccccc56)c5cccc6c5sc5c(-c7ccccc7)cccc56)cc4c3c2)cc1 ; c1ccc(-c2cccc3c2sc2c(N(c4ccc5c(c4)sc4ccccc45)c4ccc5sc6ccccc6c5c4)cccc23)cc1]

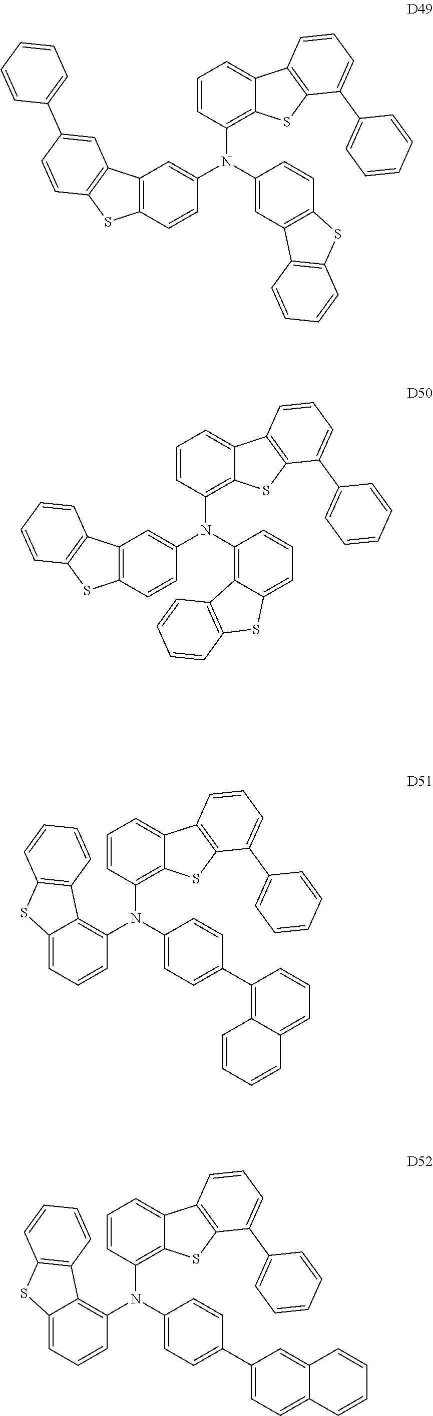 [SMILES: c1ccc(-c2ccc3sc4ccc(N(c5ccc6sc7ccccc7c6c5)c5cccc6c5sc5c(-c7ccccc7)cccc56)cc4c3c2)cc1 ; c1ccc(-c2cccc3c2sc2c(N(c4ccc5sc6ccccc6c5c4)c4cccc5sc6ccccc6c45)cccc23)cc1 ; c1ccc(-c2cccc3c2sc2c(N(c4ccc(-c5cccc6ccccc56)cc4)c4cccc5sc6ccccc6c45)cccc23)cc1 ; c1ccc(-c2cccc3c2sc2c(N(c4ccc(-c5ccc6ccccc6c5)cc4)c4cccc5sc6ccccc6c45)cccc23)cc1]

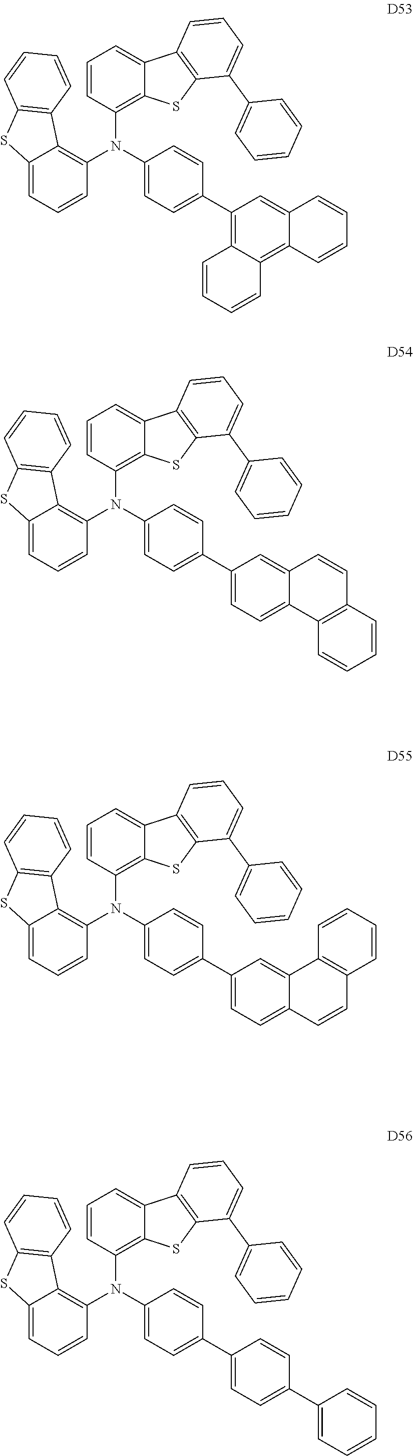 [SMILES: c1ccc(-c2cccc3c2sc2c(N(c4ccc(-c5cc6ccccc6c6ccccc56)cc4)c4cccc5sc6ccccc6c45)cccc23)cc1 ; c1ccc(-c2cccc3c2sc2c(N(c4ccc(-c5ccc6c(ccc7ccccc76)c5)cc4)c4cccc5sc6ccccc6c45)cccc23)cc1 ; c1ccc(-c2cccc3c2sc2c(N(c4ccc(-c5ccc6ccc7ccccc7c6c5)cc4)c4cccc5sc6ccccc6c45)cccc23)cc1 ; c1ccc(-c2ccc(-c3ccc(N(c4cccc5c4sc4c(-c6ccccc6)cccc45)c4cccc5sc6ccccc6c45)cc3)cc2)cc1]

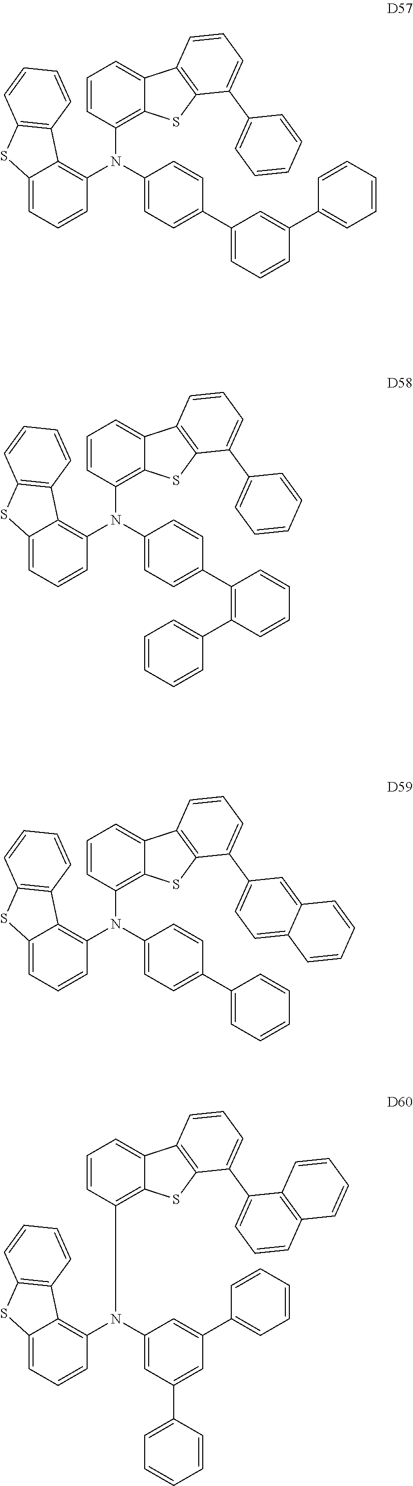 [SMILES: c1ccc(-c2cccc(-c3ccc(N(c4cccc5c4sc4c(-c6ccccc6)cccc45)c4cccc5sc6ccccc6c45)cc3)c2)cc1 ; c1ccc(-c2ccccc2-c2ccc(N(c3cccc4c3sc3c(-c5ccccc5)cccc34)c3cccc4sc5ccccc5c34)cc2)cc1 ; c1ccc(-c2ccc(N(c3cccc4c3sc3c(-c5ccc6ccccc6c5)cccc34)c3cccc4sc5ccccc5c34)cc2)cc1 ; c1ccc(-c2cc(-c3ccccc3)cc(N(c3cccc4c3sc3c(-c5cccc6ccccc56)cccc34)c3cccc4sc5ccccc5c34)c2)cc1]

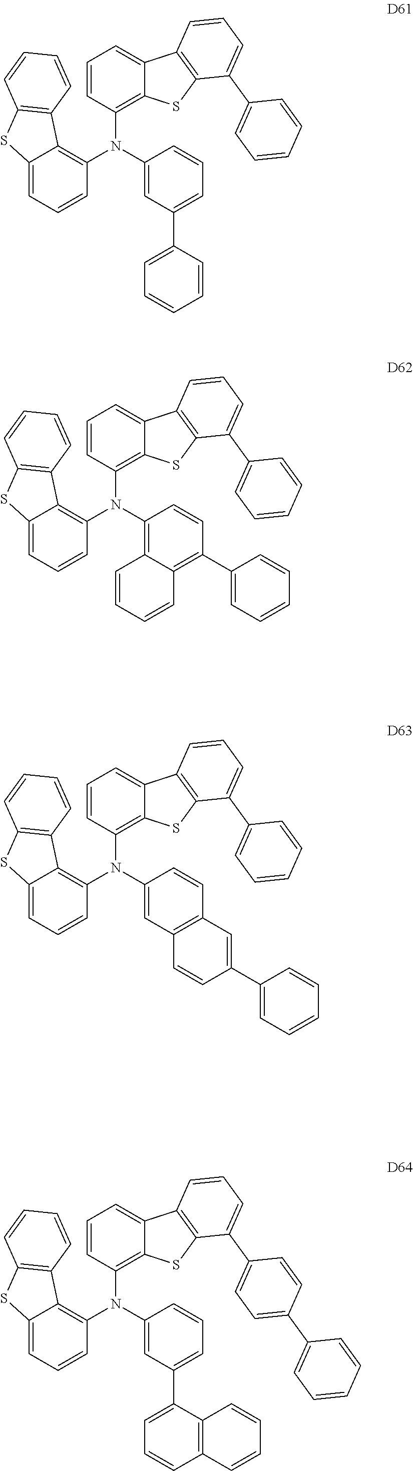 [SMILES: c1ccc(-c2cccc(N(c3cccc4c3sc3c(-c5ccccc5)cccc34)c3cccc4sc5ccccc5c34)c2)cc1 ; c1ccc(-c2ccc(N(c3cccc4c3sc3c(-c5ccccc5)cccc34)c3cccc4sc5ccccc5c34)c3ccccc23)cc1 ; c1ccc(-c2ccc3cc(N(c4cccc5c4sc4c(-c6ccccc6)cccc45)c4cccc5sc6ccccc6c45)ccc3c2)cc1 ; c1ccc(-c2ccc(-c3cccc4c3sc3c(N(c5cccc(-c6cccc7ccccc67)c5)c5cccc6sc7ccccc7c56)cccc34)cc2)cc1]

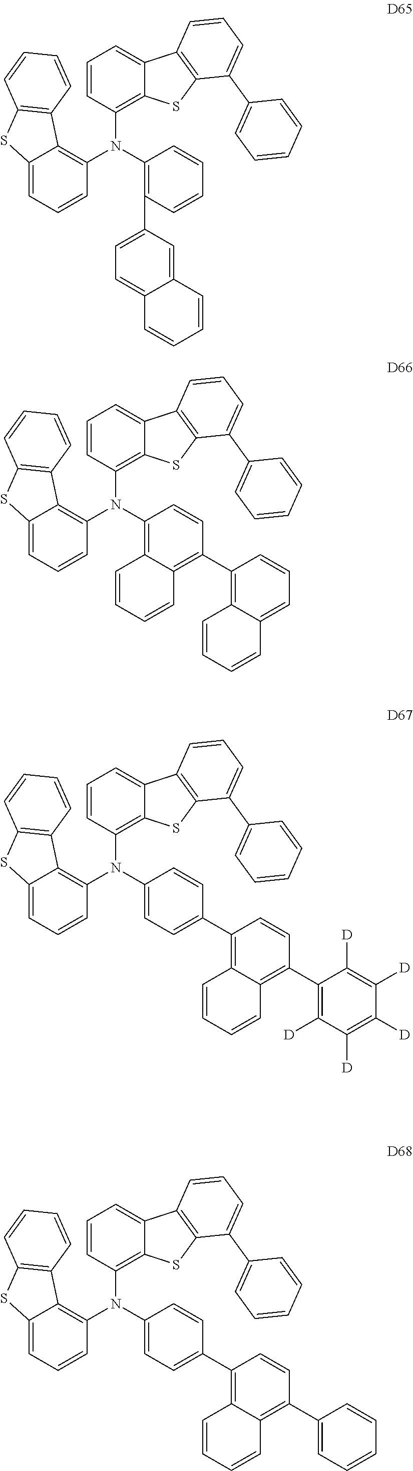

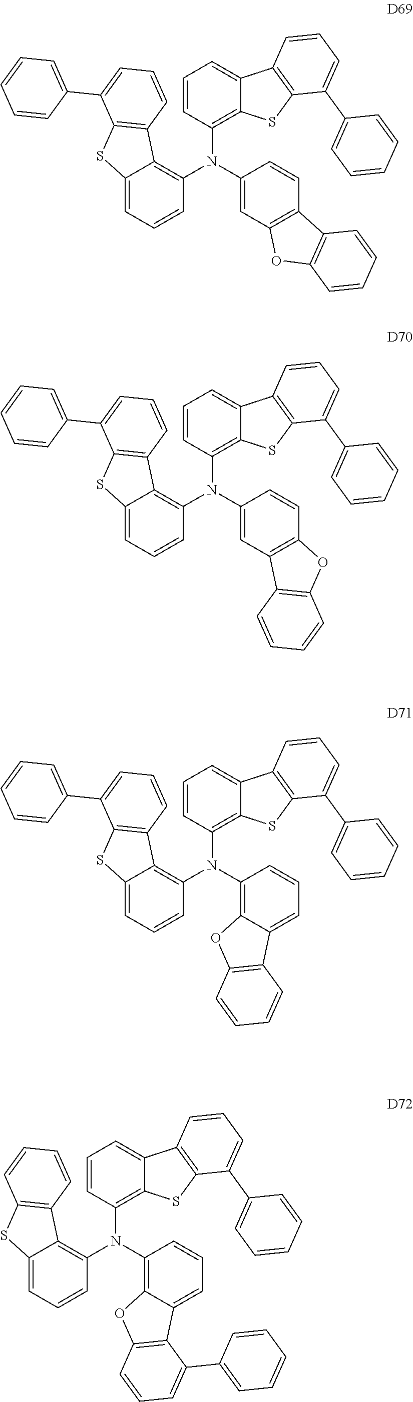 [SMILES: c1ccc(-c2cccc3c2sc2c(N(c4ccc5c(c4)oc4ccccc45)c4cccc5sc6c(-c7ccccc7)cccc6c45)cccc23)cc1 ; c1ccc(-c2cccc3c2sc2c(N(c4ccc5oc6ccccc6c5c4)c4cccc5sc6c(-c7ccccc7)cccc6c45)cccc23)cc1 ; c1ccc(-c2cccc3c2sc2c(N(c4cccc5c4oc4ccccc45)c4cccc5sc6c(-c7ccccc7)cccc6c45)cccc23)cc1 ; c1ccc(-c2cccc3c2sc2c(N(c4cccc5c4oc4cccc(-c6ccccc6)c45)c4cccc5sc6ccccc6c45)cccc23)cc1]

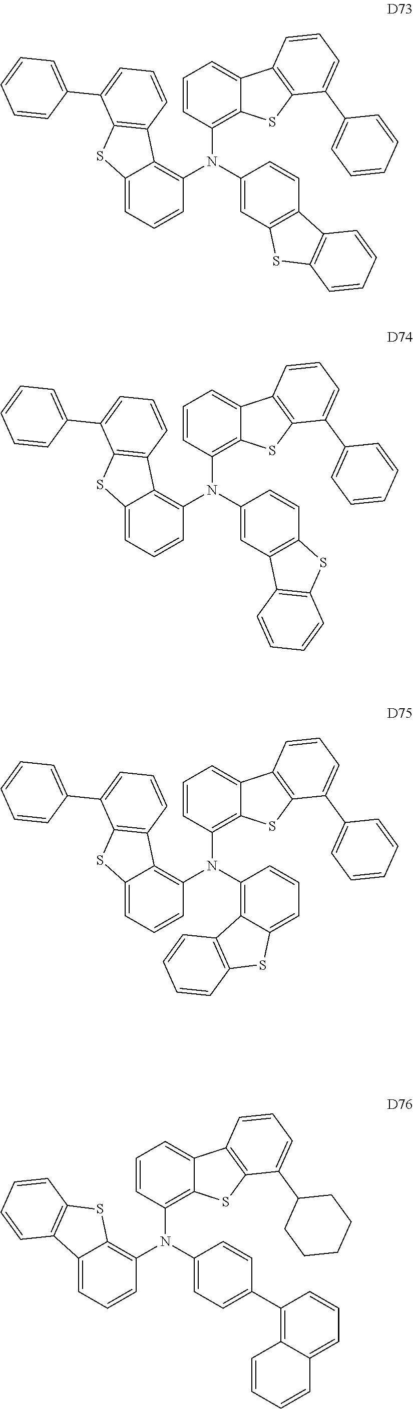 [SMILES: c1ccc(-c2cccc3c2sc2c(N(c4ccc5c(c4)sc4ccccc45)c4cccc5sc6c(-c7ccccc7)cccc6c45)cccc23)cc1 ; c1ccc(-c2cccc3c2sc2c(N(c4ccc5sc6ccccc6c5c4)c4cccc5sc6c(-c7ccccc7)cccc6c45)cccc23)cc1 ; c1ccc(-c2cccc3c2sc2c(N(c4cccc5sc6ccccc6c45)c4cccc5sc6c(-c7ccccc7)cccc6c45)cccc23)cc1 ; c1ccc2c(-c3ccc(N(c4cccc5c4sc4ccccc45)c4cccc5c4sc4c(C6CCCCC6)cccc45)cc3)cccc2c1]

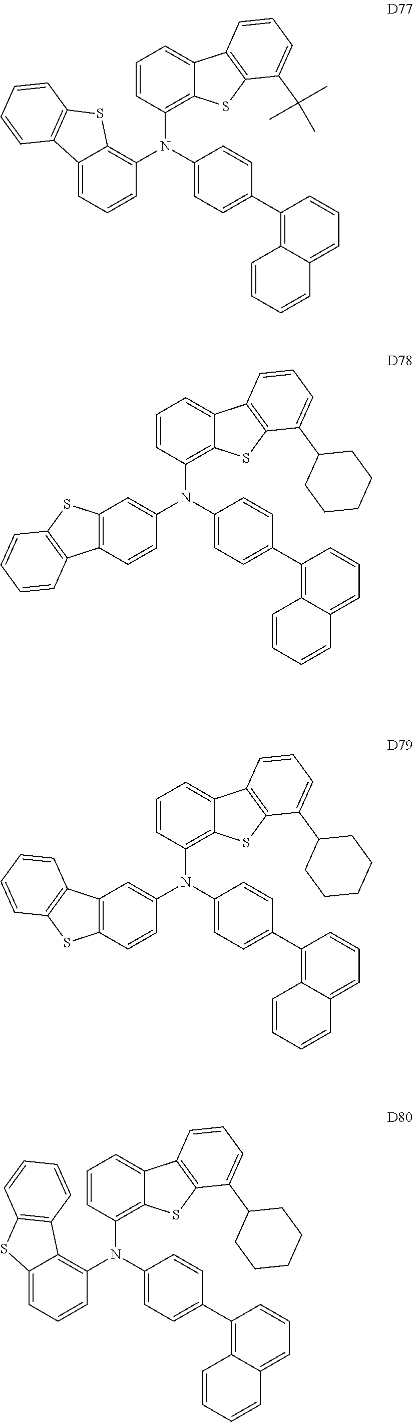 [SMILES: CC(C)(C)c1cccc2c1sc1c(N(c3ccc(-c4cccc5ccccc45)cc3)c3cccc4c3sc3ccccc34)cccc12 ; c1ccc2c(-c3ccc(N(c4ccc5c(c4)sc4ccccc45)c4cccc5c4sc4c(C6CCCCC6)cccc45)cc3)cccc2c1 ; c1ccc2c(-c3ccc(N(c4ccc5sc6ccccc6c5c4)c4cccc5c4sc4c(C6CCCCC6)cccc45)cc3)cccc2c1 ; c1ccc2c(-c3ccc(N(c4cccc5c4sc4c(C6CCCCC6)cccc45)c4cccc5sc6ccccc6c45)cc3)cccc2c1]

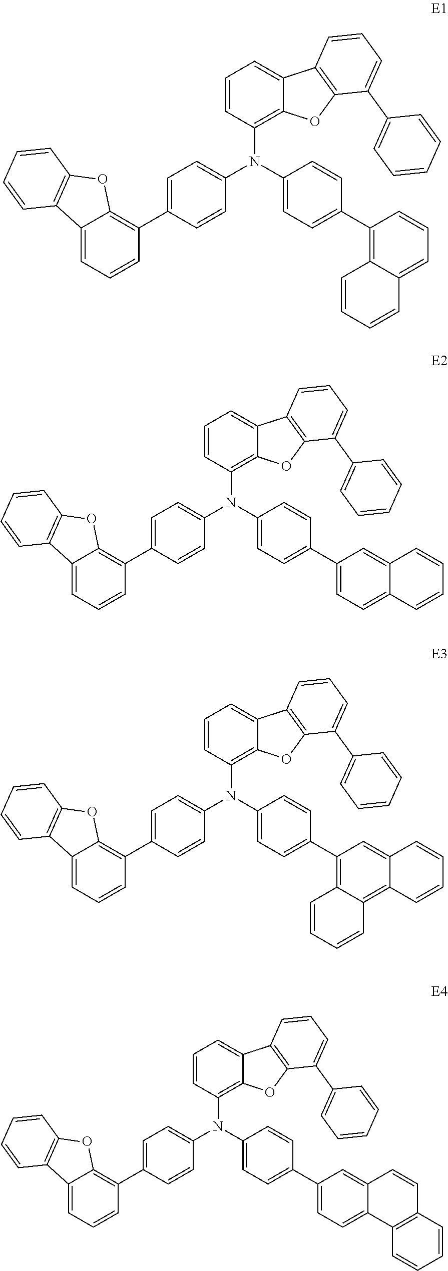 [SMILES: c1ccc(-c2cccc3c2oc2c(N(c4ccc(-c5cccc6ccccc56)cc4)c4ccc(-c5cccc6c5oc5ccccc56)cc4)cccc23)cc1 ; c1ccc(-c2cccc3c2oc2c(N(c4ccc(-c5ccc6ccccc6c5)cc4)c4ccc(-c5cccc6c5oc5ccccc56)cc4)cccc23)cc1 ; c1ccc(-c2cccc3c2oc2c(N(c4ccc(-c5cc6ccccc6c6ccccc56)cc4)c4ccc(-c5cccc6c5oc5ccccc56)cc4)cccc23)cc1 ; c1ccc(-c2cccc3c2oc2c(N(c4ccc(-c5ccc6c(ccc7ccccc76)c5)cc4)c4ccc(-c5cccc6c5oc5ccccc56)cc4)cccc23)cc1]

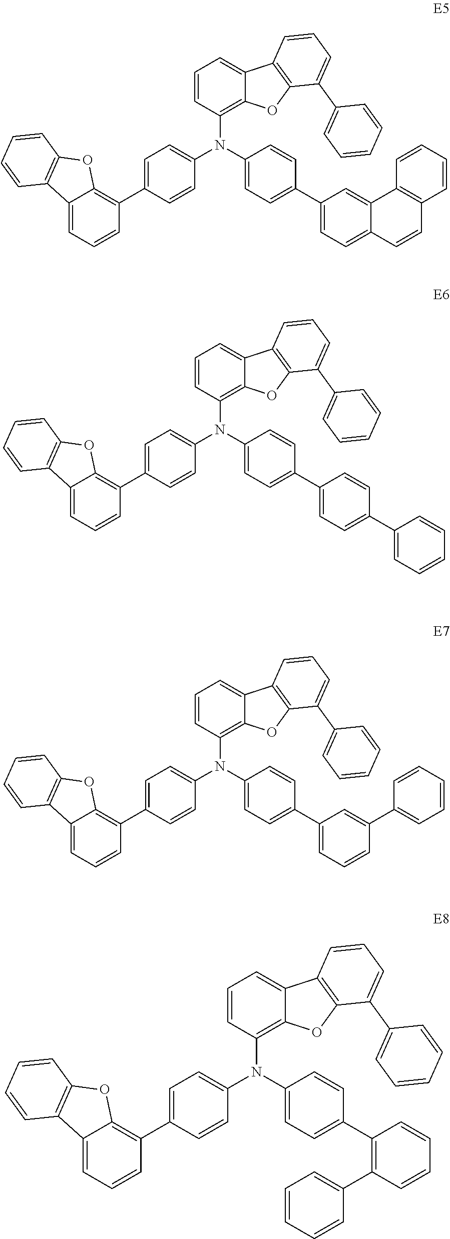 [SMILES: c1ccc(-c2cccc3c2oc2c(N(c4ccc(-c5ccc6ccc7ccccc7c6c5)cc4)c4ccc(-c5cccc6c5oc5ccccc56)cc4)cccc23)cc1 ; c1ccc(-c2ccc(-c3ccc(N(c4ccc(-c5cccc6c5oc5ccccc56)cc4)c4cccc5c4oc4c(-c6ccccc6)cccc45)cc3)cc2)cc1 ; c1ccc(-c2cccc(-c3ccc(N(c4ccc(-c5cccc6c5oc5ccccc56)cc4)c4cccc5c4oc4c(-c6ccccc6)cccc45)cc3)c2)cc1 ; c1ccc(-c2ccccc2-c2ccc(N(c3ccc(-c4cccc5c4oc4ccccc45)cc3)c3cccc4c3oc3c(-c5ccccc5)cccc34)cc2)cc1]

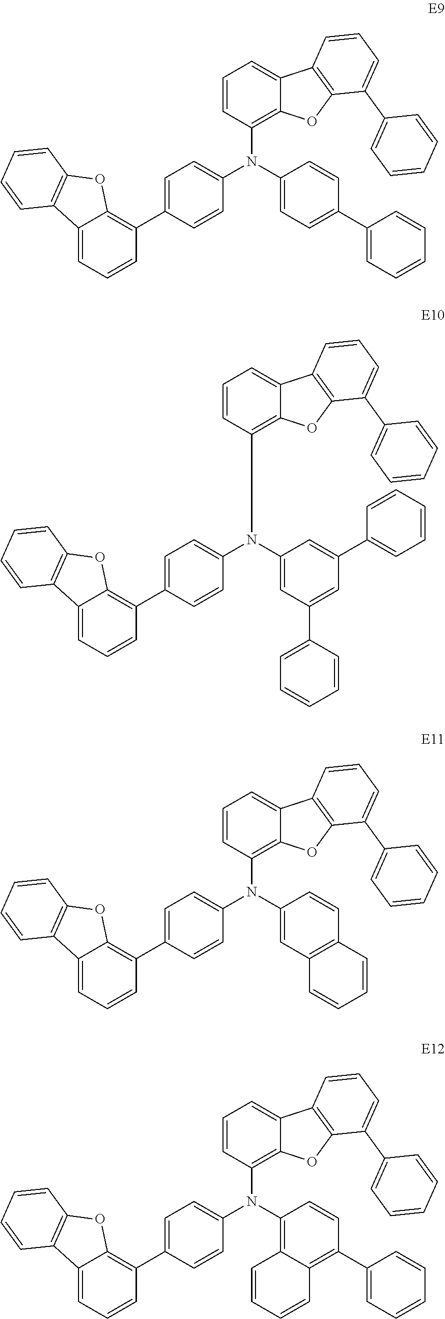 [SMILES: c1ccc(-c2ccc(N(c3ccc(-c4cccc5c4oc4ccccc45)cc3)c3cccc4c3oc3c(-c5ccccc5)cccc34)cc2)cc1 ; c1ccc(-c2cc(-c3ccccc3)cc(N(c3ccc(-c4cccc5c4oc4ccccc45)cc3)c3cccc4c3oc3c(-c5ccccc5)cccc34)c2)cc1 ; c1ccc(-c2cccc3c2oc2c(N(c4ccc(-c5cccc6c5oc5ccccc56)cc4)c4ccc5ccccc5c4)cccc23)cc1 ; c1ccc(-c2ccc(N(c3ccc(-c4cccc5c4oc4ccccc45)cc3)c3cccc4c3oc3c(-c5ccccc5)cccc34)c3ccccc23)cc1]

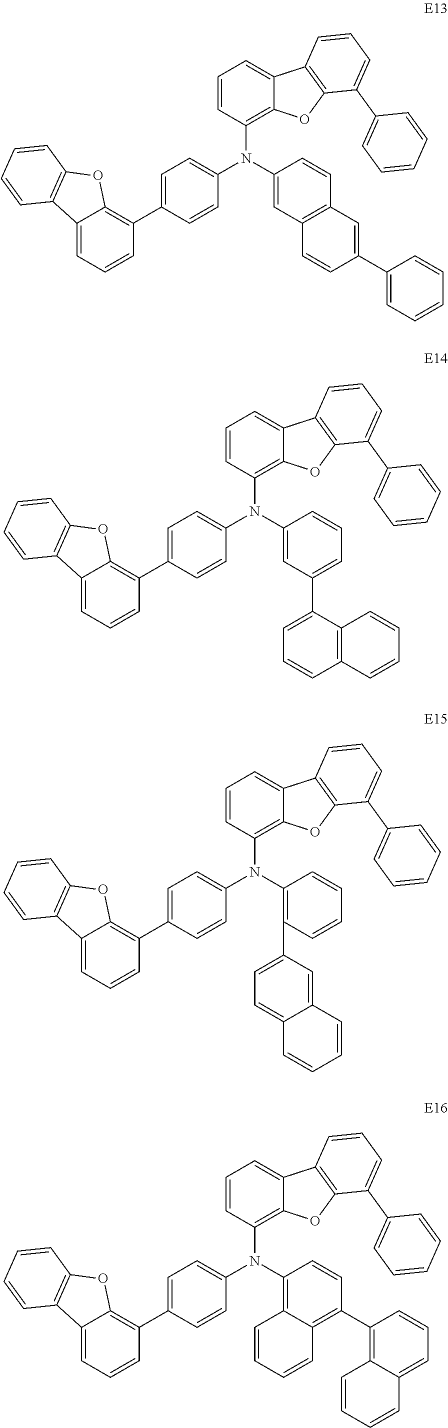 [SMILES: c1ccc(-c2ccc3cc(N(c4ccc(-c5cccc6c5oc5ccccc56)cc4)c4cccc5c4oc4c(-c6ccccc6)cccc45)ccc3c2)cc1 ; c1ccc(-c2cccc3c2oc2c(N(c4ccc(-c5cccc6c5oc5ccccc56)cc4)c4cccc(-c5cccc6ccccc56)c4)cccc23)cc1 ; c1ccc(-c2cccc3c2oc2c(N(c4ccc(-c5cccc6c5oc5ccccc56)cc4)c4ccccc4-c4ccc5ccccc5c4)cccc23)cc1 ; c1ccc(-c2cccc3c2oc2c(N(c4ccc(-c5cccc6c5oc5ccccc56)cc4)c4ccc(-c5cccc6ccccc56)c5ccccc45)cccc23)cc1]

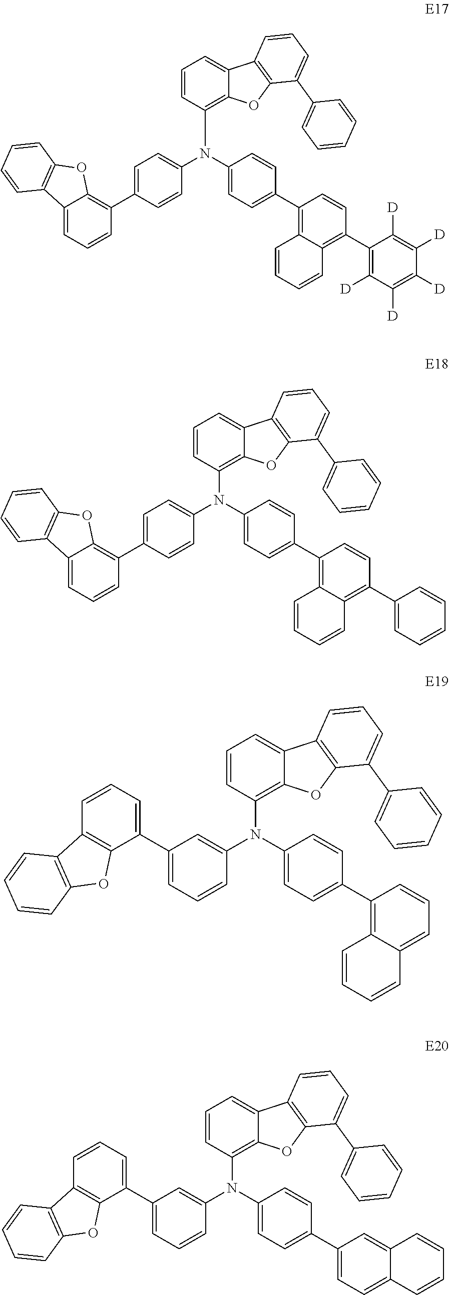 [SMILES: [2H]c1c([2H])c([2H])c(-c2ccc(-c3ccc(N(c4ccc(-c5cccc6c5oc5ccccc56)cc4)c4cccc5c4oc4c(-c6ccccc6)cccc45)cc3)c3ccccc23)c([2H])c1[2H] ; c1ccc(-c2ccc(-c3ccc(N(c4ccc(-c5cccc6c5oc5ccccc56)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5cccc6c5oc5ccccc56)c4)cccc23)cc1 ; c1ccc(-c2cccc3c2oc2c(N(c4ccc(-c5ccc6ccccc6c5)cc4)c4cccc(-c5cccc6c5oc5ccccc56)c4)cccc23)cc1]

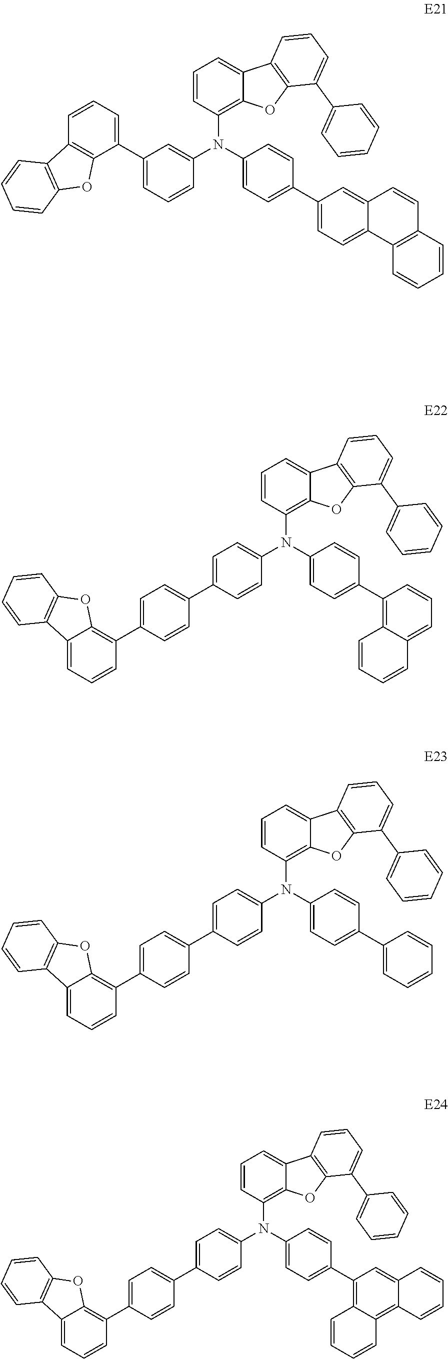 [SMILES: c1ccc(-c2cccc3c2oc2c(N(c4ccc(-c5ccc6c(ccc7ccccc76)c5)cc4)c4cccc(-c5cccc6c5oc5ccccc56)c4)cccc23)cc1 ; c1ccc(-c2cccc3c2oc2c(N(c4ccc(-c5ccc(-c6cccc7c6oc6ccccc67)cc5)cc4)c4ccc(-c5cccc6ccccc56)cc4)cccc23)cc1 ; c1ccc(-c2ccc(N(c3ccc(-c4ccc(-c5cccc6c5oc5ccccc56)cc4)cc3)c3cccc4c3oc3c(-c5ccccc5)cccc34)cc2)cc1 ; c1ccc(-c2cccc3c2oc2c(N(c4ccc(-c5ccc(-c6cccc7c6oc6ccccc67)cc5)cc4)c4ccc(-c5cc6ccccc6c6ccccc56)cc4)cccc23)cc1]

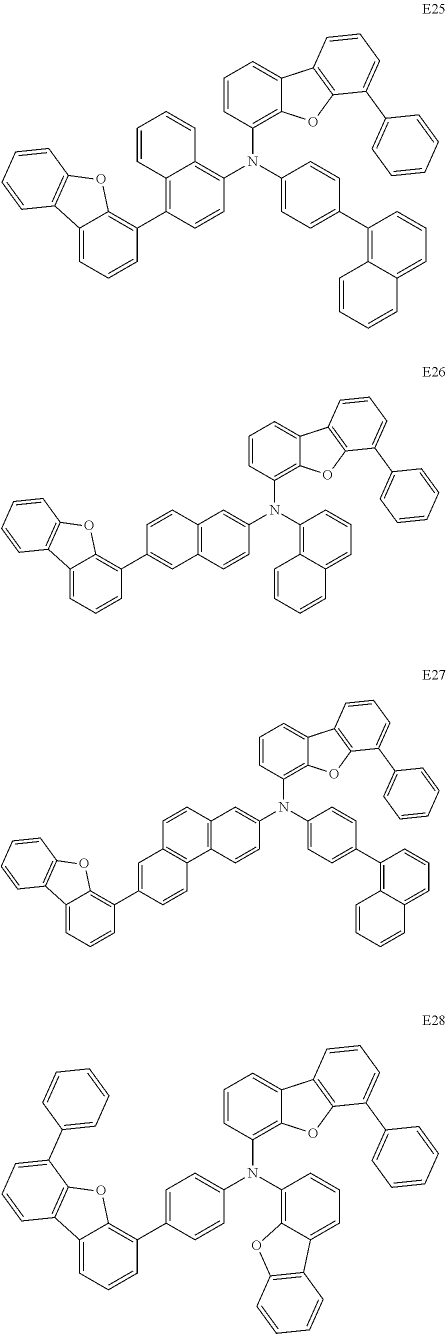 [SMILES: c1ccc(-c2cccc3c2oc2c(N(c4ccc(-c5cccc6ccccc56)cc4)c4ccc(-c5cccc6c5oc5ccccc56)c5ccccc45)cccc23)cc1 ; c1ccc(-c2cccc3c2oc2c(N(c4ccc5cc(-c6cccc7c6oc6ccccc67)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7cccc8c7oc7ccccc78)ccc65)c4)cccc23)cc1 ; c1ccc(-c2cccc3c2oc2c(-c4ccc(N(c5cccc6c5oc5ccccc56)c5cccc6c5oc5c(-c7ccccc7)cccc56)cc4)cccc23)cc1]

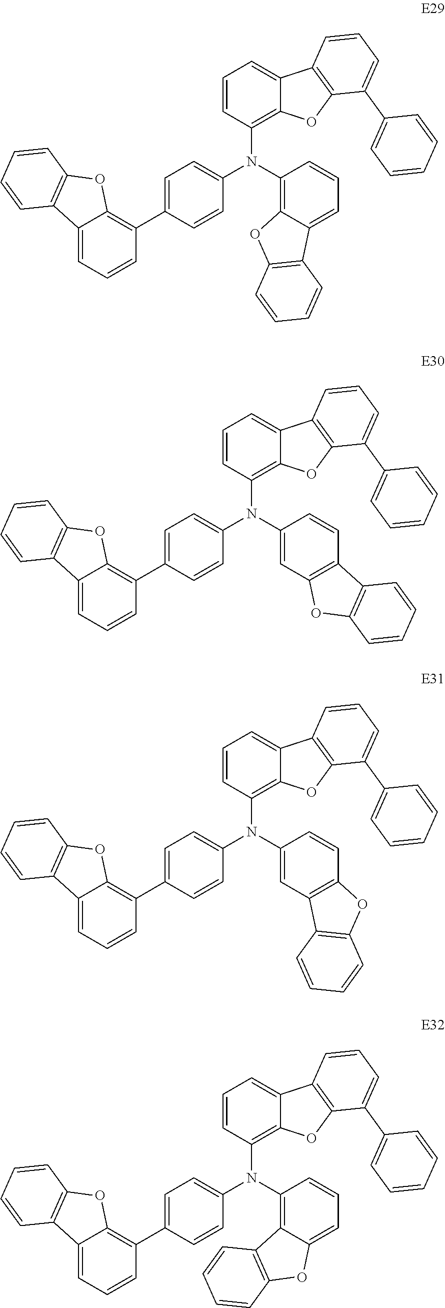 [SMILES: c1ccc(-c2cccc3c2oc2c(N(c4ccc(-c5cccc6c5oc5ccccc56)cc4)c4cccc5c4oc4ccccc45)cccc23)cc1 ; c1ccc(-c2cccc3c2oc2c(N(c4ccc(-c5cccc6c5oc5ccccc56)cc4)c4ccc5c(c4)oc4ccccc45)cccc23)cc1 ; c1ccc(-c2cccc3c2oc2c(N(c4ccc(-c5cccc6c5oc5ccccc56)cc4)c4ccc5oc6ccccc6c5c4)cccc23)cc1 ; c1ccc(-c2cccc3c2oc2c(N(c4ccc(-c5cccc6c5oc5ccccc56)cc4)c4cccc5oc6ccccc6c45)cccc23)cc1]

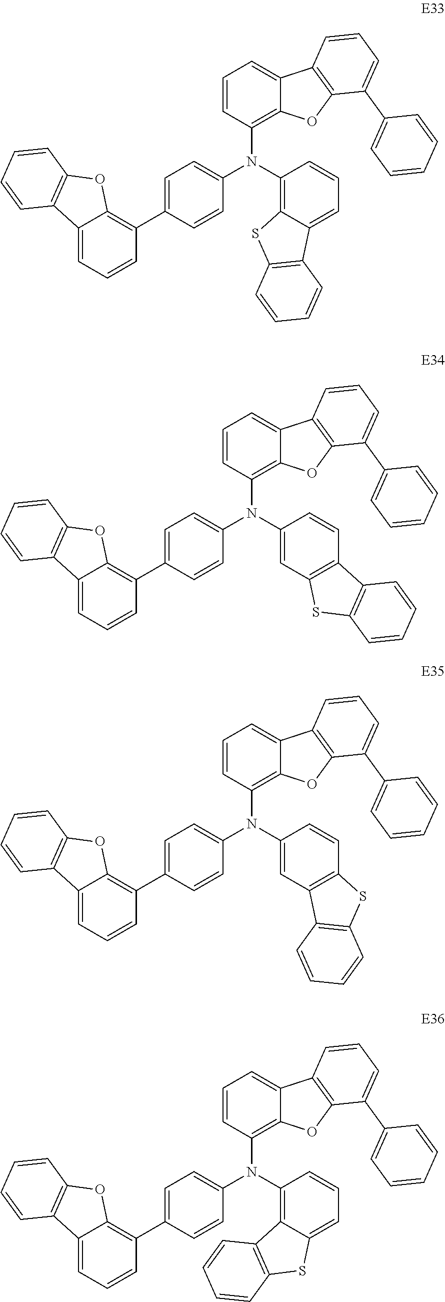 [SMILES: c1ccc(-c2cccc3c2oc2c(N(c4ccc(-c5cccc6c5oc5ccccc56)cc4)c4cccc5c4sc4ccccc45)cccc23)cc1 ; c1ccc(-c2cccc3c2oc2c(N(c4ccc(-c5cccc6c5oc5ccccc56)cc4)c4ccc5c(c4)sc4ccccc45)cccc23)cc1 ; c1ccc(-c2cccc3c2oc2c(N(c4ccc(-c5cccc6c5oc5ccccc56)cc4)c4ccc5sc6ccccc6c5c4)cccc23)cc1 ; c1ccc(-c2cccc3c2oc2c(N(c4ccc(-c5cccc6c5oc5ccccc56)cc4)c4cccc5sc6ccccc6c45)cccc23)cc1]

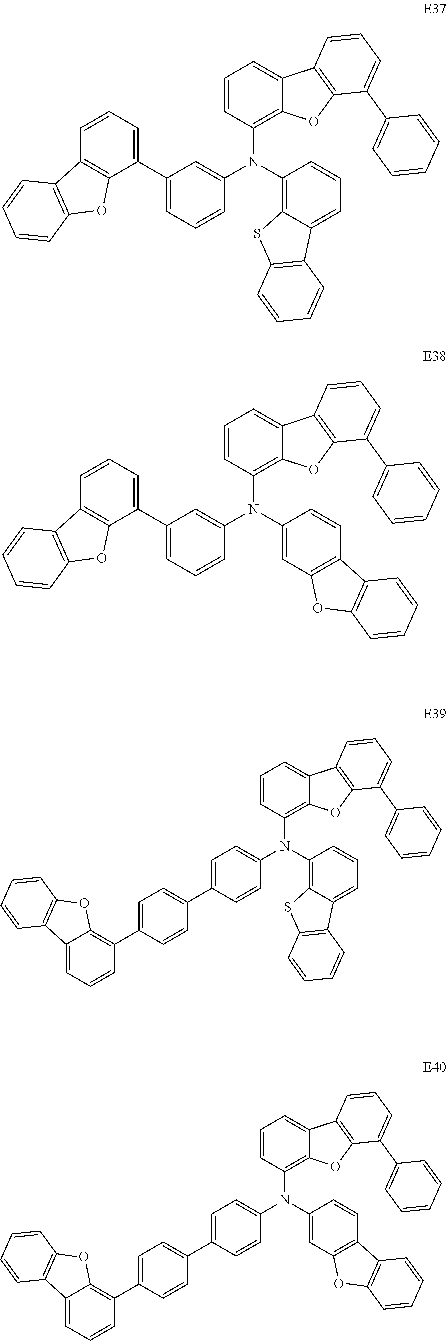 [SMILES: c1ccc(-c2cccc3c2oc2c(N(c4cccc(-c5cccc6c5oc5ccccc56)c4)c4cccc5c4sc4ccccc45)cccc23)cc1 ; c1ccc(-c2cccc3c2oc2c(N(c4cccc(-c5cccc6c5oc5ccccc56)c4)c4ccc5c(c4)oc4ccccc45)cccc23)cc1 ; c1ccc(-c2cccc3c2oc2c(N(c4ccc(-c5ccc(-c6cccc7c6oc6ccccc67)cc5)cc4)c4cccc5c4sc4ccccc45)cccc23)cc1 ; c1ccc(-c2cccc3c2oc2c(N(c4ccc(-c5ccc(-c6cccc7c6oc6ccccc67)cc5)cc4)c4ccc5c(c4)oc4ccccc45)cccc23)cc1]

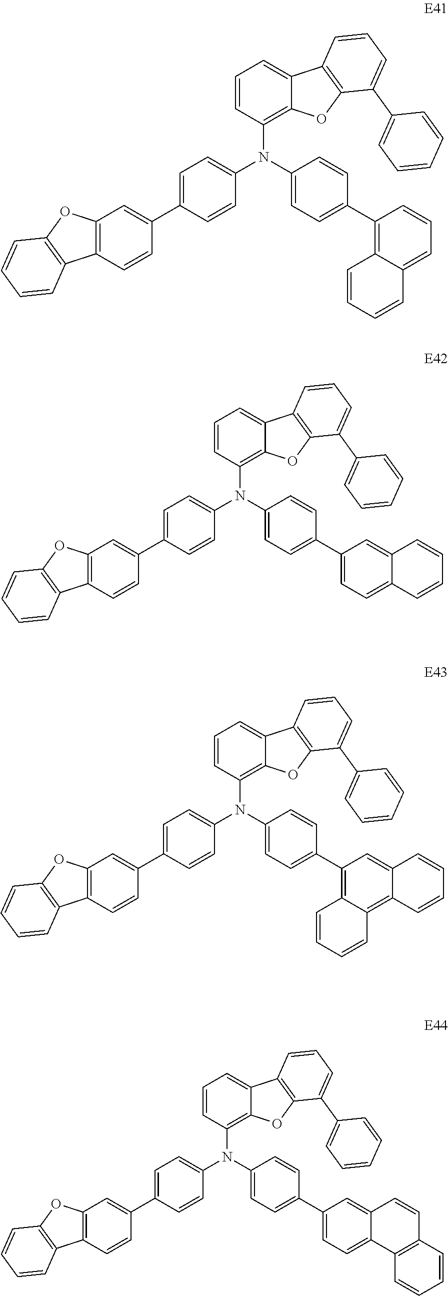 [SMILES: c1ccc(-c2cccc3c2oc2c(N(c4ccc(-c5ccc6c(c5)oc5ccccc56)cc4)c4ccc(-c5cccc6ccccc56)cc4)cccc23)cc1 ; c1ccc(-c2cccc3c2oc2c(N(c4ccc(-c5ccc6ccccc6c5)cc4)c4ccc(-c5ccc6c(c5)oc5ccccc56)cc4)cccc23)cc1 ; c1ccc(-c2cccc3c2oc2c(N(c4ccc(-c5ccc6c(c5)oc5ccccc56)cc4)c4ccc(-c5cc6ccccc6c6ccccc56)cc4)cccc23)cc1 ; c1ccc(-c2cccc3c2oc2c(N(c4ccc(-c5ccc6c(ccc7ccccc76)c5)cc4)c4ccc(-c5ccc6c(c5)oc5ccccc56)cc4)cccc23)cc1]

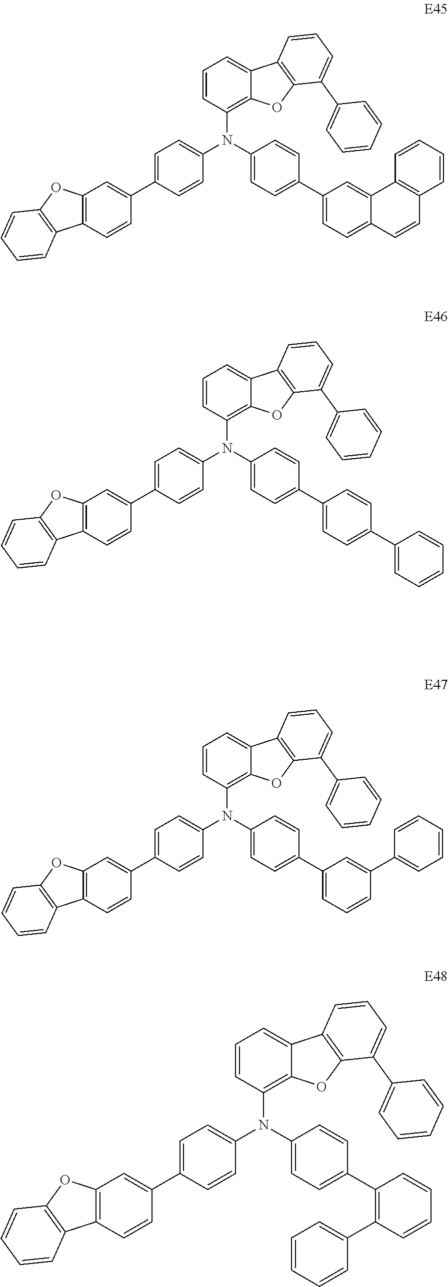 [SMILES: c1ccc(-c2cccc3c2oc2c(N(c4ccc(-c5ccc6c(c5)oc5ccccc56)cc4)c4ccc(-c5ccc6ccc7ccccc7c6c5)cc4)cccc23)cc1 ; c1ccc(-c2ccc(-c3ccc(N(c4ccc(-c5ccc6c(c5)oc5ccccc56)cc4)c4cccc5c4oc4c(-c6ccccc6)cccc45)cc3)cc2)cc1 ; c1ccc(-c2cccc(-c3ccc(N(c4ccc(-c5ccc6c(c5)oc5ccccc56)cc4)c4cccc5c4oc4c(-c6ccccc6)cccc45)cc3)c2)cc1 ; c1ccc(-c2ccccc2-c2ccc(N(c3ccc(-c4ccc5c(c4)oc4ccccc45)cc3)c3cccc4c3oc3c(-c5ccccc5)cccc34)cc2)cc1]

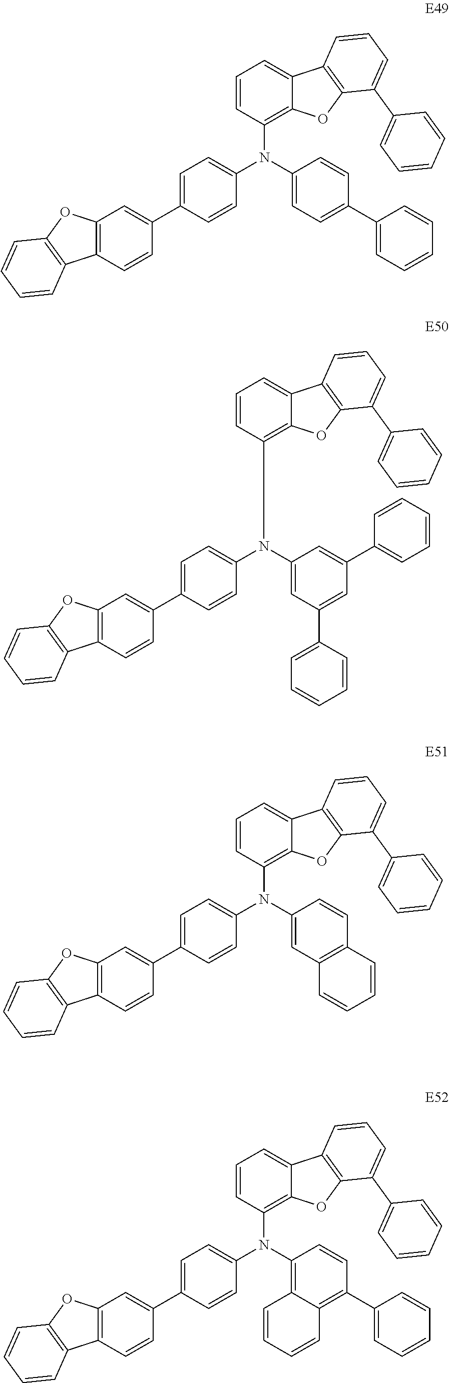 [SMILES: c1ccc(-c2ccc(N(c3ccc(-c4ccc5c(c4)oc4ccccc45)cc3)c3cccc4c3oc3c(-c5ccccc5)cccc34)cc2)cc1 ; c1ccc(-c2cc(-c3ccccc3)cc(N(c3ccc(-c4ccc5c(c4)oc4ccccc45)cc3)c3cccc4c3oc3c(-c5ccccc5)cccc34)c2)cc1 ; c1ccc(-c2cccc3c2oc2c(N(c4ccc(-c5ccc6c(c5)oc5ccccc56)cc4)c4ccc5ccccc5c4)cccc23)cc1 ; c1ccc(-c2ccc(N(c3ccc(-c4ccc5c(c4)oc4ccccc45)cc3)c3cccc4c3oc3c(-c5ccccc5)cccc34)c3ccccc23)cc1]

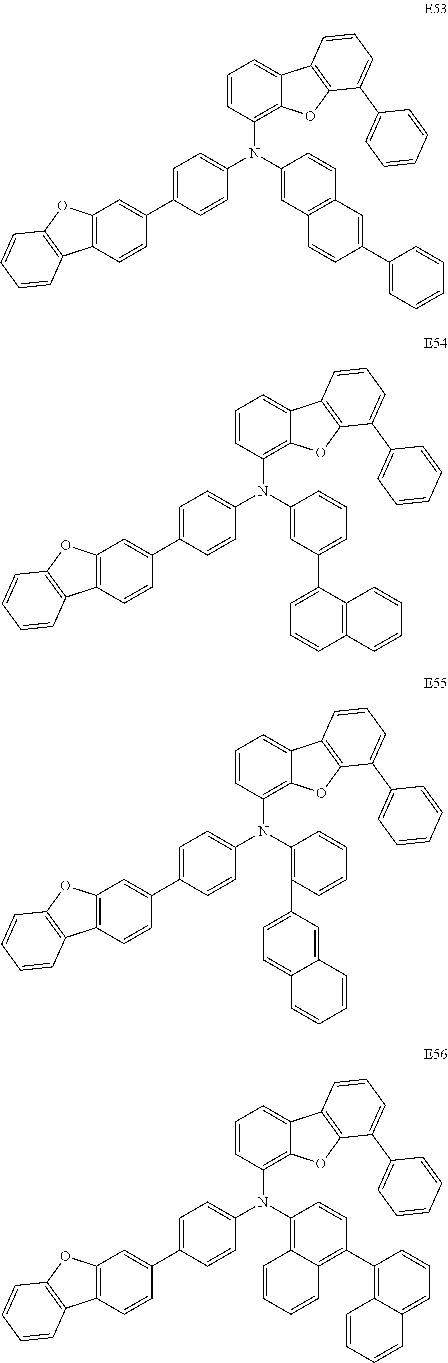 [SMILES: c1ccc(-c2ccc3cc(N(c4ccc(-c5ccc6c(c5)oc5ccccc56)cc4)c4cccc5c4oc4c(-c6ccccc6)cccc45)ccc3c2)cc1 ; c1ccc(-c2cccc3c2oc2c(N(c4ccc(-c5ccc6c(c5)oc5ccccc56)cc4)c4cccc(-c5cccc6ccccc56)c4)cccc23)cc1 ; c1ccc(-c2cccc3c2oc2c(N(c4ccc(-c5ccc6c(c5)oc5ccccc56)cc4)c4ccccc4-c4ccc5ccccc5c4)cccc23)cc1 ; c1ccc(-c2cccc3c2oc2c(N(c4ccc(-c5ccc6c(c5)oc5ccccc56)cc4)c4ccc(-c5cccc6ccccc56)c5ccccc45)cccc23)cc1]

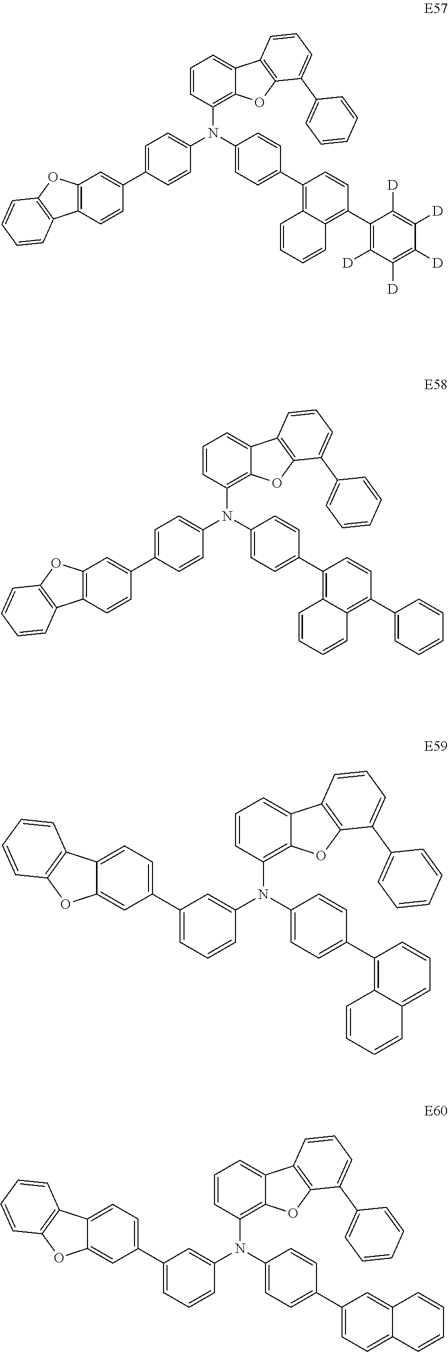 [SMILES: [2H]c1c([2H])c([2H])c(-c2ccc(-c3ccc(N(c4ccc(-c5ccc6c(c5)oc5ccccc56)cc4)c4cccc5c4oc4c(-c6ccccc6)cccc45)cc3)c3ccccc23)c([2H])c1[2H] ; c1ccc(-c2ccc(-c3ccc(N(c4ccc(-c5ccc6c(c5)oc5ccccc56)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5ccc6c(c5)oc5ccccc56)c4)cccc23)cc1 ; c1ccc(-c2cccc3c2oc2c(N(c4ccc(-c5ccc6ccccc6c5)cc4)c4cccc(-c5ccc6c(c5)oc5ccccc56)c4)cccc23)cc1]

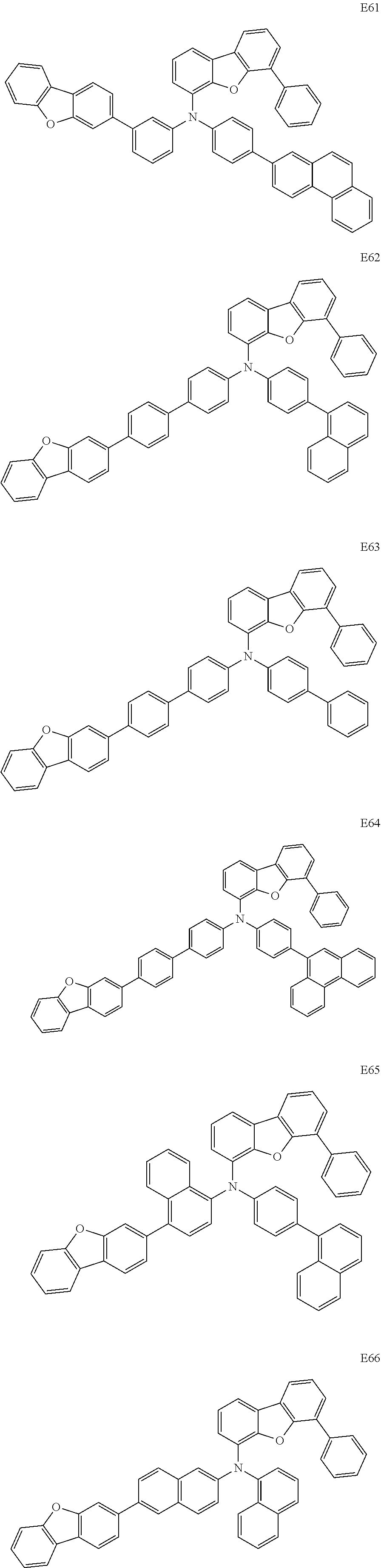

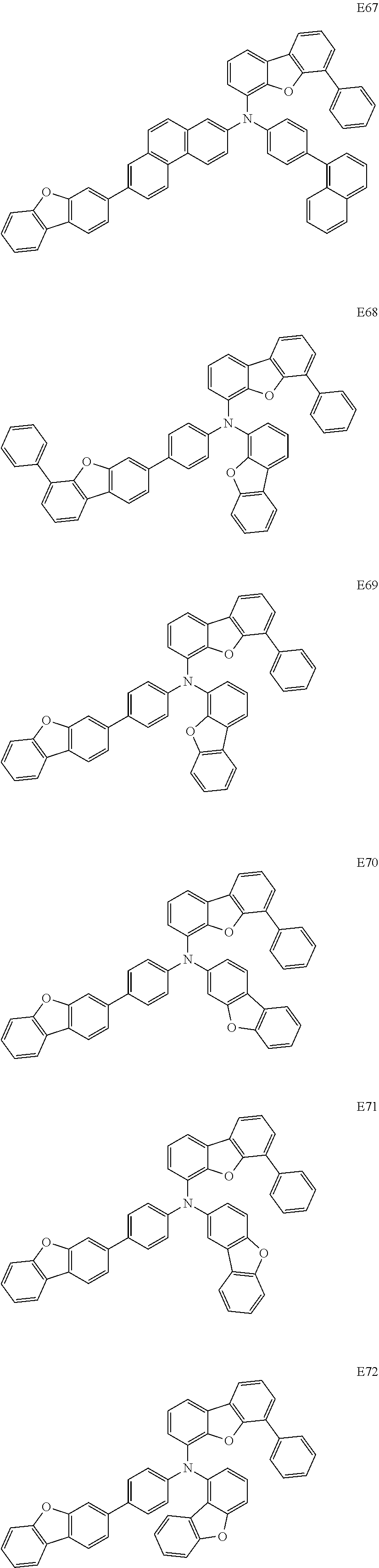

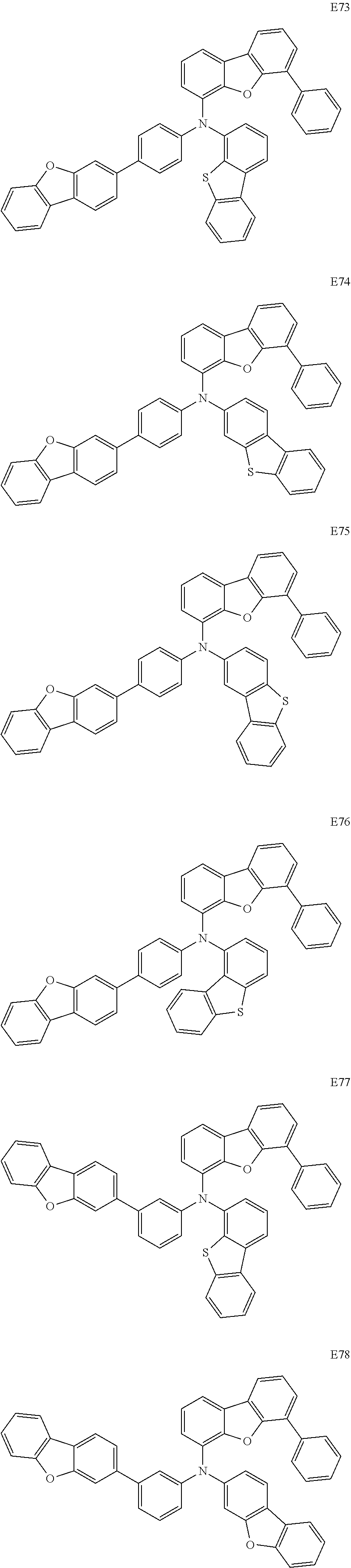

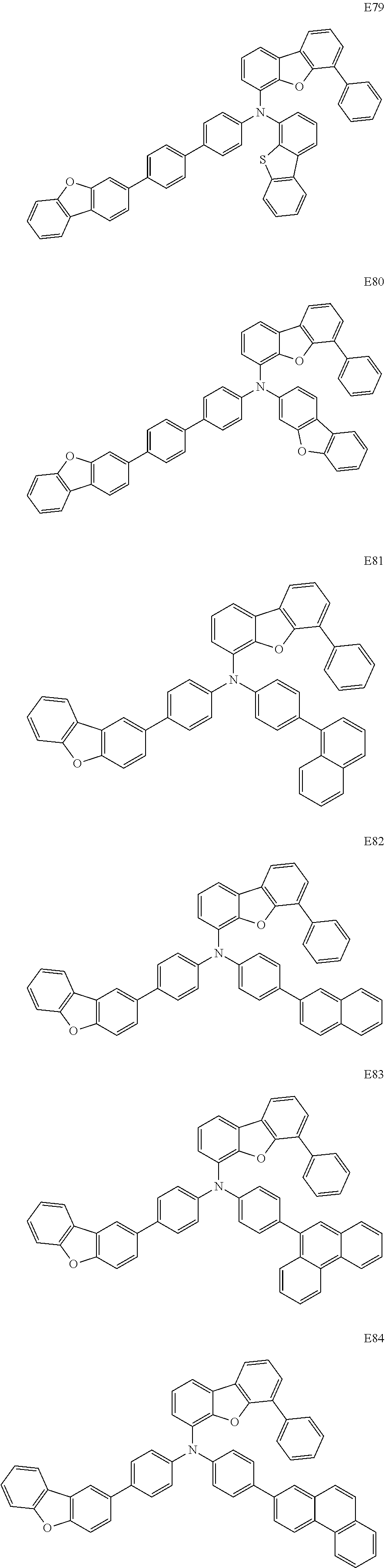

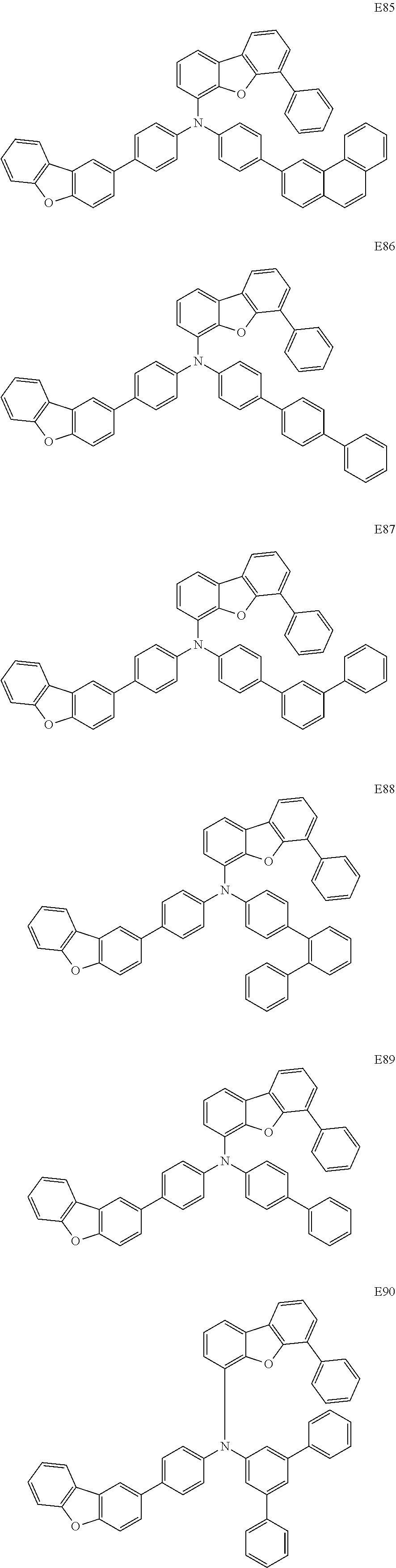

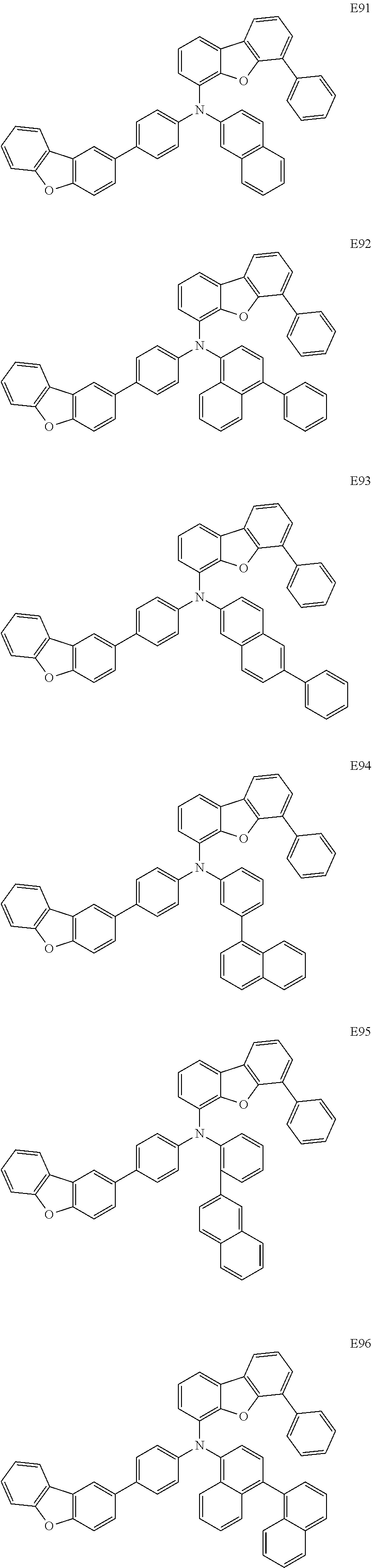

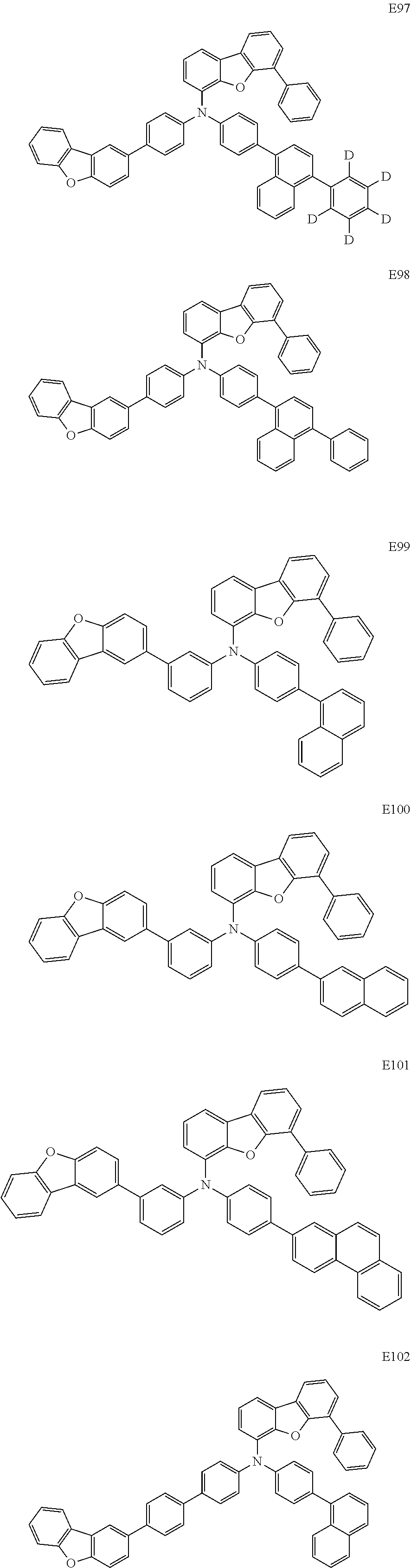

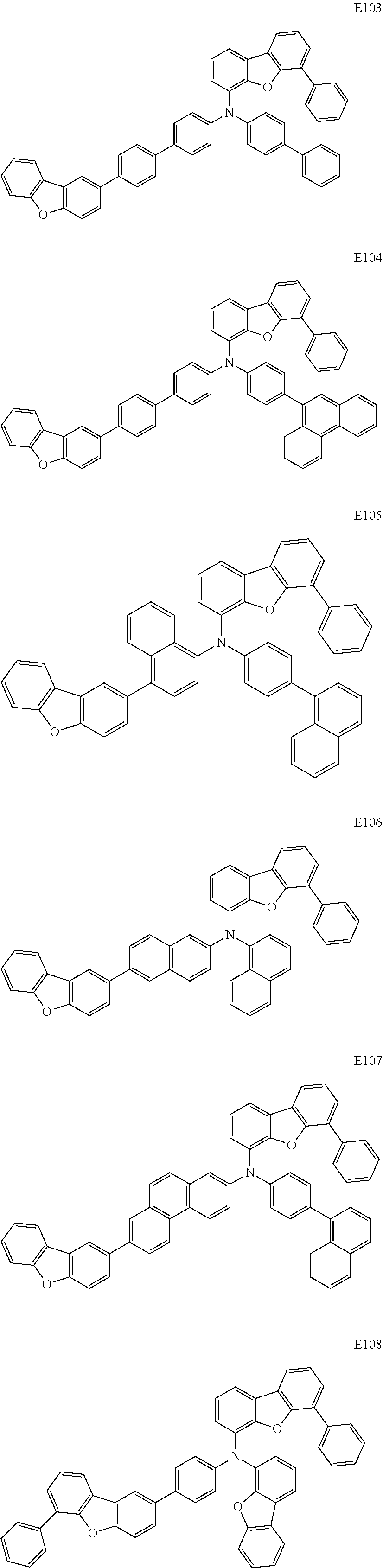

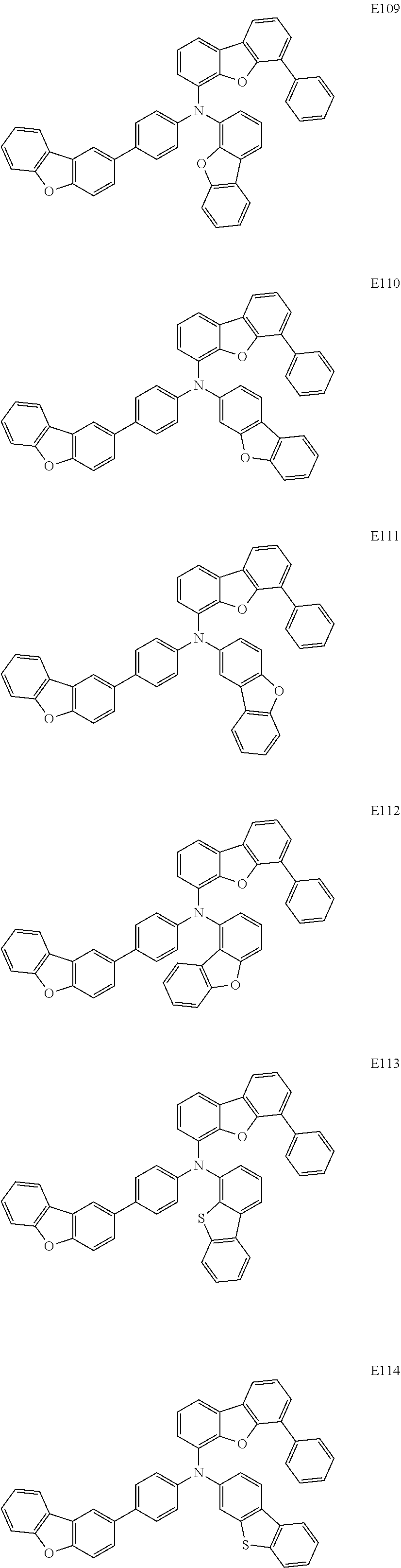

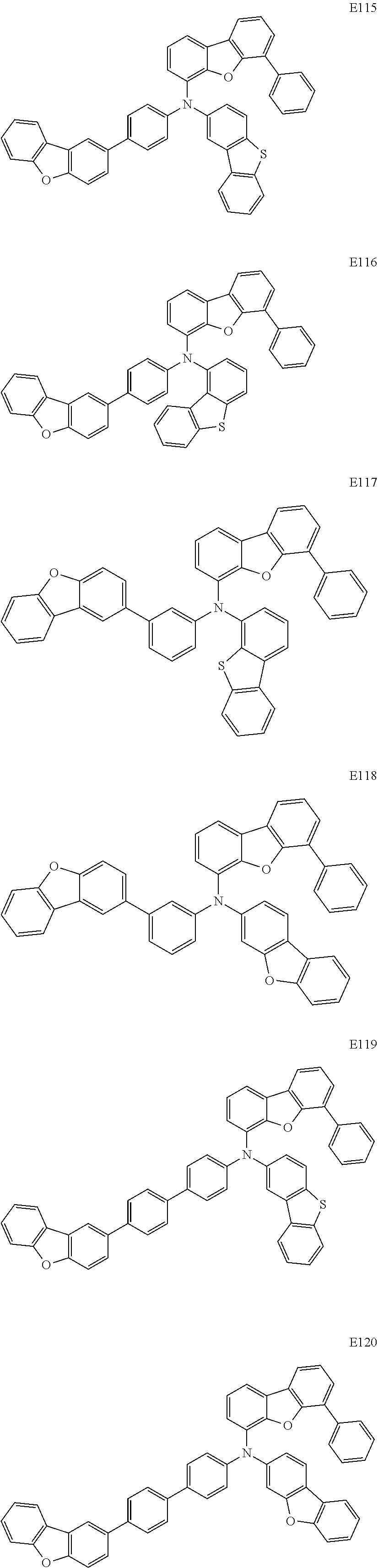

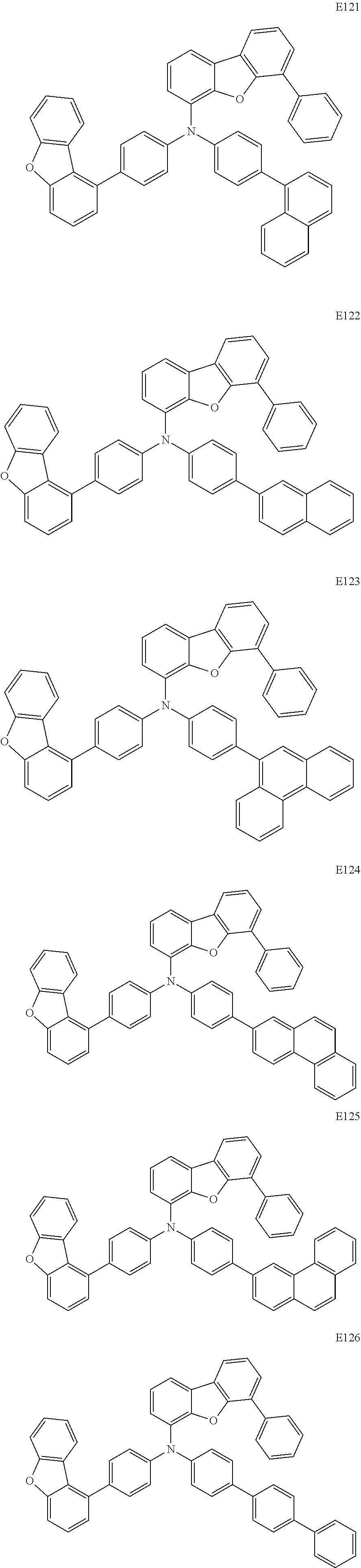

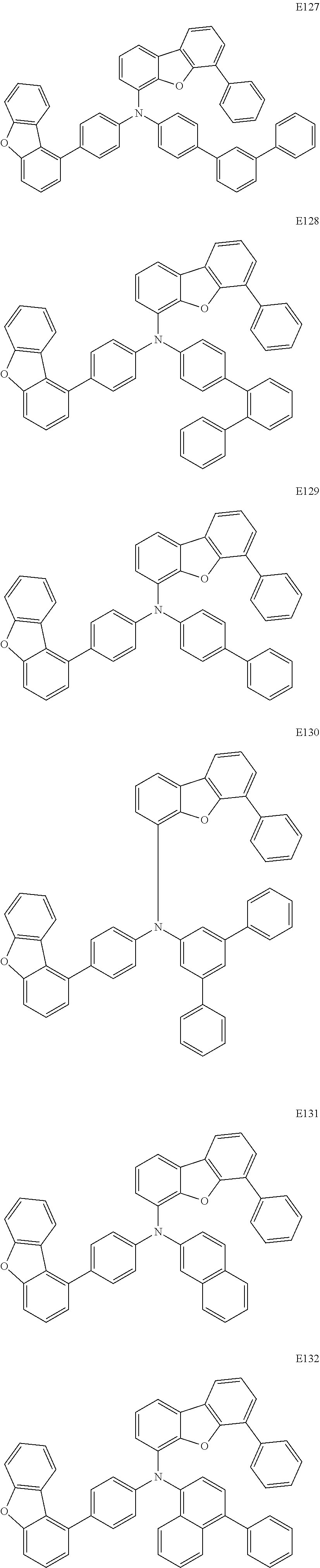

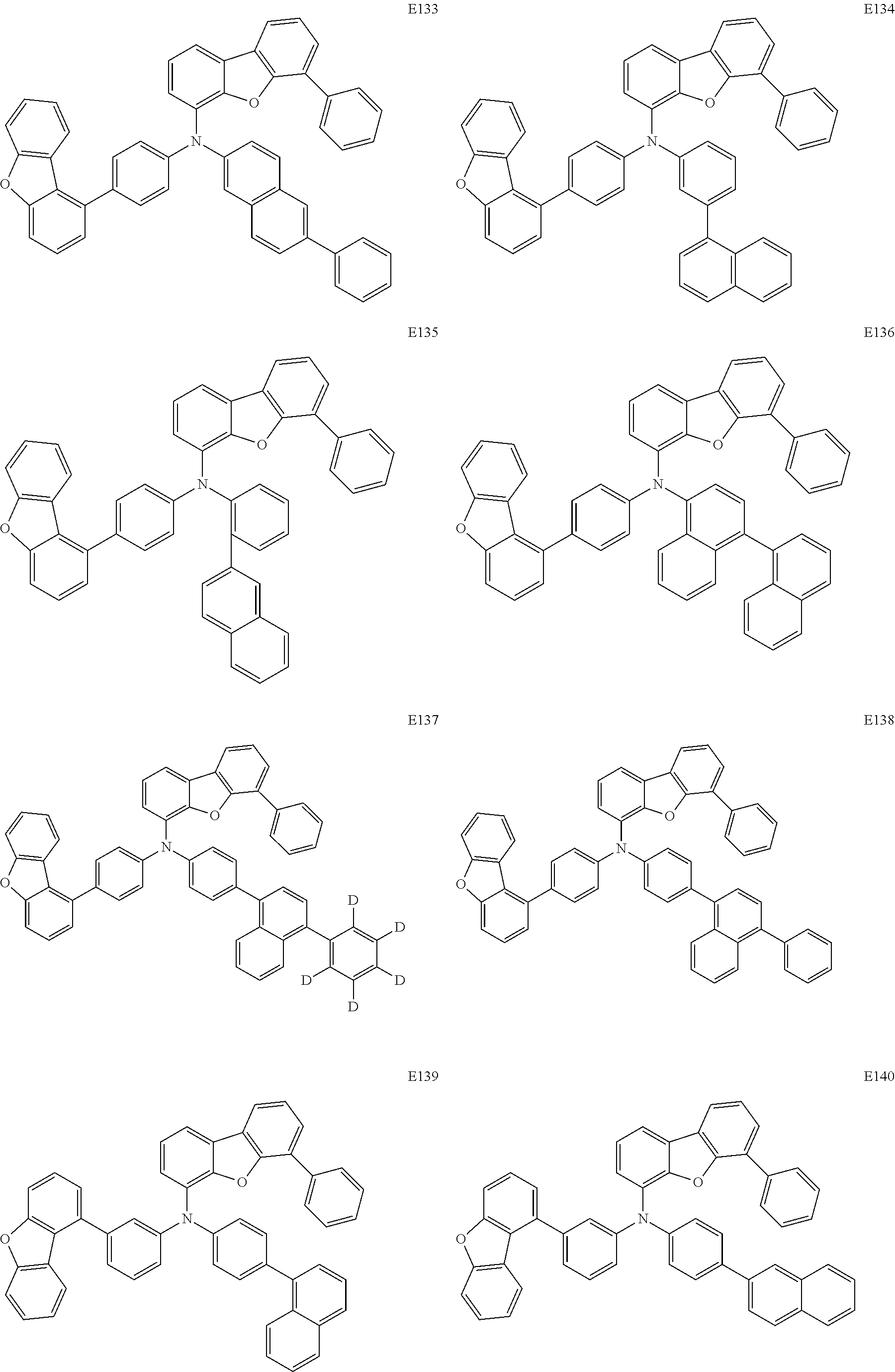

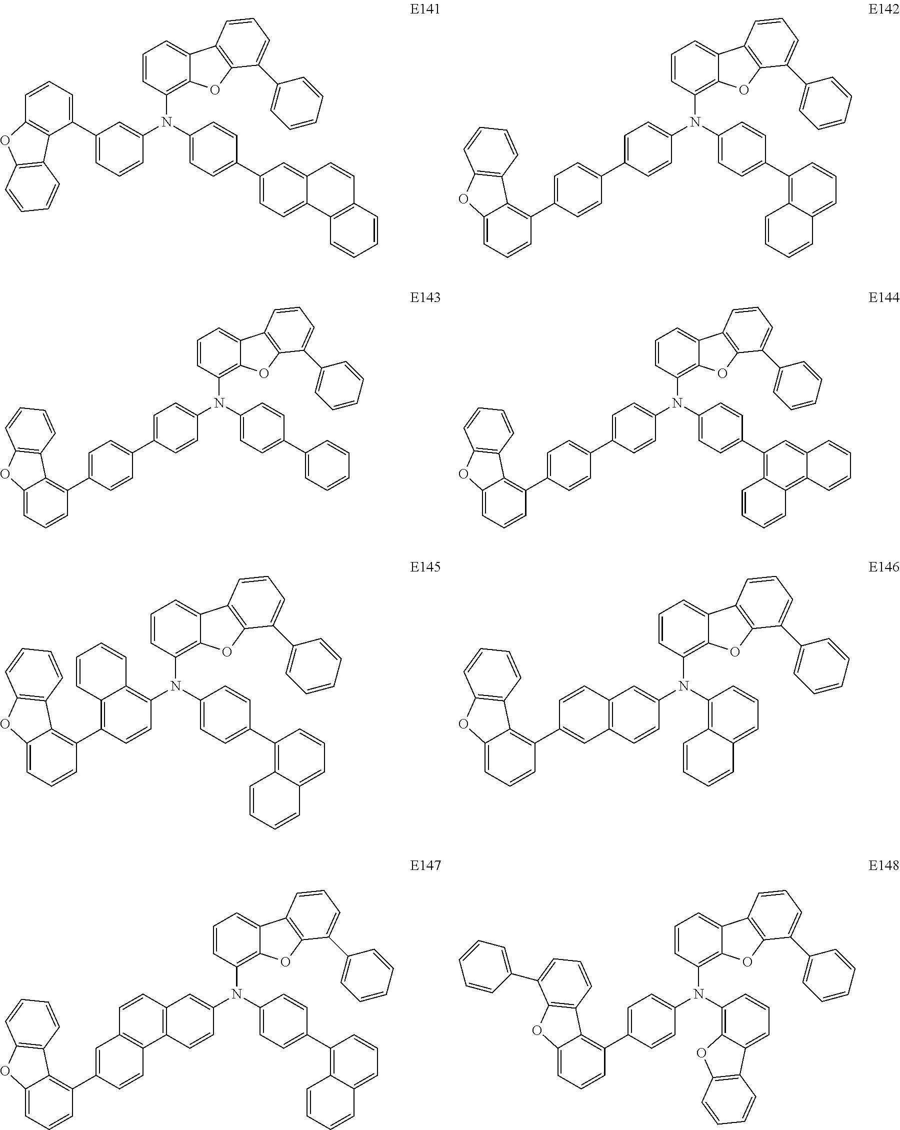

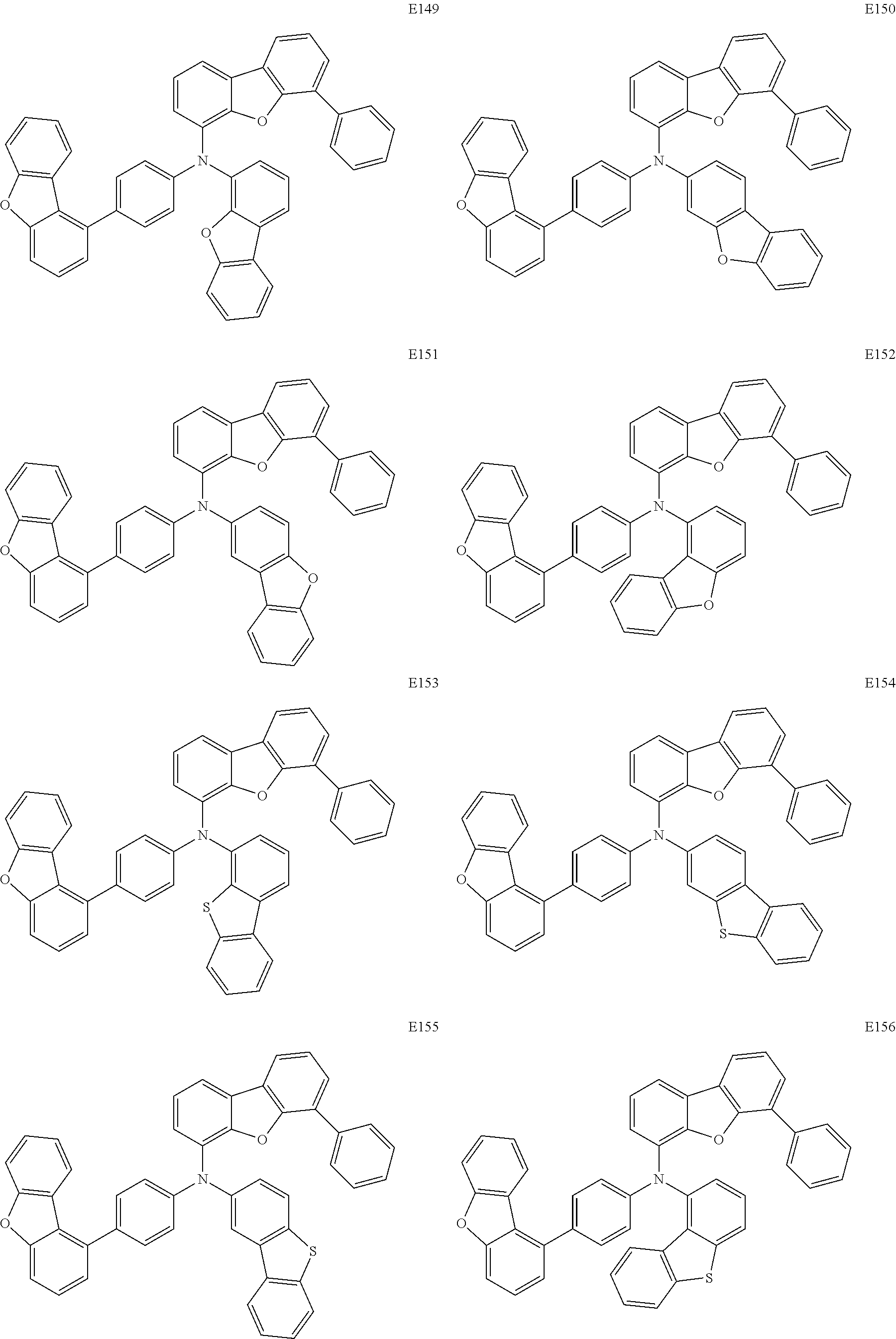

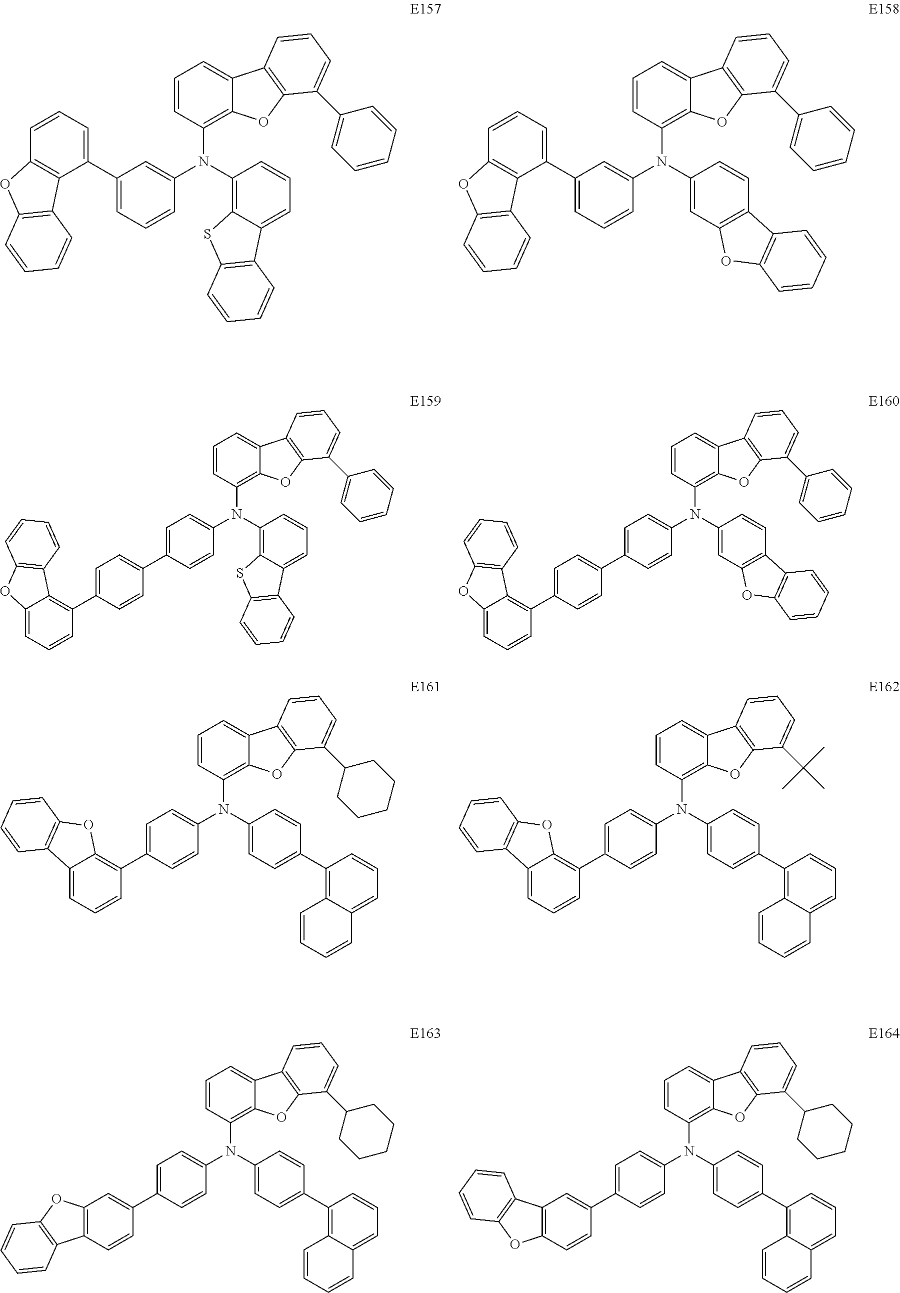

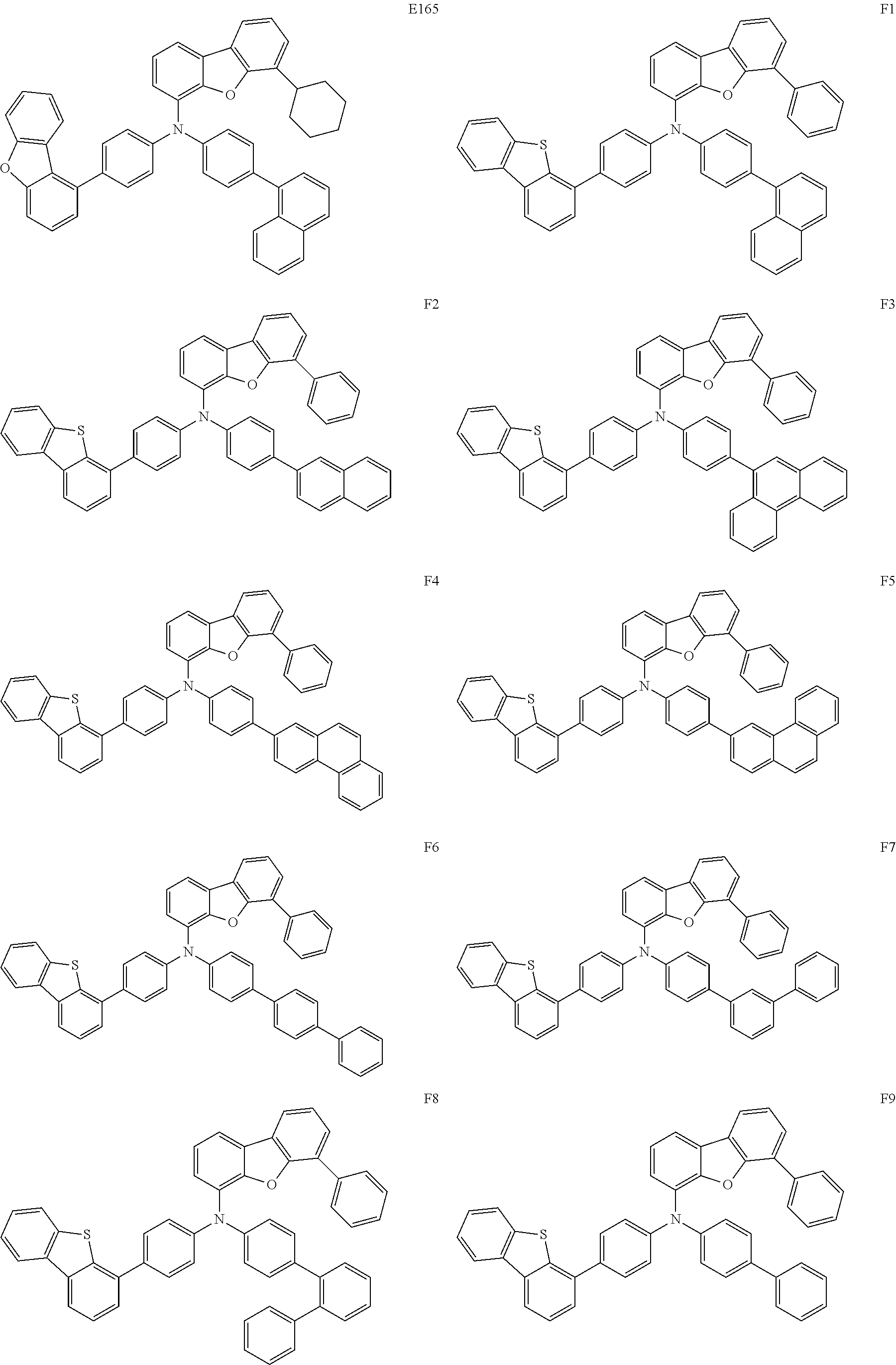

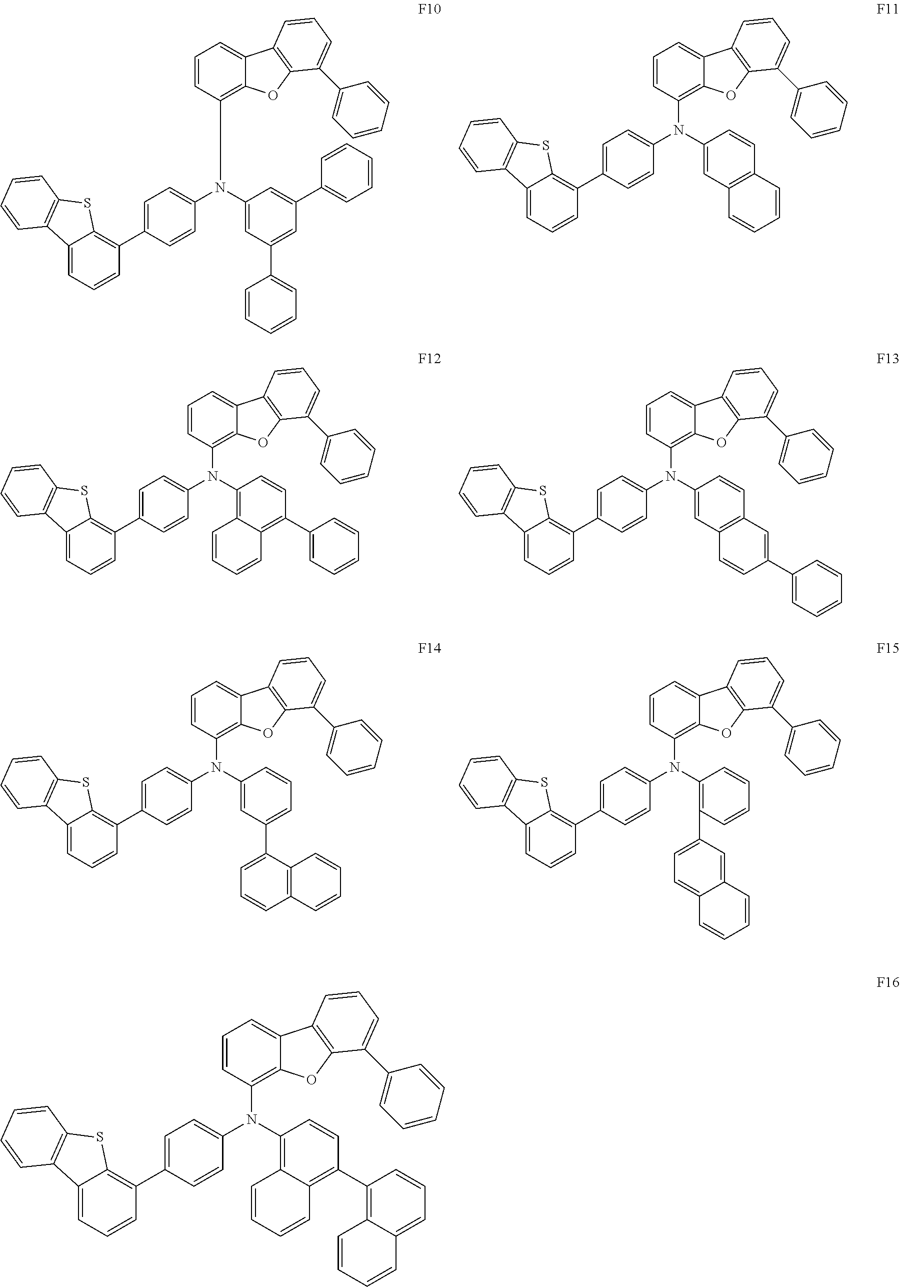

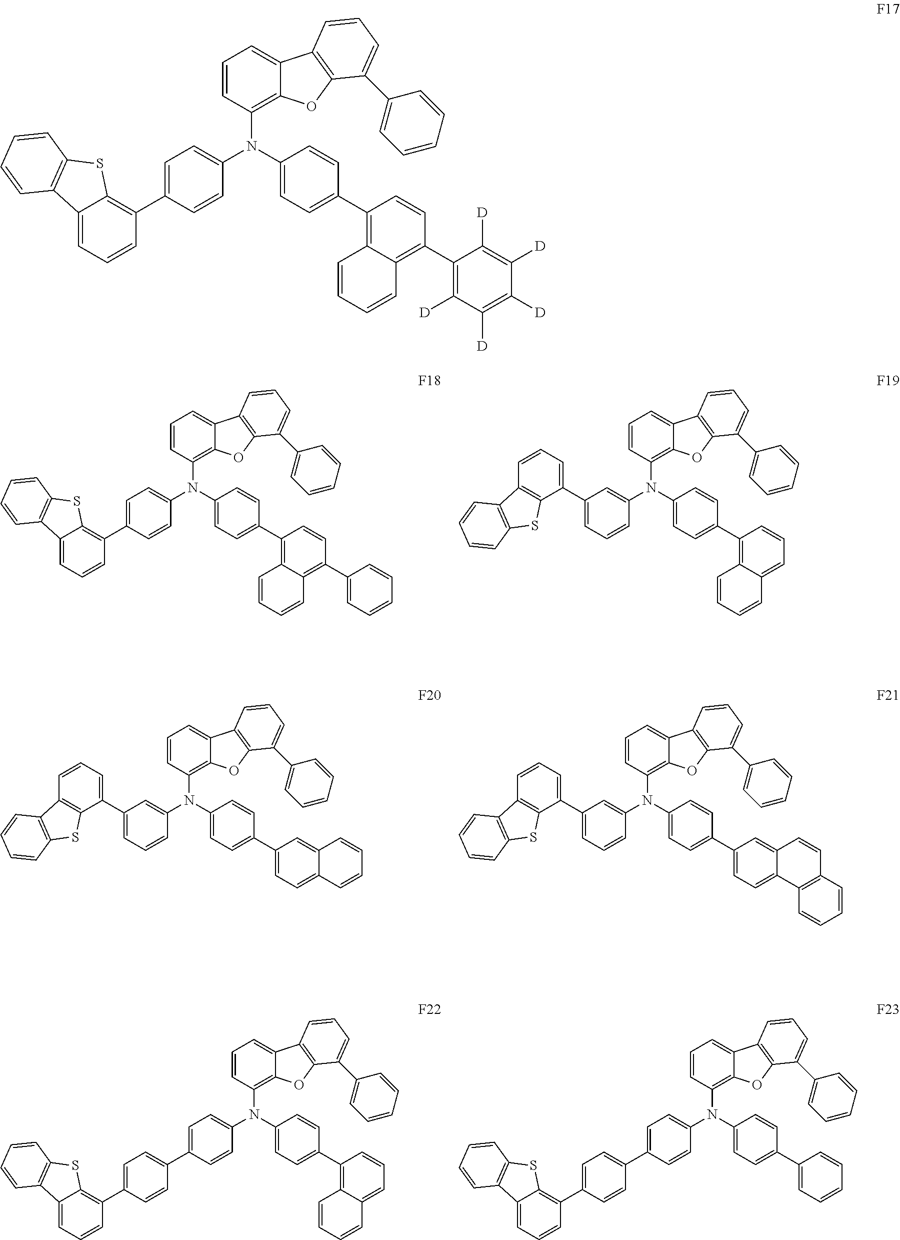

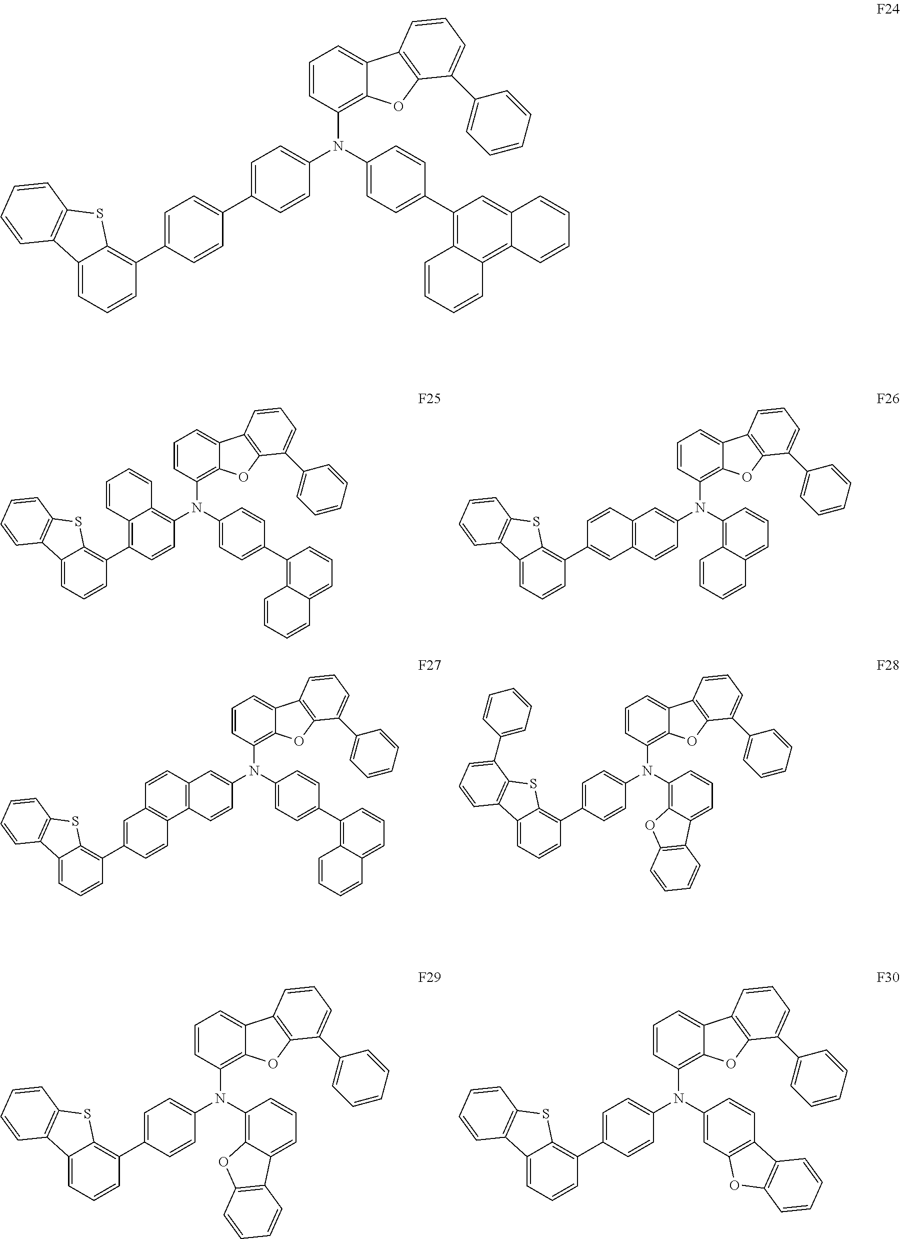

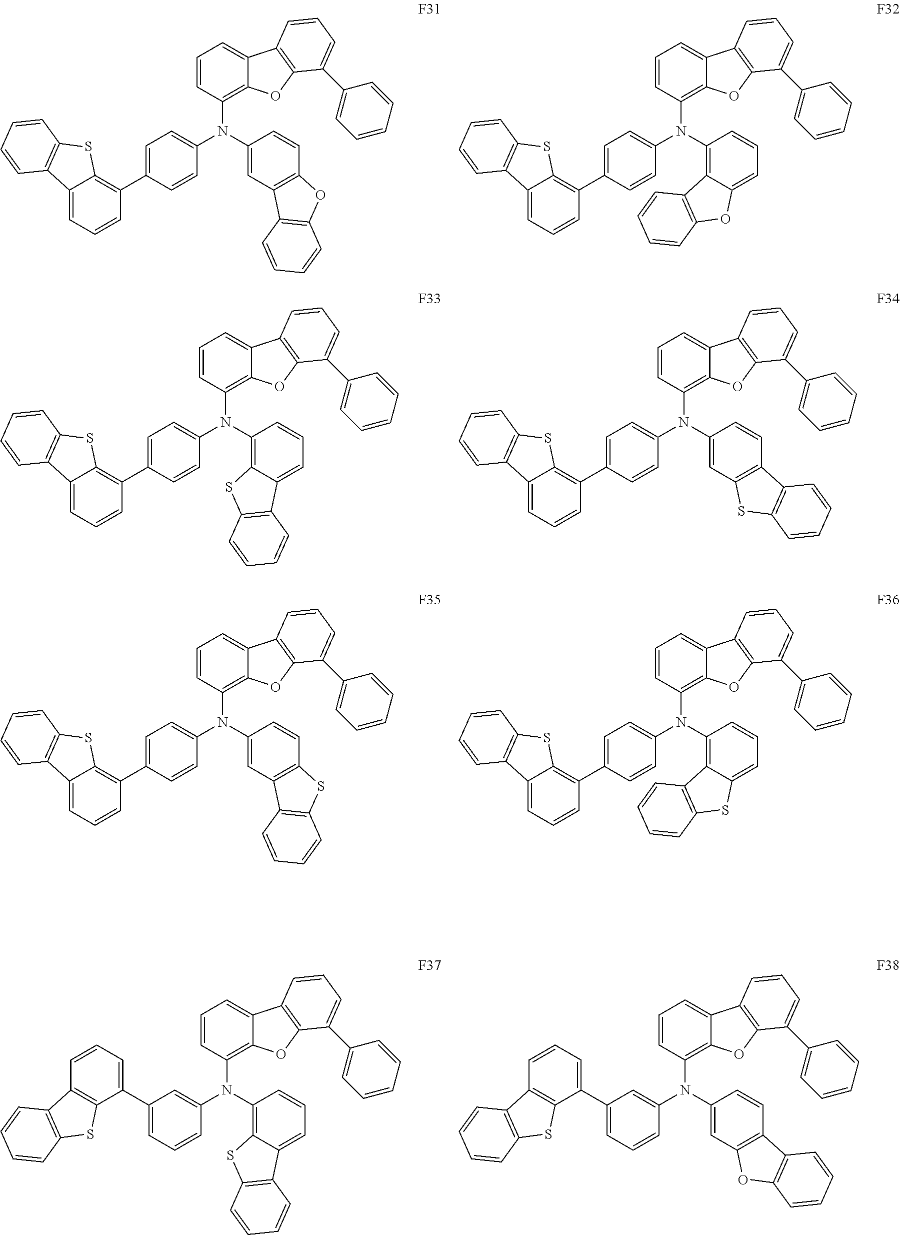

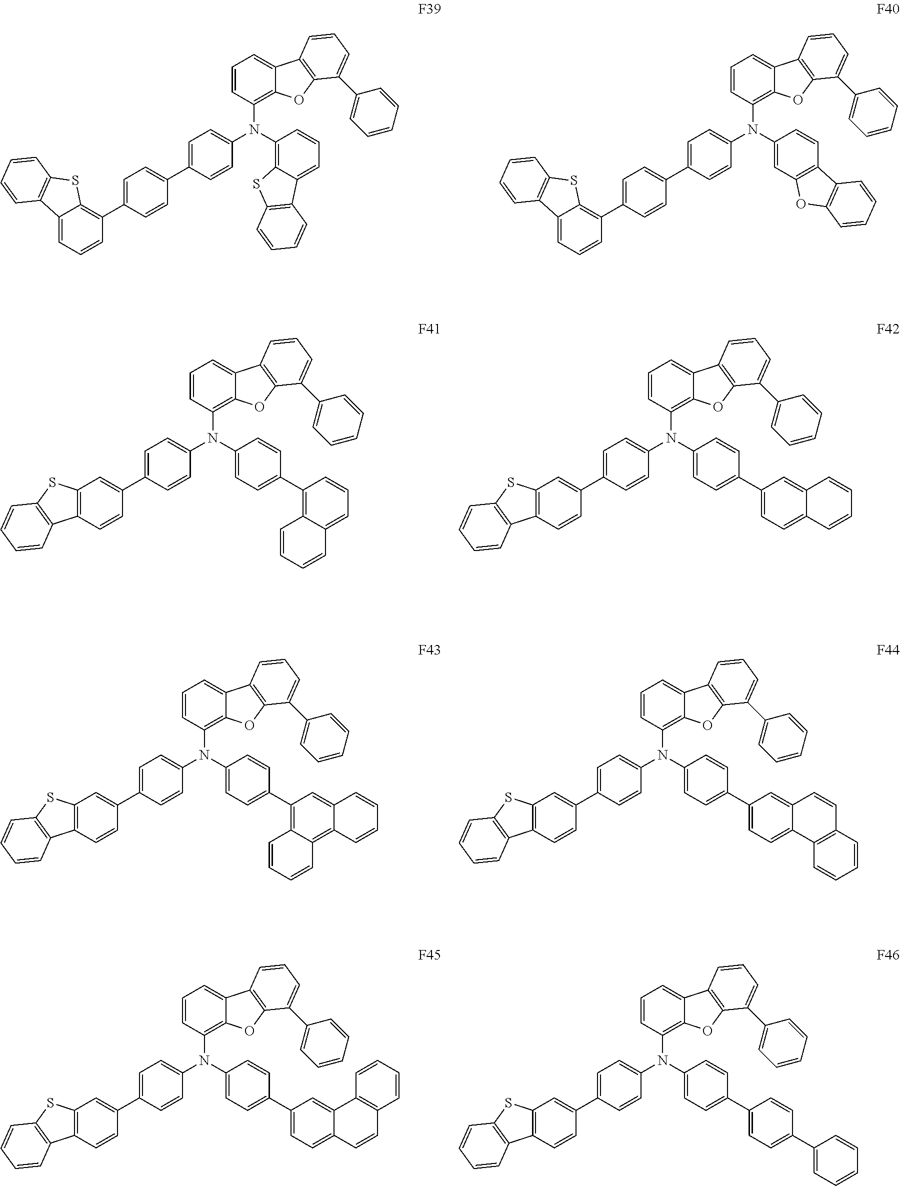

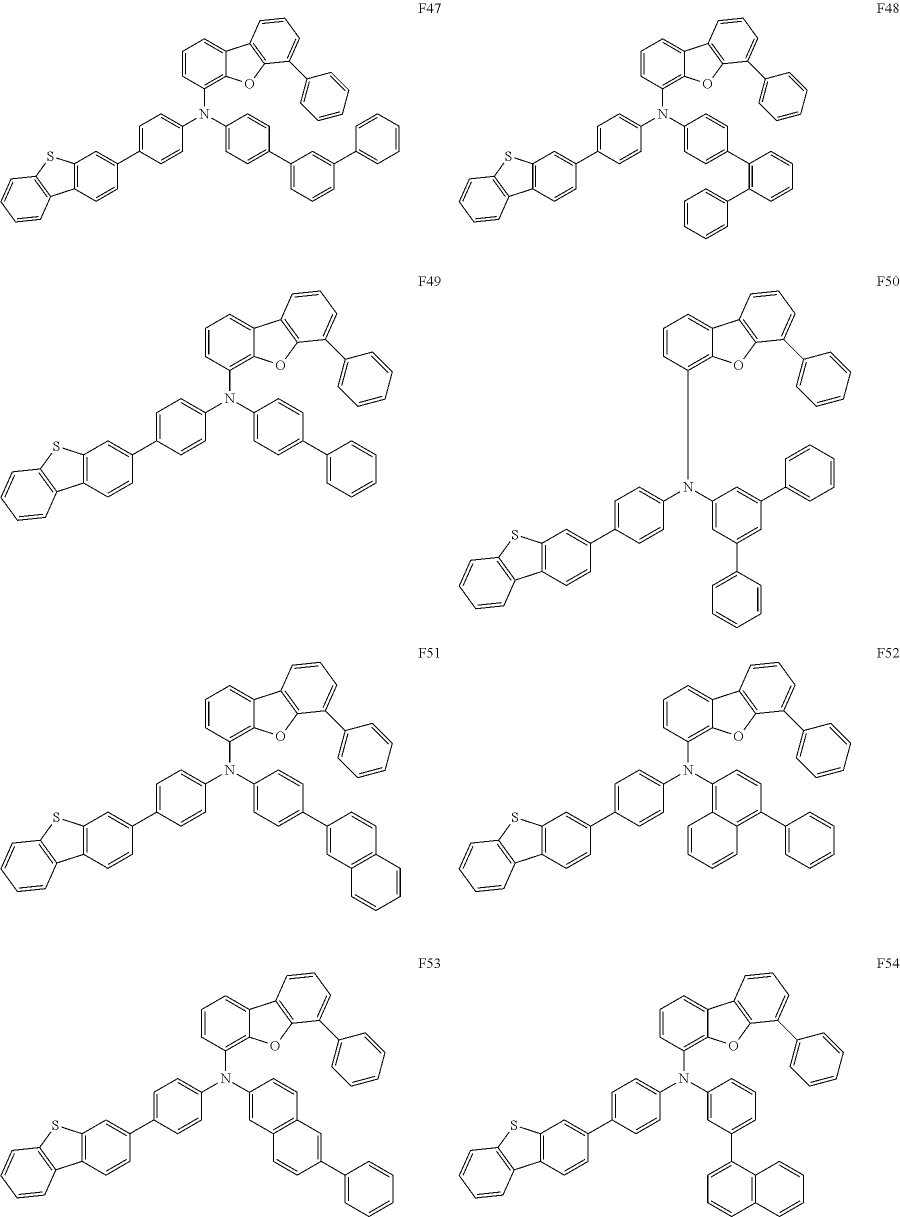

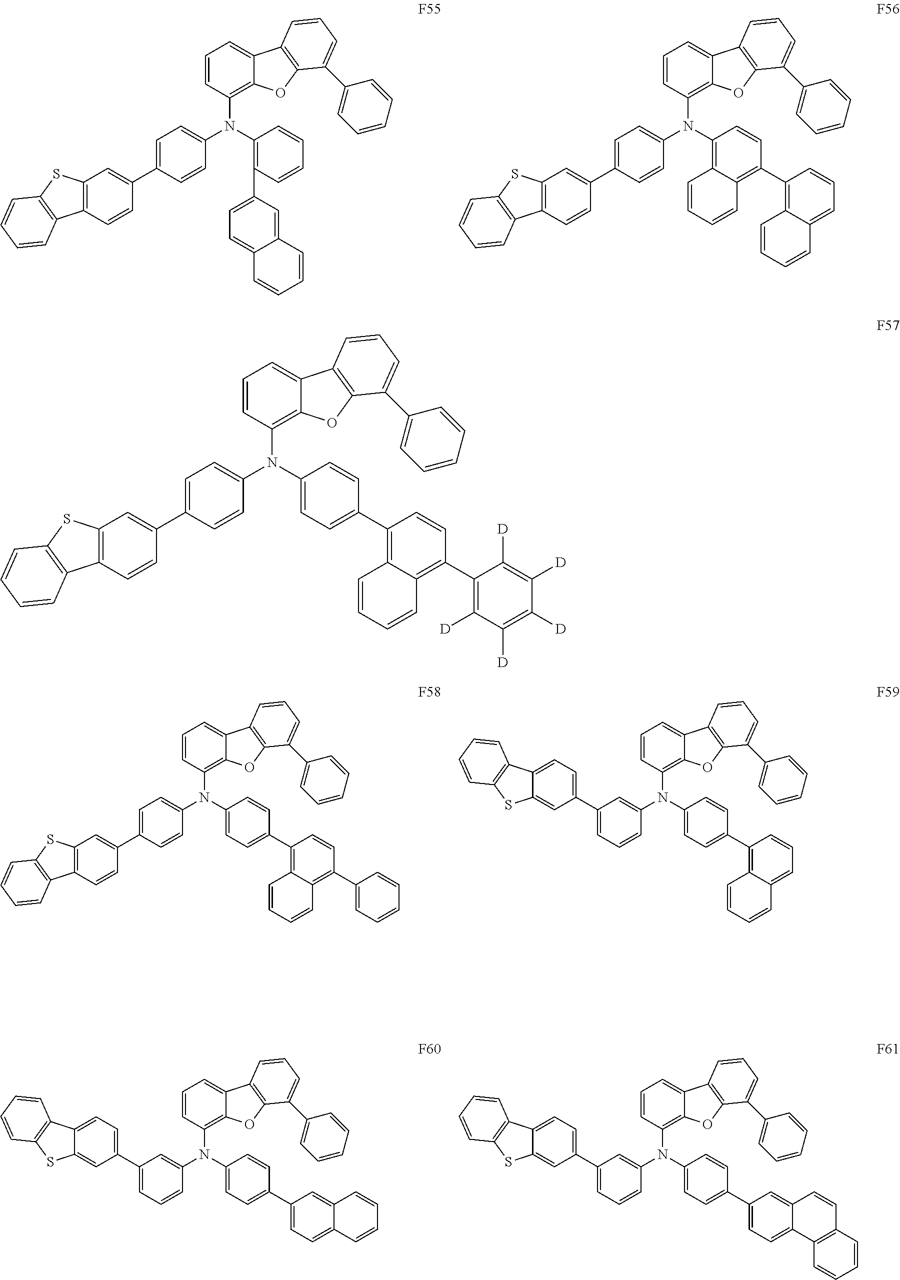

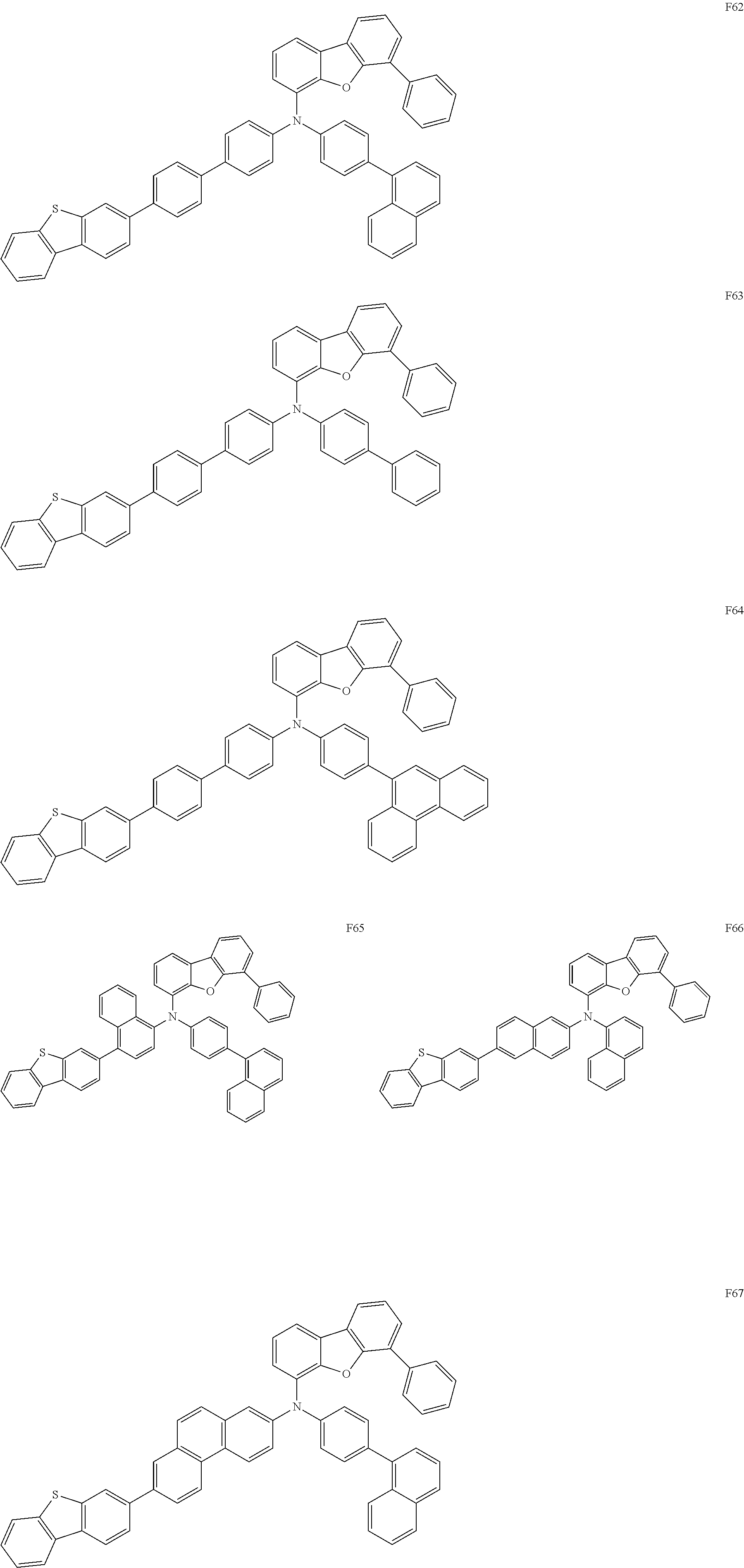

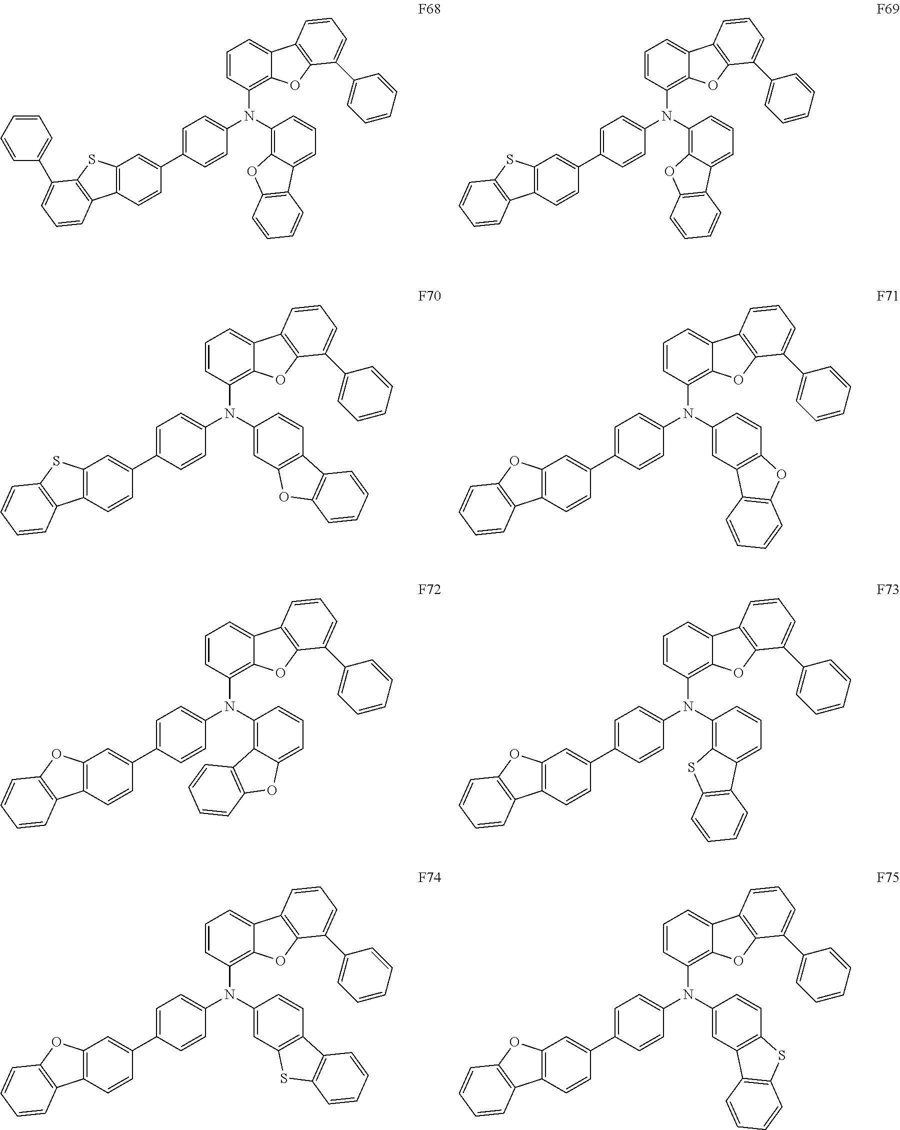

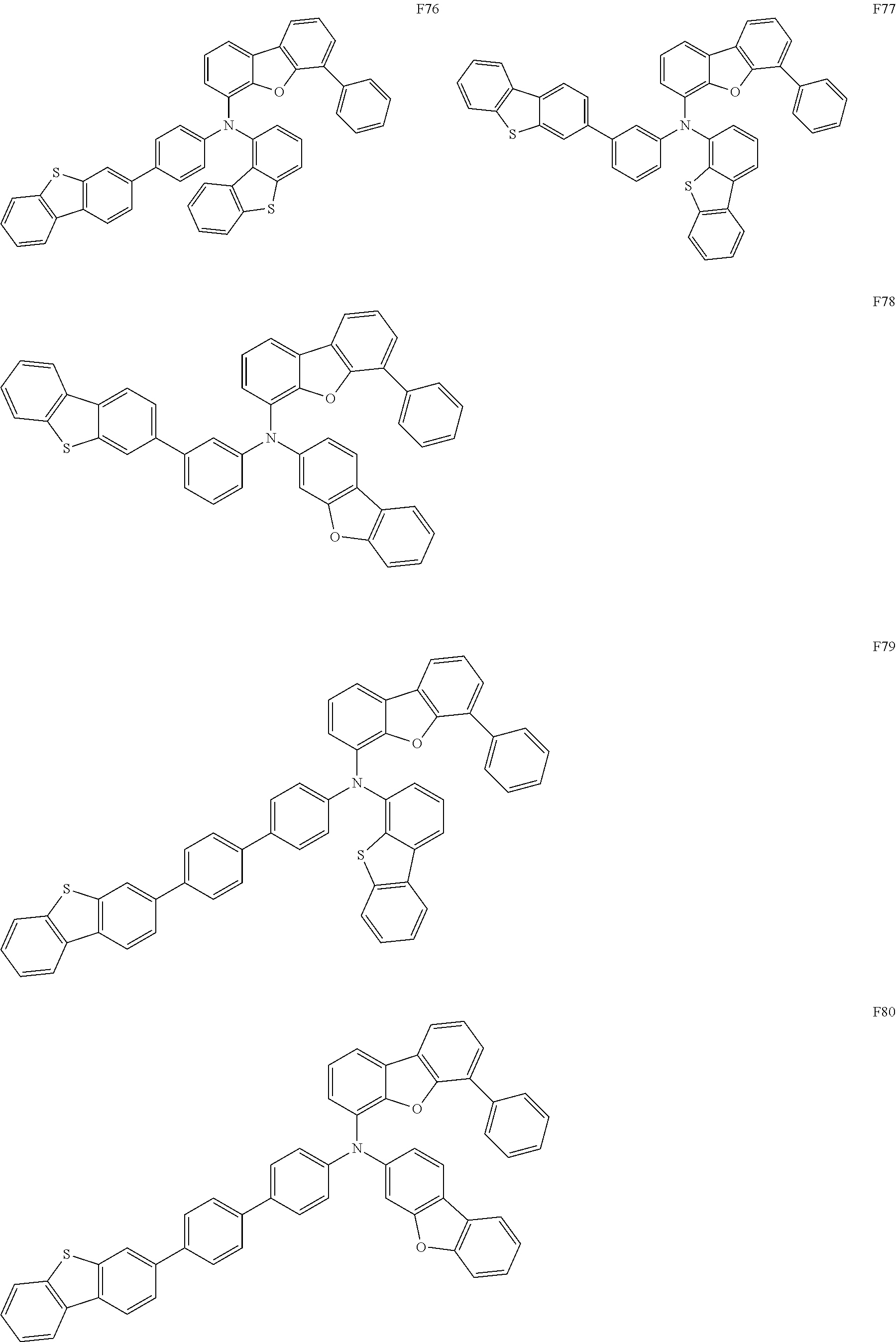 [SMILES: c1ccc(-c2cccc3c2oc2c(N(c4ccc(-c5ccc6c(c5)sc5ccccc56)cc4)c4cccc5sc6ccccc6c45)cccc23)cc1 ; c1ccc(-c2cccc3c2oc2c(N(c4cccc(-c5ccc6c(c5)sc5ccccc56)c4)c4cccc5c4sc4ccccc45)cccc23)cc1 ; c1ccc(-c2cccc3c2oc2c(N(c4cccc(-c5ccc6c(c5)sc5ccccc56)c4)c4ccc5c(c4)oc4ccccc45)cccc23)cc1 ; c1ccc(-c2cccc3c2oc2c(N(c4ccc(-c5ccc(-c6ccc7c(c6)sc6ccccc67)cc5)cc4)c4cccc5c4sc4ccccc45)cccc23)cc1 ; c1ccc(-c2cccc3c2oc2c(N(c4ccc(-c5ccc(-c6ccc7c(c6)sc6ccccc67)cc5)cc4)c4ccc5c(c4)oc4ccccc45)cccc23)cc1]

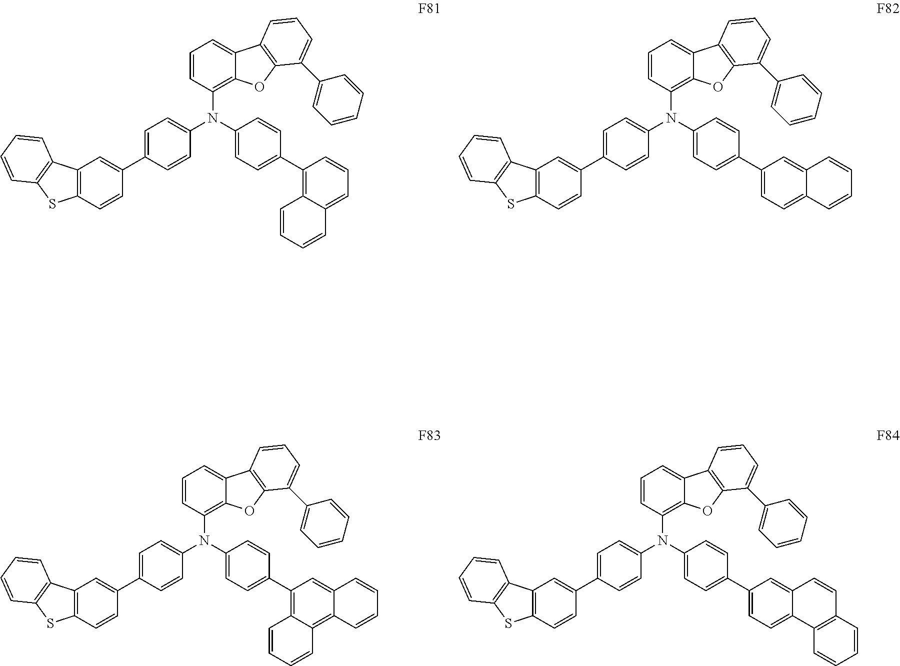 [SMILES: c1ccc(-c2cccc3c2oc2c(N(c4ccc(-c5ccc6sc7ccccc7c6c5)cc4)c4ccc(-c5cccc6ccccc56)cc4)cccc23)cc1 ; c1ccc(-c2cccc3c2oc2c(N(c4ccc(-c5ccc6ccccc6c5)cc4)c4ccc(-c5ccc6sc7ccccc7c6c5)cc4)cccc23)cc1 ; c1ccc(-c2cccc3c2oc2c(N(c4ccc(-c5ccc6sc7ccccc7c6c5)cc4)c4ccc(-c5cc6ccccc6c6ccccc56)cc4)cccc23)cc1 ; c1ccc(-c2cccc3c2oc2c(N(c4ccc(-c5ccc6c(ccc7ccccc76)c5)cc4)c4ccc(-c5ccc6sc7ccccc7c6c5)cc4)cccc23)cc1]

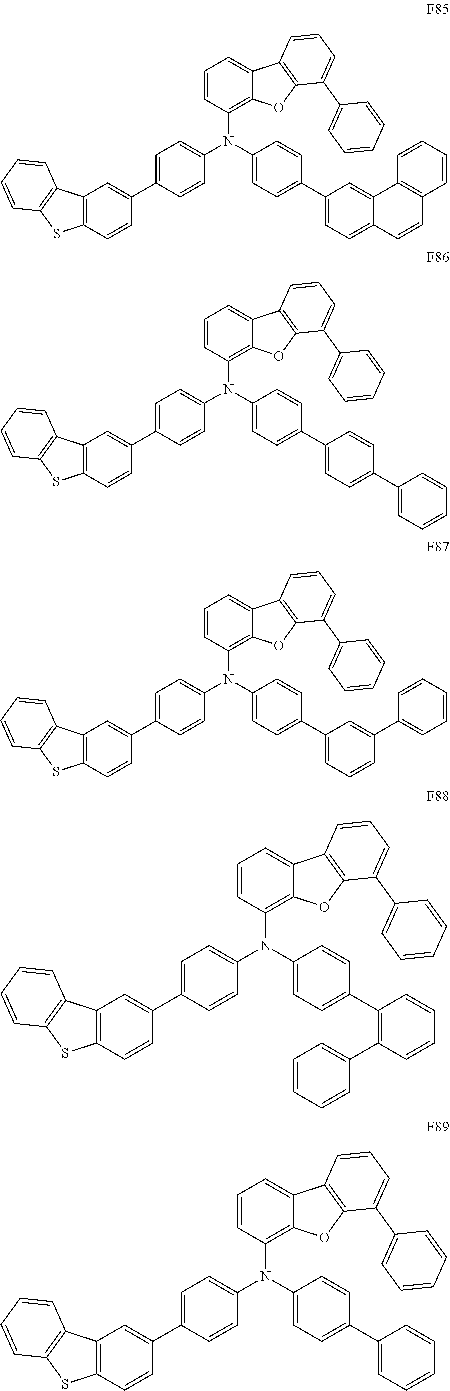 [SMILES: c1ccc(-c2cccc3c2oc2c(N(c4ccc(-c5ccc6ccc7ccccc7c6c5)cc4)c4ccc(-c5ccc6sc7ccccc7c6c5)cc4)cccc23)cc1 ; c1ccc(-c2ccc(-c3ccc(N(c4ccc(-c5ccc6sc7ccccc7c6c5)cc4)c4cccc5c4oc4c(-c6ccccc6)cccc45)cc3)cc2)cc1 ; c1ccc(-c2cccc(-c3ccc(N(c4ccc(-c5ccc6sc7ccccc7c6c5)cc4)c4cccc5c4oc4c(-c6ccccc6)cccc45)cc3)c2)cc1 ; c1ccc(-c2ccccc2-c2ccc(N(c3ccc(-c4ccc5sc6ccccc6c5c4)cc3)c3cccc4c3oc3c(-c5ccccc5)cccc34)cc2)cc1 ; c1ccc(-c2ccc(N(c3ccc(-c4ccc5sc6ccccc6c5c4)cc3)c3cccc4c3oc3c(-c5ccccc5)cccc34)cc2)cc1]

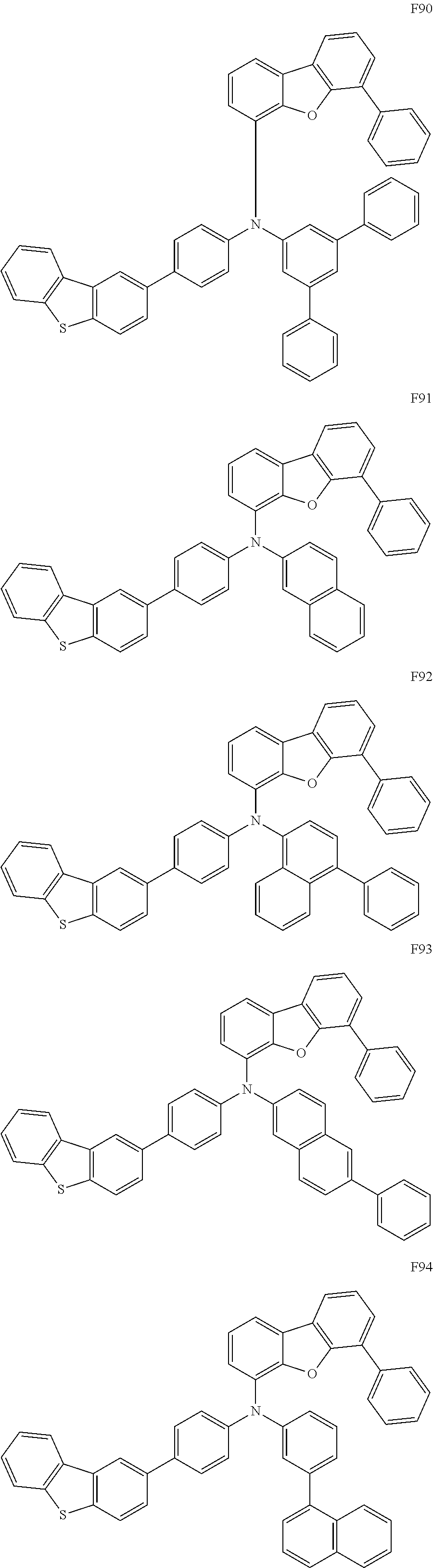 [SMILES: c1ccc(-c2cc(-c3ccccc3)cc(N(c3ccc(-c4ccc5sc6ccccc6c5c4)cc3)c3cccc4c3oc3c(-c5ccccc5)cccc34)c2)cc1 ; c1ccc(-c2cccc3c2oc2c(N(c4ccc(-c5ccc6sc7ccccc7c6c5)cc4)c4ccc5ccccc5c4)cccc23)cc1 ; c1ccc(-c2ccc(N(c3ccc(-c4ccc5sc6ccccc6c5c4)cc3)c3cccc4c3oc3c(-c5ccccc5)cccc34)c3ccccc23)cc1 ; c1ccc(-c2ccc3cc(N(c4ccc(-c5ccc6sc7ccccc7c6c5)cc4)c4cccc5c4oc4c(-c6ccccc6)cccc45)ccc3c2)cc1 ; c1ccc(-c2cccc3c2oc2c(N(c4ccc(-c5ccc6sc7ccccc7c6c5)cc4)c4cccc(-c5cccc6ccccc56)c4)cccc23)cc1]

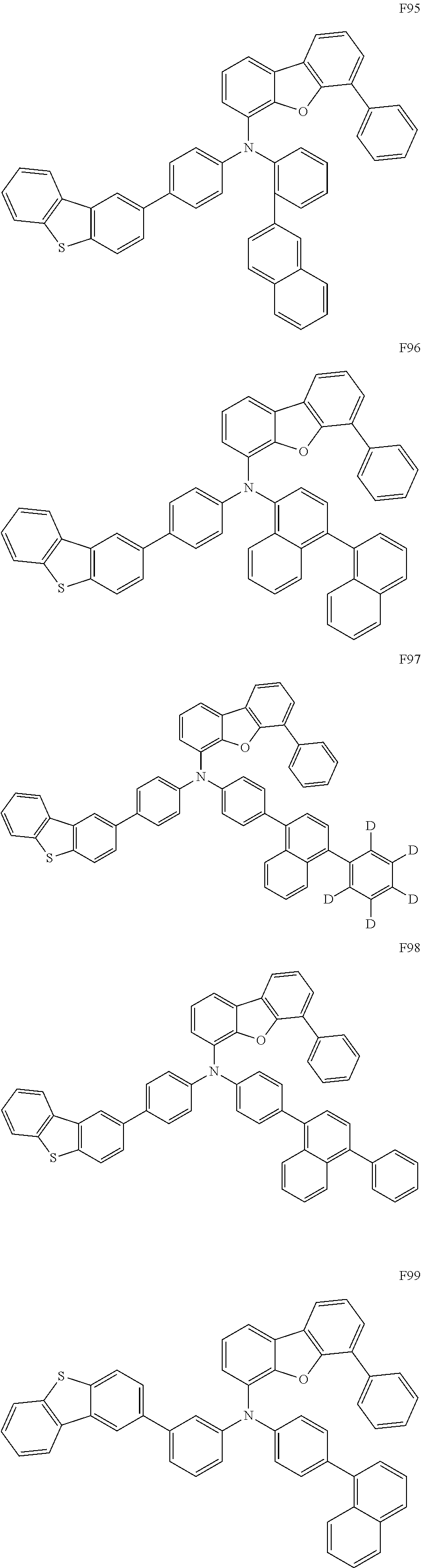

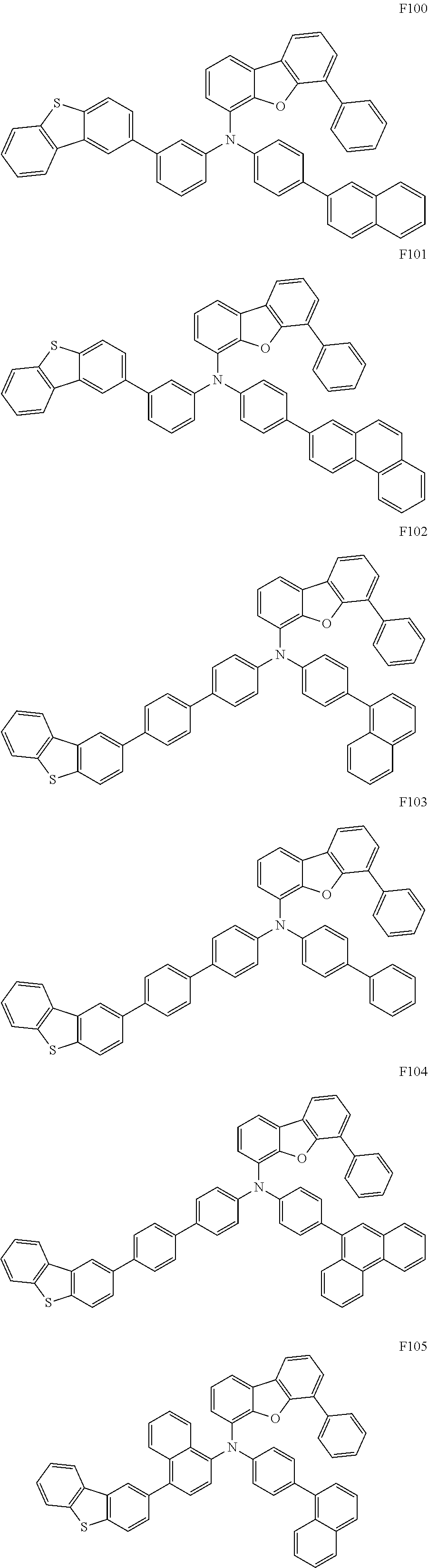

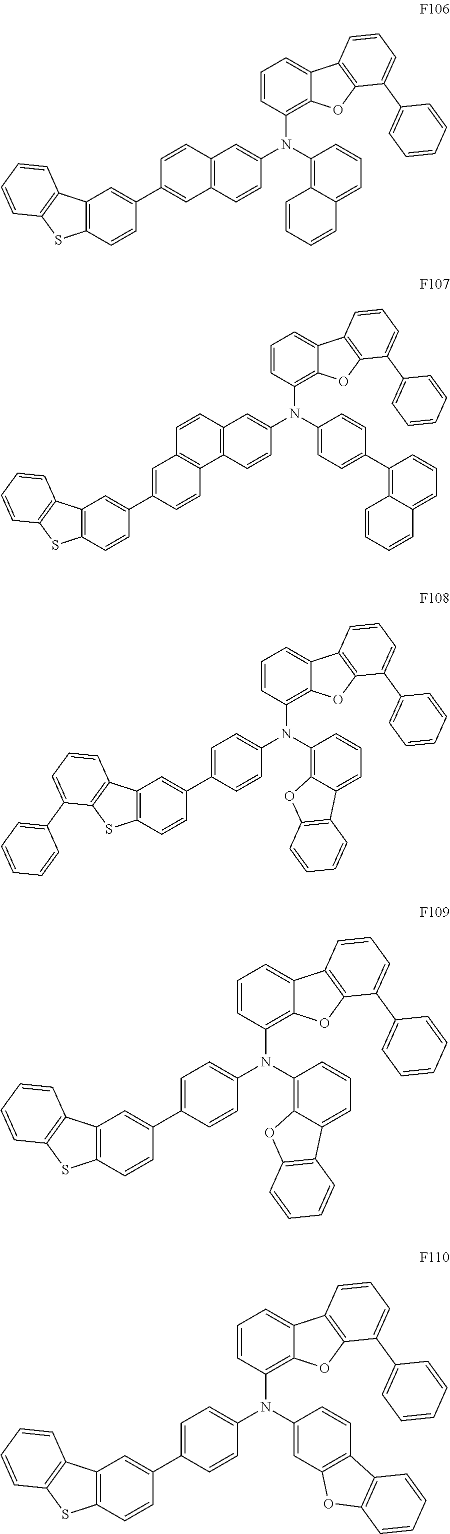 [SMILES: c1ccc(-c2cccc3c2oc2c(N(c4ccc5cc(-c6ccc7sc8ccccc8c7c6)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7ccc8sc9ccccc9c8c7)ccc65)c4)cccc23)cc1 ; c1ccc(-c2cccc3c2oc2c(N(c4ccc(-c5ccc6sc7c(-c8ccccc8)cccc7c6c5)cc4)c4cccc5c4oc4ccccc45)cccc23)cc1 ; c1ccc(-c2cccc3c2oc2c(N(c4ccc(-c5ccc6sc7ccccc7c6c5)cc4)c4cccc5c4oc4ccccc45)cccc23)cc1 ; c1ccc(-c2cccc3c2oc2c(N(c4ccc(-c5ccc6sc7ccccc7c6c5)cc4)c4ccc5c(c4)oc4ccccc45)cccc23)cc1]

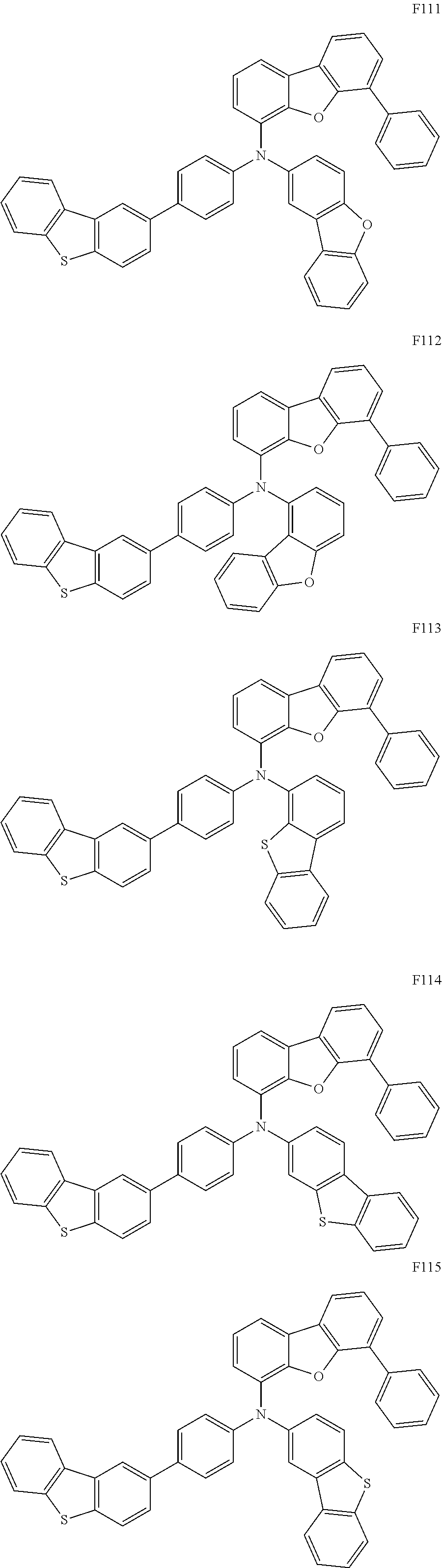

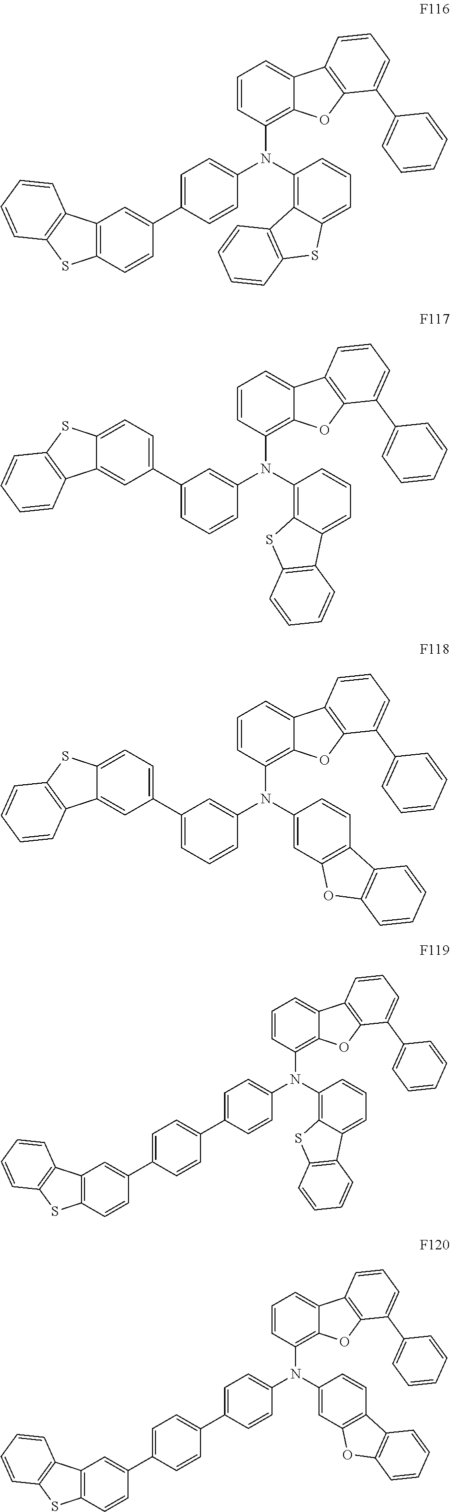 [SMILES: c1ccc(-c2cccc3c2oc2c(N(c4ccc(-c5ccc6sc7ccccc7c6c5)cc4)c4cccc5sc6ccccc6c45)cccc23)cc1 ; c1ccc(-c2cccc3c2oc2c(N(c4cccc(-c5ccc6sc7ccccc7c6c5)c4)c4cccc5c4sc4ccccc45)cccc23)cc1 ; c1ccc(-c2cccc3c2oc2c(N(c4cccc(-c5ccc6sc7ccccc7c6c5)c4)c4ccc5c(c4)oc4ccccc45)cccc23)cc1 ; c1ccc(-c2cccc3c2oc2c(N(c4ccc(-c5ccc(-c6ccc7sc8ccccc8c7c6)cc5)cc4)c4cccc5c4sc4ccccc45)cccc23)cc1 ; c1ccc(-c2cccc3c2oc2c(N(c4ccc(-c5ccc(-c6ccc7sc8ccccc8c7c6)cc5)cc4)c4ccc5c(c4)oc4ccccc45)cccc23)cc1]

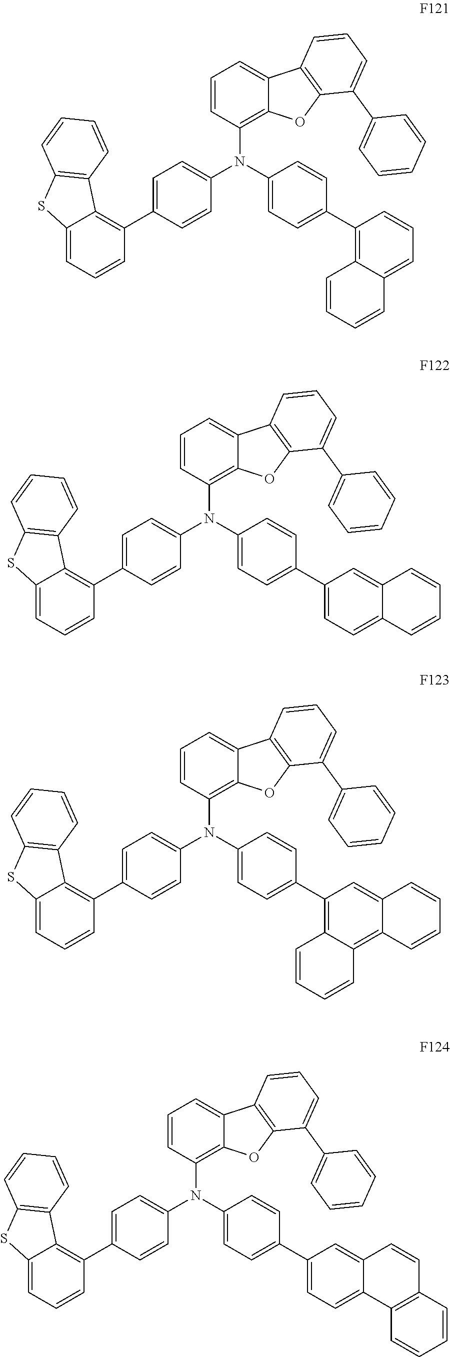 [SMILES: c1ccc(-c2cccc3c2oc2c(N(c4ccc(-c5cccc6ccccc56)cc4)c4ccc(-c5cccc6sc7ccccc7c56)cc4)cccc23)cc1 ; c1ccc(-c2cccc3c2oc2c(N(c4ccc(-c5ccc6ccccc6c5)cc4)c4ccc(-c5cccc6sc7ccccc7c56)cc4)cccc23)cc1 ; c1ccc(-c2cccc3c2oc2c(N(c4ccc(-c5cc6ccccc6c6ccccc56)cc4)c4ccc(-c5cccc6sc7ccccc7c56)cc4)cccc23)cc1 ; c1ccc(-c2cccc3c2oc2c(N(c4ccc(-c5ccc6c(ccc7ccccc76)c5)cc4)c4ccc(-c5cccc6sc7ccccc7c56)cc4)cccc23)cc1]

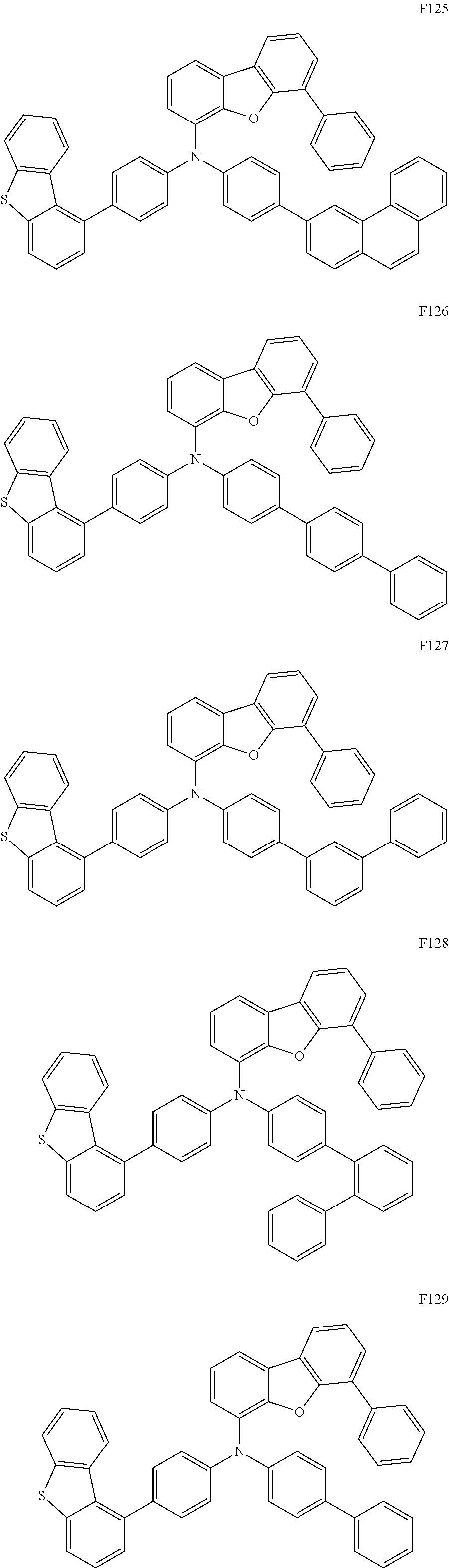 [SMILES: c1ccc(-c2cccc3c2oc2c(N(c4ccc(-c5ccc6ccc7ccccc7c6c5)cc4)c4ccc(-c5cccc6sc7ccccc7c56)cc4)cccc23)cc1 ; c1ccc(-c2ccc(-c3ccc(N(c4ccc(-c5cccc6sc7ccccc7c56)cc4)c4cccc5c4oc4c(-c6ccccc6)cccc45)cc3)cc2)cc1 ; c1ccc(-c2cccc(-c3ccc(N(c4ccc(-c5cccc6sc7ccccc7c56)cc4)c4cccc5c4oc4c(-c6ccccc6)cccc45)cc3)c2)cc1 ; c1ccc(-c2ccccc2-c2ccc(N(c3ccc(-c4cccc5sc6ccccc6c45)cc3)c3cccc4c3oc3c(-c5ccccc5)cccc34)cc2)cc1 ; c1ccc(-c2ccc(N(c3ccc(-c4cccc5sc6ccccc6c45)cc3)c3cccc4c3oc3c(-c5ccccc5)cccc34)cc2)cc1]

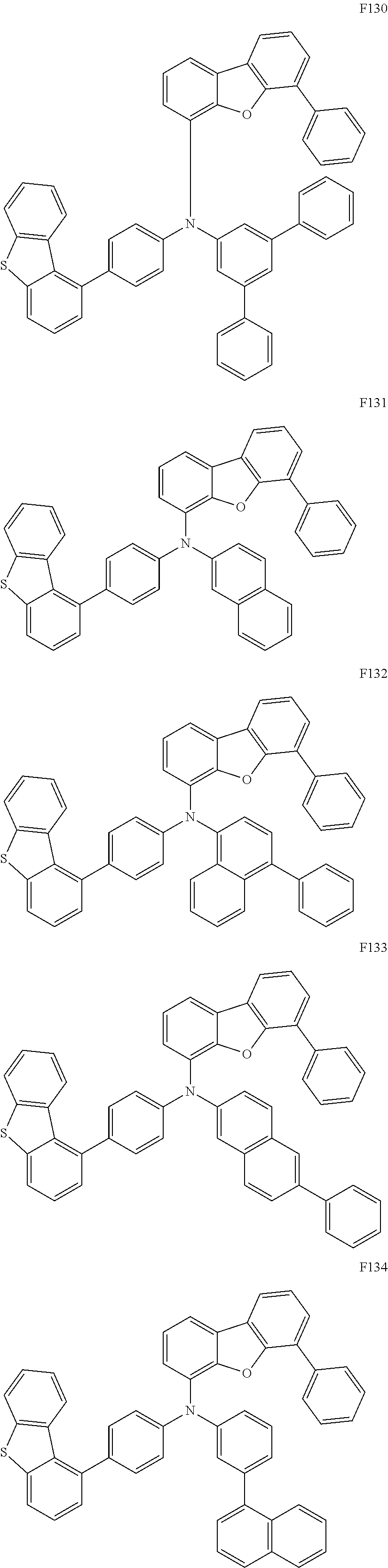 [SMILES: c1ccc(-c2cc(-c3ccccc3)cc(N(c3ccc(-c4cccc5sc6ccccc6c45)cc3)c3cccc4c3oc3c(-c5ccccc5)cccc34)c2)cc1 ; c1ccc(-c2cccc3c2oc2c(N(c4ccc(-c5cccc6sc7ccccc7c56)cc4)c4ccc5ccccc5c4)cccc23)cc1 ; c1ccc(-c2ccc(N(c3ccc(-c4cccc5sc6ccccc6c45)cc3)c3cccc4c3oc3c(-c5ccccc5)cccc34)c3ccccc23)cc1 ; c1ccc(-c2ccc3cc(N(c4ccc(-c5cccc6sc7ccccc7c56)cc4)c4cccc5c4oc4c(-c6ccccc6)cccc45)ccc3c2)cc1 ; c1ccc(-c2cccc3c2oc2c(N(c4ccc(-c5cccc6sc7ccccc7c56)cc4)c4cccc(-c5cccc6ccccc56)c4)cccc23)cc1]

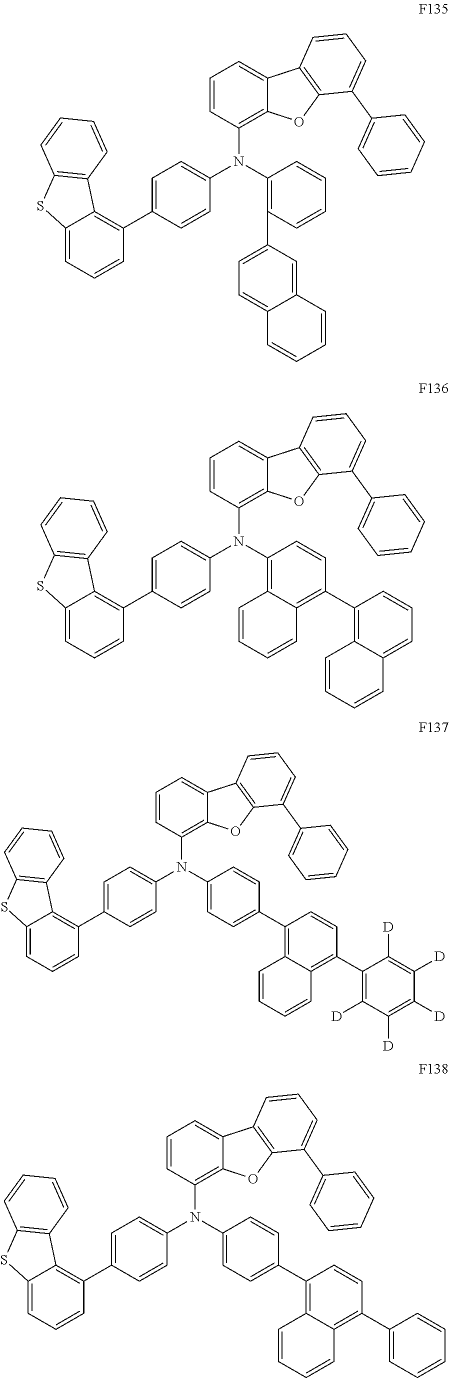 [SMILES: c1ccc(-c2cccc3c2oc2c(N(c4ccc(-c5cccc6sc7ccccc7c56)cc4)c4ccccc4-c4ccc5ccccc5c4)cccc23)cc1 ; c1ccc(-c2cccc3c2oc2c(N(c4ccc(-c5cccc6sc7ccccc7c56)cc4)c4ccc(-c5cccc6ccccc56)c5ccccc45)cccc23)cc1 ; [2H]c1c([2H])c([2H])c(-c2ccc(-c3ccc(N(c4ccc(-c5cccc6sc7ccccc7c56)cc4)c4cccc5c4oc4c(-c6ccccc6)cccc45)cc3)c3ccccc23)c([2H])c1[2H] ; c1ccc(-c2ccc(-c3ccc(N(c4ccc(-c5cccc6sc7ccccc7c56)cc4)c4cccc5c4oc4c(-c6ccccc6)cccc45)cc3)c3ccccc23)cc1]

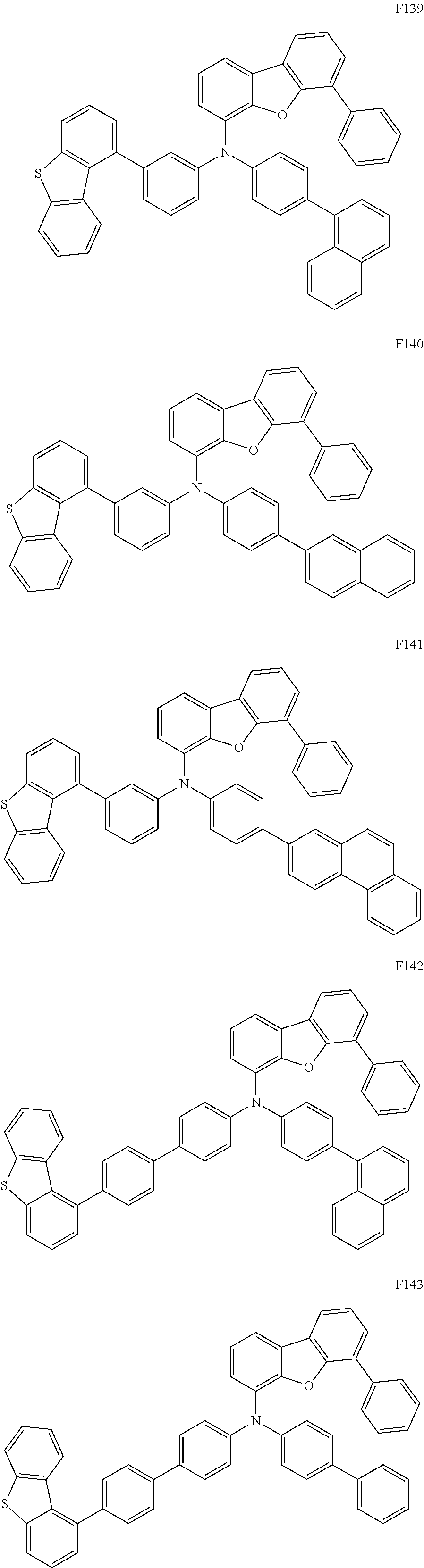 [SMILES: c1ccc(-c2cccc3c2oc2c(N(c4ccc(-c5cccc6ccccc56)cc4)c4cccc(-c5cccc6sc7ccccc7c56)c4)cccc23)cc1 ; c1ccc(-c2cccc3c2oc2c(N(c4ccc(-c5ccc6ccccc6c5)cc4)c4cccc(-c5cccc6sc7ccccc7c56)c4)cccc23)cc1 ; c1ccc(-c2cccc3c2oc2c(N(c4ccc(-c5ccc6c(ccc7ccccc76)c5)cc4)c4cccc(-c5cccc6sc7ccccc7c56)c4)cccc23)cc1 ; c1ccc(-c2cccc3c2oc2c(N(c4ccc(-c5ccc(-c6cccc7sc8ccccc8c67)cc5)cc4)c4ccc(-c5cccc6ccccc56)cc4)cccc23)cc1 ; c1ccc(-c2ccc(N(c3ccc(-c4ccc(-c5cccc6sc7ccccc7c56)cc4)cc3)c3cccc4c3oc3c(-c5ccccc5)cccc34)cc2)cc1]

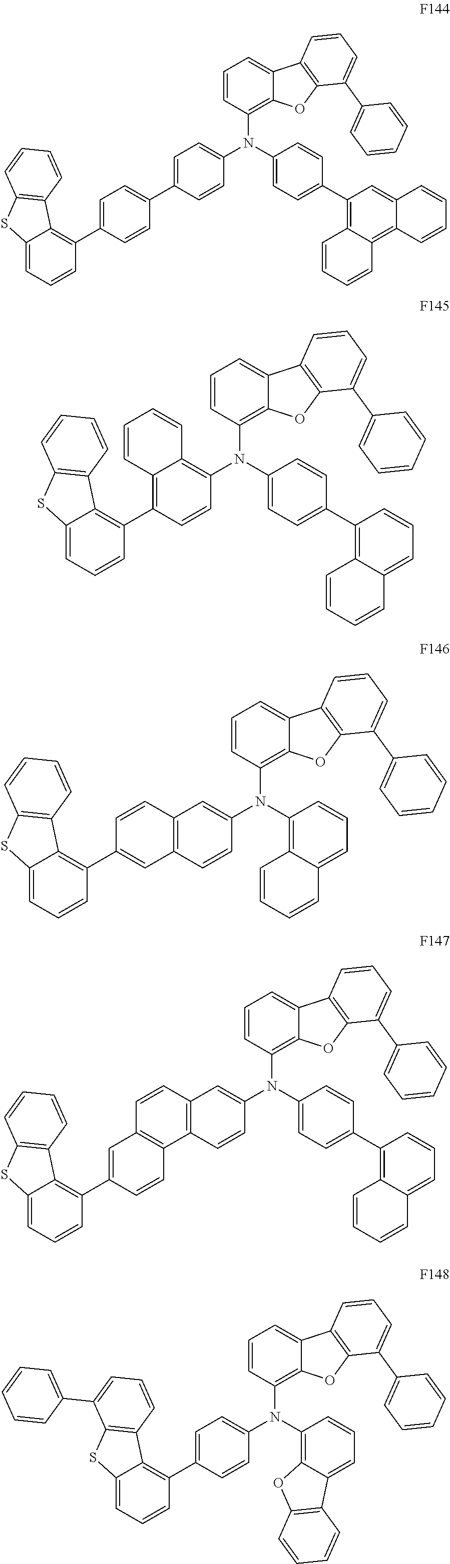 [SMILES: c1ccc(-c2cccc3c2oc2c(N(c4ccc(-c5ccc(-c6cccc7sc8ccccc8c67)cc5)cc4)c4ccc(-c5cc6ccccc6c6ccccc56)cc4)cccc23)cc1 ; c1ccc(-c2cccc3c2oc2c(N(c4ccc(-c5cccc6ccccc56)cc4)c4ccc(-c5cccc6sc7ccccc7c56)c5ccccc45)cccc23)cc1 ; c1ccc(-c2cccc3c2oc2c(N(c4ccc5cc(-c6cccc7sc8ccccc8c67)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7cccc8sc9ccccc9c78)ccc65)c4)cccc23)cc1 ; c1ccc(-c2cccc3c2oc2c(N(c4ccc(-c5cccc6sc7c(-c8ccccc8)cccc7c56)cc4)c4cccc5c4oc4ccccc45)cccc23)cc1]

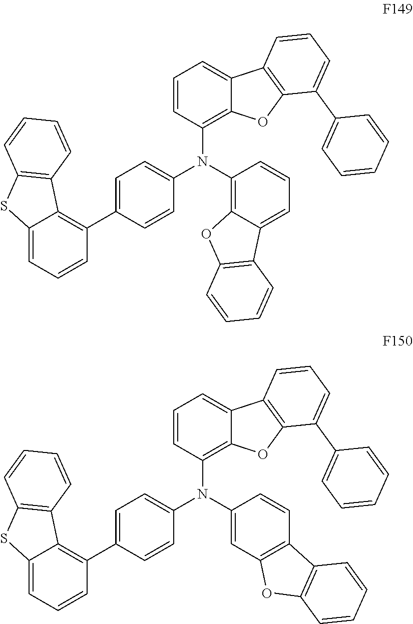 [SMILES: c1ccc(-c2cccc3c2oc2c(N(c4ccc(-c5cccc6sc7ccccc7c56)cc4)c4cccc5c4oc4ccccc45)cccc23)cc1 ; c1ccc(-c2cccc3c2oc2c(N(c4ccc(-c5cccc6sc7ccccc7c56)cc4)c4ccc5c(c4)oc4ccccc45)cccc23)cc1]

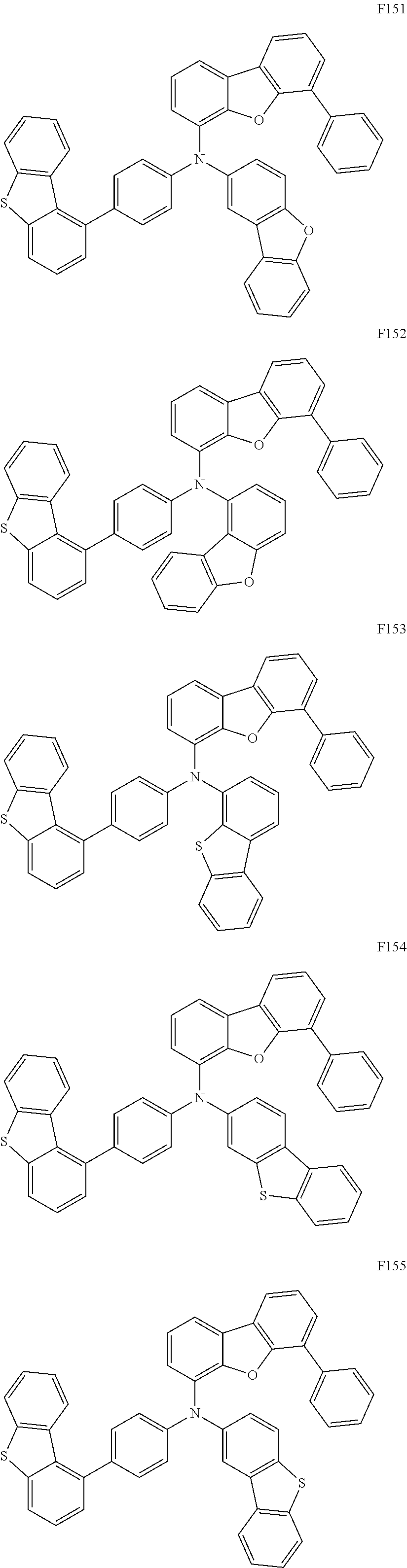

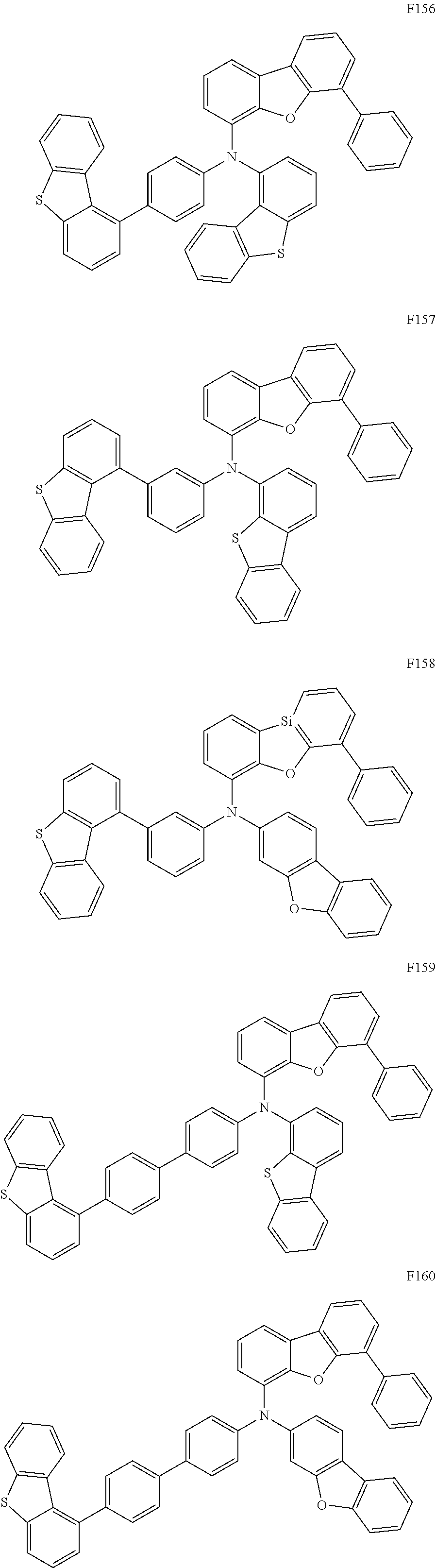 [SMILES: c1ccc(-c2cccc3c2oc2c(N(c4ccc(-c5cccc6sc7ccccc7c56)cc4)c4cccc5sc6ccccc6c45)cccc23)cc1 ; c1ccc(-c2cccc3c2oc2c(N(c4cccc(-c5cccc6sc7ccccc7c56)c4)c4cccc5c4sc4ccccc45)cccc23)cc1 ; c1ccc(-c2ccc[si]3c2oc2c(N(c4cccc(-c5cccc6sc7ccccc7c56)c4)c4ccc5c(c4)oc4ccccc45)cccc23)cc1 ; c1ccc(-c2cccc3c2oc2c(N(c4ccc(-c5ccc(-c6cccc7sc8ccccc8c67)cc5)cc4)c4cccc5c4sc4ccccc45)cccc23)cc1 ; c1ccc(-c2cccc3c2oc2c(N(c4ccc(-c5ccc(-c6cccc7sc8ccccc8c67)cc5)cc4)c4ccc5c(c4)oc4ccccc45)cccc23)cc1]

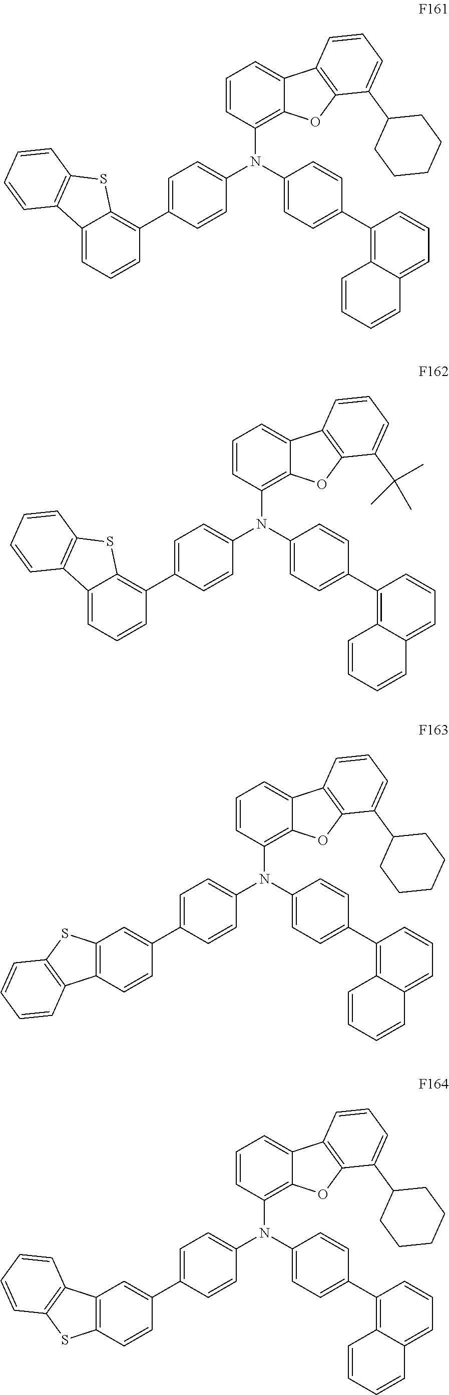 [SMILES: c1ccc2c(-c3ccc(N(c4ccc(-c5cccc6c5sc5ccccc56)cc4)c4cccc5c4oc4c(C6CCCCC6)cccc45)cc3)cccc2c1 ; CC(C)(C)c1cccc2c1oc1c(N(c3ccc(-c4cccc5ccccc45)cc3)c3ccc(-c4cccc5c4sc4ccccc45)cc3)cccc12 ; c1ccc2c(-c3ccc(N(c4ccc(-c5ccc6c(c5)sc5ccccc56)cc4)c4cccc5c4oc4c(C6CCCCC6)cccc45)cc3)cccc2c1 ; c1ccc2c(-c3ccc(N(c4ccc(-c5ccc6sc7ccccc7c6c5)cc4)c4cccc5c4oc4c(C6CCCCC6)cccc45)cc3)cccc2c1]

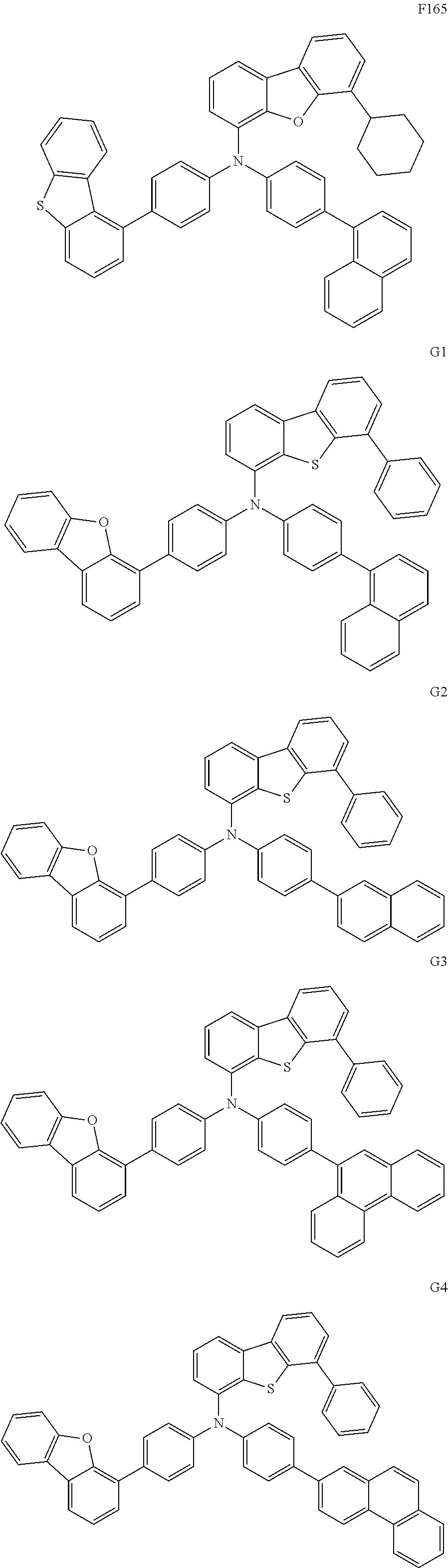 [SMILES: c1ccc2c(-c3ccc(N(c4ccc(-c5cccc6sc7ccccc7c56)cc4)c4cccc5c4oc4c(C6CCCCC6)cccc45)cc3)cccc2c1 ; c1ccc(-c2cccc3c2sc2c(N(c4ccc(-c5cccc6ccccc56)cc4)c4ccc(-c5cccc6c5oc5ccccc56)cc4)cccc23)cc1 ; c1ccc(-c2cccc3c2sc2c(N(c4ccc(-c5ccc6ccccc6c5)cc4)c4ccc(-c5cccc6c5oc5ccccc56)cc4)cccc23)cc1 ; c1ccc(-c2cccc3c2sc2c(N(c4ccc(-c5cc6ccccc6c6ccccc56)cc4)c4ccc(-c5cccc6c5oc5ccccc56)cc4)cccc23)cc1 ; c1ccc(-c2cccc3c2sc2c(N(c4ccc(-c5ccc6c(ccc7ccccc76)c5)cc4)c4ccc(-c5cccc6c5oc5ccccc56)cc4)cccc23)cc1]

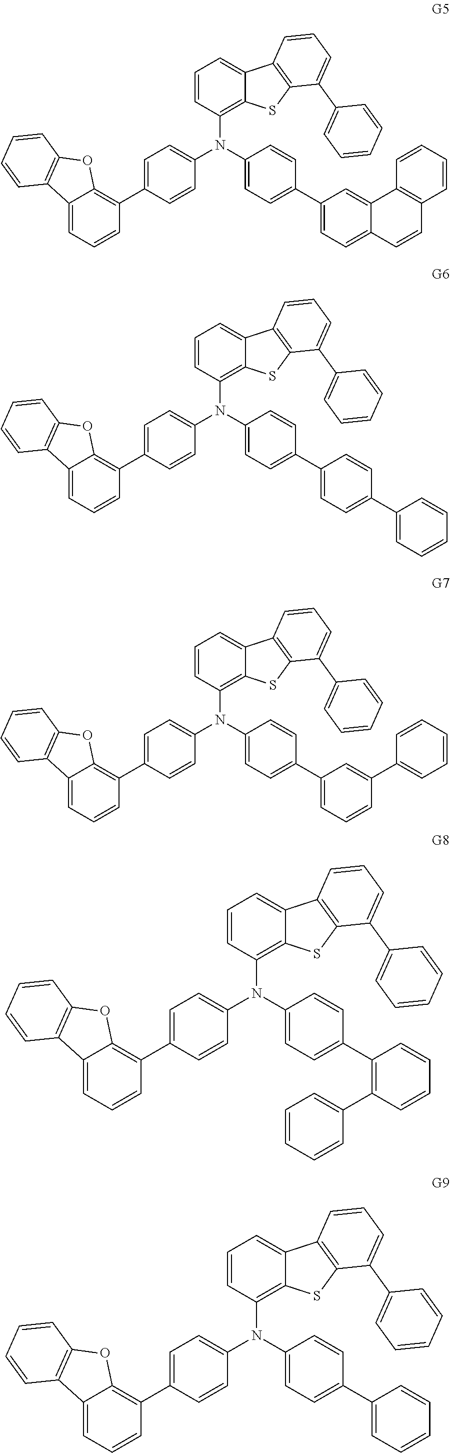 [SMILES: c1ccc(-c2cccc3c2sc2c(N(c4ccc(-c5ccc6ccc7ccccc7c6c5)cc4)c4ccc(-c5cccc6c5oc5ccccc56)cc4)cccc23)cc1 ; c1ccc(-c2ccc(-c3ccc(N(c4ccc(-c5cccc6c5oc5ccccc56)cc4)c4cccc5c4sc4c(-c6ccccc6)cccc45)cc3)cc2)cc1 ; c1ccc(-c2cccc(-c3ccc(N(c4ccc(-c5cccc6c5oc5ccccc56)cc4)c4cccc5c4sc4c(-c6ccccc6)cccc45)cc3)c2)cc1 ; c1ccc(-c2ccccc2-c2ccc(N(c3ccc(-c4cccc5c4oc4ccccc45)cc3)c3cccc4c3sc3c(-c5ccccc5)cccc34)cc2)cc1 ; c1ccc(-c2ccc(N(c3ccc(-c4cccc5c4oc4ccccc45)cc3)c3cccc4c3sc3c(-c5ccccc5)cccc34)cc2)cc1]

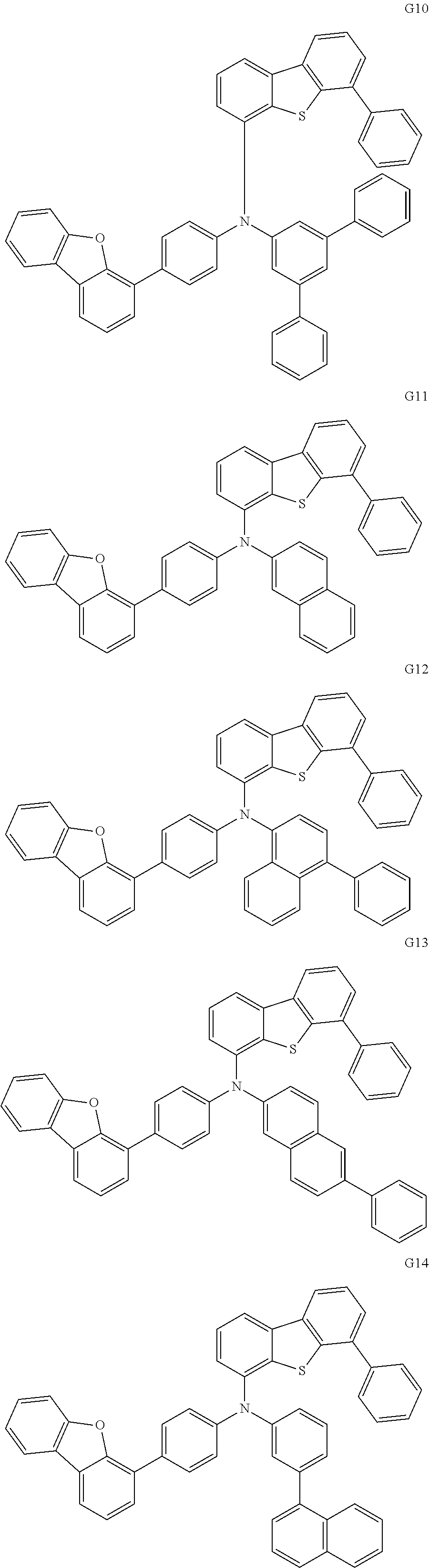 [SMILES: c1ccc(-c2cc(-c3ccccc3)cc(N(c3ccc(-c4cccc5c4oc4ccccc45)cc3)c3cccc4c3sc3c(-c5ccccc5)cccc34)c2)cc1 ; c1ccc(-c2cccc3c2sc2c(N(c4ccc(-c5cccc6c5oc5ccccc56)cc4)c4ccc5ccccc5c4)cccc23)cc1 ; c1ccc(-c2ccc(N(c3ccc(-c4cccc5c4oc4ccccc45)cc3)c3cccc4c3sc3c(-c5ccccc5)cccc34)c3ccccc23)cc1 ; c1ccc(-c2ccc3cc(N(c4ccc(-c5cccc6c5oc5ccccc56)cc4)c4cccc5c4sc4c(-c6ccccc6)cccc45)ccc3c2)cc1 ; c1ccc(-c2cccc3c2sc2c(N(c4ccc(-c5cccc6c5oc5ccccc56)cc4)c4cccc(-c5cccc6ccccc56)c4)cccc23)cc1]

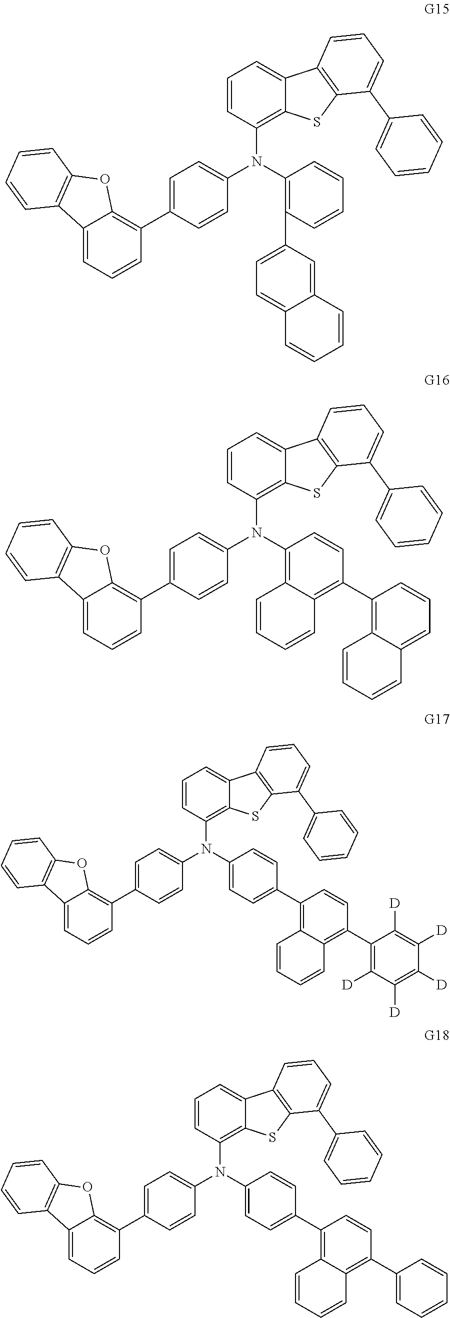 [SMILES: c1ccc(-c2cccc3c2sc2c(N(c4ccc(-c5cccc6c5oc5ccccc56)cc4)c4ccccc4-c4ccc5ccccc5c4)cccc23)cc1 ; c1ccc(-c2cccc3c2sc2c(N(c4ccc(-c5cccc6c5oc5ccccc56)cc4)c4ccc(-c5cccc6ccccc56)c5ccccc45)cccc23)cc1 ; [2H]c1c([2H])c([2H])c(-c2ccc(-c3ccc(N(c4ccc(-c5cccc6c5oc5ccccc56)cc4)c4cccc5c4sc4c(-c6ccccc6)cccc45)cc3)c3ccccc23)c([2H])c1[2H] ; c1ccc(-c2ccc(-c3ccc(N(c4ccc(-c5cccc6c5oc5ccccc56)cc4)c4cccc5c4sc4c(-c6ccccc6)cccc45)cc3)c3ccccc23)cc1]

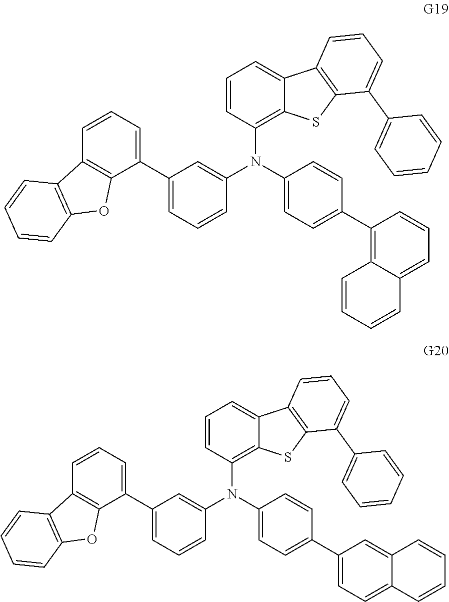 [SMILES: c1ccc(-c2cccc3c2sc2c(N(c4ccc(-c5cccc6ccccc56)cc4)c4cccc(-c5cccc6c5oc5ccccc56)c4)cccc23)cc1 ; c1ccc(-c2cccc3c2sc2c(N(c4ccc(-c5ccc6ccccc6c5)cc4)c4cccc(-c5cccc6c5oc5ccccc56)c4)cccc23)cc1]

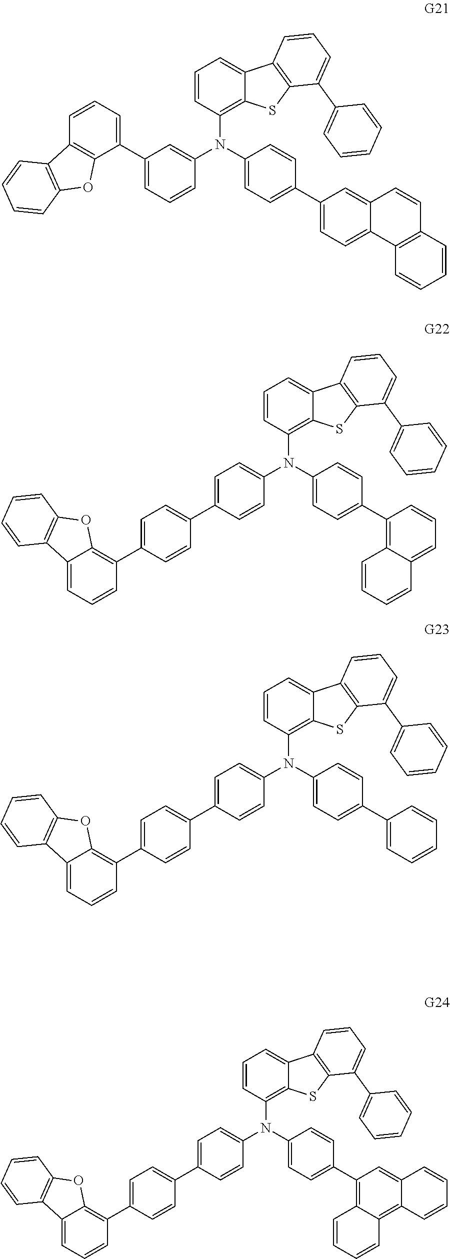 [SMILES: c1ccc(-c2cccc3c2sc2c(N(c4ccc(-c5ccc6c(ccc7ccccc76)c5)cc4)c4cccc(-c5cccc6c5oc5ccccc56)c4)cccc23)cc1 ; c1ccc(-c2cccc3c2sc2c(N(c4ccc(-c5ccc(-c6cccc7c6oc6ccccc67)cc5)cc4)c4ccc(-c5cccc6ccccc56)cc4)cccc23)cc1 ; c1ccc(-c2ccc(N(c3ccc(-c4ccc(-c5cccc6c5oc5ccccc56)cc4)cc3)c3cccc4c3sc3c(-c5ccccc5)cccc34)cc2)cc1 ; c1ccc(-c2cccc3c2sc2c(N(c4ccc(-c5ccc(-c6cccc7c6oc6ccccc67)cc5)cc4)c4ccc(-c5cc6ccccc6c6ccccc56)cc4)cccc23)cc1]

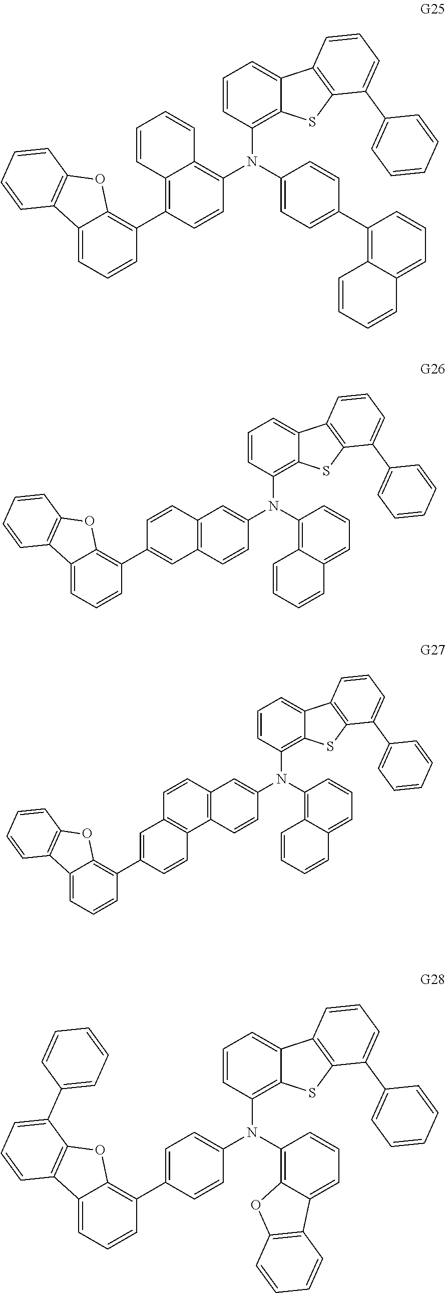 [SMILES: c1ccc(-c2cccc3c2sc2c(N(c4ccc(-c5cccc6ccccc56)cc4)c4ccc(-c5cccc6c5oc5ccccc56)c5ccccc45)cccc23)cc1 ; c1ccc(-c2cccc3c2sc2c(N(c4ccc5cc(-c6cccc7c6oc6ccccc67)ccc5c4)c4cccc5ccccc45)cccc23)cc1 ; c1ccc(-c2cccc3c2sc2c(N(c4ccc5c(ccc6cc(-c7cccc8c7oc7ccccc78)ccc65)c4)c4cccc5ccccc45)cccc23)cc1 ; c1ccc(-c2cccc3c2oc2c(-c4ccc(N(c5cccc6c5oc5ccccc56)c5cccc6c5sc5c(-c7ccccc7)cccc56)cc4)cccc23)cc1]

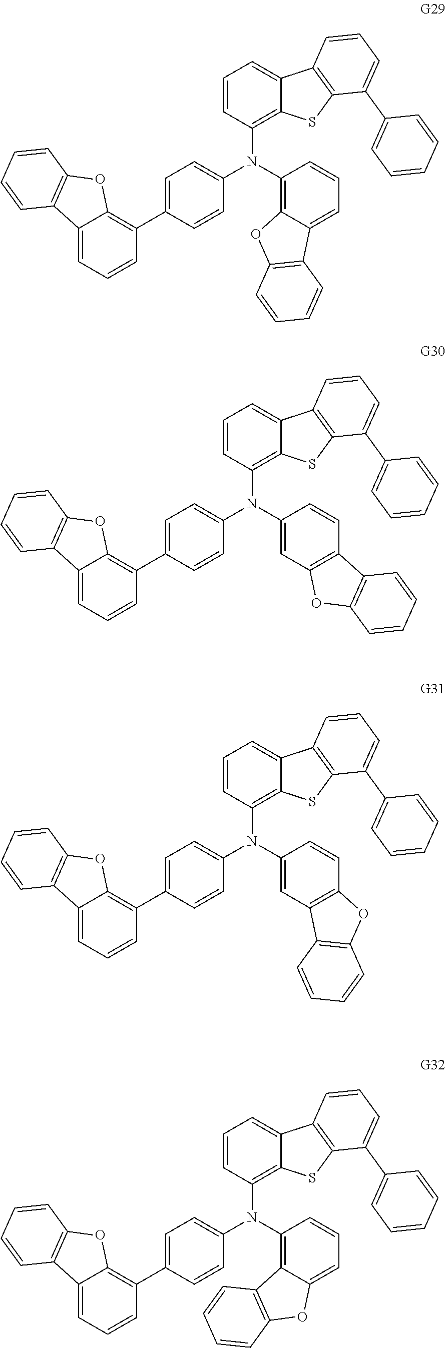 [SMILES: c1ccc(-c2cccc3c2sc2c(N(c4ccc(-c5cccc6c5oc5ccccc56)cc4)c4cccc5c4oc4ccccc45)cccc23)cc1 ; c1ccc(-c2cccc3c2sc2c(N(c4ccc(-c5cccc6c5oc5ccccc56)cc4)c4ccc5c(c4)oc4ccccc45)cccc23)cc1 ; c1ccc(-c2cccc3c2sc2c(N(c4ccc(-c5cccc6c5oc5ccccc56)cc4)c4ccc5oc6ccccc6c5c4)cccc23)cc1 ; c1ccc(-c2cccc3c2sc2c(N(c4ccc(-c5cccc6c5oc5ccccc56)cc4)c4cccc5oc6ccccc6c45)cccc23)cc1]

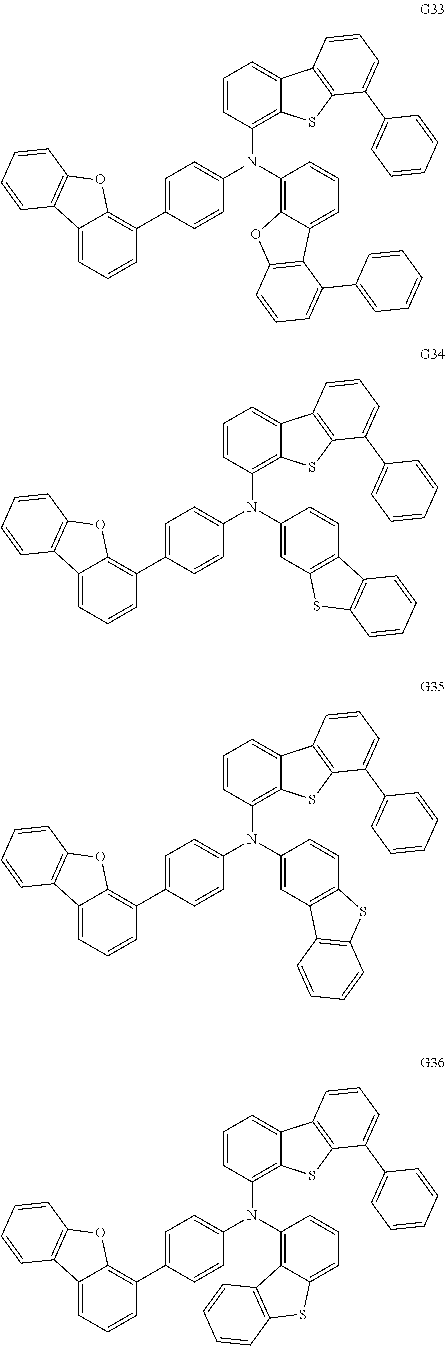 [SMILES: c1ccc(-c2cccc3c2sc2c(N(c4ccc(-c5cccc6c5oc5ccccc56)cc4)c4cccc5c4oc4cccc(-c6ccccc6)c45)cccc23)cc1 ; c1ccc(-c2cccc3c2sc2c(N(c4ccc(-c5cccc6c5oc5ccccc56)cc4)c4ccc5c(c4)sc4ccccc45)cccc23)cc1 ; c1ccc(-c2cccc3c2sc2c(N(c4ccc(-c5cccc6c5oc5ccccc56)cc4)c4ccc5sc6ccccc6c5c4)cccc23)cc1 ; c1ccc(-c2cccc3c2sc2c(N(c4ccc(-c5cccc6c5oc5ccccc56)cc4)c4cccc5sc6ccccc6c45)cccc23)cc1]

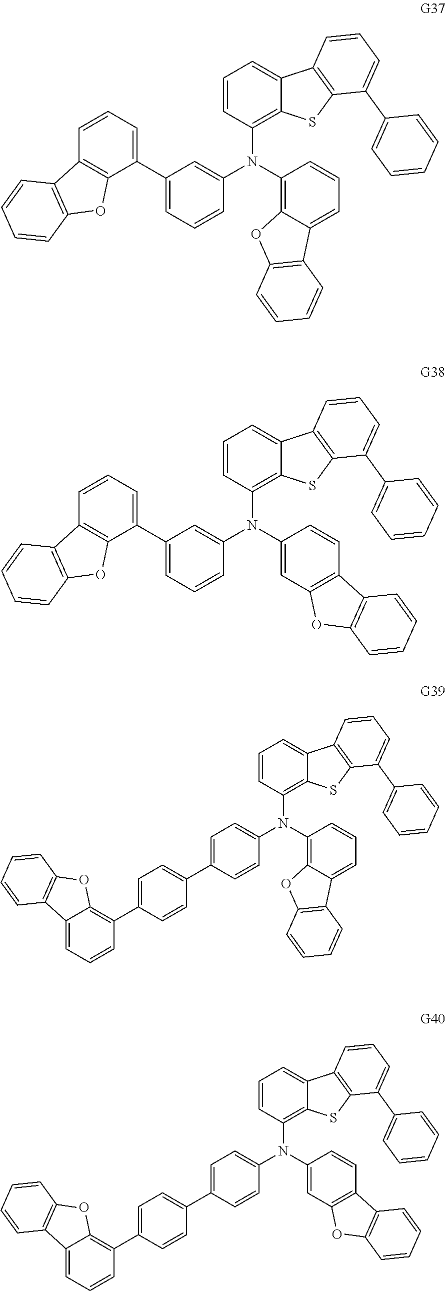 [SMILES: c1ccc(-c2cccc3c2sc2c(N(c4cccc(-c5cccc6c5oc5ccccc56)c4)c4cccc5c4oc4ccccc45)cccc23)cc1 ; c1ccc(-c2cccc3c2sc2c(N(c4cccc(-c5cccc6c5oc5ccccc56)c4)c4ccc5c(c4)oc4ccccc45)cccc23)cc1 ; c1ccc(-c2cccc3c2sc2c(N(c4ccc(-c5ccc(-c6cccc7c6oc6ccccc67)cc5)cc4)c4cccc5c4oc4ccccc45)cccc23)cc1 ; c1ccc(-c2cccc3c2sc2c(N(c4ccc(-c5ccc(-c6cccc7c6oc6ccccc67)cc5)cc4)c4ccc5c(c4)oc4ccccc45)cccc23)cc1]

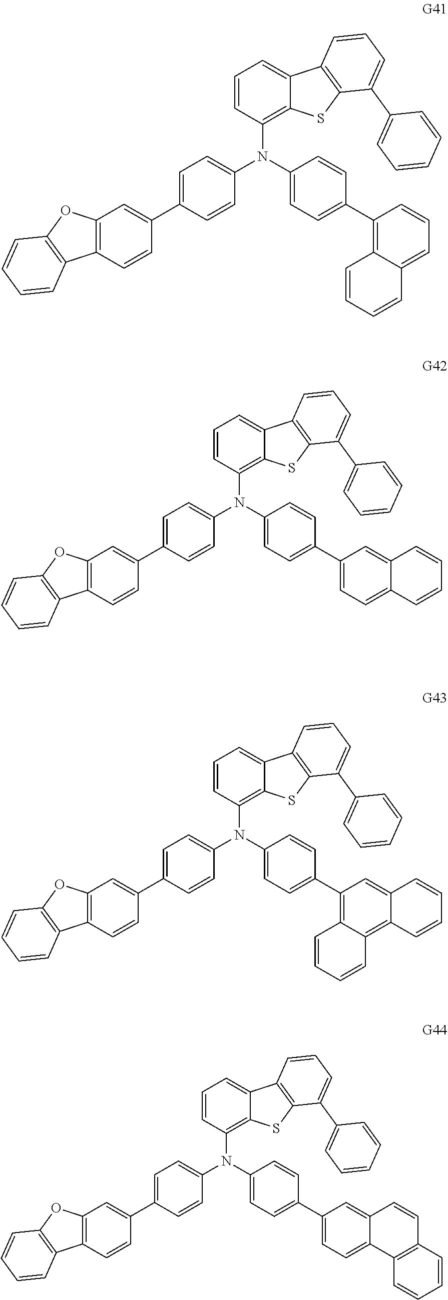 [SMILES: c1ccc(-c2cccc3c2sc2c(N(c4ccc(-c5ccc6c(c5)oc5ccccc56)cc4)c4ccc(-c5cccc6ccccc56)cc4)cccc23)cc1 ; c1ccc(-c2cccc3c2sc2c(N(c4ccc(-c5ccc6ccccc6c5)cc4)c4ccc(-c5ccc6c(c5)oc5ccccc56)cc4)cccc23)cc1 ; c1ccc(-c2cccc3c2sc2c(N(c4ccc(-c5ccc6c(c5)oc5ccccc56)cc4)c4ccc(-c5cc6ccccc6c6ccccc56)cc4)cccc23)cc1 ; c1ccc(-c2cccc3c2sc2c(N(c4ccc(-c5ccc6c(ccc7ccccc76)c5)cc4)c4ccc(-c5ccc6c(c5)oc5ccccc56)cc4)cccc23)cc1]

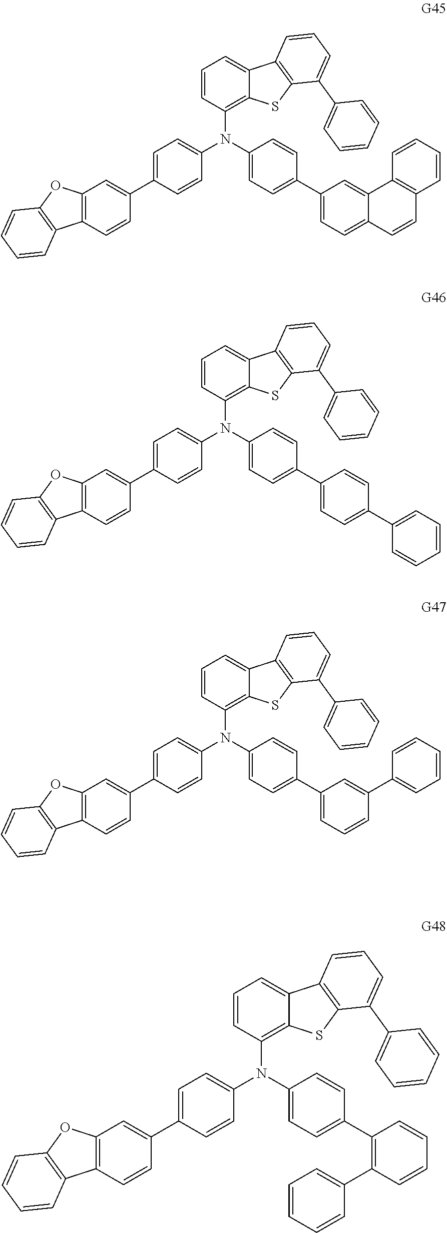 [SMILES: c1ccc(-c2cccc3c2sc2c(N(c4ccc(-c5ccc6c(c5)oc5ccccc56)cc4)c4ccc(-c5ccc6ccc7ccccc7c6c5)cc4)cccc23)cc1 ; c1ccc(-c2ccc(-c3ccc(N(c4ccc(-c5ccc6c(c5)oc5ccccc56)cc4)c4cccc5c4sc4c(-c6ccccc6)cccc45)cc3)cc2)cc1 ; c1ccc(-c2cccc(-c3ccc(N(c4ccc(-c5ccc6c(c5)oc5ccccc56)cc4)c4cccc5c4sc4c(-c6ccccc6)cccc45)cc3)c2)cc1 ; c1ccc(-c2ccccc2-c2ccc(N(c3ccc(-c4ccc5c(c4)oc4ccccc45)cc3)c3cccc4c3sc3c(-c5ccccc5)cccc34)cc2)cc1]

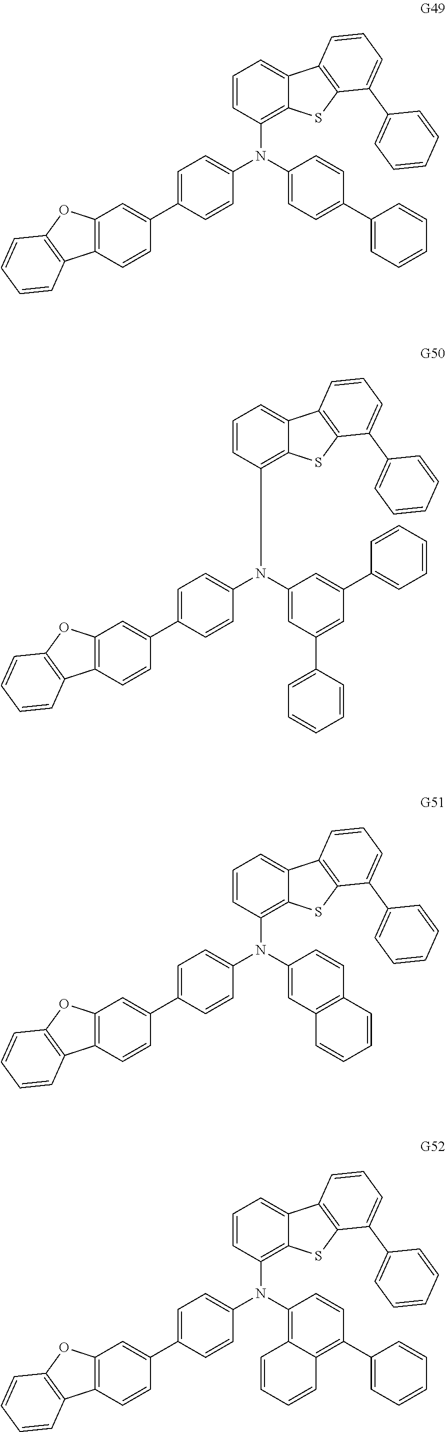 [SMILES: c1ccc(-c2ccc(N(c3ccc(-c4ccc5c(c4)oc4ccccc45)cc3)c3cccc4c3sc3c(-c5ccccc5)cccc34)cc2)cc1 ; c1ccc(-c2cc(-c3ccccc3)cc(N(c3ccc(-c4ccc5c(c4)oc4ccccc45)cc3)c3cccc4c3sc3c(-c5ccccc5)cccc34)c2)cc1 ; c1ccc(-c2cccc3c2sc2c(N(c4ccc(-c5ccc6c(c5)oc5ccccc56)cc4)c4ccc5ccccc5c4)cccc23)cc1 ; c1ccc(-c2ccc(N(c3ccc(-c4ccc5c(c4)oc4ccccc45)cc3)c3cccc4c3sc3c(-c5ccccc5)cccc34)c3ccccc23)cc1]

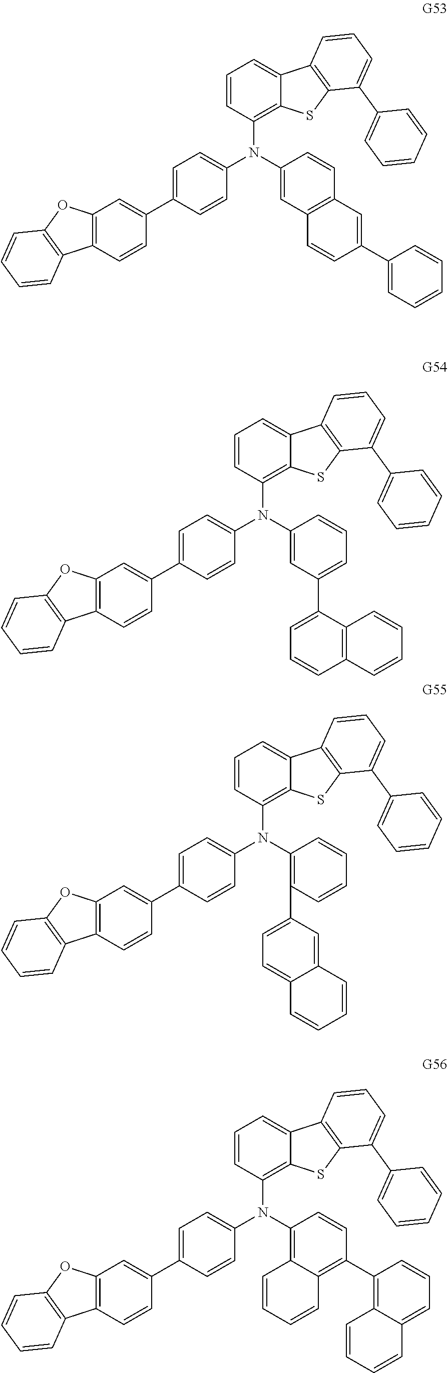

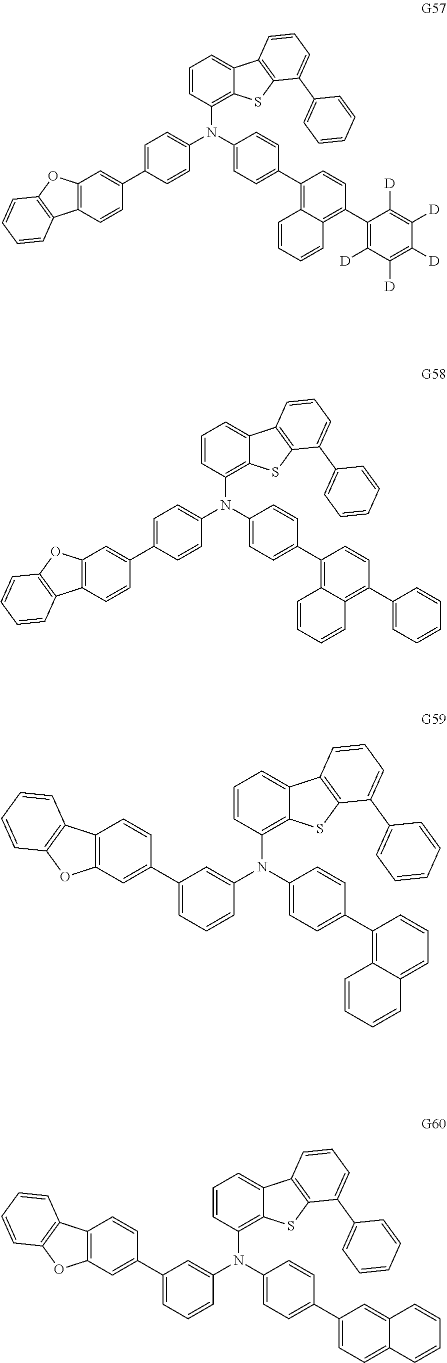 [SMILES: [2H]c1c([2H])c([2H])c(-c2ccc(-c3ccc(N(c4ccc(-c5ccc6c(c5)oc5ccccc56)cc4)c4cccc5c4sc4c(-c6ccccc6)cccc45)cc3)c3ccccc23)c([2H])c1[2H] ; c1ccc(-c2ccc(-c3ccc(N(c4ccc(-c5ccc6c(c5)oc5ccccc56)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4cccc(-c5ccc6c(c5)oc5ccccc56)c4)cccc23)cc1 ; c1ccc(-c2cccc3c2sc2c(N(c4ccc(-c5ccc6ccccc6c5)cc4)c4cccc(-c5ccc6c(c5)oc5ccccc56)c4)cccc23)cc1]

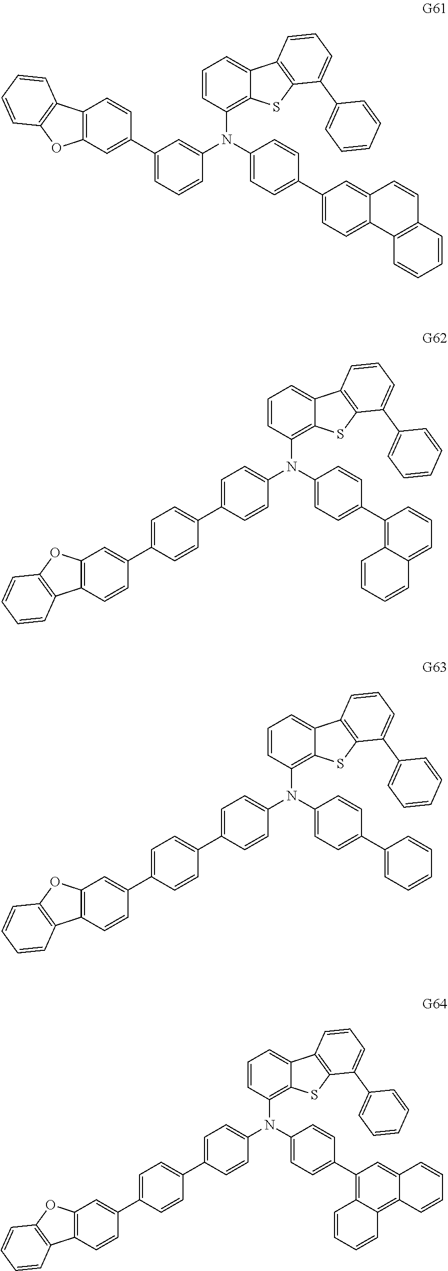 [SMILES: c1ccc(-c2cccc3c2sc2c(N(c4ccc(-c5ccc6c(ccc7ccccc76)c5)cc4)c4cccc(-c5ccc6c(c5)oc5ccccc56)c4)cccc23)cc1 ; c1ccc(-c2cccc3c2sc2c(N(c4ccc(-c5ccc(-c6ccc7c(c6)oc6ccccc67)cc5)cc4)c4ccc(-c5cccc6ccccc56)cc4)cccc23)cc1 ; c1ccc(-c2ccc(N(c3ccc(-c4ccc(-c5ccc6c(c5)oc5ccccc56)cc4)cc3)c3cccc4c3sc3c(-c5ccccc5)cccc34)cc2)cc1 ; c1ccc(-c2cccc3c2sc2c(N(c4ccc(-c5ccc(-c6ccc7c(c6)oc6ccccc67)cc5)cc4)c4ccc(-c5cc6ccccc6c6ccccc56)cc4)cccc23)cc1]

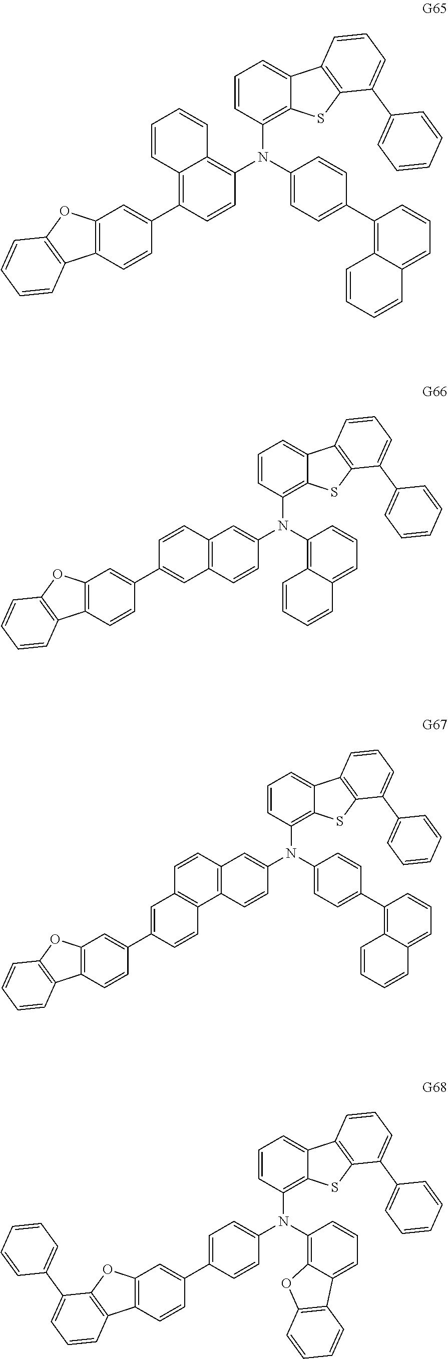 [SMILES: c1ccc(-c2cccc3c2sc2c(N(c4ccc(-c5cccc6ccccc56)cc4)c4ccc(-c5ccc6c(c5)oc5ccccc56)c5ccccc45)cccc23)cc1 ; c1ccc(-c2cccc3c2sc2c(N(c4ccc5cc(-c6ccc7c(c6)oc6ccccc67)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7ccc8c(c7)oc7ccccc78)ccc65)c4)cccc23)cc1 ; c1ccc(-c2cccc3c2oc2cc(-c4ccc(N(c5cccc6c5oc5ccccc56)c5cccc6c5sc5c(-c7ccccc7)cccc56)cc4)ccc23)cc1]

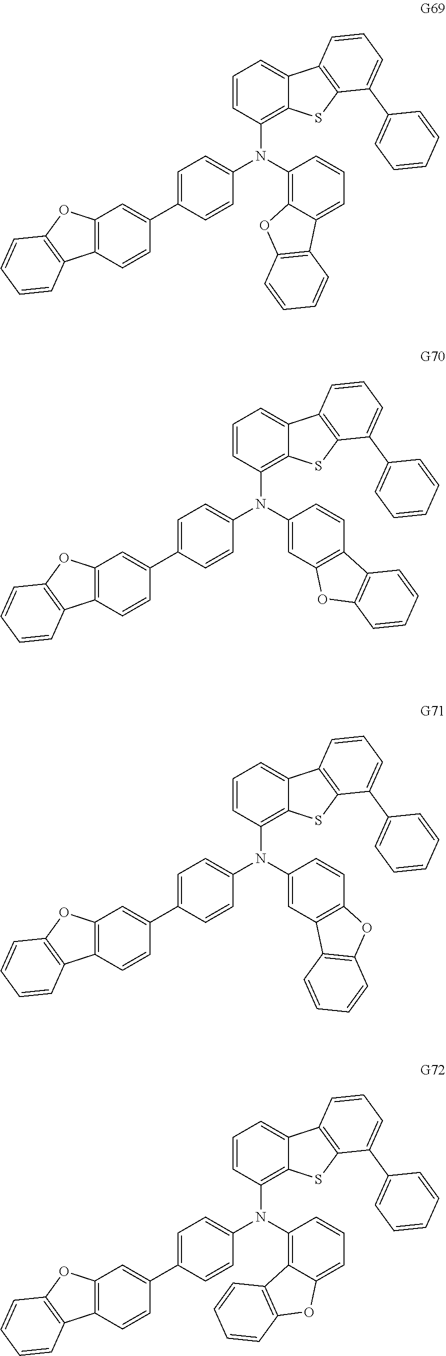 [SMILES: c1ccc(-c2cccc3c2sc2c(N(c4ccc(-c5ccc6c(c5)oc5ccccc56)cc4)c4cccc5c4oc4ccccc45)cccc23)cc1 ; c1ccc(-c2cccc3c2sc2c(N(c4ccc(-c5ccc6c(c5)oc5ccccc56)cc4)c4ccc5c(c4)oc4ccccc45)cccc23)cc1 ; c1ccc(-c2cccc3c2sc2c(N(c4ccc(-c5ccc6c(c5)oc5ccccc56)cc4)c4ccc5oc6ccccc6c5c4)cccc23)cc1 ; c1ccc(-c2cccc3c2sc2c(N(c4ccc(-c5ccc6c(c5)oc5ccccc56)cc4)c4cccc5oc6ccccc6c45)cccc23)cc1]

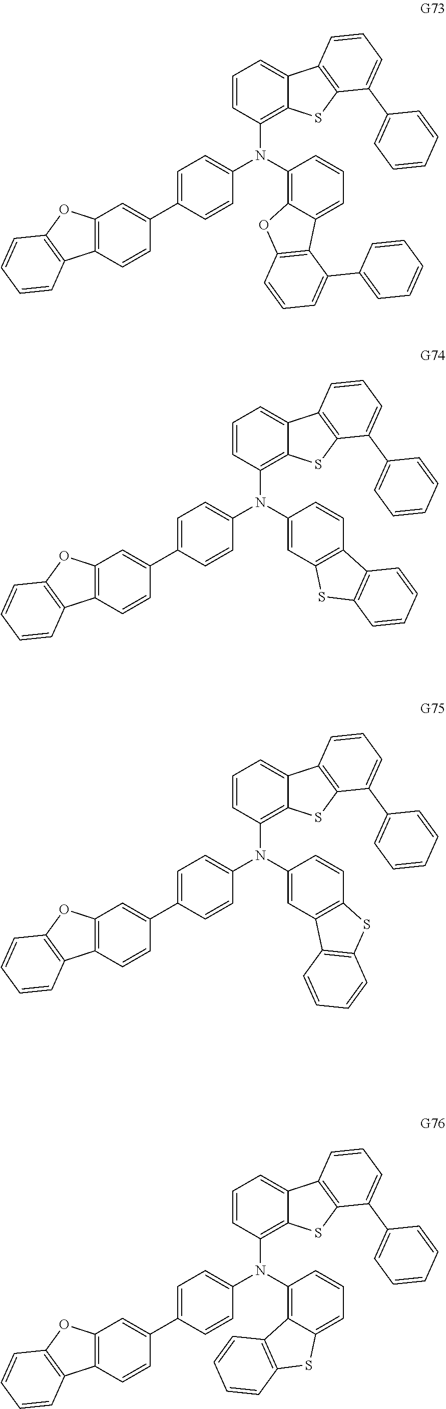 [SMILES: c1ccc(-c2cccc3c2sc2c(N(c4ccc(-c5ccc6c(c5)oc5ccccc56)cc4)c4cccc5c4oc4cccc(-c6ccccc6)c45)cccc23)cc1 ; c1ccc(-c2cccc3c2sc2c(N(c4ccc(-c5ccc6c(c5)oc5ccccc56)cc4)c4ccc5c(c4)sc4ccccc45)cccc23)cc1 ; c1ccc(-c2cccc3c2sc2c(N(c4ccc(-c5ccc6c(c5)oc5ccccc56)cc4)c4ccc5sc6ccccc6c5c4)cccc23)cc1 ; c1ccc(-c2cccc3c2sc2c(N(c4ccc(-c5ccc6c(c5)oc5ccccc56)cc4)c4cccc5sc6ccccc6c45)cccc23)cc1]

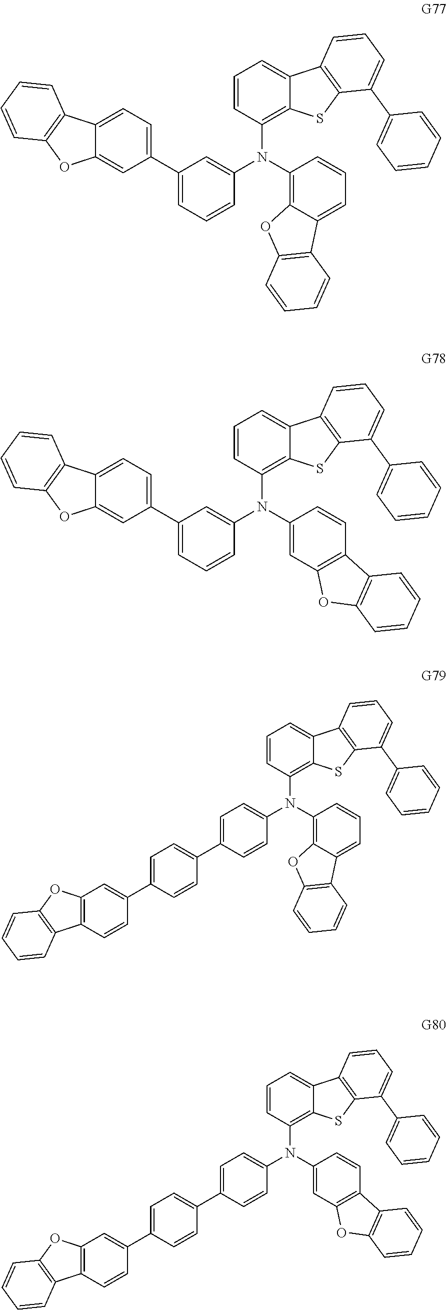 [SMILES: c1ccc(-c2cccc3c2sc2c(N(c4cccc(-c5ccc6c(c5)oc5ccccc56)c4)c4cccc5c4oc4ccccc45)cccc23)cc1 ; c1ccc(-c2cccc3c2sc2c(N(c4cccc(-c5ccc6c(c5)oc5ccccc56)c4)c4ccc5c(c4)oc4ccccc45)cccc23)cc1 ; c1ccc(-c2cccc3c2sc2c(N(c4ccc(-c5ccc(-c6ccc7c(c6)oc6ccccc67)cc5)cc4)c4cccc5c4oc4ccccc45)cccc23)cc1 ; c1ccc(-c2cccc3c2sc2c(N(c4ccc(-c5ccc(-c6ccc7c(c6)oc6ccccc67)cc5)cc4)c4ccc5c(c4)oc4ccccc45)cccc23)cc1]

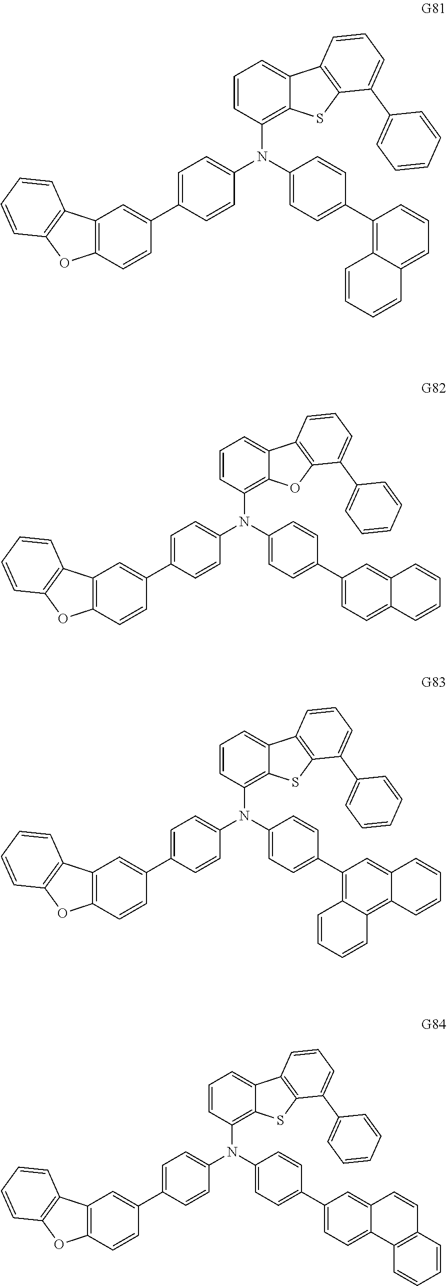 [SMILES: c1ccc(-c2cccc3c2sc2c(N(c4ccc(-c5ccc6oc7ccccc7c6c5)cc4)c4ccc(-c5cccc6ccccc56)cc4)cccc23)cc1 ; c1ccc(-c2cccc3c2oc2c(N(c4ccc(-c5ccc6ccccc6c5)cc4)c4ccc(-c5ccc6oc7ccccc7c6c5)cc4)cccc23)cc1 ; c1ccc(-c2cccc3c2sc2c(N(c4ccc(-c5ccc6oc7ccccc7c6c5)cc4)c4ccc(-c5cc6ccccc6c6ccccc56)cc4)cccc23)cc1 ; c1ccc(-c2cccc3c2sc2c(N(c4ccc(-c5ccc6c(ccc7ccccc76)c5)cc4)c4ccc(-c5ccc6oc7ccccc7c6c5)cc4)cccc23)cc1]

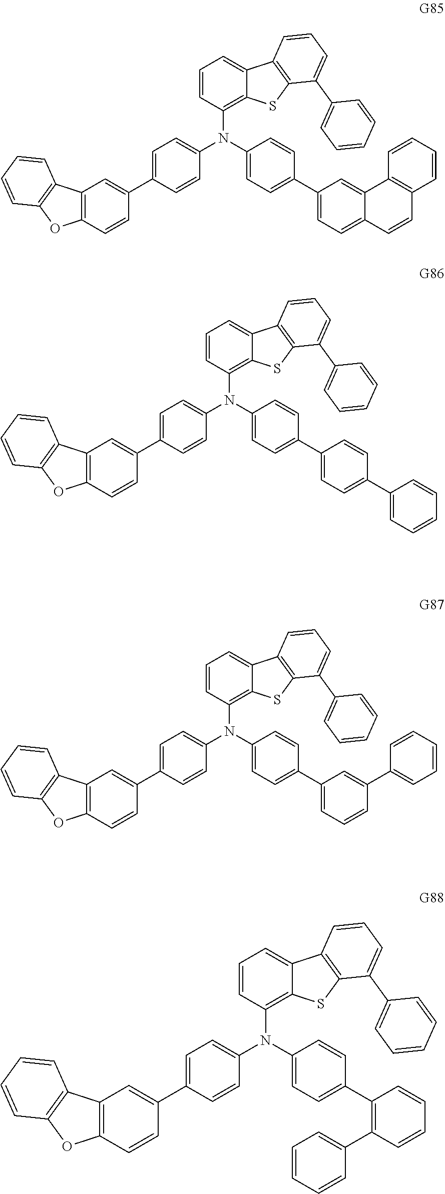 [SMILES: c1ccc(-c2cccc3c2sc2c(N(c4ccc(-c5ccc6ccc7ccccc7c6c5)cc4)c4ccc(-c5ccc6oc7ccccc7c6c5)cc4)cccc23)cc1 ; c1ccc(-c2ccc(-c3ccc(N(c4ccc(-c5ccc6oc7ccccc7c6c5)cc4)c4cccc5c4sc4c(-c6ccccc6)cccc45)cc3)cc2)cc1 ; c1ccc(-c2cccc(-c3ccc(N(c4ccc(-c5ccc6oc7ccccc7c6c5)cc4)c4cccc5c4sc4c(-c6ccccc6)cccc45)cc3)c2)cc1 ; c1ccc(-c2ccccc2-c2ccc(N(c3ccc(-c4ccc5oc6ccccc6c5c4)cc3)c3cccc4c3sc3c(-c5ccccc5)cccc34)cc2)cc1]

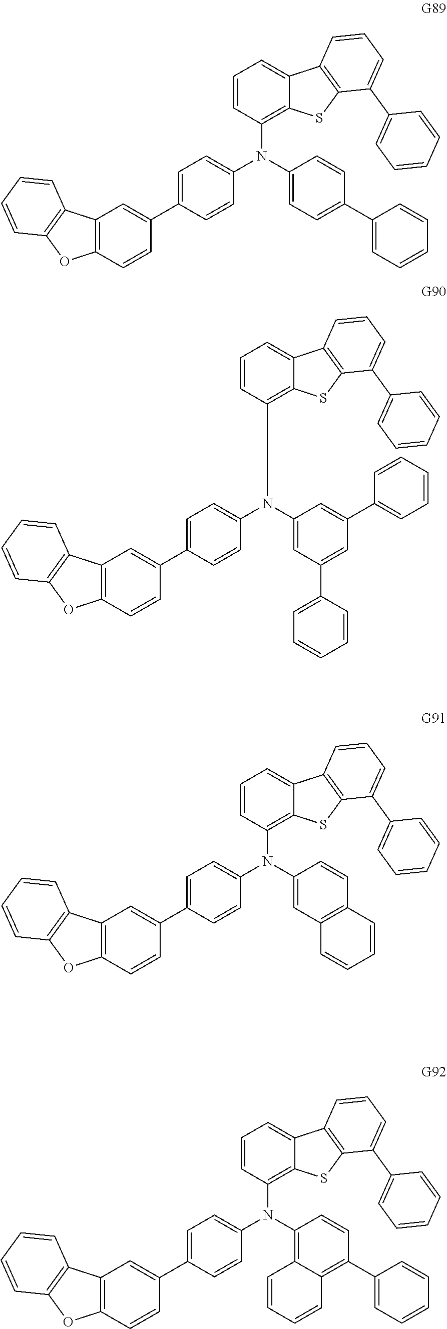 [SMILES: c1ccc(-c2ccc(N(c3ccc(-c4ccc5oc6ccccc6c5c4)cc3)c3cccc4c3sc3c(-c5ccccc5)cccc34)cc2)cc1 ; c1ccc(-c2cc(-c3ccccc3)cc(N(c3ccc(-c4ccc5oc6ccccc6c5c4)cc3)c3cccc4c3sc3c(-c5ccccc5)cccc34)c2)cc1 ; c1ccc(-c2cccc3c2sc2c(N(c4ccc(-c5ccc6oc7ccccc7c6c5)cc4)c4ccc5ccccc5c4)cccc23)cc1 ; c1ccc(-c2ccc(N(c3ccc(-c4ccc5oc6ccccc6c5c4)cc3)c3cccc4c3sc3c(-c5ccccc5)cccc34)c3ccccc23)cc1]

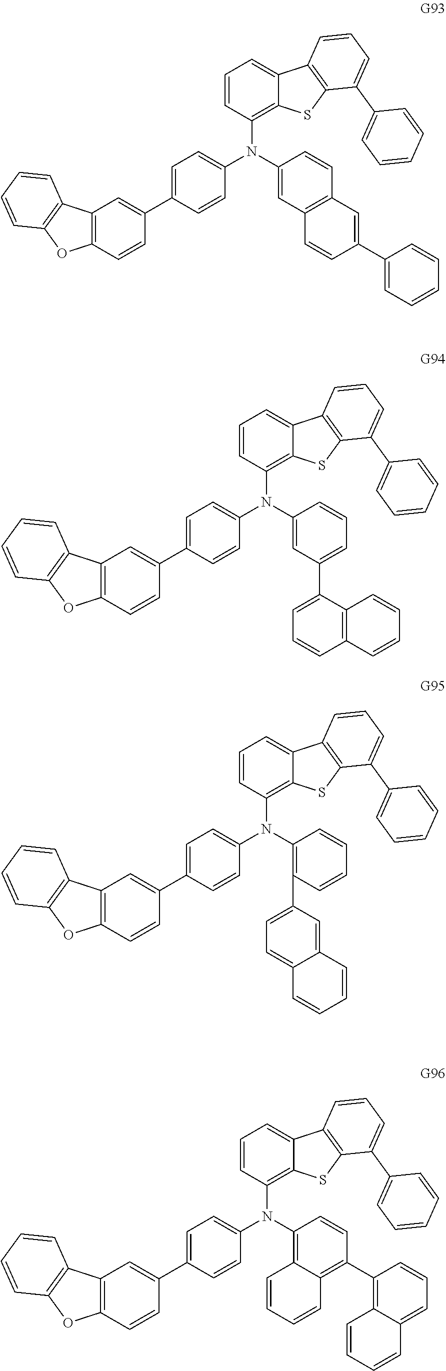 [SMILES: c1ccc(-c2ccc3cc(N(c4ccc(-c5ccc6oc7ccccc7c6c5)cc4)c4cccc5c4sc4c(-c6ccccc6)cccc45)ccc3c2)cc1 ; c1ccc(-c2cccc3c2sc2c(N(c4ccc(-c5ccc6oc7ccccc7c6c5)cc4)c4cccc(-c5cccc6ccccc56)c4)cccc23)cc1 ; c1ccc(-c2cccc3c2sc2c(N(c4ccc(-c5ccc6oc7ccccc7c6c5)cc4)c4ccccc4-c4ccc5ccccc5c4)cccc23)cc1 ; c1ccc(-c2cccc3c2sc2c(N(c4ccc(-c5ccc6oc7ccccc7c6c5)cc4)c4ccc(-c5cccc6ccccc56)c5ccccc45)cccc23)cc1]

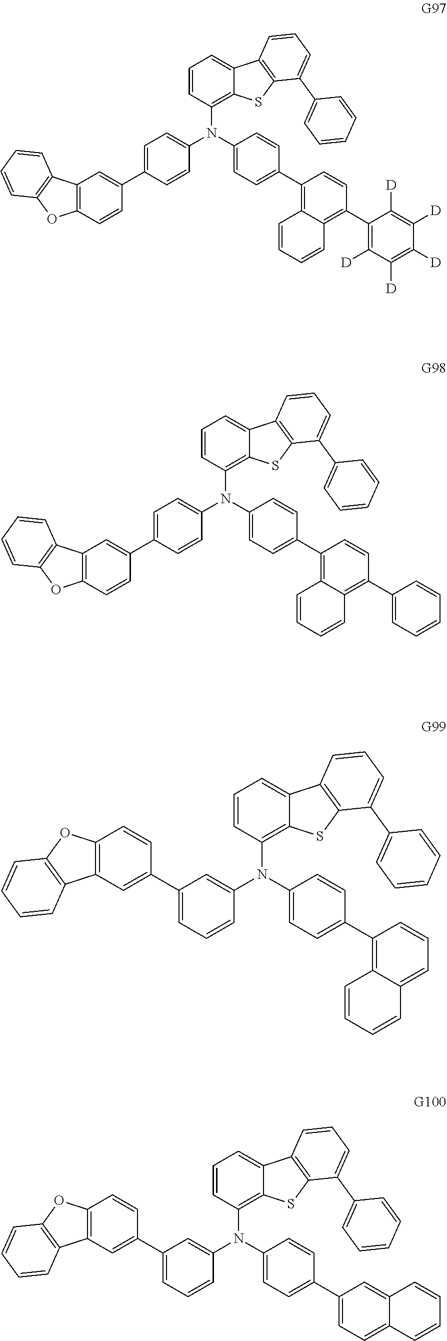 [SMILES: [2H]c1c([2H])c([2H])c(-c2ccc(-c3ccc(N(c4ccc(-c5ccc6oc7ccccc7c6c5)cc4)c4cccc5c4sc4c(-c6ccccc6)cccc45)cc3)c3ccccc23)c([2H])c1[2H] ; c1ccc(-c2ccc(-c3ccc(N(c4ccc(-c5ccc6oc7ccccc7c6c5)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4cccc(-c5ccc6oc7ccccc7c6c5)c4)cccc23)cc1 ; c1ccc(-c2cccc3c2sc2c(N(c4ccc(-c5ccc6ccccc6c5)cc4)c4cccc(-c5ccc6oc7ccccc7c6c5)c4)cccc23)cc1]

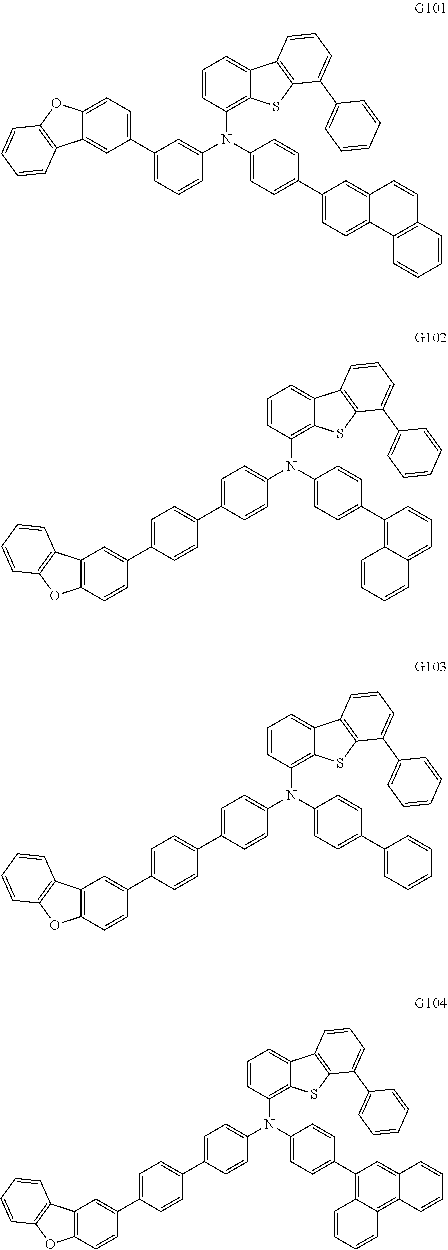 [SMILES: c1ccc(-c2cccc3c2sc2c(N(c4ccc(-c5ccc6c(ccc7ccccc76)c5)cc4)c4cccc(-c5ccc6oc7ccccc7c6c5)c4)cccc23)cc1 ; c1ccc(-c2cccc3c2sc2c(N(c4ccc(-c5ccc(-c6ccc7oc8ccccc8c7c6)cc5)cc4)c4ccc(-c5cccc6ccccc56)cc4)cccc23)cc1 ; c1ccc(-c2ccc(N(c3ccc(-c4ccc(-c5ccc6oc7ccccc7c6c5)cc4)cc3)c3cccc4c3sc3c(-c5ccccc5)cccc34)cc2)cc1 ; c1ccc(-c2cccc3c2sc2c(N(c4ccc(-c5ccc(-c6ccc7oc8ccccc8c7c6)cc5)cc4)c4ccc(-c5cc6ccccc6c6ccccc56)cc4)cccc23)cc1]

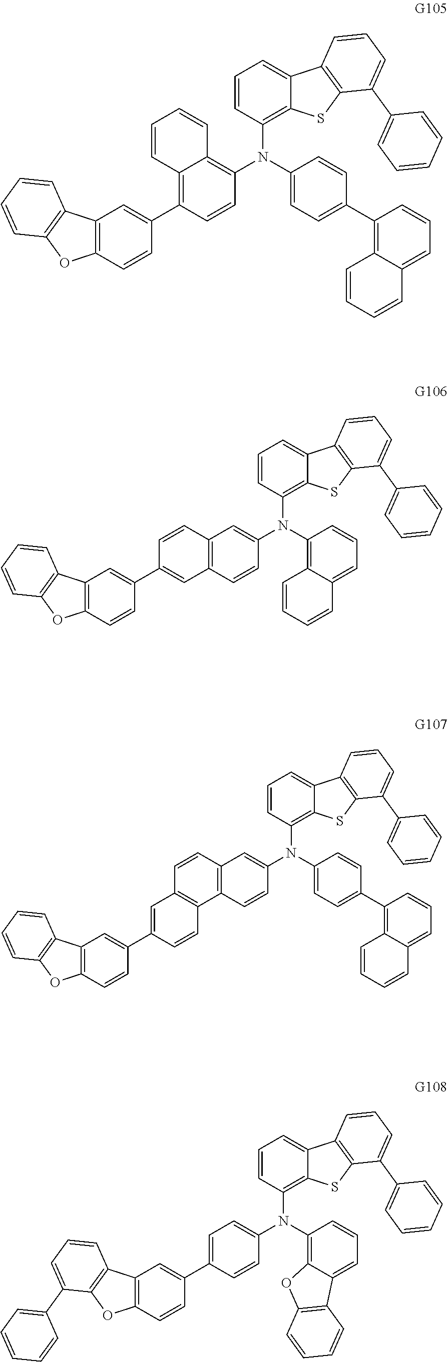 [SMILES: c1ccc(-c2cccc3c2sc2c(N(c4ccc(-c5cccc6ccccc56)cc4)c4ccc(-c5ccc6oc7ccccc7c6c5)c5ccccc45)cccc23)cc1 ; c1ccc(-c2cccc3c2sc2c(N(c4ccc5cc(-c6ccc7oc8ccccc8c7c6)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7ccc8oc9ccccc9c8c7)ccc65)c4)cccc23)cc1 ; c1ccc(-c2cccc3c2oc2ccc(-c4ccc(N(c5cccc6c5oc5ccccc56)c5cccc6c5sc5c(-c7ccccc7)cccc56)cc4)cc23)cc1]

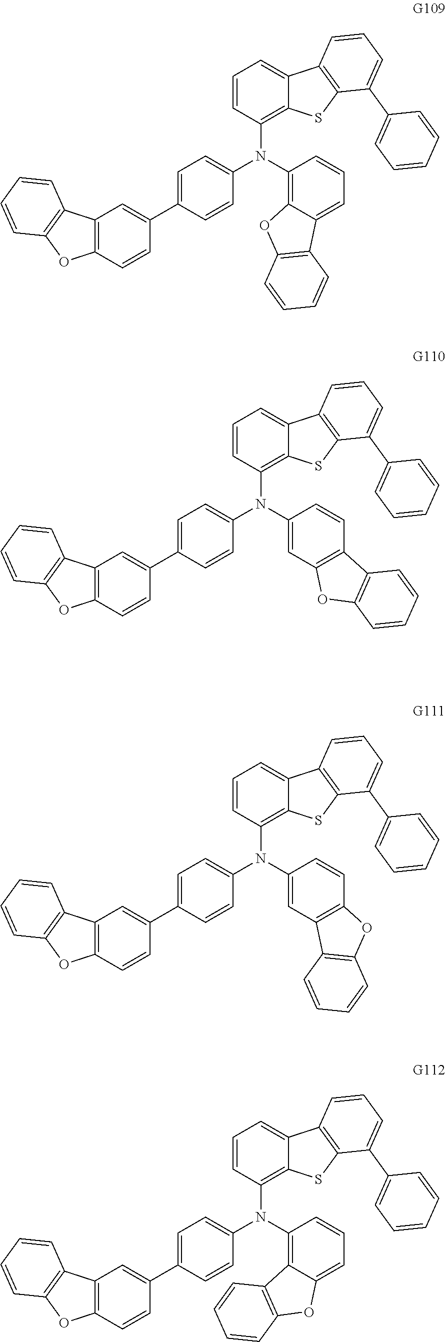 [SMILES: c1ccc(-c2cccc3c2sc2c(N(c4ccc(-c5ccc6oc7ccccc7c6c5)cc4)c4cccc5c4oc4ccccc45)cccc23)cc1 ; c1ccc(-c2cccc3c2sc2c(N(c4ccc(-c5ccc6oc7ccccc7c6c5)cc4)c4ccc5c(c4)oc4ccccc45)cccc23)cc1 ; c1ccc(-c2cccc3c2sc2c(N(c4ccc(-c5ccc6oc7ccccc7c6c5)cc4)c4ccc5oc6ccccc6c5c4)cccc23)cc1 ; c1ccc(-c2cccc3c2sc2c(N(c4ccc(-c5ccc6oc7ccccc7c6c5)cc4)c4cccc5oc6ccccc6c45)cccc23)cc1]

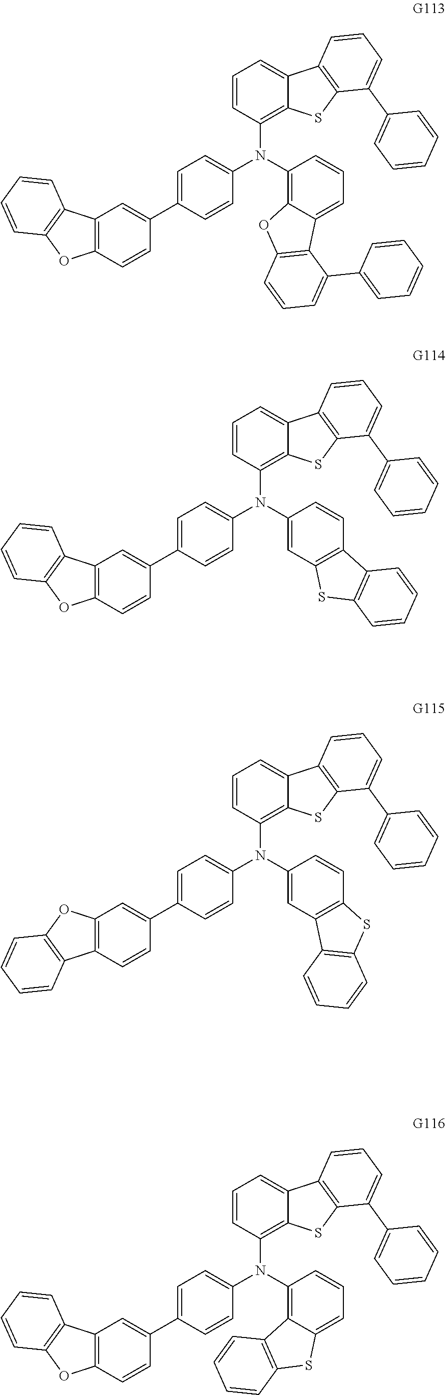 [SMILES: c1ccc(-c2cccc3c2sc2c(N(c4ccc(-c5ccc6oc7ccccc7c6c5)cc4)c4cccc5c4oc4cccc(-c6ccccc6)c45)cccc23)cc1 ; c1ccc(-c2cccc3c2sc2c(N(c4ccc(-c5ccc6oc7ccccc7c6c5)cc4)c4ccc5c(c4)sc4ccccc45)cccc23)cc1 ; c1ccc(-c2cccc3c2sc2c(N(c4ccc(-c5ccc6c(c5)oc5ccccc56)cc4)c4ccc5sc6ccccc6c5c4)cccc23)cc1 ; c1ccc(-c2cccc3c2sc2c(N(c4ccc(-c5ccc6oc7ccccc7c6c5)cc4)c4cccc5sc6ccccc6c45)cccc23)cc1]

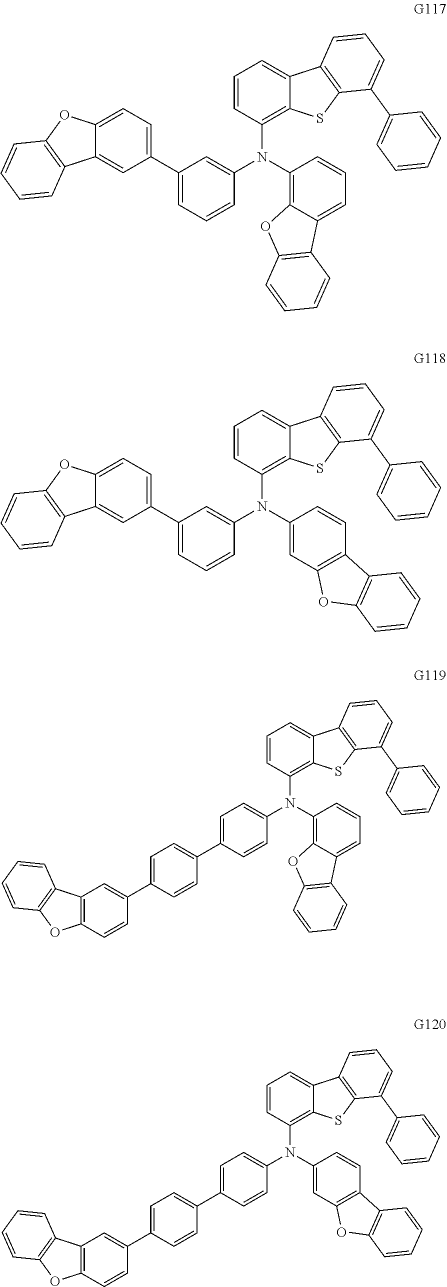 [SMILES: c1ccc(-c2cccc3c2sc2c(N(c4cccc(-c5ccc6oc7ccccc7c6c5)c4)c4cccc5c4oc4ccccc45)cccc23)cc1 ; c1ccc(-c2cccc3c2sc2c(N(c4cccc(-c5ccc6oc7ccccc7c6c5)c4)c4ccc5c(c4)oc4ccccc45)cccc23)cc1 ; c1ccc(-c2cccc3c2sc2c(N(c4ccc(-c5ccc(-c6ccc7oc8ccccc8c7c6)cc5)cc4)c4cccc5c4oc4ccccc45)cccc23)cc1 ; c1ccc(-c2cccc3c2sc2c(N(c4ccc(-c5ccc(-c6ccc7oc8ccccc8c7c6)cc5)cc4)c4ccc5c(c4)oc4ccccc45)cccc23)cc1]

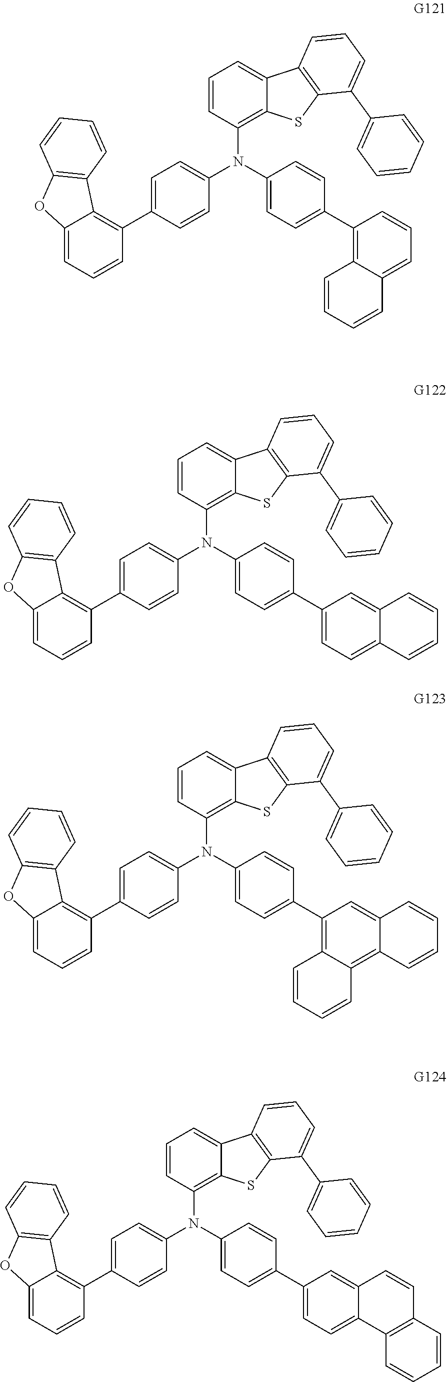 [SMILES: c1ccc(-c2cccc3c2sc2c(N(c4ccc(-c5cccc6ccccc56)cc4)c4ccc(-c5cccc6oc7ccccc7c56)cc4)cccc23)cc1 ; c1ccc(-c2cccc3c2sc2c(N(c4ccc(-c5ccc6ccccc6c5)cc4)c4ccc(-c5cccc6oc7ccccc7c56)cc4)cccc23)cc1 ; c1ccc(-c2cccc3c2sc2c(N(c4ccc(-c5cc6ccccc6c6ccccc56)cc4)c4ccc(-c5cccc6oc7ccccc7c56)cc4)cccc23)cc1 ; c1ccc(-c2cccc3c2sc2c(N(c4ccc(-c5ccc6c(ccc7ccccc76)c5)cc4)c4ccc(-c5cccc6oc7ccccc7c56)cc4)cccc23)cc1]

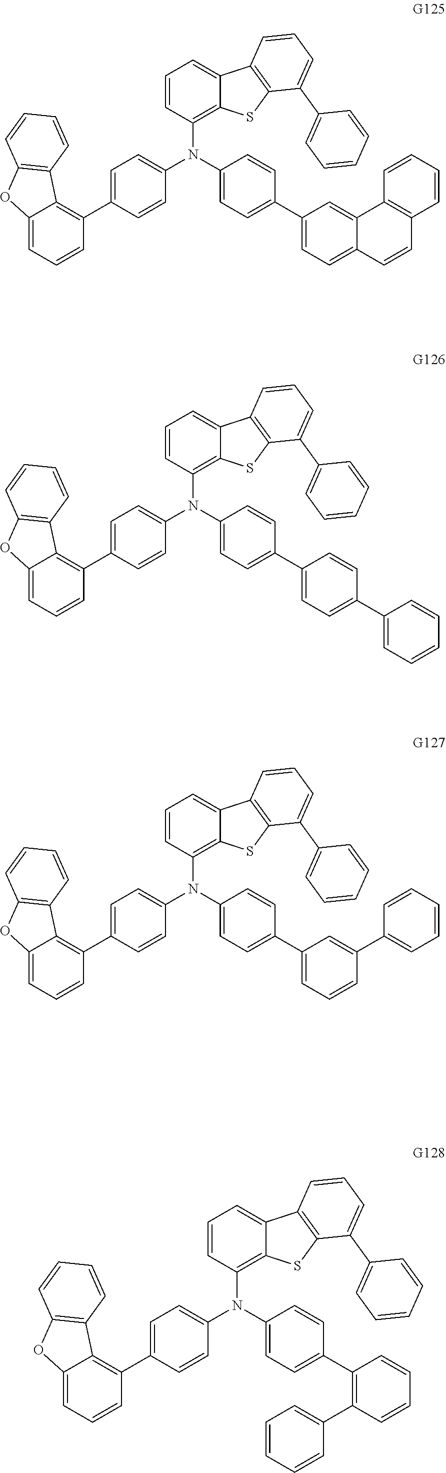 [SMILES: c1ccc(-c2cccc3c2sc2c(N(c4ccc(-c5ccc6ccc7ccccc7c6c5)cc4)c4ccc(-c5cccc6oc7ccccc7c56)cc4)cccc23)cc1 ; c1ccc(-c2ccc(-c3ccc(N(c4ccc(-c5cccc6oc7ccccc7c56)cc4)c4cccc5c4sc4c(-c6ccccc6)cccc45)cc3)cc2)cc1 ; c1ccc(-c2cccc(-c3ccc(N(c4ccc(-c5cccc6oc7ccccc7c56)cc4)c4cccc5c4sc4c(-c6ccccc6)cccc45)cc3)c2)cc1 ; c1ccc(-c2ccccc2-c2ccc(N(c3ccc(-c4cccc5oc6ccccc6c45)cc3)c3cccc4c3sc3c(-c5ccccc5)cccc34)cc2)cc1]

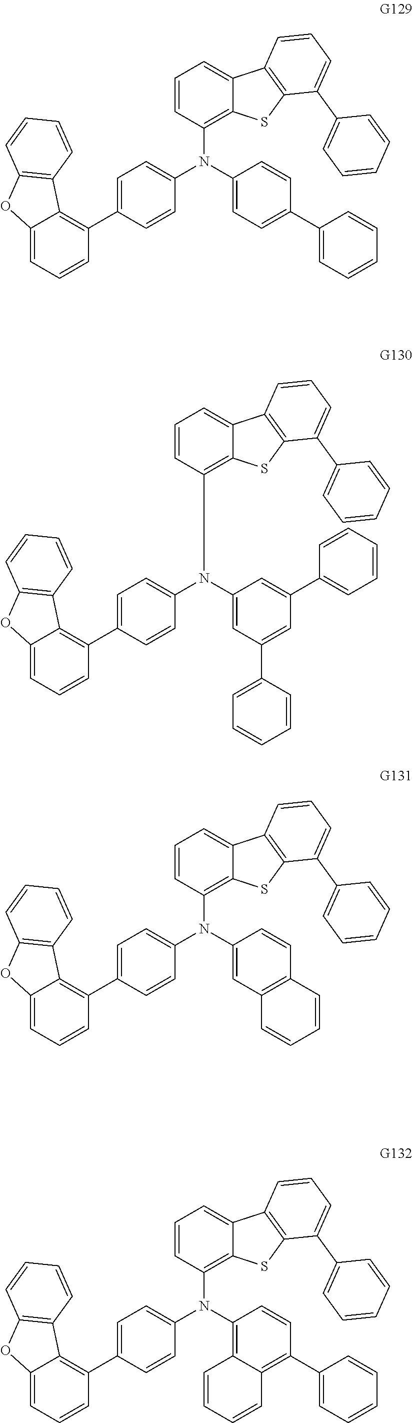 [SMILES: c1ccc(-c2ccc(N(c3ccc(-c4cccc5oc6ccccc6c45)cc3)c3cccc4c3sc3c(-c5ccccc5)cccc34)cc2)cc1 ; c1ccc(-c2cc(-c3ccccc3)cc(N(c3ccc(-c4cccc5oc6ccccc6c45)cc3)c3cccc4c3sc3c(-c5ccccc5)cccc34)c2)cc1 ; c1ccc(-c2cccc3c2sc2c(N(c4ccc(-c5cccc6oc7ccccc7c56)cc4)c4ccc5ccccc5c4)cccc23)cc1 ; c1ccc(-c2ccc(N(c3ccc(-c4cccc5oc6ccccc6c45)cc3)c3cccc4c3sc3c(-c5ccccc5)cccc34)c3ccccc23)cc1]

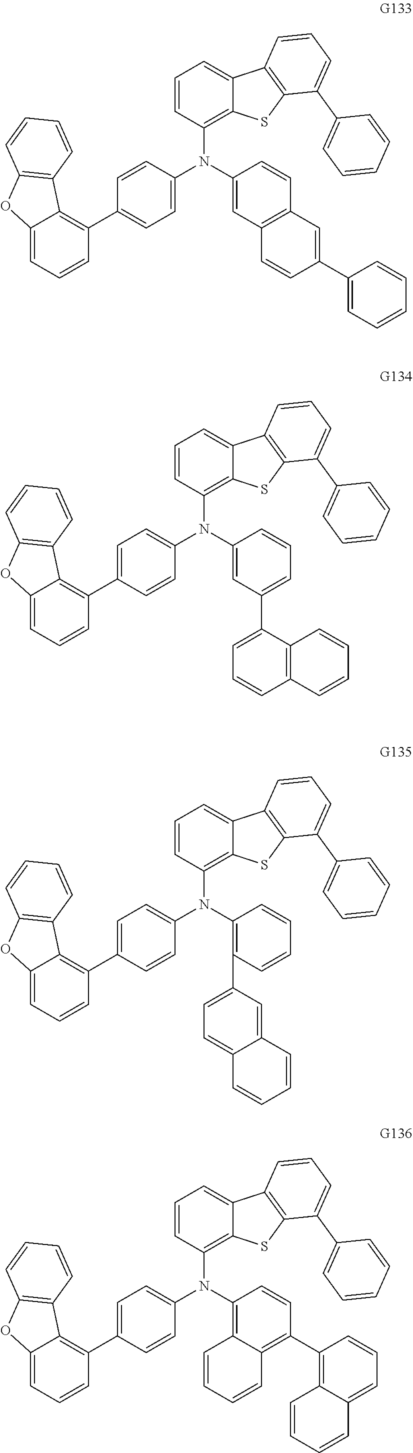 [SMILES: c1ccc(-c2ccc3cc(N(c4ccc(-c5cccc6oc7ccccc7c56)cc4)c4cccc5c4sc4c(-c6ccccc6)cccc45)ccc3c2)cc1 ; c1ccc(-c2cccc3c2sc2c(N(c4ccc(-c5cccc6oc7ccccc7c56)cc4)c4cccc(-c5cccc6ccccc56)c4)cccc23)cc1 ; c1ccc(-c2cccc3c2sc2c(N(c4ccc(-c5cccc6oc7ccccc7c56)cc4)c4ccccc4-c4ccc5ccccc5c4)cccc23)cc1 ; c1ccc(-c2cccc3c2sc2c(N(c4ccc(-c5cccc6oc7ccccc7c56)cc4)c4ccc(-c5cccc6ccccc56)c5ccccc45)cccc23)cc1]

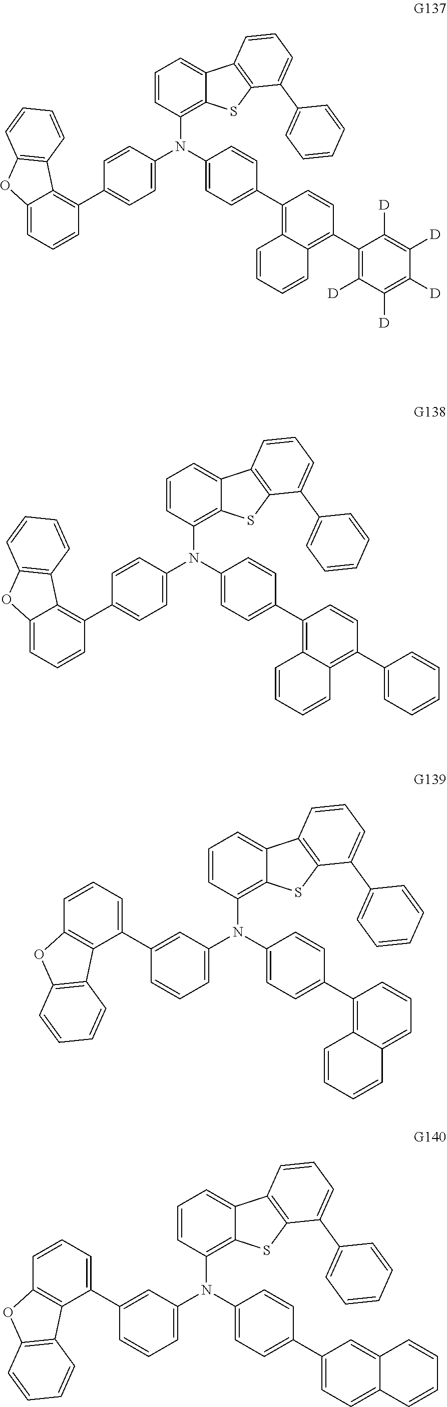 [SMILES: [2H]c1c([2H])c([2H])c(-c2ccc(-c3ccc(N(c4ccc(-c5cccc6oc7ccccc7c56)cc4)c4cccc5c4sc4c(-c6ccccc6)cccc45)cc3)c3ccccc23)c([2H])c1[2H] ; c1ccc(-c2ccc(-c3ccc(N(c4ccc(-c5cccc6oc7ccccc7c56)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4cccc(-c5cccc6oc7ccccc7c56)c4)cccc23)cc1 ; c1ccc(-c2cccc3c2sc2c(N(c4ccc(-c5ccc6ccccc6c5)cc4)c4cccc(-c5cccc6oc7ccccc7c56)c4)cccc23)cc1]

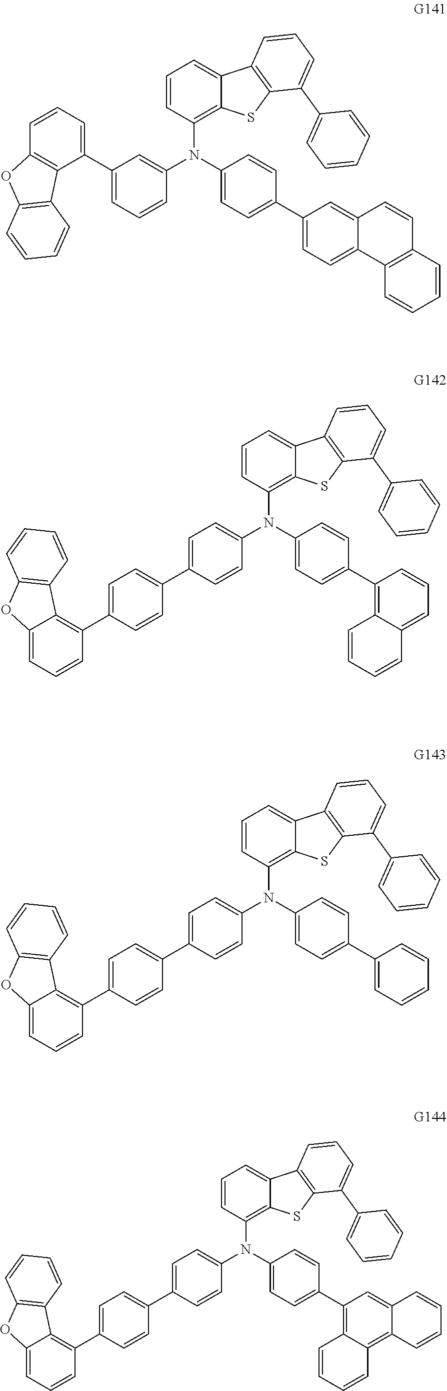 [SMILES: c1ccc(-c2cccc3c2sc2c(N(c4ccc(-c5ccc6c(ccc7ccccc76)c5)cc4)c4cccc(-c5cccc6oc7ccccc7c56)c4)cccc23)cc1 ; c1ccc(-c2cccc3c2sc2c(N(c4ccc(-c5ccc(-c6cccc7oc8ccccc8c67)cc5)cc4)c4ccc(-c5cccc6ccccc56)cc4)cccc23)cc1 ; c1ccc(-c2ccc(N(c3ccc(-c4ccc(-c5cccc6oc7ccccc7c56)cc4)cc3)c3cccc4c3sc3c(-c5ccccc5)cccc34)cc2)cc1 ; c1ccc(-c2cccc3c2sc2c(N(c4ccc(-c5ccc(-c6cccc7oc8ccccc8c67)cc5)cc4)c4ccc(-c5cc6ccccc6c6ccccc56)cc4)cccc23)cc1]

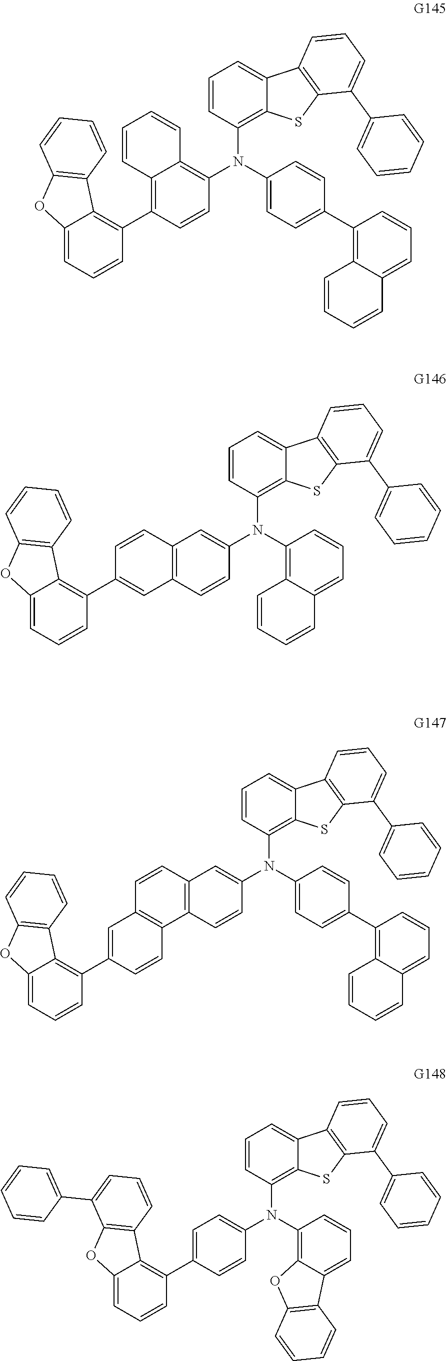 [SMILES: c1ccc(-c2cccc3c2sc2c(N(c4ccc(-c5cccc6ccccc56)cc4)c4ccc(-c5cccc6oc7ccccc7c56)c5ccccc45)cccc23)cc1 ; c1ccc(-c2cccc3c2sc2c(N(c4ccc5cc(-c6cccc7oc8ccccc8c67)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7cccc8oc9ccccc9c78)ccc65)c4)cccc23)cc1 ; c1ccc(-c2cccc3c2oc2cccc(-c4ccc(N(c5cccc6c5oc5ccccc56)c5cccc6c5sc5c(-c7ccccc7)cccc56)cc4)c23)cc1]

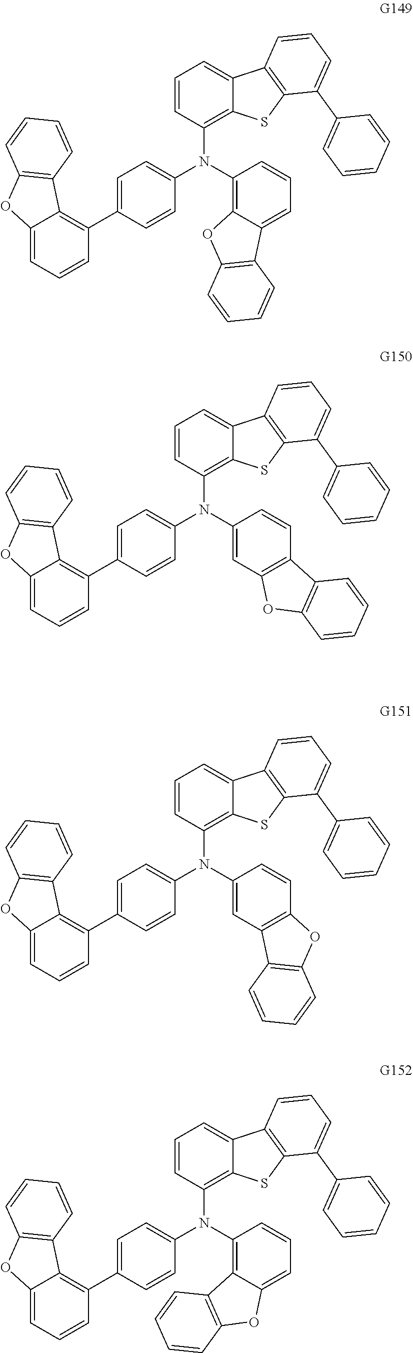 [SMILES: c1ccc(-c2cccc3c2sc2c(N(c4ccc(-c5cccc6oc7ccccc7c56)cc4)c4cccc5c4oc4ccccc45)cccc23)cc1 ; c1ccc(-c2cccc3c2sc2c(N(c4ccc(-c5cccc6oc7ccccc7c56)cc4)c4ccc5c(c4)oc4ccccc45)cccc23)cc1 ; c1ccc(-c2cccc3c2sc2c(N(c4ccc(-c5cccc6oc7ccccc7c56)cc4)c4ccc5oc6ccccc6c5c4)cccc23)cc1 ; c1ccc(-c2cccc3c2sc2c(N(c4ccc(-c5cccc6oc7ccccc7c56)cc4)c4cccc5oc6ccccc6c45)cccc23)cc1]

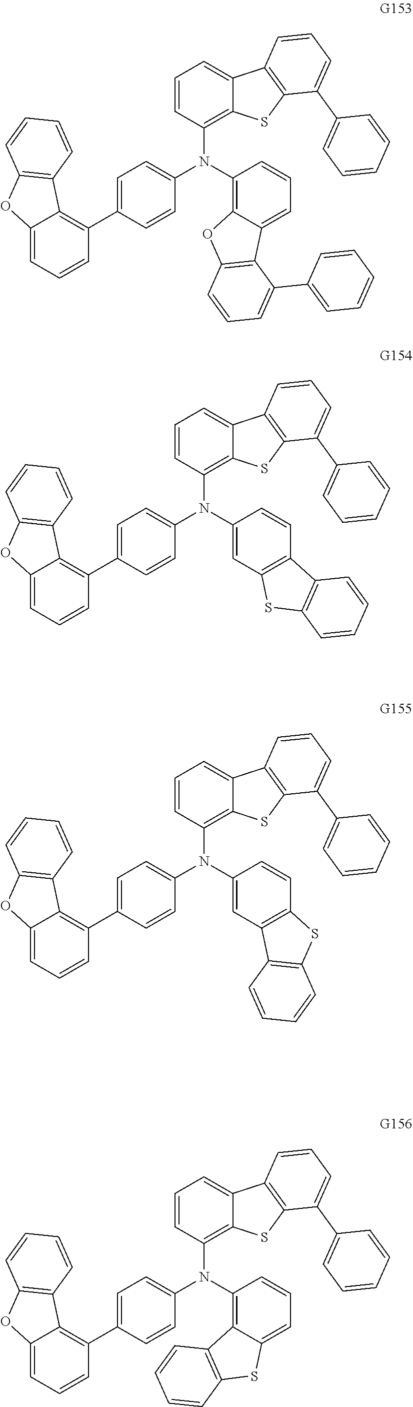 [SMILES: c1ccc(-c2cccc3c2sc2c(N(c4ccc(-c5cccc6oc7ccccc7c56)cc4)c4cccc5c4oc4cccc(-c6ccccc6)c45)cccc23)cc1 ; c1ccc(-c2cccc3c2sc2c(N(c4ccc(-c5cccc6oc7ccccc7c56)cc4)c4ccc5c(c4)sc4ccccc45)cccc23)cc1 ; c1ccc(-c2cccc3c2sc2c(N(c4ccc(-c5cccc6oc7ccccc7c56)cc4)c4ccc5sc6ccccc6c5c4)cccc23)cc1 ; c1ccc(-c2cccc3c2sc2c(N(c4ccc(-c5cccc6oc7ccccc7c56)cc4)c4cccc5sc6ccccc6c45)cccc23)cc1]

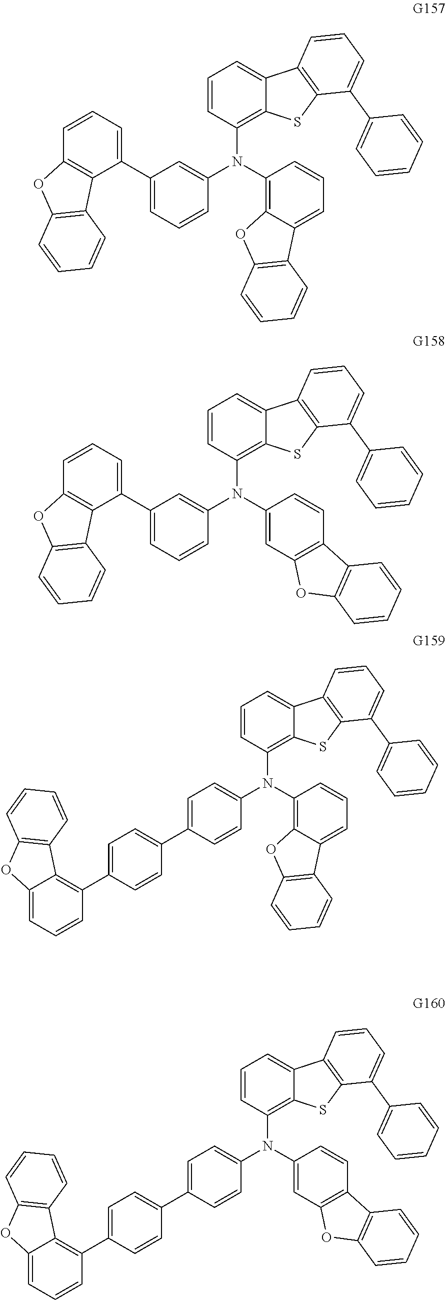 [SMILES: c1ccc(-c2cccc3c2sc2c(N(c4cccc(-c5cccc6oc7ccccc7c56)c4)c4cccc5c4oc4ccccc45)cccc23)cc1 ; c1ccc(-c2cccc3c2sc2c(N(c4cccc(-c5cccc6oc7ccccc7c56)c4)c4ccc5c(c4)oc4ccccc45)cccc23)cc1 ; c1ccc(-c2cccc3c2sc2c(N(c4ccc(-c5ccc(-c6cccc7oc8ccccc8c67)cc5)cc4)c4cccc5c4oc4ccccc45)cccc23)cc1 ; c1ccc(-c2cccc3c2sc2c(N(c4ccc(-c5ccc(-c6cccc7oc8ccccc8c67)cc5)cc4)c4ccc5c(c4)oc4ccccc45)cccc23)cc1]

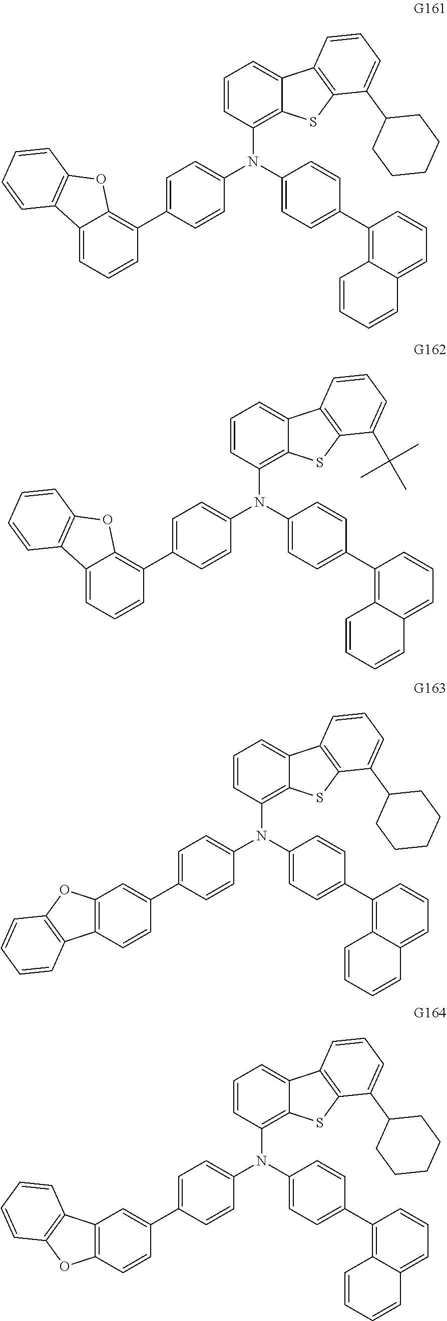

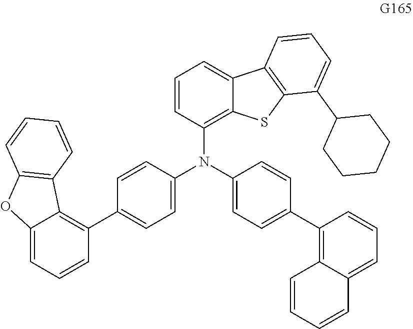 [SMILES: c1ccc2c(-c3ccc(N(c4ccc(-c5cccc6oc7ccccc7c56)cc4)c4cccc5c4sc4c(C6CCCCC6)cccc45)cc3)cccc2c1]

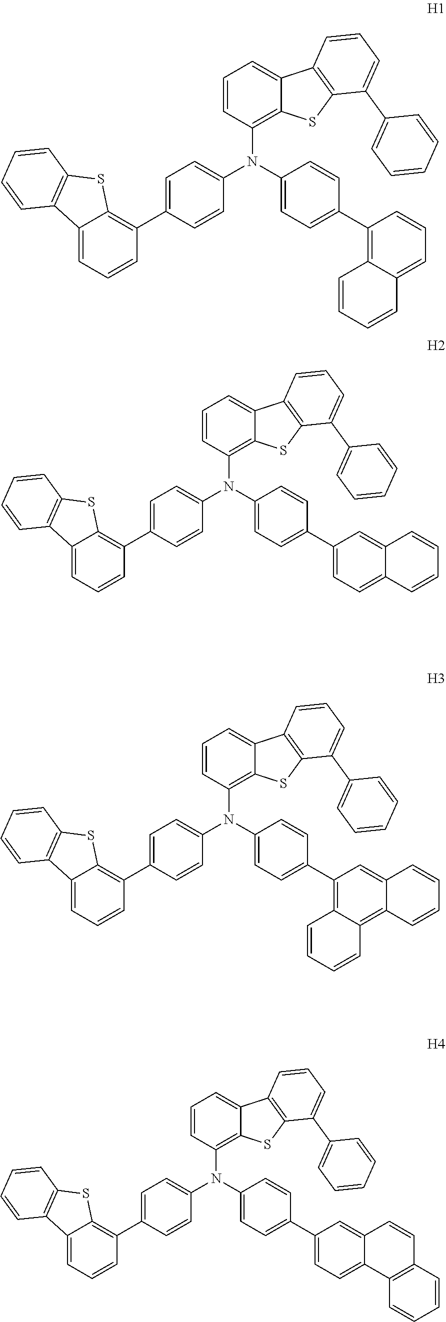 [SMILES: c1ccc(-c2cccc3c2sc2c(N(c4ccc(-c5cccc6ccccc56)cc4)c4ccc(-c5cccc6c5sc5ccccc56)cc4)cccc23)cc1 ; c1ccc(-c2cccc3c2sc2c(N(c4ccc(-c5ccc6ccccc6c5)cc4)c4ccc(-c5cccc6c5sc5ccccc56)cc4)cccc23)cc1 ; c1ccc(-c2cccc3c2sc2c(N(c4ccc(-c5cc6ccccc6c6ccccc56)cc4)c4ccc(-c5cccc6c5sc5ccccc56)cc4)cccc23)cc1 ; c1ccc(-c2cccc3c2sc2c(N(c4ccc(-c5ccc6c(ccc7ccccc76)c5)cc4)c4ccc(-c5cccc6c5sc5ccccc56)cc4)cccc23)cc1]

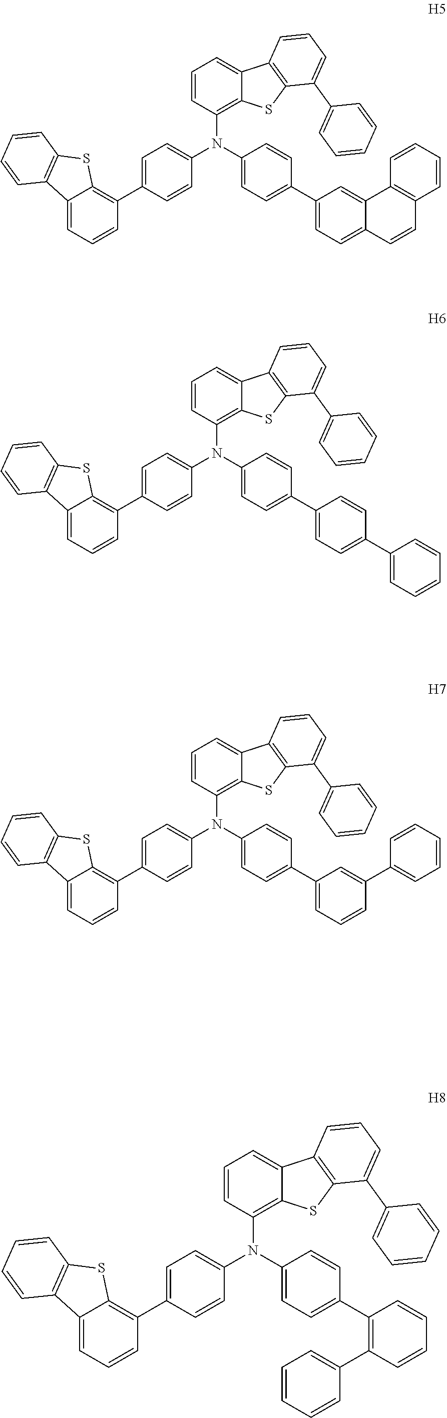 [SMILES: c1ccc(-c2cccc3c2sc2c(N(c4ccc(-c5ccc6ccc7ccccc7c6c5)cc4)c4ccc(-c5cccc6c5sc5ccccc56)cc4)cccc23)cc1 ; c1ccc(-c2ccc(-c3ccc(N(c4ccc(-c5cccc6c5sc5ccccc56)cc4)c4cccc5c4sc4c(-c6ccccc6)cccc45)cc3)cc2)cc1 ; c1ccc(-c2cccc(-c3ccc(N(c4ccc(-c5cccc6c5sc5ccccc56)cc4)c4cccc5c4sc4c(-c6ccccc6)cccc45)cc3)c2)cc1 ; c1ccc(-c2ccccc2-c2ccc(N(c3ccc(-c4cccc5c4sc4ccccc45)cc3)c3cccc4c3sc3c(-c5ccccc5)cccc34)cc2)cc1]

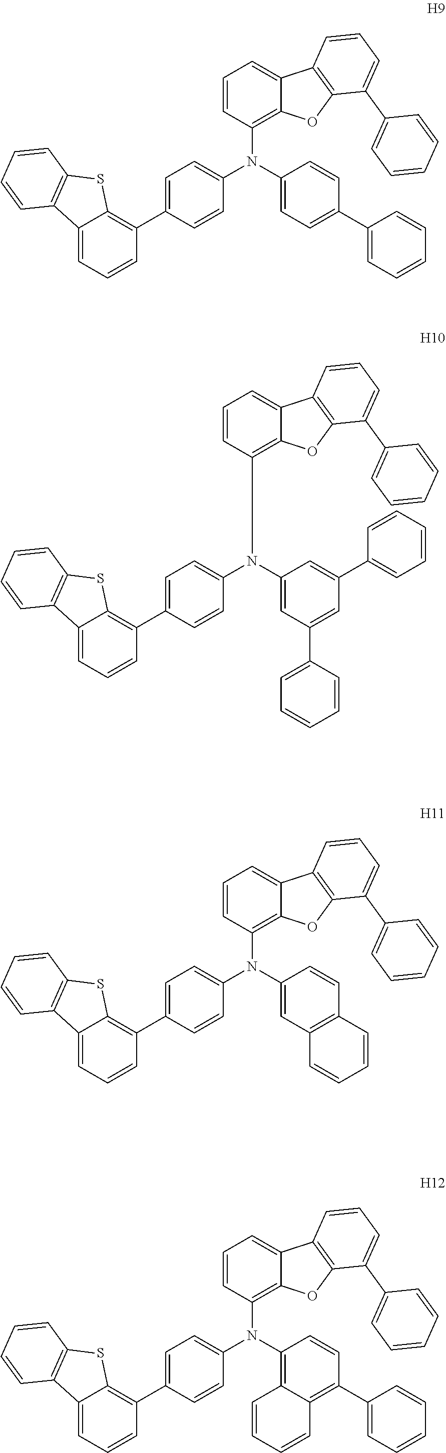 [SMILES: c1ccc(-c2ccc(N(c3ccc(-c4cccc5c4sc4ccccc45)cc3)c3cccc4c3oc3c(-c5ccccc5)cccc34)cc2)cc1 ; c1ccc(-c2cc(-c3ccccc3)cc(N(c3ccc(-c4cccc5c4sc4ccccc45)cc3)c3cccc4c3oc3c(-c5ccccc5)cccc34)c2)cc1 ; c1ccc(-c2cccc3c2oc2c(N(c4ccc(-c5cccc6c5sc5ccccc56)cc4)c4ccc5ccccc5c4)cccc23)cc1 ; c1ccc(-c2ccc(N(c3ccc(-c4cccc5c4sc4ccccc45)cc3)c3cccc4c3oc3c(-c5ccccc5)cccc34)c3ccccc23)cc1]

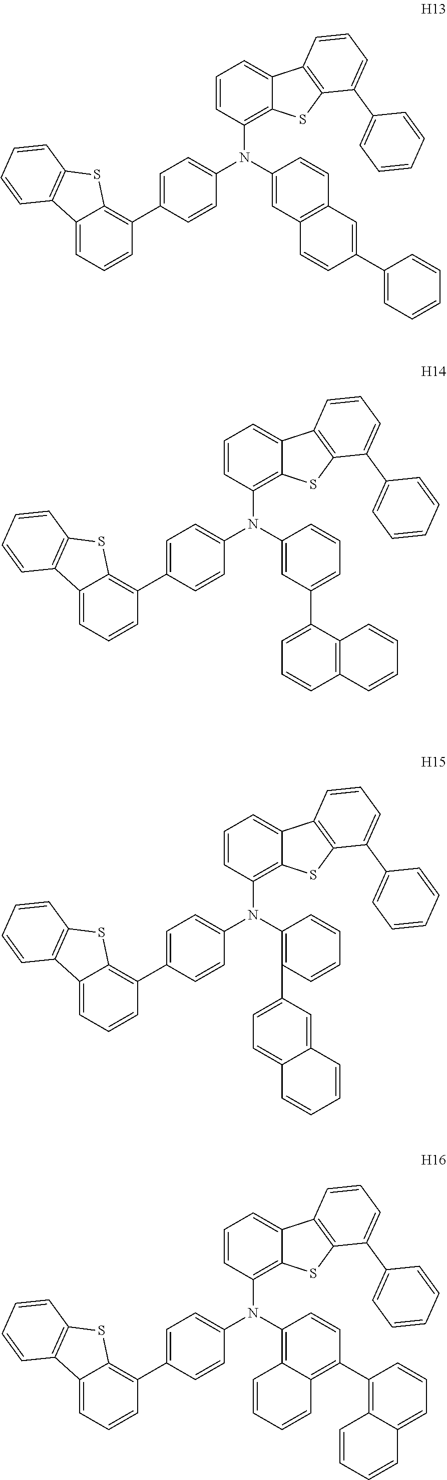 [SMILES: c1ccc(-c2ccc3cc(N(c4ccc(-c5cccc6c5sc5ccccc56)cc4)c4cccc5c4sc4c(-c6ccccc6)cccc45)ccc3c2)cc1 ; c1ccc(-c2cccc3c2sc2c(N(c4ccc(-c5cccc6c5sc5ccccc56)cc4)c4cccc(-c5cccc6ccccc56)c4)cccc23)cc1 ; c1ccc(-c2cccc3c2sc2c(N(c4ccc(-c5cccc6c5sc5ccccc56)cc4)c4ccccc4-c4ccc5ccccc5c4)cccc23)cc1 ; c1ccc(-c2cccc3c2sc2c(N(c4ccc(-c5cccc6c5sc5ccccc56)cc4)c4ccc(-c5cccc6ccccc56)c5ccccc45)cccc23)cc1]

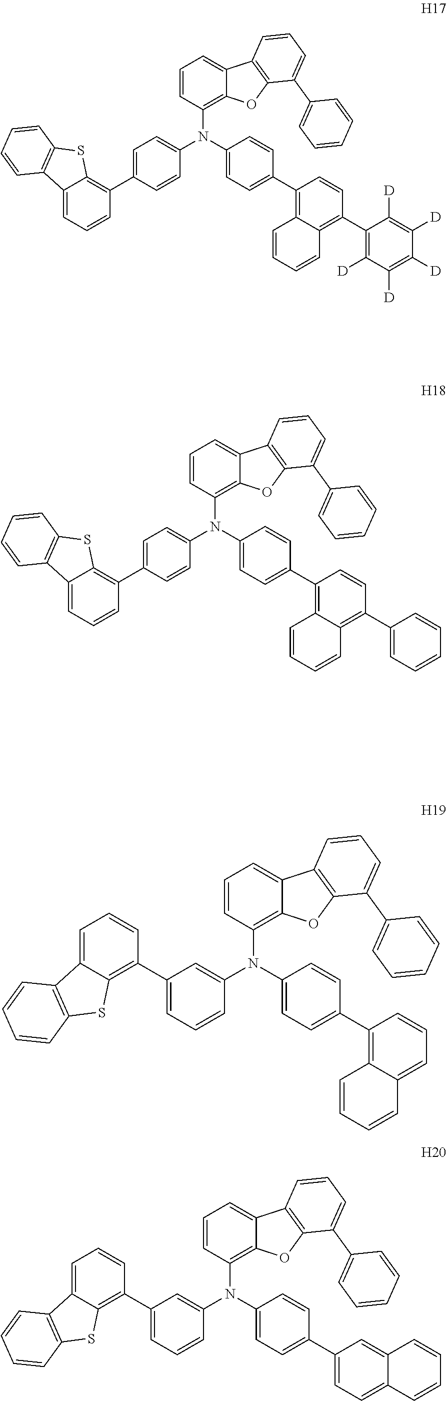 [SMILES: [2H]c1c([2H])c([2H])c(-c2ccc(-c3ccc(N(c4ccc(-c5cccc6c5sc5ccccc56)cc4)c4cccc5c4oc4c(-c6ccccc6)cccc45)cc3)c3ccccc23)c([2H])c1[2H] ; c1ccc(-c2ccc(-c3ccc(N(c4ccc(-c5cccc6c5sc5ccccc56)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5cccc6c5sc5ccccc56)c4)cccc23)cc1 ; c1ccc(-c2cccc3c2oc2c(N(c4ccc(-c5ccc6ccccc6c5)cc4)c4cccc(-c5cccc6c5sc5ccccc56)c4)cccc23)cc1]

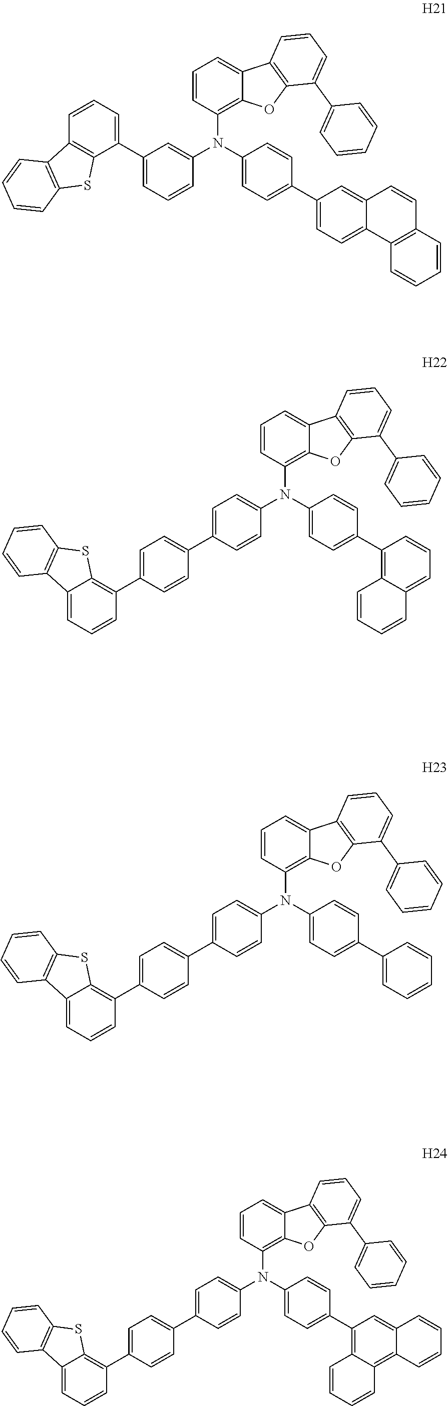 [SMILES: c1ccc(-c2cccc3c2oc2c(N(c4ccc(-c5ccc6c(ccc7ccccc76)c5)cc4)c4cccc(-c5cccc6c5sc5ccccc56)c4)cccc23)cc1 ; c1ccc(-c2cccc3c2oc2c(N(c4ccc(-c5ccc(-c6cccc7c6sc6ccccc67)cc5)cc4)c4ccc(-c5cccc6ccccc56)cc4)cccc23)cc1 ; c1ccc(-c2ccc(N(c3ccc(-c4ccc(-c5cccc6c5sc5ccccc56)cc4)cc3)c3cccc4c3oc3c(-c5ccccc5)cccc34)cc2)cc1 ; c1ccc(-c2cccc3c2oc2c(N(c4ccc(-c5ccc(-c6cccc7c6sc6ccccc67)cc5)cc4)c4ccc(-c5cc6ccccc6c6ccccc56)cc4)cccc23)cc1]

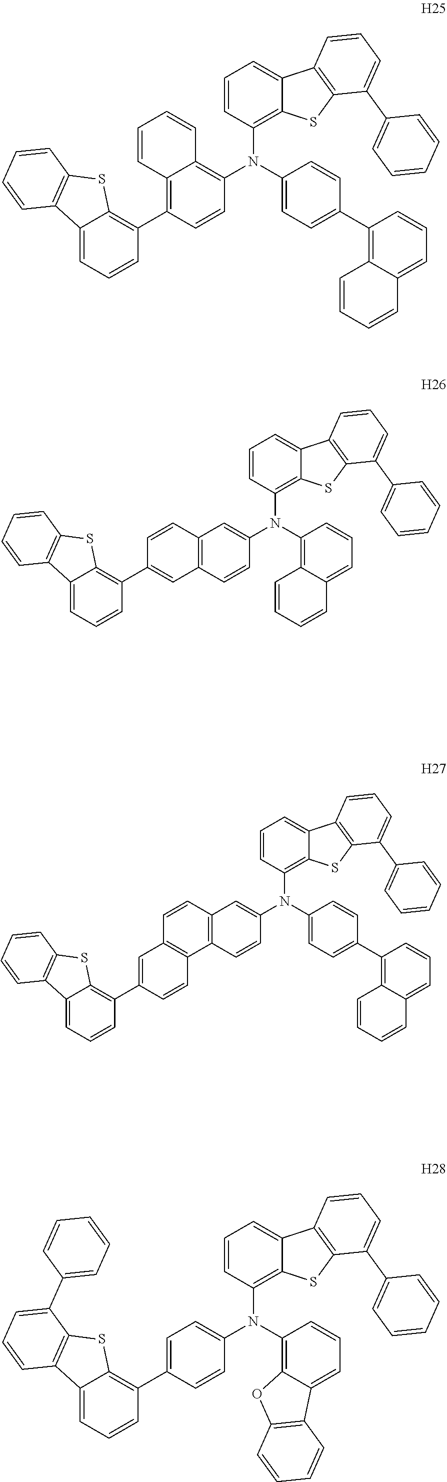

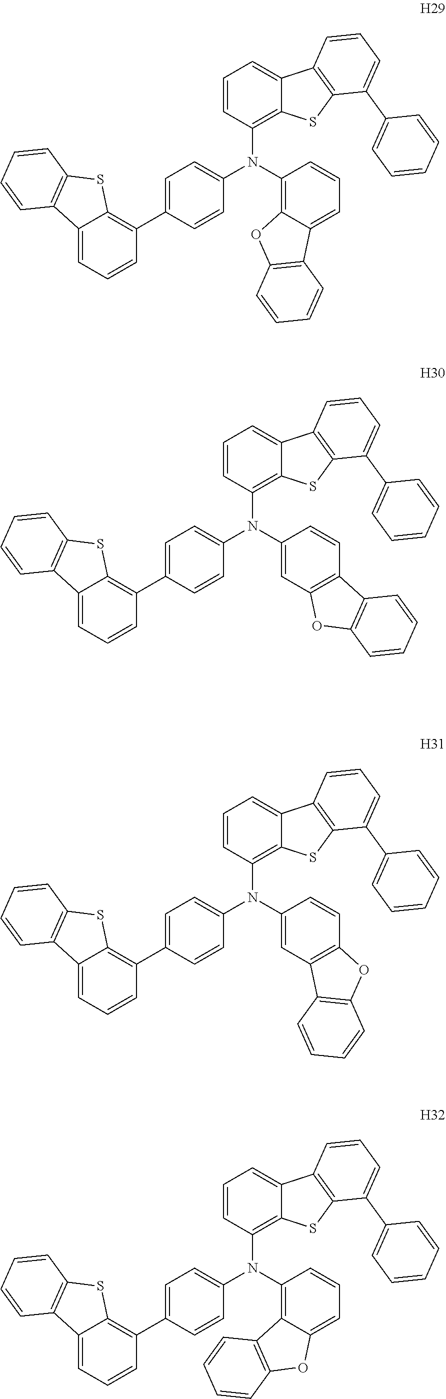 [SMILES: c1ccc(-c2cccc3c2sc2c(N(c4ccc(-c5cccc6c5sc5ccccc56)cc4)c4cccc5c4oc4ccccc45)cccc23)cc1 ; c1ccc(-c2cccc3c2sc2c(N(c4ccc(-c5cccc6c5sc5ccccc56)cc4)c4ccc5c(c4)oc4ccccc45)cccc23)cc1 ; c1ccc(-c2cccc3c2sc2c(N(c4ccc(-c5cccc6c5sc5ccccc56)cc4)c4ccc5oc6ccccc6c5c4)cccc23)cc1 ; c1ccc(-c2cccc3c2sc2c(N(c4ccc(-c5cccc6c5sc5ccccc56)cc4)c4cccc5oc6ccccc6c45)cccc23)cc1]

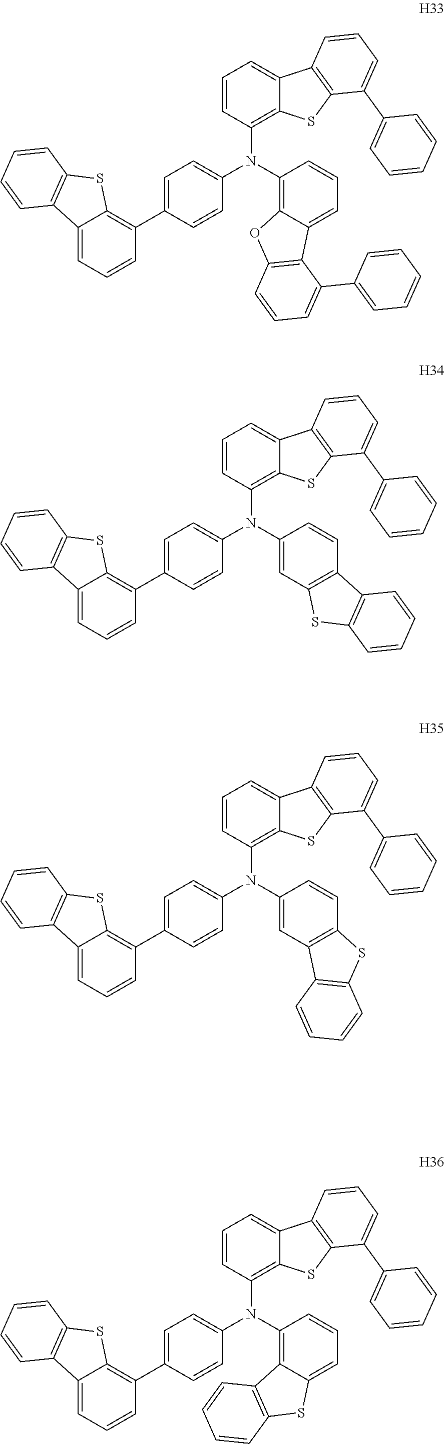 [SMILES: c1ccc(-c2cccc3c2sc2c(N(c4ccc(-c5cccc6c5sc5ccccc56)cc4)c4cccc5c4oc4cccc(-c6ccccc6)c45)cccc23)cc1 ; c1ccc(-c2cccc3c2sc2c(N(c4ccc(-c5cccc6c5sc5ccccc56)cc4)c4ccc5c(c4)sc4ccccc45)cccc23)cc1 ; c1ccc(-c2cccc3c2sc2c(N(c4ccc(-c5cccc6c5sc5ccccc56)cc4)c4ccc5sc6ccccc6c5c4)cccc23)cc1 ; c1ccc(-c2cccc3c2sc2c(N(c4ccc(-c5cccc6c5sc5ccccc56)cc4)c4cccc5sc6ccccc6c45)cccc23)cc1]

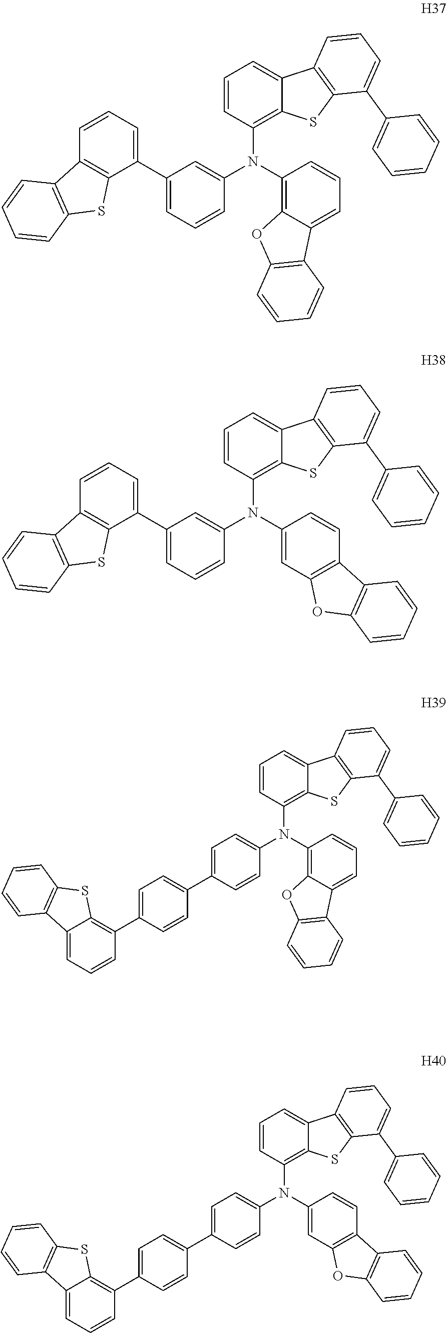 [SMILES: c1ccc(-c2cccc3c2sc2c(N(c4cccc(-c5cccc6c5sc5ccccc56)c4)c4cccc5c4oc4ccccc45)cccc23)cc1 ; c1ccc(-c2cccc3c2sc2c(N(c4cccc(-c5cccc6c5sc5ccccc56)c4)c4ccc5c(c4)oc4ccccc45)cccc23)cc1 ; c1ccc(-c2cccc3c2sc2c(N(c4ccc(-c5ccc(-c6cccc7c6sc6ccccc67)cc5)cc4)c4cccc5c4oc4ccccc45)cccc23)cc1 ; c1ccc(-c2cccc3c2sc2c(N(c4ccc(-c5ccc(-c6cccc7c6sc6ccccc67)cc5)cc4)c4ccc5c(c4)oc4ccccc45)cccc23)cc1]

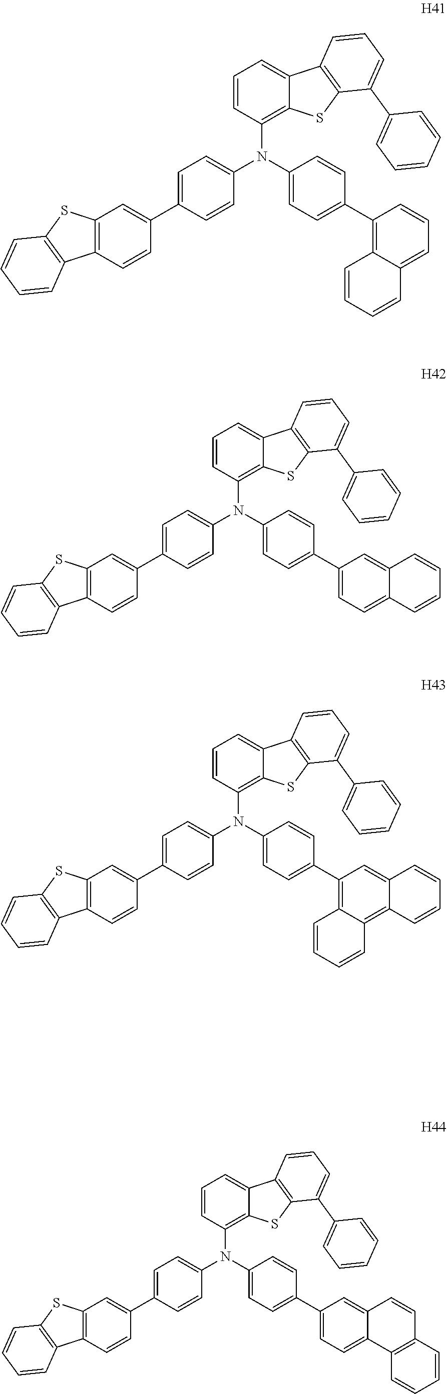 [SMILES: c1ccc(-c2cccc3c2sc2c(N(c4ccc(-c5ccc6c(c5)sc5ccccc56)cc4)c4ccc(-c5cccc6ccccc56)cc4)cccc23)cc1 ; c1ccc(-c2cccc3c2sc2c(N(c4ccc(-c5ccc6ccccc6c5)cc4)c4ccc(-c5ccc6c(c5)sc5ccccc56)cc4)cccc23)cc1 ; c1ccc(-c2cccc3c2sc2c(N(c4ccc(-c5ccc6c(c5)sc5ccccc56)cc4)c4ccc(-c5cc6ccccc6c6ccccc56)cc4)cccc23)cc1 ; c1ccc(-c2cccc3c2sc2c(N(c4ccc(-c5ccc6c(ccc7ccccc76)c5)cc4)c4ccc(-c5ccc6c(c5)sc5ccccc56)cc4)cccc23)cc1]

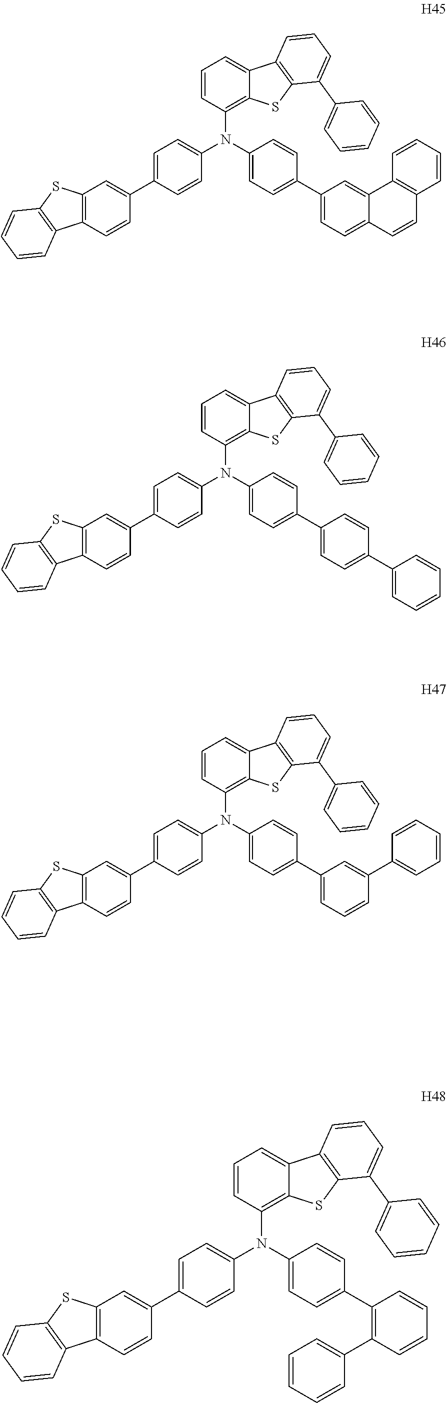 [SMILES: c1ccc(-c2cccc3c2sc2c(N(c4ccc(-c5ccc6c(c5)sc5ccccc56)cc4)c4ccc(-c5ccc6ccc7ccccc7c6c5)cc4)cccc23)cc1 ; c1ccc(-c2ccc(-c3ccc(N(c4ccc(-c5ccc6c(c5)sc5ccccc56)cc4)c4cccc5c4sc4c(-c6ccccc6)cccc45)cc3)cc2)cc1 ; c1ccc(-c2cccc(-c3ccc(N(c4ccc(-c5ccc6c(c5)sc5ccccc56)cc4)c4cccc5c4sc4c(-c6ccccc6)cccc45)cc3)c2)cc1 ; c1ccc(-c2ccccc2-c2ccc(N(c3ccc(-c4ccc5c(c4)sc4ccccc45)cc3)c3cccc4c3sc3c(-c5ccccc5)cccc34)cc2)cc1]

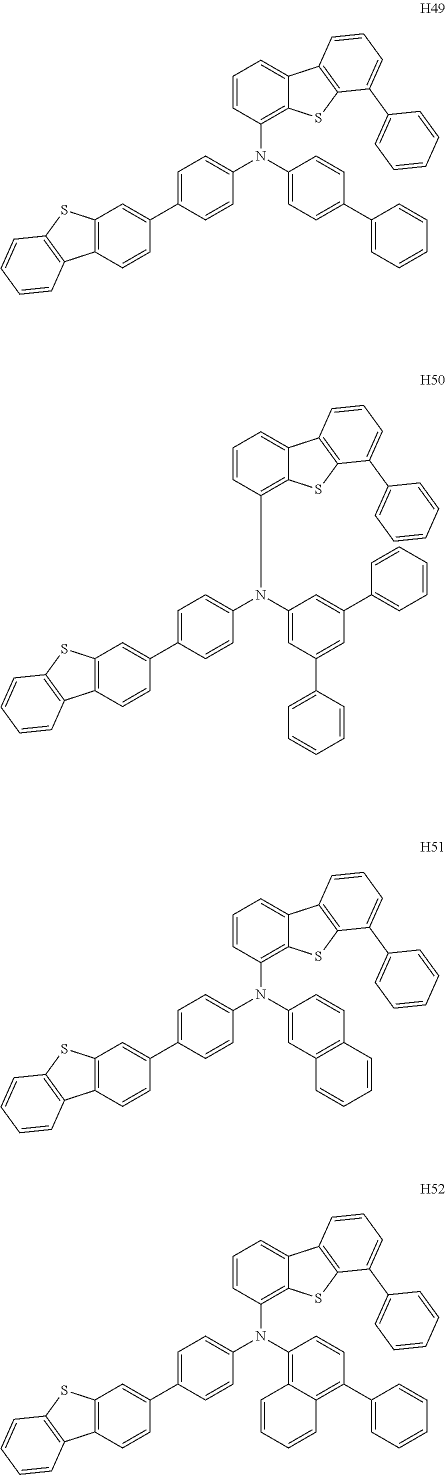 [SMILES: c1ccc(-c2ccc(N(c3ccc(-c4ccc5c(c4)sc4ccccc45)cc3)c3cccc4c3sc3c(-c5ccccc5)cccc34)cc2)cc1 ; c1ccc(-c2cc(-c3ccccc3)cc(N(c3ccc(-c4ccc5c(c4)sc4ccccc45)cc3)c3cccc4c3sc3c(-c5ccccc5)cccc34)c2)cc1 ; c1ccc(-c2cccc3c2sc2c(N(c4ccc(-c5ccc6c(c5)sc5ccccc56)cc4)c4ccc5ccccc5c4)cccc23)cc1 ; c1ccc(-c2ccc(N(c3ccc(-c4ccc5c(c4)sc4ccccc45)cc3)c3cccc4c3sc3c(-c5ccccc5)cccc34)c3ccccc23)cc1]

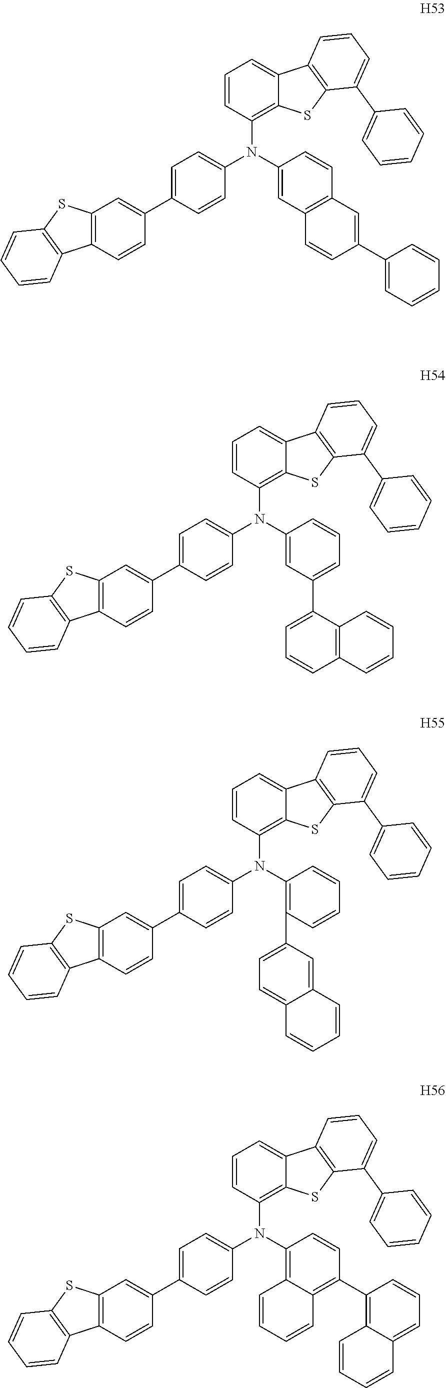 [SMILES: c1ccc(-c2ccc3cc(N(c4ccc(-c5ccc6c(c5)sc5ccccc56)cc4)c4cccc5c4sc4c(-c6ccccc6)cccc45)ccc3c2)cc1 ; c1ccc(-c2cccc3c2sc2c(N(c4ccc(-c5ccc6c(c5)sc5ccccc56)cc4)c4cccc(-c5cccc6ccccc56)c4)cccc23)cc1 ; c1ccc(-c2cccc3c2sc2c(N(c4ccc(-c5ccc6c(c5)sc5ccccc56)cc4)c4ccccc4-c4ccc5ccccc5c4)cccc23)cc1 ; c1ccc(-c2cccc3c2sc2c(N(c4ccc(-c5ccc6c(c5)sc5ccccc56)cc4)c4ccc(-c5cccc6ccccc56)c5ccccc45)cccc23)cc1]

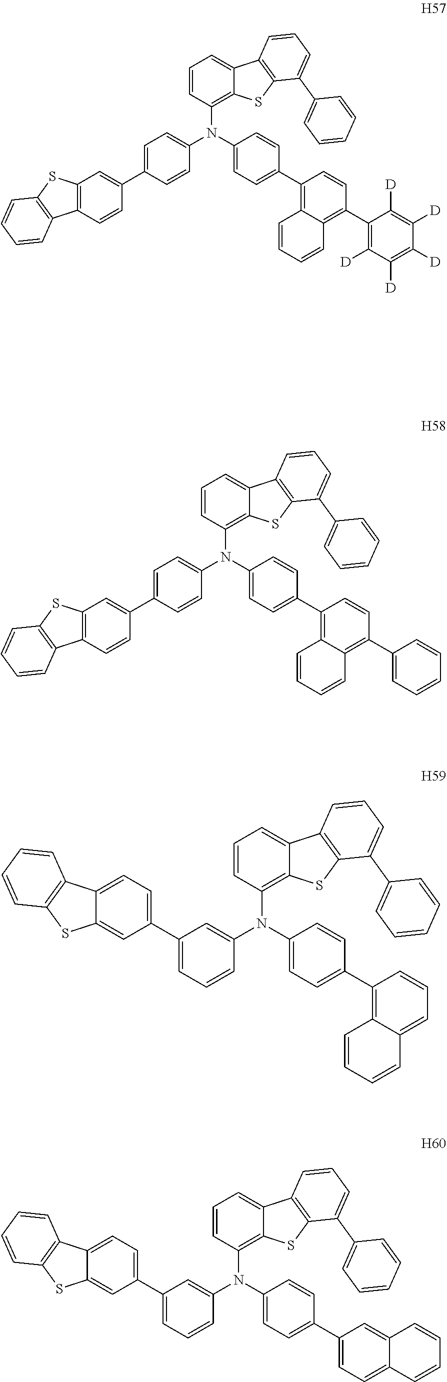 [SMILES: [2H]c1c([2H])c([2H])c(-c2ccc(-c3ccc(N(c4ccc(-c5ccc6c(c5)sc5ccccc56)cc4)c4cccc5c4sc4c(-c6ccccc6)cccc45)cc3)c3ccccc23)c([2H])c1[2H] ; c1ccc(-c2ccc(-c3ccc(N(c4ccc(-c5ccc6c(c5)sc5ccccc56)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4cccc(-c5ccc6c(c5)sc5ccccc56)c4)cccc23)cc1 ; c1ccc(-c2cccc3c2sc2c(N(c4ccc(-c5ccc6ccccc6c5)cc4)c4cccc(-c5ccc6c(c5)sc5ccccc56)c4)cccc23)cc1]

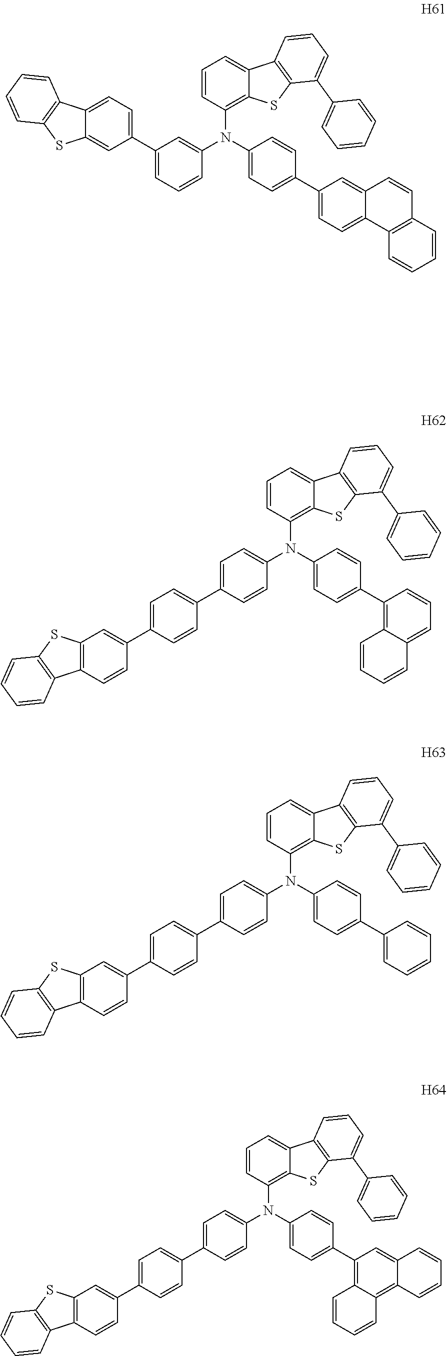 [SMILES: c1ccc(-c2cccc3c2sc2c(N(c4ccc(-c5ccc6c(ccc7ccccc76)c5)cc4)c4cccc(-c5ccc6c(c5)sc5ccccc56)c4)cccc23)cc1 ; c1ccc(-c2cccc3c2sc2c(N(c4ccc(-c5ccc(-c6ccc7c(c6)sc6ccccc67)cc5)cc4)c4ccc(-c5cccc6ccccc56)cc4)cccc23)cc1 ; c1ccc(-c2ccc(N(c3ccc(-c4ccc(-c5ccc6c(c5)sc5ccccc56)cc4)cc3)c3cccc4c3sc3c(-c5ccccc5)cccc34)cc2)cc1 ; c1ccc(-c2cccc3c2sc2c(N(c4ccc(-c5ccc(-c6ccc7c(c6)sc6ccccc67)cc5)cc4)c4ccc(-c5cc6ccccc6c6ccccc56)cc4)cccc23)cc1]

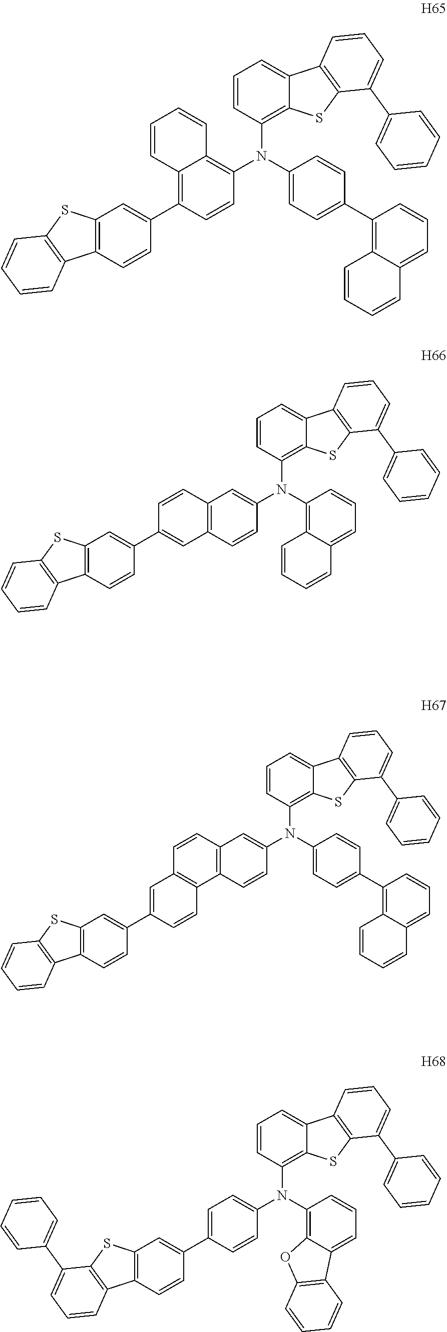 [SMILES: c1ccc(-c2cccc3c2sc2c(N(c4ccc(-c5cccc6ccccc56)cc4)c4ccc(-c5ccc6c(c5)sc5ccccc56)c5ccccc45)cccc23)cc1 ; c1ccc(-c2cccc3c2sc2c(N(c4ccc5cc(-c6ccc7c(c6)sc6ccccc67)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7ccc8c(c7)sc7ccccc78)ccc65)c4)cccc23)cc1 ; c1ccc(-c2cccc3c2sc2cc(-c4ccc(N(c5cccc6c5oc5ccccc56)c5cccc6c5sc5c(-c7ccccc7)cccc56)cc4)ccc23)cc1]

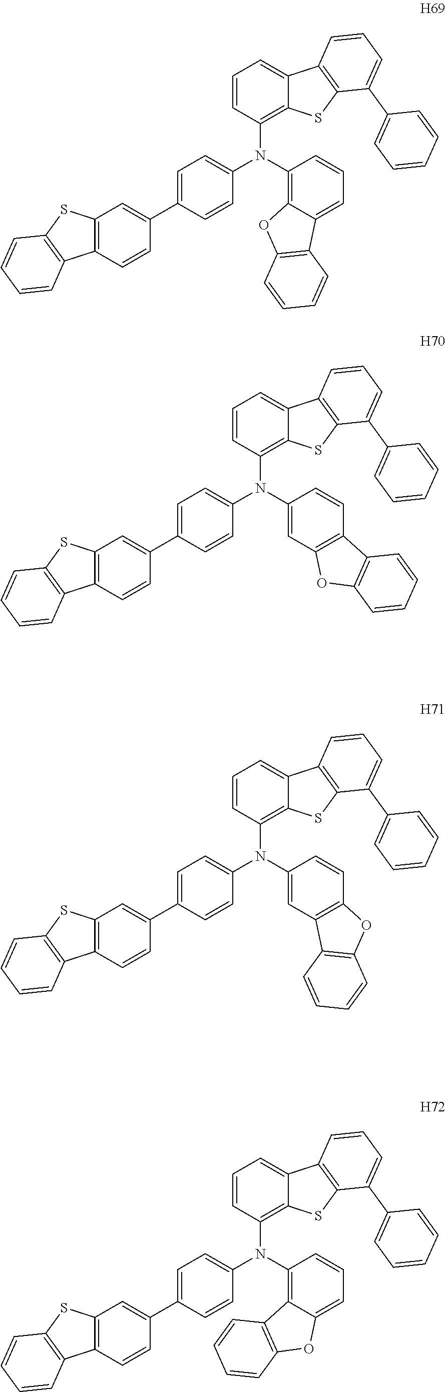 [SMILES: c1ccc(-c2cccc3c2sc2c(N(c4ccc(-c5ccc6c(c5)sc5ccccc56)cc4)c4cccc5c4oc4ccccc45)cccc23)cc1 ; c1ccc(-c2cccc3c2sc2c(N(c4ccc(-c5ccc6c(c5)sc5ccccc56)cc4)c4ccc5c(c4)oc4ccccc45)cccc23)cc1 ; c1ccc(-c2cccc3c2sc2c(N(c4ccc(-c5ccc6c(c5)sc5ccccc56)cc4)c4ccc5oc6ccccc6c5c4)cccc23)cc1 ; c1ccc(-c2cccc3c2sc2c(N(c4ccc(-c5ccc6c(c5)sc5ccccc56)cc4)c4cccc5oc6ccccc6c45)cccc23)cc1]

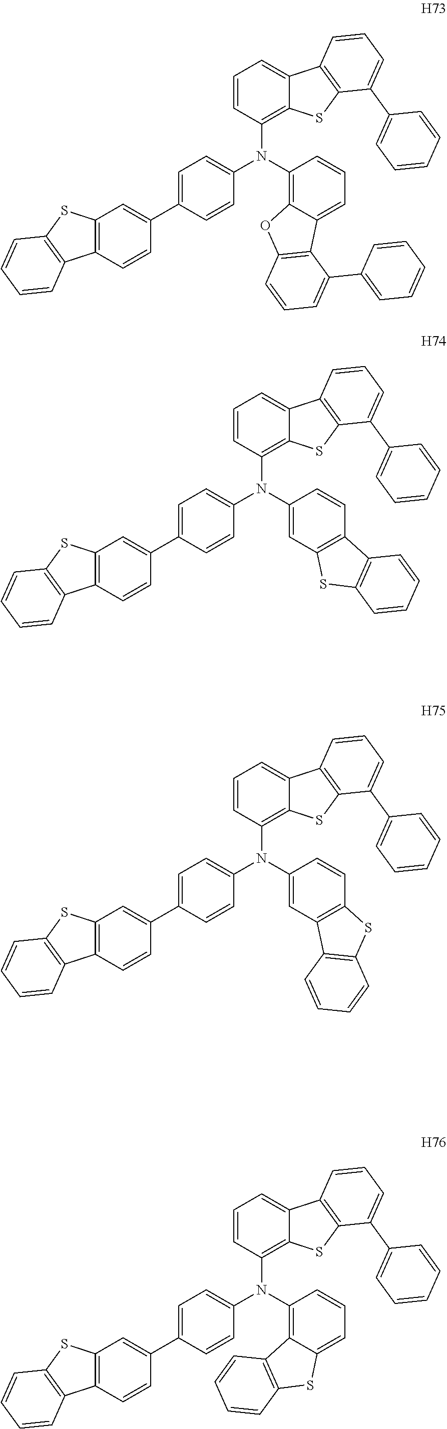 [SMILES: c1ccc(-c2cccc3c2sc2c(N(c4ccc(-c5ccc6c(c5)sc5ccccc56)cc4)c4cccc5c4oc4cccc(-c6ccccc6)c45)cccc23)cc1 ; c1ccc(-c2cccc3c2sc2c(N(c4ccc(-c5ccc6c(c5)sc5ccccc56)cc4)c4ccc5c(c4)sc4ccccc45)cccc23)cc1 ; c1ccc(-c2cccc3c2sc2c(N(c4ccc(-c5ccc6c(c5)sc5ccccc56)cc4)c4ccc5sc6ccccc6c5c4)cccc23)cc1 ; c1ccc(-c2cccc3c2sc2c(N(c4ccc(-c5ccc6c(c5)sc5ccccc56)cc4)c4cccc5sc6ccccc6c45)cccc23)cc1]

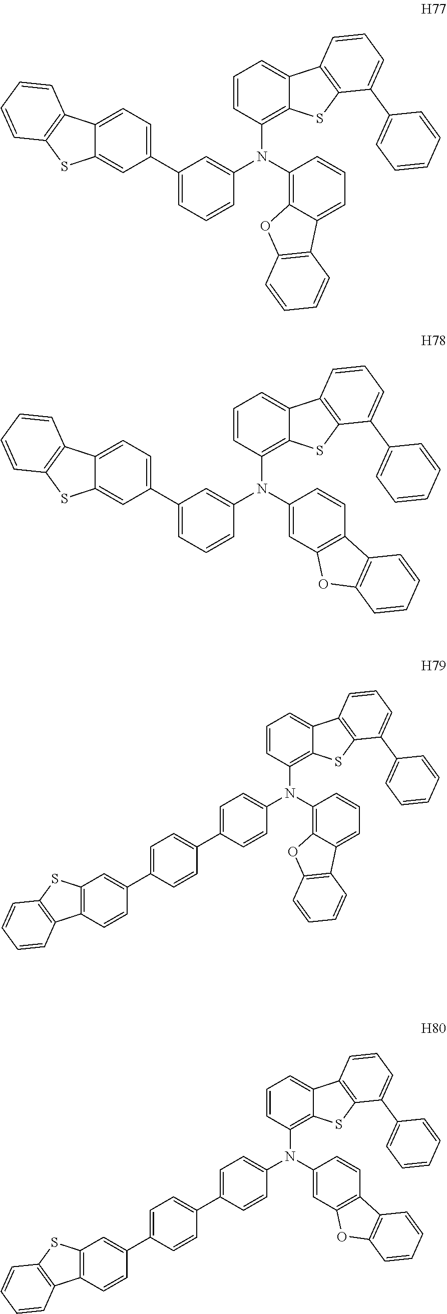 [SMILES: c1ccc(-c2cccc3c2sc2c(N(c4cccc(-c5ccc6c(c5)sc5ccccc56)c4)c4cccc5c4oc4ccccc45)cccc23)cc1 ; c1ccc(-c2cccc3c2sc2c(N(c4cccc(-c5ccc6c(c5)sc5ccccc56)c4)c4ccc5c(c4)oc4ccccc45)cccc23)cc1 ; c1ccc(-c2cccc3c2sc2c(N(c4ccc(-c5ccc(-c6ccc7c(c6)sc6ccccc67)cc5)cc4)c4cccc5c4oc4ccccc45)cccc23)cc1 ; c1ccc(-c2cccc3c2sc2c(N(c4ccc(-c5ccc(-c6ccc7c(c6)sc6ccccc67)cc5)cc4)c4ccc5c(c4)oc4ccccc45)cccc23)cc1]

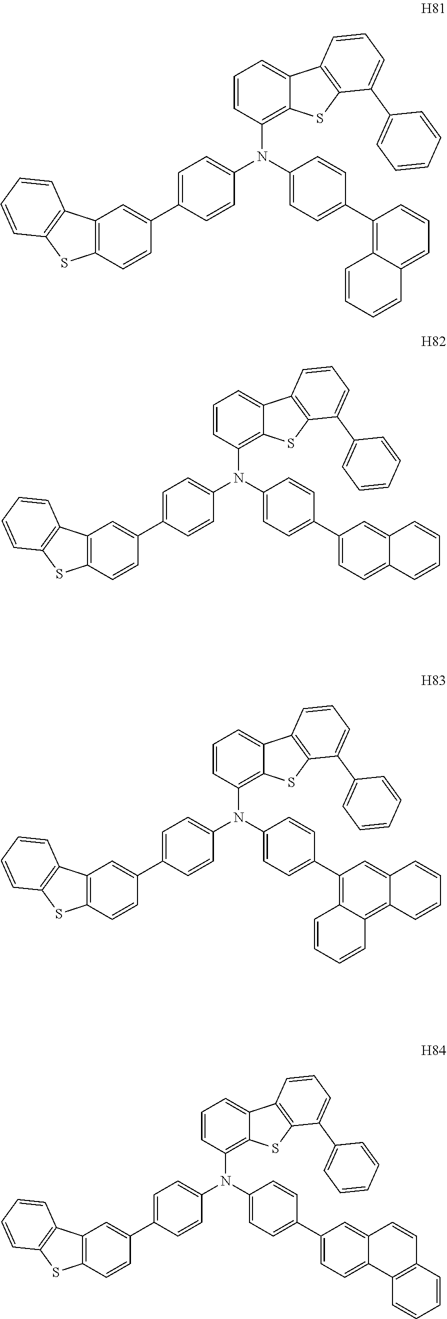 [SMILES: c1ccc(-c2cccc3c2sc2c(N(c4ccc(-c5ccc6sc7ccccc7c6c5)cc4)c4ccc(-c5cccc6ccccc56)cc4)cccc23)cc1 ; c1ccc(-c2cccc3c2sc2c(N(c4ccc(-c5ccc6ccccc6c5)cc4)c4ccc(-c5ccc6sc7ccccc7c6c5)cc4)cccc23)cc1 ; c1ccc(-c2cccc3c2sc2c(N(c4ccc(-c5ccc6sc7ccccc7c6c5)cc4)c4ccc(-c5cc6ccccc6c6ccccc56)cc4)cccc23)cc1 ; c1ccc(-c2cccc3c2sc2c(N(c4ccc(-c5ccc6c(ccc7ccccc76)c5)cc4)c4ccc(-c5ccc6sc7ccccc7c6c5)cc4)cccc23)cc1]

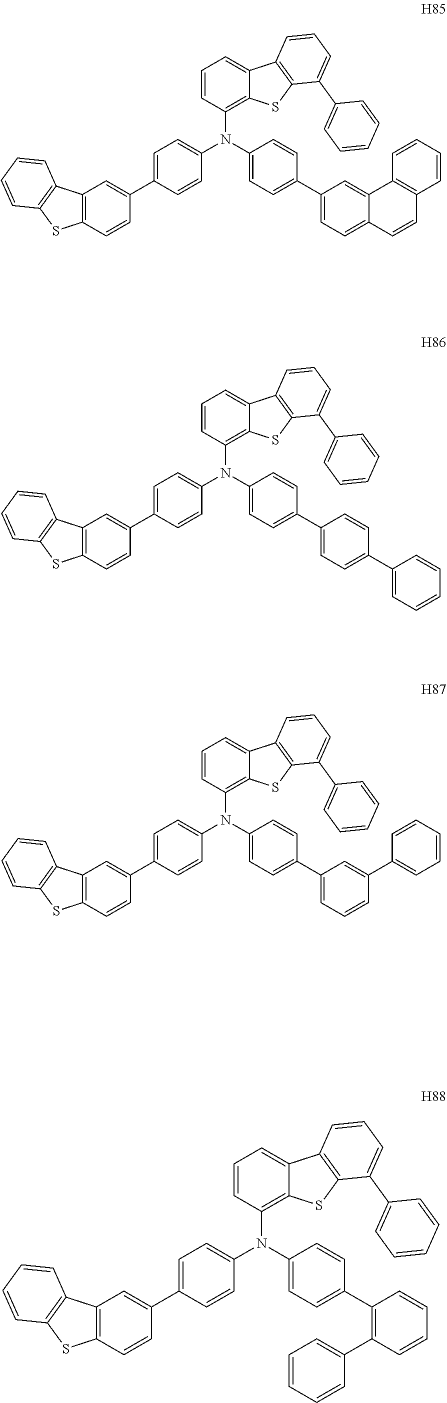 [SMILES: c1ccc(-c2cccc3c2sc2c(N(c4ccc(-c5ccc6ccc7ccccc7c6c5)cc4)c4ccc(-c5ccc6sc7ccccc7c6c5)cc4)cccc23)cc1 ; c1ccc(-c2ccc(-c3ccc(N(c4ccc(-c5ccc6sc7ccccc7c6c5)cc4)c4cccc5c4sc4c(-c6ccccc6)cccc45)cc3)cc2)cc1 ; c1ccc(-c2cccc(-c3ccc(N(c4ccc(-c5ccc6sc7ccccc7c6c5)cc4)c4cccc5c4sc4c(-c6ccccc6)cccc45)cc3)c2)cc1 ; c1ccc(-c2ccccc2-c2ccc(N(c3ccc(-c4ccc5sc6ccccc6c5c4)cc3)c3cccc4c3sc3c(-c5ccccc5)cccc34)cc2)cc1]

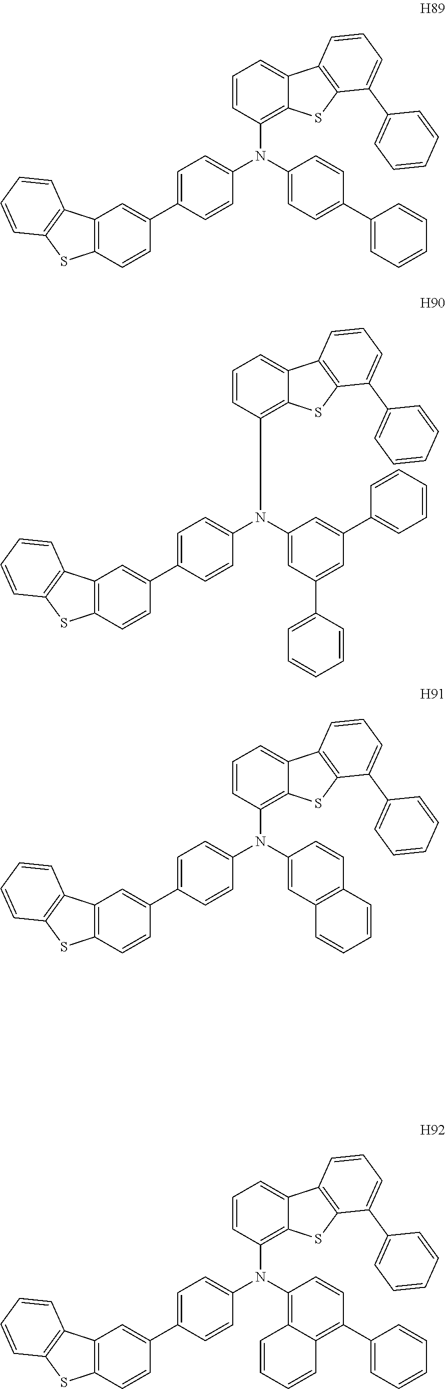 [SMILES: c1ccc(-c2ccc(N(c3ccc(-c4ccc5sc6ccccc6c5c4)cc3)c3cccc4c3sc3c(-c5ccccc5)cccc34)cc2)cc1 ; c1ccc(-c2cc(-c3ccccc3)cc(N(c3ccc(-c4ccc5sc6ccccc6c5c4)cc3)c3cccc4c3sc3c(-c5ccccc5)cccc34)c2)cc1 ; c1ccc(-c2cccc3c2sc2c(N(c4ccc(-c5ccc6sc7ccccc7c6c5)cc4)c4ccc5ccccc5c4)cccc23)cc1 ; c1ccc(-c2ccc(N(c3ccc(-c4ccc5sc6ccccc6c5c4)cc3)c3cccc4c3sc3c(-c5ccccc5)cccc34)c3ccccc23)cc1]

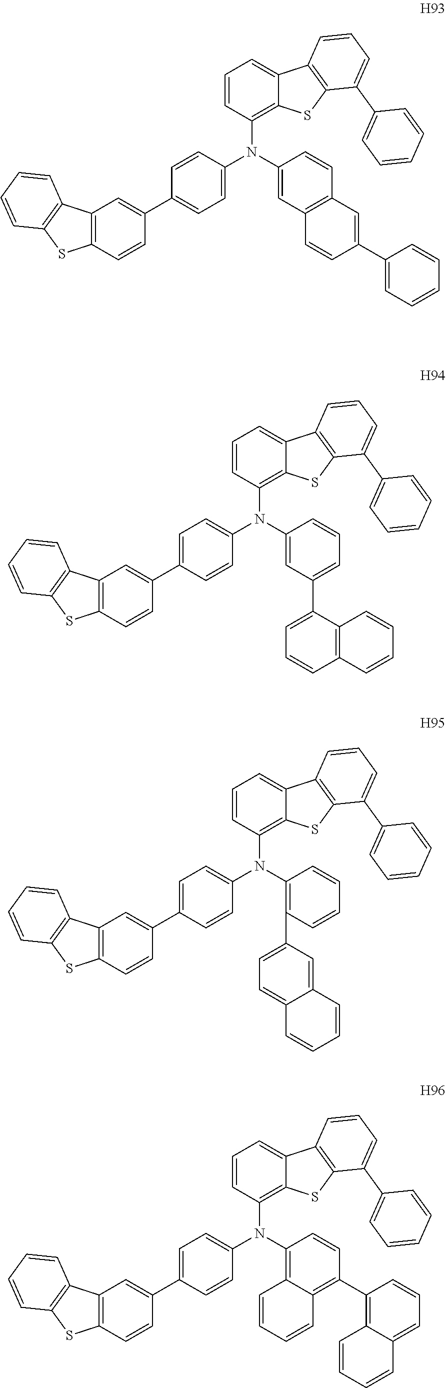 [SMILES: c1ccc(-c2ccc3cc(N(c4ccc(-c5ccc6sc7ccccc7c6c5)cc4)c4cccc5c4sc4c(-c6ccccc6)cccc45)ccc3c2)cc1 ; c1ccc(-c2cccc3c2sc2c(N(c4ccc(-c5ccc6sc7ccccc7c6c5)cc4)c4cccc(-c5cccc6ccccc56)c4)cccc23)cc1 ; c1ccc(-c2cccc3c2sc2c(N(c4ccc(-c5ccc6sc7ccccc7c6c5)cc4)c4ccccc4-c4ccc5ccccc5c4)cccc23)cc1 ; c1ccc(-c2cccc3c2sc2c(N(c4ccc(-c5ccc6sc7ccccc7c6c5)cc4)c4ccc(-c5cccc6ccccc56)c5ccccc45)cccc23)cc1]

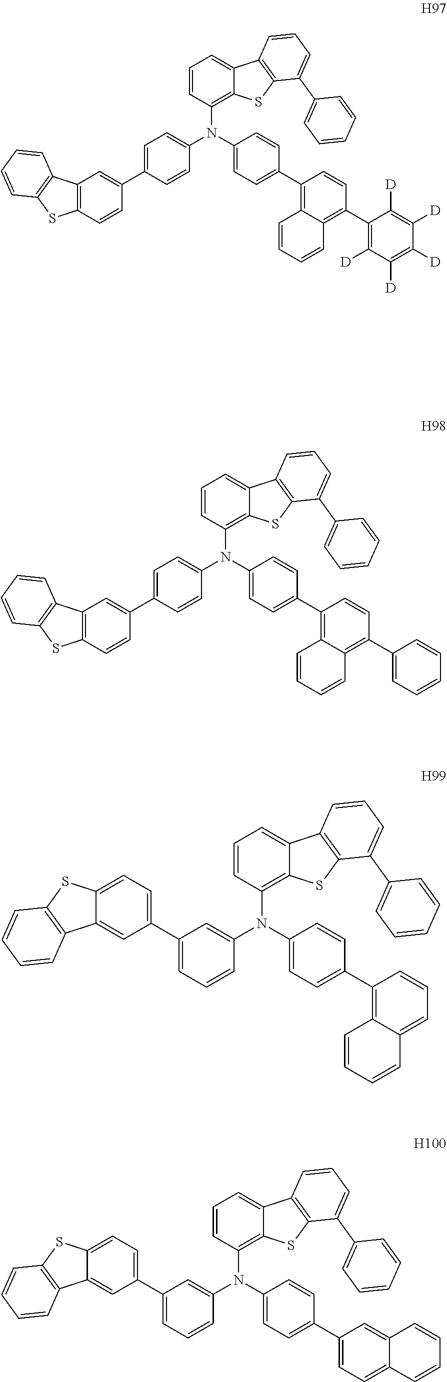 [SMILES: [2H]c1c([2H])c([2H])c(-c2ccc(-c3ccc(N(c4ccc(-c5ccc6sc7ccccc7c6c5)cc4)c4cccc5c4sc4c(-c6ccccc6)cccc45)cc3)c3ccccc23)c([2H])c1[2H] ; c1ccc(-c2ccc(-c3ccc(N(c4ccc(-c5ccc6sc7ccccc7c6c5)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4cccc(-c5ccc6sc7ccccc7c6c5)c4)cccc23)cc1 ; c1ccc(-c2cccc3c2sc2c(N(c4ccc(-c5ccc6ccccc6c5)cc4)c4cccc(-c5ccc6sc7ccccc7c6c5)c4)cccc23)cc1]

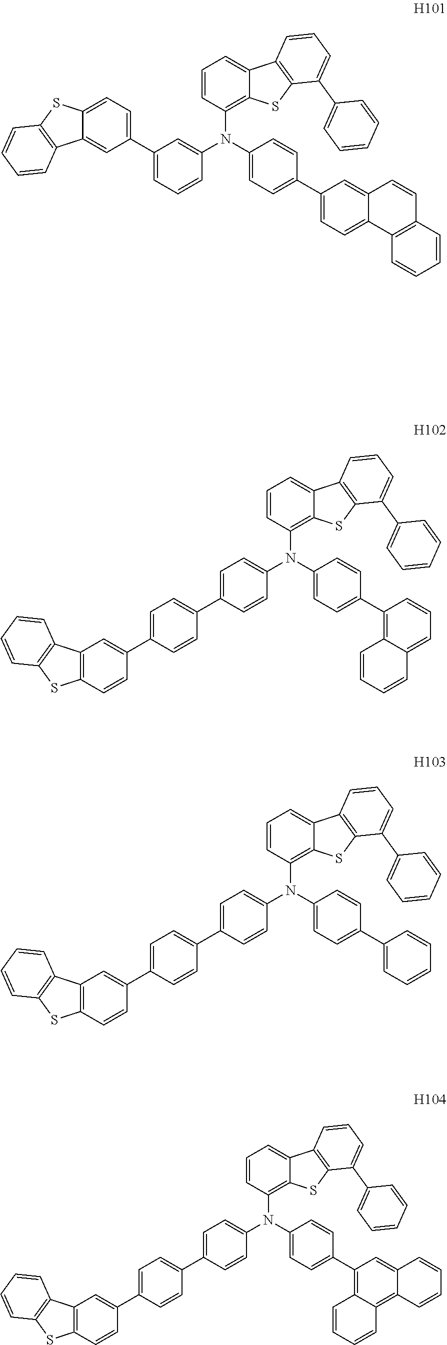 [SMILES: c1ccc(-c2cccc3c2sc2c(N(c4ccc(-c5ccc6c(ccc7ccccc76)c5)cc4)c4cccc(-c5ccc6sc7ccccc7c6c5)c4)cccc23)cc1 ; c1ccc(-c2cccc3c2sc2c(N(c4ccc(-c5ccc(-c6ccc7sc8ccccc8c7c6)cc5)cc4)c4ccc(-c5cccc6ccccc56)cc4)cccc23)cc1 ; c1ccc(-c2ccc(N(c3ccc(-c4ccc(-c5ccc6sc7ccccc7c6c5)cc4)cc3)c3cccc4c3sc3c(-c5ccccc5)cccc34)cc2)cc1 ; c1ccc(-c2cccc3c2sc2c(N(c4ccc(-c5ccc(-c6ccc7sc8ccccc8c7c6)cc5)cc4)c4ccc(-c5cc6ccccc6c6ccccc56)cc4)cccc23)cc1]

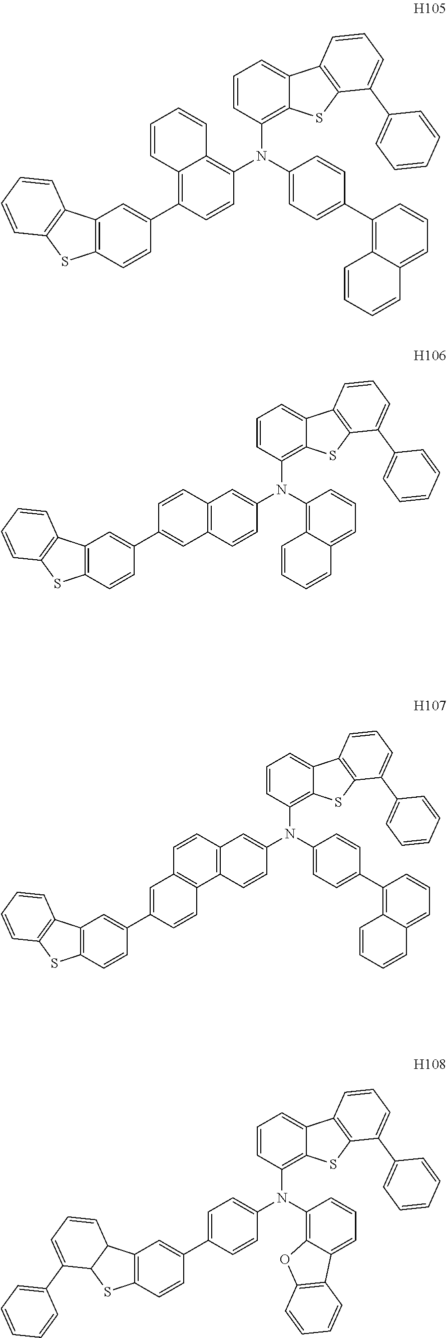 [SMILES: c1ccc(-c2cccc3c2sc2c(N(c4ccc(-c5cccc6ccccc56)cc4)c4ccc(-c5ccc6sc7ccccc7c6c5)c5ccccc45)cccc23)cc1 ; c1ccc(-c2cccc3c2sc2c(N(c4ccc5cc(-c6ccc7sc8ccccc8c7c6)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7ccc8sc9ccccc9c8c7)ccc65)c4)cccc23)cc1 ; C1=CC2c3cc(-c4ccc(N(c5cccc6c5oc5ccccc56)c5cccc6c5sc5c(-c7ccccc7)cccc56)cc4)ccc3SC2C(c2ccccc2)=C1]

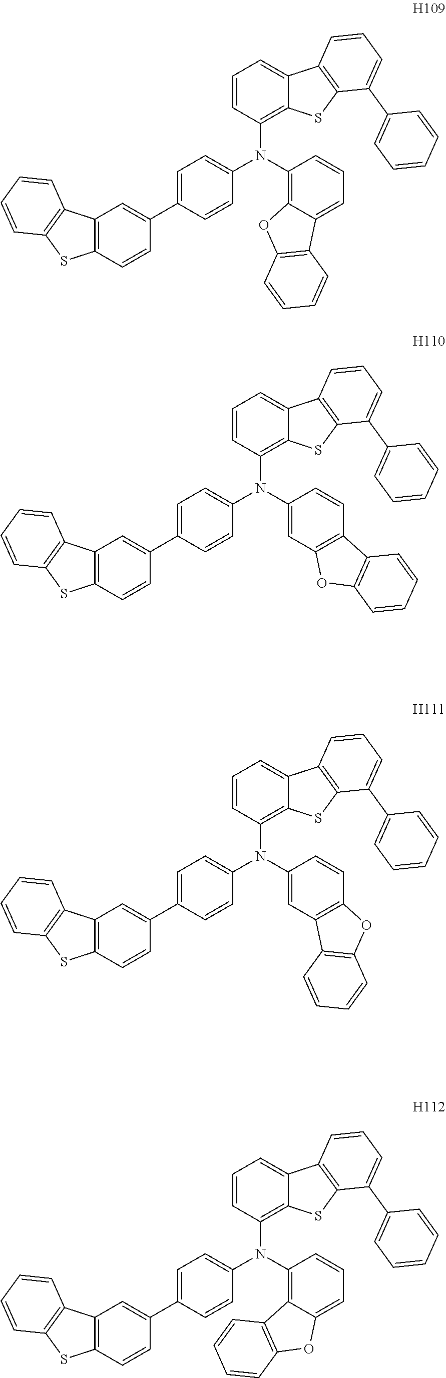 [SMILES: c1ccc(-c2cccc3c2sc2c(N(c4ccc(-c5ccc6sc7ccccc7c6c5)cc4)c4cccc5c4oc4ccccc45)cccc23)cc1 ; c1ccc(-c2cccc3c2sc2c(N(c4ccc(-c5ccc6sc7ccccc7c6c5)cc4)c4ccc5c(c4)oc4ccccc45)cccc23)cc1 ; c1ccc(-c2cccc3c2sc2c(N(c4ccc(-c5ccc6sc7ccccc7c6c5)cc4)c4ccc5oc6ccccc6c5c4)cccc23)cc1 ; c1ccc(-c2cccc3c2sc2c(N(c4ccc(-c5ccc6sc7ccccc7c6c5)cc4)c4cccc5oc6ccccc6c45)cccc23)cc1]

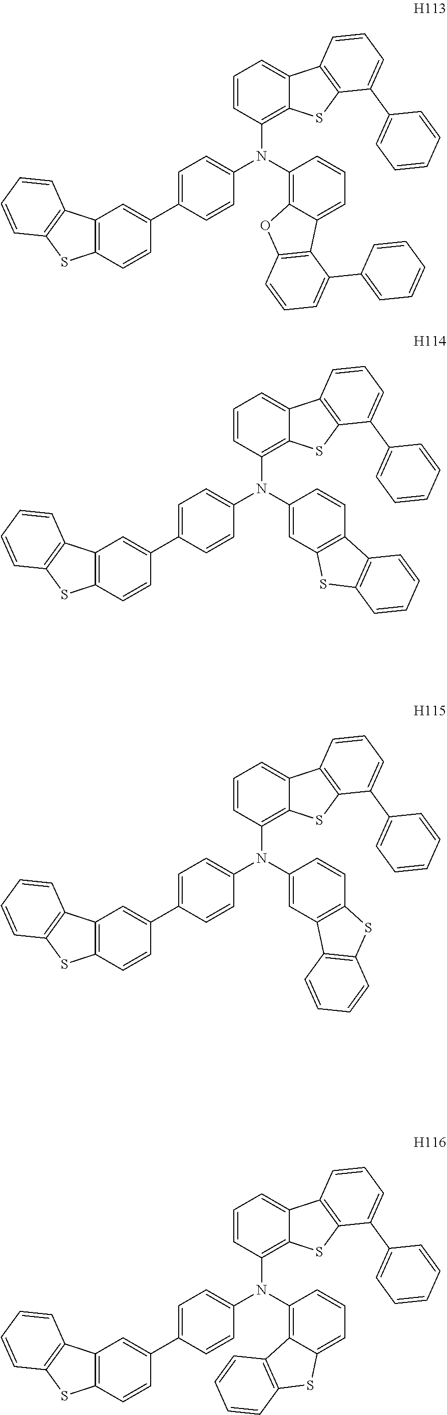 [SMILES: c1ccc(-c2cccc3c2sc2c(N(c4ccc(-c5ccc6sc7ccccc7c6c5)cc4)c4cccc5c4oc4cccc(-c6ccccc6)c45)cccc23)cc1 ; c1ccc(-c2cccc3c2sc2c(N(c4ccc(-c5ccc6sc7ccccc7c6c5)cc4)c4ccc5c(c4)sc4ccccc45)cccc23)cc1 ; c1ccc(-c2cccc3c2sc2c(N(c4ccc(-c5ccc6sc7ccccc7c6c5)cc4)c4ccc5sc6ccccc6c5c4)cccc23)cc1 ; c1ccc(-c2cccc3c2sc2c(N(c4ccc(-c5ccc6sc7ccccc7c6c5)cc4)c4cccc5sc6ccccc6c45)cccc23)cc1]

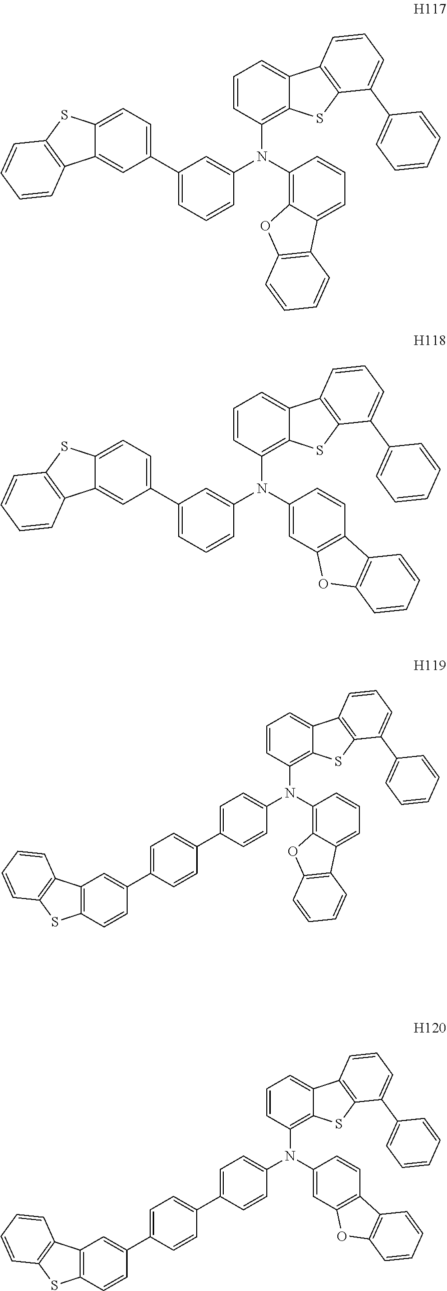 [SMILES: c1ccc(-c2cccc3c2sc2c(N(c4cccc(-c5ccc6sc7ccccc7c6c5)c4)c4cccc5c4oc4ccccc45)cccc23)cc1 ; c1ccc(-c2cccc3c2sc2c(N(c4cccc(-c5ccc6sc7ccccc7c6c5)c4)c4ccc5c(c4)oc4ccccc45)cccc23)cc1 ; c1ccc(-c2cccc3c2sc2c(N(c4ccc(-c5ccc(-c6ccc7sc8ccccc8c7c6)cc5)cc4)c4cccc5c4oc4ccccc45)cccc23)cc1 ; c1ccc(-c2cccc3c2sc2c(N(c4ccc(-c5ccc(-c6ccc7sc8ccccc8c7c6)cc5)cc4)c4ccc5c(c4)oc4ccccc45)cccc23)cc1]

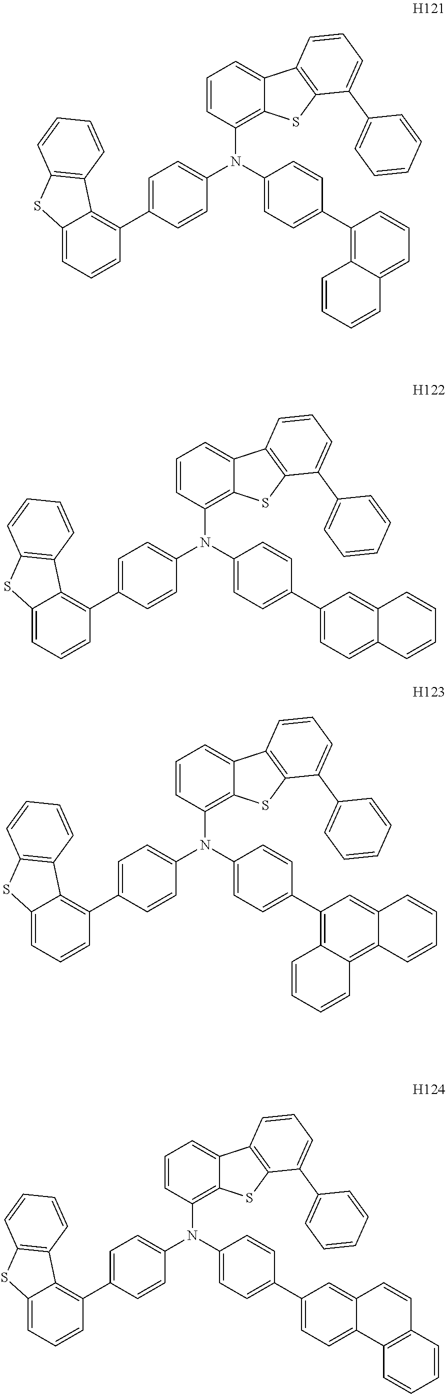 [SMILES: c1ccc(-c2cccc3c2sc2c(N(c4ccc(-c5cccc6ccccc56)cc4)c4ccc(-c5cccc6sc7ccccc7c56)cc4)cccc23)cc1 ; c1ccc(-c2cccc3c2sc2c(N(c4ccc(-c5ccc6ccccc6c5)cc4)c4ccc(-c5cccc6sc7ccccc7c56)cc4)cccc23)cc1 ; c1ccc(-c2cccc3c2sc2c(N(c4ccc(-c5cc6ccccc6c6ccccc56)cc4)c4ccc(-c5cccc6sc7ccccc7c56)cc4)cccc23)cc1 ; c1ccc(-c2cccc3c2sc2c(N(c4ccc(-c5ccc6c(ccc7ccccc76)c5)cc4)c4ccc(-c5cccc6sc7ccccc7c56)cc4)cccc23)cc1]

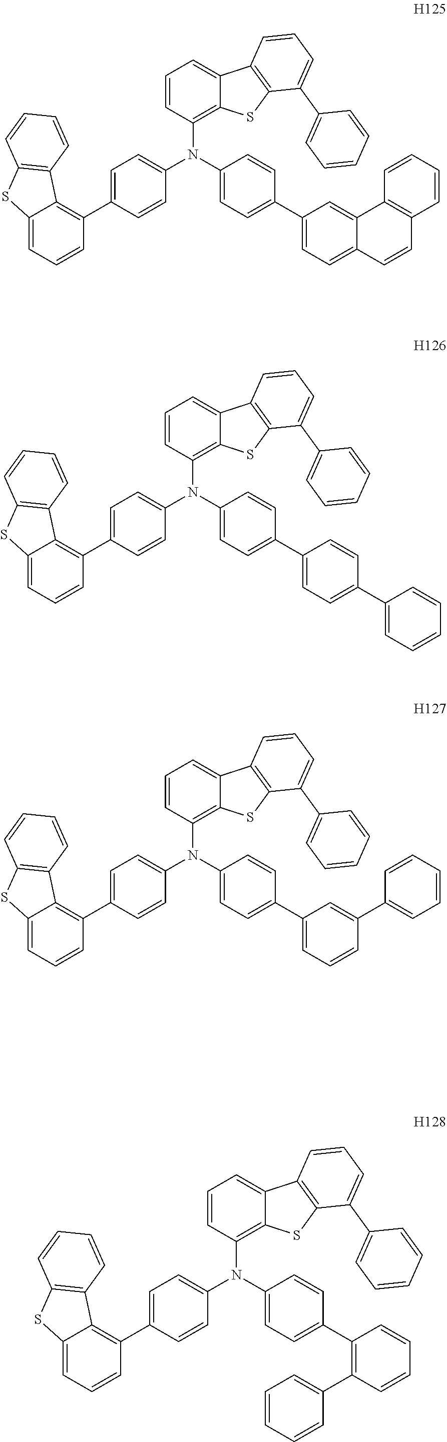 [SMILES: c1ccc(-c2cccc3c2sc2c(N(c4ccc(-c5ccc6ccc7ccccc7c6c5)cc4)c4ccc(-c5cccc6sc7ccccc7c56)cc4)cccc23)cc1 ; c1ccc(-c2ccc(-c3ccc(N(c4ccc(-c5cccc6sc7ccccc7c56)cc4)c4cccc5c4sc4c(-c6ccccc6)cccc45)cc3)cc2)cc1 ; c1ccc(-c2cccc(-c3ccc(N(c4ccc(-c5cccc6sc7ccccc7c56)cc4)c4cccc5c4sc4c(-c6ccccc6)cccc45)cc3)c2)cc1 ; c1ccc(-c2ccccc2-c2ccc(N(c3ccc(-c4cccc5sc6ccccc6c45)cc3)c3cccc4c3sc3c(-c5ccccc5)cccc34)cc2)cc1]

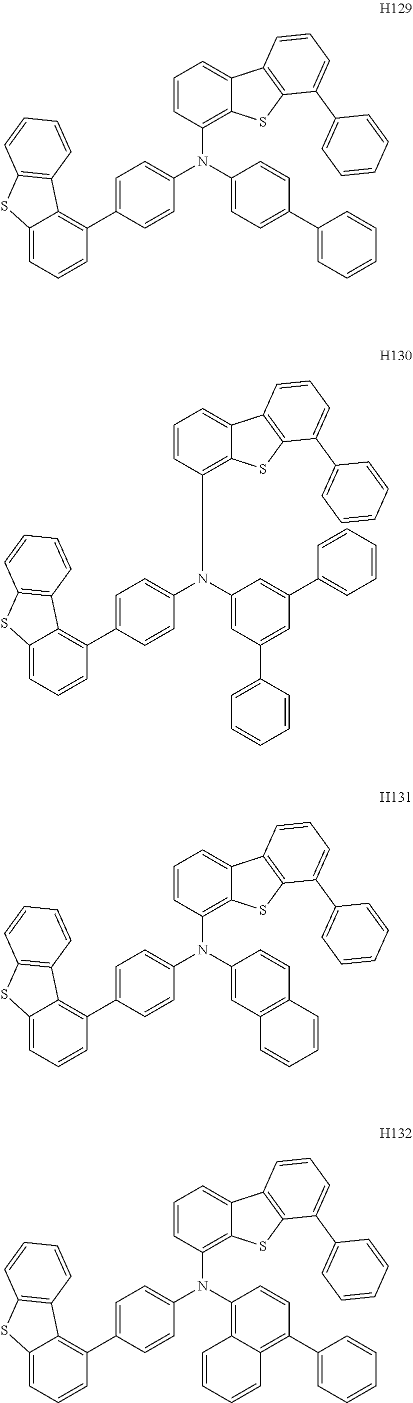 [SMILES: c1ccc(-c2ccc(N(c3ccc(-c4cccc5sc6ccccc6c45)cc3)c3cccc4c3sc3c(-c5ccccc5)cccc34)cc2)cc1 ; c1ccc(-c2cc(-c3ccccc3)cc(N(c3ccc(-c4cccc5sc6ccccc6c45)cc3)c3cccc4c3sc3c(-c5ccccc5)cccc34)c2)cc1 ; c1ccc(-c2cccc3c2sc2c(N(c4ccc(-c5cccc6sc7ccccc7c56)cc4)c4ccc5ccccc5c4)cccc23)cc1 ; c1ccc(-c2ccc(N(c3ccc(-c4cccc5sc6ccccc6c45)cc3)c3cccc4c3sc3c(-c5ccccc5)cccc34)c3ccccc23)cc1]

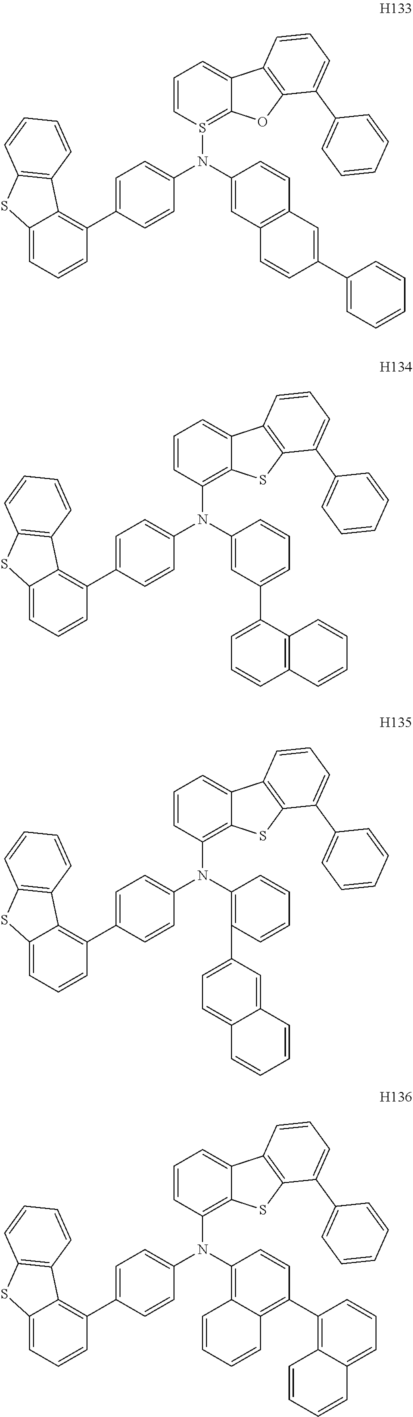 [SMILES: C1=CS(N(c2ccc(-c3cccc4sc5ccccc5c34)cc2)c2ccc3cc(-c4ccccc4)ccc3c2)=c2oc3c(-c4ccccc4)cccc3c2=C1 ; c1ccc(-c2cccc3c2sc2c(N(c4ccc(-c5cccc6sc7ccccc7c56)cc4)c4cccc(-c5cccc6ccccc56)c4)cccc23)cc1 ; c1ccc(-c2cccc3c2sc2c(N(c4ccc(-c5cccc6sc7ccccc7c56)cc4)c4ccccc4-c4ccc5ccccc5c4)cccc23)cc1 ; c1ccc(-c2cccc3c2sc2c(N(c4ccc(-c5cccc6sc7ccccc7c56)cc4)c4ccc(-c5cccc6ccccc56)c5ccccc45)cccc23)cc1]

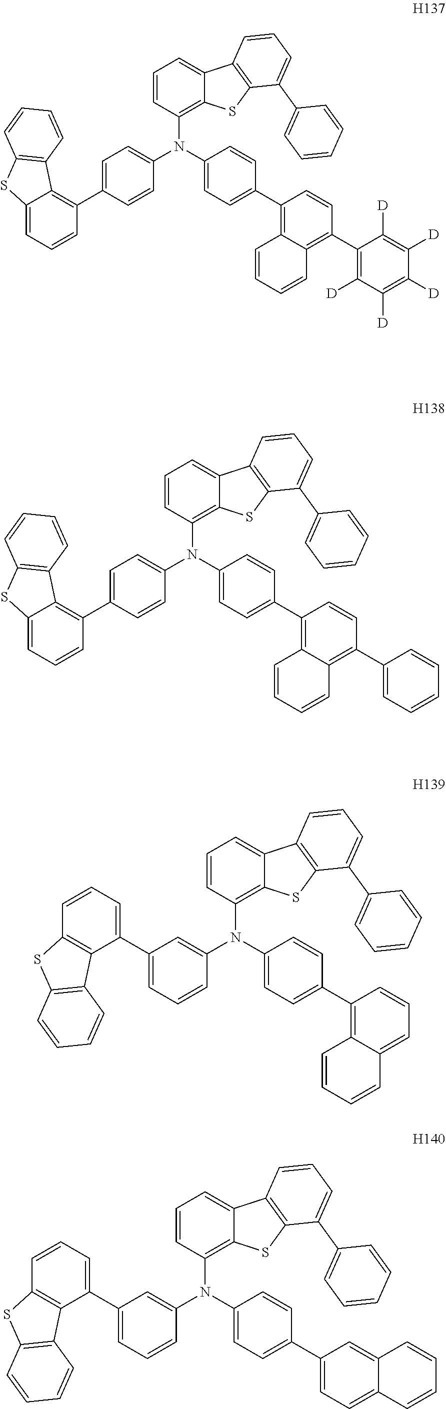 [SMILES: [2H]c1c([2H])c([2H])c(-c2ccc(-c3ccc(N(c4ccc(-c5cccc6sc7ccccc7c56)cc4)c4cccc5c4sc4c(-c6ccccc6)cccc45)cc3)c3ccccc23)c([2H])c1[2H] ; c1ccc(-c2ccc(-c3ccc(N(c4ccc(-c5cccc6sc7ccccc7c56)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4cccc(-c5cccc6sc7ccccc7c56)c4)cccc23)cc1 ; c1ccc(-c2cccc3c2sc2c(N(c4ccc(-c5ccc6ccccc6c5)cc4)c4cccc(-c5cccc6sc7ccccc7c56)c4)cccc23)cc1]

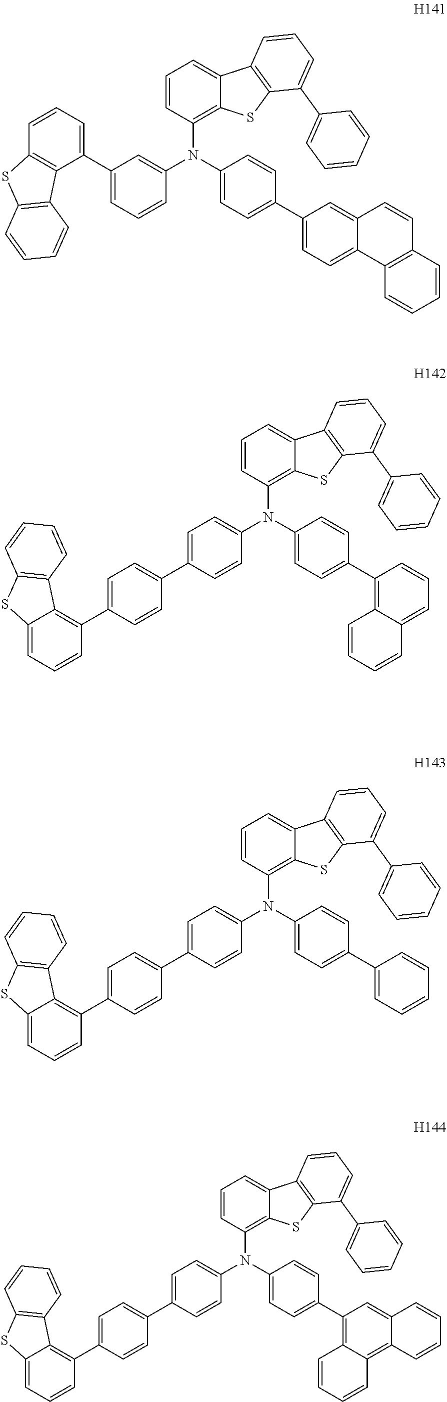 [SMILES: c1ccc(-c2cccc3c2sc2c(N(c4ccc(-c5ccc6c(ccc7ccccc76)c5)cc4)c4cccc(-c5cccc6sc7ccccc7c56)c4)cccc23)cc1 ; c1ccc(-c2cccc3c2sc2c(N(c4ccc(-c5ccc(-c6cccc7sc8ccccc8c67)cc5)cc4)c4ccc(-c5cccc6ccccc56)cc4)cccc23)cc1 ; c1ccc(-c2ccc(N(c3ccc(-c4ccc(-c5cccc6sc7ccccc7c56)cc4)cc3)c3cccc4c3sc3c(-c5ccccc5)cccc34)cc2)cc1 ; c1ccc(-c2cccc3c2sc2c(N(c4ccc(-c5ccc(-c6cccc7sc8ccccc8c67)cc5)cc4)c4ccc(-c5cc6ccccc6c6ccccc56)cc4)cccc23)cc1]

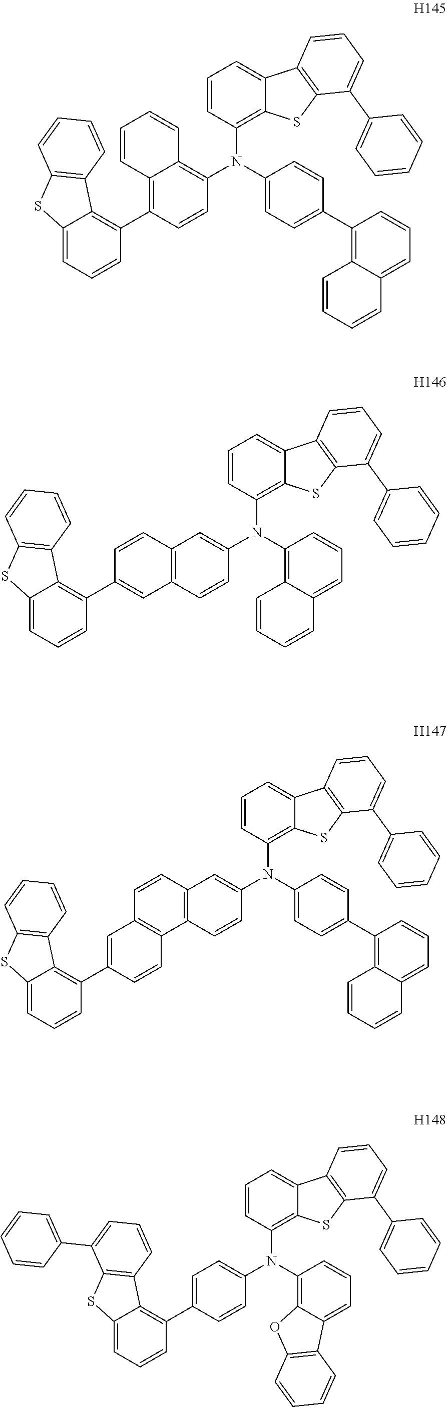 [SMILES: c1ccc(-c2cccc3c2sc2c(N(c4ccc(-c5cccc6ccccc56)cc4)c4ccc(-c5cccc6sc7ccccc7c56)c5ccccc45)cccc23)cc1 ; c1ccc(-c2cccc3c2sc2c(N(c4ccc5cc(-c6cccc7sc8ccccc8c67)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7cccc8sc9ccccc9c78)ccc65)c4)cccc23)cc1 ; c1ccc(-c2cccc3c2sc2c(N(c4ccc(-c5cccc6sc7c(-c8ccccc8)cccc7c56)cc4)c4cccc5c4oc4ccccc45)cccc23)cc1]

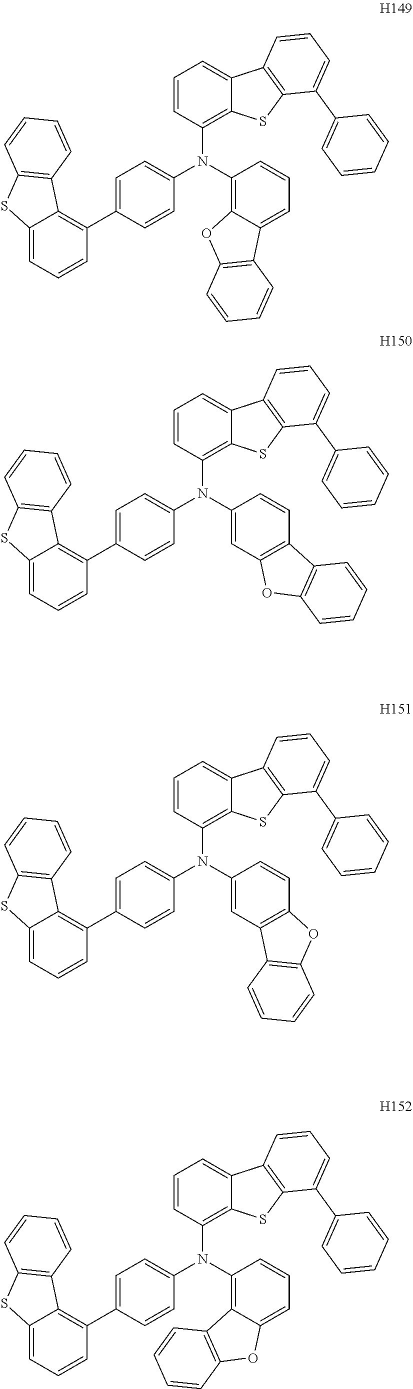 [SMILES: c1ccc(-c2cccc3c2sc2c(N(c4ccc(-c5cccc6sc7ccccc7c56)cc4)c4cccc5c4oc4ccccc45)cccc23)cc1 ; c1ccc(-c2cccc3c2sc2c(N(c4ccc(-c5cccc6sc7ccccc7c56)cc4)c4ccc5c(c4)oc4ccccc45)cccc23)cc1 ; c1ccc(-c2cccc3c2sc2c(N(c4ccc(-c5cccc6sc7ccccc7c56)cc4)c4ccc5oc6ccccc6c5c4)cccc23)cc1 ; c1ccc(-c2cccc3c2sc2c(N(c4ccc(-c5cccc6sc7ccccc7c56)cc4)c4cccc5oc6ccccc6c45)cccc23)cc1]

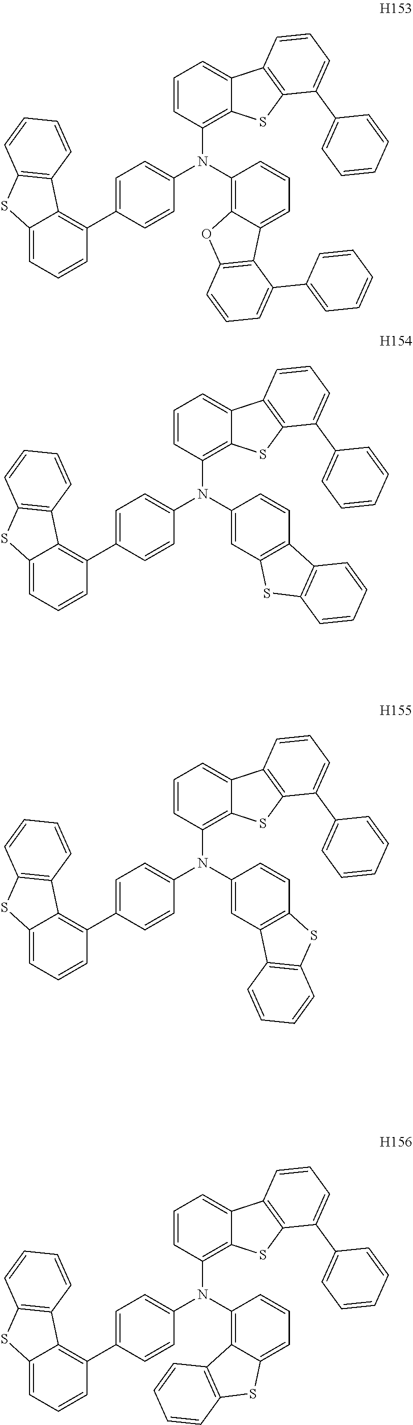 [SMILES: c1ccc(-c2cccc3c2sc2c(N(c4ccc(-c5cccc6sc7ccccc7c56)cc4)c4cccc5c4oc4cccc(-c6ccccc6)c45)cccc23)cc1 ; c1ccc(-c2cccc3c2sc2c(N(c4ccc(-c5cccc6sc7ccccc7c56)cc4)c4ccc5c(c4)sc4ccccc45)cccc23)cc1 ; c1ccc(-c2cccc3c2sc2c(N(c4ccc(-c5cccc6sc7ccccc7c56)cc4)c4ccc5sc6ccccc6c5c4)cccc23)cc1 ; c1ccc(-c2cccc3c2sc2c(N(c4ccc(-c5cccc6sc7ccccc7c56)cc4)c4cccc5sc6ccccc6c45)cccc23)cc1]

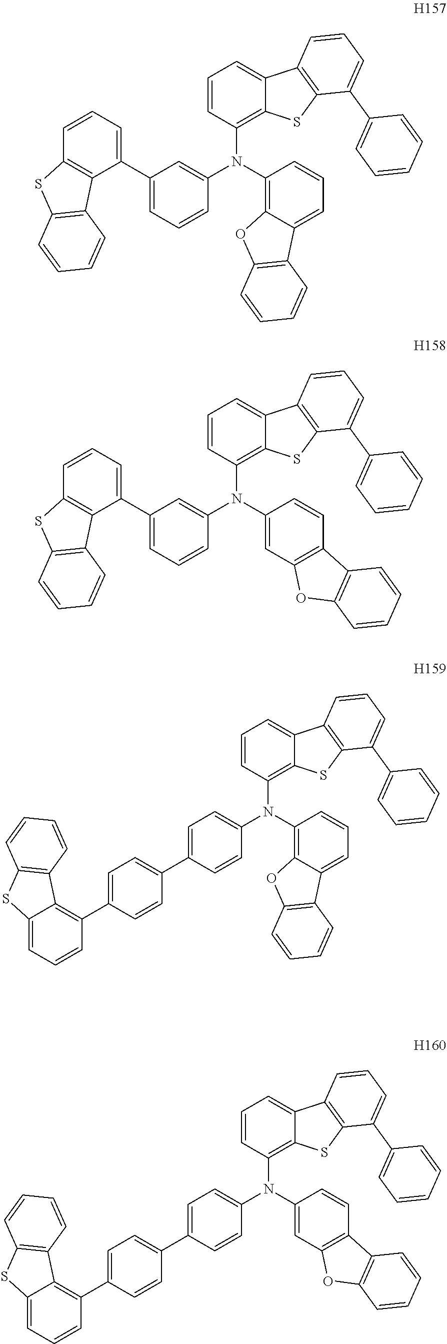 [SMILES: c1ccc(-c2cccc3c2sc2c(N(c4cccc(-c5cccc6sc7ccccc7c56)c4)c4cccc5c4oc4ccccc45)cccc23)cc1 ; c1ccc(-c2cccc3c2sc2c(N(c4cccc(-c5cccc6sc7ccccc7c56)c4)c4ccc5c(c4)oc4ccccc45)cccc23)cc1 ; c1ccc(-c2cccc3c2sc2c(N(c4ccc(-c5ccc(-c6cccc7sc8ccccc8c67)cc5)cc4)c4cccc5c4oc4ccccc45)cccc23)cc1 ; c1ccc(-c2cccc3c2sc2c(N(c4ccc(-c5ccc(-c6cccc7sc8ccccc8c67)cc5)cc4)c4ccc5c(c4)oc4ccccc45)cccc23)cc1]

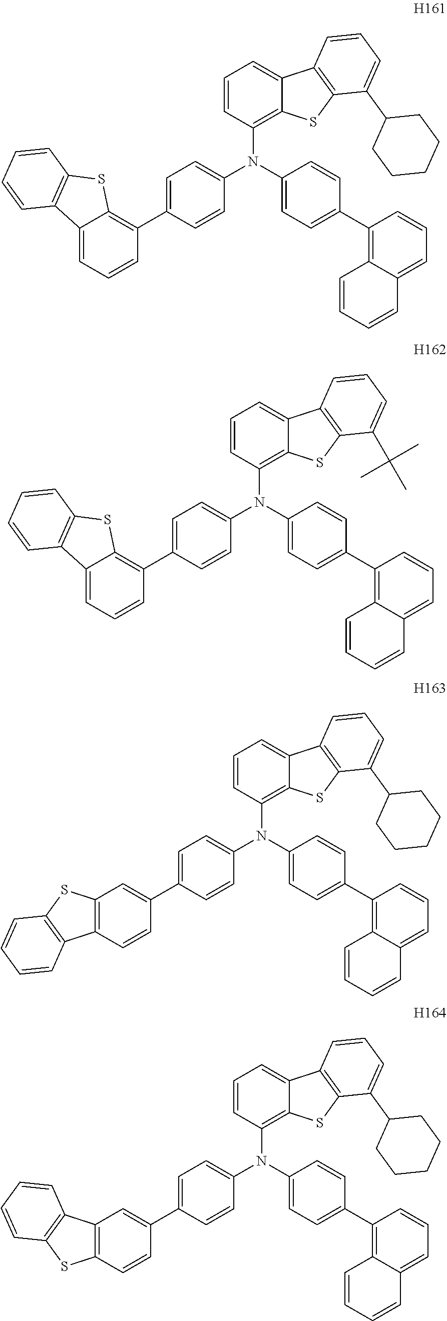

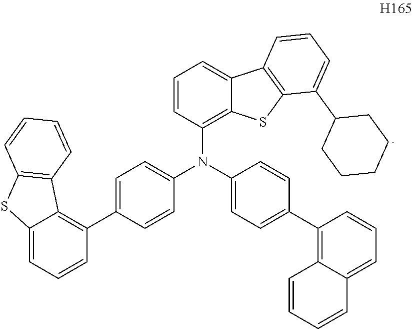 [SMILES: c1ccc2c(-c3ccc(N(c4ccc(-c5cccc6sc7ccccc7c56)cc4)c4cccc5c4sc4c(C6CCCCC6)cccc45)cc3)cccc2c1]

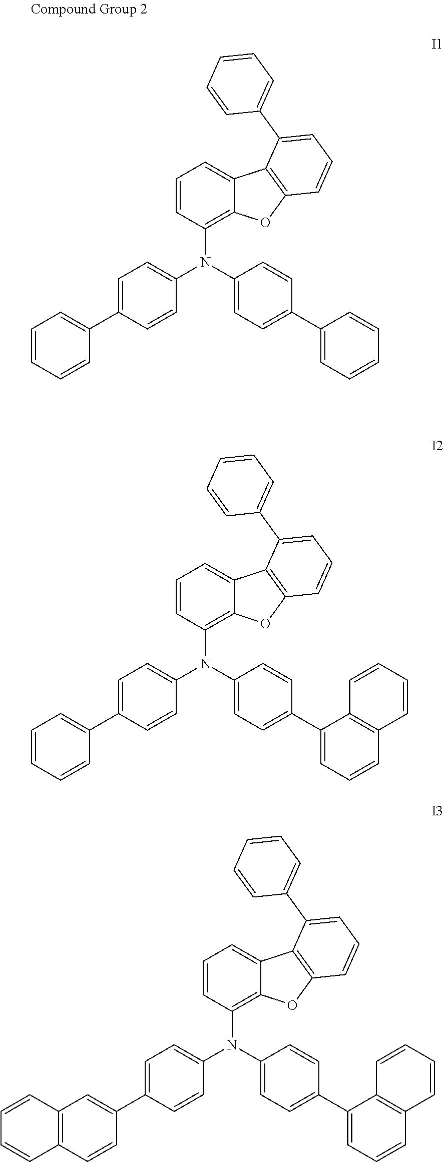 [SMILES: c1ccc(-c2ccc(N(c3ccc(-c4ccccc4)cc3)c3cccc4c3oc3cccc(-c5ccccc5)c34)cc2)cc1 ; c1ccc(-c2ccc(N(c3ccc(-c4cccc5ccccc45)cc3)c3cccc4c3oc3cccc(-c5ccccc5)c34)cc2)cc1 ; c1ccc(-c2cccc3oc4c(N(c5ccc(-c6ccc7ccccc7c6)cc5)c5ccc(-c6cccc7ccccc67)cc5)cccc4c23)cc1]

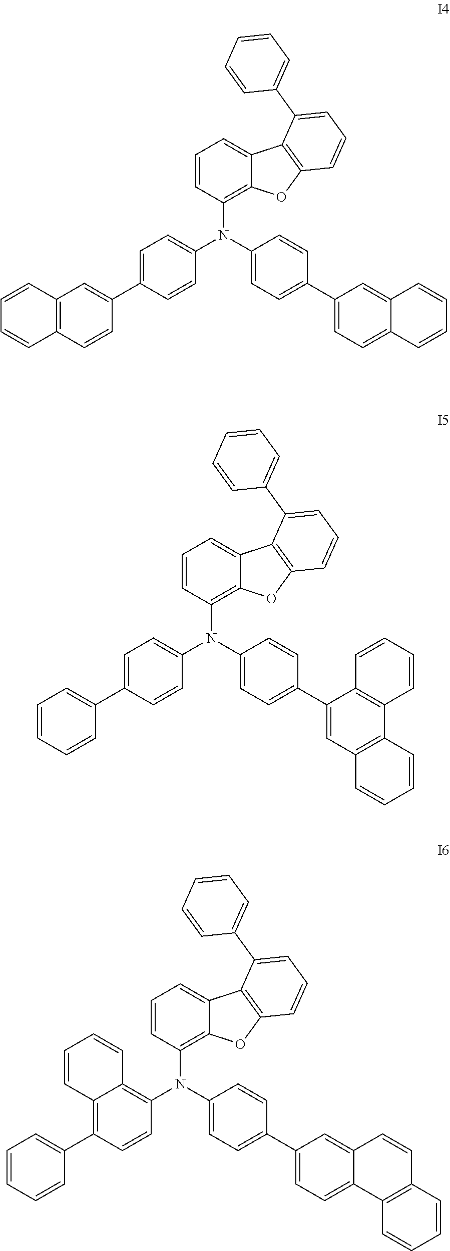 [SMILES: c1ccc(-c2cccc3oc4c(N(c5ccc(-c6ccc7ccccc7c6)cc5)c5ccc(-c6ccc7ccccc7c6)cc5)cccc4c23)cc1 ; c1ccc(-c2ccc(N(c3ccc(-c4cc5ccccc5c5ccccc45)cc3)c3cccc4c3oc3cccc(-c5ccccc5)c34)cc2)cc1 ; c1ccc(-c2ccc(N(c3ccc(-c4ccc5c(ccc6ccccc65)c4)cc3)c3cccc4c3oc3cccc(-c5ccccc5)c34)c3ccccc23)cc1]

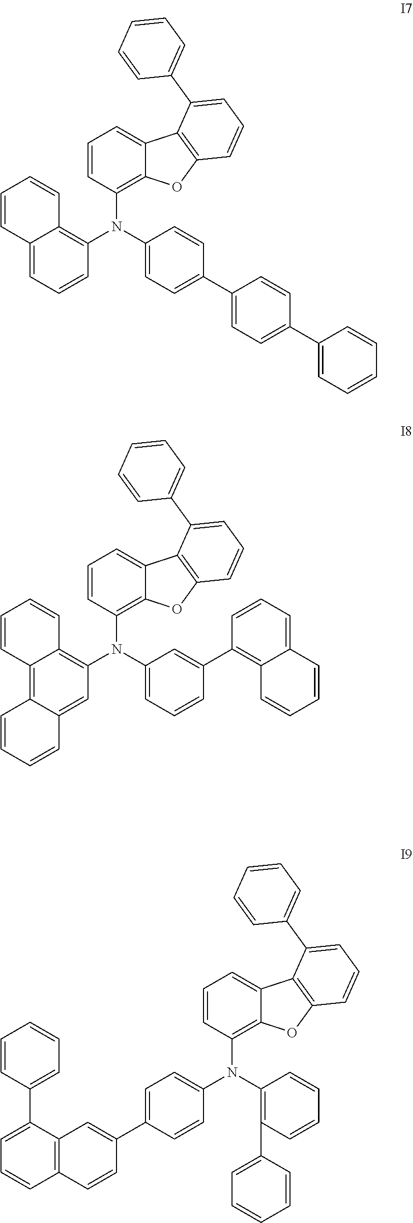 [SMILES: c1ccc(-c2ccc(-c3ccc(N(c4cccc5ccccc45)c4cccc5c4oc4cccc(-c6ccccc6)c45)cc3)cc2)cc1 ; c1ccc(-c2cccc3oc4c(N(c5cccc(-c6cccc7ccccc67)c5)c5cc6ccccc6c6ccccc56)cccc4c23)cc1 ; c1ccc(-c2ccccc2N(c2ccc(-c3ccc4cccc(-c5ccccc5)c4c3)cc2)c2cccc3c2oc2cccc(-c4ccccc4)c23)cc1]

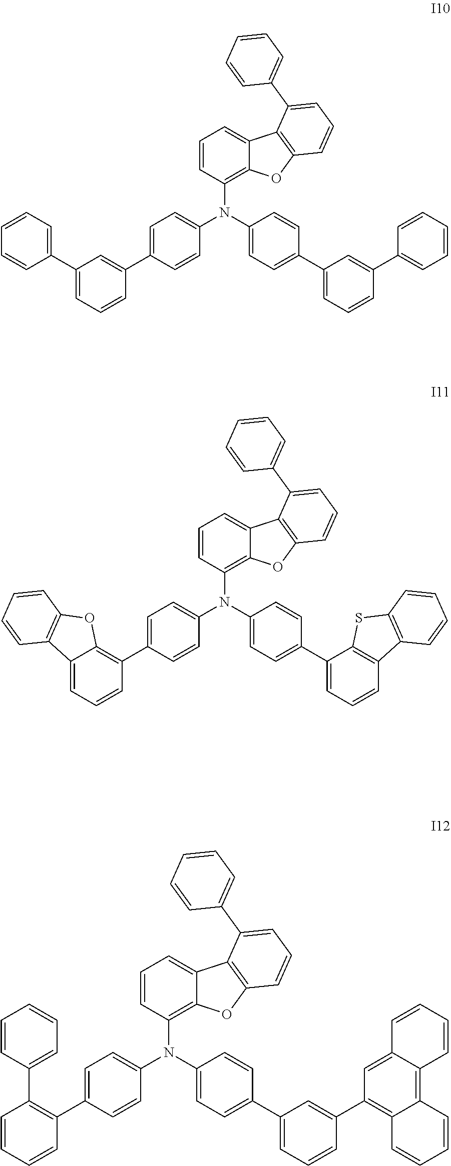 [SMILES: c1ccc(-c2cccc(-c3ccc(N(c4ccc(-c5cccc(-c6ccccc6)c5)cc4)c4cccc5c4oc4cccc(-c6ccccc6)c45)cc3)c2)cc1 ; c1ccc(-c2cccc3oc4c(N(c5ccc(-c6cccc7c6oc6ccccc67)cc5)c5ccc(-c6cccc7c6sc6ccccc67)cc5)cccc4c23)cc1 ; c1ccc(-c2ccccc2-c2ccc(N(c3ccc(-c4cccc(-c5cc6ccccc6c6ccccc56)c4)cc3)c3cccc4c3oc3cccc(-c5ccccc5)c34)cc2)cc1]

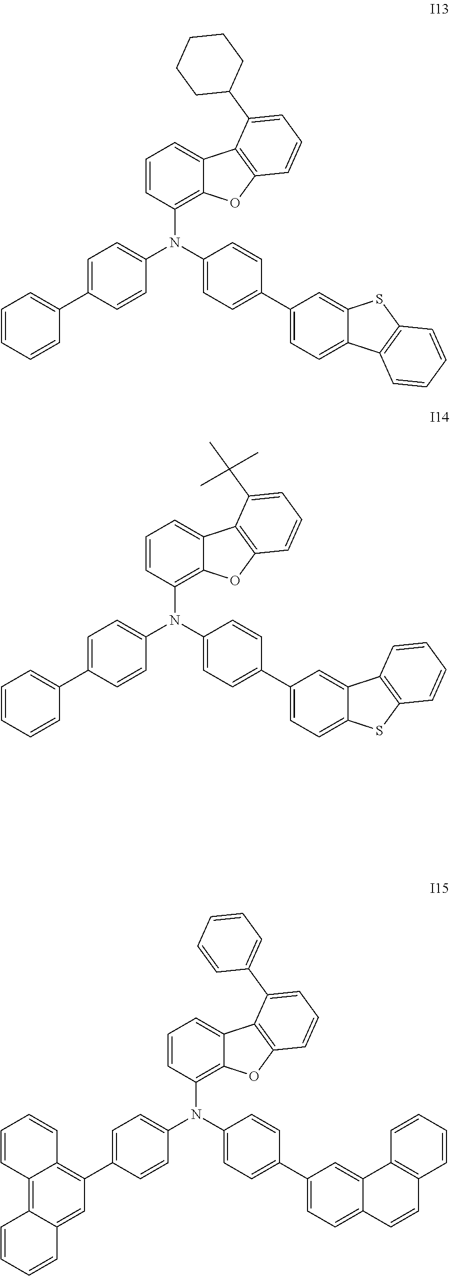 [SMILES: c1ccc(-c2ccc(N(c3ccc(-c4ccc5c(c4)sc4ccccc45)cc3)c3cccc4c3oc3cccc(C5CCCCC5)c34)cc2)cc1 ; CC(C)(C)c1cccc2oc3c(N(c4ccc(-c5ccccc5)cc4)c4ccc(-c5ccc6sc7ccccc7c6c5)cc4)cccc3c12 ; c1ccc(-c2cccc3oc4c(N(c5ccc(-c6ccc7ccc8ccccc8c7c6)cc5)c5ccc(-c6cc7ccccc7c7ccccc67)cc5)cccc4c23)cc1]

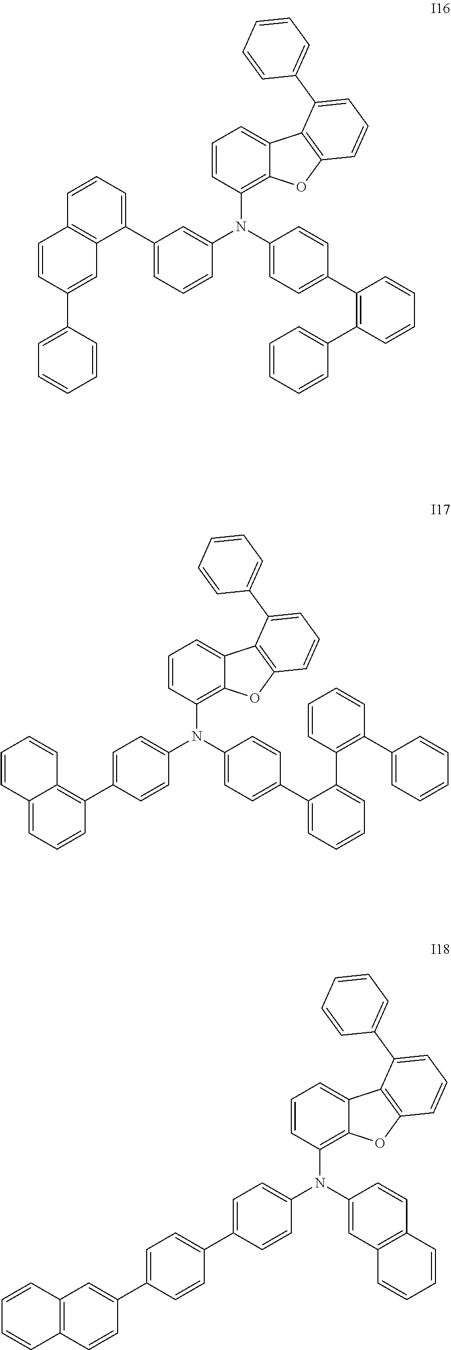 [SMILES: c1ccc(-c2ccc3cccc(-c4cccc(N(c5ccc(-c6ccccc6-c6ccccc6)cc5)c5cccc6c5oc5cccc(-c7ccccc7)c56)c4)c3c2)cc1 ; c1ccc(-c2ccccc2-c2ccccc2-c2ccc(N(c3ccc(-c4cccc5ccccc45)cc3)c3cccc4c3oc3cccc(-c5ccccc5)c34)cc2)cc1 ; c1ccc(-c2cccc3oc4c(N(c5ccc(-c6ccc(-c7ccc8ccccc8c7)cc6)cc5)c5ccc6ccccc6c5)cccc4c23)cc1]

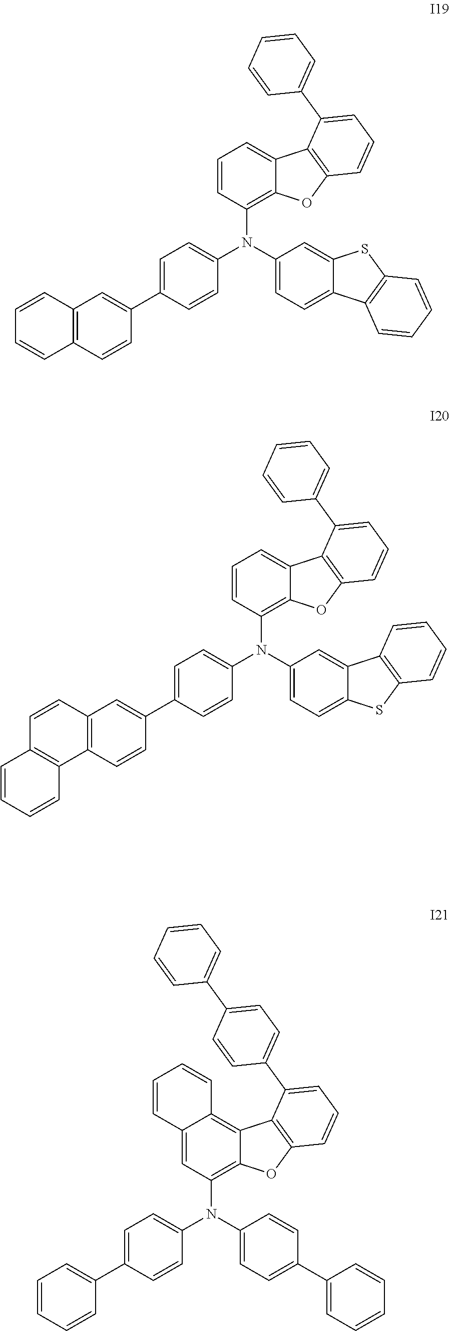 [SMILES: c1ccc(-c2cccc3oc4c(N(c5ccc(-c6ccc7ccccc7c6)cc5)c5ccc6c(c5)sc5ccccc56)cccc4c23)cc1 ; c1ccc(-c2cccc3oc4c(N(c5ccc(-c6ccc7c(ccc8ccccc87)c6)cc5)c5ccc6sc7ccccc7c6c5)cccc4c23)cc1 ; c1ccc(-c2ccc(-c3cccc4oc5c(N(c6ccc(-c7ccccc7)cc6)c6ccc(-c7ccccc7)cc6)cc6ccccc6c5c34)cc2)cc1]

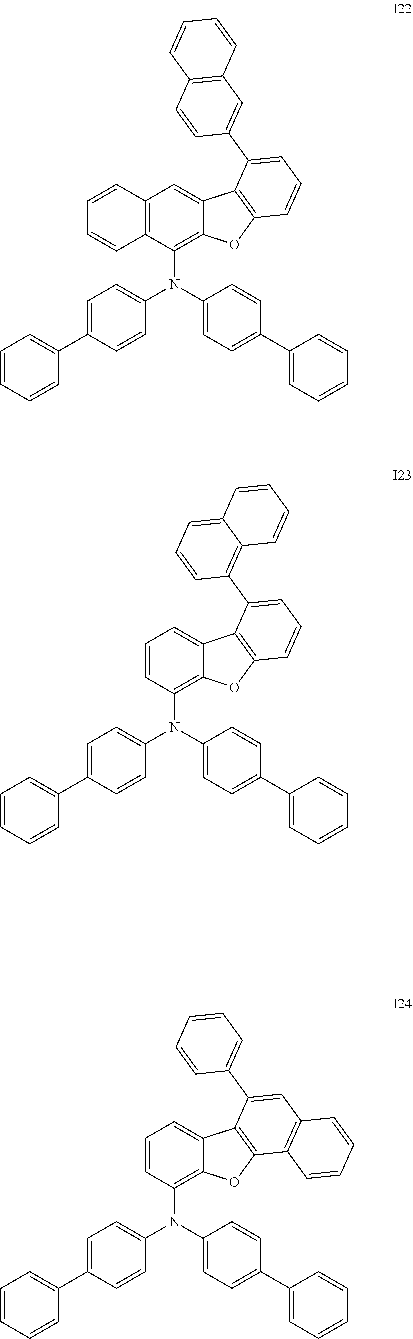 [SMILES: c1ccc(-c2ccc(N(c3ccc(-c4ccccc4)cc3)c3c4ccccc4cc4c3oc3cccc(-c5ccc6ccccc6c5)c34)cc2)cc1 ; c1ccc(-c2ccc(N(c3ccc(-c4ccccc4)cc3)c3cccc4c3oc3cccc(-c5cccc6ccccc56)c34)cc2)cc1 ; c1ccc(-c2ccc(N(c3ccc(-c4ccccc4)cc3)c3cccc4c3oc3c5ccccc5cc(-c5ccccc5)c43)cc2)cc1]

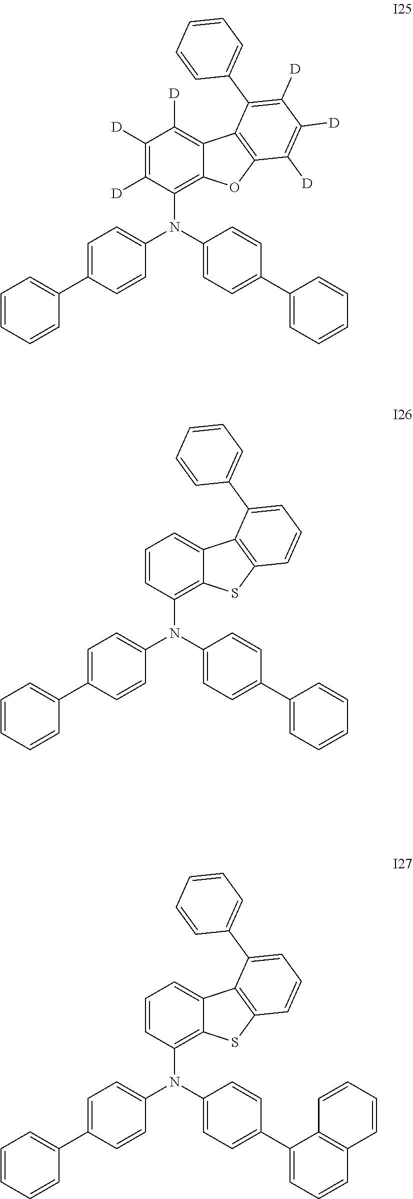 [SMILES: [2H]c1c([2H])c(-c2ccccc2)c2c(oc3c(N(c4ccc(-c5ccccc5)cc4)c4ccc(-c5ccccc5)cc4)c([2H])c([2H])c([2H])c32)c1[2H] ; c1ccc(-c2ccc(N(c3ccc(-c4ccccc4)cc3)c3cccc4c3sc3cccc(-c5ccccc5)c34)cc2)cc1 ; c1ccc(-c2ccc(N(c3ccc(-c4cccc5ccccc45)cc3)c3cccc4c3sc3cccc(-c5ccccc5)c34)cc2)cc1]

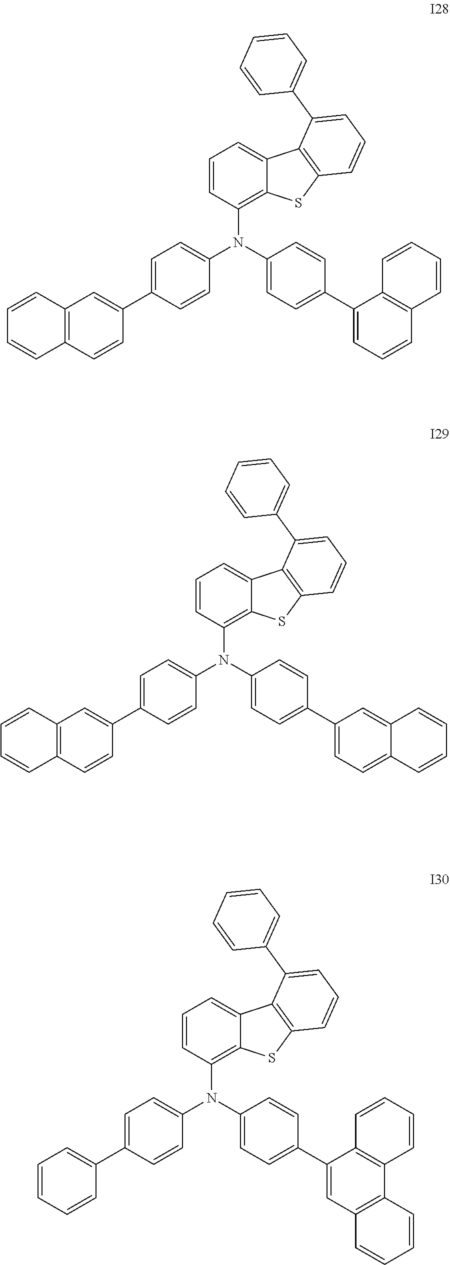 [SMILES: c1ccc(-c2cccc3sc4c(N(c5ccc(-c6ccc7ccccc7c6)cc5)c5ccc(-c6cccc7ccccc67)cc5)cccc4c23)cc1 ; c1ccc(-c2cccc3sc4c(N(c5ccc(-c6ccc7ccccc7c6)cc5)c5ccc(-c6ccc7ccccc7c6)cc5)cccc4c23)cc1 ; c1ccc(-c2ccc(N(c3ccc(-c4cc5ccccc5c5ccccc45)cc3)c3cccc4c3sc3cccc(-c5ccccc5)c34)cc2)cc1]

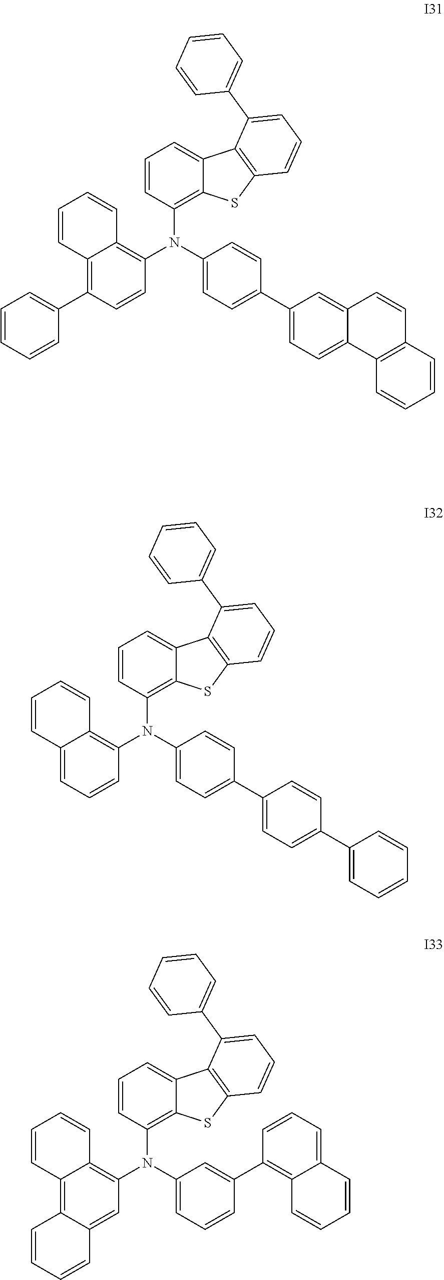 [SMILES: c1ccc(-c2ccc(N(c3ccc(-c4ccc5c(ccc6ccccc65)c4)cc3)c3cccc4c3sc3cccc(-c5ccccc5)c34)c3ccccc23)cc1 ; c1ccc(-c2ccc(-c3ccc(N(c4cccc5ccccc45)c4cccc5c4sc4cccc(-c6ccccc6)c45)cc3)cc2)cc1 ; c1ccc(-c2cccc3sc4c(N(c5cccc(-c6cccc7ccccc67)c5)c5cc6ccccc6c6ccccc56)cccc4c23)cc1]

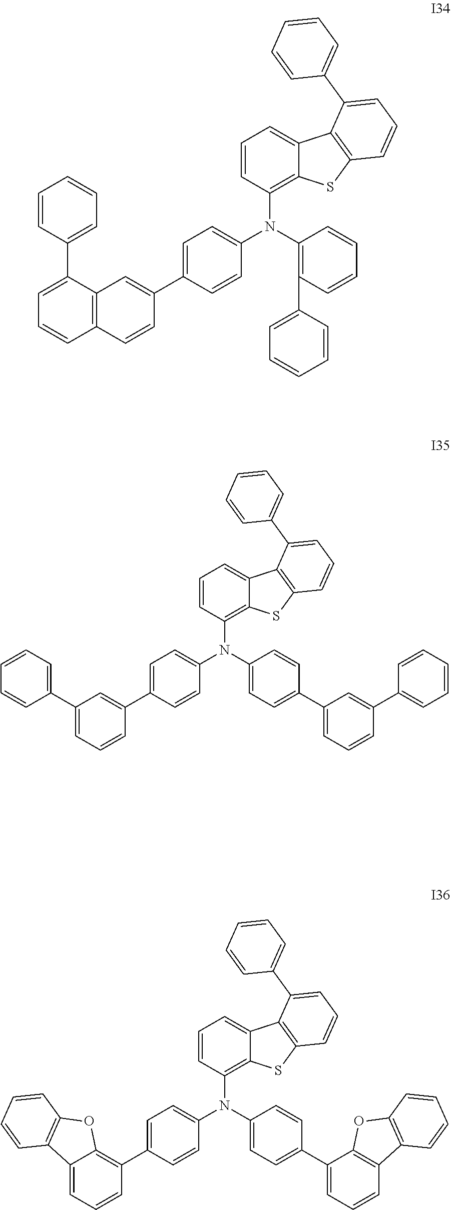 [SMILES: c1ccc(-c2ccccc2N(c2ccc(-c3ccc4cccc(-c5ccccc5)c4c3)cc2)c2cccc3c2sc2cccc(-c4ccccc4)c23)cc1 ; c1ccc(-c2cccc(-c3ccc(N(c4ccc(-c5cccc(-c6ccccc6)c5)cc4)c4cccc5c4sc4cccc(-c6ccccc6)c45)cc3)c2)cc1 ; c1ccc(-c2cccc3sc4c(N(c5ccc(-c6cccc7c6oc6ccccc67)cc5)c5ccc(-c6cccc7c6oc6ccccc67)cc5)cccc4c23)cc1]

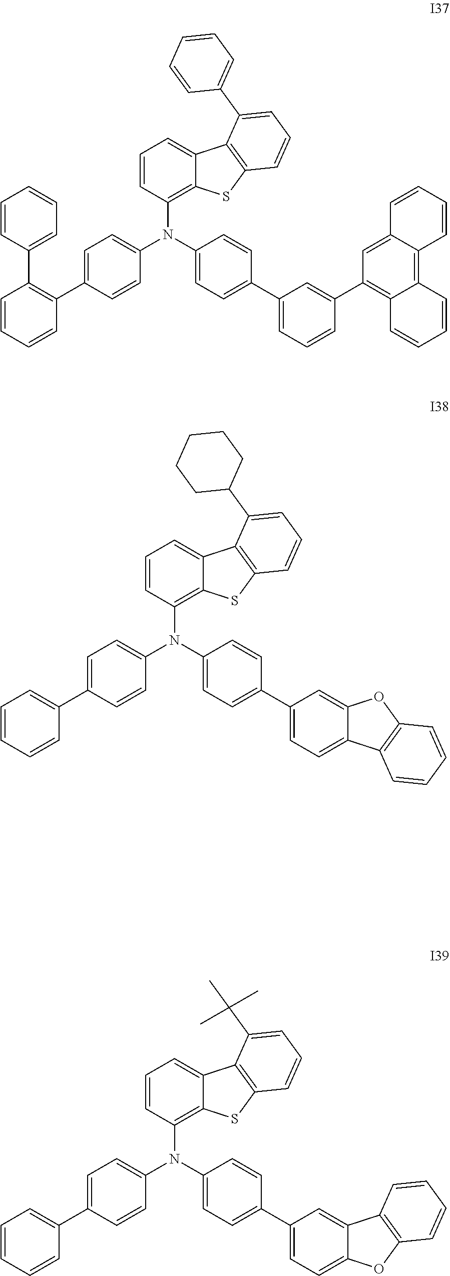 [SMILES: c1ccc(-c2ccccc2-c2ccc(N(c3ccc(-c4cccc(-c5cc6ccccc6c6ccccc56)c4)cc3)c3cccc4c3sc3cccc(-c5ccccc5)c34)cc2)cc1 ; c1ccc(-c2ccc(N(c3ccc(-c4ccc5c(c4)oc4ccccc45)cc3)c3cccc4c3sc3cccc(C5CCCCC5)c34)cc2)cc1 ; CC(C)(C)c1cccc2sc3c(N(c4ccc(-c5ccccc5)cc4)c4ccc(-c5ccc6oc7ccccc7c6c5)cc4)cccc3c12]

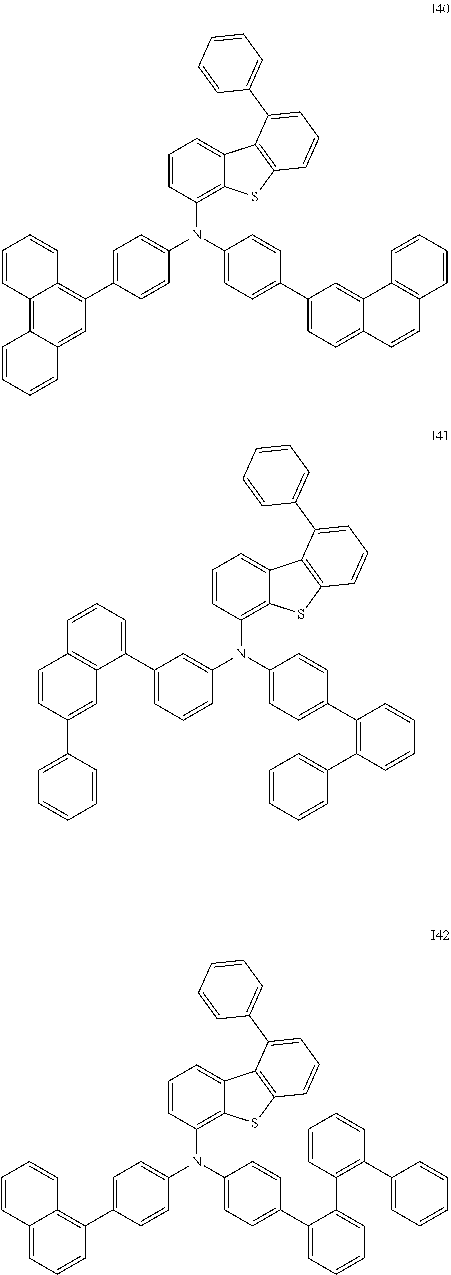 [SMILES: c1ccc(-c2cccc3sc4c(N(c5ccc(-c6ccc7ccc8ccccc8c7c6)cc5)c5ccc(-c6cc7ccccc7c7ccccc67)cc5)cccc4c23)cc1 ; c1ccc(-c2ccc3cccc(-c4cccc(N(c5ccc(-c6ccccc6-c6ccccc6)cc5)c5cccc6c5sc5cccc(-c7ccccc7)c56)c4)c3c2)cc1 ; c1ccc(-c2ccccc2-c2ccccc2-c2ccc(N(c3ccc(-c4cccc5ccccc45)cc3)c3cccc4c3sc3cccc(-c5ccccc5)c34)cc2)cc1]

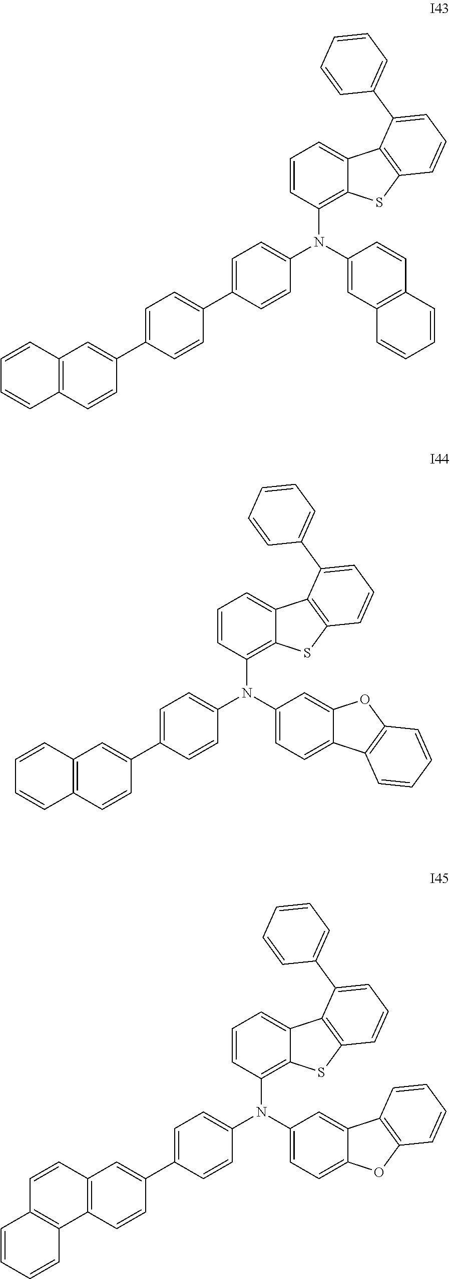 [SMILES: c1ccc(-c2cccc3sc4c(N(c5ccc(-c6ccc(-c7ccc8ccccc8c7)cc6)cc5)c5ccc6ccccc6c5)cccc4c23)cc1 ; c1ccc(-c2cccc3sc4c(N(c5ccc(-c6ccc7ccccc7c6)cc5)c5ccc6c(c5)oc5ccccc56)cccc4c23)cc1 ; c1ccc(-c2cccc3sc4c(N(c5ccc(-c6ccc7c(ccc8ccccc87)c6)cc5)c5ccc6oc7ccccc7c6c5)cccc4c23)cc1]

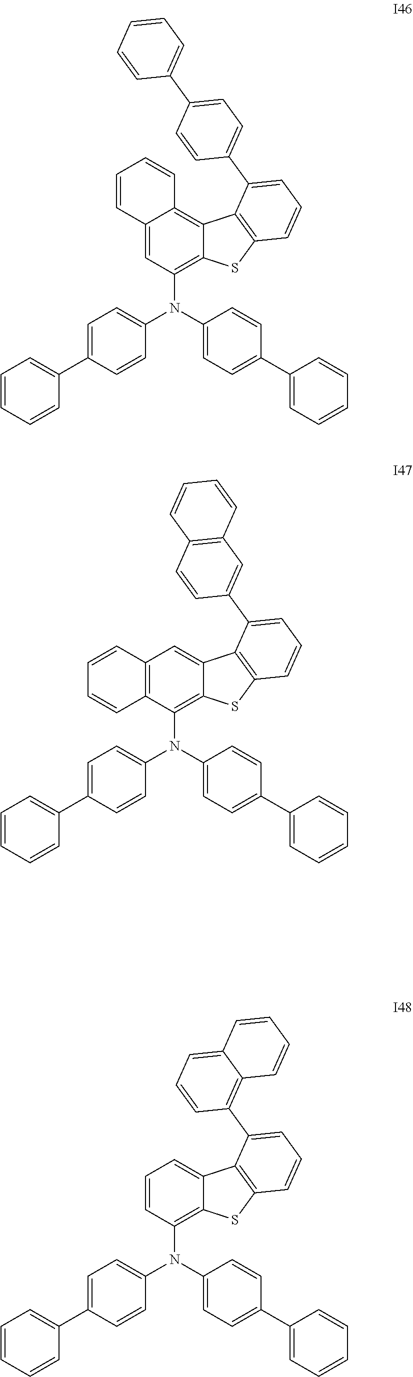 [SMILES: c1ccc(-c2ccc(-c3cccc4sc5c(N(c6ccc(-c7ccccc7)cc6)c6ccc(-c7ccccc7)cc6)cc6ccccc6c5c34)cc2)cc1 ; c1ccc(-c2ccc(N(c3ccc(-c4ccccc4)cc3)c3c4ccccc4cc4c3sc3cccc(-c5ccc6ccccc6c5)c34)cc2)cc1 ; c1ccc(-c2ccc(N(c3ccc(-c4ccccc4)cc3)c3cccc4c3sc3cccc(-c5cccc6ccccc56)c34)cc2)cc1]

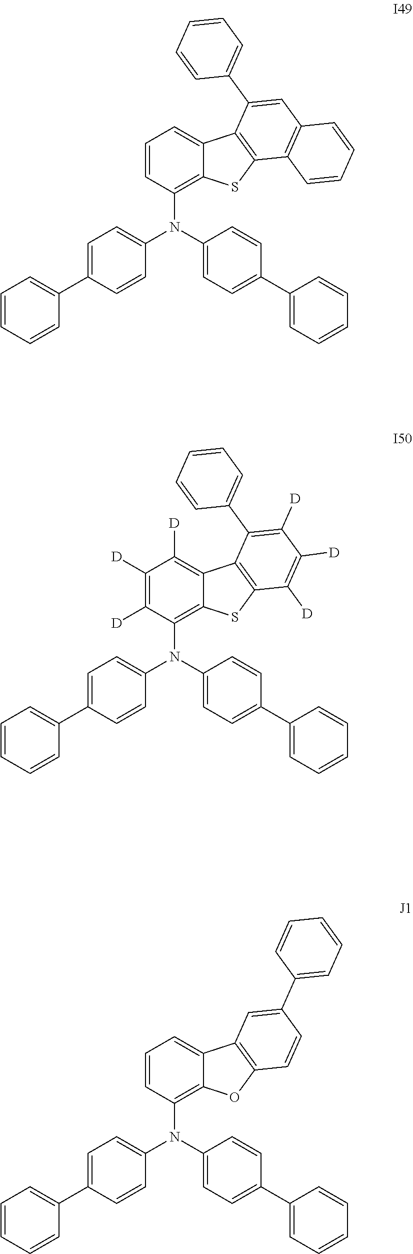 [SMILES: c1ccc(-c2ccc(N(c3ccc(-c4ccccc4)cc3)c3cccc4c3sc3c5ccccc5cc(-c5ccccc5)c43)cc2)cc1 ; [2H]c1c([2H])c(-c2ccccc2)c2c(sc3c(N(c4ccc(-c5ccccc5)cc4)c4ccc(-c5ccccc5)cc4)c([2H])c([2H])c([2H])c32)c1[2H] ; c1ccc(-c2ccc(N(c3ccc(-c4ccccc4)cc3)c3cccc4c3oc3ccc(-c5ccccc5)cc34)cc2)cc1]

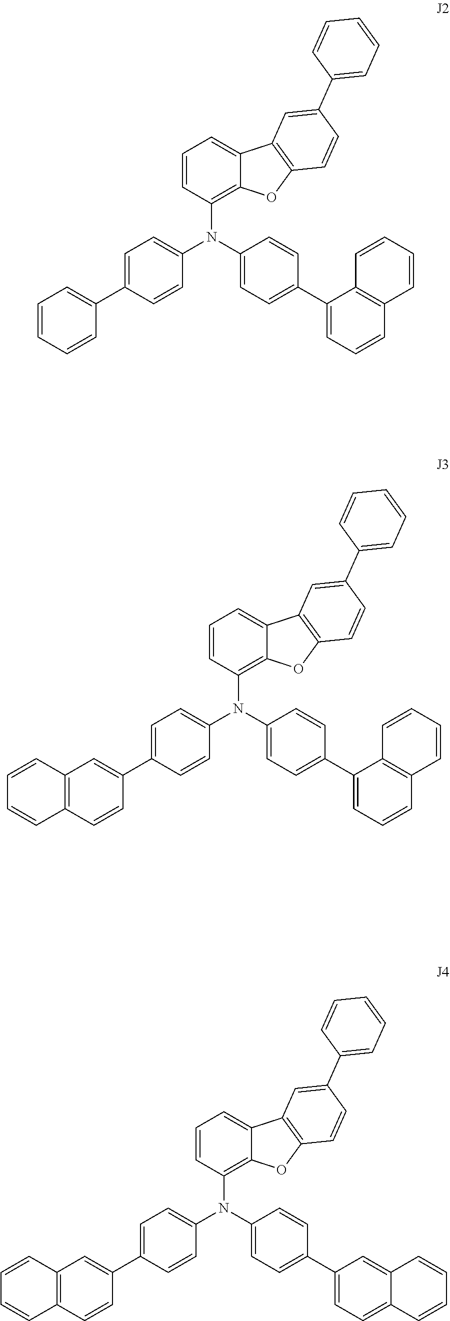 [SMILES: c1ccc(-c2ccc(N(c3ccc(-c4cccc5ccccc45)cc3)c3cccc4c3oc3ccc(-c5ccccc5)cc34)cc2)cc1 ; c1ccc(-c2ccc3oc4c(N(c5ccc(-c6ccc7ccccc7c6)cc5)c5ccc(-c6cccc7ccccc67)cc5)cccc4c3c2)cc1 ; c1ccc(-c2ccc3oc4c(N(c5ccc(-c6ccc7ccccc7c6)cc5)c5ccc(-c6ccc7ccccc7c6)cc5)cccc4c3c2)cc1]

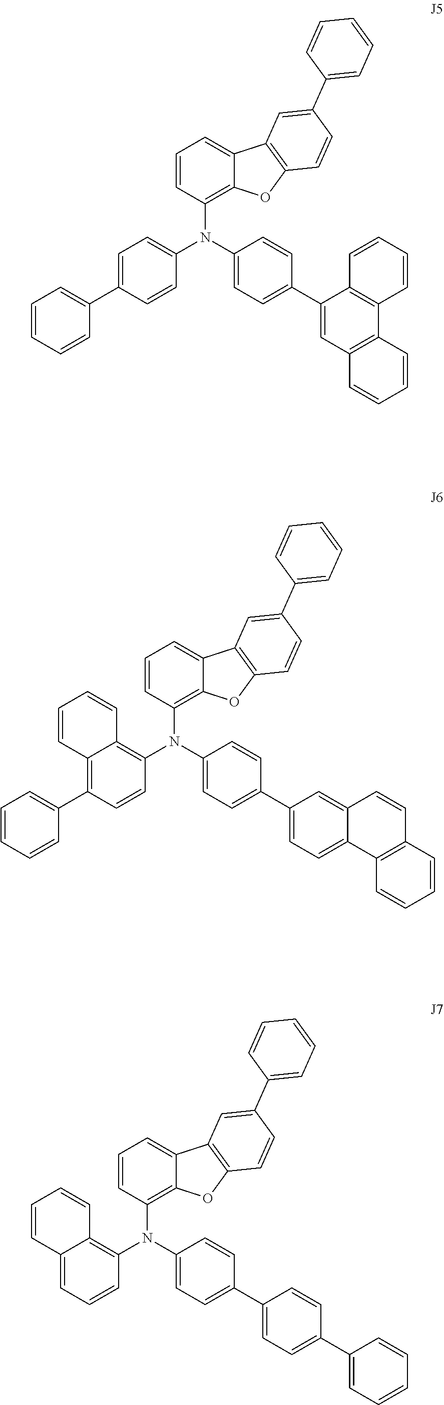 [SMILES: c1ccc(-c2ccc(N(c3ccc(-c4cc5ccccc5c5ccccc45)cc3)c3cccc4c3oc3ccc(-c5ccccc5)cc34)cc2)cc1 ; c1ccc(-c2ccc3oc4c(N(c5ccc(-c6ccc7c(ccc8ccccc87)c6)cc5)c5ccc(-c6ccccc6)c6ccccc56)cccc4c3c2)cc1 ; c1ccc(-c2ccc(-c3ccc(N(c4cccc5ccccc45)c4cccc5c4oc4ccc(-c6ccccc6)cc45)cc3)cc2)cc1]

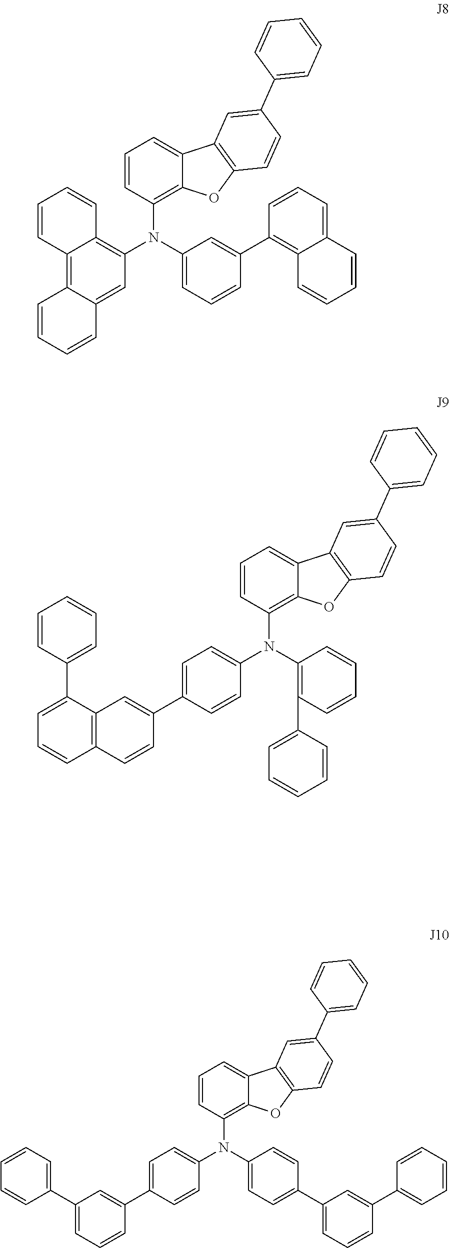 [SMILES: c1ccc(-c2ccc3oc4c(N(c5cccc(-c6cccc7ccccc67)c5)c5cc6ccccc6c6ccccc56)cccc4c3c2)cc1 ; c1ccc(-c2ccc3oc4c(N(c5ccc(-c6ccc7cccc(-c8ccccc8)c7c6)cc5)c5ccccc5-c5ccccc5)cccc4c3c2)cc1 ; c1ccc(-c2cccc(-c3ccc(N(c4ccc(-c5cccc(-c6ccccc6)c5)cc4)c4cccc5c4oc4ccc(-c6ccccc6)cc45)cc3)c2)cc1]

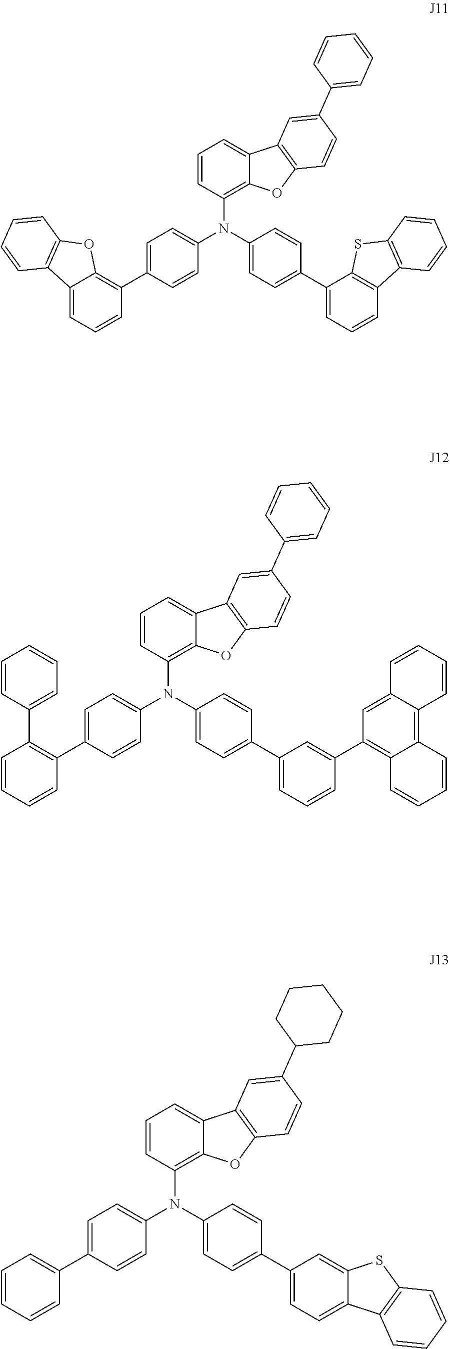 [SMILES: c1ccc(-c2ccc3oc4c(N(c5ccc(-c6cccc7c6oc6ccccc67)cc5)c5ccc(-c6cccc7c6sc6ccccc67)cc5)cccc4c3c2)cc1 ; c1ccc(-c2ccc3oc4c(N(c5ccc(-c6cccc(-c7cc8ccccc8c8ccccc78)c6)cc5)c5ccc(-c6ccccc6-c6ccccc6)cc5)cccc4c3c2)cc1 ; c1ccc(-c2ccc(N(c3ccc(-c4ccc5c(c4)sc4ccccc45)cc3)c3cccc4c3oc3ccc(C5CCCCC5)cc34)cc2)cc1]

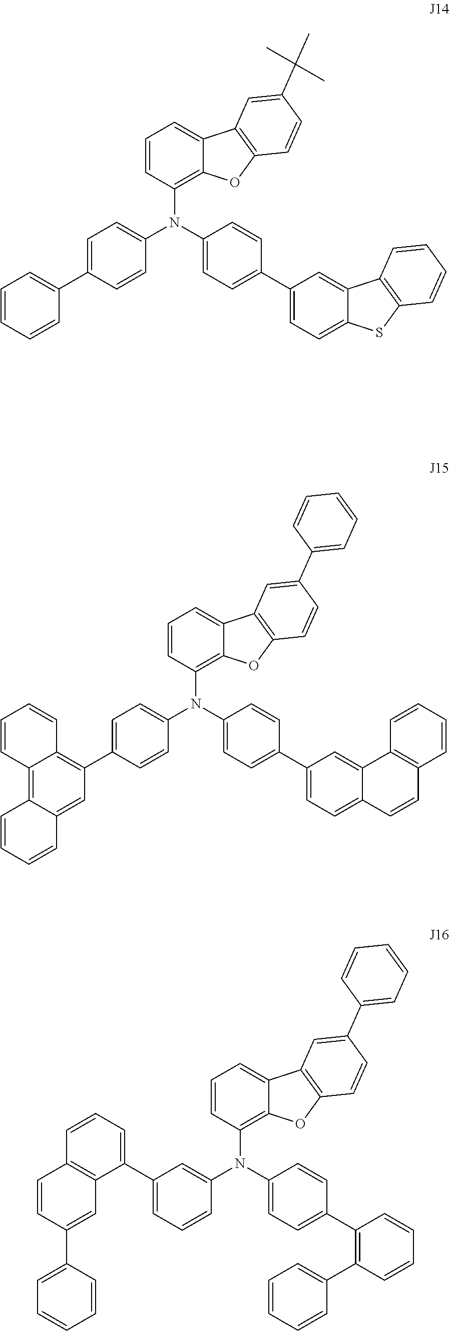 [SMILES: CC(C)(C)c1ccc2oc3c(N(c4ccc(-c5ccccc5)cc4)c4ccc(-c5ccc6sc7ccccc7c6c5)cc4)cccc3c2c1 ; c1ccc(-c2ccc3oc4c(N(c5ccc(-c6ccc7ccc8ccccc8c7c6)cc5)c5ccc(-c6cc7ccccc7c7ccccc67)cc5)cccc4c3c2)cc1 ; c1ccc(-c2ccc3cccc(-c4cccc(N(c5ccc(-c6ccccc6-c6ccccc6)cc5)c5cccc6c5oc5ccc(-c7ccccc7)cc56)c4)c3c2)cc1]

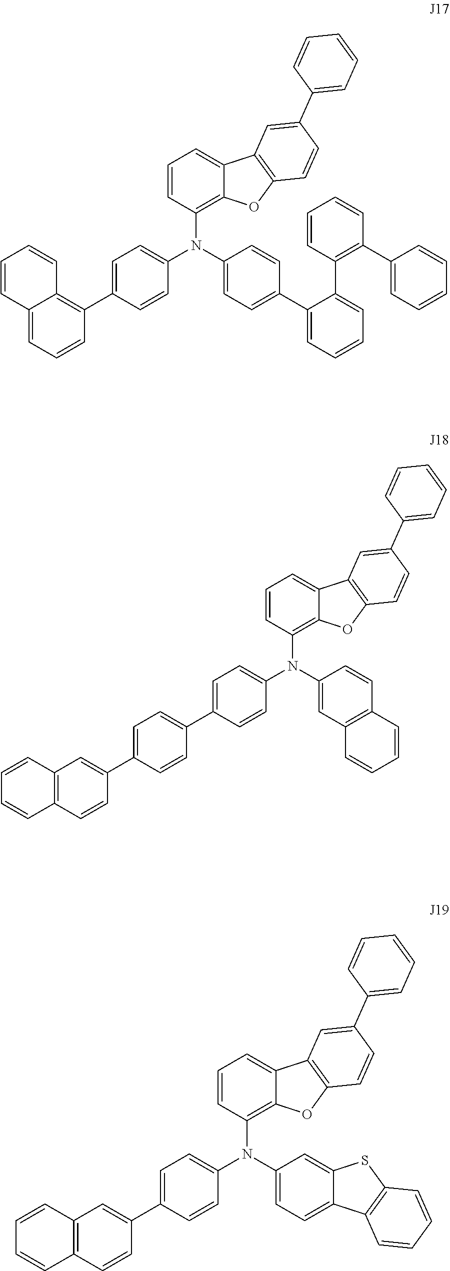 [SMILES: c1ccc(-c2ccc3oc4c(N(c5ccc(-c6ccccc6-c6ccccc6-c6ccccc6)cc5)c5ccc(-c6cccc7ccccc67)cc5)cccc4c3c2)cc1 ; c1ccc(-c2ccc3oc4c(N(c5ccc(-c6ccc(-c7ccc8ccccc8c7)cc6)cc5)c5ccc6ccccc6c5)cccc4c3c2)cc1 ; c1ccc(-c2ccc3oc4c(N(c5ccc(-c6ccc7ccccc7c6)cc5)c5ccc6c(c5)sc5ccccc56)cccc4c3c2)cc1]

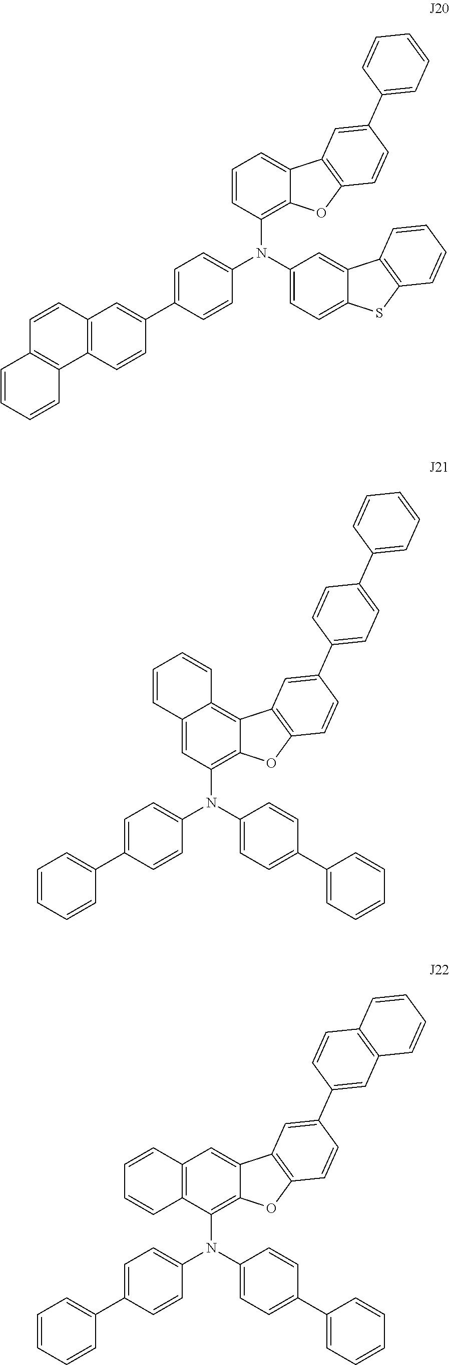 [SMILES: c1ccc(-c2ccc3oc4c(N(c5ccc(-c6ccc7c(ccc8ccccc87)c6)cc5)c5ccc6sc7ccccc7c6c5)cccc4c3c2)cc1 ; c1ccc(-c2ccc(-c3ccc4oc5c(N(c6ccc(-c7ccccc7)cc6)c6ccc(-c7ccccc7)cc6)cc6ccccc6c5c4c3)cc2)cc1 ; c1ccc(-c2ccc(N(c3ccc(-c4ccccc4)cc3)c3c4ccccc4cc4c3oc3ccc(-c5ccc6ccccc6c5)cc34)cc2)cc1]

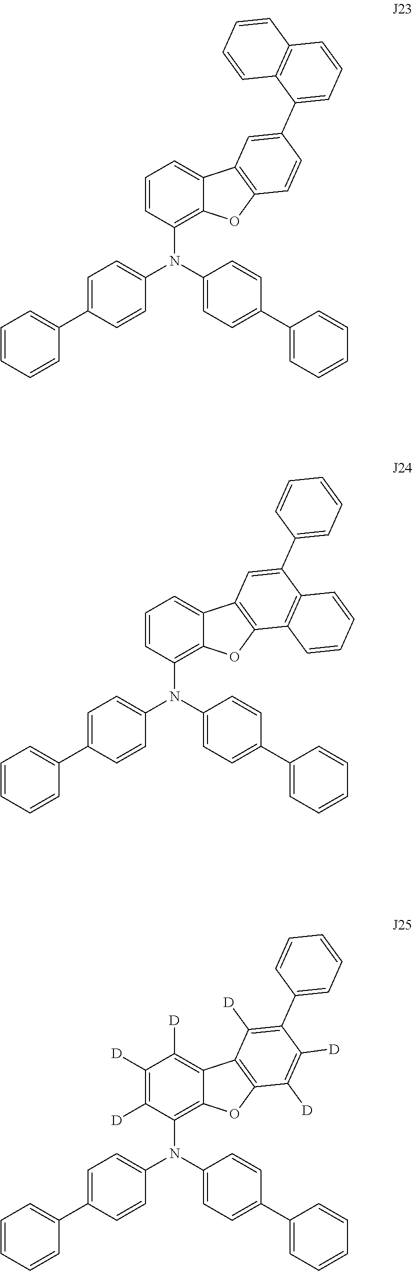 [SMILES: c1ccc(-c2ccc(N(c3ccc(-c4ccccc4)cc3)c3cccc4c3oc3ccc(-c5cccc6ccccc56)cc34)cc2)cc1 ; c1ccc(-c2ccc(N(c3ccc(-c4ccccc4)cc3)c3cccc4c3oc3c5ccccc5c(-c5ccccc5)cc43)cc2)cc1 ; [2H]c1c(-c2ccccc2)c([2H])c2c(oc3c(N(c4ccc(-c5ccccc5)cc4)c4ccc(-c5ccccc5)cc4)c([2H])c([2H])c([2H])c32)c1[2H]]

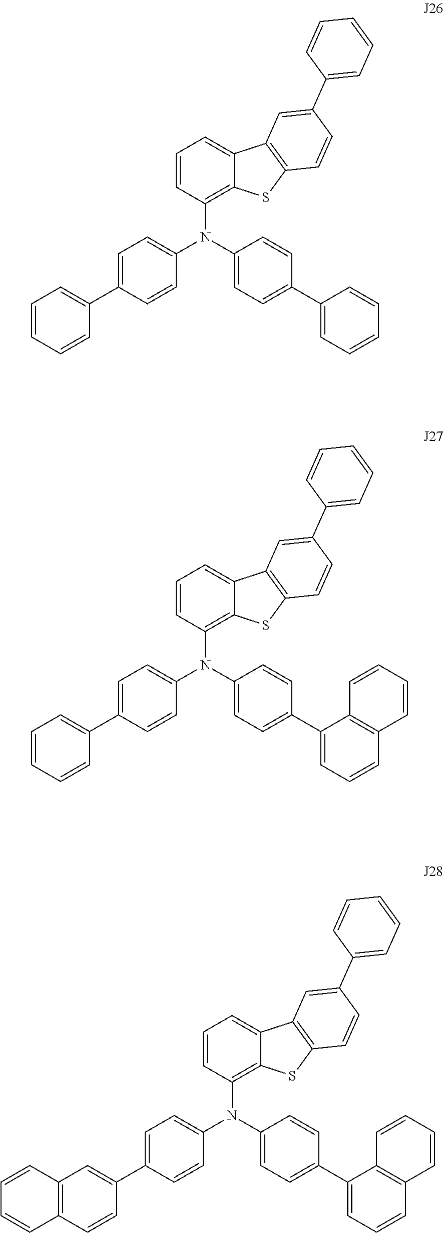 [SMILES: c1ccc(-c2ccc(N(c3ccc(-c4ccccc4)cc3)c3cccc4c3sc3ccc(-c5ccccc5)cc34)cc2)cc1 ; c1ccc(-c2ccc(N(c3ccc(-c4cccc5ccccc45)cc3)c3cccc4c3sc3ccc(-c5ccccc5)cc34)cc2)cc1 ; c1ccc(-c2ccc3sc4c(N(c5ccc(-c6ccc7ccccc7c6)cc5)c5ccc(-c6cccc7ccccc67)cc5)cccc4c3c2)cc1]

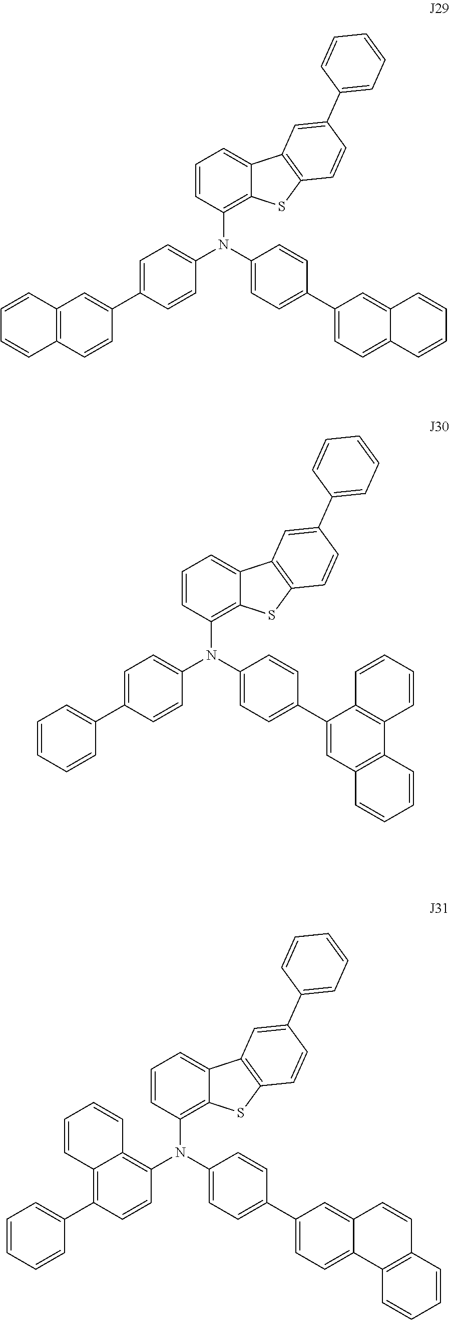 [SMILES: c1ccc(-c2ccc3sc4c(N(c5ccc(-c6ccc7ccccc7c6)cc5)c5ccc(-c6ccc7ccccc7c6)cc5)cccc4c3c2)cc1 ; c1ccc(-c2ccc(N(c3ccc(-c4cc5ccccc5c5ccccc45)cc3)c3cccc4c3sc3ccc(-c5ccccc5)cc34)cc2)cc1 ; c1ccc(-c2ccc3sc4c(N(c5ccc(-c6ccc7c(ccc8ccccc87)c6)cc5)c5ccc(-c6ccccc6)c6ccccc56)cccc4c3c2)cc1]

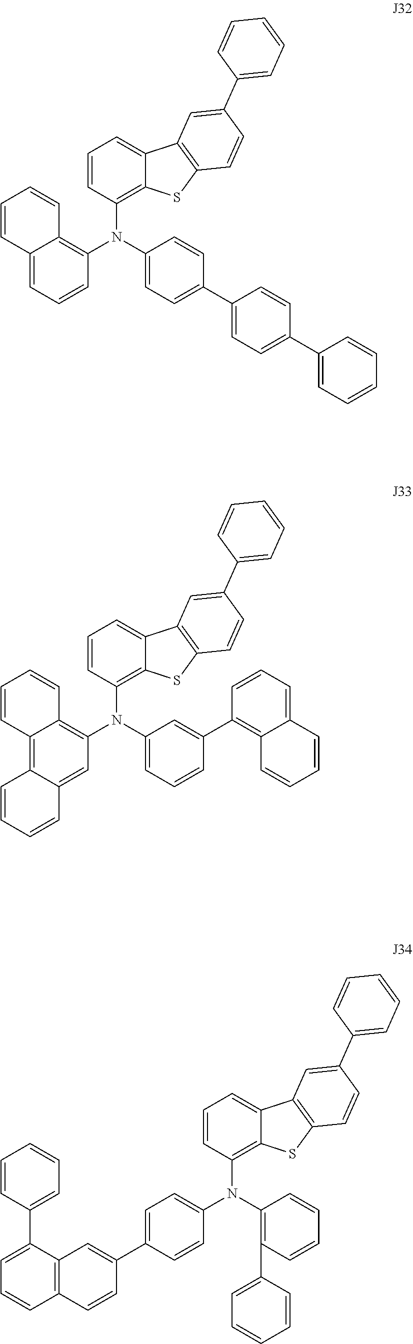 [SMILES: c1ccc(-c2ccc(-c3ccc(N(c4cccc5ccccc45)c4cccc5c4sc4ccc(-c6ccccc6)cc45)cc3)cc2)cc1 ; c1ccc(-c2ccc3sc4c(N(c5cccc(-c6cccc7ccccc67)c5)c5cc6ccccc6c6ccccc56)cccc4c3c2)cc1 ; c1ccc(-c2ccc3sc4c(N(c5ccc(-c6ccc7cccc(-c8ccccc8)c7c6)cc5)c5ccccc5-c5ccccc5)cccc4c3c2)cc1]

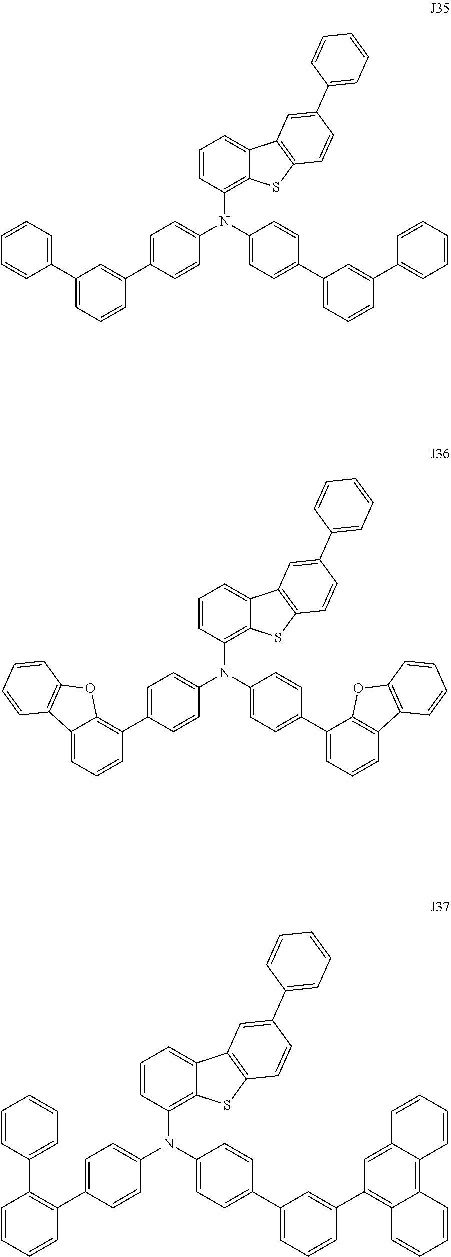 [SMILES: c1ccc(-c2cccc(-c3ccc(N(c4ccc(-c5cccc(-c6ccccc6)c5)cc4)c4cccc5c4sc4ccc(-c6ccccc6)cc45)cc3)c2)cc1 ; c1ccc(-c2ccc3sc4c(N(c5ccc(-c6cccc7c6oc6ccccc67)cc5)c5ccc(-c6cccc7c6oc6ccccc67)cc5)cccc4c3c2)cc1 ; c1ccc(-c2ccc3sc4c(N(c5ccc(-c6cccc(-c7cc8ccccc8c8ccccc78)c6)cc5)c5ccc(-c6ccccc6-c6ccccc6)cc5)cccc4c3c2)cc1]

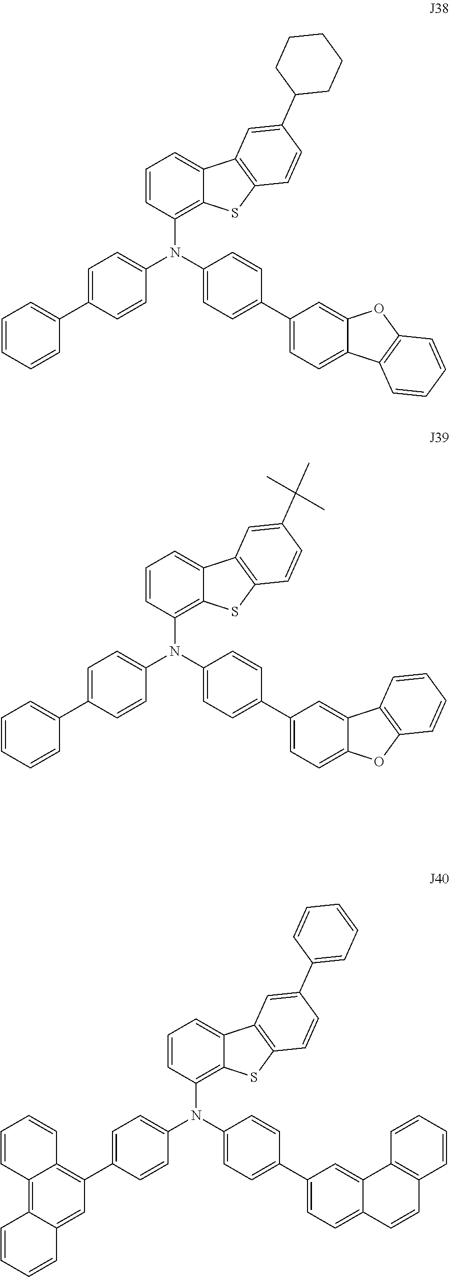 [SMILES: c1ccc(-c2ccc(N(c3ccc(-c4ccc5c(c4)oc4ccccc45)cc3)c3cccc4c3sc3ccc(C5CCCCC5)cc34)cc2)cc1 ; CC(C)(C)c1ccc2sc3c(N(c4ccc(-c5ccccc5)cc4)c4ccc(-c5ccc6oc7ccccc7c6c5)cc4)cccc3c2c1 ; c1ccc(-c2ccc3sc4c(N(c5ccc(-c6ccc7ccc8ccccc8c7c6)cc5)c5ccc(-c6cc7ccccc7c7ccccc67)cc5)cccc4c3c2)cc1]

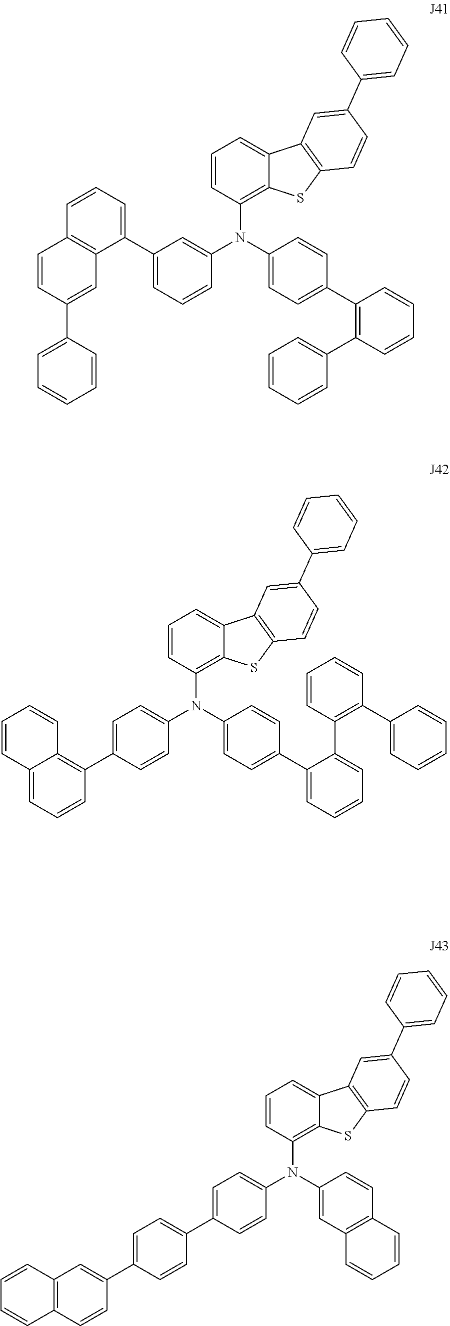 [SMILES: c1ccc(-c2ccc3cccc(-c4cccc(N(c5ccc(-c6ccccc6-c6ccccc6)cc5)c5cccc6c5sc5ccc(-c7ccccc7)cc56)c4)c3c2)cc1 ; c1ccc(-c2ccc3sc4c(N(c5ccc(-c6ccccc6-c6ccccc6-c6ccccc6)cc5)c5ccc(-c6cccc7ccccc67)cc5)cccc4c3c2)cc1 ; c1ccc(-c2ccc3sc4c(N(c5ccc(-c6ccc(-c7ccc8ccccc8c7)cc6)cc5)c5ccc6ccccc6c5)cccc4c3c2)cc1]

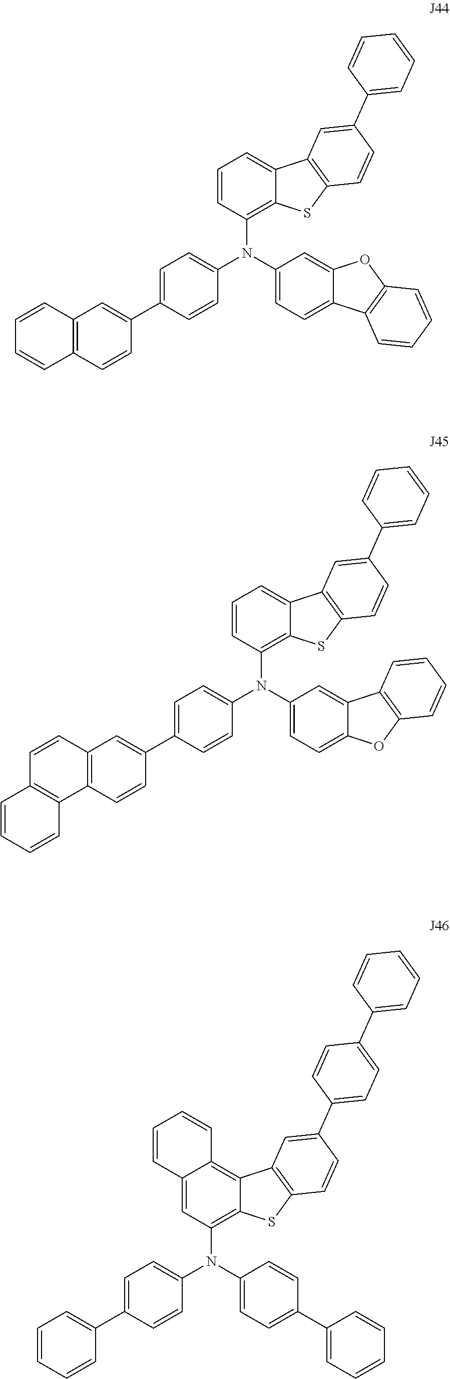 [SMILES: c1ccc(-c2ccc3sc4c(N(c5ccc(-c6ccc7ccccc7c6)cc5)c5ccc6c(c5)oc5ccccc56)cccc4c3c2)cc1 ; c1ccc(-c2ccc3sc4c(N(c5ccc(-c6ccc7c(ccc8ccccc87)c6)cc5)c5ccc6oc7ccccc7c6c5)cccc4c3c2)cc1 ; c1ccc(-c2ccc(-c3ccc4sc5c(N(c6ccc(-c7ccccc7)cc6)c6ccc(-c7ccccc7)cc6)cc6ccccc6c5c4c3)cc2)cc1]

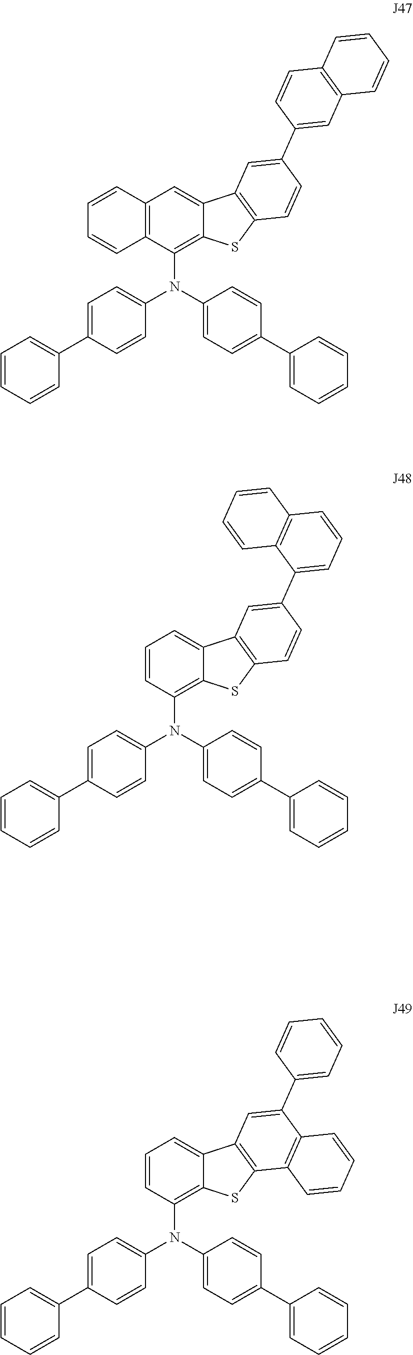 [SMILES: c1ccc(-c2ccc(N(c3ccc(-c4ccccc4)cc3)c3c4ccccc4cc4c3sc3ccc(-c5ccc6ccccc6c5)cc34)cc2)cc1 ; c1ccc(-c2ccc(N(c3ccc(-c4ccccc4)cc3)c3cccc4c3sc3ccc(-c5cccc6ccccc56)cc34)cc2)cc1 ; c1ccc(-c2ccc(N(c3ccc(-c4ccccc4)cc3)c3cccc4c3sc3c5ccccc5c(-c5ccccc5)cc43)cc2)cc1]

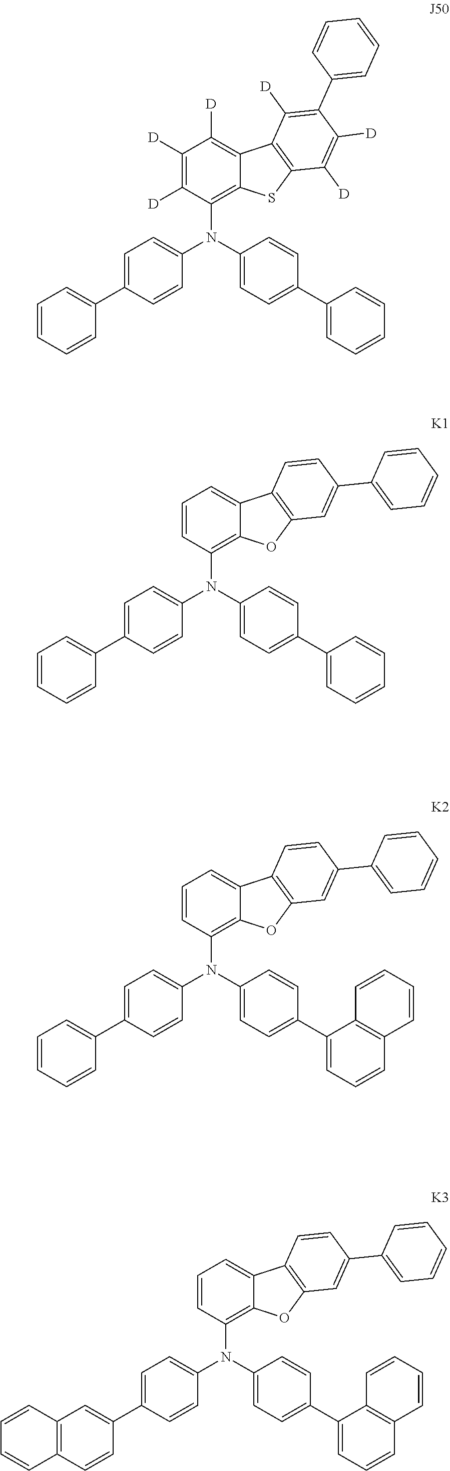 [SMILES: [2H]c1c(-c2ccccc2)c([2H])c2c(sc3c(N(c4ccc(-c5ccccc5)cc4)c4ccc(-c5ccccc5)cc4)c([2H])c([2H])c([2H])c32)c1[2H] ; c1ccc(-c2ccc(N(c3ccc(-c4ccccc4)cc3)c3cccc4c3oc3cc(-c5ccccc5)ccc34)cc2)cc1 ; c1ccc(-c2ccc(N(c3ccc(-c4cccc5ccccc45)cc3)c3cccc4c3oc3cc(-c5ccccc5)ccc34)cc2)cc1 ; c1ccc(-c2ccc3c(c2)oc2c(N(c4ccc(-c5ccc6ccccc6c5)cc4)c4ccc(-c5cccc6ccccc56)cc4)cccc23)cc1]

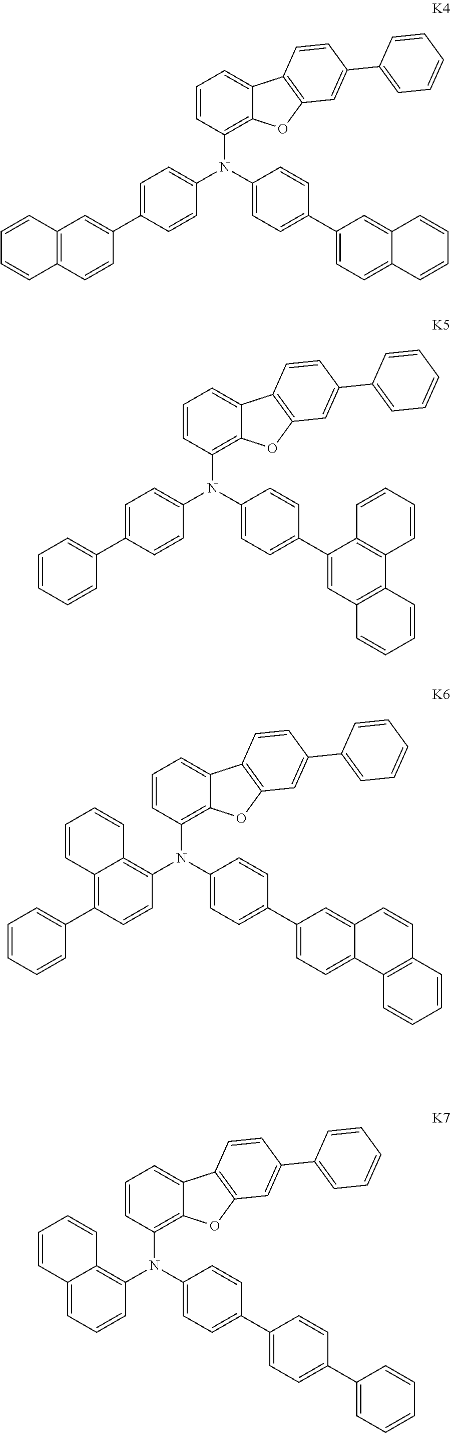 [SMILES: c1ccc(-c2ccc3c(c2)oc2c(N(c4ccc(-c5ccc6ccccc6c5)cc4)c4ccc(-c5ccc6ccccc6c5)cc4)cccc23)cc1 ; c1ccc(-c2ccc(N(c3ccc(-c4cc5ccccc5c5ccccc45)cc3)c3cccc4c3oc3cc(-c5ccccc5)ccc34)cc2)cc1 ; c1ccc(-c2ccc3c(c2)oc2c(N(c4ccc(-c5ccc6c(ccc7ccccc76)c5)cc4)c4ccc(-c5ccccc5)c5ccccc45)cccc23)cc1 ; c1ccc(-c2ccc(-c3ccc(N(c4cccc5ccccc45)c4cccc5c4oc4cc(-c6ccccc6)ccc45)cc3)cc2)cc1]

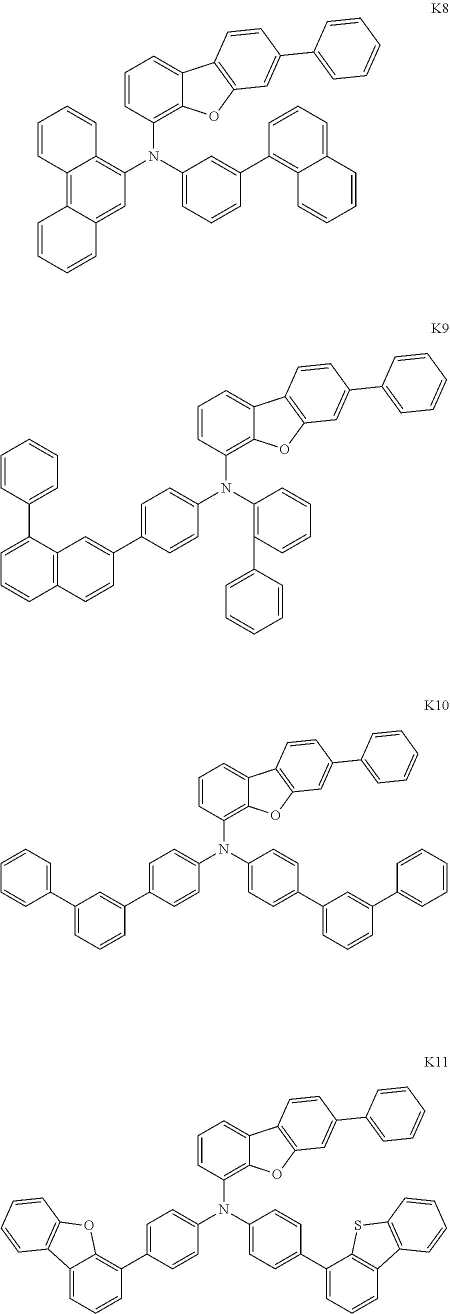 [SMILES: c1ccc(-c2ccc3c(c2)oc2c(N(c4cccc(-c5cccc6ccccc56)c4)c4cc5ccccc5c5ccccc45)cccc23)cc1 ; c1ccc(-c2ccc3c(c2)oc2c(N(c4ccc(-c5ccc6cccc(-c7ccccc7)c6c5)cc4)c4ccccc4-c4ccccc4)cccc23)cc1 ; c1ccc(-c2cccc(-c3ccc(N(c4ccc(-c5cccc(-c6ccccc6)c5)cc4)c4cccc5c4oc4cc(-c6ccccc6)ccc45)cc3)c2)cc1 ; c1ccc(-c2ccc3c(c2)oc2c(N(c4ccc(-c5cccc6c5oc5ccccc56)cc4)c4ccc(-c5cccc6c5sc5ccccc56)cc4)cccc23)cc1]

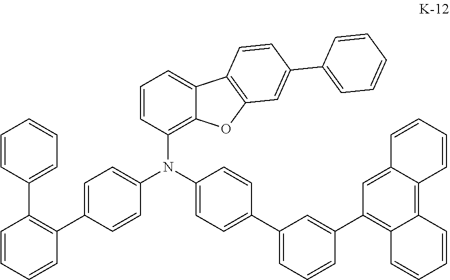 [SMILES: c1ccc(-c2ccc3c(c2)oc2c(N(c4ccc(-c5cccc(-c6cc7ccccc7c7ccccc67)c5)cc4)c4ccc(-c5ccccc5-c5ccccc5)cc4)cccc23)cc1]

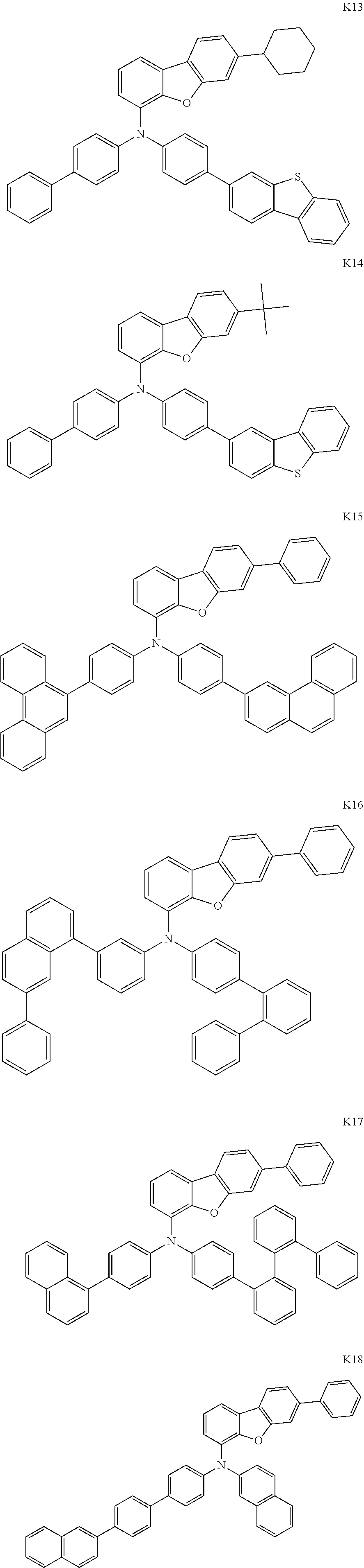

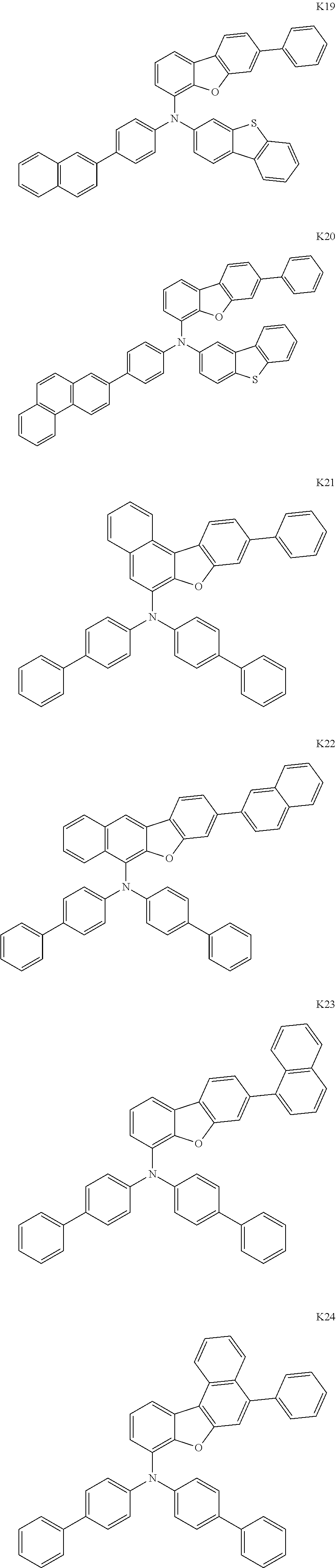

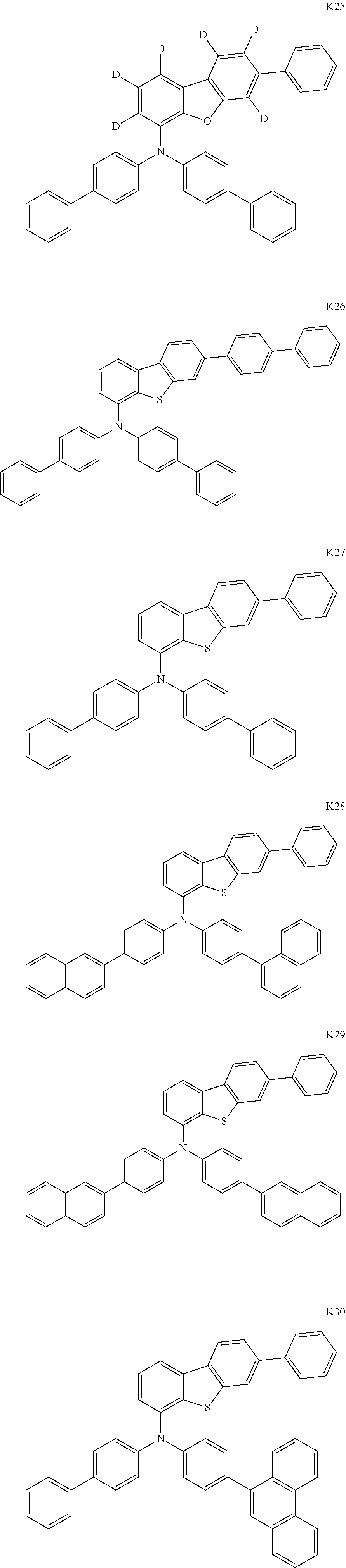

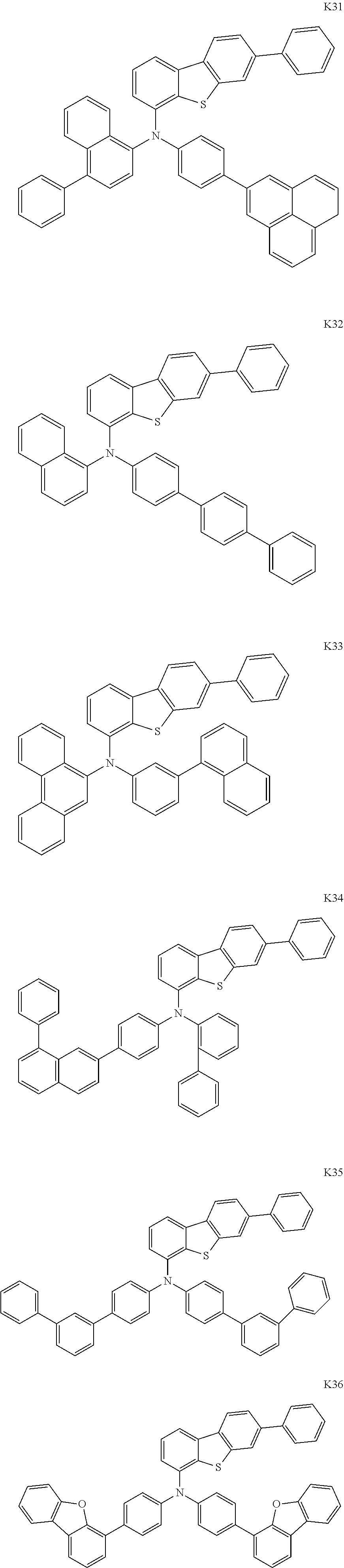

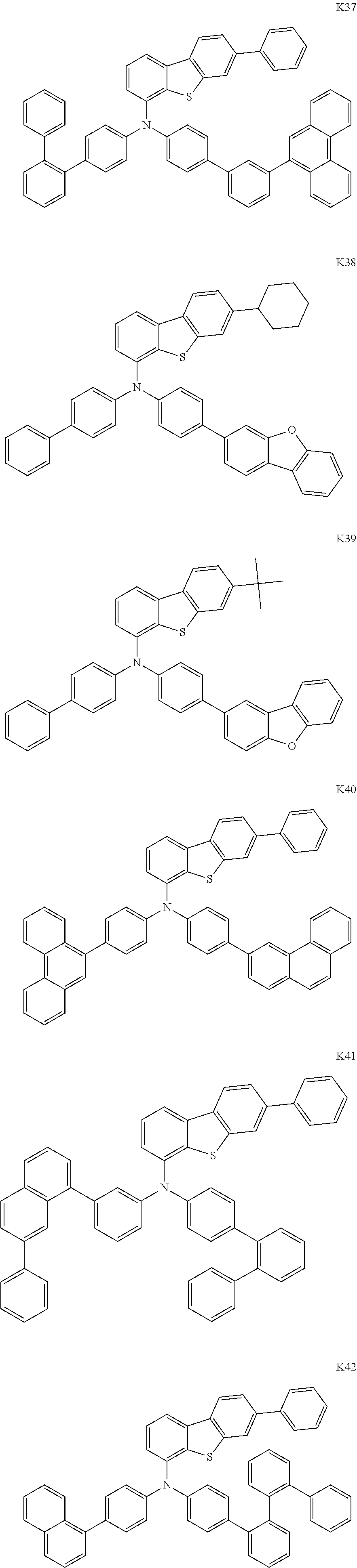

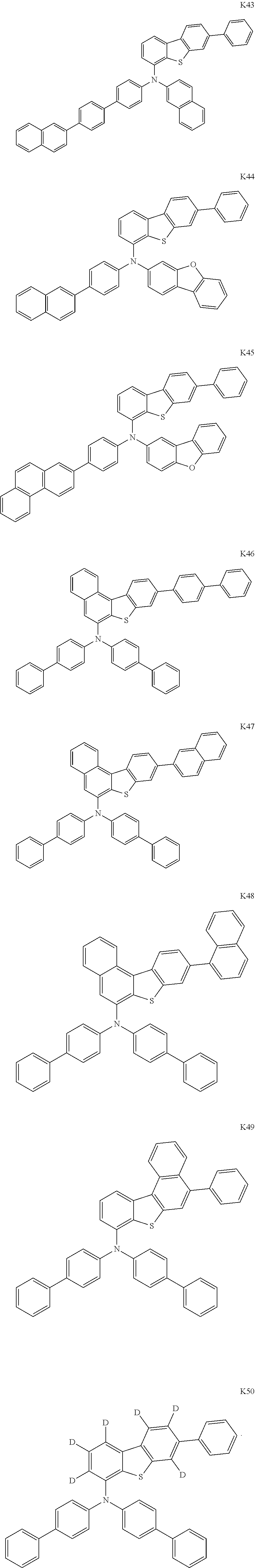

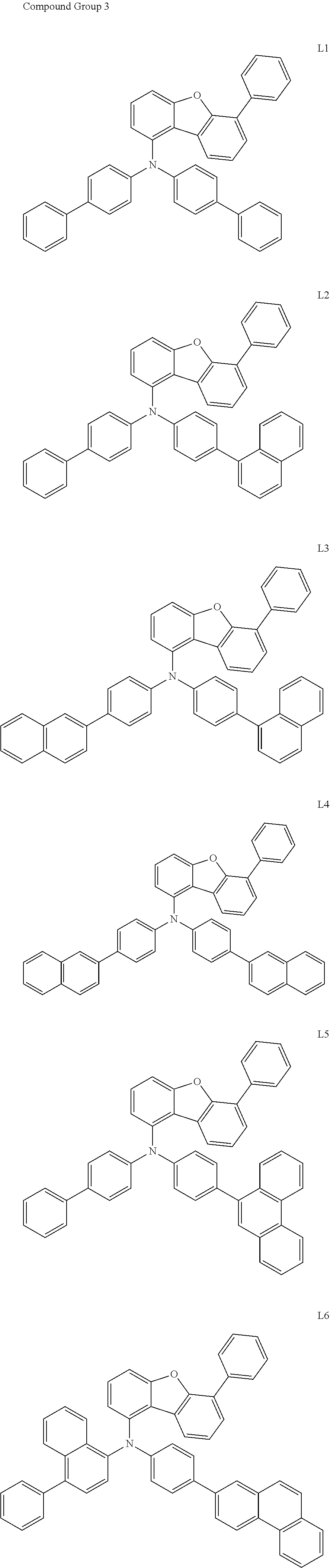

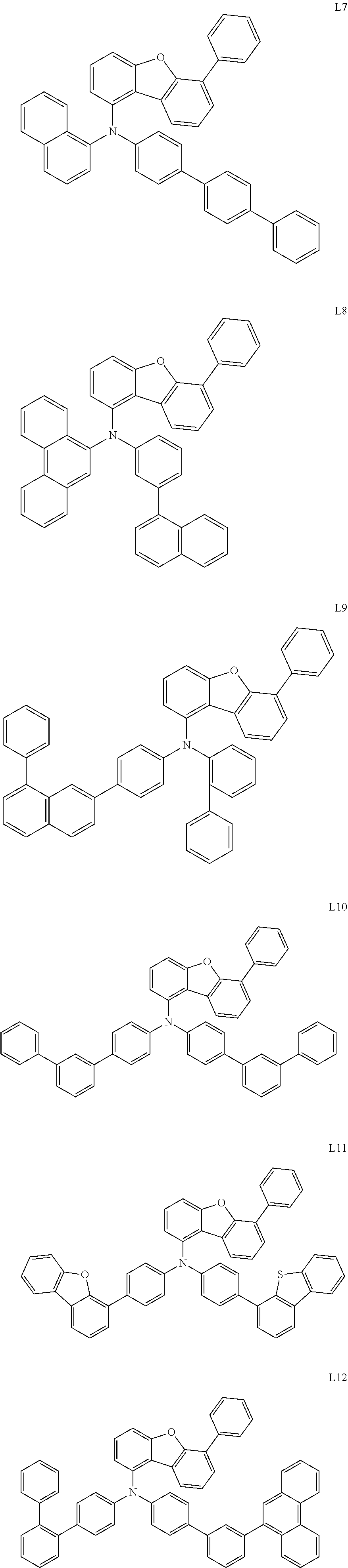

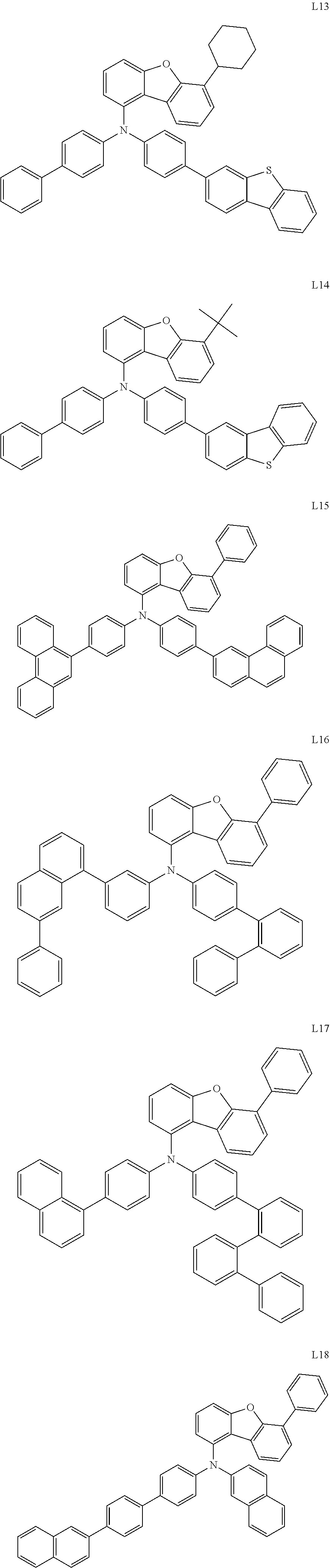

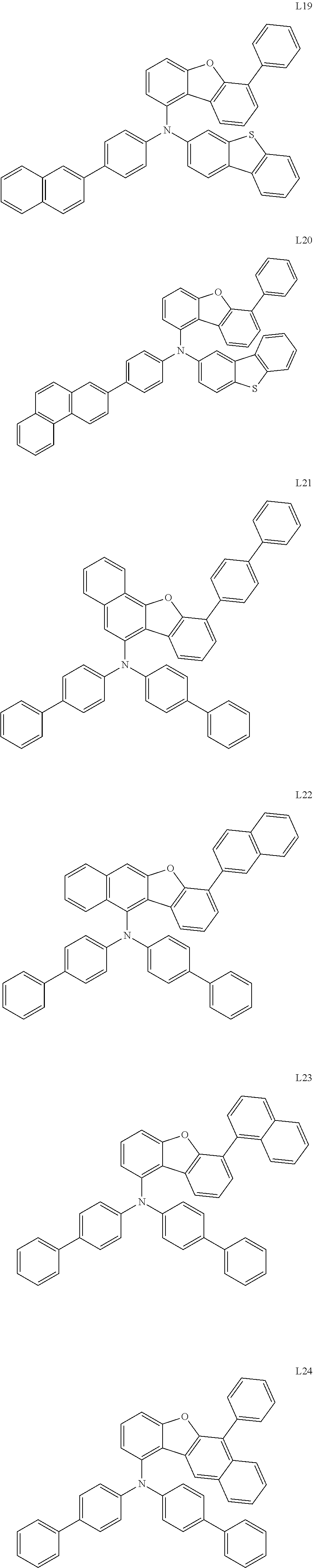

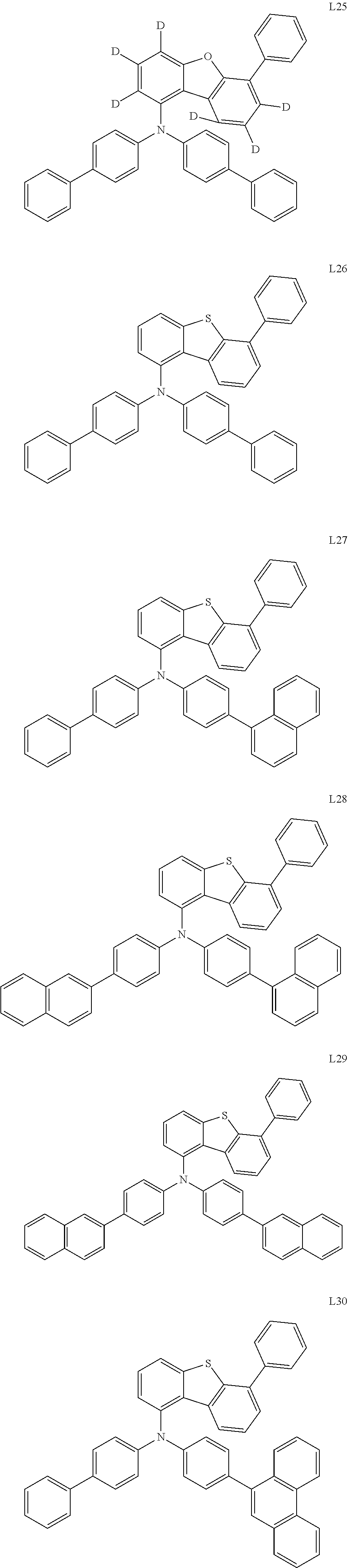

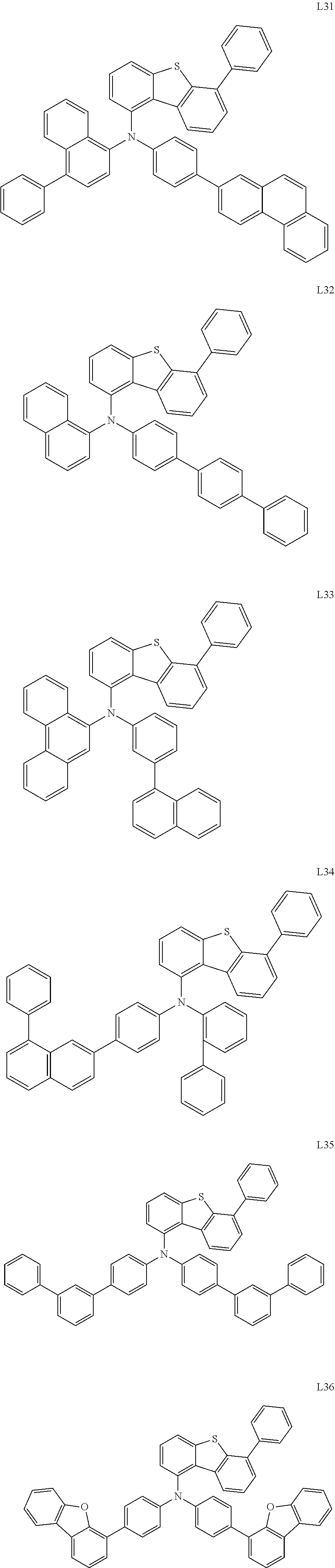

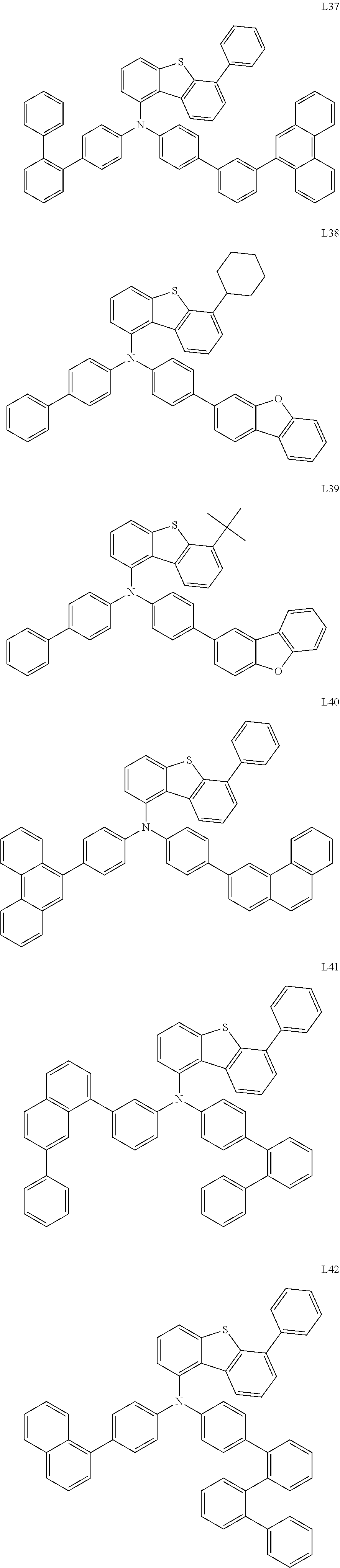

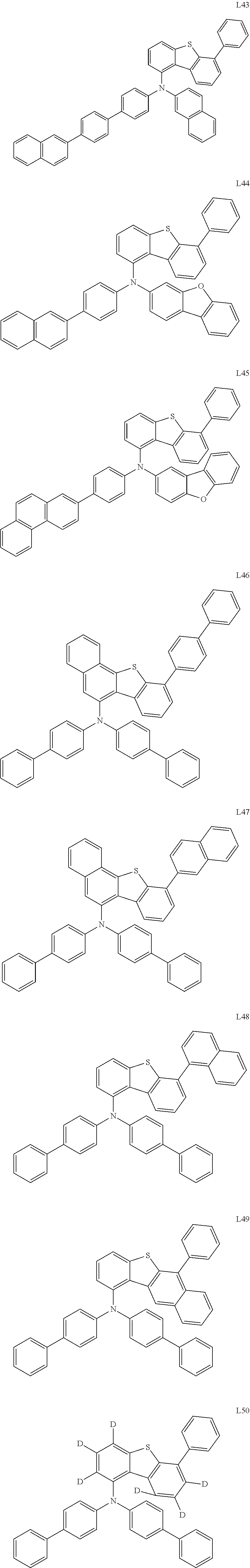

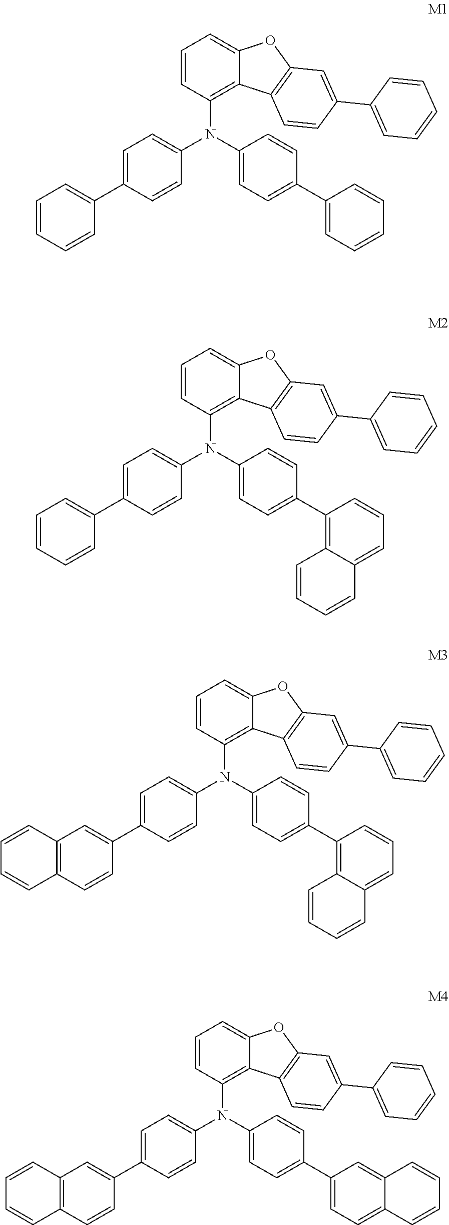 [SMILES: c1ccc(-c2ccc(N(c3ccc(-c4ccccc4)cc3)c3cccc4oc5cc(-c6ccccc6)ccc5c34)cc2)cc1 ; c1ccc(-c2ccc(N(c3ccc(-c4cccc5ccccc45)cc3)c3cccc4oc5cc(-c6ccccc6)ccc5c34)cc2)cc1 ; c1ccc(-c2ccc3c(c2)oc2cccc(N(c4ccc(-c5ccc6ccccc6c5)cc4)c4ccc(-c5cccc6ccccc56)cc4)c23)cc1 ; c1ccc(-c2ccc3c(c2)oc2cccc(N(c4ccc(-c5ccc6ccccc6c5)cc4)c4ccc(-c5ccc6ccccc6c5)cc4)c23)cc1]

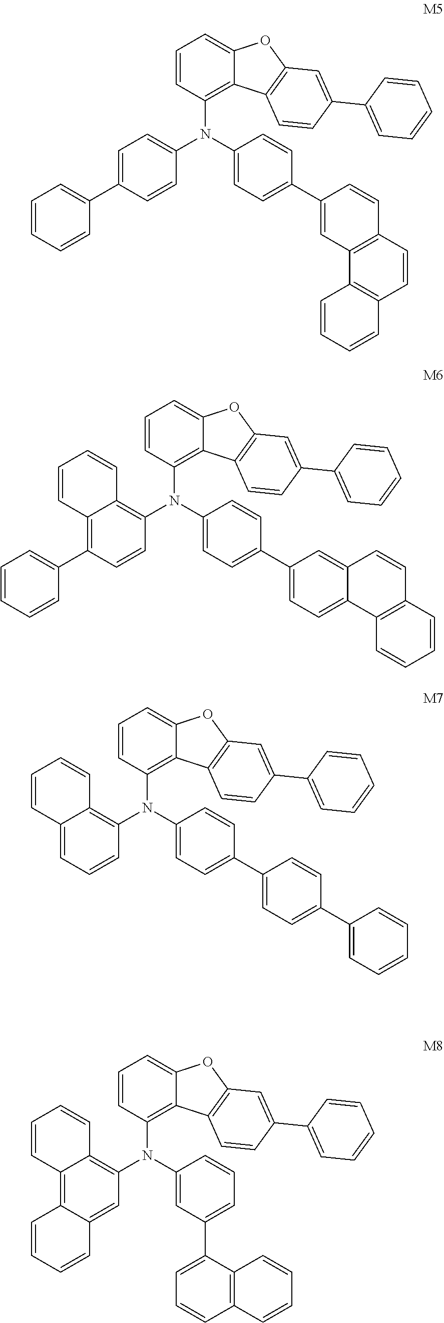 [SMILES: c1ccc(-c2ccc(N(c3ccc(-c4ccc5ccc6ccccc6c5c4)cc3)c3cccc4oc5cc(-c6ccccc6)ccc5c34)cc2)cc1 ; c1ccc(-c2ccc3c(c2)oc2cccc(N(c4ccc(-c5ccc6c(ccc7ccccc76)c5)cc4)c4ccc(-c5ccccc5)c5ccccc45)c23)cc1 ; c1ccc(-c2ccc(-c3ccc(N(c4cccc5ccccc45)c4cccc5oc6cc(-c7ccccc7)ccc6c45)cc3)cc2)cc1 ; c1ccc(-c2ccc3c(c2)oc2cccc(N(c4cccc(-c5cccc6ccccc56)c4)c4cc5ccccc5c5ccccc45)c23)cc1]

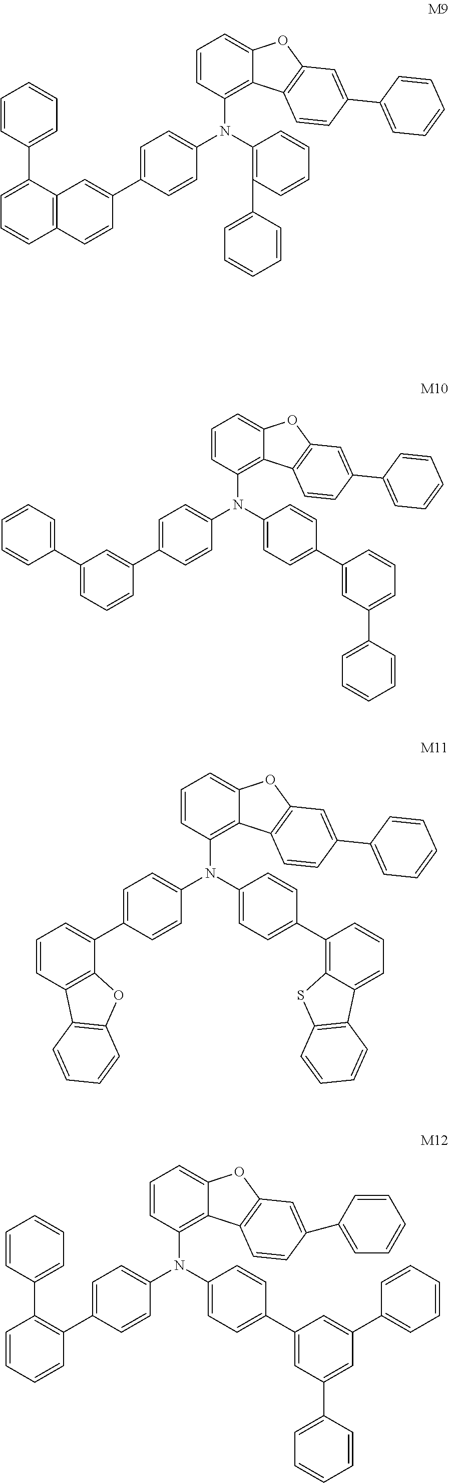 [SMILES: c1ccc(-c2ccc3c(c2)oc2cccc(N(c4ccc(-c5ccc6cccc(-c7ccccc7)c6c5)cc4)c4ccccc4-c4ccccc4)c23)cc1 ; c1ccc(-c2cccc(-c3ccc(N(c4ccc(-c5cccc(-c6ccccc6)c5)cc4)c4cccc5oc6cc(-c7ccccc7)ccc6c45)cc3)c2)cc1 ; c1ccc(-c2ccc3c(c2)oc2cccc(N(c4ccc(-c5cccc6c5oc5ccccc56)cc4)c4ccc(-c5cccc6c5sc5ccccc56)cc4)c23)cc1 ; c1ccc(-c2cc(-c3ccccc3)cc(-c3ccc(N(c4ccc(-c5ccccc5-c5ccccc5)cc4)c4cccc5oc6cc(-c7ccccc7)ccc6c45)cc3)c2)cc1]

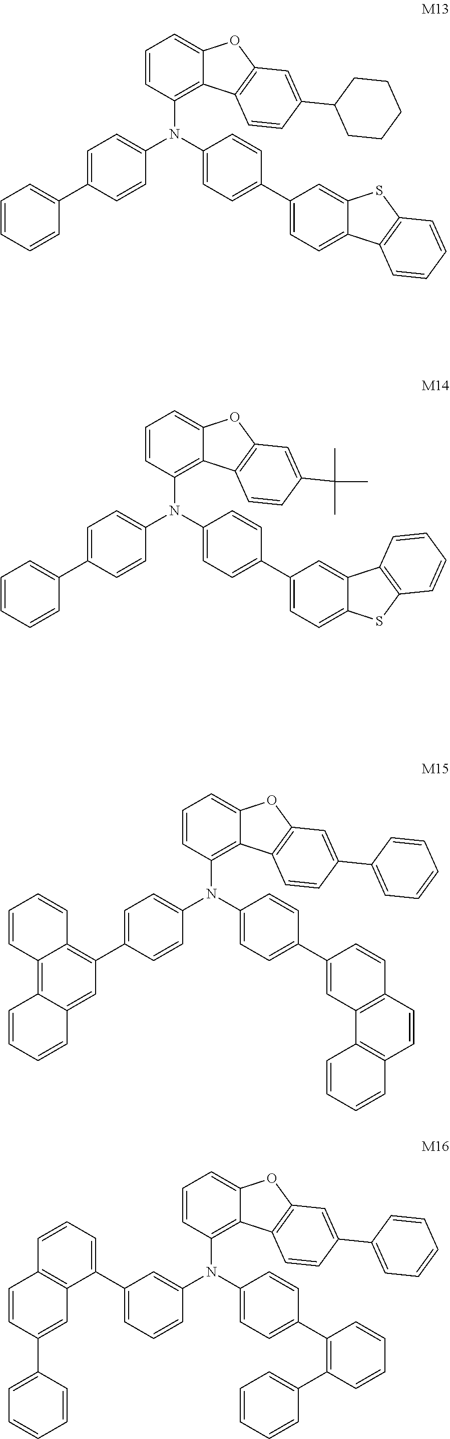 [SMILES: c1ccc(-c2ccc(N(c3ccc(-c4ccc5c(c4)sc4ccccc45)cc3)c3cccc4oc5cc(C6CCCCC6)ccc5c34)cc2)cc1 ; CC(C)(C)c1ccc2c(c1)oc1cccc(N(c3ccc(-c4ccccc4)cc3)c3ccc(-c4ccc5sc6ccccc6c5c4)cc3)c12 ; c1ccc(-c2ccc3c(c2)oc2cccc(N(c4ccc(-c5ccc6ccc7ccccc7c6c5)cc4)c4ccc(-c5cc6ccccc6c6ccccc56)cc4)c23)cc1 ; c1ccc(-c2ccc3c(c2)oc2cccc(N(c4ccc(-c5ccccc5-c5ccccc5)cc4)c4cccc(-c5cccc6ccc(-c7ccccc7)cc56)c4)c23)cc1]

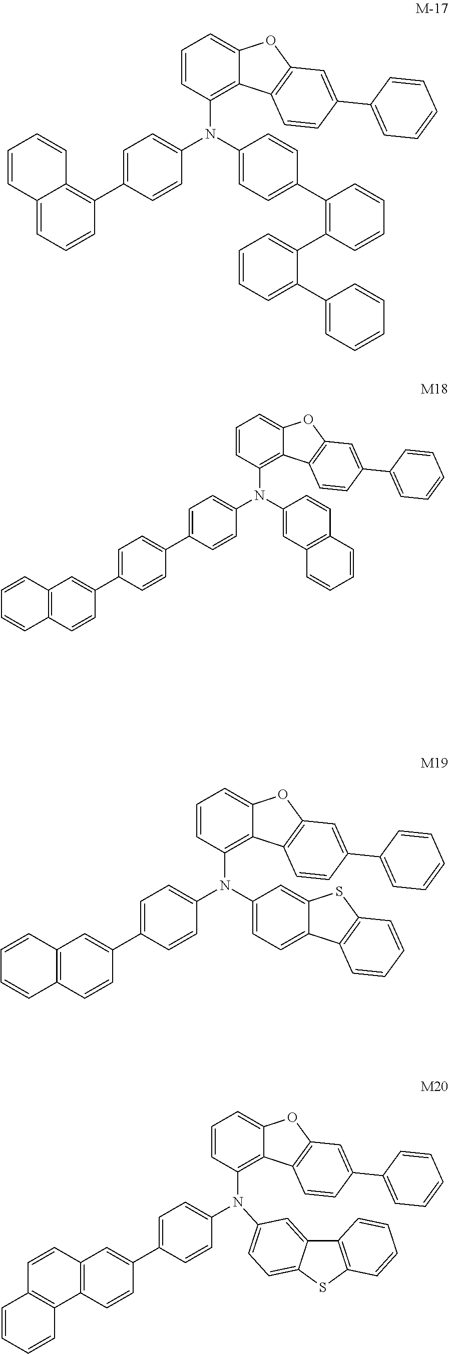 [SMILES: c1ccc(-c2ccc3c(c2)oc2cccc(N(c4ccc(-c5ccccc5-c5ccccc5-c5ccccc5)cc4)c4ccc(-c5cccc6ccccc56)cc4)c23)cc1 ; c1ccc(-c2ccc3c(c2)oc2cccc(N(c4ccc(-c5ccc(-c6ccc7ccccc7c6)cc5)cc4)c4ccc5ccccc5c4)c23)cc1 ; c1ccc(-c2ccc3c(c2)oc2cccc(N(c4ccc(-c5ccc6ccccc6c5)cc4)c4ccc5c(c4)sc4ccccc45)c23)cc1 ; c1ccc(-c2ccc3c(c2)oc2cccc(N(c4ccc(-c5ccc6c(ccc7ccccc76)c5)cc4)c4ccc5sc6ccccc6c5c4)c23)cc1]

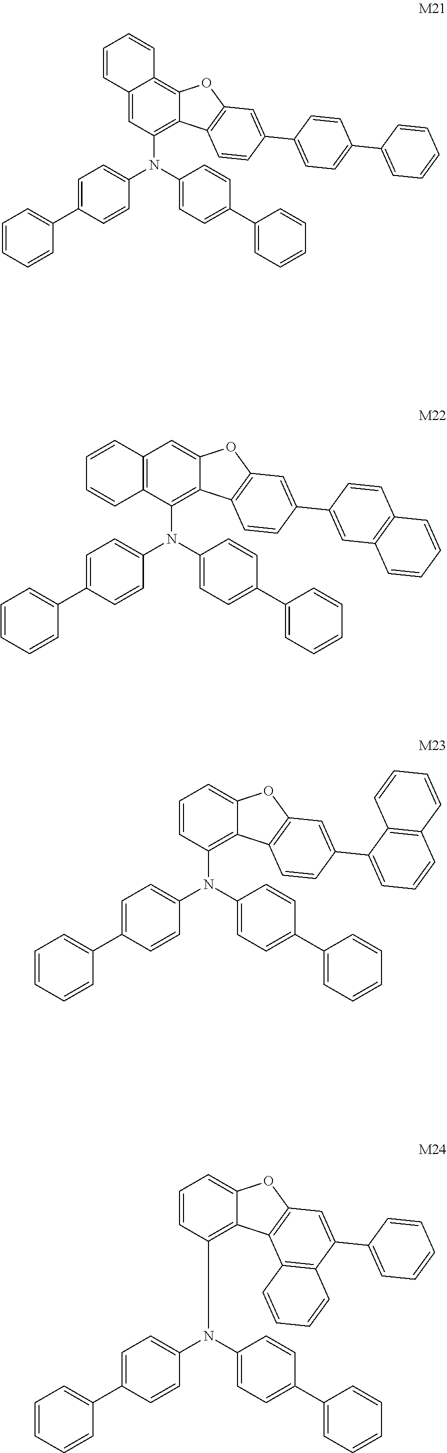 [SMILES: c1ccc(-c2ccc(-c3ccc4c(c3)oc3c5ccccc5cc(N(c5ccc(-c6ccccc6)cc5)c5ccc(-c6ccccc6)cc5)c43)cc2)cc1 ; c1ccc(-c2ccc(N(c3ccc(-c4ccccc4)cc3)c3c4ccccc4cc4oc5cc(-c6ccc7ccccc7c6)ccc5c34)cc2)cc1 ; c1ccc(-c2ccc(N(c3ccc(-c4ccccc4)cc3)c3cccc4oc5cc(-c6cccc7ccccc67)ccc5c34)cc2)cc1 ; c1ccc(-c2ccc(N(c3ccc(-c4ccccc4)cc3)c3cccc4oc5cc(-c6ccccc6)c6ccccc6c5c34)cc2)cc1]

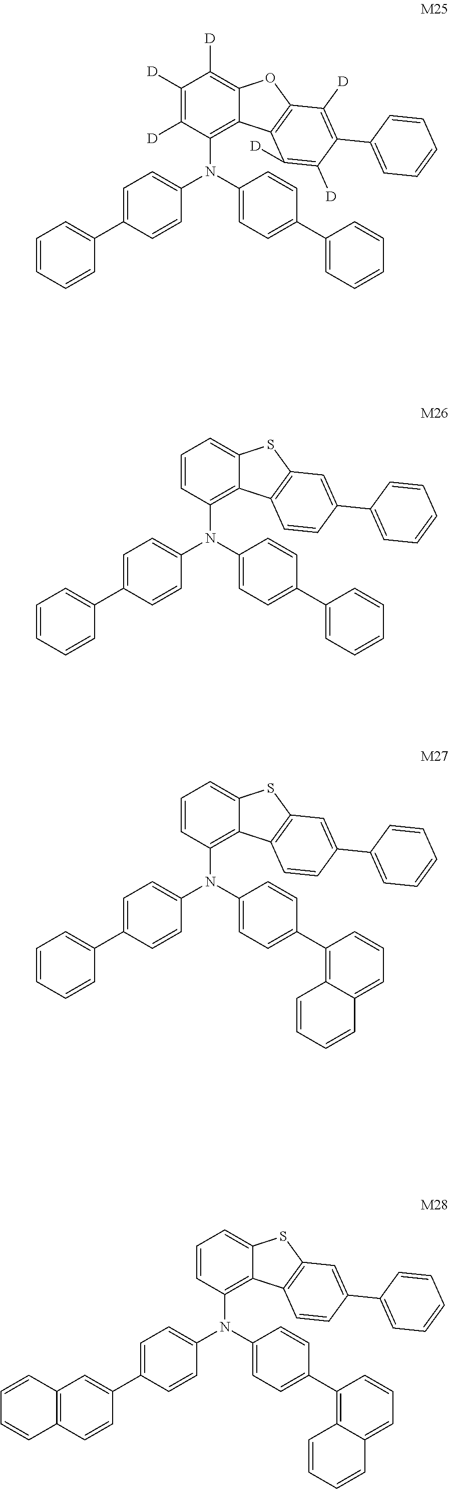 [SMILES: [2H]c1c([2H])c(N(c2ccc(-c3ccccc3)cc2)c2ccc(-c3ccccc3)cc2)c2c(oc3c([2H])c(-c4ccccc4)c([2H])c([2H])c32)c1[2H] ; c1ccc(-c2ccc(N(c3ccc(-c4ccccc4)cc3)c3cccc4sc5cc(-c6ccccc6)ccc5c34)cc2)cc1 ; c1ccc(-c2ccc(N(c3ccc(-c4cccc5ccccc45)cc3)c3cccc4sc5cc(-c6ccccc6)ccc5c34)cc2)cc1 ; c1ccc(-c2ccc3c(c2)sc2cccc(N(c4ccc(-c5ccc6ccccc6c5)cc4)c4ccc(-c5cccc6ccccc56)cc4)c23)cc1]

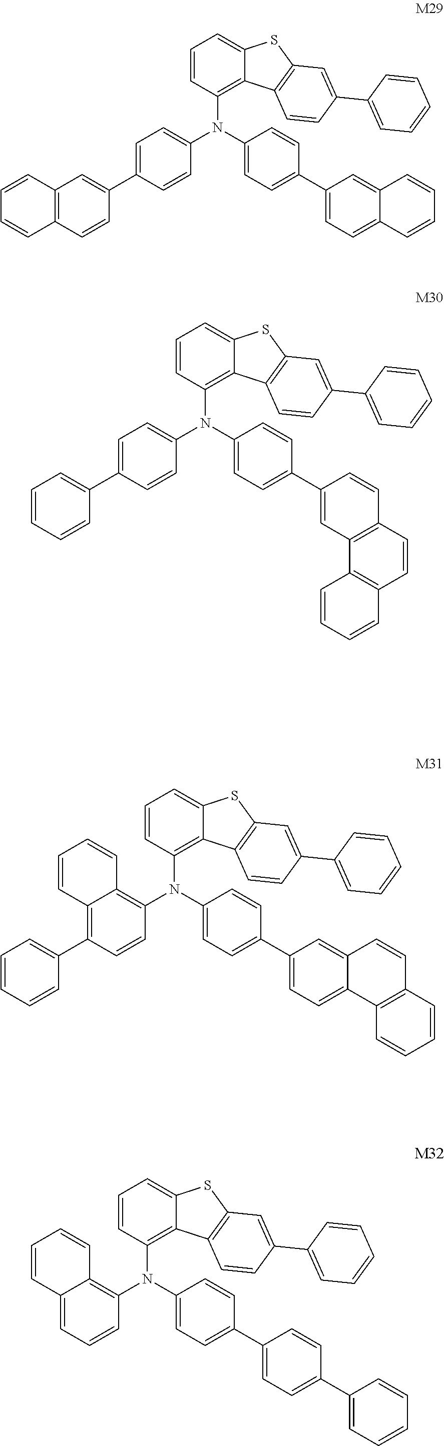 [SMILES: c1ccc(-c2ccc3c(c2)sc2cccc(N(c4ccc(-c5ccc6ccccc6c5)cc4)c4ccc(-c5ccc6ccccc6c5)cc4)c23)cc1 ; c1ccc(-c2ccc(N(c3ccc(-c4ccc5ccc6ccccc6c5c4)cc3)c3cccc4sc5cc(-c6ccccc6)ccc5c34)cc2)cc1 ; c1ccc(-c2ccc3c(c2)sc2cccc(N(c4ccc(-c5ccc6c(ccc7ccccc76)c5)cc4)c4ccc(-c5ccccc5)c5ccccc45)c23)cc1 ; c1ccc(-c2ccc(-c3ccc(N(c4cccc5ccccc45)c4cccc5sc6cc(-c7ccccc7)ccc6c45)cc3)cc2)cc1]

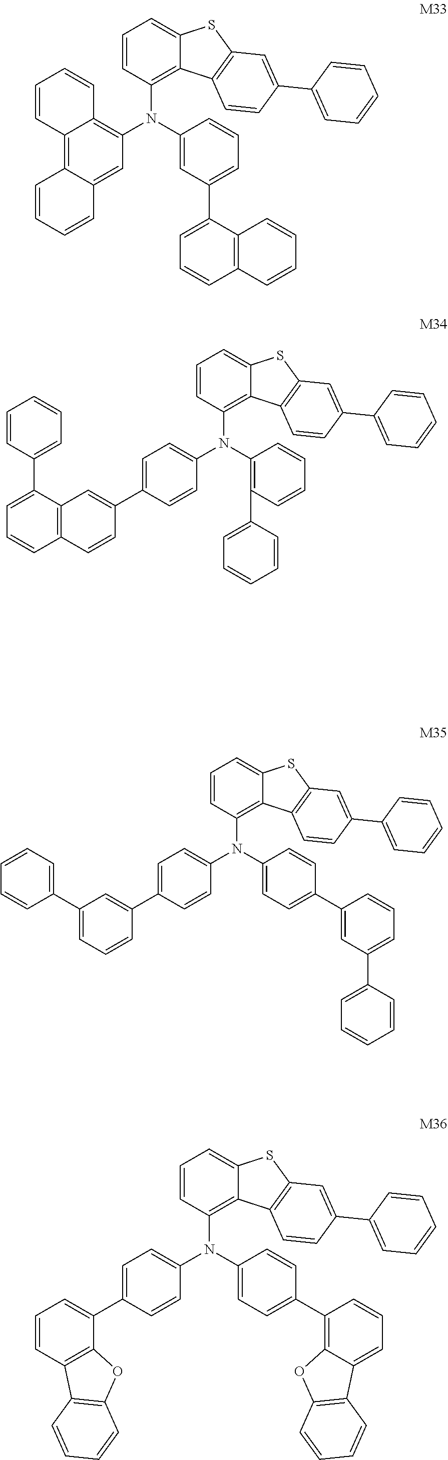 [SMILES: c1ccc(-c2ccc3c(c2)sc2cccc(N(c4cccc(-c5cccc6ccccc56)c4)c4cc5ccccc5c5ccccc45)c23)cc1 ; c1ccc(-c2ccc3c(c2)sc2cccc(N(c4ccc(-c5ccc6cccc(-c7ccccc7)c6c5)cc4)c4ccccc4-c4ccccc4)c23)cc1 ; c1ccc(-c2cccc(-c3ccc(N(c4ccc(-c5cccc(-c6ccccc6)c5)cc4)c4cccc5sc6cc(-c7ccccc7)ccc6c45)cc3)c2)cc1 ; c1ccc(-c2ccc3c(c2)sc2cccc(N(c4ccc(-c5cccc6c5oc5ccccc56)cc4)c4ccc(-c5cccc6c5oc5ccccc56)cc4)c23)cc1]

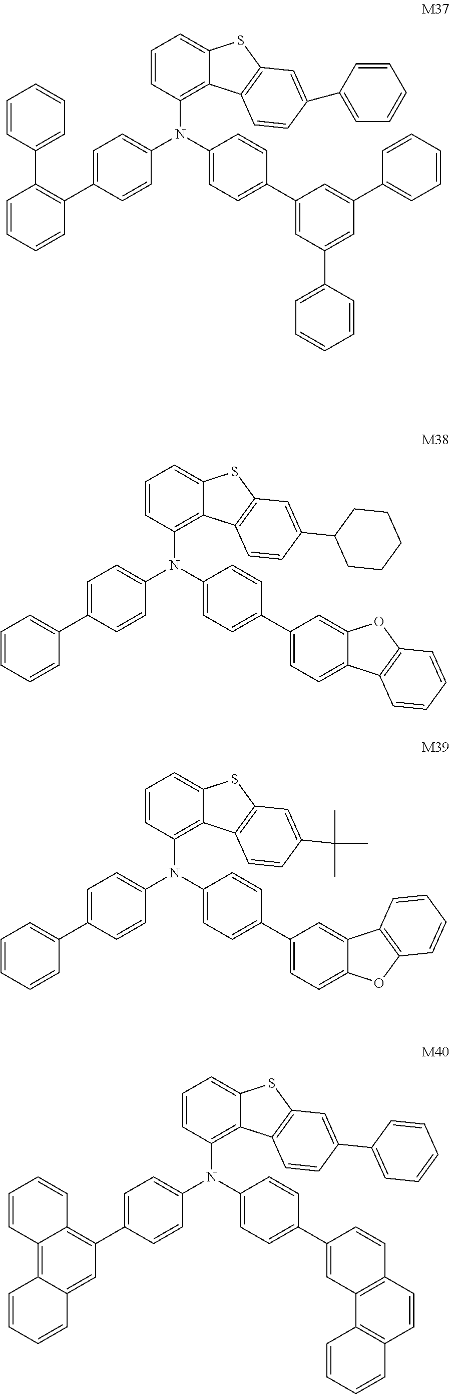 [SMILES: c1ccc(-c2cc(-c3ccccc3)cc(-c3ccc(N(c4ccc(-c5ccccc5-c5ccccc5)cc4)c4cccc5sc6cc(-c7ccccc7)ccc6c45)cc3)c2)cc1 ; c1ccc(-c2ccc(N(c3ccc(-c4ccc5c(c4)oc4ccccc45)cc3)c3cccc4sc5cc(C6CCCCC6)ccc5c34)cc2)cc1 ; CC(C)(C)c1ccc2c(c1)sc1cccc(N(c3ccc(-c4ccccc4)cc3)c3ccc(-c4ccc5oc6ccccc6c5c4)cc3)c12 ; c1ccc(-c2ccc3c(c2)sc2cccc(N(c4ccc(-c5ccc6ccc7ccccc7c6c5)cc4)c4ccc(-c5cc6ccccc6c6ccccc56)cc4)c23)cc1]

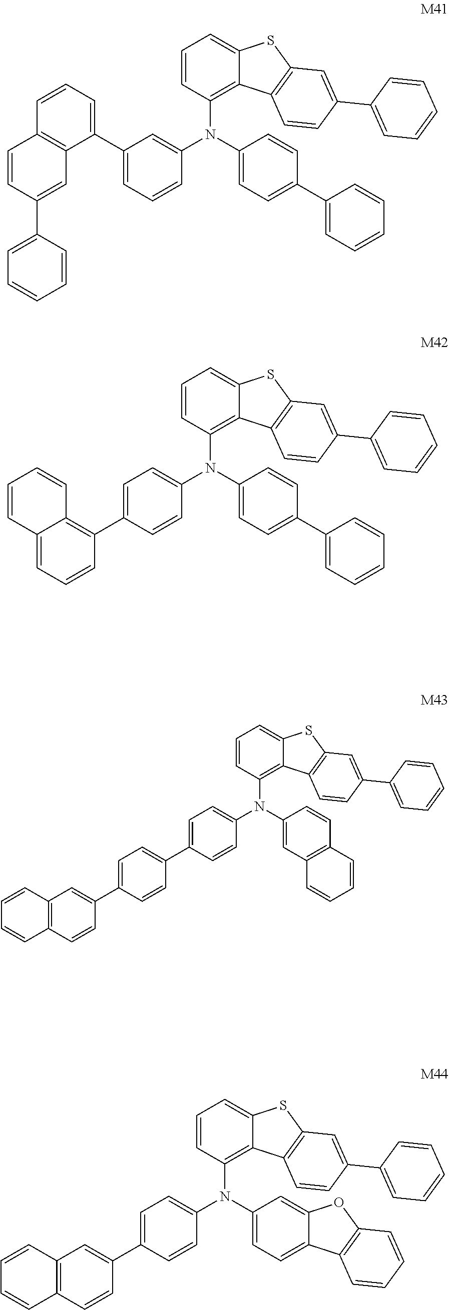 [SMILES: c1ccc(-c2ccc(N(c3cccc(-c4cccc5ccc(-c6ccccc6)cc45)c3)c3cccc4sc5cc(-c6ccccc6)ccc5c34)cc2)cc1 ; c1ccc(-c2ccc(N(c3ccc(-c4cccc5ccccc45)cc3)c3cccc4sc5cc(-c6ccccc6)ccc5c34)cc2)cc1 ; c1ccc(-c2ccc3c(c2)sc2cccc(N(c4ccc(-c5ccc(-c6ccc7ccccc7c6)cc5)cc4)c4ccc5ccccc5c4)c23)cc1 ; c1ccc(-c2ccc3c(c2)sc2cccc(N(c4ccc(-c5ccc6ccccc6c5)cc4)c4ccc5c(c4)oc4ccccc45)c23)cc1]

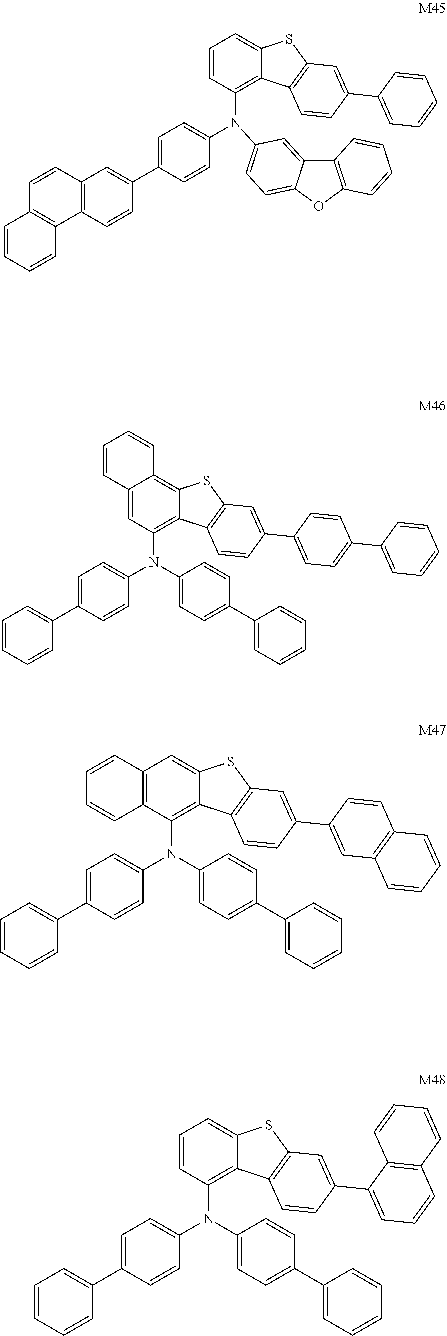 [SMILES: c1ccc(-c2ccc3c(c2)sc2cccc(N(c4ccc(-c5ccc6c(ccc7ccccc76)c5)cc4)c4ccc5oc6ccccc6c5c4)c23)cc1 ; c1ccc(-c2ccc(-c3ccc4c(c3)sc3c5ccccc5cc(N(c5ccc(-c6ccccc6)cc5)c5ccc(-c6ccccc6)cc5)c43)cc2)cc1 ; c1ccc(-c2ccc(N(c3ccc(-c4ccccc4)cc3)c3c4ccccc4cc4sc5cc(-c6ccc7ccccc7c6)ccc5c34)cc2)cc1 ; c1ccc(-c2ccc(N(c3ccc(-c4ccccc4)cc3)c3cccc4sc5cc(-c6cccc7ccccc67)ccc5c34)cc2)cc1]

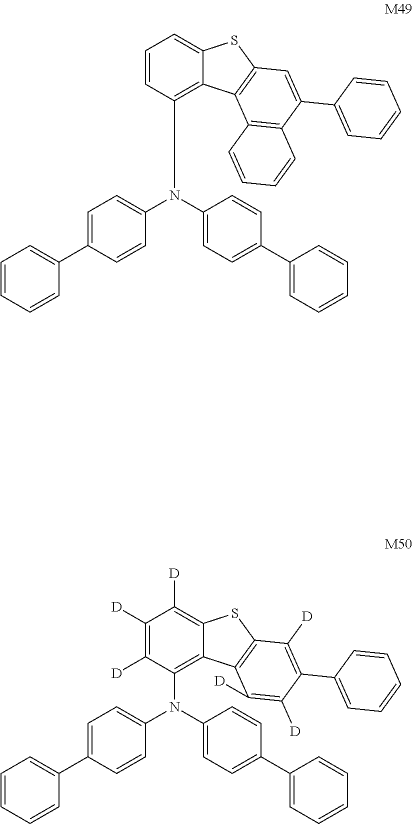 [SMILES: c1ccc(-c2ccc(N(c3ccc(-c4ccccc4)cc3)c3cccc4sc5cc(-c6ccccc6)c6ccccc6c5c34)cc2)cc1 ; [2H]c1c([2H])c(N(c2ccc(-c3ccccc3)cc2)c2ccc(-c3ccccc3)cc2)c2c(sc3c([2H])c(-c4ccccc4)c([2H])c([2H])c32)c1[2H]]

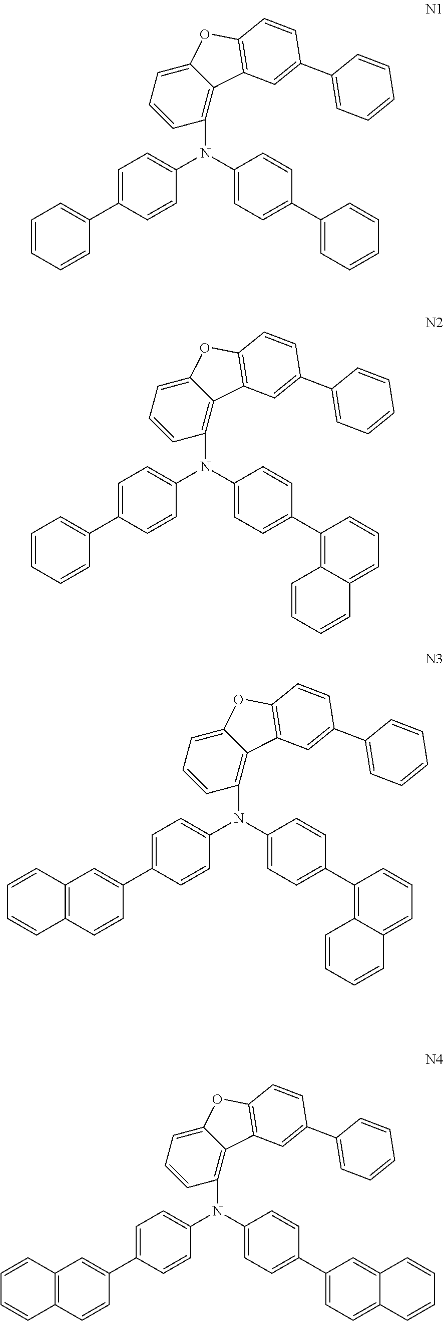 [SMILES: c1ccc(-c2ccc(N(c3ccc(-c4ccccc4)cc3)c3cccc4oc5ccc(-c6ccccc6)cc5c34)cc2)cc1 ; c1ccc(-c2ccc(N(c3ccc(-c4cccc5ccccc45)cc3)c3cccc4oc5ccc(-c6ccccc6)cc5c34)cc2)cc1 ; c1ccc(-c2ccc3oc4cccc(N(c5ccc(-c6ccc7ccccc7c6)cc5)c5ccc(-c6cccc7ccccc67)cc5)c4c3c2)cc1 ; c1ccc(-c2ccc3oc4cccc(N(c5ccc(-c6ccc7ccccc7c6)cc5)c5ccc(-c6ccc7ccccc7c6)cc5)c4c3c2)cc1]

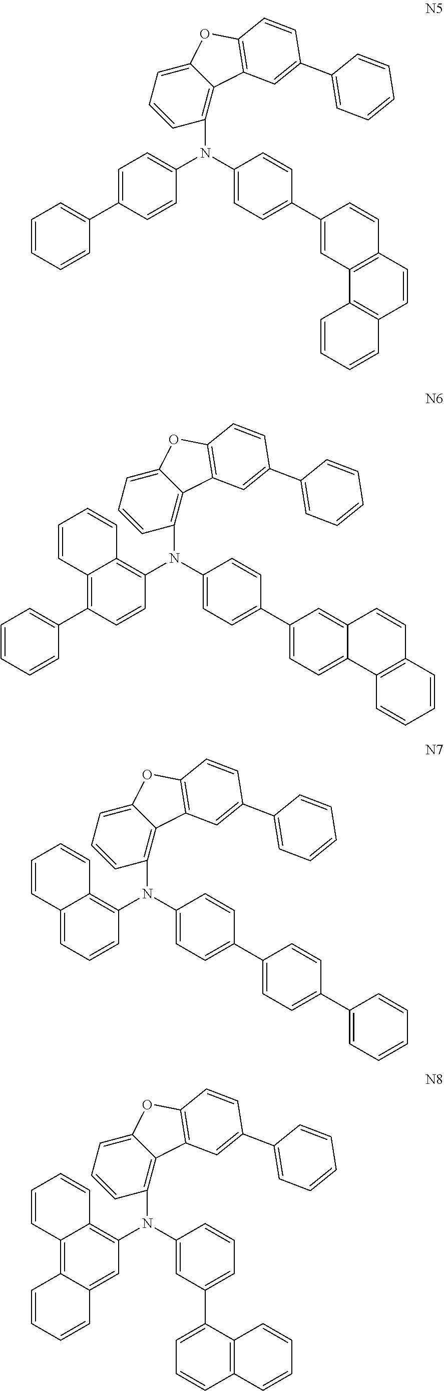 [SMILES: c1ccc(-c2ccc(N(c3ccc(-c4ccc5ccc6ccccc6c5c4)cc3)c3cccc4oc5ccc(-c6ccccc6)cc5c34)cc2)cc1 ; c1ccc(-c2ccc3oc4cccc(N(c5ccc(-c6ccc7c(ccc8ccccc87)c6)cc5)c5ccc(-c6ccccc6)c6ccccc56)c4c3c2)cc1 ; c1ccc(-c2ccc(-c3ccc(N(c4cccc5ccccc45)c4cccc5oc6ccc(-c7ccccc7)cc6c45)cc3)cc2)cc1 ; c1ccc(-c2ccc3oc4cccc(N(c5cccc(-c6cccc7ccccc67)c5)c5cc6ccccc6c6ccccc56)c4c3c2)cc1]

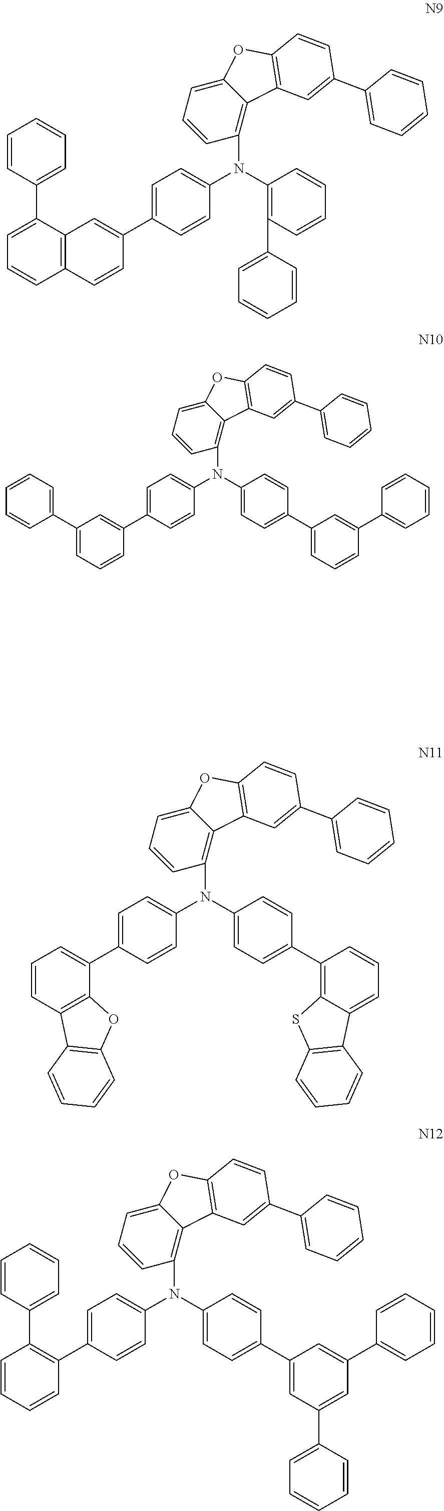

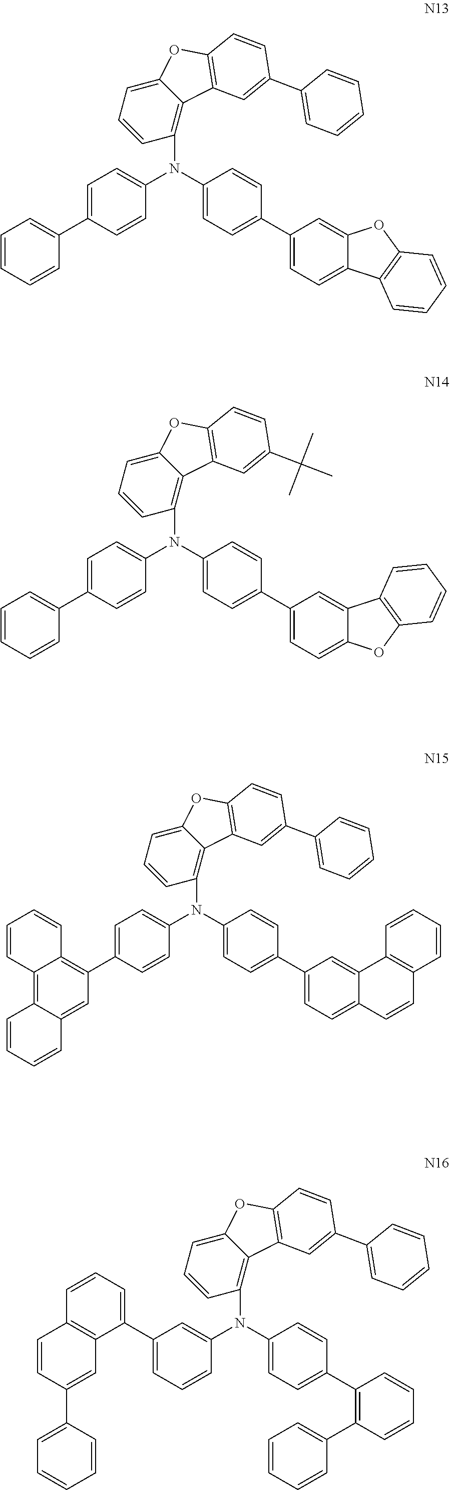 [SMILES: c1ccc(-c2ccc(N(c3ccc(-c4ccc5c(c4)oc4ccccc45)cc3)c3cccc4oc5ccc(-c6ccccc6)cc5c34)cc2)cc1 ; CC(C)(C)c1ccc2oc3cccc(N(c4ccc(-c5ccccc5)cc4)c4ccc(-c5ccc6oc7ccccc7c6c5)cc4)c3c2c1 ; c1ccc(-c2ccc3oc4cccc(N(c5ccc(-c6ccc7ccc8ccccc8c7c6)cc5)c5ccc(-c6cc7ccccc7c7ccccc67)cc5)c4c3c2)cc1 ; c1ccc(-c2ccc3cccc(-c4cccc(N(c5ccc(-c6ccccc6-c6ccccc6)cc5)c5cccc6oc7ccc(-c8ccccc8)cc7c56)c4)c3c2)cc1]

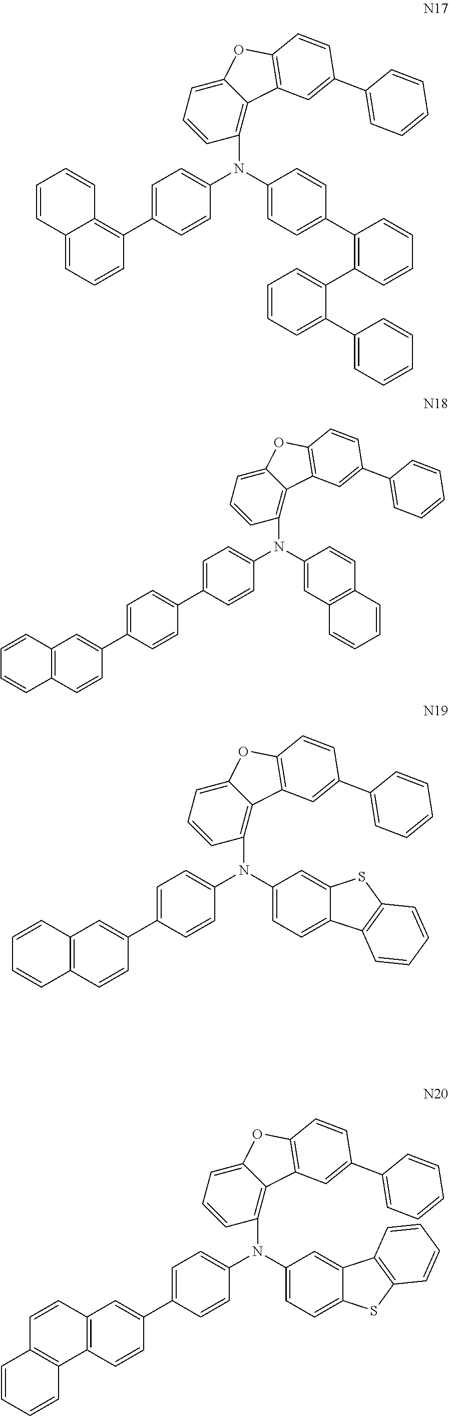 [SMILES: c1ccc(-c2ccc3oc4cccc(N(c5ccc(-c6ccccc6-c6ccccc6-c6ccccc6)cc5)c5ccc(-c6cccc7ccccc67)cc5)c4c3c2)cc1 ; c1ccc(-c2ccc3oc4cccc(N(c5ccc(-c6ccc(-c7ccc8ccccc8c7)cc6)cc5)c5ccc6ccccc6c5)c4c3c2)cc1 ; c1ccc(-c2ccc3oc4cccc(N(c5ccc(-c6ccc7ccccc7c6)cc5)c5ccc6c(c5)sc5ccccc56)c4c3c2)cc1 ; c1ccc(-c2ccc3oc4cccc(N(c5ccc(-c6ccc7c(ccc8ccccc87)c6)cc5)c5ccc6sc7ccccc7c6c5)c4c3c2)cc1]

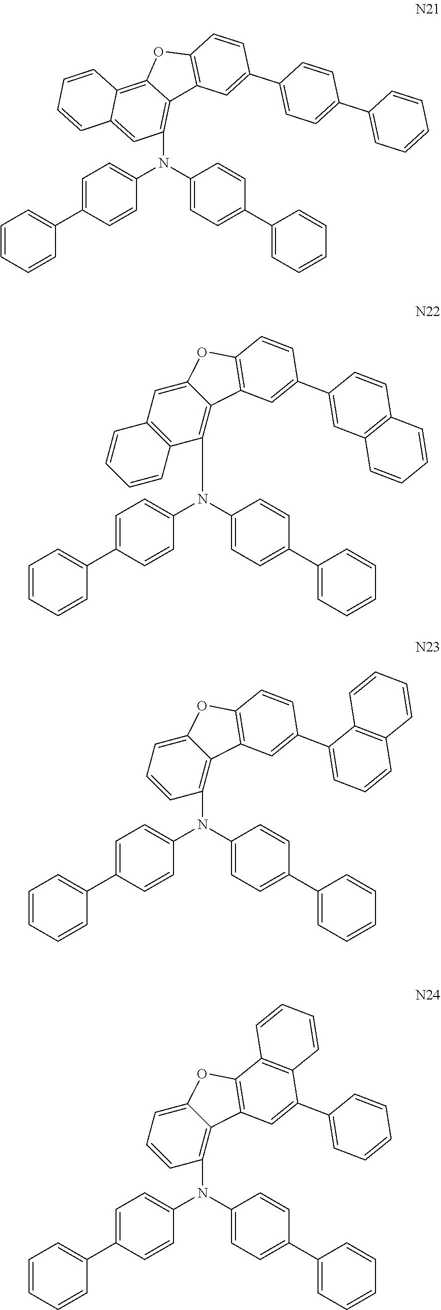

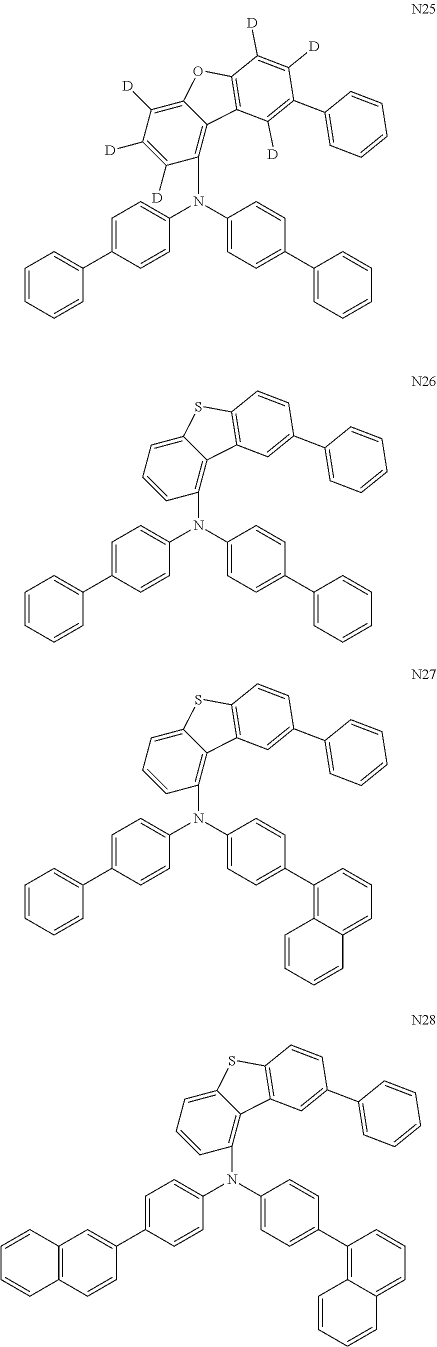 [SMILES: [2H]c1c(-c2ccccc2)c([2H])c2c(oc3c([2H])c([2H])c([2H])c(N(c4ccc(-c5ccccc5)cc4)c4ccc(-c5ccccc5)cc4)c32)c1[2H] ; c1ccc(-c2ccc(N(c3ccc(-c4ccccc4)cc3)c3cccc4sc5ccc(-c6ccccc6)cc5c34)cc2)cc1 ; c1ccc(-c2ccc(N(c3ccc(-c4cccc5ccccc45)cc3)c3cccc4sc5ccc(-c6ccccc6)cc5c34)cc2)cc1 ; c1ccc(-c2ccc3sc4cccc(N(c5ccc(-c6ccc7ccccc7c6)cc5)c5ccc(-c6cccc7ccccc67)cc5)c4c3c2)cc1]

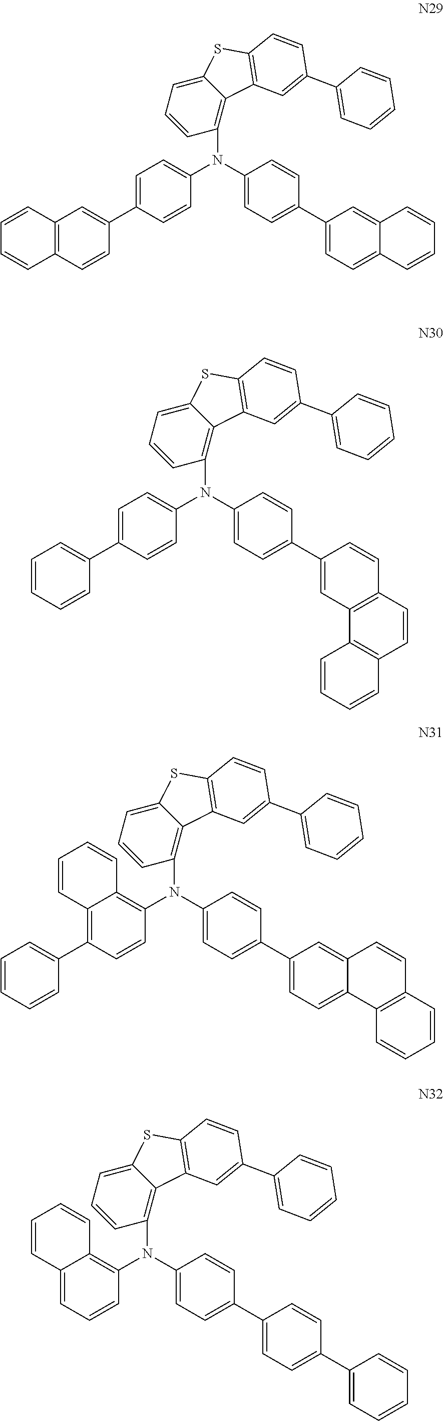 [SMILES: c1ccc(-c2ccc3sc4cccc(N(c5ccc(-c6ccc7ccccc7c6)cc5)c5ccc(-c6ccc7ccccc7c6)cc5)c4c3c2)cc1 ; c1ccc(-c2ccc(N(c3ccc(-c4ccc5ccc6ccccc6c5c4)cc3)c3cccc4sc5ccc(-c6ccccc6)cc5c34)cc2)cc1 ; c1ccc(-c2ccc3sc4cccc(N(c5ccc(-c6ccc7c(ccc8ccccc87)c6)cc5)c5ccc(-c6ccccc6)c6ccccc56)c4c3c2)cc1 ; c1ccc(-c2ccc(-c3ccc(N(c4cccc5ccccc45)c4cccc5sc6ccc(-c7ccccc7)cc6c45)cc3)cc2)cc1]

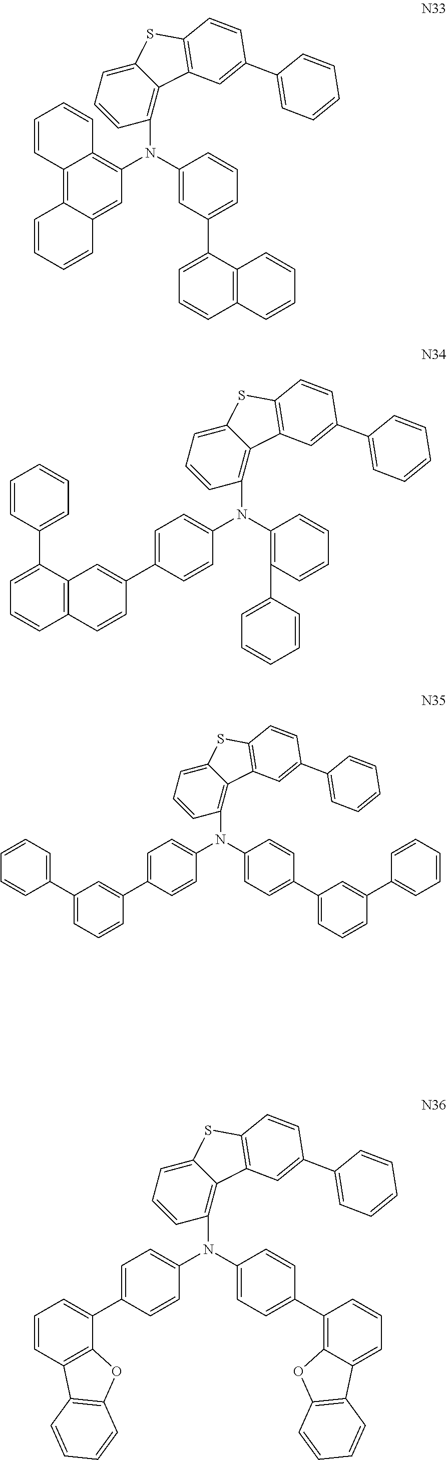 [SMILES: c1ccc(-c2ccc3sc4cccc(N(c5cccc(-c6cccc7ccccc67)c5)c5cc6ccccc6c6ccccc56)c4c3c2)cc1 ; c1ccc(-c2ccc3sc4cccc(N(c5ccc(-c6ccc7cccc(-c8ccccc8)c7c6)cc5)c5ccccc5-c5ccccc5)c4c3c2)cc1 ; c1ccc(-c2cccc(-c3ccc(N(c4ccc(-c5cccc(-c6ccccc6)c5)cc4)c4cccc5sc6ccc(-c7ccccc7)cc6c45)cc3)c2)cc1 ; c1ccc(-c2ccc3sc4cccc(N(c5ccc(-c6cccc7c6oc6ccccc67)cc5)c5ccc(-c6cccc7c6oc6ccccc67)cc5)c4c3c2)cc1]

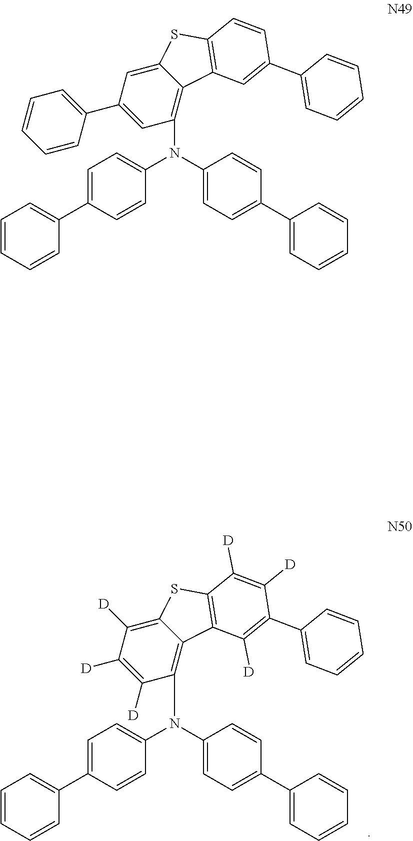 [SMILES: c1ccc(-c2ccc(N(c3ccc(-c4ccccc4)cc3)c3cc(-c4ccccc4)cc4sc5ccc(-c6ccccc6)cc5c34)cc2)cc1 ; [2H]c1c(-c2ccccc2)c([2H])c2c(sc3c([2H])c([2H])c([2H])c(N(c4ccc(-c5ccccc5)cc4)c4ccc(-c5ccccc5)cc4)c32)c1[2H]]

Referring to FIG. 3 to FIG. 6 again, the luminescence device ED according to embodiments of the present disclosure will be explained.

As described above, the hole transport region HTR includes the aforementioned amine compound according to embodiments of the present disclosure. For example, the hole transport region HTR includes the amine compound represented by Formula 1.

When the hole transport region HTR has a multilayer structure having multiple layers, any one layer among the multiple layers may include the amine compound represented by Formula 1. For example, a hole transport region HTR may include a hole injection layer HIL disposed on a first electrode EL1 and a hole transport layer HTL disposed on the hole injection layer HIL, and the hole transport layer HTL may include the amine compound represented by Formula 1. However, embodiments of the present disclosure are not limited thereto, and for example, the hole injection layer HIL may include the amine compound represented by Formula 1.

The hole transport region HTR may include one or two or more types (kinds) of the amine compound represented by Formula 1. For example, the hole transport region HTR may include at least one selected from the compounds represented in Compound Groups 1 to 3.

The hole transport region HTR may be formed utilizing various suitable methods (such as a vacuum deposition method, a spin coating method, a cast method, a Langmuir-Blodgett (LB) method, an inkjet printing method, a laser printing method, and/or a laser induced thermal imaging (LITI) method).

The hole transport region HTR may include a compound represented by Formula H-1.

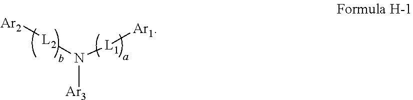

In Formula H-1, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. “a” and “b” may each independently be an integer of 0 to 10. In some embodiments, when “a” or “b” is an integer of 2 or more, multiple L₁ and L₂ may each independently be a substituted or unsubstituted arylene group of 6 to 30 ring-forming carbon atoms, or a substituted or unsubstituted heteroarylene group of 2 to 30 ring-forming carbon atoms.

In Formula H-1, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. In Formula H-1, Ar₃ may be a substituted or unsubstituted aryl group of 6 to 30 ring-forming carbon atoms, or a substituted or unsubstituted heteroaryl group of 2 to 30 ring-forming carbon atoms.

The compound represented by Formula H-1 may be a monoamine compound. In some embodiments, the compound represented by Formula H-1 may be a diamine compound in which at least one selected from among Ar₁ to Ar₃ includes an amine group as a substituent. In some embodiments, the compound represented by Formula H-1 may be a carbazole-based compound in which at least one selected from among Ar₁ to Ar₃ includes a substituted or unsubstituted carbazole group, or a fluorene-based compound in which at least one selected from among Ar₁ to Ar₃ includes a substituted or unsubstituted fluorene group.

The compound represented by Formula H-1 may be represented by any one selected from among the compounds in Compound Group H. However, the compounds shown in Compound Group H are only illustrations, and the compound represented by Formula H-1 is not limited to the compounds represented in Compound Group H.

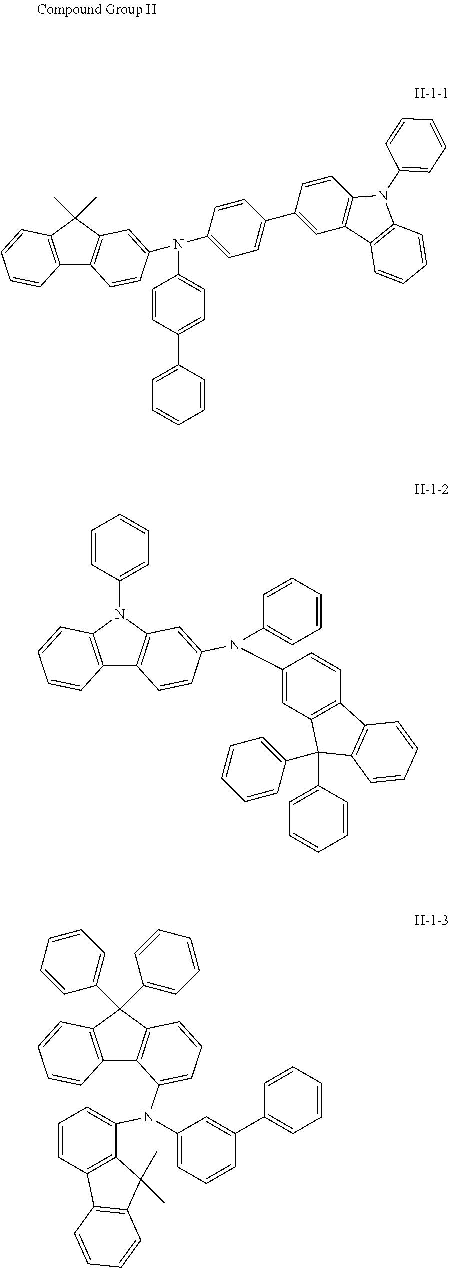 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

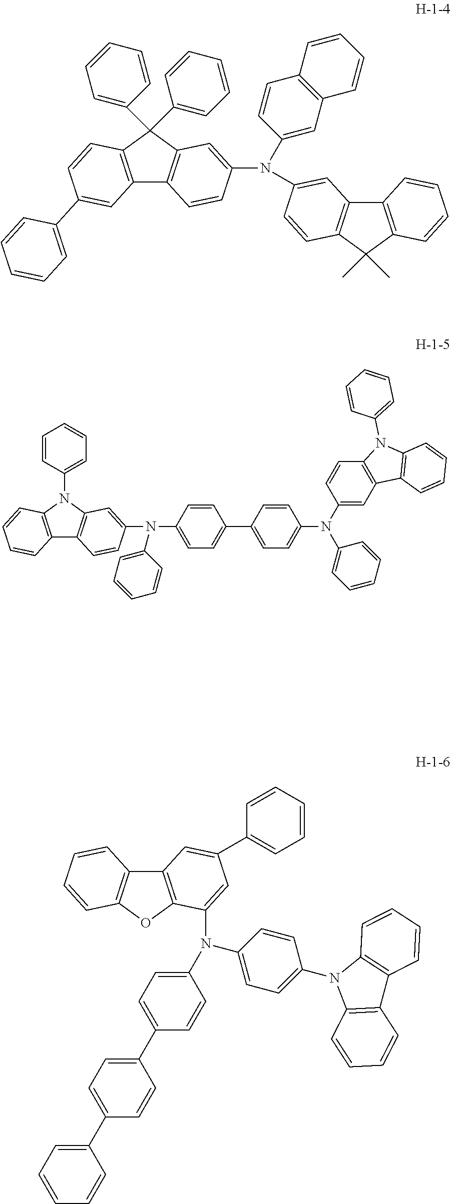 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

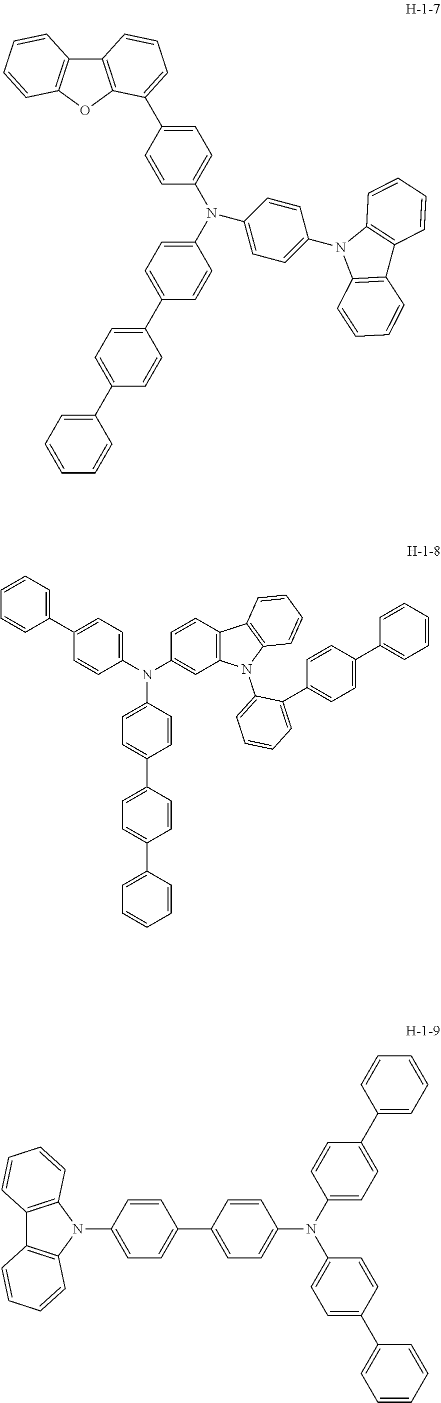 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

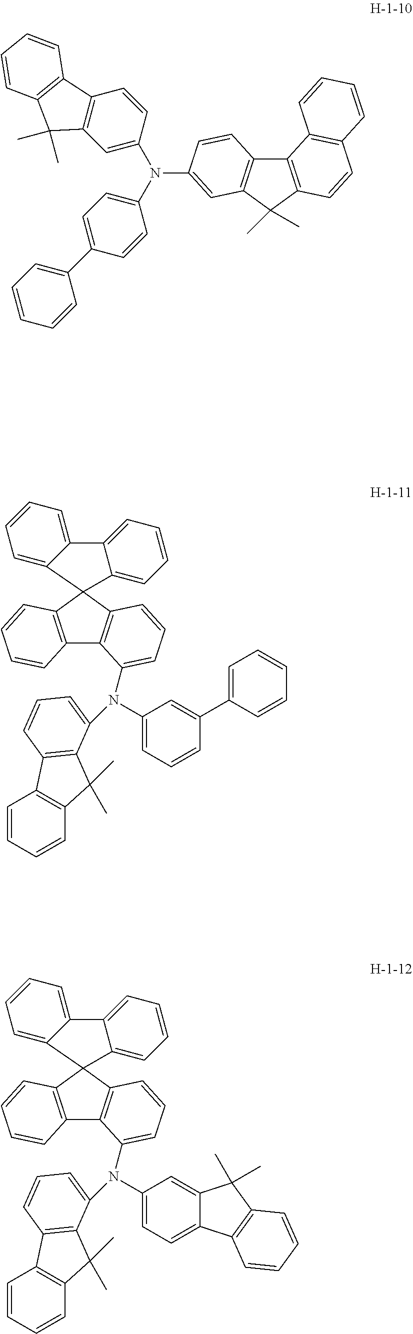 [SMILES: CC1(C)c2ccccc2-c2ccc(N(c3ccc(-c4ccccc4)cc3)c3ccc4c(c3)C(C)(C)c3ccc5ccccc5c3-4)cc21 ; CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21]

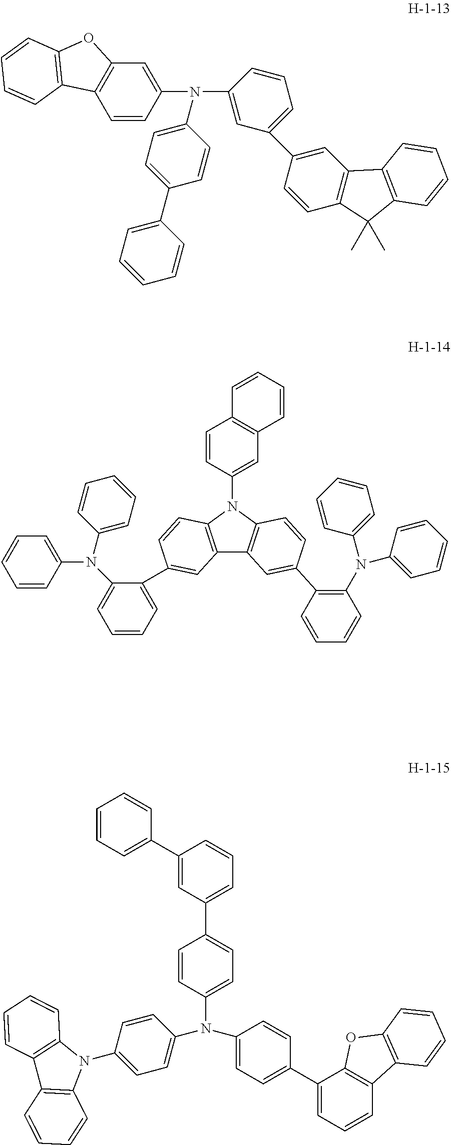 [SMILES: CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1]

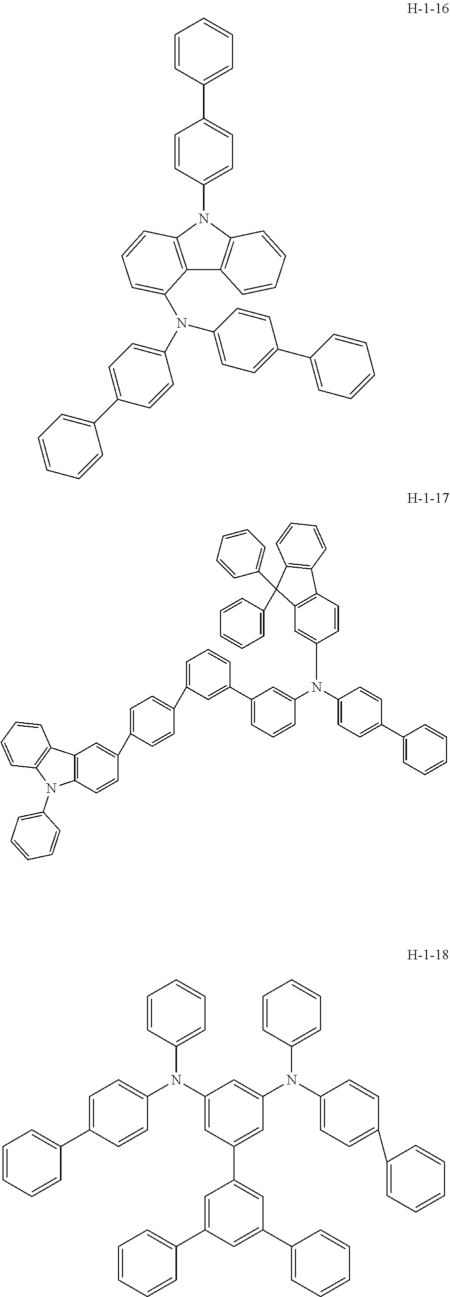 [SMILES: c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc(-c6ccc7c(c6)c6ccccc6n7-c6ccccc6)cc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1]

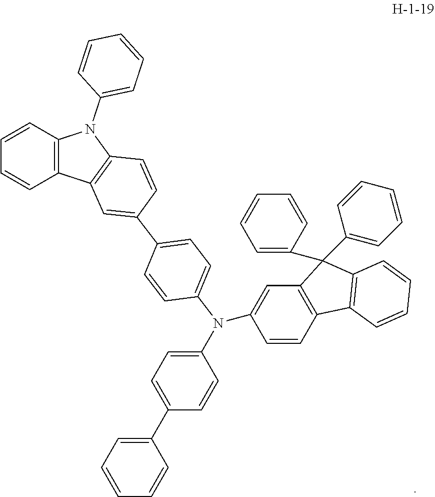 [SMILES: c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound (such as copper phthalocyanine), N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylene dioxythiophene)/poly(4-styrene sulfonate) (PEDOT/PSS), polyaniline/dodecylbenzene sulfonic acid (PANI/DBSA), polyaniline/camphor sulfonic acid (PANI/CSA), polyaniline/poly(4-styrene sulfonate) (PANI/PSS), N,N′-di(1-naphthalene-1-yl)-N,N′-diphenyl-benzidine (NPB), triphenylamine-containing polyether ketone (TPAPEK), 4-isopropyl-4′-methyldiphenyliodonium [tetrakis(pentafluorophenyl)borate], and dipyrazino[2,3-f:2′,3′-h] quinoxaline-2,3,6,7,10,11-hexacarbonitrile (HAT-CN).

The hole transport region HTR may include, for example, carbazole derivatives (such as N-phenyl carbazole and/or polyvinyl carbazole), fluorene-based derivatives, N,N′-bis(3-methylphenyl)-N,N′-diphenyl-[1,1′-biphenyl]-4,4′-diamine (TPD), triphenylamine-based derivatives (such as 4,4′,4″-tris(N-carbazolyl)triphenylamine (TCTA)), N,N′-di(1-naphthalene-1-yl)-N,N′-diphenyl-benzidine (NPB), 4,4′-cyclohexylidene bis[N,N-bis(4-methylphenyl)benzeneamine (TAPC), 4,4′-bis[N,N′-(3-tolyl)amino]-3,3′-dimethylbiphenyl (HMTPD), 1,3-bis(N-carbazolyl)benzene (mCP), etc.

The hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the compounds of the hole transport region in at least one selected from among the hole injection layer HIL, hole transport layer HTL, and electron blocking layer EBL.

The thickness of the hole transport region HTR may be about 100 Å to about 10,000 Å, for example, about 100 Å to about 5,000 Å. The thickness of the hole injection region HIL may be, for example, about 30 Å to about 1,000 Å. The thickness of the hole transport layer HTL may be about 30 Å to about 1,000 Å. For example, the thickness of the electron blocking layer EBL may be about 10 Å to about 1,000 Å. When the thicknesses of the hole transport region HTR, the hole injection layer HIL, the hole transport layer HTL and the electron blocking layer EBL satisfy the above-described ranges, satisfactory hole transport properties may be achieved without substantial increase of a driving voltage.

The hole transport region HTR may further include a charge generating material to increase conductivity in addition to the above-described materials. The charge generating material may be dispersed substantially uniformly or non-uniformly in the hole transport region HTR. The charge generating material may be, for example, a p-dopant. The p-dopant may include any one selected from among quinone derivatives, metal oxides, and cyano group-containing compounds, without limitation. For example, non-limiting examples of the p-dopant may include quinone derivatives (such as tetracyanoquinodimethane (TCNQ) and/or 2,3,5,6-tetrafluoro-7,7′,8,8-tetracyanoquinodimethane (F4-TCNQ)), metal oxides (such as tungsten oxide and/or molybdenum oxide), etc., without limitation.

As described above, the hole transport region HTR may further include at least one of a hole buffer layer or an electron blocking layer EBL in addition to the hole injection layer HIL and the hole transport layer HTL. The hole buffer layer may compensate for a resonance distance of the wavelength of light emitted from an emission layer EML, and may thereby increase the light emitting efficiency of the device. As materials included in the hole buffer layer, materials that may be included in the hole transport region HTR may be utilized. The electron blocking layer EBL may block or reduce the injection of electrons from an electron transport region ETR to a hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed utilizing a single material, a single layer formed utilizing multiple different materials, or a multilayer structure having multiple layers formed utilizing multiple different materials.

In the luminescence device ED of an embodiment, the emission layer EML may include anthracene derivatives, pyrene derivatives, fluoranthene derivatives, chrysene derivatives, dihydrobenzanthracene derivatives, and/or triphenylene derivatives. For example, the emission layer EML may further include anthracene derivatives and/or pyrene derivatives.

In the luminescence devices ED of embodiments, shown in FIG. 3 to FIG. 6, the emission layer EML may include a host and a dopant, and the emission layer EML may include a compound represented by Formula E-1. The compound represented by Formula E-1 may be utilized as a fluorescence host material.

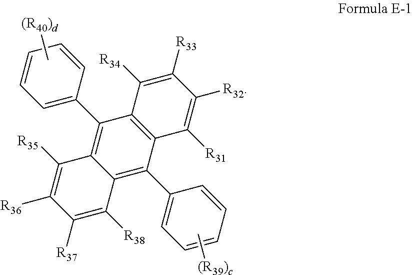

In Formula E-1, R₃₁ to R₄₀ may each independently be a hydrogen atom, a deuterium atom, a halogen atom, a substituted or unsubstituted silyl group, a substituted or unsubstituted alkyl group of 1 to 10 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring. In some embodiments, R₃₁ to R₄₀ may be combined with an adjacent group to form a saturated hydrocarbon ring or an unsaturated hydrocarbon ring.

In Formula E-1, “c” and “d” may be an integer of 0 to 5.

Formula E-1 may be represented by any one selected from among Compound E1 to Compound E19.

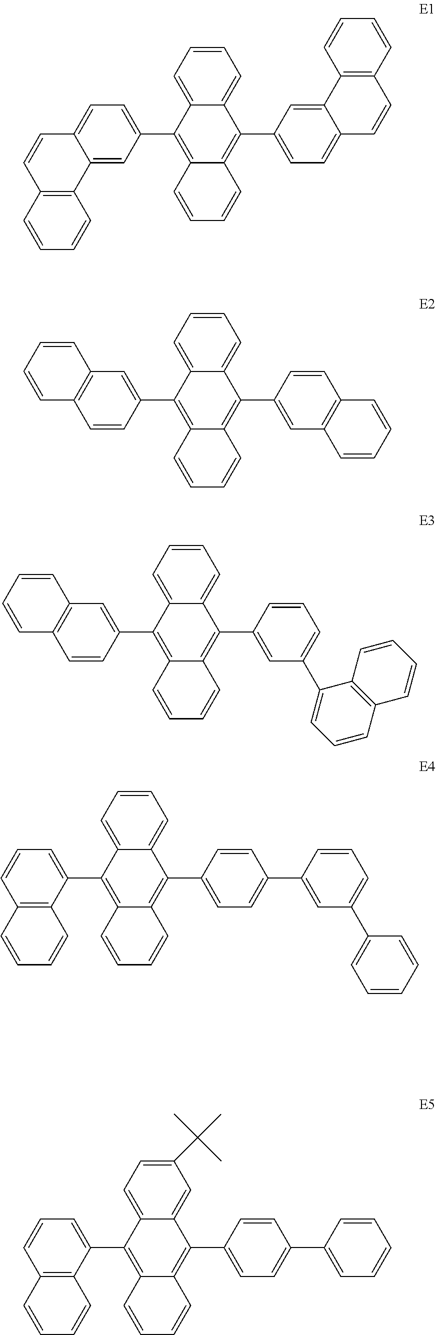

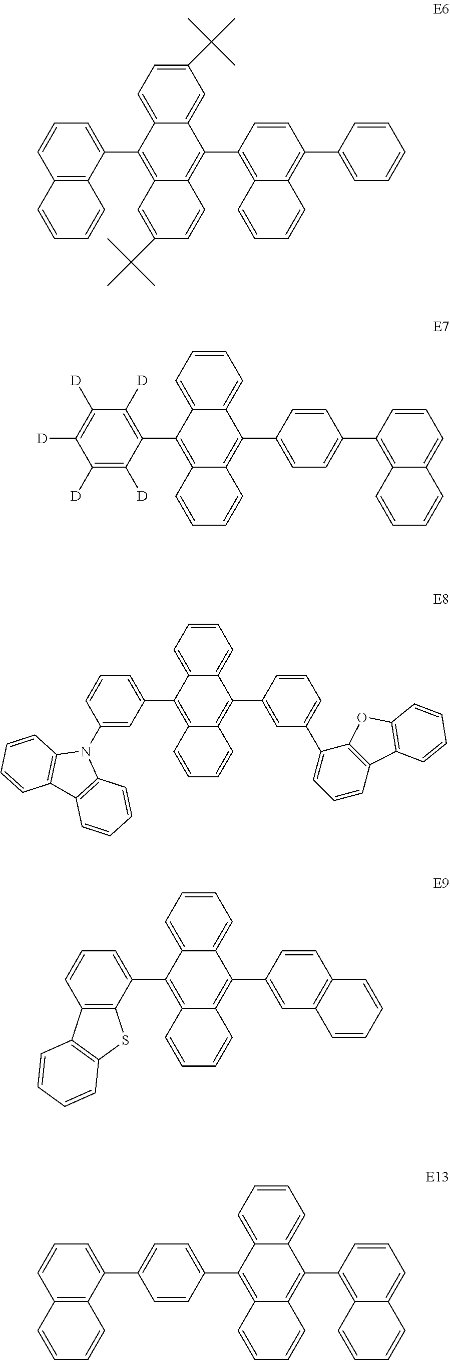

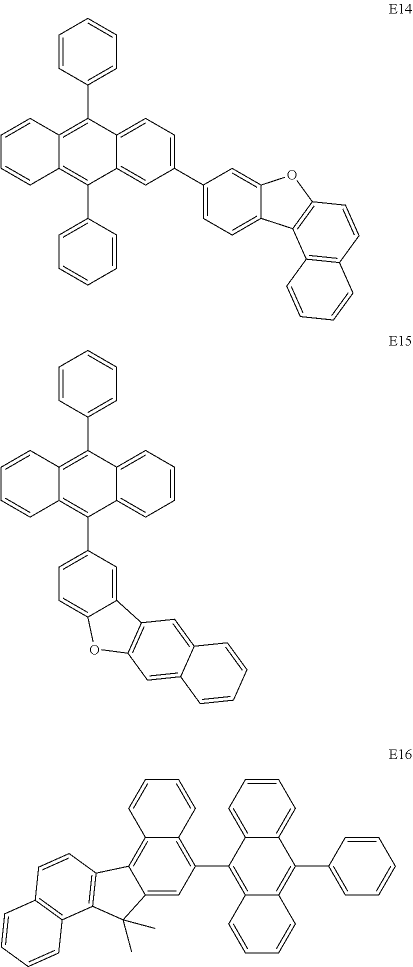

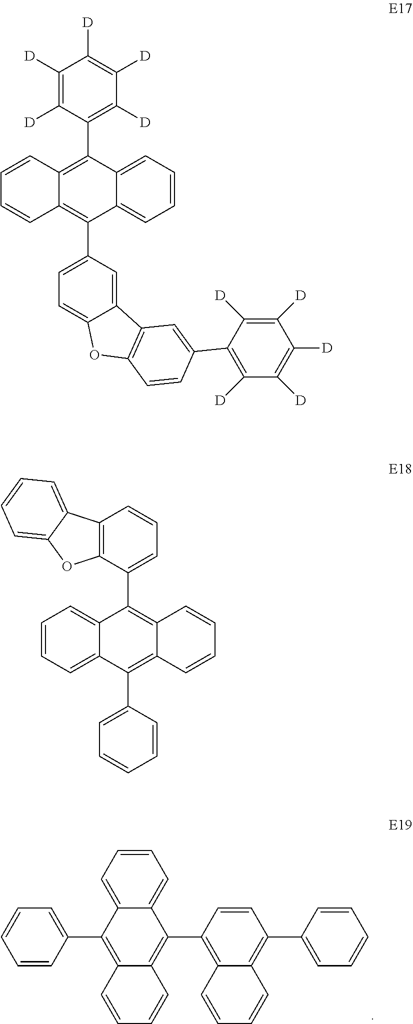

In an embodiment, the emission layer EML may include a compound represented by Formula E-2a or Formula E-2b. The compound represented by Formula E-2a or Formula E-2b may be utilized as a phosphorescence host material.

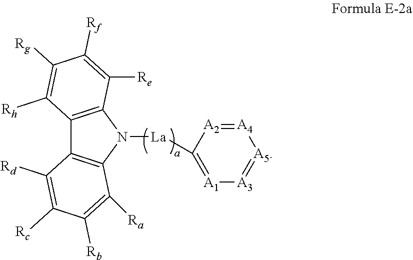

In Formula E-2b, “a” may be an integer of 0 to 10, and Lₐ may be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” is an integer of 2 or more, multiple Lₐ may each independently be a substituted or unsubstituted arylene group of 6 to 30 ring-forming carbon atoms, or a substituted or unsubstituted heteroarylene group of 2 to 30 ring-forming carbon atoms.

In some embodiments, in Formula E-2a, A₁ to A5 may each independently be N or CRi. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Rₐ to Rᵢ may be combined with an adjacent group to form a hydrocarbon ring or a heterocycle including N, O, S, etc. as a ring-forming atom.

In some embodiments, in Formula E-2a, two or three selected from A₁ to A₅ may be N, and the remainder may be CRᵢ.

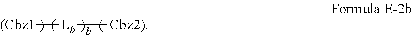

In Formula E-2b, Cbz1 and Cbz2 may each independently be an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” may be an integer of 0 to 10, and when “b” is an integer of 2 or more, multiple L_{b} may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one selected from among the compounds in Compound Group E-2. However, the compounds shown in Compound Group E-2 are only illustrations, and the compound represented by Formula E-2a or Formula E-2b is not limited to the compounds represented in Compound Group E-2.

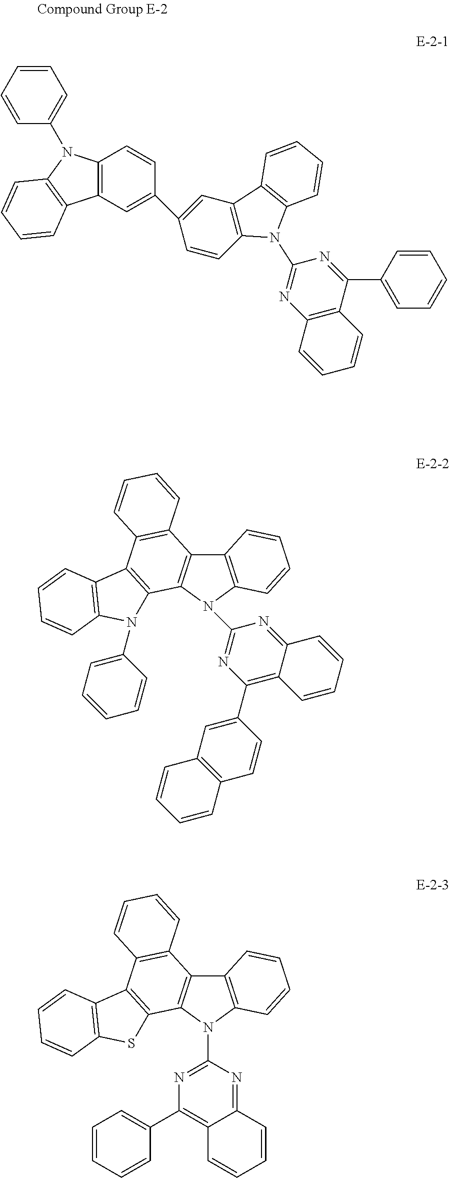 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

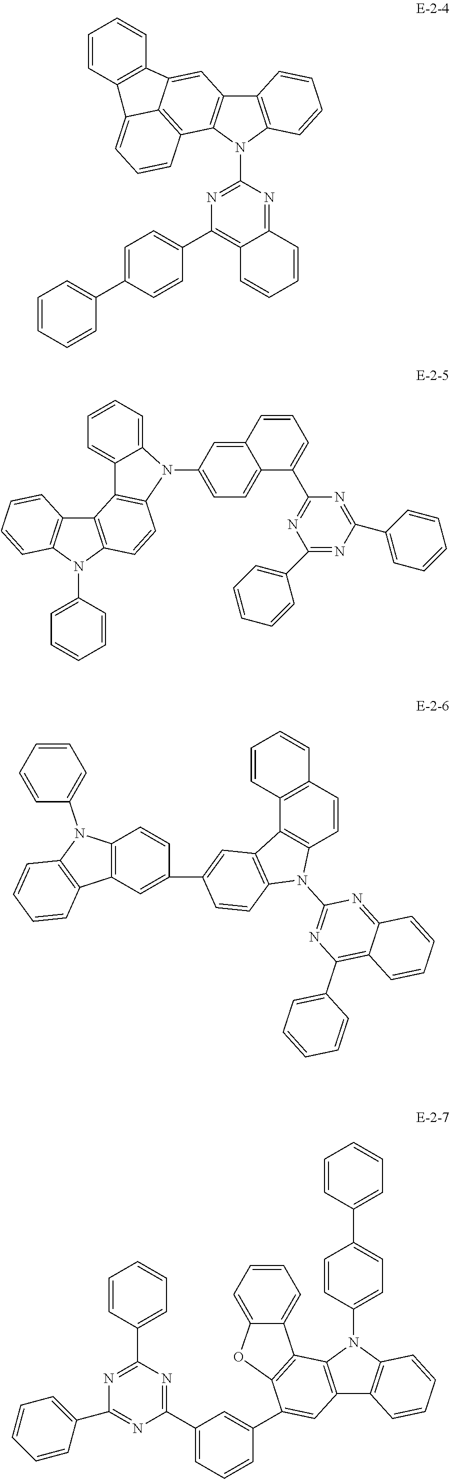 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1]

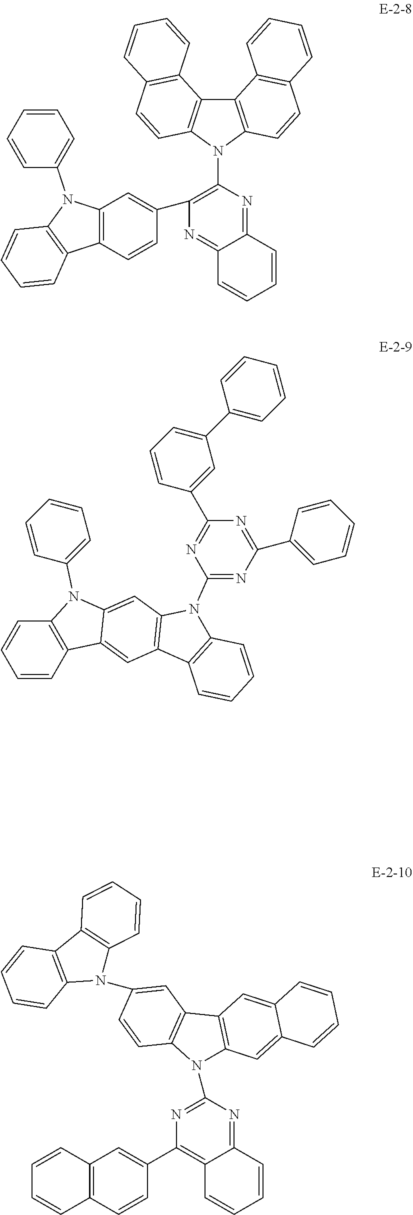 [SMILES: c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

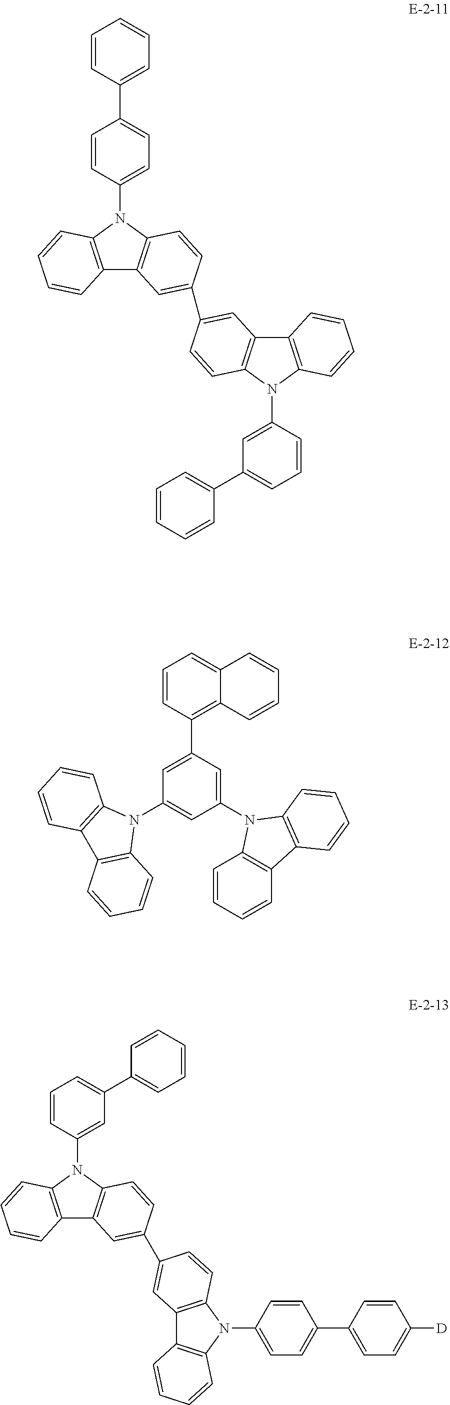 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1]

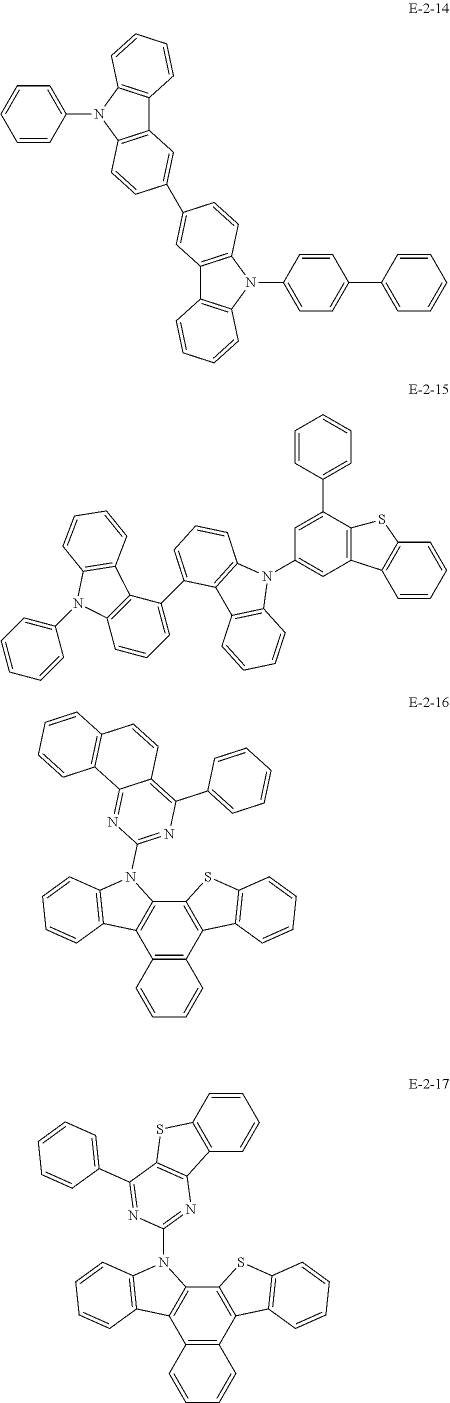

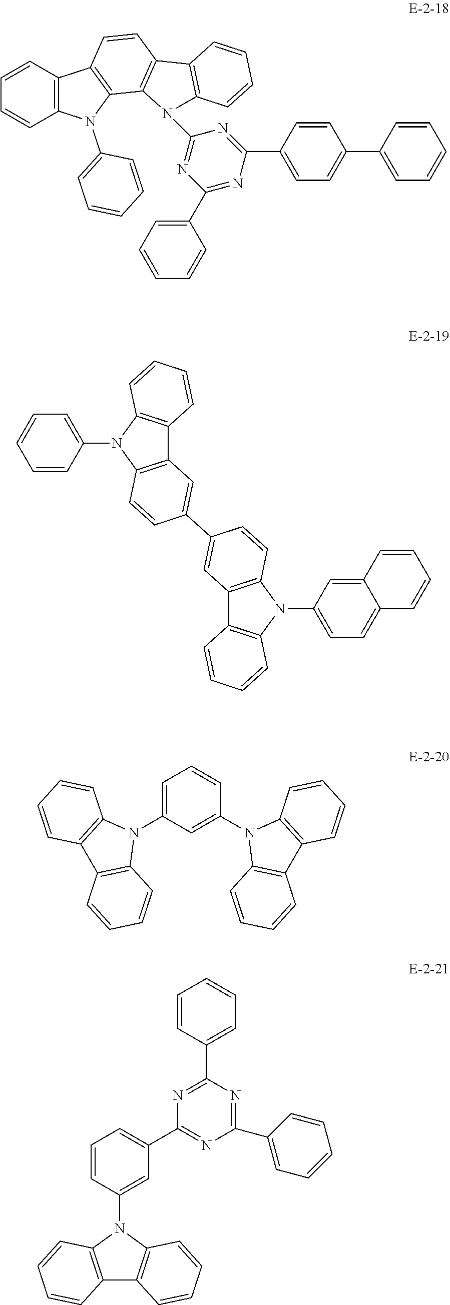 [SMILES: c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

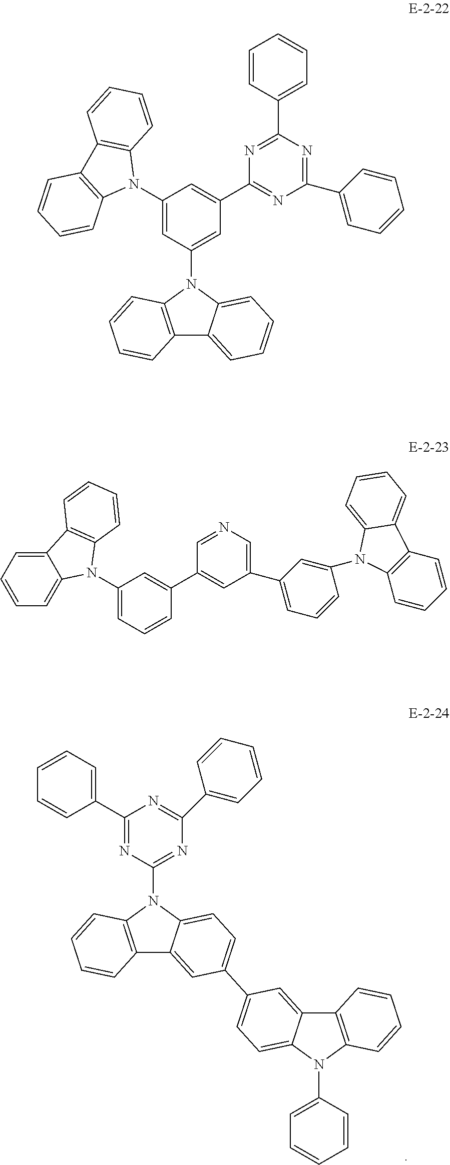 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a common material well-known in the art as a host material. For example, the emission layer EML may include as a host material, at least one of bis[2-(diphenylphosphino)phenyl] ether oxide (DPEPO), 4,4′-bis(carbazol-9-yl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, embodiments of the present disclosure are not limited thereto. For example, tris(8-hydroxyquinolino)aluminum (Alq3), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be utilized as the host material.

The emission layer EML may include a compound represented by Formula M-a or Formula M-b. The compound represented by Formula M-a or Formula M-b may be utilized as a phosphorescence dopant material.

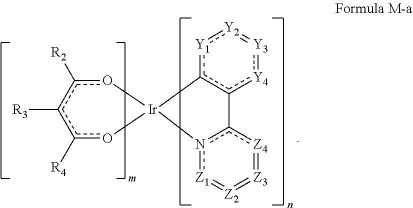

In Formula M-a, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In Formula M-a, “m” may be 0 or 1, and “n” may be 2 or 3. In Formula M-a, when “m” is 0, “n” is 3, and when “m” is 1, “n” is 2.

The compound represented by Formula M-a may be utilized as a red phosphorescence dopant or a green phosphorescence dopant.

The compound represented by Formula M-a may be represented by any one selected from among Compounds M-a1 to M-a25. However, Compounds M-a1 to M-a25 are illustrations, and the compound represented by Formula M-a is not limited to the compounds represented by Compounds M-a1 to M-a25.

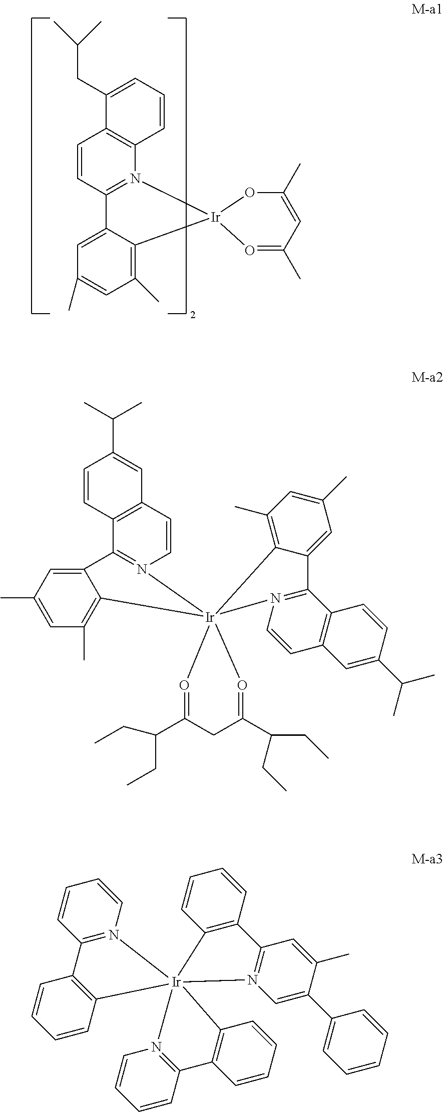

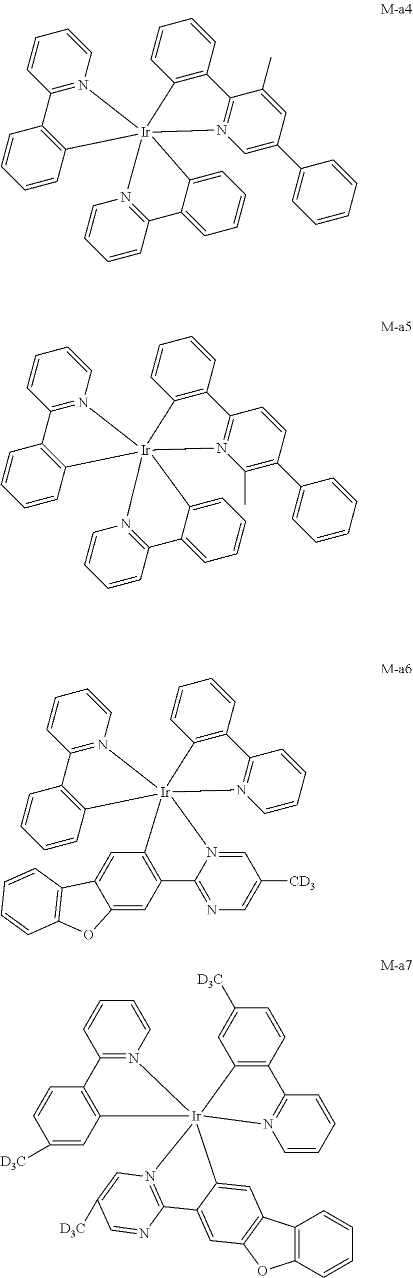

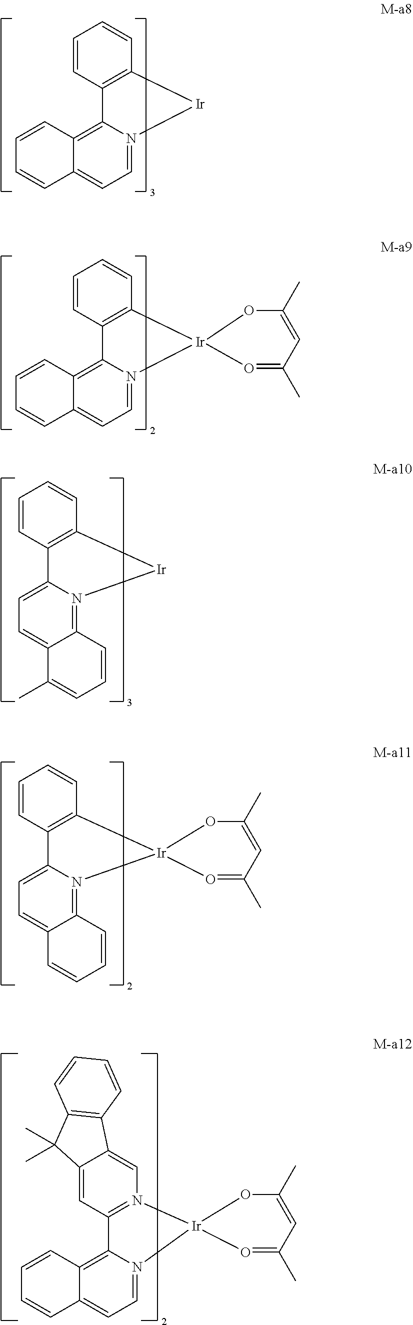

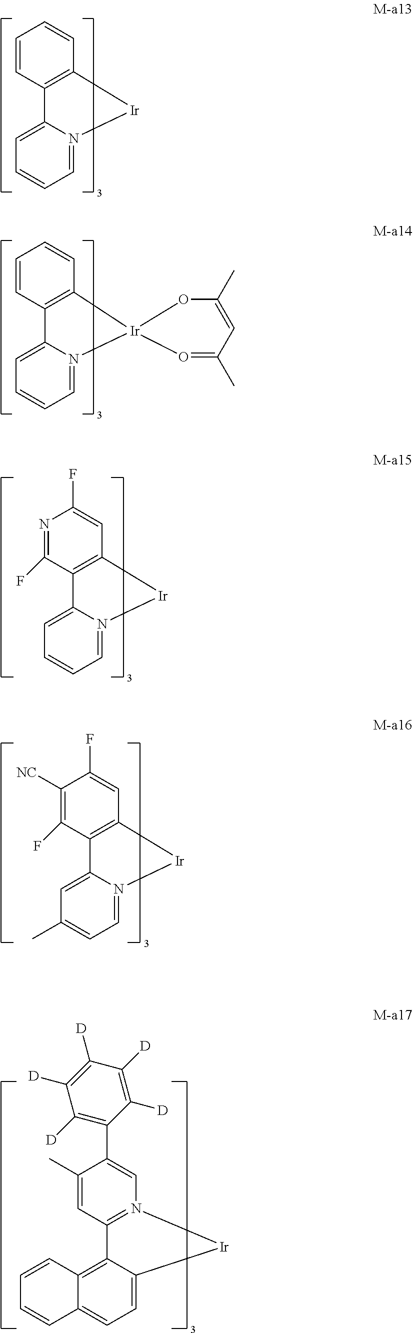

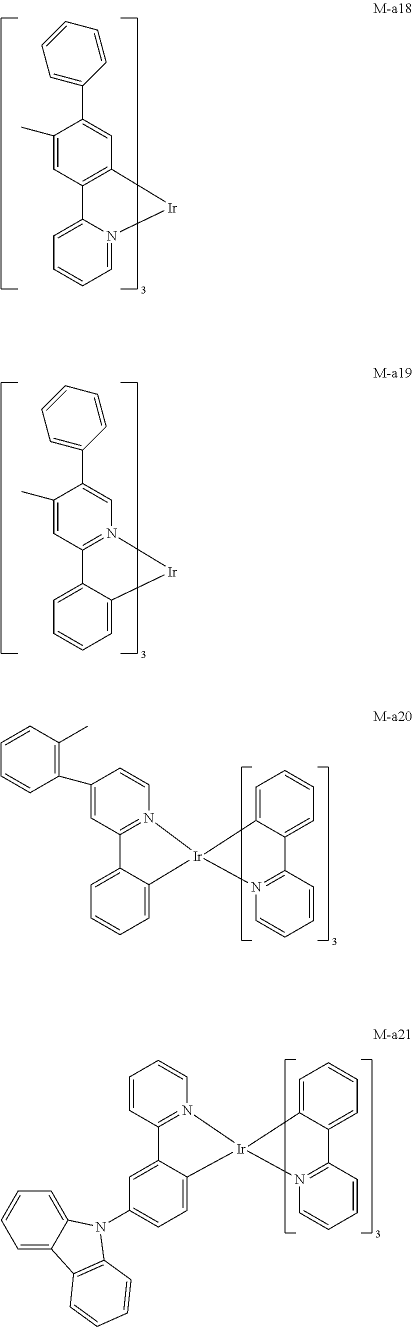

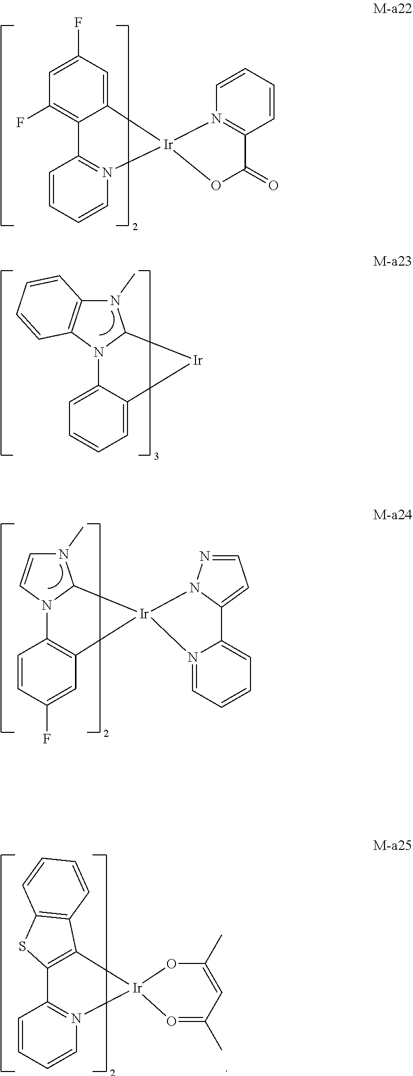

Compound M-a1 and Compound M-a2 may be utilized as red dopant materials, and Compound M-a3 and Compound M-a4 may be utilized as green dopant materials.

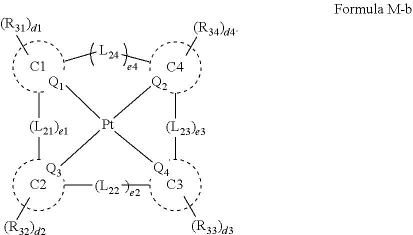

In Formula M-b, Q₁ to Q₄ may each independently be C or N, C1 to C4 may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage,

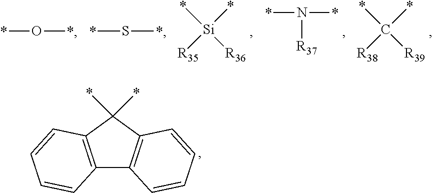

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and d1 to d4 may each independently be an integer of 0 to 4.

The compound represented by Formula M-b may be utilized as a blue phosphorescence dopant or a green phosphorescence dopant.

The compound represented by Formula M-b may be represented by any one selected from among the compounds below. However, the compounds below are illustrations, and the compound represented by Formula M-b is not limited to the compounds represented below.

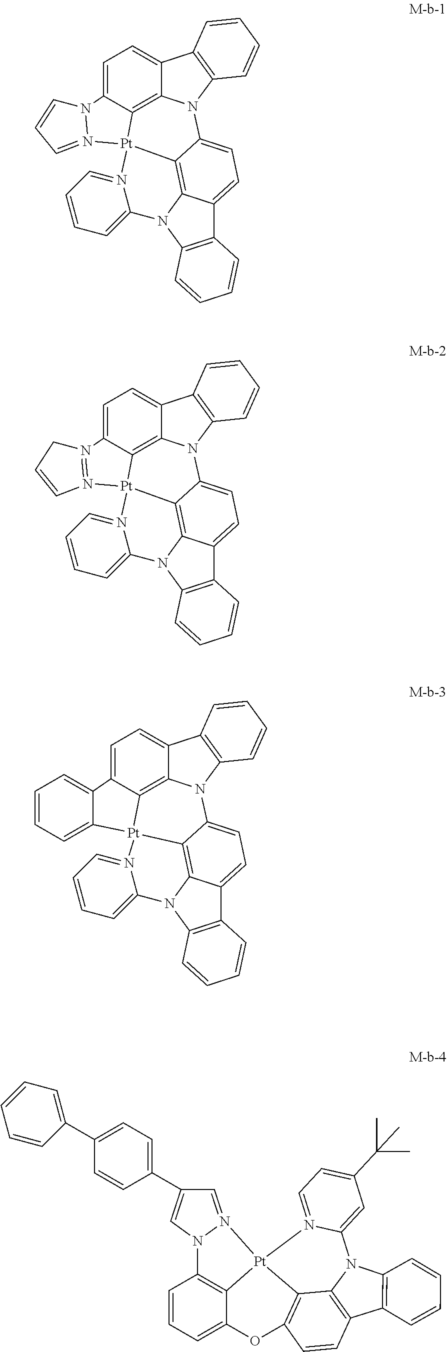

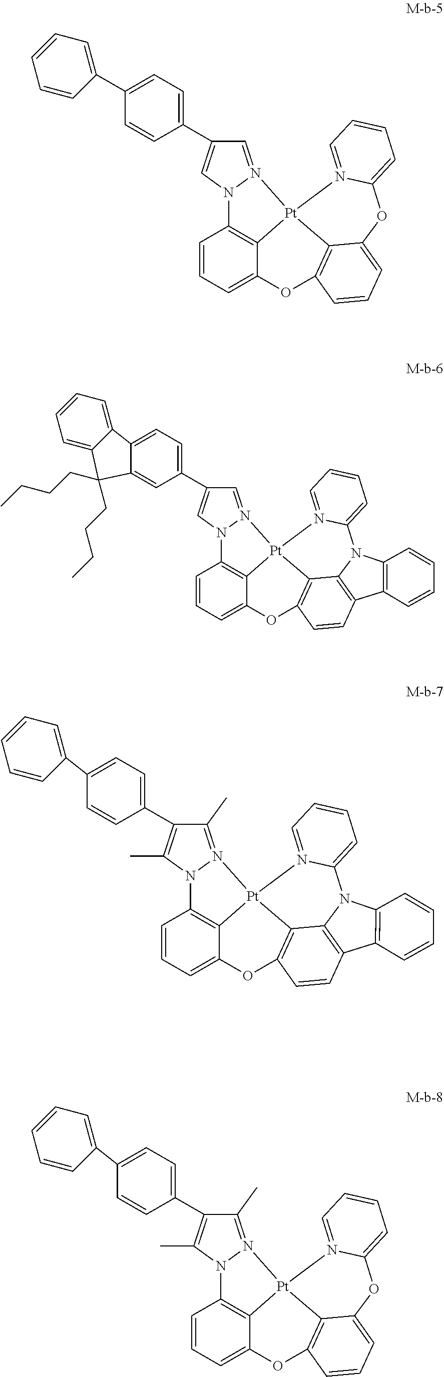

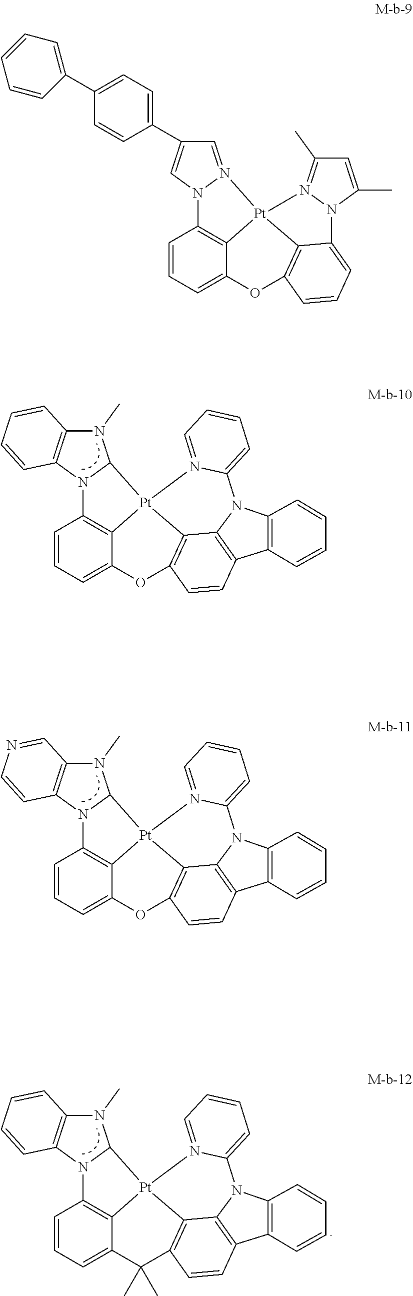

In the compounds above, R, R₃₈, and R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may include any one selected from among Formula F-a to Formula F-c. The compounds represented by Formula F-a to Formula F-c may be utilized as fluorescence dopant materials.

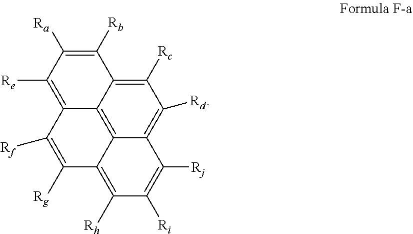

In Formula F-a, two selected from Rₐ to Rⱼ may each independently be substituted with *—NAr₁Ar₂. The remainder not substituted with *—NAr₁Ar₂ among Rₐ to Rⱼ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *—NAr₁Ar₂, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one selected from among Ar₁ and Ar₂ may be a heteroaryl group including O or S as a ring-forming atom.

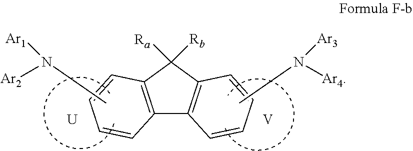

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Ar₁ to Ar₄ may each independently be a substituted or unsubstituted aryl group of 6 to 30 ring-forming carbon atoms, or a substituted or unsubstituted heteroaryl group of 2 to 30 ring-forming carbon atoms.

In Formula F-b, U and V may each independently be 0 or 1. In Formula F-b, U refers to the number of rings combined at position U, and V refers to the number of rings combined at position V. For example, when the number of U or V is 1, the ring designated by U or V forms a fused ring, and when U or V is 0, the ring designated by U or V is not present. For example, when U is 0, and V is 1, or when U is 1, and V is 0, a fused ring having the fluorene core of Formula F-b may be a ring compound with four rings. In some embodiments, when both (e.g., simultaneously) U and V are 0, the fused ring of Formula F-b may be a ring compound with three rings. In some embodiments, when both (e.g., simultaneously) U and V are 1, a fused ring having the fluorene core of Formula F-b may be a ring compound with five rings.

In Formula F-b, when U or V is 1, U and V may each independently be a substituted or unsubstituted hydrocarbon ring of 5 to 30 ring-forming carbon atoms, or a substituted or unsubstituted heterocycle of 2 to 30 ring-forming carbon atoms.

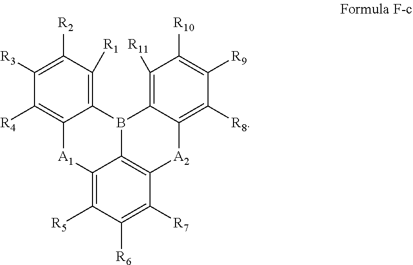

In Formula F-c, A₁ and A2 may each independently be O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In Formula F-c, A₁ and A2 may each independently be combined with the substituents of an adjacent ring to form a fused ring. For example, when A₁ and A2 may be each independently NRₘ, A₁ may be combined with R₄ or R₅ to form a ring. In some embodiments, A2 may be combined with R₇ or R₈ to form a ring.

In an embodiment, the emission layer EML may include, as a suitable dopant material, styryl derivatives (for example, 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), perylene and/or derivatives thereof (for example, 2,5,8,11-tetra-t-butylperylene (TBP)), pyrene and/or derivatives thereof (for example, 1,1-dipyrene, 1,4-dipyrenylbenzene, and/or 1,4-bis(N,N-diphenylamino)pyrene), etc.

The emission layer EML may include a suitable phosphorescence dopant material. For example, the phosphorescence dopant may utilize a metal complex including iridium (Ir), platinum (Pt), osmium (Os), gold (Au), titanium (Ti), zirconium (Zr), hafnium (Hf), europium (Eu), terbium (Tb) or thulium (Tm). For example, iridium(III) bis(4,6-difluorophenylpyridinato-N,C2′)picolinate (Firpic), bis(2,4-difluorophenylpyridinato)-tetrakis(1-pyrazolyl)borate iridium(III) (Fir6), or platinum octaethyl porphyrin (PtOEP) may be utilized as the phosphorescence dopant. However, embodiments of the present disclosure are not limited thereto.

The emission layer EML may include a quantum dot material. The core of the quantum dot may be selected from a Group II-VI compound, a Group III-V compound, a Group IV-VI compound, a Group IV element, a Group IV compound, and combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof.

The Group III-VI compound may include a binary compound (such as In₂S₃, and/or In₂Se₃), a ternary compound (such as InGaS₃ and/or InGaSe₃), or one or more optional combinations thereof.

The Group I-III-VI compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, and a quaternary compound (such as AgInGaS₂ and/or CuInGaS₂).

The Group III-V compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, InAlPSb, and mixtures thereof. In some embodiments, the Group III-V compound may further include a Group II metal. For example, InZnP, etc. may be selected as a Group III-II-V compound.

The Group IV-VI compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The Group IV element may be selected from the group consisting of Si, Ge, and a mixture thereof. The Group IV compound may be a binary compound selected from the group consisting of SiC, SiGe, and a mixture thereof.

In this case, the binary compound, the ternary compound and/or the quaternary compound may each independently be present at a substantially uniform concentration in a particle, or may be present at a partially different (e.g., non-uniform) concentration distribution state in the same particle. In some embodiments, a core/shell structure in which one quantum dot wraps another quantum dot may be possible. The interface of the core and the shell may have a concentration gradient, in which the concentration of an element present in the shell is decreased toward the center.

In some embodiments, the quantum dot may have the above-described core-shell structure including a core including a nanocrystal and a shell wrapping the core. The shell of the quantum dot may play the role of a protection layer for preventing or reducing chemical deformation of the core to maintain semiconductor properties, and/or the role of a charging layer for imparting the quantum dot with electrophoretic properties. The shell may have a single layer or a multilayer. The interface of the core and shell may have concentration gradient of decreasing the concentration of elements present in the shell toward the center. Examples of the shell of the quantum dot may include a metal or non-metal oxide, a semiconductor compound, or combinations thereof.

For example, the metal or non-metal oxide may include a binary compound (such as SiO₂, Al₂O₃, TiO₂, ZnO, MnO, Mn₂O₃, Mn₃O₄, CuO, FeO, Fe₂O₃, Fe₃O₄, CoO, CO₃O₄ and/or NiO), and/or a ternary compound (such as MgAl₂O₄, CoFe₂O₄, NiFe₂O₄ and CoMn₂O₄), but embodiments of the present disclosure are not limited thereto.

In some embodiments, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments of the present disclosure are not limited thereto.

The quantum dot may have a full width of half maximum (FWHM) of emission wavelength spectrum of about 45 nm or less, about 40 nm or less, more, about 30 nm or less. Within this range, color purity or color reproducibility may be improved. In some embodiments, light emitted via such quantum dot is emitted in all directions, and light view angle properties may be improved.

In some embodiments, the shape of the quantum dot may be any generally utilized shape in the art, without specific limitation. For example, the shape may be a spherical, pyramidal, multi-arm, or cubic nanoparticle, nanotube, nanowire, nanofiber, nanoplate particle, etc.

The quantum dot may control the color of light emitted, according to the particle size, and accordingly, the quantum dot may have one or more suitable emission colors (such as blue, red and green).

In the luminescence device ED of an embodiment, as shown in FIG. 3 to FIG. 6, the electron transport region ETR is provided on the emission layer EML. The electron transport region ETR may include at least one of a hole blocking layer HBL, an electron transport layer ETL or an electron injection layer EIL. However, embodiments of the present disclosure are not limited thereto.

The electron transport region ETR may have a single layer formed utilizing a single material, a single layer formed utilizing multiple different materials, or a multilayer structure having multiple layers formed utilizing multiple different materials.

For example, the electron transport region ETR may have a single layer structure of an electron injection layer EIL or an electron transport layer ETL, or a single layer structure formed utilizing an electron injection material and an electron transport material. Further, the electron transport region ETR may have a single layer structure formed utilizing multiple different materials, or a structure stacked from the emission layer EML of electron transport layer ETL/electron injection layer EIL, or hole blocking layer HBL/electron transport layer ETL/electron injection layer EIL, without limitation. The thickness of the electron transport region ETR may be, for example, about 1,000 Å to about 1,500 Å.

The electron transport region ETR may be formed utilizing one or more suitable methods (such as a vacuum deposition method, a spin coating method, a cast method, a Langmuir-Blodgett (LB) method, an inkjet printing method, a laser printing method, and/or a laser induced thermal imaging (LITI) method).

The electron transport region ETR may include a compound represented by Formula ET-1.

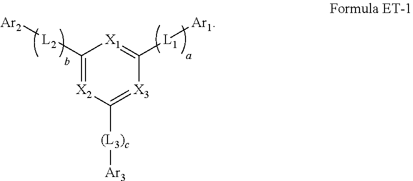

In Formula ET-1, at least one selected from among X₁ to X₃ may be N, and the remainder may be CRₐ. Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, “a” to “c” may each independently be an integer of 0 to 10. In Formula ET-1, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” to “c” are integers of 2 or more, L₁ to L₃ may each independently be a substituted or unsubstituted arylene group of 6 to 30 ring-forming carbon atoms, or a substituted or unsubstituted heteroarylene group of 2 to 30 ring-forming carbon atoms.

When the electron transport region ETR includes an electron transport layer ETL, the electron transport region ETR may include an anthracene-based compound. However, embodiments of the present disclosure are not limited thereto, and the electron transport region ETR may include, for example, tris(8-hydroxyquinolinato)aluminum (Alq3), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), or one or more mixtures thereof, without limitation.

The electron transport region ETR may include at least one selected from among Compounds ET1 to ET36.

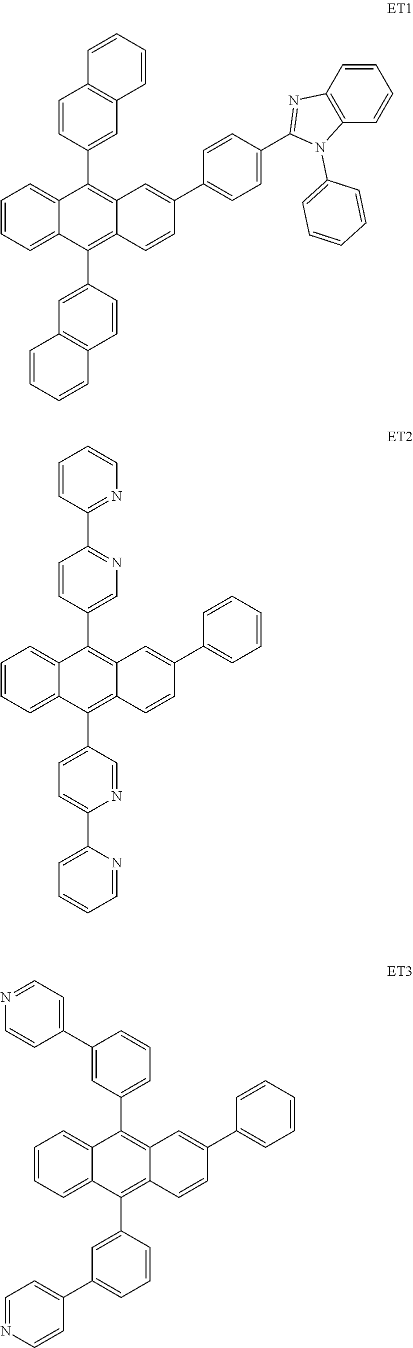 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

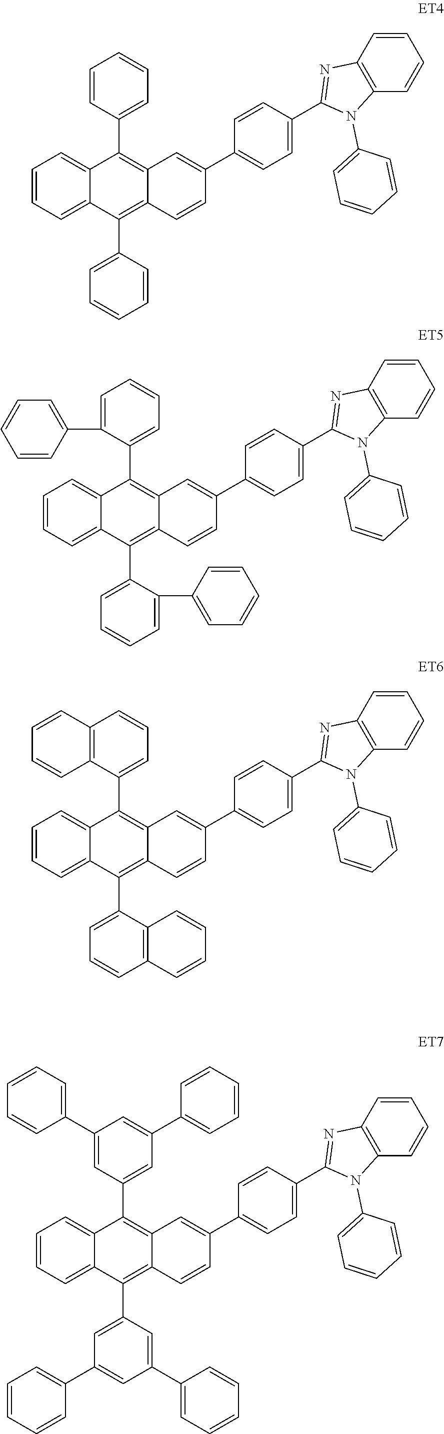 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

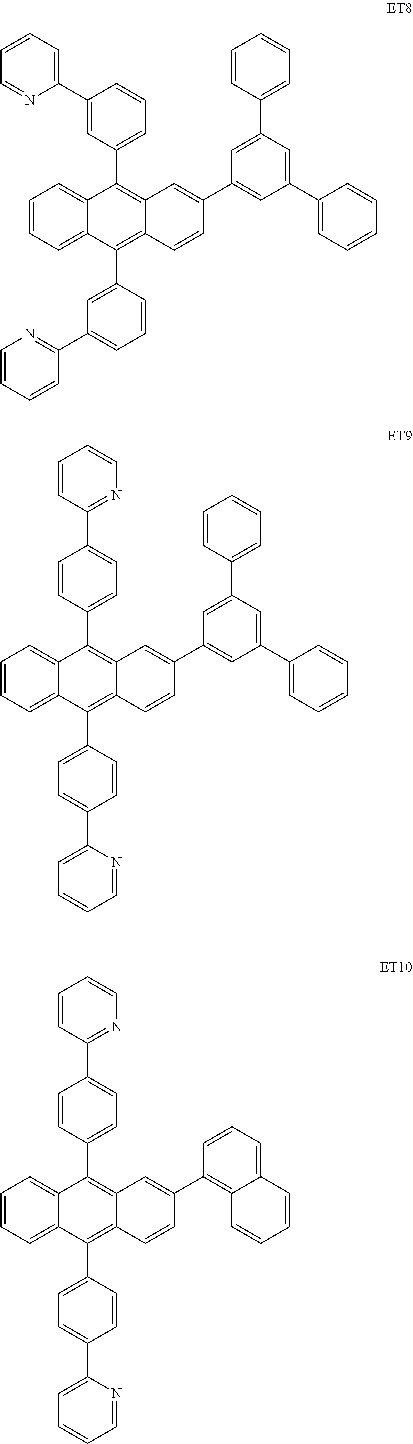 [SMILES: c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1 ; c1ccc(-c2ccc(-c3c4ccccc4c(-c4ccc(-c5ccccn5)cc4)c4cc(-c5cccc6ccccc56)ccc34)cc2)nc1]

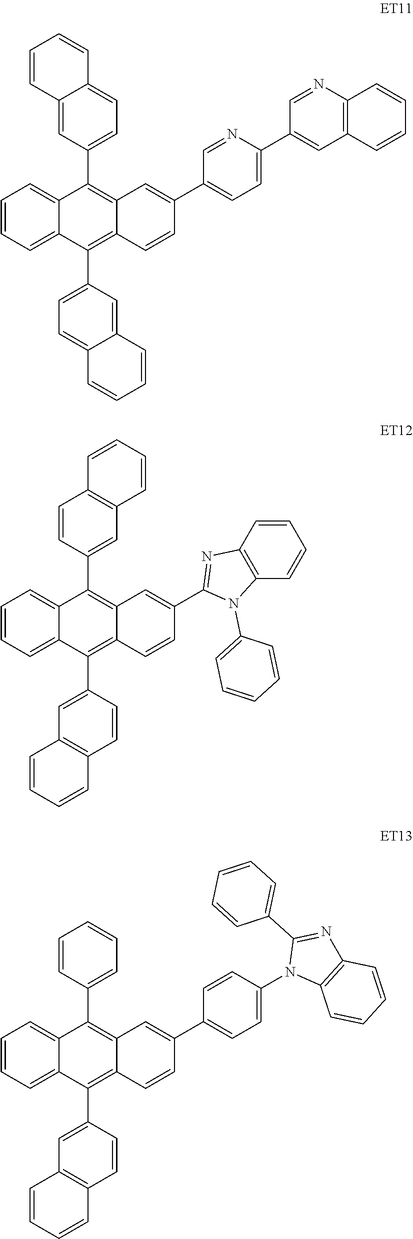

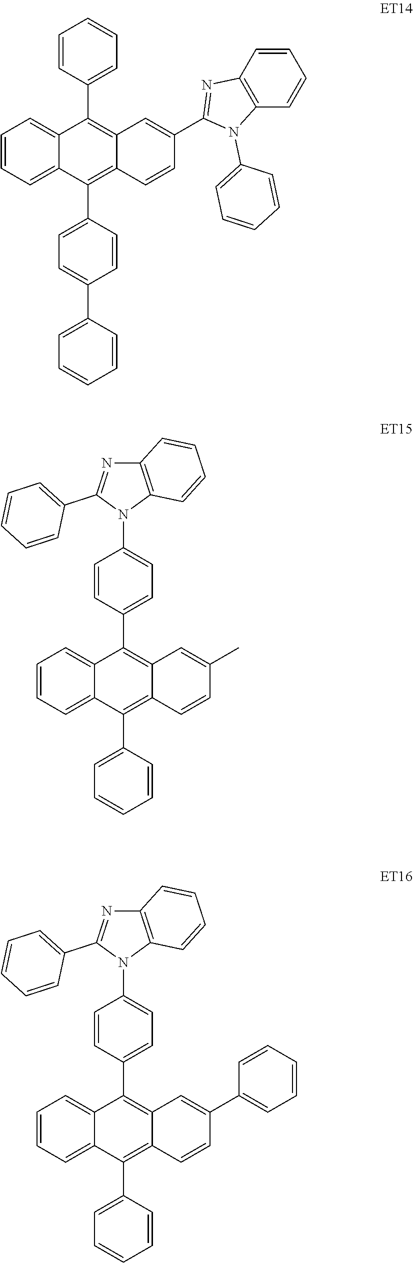 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

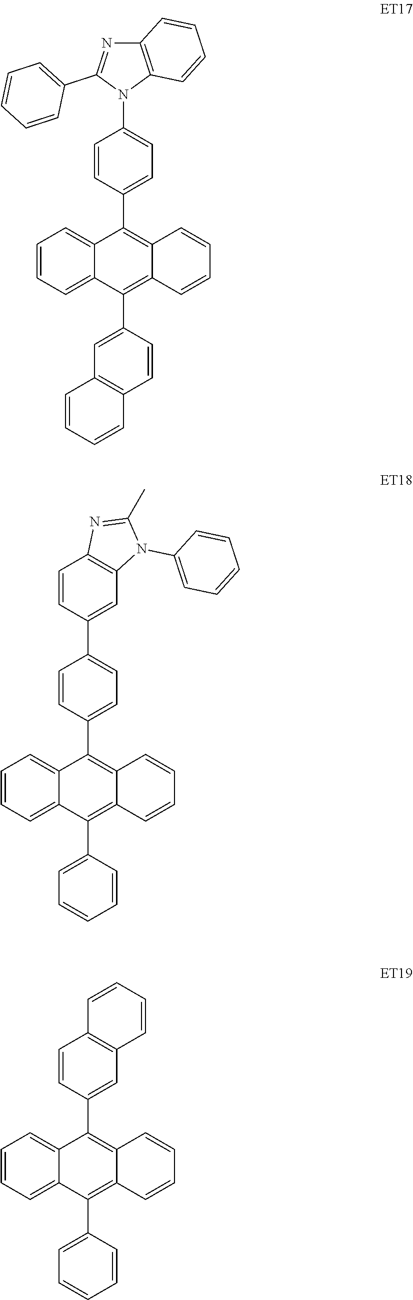

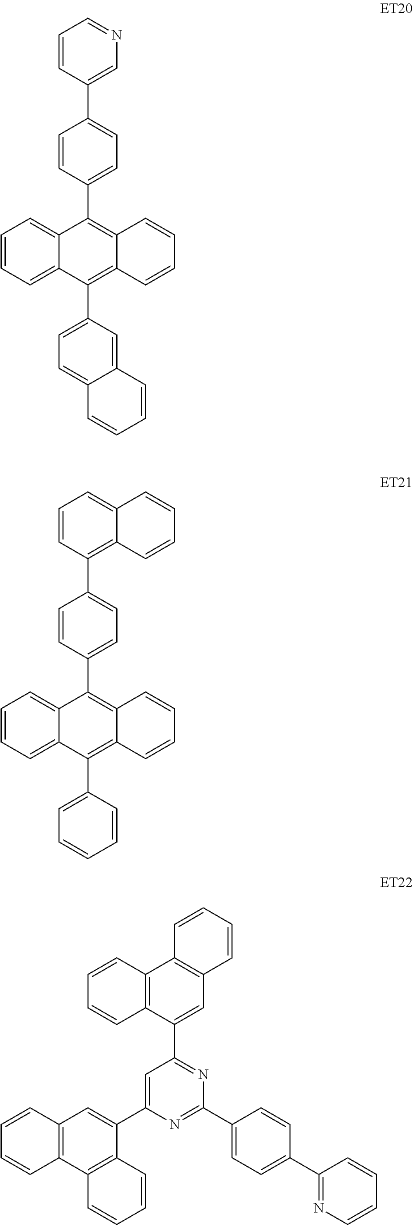

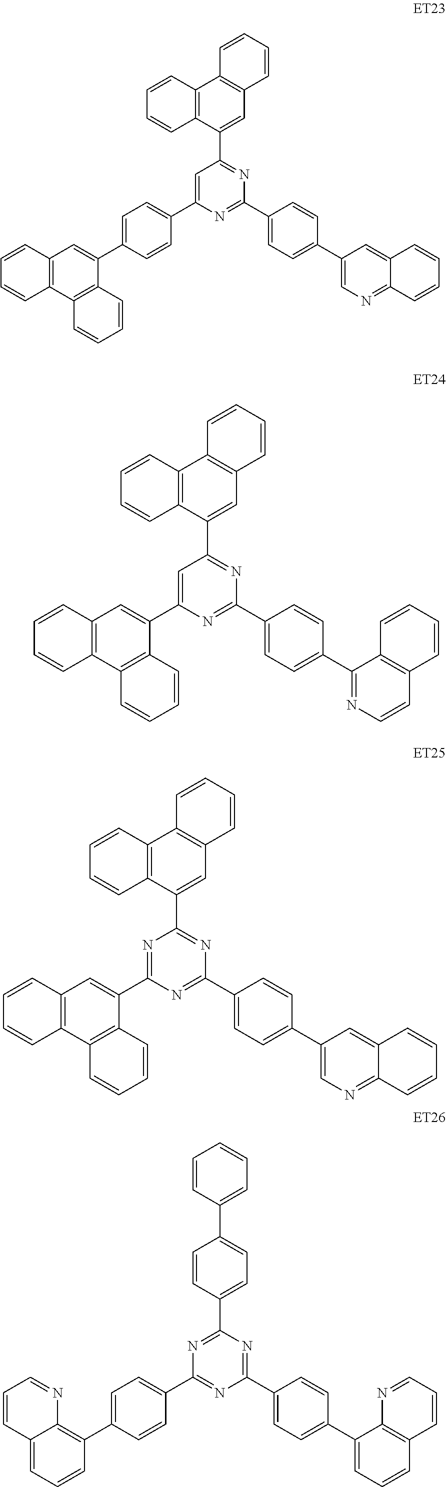 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1]

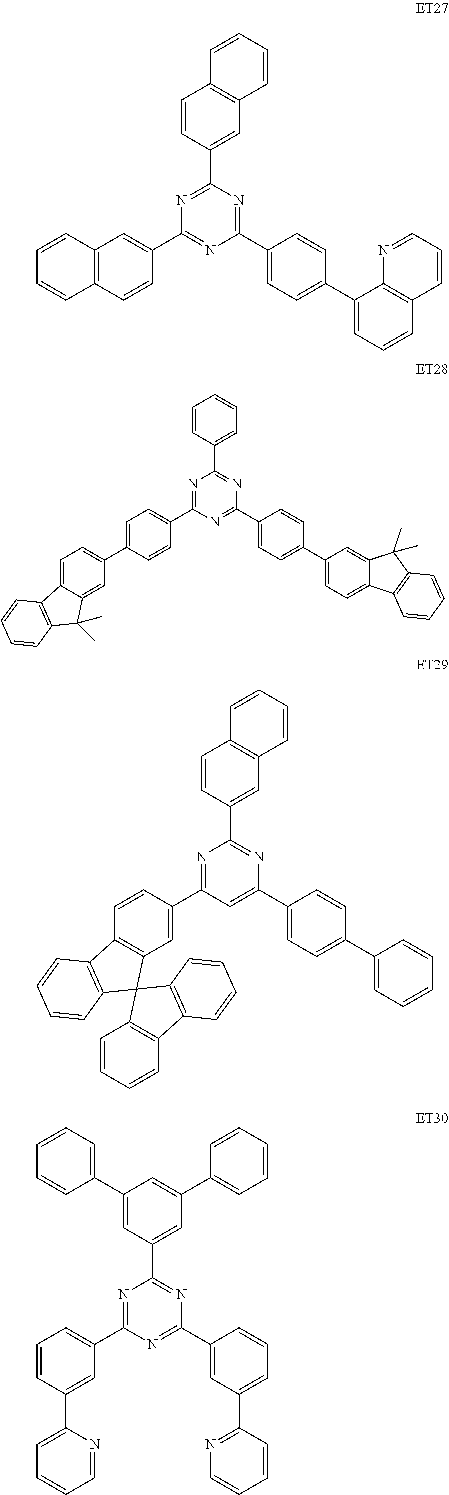 [SMILES: c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

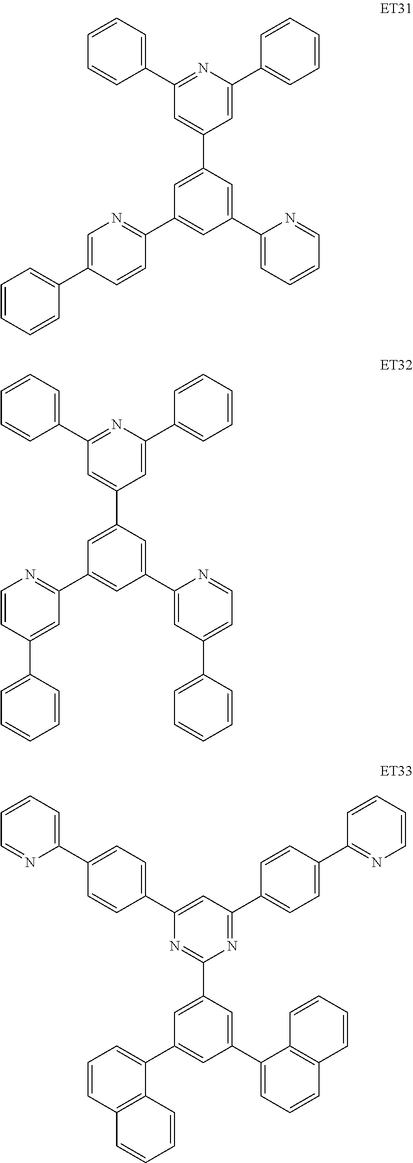

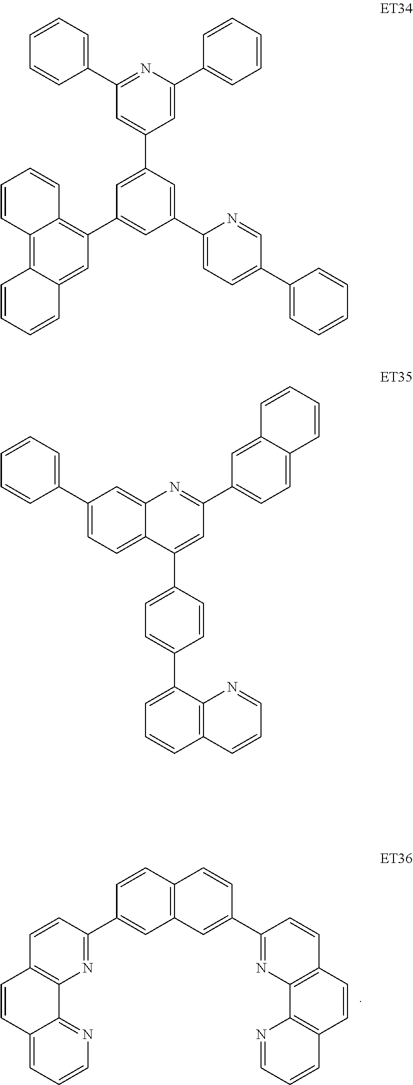 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide (such as LiF, NaCl, CsF, RbCl, RbI, CuI and/or KI), a lanthanide metal (such as Yb), or a co-depositing material of the metal halide and the lanthanide metal. For example, the electron transport region ETR may include KI:Yb, RbI:Yb, etc., as the co-depositing material. In some embodiments, the electron transport region ETR may utilize a metal oxide (such as Li₂O and/or BaO), or 8-hydroxy-lithium quinolate (LiQ). However, embodiments of the present disclosure are not limited thereto. The electron transport region ETR also may be formed utilizing a mixture of an electron transport material and an insulating organo metal salt. The organo metal salt may be a material having an energy band gap of about 4 eV or more. For example, the organo metal salt may include, for example, metal acetates, metal benzoates, metal acetoacetates, metal acetylacetonates, and/or metal stearates. However, embodiments of the present disclosure are not limited thereto.

The electron transport region ETR may include the compounds of the electron transport region in at least one selected from among an electron injection layer EIL, an electron transport layer ETL, and a hole blocking layer HBL.

When the electron transport region ETR includes the electron transport layer ETL, the thickness of the electron transport layer ETL may be about 100 Å to about 1,000 Å, for example, about 150 Å to about 500 Å. When the thickness of the electron transport layer ETL satisfies the above-described range, satisfactory electron transport properties may be obtained without a substantial increase in driving voltage. When the electron transport region ETR includes the electron injection layer EIL, the thickness of the electron injection layer EIL may be about 1 Å to about 100 Å, and about 3 Å to about 90 Å. When the thickness of the electron injection layer EIL satisfies the above described range, satisfactory electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but embodiments of the present disclosure are not limited thereto. For example, when the first electrode EL1 is an anode, the second cathode EL2 may be a cathode, and when the first electrode EL1 is a cathode, the second electrode EL2 may be an anode.

The second electrode EL2 may be a transmissive electrode, a transflective electrode or a reflective electrode. When the second electrode EL2 is a transmissive electrode, the second electrode EL2 may include a transparent metal oxide, for example, ITO, IZO, ZnO, ITZO, etc.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, one or more compounds thereof, or one or more mixtures thereof (for example, AgMg, AgYb, or MgAg). In some embodiments, the second electrode EL2 may have a multilayered structure including a reflective layer or a transflective layer formed utilizing the above-described materials and a transparent conductive layer formed utilizing ITO, IZO, ZnO, ITZO, etc. For example, the second electrode EL2 may include the aforementioned metal materials, combinations of two or more metal materials selected from the aforementioned metal materials, or oxides of the aforementioned metal materials.

In some embodiments, the second electrode EL2 may be connected with an auxiliary electrode. When the second electrode EL2 is connected with the auxiliary electrode, the resistance of the second electrode EL2 may decrease.

In some embodiments, a capping layer CPL may be further disposed on the second electrode EL2 in the luminescence device ED of an embodiment. The capping layer CPL may include a multilayer or a single layer.

In an embodiment, the capping layer CPL may be an organic layer or an inorganic layer. For example, when the capping layer CPL includes an inorganic material, the inorganic material may include an alkali metal compound (such as LiF), an alkaline earth metal compound (such as MgF₂, SiON, SiNₓ, SiO_{y}), etc.

For example, when the capping layer CPL includes an organic material, the organic material may include α-NPD, NPB, TPD, m-MTDATA, Alq3, CuPc, N4,N4,N4′,N4′-tetra(biphenyl-4-yl) biphenyl-4,4′-diamine (TPD15), 4,4′,4″-tris(carbazol sol-9-yl) triphenylamine (TCTA), etc., or includes an epoxy resin, or acrylate (such as methacrylate). In some embodiments, a capping layer CPL may include at least one selected from among Compounds P1 to P5, but embodiments of the present disclosure are not limited thereto.

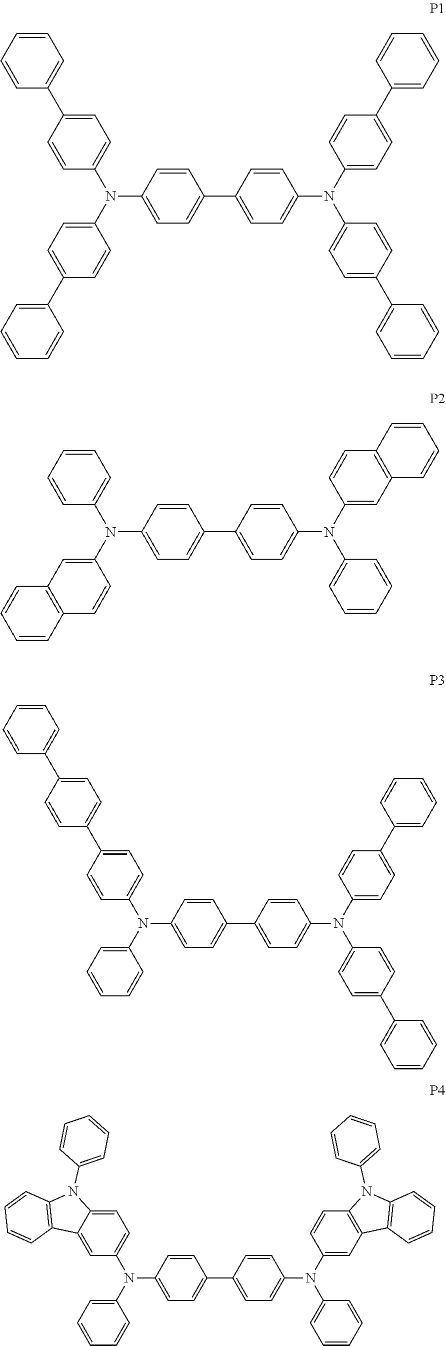 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

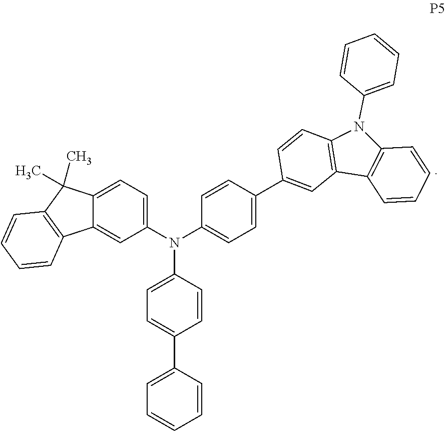 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL with respect to light in a wavelength range of about 550 nm to about 660 nm may be about 1.6 or more.

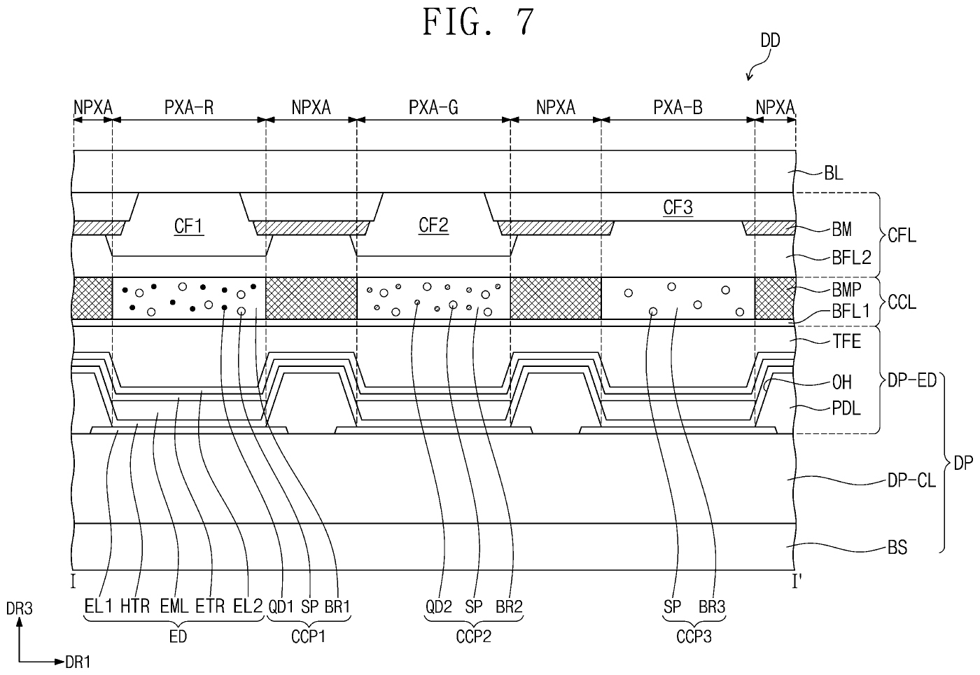

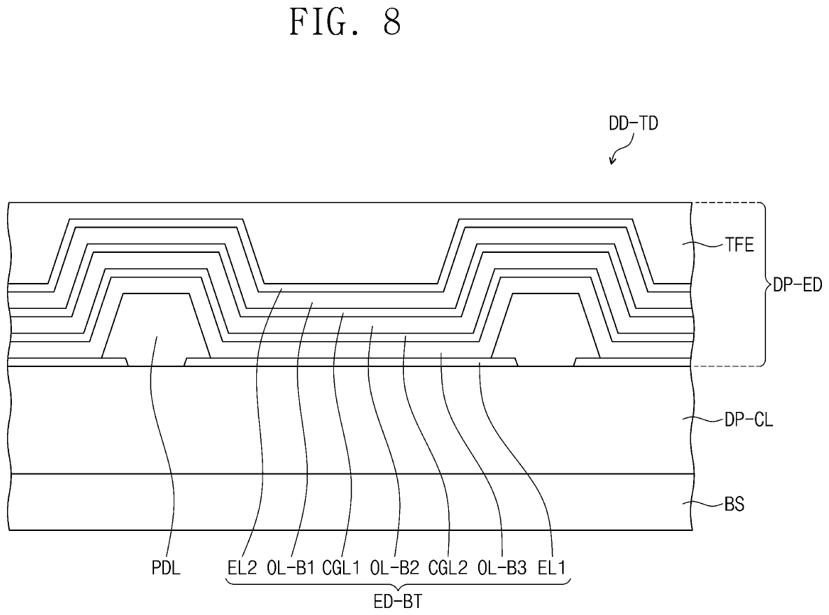

FIG. 7 and FIG. 8 are cross-sectional views on display apparatuses according to embodiments, respectively. In the explanation on the display apparatuses of embodiments referring to FIG. 7 and FIG. 8, the overlapping parts with the explanation on FIG. 1 to FIG. 6 will not be explained again, and the different features will be explained chiefly.

Referring to FIG. 7, the display apparatus DD according to an embodiment may include a display panel DP including a display device layer DP-ED, a light controlling layer CCL disposed on the display panel DP and a color filter layer CFL.

In an embodiment shown in FIG. 7, the display panel DP includes a base layer BS, a circuit layer DP-CL provided on the base layer BS and a display device layer DP-ED, and the display device layer DP-ED may include a luminescence device ED.

The luminescence device ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. The structures of the luminescence devices of FIG. 3 to FIG. 6 may be applied to the structure of the luminescence device ED shown in FIG. 7.

Referring to FIG. 7, the emission layer EML may be disposed in an opening part OH defined in a pixel definition layer PDL. For example, the emission layer EML divided by the pixel definition layer PDL and correspondingly provided to each of luminous areas PXA-R, PXA-G and PXA-B may be to emit light in the same wavelength region. In the display apparatus DD of an embodiment, the emission layer EML may be to emit blue light. In some embodiments, different from the drawings, in an embodiment, the emission layer EML may be provided as a common layer for all luminous areas PXA-R, PXA-G and PXA-B.

The light controlling layer CCL may be disposed on the display panel DP. The light controlling layer CCL may include a light converter. The light converter may be a quantum dot or a phosphor. The light converter may transform the wavelength of light provided and then emit (e.g., emit a different color light). For example, the light controlling layer CCL may be a layer including a quantum dot or a layer including a phosphor.

The light controlling layer CCL may include multiple light controlling parts CCP1, CCP2 and CCP3. The light controlling parts CCP1, CCP2 and CCP3 may be separated from one another.

Referring to FIG. 7, a partition pattern BMP may be disposed between the separated light controlling parts CCP1, CCP2 and CCP3, but embodiments of the present disclosure are not limited thereto. In FIG. 7, the partition pattern BMP is shown not to be overlapped with the light controlling parts CCP1, CCP2 and CCP3, but at least a portion of the edge of the light controlling parts CCP1, CCP2 and CCP3 may be overlapped with the partition pattern BMP.

The light controlling layer CCL may include a first light controlling part CCP1 including a first quantum dot QD1 converting first color light provided from the luminescence device ED into second color light, a second light controlling part CCP2 including a second quantum dot QD2 converting first color light into third color light, and a third light controlling part CCP3 transmitting first color light.

In an embodiment, the first light controlling part CCP1 may provide red light (which is the second color light), and the second light controlling part CCP2 may provide green light (which is the third color light). The third color controlling part CCP3 may transmit and provide blue light (which is the first color light provided from the luminescence device ED). For example, the first quantum dot QD1 may be a red quantum dot, and the second quantum dot QD2 may be a green quantum dot. For the quantum dots QD1 and QD2, the same description as above may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but include the scatterer SP.

The scatterer SP may be an inorganic particle. For example, the scatterer SP may include at least one selected from among TiO₂, ZnO, Al₂O₃, SiO₂, and hollow silica. The scatterer SP may include at least one selected from among TiO₂, ZnO, Al₂O₃, SiO₂, and hollow silica, or may be a mixture of two or more materials selected from among TiO₂, ZnO, Al₂O₃, SiO₂, and hollow silica.

The light controlling layer CCL may include a barrier layer BFL1. The barrier layer BFL1 may play the role of blocking or reducing the penetration of moisture and/or oxygen (hereinafter, will be referred to as “humidity/oxygen”). The barrier layer BFL1 may be disposed on the light controlling parts CCP1, CCP2 and/or CCP3 to block or reduce the exposure of the light controlling parts CCP1, CCP2 and CCP3 to humidity/oxygen. In some embodiments, the barrier layer BFL1 may cover the light controlling parts CCP1, CCP2 and CCP3. In some embodiments, the barrier layer BFL2 may be provided between a color filter layer CFL and one or more of the light controlling parts CCP1, CCP2 and CCP3.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride and/or a metal thin film for securing light transmittance. In some embodiments, the barrier layers BFL1 and BFL2 may further include an organic layer. The barrier layers BFL1 and BFL2 may be composed of a single layer of multiple layers.

In the display apparatus DD of an embodiment, the color filter layer CFL may be disposed on the light controlling layer CCL. For example, the color filter layer CFL may be disposed directly on the light controlling layer CCL. In this case, the barrier layer BFL2 may not be provided.

The color filter layer CFL may include a light blocking part BM and filters CF1, CF2 and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment or dye. The first filter CF1 may include a red pigment and/or dye, the second filter CF2 may include a green pigment and/or dye, and the third filter CF3 may include a blue pigment and/or dye. In some embodiments, embodiments of the present disclosure are not limited thereto, and the third filter CF3 may not include the pigment or dye. The third filter CF3 may include a polymer photosensitive resin and not include a pigment or dye. The third filter CF3 may be transparent. The third filter CF3 may be formed utilizing a transparent photosensitive resin.

In some embodiments, the first filter CF1 and/or the second filter CF2 may be yellow filters. The first filter CF1 and the second filter CF2 may be provided in one body without distinction.

The light blocking part BM may be a black matrix. The light blocking part BM may be formed by including an organic light blocking material or an inorganic light blocking material including a black pigment or black dye. The light blocking part BM may prevent or reduce light leakage phenomenon and divide the (e.g., act as) boundaries among adjacent filters CF1, CF2 and CF3. In some embodiments, the light blocking part BM may be formed as a blue filter.

Each of the first to third filters CF1, CF2 and CF3 may be disposed to respectively correspond to a red luminous area PXA-R, green luminous area PXA-G, and blue luminous area PXA-B.

On the color filter layer CFL, a base substrate BL may be disposed. The base substrate BL may provide a base surface on which the color filter layer CFL, the light controlling layer CCL, etc. are disposed. The base substrate BL may be a glass substrate, a metal substrate, a plastic substrate, etc. However, embodiments of the present disclosure are not limited thereto, and the base substrate BL may be an inorganic layer, an organic layer or a composite material layer. In some embodiments, the base substrate BL may not be provided.

FIG. 8 is a cross-sectional view showing a portion of the display apparatus according to an embodiment. In FIG. 8, the cross-sectional view of a portion corresponding to the display panel DP in FIG. 7 is shown. In a display apparatus DD-TD of an embodiment, the luminescence device ED-BT may include multiple light emitting structures OL-B1, OL-B2 and OL-B3. The luminescence device ED-BT may include a first electrode EL1 and second electrode EL2 oppositely disposed, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include an emission layer EML (FIG. 7), and a hole transport region HTR and an electron transport region ETR disposed with the emission layer EML (FIG. 7) therebetween.

For example, the luminescence device ED-BT included in the display apparatus DD-TD of an embodiment may be a luminescence device of a tandem structure including multiple emission layers.

In an embodiment shown in FIG. 8, light emitted from the light emitting structures OL-B1, OL-B2 and OL-B3 may be all blue light. However, embodiments of the present disclosure are not limited thereto, and the wavelength regions of light emitted from the light emitting structures OL-B1, OL-B2 and OL-B3 may be different from each other. For example, the luminescence device ED-BT including the multiple light emitting structures OL-B1, OL-B2 and OL-B3 emitting light in different wavelength regions may be to emit white light.

A charge generating layer CGL1 and CGL2 may be disposed between neighboring light emitting structures OL-B1, OL-B2 and OL-B3. The charge generating layer CGL1 and CGL2 may include a p-type charge generating layer and/or an n-type charge generating layer.

Hereinafter, the present disclosure will be explained referring to embodiments and comparative embodiments. The embodiments are only illustrations to assist the understanding of the present disclosure, and the scope of the present disclosure is not limited thereto.

### SYNTHETIC EXAMPLES

The amine compound according to embodiments of the present disclosure may be synthesized, for example, as follows. However, the synthetic method of the amine compound according to embodiments of the present disclosure is not limited to the embodiments.

### 1. Synthesis of Compound A2

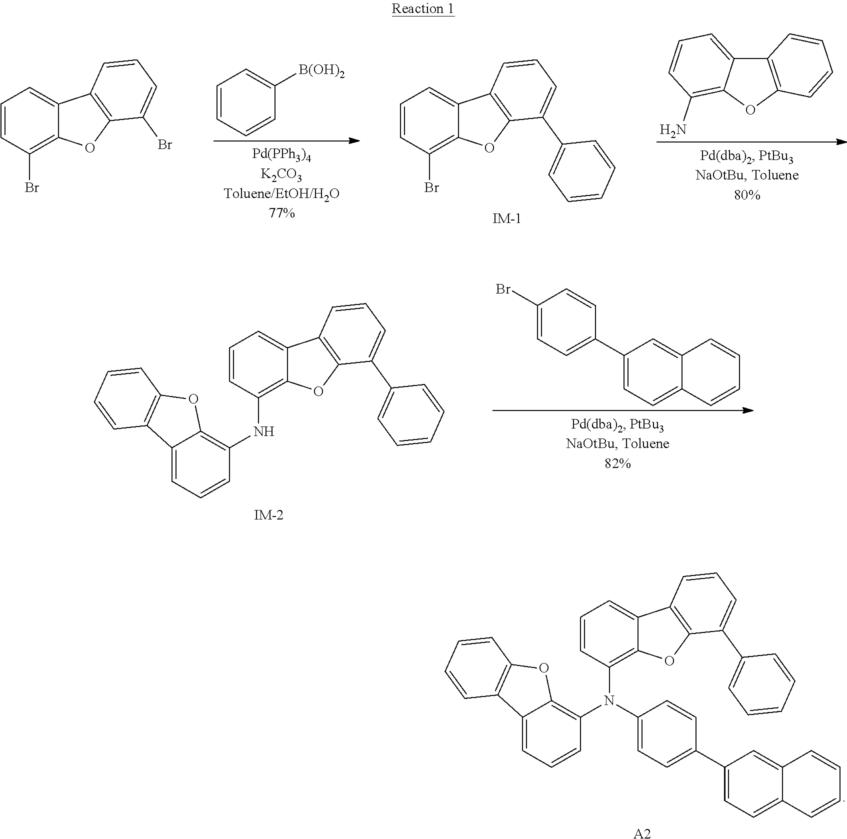

### Synthesis of Intermediate IM-1

Under an Ar atmosphere, to a 1000 mL, three-neck flask, 4,6-dibromodibenzofuran (25.00 g, 76.7 mmol), phenylboronic acid (10.29 g, 1.1 equiv, 84.4 mmol), K₂CO₃ (31.80 g, 3.0 equiv, 230.1 mmol), Pd(PPh₃)₄ (4.43 g, 0.05 eq, 3.8 mmol), and 540 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-1 (19.08 g, yield 77%).

Through FAB-MS measurement, mass number m/z=323 was observed as an ion peak, and Intermediate IM-1 was identified.

### Synthesis of Intermediate IM-2

Under an Ar atmosphere, to a 500 mL, three-neck flask, IM-1 (15.00 g, 46.4 mmol), Pd(dba)₂ (0.80 g, 0.03 equiv, 1.4 mmol), NaOtBu (4.46 g, 1.0 equiv, 46.4 mmol), toluene (232 mL), 4-aminodibenzofuran (9.35 g, 1.1 equiv, 51.1 mmol) and tBu₃P (0.94 g, 0.1 equiv, 4.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-2 (15.79 g, yield 80%).

Through FAB-MS measurement, mass number m/z=425 was observed as an ion peak, and Intermediate IM-2 was identified.

### Synthesis of Compound A2

Under an Ar atmosphere, to a 300 mL, three-neck flask, IM-2 (10.00 g, 23.5 mmol), Pd(dba)₂ (0.41 g, 0.03 equiv, 0.7 mmol), NaOtBu (4.52 g, 2.0 equiv, 47.0 mmol), toluene (118 mL), 2-(4-bromophenyl)naphthalene (7.32 g, 1.1 equiv, 25.9 mmol) and tBu₃P (0.48 g, 0.1 equiv, 2.4 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound A2 of a solid (12.10 g, yield 82%).

Through FAB-MS measurement, mass number m/z=627 was observed as an ion peak, and Compound A2 was identified.

### 2. Synthesis of Compound A53

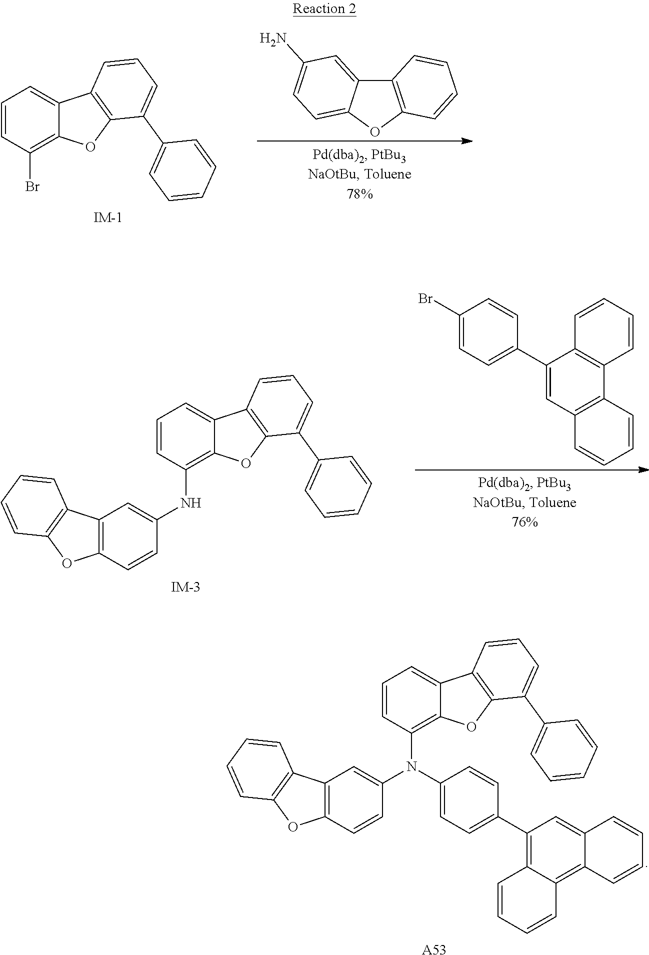

### Synthesis of Intermediate IM-3

Under an Ar atmosphere, to a 500 mL, three-neck flask, IM-1 (15.00 g, 46.4 mmol), Pd(dba)₂ (0.80 g, 0.03 equiv, 1.4 mmol), NaOtBu (4.46 g, 1.0 equiv, 46.4 mmol), toluene (232 mL), 2-aminodibenzofuran (9.35 g, 1.1 equiv, 51.1 mmol) and tBu₃P (0.94 g, 0.1 equiv, 4.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-3 (15.40 g, yield 78%).

Through FAB-MS measurement, mass number m/z=425 was observed as an ion peak, and Intermediate IM-3 was identified.

### Synthesis of Compound A53

Under an Ar atmosphere, to a 300 mL, three-neck flask, IM-3 (10.00 g, 23.5 mmol), Pd(dba)₂ (0.41 g, 0.03 equiv, 0.7 mmol), NaOtBu (4.52 g, 2.0 equiv, 47.0 mmol), toluene (118 mL), 9-(4-bromophenyl)phenanthrene (8.61 g, 1.1 equiv, 25.9 mmol) and tBu₃P (0.48 g, 0.1 equiv, 2.4 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound A53 of a solid (12.43 g, yield 76%).

Through FAB-MS measurement, mass number m/z=677 was observed as an ion peak, and Compound A53 was identified.

### 3. Synthesis of Compound B31

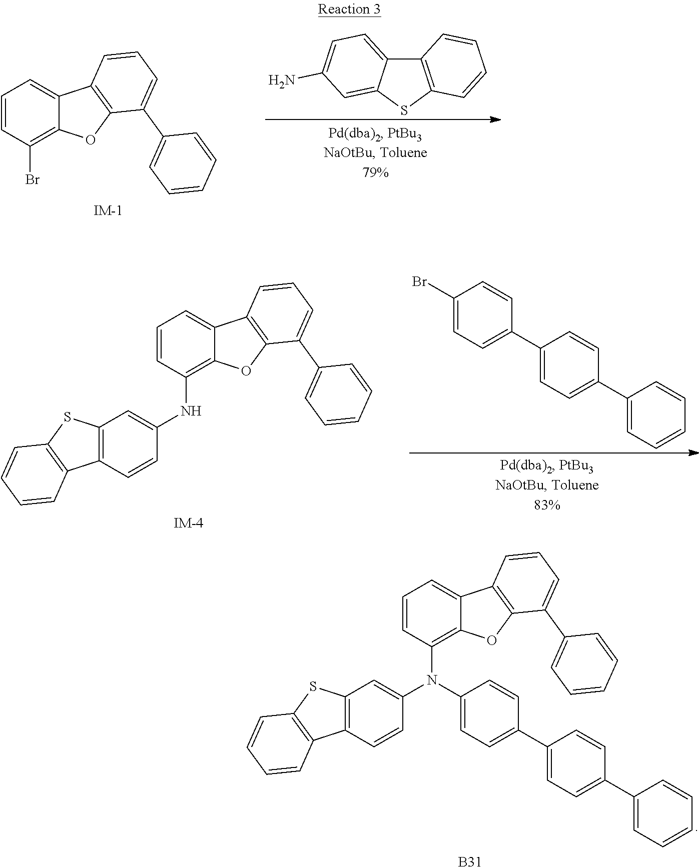

### Synthesis of Intermediate IM-4

Under an Ar atmosphere, to a 500 mL, three-neck flask, IM-1 (15.00 g, 46.4 mmol), Pd(dba)₂ (0.80 g, 0.03 equiv, 1.4 mmol), NaOtBu (4.46 g, 1.0 equiv, 46.4 mmol), toluene (232 mL), 3-aminodibenzothiophene (10.17 g, 1.1 equiv, 51.1 mmol) and tBu₃P (0.94 g, 0.1 equiv, 4.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-4 (16.19 g, yield 79%).

Through FAB-MS measurement, mass number m/z=441 was observed as an ion peak, and Intermediate IM-4 was identified.

### Synthesis of Compound B31

Under an Ar atmosphere, to a 300 mL, three-neck flask, IM-4 (10.00 g, 22.6 mmol), Pd(dba)₂ (0.39 g, 0.03 equiv, 0.7 mmol), NaOtBu (4.35 g, 2.0 equiv, 45.3 mmol), toluene (113 mL), 4-bromo-1,1′:4′,1″-terphenyl (7.70 g, 1.1 equiv, 24.9 mmol) and tBu₃P (0.46 g, 0.1 equiv, 2.3 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound B31 of a solid (12.59 g, yield 83%).

Through FAB-MS measurement, mass number m/z=669 was observed as an ion peak, and Compound B31 was identified.

### 4. Synthesis of Compound B88

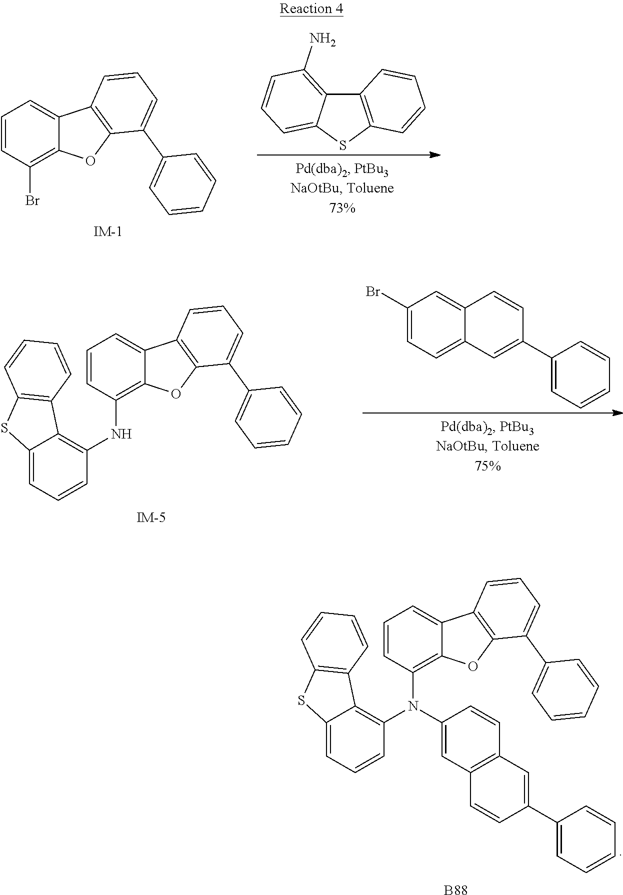

### Synthesis of Intermediate IM-5

Under an Ar atmosphere, to a 500 mL, three-neck flask, IM-1 (15.00 g, 46.4 mmol), Pd(dba)₂ (0.80 g, 0.03 equiv, 1.4 mmol), NaOtBu (4.46 g, 1.0 equiv, 46.4 mmol), toluene (232 mL), 1-aminodibenzothiophene (10.17 g, 1.1 equiv, 51.1 mmol) and tBu₃P (0.94 g, 0.1 equiv, 4.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-5 (14.96 g, yield 73%).

Through FAB-MS measurement, mass number m/z=441 was observed as an ion peak, and Intermediate IM-5 was identified.

### Synthesis of Compound B88

Under an Ar atmosphere, to a 300 mL, three-neck flask, IM-5 (10.00 g, 22.6 mmol), Pd(dba)₂ (0.39 g, 0.03 equiv, 0.7 mmol), NaOtBu (4.35 g, 2.0 equiv, 45.3 mmol), toluene (113 mL), 2-bromo-6-phenylnaphthalene (7.05 g, 1.1 equiv, 24.9 mmol) and tBu₃P (0.46 g, 0.1 equiv, 2.3 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound B88 of a solid (10.94 g, yield 75%).

Through FAB-MS measurement, mass number m/z=643 was observed as an ion peak, and Compound B88 was identified.

### 5. Synthesis of Compound C19

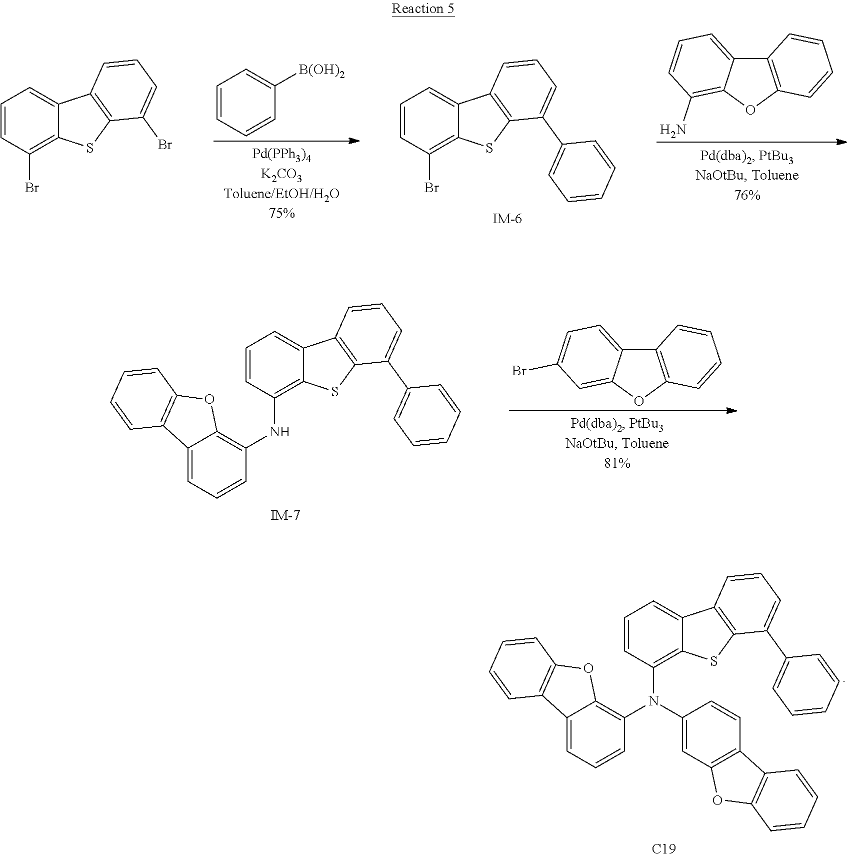

### Synthesis of Intermediate IM-6

Under an Ar atmosphere, to a 1000 mL, three-neck flask, 4,6-dibromodibenzothiophene (25.00 g, 73.1 mmol), phenylboronic acid (9.80 g, 1.1 equiv, 80.4 mmol), K₂CO₃ (30.30 g, 3.0 equiv, 219.3 mmol), Pd(PPh₃)₄ (4.22 g, 0.05 eq, 3.7 mmol), and 512 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-6 (18.60 g, yield 75%).

Through FAB-MS measurement, mass number m/z=339 was observed as an ion peak, and Intermediate IM-6 was identified.

### Synthesis of Intermediate IM-7

Under an Ar atmosphere, to a 500 mL, three-neck flask, IM-6 (15.00 g, 44.2 mmol), Pd(dba)₂ (0.76 g, 0.03 equiv, 1.3 mmol), NaOtBu (4.25 g, 1.0 equiv, 44.2 mmol), toluene (220 mL), 4-aminodibenzofuran (8.91 g, 1.1 equiv, 48.6 mmol) and tBu₃P (0.89 g, 0.1 equiv, 4.4 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-7 (14.84 g, yield 76%).

Through FAB-MS measurement, mass number m/z=441 was observed as an ion peak, and Intermediate IM-7 was identified.

### Synthesis of Compound C19

Under an Ar atmosphere, to a 300 mL, three-neck flask, IM-7 (10.00 g, 22.6 mmol), Pd(dba)₂ (0.39 g, 0.03 equiv, 0.7 mmol), NaOtBu (4.35 g, 2.0 equiv, 45.3 mmol), toluene (113 mL), 3-bromodibenzofuran (6.16 g, 1.1 equiv, 24.9 mmol) and tBu₃P (0.46 g, 0.1 equiv, 2.3 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound C19 of a solid (11.15 g, yield 81%).

Through FAB-MS measurement, mass number m/z=607 was observed as an ion peak, and Compound C19 was identified.

### 6. Synthesis of Compound C55

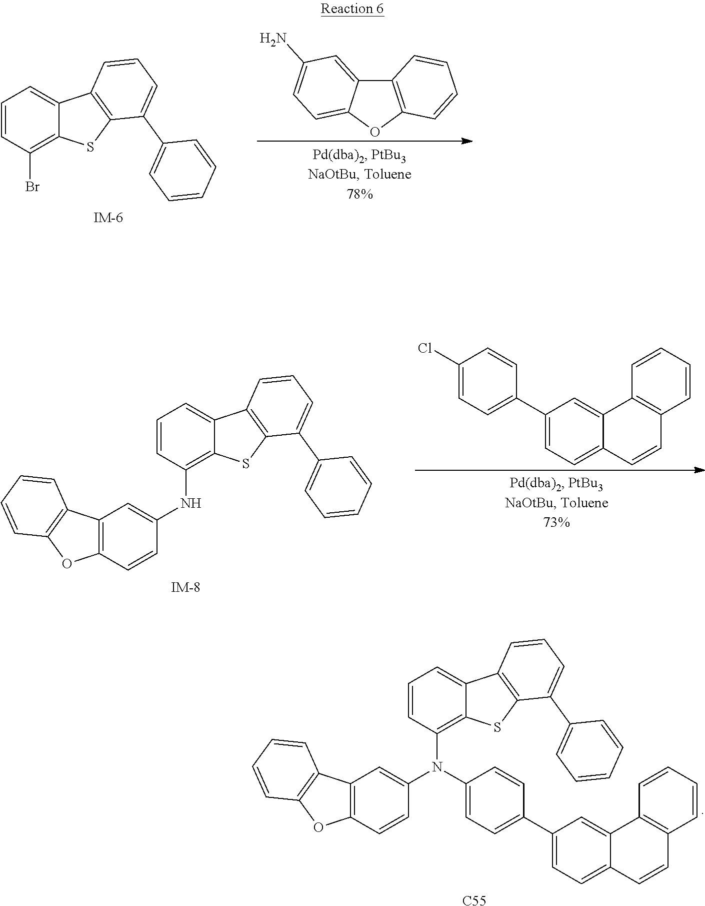

### Synthesis of Intermediate IM-8

Under an Ar atmosphere, to a 500 mL, three-neck flask, IM-6 (15.00 g, 44.2 mmol), Pd(dba)₂ (0.76 g, 0.03 equiv, 1.3 mmol), NaOtBu (4.25 g, 1.0 equiv, 44.2 mmol), toluene (220 mL), 2-aminodibenzofuran (8.91 g, 1.1 equiv, 48.6 mmol) and tBu₃P (0.89 g, 0.1 equiv, 4.4 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-8 (15.23 g, yield 78%).

Through FAB-MS measurement, mass number m/z=441 was observed as an ion peak, and Intermediate IM-8 was identified.

### Synthesis of Compound C55

Under an Ar atmosphere, to a 300 mL, three-neck flask, IM-8 (10.00 g, 22.6 mmol), Pd(dba)₂ (0.39 g, 0.03 equiv, 0.7 mmol), NaOtBu (4.35 g, 2.0 equiv, 45.3 mmol), toluene (113 mL), 3-(chlorophenyl)phenanthrene (7.19 g, 1.1 equiv, 24.9 mmol) and tBu₃P (0.46 g, 0.1 equiv, 2.3 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound C55 of a solid (11.47 g, yield 73%).

Through FAB-MS measurement, mass number m/z=693 was observed as an ion peak, and Compound C55 was identified.

### 7. Synthesis of Compound D40

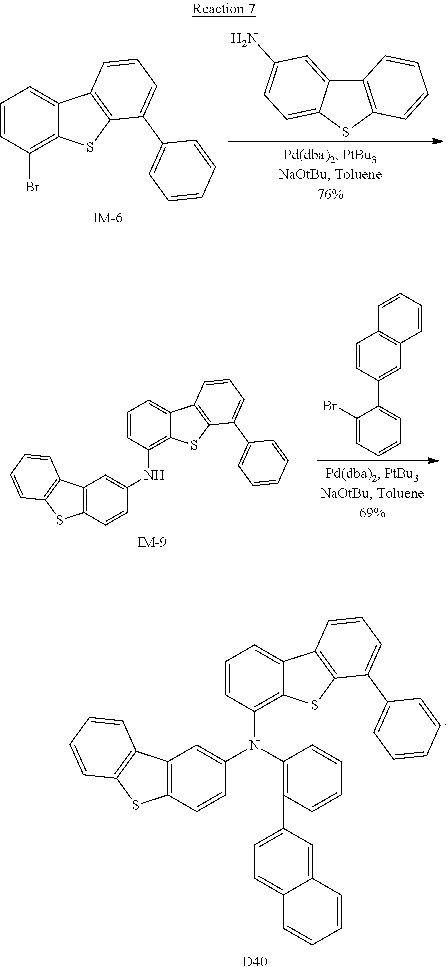

### Synthesis of Intermediate IM-9

Under an Ar atmosphere, to a 500 mL, three-neck flask, IM-6 (15.00 g, 44.2 mmol), Pd(dba)₂ (0.76 g, 0.03 equiv, 1.3 mmol), NaOtBu (4.25 g, 1.0 equiv, 44.2 mmol), toluene (220 mL), 2-aminodibenzothiophene (9.69 g, 1.1 equiv, 48.6 mmol) and tBu₃P (0.89 g, 0.1 equiv, 4.4 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-9 (15.38 g, yield 76%).

Through FAB-MS measurement, mass number m/z=457 was observed as an ion peak, and Intermediate IM-9 was identified.

### Synthesis of Compound D40

Under an Ar atmosphere, to a 300 mL, three-neck flask, IM-9 (10.00 g, 21.9 mmol), Pd(dba)₂ (0.38 g, 0.03 equiv, 0.7 mmol), NaOtBu (4.20 g, 2.0 equiv, 43.7 mmol), toluene (110 mL), 2-(2-bromophenyl)naphthalene (6.81 g, 1.1 equiv, 24.0 mmol) and tBu₃P (0.44 g, 0.1 equiv, 2.2 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound D40 of a solid (9.95 g, yield 69%).

Through FAB-MS measurement, mass number m/z=659 was observed as an ion peak, and Compound D40 was identified.

### 8. Synthesis of Compound D54

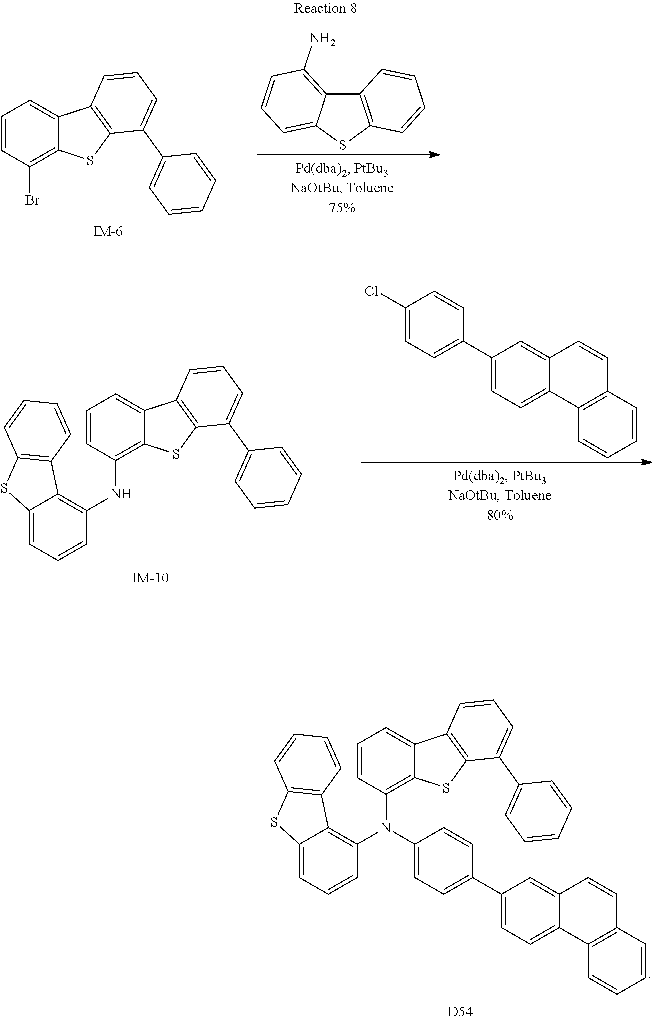

### Synthesis of Intermediate IM-10

Under an Ar atmosphere, to a 500 mL, three-neck flask, IM-6 (15.00 g, 44.2 mmol), Pd(dba)₂ (0.76 g, 0.03 equiv, 1.3 mmol), NaOtBu (4.25 g, 1.0 equiv, 44.2 mmol), toluene (220 mL), 1-aminodibenzothiophene (9.69 g, 1.1 equiv, 48.6 mmol) and tBu₃P (0.89 g, 0.1 equiv, 4.4 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-10 (15.17 g, yield 75%).

Through FAB-MS measurement, mass number m/z=457 was observed as an ion peak, and Intermediate IM-10 was identified.

### Synthesis of Compound D54

Under an Ar atmosphere, to a 300 mL, three-neck flask, IM-10 (10.00 g, 21.9 mmol), Pd(dba)₂ (0.38 g, 0.03 equiv, 0.7 mmol), NaOtBu (4.20 g, 2.0 equiv, 43.7 mmol), toluene (110 mL), 2-(4-chlorophenyl)phenanthrene (6.94 g, 1.1 equiv, 24.0 mmol) and tBu₃P (0.44 g, 0.1 equiv, 2.2 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound D54 of a solid (12.41 g, yield 80%).

Through FAB-MS measurement, mass number m/z=709 was observed as an ion peak, and Compound D54 was identified.

### 9. Synthesis of Compound E19

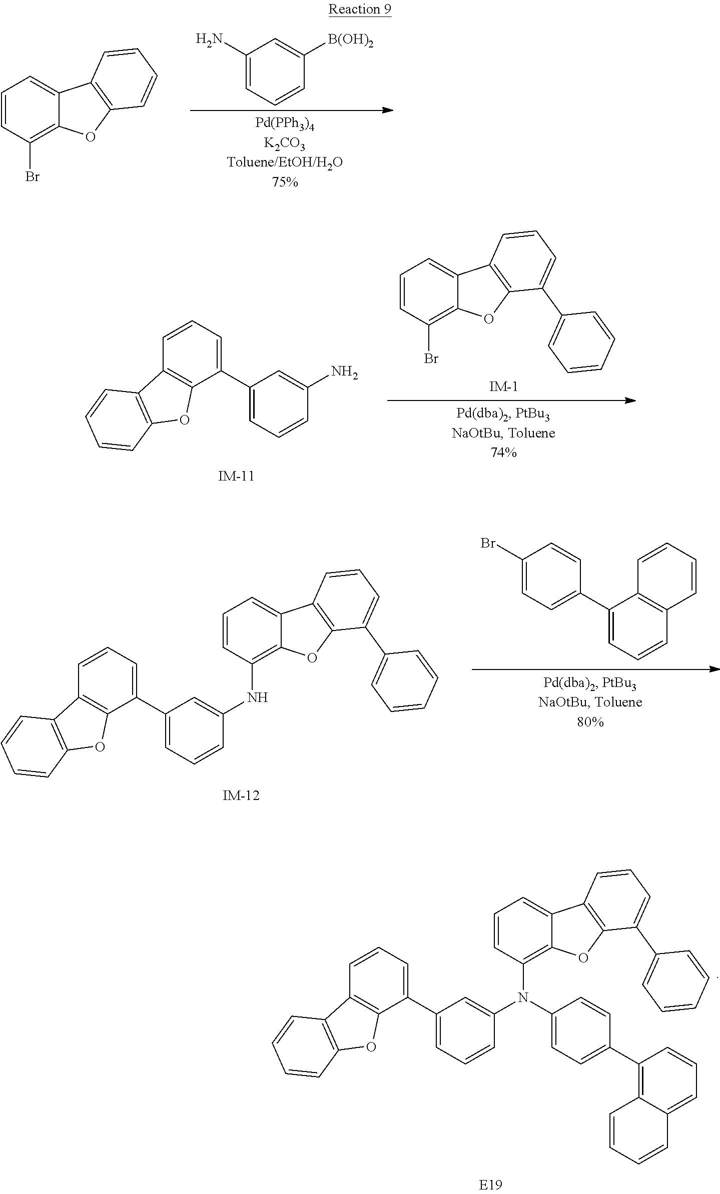

### Synthesis of Intermediate IM-11

Under an Ar atmosphere, to a 1000 mL, three-neck flask, 4-bromodibenzofuran (20.00 g, 80.9 mmol), (3-aminophenyl)boronic acid (12.19 g, 1.1 equiv, 89.0 mmol), K₂CO₃ (33.56 g, 3.0 equiv, 242.8 mmol), Pd(PPh₃)₄ (4.68 g, 0.05 eq, 4.0 mmol), and 566 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-11 (15.74 g, yield 75%).

Through FAB-MS measurement, mass number m/z=259 was observed as an ion peak, and Intermediate IM-11 was identified.

### Synthesis of Intermediate IM-12

Under an Ar atmosphere, to a 500 mL, three-neck flask, IM-11 (10.00 g, 38.6 mmol), Pd(dba)₂ (0.67 g, 0.03 equiv, 1.2 mmol), NaOtBu (3.71 g, 1.0 equiv, 38.6 mmol), toluene (192 mL), IM-1 (13.71 g, 1.1 equiv, 42.4 mmol) and tBu₃P (0.78 g, 0.1 equiv, 3.9 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-12 (14.31 g, yield 74%).

Through FAB-MS measurement, mass number m/z=501 was observed as an ion peak, and Intermediate IM-12 was identified.

### Synthesis of Compound E19

Under an Ar atmosphere, to a 300 mL, three-neck flask, IM-12 (10.00 g, 19.9 mmol), Pd(dba)₂ (0.34 g, 0.03 equiv, 0.6 mmol), NaOtBu (3.83 g, 2.0 equiv, 39.9 mmol), toluene (113 mL), 1-(4-bromophenyl)naphthalene (6.21 g, 1.1 equiv, 21.9 mmol) and tBu₃P (0.40 g, 0.1 equiv, 2.0 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound E19 of a solid (11.23 g, yield 80%).

Through FAB-MS measurement, mass number m/z=703 was observed as an ion peak, and Compound E19 was identified. 10. Synthesis of Compound E113

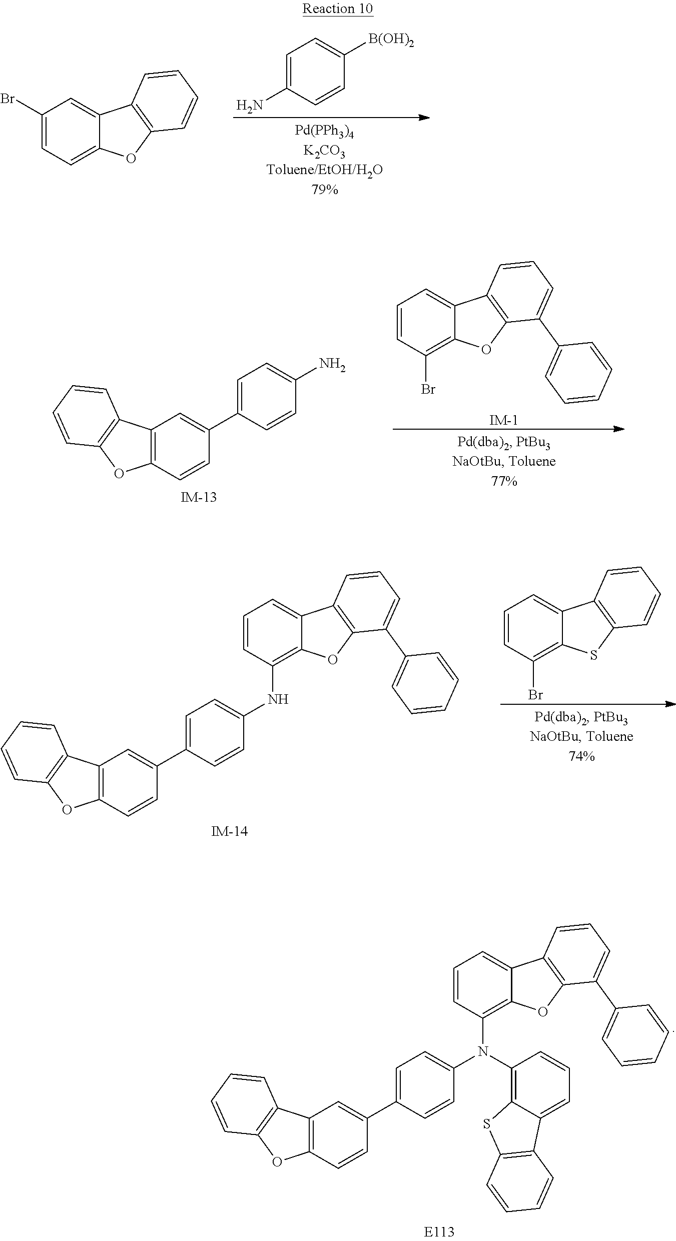

### Synthesis of Intermediate IM-13

Under an Ar atmosphere, to a 1000 mL, three-neck flask, 2-bromodibenzofuran (20.00 g, 80.9 mmol), (4-aminophenyl)boronic acid (12.19 g, 1.1 equiv, 89.0 mmol), K₂CO₃ (33.56 g, 3.0 equiv, 242.8 mmol), Pd(PPh₃)₄ (4.68 g, 0.05 eq, 4.0 mmol), and 566 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-13 (16.58 g, yield 79%).

Through FAB-MS measurement, mass number m/z=259 was observed as an ion peak, and Intermediate IM-13 was identified.

### Synthesis of Intermediate IM-14

Under an Ar atmosphere, to a 500 mL, three-neck flask, IM-14 (10.00 g, 38.6 mmol), Pd(dba)₂ (0.67 g, 0.03 equiv, 1.2 mmol), NaOtBu (3.71 g, 1.0 equiv, 38.6 mmol), toluene (192 mL), IM-1 (13.71 g, 1.1 equiv, 42.4 mmol) and tBu₃P (0.78 g, 0.1 equiv, 3.9 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-14 (14.89 g, yield 77%).

Through FAB-MS measurement, mass number m/z=501 was observed as an ion peak, and Intermediate IM-14 was identified.

### Synthesis of Compound E113

Under an Ar atmosphere, to a 300 mL, three-neck flask, IM-14 (10.00 g, 19.9 mmol), Pd(dba)₂ (0.34 g, 0.03 equiv, 0.6 mmol), NaOtBu (3.83 g, 2.0 equiv, 39.9 mmol), toluene (113 mL), 4-bromodibenzothiophene (5.77 g, 1.1 equiv, 21.9 mmol) and tBu₃P (0.40 g, 0.1 equiv, 2.0 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound E113 of a solid (10.19 g, yield 74%).

Through FAB-MS measurement, mass number m/z=683 was observed as an ion peak, and Compound E113 was identified.

### 11. Synthesis of Compound F22

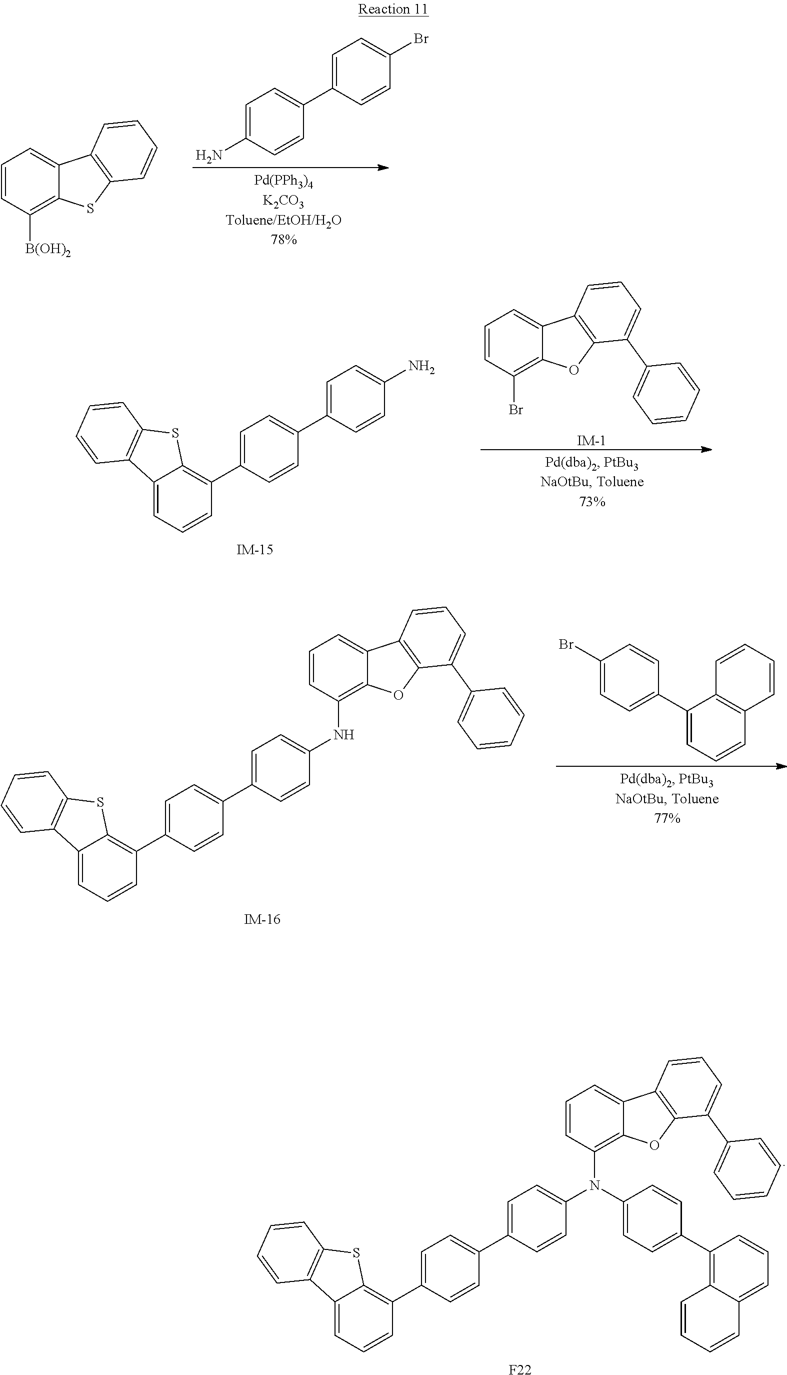

### Synthesis of Intermediate IM-15

Under an Ar atmosphere, to a 1000 mL, three-neck flask, dibenzothiophen-4-ylboronic acid (10.00 g, 43.8 mmol), 4′-bromo-[1,1′-biphenyl]-4-amine (11.96 g, 1.1 equiv, 48.2 mmol), K₂CO₃ (18.18 g, 3.0 equiv, 131.5 mmol), Pd(PPh₃)₄ (2.53 g, 0.05 eq, 2.2 mmol), and 307 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-15 (12.02 g, yield 78%).

Through FAB-MS measurement, mass number m/z=351 was observed as an ion peak, and Intermediate IM-15 was identified.

### Synthesis of Intermediate IM-16

Under an Ar atmosphere, to a 500 mL, three-neck flask, IM-15 (10.00 g, 28.5 mmol), Pd(dba)₂ (0.49 g, 0.03 equiv, 0.9 mmol), NaOtBu (2.73 g, 1.0 equiv, 28.5 mmol), toluene (142 mL), IM-1 (10.11 g, 1.1 equiv, 31.3 mmol) and tBu₃P (0.58 g, 0.1 equiv, 2.8 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-16 (12.33 g, yield 73%).

Through FAB-MS measurement, mass number m/z=593 was observed as an ion peak, and Intermediate IM-16 was identified.

### Synthesis of Compound F22

Under an Ar atmosphere, to a 300 mL, three-neck flask, IM-16 (10.00 g, 16.8 mmol), Pd(dba)₂ (0.29 g, 0.03 equiv, 0.5 mmol), NaOtBu (3.24 g, 2.0 equiv, 33.7 mmol), toluene (84 mL), 1-(4-bromophenyl)naphthalene (5.25 g, 1.1 equiv, 18.5 mmol) and tBu₃P (0.34 g, 0.1 equiv, 1.7 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound F22 of a solid (10.32 g, yield 74%).

Through FAB-MS measurement, mass number m/z=796 was observed as an ion peak, and Compound F22 was identified.

### 12. Synthesis of Compound F129

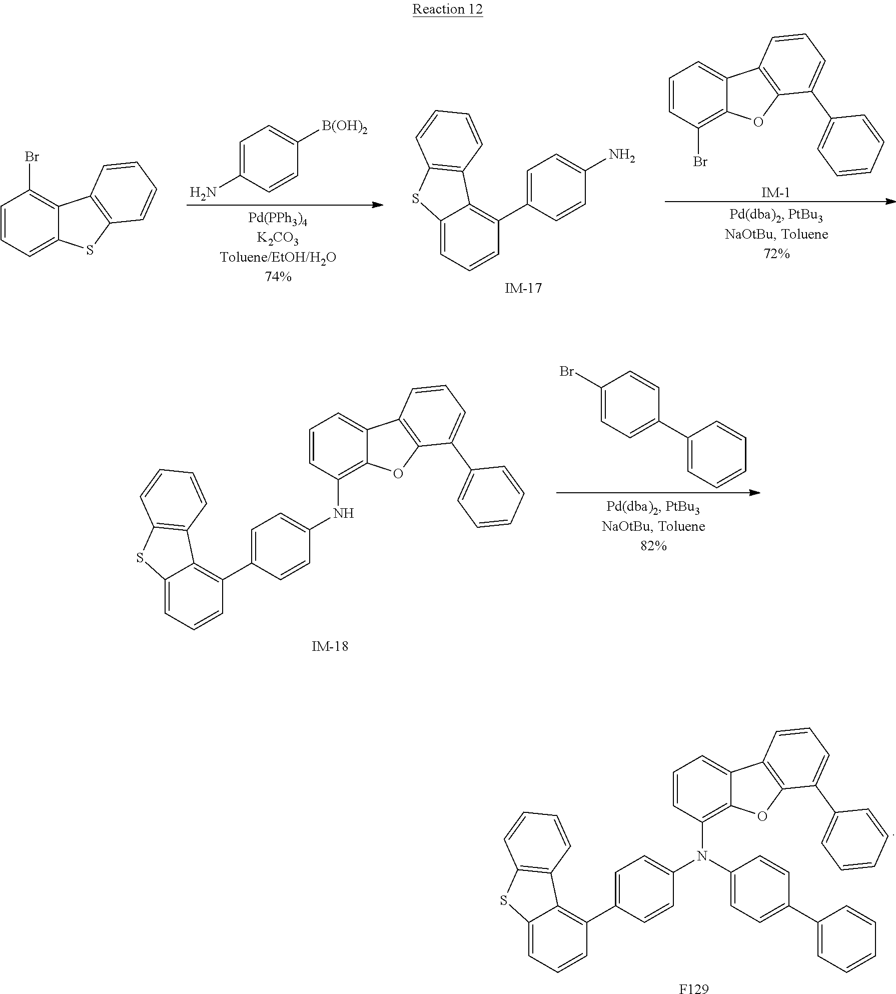

### Synthesis of Intermediate IM-17

Under an Ar atmosphere, to a 1000 mL, three-neck flask, 1-bromodibenzothiophene (20.00 g, 76.0 mmol), (4-aminophenyl)boronic acid (11.45 g, 1.1 equiv, 83.6 mmol), K₂CO₃ (31.51 g, 3.0 equiv, 228.0 mmol), Pd(PPh₃)₄ (4.39 g, 0.05 eq, 3.8 mmol), and 532 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-17 (15.49 g, yield 74%).

Through FAB-MS measurement, mass number m/z=275 was observed as an ion peak, and Intermediate IM-17 was identified.

### Synthesis of Intermediate IM-18

Under an Ar atmosphere, to a 500 mL, three-neck flask, IM-17 (10.00 g, 36.3 mmol), Pd(dba)₂ (0.63 g, 0.03 equiv, 1.1 mmol), NaOtBu (3.49 g, 1.0 equiv, 36.3 mmol), toluene (182 mL), IM-1 (12.91 g, 1.1 equiv, 39.9 mmol) and tBu₃P (0.73 g, 0.1 equiv, 3.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-18 (13.53 g, yield 72%).

Through FAB-MS measurement, mass number m/z=517 was observed as an ion peak, and Intermediate IM-18 was identified.

### Synthesis of Compound F129

Under an Ar atmosphere, to a 300 mL, three-neck flask, IM-18 (10.00 g, 19.3 mmol), Pd(dba)₂ (0.33 g, 0.03 equiv, 0.6 mmol), NaOtBu (3.71 g, 2.0 equiv, 38.6 mmol), toluene (97 mL), 4-bromobiphenyl (4.95 g, 1.1 equiv, 21.2 mmol) and tBu₃P (0.39 g, 0.1 equiv, 1.9 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound F129 of a solid (10.61 g, yield 82%).

Through FAB-MS measurement, mass number m/z=669 was observed as an ion peak, and Compound F129 was identified.

### 13. Synthesis of Compound G28

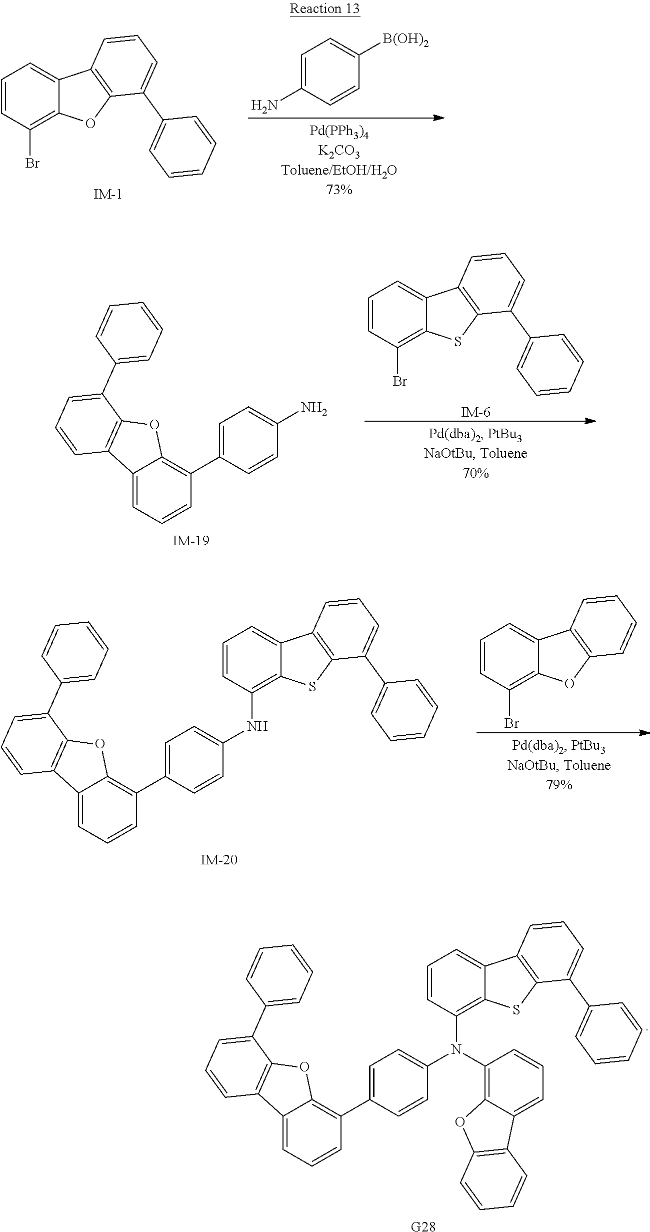

### Synthesis of Intermediate IM-19

Under an Ar atmosphere, to a 1000 mL, three-neck flask, IM-1 (20.00 g, 61.9 mmol), (4-aminophenyl)boronic acid (9.32 g, 1.1 equiv, 68.1 mmol), K₂CO₃ (25.66 g, 3.0 equiv, 185.6 mmol), Pd(PPh₃)₄ (3.58 g, 0.05 eq, 3.1 mmol), and 434 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-19 (15.15 g, yield 73%).

Through FAB-MS measurement, mass number m/z=351 was observed as an ion peak, and Intermediate IM-19 was identified.

### Synthesis of Intermediate IM-20

Under an Ar atmosphere, to a 500 mL, three-neck flask, IM-19 (10.00 g, 29.8 mmol), Pd(dba)₂ (0.51 g, 0.03 equiv, 0.9 mmol), NaOtBu (2.87 g, 1.0 equiv, 29.8 mmol), toluene (150 mL), IM-6 (11.13 g, 1.1 equiv, 32.8 mmol) and tBu₃P (0.60 g, 0.1 equiv, 3.0 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-20 (12.39 g, yield 70%).

Through FAB-MS measurement, mass number m/z=593 was observed as an ion peak, and Intermediate IM-20 was identified.

### Synthesis of Compound G28

Under an Ar atmosphere, to a 300 mL, three-neck flask, IM-20 (10.00 g, 16.8 mmol), Pd(dba)₂ (0.29 g, 0.03 equiv, 0.5 mmol), NaOtBu (3.24 g, 2.0 equiv, 33.7 mmol), toluene (84 mL), 4-bromodibenzofuran (4.58 g, 1.1 equiv, 18.5 mmol) and tBu₃P (0.34 g, 0.1 equiv, 1.7 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound G28 of a solid (10.11 g, yield 79%).

Through FAB-MS measurement, mass number m/z=759 was observed as an ion peak, and Compound G28 was identified.

### 14. Synthesis of Compound G51

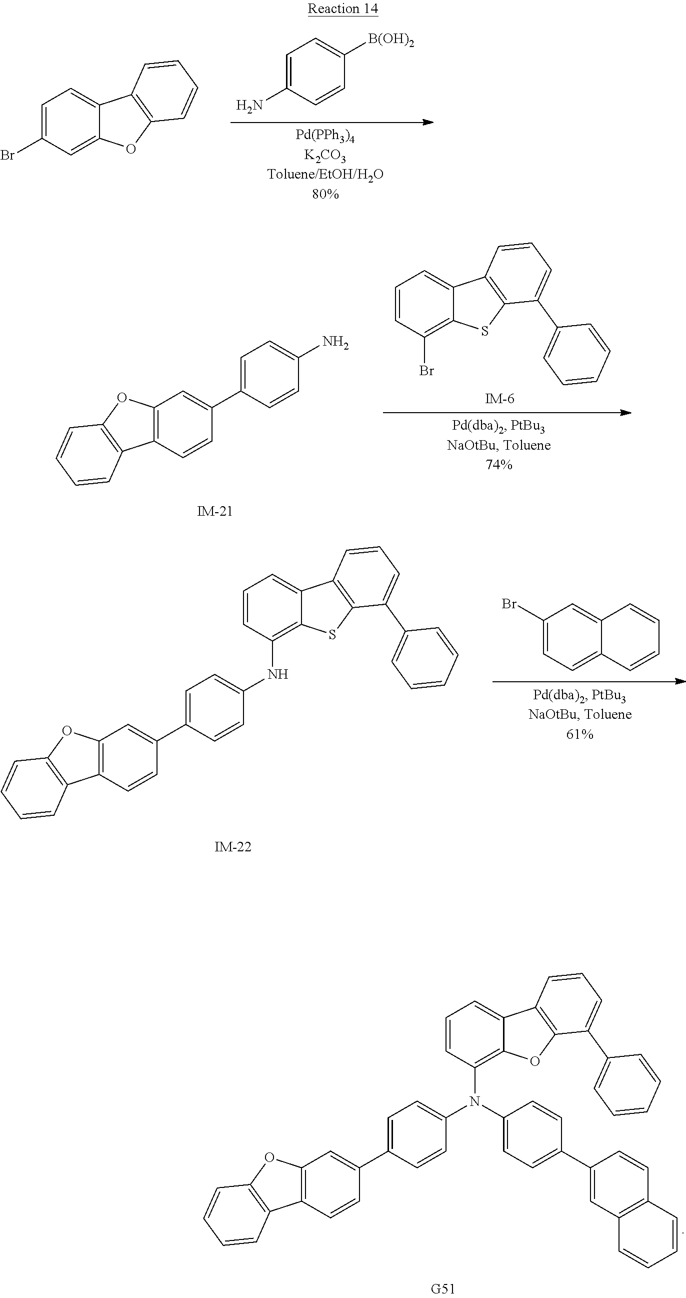

### Synthesis of Intermediate IM-21

Under an Ar atmosphere, to a 1000 mL, three-neck flask, 3-bromodibenzofuran (20.00 g, 80.9 mmol), (4-aminophenyl)boronic acid (12.19 g, 1.1 equiv, 89.0 mmol), K₂CO₃ (33.56 g, 3.0 equiv, 242.8 mmol), Pd(PPh₃)₄ (4.68 g, 0.05 eq, 4.0 mmol), and 566 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-21 (16.79 g, yield 80%).

Through FAB-MS measurement, mass number m/z=259 was observed as an ion peak, and Intermediate IM-21 was identified.

### Synthesis of Intermediate IM-22

Under an Ar atmosphere, to a 500 mL, three-neck flask, IM-21 (10.00 g, 38.6 mmol), Pd(dba)₂ (0.67 g, 0.03 equiv, 1.2 mmol), NaOtBu (3.71 g, 1.0 equiv, 38.6 mmol), toluene (192 mL), IM-6 (14.39 g, 1.1 equiv, 42.4 mmol) and tBu₃P (0.78 g, 0.1 equiv, 3.9 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-22 (14.77 g, yield 74%).

Through FAB-MS measurement, mass number m/z=517 was observed as an ion peak, and Intermediate IM-22 was identified.

### Synthesis of Compound G51

Under an Ar atmosphere, to a 300 mL, three-neck flask, IM-22 (10.00 g, 19.3 mmol), Pd(dba)₂ (0.33 g, 0.03 equiv, 0.6 mmol), NaOtBu (3.71 g, 2.0 equiv, 38.6 mmol), toluene (97 mL), 2-bromonaphthalene (4.40 g, 1.1 equiv, 21.2 mmol) and tBu₃P (0.39 g, 0.1 equiv, 1.9 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound G51 of a solid (10.07 g, yield 81%).

Through FAB-MS measurement, mass number m/z=643 was observed as an ion peak, and Compound G51 was identified.

### 15. Synthesis of Compound H1

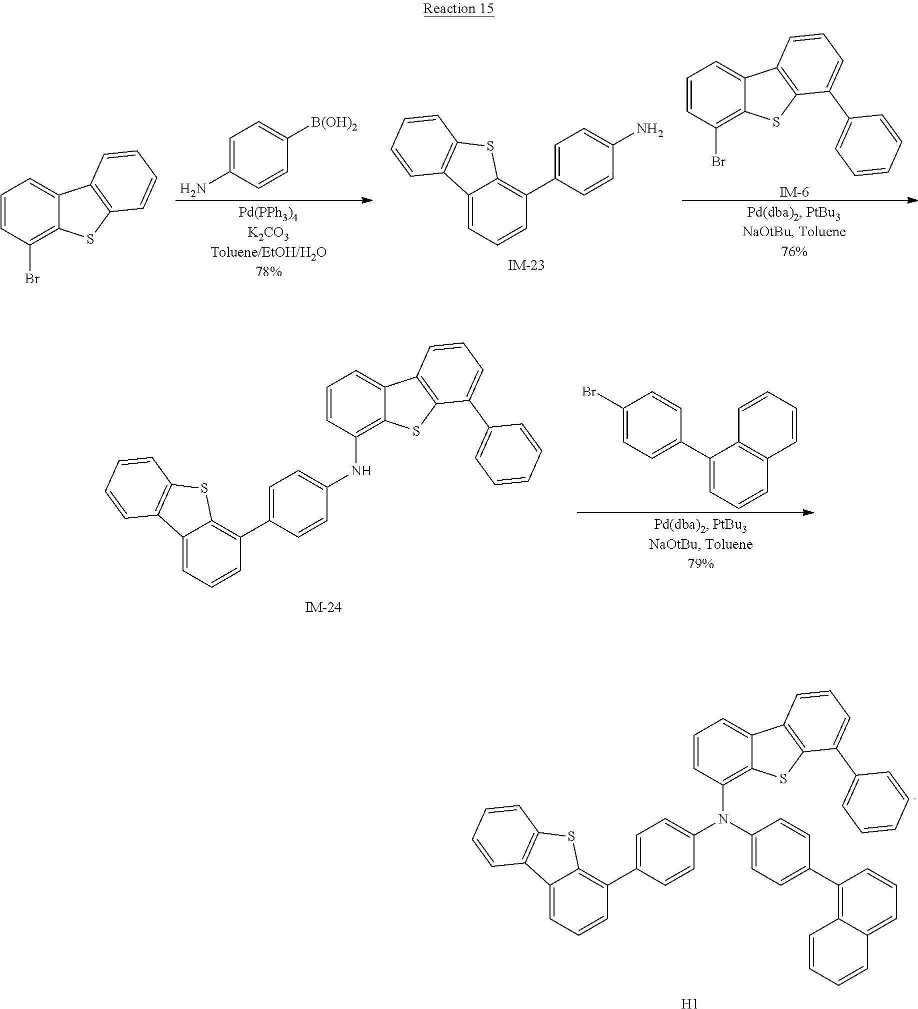

### Synthesis of Intermediate IM-23

Under an Ar atmosphere, to a 1000 mL, three-neck flask, 4-bromodibenzothiophene (20.00 g, 76.0 mmol), (4-aminophenyl)boronic acid (11.45 g, 1.1 equiv, 83.6 mmol), K₂CO₃ (31.51 g, 3.0 equiv, 228.0 mmol), Pd(PPh₃)₄ (4.39 g, 0.05 eq, 3.8 mmol), and 532 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-23 (16.32 g, yield 78%).

Through FAB-MS measurement, mass number m/z=275 was observed as an ion peak, and Intermediate IM-23 was identified.

### Synthesis of Intermediate IM-24

Under an Ar atmosphere, to a 500 mL, three-neck flask, IM-23 (10.00 g, 36.3 mmol), Pd(dba)₂ (0.63 g, 0.03 equiv, 1.1 mmol), NaOtBu (3.49 g, 1.0 equiv, 36.3 mmol), toluene (182 mL), IM-6 (13.55 g, 1.1 equiv, 39.9 mmol) and tBu₃P (0.73 g, 0.1 equiv, 3.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-24 (14.73 g, yield 76%).

Through FAB-MS measurement, mass number m/z=533 was observed as an ion peak, and Intermediate IM-24 was identified.

### Synthesis of Compound H1

Under an Ar atmosphere, to a 300 mL, three-neck flask, IM-24 (10.00 g, 18.7 mmol), Pd(dba)₂ (0.32 g, 0.03 equiv, 0.6 mmol), NaOtBu (3.60 g, 2.0 equiv, 37.4 mmol), toluene (94 mL), 1-(4-bromophenyl)naphthalene (5.84 g, 1.1 equiv, 20.6 mmol) and tBu₃P (0.38 g, 0.1 equiv, 1.9 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound H1 of a solid (10.89 g, yield 79%).

Through FAB-MS measurement, mass number m/z=735 was observed as an ion peak, and Compound H1 was identified.

### 16. Synthesis of Compound H88

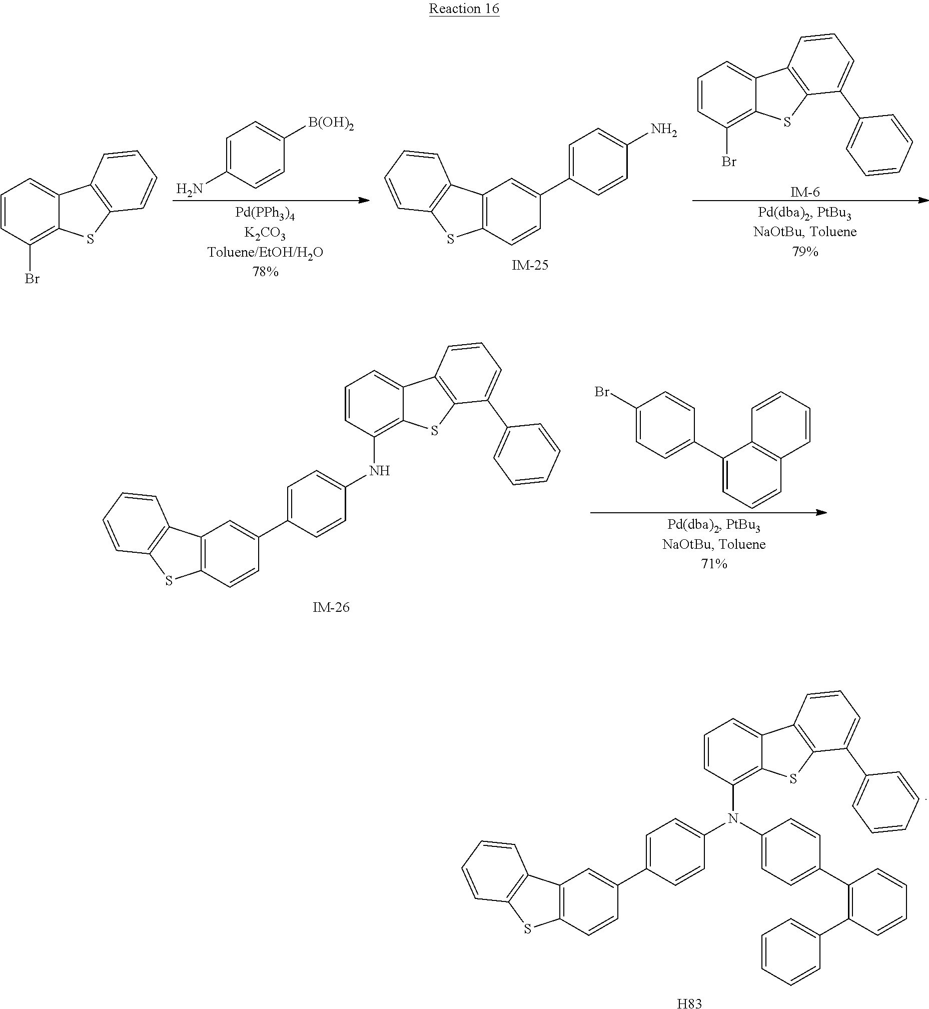

### Synthesis of Intermediate IM-25

Under an Ar atmosphere, to a 1000 mL, three-neck flask, 2-bromodibenzothiophene (20.00 g, 76.0 mmol), (4-aminophenyl)boronic acid (11.45 g, 1.1 equiv, 83.6 mmol), K₂CO₃ (31.51 g, 3.0 equiv, 228.0 mmol), Pd(PPh₃)₄ (4.39 g, 0.05 eq, 3.8 mmol), and 532 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-25 (15.70 g, yield 75%).

Through FAB-MS measurement, mass number m/z=275 was observed as an ion peak, and Intermediate IM-25 was identified.

### Synthesis of Intermediate IM-26

Under an Ar atmosphere, to a 500 mL, three-neck flask, IM-25 (10.00 g, 36.3 mmol), Pd(dba)₂ (0.63 g, 0.03 equiv, 1.1 mmol), NaOtBu (3.49 g, 1.0 equiv, 36.3 mmol), toluene (182 mL), IM-6 (13.55 g, 1.1 equiv, 39.9 mmol) and tBu₃P (0.73 g, 0.1 equiv, 3.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-26 (15.31 g, yield 79%).

Through FAB-MS measurement, mass number m/z=533 was observed as an ion peak, and Intermediate IM-26 was identified.

### Synthesis of Compound H88

Under an Ar atmosphere, to a 300 mL, three-neck flask, IM-26 (10.00 g, 18.7 mmol), Pd(dba)₂ (0.32 g, 0.03 equiv, 0.6 mmol), NaOtBu (3.60 g, 2.0 equiv, 37.4 mmol), toluene (94 mL), 4-bromo-1,1′:2′,1″-terphenyl (6.37 g, 1.1 equiv, 20.6 mmol) and tBu₃P (0.38 g, 0.1 equiv, 1.9 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound H88 of a solid (10.14 g, yield 71%).

Through FAB-MS measurement, mass number m/z=762 was observed as an ion peak, and Compound H88 was identified.

### 17. Synthesis of Compound I3

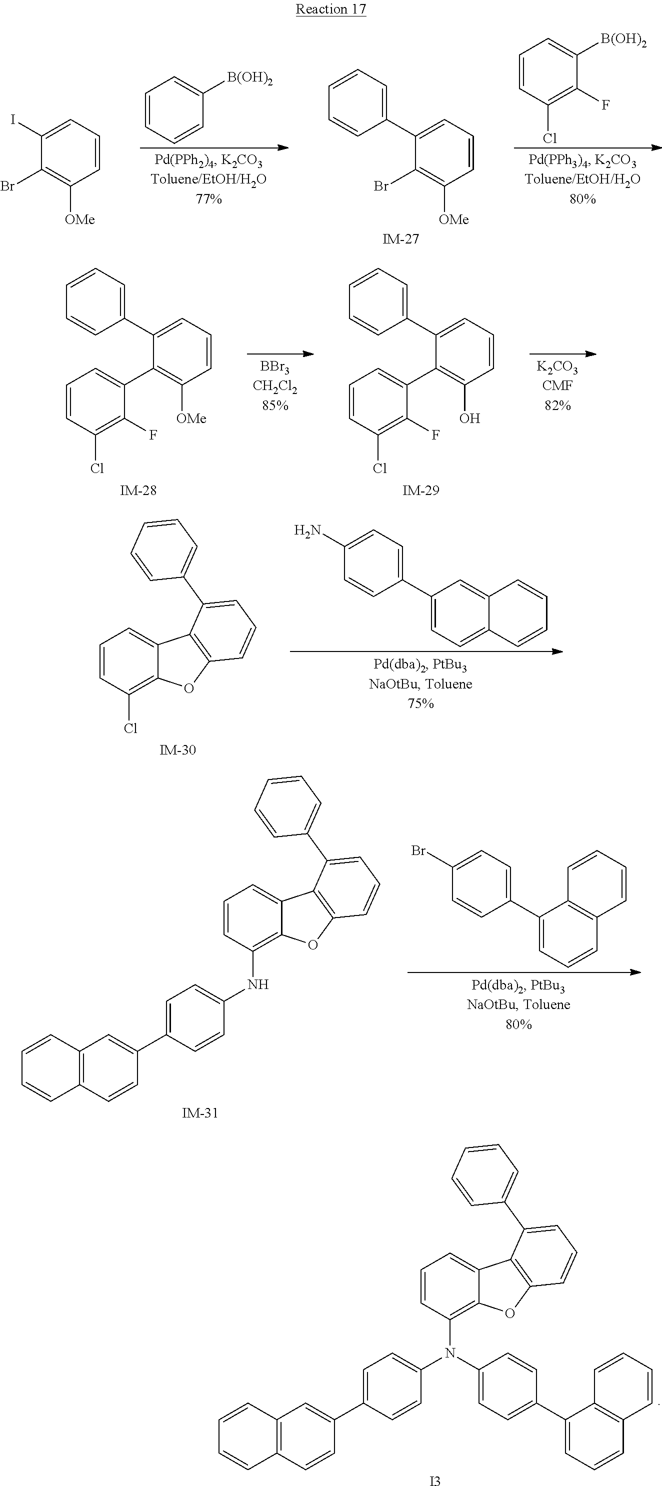

### Synthesis of Intermediate IM-27

Under an Ar atmosphere, to a 2000 mL, three-neck flask, 2-bromo-1-iodo-3-methoxybenzene (50.00 g, 159.8 mmol), phenylboronic acid (21.43 g, 1.1 equiv, 175.8 mmol), K₂CO₃ (66.25 g, 3.0 equiv, 479.3 mmol), Pd(PPh₃)₄ (9.23 g, 0.05 eq, 8.0 mmol), and 1118 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-27 (32.37 g, yield 77%).

Through FAB-MS measurement, mass number m/z=263 was observed as an ion peak, and Intermediate IM-27 was identified.

### Synthesis of Intermediate IM-28

Under an Ar atmosphere, to a 2000 mL, three-neck flask, IM-27 (30.00 g, 114.0 mmol), (3-chloro-2-fluorophenyl)boronic acid (21.87 g, 1.1 equiv, 125.4 mmol), K₂CO₃ (47.27 g, 3.0 equiv, 342.0 mmol), Pd(PPh₃)₄ (6.59 g, 0.05 eq, 5.7 mmol), and 798 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-28 (28.57 g, yield 80%).

Through FAB-MS measurement, mass number m/z=312 was observed as an ion peak, and Intermediate IM-28 was identified.

### Synthesis of Intermediate IM-29

Under an Ar atmosphere, to a 1000 mL, three-neck flask, IM-28 (25.00 g, 79.9 mmol), CH₂Cl₂ (266 mL) and a CH₂Cl₂ solution of 1 M BBr₃ (240 mL, 3.0 equiv, 240 mmol) were added in order, and stirred at room temperature for about 24 hours. The reaction solution was neutralized with a saturated NaHCO₃ aqueous solution and extracted with CH₂Cl₂. An organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-29 (20.30 g, yield 85%).

Through FAB-MS measurement, mass number m/z=298 was observed as an ion peak, and Intermediate IM-29 was identified.

### Synthesis of Intermediate IM-30

Under an Ar atmosphere, to a 500 mL, three-neck flask, IM-29 (18.00 g, 60.3 mmol), DMF (302 mL) and K₂CO₃ (33.31 g, 4 equiv, 241.0 mmol) were added in order, and heated to about 140° C. and stirred. After cooling to room temperature, H₂O was added to the reaction solution, and an organic layer was extracted with toluene, washed with a saturated saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-30 (13.77 g, yield 82%).

Through FAB-MS measurement, mass number m/z=278 was observed as an ion peak, and Intermediate IM-30 was identified.

### Synthesis of Intermediate IM-31

Under an Ar atmosphere, to a 500 mL, three-neck flask, IM-30 (10.00 g, 35.9 mmol), Pd(dba)₂ (0.62 g, 0.03 equiv, 1.1 mmol), NaOtBu (3.45 g, 1.0 equiv, 35.9 mmol), toluene (180 mL), 4-(naphthalen-2-yl)aniline (8.65 g, 1.1 equiv, 39.5 mmol) and tBu₃P (0.73 g, 0.1 equiv, 3.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-31 (12.42 g, yield 75%).

Through FAB-MS measurement, mass number m/z=461 was observed as an ion peak, and Intermediate IM-31 was identified.

### Synthesis of Compound I3

Under an Ar atmosphere, to a 300 mL, three-neck flask, IM-31 (8.00 g, 17.3 mmol), Pd(dba)₂ (0.30 g, 0.03 equiv, 0.5 mmol), NaOtBu (3.33 g, 2.0 equiv, 34.7 mmol), toluene (87 mL), 1-(4-bromophenyl)naphthalene (5.40 g, 1.1 equiv, 19.1 mmol) and tBu₃P (0.35 g, 0.1 equiv, 1.7 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound I3 of a solid (9.20 g, yield 80%).

Through FAB-MS measurement, mass number m/z=663 was observed as an ion peak, and Compound I3 was identified.

### 18. Synthesis of Compound I10

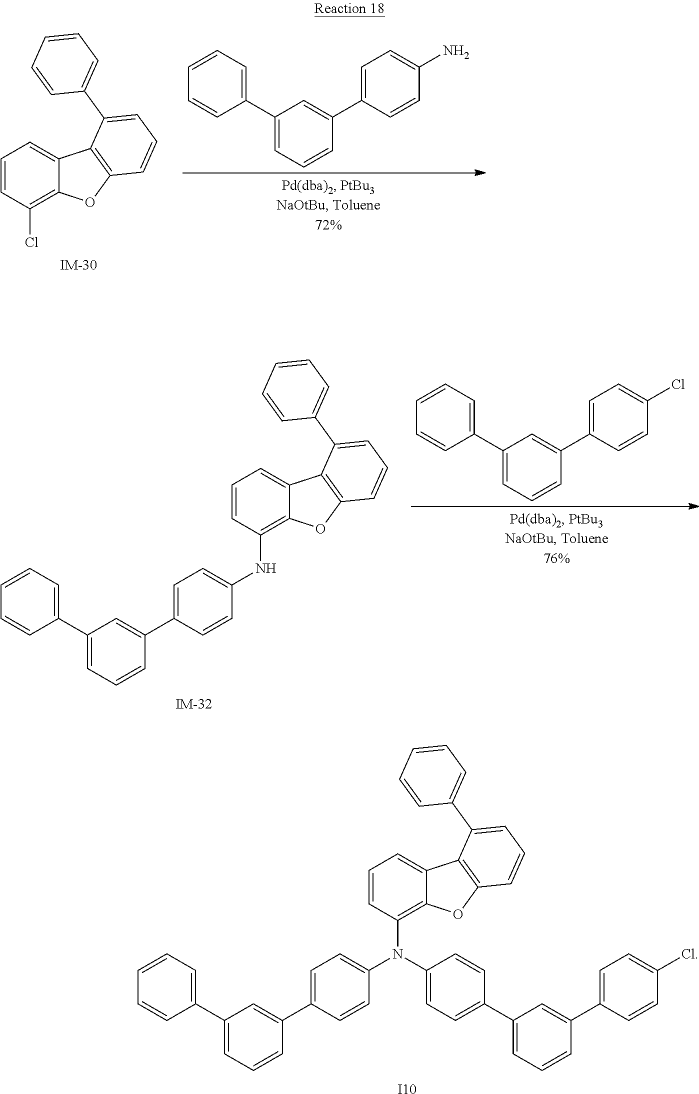

### Synthesis of Intermediate IM-32

Under an Ar atmosphere, to a 500 mL, three-neck flask, IM-30 (10.00 g, 35.9 mmol), Pd(dba)₂ (0.62 g, 0.03 equiv, 1.1 mmol), NaOtBu (3.45 g, 1.0 equiv, 35.9 mmol), toluene (180 mL), [1,1′:3′,1″-terphenyl]-4-amine (9.68 g, 1.1 equiv, 39.5 mmol) and tBu₃P (0.73 g, 0.1 equiv, 3.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-32 (12.59 g, yield 72%).

Through FAB-MS measurement, mass number m/z=487 was observed as an ion peak, and Intermediate IM-32 was identified.

### Synthesis of Compound I10

Under an Ar atmosphere, to a 300 mL, three-neck flask, IM-32 (8.00 g, 16.4 mmol), Pd(dba)₂ (0.28 g, 0.03 equiv, 0.5 mmol), NaOtBu (3.15 g, 2.0 equiv, 32.8 mmol), toluene (82 mL), 4-chloro-1,1′:3′,1″-terphenyl (4.78 g, 1.1 equiv, 18.0 mmol) and tBu₃P (0.33 g, 0.1 equiv, 1.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound I10 of a solid (8.93 g, yield 76%).

Through FAB-MS measurement, mass number m/z=715 was observed as an ion peak, and Compound I10 was identified. 19. Synthesis of Compound J15

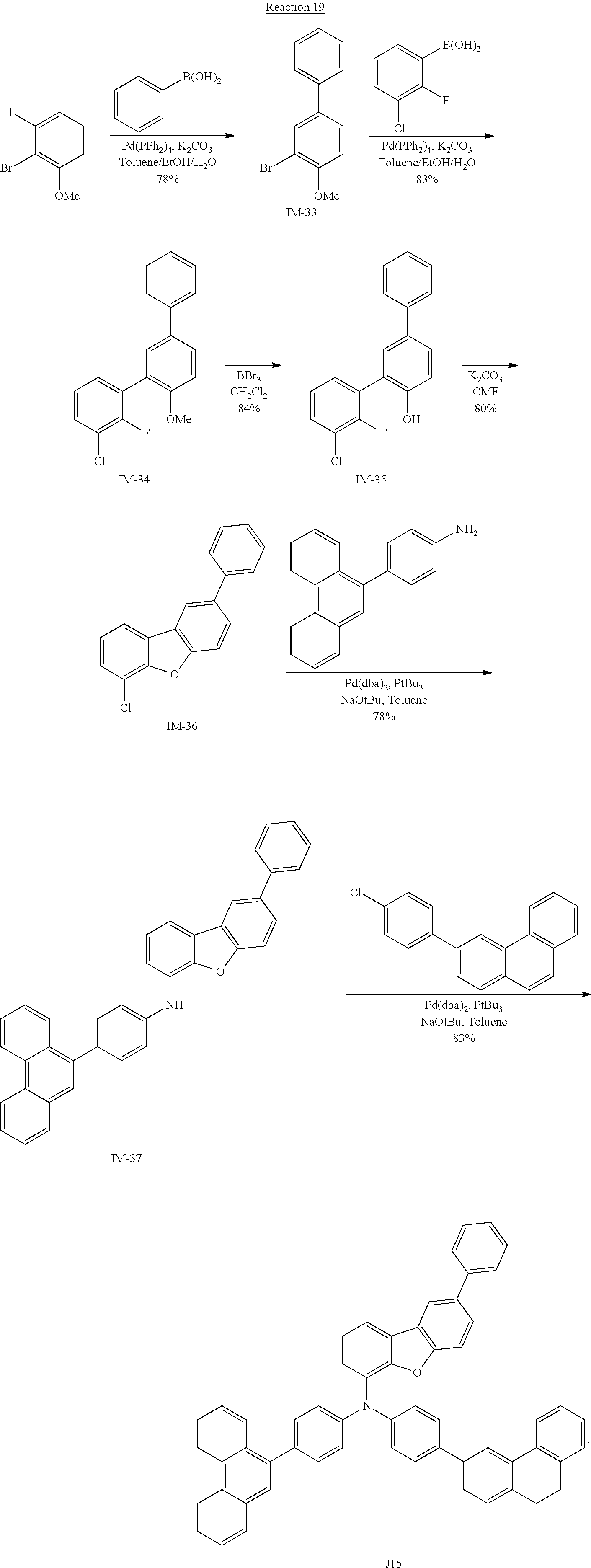

### Synthesis of Intermediate IM-33

Under an Ar atmosphere, to a 2000 mL, three-neck flask, 2-bromo-4-iodo-1-methoxybenzene (50.00 g, 159.8 mmol), phenylboronic acid (21.43 g, 1.1 equiv, 175.8 mmol), K₂CO₃ (66.25 g, 3.0 equiv, 479.3 mmol), Pd(PPh₃)₄ (9.23 g, 0.05 eq, 8.0 mmol), and 1118 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-33 (32.79 g, yield 78%).

Through FAB-MS measurement, mass number m/z=263 was observed as an ion peak, and Intermediate IM-33 was identified.

### Synthesis of Intermediate IM-34

Under an Ar atmosphere, to a 2000 mL, three-neck flask, IM-33 (30.00 g, 114.0 mmol), (3-chloro-2-fluorophenyl)boronic acid (21.87 g, 1.1 equiv, 125.4 mmol), K₂CO₃ (47.27 g, 3.0 equiv, 342.0 mmol), Pd(PPh₃)₄ (6.59 g, 0.05 eq, 5.7 mmol), and 798 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-34 (29.60 g, yield 83%).

Through FAB-MS measurement, mass number m/z=312 was observed as an ion peak, and Intermediate IM-34 was identified.

### Synthesis of Intermediate IM-35

Under an Ar atmosphere, to a 1000 mL, three-neck flask, IM-34 (25.00 g, 79.9 mmol), CH₂Cl₂ (266 mL) and a CH₂Cl₂ solution of 1 M BBr₃ (240 mL, 3.0 equiv, 240 mmol) were added in order, and stirred at room temperature for about 24 hours. The reaction solution was neutralized with a saturated NaHCO₃ aqueous solution and extracted with CH₂Cl₂. An organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-35 (20.06 g, yield 84%).

Through FAB-MS measurement, mass number m/z=298 was observed as an ion peak, and Intermediate IM-35 was identified.

### Synthesis of Intermediate IM-36

Under an Ar atmosphere, to a 500 mL, three-neck flask, IM-35 (18.00 g, 60.3 mmol), DMF (302 mL) and K₂CO₃ (33.31 g, 4 equiv, 241.0 mmol) were added in order, and heated to about 140° C. and stirred. After cooling to room temperature, H₂O was added to the reaction solution, and an organic layer was extracted with toluene, washed with a saturated saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-36 (13.44 g, yield 80%).

Through FAB-MS measurement, mass number m/z=278 was observed as an ion peak, and Intermediate IM-36 was identified.

### Synthesis of Intermediate IM-37

Under an Ar atmosphere, to a 500 mL, three-neck flask, IM-36 (10.00 g, 35.9 mmol), Pd(dba)₂ (0.62 g, 0.03 equiv, 1.1 mmol), NaOtBu (3.45 g, 1.0 equiv, 35.9 mmol), toluene (180 mL), 4-(phenanthren-2-yl)aniline (10.63 g, 1.1 equiv, 39.5 mmol) and tBu₃P (0.73 g, 0.1 equiv, 3.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-37 (14.32 g, yield 78%).

Through FAB-MS measurement, mass number m/z=511 was observed as an ion peak, and Intermediate IM-37 was identified.

### Synthesis of Compound J15

Under an Ar atmosphere, to a 300 mL, three-neck flask, IM-37 (8.00 g, 15.6 mmol), Pd(dba)₂ (0.27 g, 0.03 equiv, 0.5 mmol), NaOtBu (3.01 g, 2.0 equiv, 31.3 mmol), toluene (78 mL), 3-(4-chlorophenyl)phenanthrene (5.21 g, 1.1 equiv, 17.2 mmol) and tBu₃P (0.32 g, 0.1 equiv, 1.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound J15 of a solid (10.40 g, yield 83%).

Through FAB-MS measurement, mass number m/z=763 was observed as an ion peak, and Compound J15 was identified.

### 20. Synthesis of Compound J18

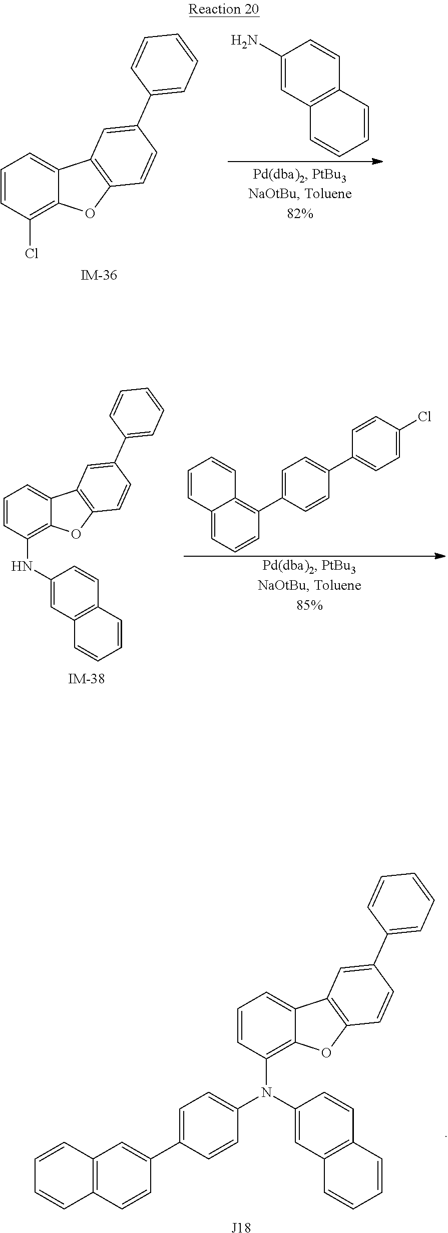

### Synthesis of Intermediate IM-38

Under an Ar atmosphere, to a 500 mL, three-neck flask, IM-36 (10.00 g, 35.9 mmol), Pd(dba)₂ (0.62 g, 0.03 equiv, 1.1 mmol), NaOtBu (3.45 g, 1.0 equiv, 35.9 mmol), toluene (180 mL), naphthalen-2-amine (5.34 g, 1.1 equiv, 39.5 mmol) and tBu₃P (0.73 g, 0.1 equiv, 3.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-38 (10.72 g, yield 82%).

Through FAB-MS measurement, mass number m/z=385 was observed as an ion peak, and Intermediate IM-38 was identified.

### Synthesis of Compound J18

Under an Ar atmosphere, to a 300 mL, three-neck flask, IM-38 (8.00 g, 20.8 mmol), Pd(dba)₂ (0.36 g, 0.03 equiv, 0.5 mmol), NaOtBu (3.99 g, 2.0 equiv, 41.5 mmol), toluene (108 mL), 1-[4′-chloro-(1,1′-biphenyl)-4-yl]naphthalene (7.19 g, 1.1 equiv, 22.8 mmol) and tBu₃P (0.42 g, 0.1 equiv, 2.1 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound J18 of a solid (11.71 g, yield 85%).

Through FAB-MS measurement, mass number m/z=663 was observed as an ion peak, and Compound J18 was identified.

### 21. Synthesis of Compound K6

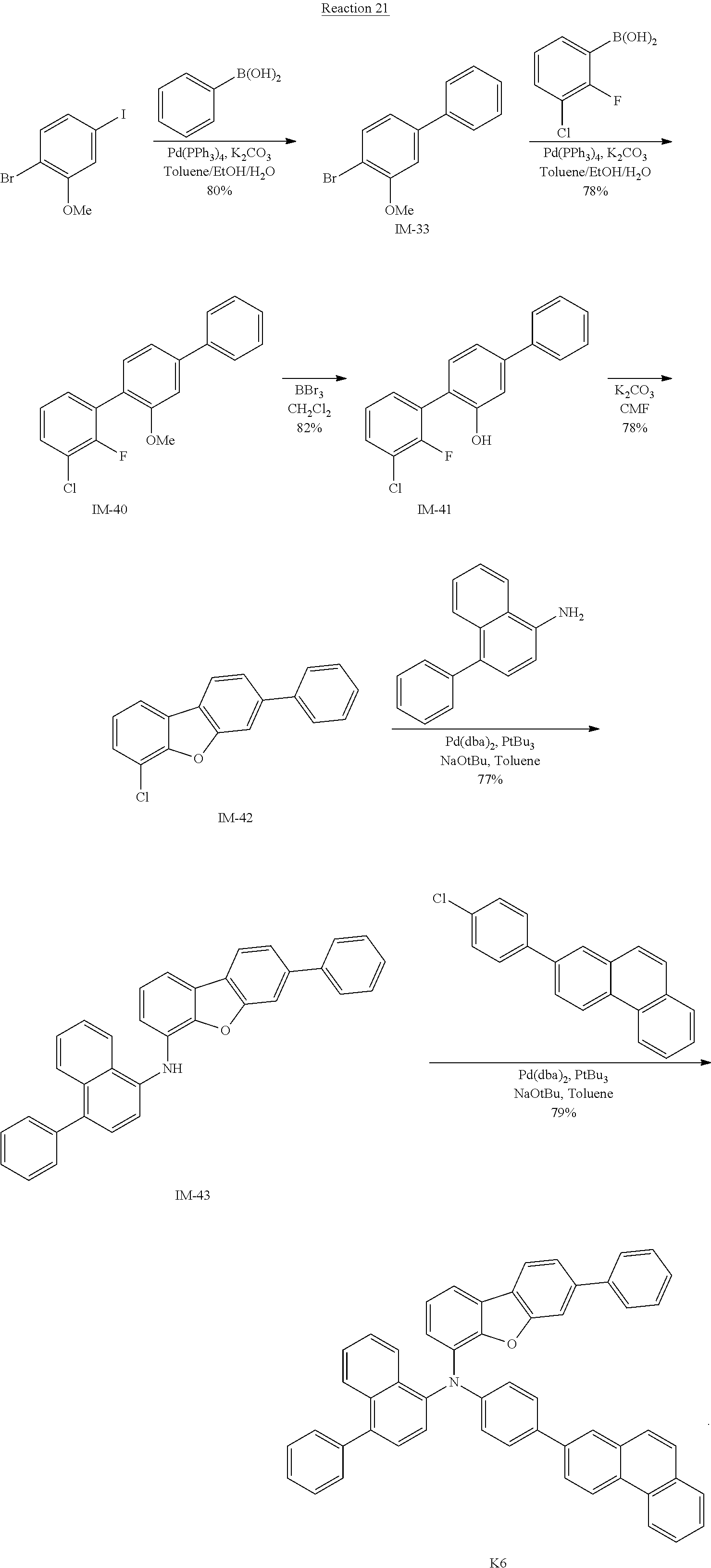

### Synthesis of Intermediate IM-39

Under an Ar atmosphere, to a 2000 mL, three-neck flask, 1-bromo-4-iodo-2-methoxybenzene (50.00 g, 159.8 mmol), phenylboronic acid (21.43 g, 1.1 equiv, 175.8 mmol), K₂CO₃ (66.25 g, 3.0 equiv, 479.3 mmol), Pd(PPh₃)₄ (9.23 g, 0.05 eq, 8.0 mmol), and 1118 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-39 (33.63 g, yield 80%).

Through FAB-MS measurement, mass number m/z=263 was observed as an ion peak, and Intermediate IM-39 was identified.

### Synthesis of Intermediate IM-40

Under an Ar atmosphere, to a 2000 mL, three-neck flask, IM-39 (30.00 g, 114.0 mmol), (3-chloro-2-fluorophenyl)boronic acid (21.87 g, 1.1 equiv, 125.4 mmol), K₂CO₃ (47.27 g, 3.0 equiv, 342.0 mmol), Pd(PPh₃)₄ (6.59 g, 0.05 eq, 5.7 mmol), and 798 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-40 (27.10 g, yield 76%).

Through FAB-MS measurement, mass number m/z=312 was observed as an ion peak, and Intermediate IM-40 was identified.

### Synthesis of Intermediate IM-41

Under an Ar atmosphere, to a 1000 mL, three-neck flask, IM-40 (25.00 g, 79.9 mmol), CH₂Cl₂ (266 mL) and a CH₂Cl₂ solution of 1 M BBr₃ (240 mL, 3.0 equiv, 240 mmol) were added in order, and stirred at room temperature for about 24 hours. The reaction solution was neutralized with a saturated NaHCO₃ aqueous solution and extracted with CH₂Cl₂. An organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-41 (19.58 g, yield 82%).

Through FAB-MS measurement, mass number m/z=298 was observed as an ion peak, and Intermediate IM-41 was identified.

### Synthesis of Intermediate IM-42

Under an Ar atmosphere, to a 500 mL, three-neck flask, IM-41 (18.00 g, 60.3 mmol), DMF (302 mL) and K₂CO₃ (33.31 g, 4 equiv, 241.0 mmol) were added in order, and heated to about 140° C. and stirred. After cooling to room temperature, H₂O was added to the reaction solution, and an organic layer was extracted with toluene, washed with a saturated saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-42 (13.10 g, yield 78%).

Through FAB-MS measurement, mass number m/z=278 was observed as an ion peak, and Intermediate IM-42 was identified.

### Synthesis of Intermediate IM-43

Under an Ar atmosphere, to a 500 mL, three-neck flask, IM-42 (10.00 g, 35.9 mmol), Pd(dba)₂ (0.62 g, 0.03 equiv, 1.1 mmol), NaOtBu (3.45 g, 1.0 equiv, 35.9 mmol), toluene (180 mL), 4-phenylnaphthalen-1-amine (8.65 g, 1.1 equiv, 39.5 mmol) and tBu₃P (0.73 g, 0.1 equiv, 3.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-43 (12.75 g, yield 77%).

Through FAB-MS measurement, mass number m/z=461 was observed as an ion peak, and Intermediate IM-43 was identified.

### Synthesis of Compound K6

Under an Ar atmosphere, to a 300 mL, three-neck flask, IM-43 (8.00 g, 17.3 mmol), Pd(dba)₂ (0.30 g, 0.03 equiv, 0.5 mmol), NaOtBu (3.33 g, 2.0 equiv, 34.7 mmol), toluene (87 mL), 2-(4-chlorophenyl)phenanthrene (5.51 g, 1.1 equiv, 19.1 mmol) and tBu₃P (0.35 g, 0.1 equiv, 1.7 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound K6 of a solid (9.77 g, yield 79%).

Through FAB-MS measurement, mass number m/z=713 was observed as an ion peak, and Compound K6 was identified.

### 22. Synthesis of Compound L1

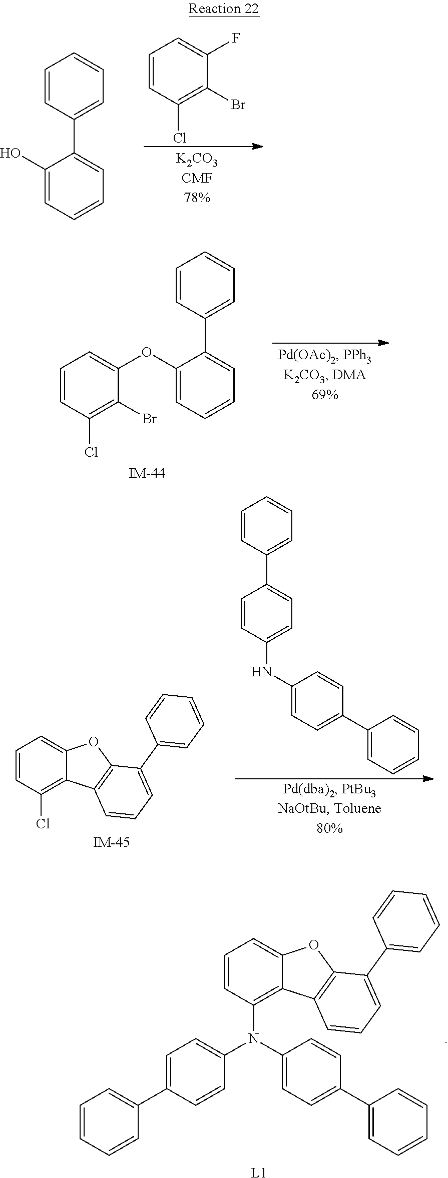

### Synthesis of Intermediate IM-44

Under an Ar atmosphere, to a 1000 mL, three-neck flask, [1,1′-biphenyl]-2-ol (20.00 g, 170.2 mmol), 2-bromo-1-chloro-3-fluorobenzene (49.22 g, 2 equiv, 235.0 mmol), DMF (588 mL) and K₂CO₃ (65.0 g, 4 equiv, 470.0 mmol) were added in order, and heated to about 140° C. and stirred. After cooling to room temperature, H₂O was added to the reaction solution, and an organic layer was extracted with toluene, washed with a saturated saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-44 (32.96 g, yield 78%).

Through FAB-MS measurement, mass number m/z=359 was observed as an ion peak, and Intermediate IM-44 was identified.

### Synthesis of Intermediate IM-45

Under an Ar atmosphere, to a 500 mL, three-neck flask, IM-44 (25.00 g, 69.5 mmol), Pd(OAc)₂ (0.78 g, 0.05 equiv, 3.5 mmol), K₂CO₃ (14.41 g, 1.5 equiv, 104.3 mmol), N,N-dimethylacetamide (DMA, 278 mL) and PPh₃ (1.82 g, 0.1 equiv, 7.0 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-45 (13.37 g, yield 69%).

Through FAB-MS measurement, mass number m/z=278 was observed as an ion peak, and Intermediate IM-45 was identified.

### Synthesis of Compound L1

Under an Ar atmosphere, to a 300 mL, three-neck flask, IM-45 (8.00 g, 28.7 mmol), Pd(dba)₂ (0.50 g, 0.03 equiv, 0.9 mmol), NaOtBu (5.52 g, 2.0 equiv, 57.4 mmol), toluene (144 mL), bis(4-biphenylyl)amine (10.15 g, 1.1 equiv, 31.6 mmol) and tBu₃P (0.58 g, 0.1 equiv, 2.9 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound L1 of a solid (12.94 g, yield 80%).

Through FAB-MS measurement, mass number m/z=563 was observed as an ion peak, and Compound L1 was identified.

### 23. Synthesis of Compound M5

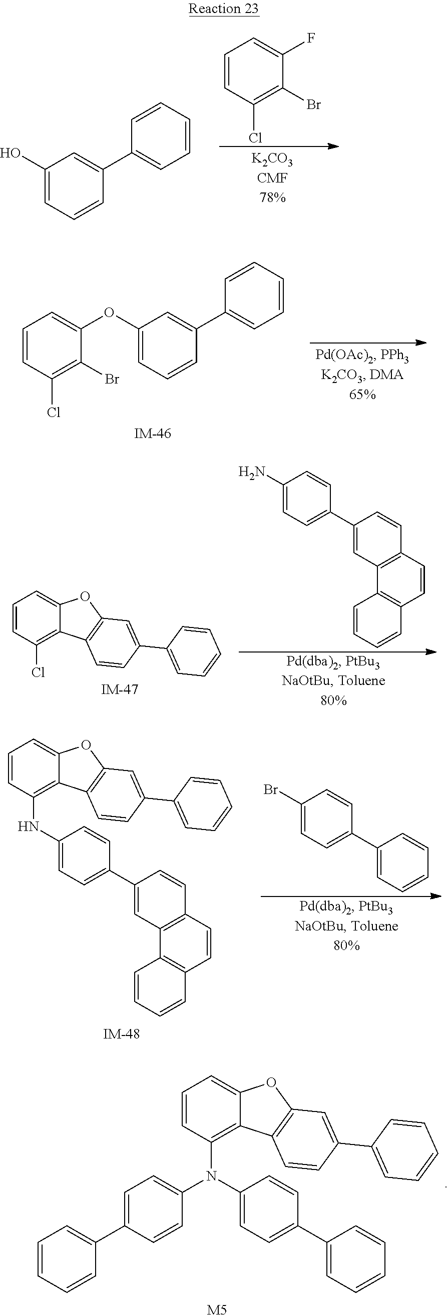

### Synthesis of Intermediate IM-46

Under an Ar atmosphere, to a 1000 mL, three-neck flask, [1,1′-biphenyl]-3-ol (20.00 g, 170.2 mmol), 2-bromo-1-chloro-3-fluorobenzene (49.22 g, 2 equiv, 235.0 mmol), DMF 588 mL and K₂CO₃ 65.0 g (4 equiv, 470.0 mmol) were added in order, and heated to about 140° C. and stirred. After cooling to room temperature, H₂O was added to the reaction solution, and an organic layer was extracted with toluene, washed with a saturated saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-46 (33.81 g, yield 80%).

Through FAB-MS measurement, mass number m/z=359 was observed as an ion peak, and Intermediate IM-47 was identified.

### Synthesis of Intermediate IM-47

Under an Ar atmosphere, to a 1000 mL, three-neck flask, IM-46 (25.00 g, 69.5 mmol), Pd(OAc)₂ (0.78 g, 0.05 equiv, 3.5 mmol), K₂CO₃ (14.41 g, 1.5 equiv, 104.3 mmol), N,N-dimethylacetamide (DMA, 278 mL) and PPh₃ (1.82 g, 0.1 equiv, 7.0 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-47 (12.59 g, yield 65%).

Through FAB-MS measurement, mass number m/z=278 was observed as an ion peak, and Intermediate IM-47 was identified.

### Synthesis of Intermediate IM-48

Under an Ar atmosphere, to a 500 mL, three-neck flask, IM-47 (10.00 g, 35.9 mmol), Pd(dba)₂ (0.62 g, 0.03 equiv, 1.1 mmol), NaOtBu (3.45 g, 1.0 equiv, 35.9 mmol), toluene (180 mL), 4-(phenanthren-3-yl)aniline (10.63 g, 1.1 equiv, 39.5 mmol) and tBu₃P (0.73 g, 0.1 equiv, 3.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-48 (14.68 g, yield 80%).

Through FAB-MS measurement, mass number m/z=511 was observed as an ion peak, and Intermediate IM-48 was identified.

### Synthesis of Compound M5

Under an Ar atmosphere, to a 300 mL, three-neck flask, IM-48 (8.00 g, 15.6 mmol), Pd(dba)₂ (0.27 g, 0.03 equiv, 0.5 mmol), NaOtBu (3.01 g, 2.0 equiv, 31.3 mmol), toluene (78 mL), 4-bromobiphenyl (4.01 g, 1.1 equiv, 17.2 mmol) and tBu₃P (0.32 g, 0.1 equiv, 1.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound M5 of a solid (8.30 g, yield 80%).

Through FAB-MS measurement, mass number m/z=663 was observed as an ion peak, and Compound M5 was identified.

### 24. Synthesis of Compound M20

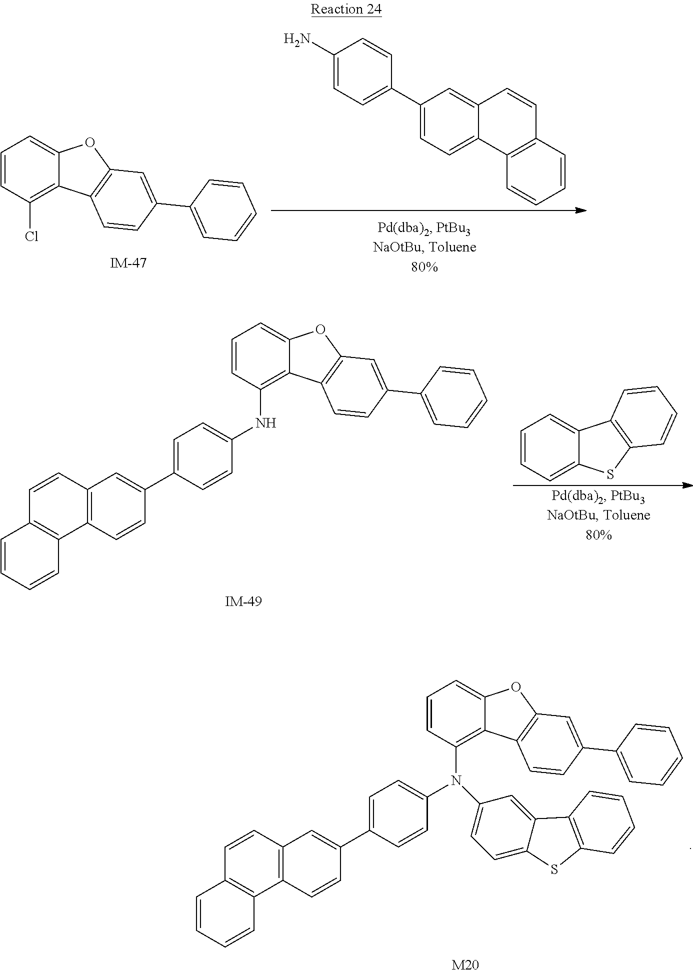

### Synthesis of Intermediate IM-49

Under an Ar atmosphere, to a 500 mL, three-neck flask, IM-47 (10.00 g, 35.9 mmol), Pd(dba)₂ (0.62 g, 0.03 equiv, 1.1 mmol), NaOtBu (3.45 g, 1.0 equiv, 35.9 mmol), toluene (180 mL), 4-(phenanthren-2-yl)aniline (10.63 g, 1.1 equiv, 39.5 mmol) and tBu₃P (0.73 g, 0.1 equiv, 3.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-49 (13.58 g, yield 74%).

Through FAB-MS measurement, mass number m/z=511 was observed as an ion peak, and Intermediate IM-49 was identified.

### Synthesis of Compound M20

Under an Ar atmosphere, to a 300 mL, three-neck flask, IM-49 (8.00 g, 15.6 mmol), Pd(dba)₂ (0.27 g, 0.03 equiv, 0.5 mmol), NaOtBu (3.01 g, 2.0 equiv, 31.3 mmol), toluene (78 mL), 2-bromodibenzothiophene (4.53 g, 1.1 equiv, 17.2 mmol) and tBu₃P (0.32 g, 0.1 equiv, 1.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound M20 of a solid (8.57 g, yield 79%).

Through FAB-MS measurement, mass number m/z=693 was observed as an ion peak, and Compound M20 was identified.

### 25. Synthesis of Compound N4

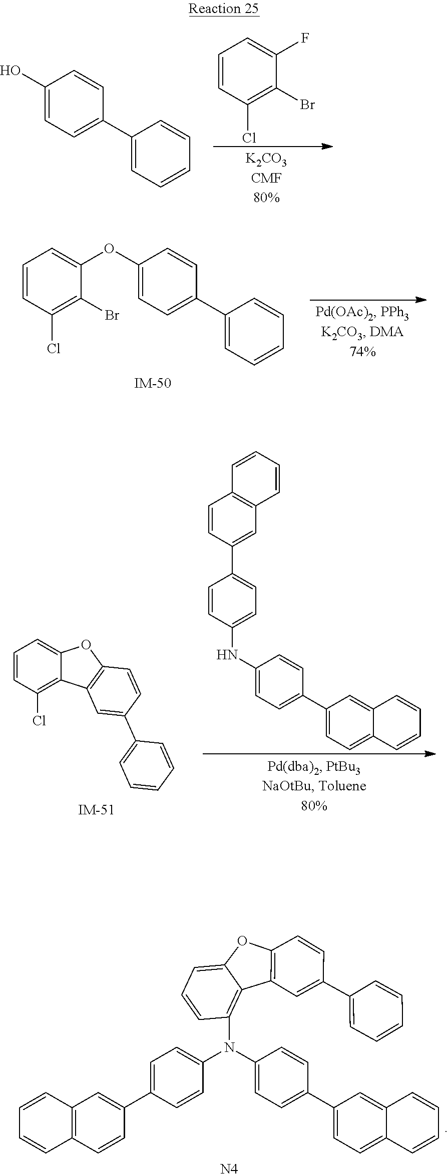

### Synthesis of Intermediate IM-50

Under an Ar atmosphere, to a 1000 mL, three-neck flask, [1,1′-biphenyl]-4-ol (20.00 g, 170.2 mmol), 2-bromo-1-chloro-3-fluorobenzene (49.22 g, 2 equiv, 235.0 mmol), DMF (588 mL) and K₂CO₃ (65.0 g, 4 equiv, 470.0 mmol) were added in order, and heated to about 140° C. and stirred. After cooling to room temperature, H₂O was added to the reaction solution, and an organic layer was extracted with toluene, washed with a saturated saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-50 (33.81 g, yield 80%).

Through FAB-MS measurement, mass number m/z=359 was observed as an ion peak, and Intermediate IM-50 was identified.

### Synthesis of Intermediate IM-51

Under an Ar atmosphere, to a 500 mL, three-neck flask, IM-50 (25.00 g, 69.5 mmol), Pd(OAc)₂ (0.78 g, 0.05 equiv, 3.5 mmol), K₂CO₃ (14.41 g, 1.5 equiv, 104.3 mmol), N,N-dimethylacetamide (DMA, 278 mL) and PPh₃ (1.82 g, 0.1 equiv, 7.0 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-51 (14.34 g, yield 74%).

Through FAB-MS measurement, mass number m/z=278 was observed as an ion peak, and Intermediate IM-51 was identified.

### Synthesis of Compound N4

Under an Ar atmosphere, to a 300 mL, three-neck flask, IM-51 (8.00 g, 28.7 mmol), Pd(dba)₂ (0.50 g, 0.03 equiv, 0.9 mmol), NaOtBu (5.52 g, 2.0 equiv, 57.4 mmol), toluene (144 mL), bis[4-(naphthalene-2-yl)phenyl]amine (13.31 g, 1.1 equiv, 31.6 mmol) and tBu₃P (0.58 g, 0.1 equiv, 2.9 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound N4 of a solid (16.38 g, yield 86%).

Through FAB-MS measurement, mass number m/z=663 was observed as an ion peak, and Compound N4 was identified.

### 26. Synthesis of Compound N36

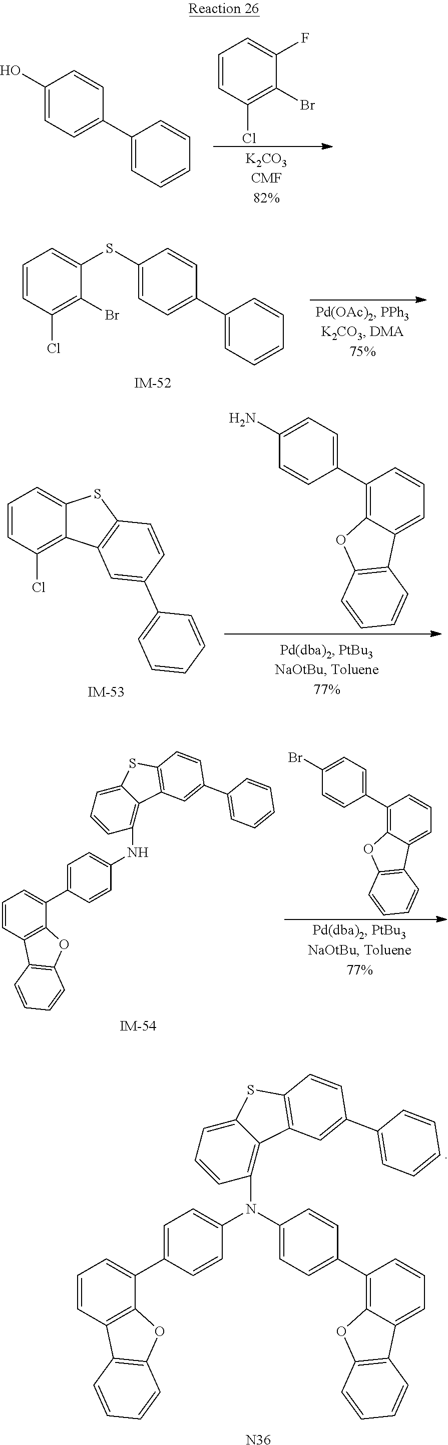

### Synthesis of Intermediate IM-52

Under an Ar atmosphere, to a 1000 mL, three-neck flask, [1,1′-biphenyl]-4-thiol (20.00 g, 107.4 mmol), 2-bromo-1-chloro-3-fluorobenzene (44.98 g, 2 equiv, 214.7 mmol), DMF (537 mL) and K₂CO₃ (59.36 g, 4 equiv, 429.5 mmol) were added in order, and heated to about 140° C. and stirred. After cooling to room temperature, H₂O was added to the reaction solution, and an organic layer was extracted with toluene, washed with a saturated saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-52 (33.08 g, yield 82%).

Through FAB-MS measurement, mass number m/z=375 was observed as an ion peak, and Intermediate IM-52 was identified.

### Synthesis of Intermediate IM-53

Under an Ar atmosphere, to a 500 mL, three-neck flask, IM-52 (25.00 g, 66.5 mmol), Pd(OAc)₂ (0.74 g, 0.05 equiv, 3.3 mmol), K₂CO₃ (13.79 g, 1.5 equiv, 99.8 mmol), N,N-dimethylacetamide (DMA, 266 mL) and PPh₃ (1.74 g, 0.1 equiv, 6.7 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-53 (14.71 g, yield 75%).

Through FAB-MS measurement, mass number m/z=294 was observed as an ion peak, and Intermediate IM-53 was identified.

### Synthesis of Intermediate IM-54

Under an Ar atmosphere, to a 500 mL, three-neck flask, IM-53 (10.00 g, 33.9 mmol), Pd(dba)₂ (0.58 g, 0.03 equiv, 1.0 mmol), NaOtBu (3.26 g, 1.0 equiv, 33.9 mmol), toluene (170 mL), 4-(dibenzofuran-4-yl)aniline (9.68 g, 1.1 equiv, 37.3 mmol) and tBu₃P (0.69 g, 0.1 equiv, 3.4 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-54 (13.52 g, yield 77%).

Through FAB-MS measurement, mass number m/z=517 was observed as an ion peak, and Intermediate IM-54 was identified.

### Synthesis of Compound N36

Under an Ar atmosphere, to a 300 mL, three-neck flask, IM-54 (8.00 g, 15.5 mmol), Pd(dba)₂ (0.27 g, 0.03 equiv, 0.5 mmol), NaOtBu (2.97 g, 2.0 equiv, 30.9 mmol), toluene (78 mL), 4-(4-bromophenyl)dibenzofuran (5.49 g, 1.1 equiv, 17.0 mmol) and tBu₃P (0.31 g, 0.1 equiv, 1.5 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound N36 of a solid (9.51 g, yield 81%).

Through FAB-MS measurement, mass number m/z=759 was observed as an ion peak, and Compound N36 was identified.

### 27. Synthesis of Compound A106

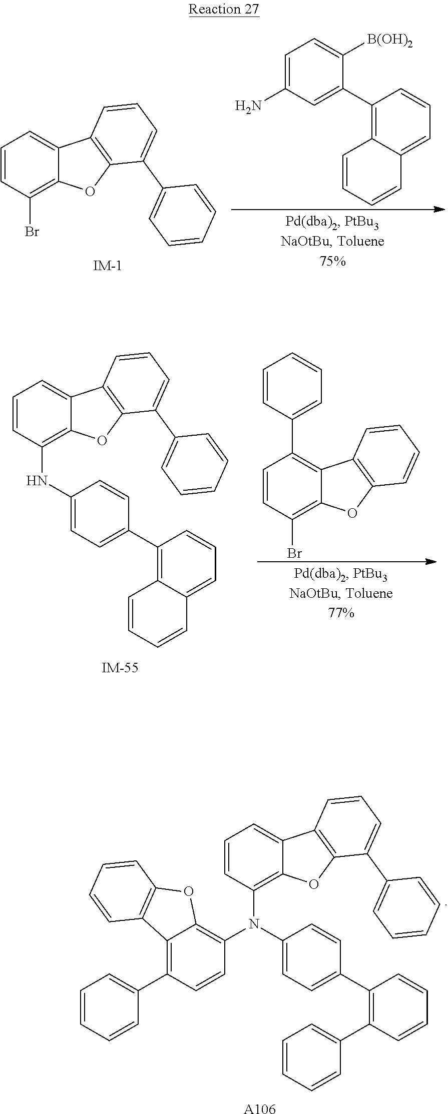

### Synthesis of Intermediate IM-55

Under an Ar atmosphere, to a 500 mL, three-neck flask, IM-1 (15.00 g, 46.4 mmol), Pd(dba)₂ (0.80 g, 0.03 equiv, 1.4 mmol), NaOtBu (4.46 g, 1.0 equiv, 46.4 mmol), toluene (232 mL), 4-(naphthalen-1-yl)aniline (11.20 g, 1.1 equiv, 51.1 mmol) and tBu₃P (0.94 g, 0.1 equiv, 4.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-55 (16.07 g, yield 75%).

Through FAB-MS measurement, mass number m/z=461 was observed as an ion peak, and Intermediate IM-55 was identified.

### Synthesis of Compound A106

Under an Ar atmosphere, to a 300 mL, three-neck flask, IM-55 (10.00 g, 21.7 mmol), Pd(dba)₂ (0.37 g, 0.03 equiv, 0.6 mmol), NaOtBu (4.16 g, 2.0 equiv, 43.3 mmol), toluene (108 mL), 4-bromo-1-phenyldibenzofuran (7.70 g, 1.1 equiv, 23.8 mmol) and tBu₃P (0.44 g, 0.1 equiv, 2.2 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound A106 of a solid (11.74 g, yield 77%).

Through FAB-MS measurement, mass number m/z=703 was observed as an ion peak, and Compound A106 was identified.

### 28. Synthesis of Compound A107

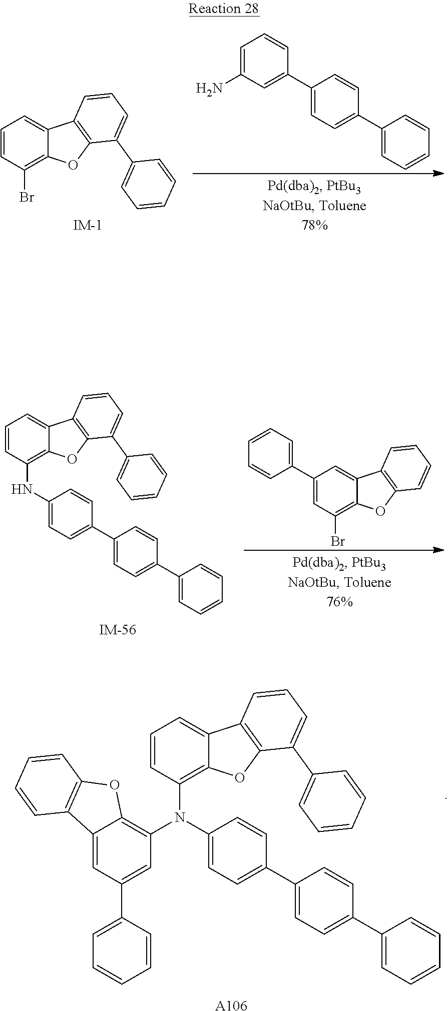

### Synthesis of Intermediate IM-56

Under an Ar atmosphere, to a 500 mL, three-neck flask, IM-1 (15.00 g, 46.4 mmol), Pd(dba)₂ (0.80 g, 0.03 equiv, 1.4 mmol), NaOtBu (4.46 g, 1.0 equiv, 46.4 mmol), toluene (232 mL), [1,1′:4′,1″-terphenyl]-4-amine (12.52 g, 1.1 equiv, 51.1 mmol) and tBu₃P (0.94 g, 0.1 equiv, 4.6 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-56 (17.65 g, yield 78%).

Through FAB-MS measurement, mass number m/z=487 was observed as an ion peak, and Intermediate IM-56 was identified.

### Synthesis of Compound A107

Under an Ar atmosphere, to a 300 mL, three-neck flask, IM-56 (10.00 g, 20.5 mmol), Pd(dba)₂ (0.35 g, 0.03 equiv, 0.6 mmol), NaOtBu (3.94 g, 2.0 equiv, 41.0 mmol), toluene (108 mL), 4-bromo-2-phenyldibenzofuran (7.29 g, 1.1 equiv, 22.6 mmol) and tBu₃P (0.41 g, 0.1 equiv, 2.1 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound A107 of a solid (11.38 g, yield 76%).

Through FAB-MS measurement, mass number m/z=729 was observed as an ion peak, and Compound A107 was identified.

### 29. Synthesis of Compound A17

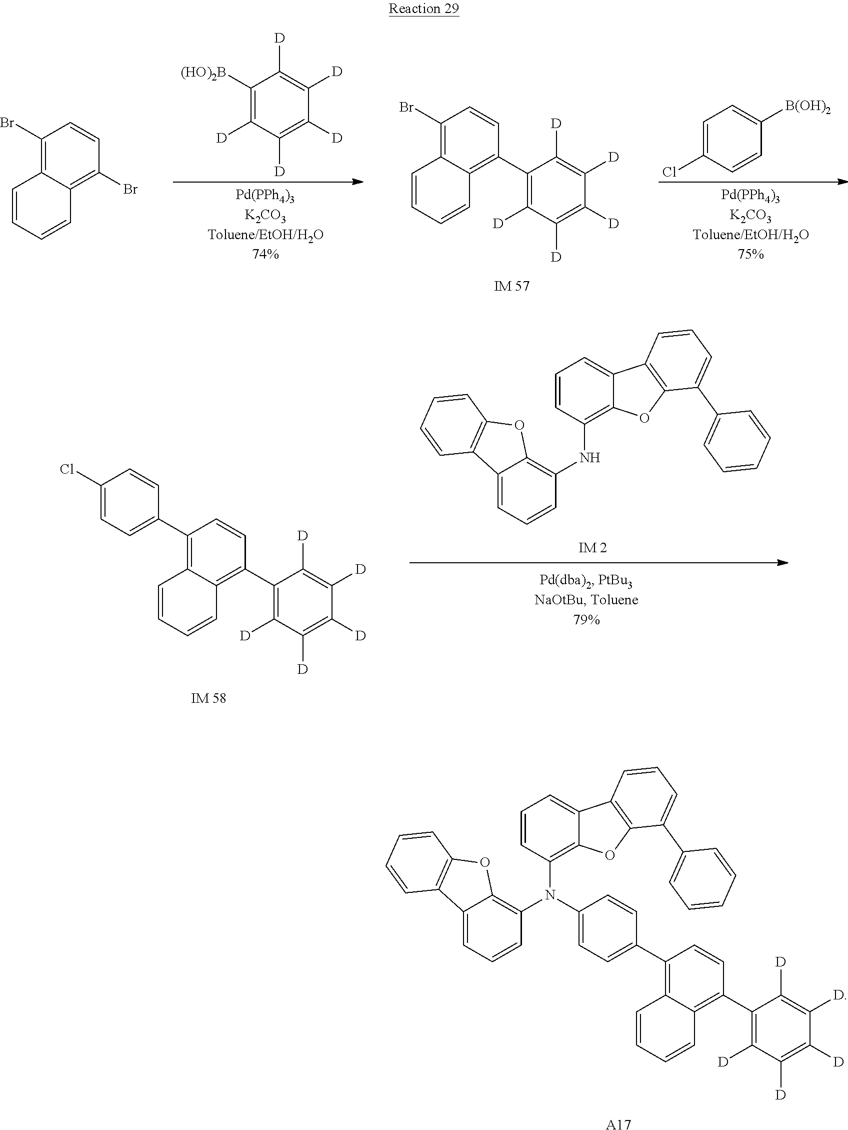

### Synthesis of Intermediate IM-57

Under an Ar atmosphere, to a 1000 mL, three-neck flask, 1,4-dibromonaphthalene (25.00 g, 87.4 mmol), (phenyl-d₅)boronic acid (12.21 g, 1.1 equiv, 96.2 mmol), K₂CO₃ (36.25 g, 3.0 equiv, 262.3 mmol), Pd(PPh₃)₄ (5.05 g, 0.05 eq, 4.4 mmol) and 612 mL of a mixture solution of toluene/EtOH/H₂O (4/2/1) were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-57 (18.64 g, yield 74%).

Through FAB-MS measurement, mass number m/z=288 was observed as an ion peak, and Intermediate IM-57 was identified.

### Synthesis of Intermediate IM-58

Under an Ar atmosphere, to a 1000 mL, three-neck flask, IM-57 (15.00 g, 52.0 mmol), 4-chlorophenylboronic acid (8.95 g, 1.1 equiv, 57.3 mmol), K₂CO₃ (21.58 g, 3.0 equiv, 156.1 mmol), Pd(PPh₃)₄ (3.00 g, 0.05 eq, 2.6 mmol) and 364 mL of a mixture solution of toluene/EtOH/H₂O (4/2/1) were added in order were added in order, and heated to about 80° C. and stirred. After cooling to room temperature, the reaction solution was extracted with toluene. An aqueous layer was removed, and an organic layer was washed with a saturated saline solution and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-58 (12.49 g, yield 75%).

Through FAB-MS measurement, mass number m/z=319 was observed as an ion peak, and Intermediate IM-58 was identified.

### Synthesis of Compound A17

Under an Ar atmosphere, to a 300 mL, three-neck flask, IM-2 (10.00 g, 23.5 mmol), Pd(dba)₂ (0.41 g, 0.03 equiv, 0.7 mmol), NaOtBu (4.52 g, 2.0 equiv, 47.0 mmol), toluene (118 mL), IM-58 (8.27 g, 1.1 equiv, 25.9 mmol) and tBu₃P (0.48 g, 0.1 equiv, 2.4 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound A17 of a solid (13.16 g, yield 79%).

Through FAB-MS measurement, mass number m/z=708 was observed as an ion peak, and Compound A17 was identified.

### 30. Synthesis of Compound J19

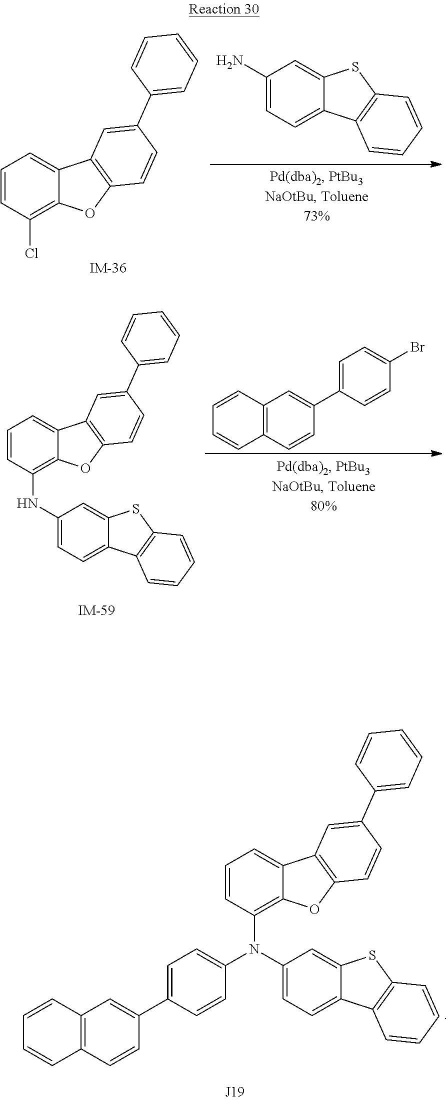

### Synthesis of Intermediate IM-59

Under an Ar atmosphere, to a 500 mL, three-neck flask, IM-36 (15.00 g, 53.8 mmol), Pd(dba)₂ (0.93 g, 0.03 equiv, 1.6 mmol), NaOtBu (5.17 g, 1.0 equiv, 53.8 mmol), toluene (269 mL), 3-aminodibenzothiophene (11.80 g, 1.1 equiv, 59.2 mmol) and tBu₃P (1.09 g, 0.1 equiv, 5.4 mmol) were added in order, and heated, refluxed and stirred. After cooling to room temperature, water was added to the reaction solution, and an organic layer was separately taken. Toluene was added to an aqueous layer, and an organic layer was extracted further. Organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-59 (17.35 g, yield 73%).

Through FAB-MS measurement, mass number m/z=441 was observed as an ion peak, and Intermediate IM-59 was identified.

### Synthesis of Compound J19

Under an Ar atmosphere, to a 300 mL, three-neck flask, IM-59 (10.00 g, 22.6 mmol), Pd(dba)₂ (0.39 g, 0.03 equiv, 0.7 mmol), NaOtBu (4.35 g, 2.0 equiv, 45.3 mmol), toluene (113 mL), 2-(4-bromophenyl)naphthalene (7.05 g, 1.1 equiv, 24.9 mmol) and tBu₃P (0.46 g, 0.1 equiv, 2.3 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound J19 of a solid (11.66 g, yield 80%).

Through FAB-MS measurement, mass number m/z=643 was observed as an ion peak, and Compound J19 was identified.

### 31. Synthesis of Compound K11

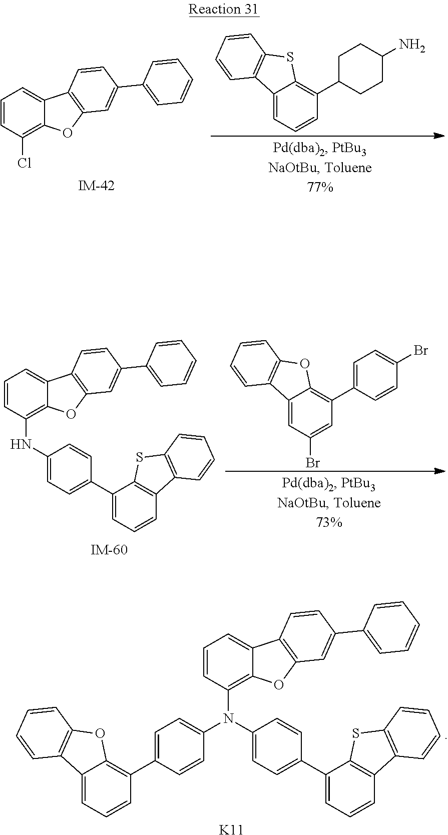

### Synthesis of Intermediate IM-60

Under an Ar atmosphere, to a 500 mL, three-neck flask, IM-42 (15.00 g, 53.8 mmol), Pd(dba)₂ (0.93 g, 0.03 equiv, 1.6 mmol), NaOtBu (5.17 g, 1.0 equiv, 53.8 mmol), toluene (269 mL), 4-(dibenzothiophen-4-yl)aniline (16.30 g, 1.1 equiv, 59.2 mmol) and tBu₃P (1.09 g, 0.1 equiv, 5.4 mmol) were added in order, and heated, refluxed and stirred. After cooling to room temperature, water was added to the reaction solution, and an organic layer was separately taken. Toluene was added to an aqueous layer, and an organic layer was extracted further. Organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Intermediate IM-60 (21.45 g, yield 77%).

Through FAB-MS measurement, mass number m/z=517 was observed as an ion peak, and Intermediate IM-60 was identified.

### Synthesis of Compound K11

Under an Ar atmosphere, to a 300 mL, three-neck flask, IM-60 (10.00 g, 19.3 mmol), Pd(dba)₂ (0.33 g, 0.03 equiv, 0.6 mmol), NaOtBu (3.71 g, 2.0 equiv, 38.6 mmol), toluene (97 mL), 4-(4-bromophenyl)dibenzofuran (6.87 g, 1.1 equiv, 21.2 mmol) and tBu₃P (0.39 g, 0.1 equiv, 1.9 mmol) were added in order, and heated, refluxed and stirred. After cooling to room temperature, water was added to the reaction solution, and an organic layer was separately taken. Toluene was added to an aqueous layer, and an organic layer was extracted further. The organic layers were collected, washed with a saline solution, and dried with MgSO₄. MgSO₄ was filtered, and an organic layer was concentrated. The crude product thus obtained was separated by silica gel column chromatography (utilizing a mixture solvent of hexane and toluene as a developing layer) to obtain Compound K11 of a solid (10.72 g, yield 73%).

Through FAB-MS measurement, mass number m/z=759 was observed as an ion peak, and Compound K11 was identified.

### Examples of Manufacturing Devices

Luminescence devices were manufactured utilizing Example Compounds and Comparative Compounds as materials of a hole transport region.

### Example Compounds

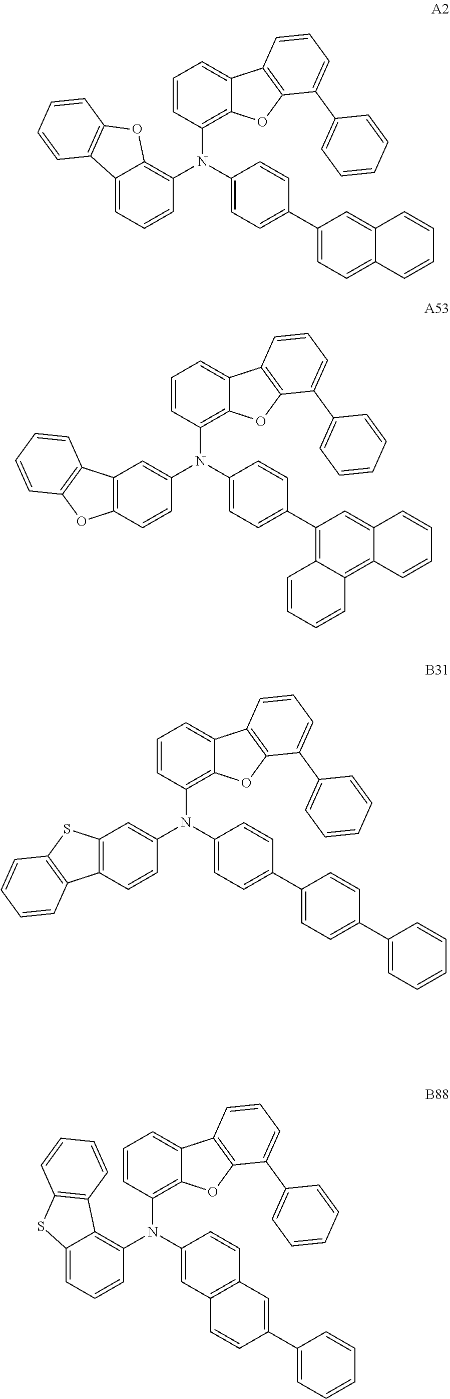 [SMILES: c1ccc(-c2cccc3c2oc2c(N(c4ccc(-c5ccc6ccccc6c5)cc4)c4cccc5c4oc4ccccc45)cccc23)cc1 ; c1ccc(-c2cccc3c2oc2c(N(c4ccc(-c5cc6ccccc6c6ccccc56)cc4)c4ccc5oc6ccccc6c5c4)cccc23)cc1 ; c1ccc(-c2ccc(-c3ccc(N(c4ccc5c(c4)sc4ccccc45)c4cccc5c4oc4c(-c6ccccc6)cccc45)cc3)cc2)cc1 ; c1ccc(-c2ccc3cc(N(c4cccc5c4oc4c(-c6ccccc6)cccc45)c4cccc5sc6ccccc6c45)ccc3c2)cc1]

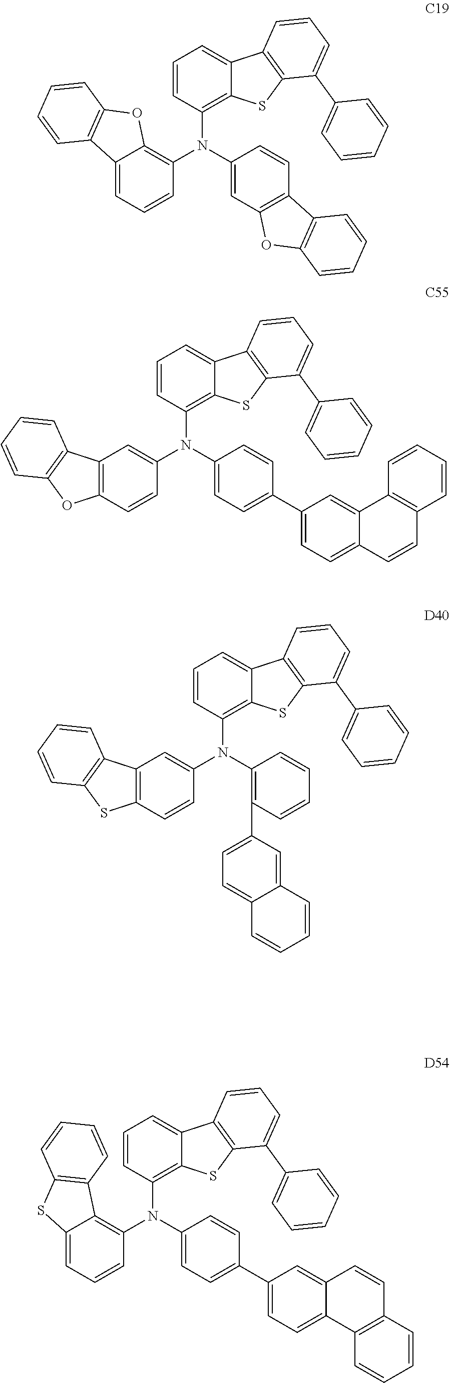 [SMILES: c1ccc(-c2cccc3c2sc2c(N(c4ccc5c(c4)oc4ccccc45)c4cccc5c4oc4ccccc45)cccc23)cc1 ; c1ccc(-c2cccc3c2sc2c(N(c4ccc(-c5ccc6ccc7ccccc7c6c5)cc4)c4ccc5oc6ccccc6c5c4)cccc23)cc1 ; c1ccc(-c2cccc3c2sc2c(N(c4ccc5sc6ccccc6c5c4)c4ccccc4-c4ccc5ccccc5c4)cccc23)cc1 ; c1ccc(-c2cccc3c2sc2c(N(c4ccc(-c5ccc6c(ccc7ccccc76)c5)cc4)c4cccc5sc6ccccc6c45)cccc23)cc1]

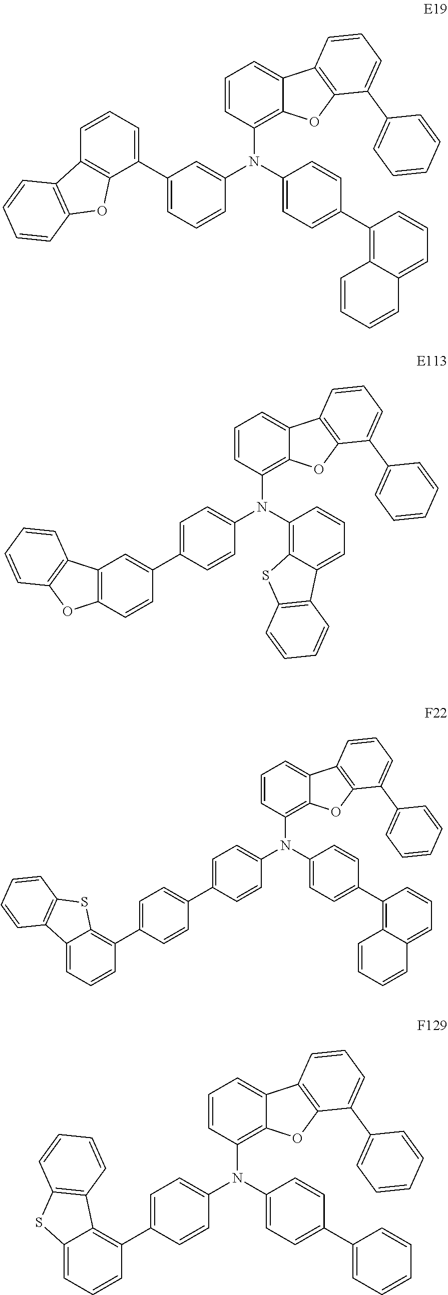 [SMILES: c1ccc(-c2cccc3c2oc2c(N(c4ccc(-c5cccc6ccccc56)cc4)c4cccc(-c5cccc6c5oc5ccccc56)c4)cccc23)cc1 ; c1ccc(-c2cccc3c2oc2c(N(c4ccc(-c5ccc6oc7ccccc7c6c5)cc4)c4cccc5c4sc4ccccc45)cccc23)cc1 ; c1ccc(-c2cccc3c2oc2c(N(c4ccc(-c5ccc(-c6cccc7c6sc6ccccc67)cc5)cc4)c4ccc(-c5cccc6ccccc56)cc4)cccc23)cc1 ; c1ccc(-c2ccc(N(c3ccc(-c4cccc5sc6ccccc6c45)cc3)c3cccc4c3oc3c(-c5ccccc5)cccc34)cc2)cc1]

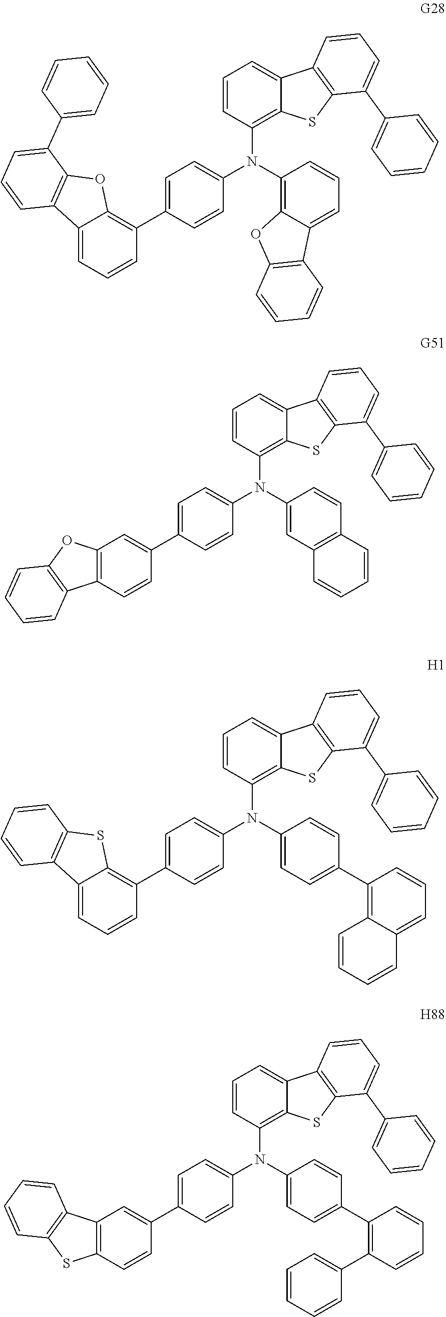 [SMILES: c1ccc(-c2cccc3c2oc2c(-c4ccc(N(c5cccc6c5oc5ccccc56)c5cccc6c5sc5c(-c7ccccc7)cccc56)cc4)cccc23)cc1 ; c1ccc(-c2cccc3c2sc2c(N(c4ccc(-c5ccc6c(c5)oc5ccccc56)cc4)c4ccc5ccccc5c4)cccc23)cc1 ; c1ccc(-c2cccc3c2sc2c(N(c4ccc(-c5cccc6ccccc56)cc4)c4ccc(-c5cccc6c5sc5ccccc56)cc4)cccc23)cc1 ; c1ccc(-c2ccccc2-c2ccc(N(c3ccc(-c4ccc5sc6ccccc6c5c4)cc3)c3cccc4c3sc3c(-c5ccccc5)cccc34)cc2)cc1]

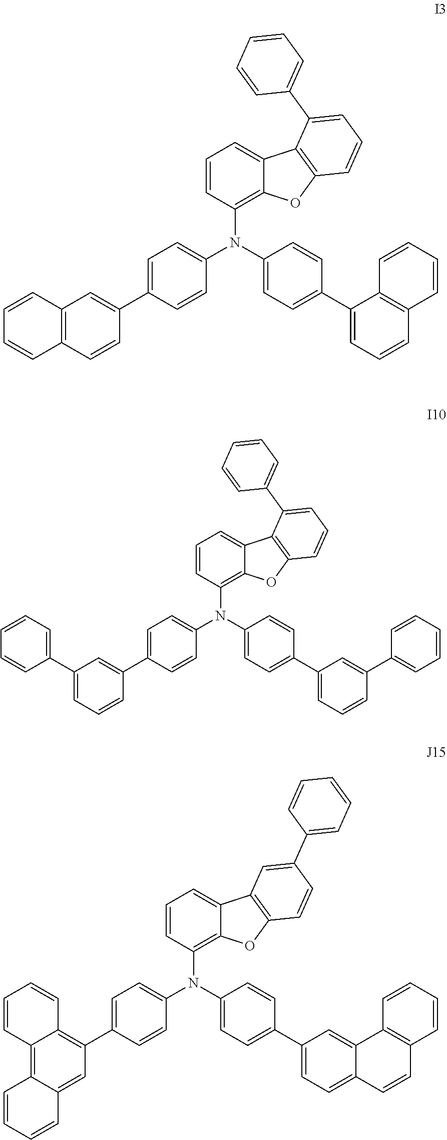 [SMILES: c1ccc(-c2cccc3oc4c(N(c5ccc(-c6ccc7ccccc7c6)cc5)c5ccc(-c6cccc7ccccc67)cc5)cccc4c23)cc1 ; c1ccc(-c2cccc(-c3ccc(N(c4ccc(-c5cccc(-c6ccccc6)c5)cc4)c4cccc5c4oc4cccc(-c6ccccc6)c45)cc3)c2)cc1 ; c1ccc(-c2ccc3oc4c(N(c5ccc(-c6ccc7ccc8ccccc8c7c6)cc5)c5ccc(-c6cc7ccccc7c7ccccc67)cc5)cccc4c3c2)cc1]

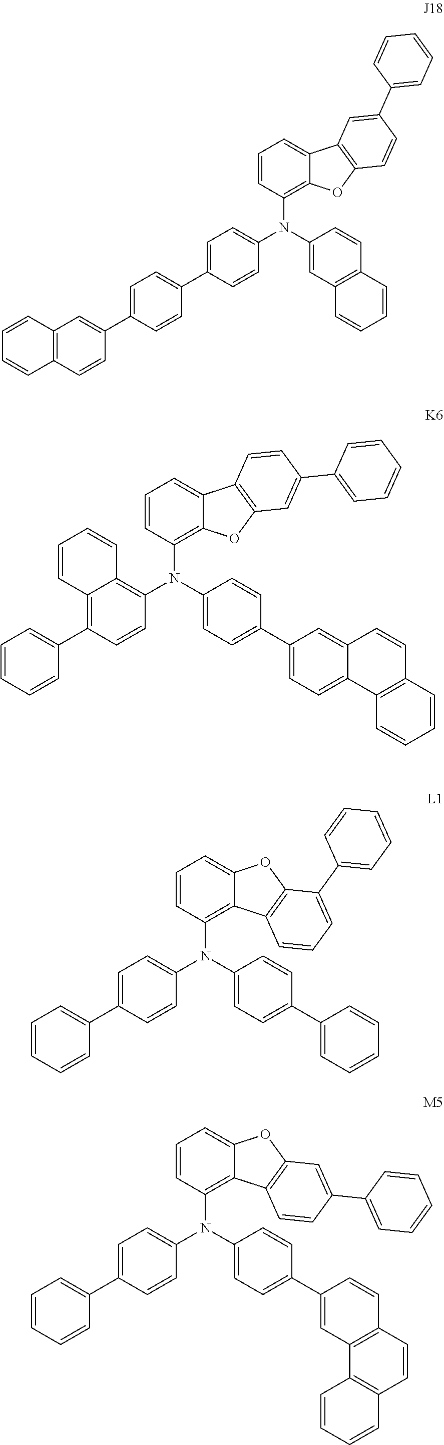 [SMILES: c1ccc(-c2ccc3oc4c(N(c5ccc(-c6ccc(-c7ccc8ccccc8c7)cc6)cc5)c5ccc6ccccc6c5)cccc4c3c2)cc1 ; c1ccc(-c2ccc3c(c2)oc2c(N(c4ccc(-c5ccc6c(ccc7ccccc76)c5)cc4)c4ccc(-c5ccccc5)c5ccccc45)cccc23)cc1 ; c1ccc(-c2ccc(N(c3ccc(-c4ccccc4)cc3)c3cccc4oc5c(-c6ccccc6)cccc5c34)cc2)cc1 ; c1ccc(-c2ccc(N(c3ccc(-c4ccc5ccc6ccccc6c5c4)cc3)c3cccc4oc5cc(-c6ccccc6)ccc5c34)cc2)cc1]

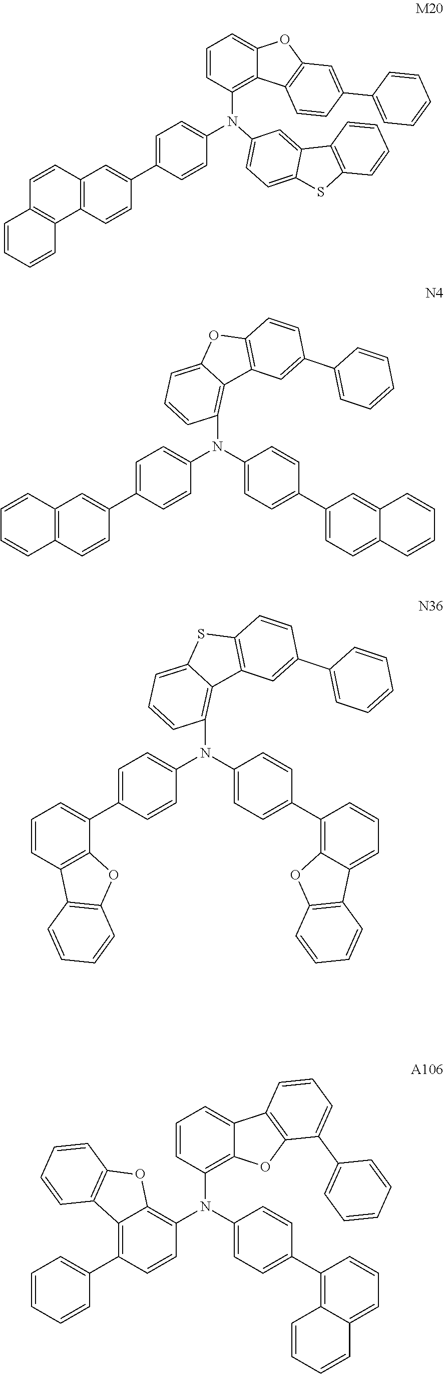 [SMILES: c1ccc(-c2ccc3c(c2)oc2cccc(N(c4ccc(-c5ccc6c(ccc7ccccc76)c5)cc4)c4ccc5sc6ccccc6c5c4)c23)cc1 ; c1ccc(-c2ccc3oc4cccc(N(c5ccc(-c6ccc7ccccc7c6)cc5)c5ccc(-c6ccc7ccccc7c6)cc5)c4c3c2)cc1 ; c1ccc(-c2ccc3sc4cccc(N(c5ccc(-c6cccc7c6oc6ccccc67)cc5)c5ccc(-c6cccc7c6oc6ccccc67)cc5)c4c3c2)cc1 ; c1ccc(-c2cccc3c2oc2c(N(c4ccc(-c5cccc6ccccc56)cc4)c4ccc(-c5ccccc5)c5c4oc4ccccc45)cccc23)cc1]

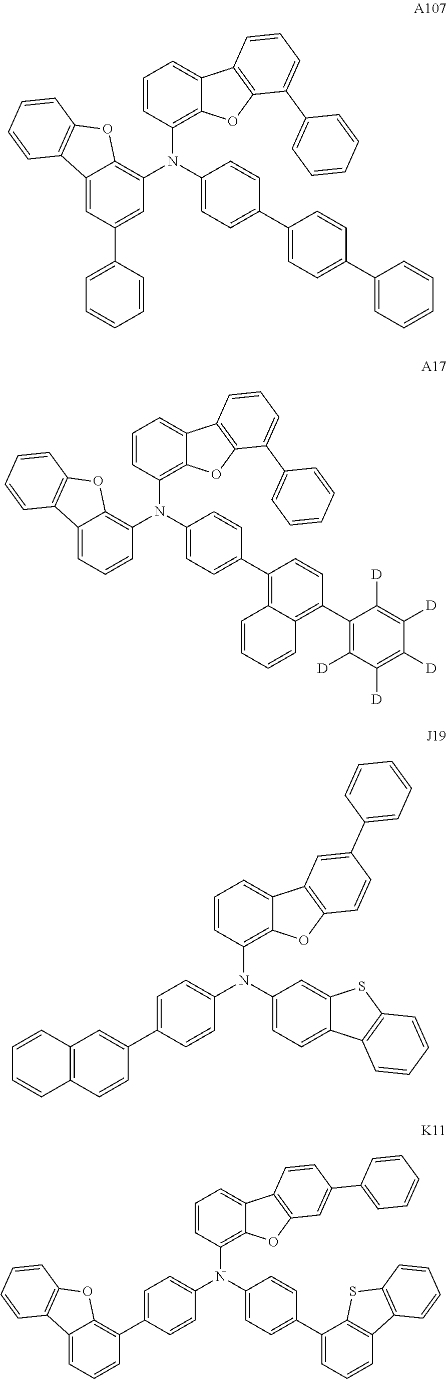 [SMILES: c1ccc(-c2ccc(-c3ccc(N(c4cc(-c5ccccc5)cc5c4oc4ccccc45)c4cccc5c4oc4c(-c6ccccc6)cccc45)cc3)cc2)cc1 ; [2H]c1c([2H])c([2H])c(-c2ccc(-c3ccc(N(c4cccc5c4oc4ccccc45)c4cccc5c4oc4c(-c6ccccc6)cccc45)cc3)c3ccccc23)c([2H])c1[2H] ; c1ccc(-c2ccc3oc4c(N(c5ccc(-c6ccc7ccccc7c6)cc5)c5ccc6c(c5)sc5ccccc56)cccc4c3c2)cc1 ; c1ccc(-c2ccc3c(c2)oc2c(N(c4ccc(-c5cccc6c5oc5ccccc56)cc4)c4ccc(-c5cccc6c5sc5ccccc56)cc4)cccc23)cc1]

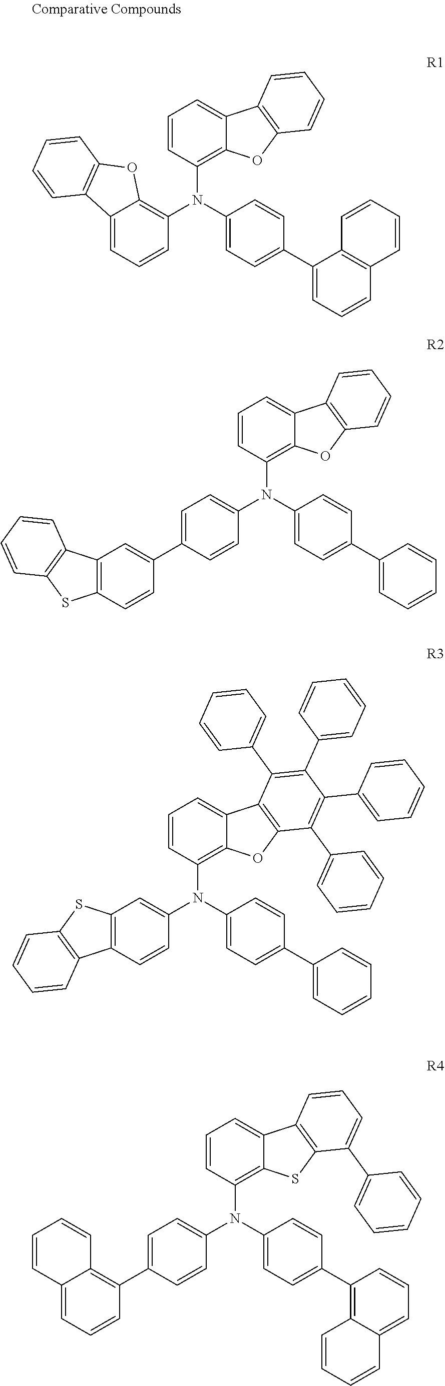 [SMILES: c1ccc2c(-c3ccc(N(c4cccc5c4oc4ccccc45)c4cccc5c4oc4ccccc45)cc3)cccc2c1 ; c1ccc(-c2ccc(N(c3ccc(-c4ccc5sc6ccccc6c5c4)cc3)c3cccc4c3oc3ccccc34)cc2)cc1 ; c1ccc(-c2ccc(N(c3ccc4c(c3)sc3ccccc34)c3cccc4c3oc3c(-c5ccccc5)c(-c5ccccc5)c(-c5ccccc5)c(-c5ccccc5)c34)cc2)cc1 ; c1ccc(-c2cccc3c2sc2c(N(c4ccc(-c5cccc6ccccc56)cc4)c4ccc(-c5cccc6ccccc56)cc4)cccc23)cc1]

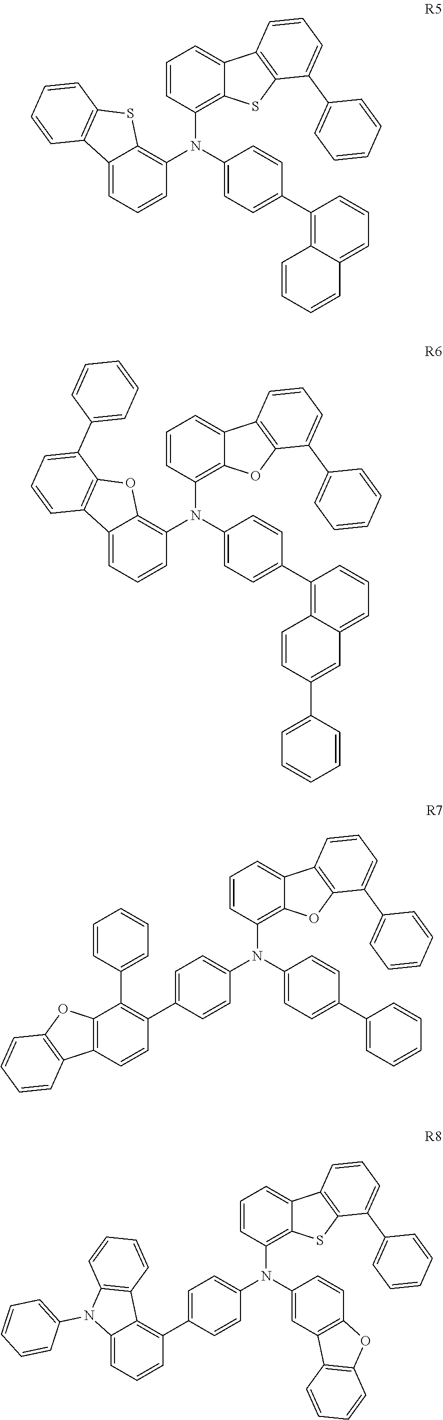 [SMILES: c1ccc(-c2cccc3c2sc2c(N(c4ccc(-c5cccc6ccccc56)cc4)c4cccc5c4sc4ccccc45)cccc23)cc1 ; c1ccc(-c2ccc3c(-c4ccc(N(c5cccc6c5oc5c(-c7ccccc7)cccc56)c5cccc6c5oc5c(-c7ccccc7)cccc56)cc4)cccc3c2)cc1 ; c1ccc(-c2ccc(N(c3ccc(-c4ccc5c(oc6ccccc65)c4-c4ccccc4)cc3)c3cccc4c3oc3c(-c5ccccc5)cccc34)cc2)cc1 ; c1ccc(-c2cccc3c2sc2c(N(c4ccc(-c5cccc6c5c5ccccc5n6-c5ccccc5)cc4)c4ccc5oc6ccccc6c5c4)cccc23)cc1]

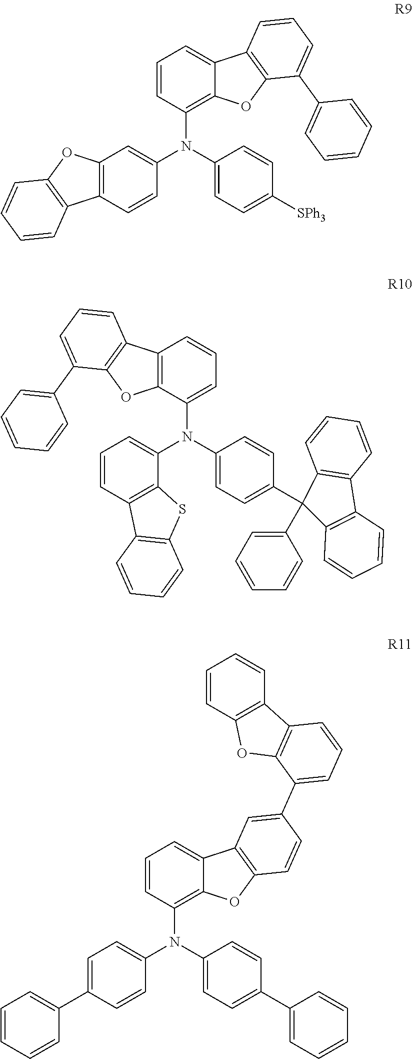

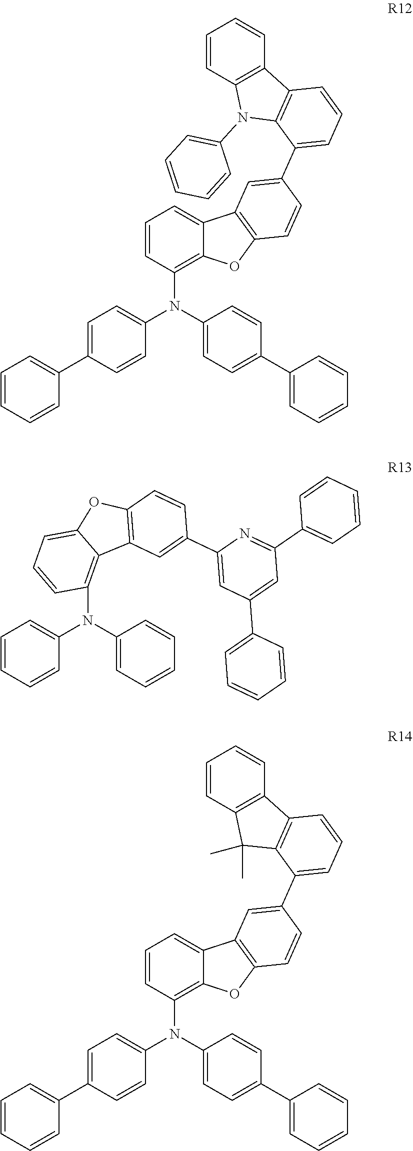 [SMILES: c1ccc(-c2ccc(N(c3ccc(-c4ccccc4)cc3)c3cccc4c3oc3ccc(-c5cccc6c7ccccc7n(-c7ccccc7)c56)cc34)cc2)cc1 ; c1ccc(-c2cc(-c3ccccc3)nc(-c3ccc4oc5cccc(N(c6ccccc6)c6ccccc6)c5c4c3)c2)cc1 ; CC1(C)c2ccccc2-c2cccc(-c3ccc4oc5c(N(c6ccc(-c7ccccc7)cc6)c6ccc(-c7ccccc7)cc6)cccc5c4c3)c21]

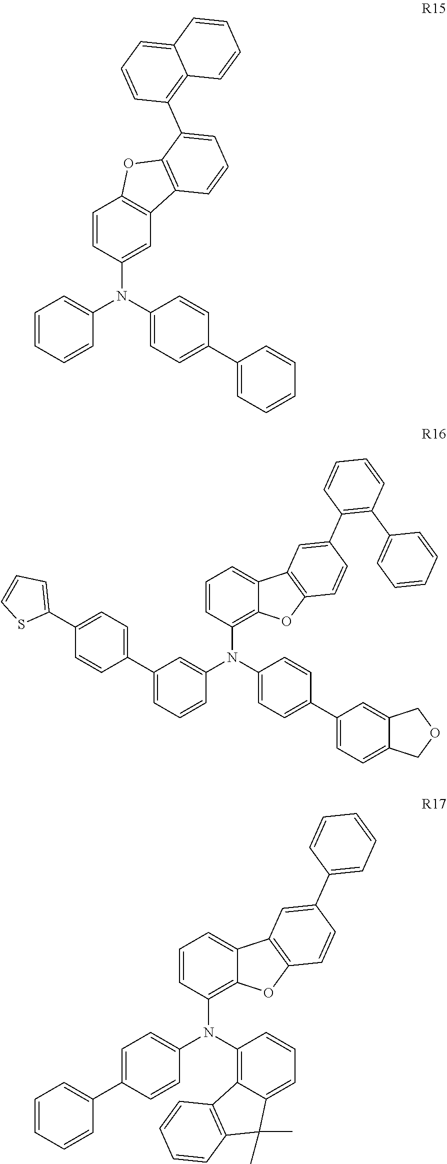 [SMILES: c1ccc(-c2ccc(N(c3ccccc3)c3ccc4oc5c(-c6cccc7ccccc67)cccc5c4c3)cc2)cc1 ; c1ccc(-c2ccccc2-c2ccc3oc4c(N(c5ccc(-c6ccc7c(c6)COC7)cc5)c5cccc(-c6ccc(-c7cccs7)cc6)c5)cccc4c3c2)cc1 ; CC1(C)c2ccccc2-c2c(N(c3ccc(-c4ccccc4)cc3)c3cccc4c3oc3ccc(-c5ccccc5)cc34)cccc21]

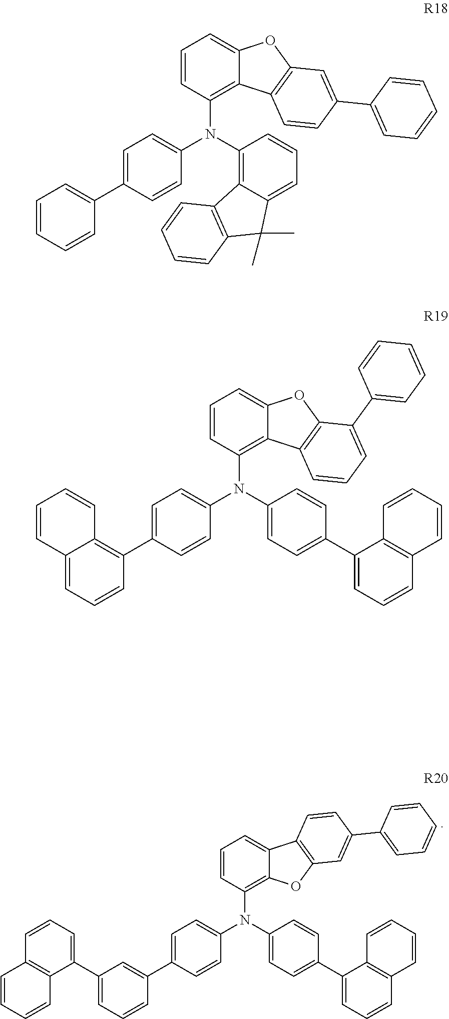 [SMILES: CC1(C)c2ccccc2-c2c(N(c3ccc(-c4ccccc4)cc3)c3cccc4oc5cc(-c6ccccc6)ccc5c34)cccc21 ; c1ccc(-c2cccc3c2oc2cccc(N(c4ccc(-c5cccc6ccccc56)cc4)c4ccc(-c5cccc6ccccc56)cc4)c23)cc1 ; c1ccc(-c2ccc3c(c2)oc2c(N(c4ccc(-c5cccc(-c6cccc7ccccc67)c5)cc4)c4ccc(-c5cccc6ccccc56)cc4)cccc23)cc1]

The luminescence devices of the Examples and Comparative Examples were manufactured by the method below. On a glass substrate, ITO with a thickness of about 150 nm was patterned, washed with ultrapure water and treated with UV ozone to form a first electrode. Then, 2-TNATA was deposited to a thickness of about 60 nm, and an Example Compound or Comparative Compound was deposited to a thickness of about 30 nm to form a hole transport layer. After that, an emission layer was formed utilizing ADN doped with 3% TBP to a thickness of about 25 nm. On the emission layer, a layer with a thickness of about 25 nm was formed utilizing Alq3, and a layer with a thickness of about 1 nm was formed utilizing LiF to form an electron transport region. Then, a second electrode with a thickness of about 100 nm was formed utilizing aluminum (Al). All layers were formed by a vacuum deposition method.

### Evaluation of Properties of Luminescence Devices

The voltage, emission efficiency and life span of each luminescence device were measured and shown in Table 1 and Table 2 below. The voltage and emission efficiency were measured at a current efficiency of 10 mA/cm², and the half-life corresponds to results at 1.0 mA/cm².

TABLE 1

| | | | Emission | |
|---|---|---|---|---|
| | | Voltage | efficiency | Life span |
| | Hole transport layer | (V) | (cd/A) | LT50 (h) |
| Example 1 | Example Compound | 5.4 | 7.3 | 1950 |
| | A2 | | | |
| Example 2 | Example Compound | 5.5 | 7.2 | 1900 |
| | A53 | | | |
| Example 3 | Example Compound | 5.4 | 7.0 | 2000 |
| | B31 | | | |
| Example 4 | Example Compound | 5.6 | 7.4 | 1950 |
| | B88 | | | |
| Example 5 | Example Compound | 5.4 | 7.2 | 2000 |
| | C19 | | | |
| Example 6 | Example Compound | 5.6 | 7.3 | 1950 |
| | C55 | | | |
| Example 7 | Example Compound | 5.5 | 7.3 | 1850 |
| | D40 | | | |
| Example 8 | Example Compound | 5.4 | 7.4 | 1900 |
| | D54 | | | |
| Example 9 | Example Compound | 5.4 | 7.2 | 2000 |
| | E19 | | | |
| Example 10 | Example Compound | 5.6 | 7.2 | 2050 |
| | E113 | | | |
| Example 11 | Example Compound | 5.5 | 7.0 | 2100 |
| | F22 | | | |
| Example 12 | Example Compound | 5.4 | 7.0 | 2050 |
| | F129 | | | |
| Example 13 | Example Compound | 5.6 | 7.1 | 2100 |
| | G28 | | | |
| Example 14 | Example Compound | 5.5 | 6.9 | 2100 |
| | G51 | | | |
| Example 15 | Example Compound | 5.5 | 7.0 | 2000 |
| | H1 | | | |
| Example 16 | Example Compound | 5.5 | 7.1 | 2050 |
| | H88 | | | |
| Example 17 | Example Compound | 5.4 | 7.4 | 1950 |
| | A106 | | | |
| Example 18 | Example Compound | 5.5 | 7.3 | 2050 |
| | A107 | | | |
| Example 19 | Example Compound | 5.6 | 7.3 | 2000 |
| | A17 | | | |
| Comparative | Comparative | 6.0 | 6.2 | 1700 |
| Example 1 | Compound R1 | | | |
| Comparative | Comparative | 6.1 | 6.1 | 1750 |
| Example 2 | Compound R2 | | | |
| Comparative | Comparative | 6.0 | 5.5 | 1450 |
| Example 3 | Compound R3 | | | |
| Comparative | Comparative | 6.1 | 5.6 | 1600 |
| Example 4 | Compound R4 | | | |
| Comparative | Comparative | 5.9 | 5.8 | 1600 |
| Example 5 | Compound R5 | | | |
| Comparative | Comparative | 5.8 | 6.2 | 1750 |
| Example 6 | Compound R6 | | | |
| Comparative | Comparative | 6.2 | 6.1 | 1650 |
| Example 7 | Compound R7 | | | |
| Comparative | Comparative | 5.9 | 5.9 | 1650 |
| Example 8 | Compound R8 | | | |
| Comparative | Comparative | 6.0 | 6.3 | 1700 |
| Example 9 | Compound R9 | | | |
| Comparative | Comparative | 6.1 | 6.0 | 1650 |
| Example 10 | Compound R10 | | | |

TABLE 2

| | | | Emission | |
|---|---|---|---|---|
| | | Voltage | efficiency | Life |
| | Hole transport layer | (V) | (cd/A) | LT50 (h) |
| Example 20 | Example Compound | 5.5 | 6.7 | 2000 |
| | I3 | | | |
| Example 21 | Example Compound | 5.6 | 6.8 | 1950 |
| | I10 | | | |
| Example 22 | Example Compound | 5.4 | 7.0 | 2000 |
| | J15 | | | |
| Example 23 | Example Compound | 5.5 | 7.1 | 2050 |
| | J18 | | | |
| Example 24 | Example Compound | 5.6 | 6.9 | 1950 |
| | K6 | | | |
| Example 25 | Example Compound | 5.6 | 7.2 | 1850 |
| | L1 | | | |
| Example 26 | Example Compound | 5.5 | 7.3 | 1900 |
| | M5 | | | |
| Example 27 | Example Compound | 5.5 | 7.4 | 1900 |
| | M20 | | | |
| Example 28 | Example Compound | 5.4 | 7.3 | 2000 |
| | N4 | | | |
| Example 29 | Example Compound | 5.5 | 7.2 | 1950 |
| | N36 | | | |
| Example 30 | Example Compound | 5.6 | 7.2 | 2200 |
| | J19 | | | |
| Example 31 | Example Compound | 5.5 | 7.4 | 2150 |
| | K11 | | | |
| Comparative | Comparative | 6.1 | 6.2 | 1600 |
| Example 11 | Compound R11 | | | |
| Comparative | Comparative | 5.9 | 6.2 | 1650 |
| Example 12 | Compound R12 | | | |
| Comparative | Comparative | 6.5 | 5.4 | 1450 |
| Example 13 | Compound R13 | | | |
| Comparative | Comparative | 6.0 | 6.0 | 1550 |
| Example 14 | Compound R14 | | | |
| Comparative | Comparative | 5.8 | 5.8 | 1600 |
| Example 15 | Compound R15 | | | |
| Comparative | Comparative | 6.2 | 5.5 | 1500 |
| Example 16 | Compound R16 | | | |
| Comparative | Comparative | 6.2 | 6.0 | 1650 |
| Example 17 | Compound R17 | | | |
| Comparative | Comparative | 6.2 | 6.3 | 1600 |
| Example 18 | Compound R18 | | | |
| Comparative | Comparative | 5.7 | 6.5 | 1650 |
| Example 19 | Compound R19 | | | |
| Comparative | Comparative | 5.8 | 5.4 | 1700 |
| Example 20 | Compound R20 | | | |

Table 1 shows the results on Examples 1 to 19 and Comparative Examples 1 to 10. Table 2 shows the results on Examples 20 to 31 and Comparative Examples 11 to 20. Referring to Table 1 and Table 2, it could be confirmed that Examples 1 to 31 accomplished low voltages, high efficiency and long life span at the same time (e.g., simultaneously) when compared with Comparative Examples 1 to 20.

The amine compound according to embodiments of the present disclosure introduces a substituent into a dibenzofuran or dibenzothiophene skeleton, and shows improved heat resistance and charge tolerance, thereby accomplishing the decrease of a voltage and/or the increase of life span and/or efficiency. In addition, it may be considered that the symmetry of a molecule was degraded (e.g., decreased), and crystallization was restrained (e.g., an amorphous solid form became favored) by the dibenzofuran or dibenzothiophene skeleton having a substituent, and accordingly, layer quality could be improved, hole transport properties could be improved, and emission efficiency could be improved.

In Compound Group 1 shown in Table 1, at least one of R₂ or R₃ is required to include a dibenzoheterole group as represented by Formula 2-3, but in Compound Groups 2 and 3 shown in Table 2, excellent or suitable device properties were shown even though R₂ and R₃ are not dibenzoheterole. In case of Compound Group 1 of Table 1, a heteroatom included in Formula 2-1 was influenced by a Rₕ substituent having a large volume and was covered in three dimensions, and accordingly, the improving effects of hole transport capacity by a heteroatom was degraded. Therefore, by substituting a dibenzoheterole group in R₂ or R₃, degraded hole transport capacity was improved. In some embodiments, in case of Compound Group 2 or 3 shown in Table 2, a heteroatom included in Formula 2-1 was not covered by a substituent, and sufficient hole transport capacity could be shown.

For example, Examples 1 to 8, 17 to 19, and 27 to 30 were materials in which all multiple dibenzoheterole groups are directly bonded to a nitrogen atom, and particularly, emission efficiency was improved. This is considered that a molecule became compact, intermolecular interaction was strengthened, and hole transport capacity was improved.

Examples 9 to 16, and 29 to 31 were materials in which one selected from among multiple dibenzoheterole groups is bonded to a nitrogen atom via a connecting group, and particularly, device life span was improved. This is estimated that a HOMO orbital was broadly extended to a terminal dibenzoheterole ring via a connecting group, and material stability as a radical or radical cation active species was improved.

Comparative Examples 1 and 2 correspond to amines not having a substituted dibenzoheterole ring, and according to the decrease of the volume of a molecule, intermolecular stacking was improved, the degradation of hole transport capacity was generated, and both device efficiency and life span were degraded (e.g., simultaneously) when compared with the Examples.

Comparative Example 3 corresponds to an amine in which four phenyl groups are substituted at the same benzene ring moiety of a dibenzoheterole ring, and decomposition of a material was generated under high temperature conditions due to steric repulsion between neighboring phenyl groups, and both device efficiency and life span were degraded (e.g., simultaneously) when compared with the Examples.

Comparative Example 4 corresponds to a material having a dibenzothiophene group having a substituent at position 6 but is an amine having only one dibenzoheterole group in a molecule, and both device efficiency and life span were degraded (e.g., simultaneously) when compared with the Examples. It is thought because that the number of dibenzoheterole group is small, the improving effects of hole transport capacity by a heteroatom were reduced, the injection of holes into an emission layer was delayed, and recombination probability in the emission layer was reduced.

Comparative Example 5 corresponds to a material having 4-dibenzothiophene groups as two dibenzoheterole groups, but both device efficiency and life span were degraded (e.g., simultaneously) when compared with the Examples. When two dibenzothiophene groups are each directly bonded to a nitrogen atom at position 4, the d orbitals of two sulfur atoms in the same molecule may approach and make interaction (e.g., participate in a through-space interaction). Accordingly, intermolecular interaction via heteroatoms between molecules may be reduced, and as a result, hole transport capacity may be reduced.

In some embodiments, as in Example 15, when two dibenzothiophene groups are bonded at each position 4, and when one of them is bonded to a nitrogen atom via a connecting group, and when the bonding positions of two dibenzothiophene groups are different as in Examples 7 and 8, the interaction between two sulfur atoms in a molecule may be relieved, intermolecular interaction between intermolecular heteroatoms may act, and excellent or suitable device characteristics may be shown. In some embodiments, as in Examples 1, 5, 10 and 13, when two dibenzoheterole groups are directly bonded to a nitrogen atom at each position 4, when at least one of the heteroatoms is an oxygen atom, intramolecular interaction between the oxygen atom and the sulfur atom and between the oxygen atom and the oxygen atom is not generated, because the oxygen atom has no d orbital (e.g., the d orbital is not occupied), and excellent or suitable device characteristics may be shown.

Comparative Example 6 is a material having two dibenzoheterole skeletons having a substituent at position 6, but the symmetry of a molecule was improved (e.g., increased), and layer quality was deteriorated due to the increase of crystallinity and decrease of amorphous properties. In addition, because the surroundings of the nitrogen atom are sterically crowded, the material stability under high temperature conditions was deteriorated, and both device efficiency and life spam were degraded (e.g., simultaneously) when compared with the Examples.

In Example 13, though a material has two dibenzoheterole skeletons having a substituent at position 6, when one is bonded to a nitrogen atom via a connecting (e.g., linking) group, the symmetry of a molecule was collapsed, the steric crowd around the nitrogen atom was relieved, the material stability was improved, and excellent or suitable device characteristics were shown.

Comparative Example 7 is a material in which a phenyl group is substituted at a dibenzofuran benzene ring moiety adjacent to where an amine group is bonded. Steric electronic repulsion was generated between the substituents at positions 3 and 4 of the dibenzofuran ring, the surroundings of the nitrogen atom were crowded, material stability under high temperature conditions was degraded, and both device efficiency and life span were degraded (e.g., simultaneously) when compared with the Examples.

Comparative Example 8 is a material having a carbazole group in a molecule, but carrier balance was collapsed, and both device efficiency and life span were degraded (e.g., simultaneously) when compared with the Examples.

Comparative Examples 9 and 10 are materials having a silyl group and a fluorene group in a molecule, but both device efficiency and life span were degraded (e.g., simultaneously) when compared with the Examples. These results are thought to be obtained because a C—Si bond and a sp³ hybrid carbon atom moiety included in the fluorene skeleton were unstable under high temperature conditions, and decomposition was generated during deposition.

Comparative Examples 11 to 13 correspond to amines in which a heterocycle is bonded to (substituted on) a dibenzoheterole ring, but carrier balance was collapsed, and both device efficiency and life span were degraded (e.g., simultaneously) when compared with the Examples. Comparative Examples 14, 17 and 18 correspond to amines having a fluorene structure in a molecule, but both device efficiency and life span were degraded (e.g., simultaneously) when compared with the Examples. These results were thought to be obtained because a sp³ hybrid carbon atom moiety included in a fluorene skeleton was unstable, and decomposition was generated during deposition.

Comparative Example 15 corresponds to an amine in which a nitrogen atom is bonded at position 2 of a substituted dibenzoheterole skeleton, and through the improvement of the planarity of a molecule, crystallinity was increased, layer degradation and hole transport capacity degradation were generated, and particularly, device efficiency was degraded when compared with the Examples.

Comparative Example 16 corresponds to an amine having a thiophene ring in a molecule, but because the electron tolerance of the thiophene ring was low, deterioration of a material was generated during driving, and both device efficiency and life span were degraded (e.g., simultaneously) when compared with the Examples.

Comparative Examples 19 and 10 correspond to amines having two 1-naphthyl groups at the terminal, but both device efficiency and life span were degraded (e.g., simultaneously) when compared with the Examples. It is thought that intermolecular interaction was increased due to the influence of two 1-naphthyl groups in a molecule, and the deposition temperature of a material was increased, and layer forming properties were degraded.

The amine compound according to embodiments of the present disclosure is utilized in a hole transport region, and contributes to the decrease of the driving voltage and the increase of the efficiency and the life span of a luminescence device.

The luminescence device according to embodiments of the present disclosure has excellent or suitable efficiency.

The amine compound according to embodiments of the present disclosure may be utilized as a material of a hole transport region of a luminescence device, and the efficiency of the luminescence device may be improved.

As used herein, the terms “substantially,” “about,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

Although embodiments of the present disclosure have been described, it is understood that the present disclosure should not be limited to these embodiments, but various suitable changes and modifications can be made by one ordinary skilled in the art within the spirit and scope of the present disclosure as set forth in the following claims and equivalents thereof.

What is claimed is:

1. A luminescence device, comprising:
a first electrode;
a hole transport region on the first electrode;
an emission layer on the hole transport region;
an electron transport region on the emission layer; and
a second electrode on the electron transport region,
wherein the hole transport region comprises an amine compound represented by Formula 1: 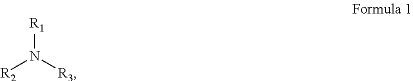
wherein in Formula 1,
R₁ is represented by Formula 2-1, and
R₂ and R₃ are each independently represented by Formula 2-2 or Formula 2-3: 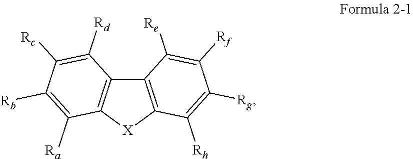
wherein in Formula 2-1,
X is O or S,
Rₐ to R_{d} are each independently a hydrogen atom, a deuterium atom, or a halogen atom, or combined with an adjacent group to form a ring, where any one selected from among Rₐ to R_{d} is a site bonded to nitrogen of Formula 1,
any one selected from among Rₑ to Rₕ is a substituted or unsubstituted alkyl group of 1 to 20 carbon atoms, or a substituted or unsubstituted aryl group of 6 to 30 ring-forming carbon atoms, and the remainder are each independently a hydrogen atom, a deuterium atom, or a halogen atom, or combined with an adjacent group to form a ring, where a fluorenyl group is excluded, and
when R_{d} is bonded to the nitrogen of Formula 1, Rₑ is a hydrogen atom or a deuterium atom, or combined with adjacent R_{f} to form a ring,
*-(L₁)ₘ-Ar₁, Formula 2-2
wherein in Formula 2-2,
L₁ is a substituted or unsubstituted arylene group of 6 to 30 ring-forming carbon atoms,
“m” is an integer of 0 to 3, and
Ar₁ is a substituted or unsubstituted aryl group of 6 to 30 ring-forming carbon atoms, where a fluorenyl group is excluded, and when R₂ and R₃ in Formula 1 are both represented by Formula 2-2, Ar₁ is not a 1-naphthyl group, 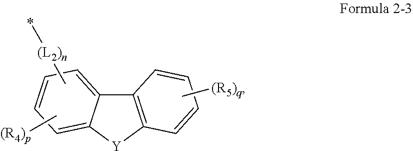
and
wherein in Formula 2-3,
Y is O or S,
L₂ is a substituted or unsubstituted arylene group of 6 to 30 ring-forming carbon atoms,
R₄ is a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring,
R₅ is a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring,
“n” and “p” are each independently an integer of 0 to 3, and
“q” is an integer of 0 to 4,
provided that Formula 2-1 and Formula 2-3 are not the same,
when Rₐ of Formula 2-1 is bonded to the nitrogen of Formula 1, and Rₕ is an alkyl group or an aryl group, at least one selected from among R₂ and R₃ of Formula 1 is represented by Formula 2-3,
when X of Formula 2-1 is S, Formula 2-3 is not a 4-dibenzothiophenyl group, and
at least one hydrogen in the amine compound represented by Formula 1 is optionally substituted with deuterium.

2. The luminescence device of claim 1, wherein the hole transport region comprises:
a hole injection layer on the first electrode; and
a hole transport layer on the hole injection layer, and
wherein the hole injection layer or the hole transport layer comprises the amine compound represented by Formula 1.

3. The luminescence device of claim 1, wherein the hole transport region comprises:
a hole transport layer on the first electrode; and
an electron blocking layer on the hole transport layer, and
wherein the electron blocking layer comprises the amine compound represented by Formula 1.

4. The luminescence device of claim 1, wherein:
R₁ of Formula 1 is represented by Formula 2-1-1,
R₂ of Formula 1 is represented by Formula 2-3, and
R₃ of Formula 1 is represented by Formula 2-2 or Formula 2-3: 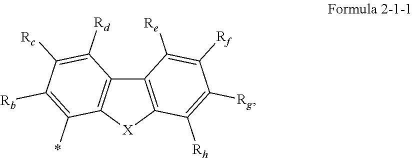
and
wherein in Formula 2-1-1,
X is O or S,
Rₕ is a substituted or unsubstituted alkyl group of 1 to 20 carbon atoms or a substituted or unsubstituted aryl group of 6 to 30 ring-forming carbon atoms, and
R_{b} to R_{g} are each independently a hydrogen atom, a deuterium atom, or a halogen atom, or combined with an adjacent group to form a ring.

5. The luminescence device of claim 1, wherein:
R₁ of Formula 1 is represented by Formula 2-1-2,
R₂ of Formula 1 is represented by Formula 2-2, and
R₃ of Formula 1 is represented by Formula 2-2 or Formula 2-3: 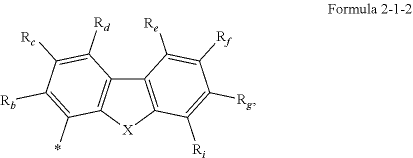
and
wherein in Formula 2-1-2,
X is O or S,
Rᵢ is a hydrogen atom or a deuterium atom, or combined with an adjacent R_{g} group to form a ring,
R_{b} to R_{d} are each independently a hydrogen atom, a deuterium atom, or a halogen atom, or combined with an adjacent group to form a ring, and
any one selected from among Rₑ to R_{g} is a substituted or unsubstituted alkyl group of 1 to 20 carbon atoms or a substituted or unsubstituted aryl group of 6 to 30 ring-forming carbon atoms, and the remainder are each independently a hydrogen atom, a deuterium atom, or a halogen atom, or combined with an adjacent group to form a ring, where a fluorenyl group is excluded.

6. The luminescence device of claim 1, wherein:
R₁ of Formula 1 is represented by Formula 2-1-2,
R₂ of Formula 1 is represented by Formula 2-3, and
R₃ of Formula 1 is represented by Formula 2-2 or Formula 2-3: 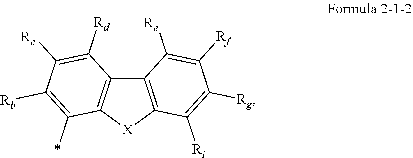
and
wherein in Formula 2-1-2,
X is O or S,
Rᵢ is a hydrogen atom or a deuterium atom, or combined with an adjacent R_{g} group to form a ring,
R_{b} to R_{d} are each independently a hydrogen atom, a deuterium atom, or a halogen atom, or combined with an adjacent group to form a ring, and
any one selected from among Rₑ to R_{g} is a substituted or unsubstituted alkyl group of 1 to 20 carbon atoms or a substituted or unsubstituted aryl group of 6 to 30 ring-forming carbon atoms, and the remainder are each independently a hydrogen atom, a deuterium atom, or a halogen atom, or combined with an adjacent group to form a ring, where a fluorenyl group is excluded.

7. The luminescence device of claim 1, wherein
R₁ of Formula 1 is represented by Formula 2-1-3, and
R₂ and R₃ of Formula 1 are each independently represented by Formula 2-2 or Formula 2-3: 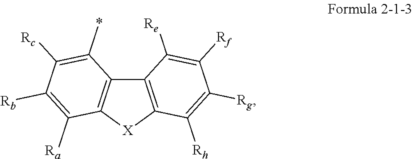
and
wherein in Formula 2-1-3,
X is O or S,
Rₑ is a hydrogen atom or a deuterium atom, or combined with an adjacent R_{f} group to form a ring,
Rₐ to R_{c} are each independently a hydrogen atom, a deuterium atom, or a halogen atom, or combined with an adjacent group to form a ring, and
any one selected from among R_{f} to Rₕ is a substituted or unsubstituted alkyl group of 1 to 20 carbon atoms or a substituted or unsubstituted aryl group of 6 to 30 ring-forming carbon atoms, and the remainder are each independently a hydrogen atom, a deuterium atom, or a halogen atom, or combined with an adjacent group to form a ring, where a fluorenyl group is excluded.

8. The luminescence device of claim 1, wherein Formula 1 is represented by Formula 3-1 or Formula 3-2: 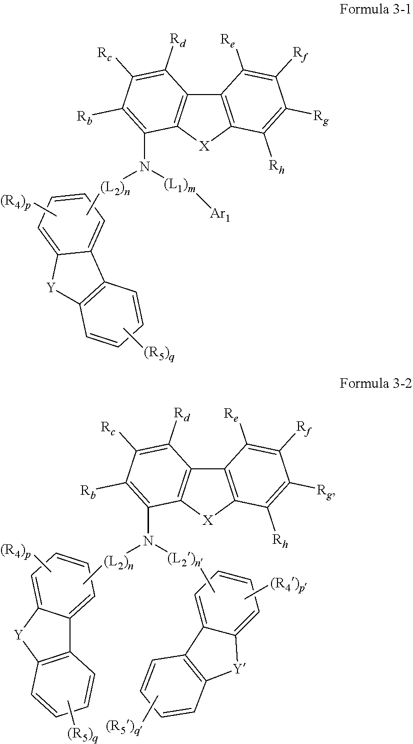
and
wherein in Formula 3-1 and Formula 3-2,
Rₕ is a substituted or unsubstituted alkyl group of 1 to 20 carbon atoms or a substituted or unsubstituted aryl group of 6 to 30 ring-forming carbon atoms,
R_{b} to R_{g} are each independently a hydrogen atom, a deuterium atom, or a halogen atom, or combined with an adjacent group to form a ring,
Y′ is O or S,
L₂′ is a substituted or unsubstituted arylene group of 6 to 30 ring-forming carbon atoms,
R₄′ is a hydrogen atom, a deuterium atom, a halogen atom, or a substituted or unsubstituted alkyl group of 1 to 20 carbon atoms, and/or combined with an adjacent group to form a ring,
R₅′ is a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring,
n′ and p′ are each independently an integer of 0 to 3,
q′ is an integer of 0 to 4, and
X, Ar₁, Y, L₁, L₂, R₄, R₅, “m”, “n”, “p” and “q” are each independently the same as defined in Formula 2-1 to Formula 2-3.

9. The luminescence device of claim 1, wherein Formula 1 is represented by any one selected from among Formula 4-1 to Formula 4-3: 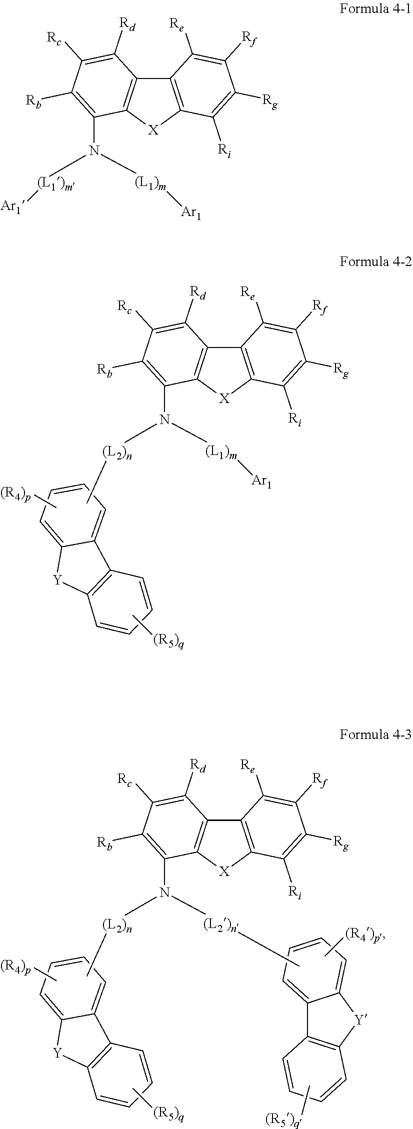
and
wherein in Formula 4-1 to Formula 4-3,
Rᵢ is a hydrogen atom or a deuterium atom, or combined with an adjacent R_{g} group to form a ring,
R_{b} to R_{d} are each independently a hydrogen atom, a deuterium atom, or a halogen atom, or combined with an adjacent group to form a ring,
any one selected from among Rₑ to R_{g} is a substituted or unsubstituted alkyl group of 1 to 20 carbon atoms or a substituted or unsubstituted aryl group of 6 to 30 ring-forming carbon atoms, and the remainder are each independently a hydrogen atom, a deuterium atom, or a halogen atom, or combined with an adjacent group to form a ring, where a fluorenyl group is excluded,
L₁′ and L₂′ are each independently a substituted or unsubstituted arylene group of 6 to 30 ring-forming carbon atoms,
R₄′ is a hydrogen atom, a deuterium atom, a halogen atom, or a substituted or unsubstituted alkyl group of 1 to 20 carbon atoms, and/or combined with an adjacent group to form a ring,
R₅′ is a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring,
n′ and p′ are each independently an integer of 0 to 3,
q′ is an integer of 0 to 4,
Ar₁ and Ar₁′ are each independently a substituted or unsubstituted aryl group of 6 to 30 ring-forming carbon atoms, where a fluorenyl group is excluded,
m′ is an integer of 0 to 3, and
X, Y, L₁, L₂, R₄, R₅, “m”, “n”, “p” and “q” are each independently the same as defined in Formula 2-1 to Formula 2-3, where Ar₁ and Ar₁′ in Formula 4-1 are not simultaneously 1-naphthyl groups.

10. The luminescence device of claim 1, wherein Formula 1 is represented by any one selected from among Formula 5-1 to Formula 5-3: 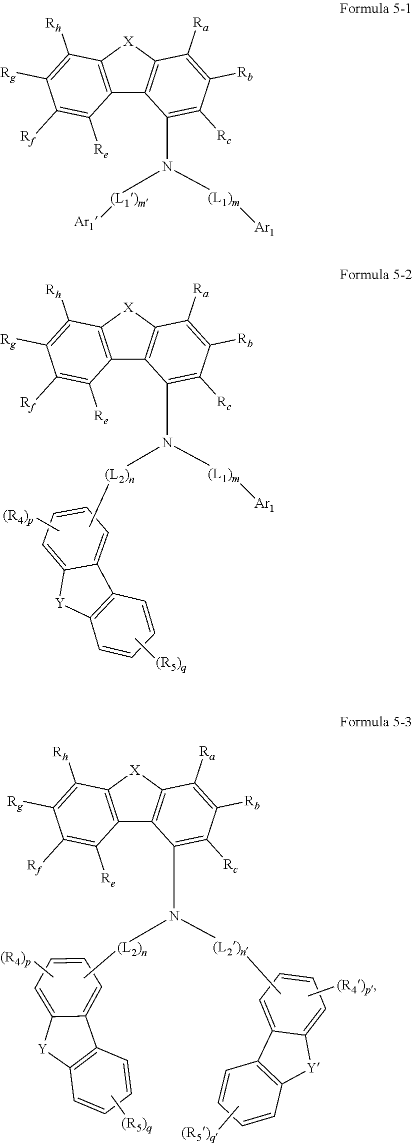
and
wherein in Formula 5-1 to Formula 5-3,
Rₑ is a hydrogen atom or a deuterium atom, or combined with an adjacent R_{f} group to form a ring,
Rₐ to R_{c} are each independently a hydrogen atom, a deuterium atom, or a halogen atom, or combined with an adjacent group to form a ring,
any one selected from among R_{f} to Rₕ is a substituted or unsubstituted alkyl group of 1 to 20 carbon atoms or a substituted or unsubstituted aryl group of 6 to 30 ring-forming carbon atoms, and the remainder are each independently a hydrogen atom, a deuterium atom, or a halogen atom, or combined with an adjacent group to form a ring, where a fluorenyl group is excluded,
L₁′ is a substituted or unsubstituted arylene group of 6 to 30 ring-forming carbon atoms,
Ar₁ and Ar₁′ are each independently a substituted or unsubstituted aryl group of 6 to 30 ring-forming carbon atoms, where a fluorenyl group is excluded,
m′ is an integer of 0 to 3,
Y′ is O or S,
L₂′ is a substituted or unsubstituted arylene group of 6 to 30 ring-forming carbon atoms,
R₄′ is a hydrogen atom, a deuterium atom, a halogen atom, or a substituted or unsubstituted alkyl group of 1 to 20 carbon atoms, and/or combined with an adjacent group to form a ring,
R₅′ is a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring,
n′ and p′ are each independently an integer of 0 to 3,
q′ is an integer of 0 to 4, and
X, Y, L₁, L₂, R₄, R₅, “m”, “n”, “p” and “q” are each independently the same as defined in Formula 2-1 to Formula 2-3, where Ar₁ and Ar₁′ in Formula 5-1 are not simultaneously 1-naphthyl groups.

11. The luminescence device of claim 1, wherein L₁ and L₂ of Formula 2-2 and Formula 2-3 are each independently a direct linkage, a substituted or unsubstituted phenylene group, a substituted or unsubstituted divalent biphenyl group, or a substituted or unsubstituted naphthylenyl group.

12. The luminescence device of claim 1, wherein the amine compound represented by Formula 1 is any one selected from among compounds represented in Compound Group 1:

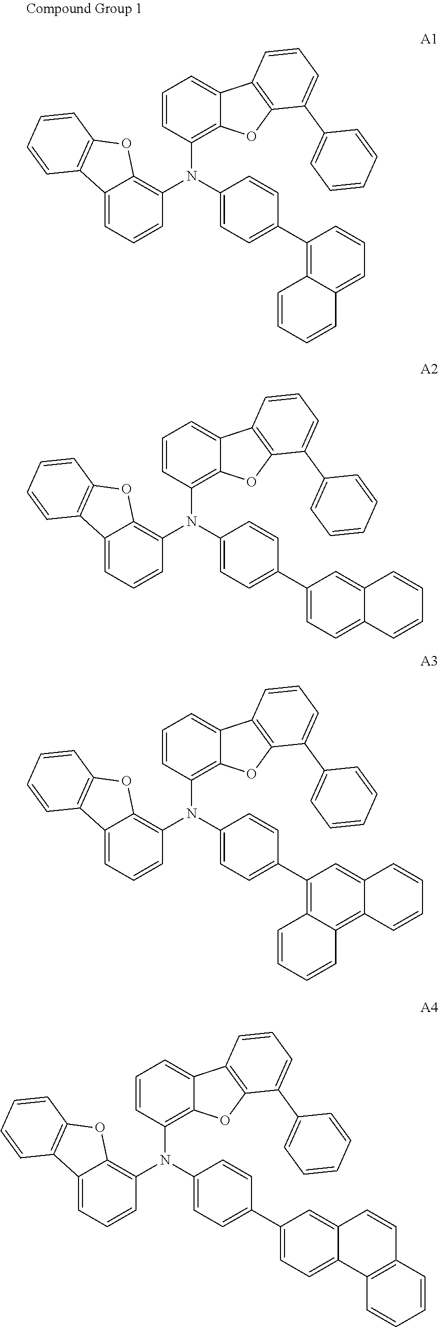 [SMILES: c1ccc(-c2cccc3c2oc2c(N(c4ccc(-c5cccc6ccccc56)cc4)c4cccc5c4oc4ccccc45)cccc23)cc1 ; c1ccc(-c2cccc3c2oc2c(N(c4ccc(-c5ccc6ccccc6c5)cc4)c4cccc5c4oc4ccccc45)cccc23)cc1 ; c1ccc(-c2cccc3c2oc2c(N(c4ccc(-c5cc6ccccc6c6ccccc56)cc4)c4cccc5c4oc4ccccc45)cccc23)cc1 ; c1ccc(-c2cccc3c2oc2c(N(c4ccc(-c5ccc6c(ccc7ccccc76)c5)cc4)c4cccc5c4oc4ccccc45)cccc23)cc1]

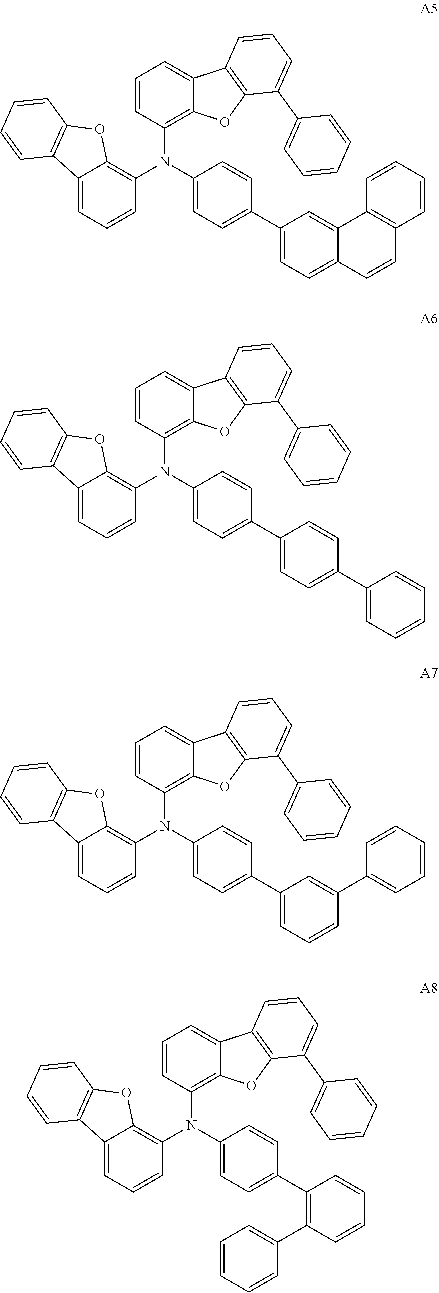 [SMILES: c1ccc(-c2cccc3c2oc2c(N(c4ccc(-c5ccc6ccc7ccccc7c6c5)cc4)c4cccc5c4oc4ccccc45)cccc23)cc1 ; c1ccc(-c2ccc(-c3ccc(N(c4cccc5c4oc4ccccc45)c4cccc5c4oc4c(-c6ccccc6)cccc45)cc3)cc2)cc1 ; c1ccc(-c2cccc(-c3ccc(N(c4cccc5c4oc4ccccc45)c4cccc5c4oc4c(-c6ccccc6)cccc45)cc3)c2)cc1 ; c1ccc(-c2ccccc2-c2ccc(N(c3cccc4c3oc3ccccc34)c3cccc4c3oc3c(-c5ccccc5)cccc34)cc2)cc1]

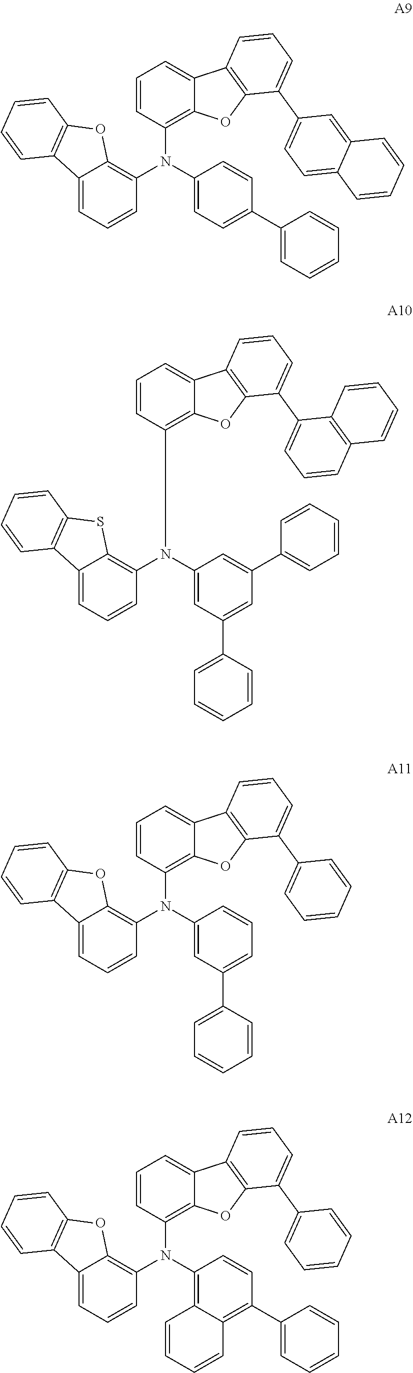 [SMILES: c1ccc(-c2ccc(N(c3cccc4c3oc3ccccc34)c3cccc4c3oc3c(-c5ccc6ccccc6c5)cccc34)cc2)cc1 ; c1ccc(-c2cc(-c3ccccc3)cc(N(c3cccc4c3oc3c(-c5cccc6ccccc56)cccc34)c3cccc4c3sc3ccccc34)c2)cc1 ; c1ccc(-c2cccc(N(c3cccc4c3oc3ccccc34)c3cccc4c3oc3c(-c5ccccc5)cccc34)c2)cc1 ; c1ccc(-c2ccc(N(c3cccc4c3oc3ccccc34)c3cccc4c3oc3c(-c5ccccc5)cccc34)c3ccccc23)cc1]

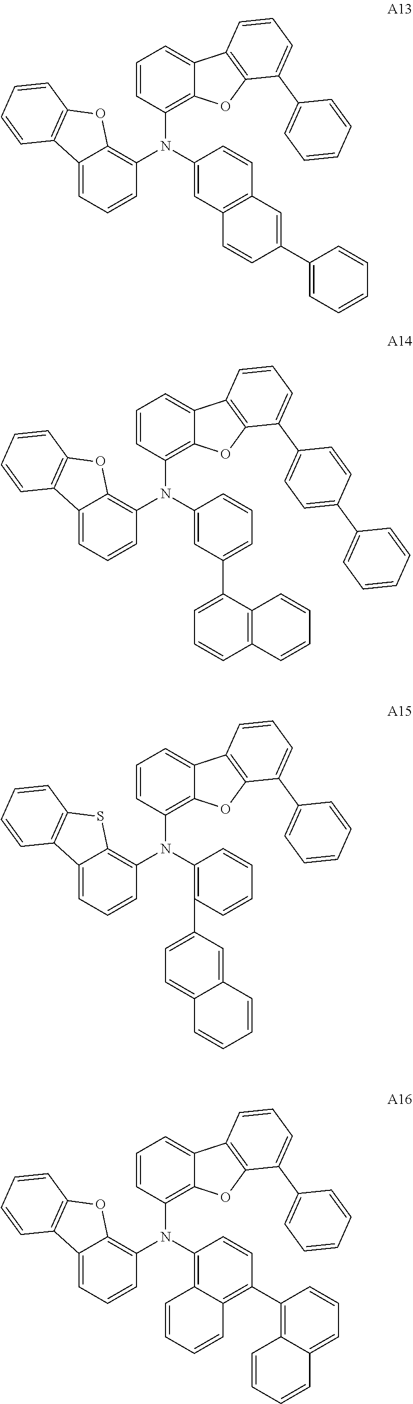 [SMILES: c1ccc(-c2ccc3cc(N(c4cccc5c4oc4ccccc45)c4cccc5c4oc4c(-c6ccccc6)cccc45)ccc3c2)cc1 ; c1ccc(-c2ccc(-c3cccc4c3oc3c(N(c5cccc(-c6cccc7ccccc67)c5)c5cccc6c5oc5ccccc56)cccc34)cc2)cc1 ; c1ccc(-c2cccc3c2oc2c(N(c4ccccc4-c4ccc5ccccc5c4)c4cccc5c4sc4ccccc45)cccc23)cc1 ; c1ccc(-c2cccc3c2oc2c(N(c4ccc(-c5cccc6ccccc56)c5ccccc45)c4cccc5c4oc4ccccc45)cccc23)cc1]

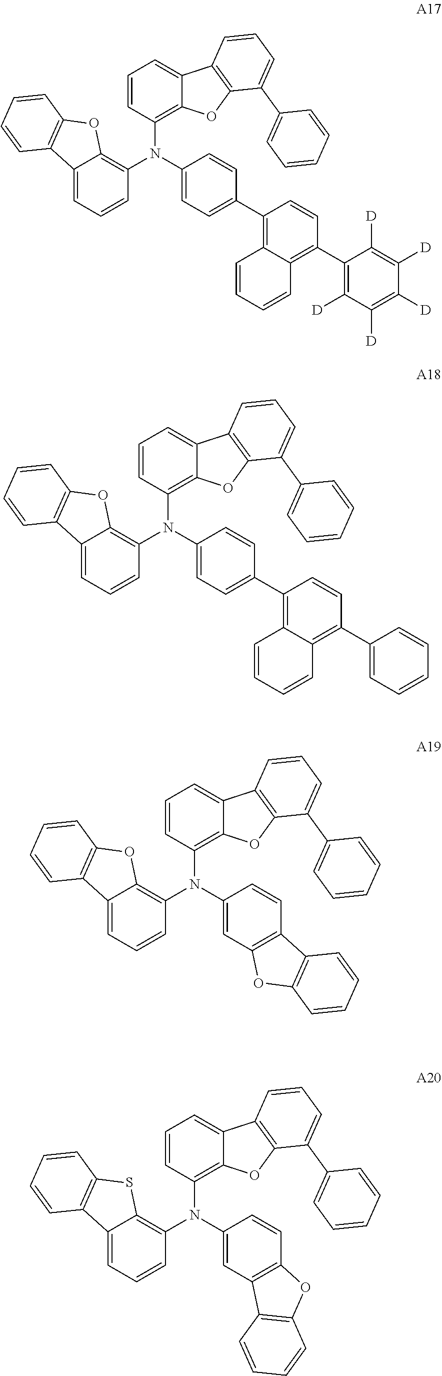 [SMILES: [2H]c1c([2H])c([2H])c(-c2ccc(-c3ccc(N(c4cccc5c4oc4ccccc45)c4cccc5c4oc4c(-c6ccccc6)cccc45)cc3)c3ccccc23)c([2H])c1[2H] ; c1ccc(-c2ccc(-c3ccc(N(c4cccc5c4oc4ccccc45)c4cccc5c4oc4c(-c6ccccc6)cccc45)cc3)c3ccccc23)cc1 ; c1ccc(-c2cccc3c2oc2c(N(c4ccc5c(c4)oc4ccccc45)c4cccc5c4oc4ccccc45)cccc23)cc1 ; c1ccc(-c2cccc3c2oc2c(N(c4ccc5oc6ccccc6c5c4)c4cccc5c4sc4ccccc45)cccc23)cc1]

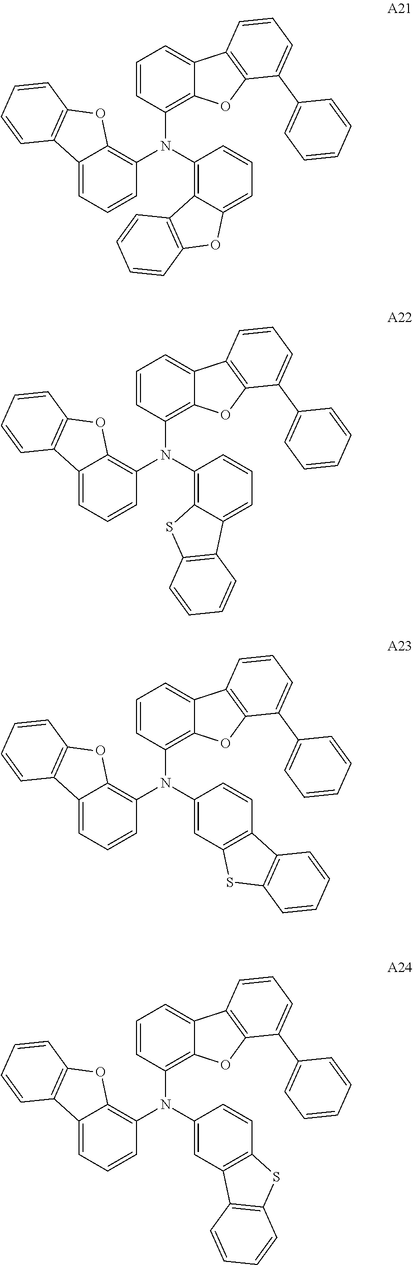 [SMILES: c1ccc(-c2cccc3c2oc2c(N(c4cccc5c4oc4ccccc45)c4cccc5oc6ccccc6c45)cccc23)cc1 ; c1ccc(-c2cccc3c2oc2c(N(c4cccc5c4oc4ccccc45)c4cccc5c4sc4ccccc45)cccc23)cc1 ; c1ccc(-c2cccc3c2oc2c(N(c4ccc5c(c4)sc4ccccc45)c4cccc5c4oc4ccccc45)cccc23)cc1 ; c1ccc(-c2cccc3c2oc2c(N(c4ccc5sc6ccccc6c5c4)c4cccc5c4oc4ccccc45)cccc23)cc1]

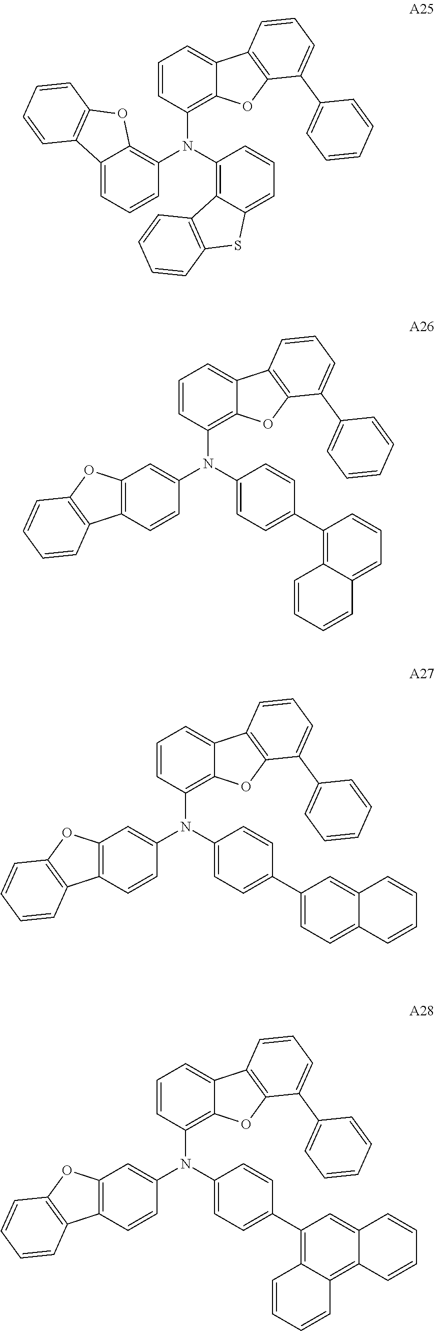 [SMILES: c1ccc(-c2cccc3c2oc2c(N(c4cccc5c4oc4ccccc45)c4cccc5sc6ccccc6c45)cccc23)cc1 ; c1ccc(-c2cccc3c2oc2c(N(c4ccc(-c5cccc6ccccc56)cc4)c4ccc5c(c4)oc4ccccc45)cccc23)cc1 ; c1ccc(-c2cccc3c2oc2c(N(c4ccc(-c5ccc6ccccc6c5)cc4)c4ccc5c(c4)oc4ccccc45)cccc23)cc1 ; c1ccc(-c2cccc3c2oc2c(N(c4ccc(-c5cc6ccccc6c6ccccc56)cc4)c4ccc5c(c4)oc4ccccc45)cccc23)cc1]

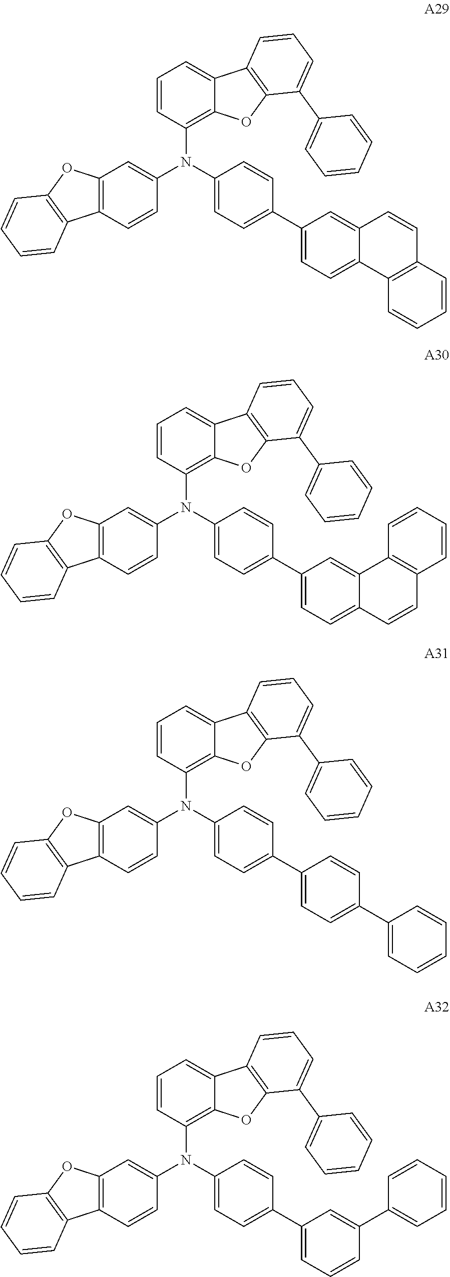 [SMILES: c1ccc(-c2cccc3c2oc2c(N(c4ccc(-c5ccc6c(ccc7ccccc76)c5)cc4)c4ccc5c(c4)oc4ccccc45)cccc23)cc1 ; c1ccc(-c2cccc3c2oc2c(N(c4ccc(-c5ccc6ccc7ccccc7c6c5)cc4)c4ccc5c(c4)oc4ccccc45)cccc23)cc1 ; c1ccc(-c2ccc(-c3ccc(N(c4ccc5c(c4)oc4ccccc45)c4cccc5c4oc4c(-c6ccccc6)cccc45)cc3)cc2)cc1 ; c1ccc(-c2cccc(-c3ccc(N(c4ccc5c(c4)oc4ccccc45)c4cccc5c4oc4c(-c6ccccc6)cccc45)cc3)c2)cc1]

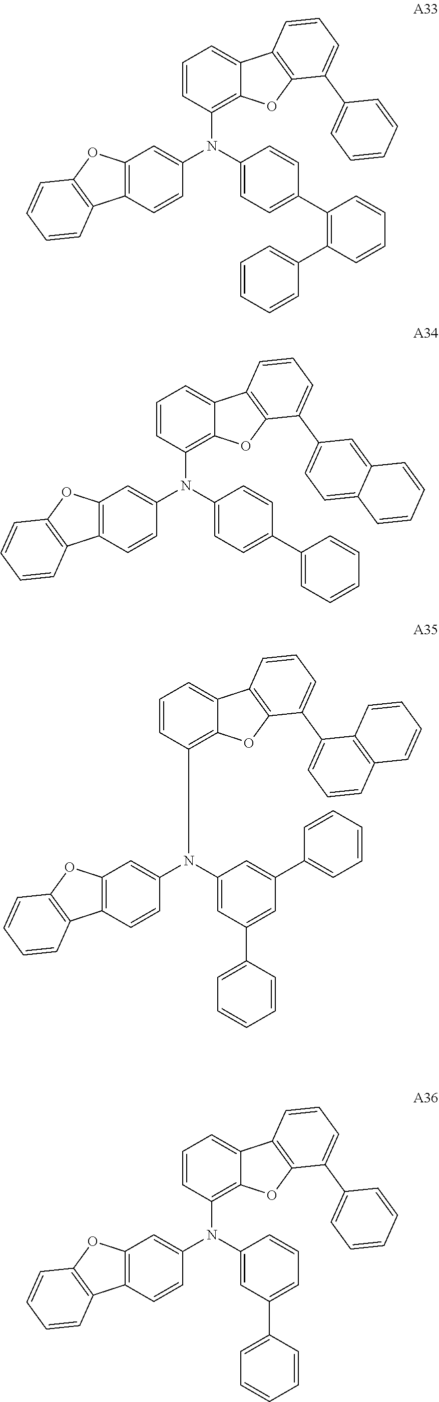 [SMILES: c1ccc(-c2ccccc2-c2ccc(N(c3ccc4c(c3)oc3ccccc34)c3cccc4c3oc3c(-c5ccccc5)cccc34)cc2)cc1 ; c1ccc(-c2ccc(N(c3ccc4c(c3)oc3ccccc34)c3cccc4c3oc3c(-c5ccc6ccccc6c5)cccc34)cc2)cc1 ; c1ccc(-c2cc(-c3ccccc3)cc(N(c3ccc4c(c3)oc3ccccc34)c3cccc4c3oc3c(-c5cccc6ccccc56)cccc34)c2)cc1 ; c1ccc(-c2cccc(N(c3ccc4c(c3)oc3ccccc34)c3cccc4c3oc3c(-c5ccccc5)cccc34)c2)cc1]

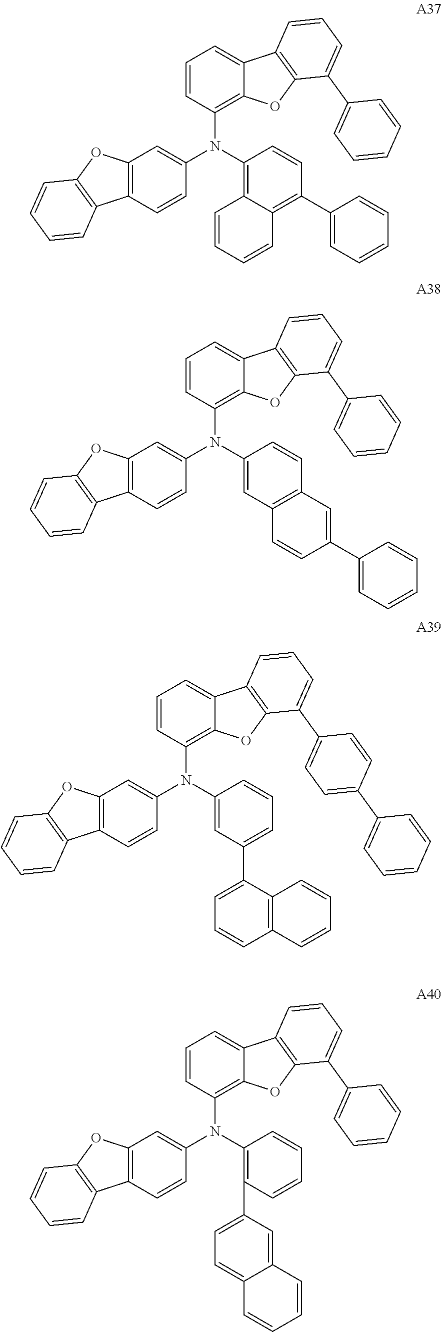 [SMILES: c1ccc(-c2ccc(N(c3ccc4c(c3)oc3ccccc34)c3cccc4c3oc3c(-c5ccccc5)cccc34)c3ccccc23)cc1 ; c1ccc(-c2ccc3cc(N(c4ccc5c(c4)oc4ccccc45)c4cccc5c4oc4c(-c6ccccc6)cccc45)ccc3c2)cc1 ; c1ccc(-c2ccc(-c3cccc4c3oc3c(N(c5cccc(-c6cccc7ccccc67)c5)c5ccc6c(c5)oc5ccccc56)cccc34)cc2)cc1 ; c1ccc(-c2cccc3c2oc2c(N(c4ccc5c(c4)oc4ccccc45)c4ccccc4-c4ccc5ccccc5c4)cccc23)cc1]

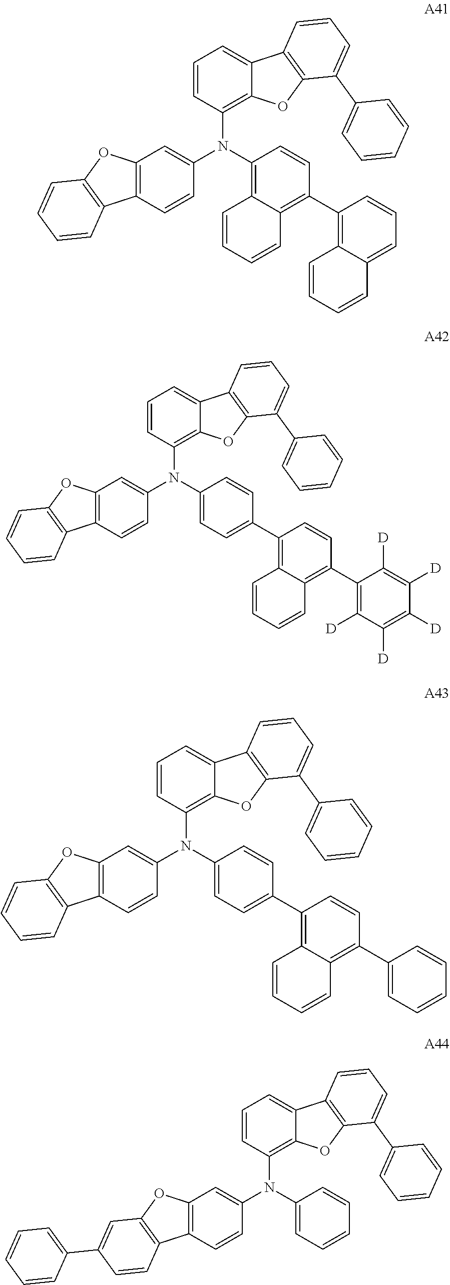 [SMILES: c1ccc(-c2cccc3c2oc2c(N(c4ccc5c(c4)oc4ccccc45)c4ccc(-c5cccc6ccccc56)c5ccccc45)cccc23)cc1 ; [2H]c1c([2H])c([2H])c(-c2ccc(-c3ccc(N(c4ccc5c(c4)oc4ccccc45)c4cccc5c4oc4c(-c6ccccc6)cccc45)cc3)c3ccccc23)c([2H])c1[2H] ; c1ccc(-c2ccc(-c3ccc(N(c4ccc5c(c4)oc4ccccc45)c4cccc5c4oc4c(-c6ccccc6)cccc45)cc3)c3ccccc23)cc1 ; c1ccc(-c2ccc3c(c2)oc2cc(N(c4ccccc4)c4cccc5c4oc4c(-c6ccccc6)cccc45)ccc23)cc1]

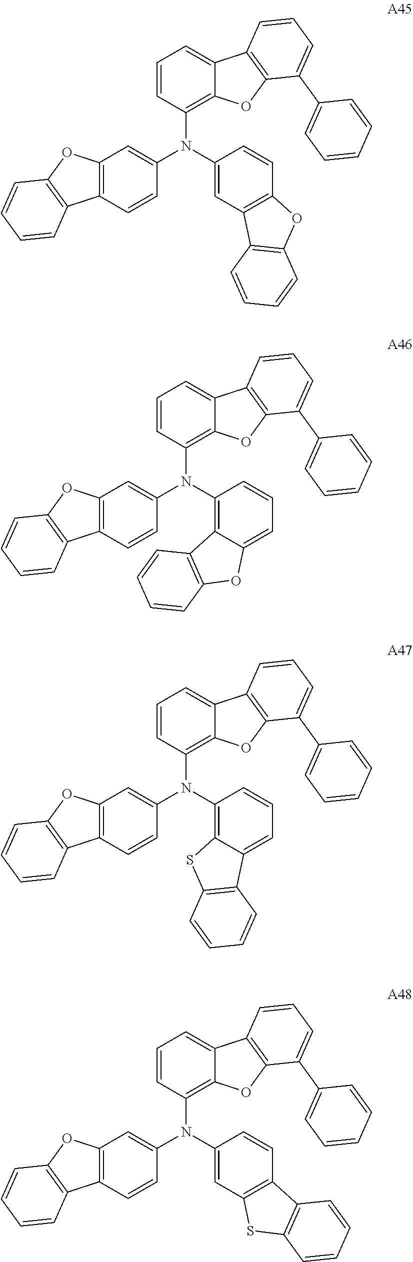 [SMILES: c1ccc(-c2cccc3c2oc2c(N(c4ccc5c(c4)oc4ccccc45)c4ccc5oc6ccccc6c5c4)cccc23)cc1 ; c1ccc(-c2cccc3c2oc2c(N(c4ccc5c(c4)oc4ccccc45)c4cccc5oc6ccccc6c45)cccc23)cc1 ; c1ccc(-c2cccc3c2oc2c(N(c4ccc5c(c4)oc4ccccc45)c4cccc5c4sc4ccccc45)cccc23)cc1 ; c1ccc(-c2cccc3c2oc2c(N(c4ccc5c(c4)oc4ccccc45)c4ccc5c(c4)sc4ccccc45)cccc23)cc1]

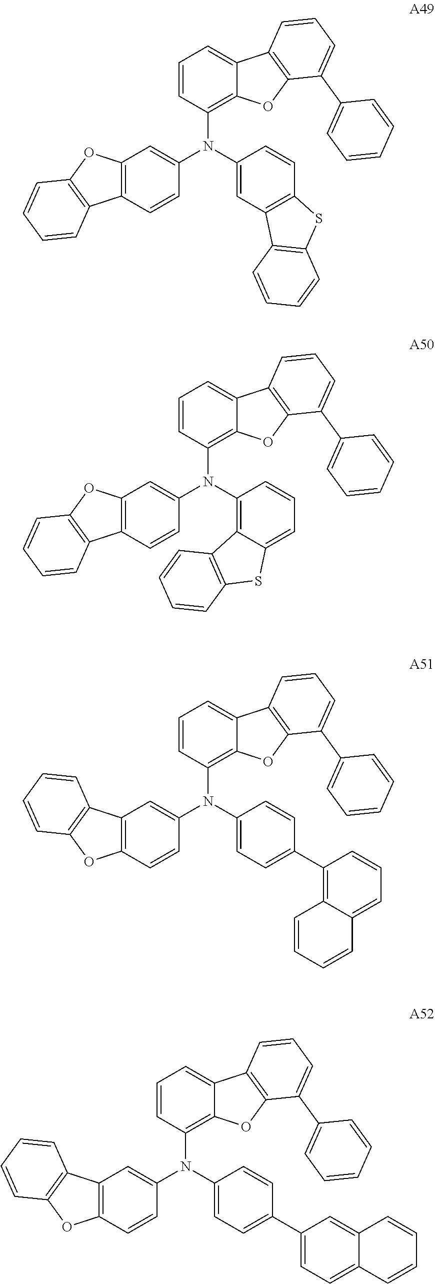 [SMILES: c1ccc(-c2cccc3c2oc2c(N(c4ccc5c(c4)oc4ccccc45)c4ccc5sc6ccccc6c5c4)cccc23)cc1 ; c1ccc(-c2cccc3c2oc2c(N(c4ccc5c(c4)oc4ccccc45)c4cccc5sc6ccccc6c45)cccc23)cc1 ; c1ccc(-c2cccc3c2oc2c(N(c4ccc(-c5cccc6ccccc56)cc4)c4ccc5oc6ccccc6c5c4)cccc23)cc1 ; c1ccc(-c2cccc3c2oc2c(N(c4ccc(-c5ccc6ccccc6c5)cc4)c4ccc5oc6ccccc6c5c4)cccc23)cc1]

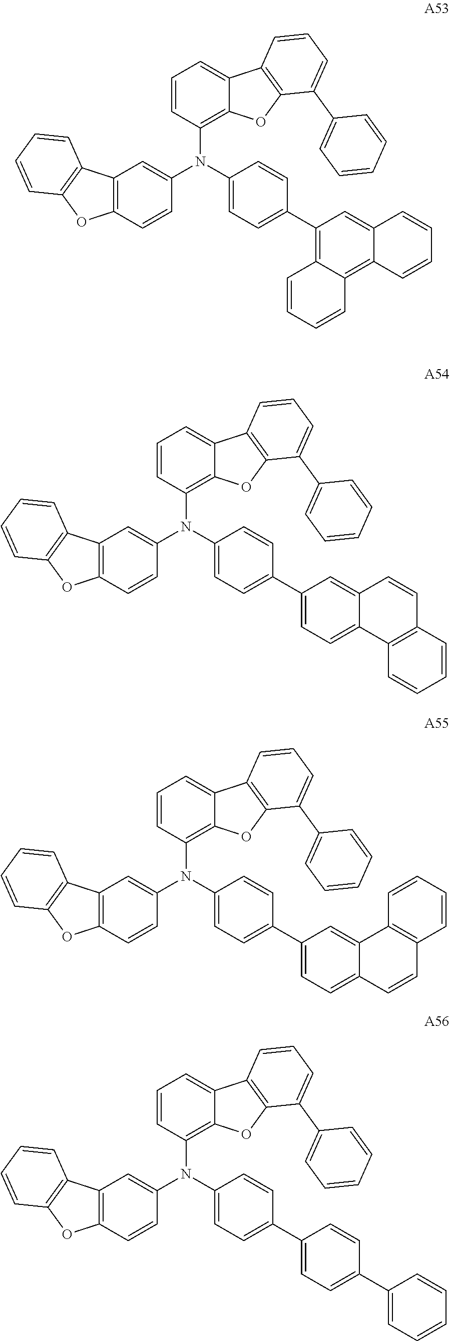 [SMILES: c1ccc(-c2cccc3c2oc2c(N(c4ccc(-c5cc6ccccc6c6ccccc56)cc4)c4ccc5oc6ccccc6c5c4)cccc23)cc1 ; c1ccc(-c2cccc3c2oc2c(N(c4ccc(-c5ccc6c(ccc7ccccc76)c5)cc4)c4ccc5oc6ccccc6c5c4)cccc23)cc1 ; c1ccc(-c2cccc3c2oc2c(N(c4ccc(-c5ccc6ccc7ccccc7c6c5)cc4)c4ccc5oc6ccccc6c5c4)cccc23)cc1 ; c1ccc(-c2ccc(-c3ccc(N(c4ccc5oc6ccccc6c5c4)c4cccc5c4oc4c(-c6ccccc6)cccc45)cc3)cc2)cc1]

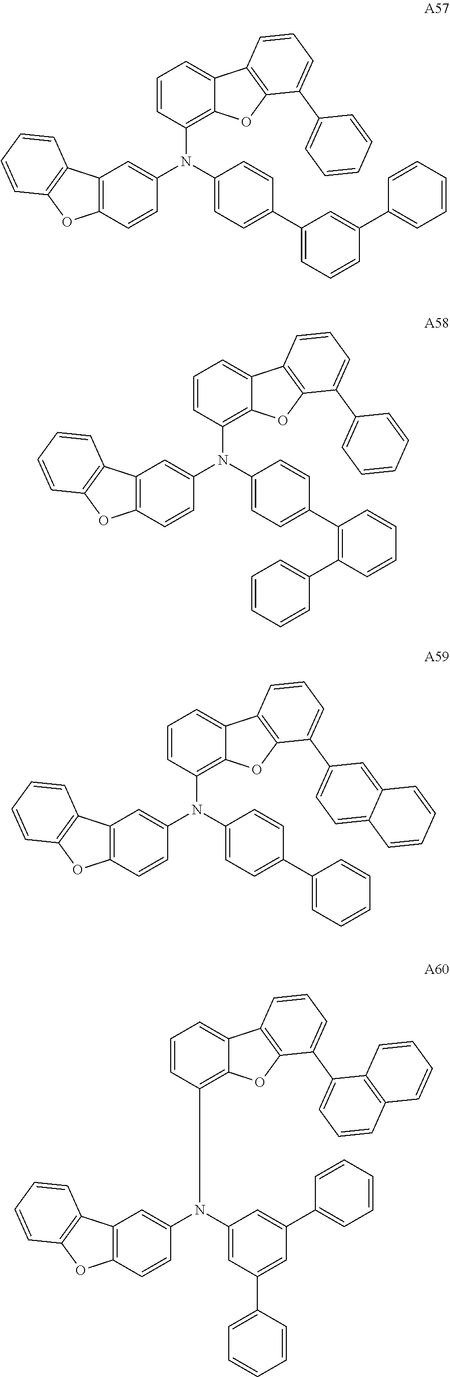 [SMILES: c1ccc(-c2cccc(-c3ccc(N(c4ccc5oc6ccccc6c5c4)c4cccc5c4oc4c(-c6ccccc6)cccc45)cc3)c2)cc1 ; c1ccc(-c2ccccc2-c2ccc(N(c3ccc4oc5ccccc5c4c3)c3cccc4c3oc3c(-c5ccccc5)cccc34)cc2)cc1 ; c1ccc(-c2ccc(N(c3ccc4oc5ccccc5c4c3)c3cccc4c3oc3c(-c5ccc6ccccc6c5)cccc34)cc2)cc1 ; c1ccc(-c2cc(-c3ccccc3)cc(N(c3ccc4oc5ccccc5c4c3)c3cccc4c3oc3c(-c5cccc6ccccc56)cccc34)c2)cc1]

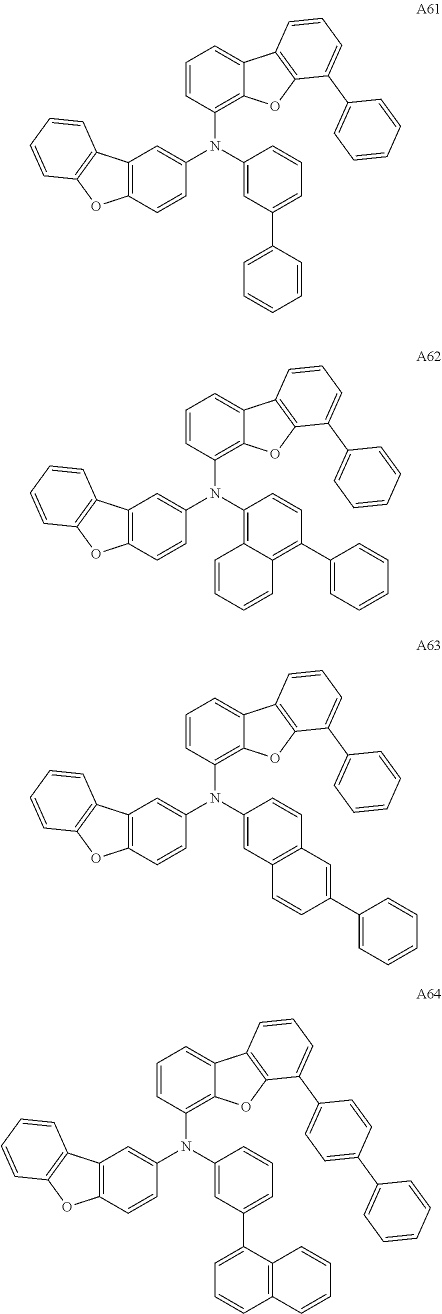 [SMILES: c1ccc(-c2cccc(N(c3ccc4oc5ccccc5c4c3)c3cccc4c3oc3c(-c5ccccc5)cccc34)c2)cc1 ; c1ccc(-c2ccc(N(c3ccc4oc5ccccc5c4c3)c3cccc4c3oc3c(-c5ccccc5)cccc34)c3ccccc23)cc1 ; c1ccc(-c2ccc3cc(N(c4ccc5oc6ccccc6c5c4)c4cccc5c4oc4c(-c6ccccc6)cccc45)ccc3c2)cc1 ; c1ccc(-c2ccc(-c3cccc4c3oc3c(N(c5cccc(-c6cccc7ccccc67)c5)c5ccc6oc7ccccc7c6c5)cccc34)cc2)cc1]

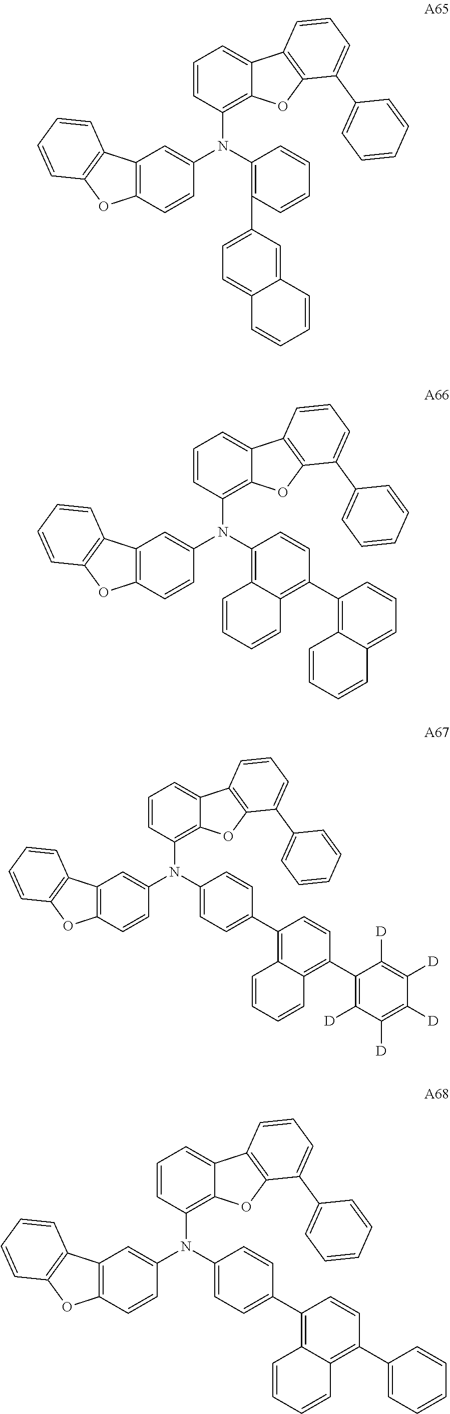 [SMILES: c1ccc(-c2cccc3c2oc2c(N(c4ccc5oc6ccccc6c5c4)c4ccccc4-c4ccc5ccccc5c4)cccc23)cc1 ; c1ccc(-c2cccc3c2oc2c(N(c4ccc5oc6ccccc6c5c4)c4ccc(-c5cccc6ccccc56)c5ccccc45)cccc23)cc1 ; [2H]c1c([2H])c([2H])c(-c2ccc(-c3ccc(N(c4ccc5oc6ccccc6c5c4)c4cccc5c4oc4c(-c6ccccc6)cccc45)cc3)c3ccccc23)c([2H])c1[2H] ; c1ccc(-c2ccc(-c3ccc(N(c4ccc5oc6ccccc6c5c4)c4cccc5c4oc4c(-c6ccccc6)cccc45)cc3)c3ccccc23)cc1]

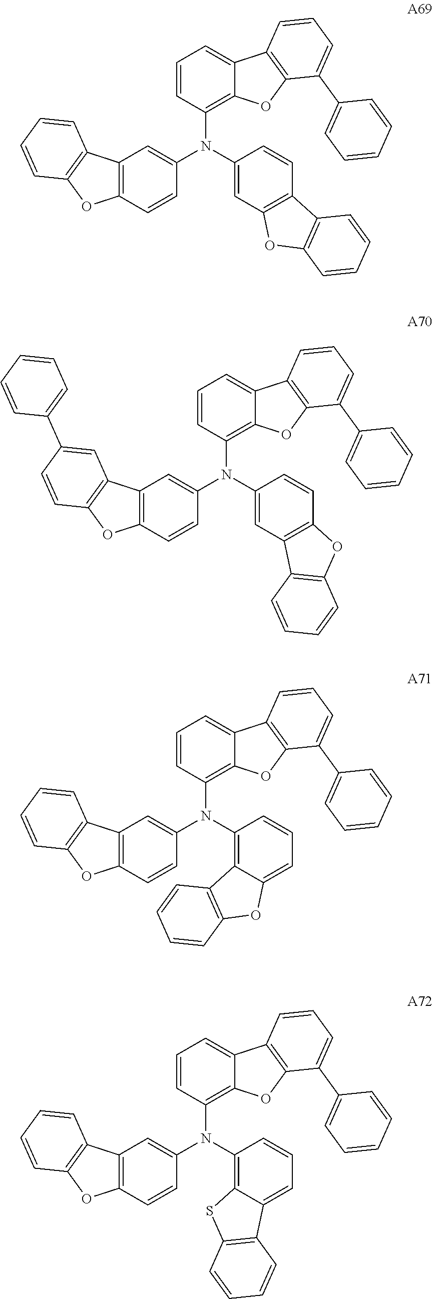 [SMILES: c1ccc(-c2cccc3c2oc2c(N(c4ccc5c(c4)oc4ccccc45)c4ccc5oc6ccccc6c5c4)cccc23)cc1 ; c1ccc(-c2ccc3oc4ccc(N(c5ccc6oc7ccccc7c6c5)c5cccc6c5oc5c(-c7ccccc7)cccc56)cc4c3c2)cc1 ; c1ccc(-c2cccc3c2oc2c(N(c4ccc5oc6ccccc6c5c4)c4cccc5oc6ccccc6c45)cccc23)cc1 ; c1ccc(-c2cccc3c2oc2c(N(c4ccc5oc6ccccc6c5c4)c4cccc5c4sc4ccccc45)cccc23)cc1]

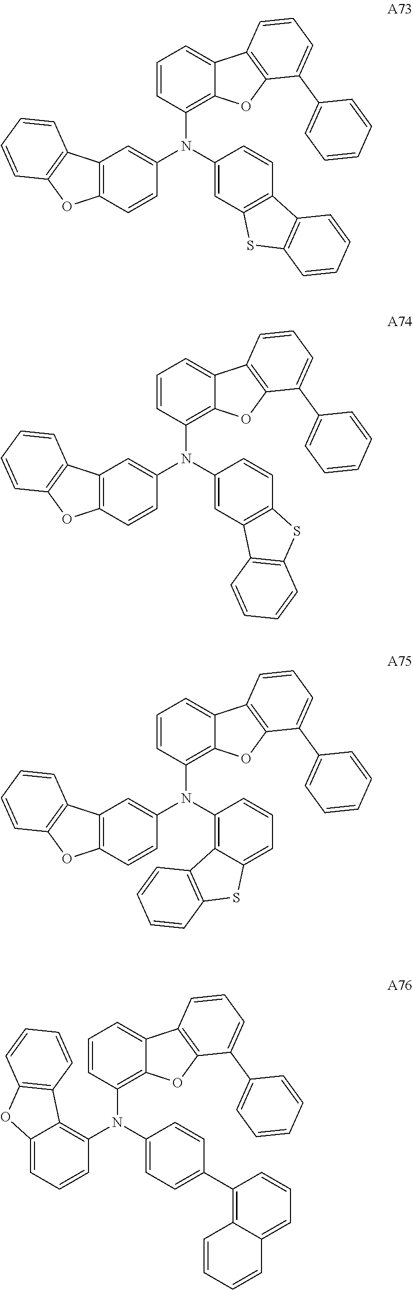 [SMILES: c1ccc(-c2cccc3c2oc2c(N(c4ccc5c(c4)sc4ccccc45)c4ccc5oc6ccccc6c5c4)cccc23)cc1 ; c1ccc(-c2cccc3c2oc2c(N(c4ccc5oc6ccccc6c5c4)c4ccc5sc6ccccc6c5c4)cccc23)cc1 ; c1ccc(-c2cccc3c2oc2c(N(c4ccc5oc6ccccc6c5c4)c4cccc5sc6ccccc6c45)cccc23)cc1 ; c1ccc(-c2cccc3c2oc2c(N(c4ccc(-c5cccc6ccccc56)cc4)c4cccc5oc6ccccc6c45)cccc23)cc1]

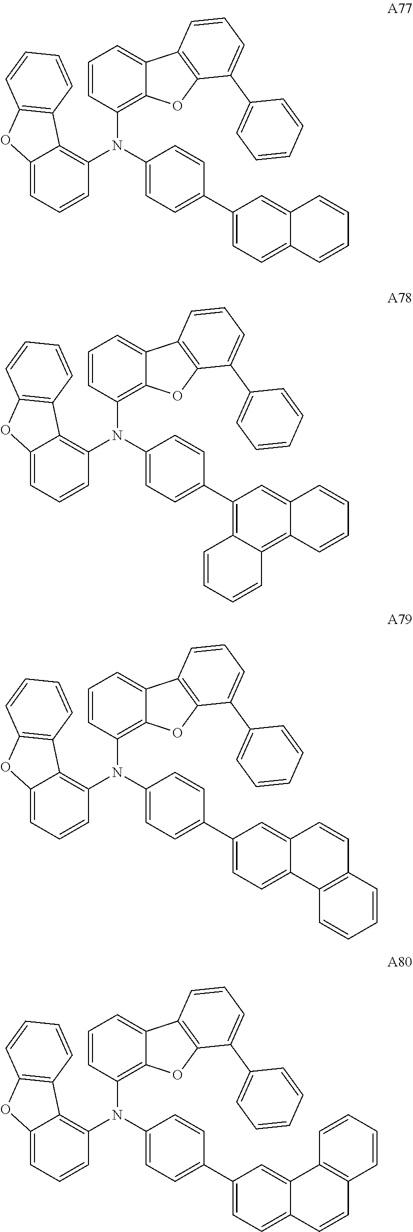

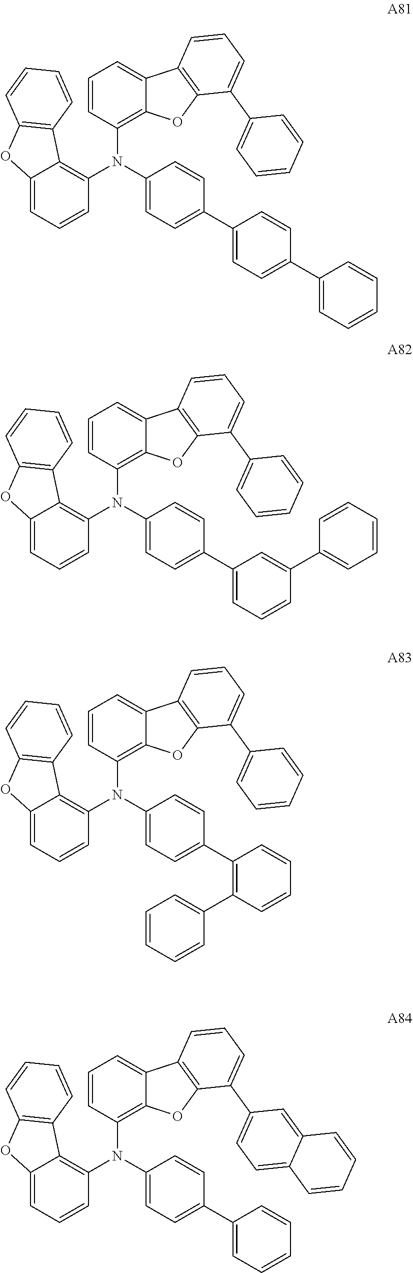 [SMILES: c1ccc(-c2ccc(-c3ccc(N(c4cccc5c4oc4c(-c6ccccc6)cccc45)c4cccc5oc6ccccc6c45)cc3)cc2)cc1 ; c1ccc(-c2cccc(-c3ccc(N(c4cccc5c4oc4c(-c6ccccc6)cccc45)c4cccc5oc6ccccc6c45)cc3)c2)cc1 ; c1ccc(-c2ccccc2-c2ccc(N(c3cccc4c3oc3c(-c5ccccc5)cccc34)c3cccc4oc5ccccc5c34)cc2)cc1 ; c1ccc(-c2ccc(N(c3cccc4c3oc3c(-c5ccc6ccccc6c5)cccc34)c3cccc4oc5ccccc5c34)cc2)cc1]

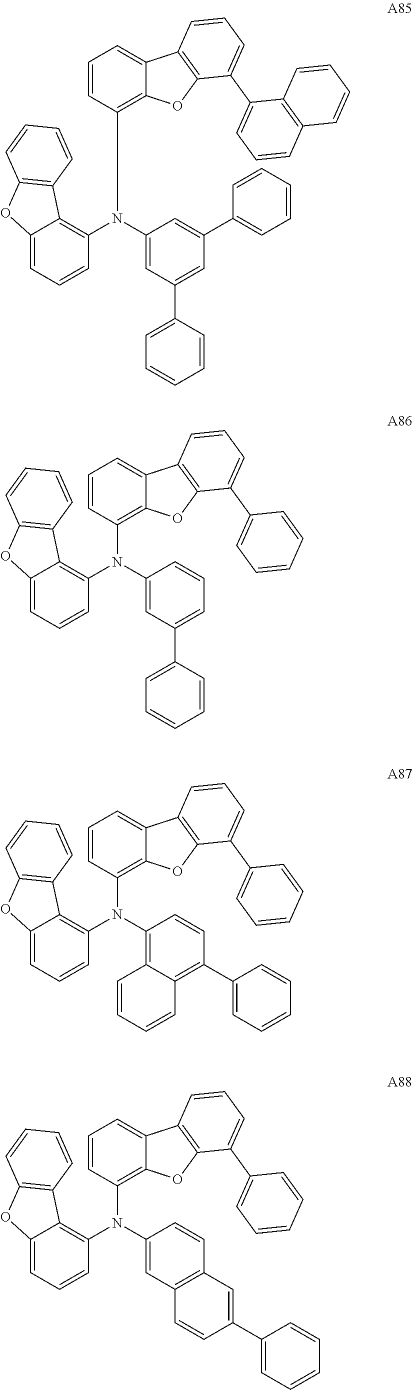 [SMILES: c1ccc(-c2cc(-c3ccccc3)cc(N(c3cccc4c3oc3c(-c5cccc6ccccc56)cccc34)c3cccc4oc5ccccc5c34)c2)cc1 ; c1ccc(-c2cccc(N(c3cccc4c3oc3c(-c5ccccc5)cccc34)c3cccc4oc5ccccc5c34)c2)cc1 ; c1ccc(-c2ccc(N(c3cccc4c3oc3c(-c5ccccc5)cccc34)c3cccc4oc5ccccc5c34)c3ccccc23)cc1 ; c1ccc(-c2ccc3cc(N(c4cccc5c4oc4c(-c6ccccc6)cccc45)c4cccc5oc6ccccc6c45)ccc3c2)cc1]

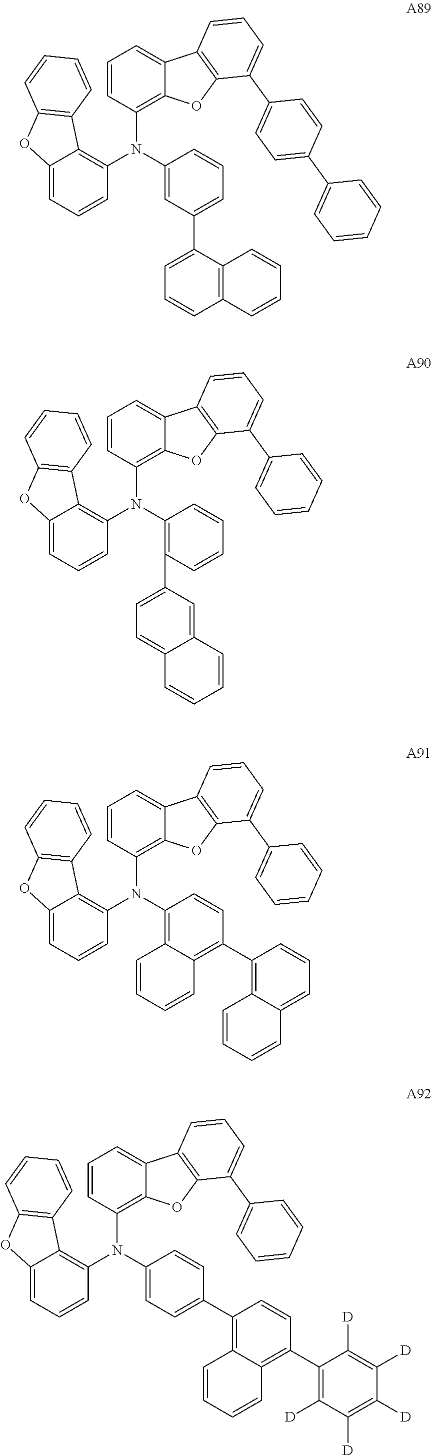 [SMILES: c1ccc(-c2ccc(-c3cccc4c3oc3c(N(c5cccc(-c6cccc7ccccc67)c5)c5cccc6oc7ccccc7c56)cccc34)cc2)cc1 ; c1ccc(-c2cccc3c2oc2c(N(c4ccccc4-c4ccc5ccccc5c4)c4cccc5oc6ccccc6c45)cccc23)cc1 ; c1ccc(-c2cccc3c2oc2c(N(c4ccc(-c5cccc6ccccc56)c5ccccc45)c4cccc5oc6ccccc6c45)cccc23)cc1 ; [2H]c1c([2H])c([2H])c(-c2ccc(-c3ccc(N(c4cccc5c4oc4c(-c6ccccc6)cccc45)c4cccc5oc6ccccc6c45)cc3)c3ccccc23)c([2H])c1[2H]]

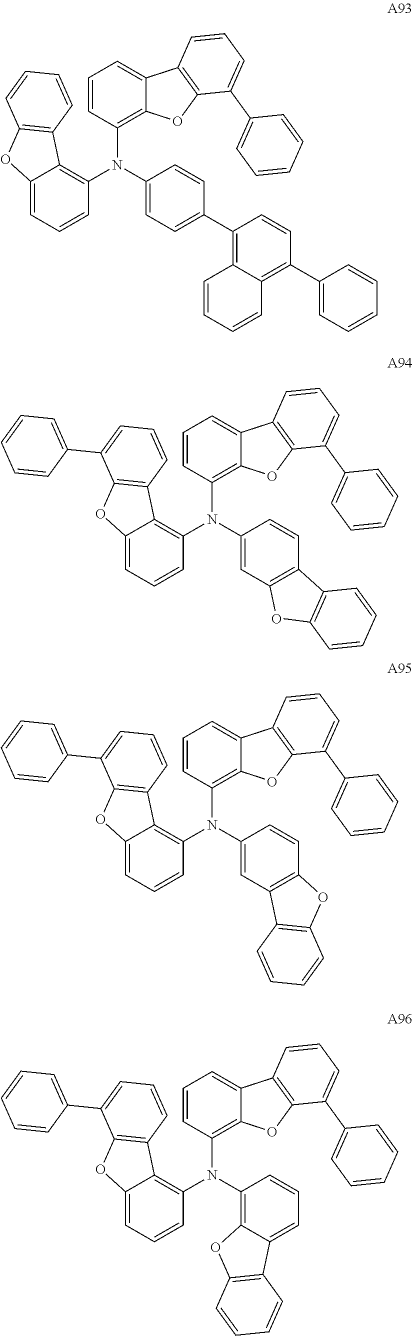

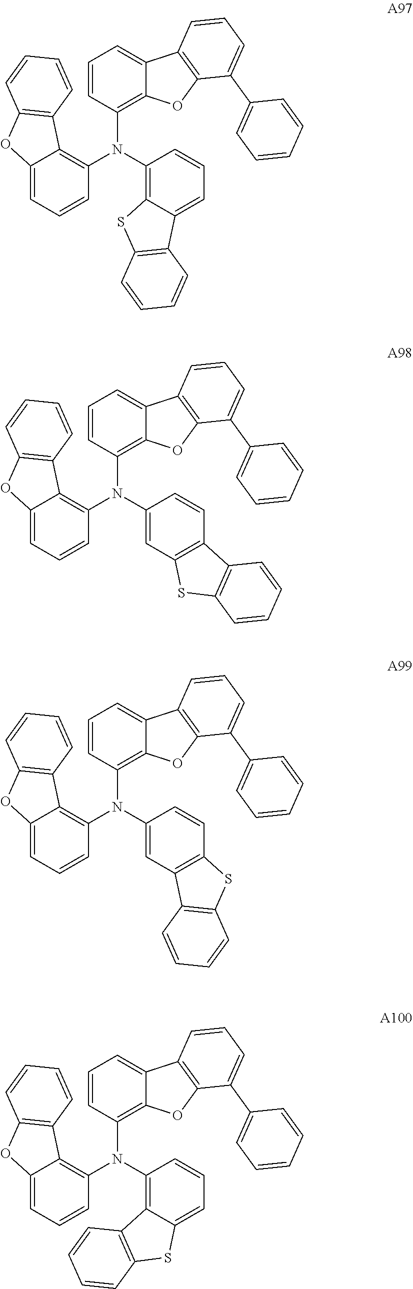 [SMILES: c1ccc(-c2cccc3c2oc2c(N(c4cccc5c4sc4ccccc45)c4cccc5oc6ccccc6c45)cccc23)cc1 ; c1ccc(-c2cccc3c2oc2c(N(c4ccc5c(c4)sc4ccccc45)c4cccc5oc6ccccc6c45)cccc23)cc1 ; c1ccc(-c2cccc3c2oc2c(N(c4ccc5sc6ccccc6c5c4)c4cccc5oc6ccccc6c45)cccc23)cc1 ; c1ccc(-c2cccc3c2oc2c(N(c4cccc5oc6ccccc6c45)c4cccc5sc6ccccc6c45)cccc23)cc1]

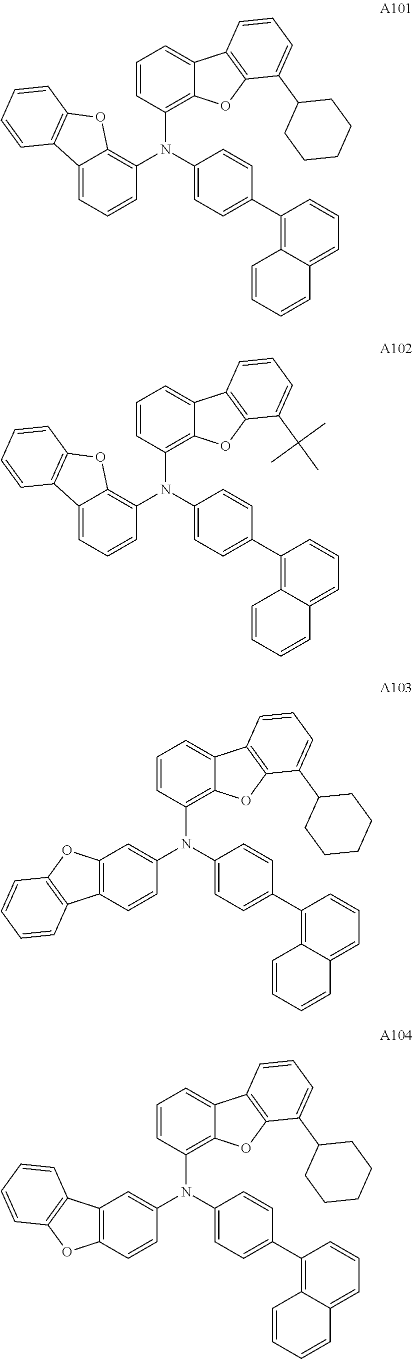

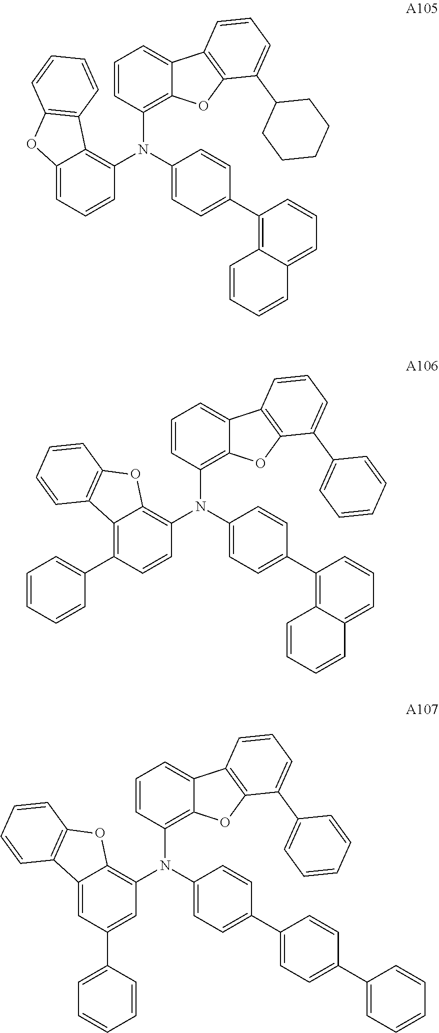 [SMILES: c1ccc2c(-c3ccc(N(c4cccc5c4oc4c(C6CCCCC6)cccc45)c4cccc5oc6ccccc6c45)cc3)cccc2c1 ; c1ccc(-c2cccc3c2oc2c(N(c4ccc(-c5cccc6ccccc56)cc4)c4ccc(-c5ccccc5)c5c4oc4ccccc45)cccc23)cc1 ; c1ccc(-c2ccc(-c3ccc(N(c4cc(-c5ccccc5)cc5c4oc4ccccc45)c4cccc5c4oc4c(-c6ccccc6)cccc45)cc3)cc2)cc1]

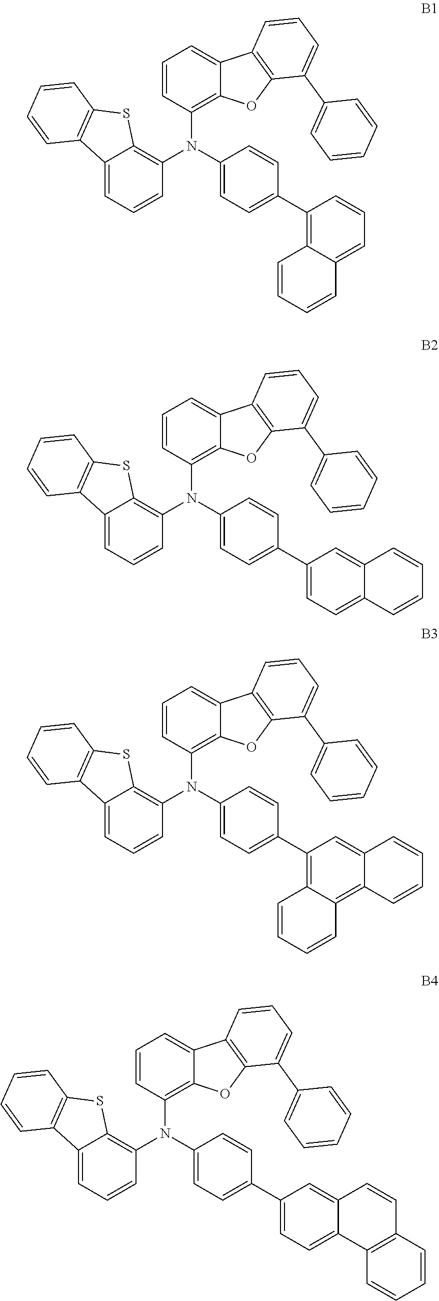 [SMILES: c1ccc(-c2cccc3c2oc2c(N(c4ccc(-c5cccc6ccccc56)cc4)c4cccc5c4sc4ccccc45)cccc23)cc1 ; c1ccc(-c2cccc3c2oc2c(N(c4ccc(-c5ccc6ccccc6c5)cc4)c4cccc5c4sc4ccccc45)cccc23)cc1 ; c1ccc(-c2cccc3c2oc2c(N(c4ccc(-c5cc6ccccc6c6ccccc56)cc4)c4cccc5c4sc4ccccc45)cccc23)cc1 ; c1ccc(-c2cccc3c2oc2c(N(c4ccc(-c5ccc6c(ccc7ccccc76)c5)cc4)c4cccc5c4sc4ccccc45)cccc23)cc1]

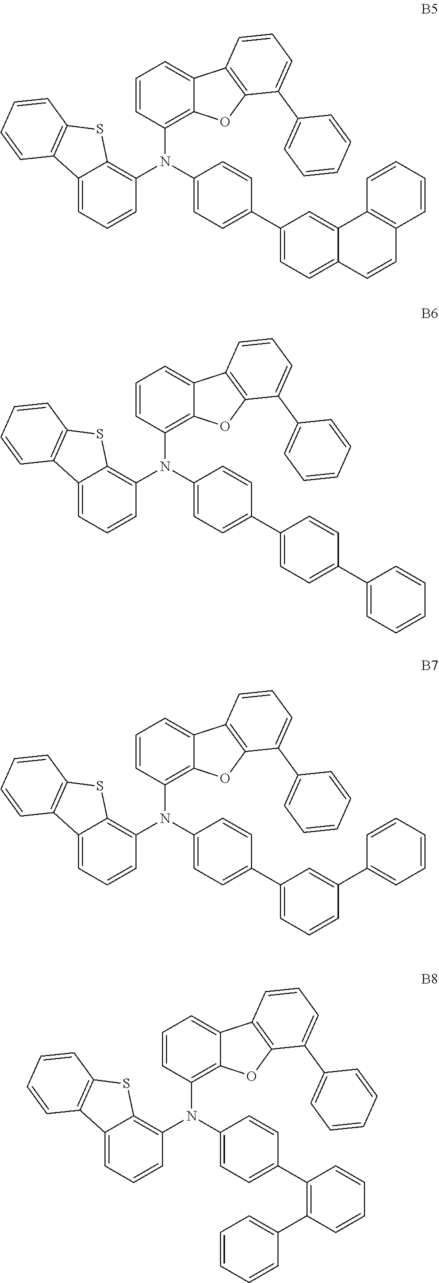 [SMILES: c1ccc(-c2cccc3c2oc2c(N(c4ccc(-c5ccc6ccc7ccccc7c6c5)cc4)c4cccc5c4sc4ccccc45)cccc23)cc1 ; c1ccc(-c2ccc(-c3ccc(N(c4cccc5c4oc4c(-c6ccccc6)cccc45)c4cccc5c4sc4ccccc45)cc3)cc2)cc1 ; c1ccc(-c2cccc(-c3ccc(N(c4cccc5c4oc4c(-c6ccccc6)cccc45)c4cccc5c4sc4ccccc45)cc3)c2)cc1 ; c1ccc(-c2ccccc2-c2ccc(N(c3cccc4c3oc3c(-c5ccccc5)cccc34)c3cccc4c3sc3ccccc34)cc2)cc1]

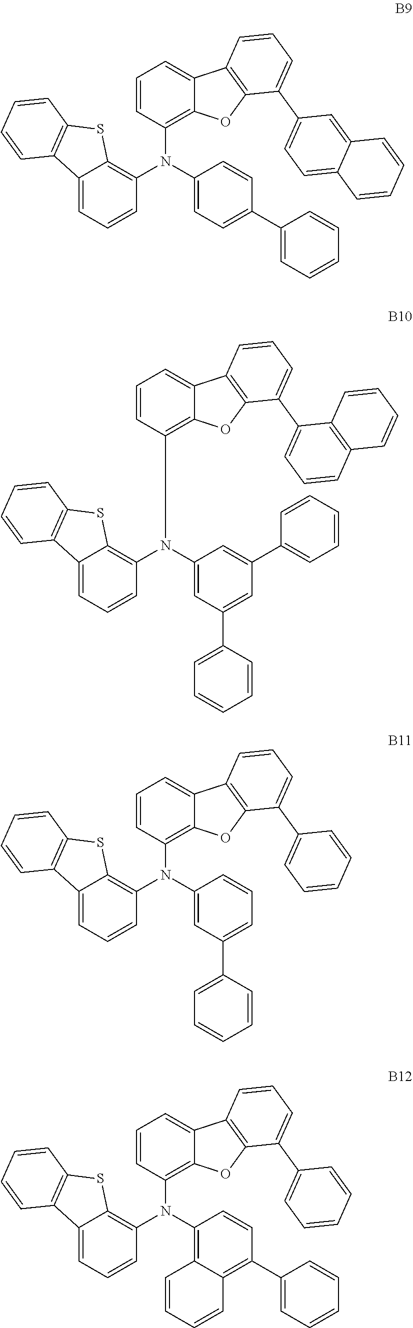

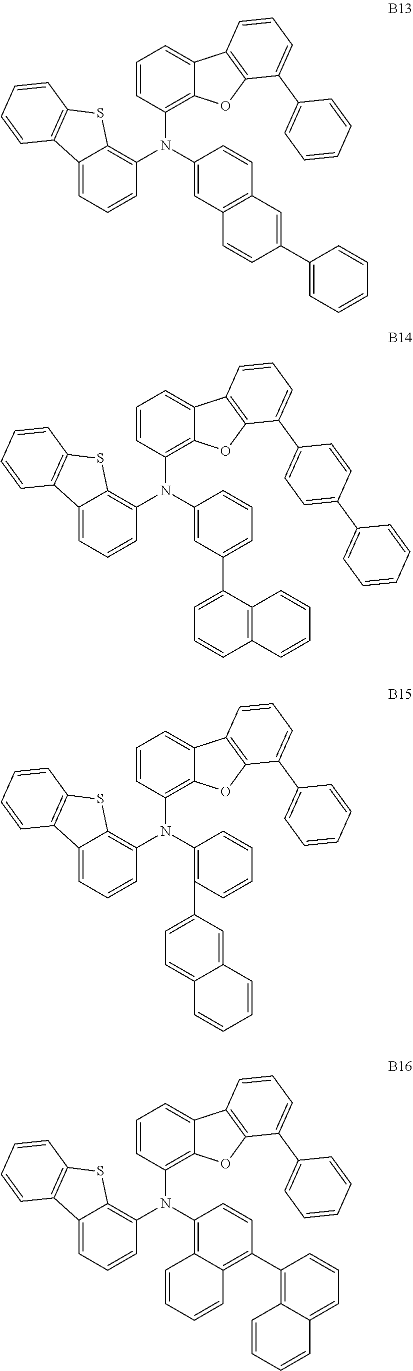 [SMILES: c1ccc(-c2ccc3cc(N(c4cccc5c4oc4c(-c6ccccc6)cccc45)c4cccc5c4sc4ccccc45)ccc3c2)cc1 ; c1ccc(-c2ccc(-c3cccc4c3oc3c(N(c5cccc(-c6cccc7ccccc67)c5)c5cccc6c5sc5ccccc56)cccc34)cc2)cc1 ; c1ccc(-c2cccc3c2oc2c(N(c4ccccc4-c4ccc5ccccc5c4)c4cccc5c4sc4ccccc45)cccc23)cc1 ; c1ccc(-c2cccc3c2oc2c(N(c4ccc(-c5cccc6ccccc56)c5ccccc45)c4cccc5c4sc4ccccc45)cccc23)cc1]

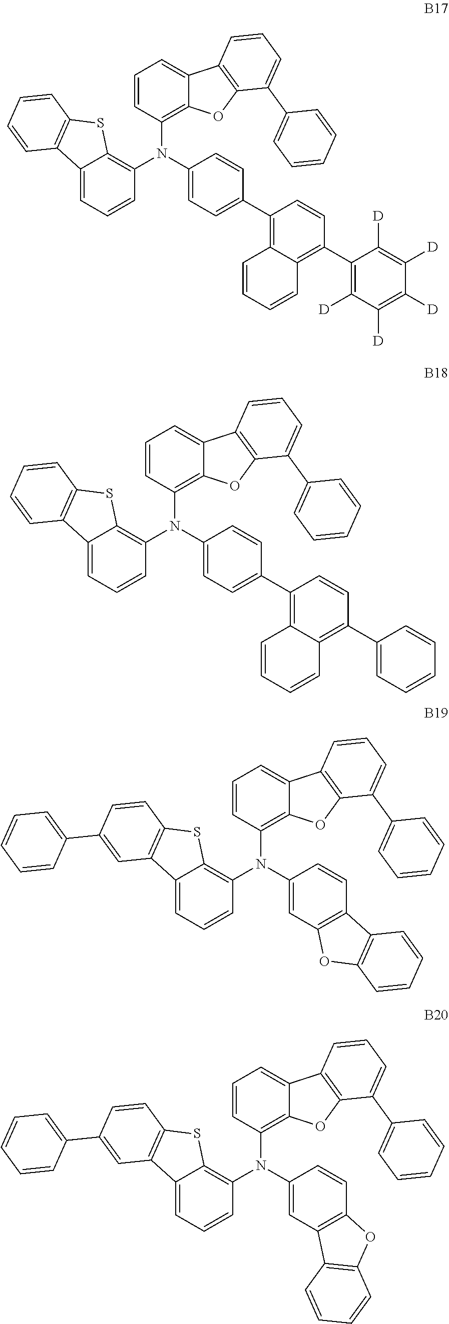 [SMILES: [2H]c1c([2H])c([2H])c(-c2ccc(-c3ccc(N(c4cccc5c4oc4c(-c6ccccc6)cccc45)c4cccc5c4sc4ccccc45)cc3)c3ccccc23)c([2H])c1[2H] ; c1ccc(-c2ccc(-c3ccc(N(c4cccc5c4oc4c(-c6ccccc6)cccc45)c4cccc5c4sc4ccccc45)cc3)c3ccccc23)cc1 ; c1ccc(-c2ccc3sc4c(N(c5ccc6c(c5)oc5ccccc56)c5cccc6c5oc5c(-c7ccccc7)cccc56)cccc4c3c2)cc1 ; c1ccc(-c2ccc3sc4c(N(c5ccc6oc7ccccc7c6c5)c5cccc6c5oc5c(-c7ccccc7)cccc56)cccc4c3c2)cc1]

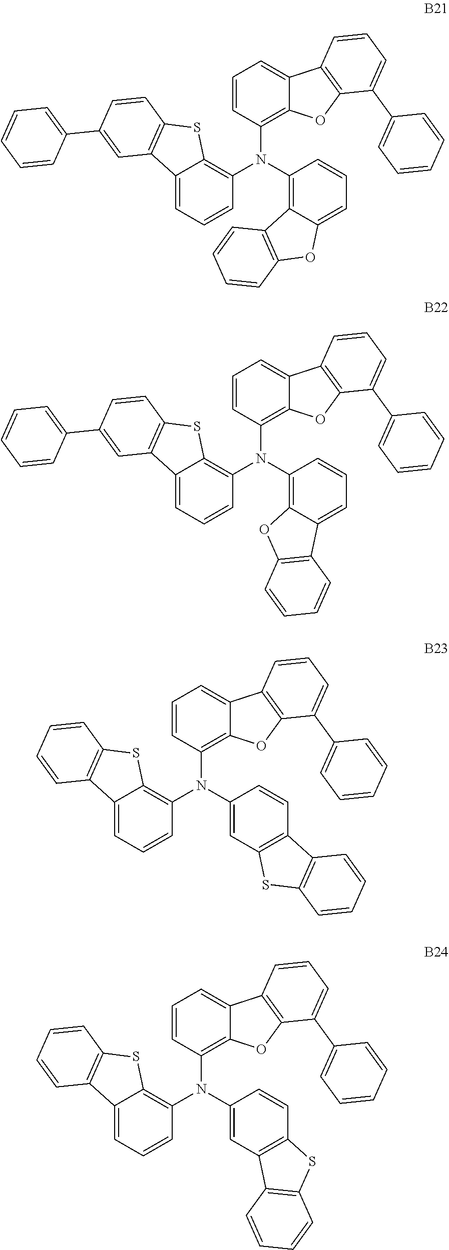 [SMILES: c1ccc(-c2ccc3sc4c(N(c5cccc6c5oc5c(-c7ccccc7)cccc56)c5cccc6oc7ccccc7c56)cccc4c3c2)cc1 ; c1ccc(-c2ccc3sc4c(N(c5cccc6c5oc5ccccc56)c5cccc6c5oc5c(-c7ccccc7)cccc56)cccc4c3c2)cc1 ; c1ccc(-c2cccc3c2oc2c(N(c4ccc5c(c4)sc4ccccc45)c4cccc5c4sc4ccccc45)cccc23)cc1 ; c1ccc(-c2cccc3c2oc2c(N(c4ccc5sc6ccccc6c5c4)c4cccc5c4sc4ccccc45)cccc23)cc1]

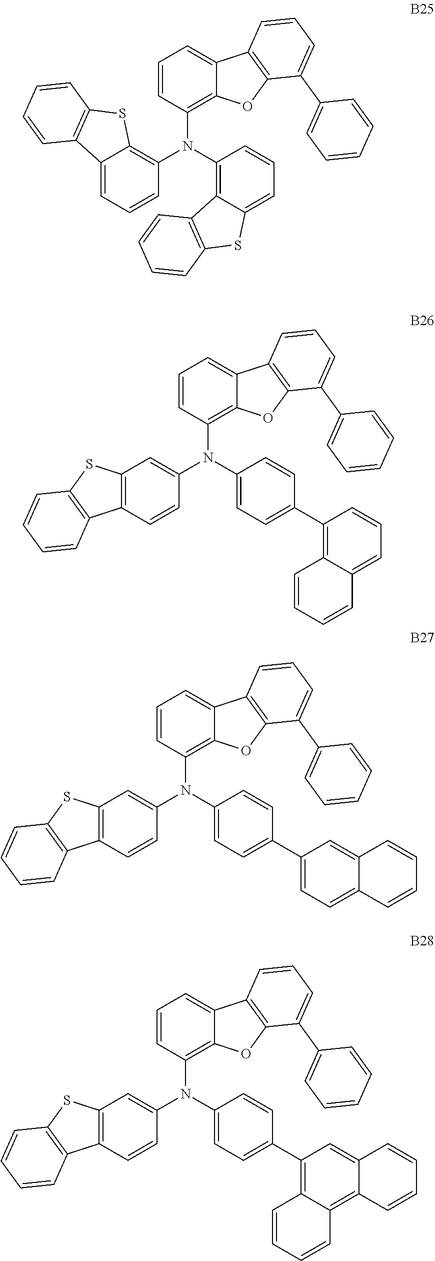 [SMILES: c1ccc(-c2cccc3c2oc2c(N(c4cccc5c4sc4ccccc45)c4cccc5sc6ccccc6c45)cccc23)cc1 ; c1ccc(-c2cccc3c2oc2c(N(c4ccc(-c5cccc6ccccc56)cc4)c4ccc5c(c4)sc4ccccc45)cccc23)cc1 ; c1ccc(-c2cccc3c2oc2c(N(c4ccc(-c5ccc6ccccc6c5)cc4)c4ccc5c(c4)sc4ccccc45)cccc23)cc1 ; c1ccc(-c2cccc3c2oc2c(N(c4ccc(-c5cc6ccccc6c6ccccc56)cc4)c4ccc5c(c4)sc4ccccc45)cccc23)cc1]

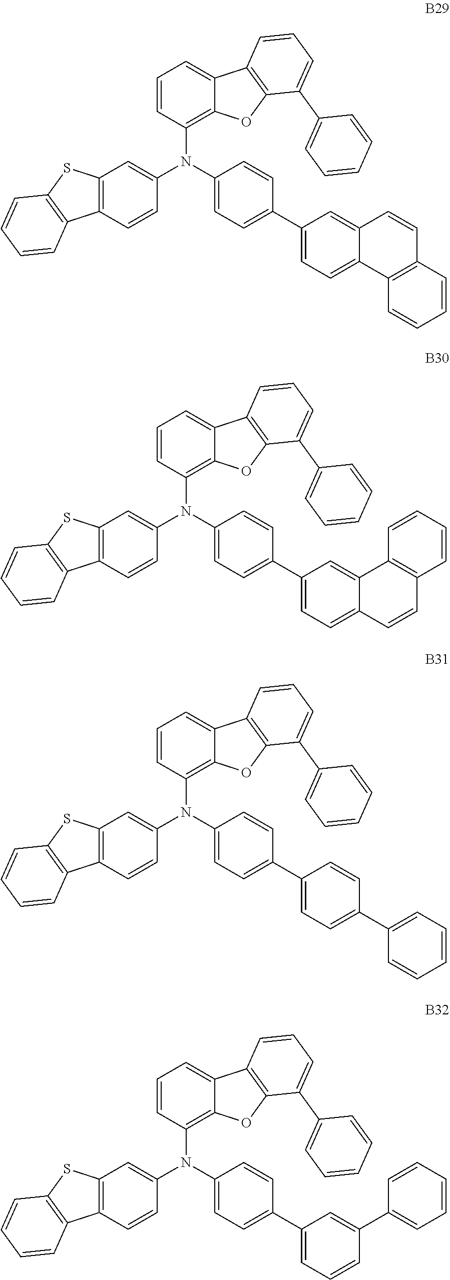 [SMILES: c1ccc(-c2cccc3c2oc2c(N(c4ccc(-c5ccc6c(ccc7ccccc76)c5)cc4)c4ccc5c(c4)sc4ccccc45)cccc23)cc1 ; c1ccc(-c2cccc3c2oc2c(N(c4ccc(-c5ccc6ccc7ccccc7c6c5)cc4)c4ccc5c(c4)sc4ccccc45)cccc23)cc1 ; c1ccc(-c2ccc(-c3ccc(N(c4ccc5c(c4)sc4ccccc45)c4cccc5c4oc4c(-c6ccccc6)cccc45)cc3)cc2)cc1 ; c1ccc(-c2cccc(-c3ccc(N(c4ccc5c(c4)sc4ccccc45)c4cccc5c4oc4c(-c6ccccc6)cccc45)cc3)c2)cc1]

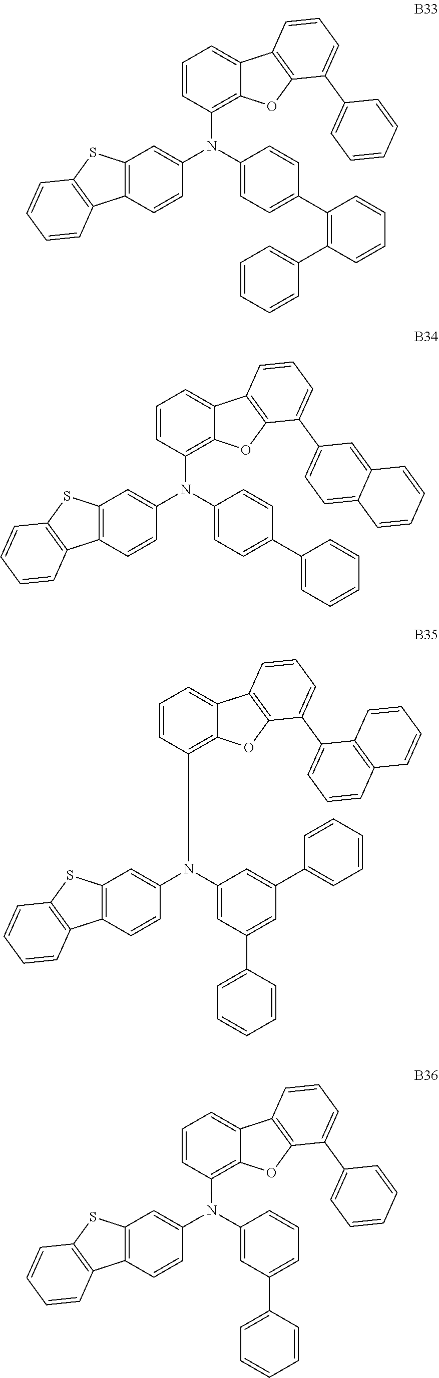 [SMILES: c1ccc(-c2ccccc2-c2ccc(N(c3ccc4c(c3)sc3ccccc34)c3cccc4c3oc3c(-c5ccccc5)cccc34)cc2)cc1 ; c1ccc(-c2ccc(N(c3ccc4c(c3)sc3ccccc34)c3cccc4c3oc3c(-c5ccc6ccccc6c5)cccc34)cc2)cc1 ; c1ccc(-c2cc(-c3ccccc3)cc(N(c3ccc4c(c3)sc3ccccc34)c3cccc4c3oc3c(-c5cccc6ccccc56)cccc34)c2)cc1 ; c1ccc(-c2cccc(N(c3ccc4c(c3)sc3ccccc34)c3cccc4c3oc3c(-c5ccccc5)cccc34)c2)cc1]

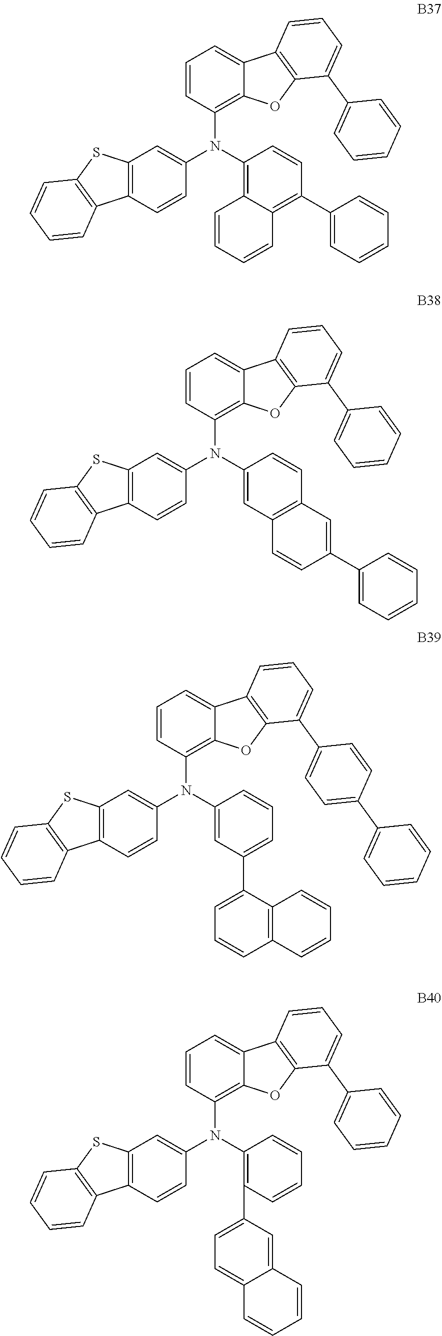 [SMILES: c1ccc(-c2ccc(N(c3ccc4c(c3)sc3ccccc34)c3cccc4c3oc3c(-c5ccccc5)cccc34)c3ccccc23)cc1 ; c1ccc(-c2ccc3cc(N(c4ccc5c(c4)sc4ccccc45)c4cccc5c4oc4c(-c6ccccc6)cccc45)ccc3c2)cc1 ; c1ccc(-c2ccc(-c3cccc4c3oc3c(N(c5cccc(-c6cccc7ccccc67)c5)c5ccc6c(c5)sc5ccccc56)cccc34)cc2)cc1 ; c1ccc(-c2cccc3c2oc2c(N(c4ccc5c(c4)sc4ccccc45)c4ccccc4-c4ccc5ccccc5c4)cccc23)cc1]

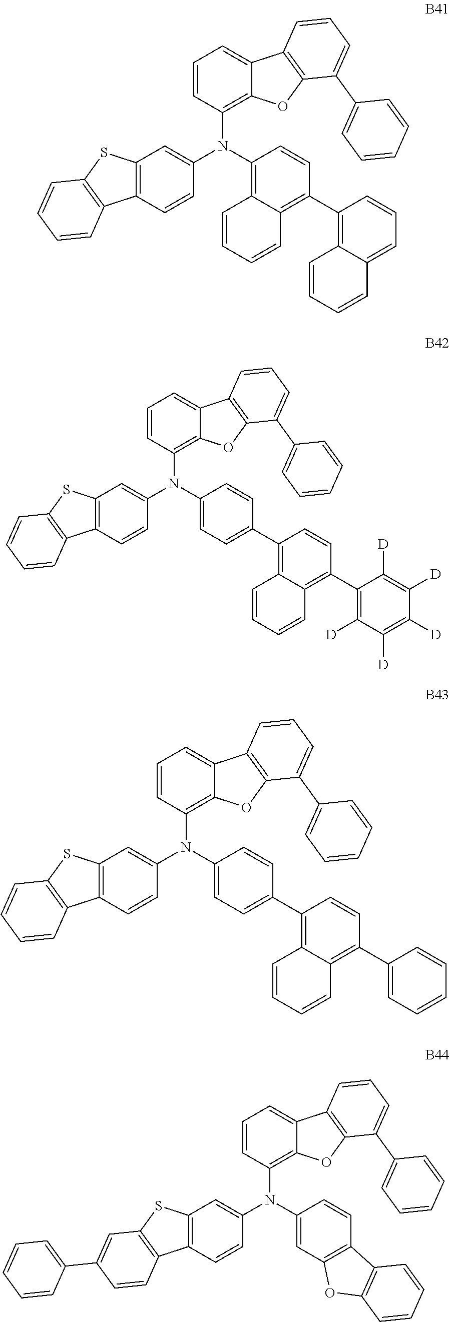 [SMILES: c1ccc(-c2cccc3c2oc2c(N(c4ccc5c(c4)sc4ccccc45)c4ccc(-c5cccc6ccccc56)c5ccccc45)cccc23)cc1 ; [2H]c1c([2H])c([2H])c(-c2ccc(-c3ccc(N(c4ccc5c(c4)sc4ccccc45)c4cccc5c4oc4c(-c6ccccc6)cccc45)cc3)c3ccccc23)c([2H])c1[2H] ; c1ccc(-c2ccc(-c3ccc(N(c4ccc5c(c4)sc4ccccc45)c4cccc5c4oc4c(-c6ccccc6)cccc45)cc3)c3ccccc23)cc1 ; c1ccc(-c2ccc3c(c2)sc2cc(N(c4ccc5c(c4)oc4ccccc45)c4cccc5c4oc4c(-c6ccccc6)cccc45)ccc23)cc1]

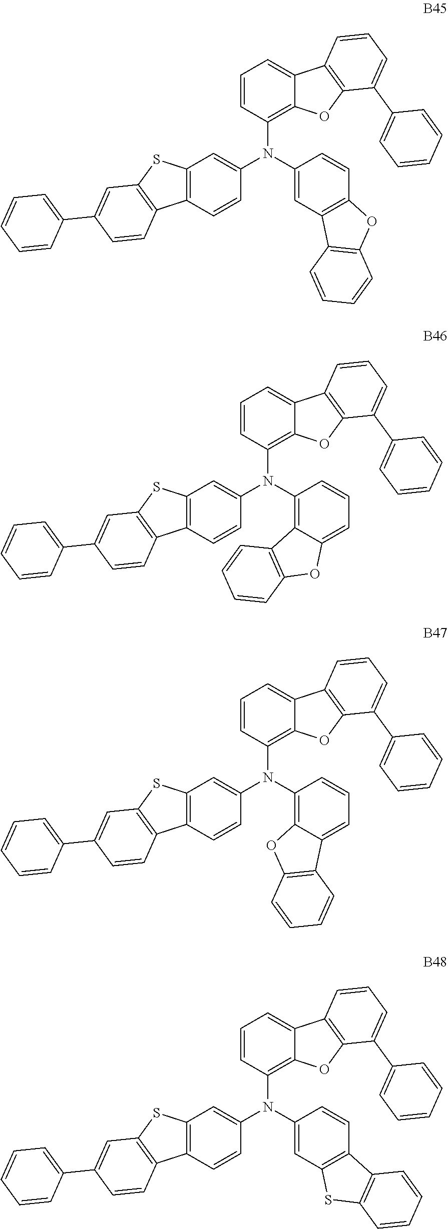 [SMILES: c1ccc(-c2ccc3c(c2)sc2cc(N(c4ccc5oc6ccccc6c5c4)c4cccc5c4oc4c(-c6ccccc6)cccc45)ccc23)cc1 ; c1ccc(-c2ccc3c(c2)sc2cc(N(c4cccc5c4oc4c(-c6ccccc6)cccc45)c4cccc5oc6ccccc6c45)ccc23)cc1 ; c1ccc(-c2ccc3c(c2)sc2cc(N(c4cccc5c4oc4ccccc45)c4cccc5c4oc4c(-c6ccccc6)cccc45)ccc23)cc1 ; c1ccc(-c2ccc3c(c2)sc2cc(N(c4ccc5c(c4)sc4ccccc45)c4cccc5c4oc4c(-c6ccccc6)cccc45)ccc23)cc1]

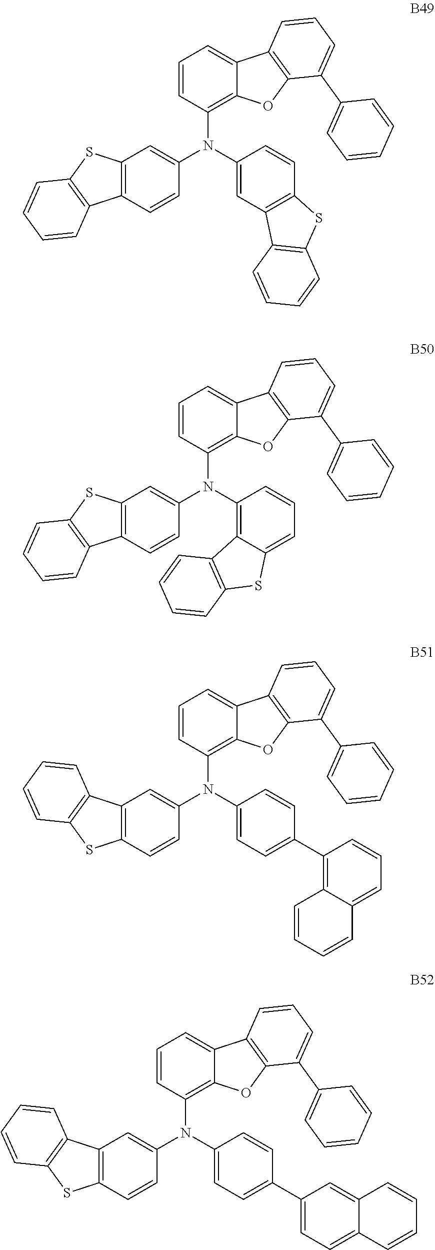 [SMILES: c1ccc(-c2cccc3c2oc2c(N(c4ccc5c(c4)sc4ccccc45)c4ccc5sc6ccccc6c5c4)cccc23)cc1 ; c1ccc(-c2cccc3c2oc2c(N(c4ccc5c(c4)sc4ccccc45)c4cccc5sc6ccccc6c45)cccc23)cc1 ; c1ccc(-c2cccc3c2oc2c(N(c4ccc(-c5cccc6ccccc56)cc4)c4ccc5sc6ccccc6c5c4)cccc23)cc1 ; c1ccc(-c2cccc3c2oc2c(N(c4ccc(-c5ccc6ccccc6c5)cc4)c4ccc5sc6ccccc6c5c4)cccc23)cc1]

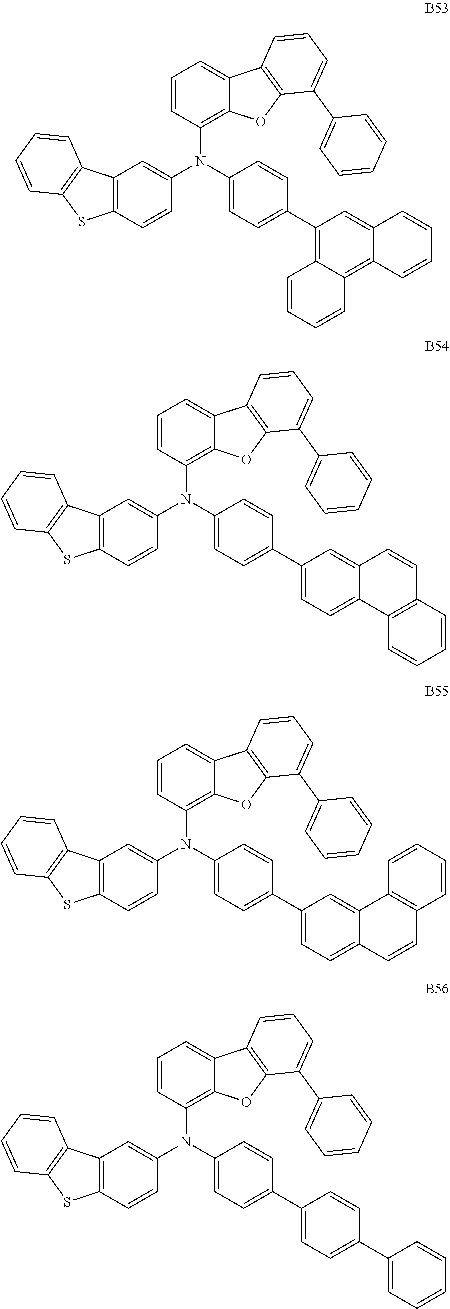 [SMILES: c1ccc(-c2cccc3c2oc2c(N(c4ccc(-c5cc6ccccc6c6ccccc56)cc4)c4ccc5sc6ccccc6c5c4)cccc23)cc1 ; c1ccc(-c2cccc3c2oc2c(N(c4ccc(-c5ccc6c(ccc7ccccc76)c5)cc4)c4ccc5sc6ccccc6c5c4)cccc23)cc1 ; c1ccc(-c2cccc3c2oc2c(N(c4ccc(-c5ccc6ccc7ccccc7c6c5)cc4)c4ccc5sc6ccccc6c5c4)cccc23)cc1 ; c1ccc(-c2ccc(-c3ccc(N(c4ccc5sc6ccccc6c5c4)c4cccc5c4oc4c(-c6ccccc6)cccc45)cc3)cc2)cc1]

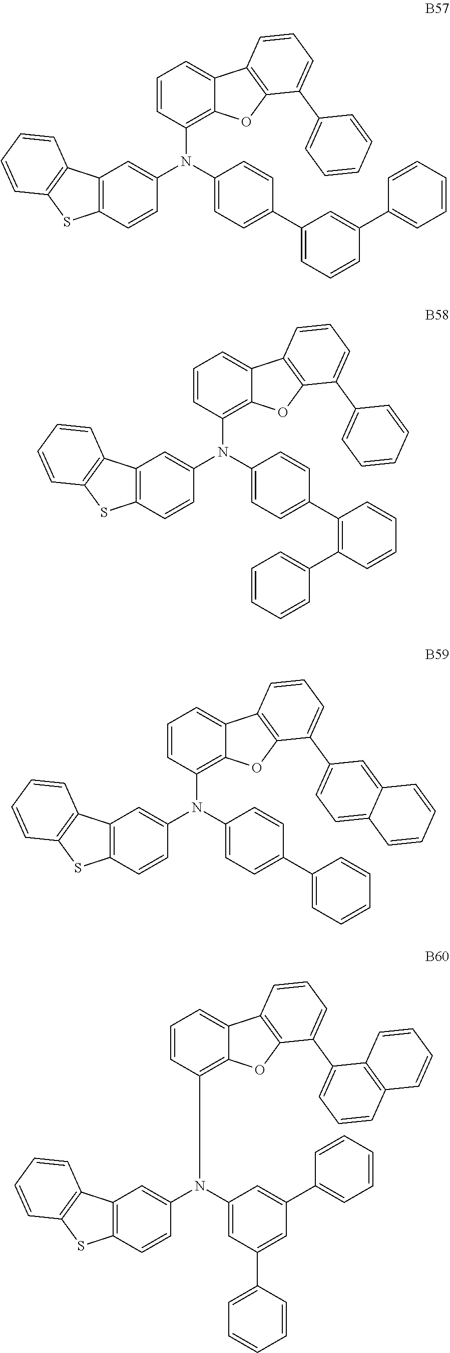

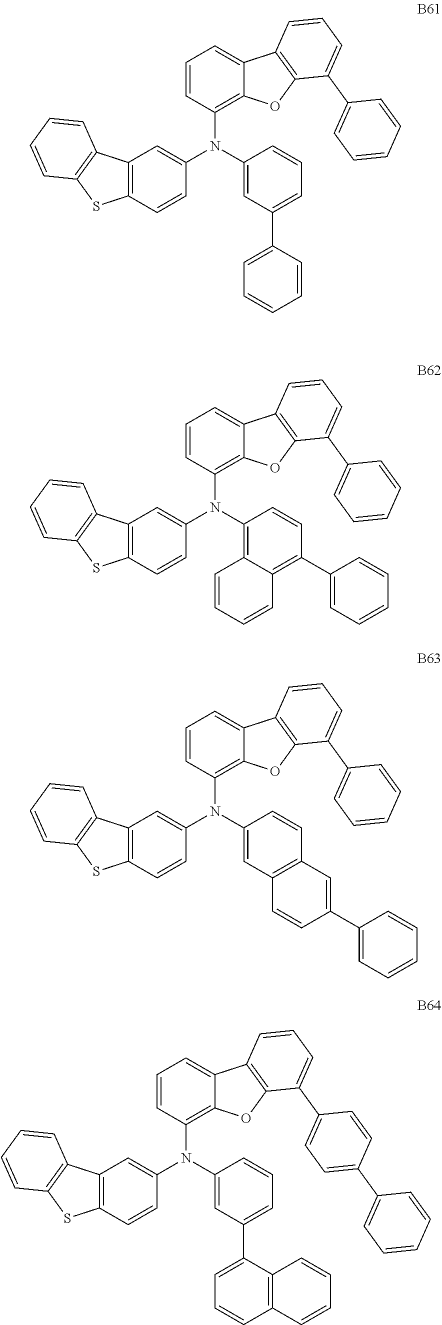 [SMILES: c1ccc(-c2cccc(N(c3ccc4sc5ccccc5c4c3)c3cccc4c3oc3c(-c5ccccc5)cccc34)c2)cc1 ; c1ccc(-c2ccc(N(c3ccc4sc5ccccc5c4c3)c3cccc4c3oc3c(-c5ccccc5)cccc34)c3ccccc23)cc1 ; c1ccc(-c2ccc3cc(N(c4ccc5sc6ccccc6c5c4)c4cccc5c4oc4c(-c6ccccc6)cccc45)ccc3c2)cc1 ; c1ccc(-c2ccc(-c3cccc4c3oc3c(N(c5cccc(-c6cccc7ccccc67)c5)c5ccc6sc7ccccc7c6c5)cccc34)cc2)cc1]

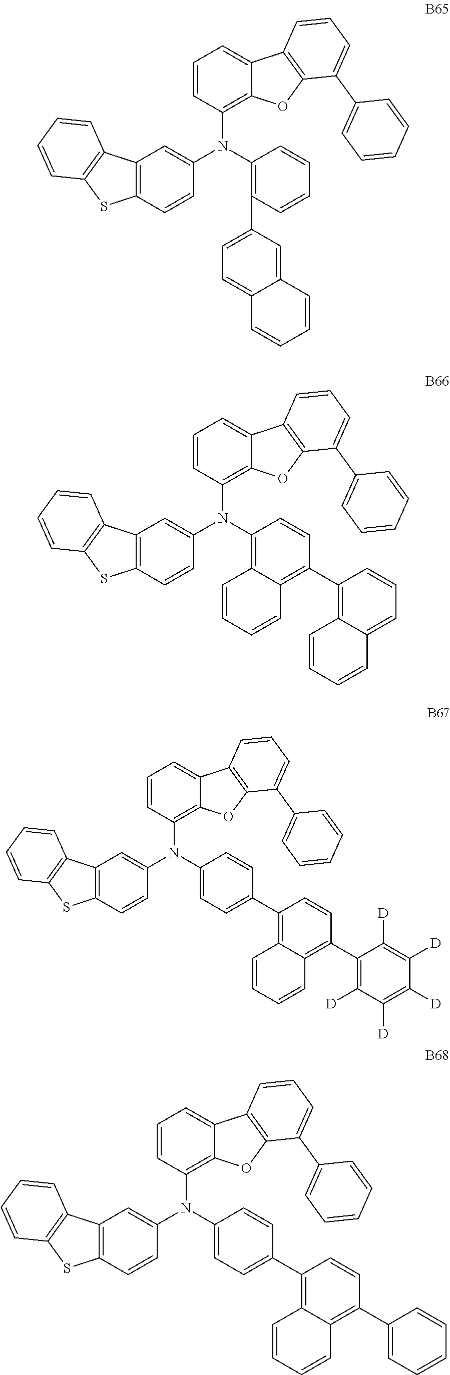 [SMILES: c1ccc(-c2cccc3c2oc2c(N(c4ccc5sc6ccccc6c5c4)c4ccccc4-c4ccc5ccccc5c4)cccc23)cc1 ; c1ccc(-c2cccc3c2oc2c(N(c4ccc5sc6ccccc6c5c4)c4ccc(-c5cccc6ccccc56)c5ccccc45)cccc23)cc1 ; [2H]c1c([2H])c([2H])c(-c2ccc(-c3ccc(N(c4ccc5sc6ccccc6c5c4)c4cccc5c4oc4c(-c6ccccc6)cccc45)cc3)c3ccccc23)c([2H])c1[2H] ; c1ccc(-c2ccc(-c3ccc(N(c4ccc5sc6ccccc6c5c4)c4cccc5c4oc4c(-c6ccccc6)cccc45)cc3)c3ccccc23)cc1]

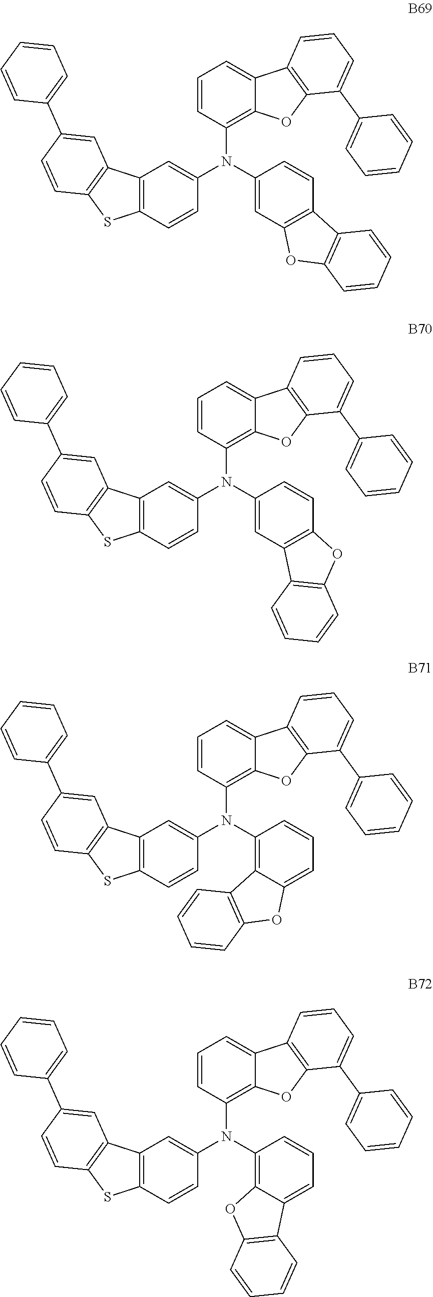 [SMILES: c1ccc(-c2ccc3sc4ccc(N(c5ccc6c(c5)oc5ccccc56)c5cccc6c5oc5c(-c7ccccc7)cccc56)cc4c3c2)cc1 ; c1ccc(-c2ccc3sc4ccc(N(c5ccc6oc7ccccc7c6c5)c5cccc6c5oc5c(-c7ccccc7)cccc56)cc4c3c2)cc1 ; c1ccc(-c2ccc3sc4ccc(N(c5cccc6c5oc5c(-c7ccccc7)cccc56)c5cccc6oc7ccccc7c56)cc4c3c2)cc1 ; c1ccc(-c2ccc3sc4ccc(N(c5cccc6c5oc5ccccc56)c5cccc6c5oc5c(-c7ccccc7)cccc56)cc4c3c2)cc1]

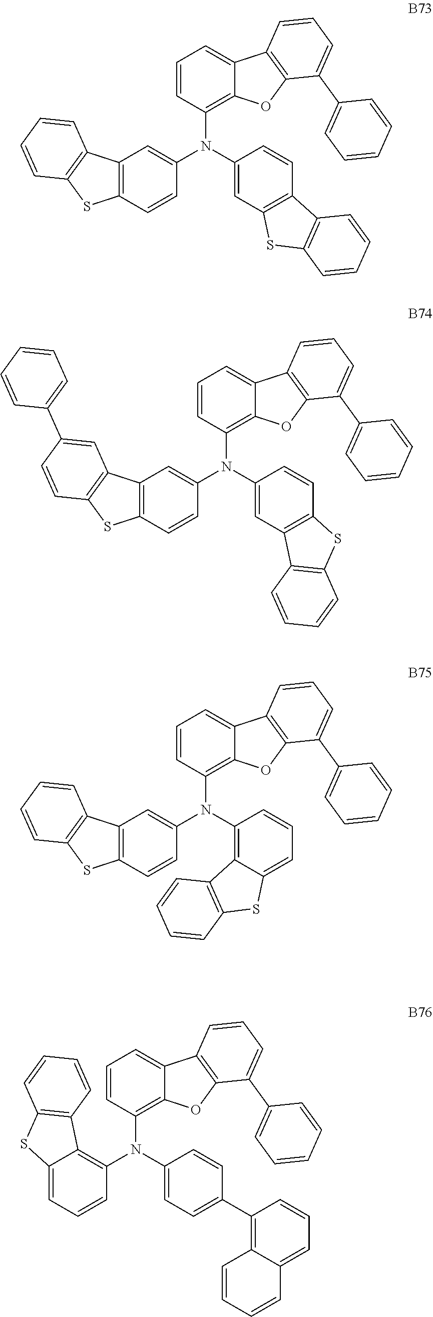 [SMILES: c1ccc(-c2cccc3c2oc2c(N(c4ccc5c(c4)sc4ccccc45)c4ccc5sc6ccccc6c5c4)cccc23)cc1 ; c1ccc(-c2ccc3sc4ccc(N(c5ccc6sc7ccccc7c6c5)c5cccc6c5oc5c(-c7ccccc7)cccc56)cc4c3c2)cc1 ; c1ccc(-c2cccc3c2oc2c(N(c4ccc5sc6ccccc6c5c4)c4cccc5sc6ccccc6c45)cccc23)cc1 ; c1ccc(-c2cccc3c2oc2c(N(c4ccc(-c5cccc6ccccc56)cc4)c4cccc5sc6ccccc6c45)cccc23)cc1]

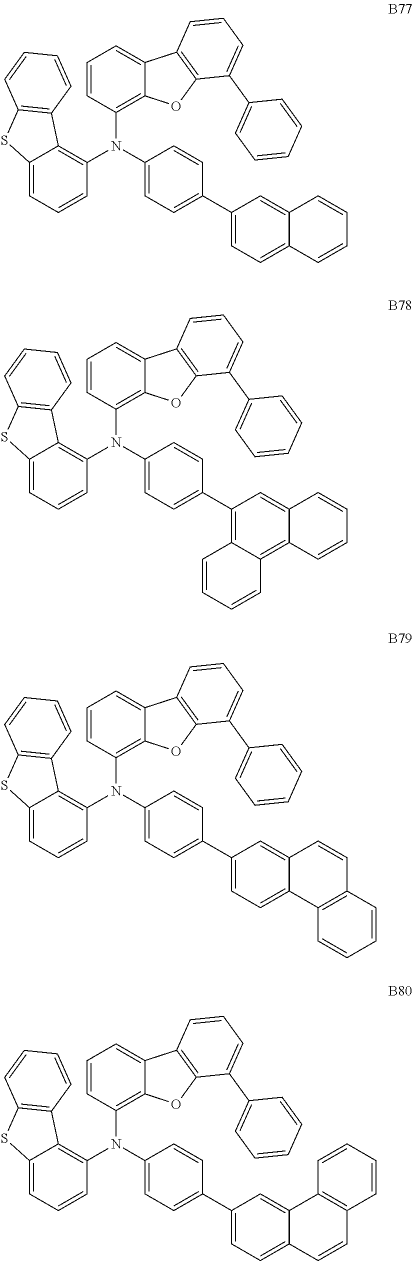 [SMILES: c1ccc(-c2cccc3c2oc2c(N(c4ccc(-c5ccc6ccccc6c5)cc4)c4cccc5sc6ccccc6c45)cccc23)cc1 ; c1ccc(-c2cccc3c2oc2c(N(c4ccc(-c5cc6ccccc6c6ccccc56)cc4)c4cccc5sc6ccccc6c45)cccc23)cc1 ; c1ccc(-c2cccc3c2oc2c(N(c4ccc(-c5ccc6c(ccc7ccccc76)c5)cc4)c4cccc5sc6ccccc6c45)cccc23)cc1 ; c1ccc(-c2cccc3c2oc2c(N(c4ccc(-c5ccc6ccc7ccccc7c6c5)cc4)c4cccc5sc6ccccc6c45)cccc23)cc1]

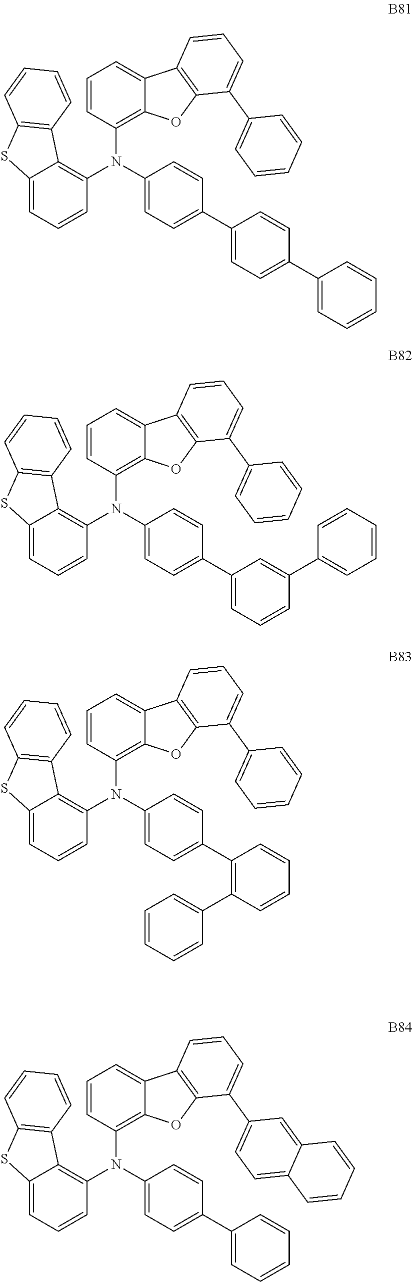 [SMILES: c1ccc(-c2ccc(-c3ccc(N(c4cccc5c4oc4c(-c6ccccc6)cccc45)c4cccc5sc6ccccc6c45)cc3)cc2)cc1 ; c1ccc(-c2cccc(-c3ccc(N(c4cccc5c4oc4c(-c6ccccc6)cccc45)c4cccc5sc6ccccc6c45)cc3)c2)cc1 ; c1ccc(-c2ccccc2-c2ccc(N(c3cccc4c3oc3c(-c5ccccc5)cccc34)c3cccc4sc5ccccc5c34)cc2)cc1 ; c1ccc(-c2ccc(N(c3cccc4c3oc3c(-c5ccc6ccccc6c5)cccc34)c3cccc4sc5ccccc5c34)cc2)cc1]

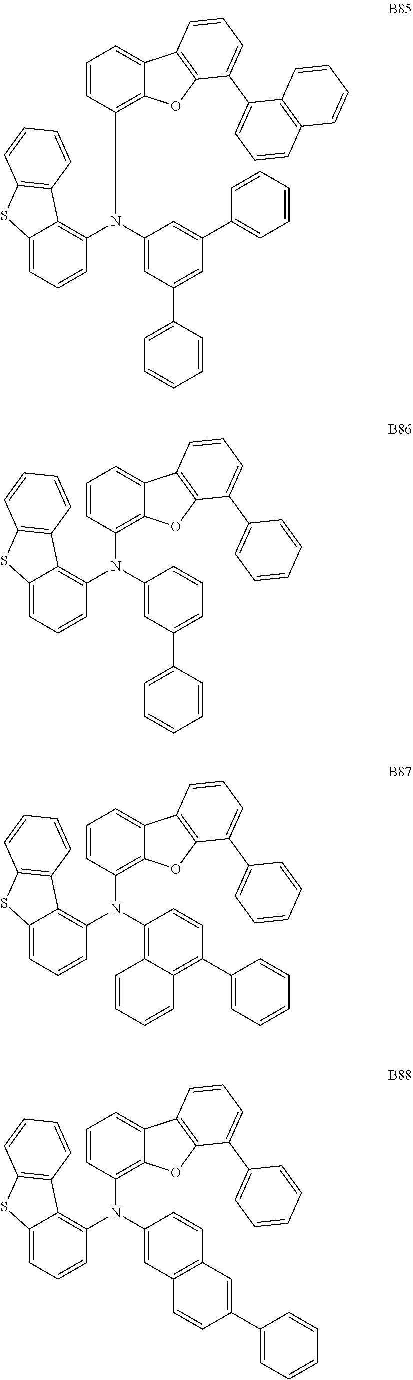 [SMILES: c1ccc(-c2cc(-c3ccccc3)cc(N(c3cccc4c3oc3c(-c5cccc6ccccc56)cccc34)c3cccc4sc5ccccc5c34)c2)cc1 ; c1ccc(-c2cccc(N(c3cccc4c3oc3c(-c5ccccc5)cccc34)c3cccc4sc5ccccc5c34)c2)cc1 ; c1ccc(-c2ccc(N(c3cccc4c3oc3c(-c5ccccc5)cccc34)c3cccc4sc5ccccc5c34)c3ccccc23)cc1 ; c1ccc(-c2ccc3cc(N(c4cccc5c4oc4c(-c6ccccc6)cccc45)c4cccc5sc6ccccc6c45)ccc3c2)cc1]

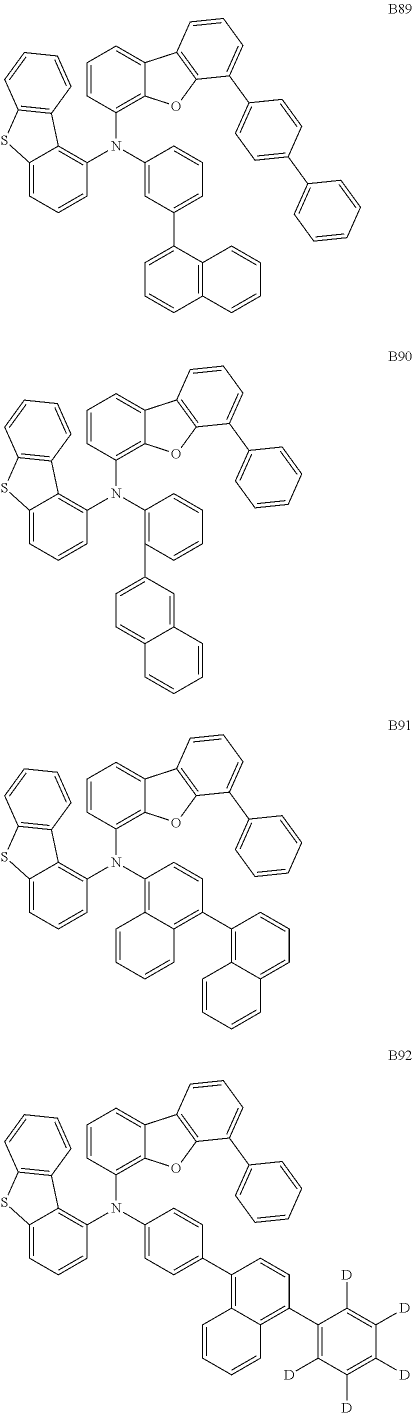 [SMILES: c1ccc(-c2ccc(-c3cccc4c3oc3c(N(c5cccc(-c6cccc7ccccc67)c5)c5cccc6sc7ccccc7c56)cccc34)cc2)cc1 ; c1ccc(-c2cccc3c2oc2c(N(c4ccccc4-c4ccc5ccccc5c4)c4cccc5sc6ccccc6c45)cccc23)cc1 ; c1ccc(-c2cccc3c2oc2c(N(c4ccc(-c5cccc6ccccc56)c5ccccc45)c4cccc5sc6ccccc6c45)cccc23)cc1 ; [2H]c1c([2H])c([2H])c(-c2ccc(-c3ccc(N(c4cccc5c4oc4c(-c6ccccc6)cccc45)c4cccc5sc6ccccc6c45)cc3)c3ccccc23)c([2H])c1[2H]]

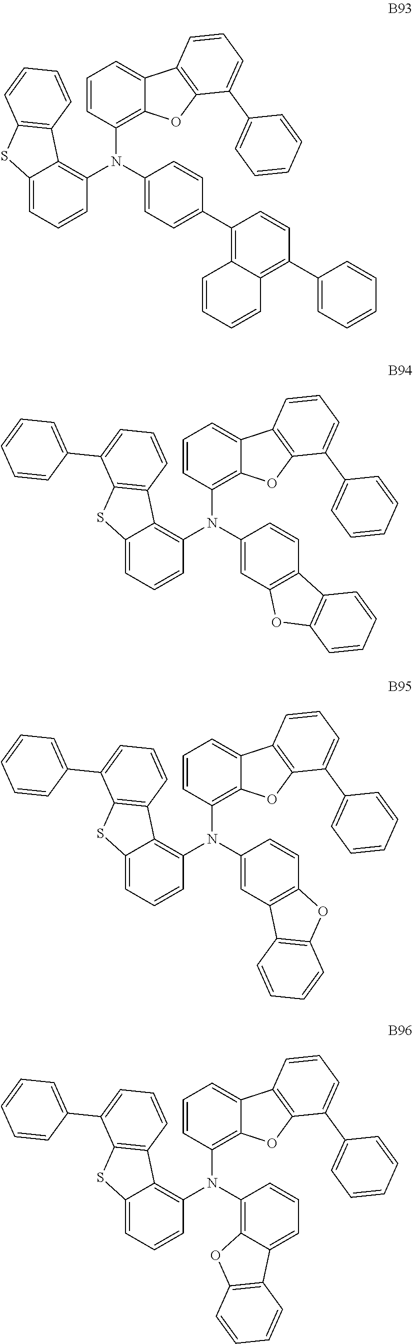 [SMILES: c1ccc(-c2ccc(-c3ccc(N(c4cccc5c4oc4c(-c6ccccc6)cccc45)c4cccc5sc6ccccc6c45)cc3)c3ccccc23)cc1 ; c1ccc(-c2cccc3c2oc2c(N(c4ccc5c(c4)oc4ccccc45)c4cccc5sc6c(-c7ccccc7)cccc6c45)cccc23)cc1 ; c1ccc(-c2cccc3c2oc2c(N(c4ccc5oc6ccccc6c5c4)c4cccc5sc6c(-c7ccccc7)cccc6c45)cccc23)cc1 ; c1ccc(-c2cccc3c2oc2c(N(c4cccc5c4oc4ccccc45)c4cccc5sc6c(-c7ccccc7)cccc6c45)cccc23)cc1]

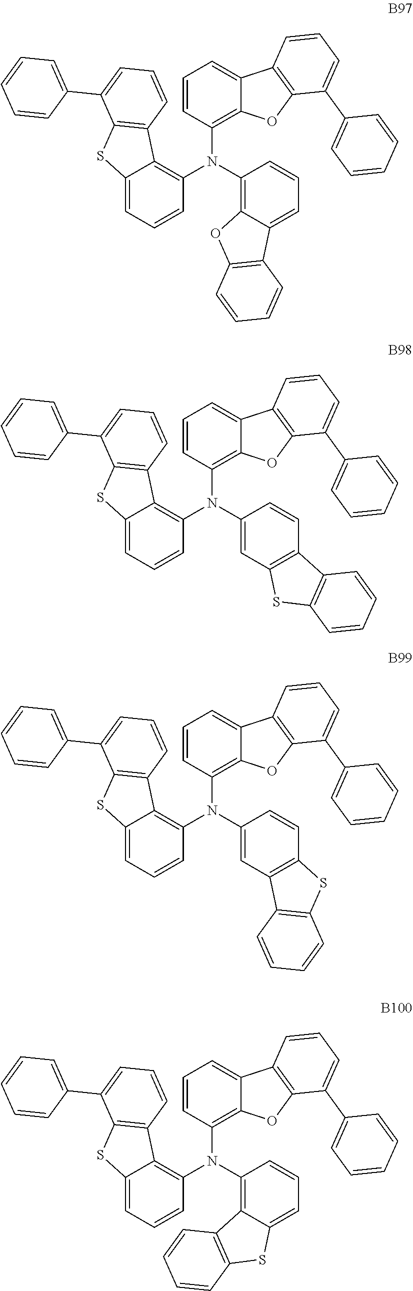 [SMILES: c1ccc(-c2cccc3c2oc2c(N(c4cccc5c4oc4ccccc45)c4cccc5sc6c(-c7ccccc7)cccc6c45)cccc23)cc1 ; c1ccc(-c2cccc3c2oc2c(N(c4ccc5c(c4)sc4ccccc45)c4cccc5sc6c(-c7ccccc7)cccc6c45)cccc23)cc1 ; c1ccc(-c2cccc3c2oc2c(N(c4ccc5sc6ccccc6c5c4)c4cccc5sc6c(-c7ccccc7)cccc6c45)cccc23)cc1 ; c1ccc(-c2cccc3c2oc2c(N(c4cccc5sc6ccccc6c45)c4cccc5sc6c(-c7ccccc7)cccc6c45)cccc23)cc1]

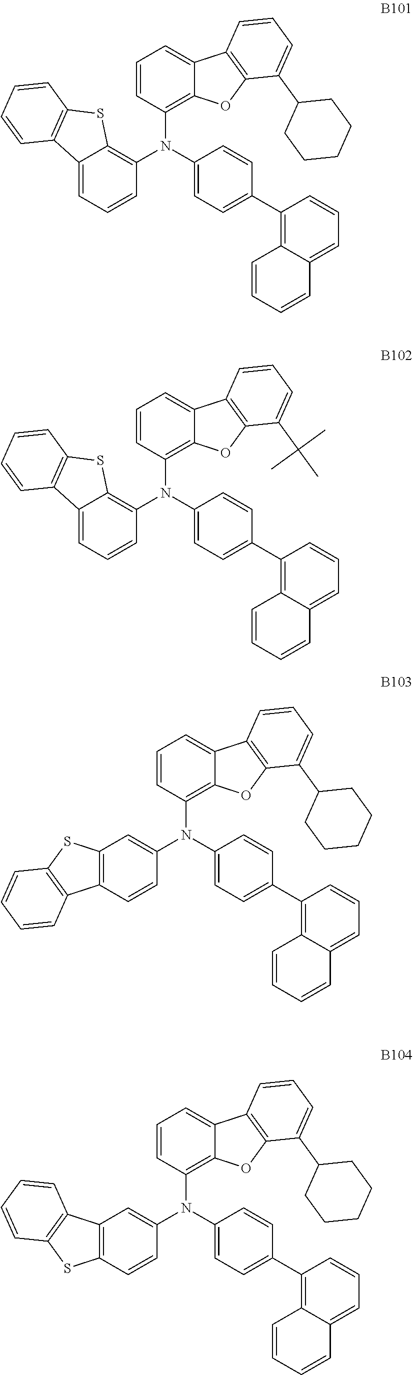 [SMILES: c1ccc2c(-c3ccc(N(c4cccc5c4oc4c(C6CCCCC6)cccc45)c4cccc5c4sc4ccccc45)cc3)cccc2c1 ; CC(C)(C)c1cccc2c1oc1c(N(c3ccc(-c4cccc5ccccc45)cc3)c3cccc4c3sc3ccccc34)cccc12 ; c1ccc2c(-c3ccc(N(c4ccc5c(c4)sc4ccccc45)c4cccc5c4oc4c(C6CCCCC6)cccc45)cc3)cccc2c1 ; c1ccc2c(-c3ccc(N(c4ccc5sc6ccccc6c5c4)c4cccc5c4oc4c(C6CCCCC6)cccc45)cc3)cccc2c1]

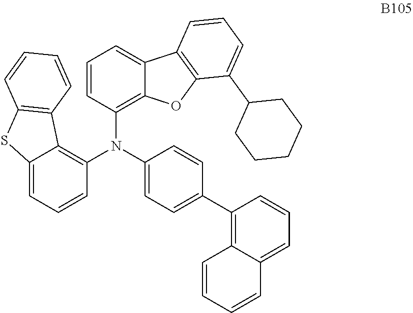 [SMILES: c1ccc2c(-c3ccc(N(c4cccc5c4oc4c(C6CCCCC6)cccc45)c4cccc5sc6ccccc6c45)cc3)cccc2c1]

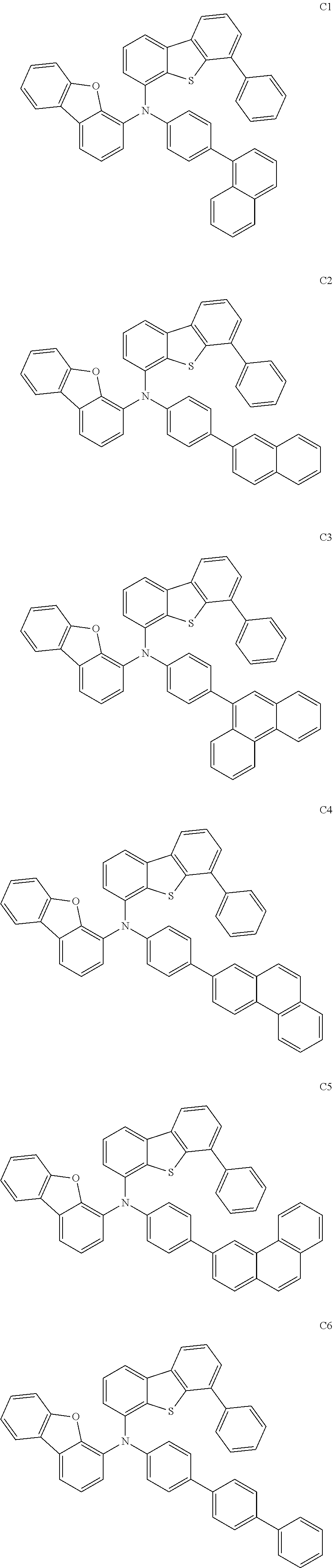

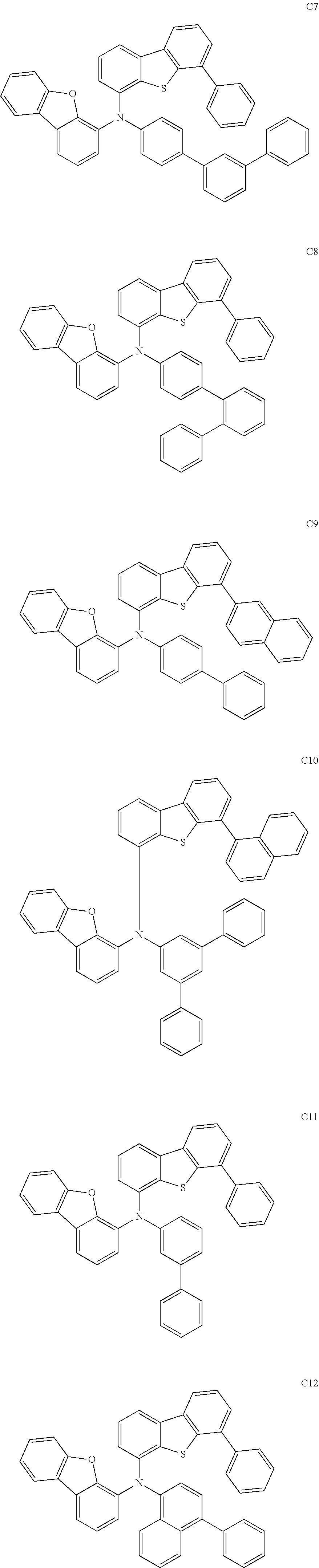

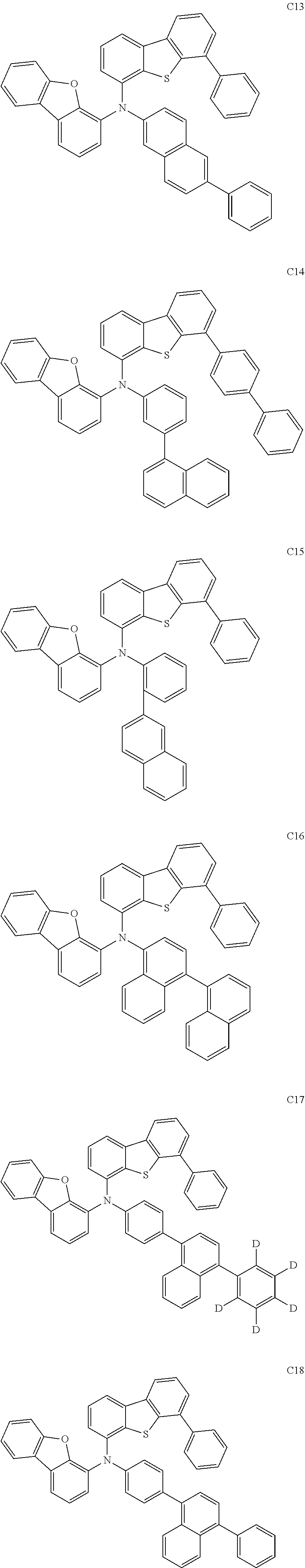

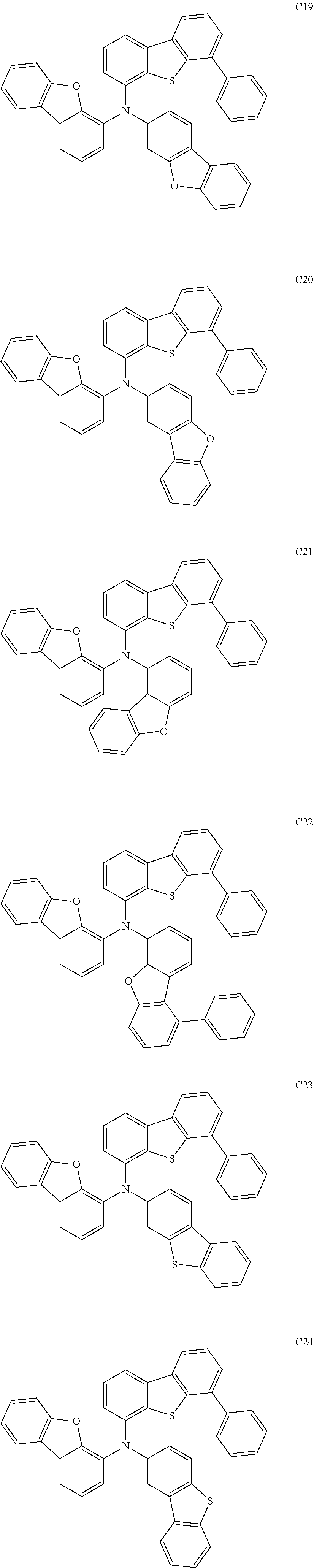 [SMILES: c1ccc(-c2cccc3c2sc2c(N(c4ccc5c(c4)oc4ccccc45)c4cccc5c4oc4ccccc45)cccc23)cc1 ; c1ccc(-c2cccc3c2sc2c(N(c4ccc5oc6ccccc6c5c4)c4cccc5c4oc4ccccc45)cccc23)cc1 ; c1ccc(-c2cccc3c2sc2c(N(c4cccc5c4oc4ccccc45)c4cccc5oc6ccccc6c45)cccc23)cc1 ; c1ccc(-c2cccc3c2sc2c(N(c4cccc5c4oc4ccccc45)c4cccc5c4oc4cccc(-c6ccccc6)c45)cccc23)cc1 ; c1ccc(-c2cccc3c2sc2c(N(c4ccc5c(c4)sc4ccccc45)c4cccc5c4oc4ccccc45)cccc23)cc1 ; c1ccc(-c2cccc3c2sc2c(N(c4ccc5sc6ccccc6c5c4)c4cccc5c4oc4ccccc45)cccc23)cc1]

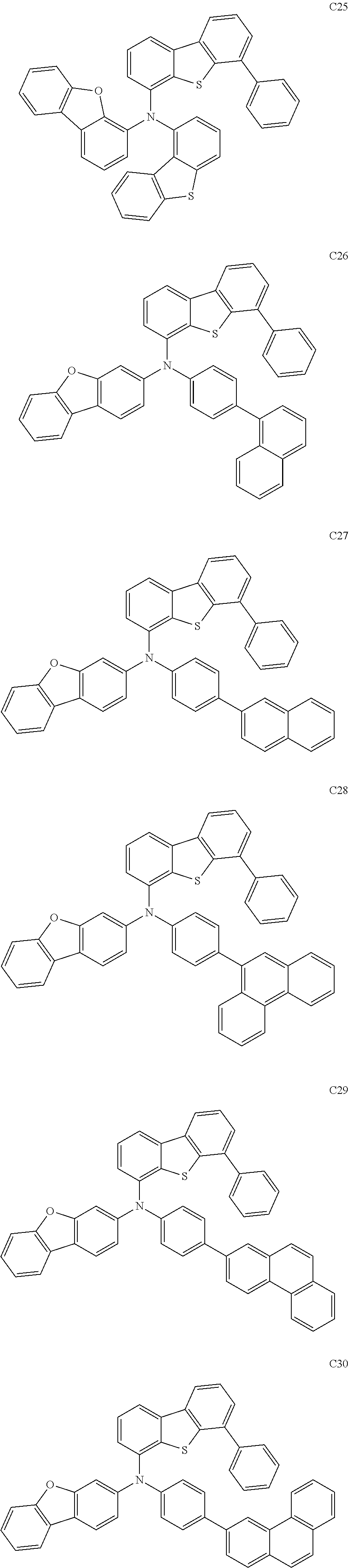

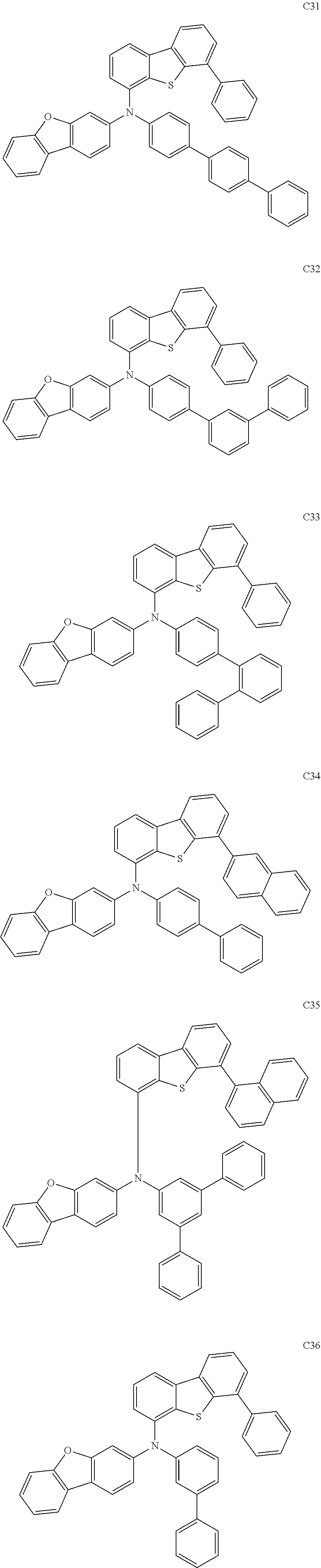

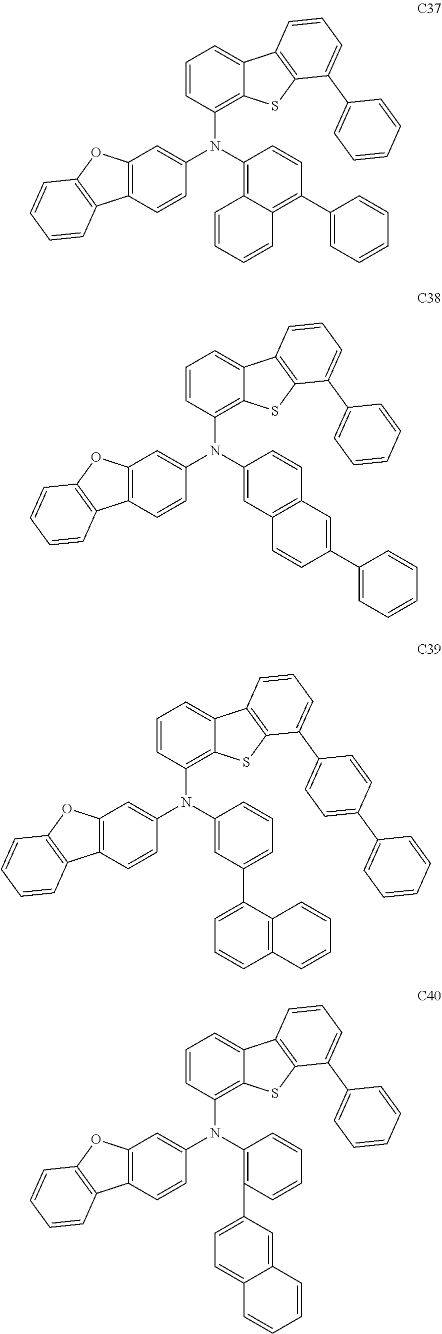

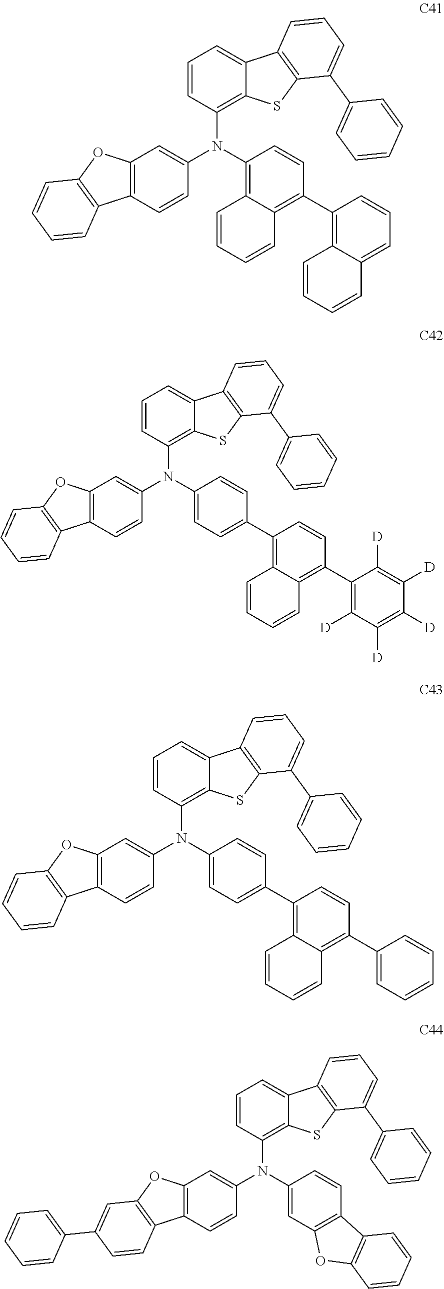 [SMILES: c1ccc(-c2cccc3c2sc2c(N(c4ccc5c(c4)oc4ccccc45)c4ccc(-c5cccc6ccccc56)c5ccccc45)cccc23)cc1 ; [2H]c1c([2H])c([2H])c(-c2ccc(-c3ccc(N(c4ccc5c(c4)oc4ccccc45)c4cccc5c4sc4c(-c6ccccc6)cccc45)cc3)c3ccccc23)c([2H])c1[2H] ; c1ccc(-c2ccc(-c3ccc(N(c4ccc5c(c4)oc4ccccc45)c4cccc5c4sc4c(-c6ccccc6)cccc45)cc3)c3ccccc23)cc1 ; c1ccc(-c2ccc3c(c2)oc2cc(N(c4ccc5c(c4)oc4ccccc45)c4cccc5c4sc4c(-c6ccccc6)cccc45)ccc23)cc1]

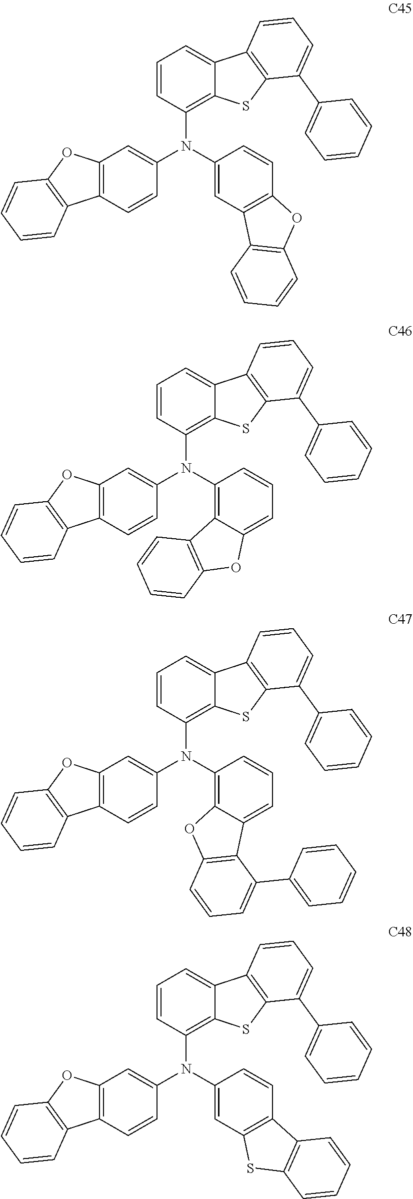

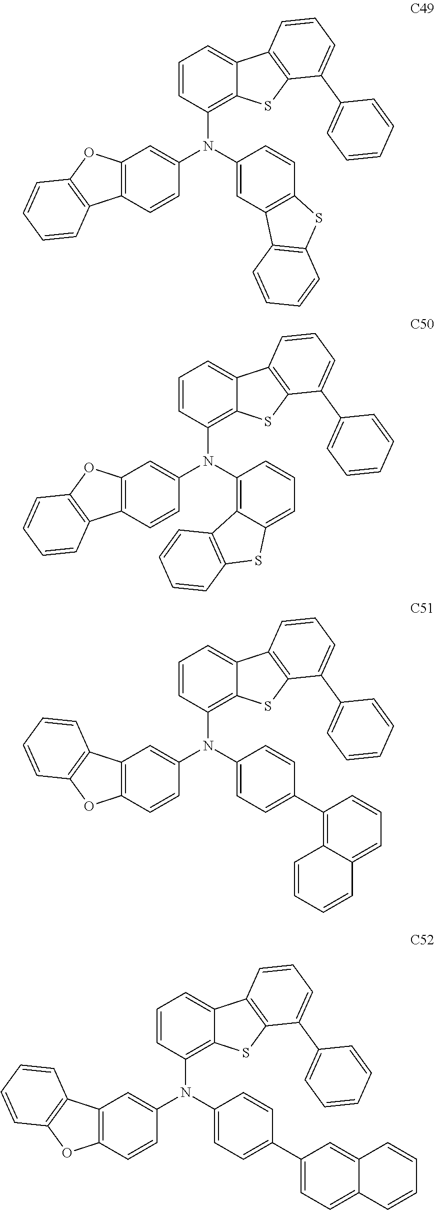

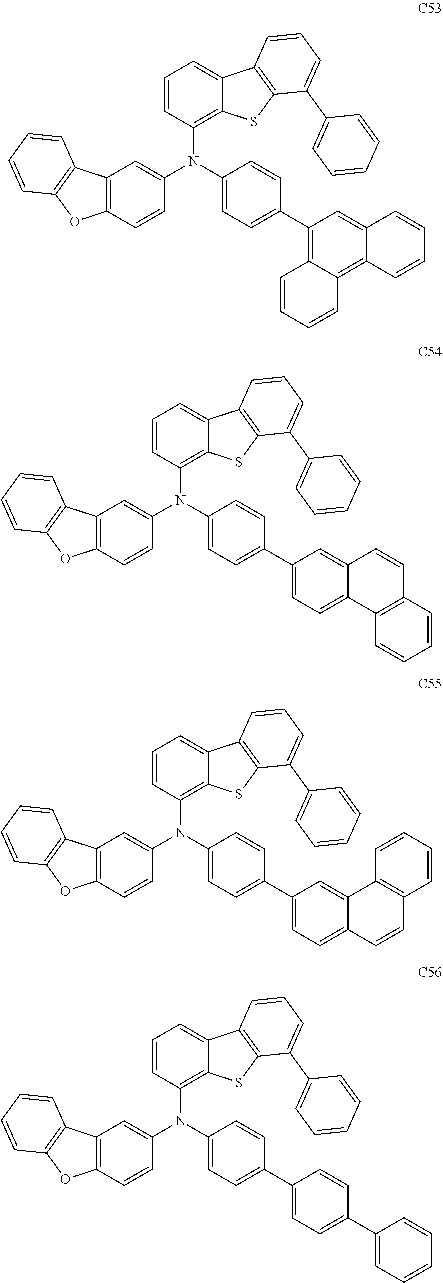 [SMILES: c1ccc(-c2cccc3c2sc2c(N(c4ccc(-c5cc6ccccc6c6ccccc56)cc4)c4ccc5oc6ccccc6c5c4)cccc23)cc1 ; c1ccc(-c2cccc3c2sc2c(N(c4ccc(-c5ccc6c(ccc7ccccc76)c5)cc4)c4ccc5oc6ccccc6c5c4)cccc23)cc1 ; c1ccc(-c2cccc3c2sc2c(N(c4ccc(-c5ccc6ccc7ccccc7c6c5)cc4)c4ccc5oc6ccccc6c5c4)cccc23)cc1 ; c1ccc(-c2ccc(-c3ccc(N(c4ccc5oc6ccccc6c5c4)c4cccc5c4sc4c(-c6ccccc6)cccc45)cc3)cc2)cc1]

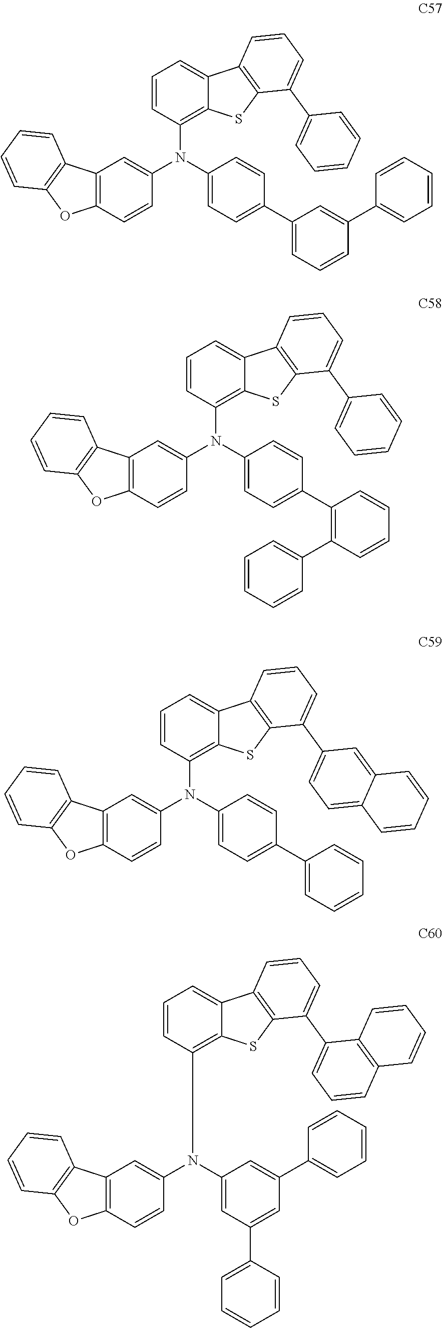

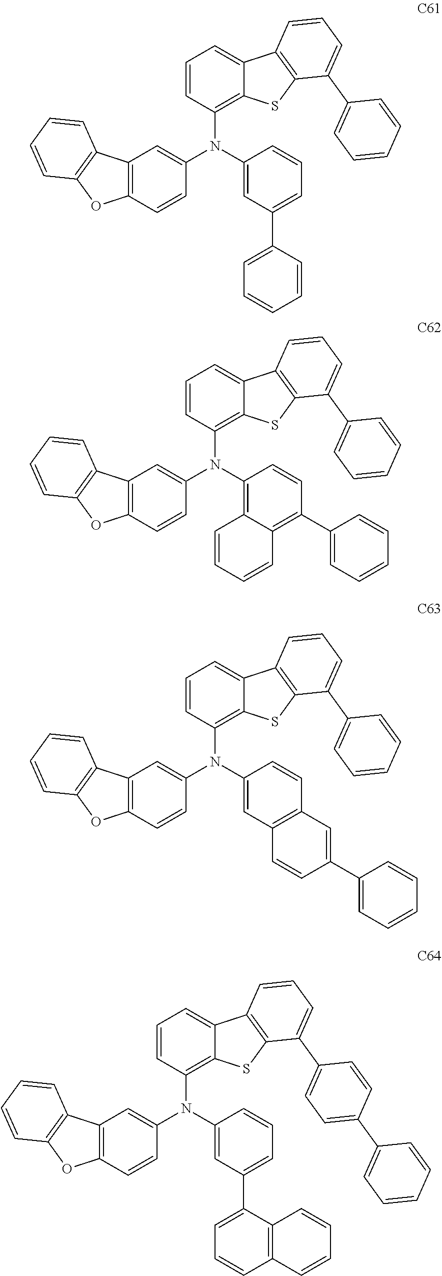

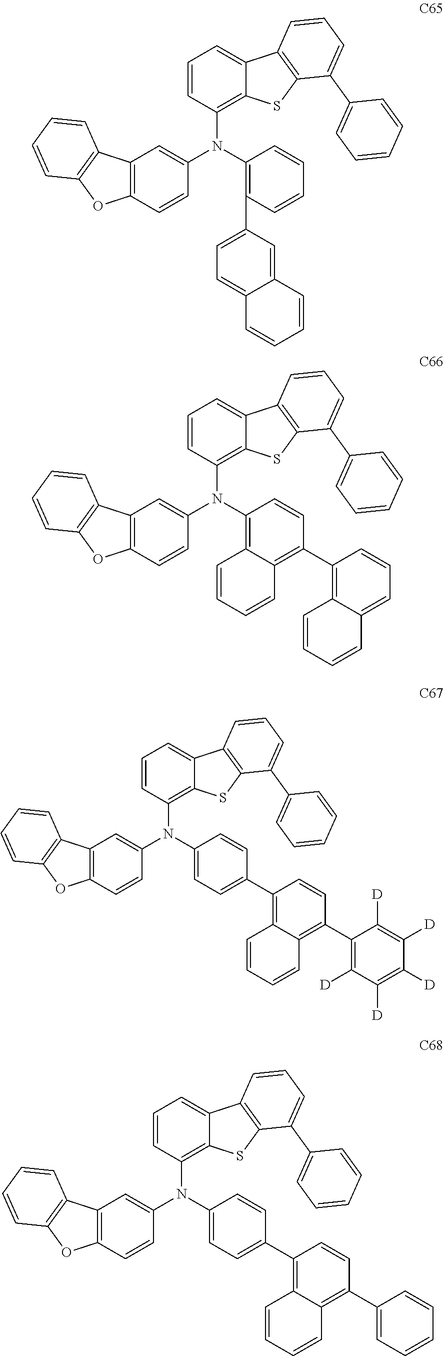

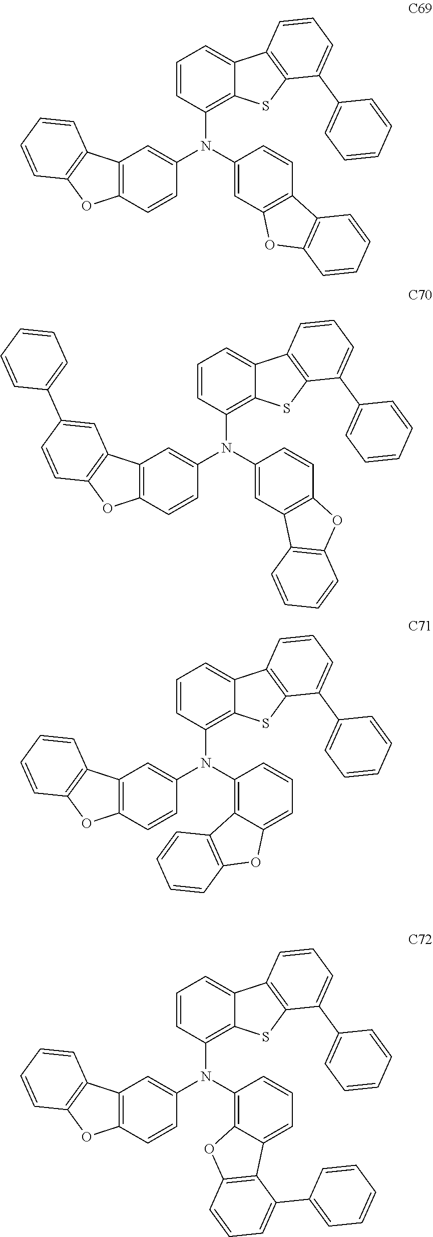

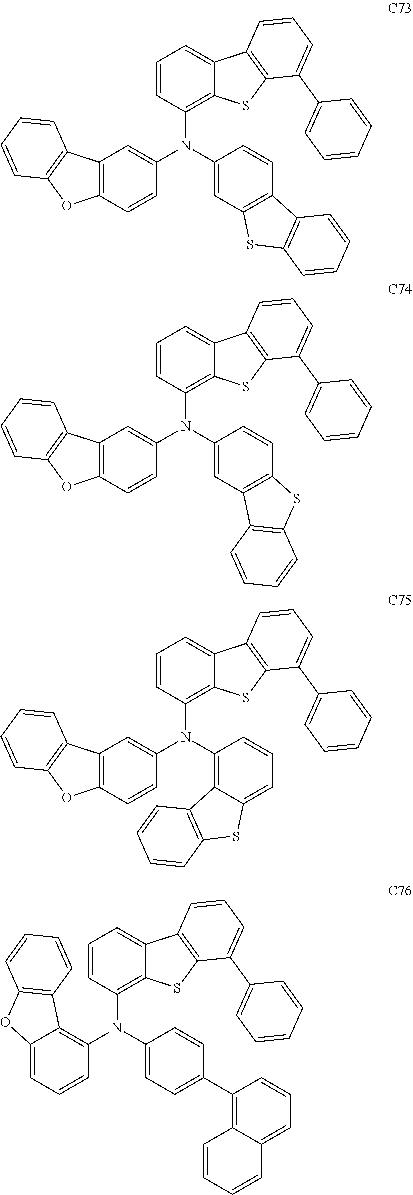

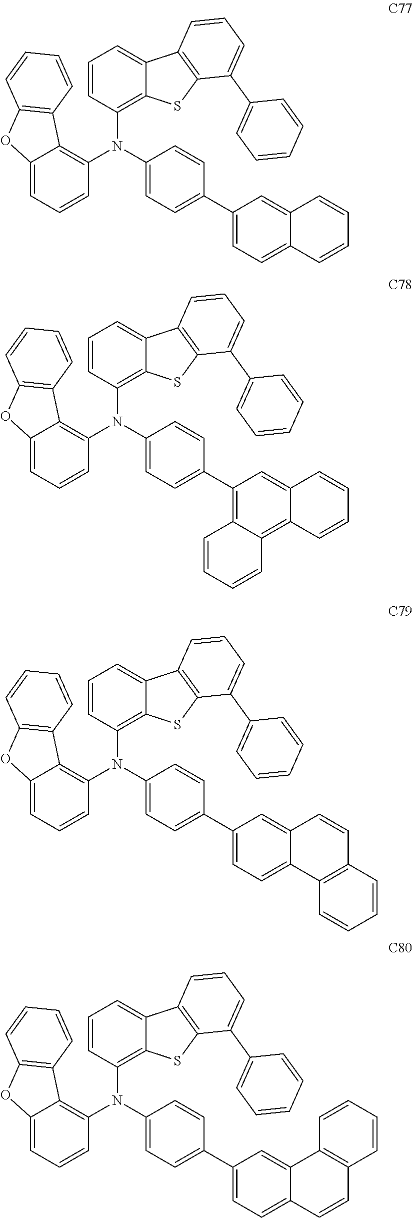 [SMILES: c1ccc(-c2cccc3c2sc2c(N(c4ccc(-c5ccc6ccccc6c5)cc4)c4cccc5oc6ccccc6c45)cccc23)cc1 ; c1ccc(-c2cccc3c2sc2c(N(c4ccc(-c5cc6ccccc6c6ccccc56)cc4)c4cccc5oc6ccccc6c45)cccc23)cc1 ; c1ccc(-c2cccc3c2sc2c(N(c4ccc(-c5ccc6c(ccc7ccccc76)c5)cc4)c4cccc5oc6ccccc6c45)cccc23)cc1 ; c1ccc(-c2cccc3c2sc2c(N(c4ccc(-c5ccc6ccc7ccccc7c6c5)cc4)c4cccc5oc6ccccc6c45)cccc23)cc1]

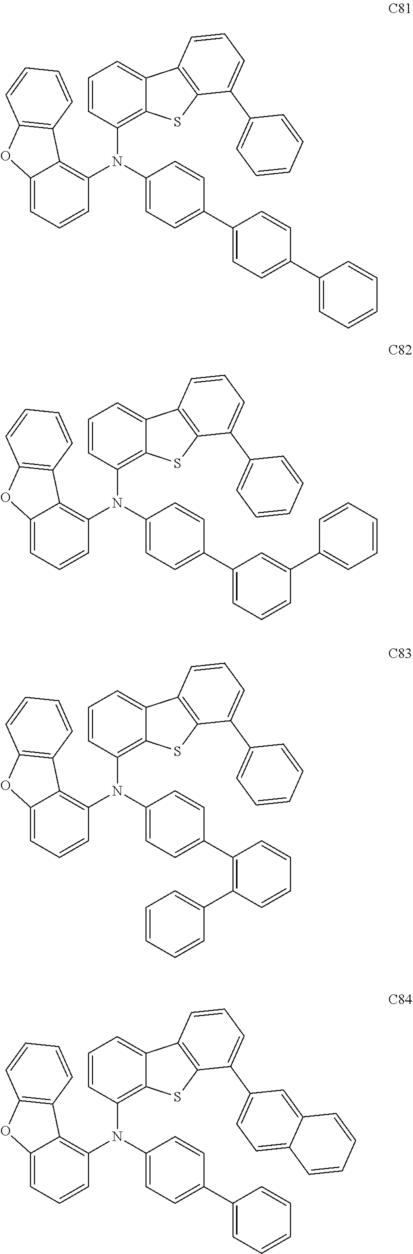 [SMILES: c1ccc(-c2ccc(-c3ccc(N(c4cccc5c4sc4c(-c6ccccc6)cccc45)c4cccc5oc6ccccc6c45)cc3)cc2)cc1 ; c1ccc(-c2cccc(-c3ccc(N(c4cccc5c4sc4c(-c6ccccc6)cccc45)c4cccc5oc6ccccc6c45)cc3)c2)cc1 ; c1ccc(-c2ccccc2-c2ccc(N(c3cccc4c3sc3c(-c5ccccc5)cccc34)c3cccc4oc5ccccc5c34)cc2)cc1 ; c1ccc(-c2ccc(N(c3cccc4c3sc3c(-c5ccc6ccccc6c5)cccc34)c3cccc4oc5ccccc5c34)cc2)cc1]

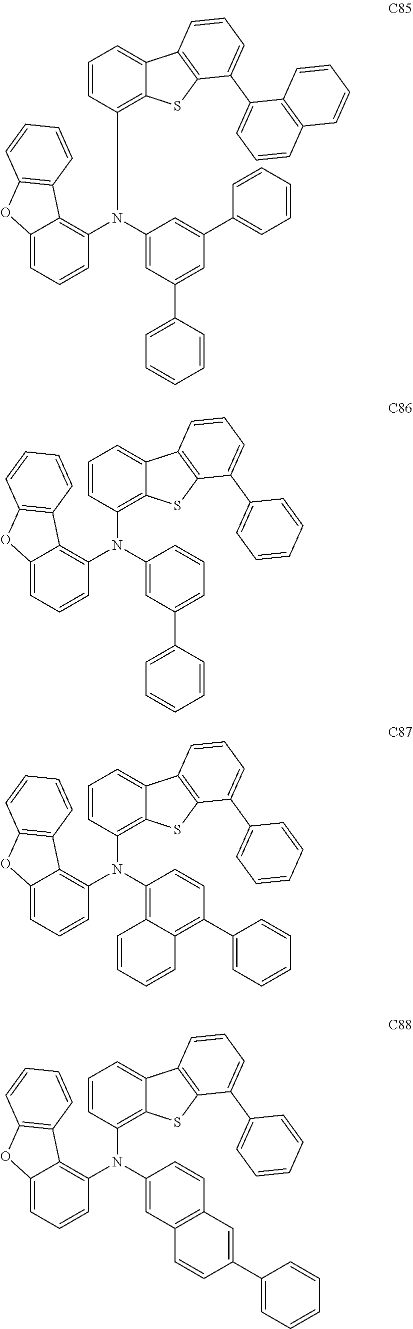

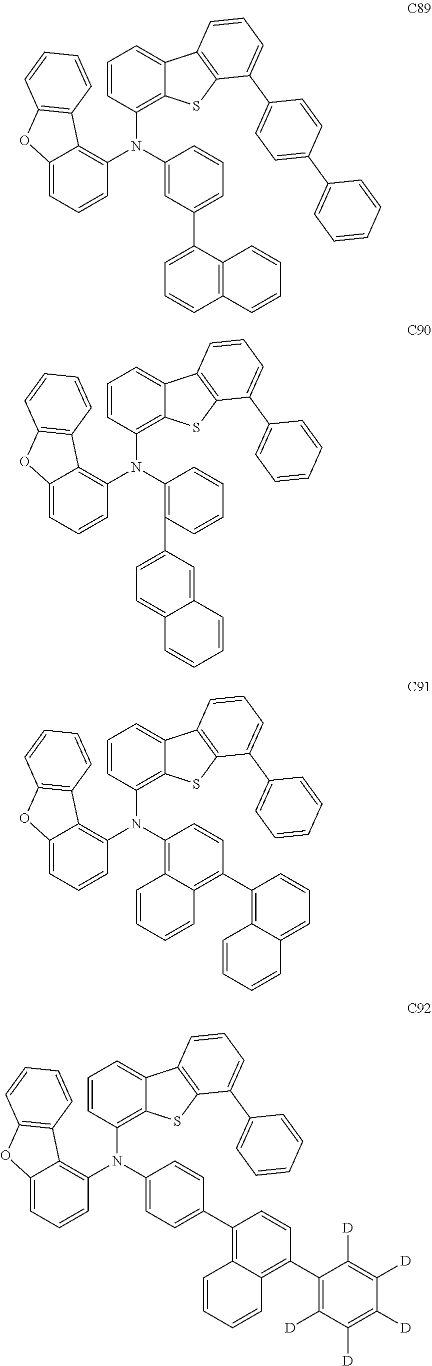

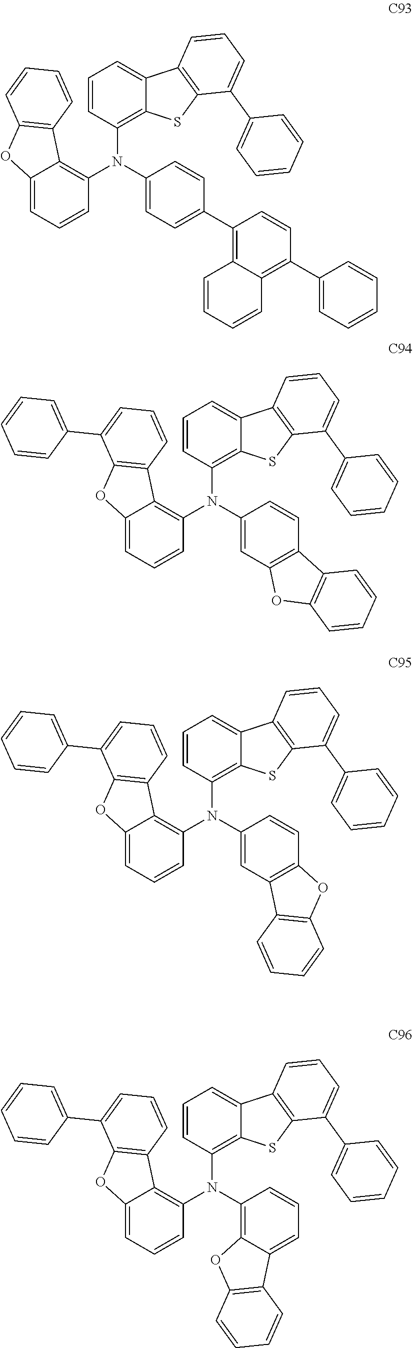 [SMILES: c1ccc(-c2ccc(-c3ccc(N(c4cccc5c4sc4c(-c6ccccc6)cccc45)c4cccc5oc6ccccc6c45)cc3)c3ccccc23)cc1 ; c1ccc(-c2cccc3c2oc2cccc(N(c4ccc5c(c4)oc4ccccc45)c4cccc5c4sc4c(-c6ccccc6)cccc45)c23)cc1 ; c1ccc(-c2cccc3c2oc2cccc(N(c4ccc5oc6ccccc6c5c4)c4cccc5c4sc4c(-c6ccccc6)cccc45)c23)cc1 ; c1ccc(-c2cccc3c2oc2cccc(N(c4cccc5c4oc4ccccc45)c4cccc5c4sc4c(-c6ccccc6)cccc45)c23)cc1]

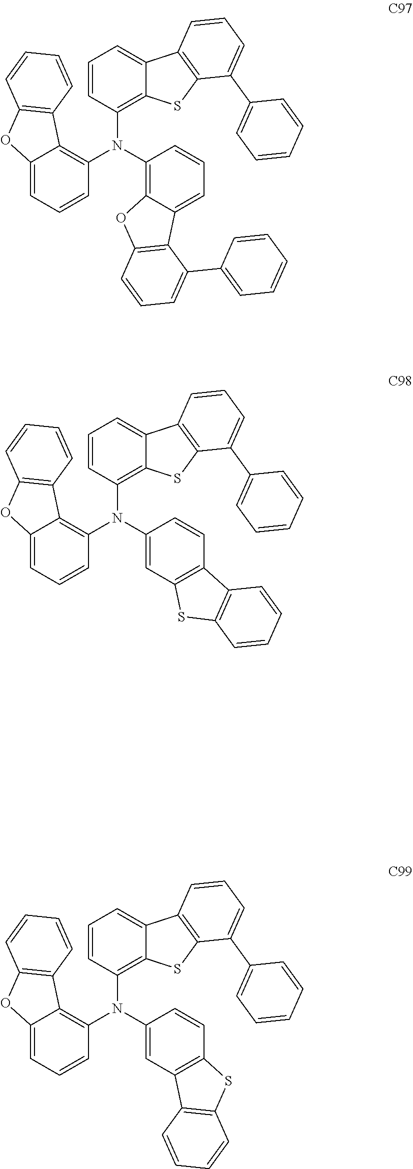 [SMILES: c1ccc(-c2cccc3c2sc2c(N(c4cccc5c4oc4cccc(-c6ccccc6)c45)c4cccc5oc6ccccc6c45)cccc23)cc1 ; c1ccc(-c2cccc3c2sc2c(N(c4ccc5c(c4)sc4ccccc45)c4cccc5oc6ccccc6c45)cccc23)cc1 ; c1ccc(-c2cccc3c2sc2c(N(c4ccc5sc6ccccc6c5c4)c4cccc5oc6ccccc6c45)cccc23)cc1]

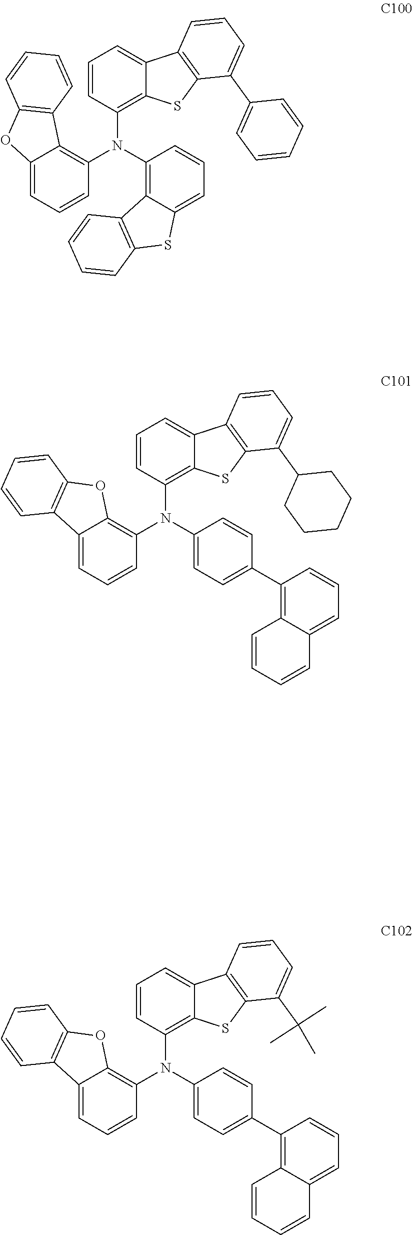 [SMILES: c1ccc(-c2cccc3c2sc2c(N(c4cccc5oc6ccccc6c45)c4cccc5sc6ccccc6c45)cccc23)cc1 ; c1ccc2c(-c3ccc(N(c4cccc5c4oc4ccccc45)c4cccc5c4sc4c(C6CCCCC6)cccc45)cc3)cccc2c1 ; CC(C)(C)c1cccc2c1sc1c(N(c3ccc(-c4cccc5ccccc45)cc3)c3cccc4c3oc3ccccc34)cccc12]

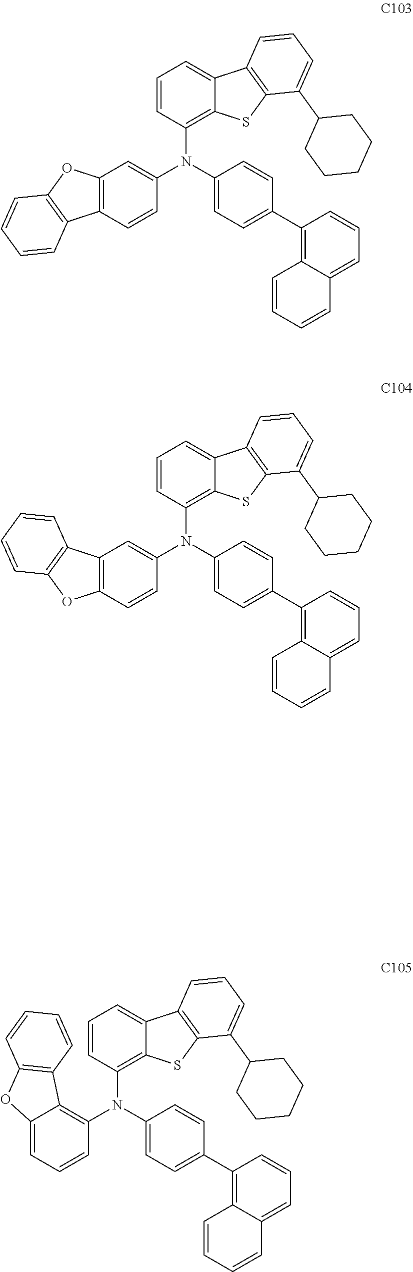 [SMILES: c1ccc2c(-c3ccc(N(c4ccc5c(c4)oc4ccccc45)c4cccc5c4sc4c(C6CCCCC6)cccc45)cc3)cccc2c1 ; c1ccc2c(-c3ccc(N(c4ccc5oc6ccccc6c5c4)c4cccc5c4sc4c(C6CCCCC6)cccc45)cc3)cccc2c1 ; c1ccc2c(-c3ccc(N(c4cccc5c4sc4c(C6CCCCC6)cccc45)c4cccc5oc6ccccc6c45)cc3)cccc2c1]

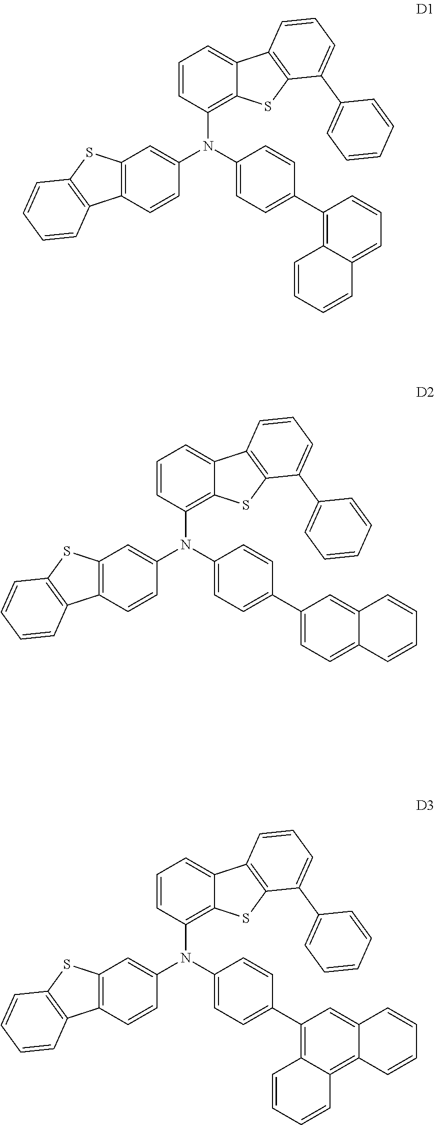 [SMILES: c1ccc(-c2cccc3c2sc2c(N(c4ccc(-c5cccc6ccccc56)cc4)c4ccc5c(c4)sc4ccccc45)cccc23)cc1 ; c1ccc(-c2cccc3c2sc2c(N(c4ccc(-c5ccc6ccccc6c5)cc4)c4ccc5c(c4)sc4ccccc45)cccc23)cc1 ; c1ccc(-c2cccc3c2sc2c(N(c4ccc(-c5cc6ccccc6c6ccccc56)cc4)c4ccc5c(c4)sc4ccccc45)cccc23)cc1]

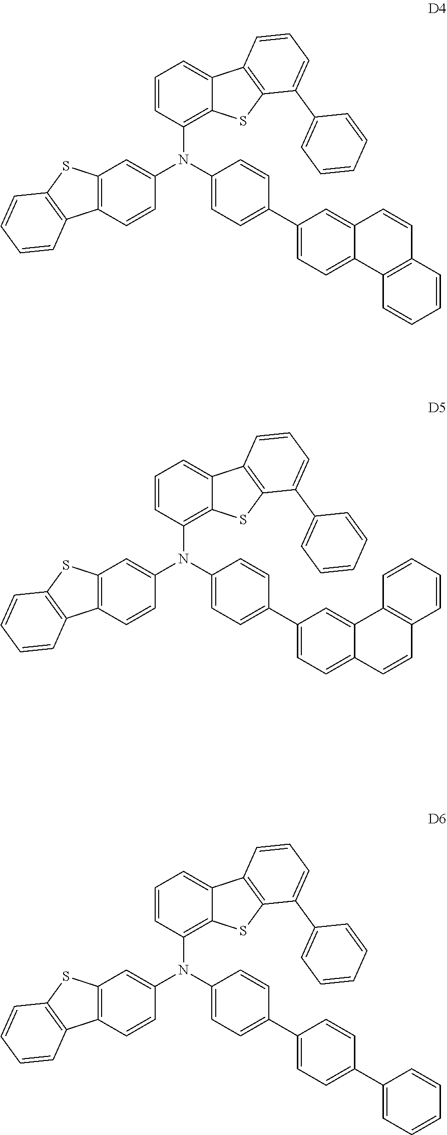 [SMILES: c1ccc(-c2cccc3c2sc2c(N(c4ccc(-c5ccc6c(ccc7ccccc76)c5)cc4)c4ccc5c(c4)sc4ccccc45)cccc23)cc1 ; c1ccc(-c2cccc3c2sc2c(N(c4ccc(-c5ccc6ccc7ccccc7c6c5)cc4)c4ccc5c(c4)sc4ccccc45)cccc23)cc1 ; c1ccc(-c2ccc(-c3ccc(N(c4ccc5c(c4)sc4ccccc45)c4cccc5c4sc4c(-c6ccccc6)cccc45)cc3)cc2)cc1]

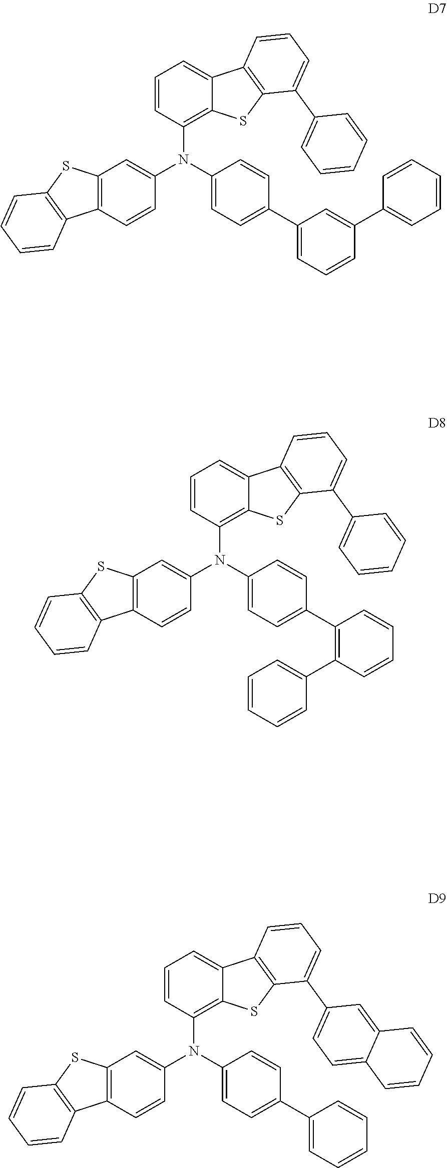 [SMILES: c1ccc(-c2cccc(-c3ccc(N(c4ccc5c(c4)sc4ccccc45)c4cccc5c4sc4c(-c6ccccc6)cccc45)cc3)c2)cc1 ; c1ccc(-c2ccccc2-c2ccc(N(c3ccc4c(c3)sc3ccccc34)c3cccc4c3sc3c(-c5ccccc5)cccc34)cc2)cc1 ; c1ccc(-c2ccc(N(c3ccc4c(c3)sc3ccccc34)c3cccc4c3sc3c(-c5ccc6ccccc6c5)cccc34)cc2)cc1]

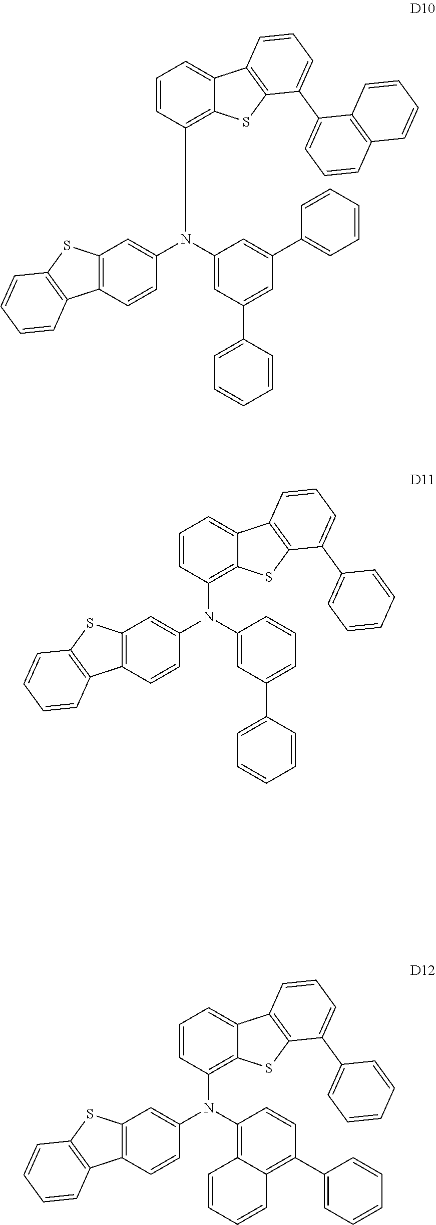 [SMILES: c1ccc(-c2cc(-c3ccccc3)cc(N(c3ccc4c(c3)sc3ccccc34)c3cccc4c3sc3c(-c5cccc6ccccc56)cccc34)c2)cc1 ; c1ccc(-c2cccc(N(c3ccc4c(c3)sc3ccccc34)c3cccc4c3sc3c(-c5ccccc5)cccc34)c2)cc1 ; c1ccc(-c2ccc(N(c3ccc4c(c3)sc3ccccc34)c3cccc4c3sc3c(-c5ccccc5)cccc34)c3ccccc23)cc1]

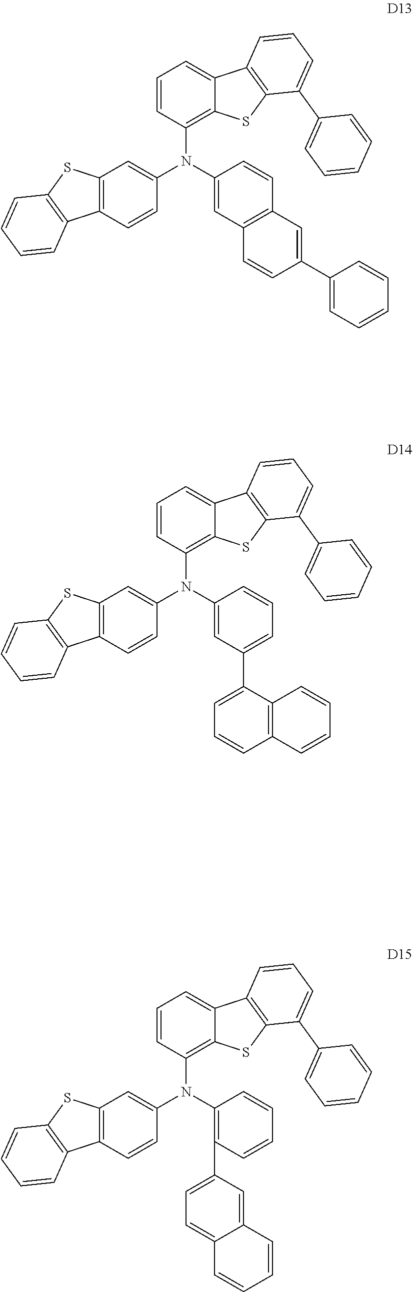 [SMILES: c1ccc(-c2ccc3cc(N(c4ccc5c(c4)sc4ccccc45)c4cccc5c4sc4c(-c6ccccc6)cccc45)ccc3c2)cc1 ; c1ccc(-c2cccc3c2sc2c(N(c4cccc(-c5cccc6ccccc56)c4)c4ccc5c(c4)sc4ccccc45)cccc23)cc1 ; c1ccc(-c2cccc3c2sc2c(N(c4ccc5c(c4)sc4ccccc45)c4ccccc4-c4ccc5ccccc5c4)cccc23)cc1]

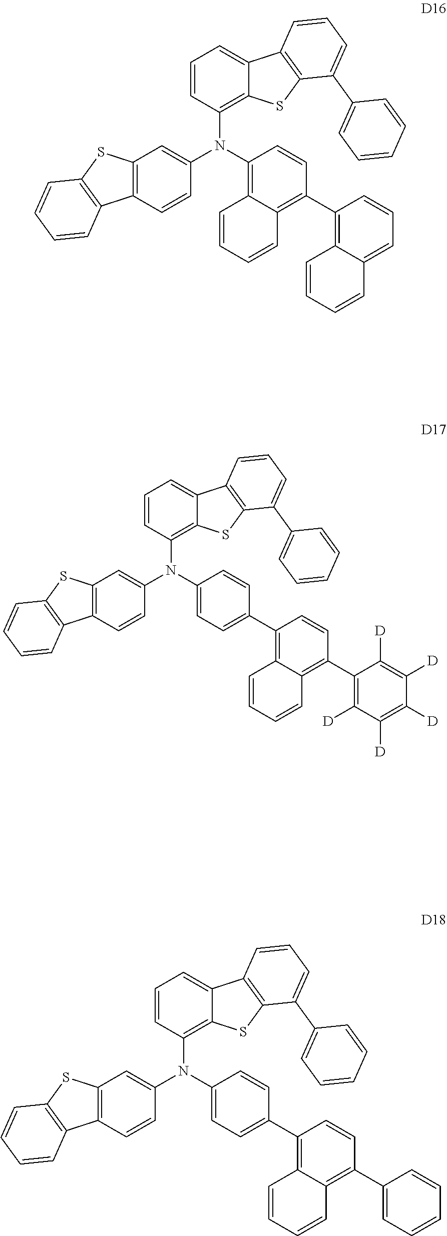 [SMILES: c1ccc(-c2cccc3c2sc2c(N(c4ccc5c(c4)sc4ccccc45)c4ccc(-c5cccc6ccccc56)c5ccccc45)cccc23)cc1 ; [2H]c1c([2H])c([2H])c(-c2ccc(-c3ccc(N(c4ccc5c(c4)sc4ccccc45)c4cccc5c4sc4c(-c6ccccc6)cccc45)cc3)c3ccccc23)c([2H])c1[2H] ; c1ccc(-c2ccc(-c3ccc(N(c4ccc5c(c4)sc4ccccc45)c4cccc5c4sc4c(-c6ccccc6)cccc45)cc3)c3ccccc23)cc1]

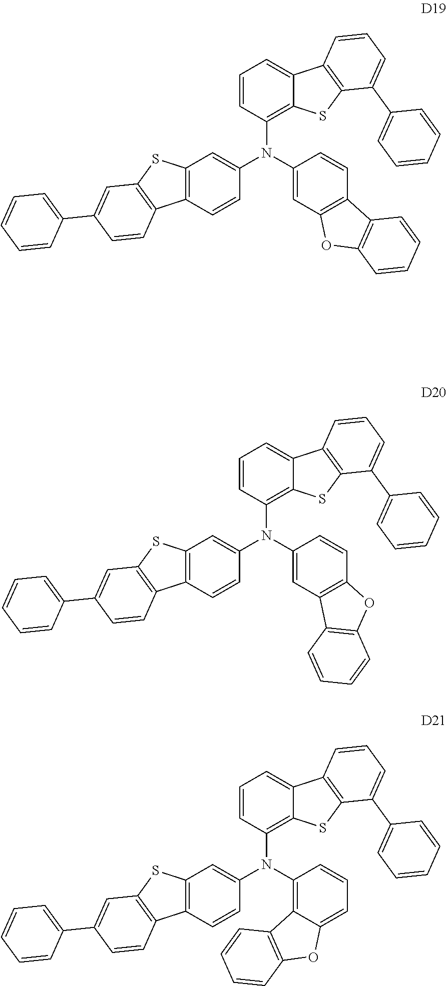 [SMILES: c1ccc(-c2ccc3c(c2)sc2cc(N(c4ccc5c(c4)oc4ccccc45)c4cccc5c4sc4c(-c6ccccc6)cccc45)ccc23)cc1 ; c1ccc(-c2ccc3c(c2)sc2cc(N(c4ccc5oc6ccccc6c5c4)c4cccc5c4sc4c(-c6ccccc6)cccc45)ccc23)cc1 ; c1ccc(-c2ccc3c(c2)sc2cc(N(c4cccc5c4sc4c(-c6ccccc6)cccc45)c4cccc5oc6ccccc6c45)ccc23)cc1]

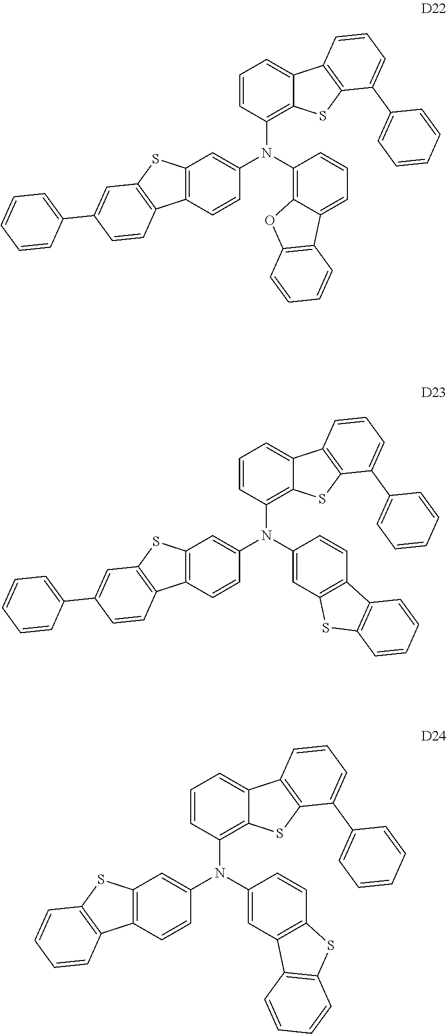 [SMILES: c1ccc(-c2ccc3c(c2)sc2cc(N(c4cccc5c4oc4ccccc45)c4cccc5c4sc4c(-c6ccccc6)cccc45)ccc23)cc1 ; c1ccc(-c2ccc3c(c2)sc2cc(N(c4ccc5c(c4)sc4ccccc45)c4cccc5c4sc4c(-c6ccccc6)cccc45)ccc23)cc1 ; c1ccc(-c2cccc3c2sc2c(N(c4ccc5c(c4)sc4ccccc45)c4ccc5sc6ccccc6c5c4)cccc23)cc1]

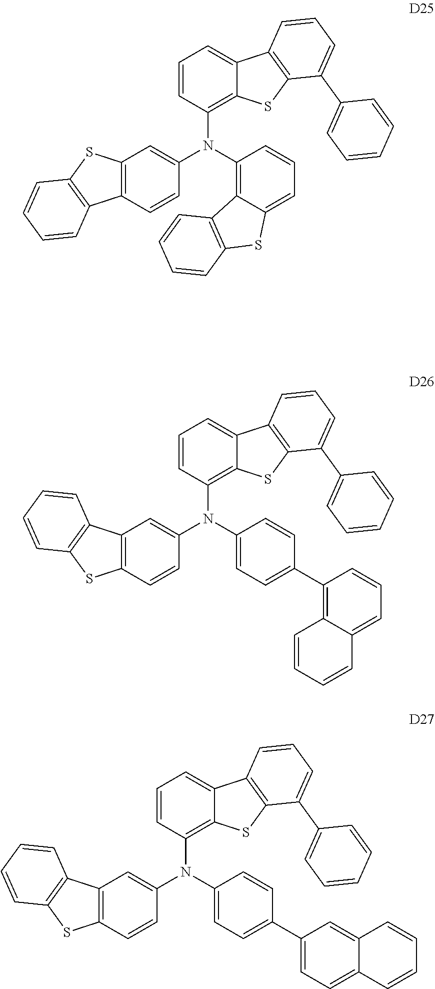 [SMILES: c1ccc(-c2cccc3c2sc2c(N(c4ccc5c(c4)sc4ccccc45)c4cccc5sc6ccccc6c45)cccc23)cc1 ; c1ccc(-c2cccc3c2sc2c(N(c4ccc(-c5cccc6ccccc56)cc4)c4ccc5sc6ccccc6c5c4)cccc23)cc1 ; c1ccc(-c2cccc3c2sc2c(N(c4ccc(-c5ccc6ccccc6c5)cc4)c4ccc5sc6ccccc6c5c4)cccc23)cc1]

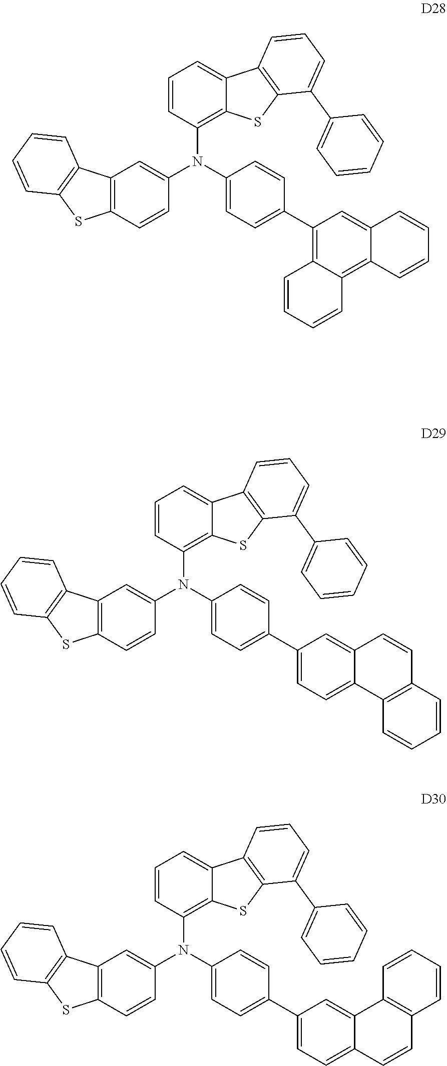 [SMILES: c1ccc(-c2cccc3c2sc2c(N(c4ccc(-c5cc6ccccc6c6ccccc56)cc4)c4ccc5sc6ccccc6c5c4)cccc23)cc1 ; c1ccc(-c2cccc3c2sc2c(N(c4ccc(-c5ccc6c(ccc7ccccc76)c5)cc4)c4ccc5sc6ccccc6c5c4)cccc23)cc1 ; c1ccc(-c2cccc3c2sc2c(N(c4ccc(-c5ccc6ccc7ccccc7c6c5)cc4)c4ccc5sc6ccccc6c5c4)cccc23)cc1]

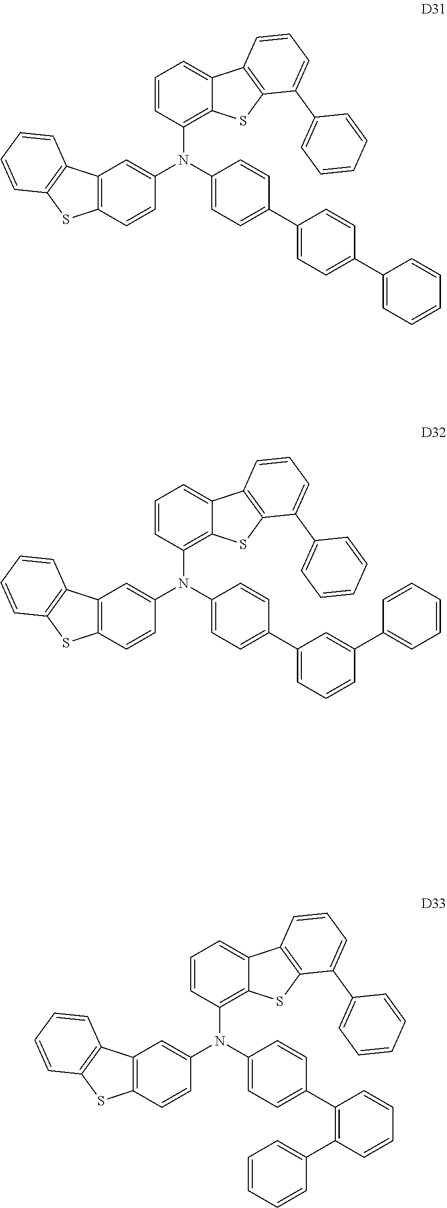 [SMILES: c1ccc(-c2ccc(-c3ccc(N(c4ccc5sc6ccccc6c5c4)c4cccc5c4sc4c(-c6ccccc6)cccc45)cc3)cc2)cc1 ; c1ccc(-c2cccc(-c3ccc(N(c4ccc5sc6ccccc6c5c4)c4cccc5c4sc4c(-c6ccccc6)cccc45)cc3)c2)cc1 ; c1ccc(-c2ccccc2-c2ccc(N(c3ccc4sc5ccccc5c4c3)c3cccc4c3sc3c(-c5ccccc5)cccc34)cc2)cc1]

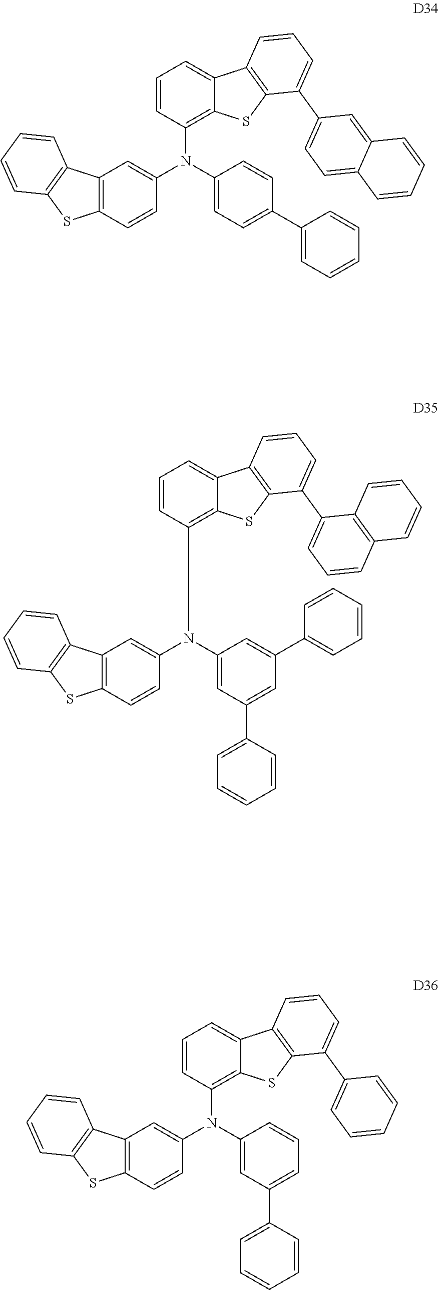 [SMILES: c1ccc(-c2ccc(N(c3ccc4sc5ccccc5c4c3)c3cccc4c3sc3c(-c5ccc6ccccc6c5)cccc34)cc2)cc1 ; c1ccc(-c2cc(-c3ccccc3)cc(N(c3ccc4sc5ccccc5c4c3)c3cccc4c3sc3c(-c5cccc6ccccc56)cccc34)c2)cc1 ; c1ccc(-c2cccc(N(c3ccc4sc5ccccc5c4c3)c3cccc4c3sc3c(-c5ccccc5)cccc34)c2)cc1]

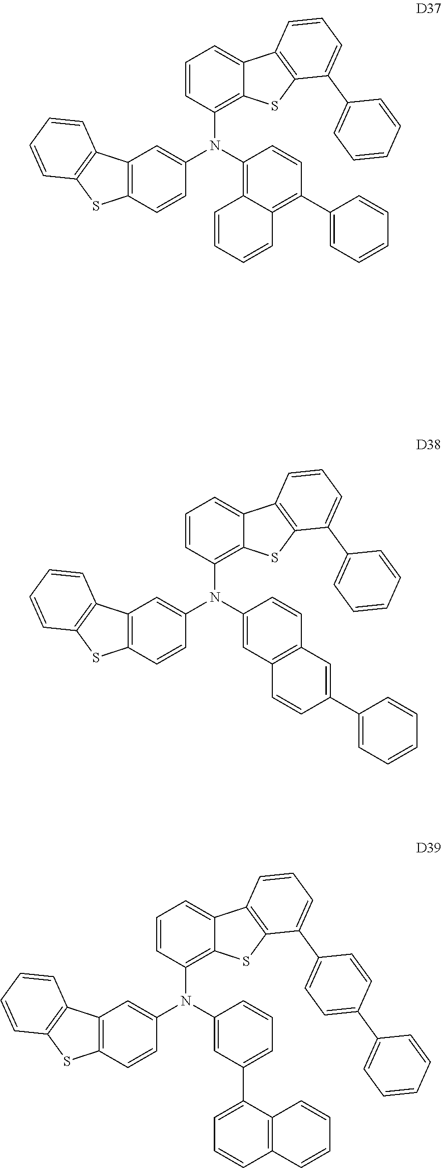 [SMILES: c1ccc(-c2ccc(N(c3ccc4sc5ccccc5c4c3)c3cccc4c3sc3c(-c5ccccc5)cccc34)c3ccccc23)cc1 ; c1ccc(-c2ccc3cc(N(c4ccc5sc6ccccc6c5c4)c4cccc5c4sc4c(-c6ccccc6)cccc45)ccc3c2)cc1 ; c1ccc(-c2ccc(-c3cccc4c3sc3c(N(c5cccc(-c6cccc7ccccc67)c5)c5ccc6sc7ccccc7c6c5)cccc34)cc2)cc1]

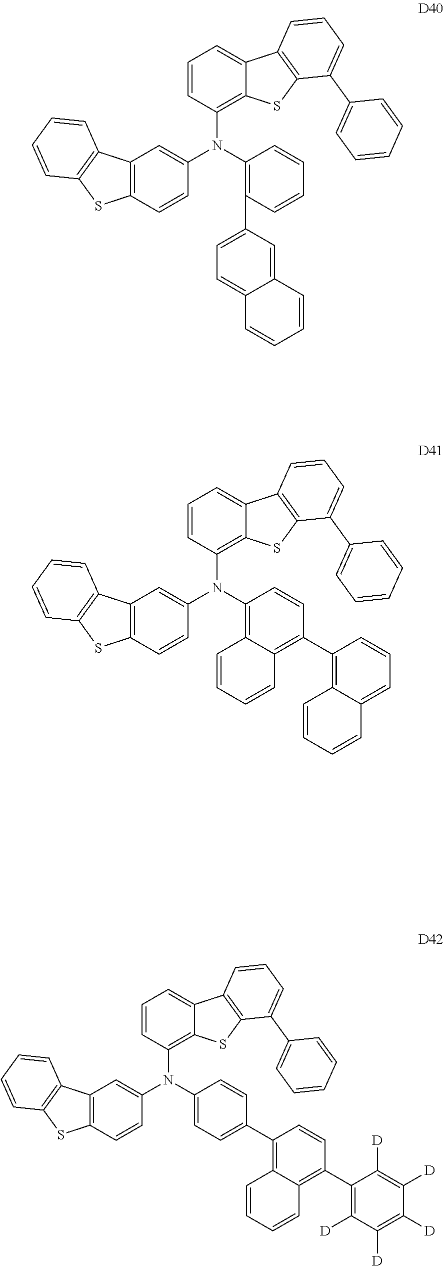 [SMILES: c1ccc(-c2cccc3c2sc2c(N(c4ccc5sc6ccccc6c5c4)c4ccccc4-c4ccc5ccccc5c4)cccc23)cc1 ; c1ccc(-c2cccc3c2sc2c(N(c4ccc5sc6ccccc6c5c4)c4ccc(-c5cccc6ccccc56)c5ccccc45)cccc23)cc1 ; [2H]c1c([2H])c([2H])c(-c2ccc(-c3ccc(N(c4ccc5sc6ccccc6c5c4)c4cccc5c4sc4c(-c6ccccc6)cccc45)cc3)c3ccccc23)c([2H])c1[2H]]

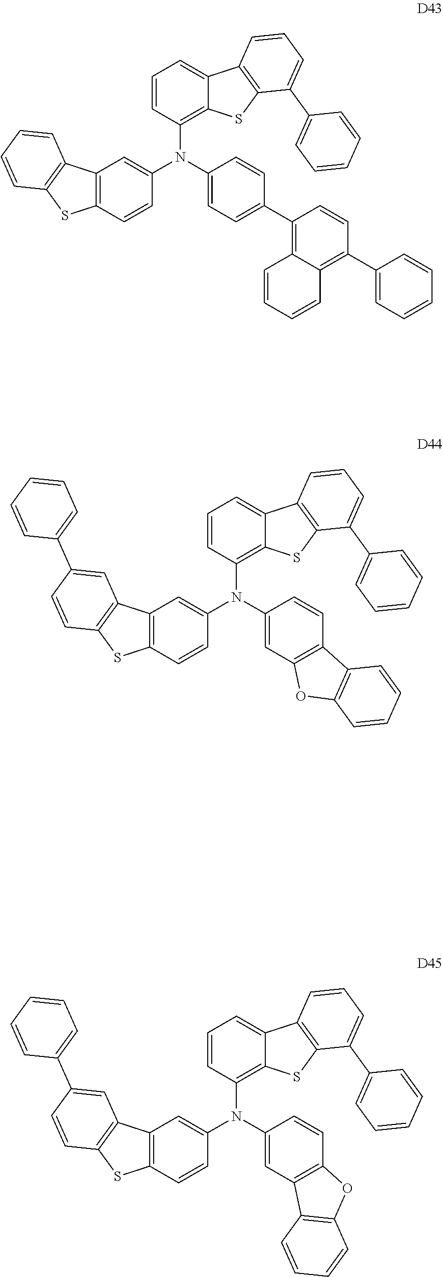 [SMILES: c1ccc(-c2ccc(-c3ccc(N(c4ccc5sc6ccccc6c5c4)c4cccc5c4sc4c(-c6ccccc6)cccc45)cc3)c3ccccc23)cc1 ; c1ccc(-c2ccc3sc4ccc(N(c5ccc6c(c5)oc5ccccc56)c5cccc6c5sc5c(-c7ccccc7)cccc56)cc4c3c2)cc1 ; c1ccc(-c2ccc3sc4ccc(N(c5ccc6oc7ccccc7c6c5)c5cccc6c5sc5c(-c7ccccc7)cccc56)cc4c3c2)cc1]

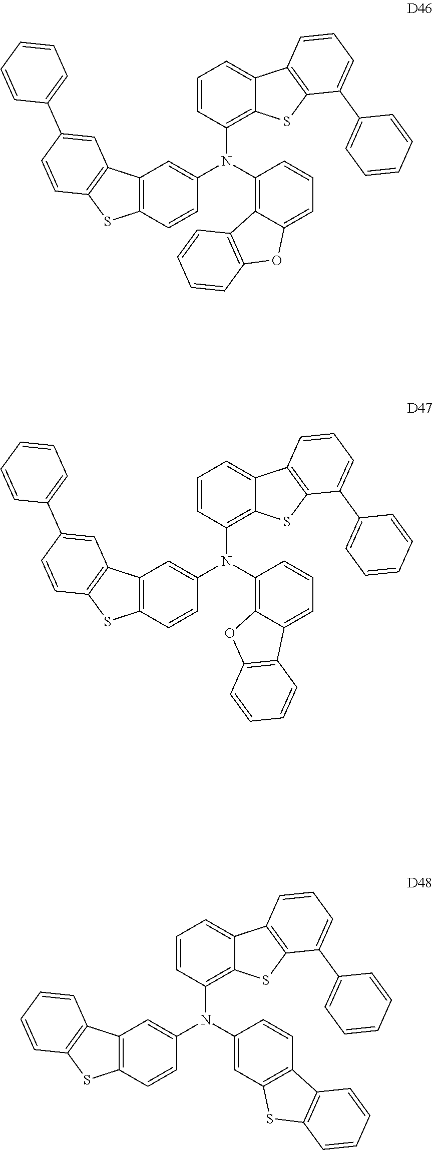 [SMILES: c1ccc(-c2ccc3sc4ccc(N(c5cccc6c5sc5c(-c7ccccc7)cccc56)c5cccc6oc7ccccc7c56)cc4c3c2)cc1 ; c1ccc(-c2ccc3sc4ccc(N(c5cccc6c5oc5ccccc56)c5cccc6c5sc5c(-c7ccccc7)cccc56)cc4c3c2)cc1 ; c1ccc(-c2cccc3c2sc2c(N(c4ccc5c(c4)sc4ccccc45)c4ccc5sc6ccccc6c5c4)cccc23)cc1]

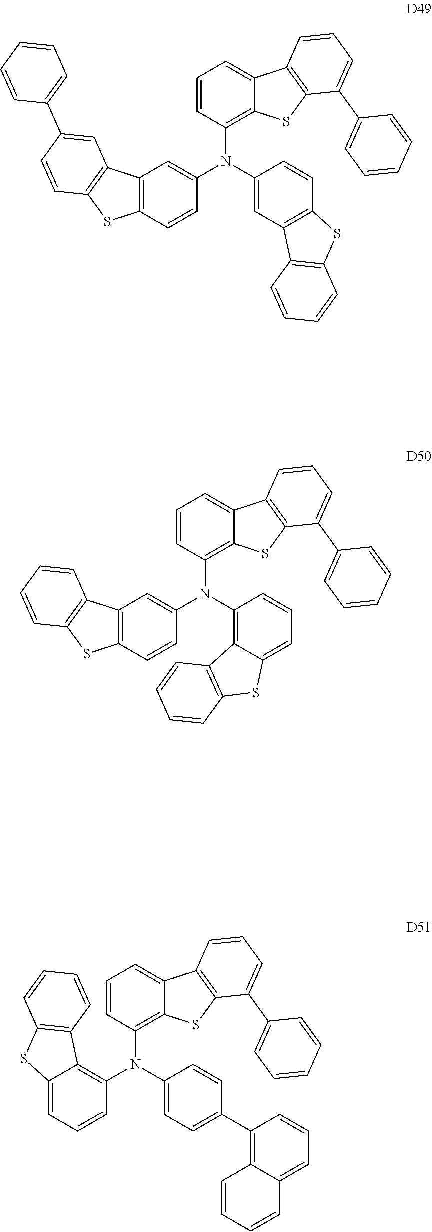 [SMILES: c1ccc(-c2ccc3sc4ccc(N(c5ccc6sc7ccccc7c6c5)c5cccc6c5sc5c(-c7ccccc7)cccc56)cc4c3c2)cc1 ; c1ccc(-c2cccc3c2sc2c(N(c4ccc5sc6ccccc6c5c4)c4cccc5sc6ccccc6c45)cccc23)cc1 ; c1ccc(-c2cccc3c2sc2c(N(c4ccc(-c5cccc6ccccc56)cc4)c4cccc5sc6ccccc6c45)cccc23)cc1]

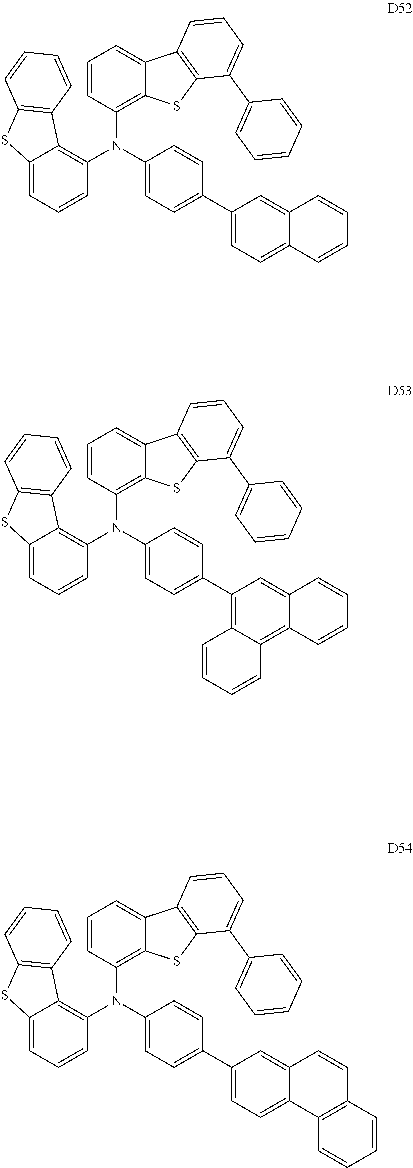 [SMILES: c1ccc(-c2cccc3c2sc2c(N(c4ccc(-c5ccc6ccccc6c5)cc4)c4cccc5sc6ccccc6c45)cccc23)cc1 ; c1ccc(-c2cccc3c2sc2c(N(c4ccc(-c5cc6ccccc6c6ccccc56)cc4)c4cccc5sc6ccccc6c45)cccc23)cc1 ; c1ccc(-c2cccc3c2sc2c(N(c4ccc(-c5ccc6c(ccc7ccccc76)c5)cc4)c4cccc5sc6ccccc6c45)cccc23)cc1]

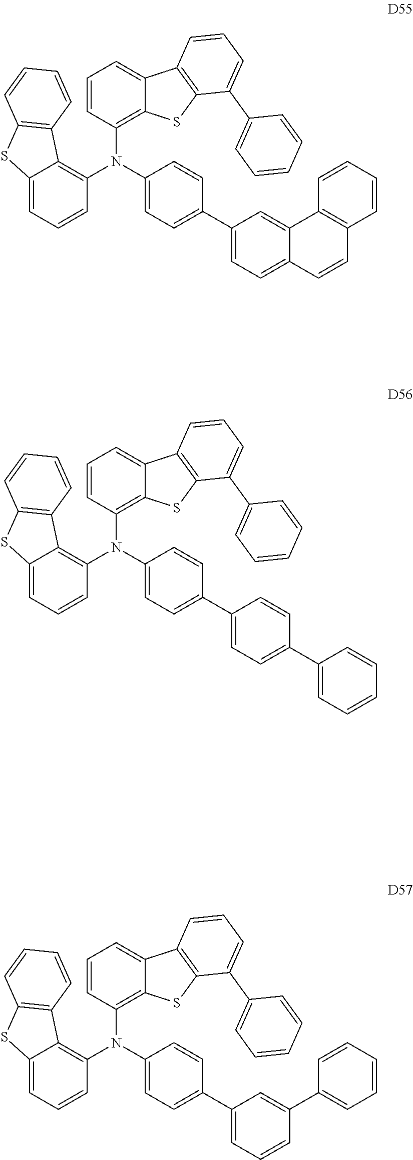 [SMILES: c1ccc(-c2cccc3c2sc2c(N(c4ccc(-c5ccc6ccc7ccccc7c6c5)cc4)c4cccc5sc6ccccc6c45)cccc23)cc1 ; c1ccc(-c2ccc(-c3ccc(N(c4cccc5c4sc4c(-c6ccccc6)cccc45)c4cccc5sc6ccccc6c45)cc3)cc2)cc1 ; c1ccc(-c2cccc(-c3ccc(N(c4cccc5c4sc4c(-c6ccccc6)cccc45)c4cccc5sc6ccccc6c45)cc3)c2)cc1]

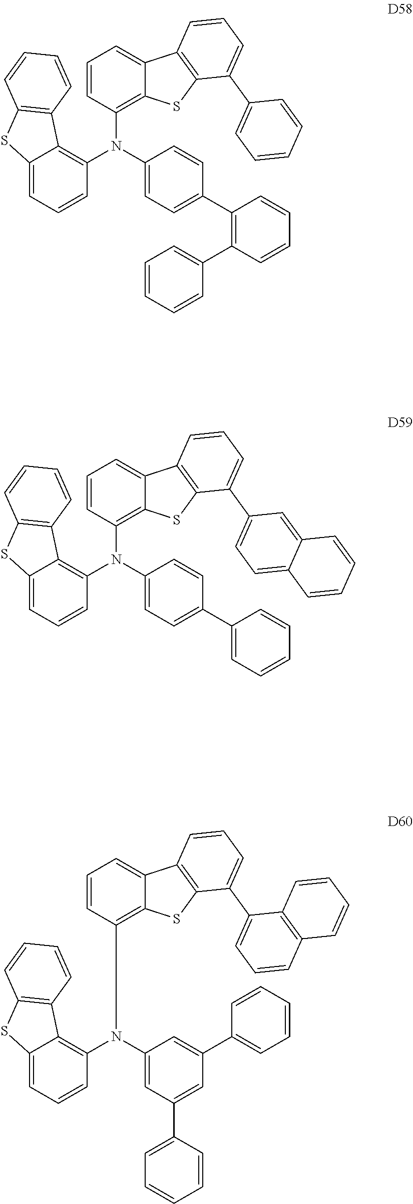 [SMILES: c1ccc(-c2ccccc2-c2ccc(N(c3cccc4c3sc3c(-c5ccccc5)cccc34)c3cccc4sc5ccccc5c34)cc2)cc1 ; c1ccc(-c2ccc(N(c3cccc4c3sc3c(-c5ccc6ccccc6c5)cccc34)c3cccc4sc5ccccc5c34)cc2)cc1 ; c1ccc(-c2cc(-c3ccccc3)cc(N(c3cccc4c3sc3c(-c5cccc6ccccc56)cccc34)c3cccc4sc5ccccc5c34)c2)cc1]

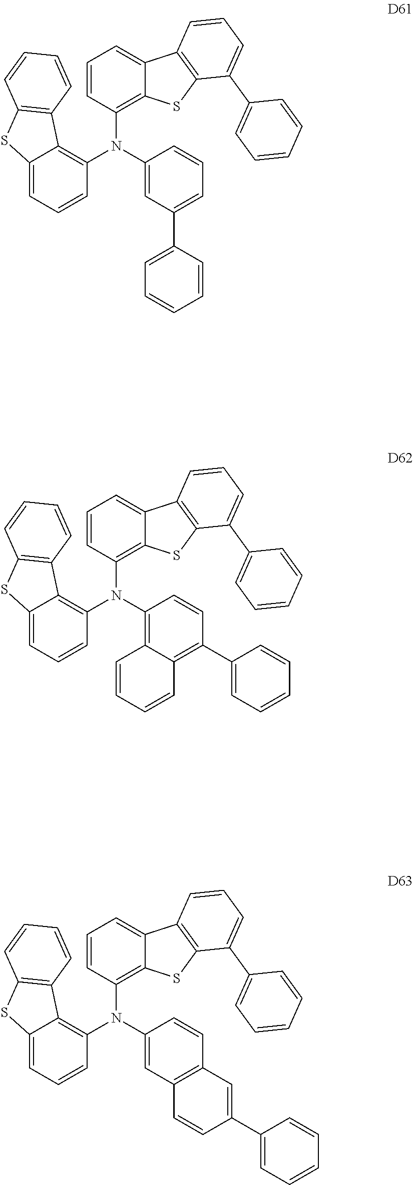 [SMILES: c1ccc(-c2cccc(N(c3cccc4c3sc3c(-c5ccccc5)cccc34)c3cccc4sc5ccccc5c34)c2)cc1 ; c1ccc(-c2ccc(N(c3cccc4c3sc3c(-c5ccccc5)cccc34)c3cccc4sc5ccccc5c34)c3ccccc23)cc1 ; c1ccc(-c2ccc3cc(N(c4cccc5c4sc4c(-c6ccccc6)cccc45)c4cccc5sc6ccccc6c45)ccc3c2)cc1]

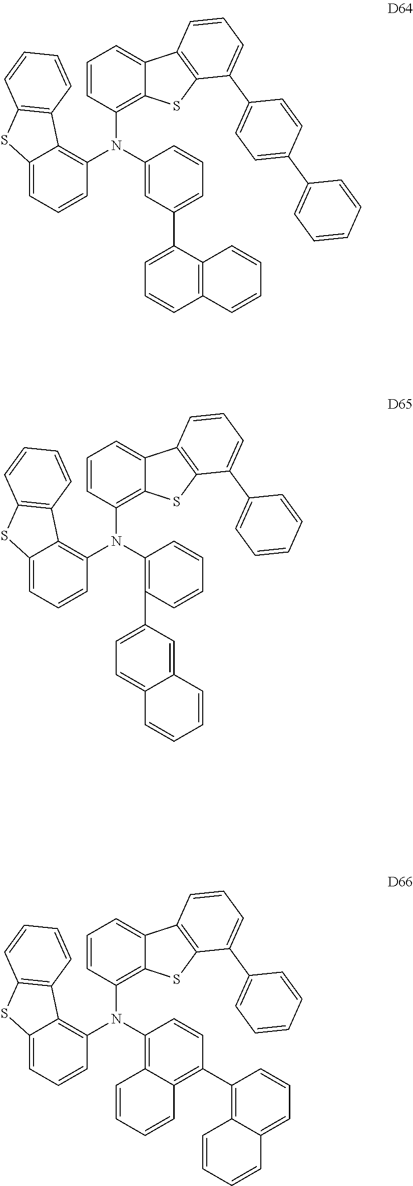 [SMILES: c1ccc(-c2ccc(-c3cccc4c3sc3c(N(c5cccc(-c6cccc7ccccc67)c5)c5cccc6sc7ccccc7c56)cccc34)cc2)cc1 ; c1ccc(-c2cccc3c2sc2c(N(c4ccccc4-c4ccc5ccccc5c4)c4cccc5sc6ccccc6c45)cccc23)cc1 ; c1ccc(-c2cccc3c2sc2c(N(c4ccc(-c5cccc6ccccc56)c5ccccc45)c4cccc5sc6ccccc6c45)cccc23)cc1]

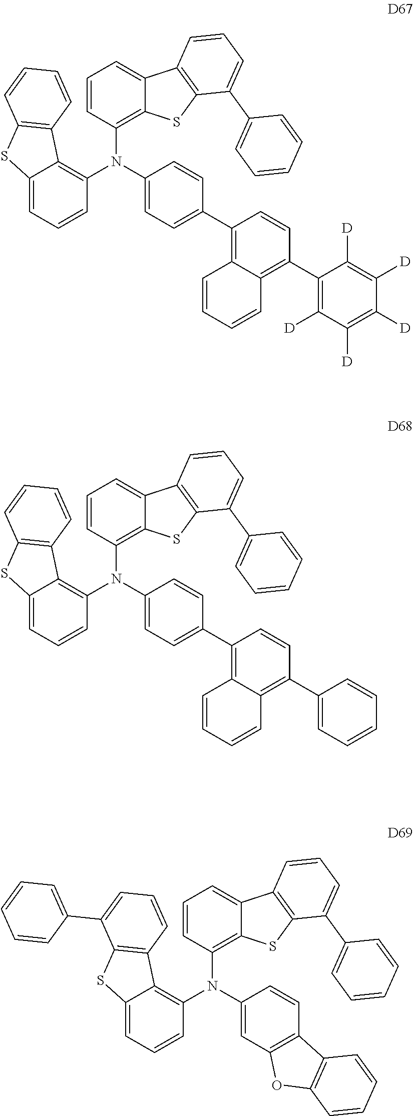 [SMILES: [2H]c1c([2H])c([2H])c(-c2ccc(-c3ccc(N(c4cccc5c4sc4c(-c6ccccc6)cccc45)c4cccc5sc6ccccc6c45)cc3)c3ccccc23)c([2H])c1[2H] ; c1ccc(-c2ccc(-c3ccc(N(c4cccc5c4sc4c(-c6ccccc6)cccc45)c4cccc5sc6ccccc6c45)cc3)c3ccccc23)cc1 ; c1ccc(-c2cccc3c2sc2c(N(c4ccc5c(c4)oc4ccccc45)c4cccc5sc6c(-c7ccccc7)cccc6c45)cccc23)cc1]

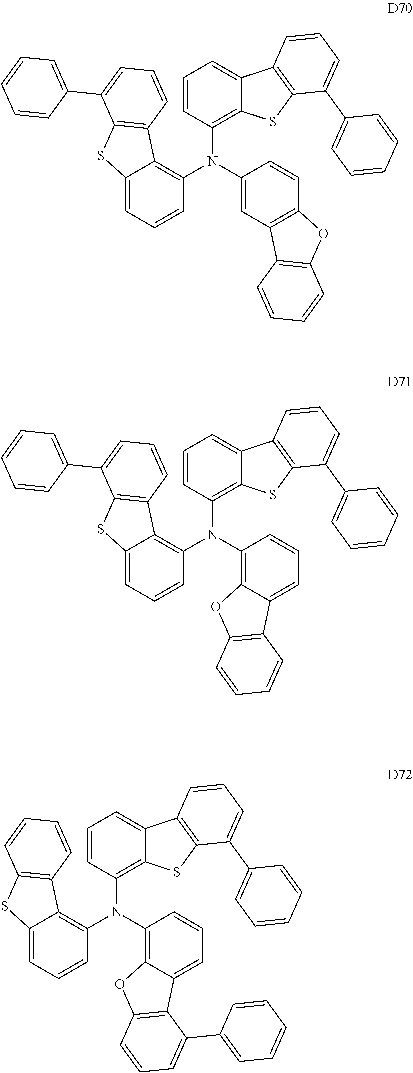 [SMILES: c1ccc(-c2cccc3c2sc2c(N(c4ccc5oc6ccccc6c5c4)c4cccc5sc6c(-c7ccccc7)cccc6c45)cccc23)cc1 ; c1ccc(-c2cccc3c2sc2c(N(c4cccc5c4oc4ccccc45)c4cccc5sc6c(-c7ccccc7)cccc6c45)cccc23)cc1 ; c1ccc(-c2cccc3c2sc2c(N(c4cccc5c4oc4cccc(-c6ccccc6)c45)c4cccc5sc6ccccc6c45)cccc23)cc1]

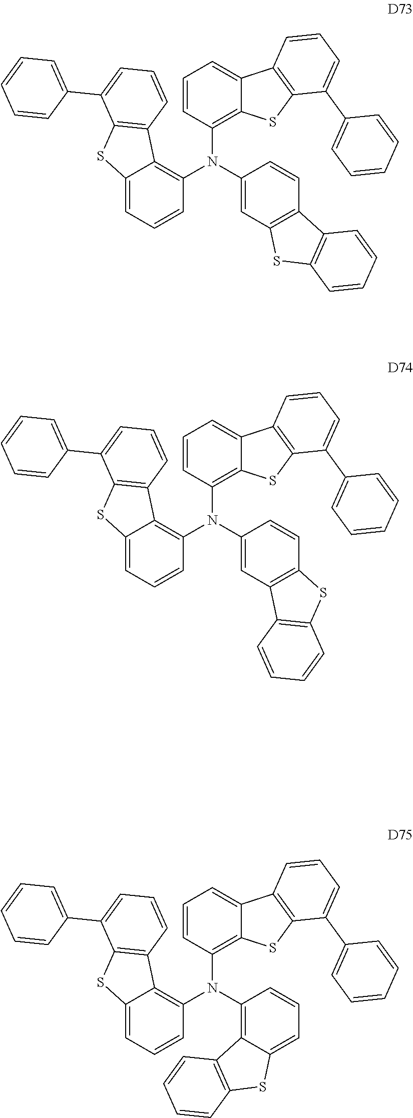 [SMILES: c1ccc(-c2cccc3c2sc2c(N(c4ccc5c(c4)sc4ccccc45)c4cccc5sc6c(-c7ccccc7)cccc6c45)cccc23)cc1 ; c1ccc(-c2cccc3c2sc2c(N(c4ccc5sc6ccccc6c5c4)c4cccc5sc6c(-c7ccccc7)cccc6c45)cccc23)cc1 ; c1ccc(-c2cccc3c2sc2c(N(c4cccc5sc6ccccc6c45)c4cccc5sc6c(-c7ccccc7)cccc6c45)cccc23)cc1]

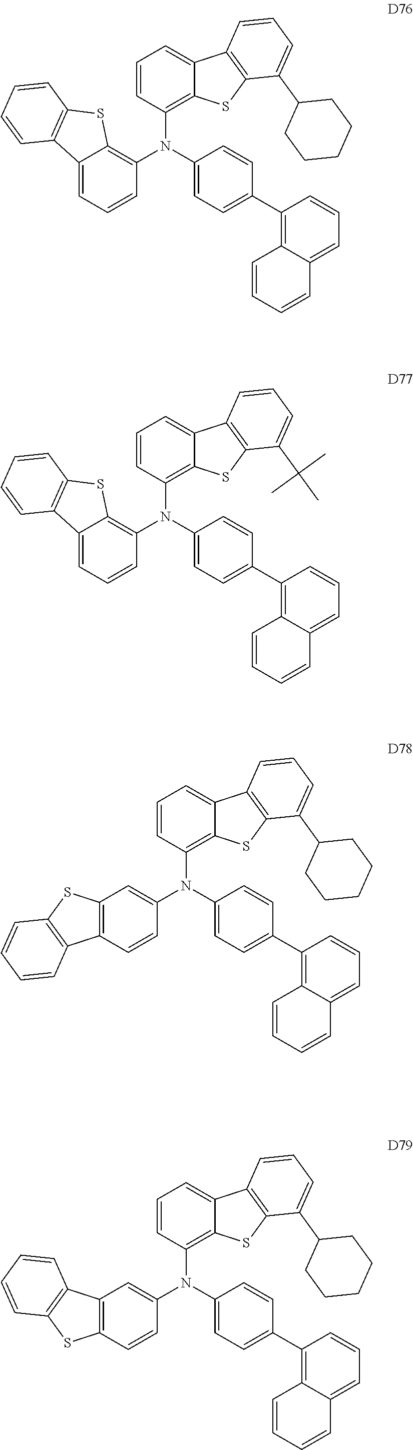 [SMILES: c1ccc2c(-c3ccc(N(c4cccc5c4sc4ccccc45)c4cccc5c4sc4c(C6CCCCC6)cccc45)cc3)cccc2c1 ; CC(C)(C)c1cccc2c1sc1c(N(c3ccc(-c4cccc5ccccc45)cc3)c3cccc4c3sc3ccccc34)cccc12 ; c1ccc2c(-c3ccc(N(c4ccc5c(c4)sc4ccccc45)c4cccc5c4sc4c(C6CCCCC6)cccc45)cc3)cccc2c1 ; c1ccc2c(-c3ccc(N(c4ccc5sc6ccccc6c5c4)c4cccc5c4sc4c(C6CCCCC6)cccc45)cc3)cccc2c1]

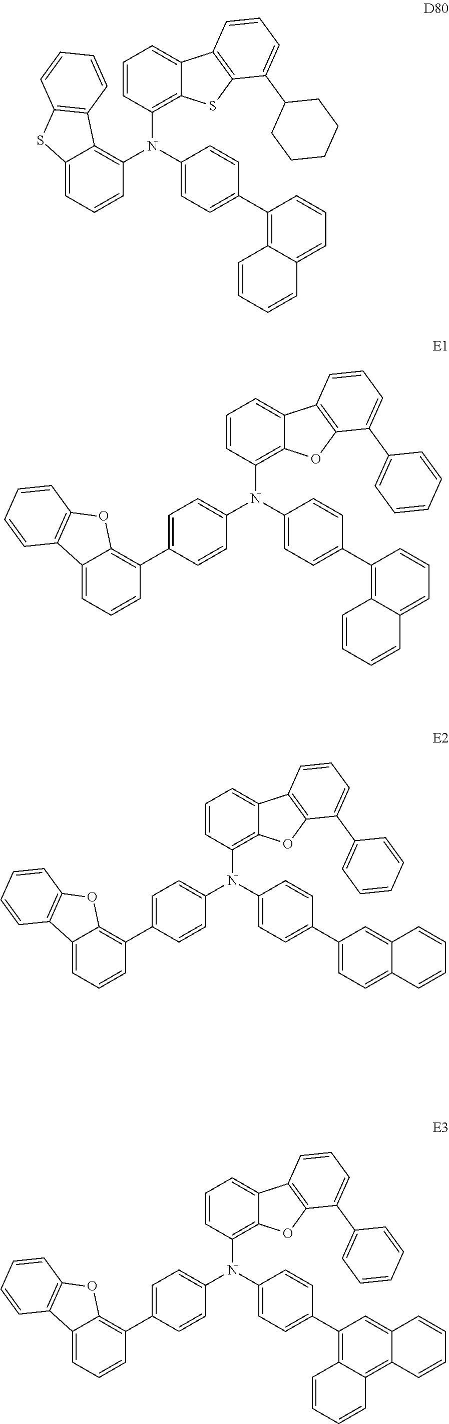 [SMILES: c1ccc2c(-c3ccc(N(c4cccc5c4sc4c(C6CCCCC6)cccc45)c4cccc5sc6ccccc6c45)cc3)cccc2c1 ; c1ccc(-c2cccc3c2oc2c(N(c4ccc(-c5cccc6ccccc56)cc4)c4ccc(-c5cccc6c5oc5ccccc56)cc4)cccc23)cc1 ; c1ccc(-c2cccc3c2oc2c(N(c4ccc(-c5ccc6ccccc6c5)cc4)c4ccc(-c5cccc6c5oc5ccccc56)cc4)cccc23)cc1 ; c1ccc(-c2cccc3c2oc2c(N(c4ccc(-c5cc6ccccc6c6ccccc56)cc4)c4ccc(-c5cccc6c5oc5ccccc56)cc4)cccc23)cc1]

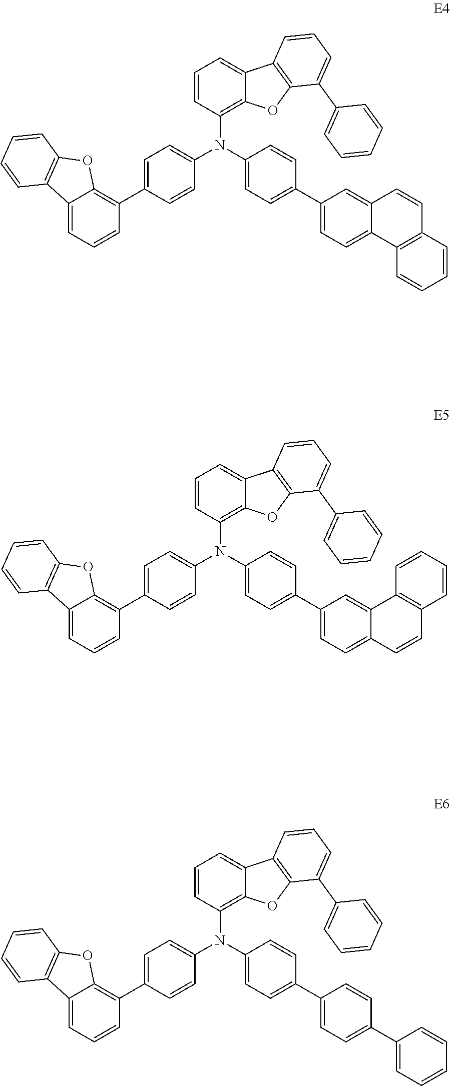 [SMILES: c1ccc(-c2cccc3c2oc2c(N(c4ccc(-c5ccc6c(ccc7ccccc76)c5)cc4)c4ccc(-c5cccc6c5oc5ccccc56)cc4)cccc23)cc1 ; c1ccc(-c2cccc3c2oc2c(N(c4ccc(-c5ccc6ccc7ccccc7c6c5)cc4)c4ccc(-c5cccc6c5oc5ccccc56)cc4)cccc23)cc1 ; c1ccc(-c2ccc(-c3ccc(N(c4ccc(-c5cccc6c5oc5ccccc56)cc4)c4cccc5c4oc4c(-c6ccccc6)cccc45)cc3)cc2)cc1]

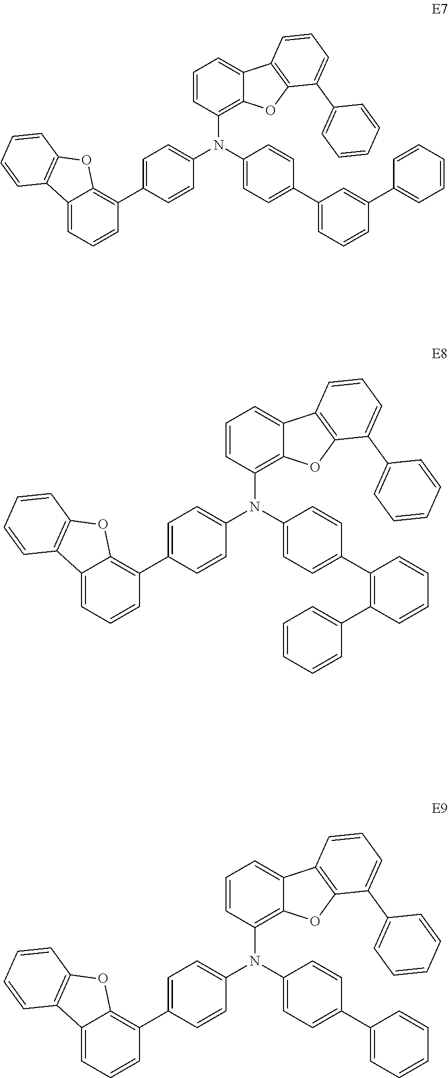 [SMILES: c1ccc(-c2cccc(-c3ccc(N(c4ccc(-c5cccc6c5oc5ccccc56)cc4)c4cccc5c4oc4c(-c6ccccc6)cccc45)cc3)c2)cc1 ; c1ccc(-c2ccccc2-c2ccc(N(c3ccc(-c4cccc5c4oc4ccccc45)cc3)c3cccc4c3oc3c(-c5ccccc5)cccc34)cc2)cc1 ; c1ccc(-c2ccc(N(c3ccc(-c4cccc5c4oc4ccccc45)cc3)c3cccc4c3oc3c(-c5ccccc5)cccc34)cc2)cc1]

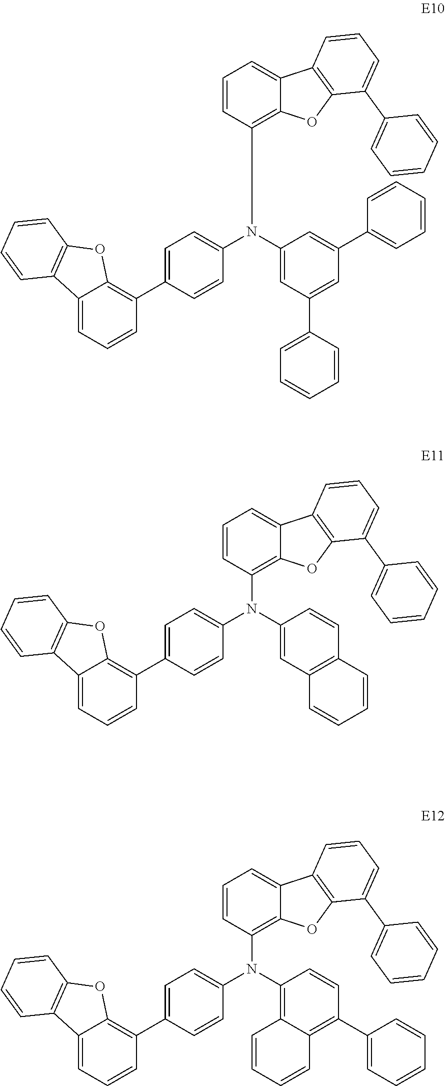 [SMILES: c1ccc(-c2cc(-c3ccccc3)cc(N(c3ccc(-c4cccc5c4oc4ccccc45)cc3)c3cccc4c3oc3c(-c5ccccc5)cccc34)c2)cc1 ; c1ccc(-c2cccc3c2oc2c(N(c4ccc(-c5cccc6c5oc5ccccc56)cc4)c4ccc5ccccc5c4)cccc23)cc1 ; c1ccc(-c2ccc(N(c3ccc(-c4cccc5c4oc4ccccc45)cc3)c3cccc4c3oc3c(-c5ccccc5)cccc34)c3ccccc23)cc1]

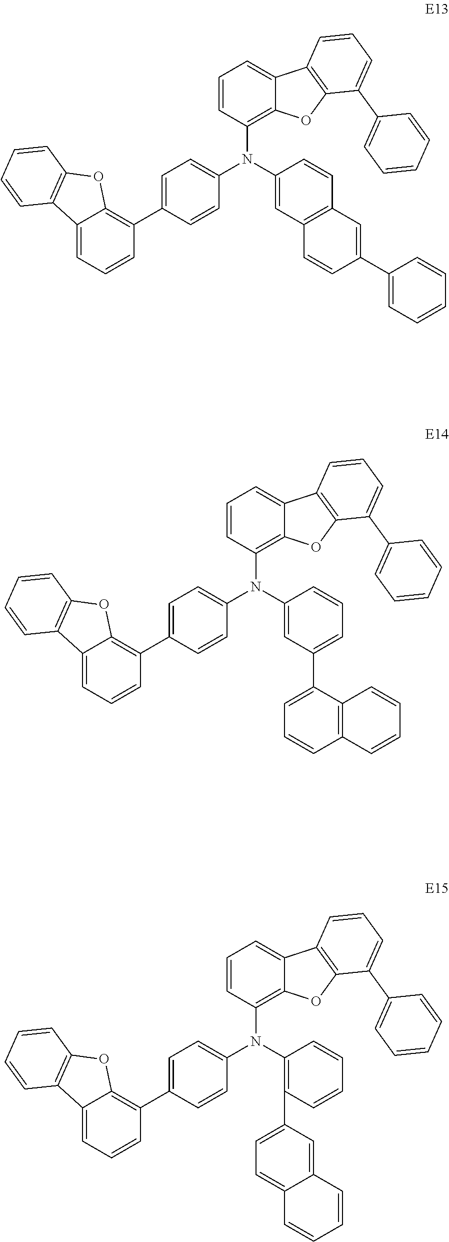 [SMILES: c1ccc(-c2ccc3cc(N(c4ccc(-c5cccc6c5oc5ccccc56)cc4)c4cccc5c4oc4c(-c6ccccc6)cccc45)ccc3c2)cc1 ; c1ccc(-c2cccc3c2oc2c(N(c4ccc(-c5cccc6c5oc5ccccc56)cc4)c4cccc(-c5cccc6ccccc56)c4)cccc23)cc1 ; c1ccc(-c2cccc3c2oc2c(N(c4ccc(-c5cccc6c5oc5ccccc56)cc4)c4ccccc4-c4ccc5ccccc5c4)cccc23)cc1]

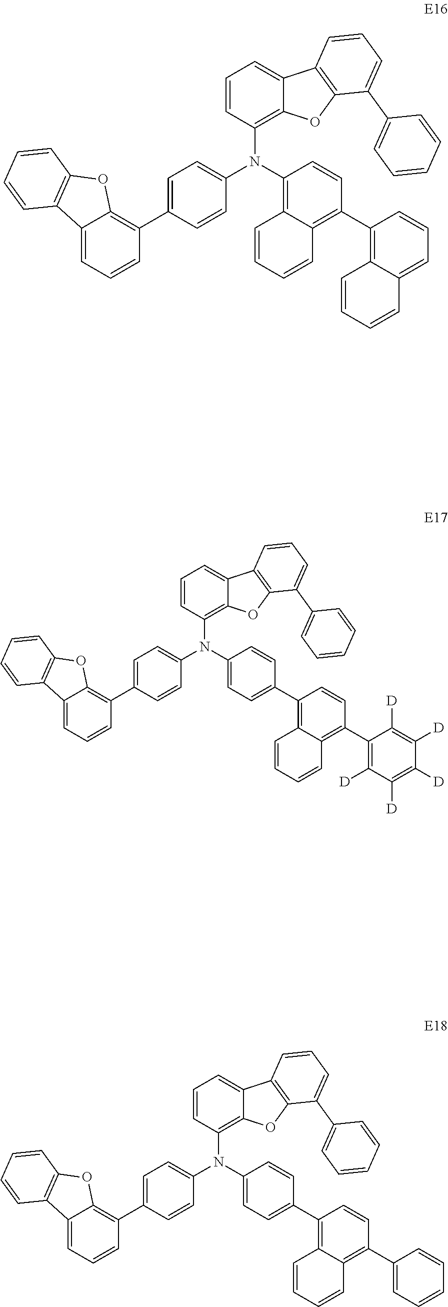 [SMILES: c1ccc(-c2cccc3c2oc2c(N(c4ccc(-c5cccc6c5oc5ccccc56)cc4)c4ccc(-c5cccc6ccccc56)c5ccccc45)cccc23)cc1 ; [2H]c1c([2H])c([2H])c(-c2ccc(-c3ccc(N(c4ccc(-c5cccc6c5oc5ccccc56)cc4)c4cccc5c4oc4c(-c6ccccc6)cccc45)cc3)c3ccccc23)c([2H])c1[2H] ; c1ccc(-c2ccc(-c3ccc(N(c4ccc(-c5cccc6c5oc5ccccc56)cc4)c4cccc5c4oc4c(-c6ccccc6)cccc45)cc3)c3ccccc23)cc1]

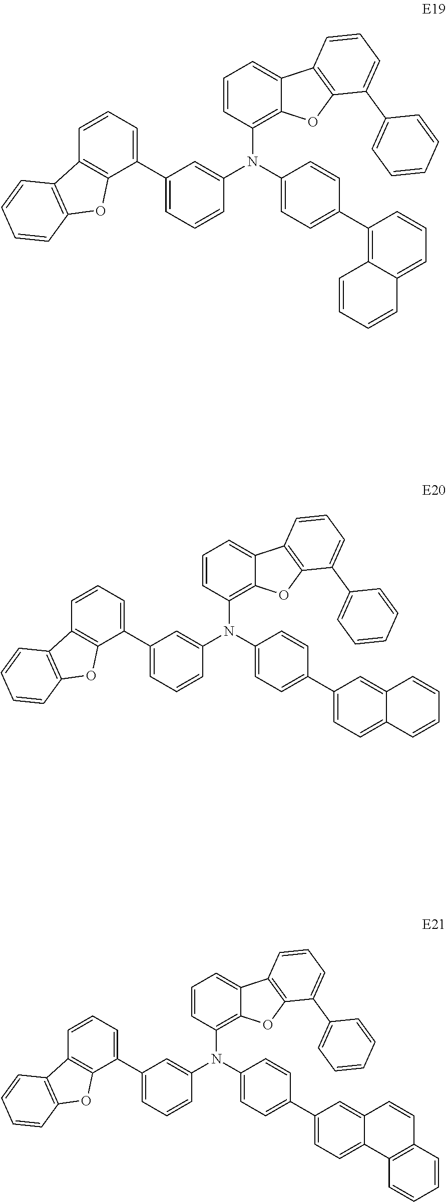 [SMILES: c1ccc(-c2cccc3c2oc2c(N(c4ccc(-c5cccc6ccccc56)cc4)c4cccc(-c5cccc6c5oc5ccccc56)c4)cccc23)cc1 ; c1ccc(-c2cccc3c2oc2c(N(c4ccc(-c5ccc6ccccc6c5)cc4)c4cccc(-c5cccc6c5oc5ccccc56)c4)cccc23)cc1 ; c1ccc(-c2cccc3c2oc2c(N(c4ccc(-c5ccc6c(ccc7ccccc76)c5)cc4)c4cccc(-c5cccc6c5oc5ccccc56)c4)cccc23)cc1]

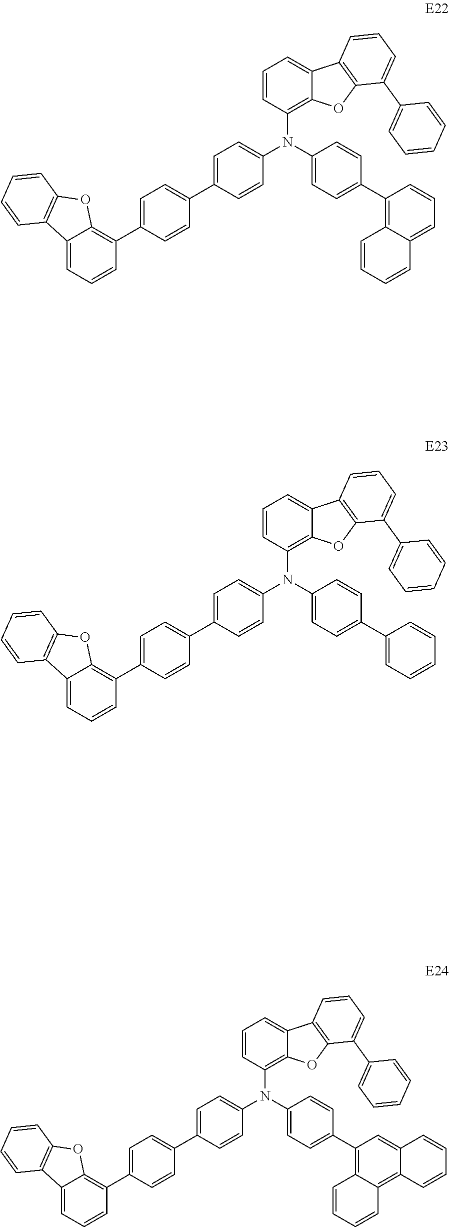 [SMILES: c1ccc(-c2cccc3c2oc2c(N(c4ccc(-c5ccc(-c6cccc7c6oc6ccccc67)cc5)cc4)c4ccc(-c5cccc6ccccc56)cc4)cccc23)cc1 ; c1ccc(-c2ccc(N(c3ccc(-c4ccc(-c5cccc6c5oc5ccccc56)cc4)cc3)c3cccc4c3oc3c(-c5ccccc5)cccc34)cc2)cc1 ; c1ccc(-c2cccc3c2oc2c(N(c4ccc(-c5ccc(-c6cccc7c6oc6ccccc67)cc5)cc4)c4ccc(-c5cc6ccccc6c6ccccc56)cc4)cccc23)cc1]

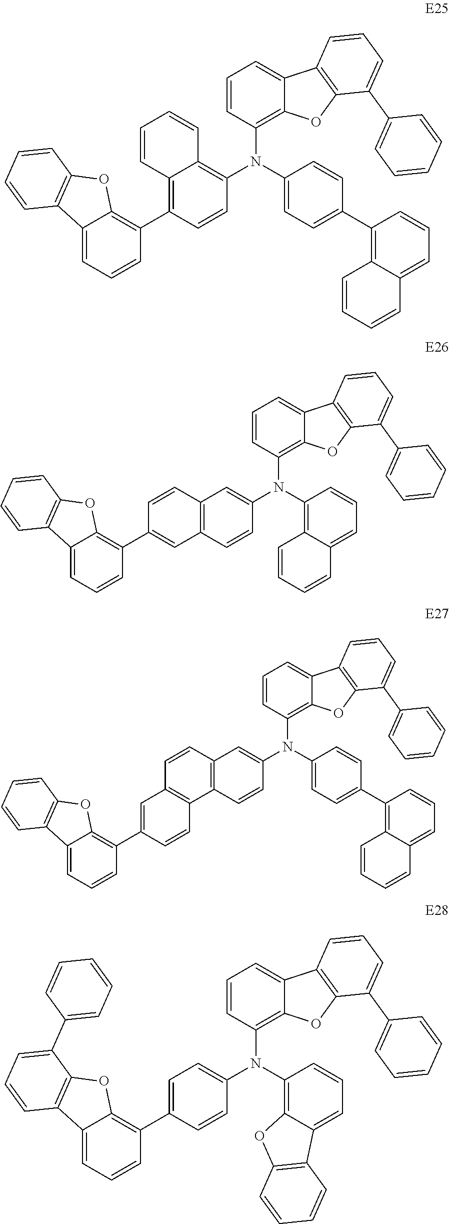

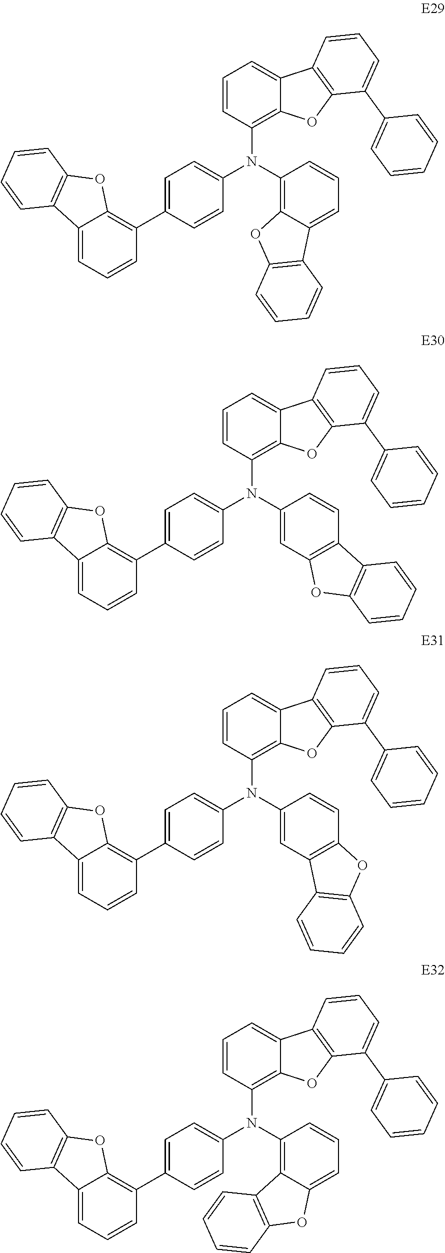

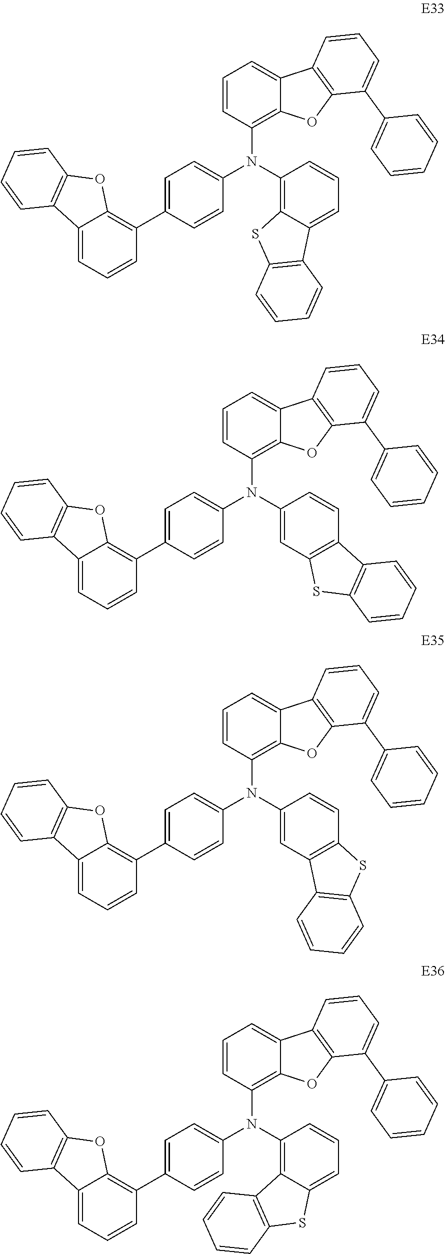

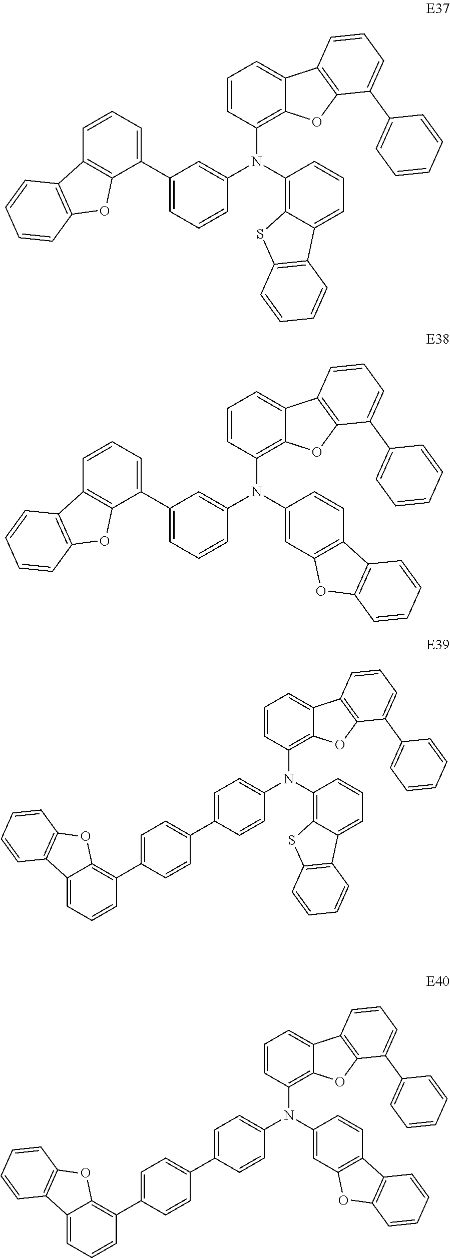 [SMILES: c1ccc(-c2cccc3c2oc2c(N(c4cccc(-c5cccc6c5oc5ccccc56)c4)c4cccc5c4sc4ccccc45)cccc23)cc1 ; c1ccc(-c2cccc3c2oc2c(N(c4cccc(-c5cccc6c5oc5ccccc56)c4)c4ccc5c(c4)oc4ccccc45)cccc23)cc1 ; c1ccc(-c2cccc3c2oc2c(N(c4ccc(-c5ccc(-c6cccc7c6oc6ccccc67)cc5)cc4)c4cccc5c4sc4ccccc45)cccc23)cc1 ; c1ccc(-c2cccc3c2oc2c(N(c4ccc(-c5ccc(-c6cccc7c6oc6ccccc67)cc5)cc4)c4ccc5c(c4)oc4ccccc45)cccc23)cc1]

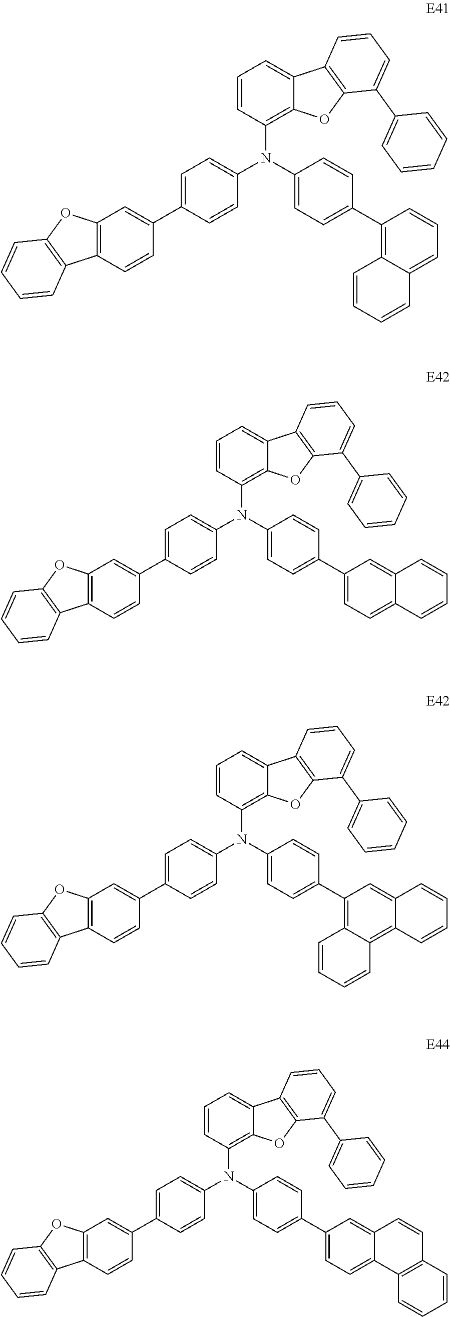 [SMILES: c1ccc(-c2cccc3c2oc2c(N(c4ccc(-c5ccc6c(c5)oc5ccccc56)cc4)c4ccc(-c5cccc6ccccc56)cc4)cccc23)cc1 ; c1ccc(-c2cccc3c2oc2c(N(c4ccc(-c5ccc6ccccc6c5)cc4)c4ccc(-c5ccc6c(c5)oc5ccccc56)cc4)cccc23)cc1 ; c1ccc(-c2cccc3c2oc2c(N(c4ccc(-c5ccc6c(c5)oc5ccccc56)cc4)c4ccc(-c5cc6ccccc6c6ccccc56)cc4)cccc23)cc1 ; c1ccc(-c2cccc3c2oc2c(N(c4ccc(-c5ccc6c(ccc7ccccc76)c5)cc4)c4ccc(-c5ccc6c(c5)oc5ccccc56)cc4)cccc23)cc1]

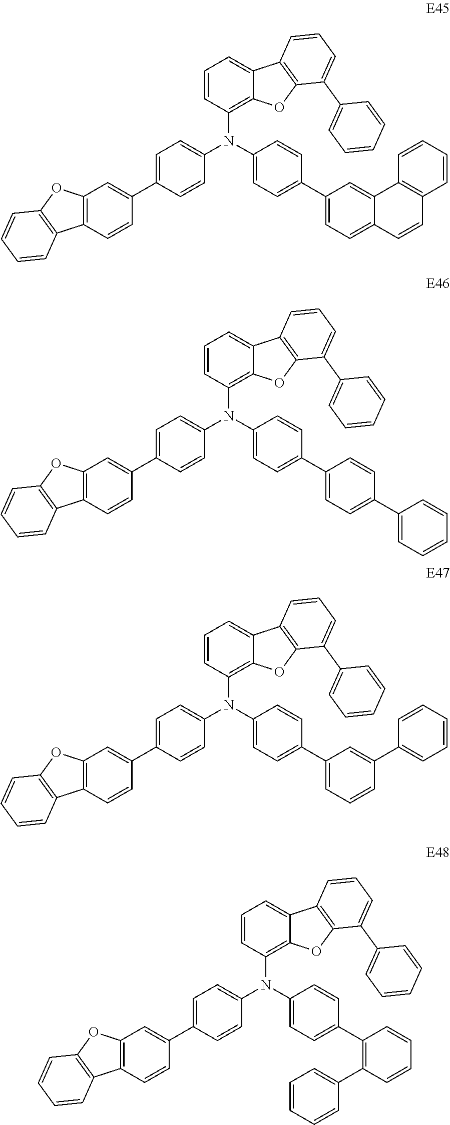 [SMILES: c1ccc(-c2cccc3c2oc2c(N(c4ccc(-c5ccc6c(c5)oc5ccccc56)cc4)c4ccc(-c5ccc6ccc7ccccc7c6c5)cc4)cccc23)cc1 ; c1ccc(-c2ccc(-c3ccc(N(c4ccc(-c5ccc6c(c5)oc5ccccc56)cc4)c4cccc5c4oc4c(-c6ccccc6)cccc45)cc3)cc2)cc1 ; c1ccc(-c2cccc(-c3ccc(N(c4ccc(-c5ccc6c(c5)oc5ccccc56)cc4)c4cccc5c4oc4c(-c6ccccc6)cccc45)cc3)c2)cc1 ; c1ccc(-c2ccccc2-c2ccc(N(c3ccc(-c4ccc5c(c4)oc4ccccc45)cc3)c3cccc4c3oc3c(-c5ccccc5)cccc34)cc2)cc1]

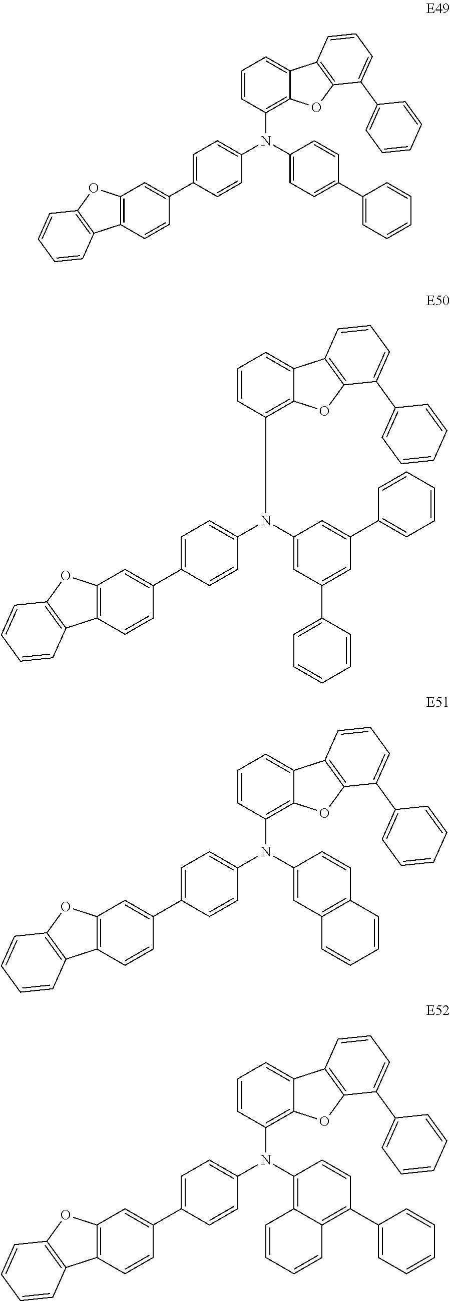 [SMILES: c1ccc(-c2ccc(N(c3ccc(-c4ccc5c(c4)oc4ccccc45)cc3)c3cccc4c3oc3c(-c5ccccc5)cccc34)cc2)cc1 ; c1ccc(-c2cc(-c3ccccc3)cc(N(c3ccc(-c4ccc5c(c4)oc4ccccc45)cc3)c3cccc4c3oc3c(-c5ccccc5)cccc34)c2)cc1 ; c1ccc(-c2cccc3c2oc2c(N(c4ccc(-c5ccc6c(c5)oc5ccccc56)cc4)c4ccc5ccccc5c4)cccc23)cc1 ; c1ccc(-c2ccc(N(c3ccc(-c4ccc5c(c4)oc4ccccc45)cc3)c3cccc4c3oc3c(-c5ccccc5)cccc34)c3ccccc23)cc1]

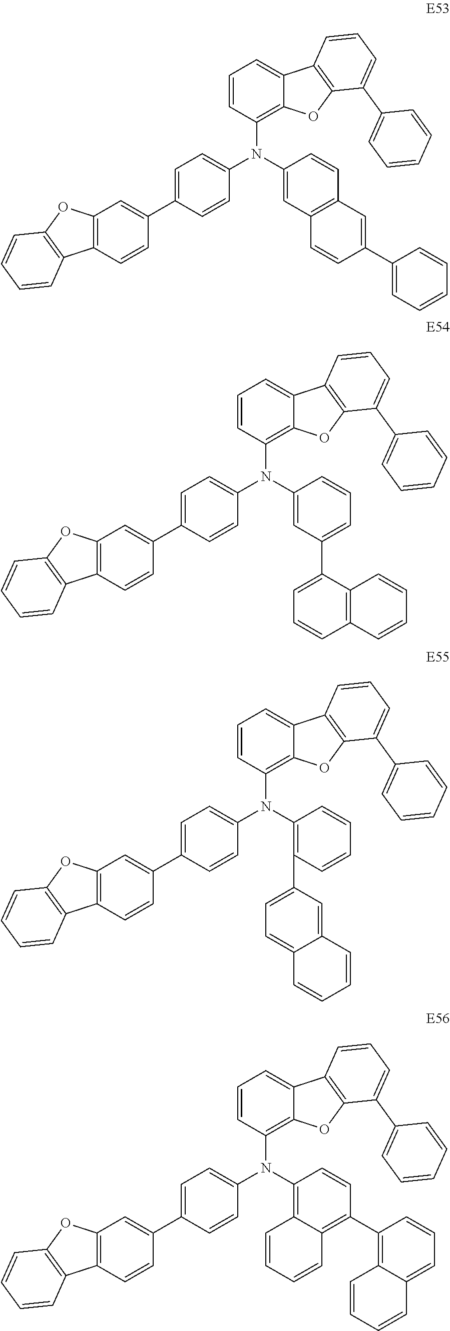

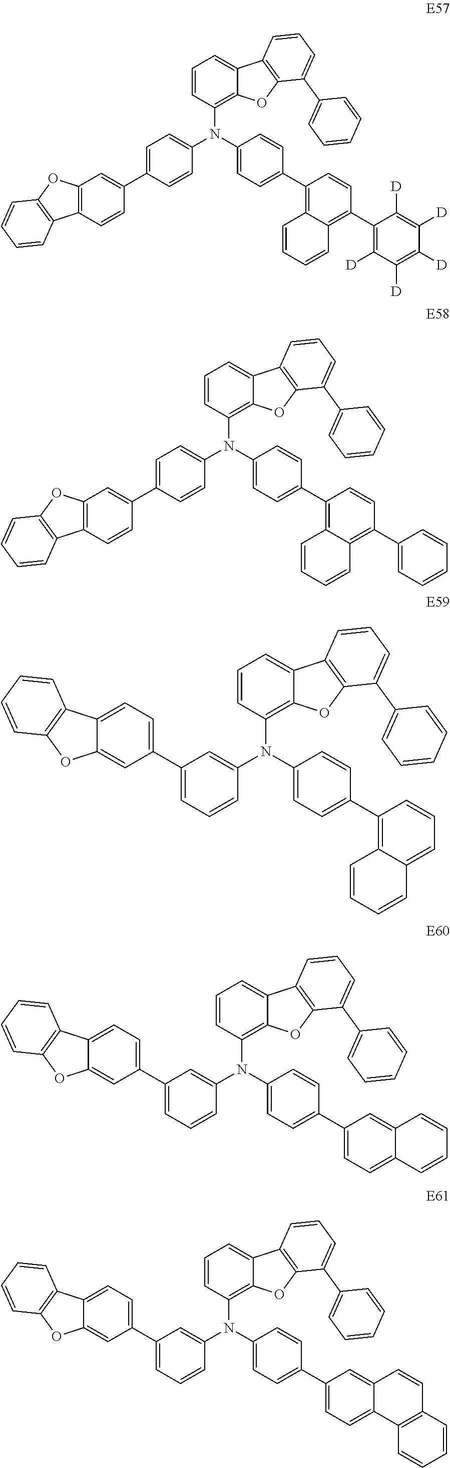

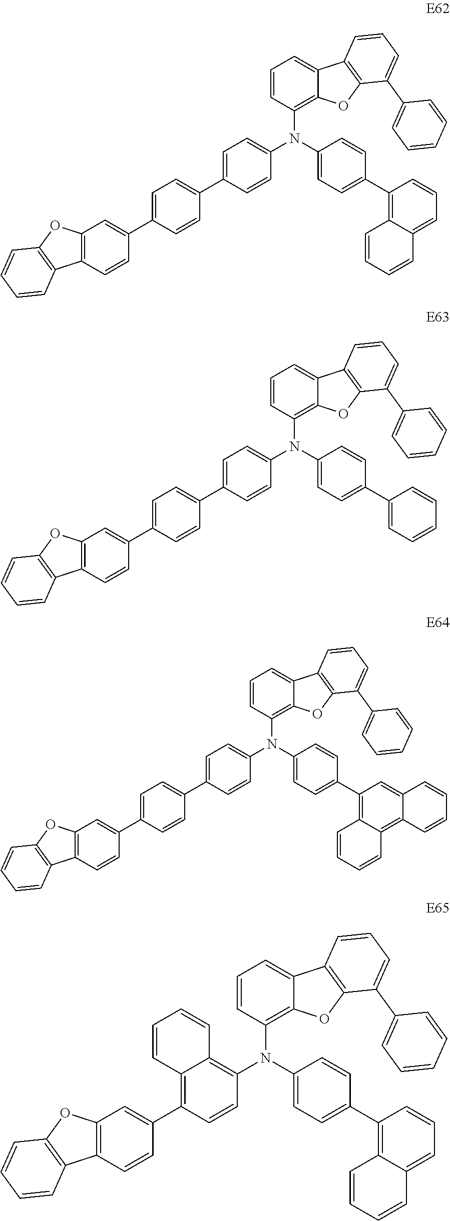 [SMILES: c1ccc(-c2cccc3c2oc2c(N(c4ccc(-c5ccc(-c6ccc7c(c6)oc6ccccc67)cc5)cc4)c4ccc(-c5cccc6ccccc56)cc4)cccc23)cc1 ; c1ccc(-c2ccc(N(c3ccc(-c4ccc(-c5ccc6c(c5)oc5ccccc56)cc4)cc3)c3cccc4c3oc3c(-c5ccccc5)cccc34)cc2)cc1 ; c1ccc(-c2cccc3c2oc2c(N(c4ccc(-c5ccc(-c6ccc7c(c6)oc6ccccc67)cc5)cc4)c4ccc(-c5cc6ccccc6c6ccccc56)cc4)cccc23)cc1 ; c1ccc(-c2cccc3c2oc2c(N(c4ccc(-c5cccc6ccccc56)cc4)c4ccc(-c5ccc6c(c5)oc5ccccc56)c5ccccc45)cccc23)cc1]

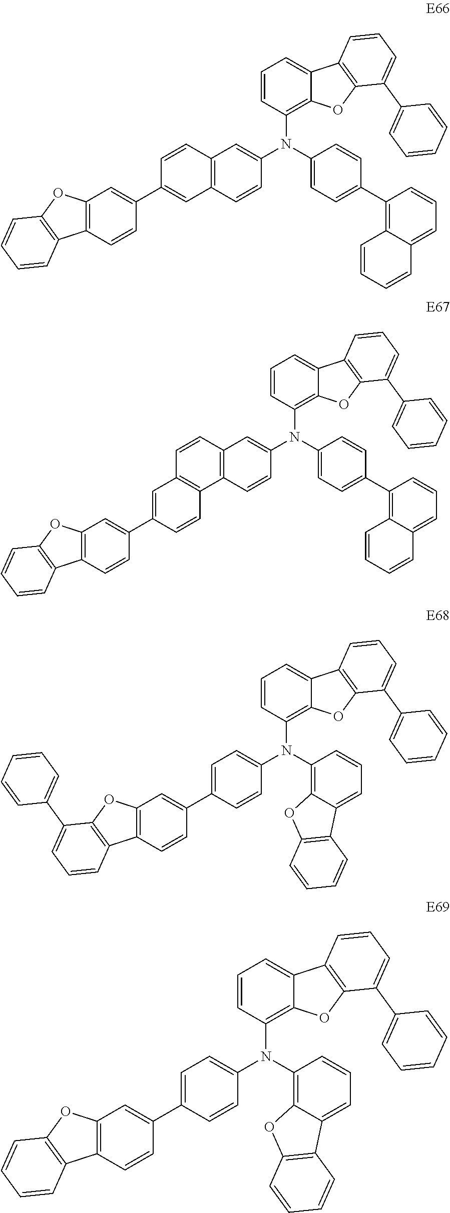 [SMILES: c1ccc(-c2cccc3c2oc2c(N(c4ccc(-c5cccc6ccccc56)cc4)c4ccc5cc(-c6ccc7c(c6)oc6ccccc67)ccc5c4)cccc23)cc1 ; c1ccc(-c2cccc3c2oc2c(N(c4ccc(-c5cccc6ccccc56)cc4)c4ccc5c(ccc6cc(-c7ccc8c(c7)oc7ccccc78)ccc65)c4)cccc23)cc1 ; c1ccc(-c2cccc3c2oc2cc(-c4ccc(N(c5cccc6c5oc5ccccc56)c5cccc6c5oc5c(-c7ccccc7)cccc56)cc4)ccc23)cc1 ; c1ccc(-c2cccc3c2oc2c(N(c4ccc(-c5ccc6c(c5)oc5ccccc56)cc4)c4cccc5c4oc4ccccc45)cccc23)cc1]

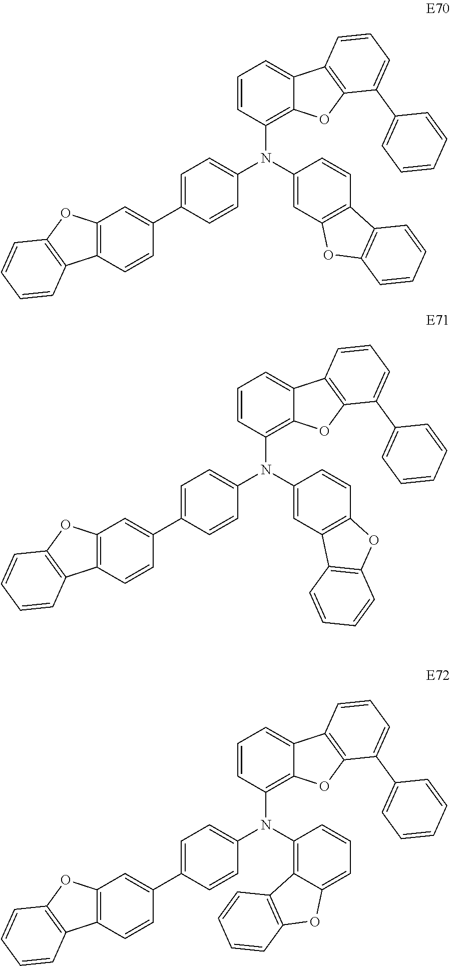 [SMILES: c1ccc(-c2cccc3c2oc2c(N(c4ccc(-c5ccc6c(c5)oc5ccccc56)cc4)c4ccc5c(c4)oc4ccccc45)cccc23)cc1 ; c1ccc(-c2cccc3c2oc2c(N(c4ccc(-c5ccc6c(c5)oc5ccccc56)cc4)c4ccc5oc6ccccc6c5c4)cccc23)cc1 ; c1ccc(-c2cccc3c2oc2c(N(c4ccc(-c5ccc6c(c5)oc5ccccc56)cc4)c4cccc5oc6ccccc6c45)cccc23)cc1]

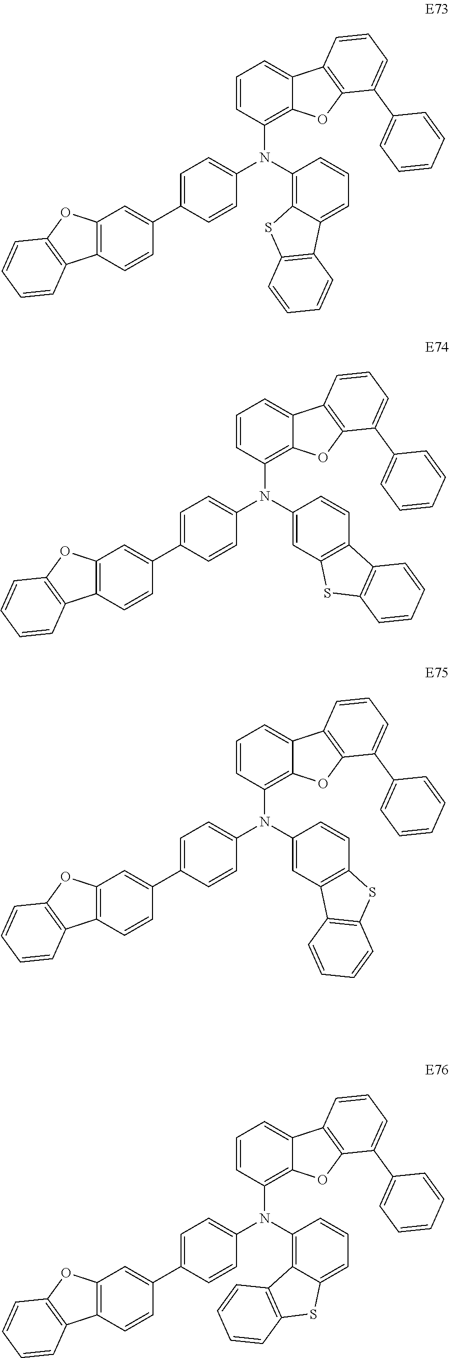 [SMILES: c1ccc(-c2cccc3c2oc2c(N(c4ccc(-c5ccc6c(c5)oc5ccccc56)cc4)c4cccc5c4sc4ccccc45)cccc23)cc1 ; c1ccc(-c2cccc3c2oc2c(N(c4ccc(-c5ccc6c(c5)oc5ccccc56)cc4)c4ccc5c(c4)sc4ccccc45)cccc23)cc1 ; c1ccc(-c2cccc3c2oc2c(N(c4ccc(-c5ccc6c(c5)oc5ccccc56)cc4)c4ccc5sc6ccccc6c5c4)cccc23)cc1 ; c1ccc(-c2cccc3c2oc2c(N(c4ccc(-c5ccc6c(c5)oc5ccccc56)cc4)c4cccc5sc6ccccc6c45)cccc23)cc1]

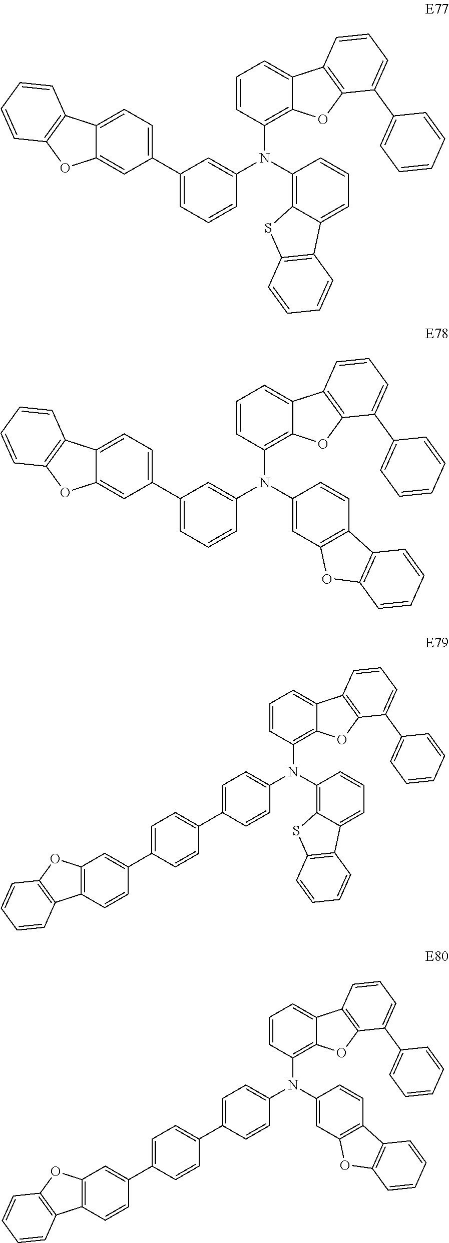 [SMILES: c1ccc(-c2cccc3c2oc2c(N(c4cccc(-c5ccc6c(c5)oc5ccccc56)c4)c4cccc5c4sc4ccccc45)cccc23)cc1 ; c1ccc(-c2cccc3c2oc2c(N(c4cccc(-c5ccc6c(c5)oc5ccccc56)c4)c4ccc5c(c4)oc4ccccc45)cccc23)cc1 ; c1ccc(-c2cccc3c2oc2c(N(c4ccc(-c5ccc(-c6ccc7c(c6)oc6ccccc67)cc5)cc4)c4cccc5c4sc4ccccc45)cccc23)cc1 ; c1ccc(-c2cccc3c2oc2c(N(c4ccc(-c5ccc(-c6ccc7c(c6)oc6ccccc67)cc5)cc4)c4ccc5c(c4)oc4ccccc45)cccc23)cc1]

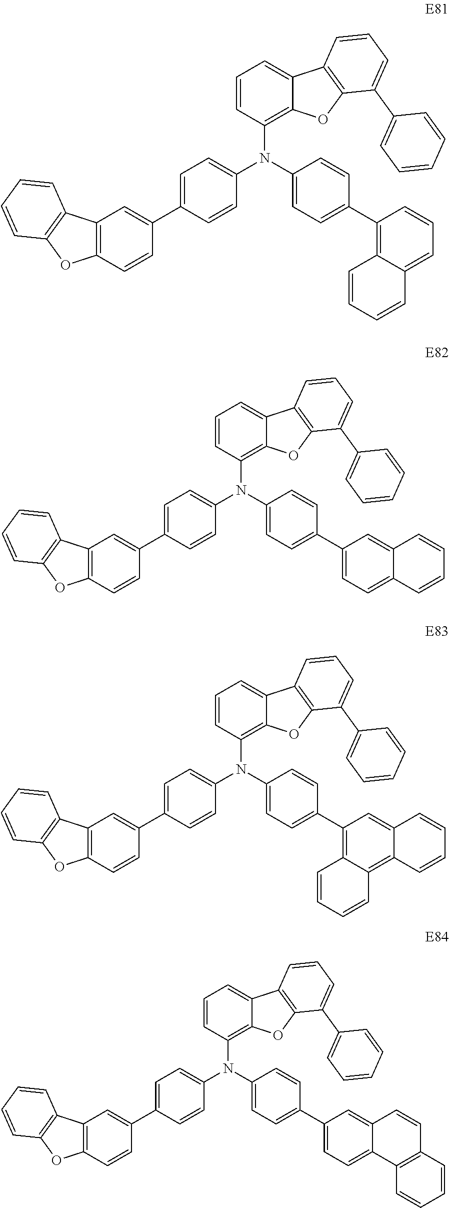 [SMILES: c1ccc(-c2cccc3c2oc2c(N(c4ccc(-c5ccc6oc7ccccc7c6c5)cc4)c4ccc(-c5cccc6ccccc56)cc4)cccc23)cc1 ; c1ccc(-c2cccc3c2oc2c(N(c4ccc(-c5ccc6ccccc6c5)cc4)c4ccc(-c5ccc6oc7ccccc7c6c5)cc4)cccc23)cc1 ; c1ccc(-c2cccc3c2oc2c(N(c4ccc(-c5ccc6oc7ccccc7c6c5)cc4)c4ccc(-c5cc6ccccc6c6ccccc56)cc4)cccc23)cc1 ; c1ccc(-c2cccc3c2oc2c(N(c4ccc(-c5ccc6c(ccc7ccccc76)c5)cc4)c4ccc(-c5ccc6oc7ccccc7c6c5)cc4)cccc23)cc1]

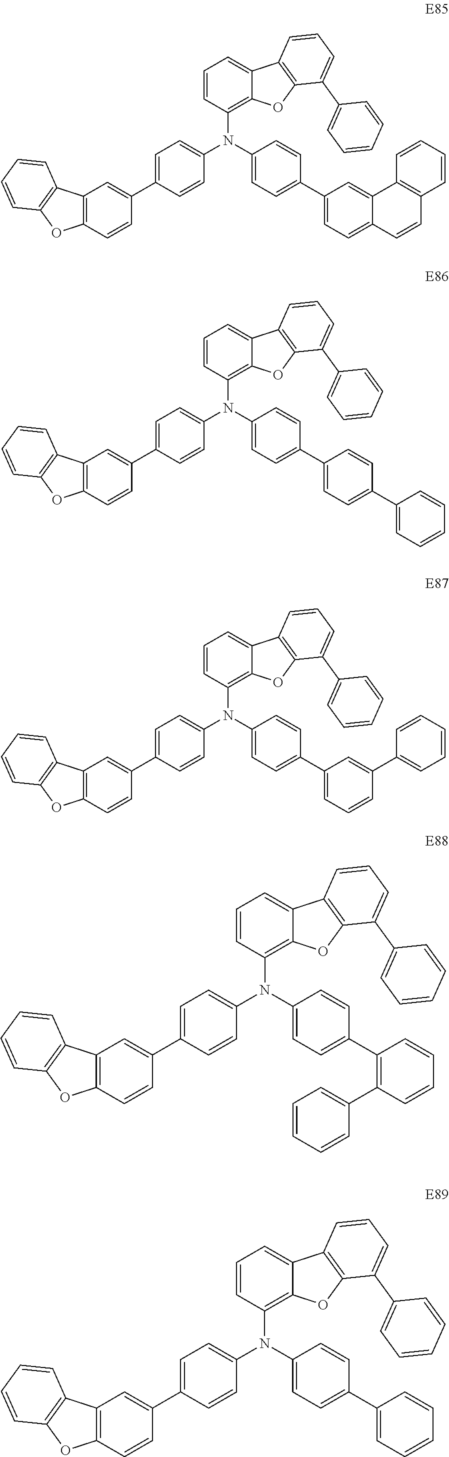 [SMILES: c1ccc(-c2cccc3c2oc2c(N(c4ccc(-c5ccc6ccc7ccccc7c6c5)cc4)c4ccc(-c5ccc6oc7ccccc7c6c5)cc4)cccc23)cc1 ; c1ccc(-c2ccc(-c3ccc(N(c4ccc(-c5ccc6oc7ccccc7c6c5)cc4)c4cccc5c4oc4c(-c6ccccc6)cccc45)cc3)cc2)cc1 ; c1ccc(-c2cccc(-c3ccc(N(c4ccc(-c5ccc6oc7ccccc7c6c5)cc4)c4cccc5c4oc4c(-c6ccccc6)cccc45)cc3)c2)cc1 ; c1ccc(-c2ccccc2-c2ccc(N(c3ccc(-c4ccc5oc6ccccc6c5c4)cc3)c3cccc4c3oc3c(-c5ccccc5)cccc34)cc2)cc1 ; c1ccc(-c2ccc(N(c3ccc(-c4ccc5oc6ccccc6c5c4)cc3)c3cccc4c3oc3c(-c5ccccc5)cccc34)cc2)cc1]

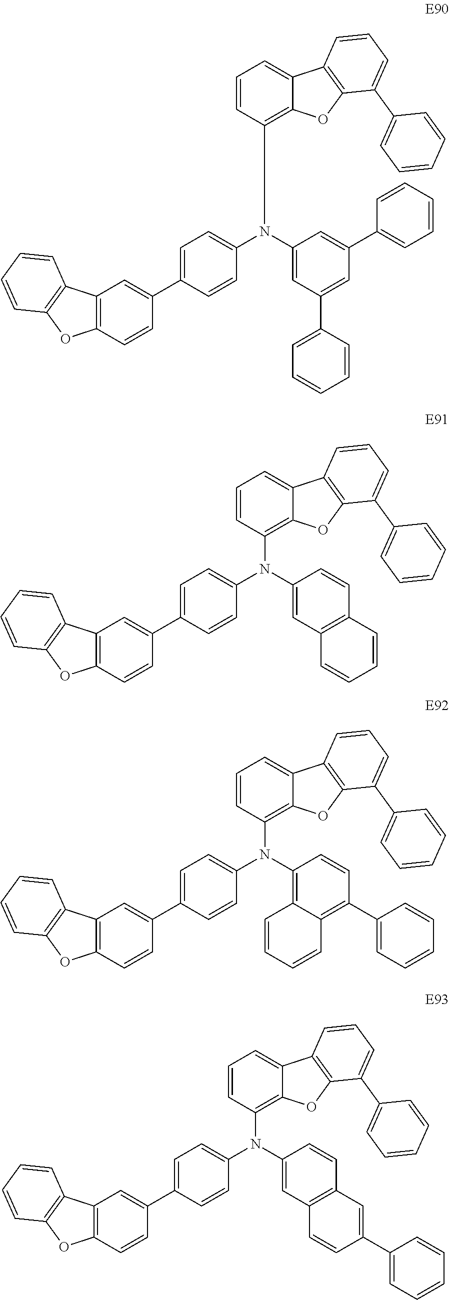 [SMILES: c1ccc(-c2cc(-c3ccccc3)cc(N(c3ccc(-c4ccc5oc6ccccc6c5c4)cc3)c3cccc4c3oc3c(-c5ccccc5)cccc34)c2)cc1 ; c1ccc(-c2cccc3c2oc2c(N(c4ccc(-c5ccc6oc7ccccc7c6c5)cc4)c4ccc5ccccc5c4)cccc23)cc1 ; c1ccc(-c2ccc(N(c3ccc(-c4ccc5oc6ccccc6c5c4)cc3)c3cccc4c3oc3c(-c5ccccc5)cccc34)c3ccccc23)cc1 ; c1ccc(-c2ccc3cc(N(c4ccc(-c5ccc6oc7ccccc7c6c5)cc4)c4cccc5c4oc4c(-c6ccccc6)cccc45)ccc3c2)cc1]

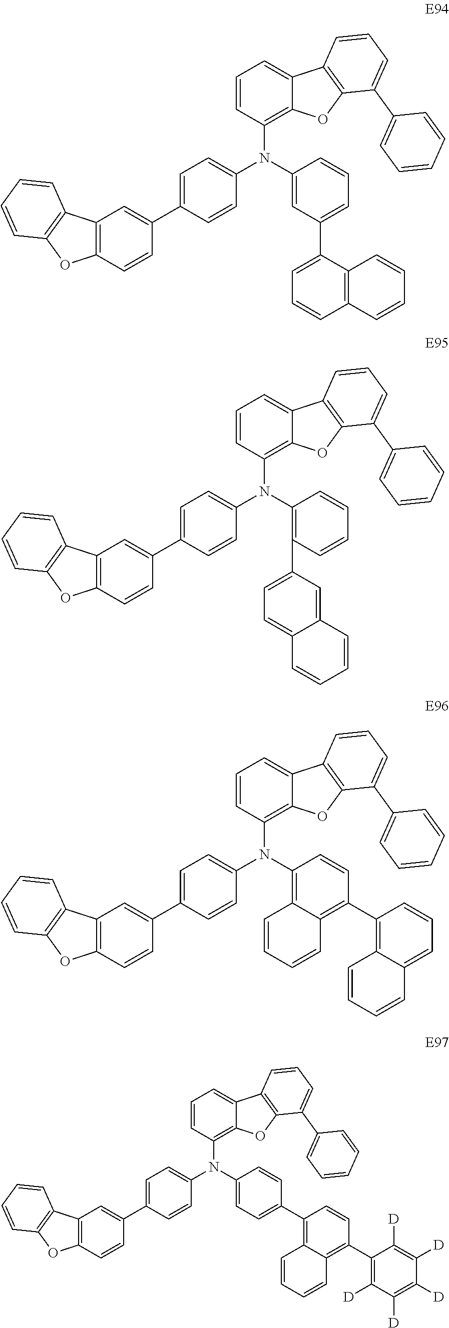 [SMILES: c1ccc(-c2cccc3c2oc2c(N(c4ccc(-c5ccc6oc7ccccc7c6c5)cc4)c4cccc(-c5cccc6ccccc56)c4)cccc23)cc1 ; c1ccc(-c2cccc3c2oc2c(N(c4ccc(-c5ccc6oc7ccccc7c6c5)cc4)c4ccccc4-c4ccc5ccccc5c4)cccc23)cc1 ; c1ccc(-c2cccc3c2oc2c(N(c4ccc(-c5ccc6oc7ccccc7c6c5)cc4)c4ccc(-c5cccc6ccccc56)c5ccccc45)cccc23)cc1 ; [2H]c1c([2H])c([2H])c(-c2ccc(-c3ccc(N(c4ccc(-c5ccc6oc7ccccc7c6c5)cc4)c4cccc5c4oc4c(-c6ccccc6)cccc45)cc3)c3ccccc23)c([2H])c1[2H]]

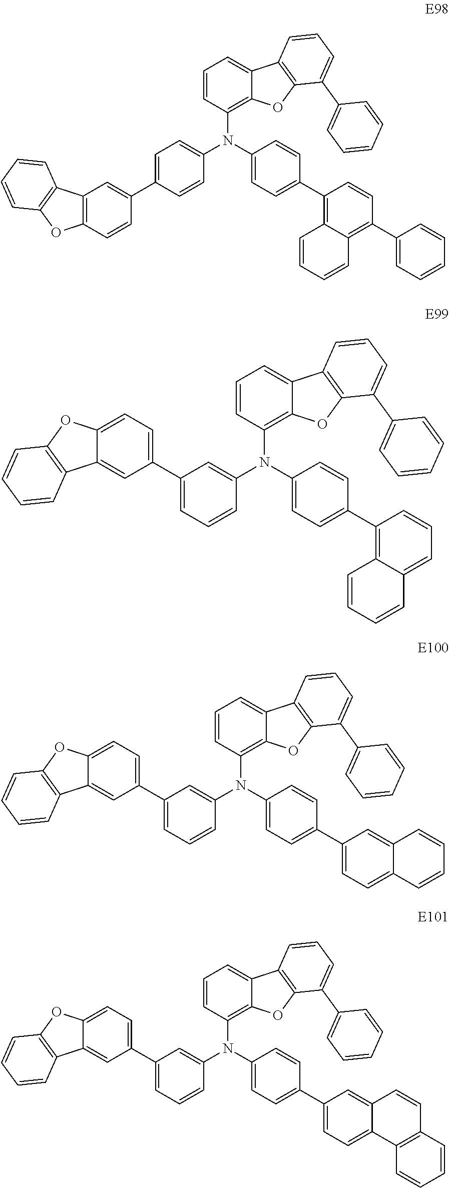 [SMILES: c1ccc(-c2ccc(-c3ccc(N(c4ccc(-c5ccc6oc7ccccc7c6c5)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5ccc6oc7ccccc7c6c5)c4)cccc23)cc1 ; c1ccc(-c2cccc3c2oc2c(N(c4ccc(-c5ccc6ccccc6c5)cc4)c4cccc(-c5ccc6oc7ccccc7c6c5)c4)cccc23)cc1 ; c1ccc(-c2cccc3c2oc2c(N(c4ccc(-c5ccc6c(ccc7ccccc76)c5)cc4)c4cccc(-c5ccc6oc7ccccc7c6c5)c4)cccc23)cc1]

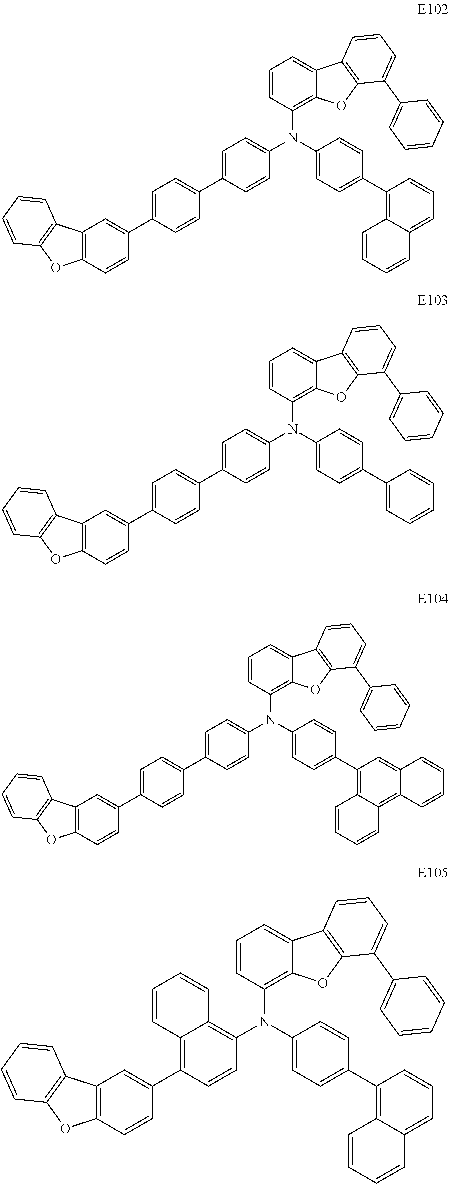 [SMILES: c1ccc(-c2cccc3c2oc2c(N(c4ccc(-c5ccc(-c6ccc7oc8ccccc8c7c6)cc5)cc4)c4ccc(-c5cccc6ccccc56)cc4)cccc23)cc1 ; c1ccc(-c2ccc(N(c3ccc(-c4ccc(-c5ccc6oc7ccccc7c6c5)cc4)cc3)c3cccc4c3oc3c(-c5ccccc5)cccc34)cc2)cc1 ; c1ccc(-c2cccc3c2oc2c(N(c4ccc(-c5ccc(-c6ccc7oc8ccccc8c7c6)cc5)cc4)c4ccc(-c5cc6ccccc6c6ccccc56)cc4)cccc23)cc1 ; c1ccc(-c2cccc3c2oc2c(N(c4ccc(-c5cccc6ccccc56)cc4)c4ccc(-c5ccc6oc7ccccc7c6c5)c5ccccc45)cccc23)cc1]

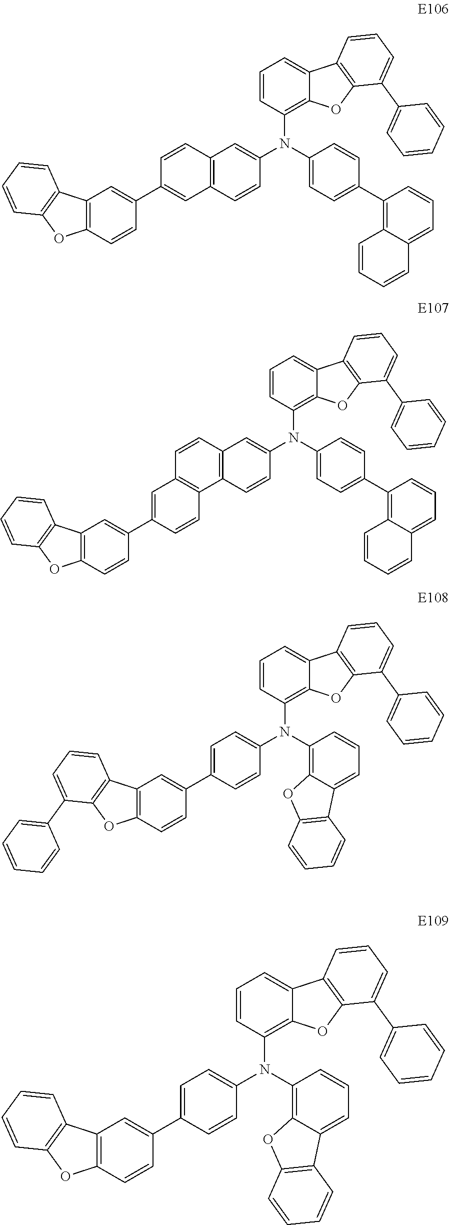 [SMILES: c1ccc(-c2cccc3c2oc2c(N(c4ccc(-c5cccc6ccccc56)cc4)c4ccc5cc(-c6ccc7oc8ccccc8c7c6)ccc5c4)cccc23)cc1 ; c1ccc(-c2cccc3c2oc2c(N(c4ccc(-c5cccc6ccccc56)cc4)c4ccc5c(ccc6cc(-c7ccc8oc9ccccc9c8c7)ccc65)c4)cccc23)cc1 ; c1ccc(-c2cccc3c2oc2ccc(-c4ccc(N(c5cccc6c5oc5ccccc56)c5cccc6c5oc5c(-c7ccccc7)cccc56)cc4)cc23)cc1 ; c1ccc(-c2cccc3c2oc2c(N(c4ccc(-c5ccc6oc7ccccc7c6c5)cc4)c4cccc5c4oc4ccccc45)cccc23)cc1]

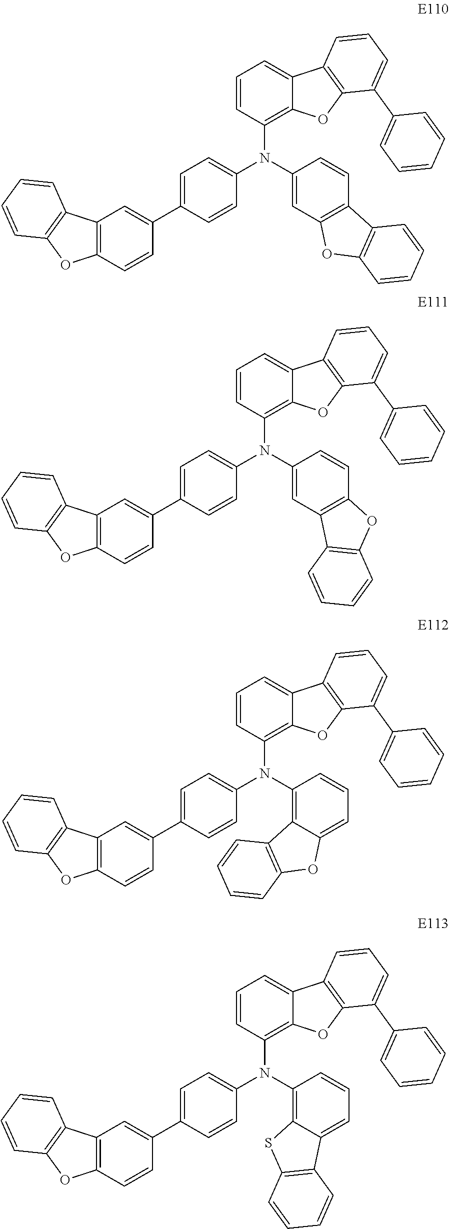

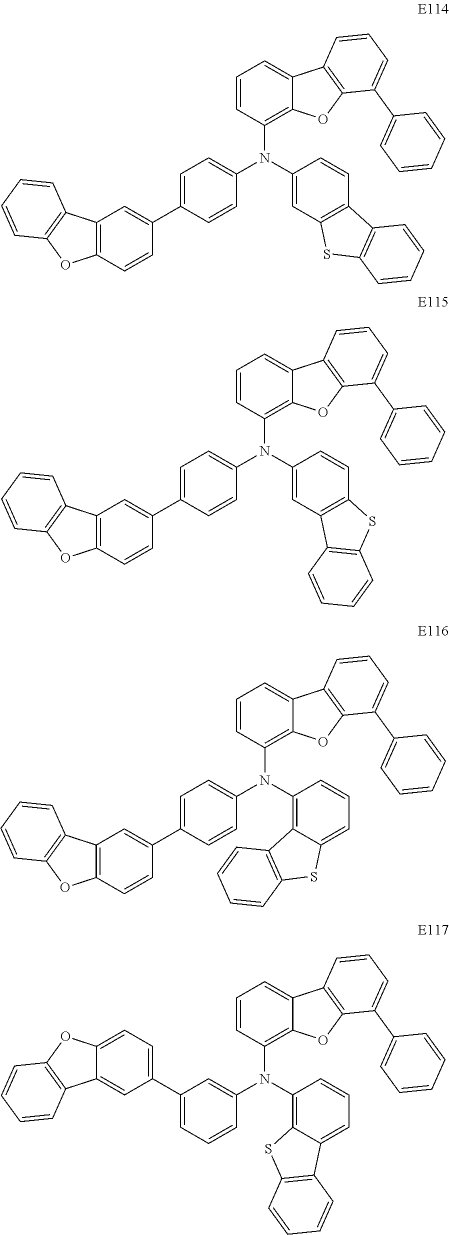

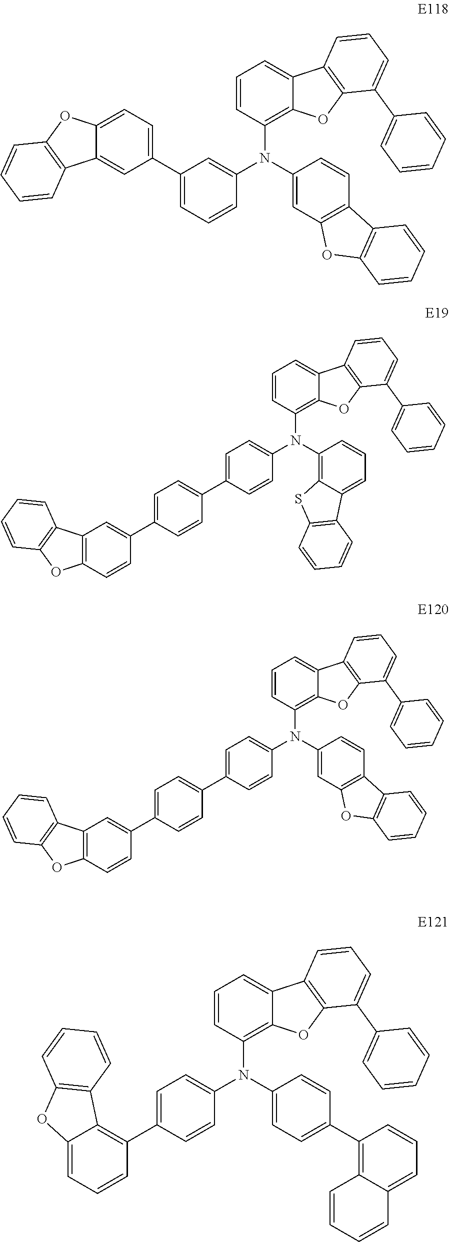 [SMILES: c1ccc(-c2cccc3c2oc2c(N(c4cccc(-c5ccc6oc7ccccc7c6c5)c4)c4ccc5c(c4)oc4ccccc45)cccc23)cc1 ; c1ccc(-c2cccc3c2oc2c(N(c4ccc(-c5ccc(-c6ccc7oc8ccccc8c7c6)cc5)cc4)c4cccc5c4sc4ccccc45)cccc23)cc1 ; c1ccc(-c2cccc3c2oc2c(N(c4ccc(-c5ccc(-c6ccc7oc8ccccc8c7c6)cc5)cc4)c4ccc5c(c4)oc4ccccc45)cccc23)cc1 ; c1ccc(-c2cccc3c2oc2c(N(c4ccc(-c5cccc6ccccc56)cc4)c4ccc(-c5cccc6oc7ccccc7c56)cc4)cccc23)cc1]

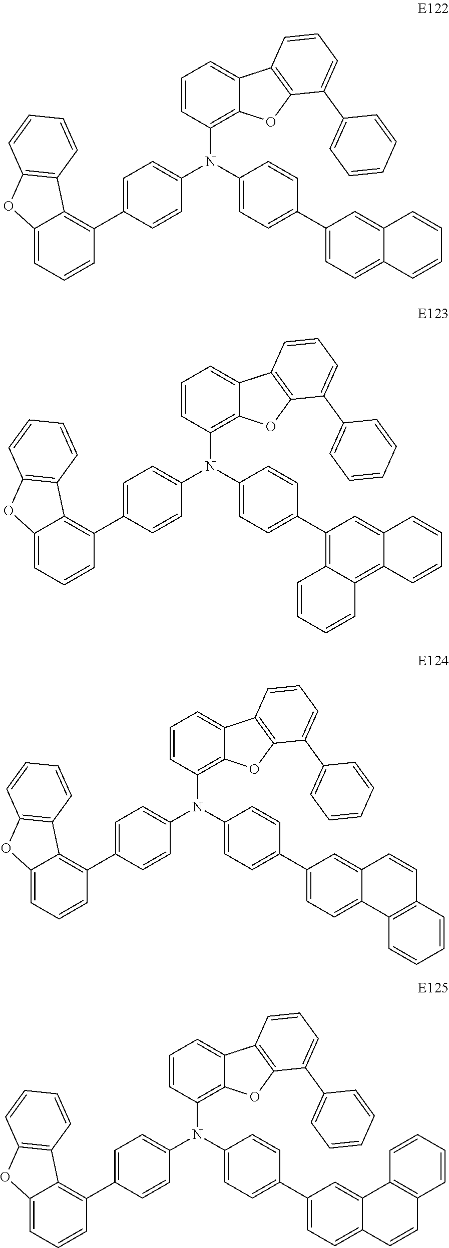 [SMILES: c1ccc(-c2cccc3c2oc2c(N(c4ccc(-c5ccc6ccccc6c5)cc4)c4ccc(-c5cccc6oc7ccccc7c56)cc4)cccc23)cc1 ; c1ccc(-c2cccc3c2oc2c(N(c4ccc(-c5cc6ccccc6c6ccccc56)cc4)c4ccc(-c5cccc6oc7ccccc7c56)cc4)cccc23)cc1 ; c1ccc(-c2cccc3c2oc2c(N(c4ccc(-c5ccc6c(ccc7ccccc76)c5)cc4)c4ccc(-c5cccc6oc7ccccc7c56)cc4)cccc23)cc1 ; c1ccc(-c2cccc3c2oc2c(N(c4ccc(-c5ccc6ccc7ccccc7c6c5)cc4)c4ccc(-c5cccc6oc7ccccc7c56)cc4)cccc23)cc1]

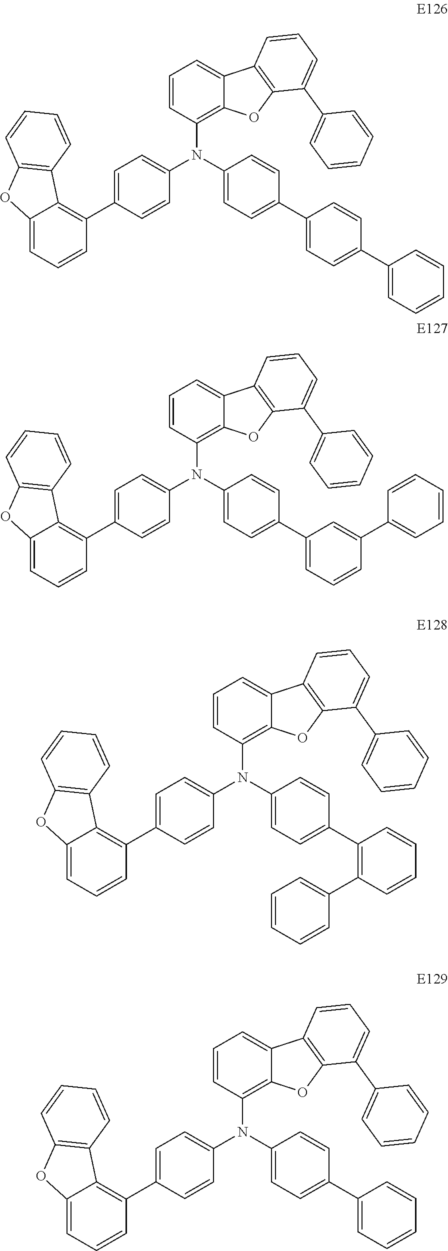 [SMILES: c1ccc(-c2ccc(-c3ccc(N(c4ccc(-c5cccc6oc7ccccc7c56)cc4)c4cccc5c4oc4c(-c6ccccc6)cccc45)cc3)cc2)cc1 ; c1ccc(-c2cccc(-c3ccc(N(c4ccc(-c5cccc6oc7ccccc7c56)cc4)c4cccc5c4oc4c(-c6ccccc6)cccc45)cc3)c2)cc1 ; c1ccc(-c2ccccc2-c2ccc(N(c3ccc(-c4cccc5oc6ccccc6c45)cc3)c3cccc4c3oc3c(-c5ccccc5)cccc34)cc2)cc1 ; c1ccc(-c2ccc(N(c3ccc(-c4cccc5oc6ccccc6c45)cc3)c3cccc4c3oc3c(-c5ccccc5)cccc34)cc2)cc1]

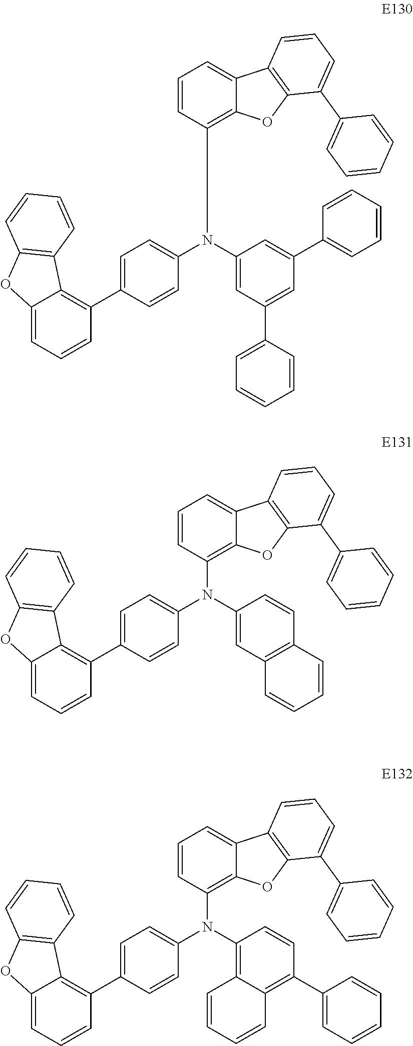 [SMILES: c1ccc(-c2cc(-c3ccccc3)cc(N(c3ccc(-c4cccc5oc6ccccc6c45)cc3)c3cccc4c3oc3c(-c5ccccc5)cccc34)c2)cc1 ; c1ccc(-c2cccc3c2oc2c(N(c4ccc(-c5cccc6oc7ccccc7c56)cc4)c4ccc5ccccc5c4)cccc23)cc1 ; c1ccc(-c2ccc(N(c3ccc(-c4cccc5oc6ccccc6c45)cc3)c3cccc4c3oc3c(-c5ccccc5)cccc34)c3ccccc23)cc1]

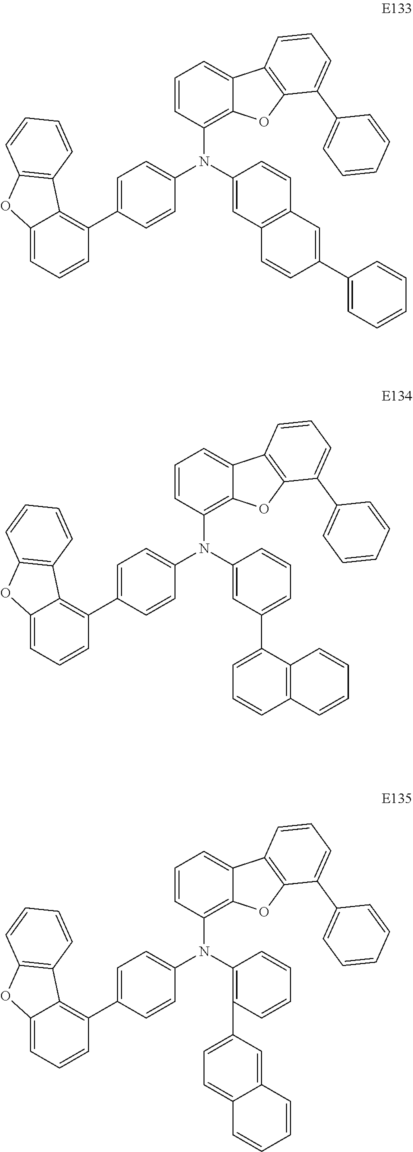 [SMILES: c1ccc(-c2ccc3cc(N(c4ccc(-c5cccc6oc7ccccc7c56)cc4)c4cccc5c4oc4c(-c6ccccc6)cccc45)ccc3c2)cc1 ; c1ccc(-c2cccc3c2oc2c(N(c4ccc(-c5cccc6oc7ccccc7c56)cc4)c4cccc(-c5cccc6ccccc56)c4)cccc23)cc1 ; c1ccc(-c2cccc3c2oc2c(N(c4ccc(-c5cccc6oc7ccccc7c56)cc4)c4ccccc4-c4ccc5ccccc5c4)cccc23)cc1]

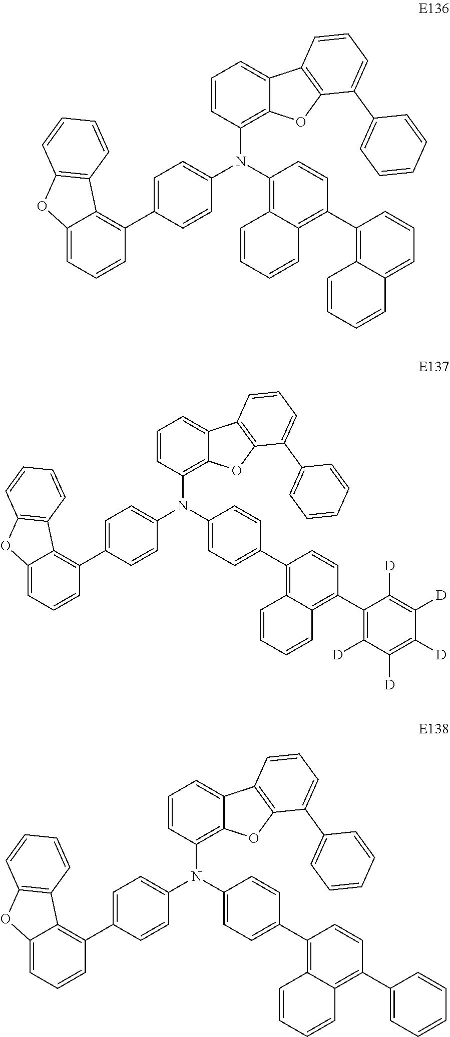 [SMILES: c1ccc(-c2cccc3c2oc2c(N(c4ccc(-c5cccc6oc7ccccc7c56)cc4)c4ccc(-c5cccc6ccccc56)c5ccccc45)cccc23)cc1 ; [2H]c1c([2H])c([2H])c(-c2ccc(-c3ccc(N(c4ccc(-c5cccc6oc7ccccc7c56)cc4)c4cccc5c4oc4c(-c6ccccc6)cccc45)cc3)c3ccccc23)c([2H])c1[2H] ; c1ccc(-c2ccc(-c3ccc(N(c4ccc(-c5cccc6oc7ccccc7c56)cc4)c4cccc5c4oc4c(-c6ccccc6)cccc45)cc3)c3ccccc23)cc1]

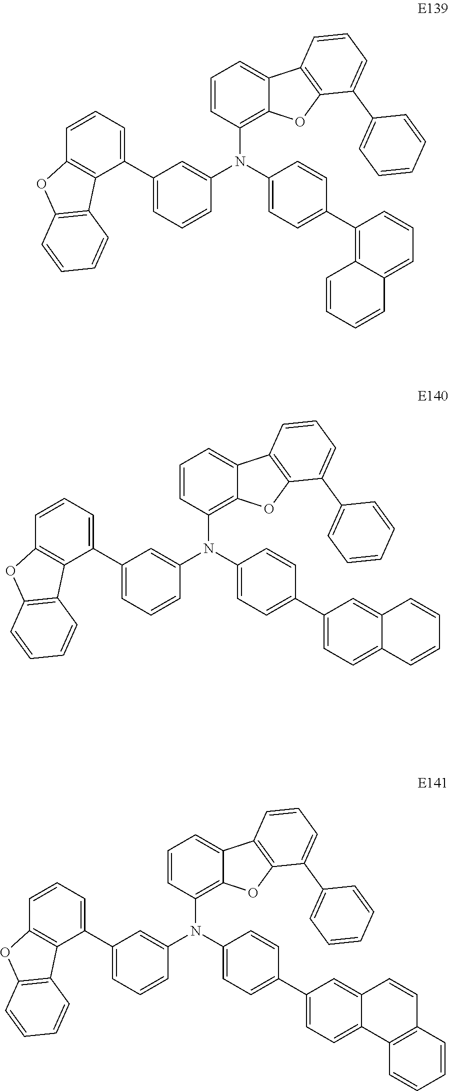 [SMILES: c1ccc(-c2cccc3c2oc2c(N(c4ccc(-c5cccc6ccccc56)cc4)c4cccc(-c5cccc6oc7ccccc7c56)c4)cccc23)cc1 ; c1ccc(-c2cccc3c2oc2c(N(c4ccc(-c5ccc6ccccc6c5)cc4)c4cccc(-c5cccc6oc7ccccc7c56)c4)cccc23)cc1 ; c1ccc(-c2cccc3c2oc2c(N(c4ccc(-c5ccc6c(ccc7ccccc76)c5)cc4)c4cccc(-c5cccc6oc7ccccc7c56)c4)cccc23)cc1]

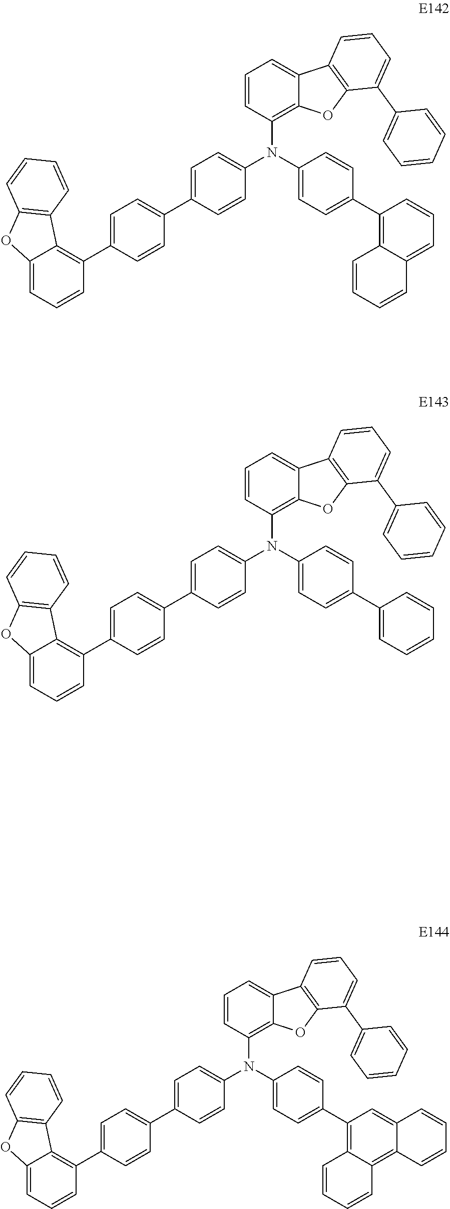 [SMILES: c1ccc(-c2cccc3c2oc2c(N(c4ccc(-c5ccc(-c6cccc7oc8ccccc8c67)cc5)cc4)c4ccc(-c5cccc6ccccc56)cc4)cccc23)cc1 ; c1ccc(-c2ccc(N(c3ccc(-c4ccc(-c5cccc6oc7ccccc7c56)cc4)cc3)c3cccc4c3oc3c(-c5ccccc5)cccc34)cc2)cc1 ; c1ccc(-c2cccc3c2oc2c(N(c4ccc(-c5ccc(-c6cccc7oc8ccccc8c67)cc5)cc4)c4ccc(-c5cc6ccccc6c6ccccc56)cc4)cccc23)cc1]

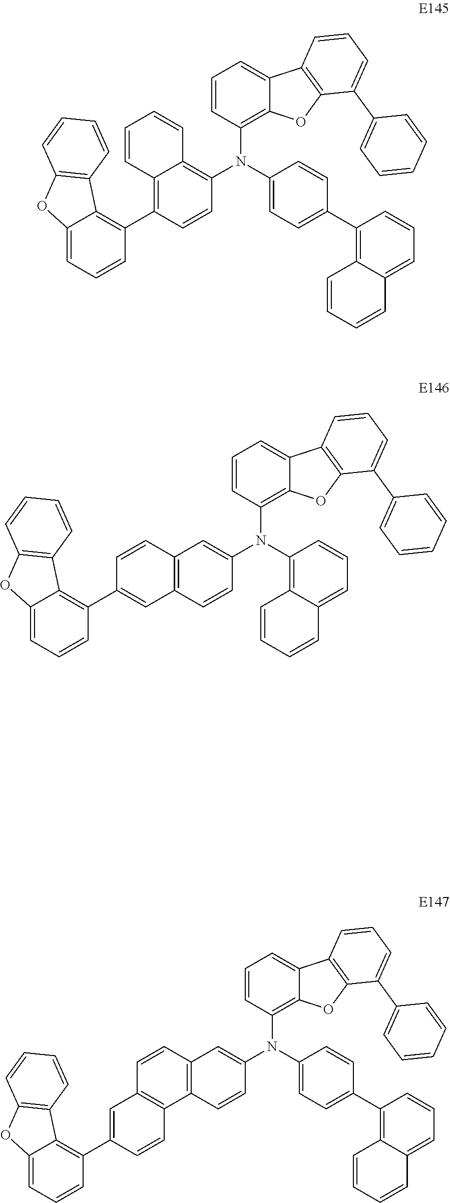 [SMILES: c1ccc(-c2cccc3c2oc2c(N(c4ccc(-c5cccc6ccccc56)cc4)c4ccc(-c5cccc6oc7ccccc7c56)c5ccccc45)cccc23)cc1 ; c1ccc(-c2cccc3c2oc2c(N(c4ccc5cc(-c6cccc7oc8ccccc8c67)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7cccc8oc9ccccc9c78)ccc65)c4)cccc23)cc1]

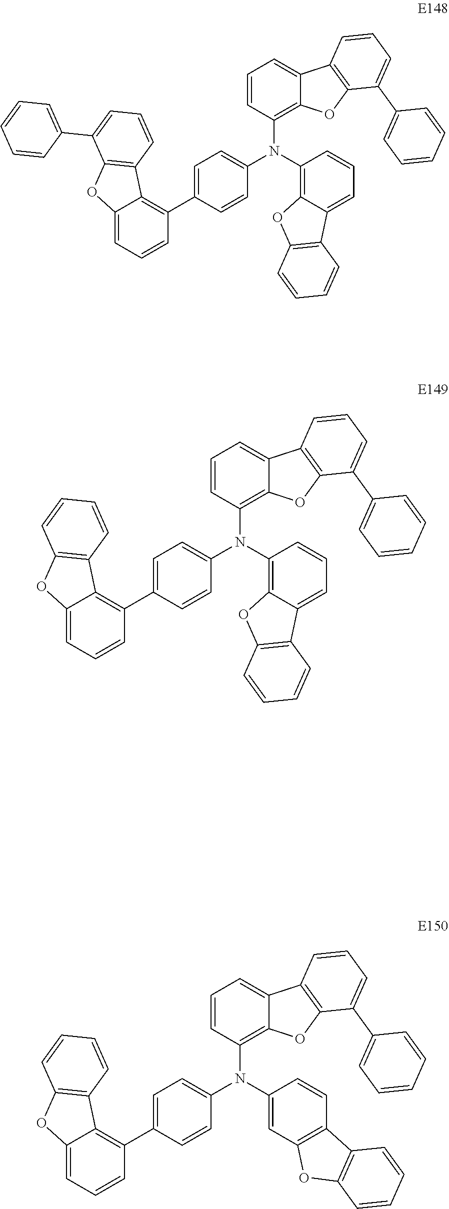 [SMILES: c1ccc(-c2cccc3c2oc2c(N(c4ccc(-c5cccc6oc7c(-c8ccccc8)cccc7c56)cc4)c4cccc5c4oc4ccccc45)cccc23)cc1 ; c1ccc(-c2cccc3c2oc2c(N(c4ccc(-c5cccc6oc7ccccc7c56)cc4)c4cccc5c4oc4ccccc45)cccc23)cc1 ; c1ccc(-c2cccc3c2oc2c(N(c4ccc(-c5cccc6oc7ccccc7c56)cc4)c4ccc5c(c4)oc4ccccc45)cccc23)cc1]

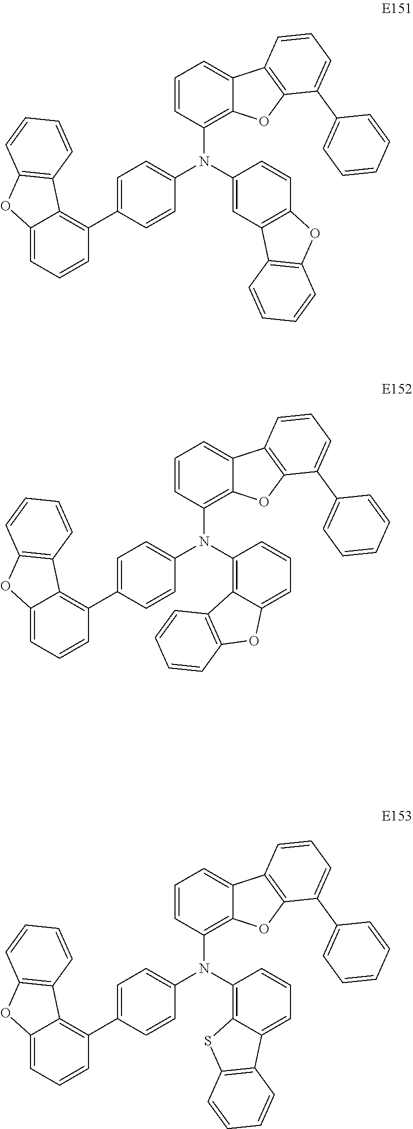 [SMILES: c1ccc(-c2cccc3c2oc2c(N(c4ccc(-c5cccc6oc7ccccc7c56)cc4)c4ccc5oc6ccccc6c5c4)cccc23)cc1 ; c1ccc(-c2cccc3c2oc2c(N(c4ccc(-c5cccc6oc7ccccc7c56)cc4)c4cccc5oc6ccccc6c45)cccc23)cc1 ; c1ccc(-c2cccc3c2oc2c(N(c4ccc(-c5cccc6oc7ccccc7c56)cc4)c4cccc5c4sc4ccccc45)cccc23)cc1]

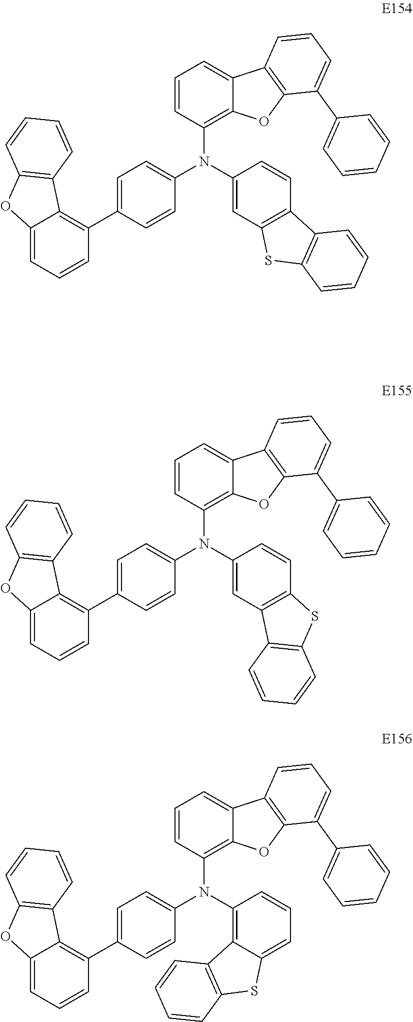 [SMILES: c1ccc(-c2cccc3c2oc2c(N(c4ccc(-c5cccc6oc7ccccc7c56)cc4)c4ccc5c(c4)sc4ccccc45)cccc23)cc1 ; c1ccc(-c2cccc3c2oc2c(N(c4ccc(-c5cccc6oc7ccccc7c56)cc4)c4ccc5sc6ccccc6c5c4)cccc23)cc1 ; c1ccc(-c2cccc3c2oc2c(N(c4ccc(-c5cccc6oc7ccccc7c56)cc4)c4cccc5sc6ccccc6c45)cccc23)cc1]

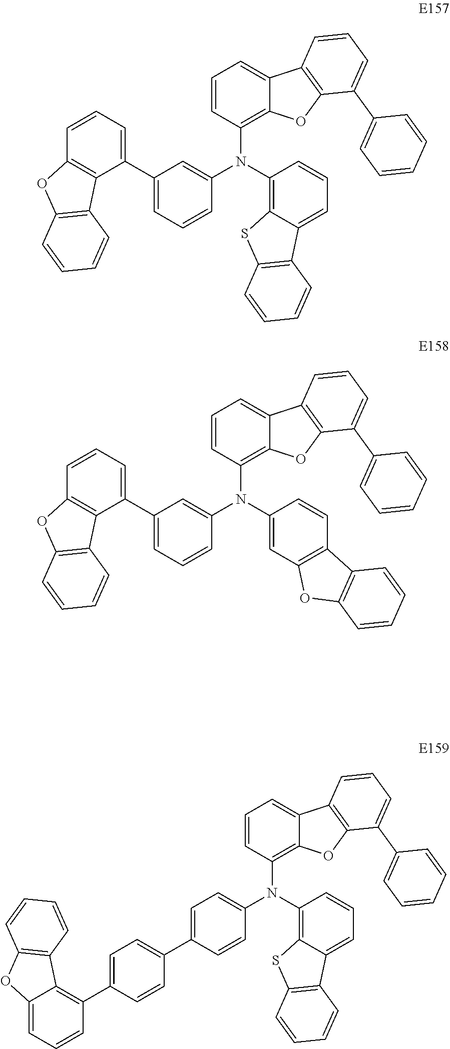 [SMILES: c1ccc(-c2cccc3c2oc2c(N(c4cccc(-c5cccc6oc7ccccc7c56)c4)c4cccc5c4sc4ccccc45)cccc23)cc1 ; c1ccc(-c2cccc3c2oc2c(N(c4cccc(-c5cccc6oc7ccccc7c56)c4)c4ccc5c(c4)oc4ccccc45)cccc23)cc1 ; c1ccc(-c2cccc3c2oc2c(N(c4ccc(-c5ccc(-c6cccc7oc8ccccc8c67)cc5)cc4)c4cccc5c4sc4ccccc45)cccc23)cc1]

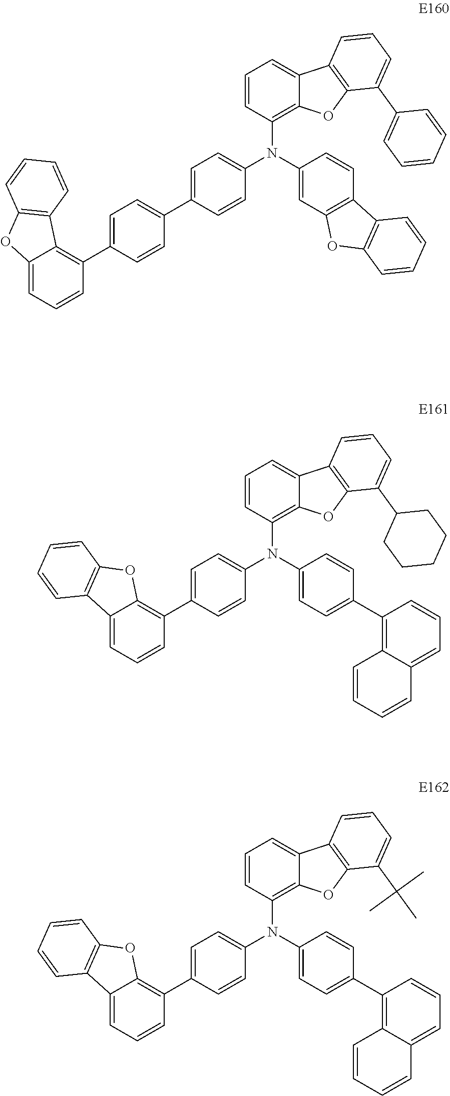 [SMILES: c1ccc(-c2cccc3c2oc2c(N(c4ccc(-c5ccc(-c6cccc7oc8ccccc8c67)cc5)cc4)c4ccc5c(c4)oc4ccccc45)cccc23)cc1 ; c1ccc2c(-c3ccc(N(c4ccc(-c5cccc6c5oc5ccccc56)cc4)c4cccc5c4oc4c(C6CCCCC6)cccc45)cc3)cccc2c1 ; CC(C)(C)c1cccc2c1oc1c(N(c3ccc(-c4cccc5ccccc45)cc3)c3ccc(-c4cccc5c4oc4ccccc45)cc3)cccc12]

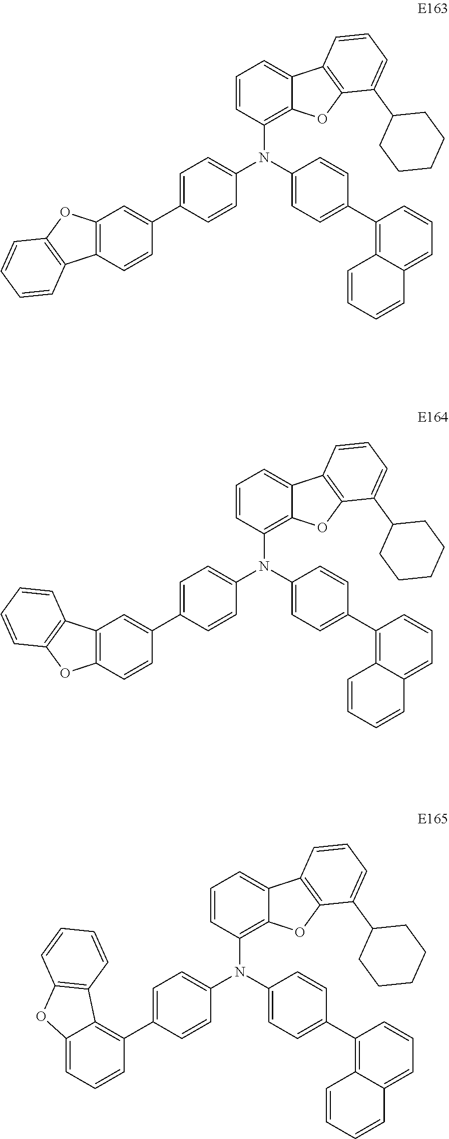 [SMILES: c1ccc2c(-c3ccc(N(c4ccc(-c5ccc6c(c5)oc5ccccc56)cc4)c4cccc5c4oc4c(C6CCCCC6)cccc45)cc3)cccc2c1 ; c1ccc2c(-c3ccc(N(c4ccc(-c5ccc6oc7ccccc7c6c5)cc4)c4cccc5c4oc4c(C6CCCCC6)cccc45)cc3)cccc2c1 ; c1ccc2c(-c3ccc(N(c4ccc(-c5cccc6oc7ccccc7c56)cc4)c4cccc5c4oc4c(C6CCCCC6)cccc45)cc3)cccc2c1]

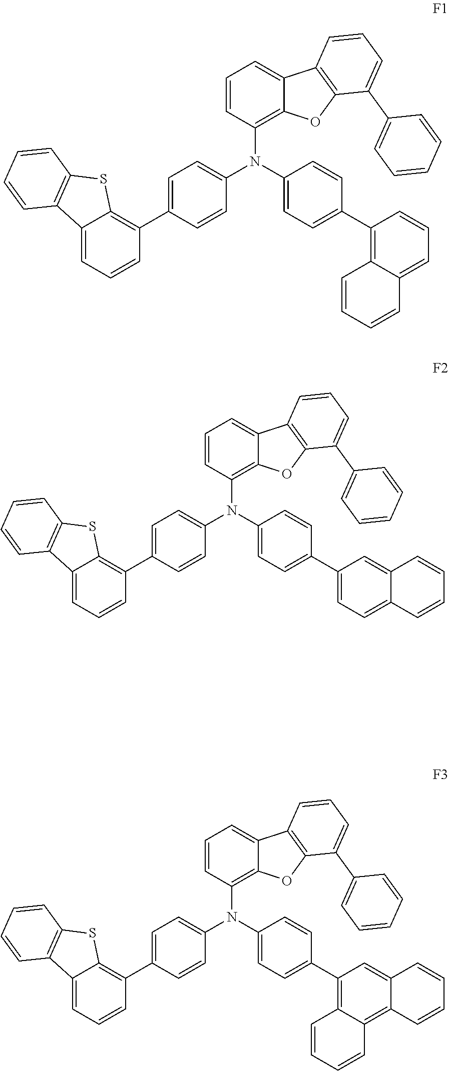 [SMILES: c1ccc(-c2cccc3c2oc2c(N(c4ccc(-c5cccc6ccccc56)cc4)c4ccc(-c5cccc6c5sc5ccccc56)cc4)cccc23)cc1 ; c1ccc(-c2cccc3c2oc2c(N(c4ccc(-c5ccc6ccccc6c5)cc4)c4ccc(-c5cccc6c5sc5ccccc56)cc4)cccc23)cc1 ; c1ccc(-c2cccc3c2oc2c(N(c4ccc(-c5cc6ccccc6c6ccccc56)cc4)c4ccc(-c5cccc6c5sc5ccccc56)cc4)cccc23)cc1]

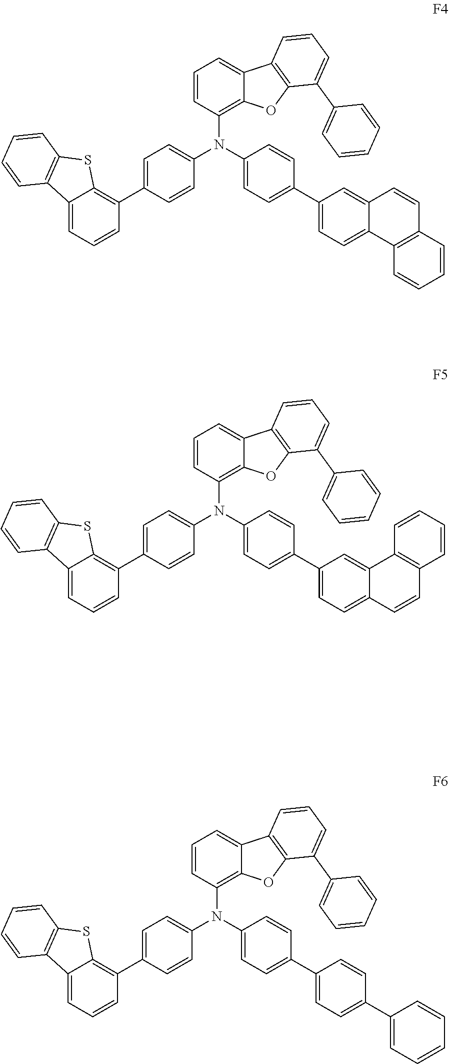

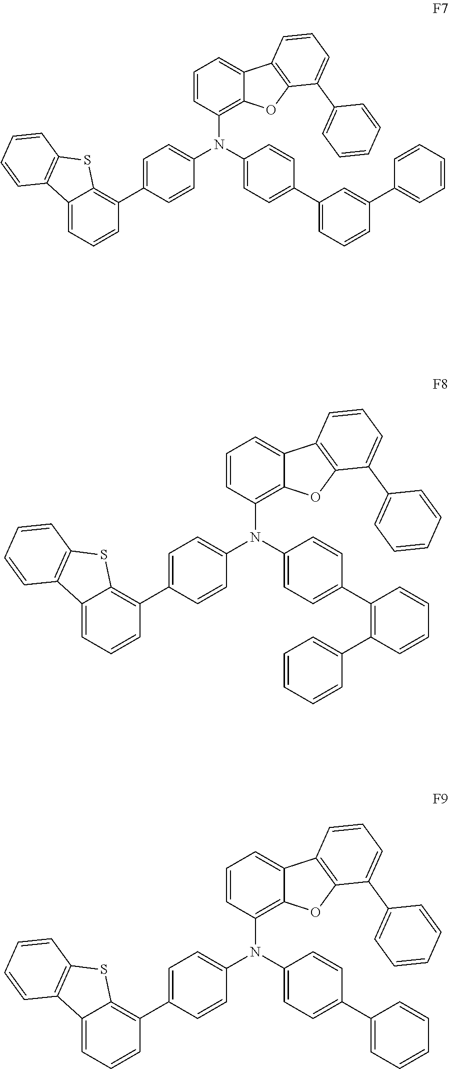 [SMILES: c1ccc(-c2cccc(-c3ccc(N(c4ccc(-c5cccc6c5sc5ccccc56)cc4)c4cccc5c4oc4c(-c6ccccc6)cccc45)cc3)c2)cc1 ; c1ccc(-c2ccccc2-c2ccc(N(c3ccc(-c4cccc5c4sc4ccccc45)cc3)c3cccc4c3oc3c(-c5ccccc5)cccc34)cc2)cc1 ; c1ccc(-c2ccc(N(c3ccc(-c4cccc5c4sc4ccccc45)cc3)c3cccc4c3oc3c(-c5ccccc5)cccc34)cc2)cc1]

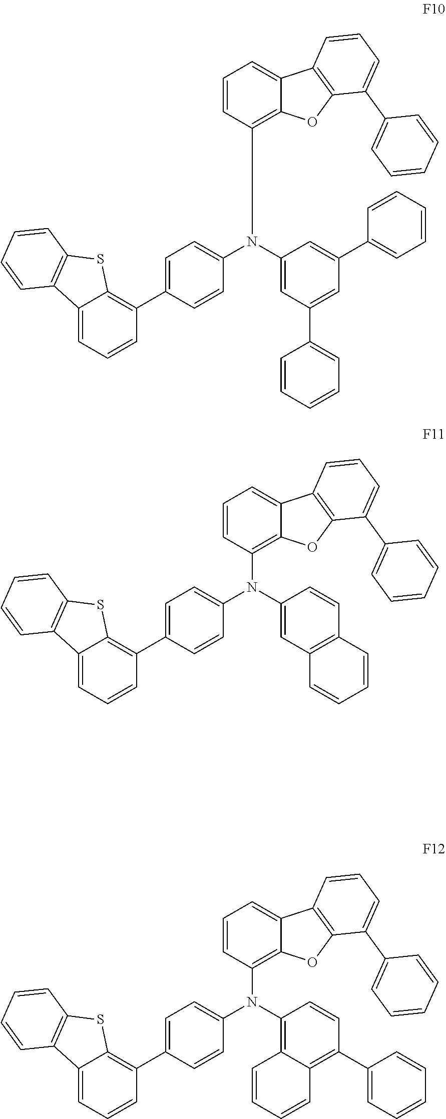 [SMILES: c1ccc(-c2cc(-c3ccccc3)cc(N(c3ccc(-c4cccc5c4sc4ccccc45)cc3)c3cccc4c3oc3c(-c5ccccc5)cccc34)c2)cc1 ; c1ccc(-c2cccc3c2oc2c(N(c4ccc(-c5cccc6c5sc5ccccc56)cc4)c4ccc5ccccc5c4)cccc23)cc1 ; c1ccc(-c2ccc(N(c3ccc(-c4cccc5c4sc4ccccc45)cc3)c3cccc4c3oc3c(-c5ccccc5)cccc34)c3ccccc23)cc1]

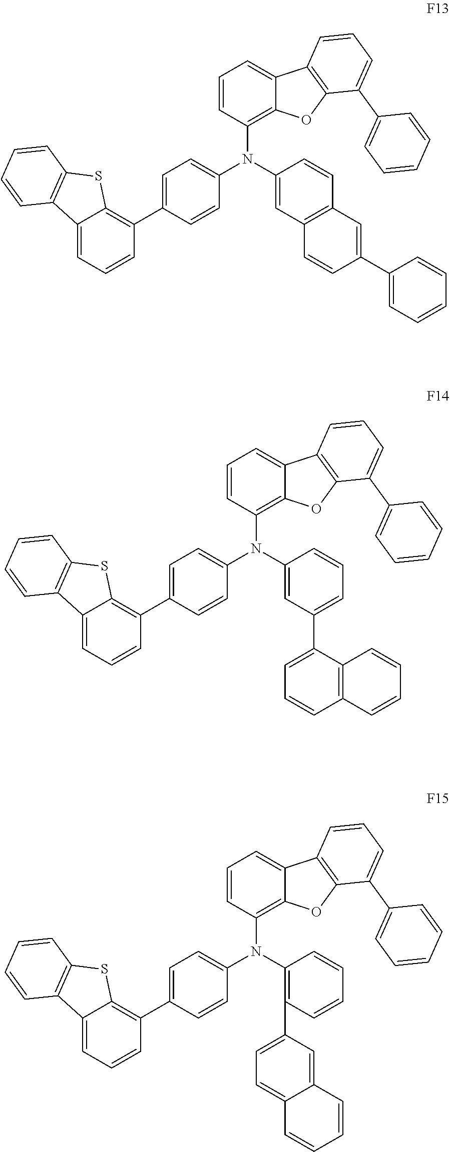 [SMILES: c1ccc(-c2ccc3cc(N(c4ccc(-c5cccc6c5sc5ccccc56)cc4)c4cccc5c4oc4c(-c6ccccc6)cccc45)ccc3c2)cc1 ; c1ccc(-c2cccc3c2oc2c(N(c4ccc(-c5cccc6c5sc5ccccc56)cc4)c4cccc(-c5cccc6ccccc56)c4)cccc23)cc1 ; c1ccc(-c2cccc3c2oc2c(N(c4ccc(-c5cccc6c5sc5ccccc56)cc4)c4ccccc4-c4ccc5ccccc5c4)cccc23)cc1]

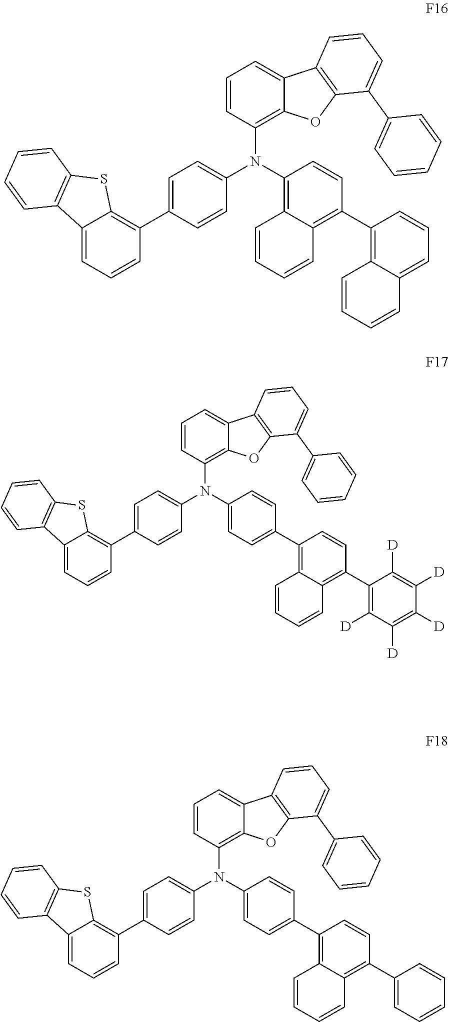 [SMILES: c1ccc(-c2cccc3c2oc2c(N(c4ccc(-c5cccc6c5sc5ccccc56)cc4)c4ccc(-c5cccc6ccccc56)c5ccccc45)cccc23)cc1 ; [2H]c1c([2H])c([2H])c(-c2ccc(-c3ccc(N(c4ccc(-c5cccc6c5sc5ccccc56)cc4)c4cccc5c4oc4c(-c6ccccc6)cccc45)cc3)c3ccccc23)c([2H])c1[2H] ; c1ccc(-c2ccc(-c3ccc(N(c4ccc(-c5cccc6c5sc5ccccc56)cc4)c4cccc5c4oc4c(-c6ccccc6)cccc45)cc3)c3ccccc23)cc1]

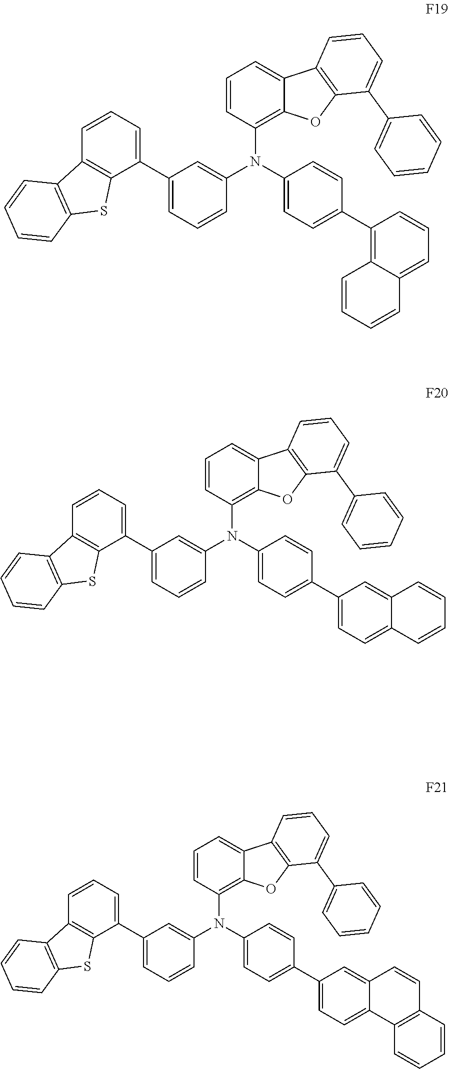 [SMILES: c1ccc(-c2cccc3c2oc2c(N(c4ccc(-c5cccc6ccccc56)cc4)c4cccc(-c5cccc6c5sc5ccccc56)c4)cccc23)cc1 ; c1ccc(-c2cccc3c2oc2c(N(c4ccc(-c5ccc6ccccc6c5)cc4)c4cccc(-c5cccc6c5sc5ccccc56)c4)cccc23)cc1 ; c1ccc(-c2cccc3c2oc2c(N(c4ccc(-c5ccc6c(ccc7ccccc76)c5)cc4)c4cccc(-c5cccc6c5sc5ccccc56)c4)cccc23)cc1]

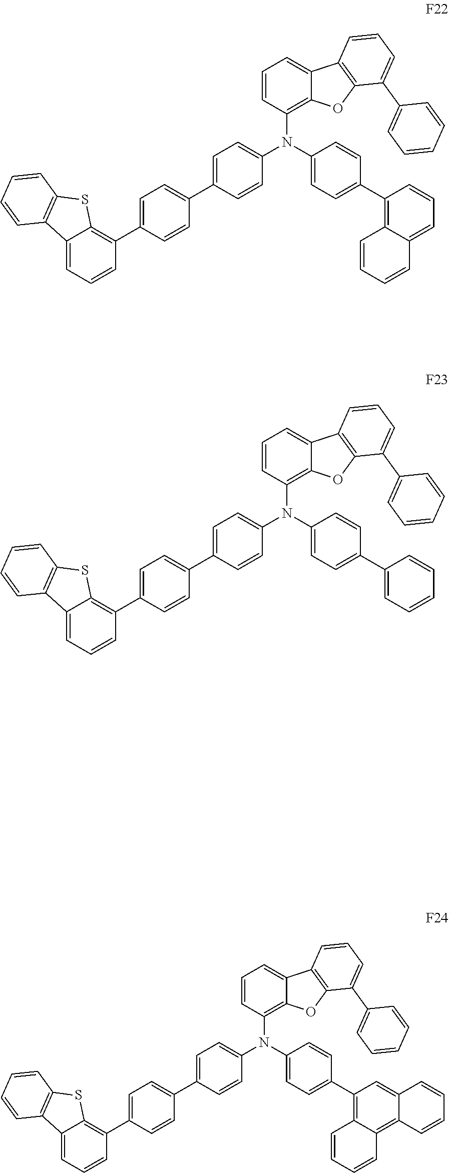 [SMILES: c1ccc(-c2cccc3c2oc2c(N(c4ccc(-c5ccc(-c6cccc7c6sc6ccccc67)cc5)cc4)c4ccc(-c5cccc6ccccc56)cc4)cccc23)cc1 ; c1ccc(-c2ccc(N(c3ccc(-c4ccc(-c5cccc6c5sc5ccccc56)cc4)cc3)c3cccc4c3oc3c(-c5ccccc5)cccc34)cc2)cc1 ; c1ccc(-c2cccc3c2oc2c(N(c4ccc(-c5ccc(-c6cccc7c6sc6ccccc67)cc5)cc4)c4ccc(-c5cc6ccccc6c6ccccc56)cc4)cccc23)cc1]

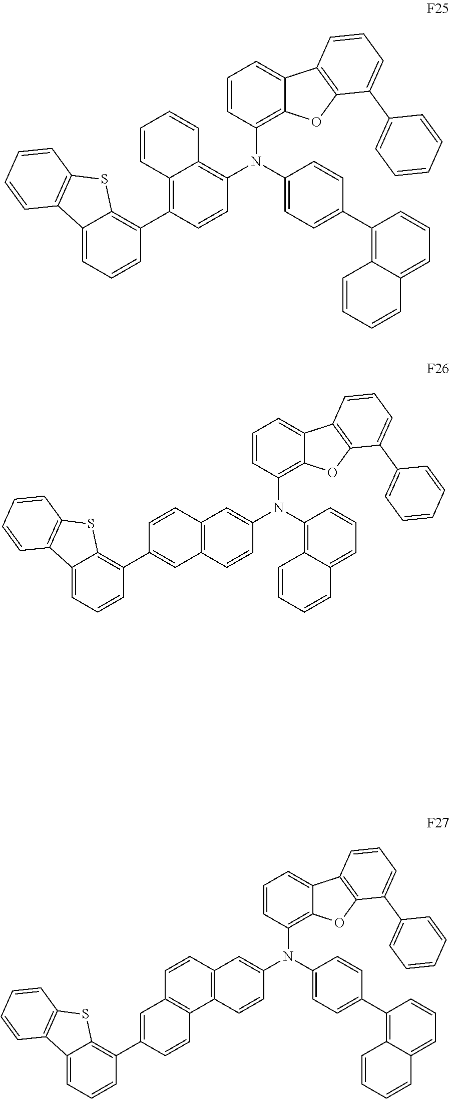 [SMILES: c1ccc(-c2cccc3c2oc2c(N(c4ccc(-c5cccc6ccccc56)cc4)c4ccc(-c5cccc6c5sc5ccccc56)c5ccccc45)cccc23)cc1 ; c1ccc(-c2cccc3c2oc2c(N(c4ccc5cc(-c6cccc7c6sc6ccccc67)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7cccc8c7sc7ccccc78)ccc65)c4)cccc23)cc1]

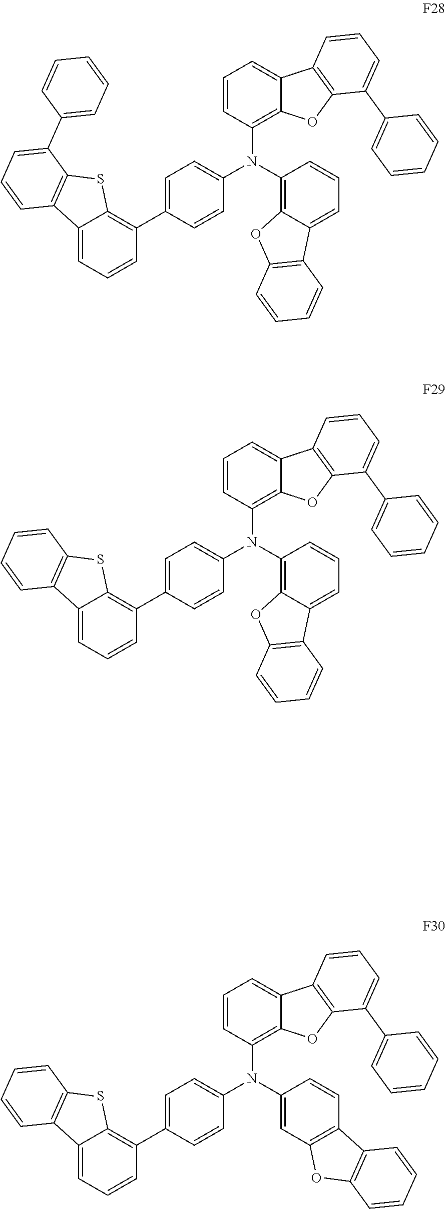 [SMILES: c1ccc(-c2cccc3c2oc2c(N(c4ccc(-c5cccc6c5sc5c(-c7ccccc7)cccc56)cc4)c4cccc5c4oc4ccccc45)cccc23)cc1 ; c1ccc(-c2cccc3c2oc2c(N(c4ccc(-c5cccc6c5sc5ccccc56)cc4)c4cccc5c4oc4ccccc45)cccc23)cc1 ; c1ccc(-c2cccc3c2oc2c(N(c4ccc(-c5cccc6c5sc5ccccc56)cc4)c4ccc5c(c4)oc4ccccc45)cccc23)cc1]

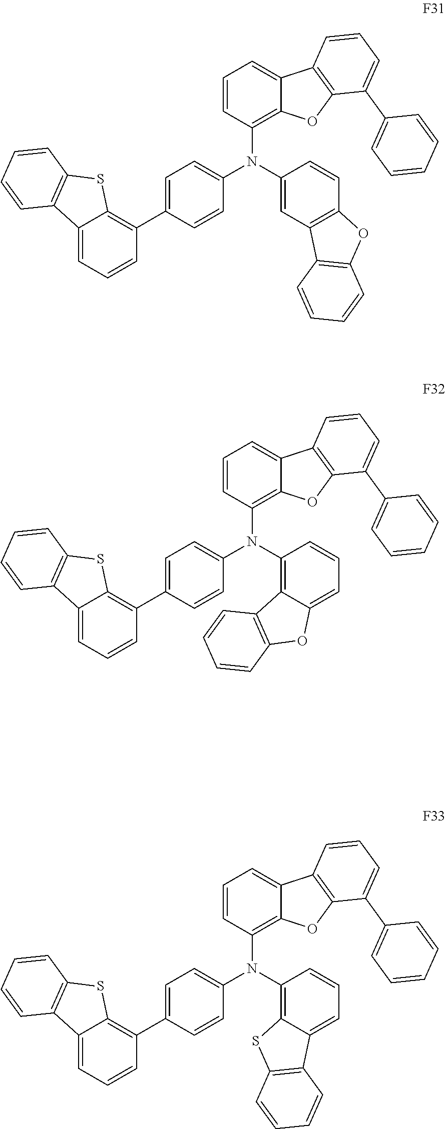 [SMILES: c1ccc(-c2cccc3c2oc2c(N(c4ccc(-c5cccc6c5sc5ccccc56)cc4)c4ccc5oc6ccccc6c5c4)cccc23)cc1 ; c1ccc(-c2cccc3c2oc2c(N(c4ccc(-c5cccc6c5sc5ccccc56)cc4)c4cccc5oc6ccccc6c45)cccc23)cc1 ; c1ccc(-c2cccc3c2oc2c(N(c4ccc(-c5cccc6c5sc5ccccc56)cc4)c4cccc5c4sc4ccccc45)cccc23)cc1]

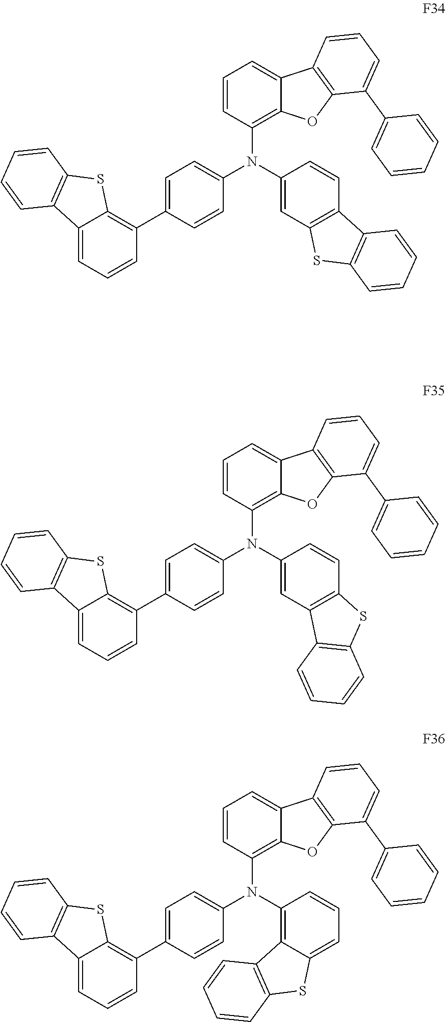 [SMILES: c1ccc(-c2cccc3c2oc2c(N(c4ccc(-c5cccc6c5sc5ccccc56)cc4)c4ccc5c(c4)sc4ccccc45)cccc23)cc1 ; c1ccc(-c2cccc3c2oc2c(N(c4ccc(-c5cccc6c5sc5ccccc56)cc4)c4ccc5sc6ccccc6c5c4)cccc23)cc1 ; c1ccc(-c2cccc3c2oc2c(N(c4ccc(-c5cccc6c5sc5ccccc56)cc4)c4cccc5sc6ccccc6c45)cccc23)cc1]

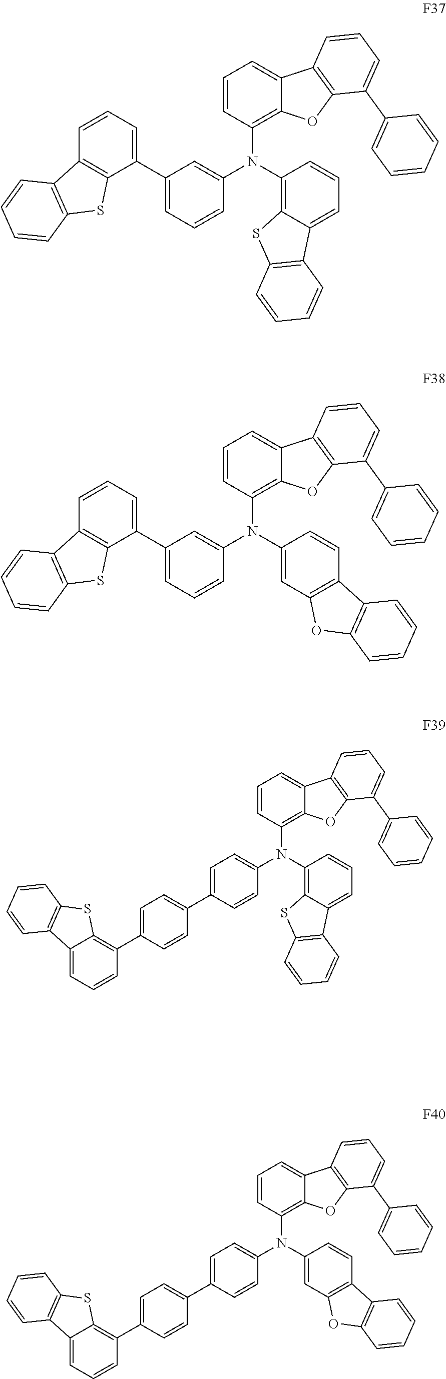 [SMILES: c1ccc(-c2cccc3c2oc2c(N(c4cccc(-c5cccc6c5sc5ccccc56)c4)c4cccc5c4sc4ccccc45)cccc23)cc1 ; c1ccc(-c2cccc3c2oc2c(N(c4cccc(-c5cccc6c5sc5ccccc56)c4)c4ccc5c(c4)oc4ccccc45)cccc23)cc1 ; c1ccc(-c2cccc3c2oc2c(N(c4ccc(-c5ccc(-c6cccc7c6sc6ccccc67)cc5)cc4)c4cccc5c4sc4ccccc45)cccc23)cc1 ; c1ccc(-c2cccc3c2oc2c(N(c4ccc(-c5ccc(-c6cccc7c6sc6ccccc67)cc5)cc4)c4ccc5c(c4)oc4ccccc45)cccc23)cc1]

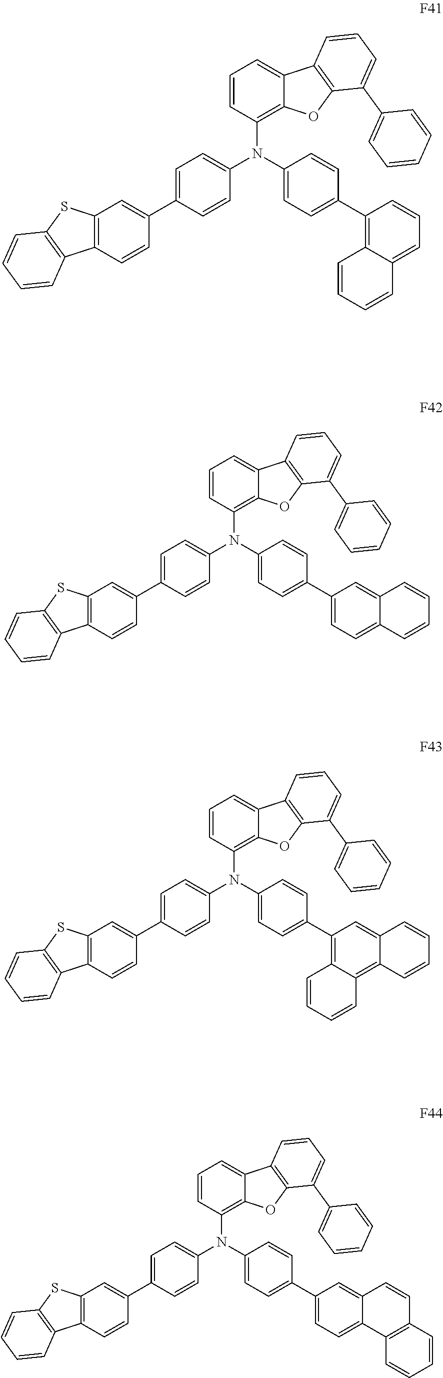 [SMILES: c1ccc(-c2cccc3c2oc2c(N(c4ccc(-c5ccc6c(c5)sc5ccccc56)cc4)c4ccc(-c5cccc6ccccc56)cc4)cccc23)cc1 ; c1ccc(-c2cccc3c2oc2c(N(c4ccc(-c5ccc6ccccc6c5)cc4)c4ccc(-c5ccc6c(c5)sc5ccccc56)cc4)cccc23)cc1 ; c1ccc(-c2cccc3c2oc2c(N(c4ccc(-c5ccc6c(c5)sc5ccccc56)cc4)c4ccc(-c5cc6ccccc6c6ccccc56)cc4)cccc23)cc1 ; c1ccc(-c2cccc3c2oc2c(N(c4ccc(-c5ccc6c(ccc7ccccc76)c5)cc4)c4ccc(-c5ccc6c(c5)sc5ccccc56)cc4)cccc23)cc1]

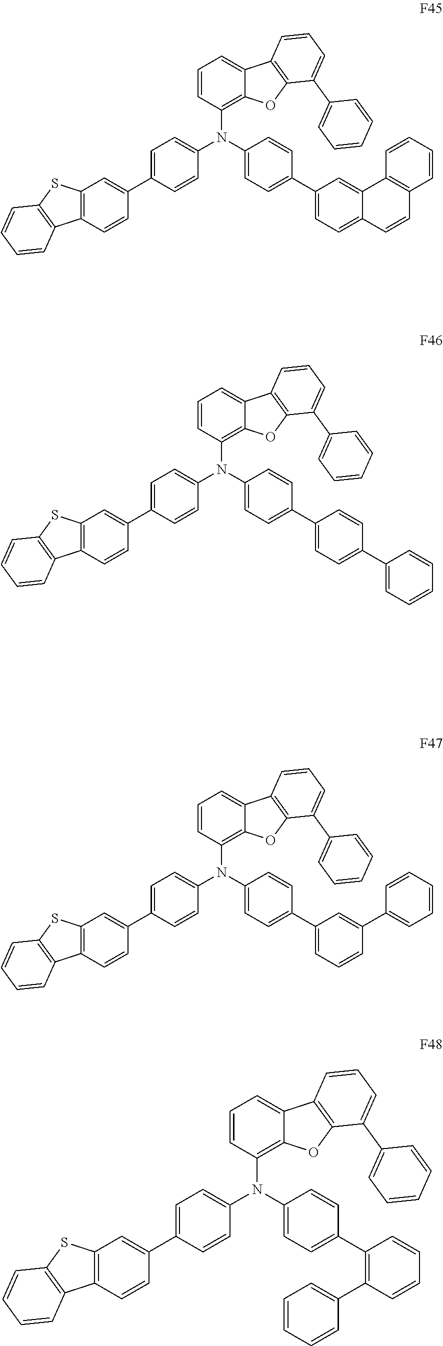 [SMILES: c1ccc(-c2cccc3c2oc2c(N(c4ccc(-c5ccc6c(c5)sc5ccccc56)cc4)c4ccc(-c5ccc6ccc7ccccc7c6c5)cc4)cccc23)cc1 ; c1ccc(-c2ccc(-c3ccc(N(c4ccc(-c5ccc6c(c5)sc5ccccc56)cc4)c4cccc5c4oc4c(-c6ccccc6)cccc45)cc3)cc2)cc1 ; c1ccc(-c2cccc(-c3ccc(N(c4ccc(-c5ccc6c(c5)sc5ccccc56)cc4)c4cccc5c4oc4c(-c6ccccc6)cccc45)cc3)c2)cc1 ; c1ccc(-c2ccccc2-c2ccc(N(c3ccc(-c4ccc5c(c4)sc4ccccc45)cc3)c3cccc4c3oc3c(-c5ccccc5)cccc34)cc2)cc1]

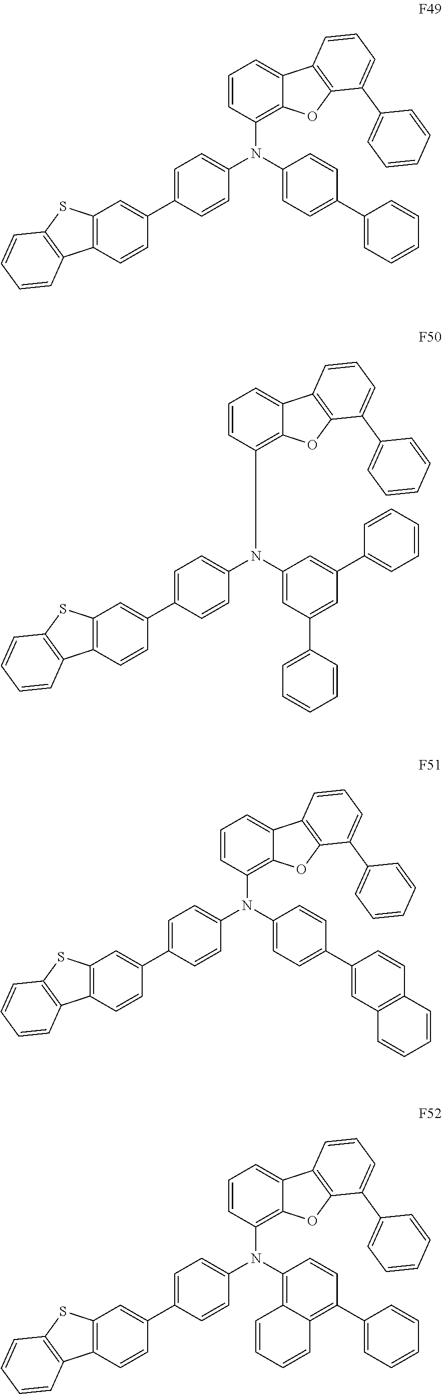 [SMILES: c1ccc(-c2ccc(N(c3ccc(-c4ccc5c(c4)sc4ccccc45)cc3)c3cccc4c3oc3c(-c5ccccc5)cccc34)cc2)cc1 ; c1ccc(-c2cc(-c3ccccc3)cc(N(c3ccc(-c4ccc5c(c4)sc4ccccc45)cc3)c3cccc4c3oc3c(-c5ccccc5)cccc34)c2)cc1 ; c1ccc(-c2cccc3c2oc2c(N(c4ccc(-c5ccc6ccccc6c5)cc4)c4ccc(-c5ccc6c(c5)sc5ccccc56)cc4)cccc23)cc1 ; c1ccc(-c2ccc(N(c3ccc(-c4ccc5c(c4)sc4ccccc45)cc3)c3cccc4c3oc3c(-c5ccccc5)cccc34)c3ccccc23)cc1]

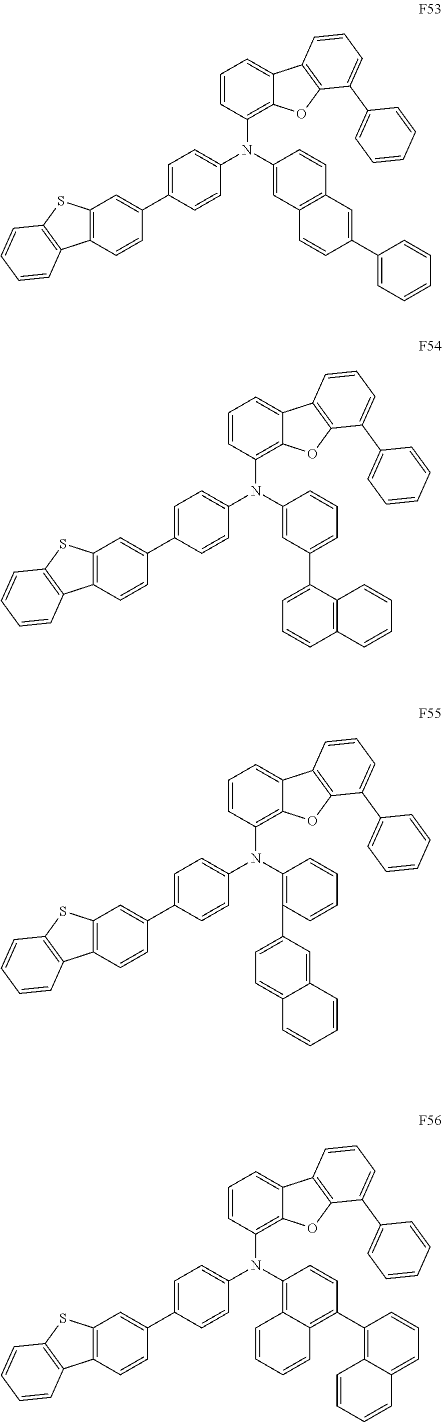 [SMILES: c1ccc(-c2ccc3cc(N(c4ccc(-c5ccc6c(c5)sc5ccccc56)cc4)c4cccc5c4oc4c(-c6ccccc6)cccc45)ccc3c2)cc1 ; c1ccc(-c2cccc3c2oc2c(N(c4ccc(-c5ccc6c(c5)sc5ccccc56)cc4)c4cccc(-c5cccc6ccccc56)c4)cccc23)cc1 ; c1ccc(-c2cccc3c2oc2c(N(c4ccc(-c5ccc6c(c5)sc5ccccc56)cc4)c4ccccc4-c4ccc5ccccc5c4)cccc23)cc1 ; c1ccc(-c2cccc3c2oc2c(N(c4ccc(-c5ccc6c(c5)sc5ccccc56)cc4)c4ccc(-c5cccc6ccccc56)c5ccccc45)cccc23)cc1]

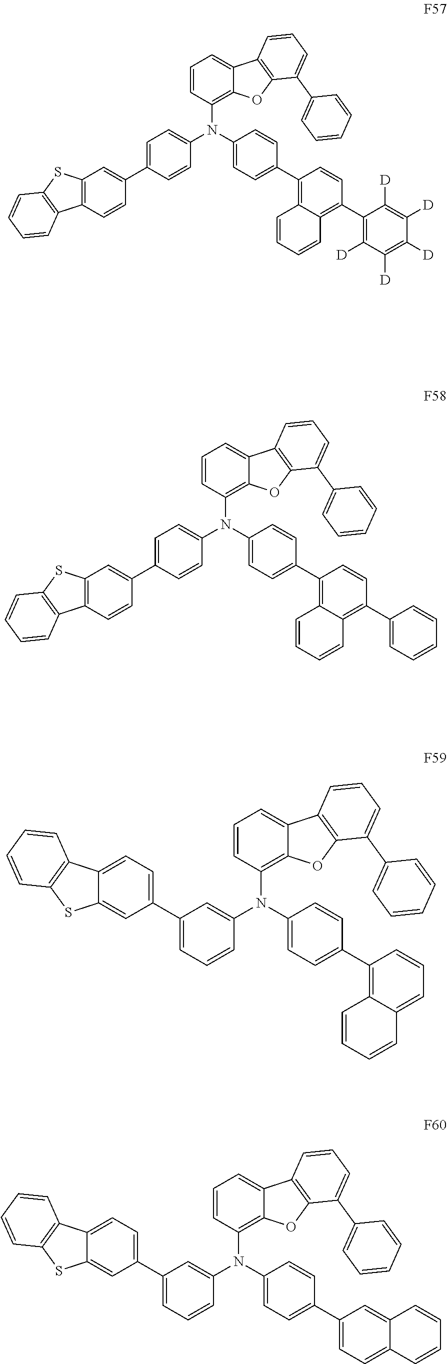 [SMILES: [2H]c1c([2H])c([2H])c(-c2ccc(-c3ccc(N(c4ccc(-c5ccc6c(c5)sc5ccccc56)cc4)c4cccc5c4oc4c(-c6ccccc6)cccc45)cc3)c3ccccc23)c([2H])c1[2H] ; c1ccc(-c2ccc(-c3ccc(N(c4ccc(-c5ccc6c(c5)sc5ccccc56)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5ccc6c(c5)sc5ccccc56)c4)cccc23)cc1 ; c1ccc(-c2cccc3c2oc2c(N(c4ccc(-c5ccc6ccccc6c5)cc4)c4cccc(-c5ccc6c(c5)sc5ccccc56)c4)cccc23)cc1]

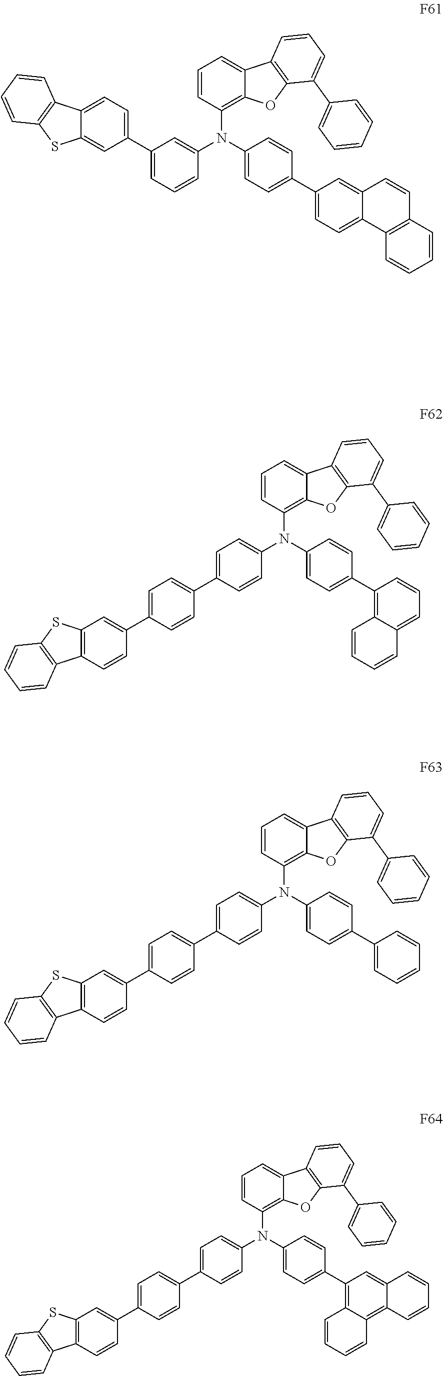 [SMILES: c1ccc(-c2cccc3c2oc2c(N(c4ccc(-c5ccc6c(ccc7ccccc76)c5)cc4)c4cccc(-c5ccc6c(c5)sc5ccccc56)c4)cccc23)cc1 ; c1ccc(-c2cccc3c2oc2c(N(c4ccc(-c5ccc(-c6ccc7c(c6)sc6ccccc67)cc5)cc4)c4ccc(-c5cccc6ccccc56)cc4)cccc23)cc1 ; c1ccc(-c2ccc(N(c3ccc(-c4ccc(-c5ccc6c(c5)sc5ccccc56)cc4)cc3)c3cccc4c3oc3c(-c5ccccc5)cccc34)cc2)cc1 ; c1ccc(-c2cccc3c2oc2c(N(c4ccc(-c5ccc(-c6ccc7c(c6)sc6ccccc67)cc5)cc4)c4ccc(-c5cc6ccccc6c6ccccc56)cc4)cccc23)cc1]

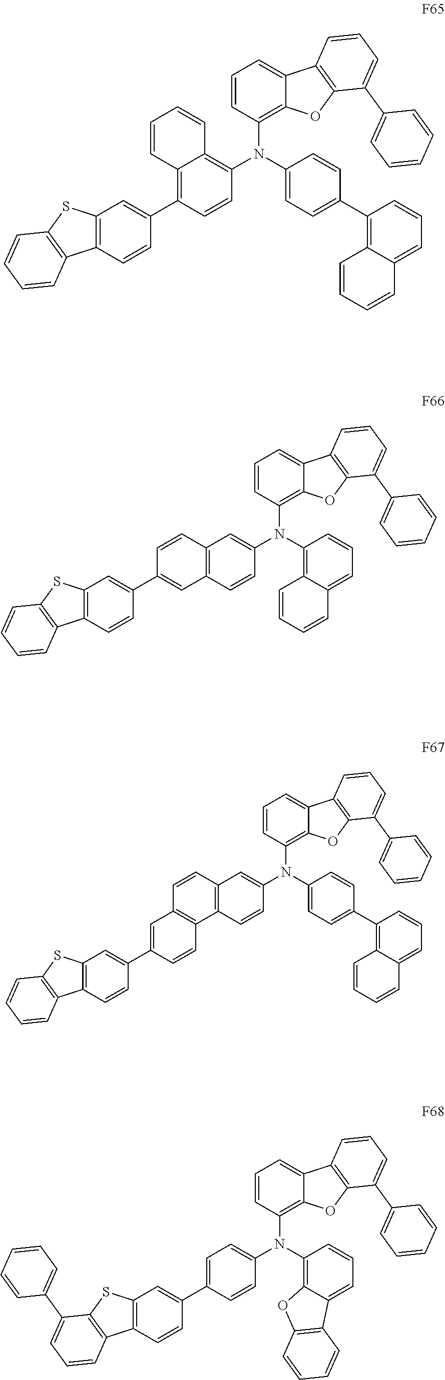 [SMILES: c1ccc(-c2cccc3c2oc2c(N(c4ccc(-c5cccc6ccccc56)cc4)c4ccc(-c5ccc6c(c5)sc5ccccc56)c5ccccc45)cccc23)cc1 ; c1ccc(-c2cccc3c2oc2c(N(c4ccc5cc(-c6ccc7c(c6)sc6ccccc67)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7ccc8c(c7)sc7ccccc78)ccc65)c4)cccc23)cc1 ; c1ccc(-c2cccc3c2oc2c(N(c4ccc(-c5ccc6c(c5)sc5c(-c7ccccc7)cccc56)cc4)c4cccc5c4oc4ccccc45)cccc23)cc1]

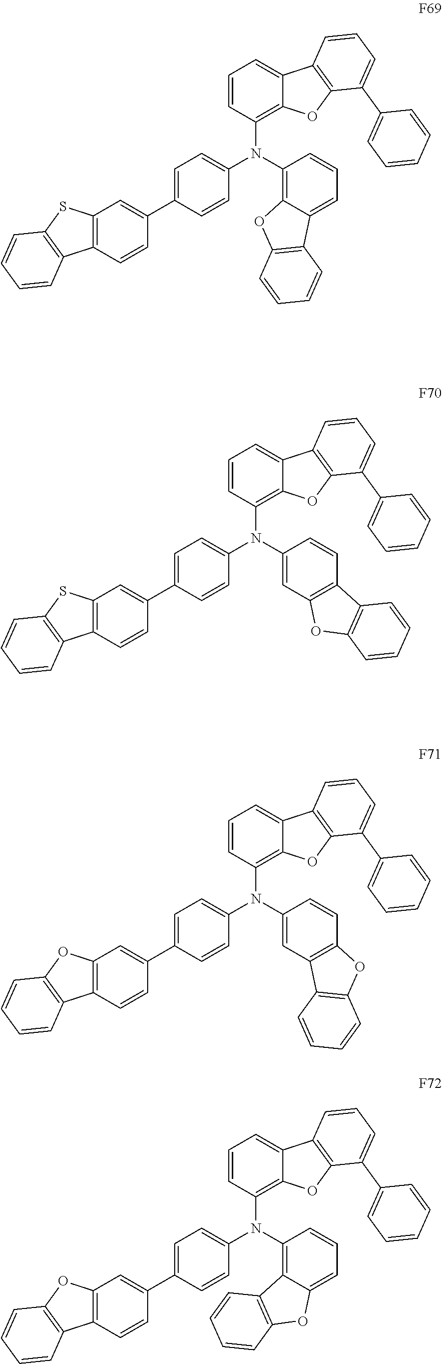 [SMILES: c1ccc(-c2cccc3c2oc2c(N(c4ccc(-c5ccc6c(c5)sc5ccccc56)cc4)c4cccc5c4oc4ccccc45)cccc23)cc1 ; c1ccc(-c2cccc3c2oc2c(N(c4ccc(-c5ccc6c(c5)sc5ccccc56)cc4)c4ccc5c(c4)oc4ccccc45)cccc23)cc1 ; c1ccc(-c2cccc3c2oc2c(N(c4ccc(-c5ccc6c(c5)oc5ccccc56)cc4)c4ccc5oc6ccccc6c5c4)cccc23)cc1 ; c1ccc(-c2cccc3c2oc2c(N(c4ccc(-c5ccc6c(c5)oc5ccccc56)cc4)c4cccc5oc6ccccc6c45)cccc23)cc1]

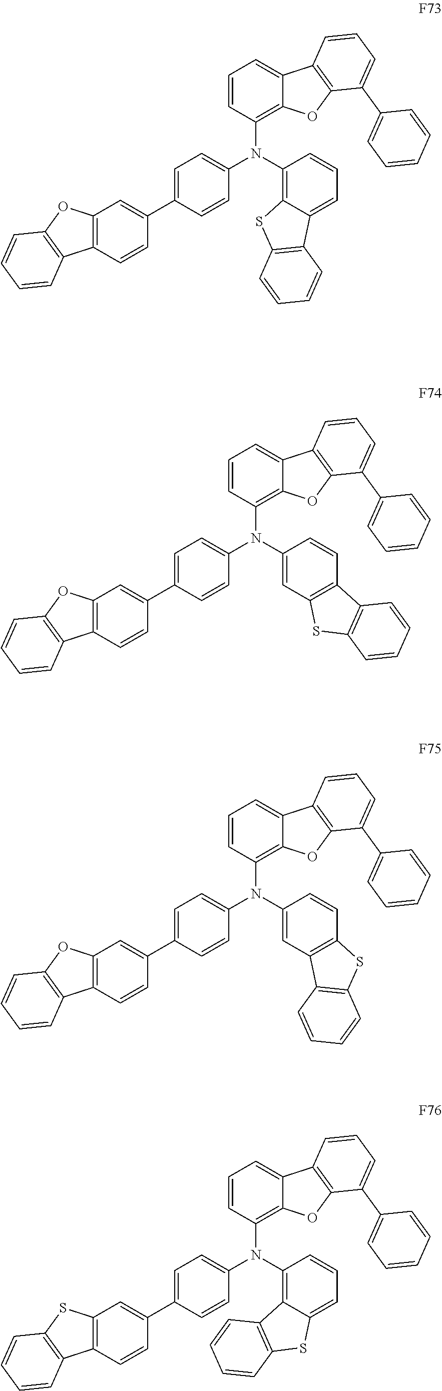 [SMILES: c1ccc(-c2cccc3c2oc2c(N(c4ccc(-c5ccc6c(c5)oc5ccccc56)cc4)c4cccc5c4sc4ccccc45)cccc23)cc1 ; c1ccc(-c2cccc3c2oc2c(N(c4ccc(-c5ccc6c(c5)oc5ccccc56)cc4)c4ccc5c(c4)sc4ccccc45)cccc23)cc1 ; c1ccc(-c2cccc3c2oc2c(N(c4ccc(-c5ccc6c(c5)oc5ccccc56)cc4)c4ccc5sc6ccccc6c5c4)cccc23)cc1 ; c1ccc(-c2cccc3c2oc2c(N(c4ccc(-c5ccc6c(c5)sc5ccccc56)cc4)c4cccc5sc6ccccc6c45)cccc23)cc1]

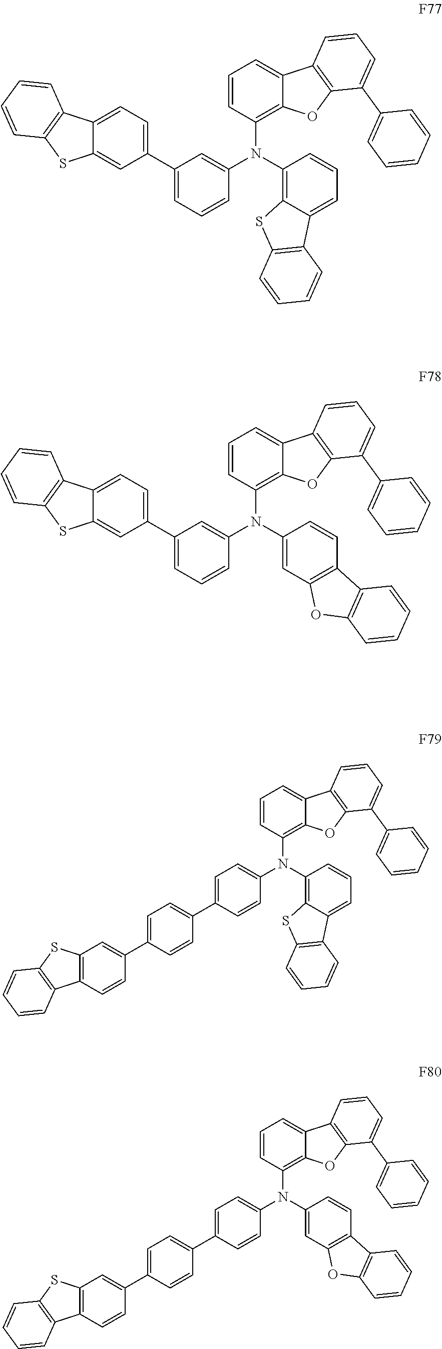 [SMILES: c1ccc(-c2cccc3c2oc2c(N(c4cccc(-c5ccc6c(c5)sc5ccccc56)c4)c4cccc5c4sc4ccccc45)cccc23)cc1 ; c1ccc(-c2cccc3c2oc2c(N(c4cccc(-c5ccc6c(c5)sc5ccccc56)c4)c4ccc5c(c4)oc4ccccc45)cccc23)cc1 ; c1ccc(-c2cccc3c2oc2c(N(c4ccc(-c5ccc(-c6ccc7c(c6)sc6ccccc67)cc5)cc4)c4cccc5c4sc4ccccc45)cccc23)cc1 ; c1ccc(-c2cccc3c2oc2c(N(c4ccc(-c5ccc(-c6ccc7c(c6)sc6ccccc67)cc5)cc4)c4ccc5c(c4)oc4ccccc45)cccc23)cc1]

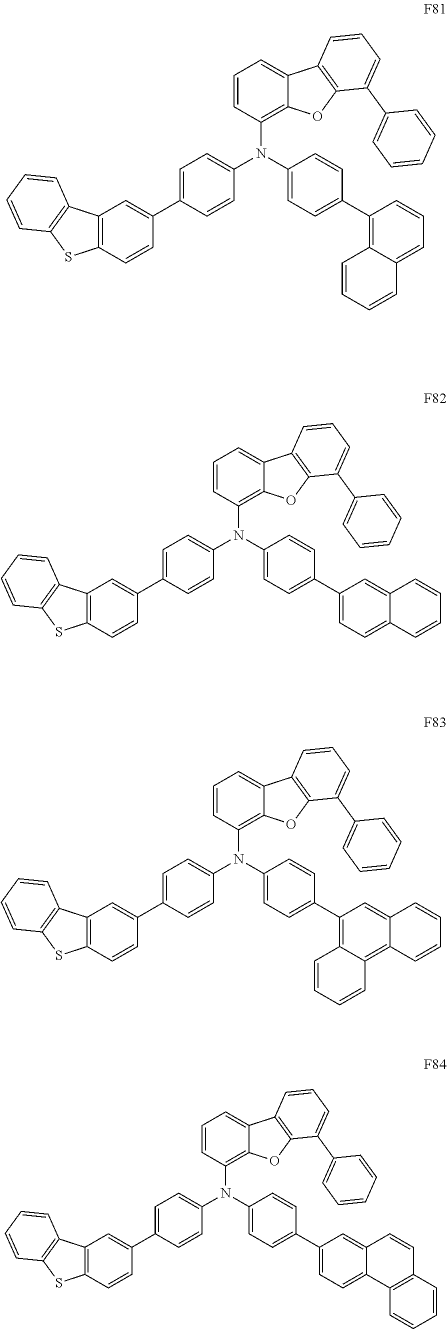 [SMILES: c1ccc(-c2cccc3c2oc2c(N(c4ccc(-c5ccc6sc7ccccc7c6c5)cc4)c4ccc(-c5cccc6ccccc56)cc4)cccc23)cc1 ; c1ccc(-c2cccc3c2oc2c(N(c4ccc(-c5ccc6ccccc6c5)cc4)c4ccc(-c5ccc6sc7ccccc7c6c5)cc4)cccc23)cc1 ; c1ccc(-c2cccc3c2oc2c(N(c4ccc(-c5ccc6sc7ccccc7c6c5)cc4)c4ccc(-c5cc6ccccc6c6ccccc56)cc4)cccc23)cc1 ; c1ccc(-c2cccc3c2oc2c(N(c4ccc(-c5ccc6c(ccc7ccccc76)c5)cc4)c4ccc(-c5ccc6sc7ccccc7c6c5)cc4)cccc23)cc1]

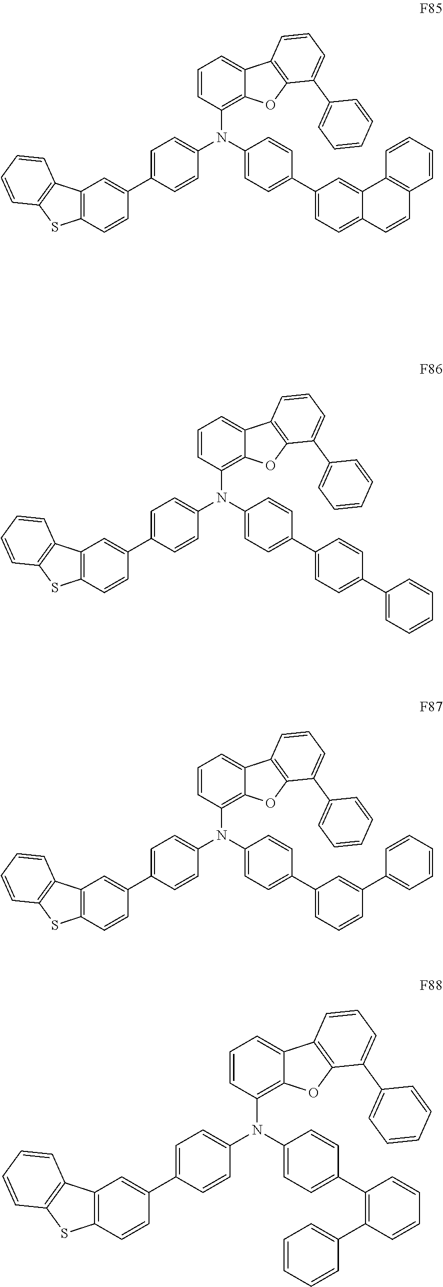 [SMILES: c1ccc(-c2cccc3c2oc2c(N(c4ccc(-c5ccc6ccc7ccccc7c6c5)cc4)c4ccc(-c5ccc6sc7ccccc7c6c5)cc4)cccc23)cc1 ; c1ccc(-c2ccc(-c3ccc(N(c4ccc(-c5ccc6sc7ccccc7c6c5)cc4)c4cccc5c4oc4c(-c6ccccc6)cccc45)cc3)cc2)cc1 ; c1ccc(-c2cccc(-c3ccc(N(c4ccc(-c5ccc6sc7ccccc7c6c5)cc4)c4cccc5c4oc4c(-c6ccccc6)cccc45)cc3)c2)cc1 ; c1ccc(-c2ccccc2-c2ccc(N(c3ccc(-c4ccc5sc6ccccc6c5c4)cc3)c3cccc4c3oc3c(-c5ccccc5)cccc34)cc2)cc1]

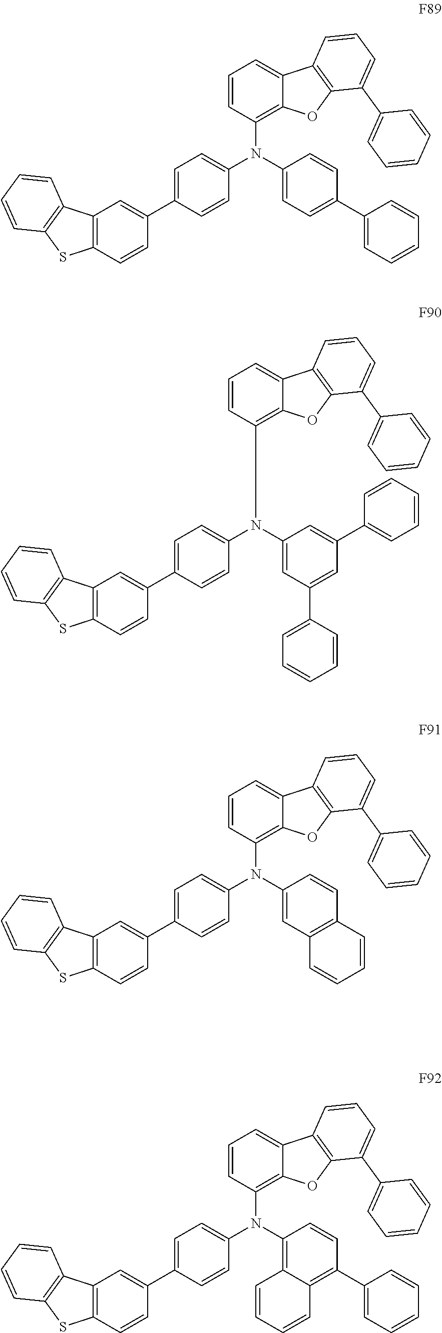 [SMILES: c1ccc(-c2ccc(N(c3ccc(-c4ccc5sc6ccccc6c5c4)cc3)c3cccc4c3oc3c(-c5ccccc5)cccc34)cc2)cc1 ; c1ccc(-c2cc(-c3ccccc3)cc(N(c3ccc(-c4ccc5sc6ccccc6c5c4)cc3)c3cccc4c3oc3c(-c5ccccc5)cccc34)c2)cc1 ; c1ccc(-c2cccc3c2oc2c(N(c4ccc(-c5ccc6sc7ccccc7c6c5)cc4)c4ccc5ccccc5c4)cccc23)cc1 ; c1ccc(-c2ccc(N(c3ccc(-c4ccc5sc6ccccc6c5c4)cc3)c3cccc4c3oc3c(-c5ccccc5)cccc34)c3ccccc23)cc1]

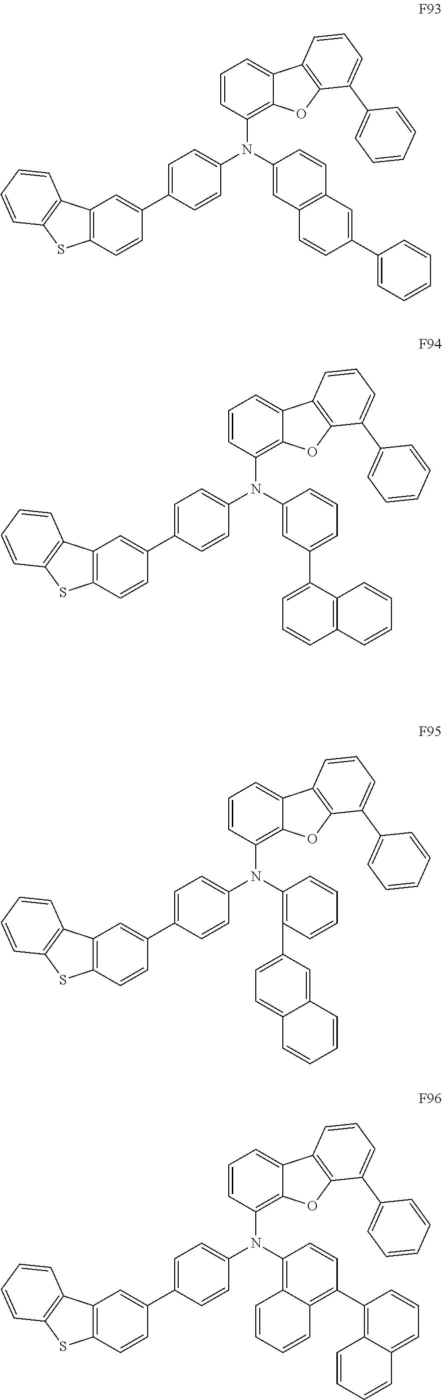 [SMILES: c1ccc(-c2ccc3cc(N(c4ccc(-c5ccc6sc7ccccc7c6c5)cc4)c4cccc5c4oc4c(-c6ccccc6)cccc45)ccc3c2)cc1 ; c1ccc(-c2cccc3c2oc2c(N(c4ccc(-c5ccc6sc7ccccc7c6c5)cc4)c4cccc(-c5cccc6ccccc56)c4)cccc23)cc1 ; c1ccc(-c2cccc3c2oc2c(N(c4ccc(-c5ccc6sc7ccccc7c6c5)cc4)c4ccccc4-c4ccc5ccccc5c4)cccc23)cc1 ; c1ccc(-c2cccc3c2oc2c(N(c4ccc(-c5ccc6sc7ccccc7c6c5)cc4)c4ccc(-c5cccc6ccccc56)c5ccccc45)cccc23)cc1]

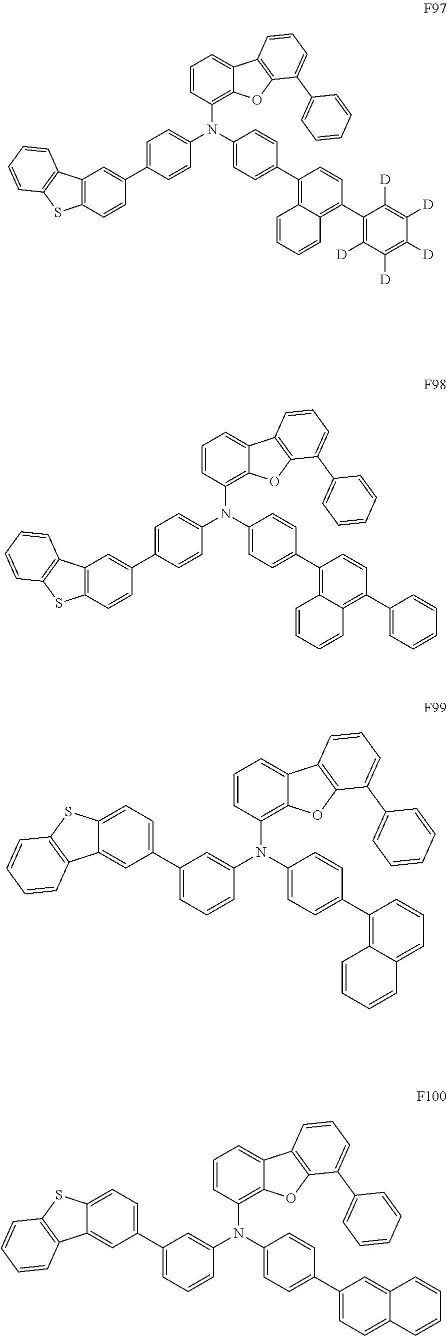 [SMILES: [2H]c1c([2H])c([2H])c(-c2ccc(-c3ccc(N(c4ccc(-c5ccc6sc7ccccc7c6c5)cc4)c4cccc5c4oc4c(-c6ccccc6)cccc45)cc3)c3ccccc23)c([2H])c1[2H] ; c1ccc(-c2ccc(-c3ccc(N(c4ccc(-c5ccc6sc7ccccc7c6c5)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5ccc6sc7ccccc7c6c5)c4)cccc23)cc1 ; c1ccc(-c2cccc3c2oc2c(N(c4ccc(-c5ccc6ccccc6c5)cc4)c4cccc(-c5ccc6sc7ccccc7c6c5)c4)cccc23)cc1]

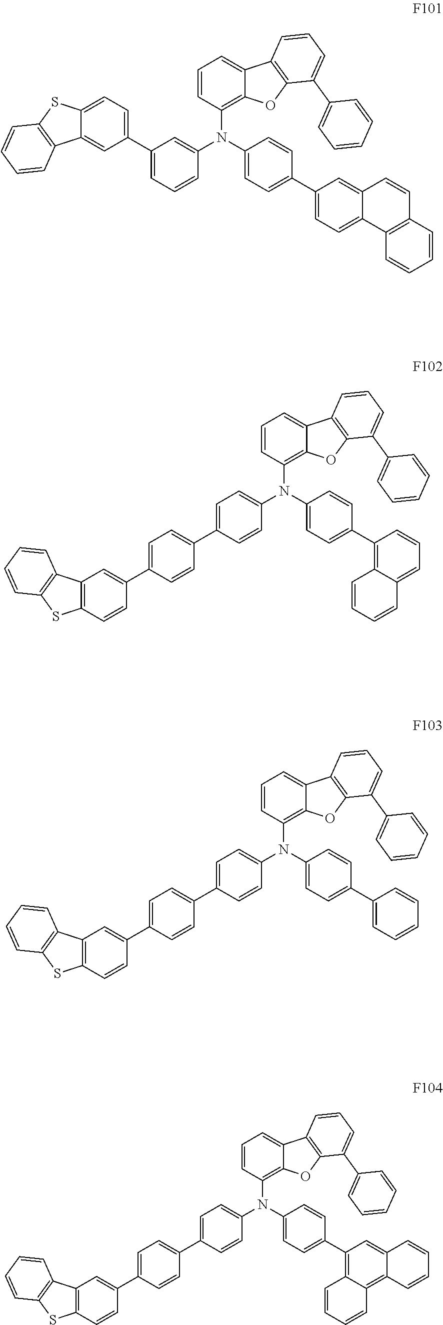 [SMILES: c1ccc(-c2cccc3c2oc2c(N(c4ccc(-c5ccc6c(ccc7ccccc76)c5)cc4)c4cccc(-c5ccc6sc7ccccc7c6c5)c4)cccc23)cc1 ; c1ccc(-c2cccc3c2oc2c(N(c4ccc(-c5ccc(-c6ccc7sc8ccccc8c7c6)cc5)cc4)c4ccc(-c5cccc6ccccc56)cc4)cccc23)cc1 ; c1ccc(-c2ccc(N(c3ccc(-c4ccc(-c5ccc6sc7ccccc7c6c5)cc4)cc3)c3cccc4c3oc3c(-c5ccccc5)cccc34)cc2)cc1 ; c1ccc(-c2cccc3c2oc2c(N(c4ccc(-c5ccc(-c6ccc7sc8ccccc8c7c6)cc5)cc4)c4ccc(-c5cc6ccccc6c6ccccc56)cc4)cccc23)cc1]

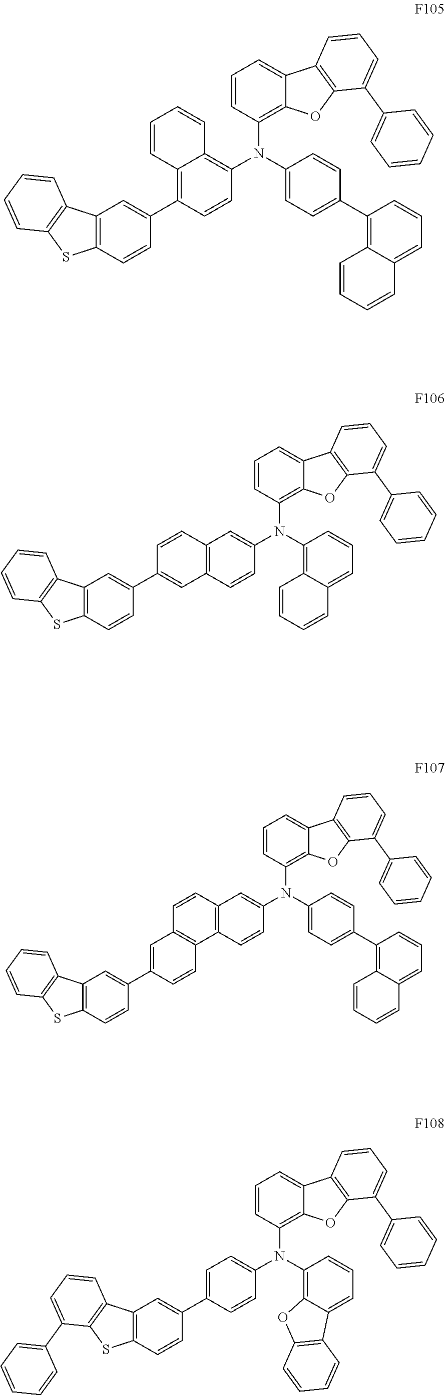 [SMILES: c1ccc(-c2cccc3c2oc2c(N(c4ccc(-c5cccc6ccccc56)cc4)c4ccc(-c5ccc6sc7ccccc7c6c5)c5ccccc45)cccc23)cc1 ; c1ccc(-c2cccc3c2oc2c(N(c4ccc5cc(-c6ccc7sc8ccccc8c7c6)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7ccc8sc9ccccc9c8c7)ccc65)c4)cccc23)cc1 ; c1ccc(-c2cccc3c2oc2c(N(c4ccc(-c5ccc6sc7c(-c8ccccc8)cccc7c6c5)cc4)c4cccc5c4oc4ccccc45)cccc23)cc1]

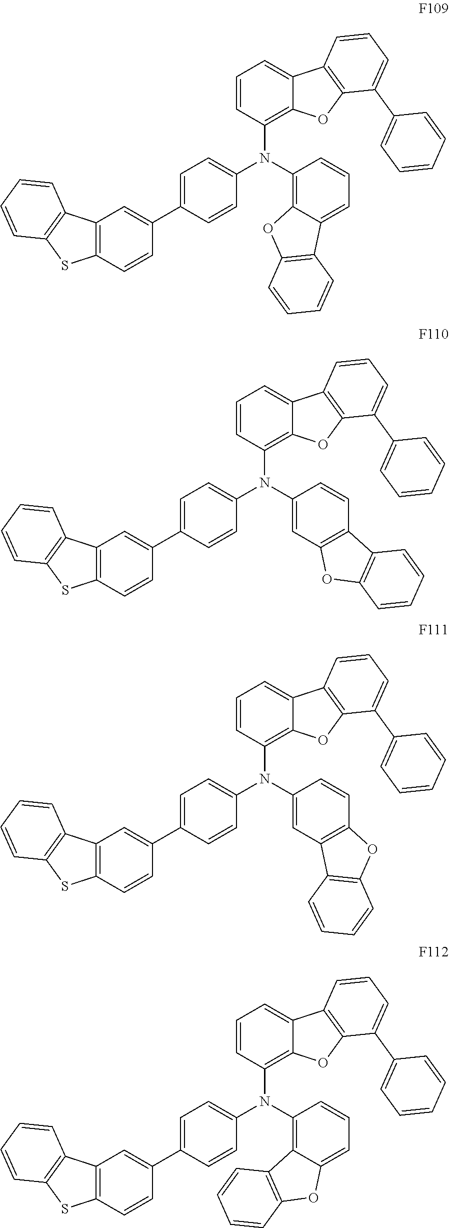

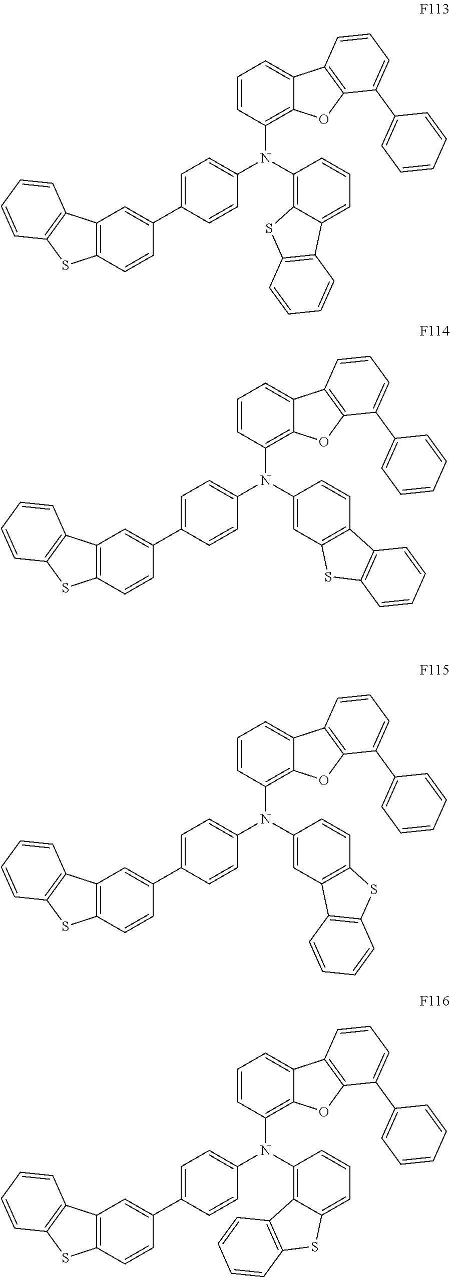 [SMILES: c1ccc(-c2cccc3c2oc2c(N(c4ccc(-c5ccc6sc7ccccc7c6c5)cc4)c4cccc5c4sc4ccccc45)cccc23)cc1 ; c1ccc(-c2cccc3c2oc2c(N(c4ccc(-c5ccc6sc7ccccc7c6c5)cc4)c4ccc5c(c4)sc4ccccc45)cccc23)cc1 ; c1ccc(-c2cccc3c2oc2c(N(c4ccc(-c5ccc6sc7ccccc7c6c5)cc4)c4ccc5sc6ccccc6c5c4)cccc23)cc1 ; c1ccc(-c2cccc3c2oc2c(N(c4ccc(-c5ccc6sc7ccccc7c6c5)cc4)c4cccc5sc6ccccc6c45)cccc23)cc1]

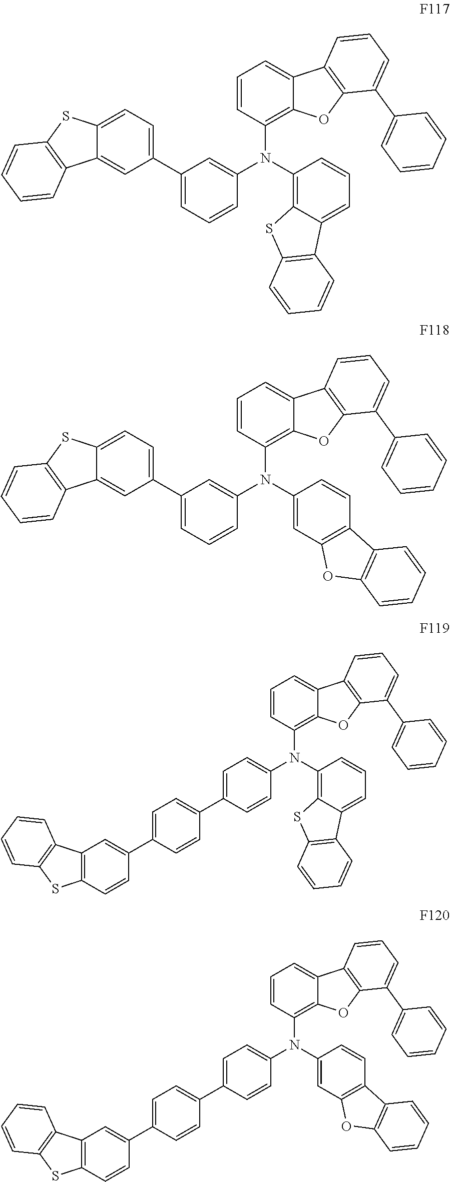 [SMILES: c1ccc(-c2cccc3c2oc2c(N(c4cccc(-c5ccc6sc7ccccc7c6c5)c4)c4cccc5c4sc4ccccc45)cccc23)cc1 ; c1ccc(-c2cccc3c2oc2c(N(c4cccc(-c5ccc6sc7ccccc7c6c5)c4)c4ccc5c(c4)oc4ccccc45)cccc23)cc1 ; c1ccc(-c2cccc3c2oc2c(N(c4ccc(-c5ccc(-c6ccc7sc8ccccc8c7c6)cc5)cc4)c4cccc5c4sc4ccccc45)cccc23)cc1 ; c1ccc(-c2cccc3c2oc2c(N(c4ccc(-c5ccc(-c6ccc7sc8ccccc8c7c6)cc5)cc4)c4ccc5c(c4)oc4ccccc45)cccc23)cc1]

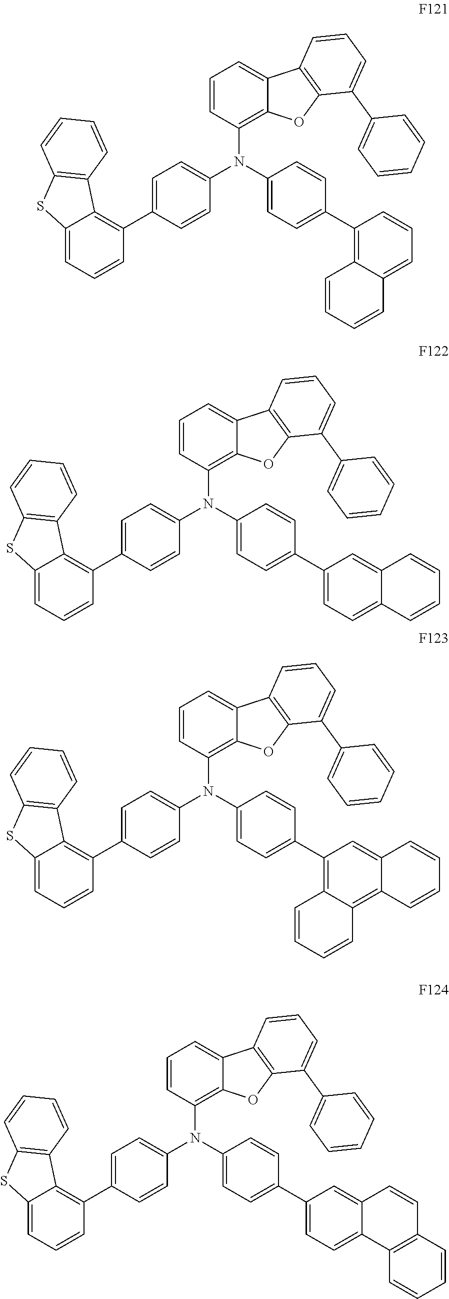 [SMILES: c1ccc(-c2cccc3c2oc2c(N(c4ccc(-c5cccc6ccccc56)cc4)c4ccc(-c5cccc6sc7ccccc7c56)cc4)cccc23)cc1 ; c1ccc(-c2cccc3c2oc2c(N(c4ccc(-c5ccc6ccccc6c5)cc4)c4ccc(-c5cccc6sc7ccccc7c56)cc4)cccc23)cc1 ; c1ccc(-c2cccc3c2oc2c(N(c4ccc(-c5cc6ccccc6c6ccccc56)cc4)c4ccc(-c5cccc6sc7ccccc7c56)cc4)cccc23)cc1 ; c1ccc(-c2cccc3c2oc2c(N(c4ccc(-c5ccc6c(ccc7ccccc76)c5)cc4)c4ccc(-c5cccc6sc7ccccc7c56)cc4)cccc23)cc1]

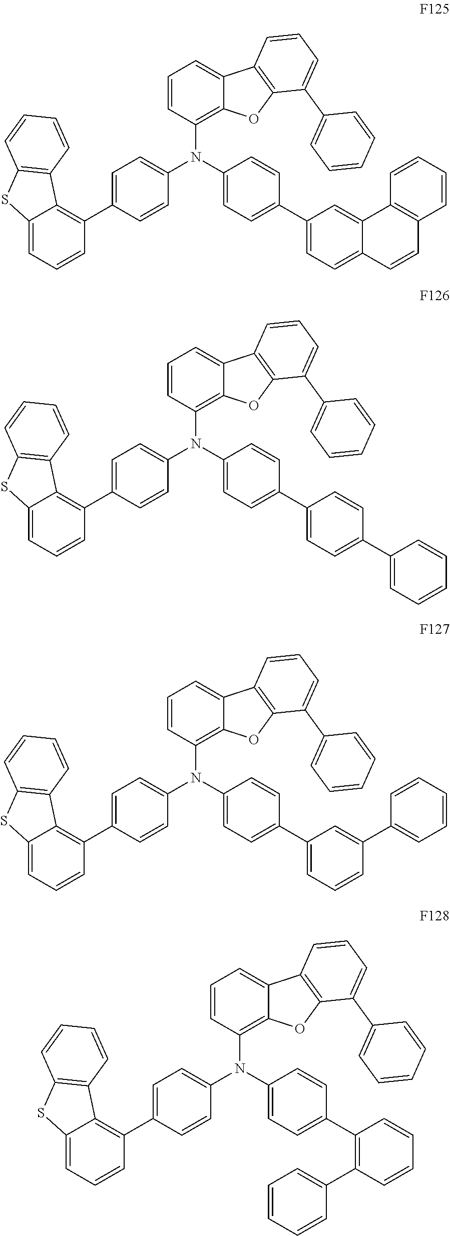 [SMILES: c1ccc(-c2cccc3c2oc2c(N(c4ccc(-c5ccc6ccc7ccccc7c6c5)cc4)c4ccc(-c5cccc6sc7ccccc7c56)cc4)cccc23)cc1 ; c1ccc(-c2ccc(-c3ccc(N(c4ccc(-c5cccc6sc7ccccc7c56)cc4)c4cccc5c4oc4c(-c6ccccc6)cccc45)cc3)cc2)cc1 ; c1ccc(-c2cccc(-c3ccc(N(c4ccc(-c5cccc6sc7ccccc7c56)cc4)c4cccc5c4oc4c(-c6ccccc6)cccc45)cc3)c2)cc1 ; c1ccc(-c2ccccc2-c2ccc(N(c3ccc(-c4cccc5sc6ccccc6c45)cc3)c3cccc4c3oc3c(-c5ccccc5)cccc34)cc2)cc1]

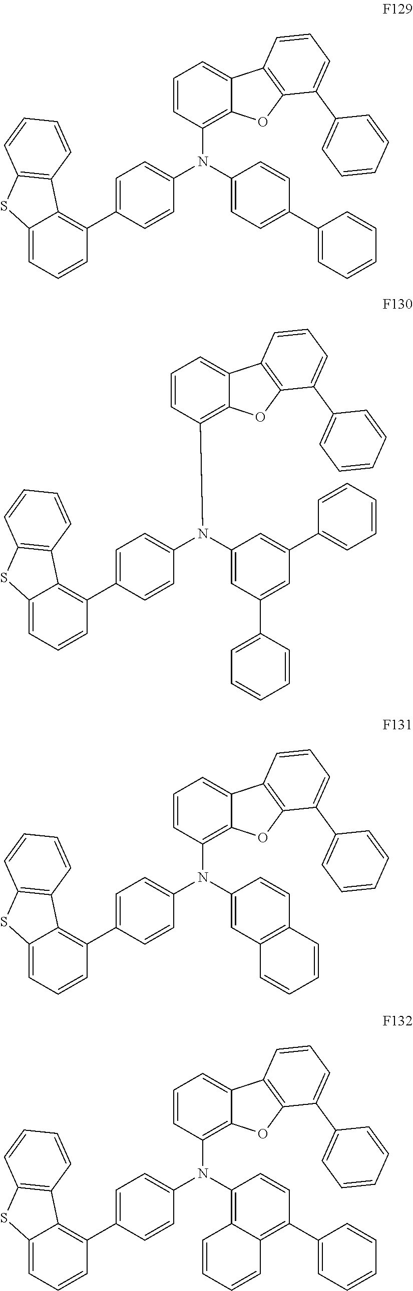 [SMILES: c1ccc(-c2ccc(N(c3ccc(-c4cccc5sc6ccccc6c45)cc3)c3cccc4c3oc3c(-c5ccccc5)cccc34)cc2)cc1 ; c1ccc(-c2cc(-c3ccccc3)cc(N(c3ccc(-c4cccc5sc6ccccc6c45)cc3)c3cccc4c3oc3c(-c5ccccc5)cccc34)c2)cc1 ; c1ccc(-c2cccc3c2oc2c(N(c4ccc(-c5cccc6sc7ccccc7c56)cc4)c4ccc5ccccc5c4)cccc23)cc1 ; c1ccc(-c2ccc(N(c3ccc(-c4cccc5sc6ccccc6c45)cc3)c3cccc4c3oc3c(-c5ccccc5)cccc34)c3ccccc23)cc1]

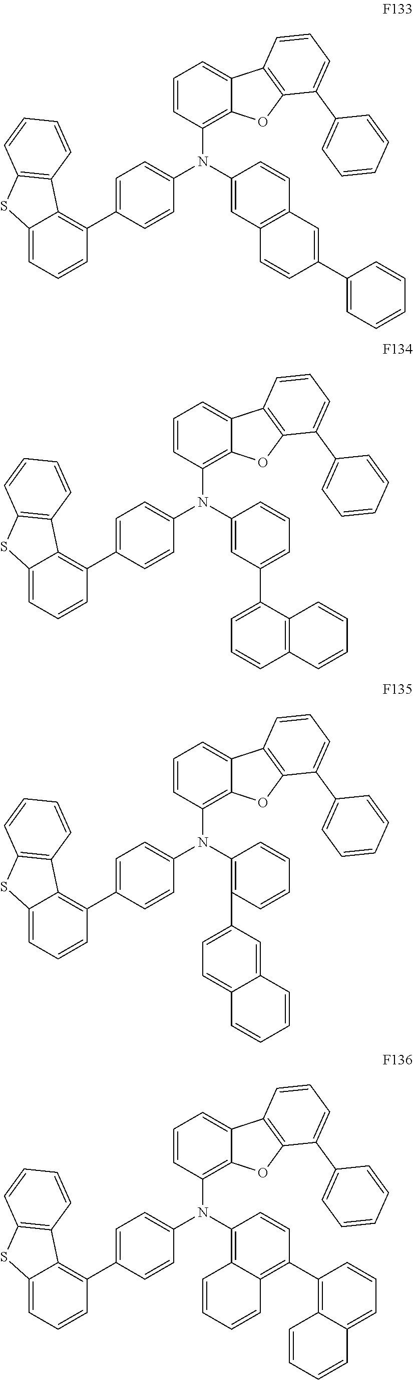

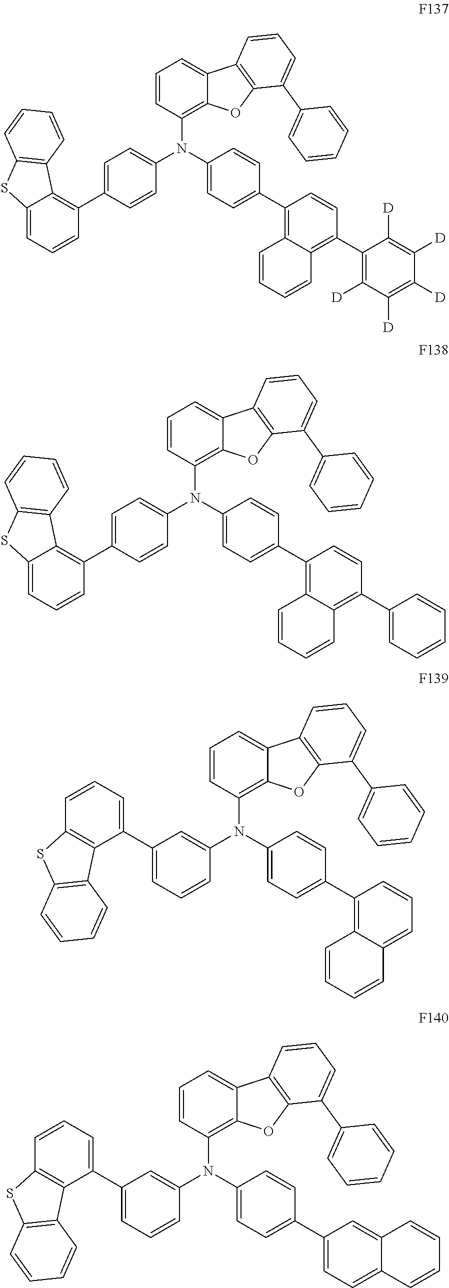 [SMILES: [2H]c1c([2H])c([2H])c(-c2ccc(-c3ccc(N(c4ccc(-c5cccc6sc7ccccc7c56)cc4)c4cccc5c4oc4c(-c6ccccc6)cccc45)cc3)c3ccccc23)c([2H])c1[2H] ; c1ccc(-c2ccc(-c3ccc(N(c4ccc(-c5cccc6sc7ccccc7c56)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5cccc6sc7ccccc7c56)c4)cccc23)cc1 ; c1ccc(-c2cccc3c2oc2c(N(c4ccc(-c5ccc6ccccc6c5)cc4)c4cccc(-c5cccc6sc7ccccc7c56)c4)cccc23)cc1]

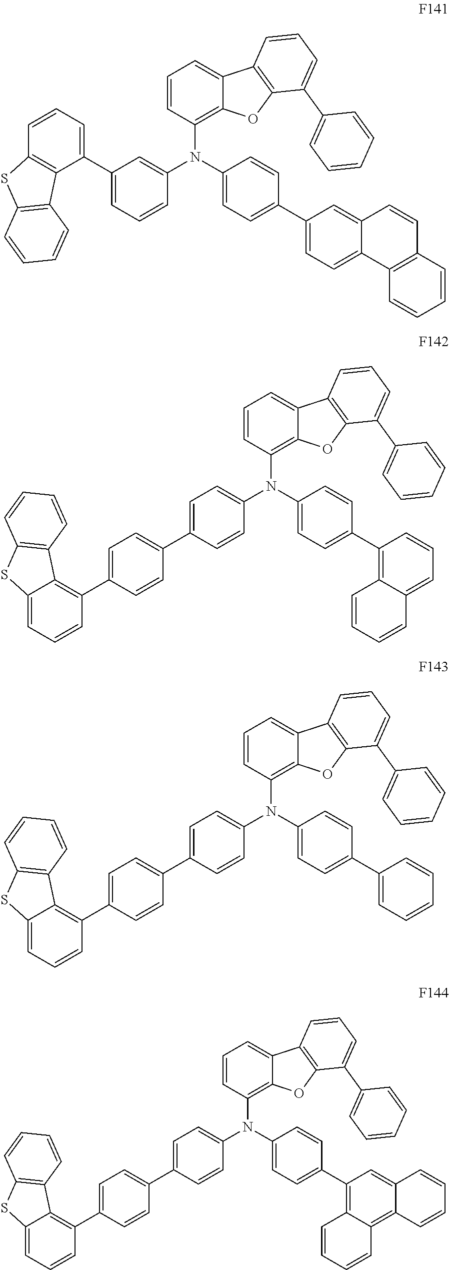 [SMILES: c1ccc(-c2cccc3c2oc2c(N(c4ccc(-c5ccc6c(ccc7ccccc76)c5)cc4)c4cccc(-c5cccc6sc7ccccc7c56)c4)cccc23)cc1 ; c1ccc(-c2cccc3c2oc2c(N(c4ccc(-c5ccc(-c6cccc7sc8ccccc8c67)cc5)cc4)c4ccc(-c5cccc6ccccc56)cc4)cccc23)cc1 ; c1ccc(-c2ccc(N(c3ccc(-c4ccc(-c5cccc6sc7ccccc7c56)cc4)cc3)c3cccc4c3oc3c(-c5ccccc5)cccc34)cc2)cc1 ; c1ccc(-c2cccc3c2oc2c(N(c4ccc(-c5ccc(-c6cccc7sc8ccccc8c67)cc5)cc4)c4ccc(-c5cc6ccccc6c6ccccc56)cc4)cccc23)cc1]

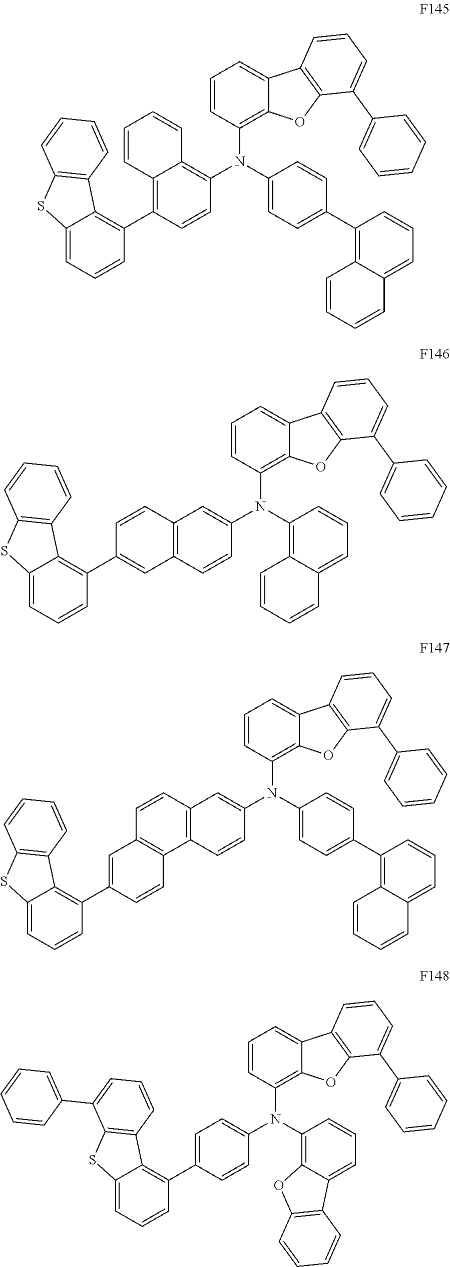 [SMILES: c1ccc(-c2cccc3c2oc2c(N(c4ccc(-c5cccc6ccccc56)cc4)c4ccc(-c5cccc6sc7ccccc7c56)c5ccccc45)cccc23)cc1 ; c1ccc(-c2cccc3c2oc2c(N(c4ccc5cc(-c6cccc7sc8ccccc8c67)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7cccc8sc9ccccc9c78)ccc65)c4)cccc23)cc1 ; c1ccc(-c2cccc3c2oc2c(N(c4ccc(-c5cccc6sc7c(-c8ccccc8)cccc7c56)cc4)c4cccc5c4oc4ccccc45)cccc23)cc1]

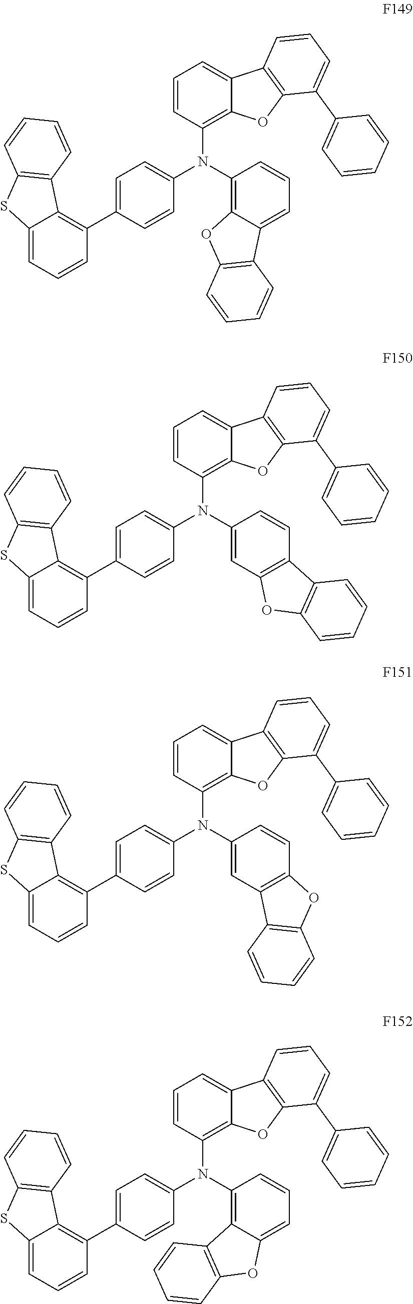 [SMILES: c1ccc(-c2cccc3c2oc2c(N(c4ccc(-c5cccc6sc7ccccc7c56)cc4)c4cccc5c4oc4ccccc45)cccc23)cc1 ; c1ccc(-c2cccc3c2oc2c(N(c4ccc(-c5cccc6sc7ccccc7c56)cc4)c4ccc5c(c4)oc4ccccc45)cccc23)cc1 ; c1ccc(-c2cccc3c2oc2c(N(c4ccc(-c5cccc6sc7ccccc7c56)cc4)c4ccc5oc6ccccc6c5c4)cccc23)cc1 ; c1ccc(-c2cccc3c2oc2c(N(c4ccc(-c5cccc6sc7ccccc7c56)cc4)c4cccc5oc6ccccc6c45)cccc23)cc1]

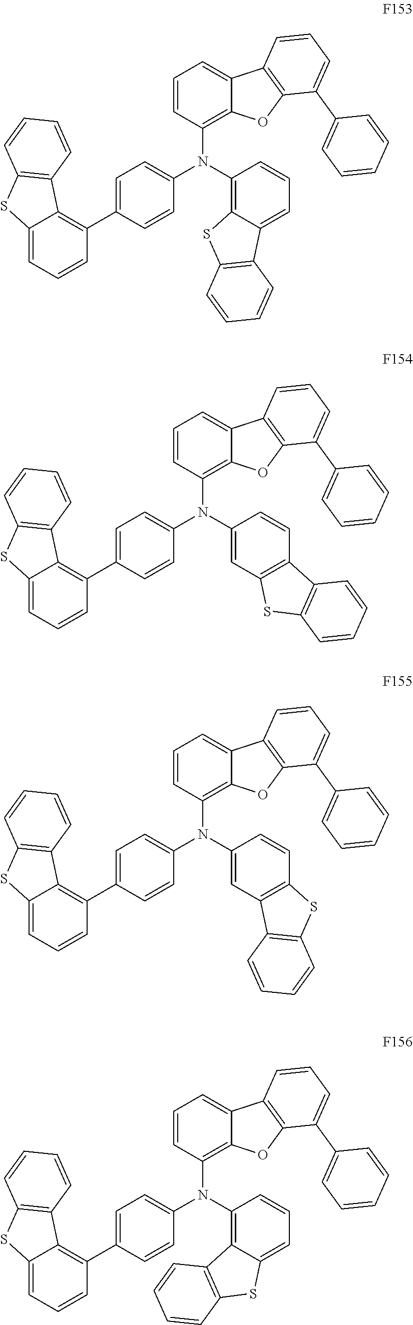 [SMILES: c1ccc(-c2cccc3c2oc2c(N(c4ccc(-c5cccc6sc7ccccc7c56)cc4)c4cccc5c4sc4ccccc45)cccc23)cc1 ; c1ccc(-c2cccc3c2oc2c(N(c4ccc(-c5cccc6sc7ccccc7c56)cc4)c4ccc5c(c4)sc4ccccc45)cccc23)cc1 ; c1ccc(-c2cccc3c2oc2c(N(c4ccc(-c5cccc6sc7ccccc7c56)cc4)c4ccc5sc6ccccc6c5c4)cccc23)cc1 ; c1ccc(-c2cccc3c2oc2c(N(c4ccc(-c5cccc6sc7ccccc7c56)cc4)c4cccc5sc6ccccc6c45)cccc23)cc1]

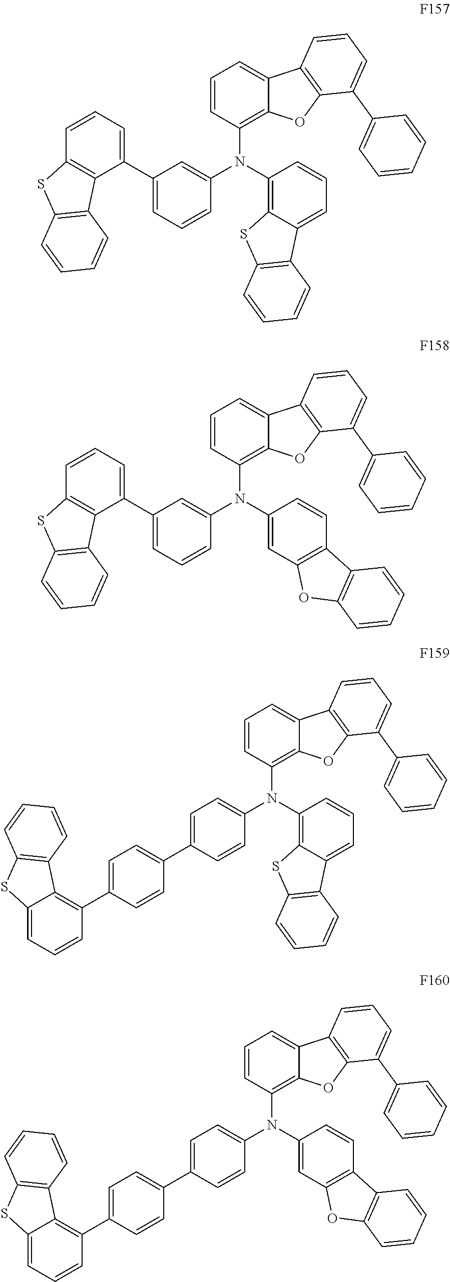 [SMILES: c1ccc(-c2cccc3c2oc2c(N(c4cccc(-c5cccc6sc7ccccc7c56)c4)c4cccc5c4sc4ccccc45)cccc23)cc1 ; c1ccc(-c2cccc3c2oc2c(N(c4cccc(-c5cccc6sc7ccccc7c56)c4)c4ccc5c(c4)oc4ccccc45)cccc23)cc1 ; c1ccc(-c2cccc3c2oc2c(N(c4ccc(-c5ccc(-c6cccc7sc8ccccc8c67)cc5)cc4)c4cccc5c4sc4ccccc45)cccc23)cc1 ; c1ccc(-c2cccc3c2oc2c(N(c4ccc(-c5ccc(-c6cccc7sc8ccccc8c67)cc5)cc4)c4ccc5c(c4)oc4ccccc45)cccc23)cc1]

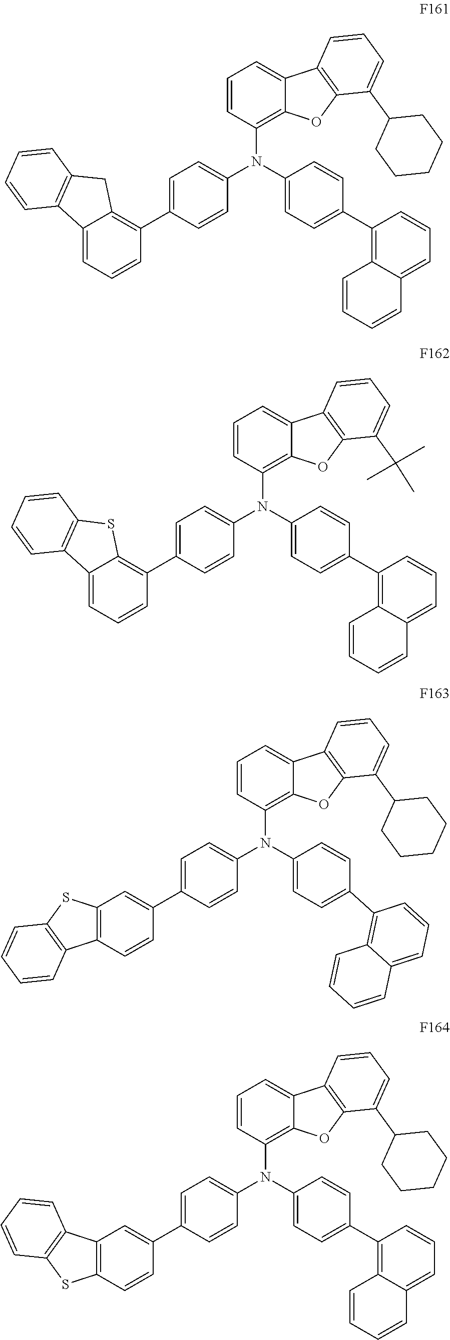 [SMILES: c1ccc2c(c1)Cc1c(-c3ccc(N(c4ccc(-c5cccc6ccccc56)cc4)c4cccc5c4oc4c(C6CCCCC6)cccc45)cc3)cccc1-2 ; CC(C)(C)c1cccc2c1oc1c(N(c3ccc(-c4cccc5ccccc45)cc3)c3ccc(-c4cccc5c4sc4ccccc45)cc3)cccc12 ; c1ccc2c(-c3ccc(N(c4ccc(-c5ccc6c(c5)sc5ccccc56)cc4)c4cccc5c4oc4c(C6CCCCC6)cccc45)cc3)cccc2c1 ; c1ccc2c(-c3ccc(N(c4ccc(-c5ccc6sc7ccccc7c6c5)cc4)c4cccc5c4oc4c(C6CCCCC6)cccc45)cc3)cccc2c1]

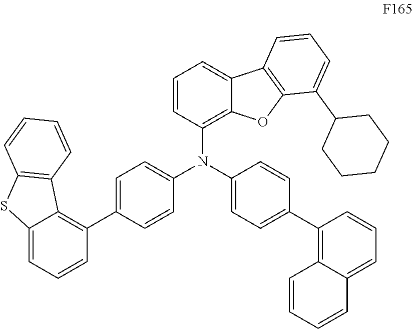 [SMILES: c1ccc2c(-c3ccc(N(c4ccc(-c5cccc6sc7ccccc7c56)cc4)c4cccc5c4oc4c(C6CCCCC6)cccc45)cc3)cccc2c1]

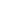

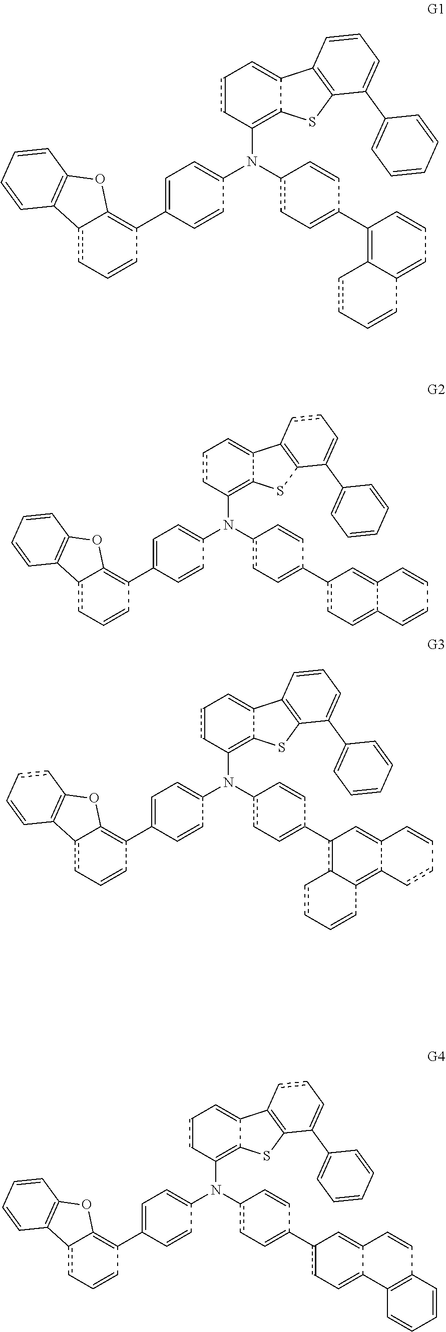

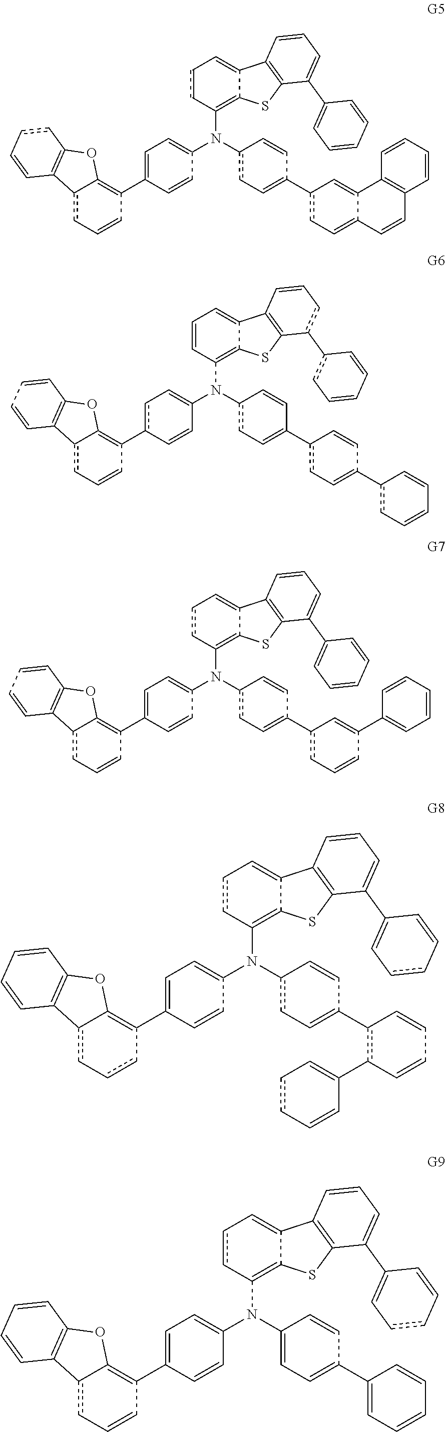

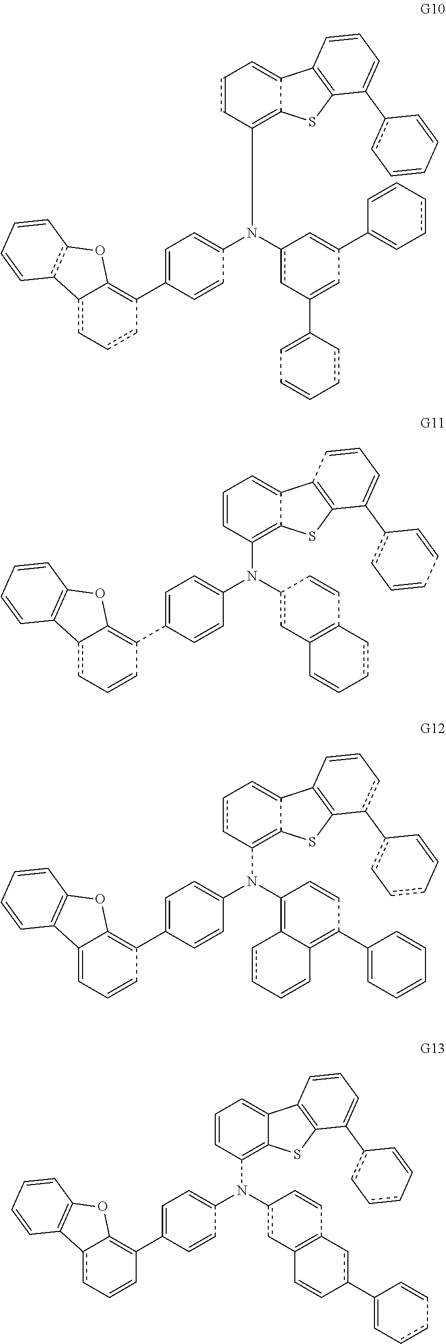

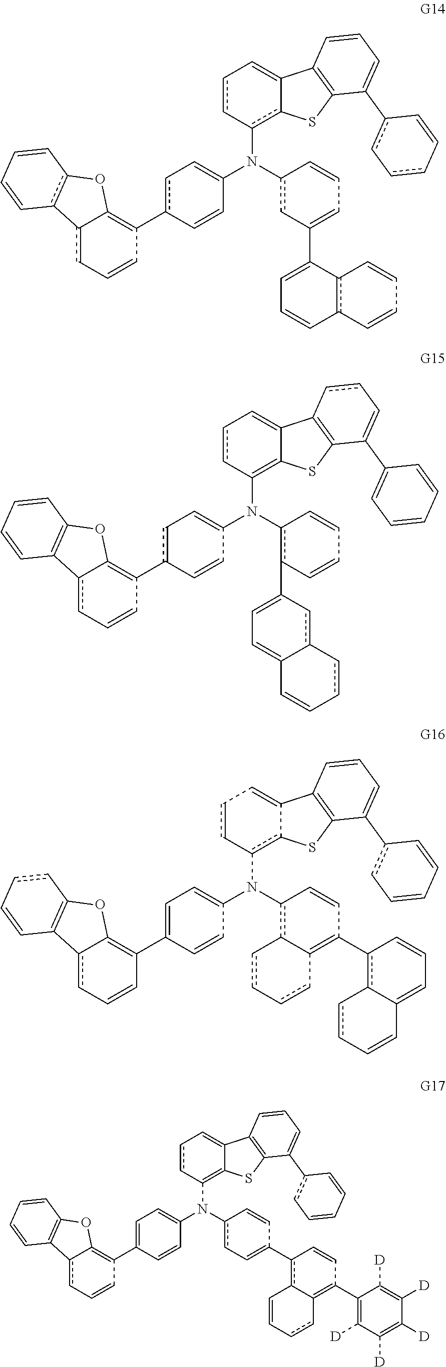

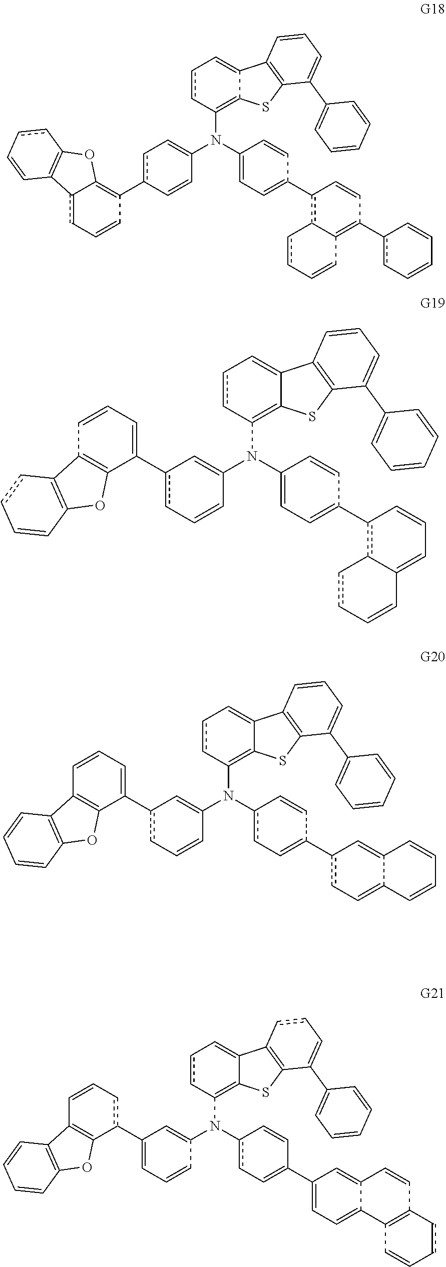

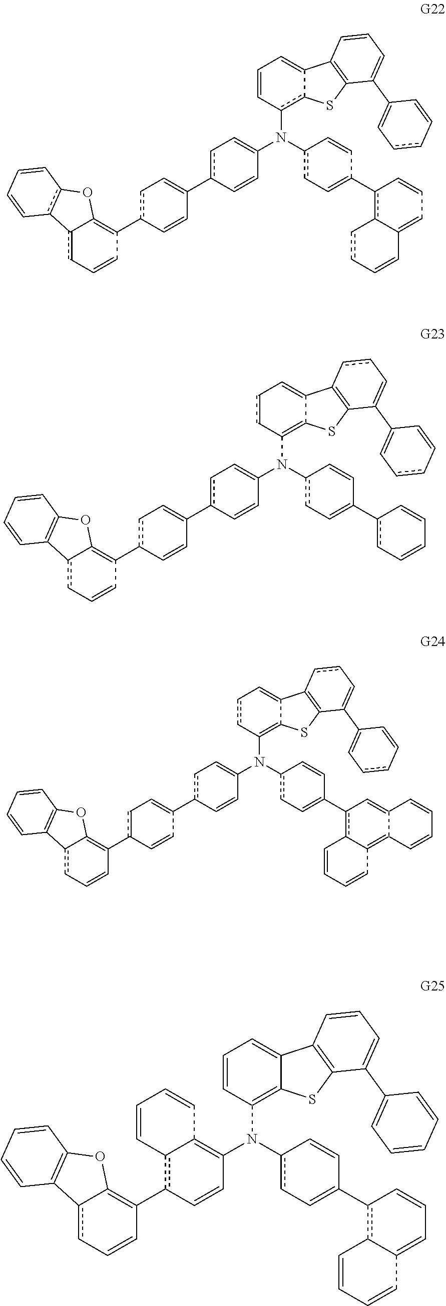

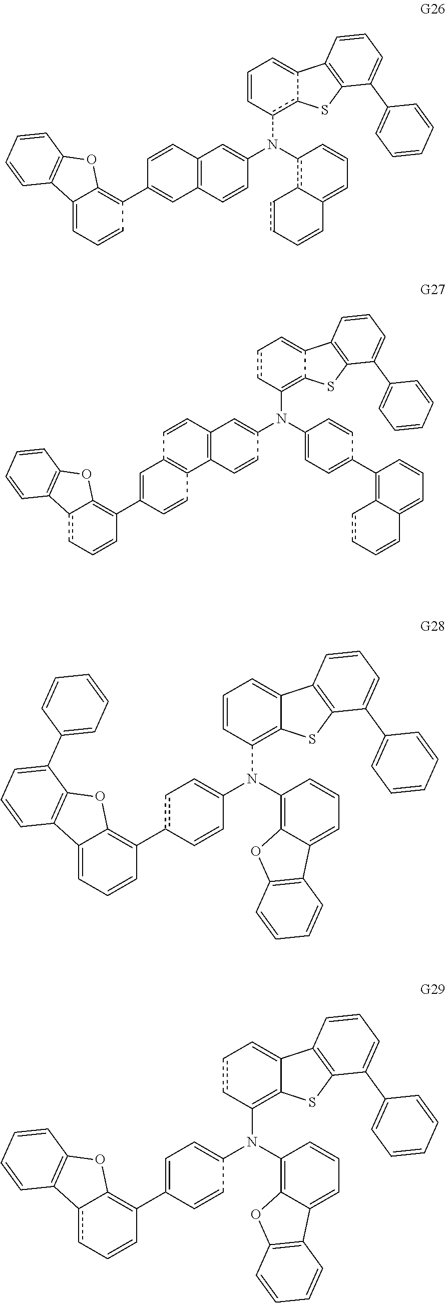

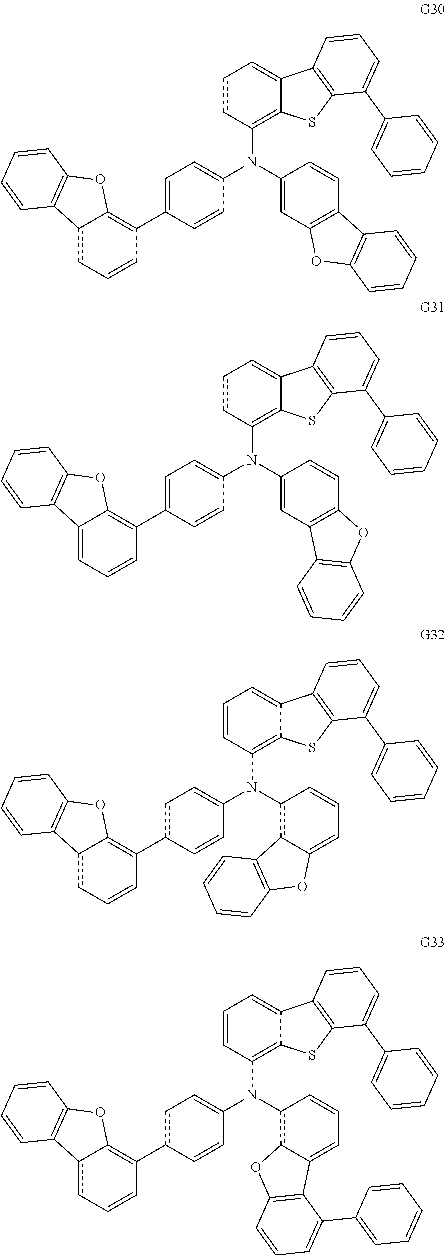

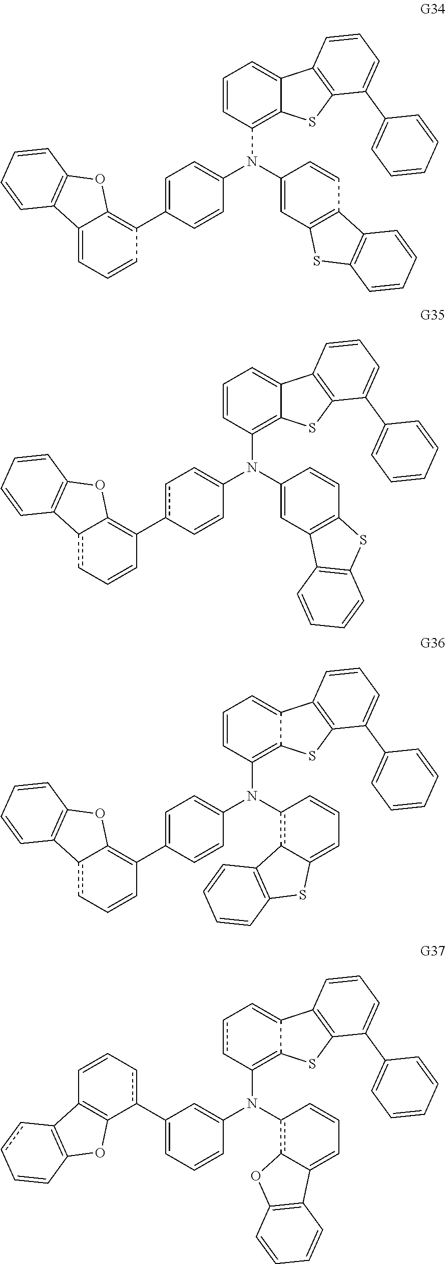

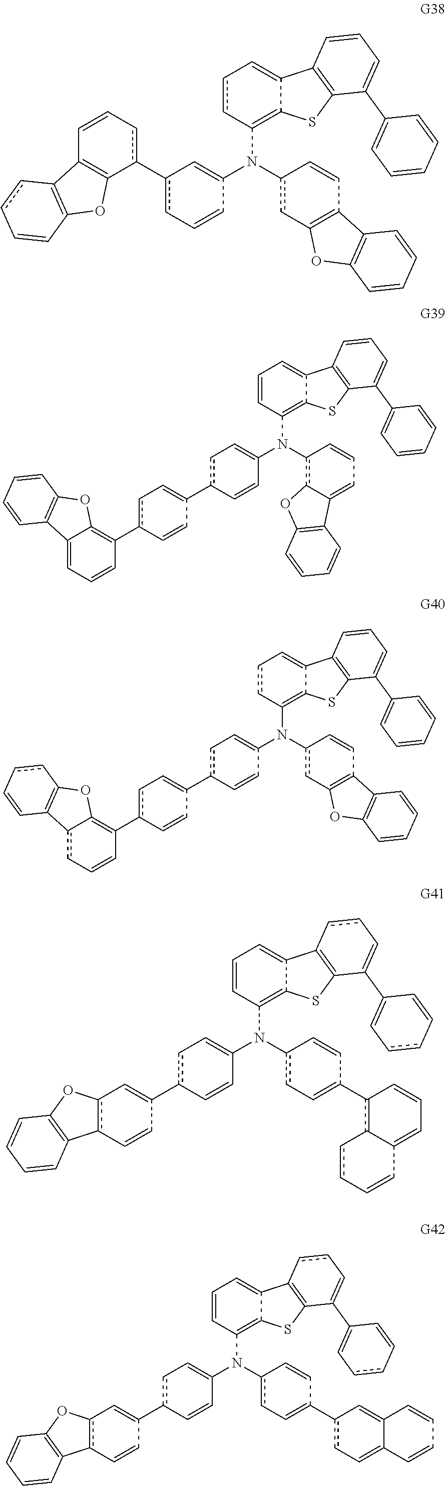

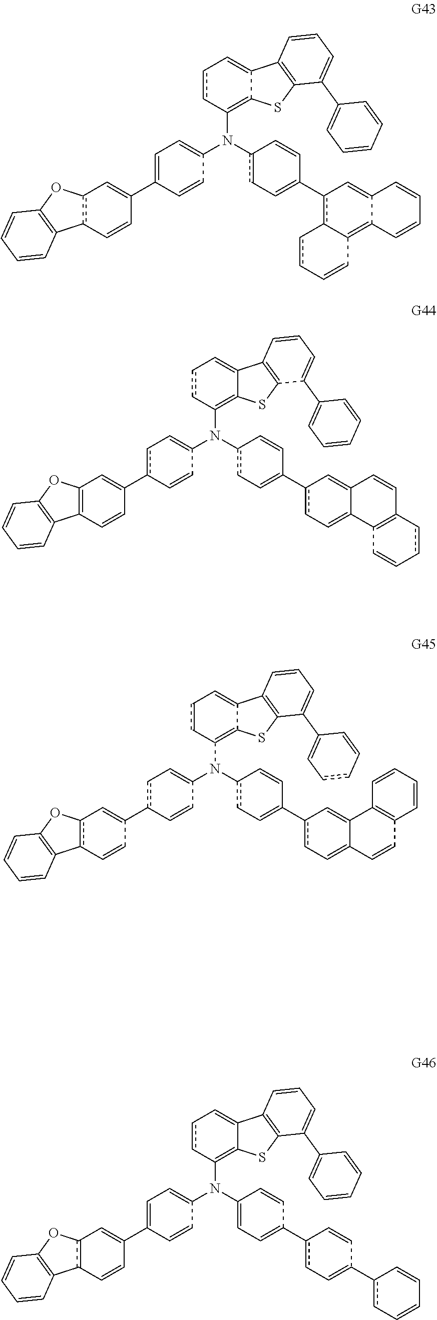

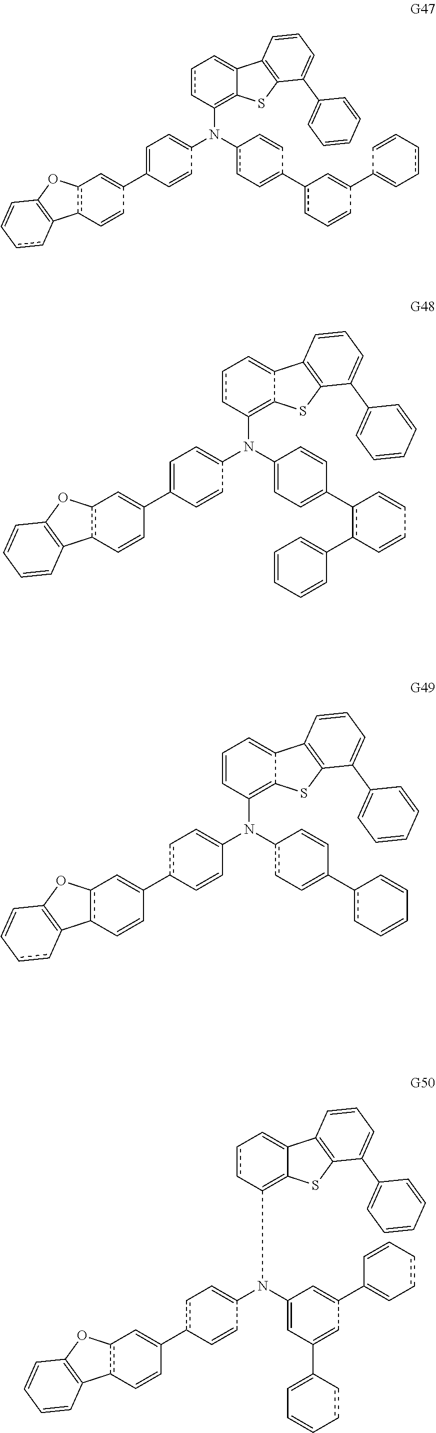

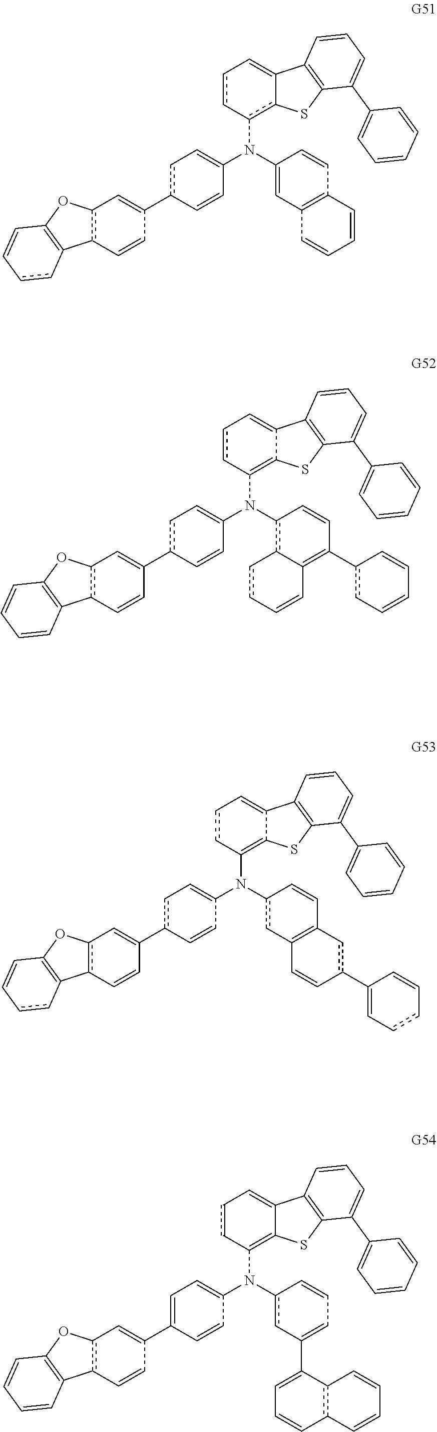

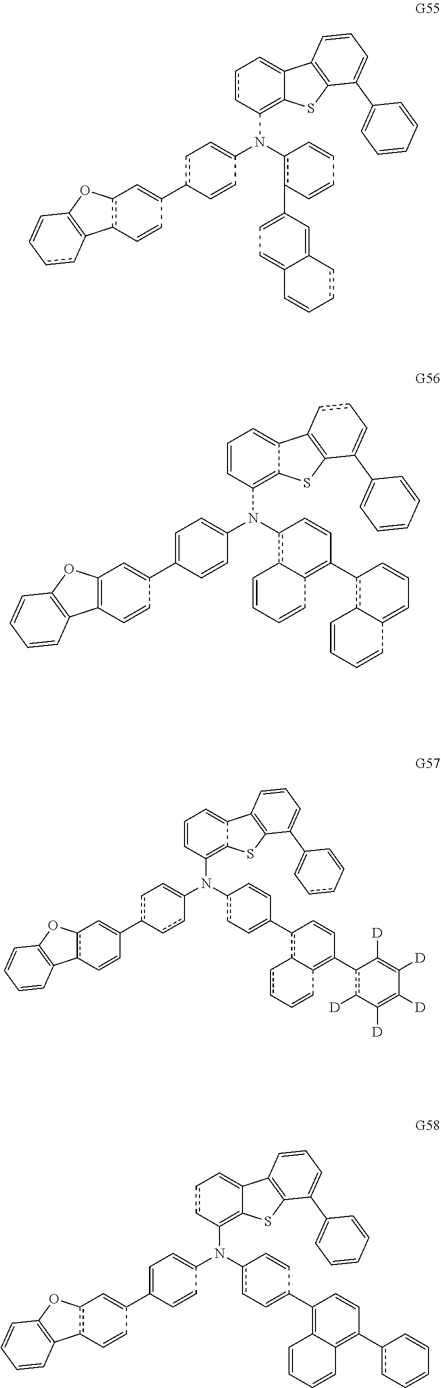

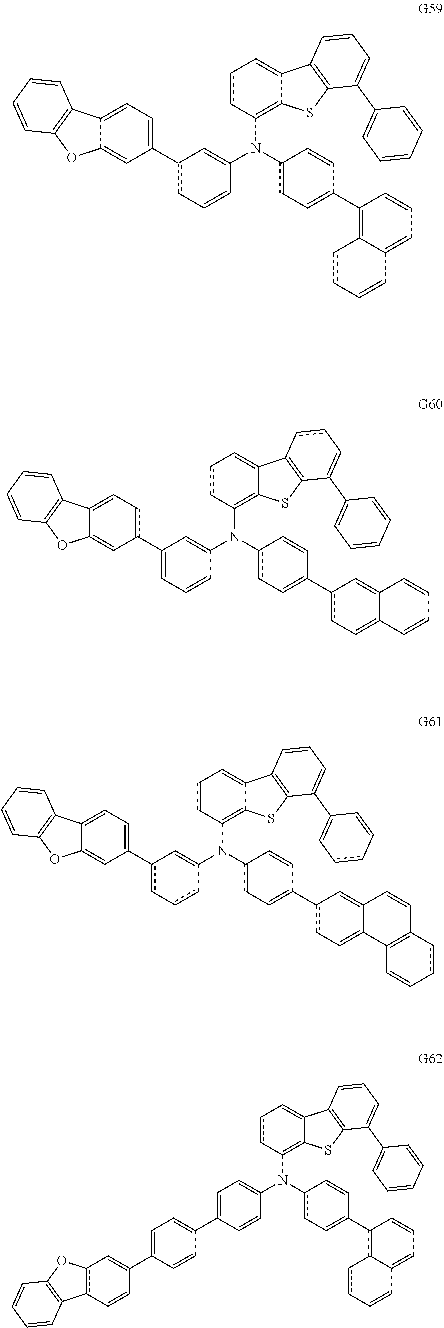

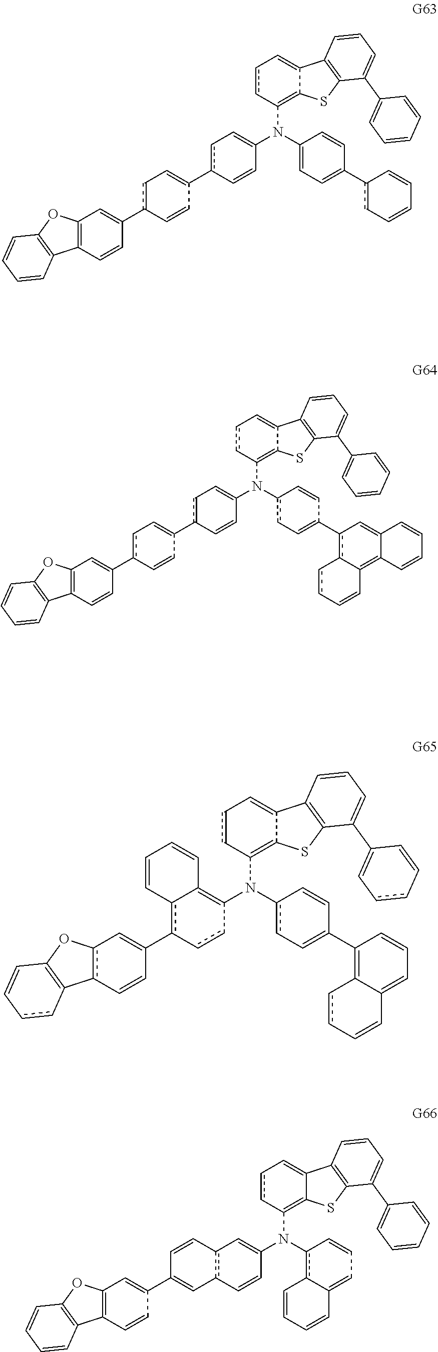

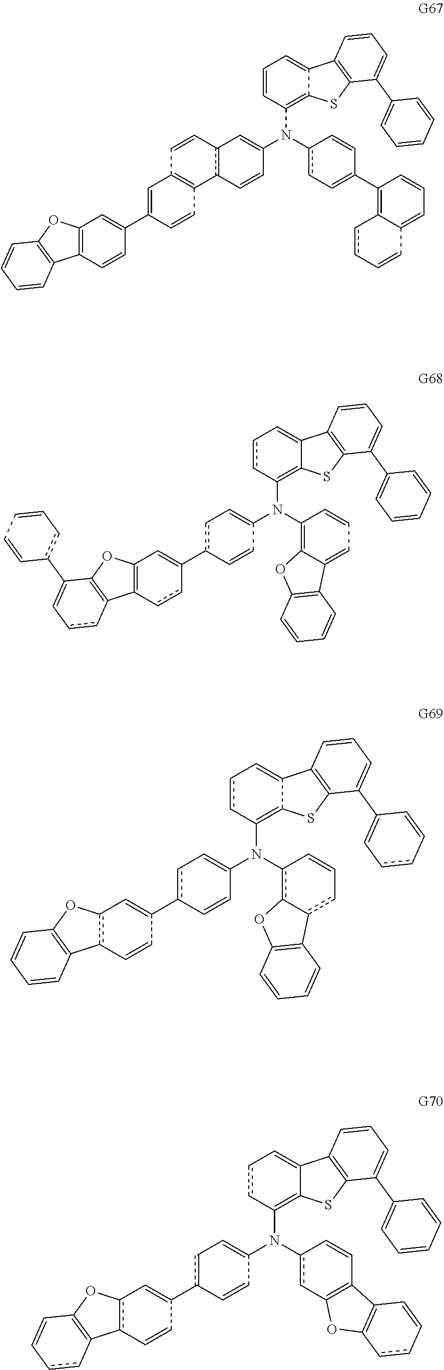

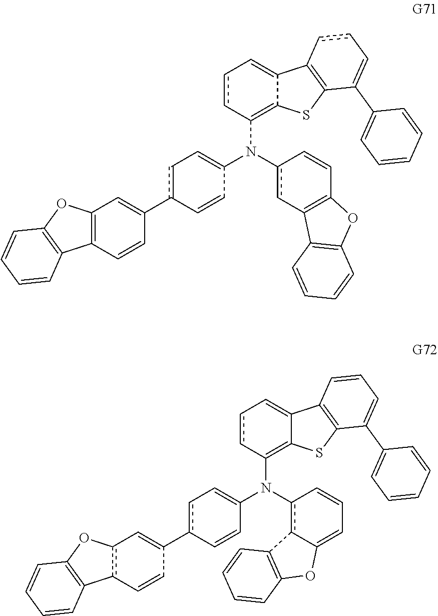

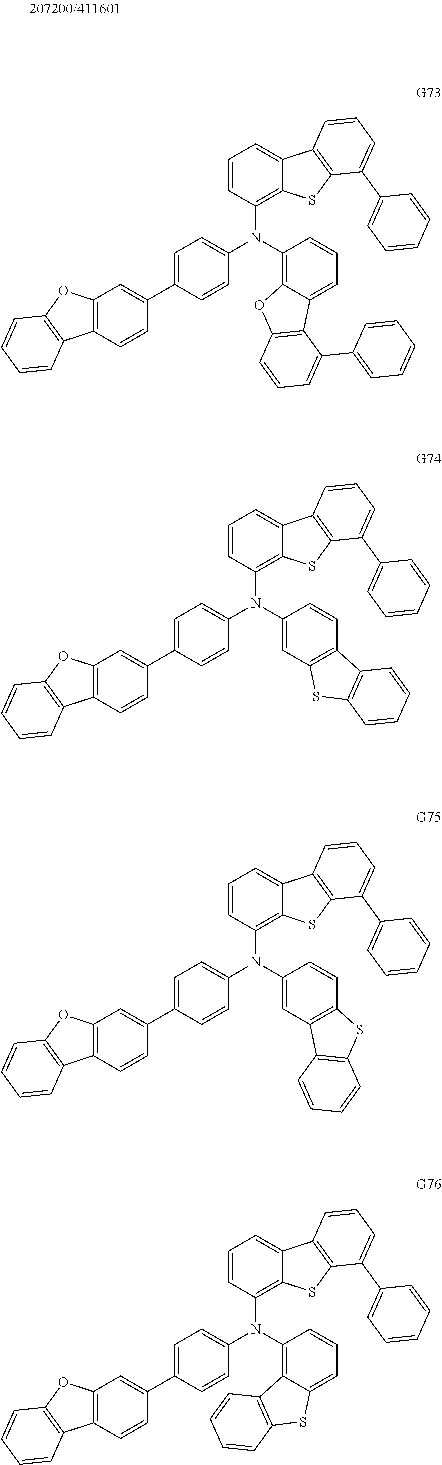 [SMILES: c1ccc(-c2cccc3c2sc2c(N(c4ccc(-c5ccc6c(c5)oc5ccccc56)cc4)c4cccc5c4oc4cccc(-c6ccccc6)c45)cccc23)cc1 ; c1ccc(-c2cccc3c2sc2c(N(c4ccc(-c5ccc6c(c5)oc5ccccc56)cc4)c4ccc5c(c4)sc4ccccc45)cccc23)cc1 ; c1ccc(-c2cccc3c2sc2c(N(c4ccc(-c5ccc6c(c5)oc5ccccc56)cc4)c4ccc5sc6ccccc6c5c4)cccc23)cc1 ; c1ccc(-c2cccc3c2sc2c(N(c4ccc(-c5ccc6c(c5)oc5ccccc56)cc4)c4cccc5sc6ccccc6c45)cccc23)cc1]

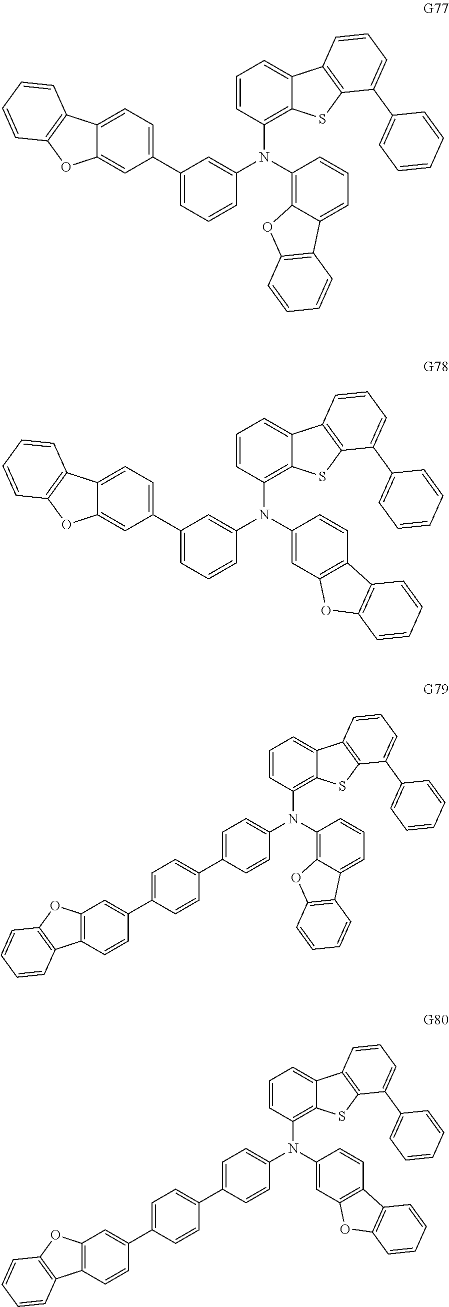 [SMILES: c1ccc(-c2cccc3c2sc2c(N(c4cccc(-c5ccc6c(c5)oc5ccccc56)c4)c4cccc5c4oc4ccccc45)cccc23)cc1 ; c1ccc(-c2cccc3c2sc2c(N(c4cccc(-c5ccc6c(c5)oc5ccccc56)c4)c4ccc5c(c4)oc4ccccc45)cccc23)cc1 ; c1ccc(-c2cccc3c2sc2c(N(c4ccc(-c5ccc(-c6ccc7c(c6)oc6ccccc67)cc5)cc4)c4cccc5c4oc4ccccc45)cccc23)cc1 ; c1ccc(-c2cccc3c2sc2c(N(c4ccc(-c5ccc(-c6ccc7c(c6)oc6ccccc67)cc5)cc4)c4ccc5c(c4)oc4ccccc45)cccc23)cc1]

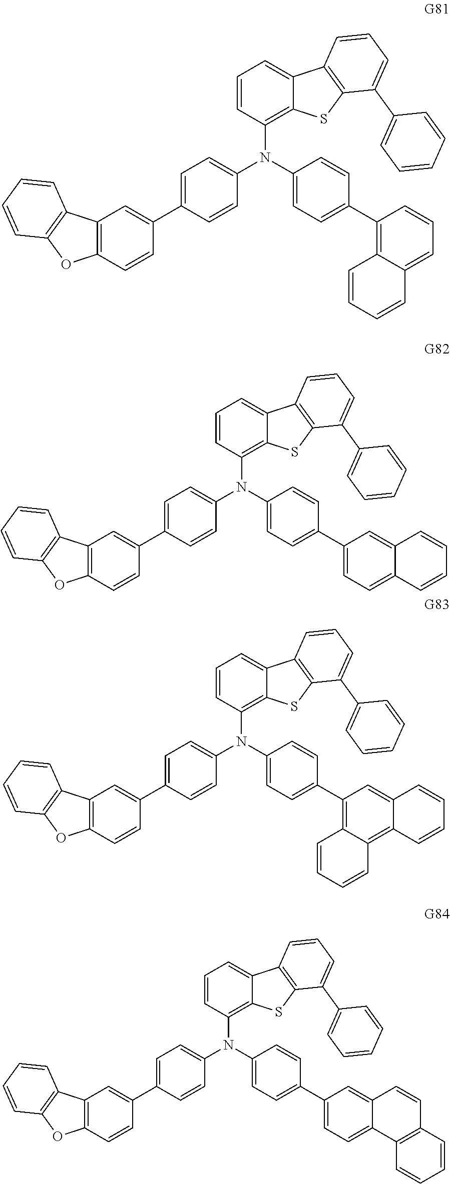 [SMILES: c1ccc(-c2cccc3c2sc2c(N(c4ccc(-c5ccc6oc7ccccc7c6c5)cc4)c4ccc(-c5cccc6ccccc56)cc4)cccc23)cc1 ; c1ccc(-c2cccc3c2sc2c(N(c4ccc(-c5ccc6ccccc6c5)cc4)c4ccc(-c5ccc6oc7ccccc7c6c5)cc4)cccc23)cc1 ; c1ccc(-c2cccc3c2sc2c(N(c4ccc(-c5ccc6oc7ccccc7c6c5)cc4)c4ccc(-c5cc6ccccc6c6ccccc56)cc4)cccc23)cc1 ; c1ccc(-c2cccc3c2sc2c(N(c4ccc(-c5ccc6c(ccc7ccccc76)c5)cc4)c4ccc(-c5ccc6oc7ccccc7c6c5)cc4)cccc23)cc1]

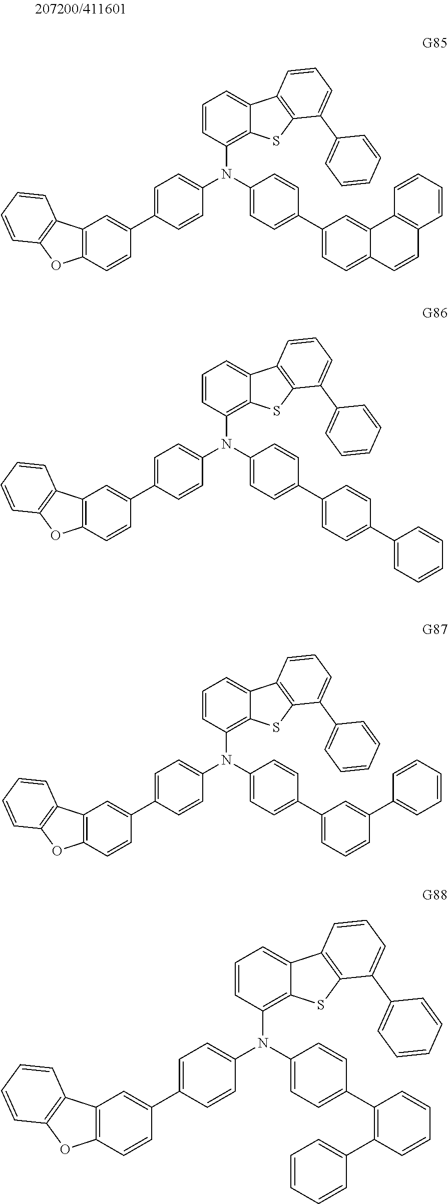 [SMILES: c1ccc(-c2cccc3c2sc2c(N(c4ccc(-c5ccc6ccc7ccccc7c6c5)cc4)c4ccc(-c5ccc6oc7ccccc7c6c5)cc4)cccc23)cc1 ; c1ccc(-c2ccc(-c3ccc(N(c4ccc(-c5ccc6oc7ccccc7c6c5)cc4)c4cccc5c4sc4c(-c6ccccc6)cccc45)cc3)cc2)cc1 ; c1ccc(-c2cccc(-c3ccc(N(c4ccc(-c5ccc6oc7ccccc7c6c5)cc4)c4cccc5c4sc4c(-c6ccccc6)cccc45)cc3)c2)cc1 ; c1ccc(-c2ccccc2-c2ccc(N(c3ccc(-c4ccc5oc6ccccc6c5c4)cc3)c3cccc4c3sc3c(-c5ccccc5)cccc34)cc2)cc1]

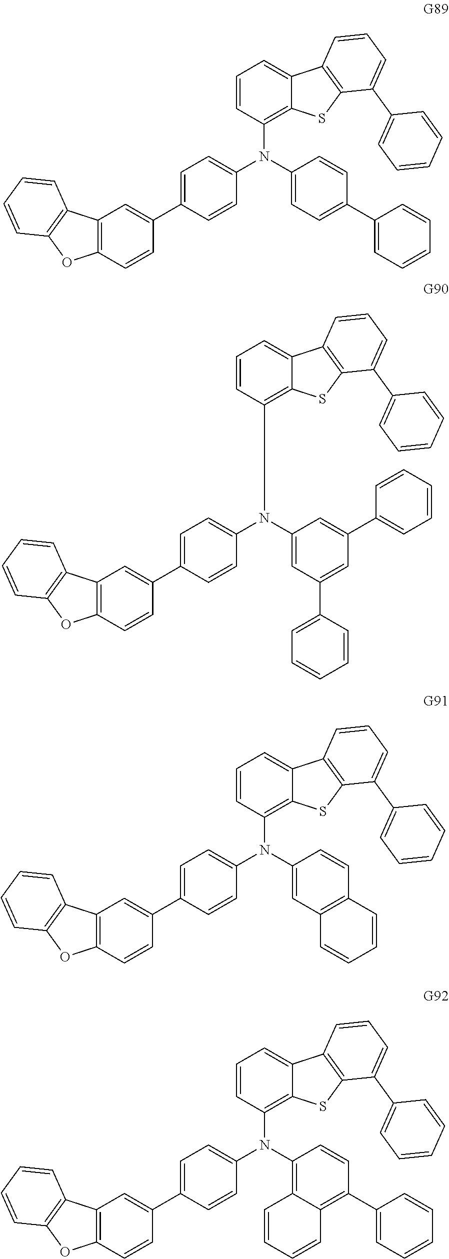 [SMILES: c1ccc(-c2ccc(N(c3ccc(-c4ccc5oc6ccccc6c5c4)cc3)c3cccc4c3sc3c(-c5ccccc5)cccc34)cc2)cc1 ; c1ccc(-c2cc(-c3ccccc3)cc(N(c3ccc(-c4ccc5oc6ccccc6c5c4)cc3)c3cccc4c3sc3c(-c5ccccc5)cccc34)c2)cc1 ; c1ccc(-c2cccc3c2sc2c(N(c4ccc(-c5ccc6oc7ccccc7c6c5)cc4)c4ccc5ccccc5c4)cccc23)cc1 ; c1ccc(-c2ccc(N(c3ccc(-c4ccc5oc6ccccc6c5c4)cc3)c3cccc4c3sc3c(-c5ccccc5)cccc34)c3ccccc23)cc1]

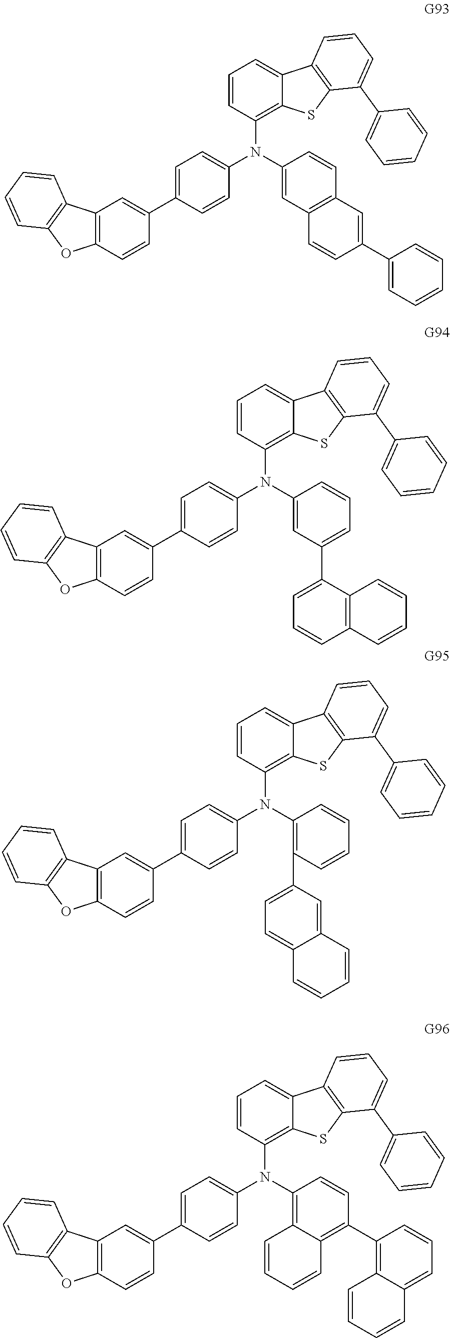

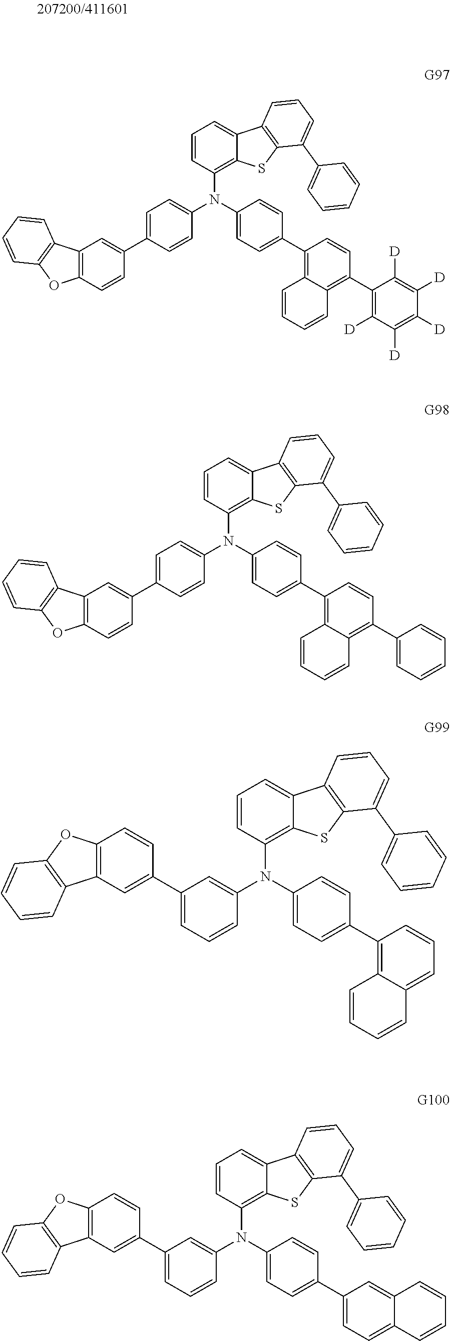 [SMILES: [2H]c1c([2H])c([2H])c(-c2ccc(-c3ccc(N(c4ccc(-c5ccc6oc7ccccc7c6c5)cc4)c4cccc5c4sc4c(-c6ccccc6)cccc45)cc3)c3ccccc23)c([2H])c1[2H] ; c1ccc(-c2ccc(-c3ccc(N(c4ccc(-c5ccc6oc7ccccc7c6c5)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4cccc(-c5ccc6oc7ccccc7c6c5)c4)cccc23)cc1 ; c1ccc(-c2cccc3c2sc2c(N(c4ccc(-c5ccc6ccccc6c5)cc4)c4cccc(-c5ccc6oc7ccccc7c6c5)c4)cccc23)cc1]

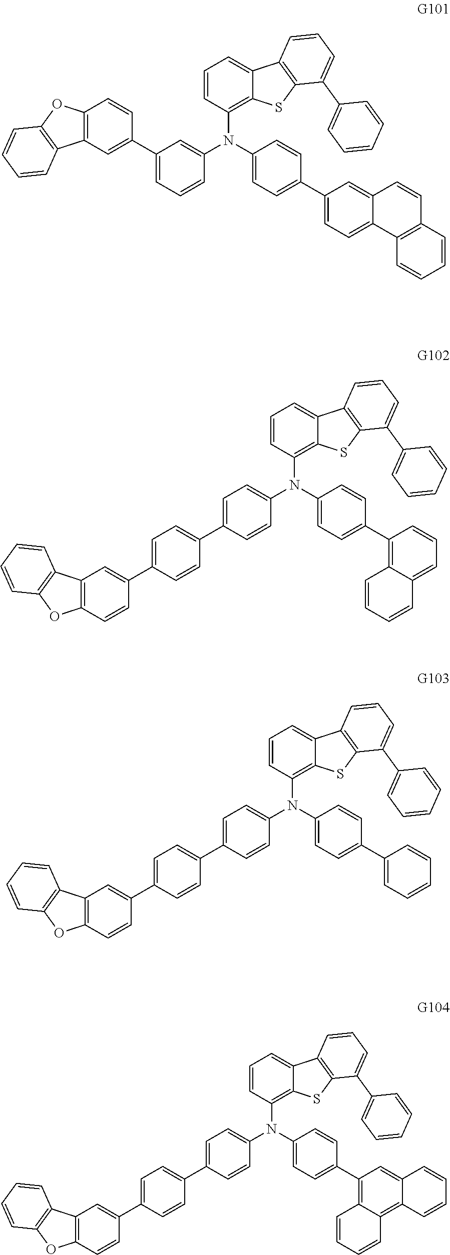 [SMILES: c1ccc(-c2cccc3c2sc2c(N(c4ccc(-c5ccc6c(ccc7ccccc76)c5)cc4)c4cccc(-c5ccc6oc7ccccc7c6c5)c4)cccc23)cc1 ; c1ccc(-c2cccc3c2sc2c(N(c4ccc(-c5ccc(-c6ccc7oc8ccccc8c7c6)cc5)cc4)c4ccc(-c5cccc6ccccc56)cc4)cccc23)cc1 ; c1ccc(-c2ccc(N(c3ccc(-c4ccc(-c5ccc6oc7ccccc7c6c5)cc4)cc3)c3cccc4c3sc3c(-c5ccccc5)cccc34)cc2)cc1 ; c1ccc(-c2cccc3c2sc2c(N(c4ccc(-c5ccc(-c6ccc7oc8ccccc8c7c6)cc5)cc4)c4ccc(-c5cc6ccccc6c6ccccc56)cc4)cccc23)cc1]

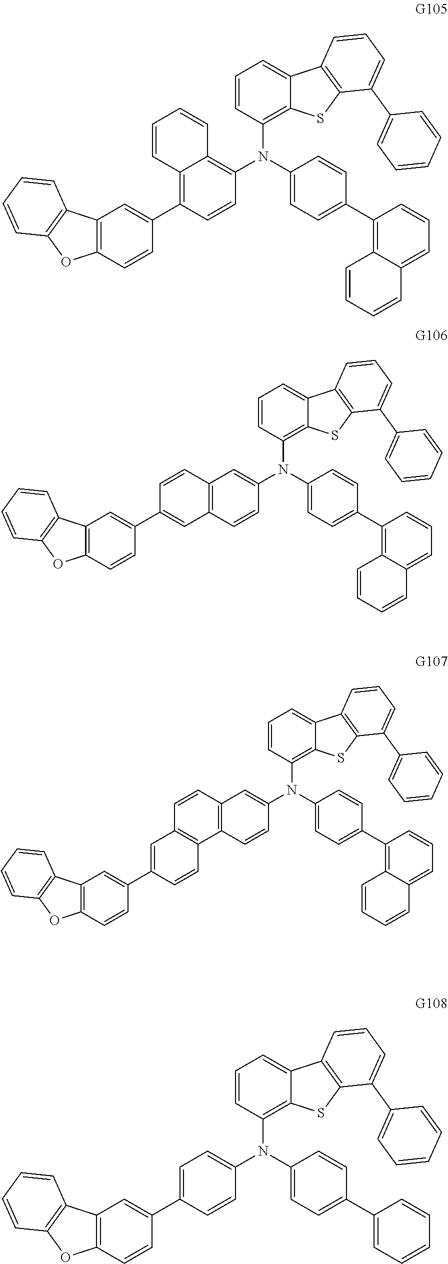 [SMILES: c1ccc(-c2cccc3c2sc2c(N(c4ccc(-c5cccc6ccccc56)cc4)c4ccc(-c5ccc6oc7ccccc7c6c5)c5ccccc45)cccc23)cc1 ; c1ccc(-c2cccc3c2sc2c(N(c4ccc(-c5cccc6ccccc56)cc4)c4ccc5cc(-c6ccc7oc8ccccc8c7c6)ccc5c4)cccc23)cc1 ; c1ccc(-c2cccc3c2sc2c(N(c4ccc(-c5cccc6ccccc56)cc4)c4ccc5c(ccc6cc(-c7ccc8oc9ccccc9c8c7)ccc65)c4)cccc23)cc1 ; c1ccc(-c2ccc(N(c3ccc(-c4ccc5oc6ccccc6c5c4)cc3)c3cccc4c3sc3c(-c5ccccc5)cccc34)cc2)cc1]

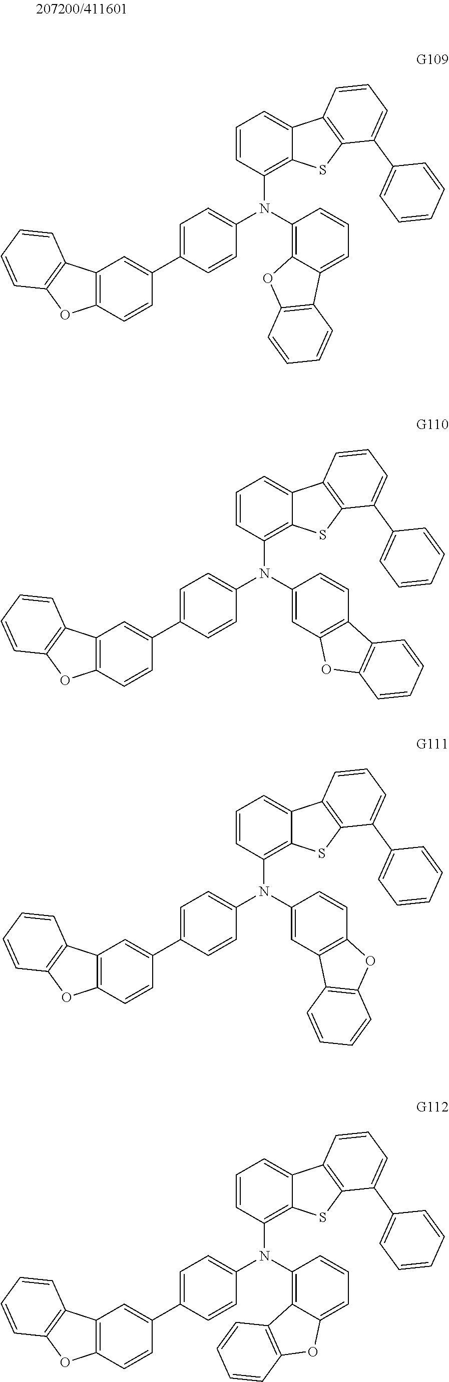 [SMILES: c1ccc(-c2cccc3c2sc2c(N(c4ccc(-c5ccc6oc7ccccc7c6c5)cc4)c4cccc5c4oc4ccccc45)cccc23)cc1 ; c1ccc(-c2cccc3c2sc2c(N(c4ccc(-c5ccc6oc7ccccc7c6c5)cc4)c4ccc5c(c4)oc4ccccc45)cccc23)cc1 ; c1ccc(-c2cccc3c2sc2c(N(c4ccc(-c5ccc6oc7ccccc7c6c5)cc4)c4ccc5oc6ccccc6c5c4)cccc23)cc1 ; c1ccc(-c2cccc3c2sc2c(N(c4ccc(-c5ccc6oc7ccccc7c6c5)cc4)c4cccc5oc6ccccc6c45)cccc23)cc1]

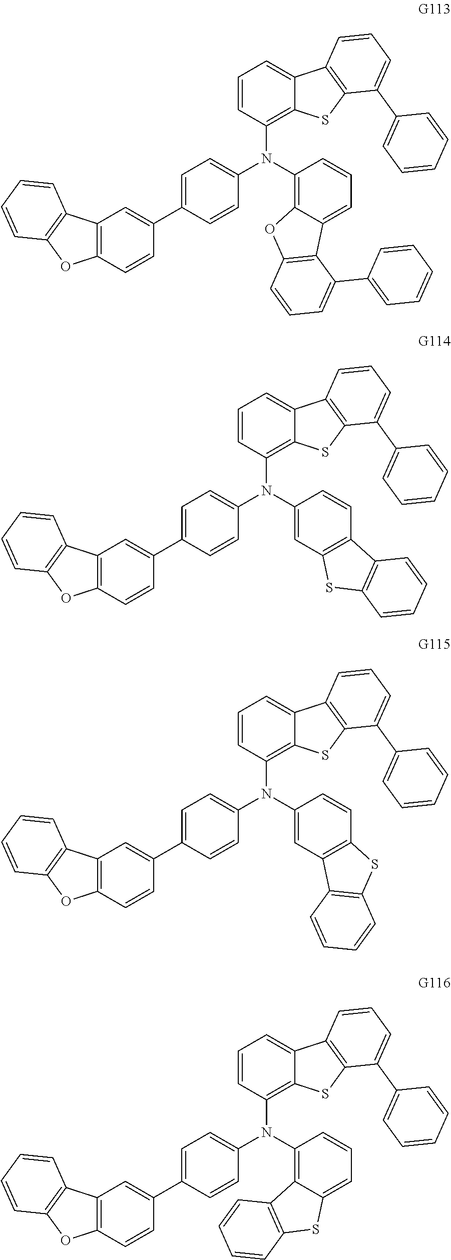 [SMILES: c1ccc(-c2cccc3c2sc2c(N(c4ccc(-c5ccc6oc7ccccc7c6c5)cc4)c4cccc5c4oc4cccc(-c6ccccc6)c45)cccc23)cc1 ; c1ccc(-c2cccc3c2sc2c(N(c4ccc(-c5ccc6oc7ccccc7c6c5)cc4)c4ccc5c(c4)sc4ccccc45)cccc23)cc1 ; c1ccc(-c2cccc3c2sc2c(N(c4ccc(-c5ccc6oc7ccccc7c6c5)cc4)c4ccc5sc6ccccc6c5c4)cccc23)cc1 ; c1ccc(-c2cccc3c2sc2c(N(c4ccc(-c5ccc6oc7ccccc7c6c5)cc4)c4cccc5sc6ccccc6c45)cccc23)cc1]

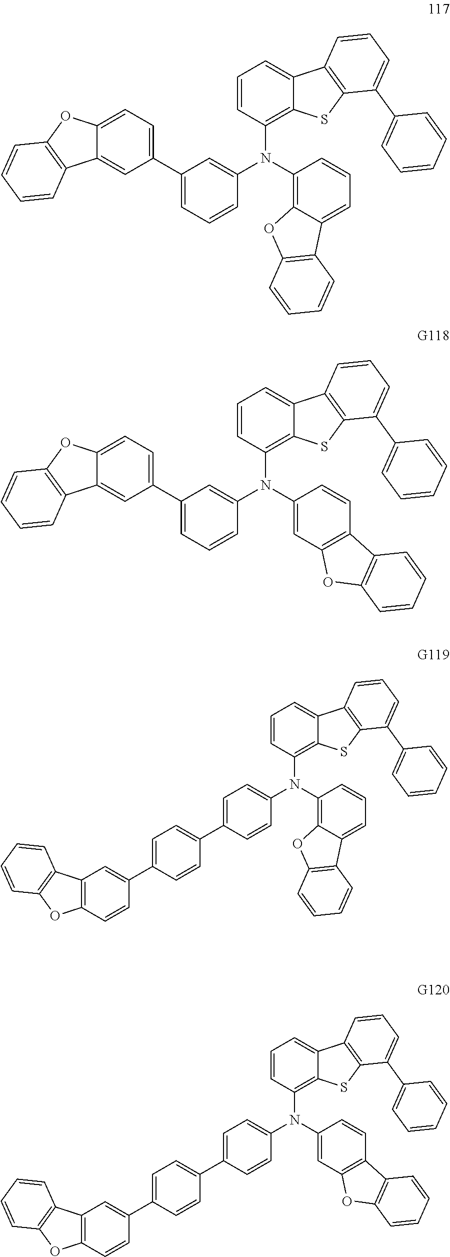 [SMILES: c1ccc(-c2cccc3c2sc2c(N(c4cccc(-c5ccc6oc7ccccc7c6c5)c4)c4cccc5c4oc4ccccc45)cccc23)cc1 ; c1ccc(-c2cccc3c2sc2c(N(c4cccc(-c5ccc6oc7ccccc7c6c5)c4)c4ccc5c(c4)oc4ccccc45)cccc23)cc1 ; c1ccc(-c2cccc3c2sc2c(N(c4ccc(-c5ccc(-c6ccc7oc8ccccc8c7c6)cc5)cc4)c4cccc5c4oc4ccccc45)cccc23)cc1 ; c1ccc(-c2cccc3c2sc2c(N(c4ccc(-c5ccc(-c6ccc7oc8ccccc8c7c6)cc5)cc4)c4ccc5c(c4)oc4ccccc45)cccc23)cc1]

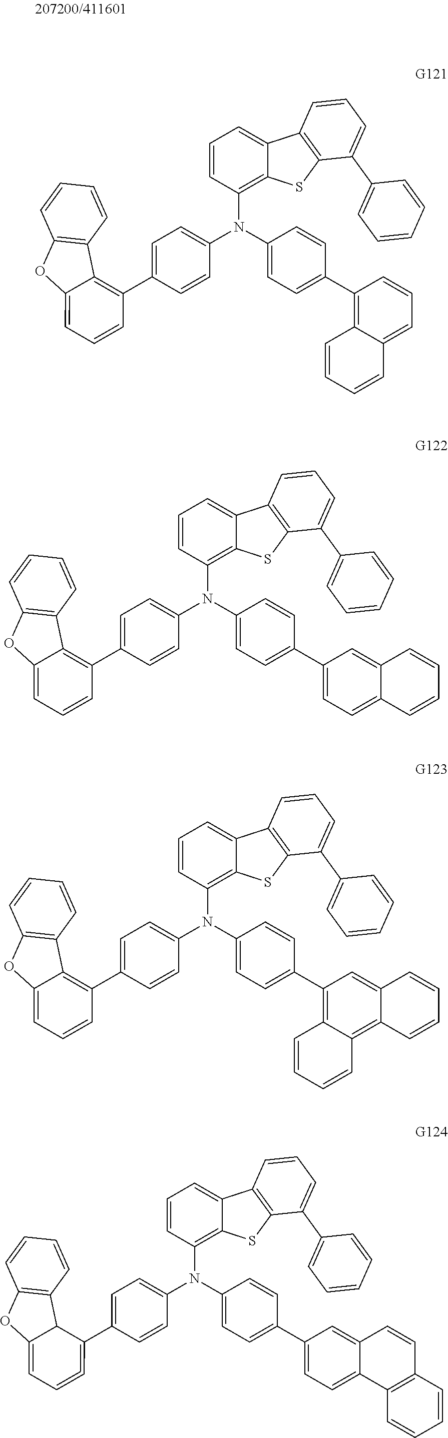 [SMILES: c1ccc(-c2cccc3c2sc2c(N(c4ccc(-c5cccc6ccccc56)cc4)c4ccc(-c5cccc6oc7ccccc7c56)cc4)cccc23)cc1 ; c1ccc(-c2cccc3c2sc2c(N(c4ccc(-c5ccc6ccccc6c5)cc4)c4ccc(-c5cccc6oc7ccccc7c56)cc4)cccc23)cc1 ; c1ccc(-c2cccc3c2sc2c(N(c4ccc(-c5cc6ccccc6c6ccccc56)cc4)c4ccc(-c5cccc6oc7ccccc7c56)cc4)cccc23)cc1 ; C1=CC=C2Oc3ccccc3C2C=1c1ccc(N(c2ccc(-c3ccc4c(ccc5ccccc54)c3)cc2)c2cccc3c2sc2c(-c4ccccc4)cccc23)cc1]

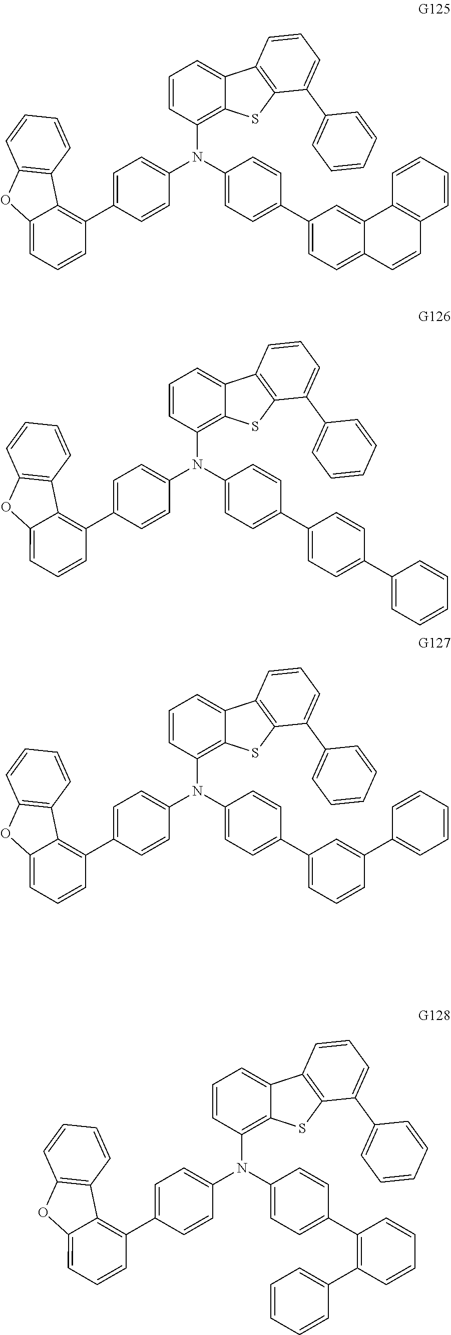 [SMILES: c1ccc(-c2cccc3c2sc2c(N(c4ccc(-c5ccc6ccc7ccccc7c6c5)cc4)c4ccc(-c5cccc6oc7ccccc7c56)cc4)cccc23)cc1 ; c1ccc(-c2ccc(-c3ccc(N(c4ccc(-c5cccc6oc7ccccc7c56)cc4)c4cccc5c4sc4c(-c6ccccc6)cccc45)cc3)cc2)cc1 ; c1ccc(-c2cccc(-c3ccc(N(c4ccc(-c5cccc6oc7ccccc7c56)cc4)c4cccc5c4sc4c(-c6ccccc6)cccc45)cc3)c2)cc1 ; c1ccc(-c2ccccc2-c2ccc(N(c3ccc(-c4cccc5oc6ccccc6c45)cc3)c3cccc4c3sc3c(-c5ccccc5)cccc34)cc2)cc1]

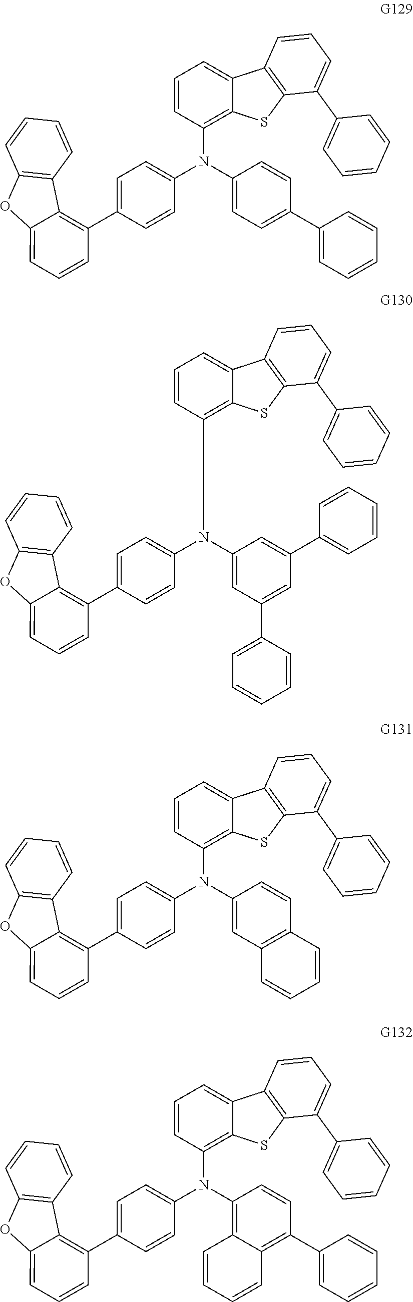 [SMILES: c1ccc(-c2ccc(N(c3ccc(-c4cccc5oc6ccccc6c45)cc3)c3cccc4c3sc3c(-c5ccccc5)cccc34)cc2)cc1 ; c1ccc(-c2cc(-c3ccccc3)cc(N(c3ccc(-c4cccc5oc6ccccc6c45)cc3)c3cccc4c3sc3c(-c5ccccc5)cccc34)c2)cc1 ; c1ccc(-c2cccc3c2sc2c(N(c4ccc(-c5cccc6oc7ccccc7c56)cc4)c4ccc5ccccc5c4)cccc23)cc1 ; c1ccc(-c2ccc(N(c3ccc(-c4cccc5oc6ccccc6c45)cc3)c3cccc4c3sc3c(-c5ccccc5)cccc34)c3ccccc23)cc1]

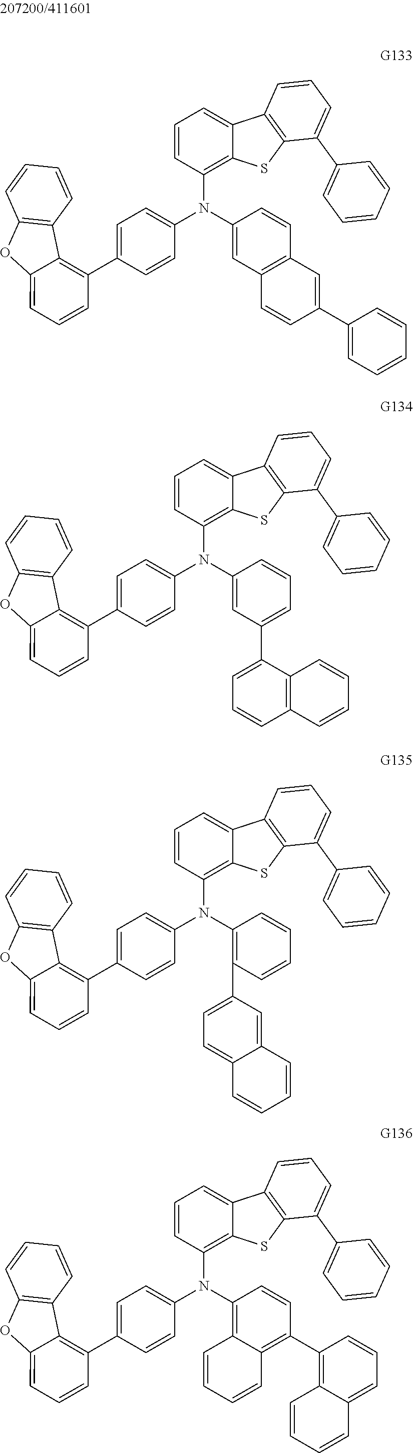 [SMILES: c1ccc(-c2ccc3cc(N(c4ccc(-c5cccc6oc7ccccc7c56)cc4)c4cccc5c4sc4c(-c6ccccc6)cccc45)ccc3c2)cc1 ; c1ccc(-c2cccc3c2sc2c(N(c4ccc(-c5cccc6oc7ccccc7c56)cc4)c4cccc(-c5cccc6ccccc56)c4)cccc23)cc1 ; c1ccc(-c2cccc3c2sc2c(N(c4ccc(-c5cccc6oc7ccccc7c56)cc4)c4ccccc4-c4ccc5ccccc5c4)cccc23)cc1 ; c1ccc(-c2cccc3c2sc2c(N(c4ccc(-c5cccc6oc7ccccc7c56)cc4)c4ccc(-c5cccc6ccccc56)c5ccccc45)cccc23)cc1]

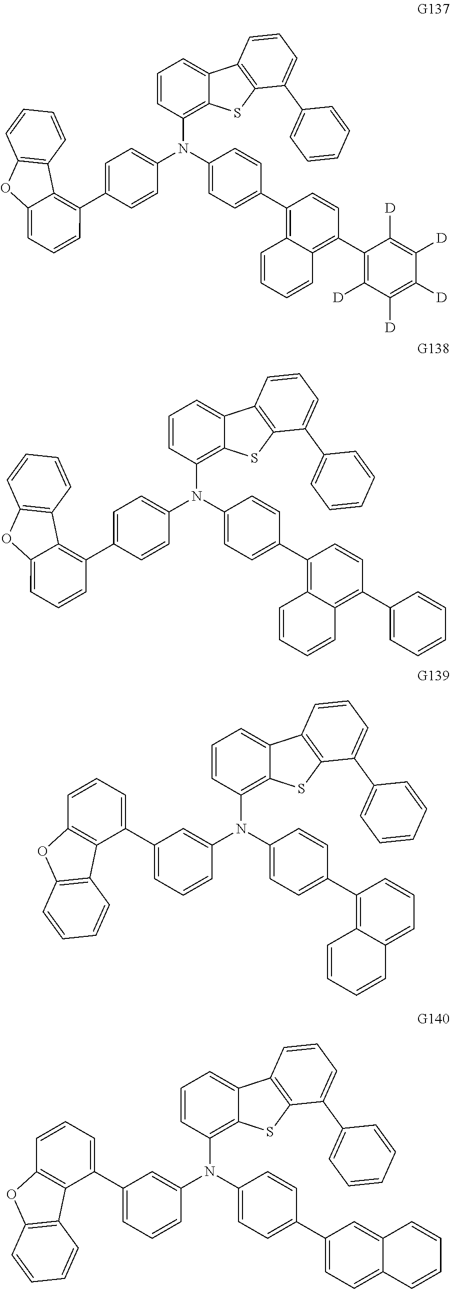 [SMILES: [2H]c1c([2H])c([2H])c(-c2ccc(-c3ccc(N(c4ccc(-c5cccc6oc7ccccc7c56)cc4)c4cccc5c4sc4c(-c6ccccc6)cccc45)cc3)c3ccccc23)c([2H])c1[2H] ; c1ccc(-c2ccc(-c3ccc(N(c4ccc(-c5cccc6oc7ccccc7c56)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4cccc(-c5cccc6oc7ccccc7c56)c4)cccc23)cc1 ; c1ccc(-c2cccc3c2sc2c(N(c4ccc(-c5ccc6ccccc6c5)cc4)c4cccc(-c5cccc6oc7ccccc7c56)c4)cccc23)cc1]

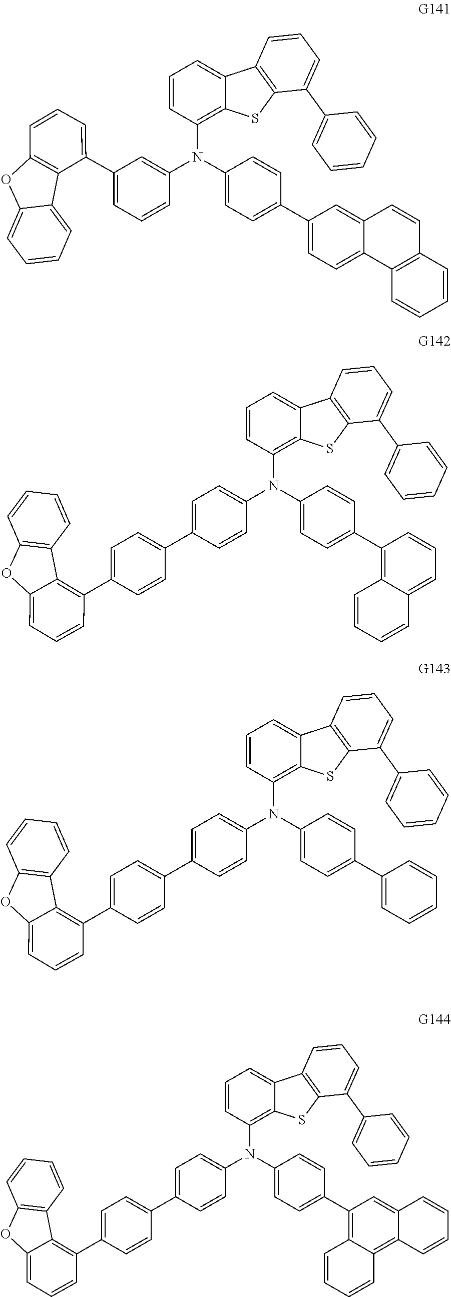 [SMILES: c1ccc(-c2cccc3c2sc2c(N(c4ccc(-c5ccc6c(ccc7ccccc76)c5)cc4)c4cccc(-c5cccc6oc7ccccc7c56)c4)cccc23)cc1 ; c1ccc(-c2cccc3c2sc2c(N(c4ccc(-c5ccc(-c6cccc7oc8ccccc8c67)cc5)cc4)c4ccc(-c5cccc6ccccc56)cc4)cccc23)cc1 ; c1ccc(-c2ccc(N(c3ccc(-c4ccc(-c5cccc6oc7ccccc7c56)cc4)cc3)c3cccc4c3sc3c(-c5ccccc5)cccc34)cc2)cc1 ; c1ccc(-c2cccc3c2sc2c(N(c4ccc(-c5ccc(-c6cccc7oc8ccccc8c67)cc5)cc4)c4ccc(-c5cc6ccccc6c6ccccc56)cc4)cccc23)cc1]

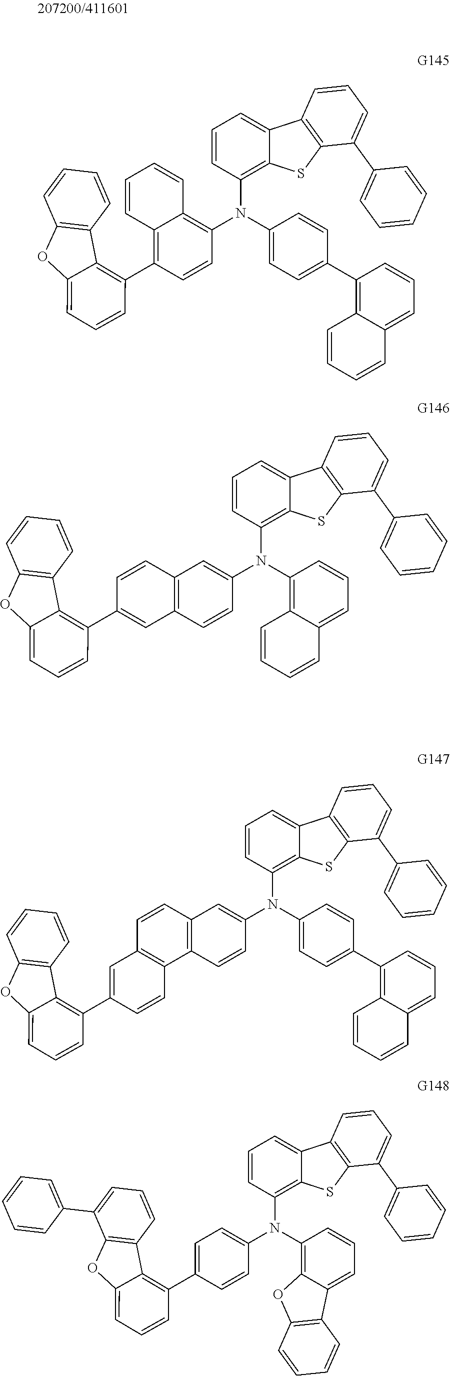 [SMILES: c1ccc(-c2cccc3c2sc2c(N(c4ccc(-c5cccc6ccccc56)cc4)c4ccc(-c5cccc6oc7ccccc7c56)c5ccccc45)cccc23)cc1 ; c1ccc(-c2cccc3c2sc2c(N(c4ccc5cc(-c6cccc7oc8ccccc8c67)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7cccc8oc9ccccc9c78)ccc65)c4)cccc23)cc1 ; c1ccc(-c2cccc3c2oc2cccc(-c4ccc(N(c5cccc6c5oc5ccccc56)c5cccc6c5sc5c(-c7ccccc7)cccc56)cc4)c23)cc1]

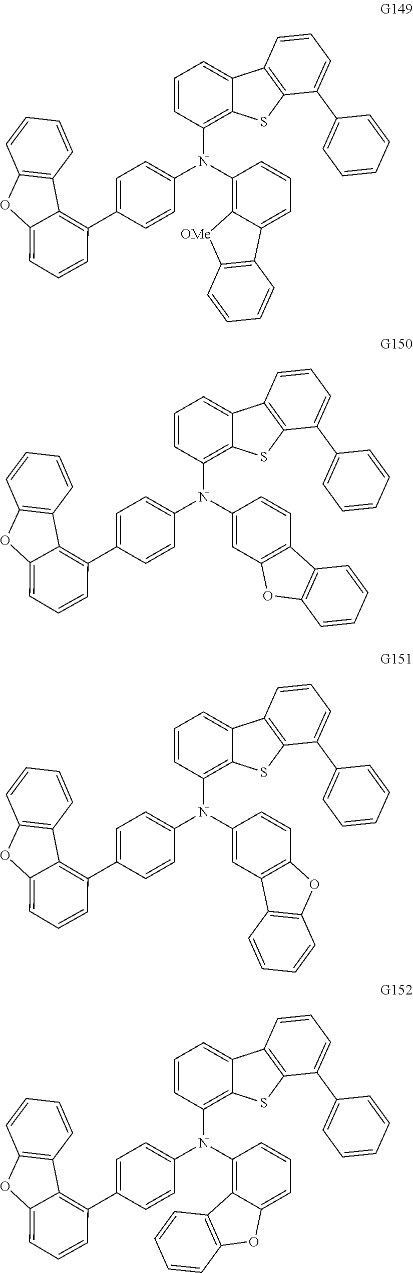

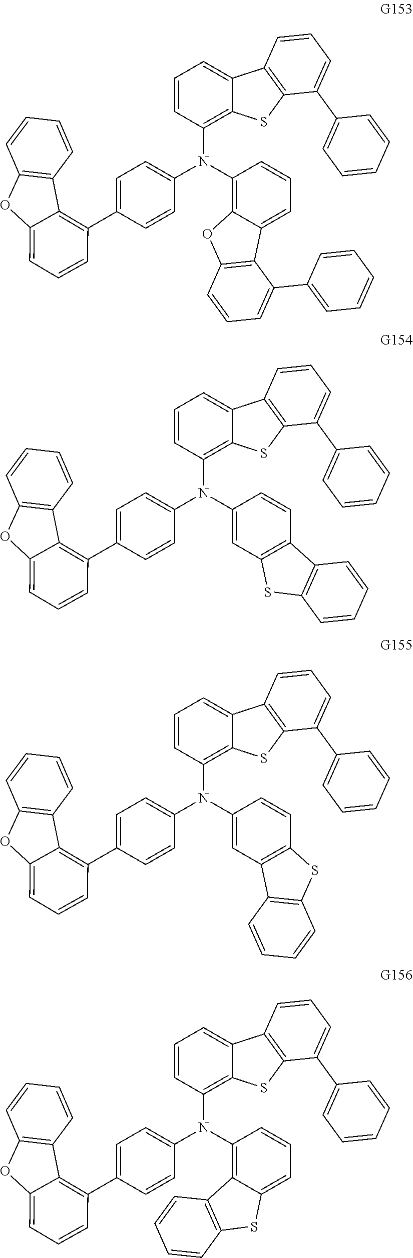

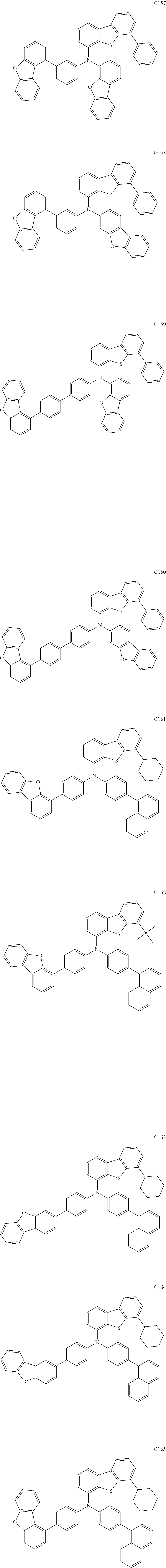

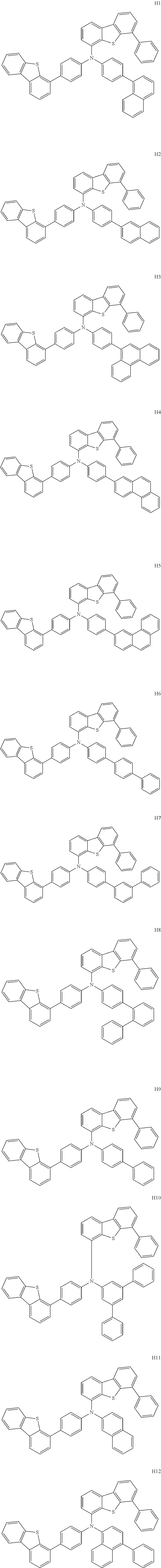

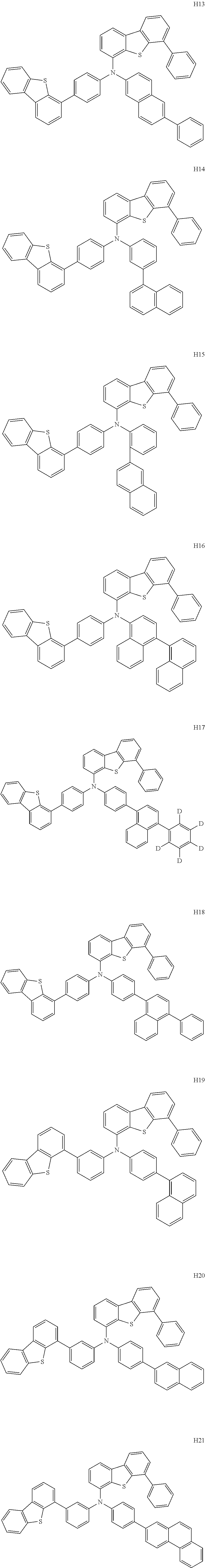

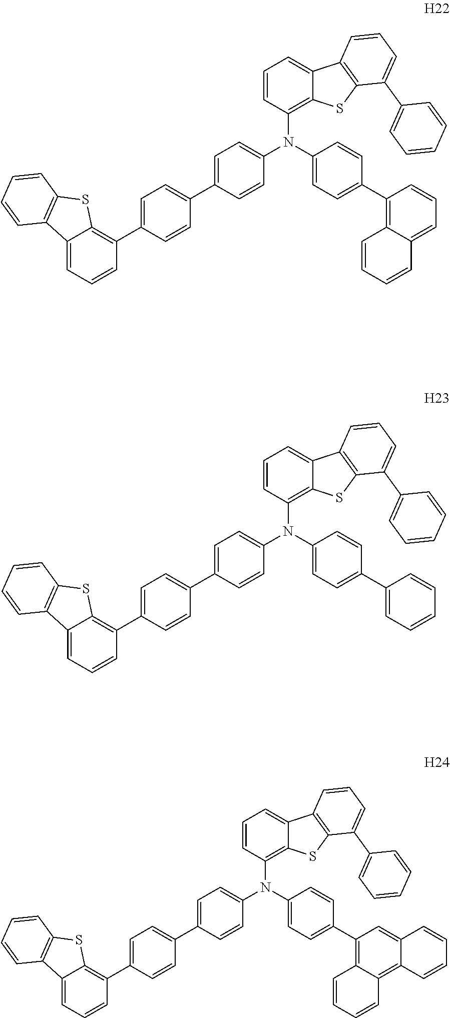 [SMILES: c1ccc(-c2cccc3c2sc2c(N(c4ccc(-c5ccc(-c6cccc7c6sc6ccccc67)cc5)cc4)c4ccc(-c5cccc6ccccc56)cc4)cccc23)cc1 ; c1ccc(-c2ccc(N(c3ccc(-c4ccc(-c5cccc6c5sc5ccccc56)cc4)cc3)c3cccc4c3sc3c(-c5ccccc5)cccc34)cc2)cc1 ; c1ccc(-c2cccc3c2sc2c(N(c4ccc(-c5ccc(-c6cccc7c6sc6ccccc67)cc5)cc4)c4ccc(-c5cc6ccccc6c6ccccc56)cc4)cccc23)cc1]

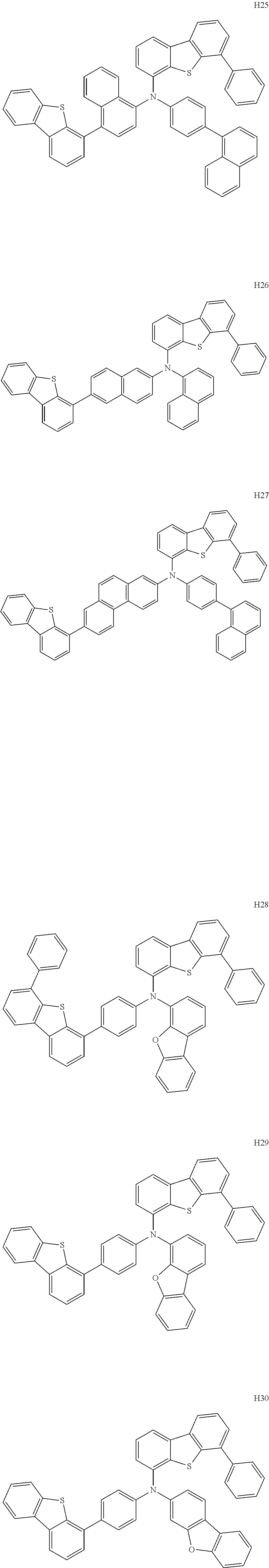

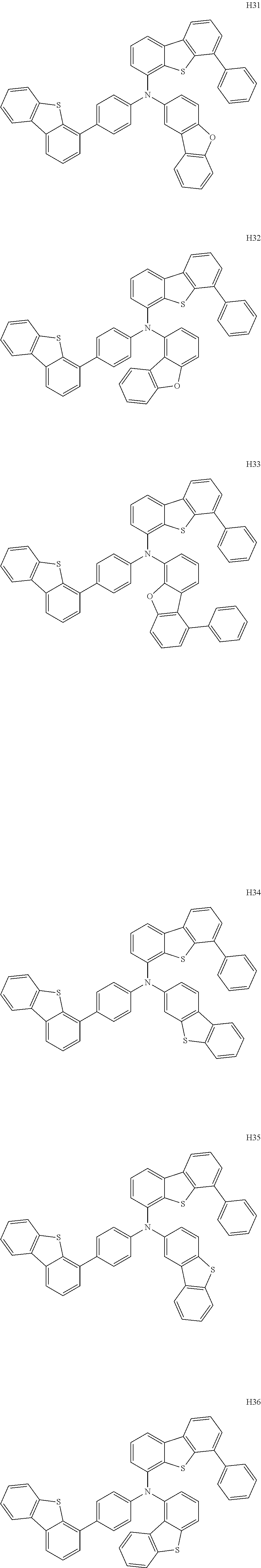

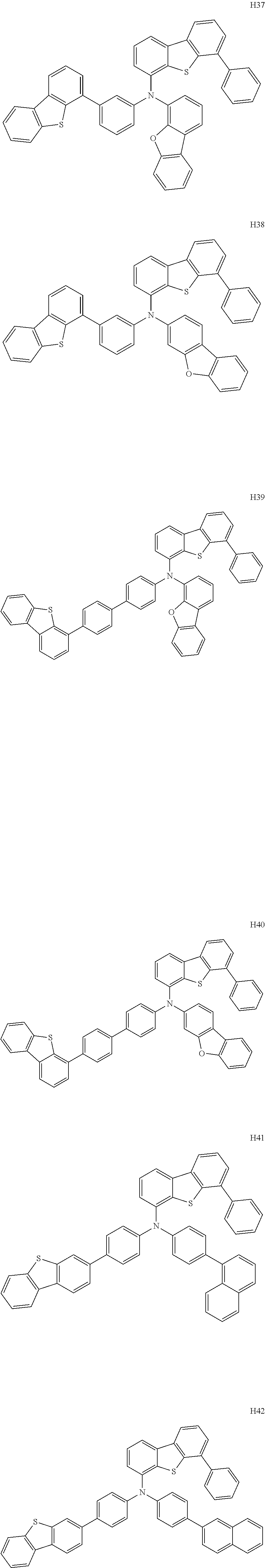

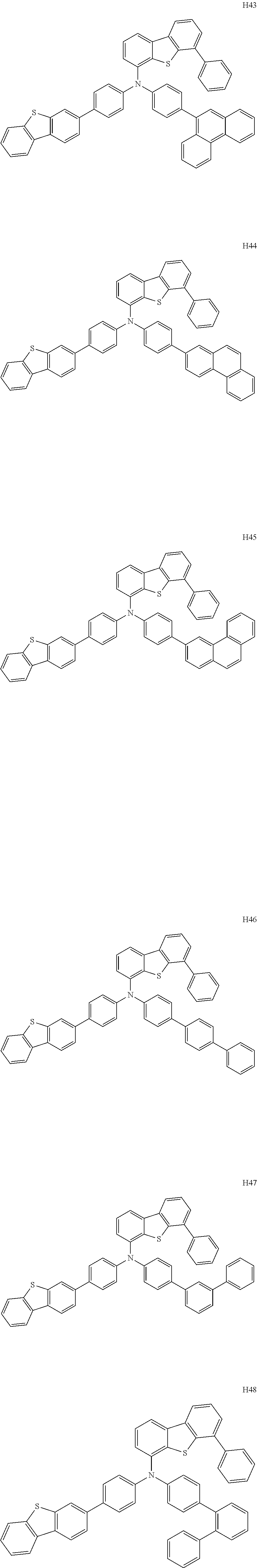

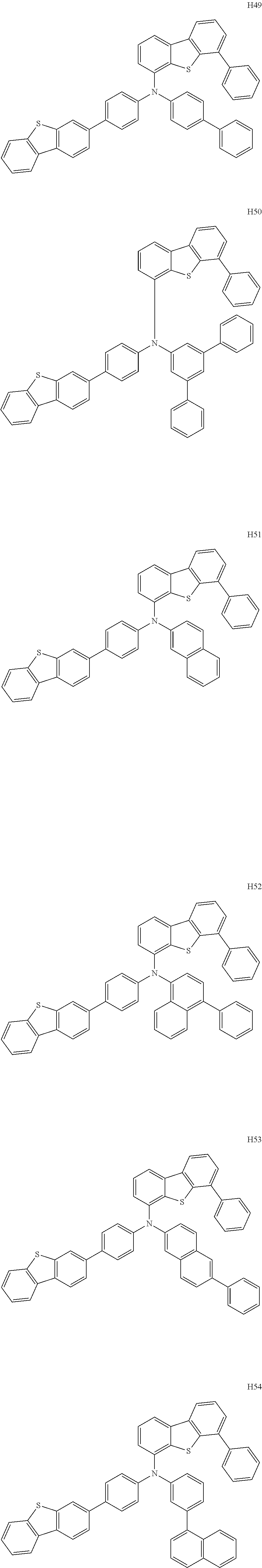

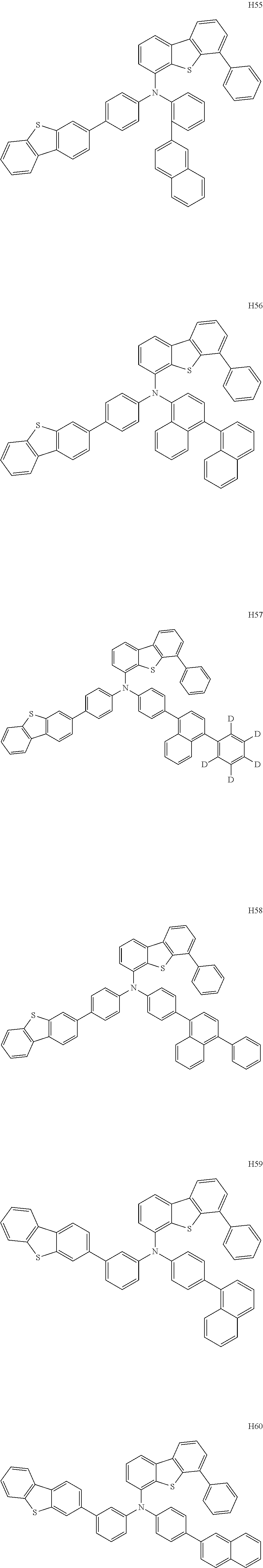

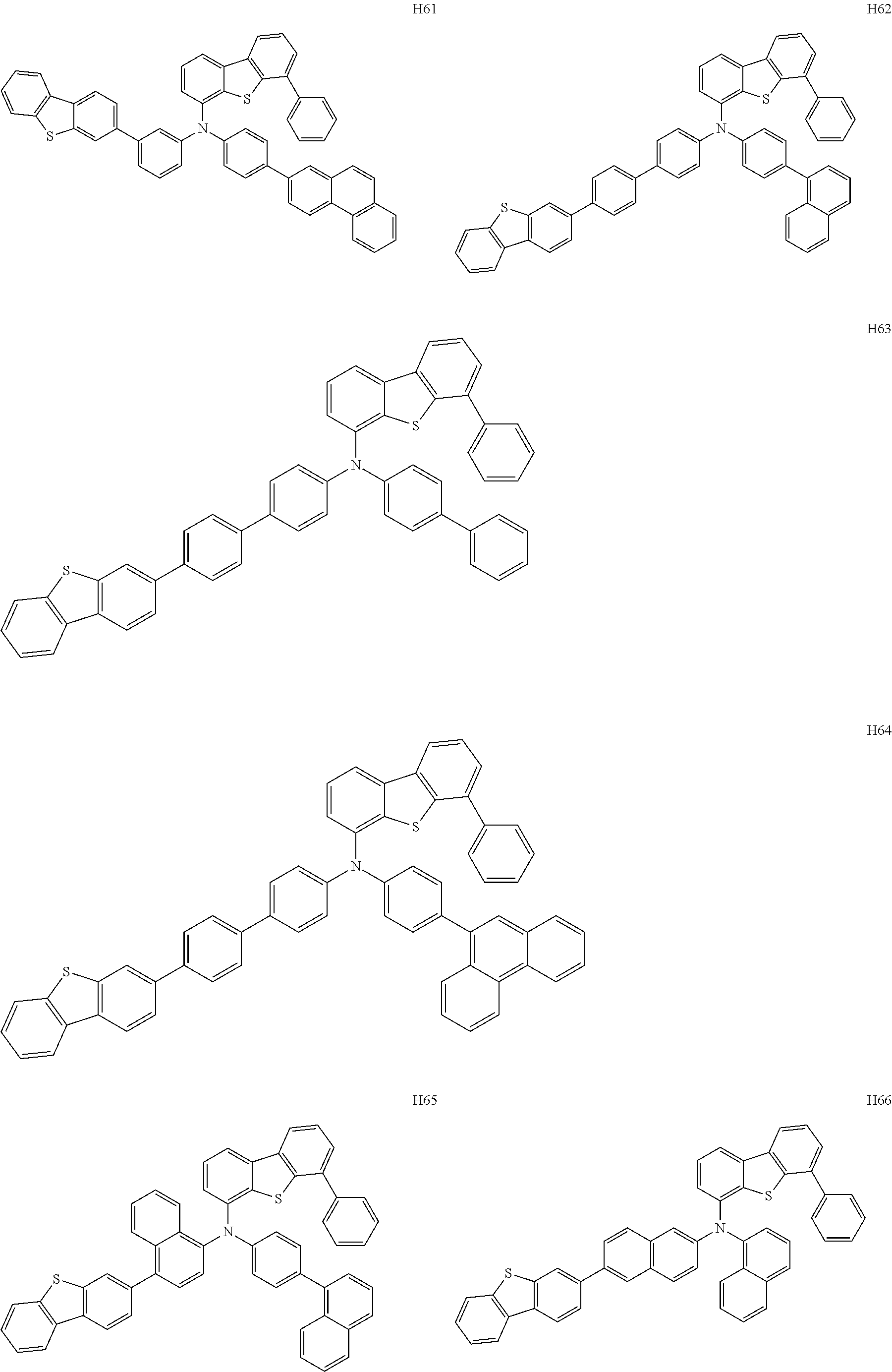

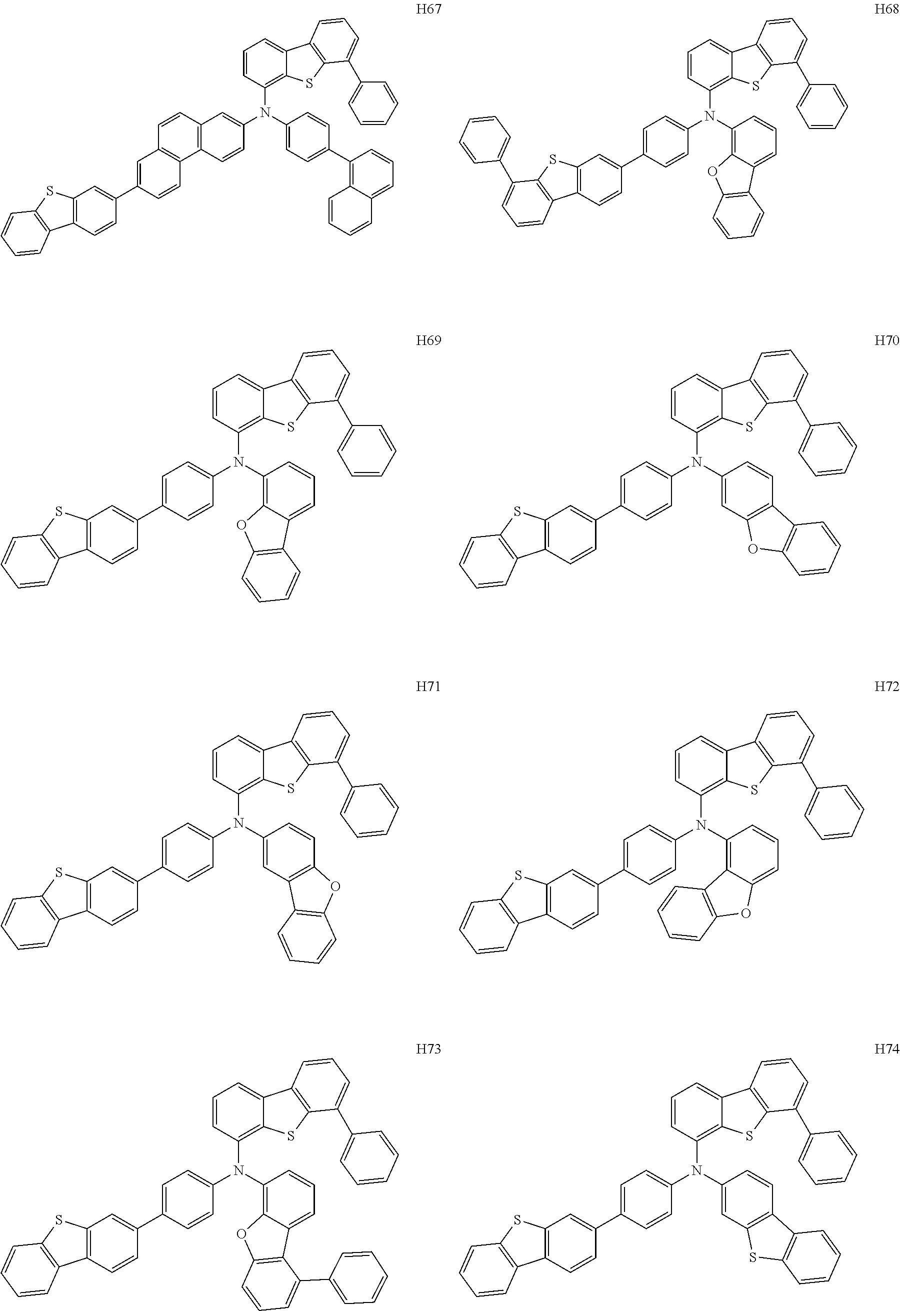

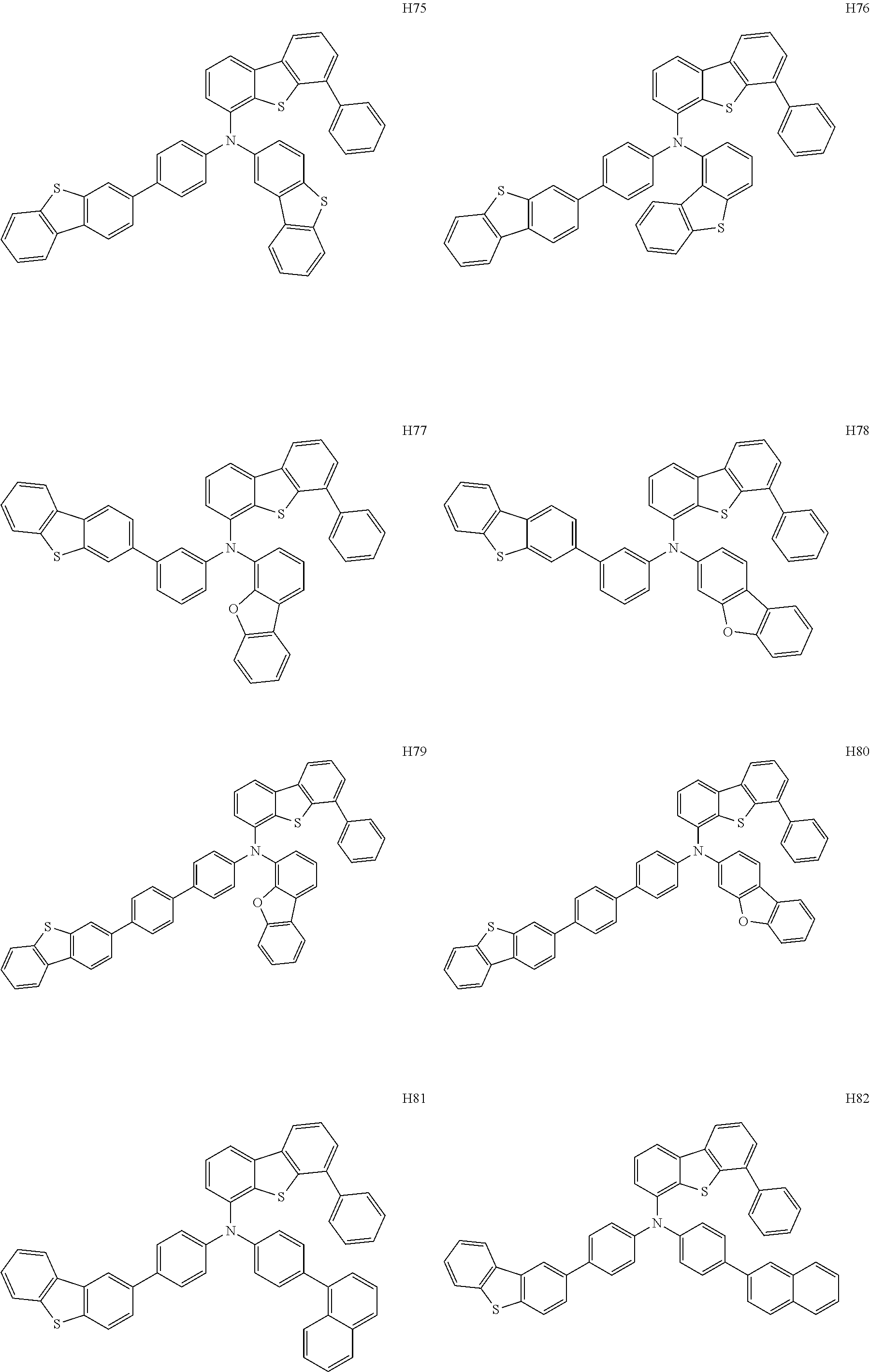

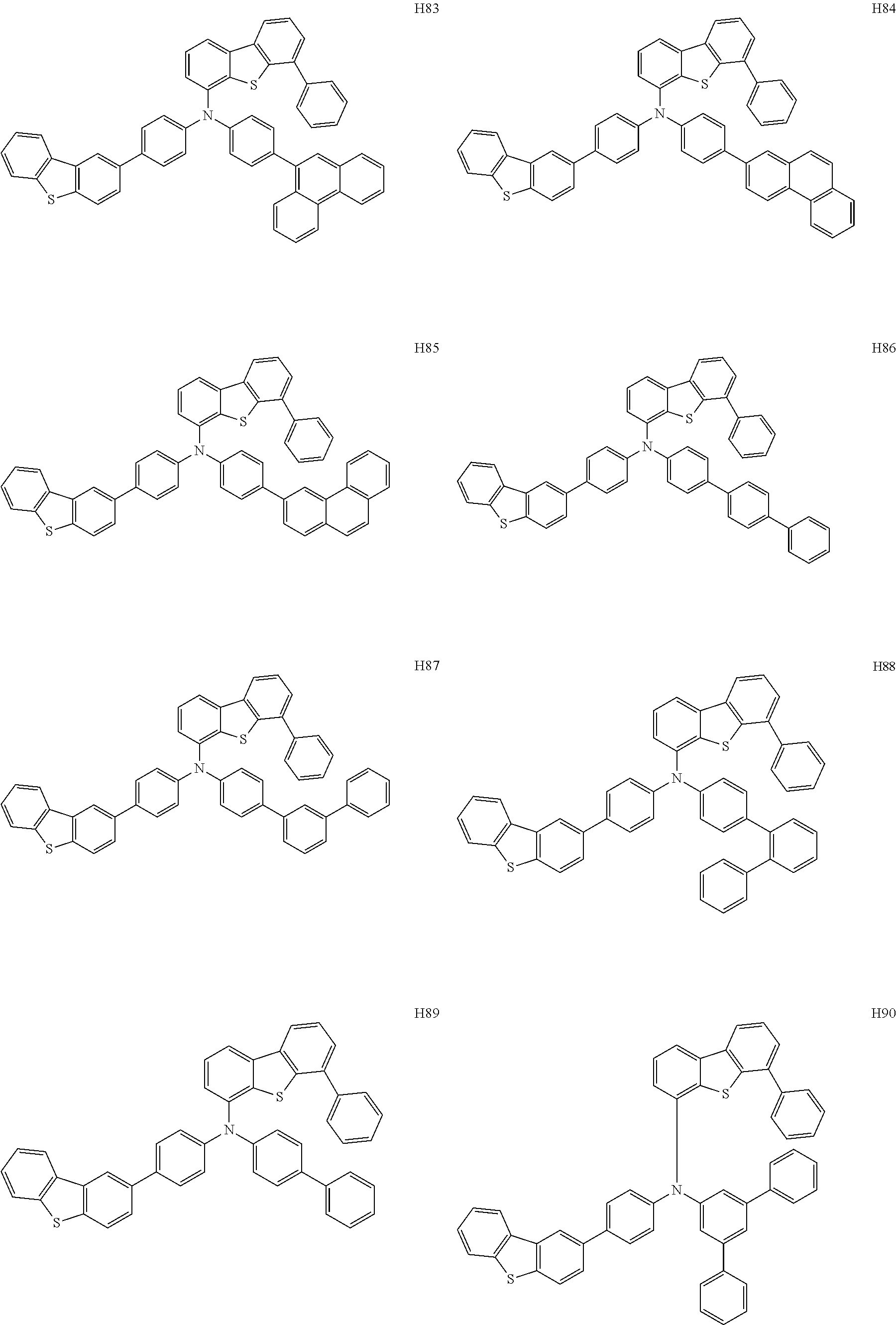

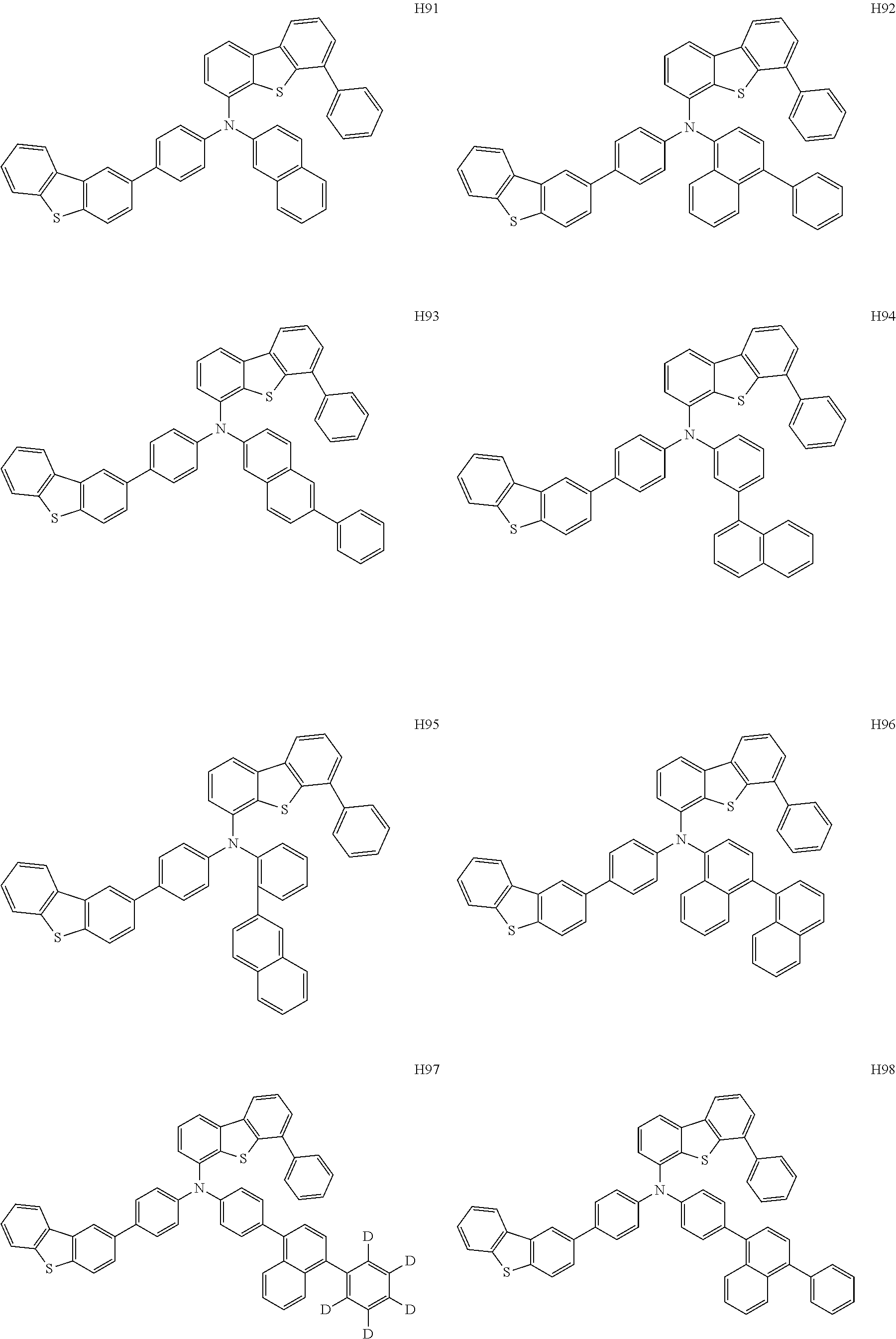

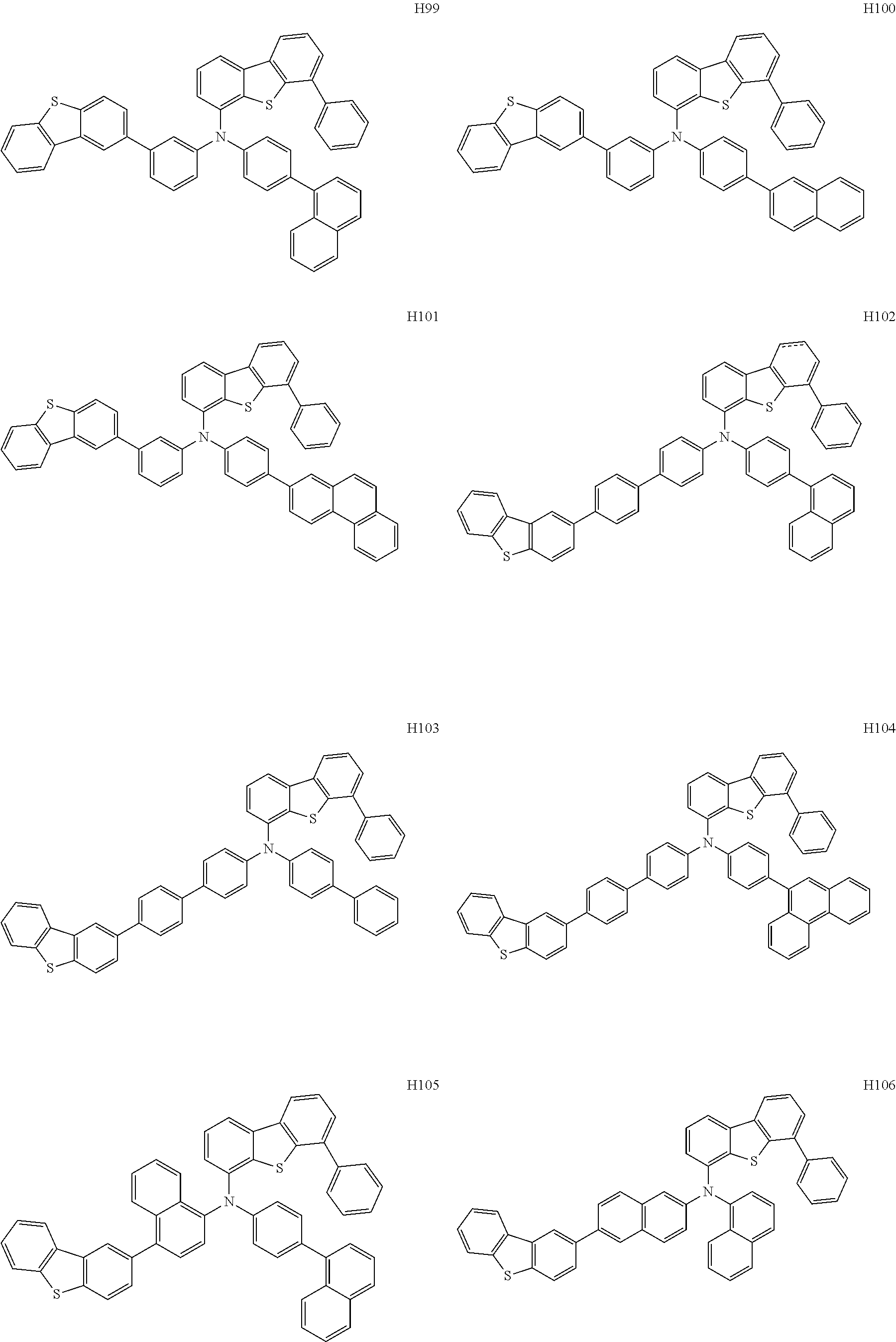

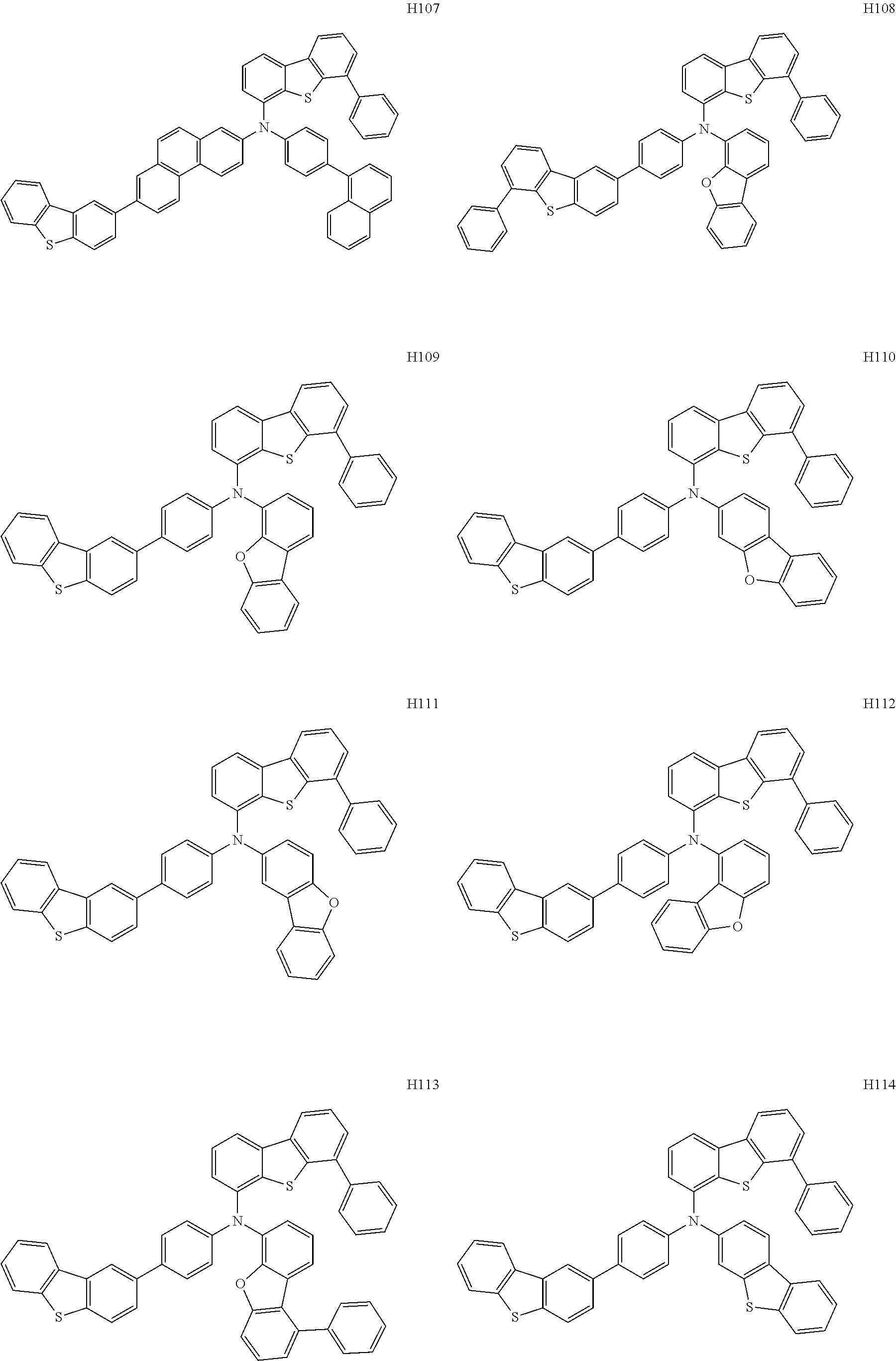

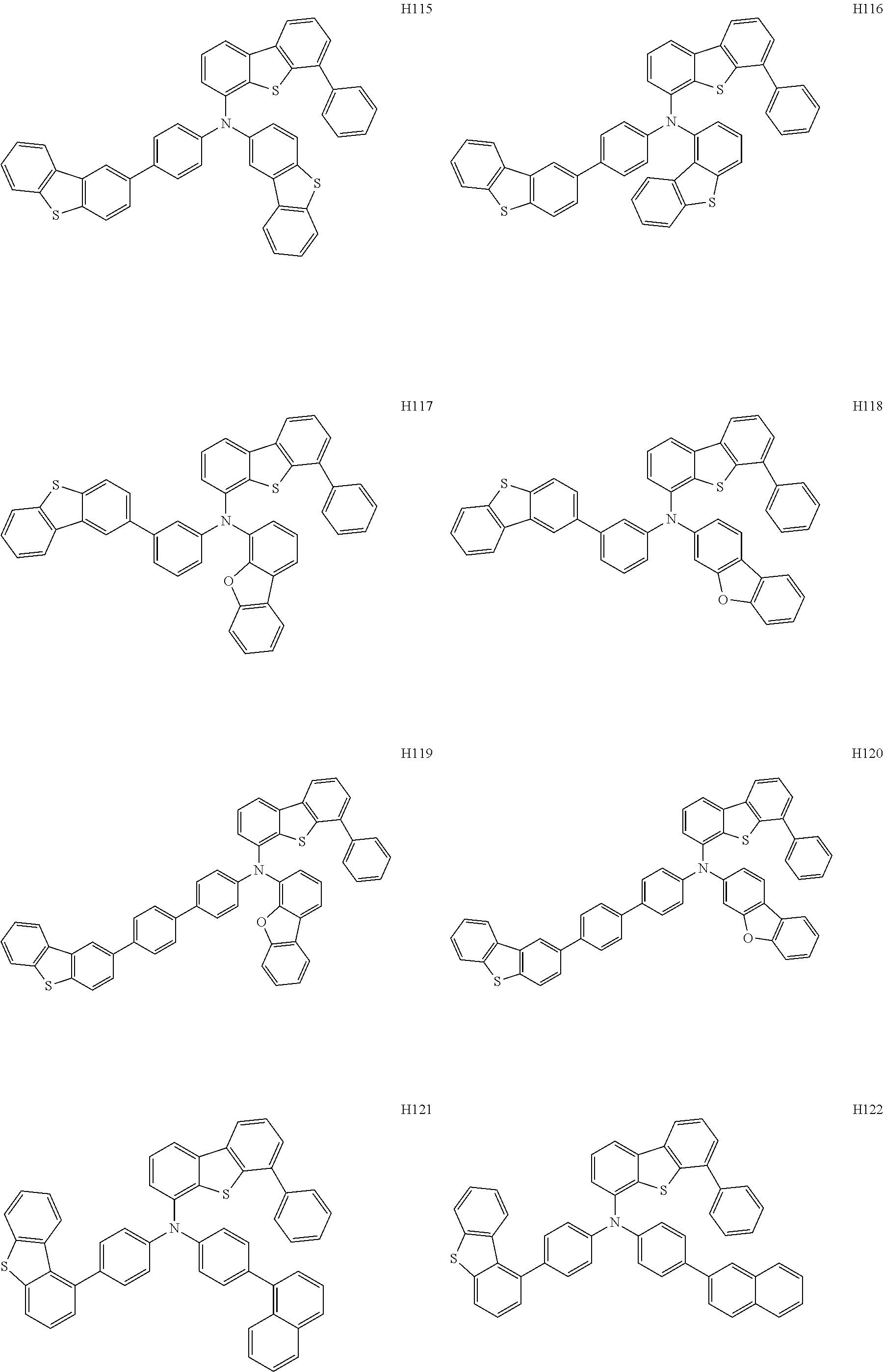

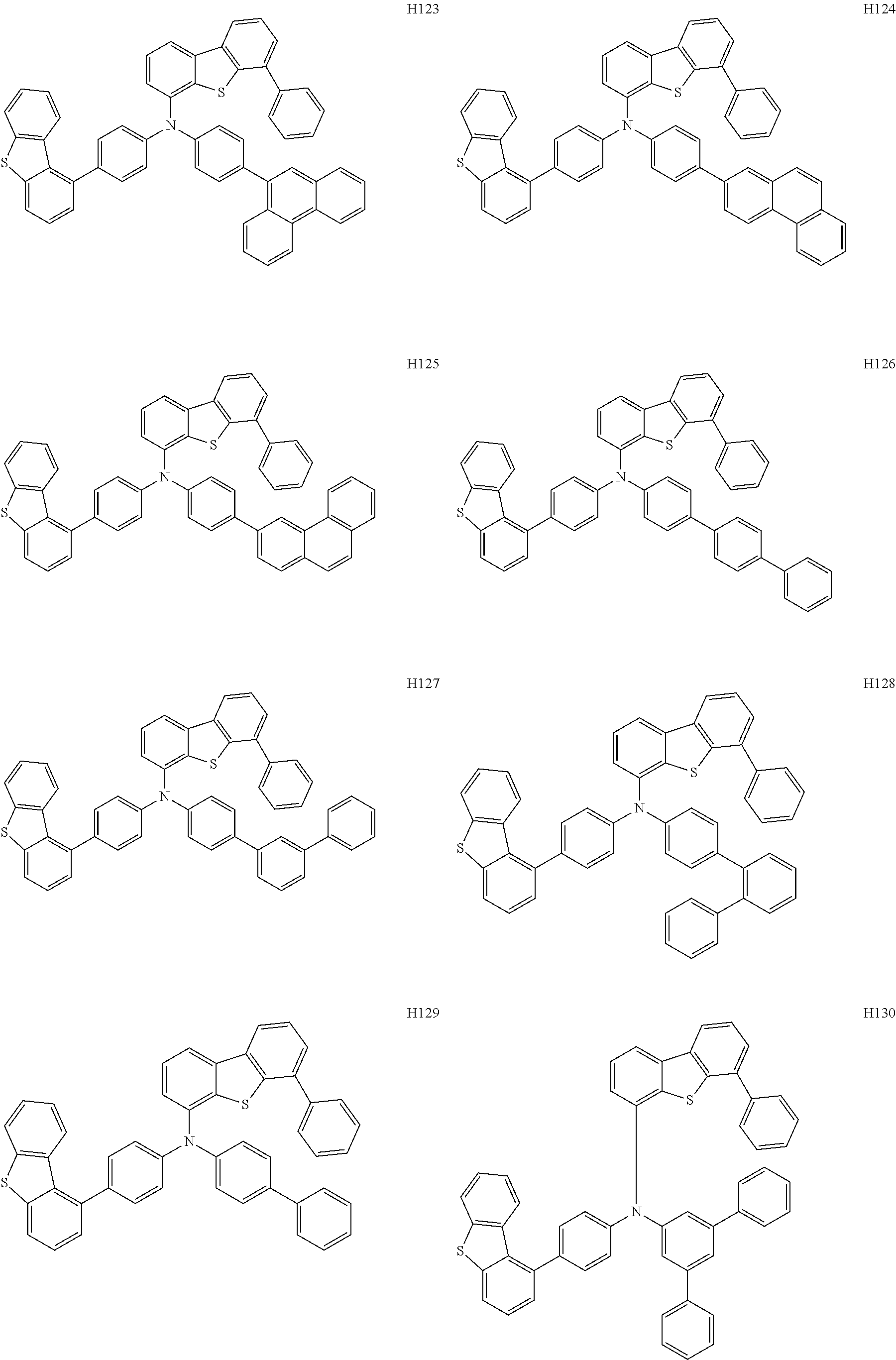

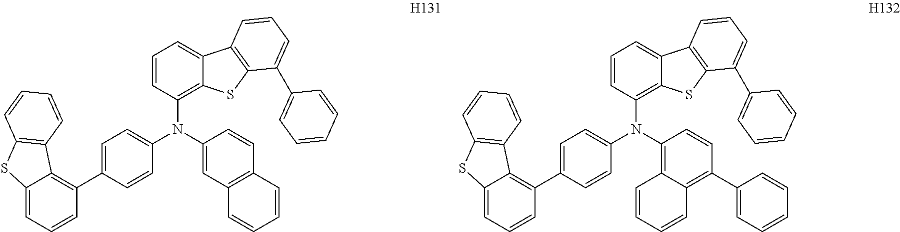 [SMILES: c1ccc(-c2cccc3c2sc2c(N(c4ccc(-c5cccc6sc7ccccc7c56)cc4)c4ccc5ccccc5c4)cccc23)cc1 ; c1ccc(-c2ccc(N(c3ccc(-c4cccc5sc6ccccc6c45)cc3)c3cccc4c3sc3c(-c5ccccc5)cccc34)c3ccccc23)cc1]

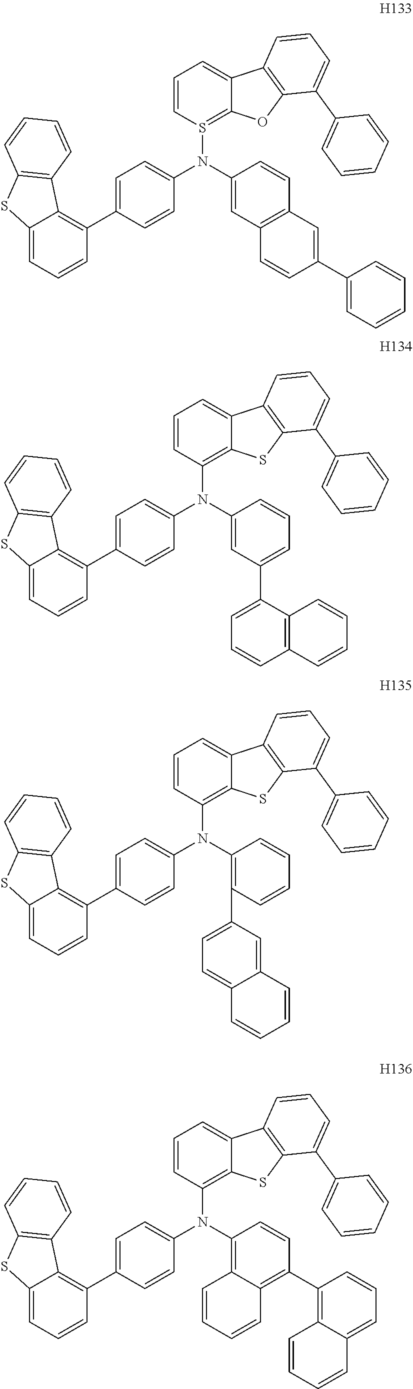

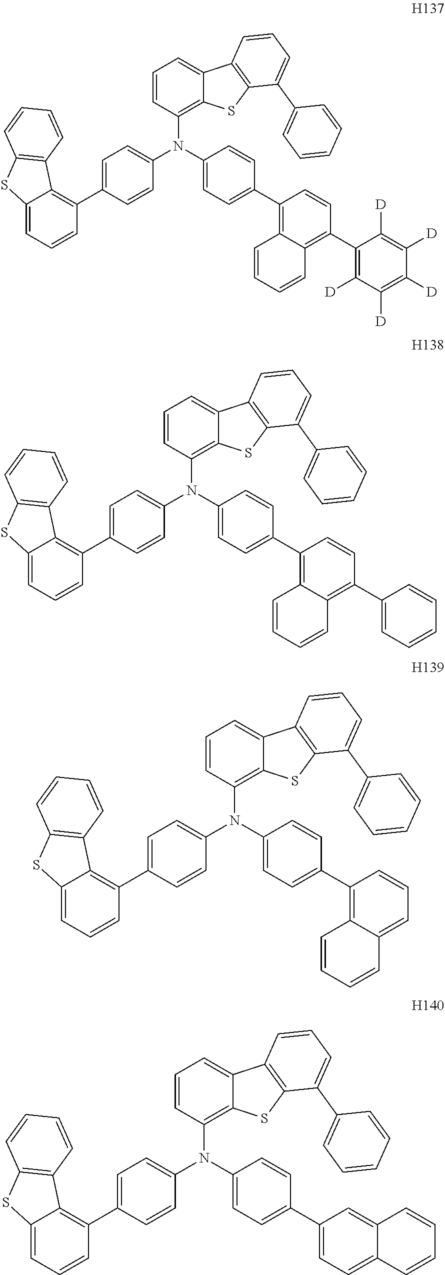 [SMILES: [2H]c1c([2H])c([2H])c(-c2ccc(-c3ccc(N(c4ccc(-c5cccc6sc7ccccc7c56)cc4)c4cccc5c4sc4c(-c6ccccc6)cccc45)cc3)c3ccccc23)c([2H])c1[2H] ; c1ccc(-c2ccc(-c3ccc(N(c4ccc(-c5cccc6sc7ccccc7c56)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4ccc(-c5cccc6sc7ccccc7c56)cc4)cccc23)cc1 ; c1ccc(-c2cccc3c2sc2c(N(c4ccc(-c5ccc6ccccc6c5)cc4)c4ccc(-c5cccc6sc7ccccc7c56)cc4)cccc23)cc1]

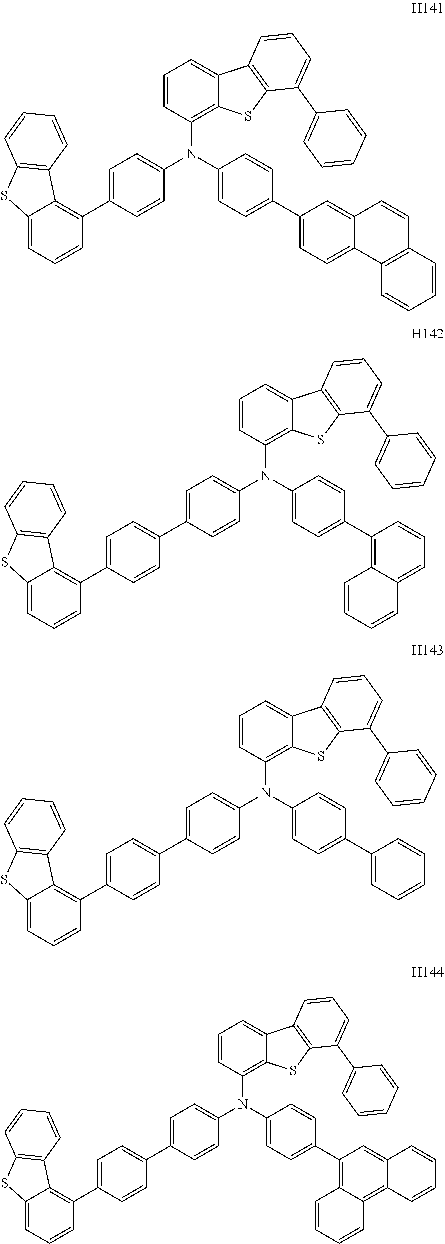 [SMILES: c1ccc(-c2cccc3c2sc2c(N(c4ccc(-c5ccc6c(ccc7ccccc76)c5)cc4)c4ccc(-c5cccc6sc7ccccc7c56)cc4)cccc23)cc1 ; c1ccc(-c2cccc3c2sc2c(N(c4ccc(-c5ccc(-c6cccc7sc8ccccc8c67)cc5)cc4)c4ccc(-c5cccc6ccccc56)cc4)cccc23)cc1 ; c1ccc(-c2ccc(N(c3ccc(-c4ccc(-c5cccc6sc7ccccc7c56)cc4)cc3)c3cccc4c3sc3c(-c5ccccc5)cccc34)cc2)cc1 ; c1ccc(-c2cccc3c2sc2c(N(c4ccc(-c5ccc(-c6cccc7sc8ccccc8c67)cc5)cc4)c4ccc(-c5cc6ccccc6c6ccccc56)cc4)cccc23)cc1]

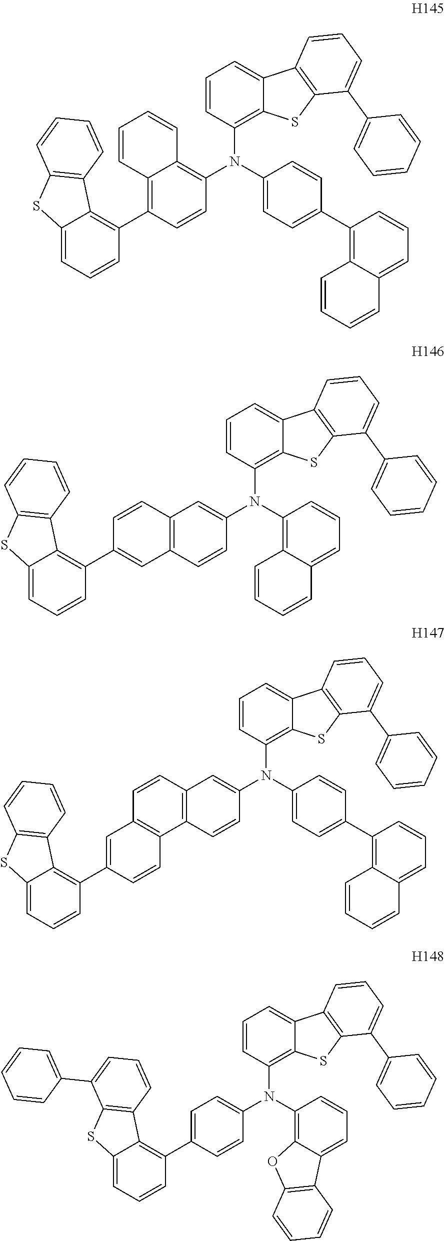 [SMILES: c1ccc(-c2cccc3c2sc2c(N(c4ccc(-c5cccc6ccccc56)cc4)c4ccc(-c5cccc6sc7ccccc7c56)c5ccccc45)cccc23)cc1 ; c1ccc(-c2cccc3c2sc2c(N(c4ccc5cc(-c6cccc7sc8ccccc8c67)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7cccc8sc9ccccc9c78)ccc65)c4)cccc23)cc1 ; c1ccc(-c2cccc3c2sc2c(N(c4ccc(-c5cccc6sc7c(-c8ccccc8)cccc7c56)cc4)c4cccc5c4oc4ccccc45)cccc23)cc1]

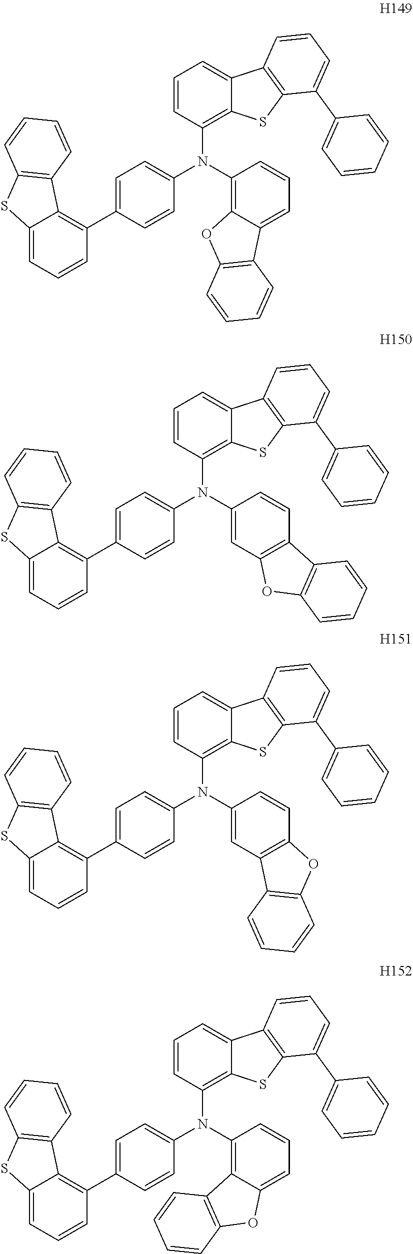

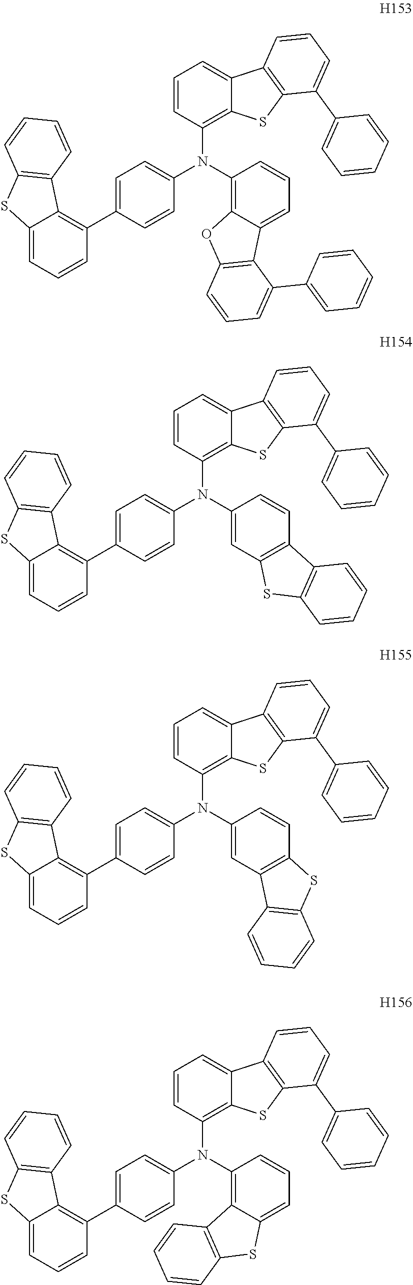 [SMILES: c1ccc(-c2cccc3c2sc2c(N(c4ccc(-c5cccc6sc7ccccc7c56)cc4)c4cccc5c4oc4cccc(-c6ccccc6)c45)cccc23)cc1 ; c1ccc(-c2cccc3c2sc2c(N(c4ccc(-c5cccc6sc7ccccc7c56)cc4)c4ccc5c(c4)sc4ccccc45)cccc23)cc1 ; c1ccc(-c2cccc3c2sc2c(N(c4ccc(-c5cccc6sc7ccccc7c56)cc4)c4ccc5sc6ccccc6c5c4)cccc23)cc1 ; c1ccc(-c2cccc3c2sc2c(N(c4ccc(-c5cccc6sc7ccccc7c56)cc4)c4cccc5sc6ccccc6c45)cccc23)cc1]

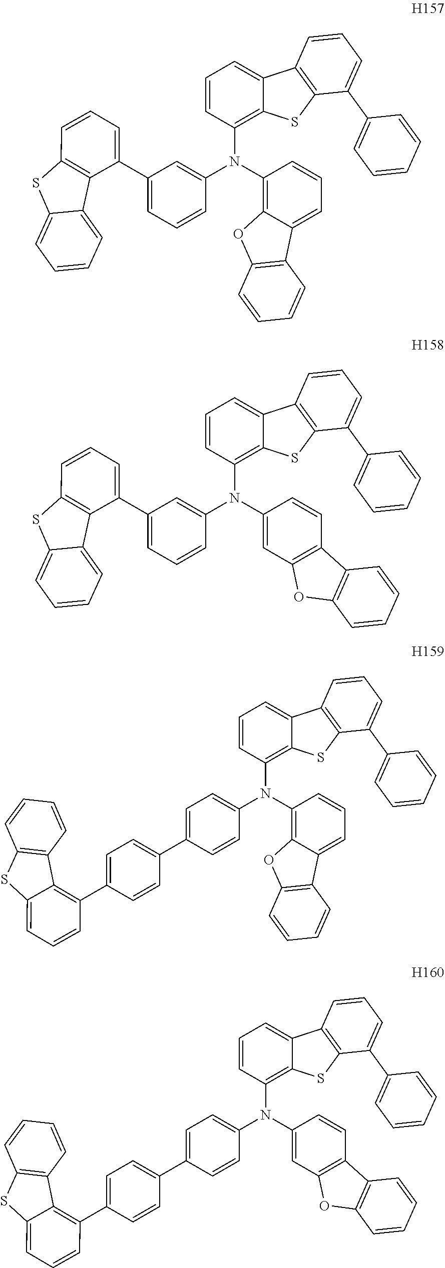 [SMILES: c1ccc(-c2cccc3c2sc2c(N(c4cccc(-c5cccc6sc7ccccc7c56)c4)c4cccc5c4oc4ccccc45)cccc23)cc1 ; c1ccc(-c2cccc3c2sc2c(N(c4cccc(-c5cccc6sc7ccccc7c56)c4)c4ccc5c(c4)oc4ccccc45)cccc23)cc1 ; c1ccc(-c2cccc3c2sc2c(N(c4ccc(-c5ccc(-c6cccc7sc8ccccc8c67)cc5)cc4)c4cccc5c4oc4ccccc45)cccc23)cc1 ; c1ccc(-c2cccc3c2sc2c(N(c4ccc(-c5ccc(-c6cccc7sc8ccccc8c67)cc5)cc4)c4ccc5c(c4)oc4ccccc45)cccc23)cc1]

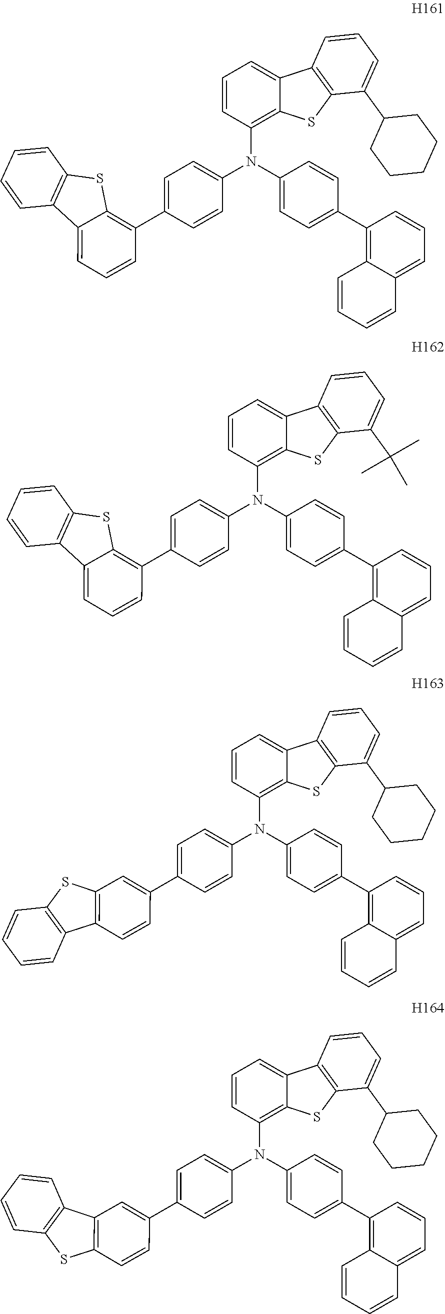

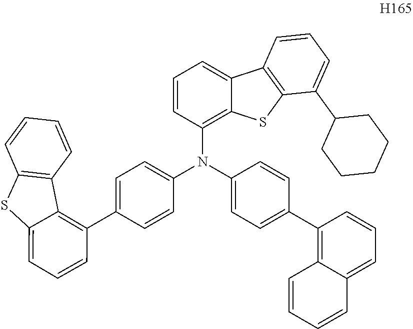 [SMILES: c1ccc2c(-c3ccc(N(c4ccc(-c5cccc6sc7ccccc7c56)cc4)c4cccc5c4sc4c(C6CCCCC6)cccc45)cc3)cccc2c1]

13. The luminescence device of claim 1, wherein the amine compound represented by Formula 1 is any one selected from among compounds represented in

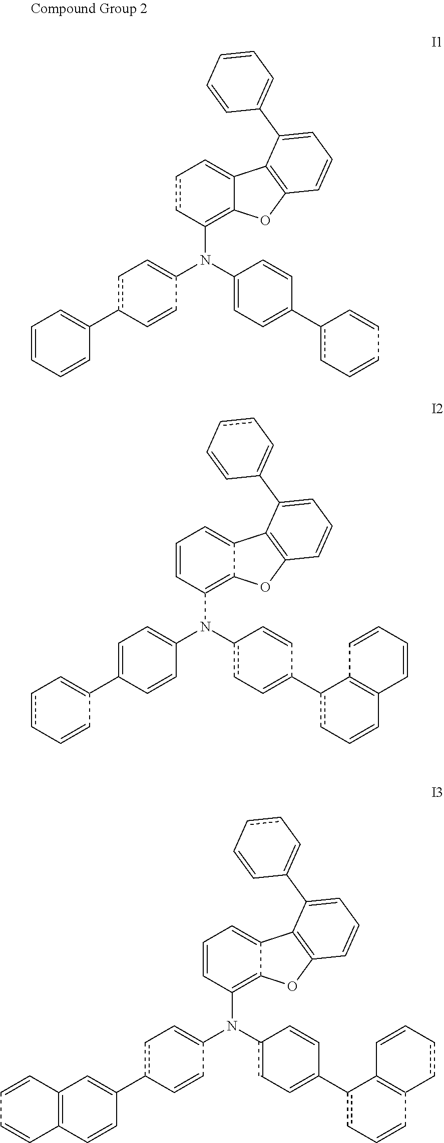

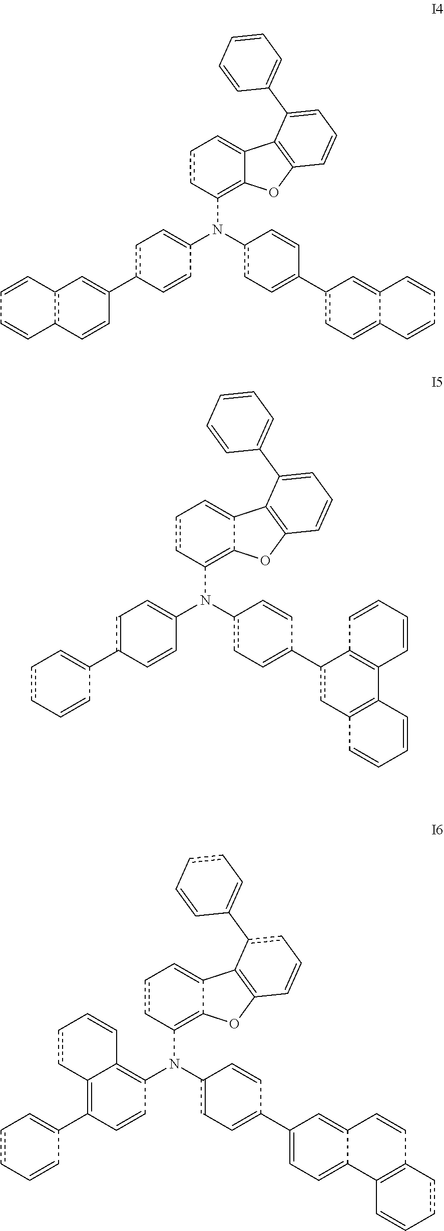

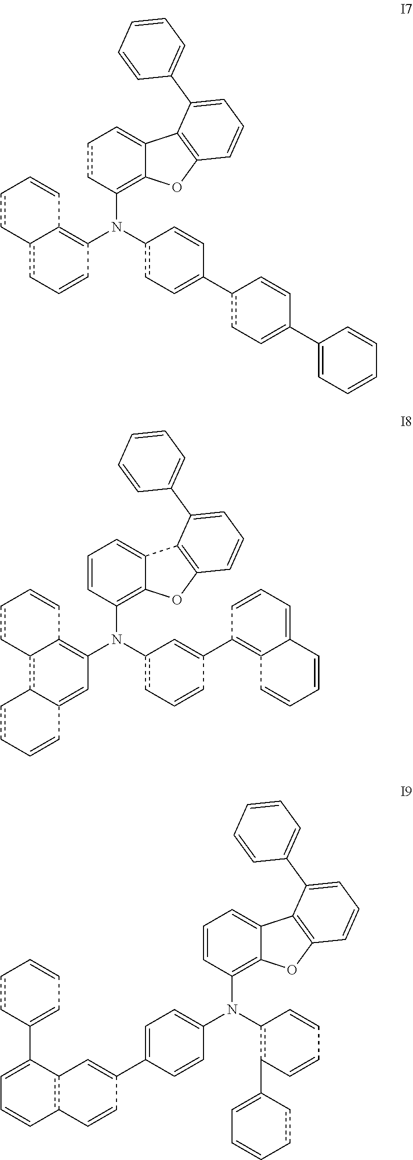

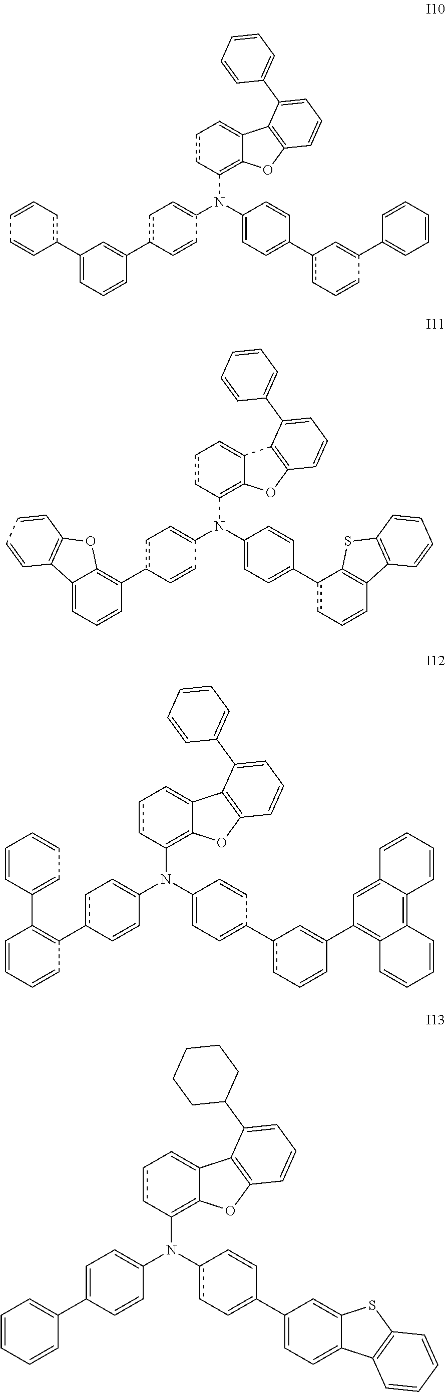

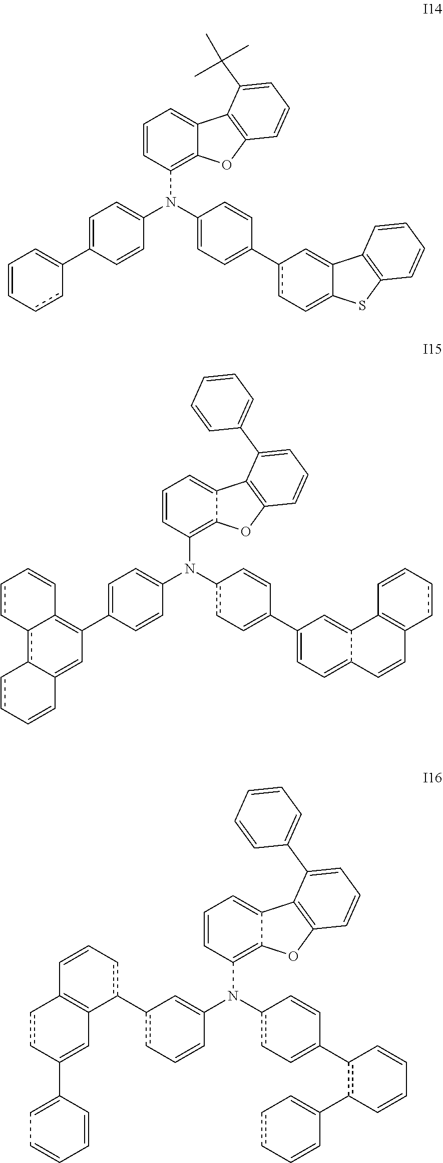

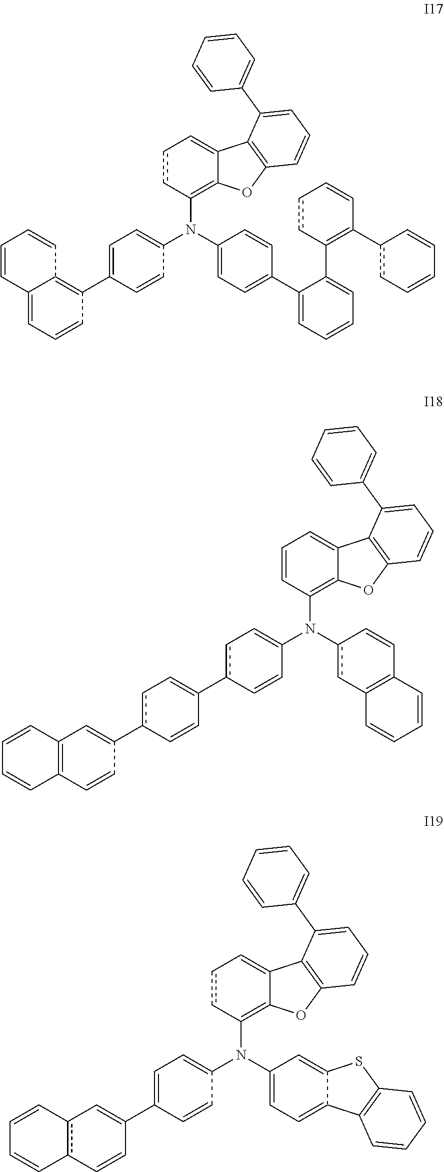

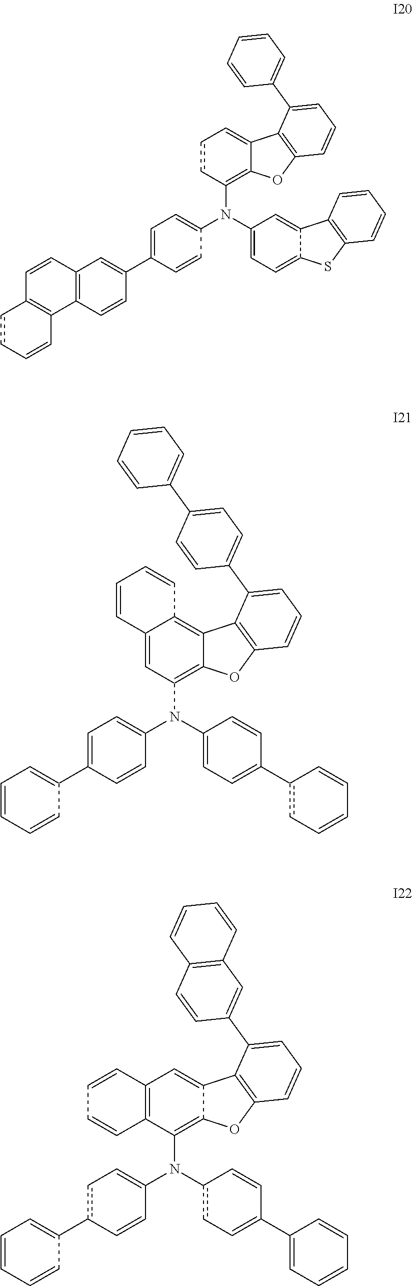

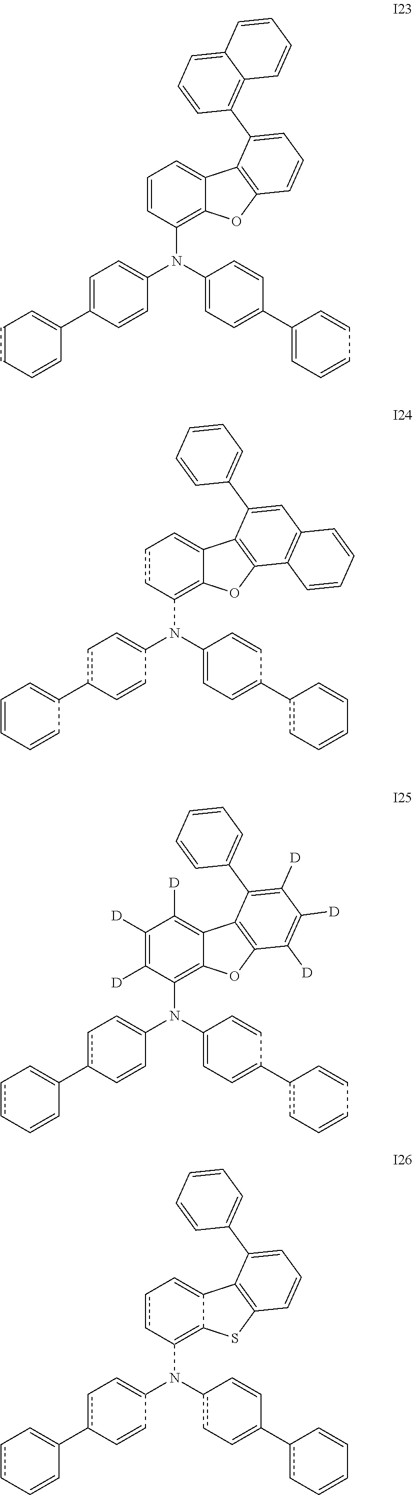

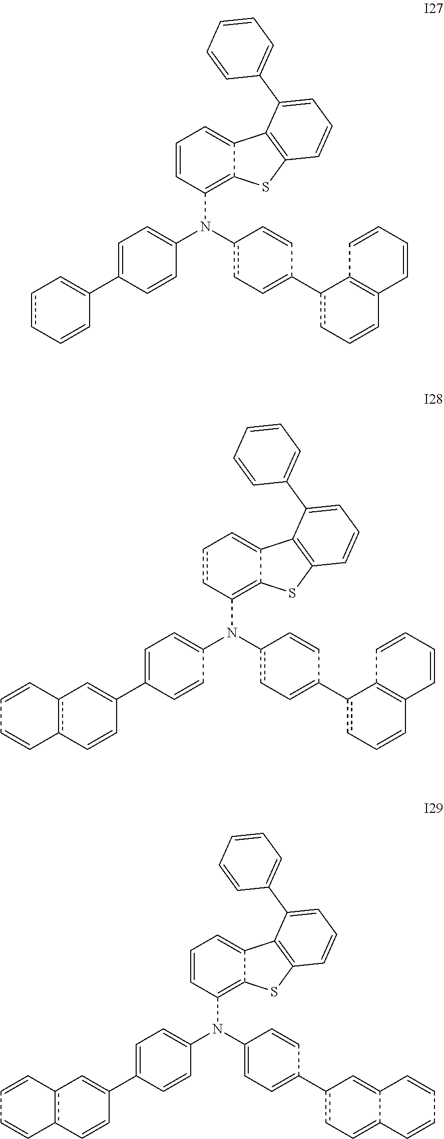

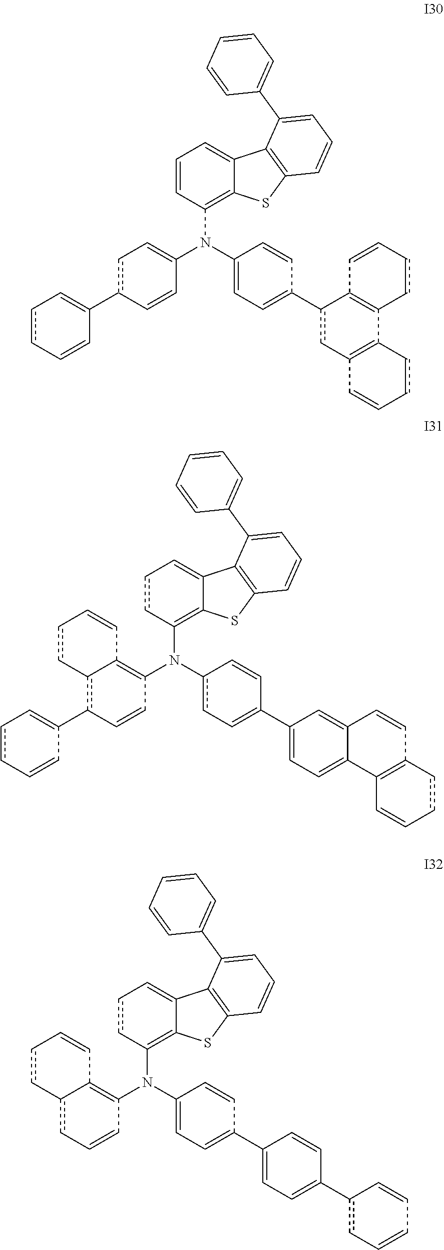

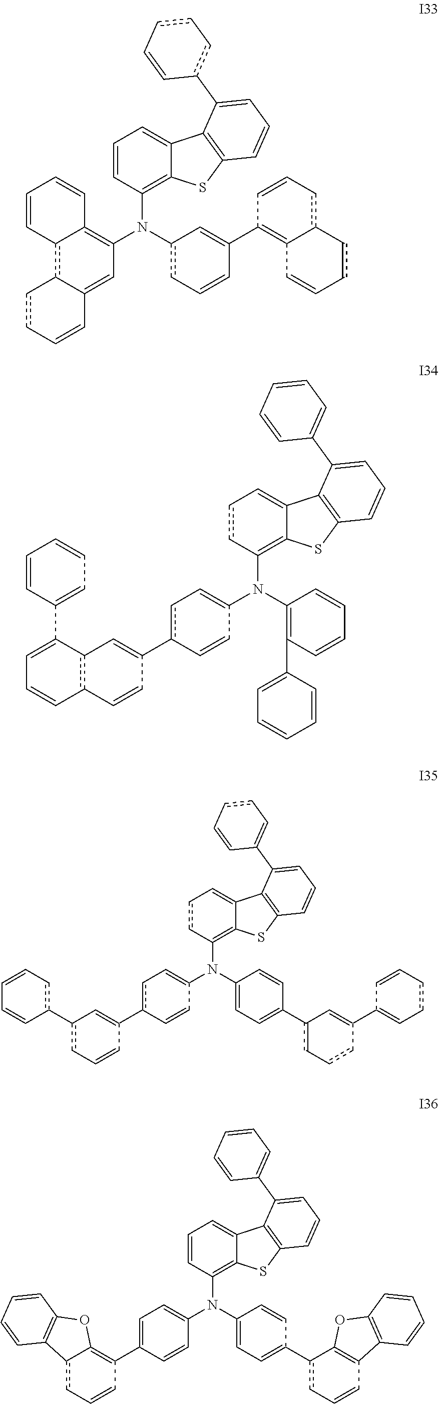

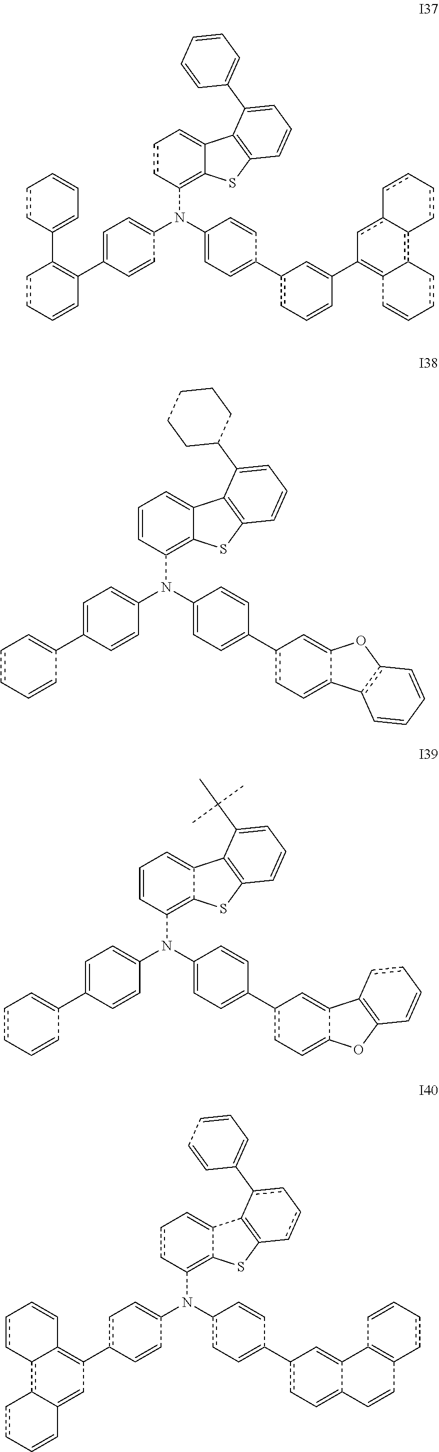

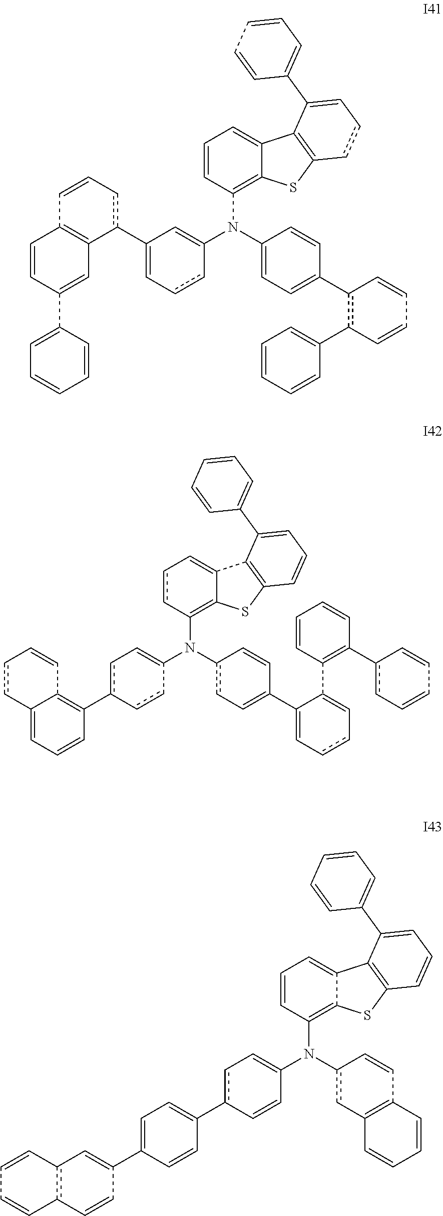

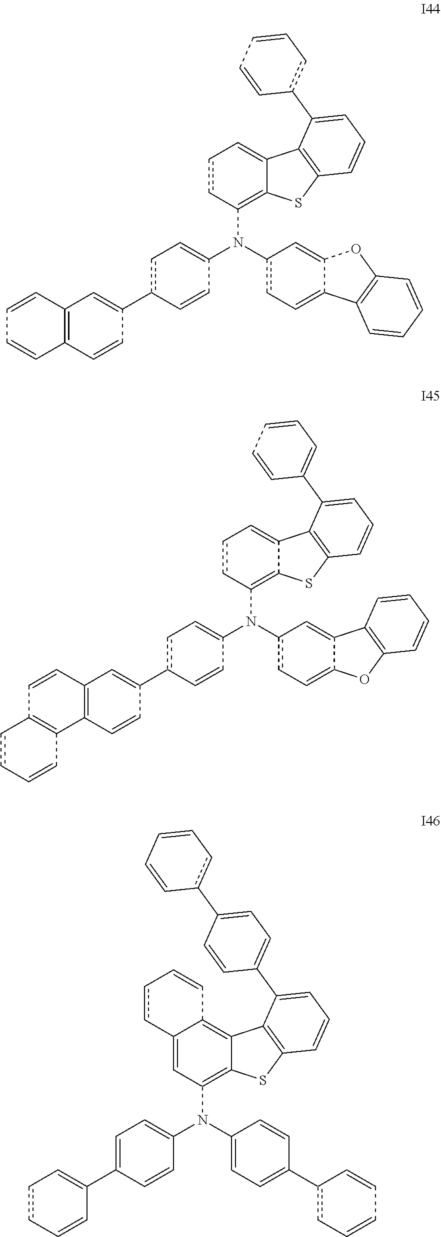

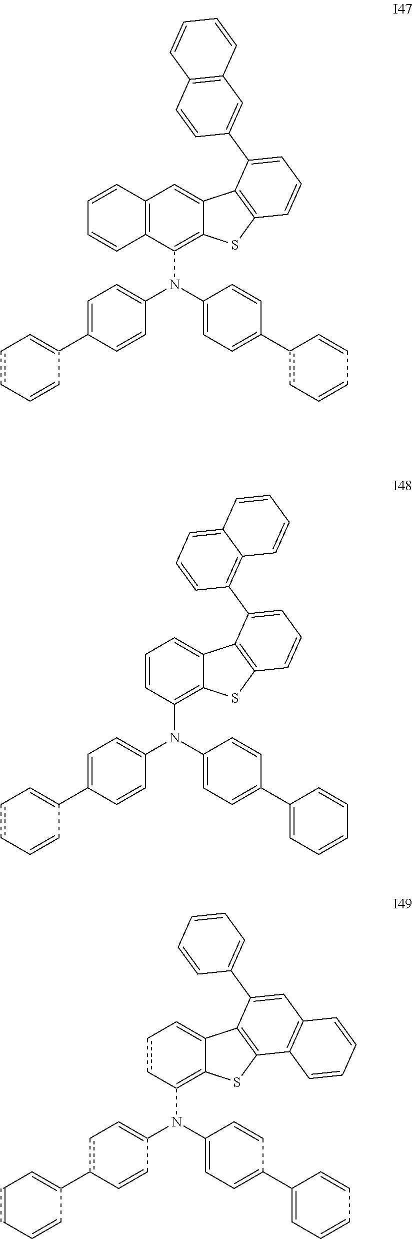

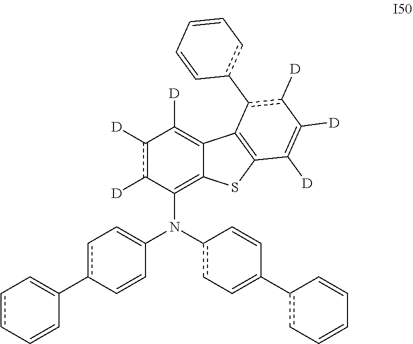

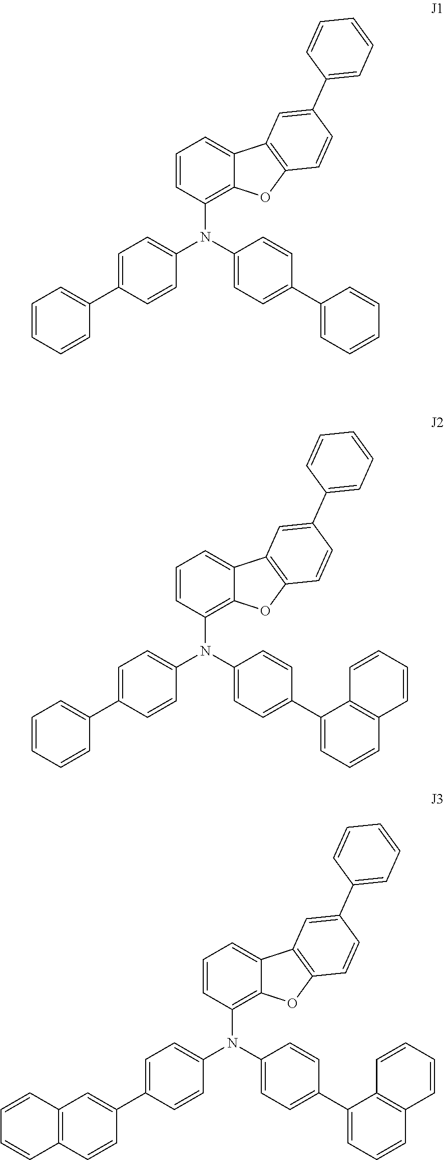 [SMILES: c1ccc(-c2ccc(N(c3ccc(-c4ccccc4)cc3)c3cccc4c3oc3ccc(-c5ccccc5)cc34)cc2)cc1 ; c1ccc(-c2ccc(N(c3ccc(-c4cccc5ccccc45)cc3)c3cccc4c3oc3ccc(-c5ccccc5)cc34)cc2)cc1 ; c1ccc(-c2ccc3oc4c(N(c5ccc(-c6ccc7ccccc7c6)cc5)c5ccc(-c6cccc7ccccc67)cc5)cccc4c3c2)cc1]

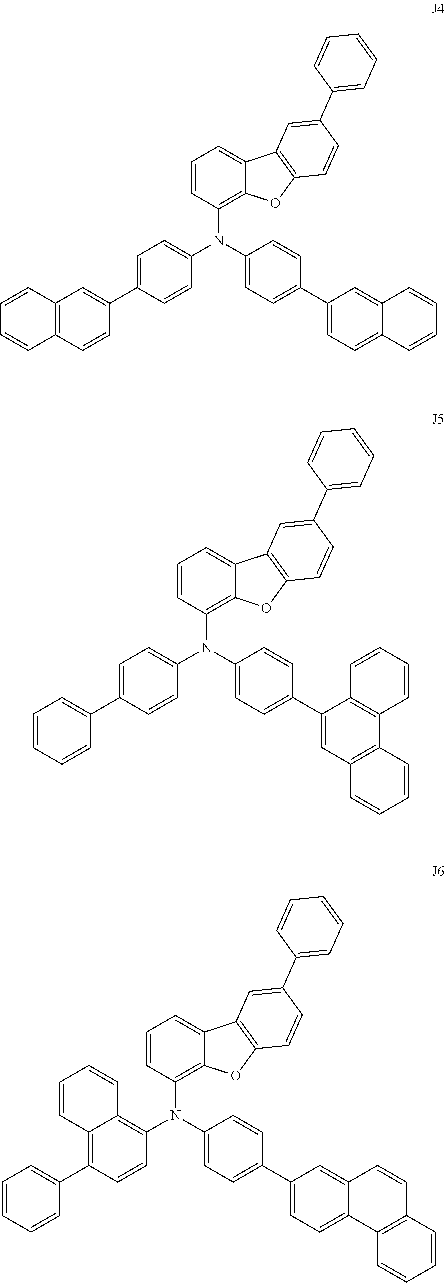 [SMILES: c1ccc(-c2ccc3oc4c(N(c5ccc(-c6ccc7ccccc7c6)cc5)c5ccc(-c6ccc7ccccc7c6)cc5)cccc4c3c2)cc1 ; c1ccc(-c2ccc(N(c3ccc(-c4cc5ccccc5c5ccccc45)cc3)c3cccc4c3oc3ccc(-c5ccccc5)cc34)cc2)cc1 ; c1ccc(-c2ccc3oc4c(N(c5ccc(-c6ccc7c(ccc8ccccc87)c6)cc5)c5ccc(-c6ccccc6)c6ccccc56)cccc4c3c2)cc1]

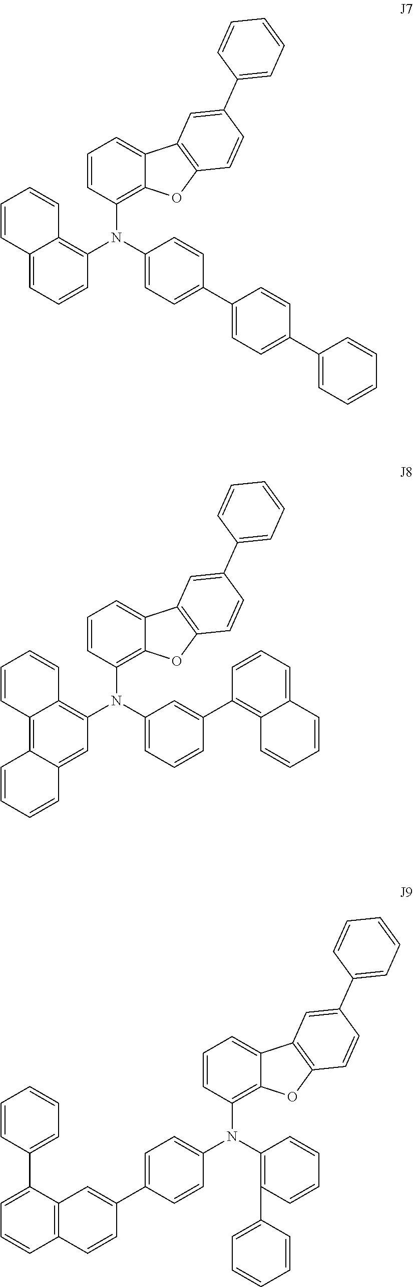 [SMILES: c1ccc(-c2ccc(-c3ccc(N(c4cccc5ccccc45)c4cccc5c4oc4ccc(-c6ccccc6)cc45)cc3)cc2)cc1 ; c1ccc(-c2ccc3oc4c(N(c5cccc(-c6cccc7ccccc67)c5)c5cc6ccccc6c6ccccc56)cccc4c3c2)cc1 ; c1ccc(-c2ccc3oc4c(N(c5ccc(-c6ccc7cccc(-c8ccccc8)c7c6)cc5)c5ccccc5-c5ccccc5)cccc4c3c2)cc1]

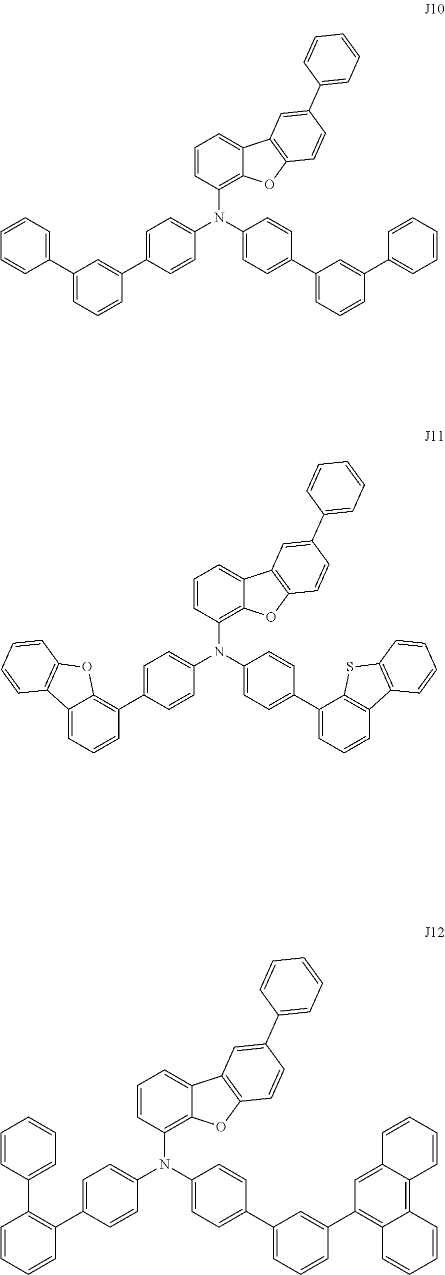 [SMILES: c1ccc(-c2cccc(-c3ccc(N(c4ccc(-c5cccc(-c6ccccc6)c5)cc4)c4cccc5c4oc4ccc(-c6ccccc6)cc45)cc3)c2)cc1 ; c1ccc(-c2ccc3oc4c(N(c5ccc(-c6cccc7c6oc6ccccc67)cc5)c5ccc(-c6cccc7c6sc6ccccc67)cc5)cccc4c3c2)cc1 ; c1ccc(-c2ccc3oc4c(N(c5ccc(-c6cccc(-c7cc8ccccc8c8ccccc78)c6)cc5)c5ccc(-c6ccccc6-c6ccccc6)cc5)cccc4c3c2)cc1]

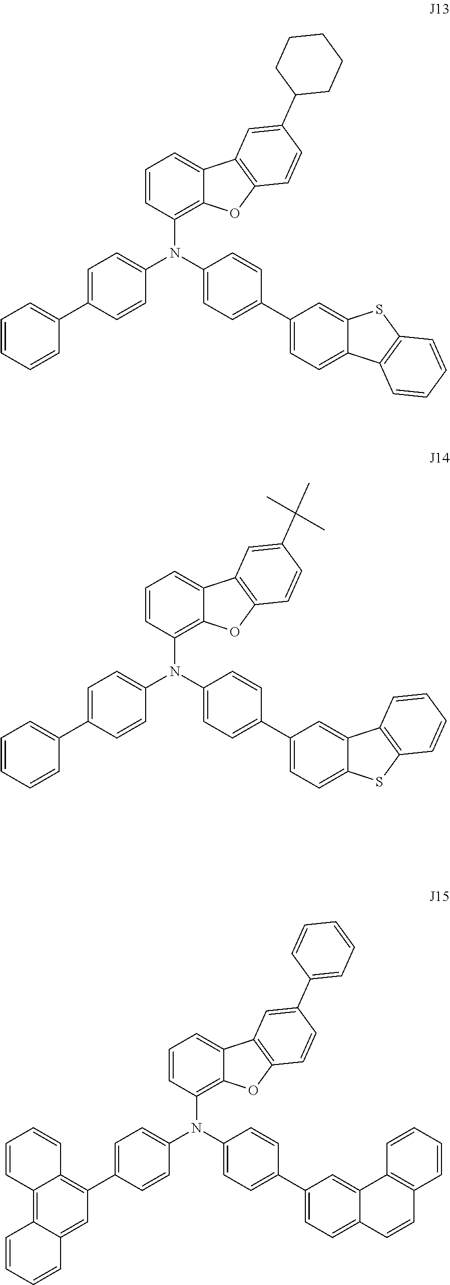 [SMILES: c1ccc(-c2ccc(N(c3ccc(-c4ccc5c(c4)sc4ccccc45)cc3)c3cccc4c3oc3ccc(C5CCCCC5)cc34)cc2)cc1 ; CC(C)(C)c1ccc2oc3c(N(c4ccc(-c5ccccc5)cc4)c4ccc(-c5ccc6sc7ccccc7c6c5)cc4)cccc3c2c1 ; c1ccc(-c2ccc3oc4c(N(c5ccc(-c6ccc7ccc8ccccc8c7c6)cc5)c5ccc(-c6cc7ccccc7c7ccccc67)cc5)cccc4c3c2)cc1]

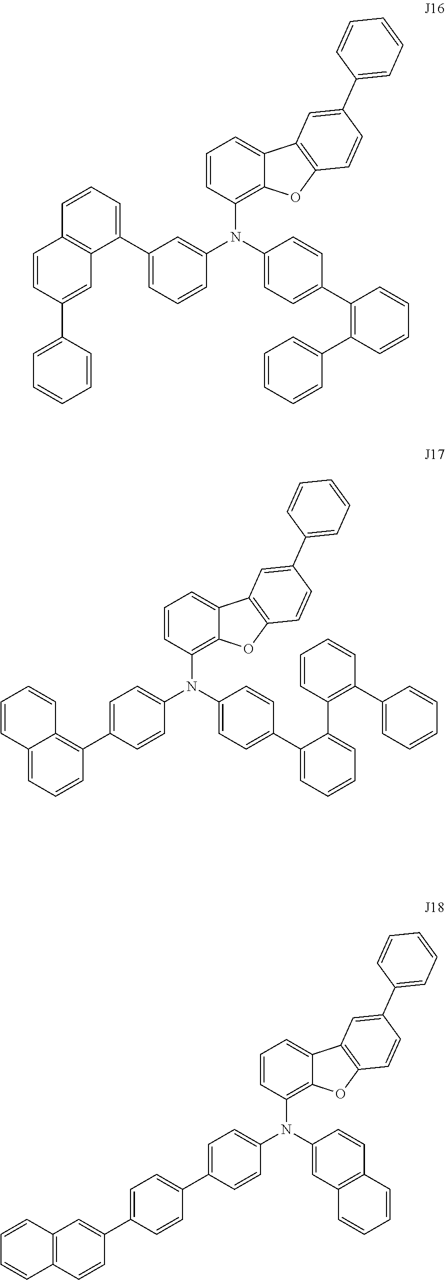 [SMILES: c1ccc(-c2ccc3cccc(-c4cccc(N(c5ccc(-c6ccccc6-c6ccccc6)cc5)c5cccc6c5oc5ccc(-c7ccccc7)cc56)c4)c3c2)cc1 ; c1ccc(-c2ccc3oc4c(N(c5ccc(-c6ccccc6-c6ccccc6-c6ccccc6)cc5)c5ccc(-c6cccc7ccccc67)cc5)cccc4c3c2)cc1 ; c1ccc(-c2ccc3oc4c(N(c5ccc(-c6ccc(-c7ccc8ccccc8c7)cc6)cc5)c5ccc6ccccc6c5)cccc4c3c2)cc1]

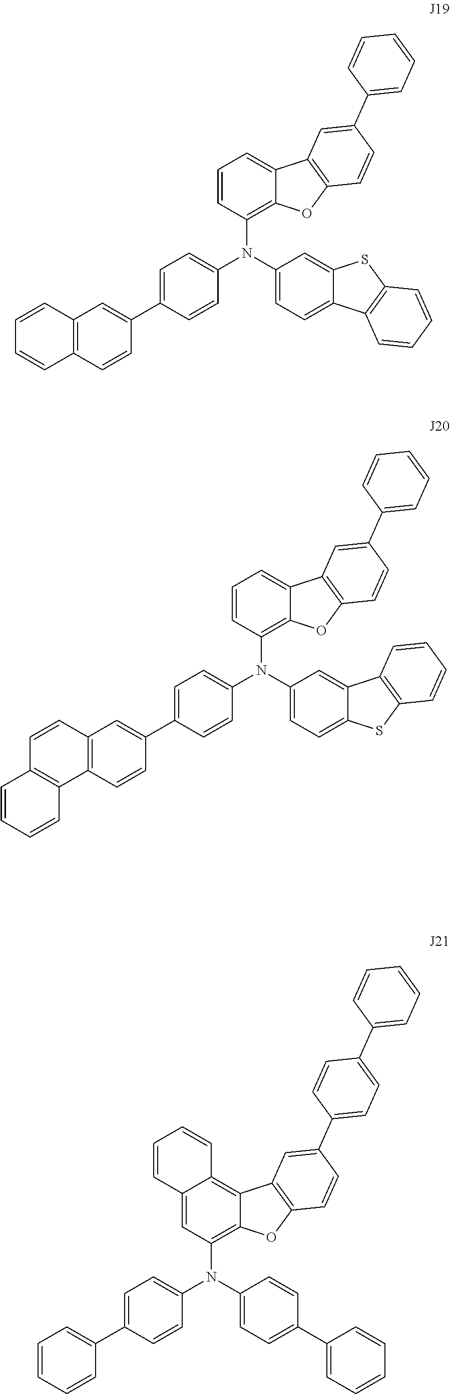 [SMILES: c1ccc(-c2ccc3oc4c(N(c5ccc(-c6ccc7ccccc7c6)cc5)c5ccc6c(c5)sc5ccccc56)cccc4c3c2)cc1 ; c1ccc(-c2ccc3oc4c(N(c5ccc(-c6ccc7c(ccc8ccccc87)c6)cc5)c5ccc6sc7ccccc7c6c5)cccc4c3c2)cc1 ; c1ccc(-c2ccc(-c3ccc4oc5c(N(c6ccc(-c7ccccc7)cc6)c6ccc(-c7ccccc7)cc6)cc6ccccc6c5c4c3)cc2)cc1]

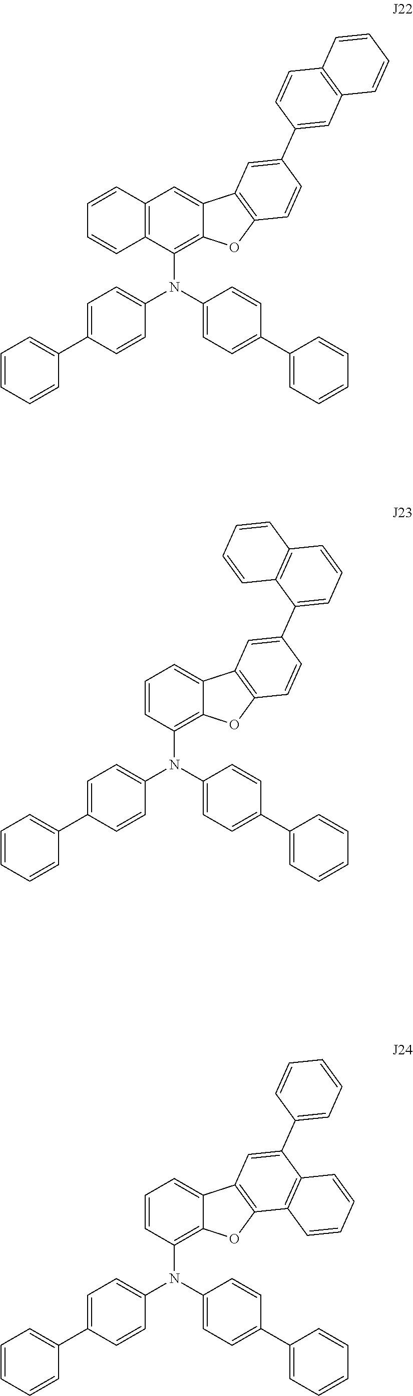 [SMILES: c1ccc(-c2ccc(N(c3ccc(-c4ccccc4)cc3)c3c4ccccc4cc4c3oc3ccc(-c5ccc6ccccc6c5)cc34)cc2)cc1 ; c1ccc(-c2ccc(N(c3ccc(-c4ccccc4)cc3)c3cccc4c3oc3ccc(-c5cccc6ccccc56)cc34)cc2)cc1 ; c1ccc(-c2ccc(N(c3ccc(-c4ccccc4)cc3)c3cccc4c3oc3c5ccccc5c(-c5ccccc5)cc43)cc2)cc1]

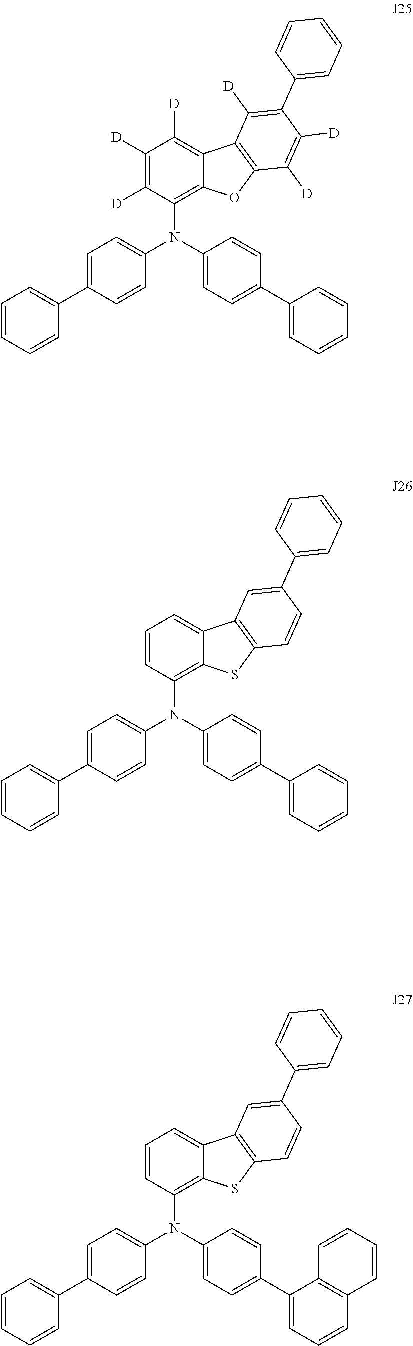 [SMILES: [2H]c1c(-c2ccccc2)c([2H])c2c(oc3c(N(c4ccc(-c5ccccc5)cc4)c4ccc(-c5ccccc5)cc4)c([2H])c([2H])c([2H])c32)c1[2H] ; c1ccc(-c2ccc(N(c3ccc(-c4ccccc4)cc3)c3cccc4c3sc3ccc(-c5ccccc5)cc34)cc2)cc1 ; c1ccc(-c2ccc(N(c3ccc(-c4cccc5ccccc45)cc3)c3cccc4c3sc3ccc(-c5ccccc5)cc34)cc2)cc1]

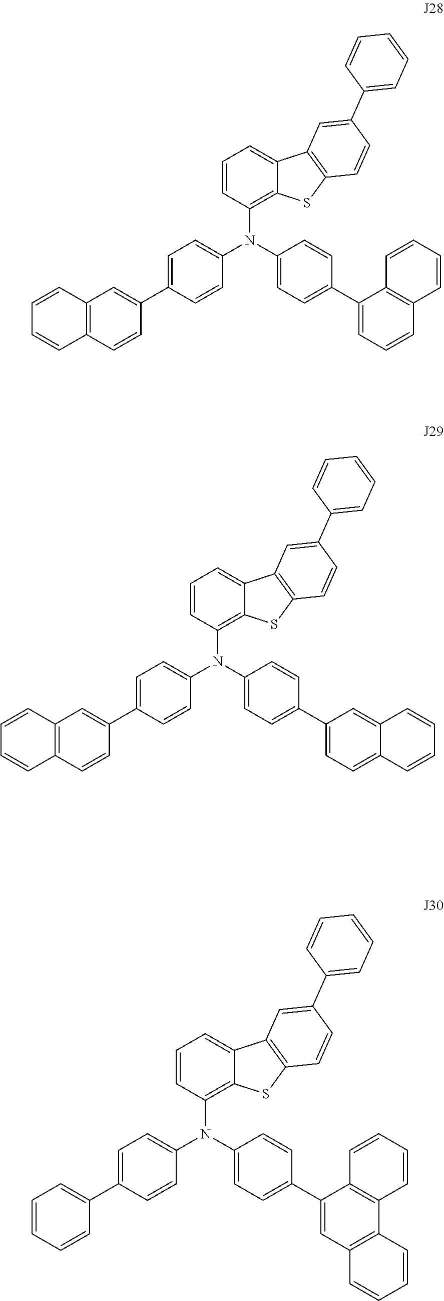 [SMILES: c1ccc(-c2ccc3sc4c(N(c5ccc(-c6ccc7ccccc7c6)cc5)c5ccc(-c6cccc7ccccc67)cc5)cccc4c3c2)cc1 ; c1ccc(-c2ccc3sc4c(N(c5ccc(-c6ccc7ccccc7c6)cc5)c5ccc(-c6ccc7ccccc7c6)cc5)cccc4c3c2)cc1 ; c1ccc(-c2ccc(N(c3ccc(-c4cc5ccccc5c5ccccc45)cc3)c3cccc4c3sc3ccc(-c5ccccc5)cc34)cc2)cc1]

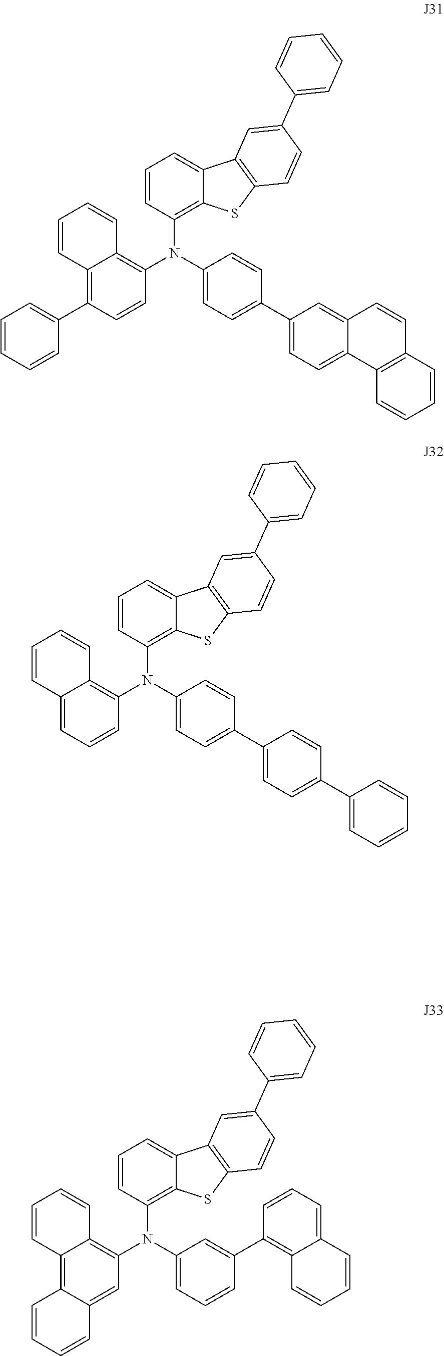 [SMILES: c1ccc(-c2ccc3sc4c(N(c5ccc(-c6ccc7c(ccc8ccccc87)c6)cc5)c5ccc(-c6ccccc6)c6ccccc56)cccc4c3c2)cc1 ; c1ccc(-c2ccc(-c3ccc(N(c4cccc5ccccc45)c4cccc5c4sc4ccc(-c6ccccc6)cc45)cc3)cc2)cc1 ; c1ccc(-c2ccc3sc4c(N(c5cccc(-c6cccc7ccccc67)c5)c5cc6ccccc6c6ccccc56)cccc4c3c2)cc1]

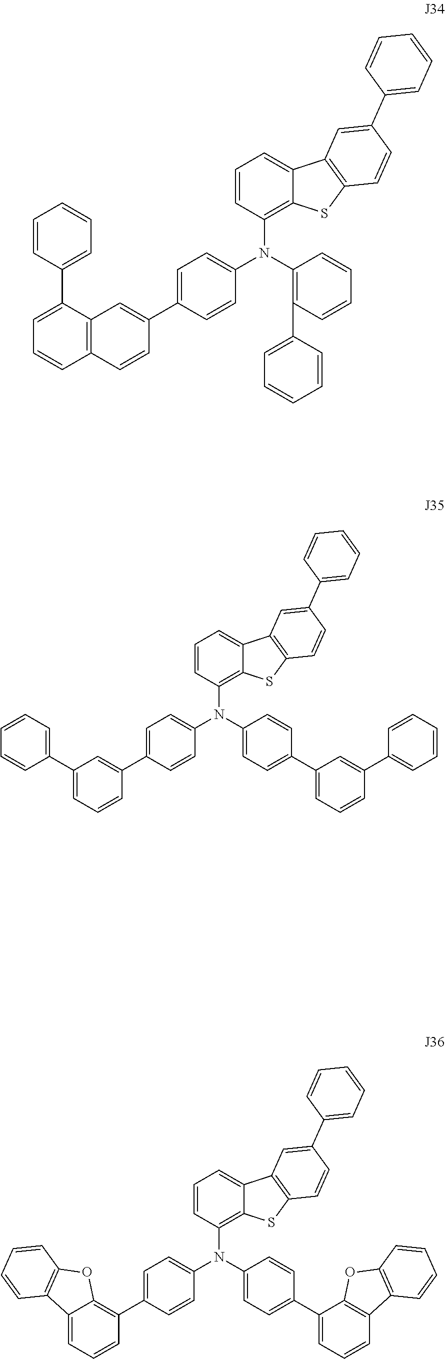 [SMILES: c1ccc(-c2ccc3sc4c(N(c5ccc(-c6ccc7cccc(-c8ccccc8)c7c6)cc5)c5ccccc5-c5ccccc5)cccc4c3c2)cc1 ; c1ccc(-c2cccc(-c3ccc(N(c4ccc(-c5cccc(-c6ccccc6)c5)cc4)c4cccc5c4sc4ccc(-c6ccccc6)cc45)cc3)c2)cc1 ; c1ccc(-c2ccc3sc4c(N(c5ccc(-c6cccc7c6oc6ccccc67)cc5)c5ccc(-c6cccc7c6oc6ccccc67)cc5)cccc4c3c2)cc1]

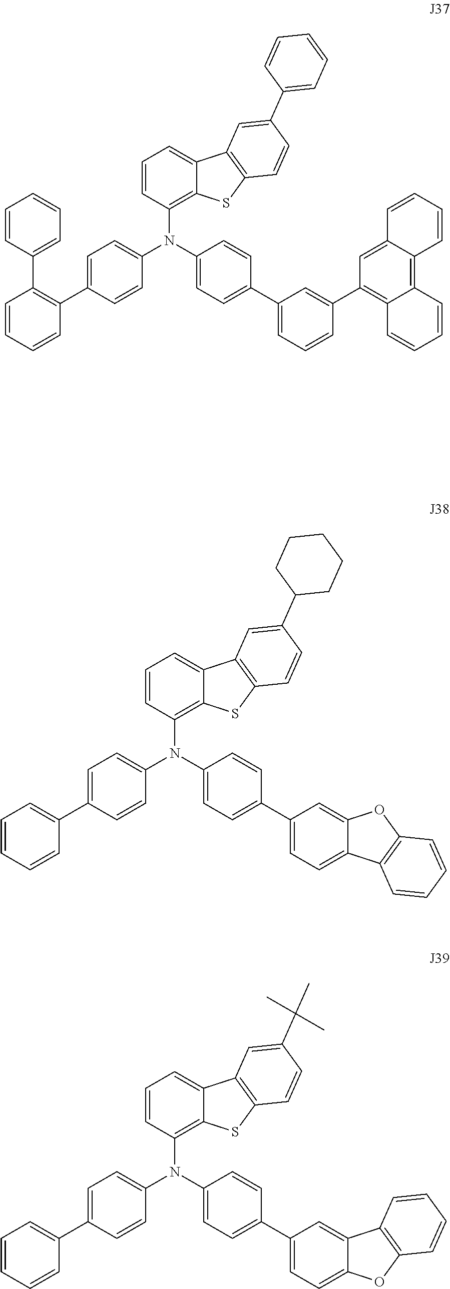 [SMILES: c1ccc(-c2ccc3sc4c(N(c5ccc(-c6cccc(-c7cc8ccccc8c8ccccc78)c6)cc5)c5ccc(-c6ccccc6-c6ccccc6)cc5)cccc4c3c2)cc1 ; c1ccc(-c2ccc(N(c3ccc(-c4ccc5c(c4)oc4ccccc45)cc3)c3cccc4c3sc3ccc(C5CCCCC5)cc34)cc2)cc1 ; CC(C)(C)c1ccc2sc3c(N(c4ccc(-c5ccccc5)cc4)c4ccc(-c5ccc6oc7ccccc7c6c5)cc4)cccc3c2c1]

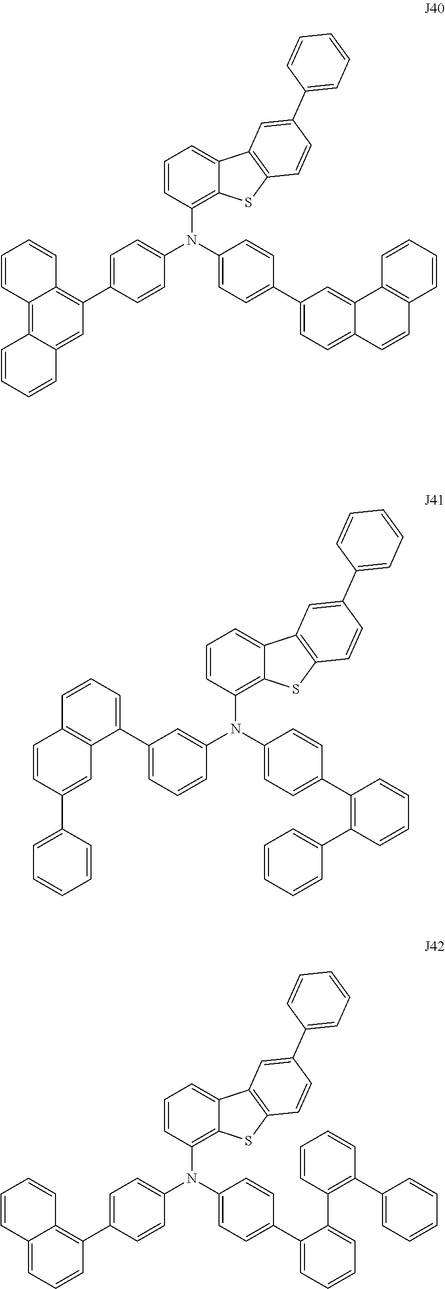 [SMILES: c1ccc(-c2ccc3sc4c(N(c5ccc(-c6ccc7ccc8ccccc8c7c6)cc5)c5ccc(-c6cc7ccccc7c7ccccc67)cc5)cccc4c3c2)cc1 ; c1ccc(-c2ccc3cccc(-c4cccc(N(c5ccc(-c6ccccc6-c6ccccc6)cc5)c5cccc6c5sc5ccc(-c7ccccc7)cc56)c4)c3c2)cc1 ; c1ccc(-c2ccc3sc4c(N(c5ccc(-c6ccccc6-c6ccccc6-c6ccccc6)cc5)c5ccc(-c6cccc7ccccc67)cc5)cccc4c3c2)cc1]

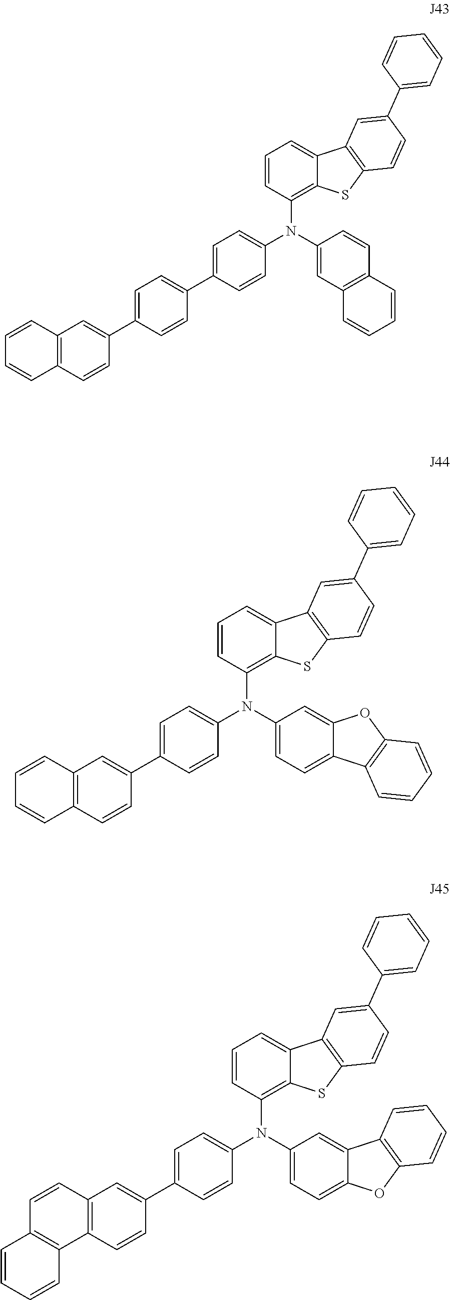 [SMILES: c1ccc(-c2ccc3sc4c(N(c5ccc(-c6ccc(-c7ccc8ccccc8c7)cc6)cc5)c5ccc6ccccc6c5)cccc4c3c2)cc1 ; c1ccc(-c2ccc3sc4c(N(c5ccc(-c6ccc7ccccc7c6)cc5)c5ccc6c(c5)oc5ccccc56)cccc4c3c2)cc1 ; c1ccc(-c2ccc3sc4c(N(c5ccc(-c6ccc7c(ccc8ccccc87)c6)cc5)c5ccc6oc7ccccc7c6c5)cccc4c3c2)cc1]

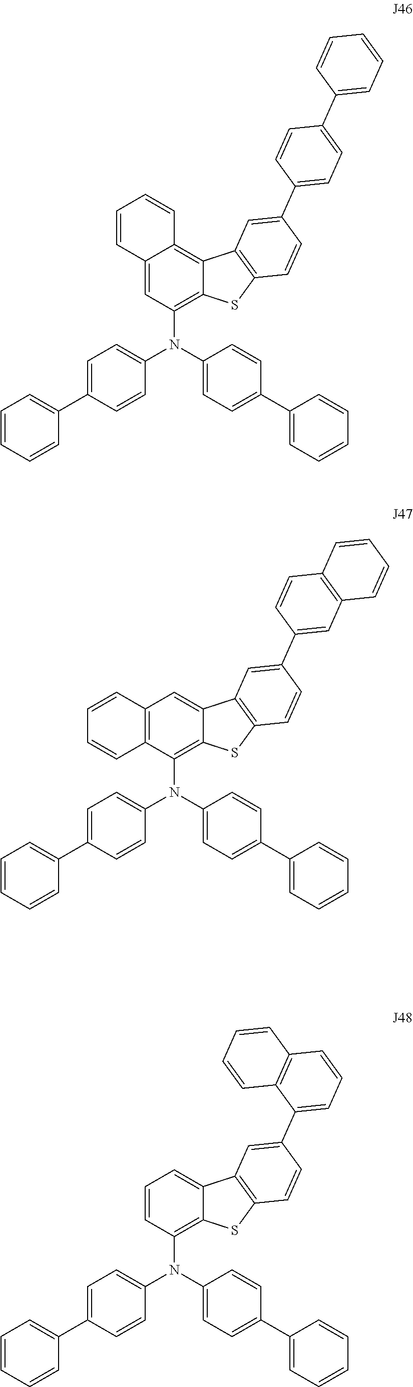 [SMILES: c1ccc(-c2ccc(-c3ccc4sc5c(N(c6ccc(-c7ccccc7)cc6)c6ccc(-c7ccccc7)cc6)cc6ccccc6c5c4c3)cc2)cc1 ; c1ccc(-c2ccc(N(c3ccc(-c4ccccc4)cc3)c3c4ccccc4cc4c3sc3ccc(-c5ccc6ccccc6c5)cc34)cc2)cc1 ; c1ccc(-c2ccc(N(c3ccc(-c4ccccc4)cc3)c3cccc4c3sc3ccc(-c5cccc6ccccc56)cc34)cc2)cc1]

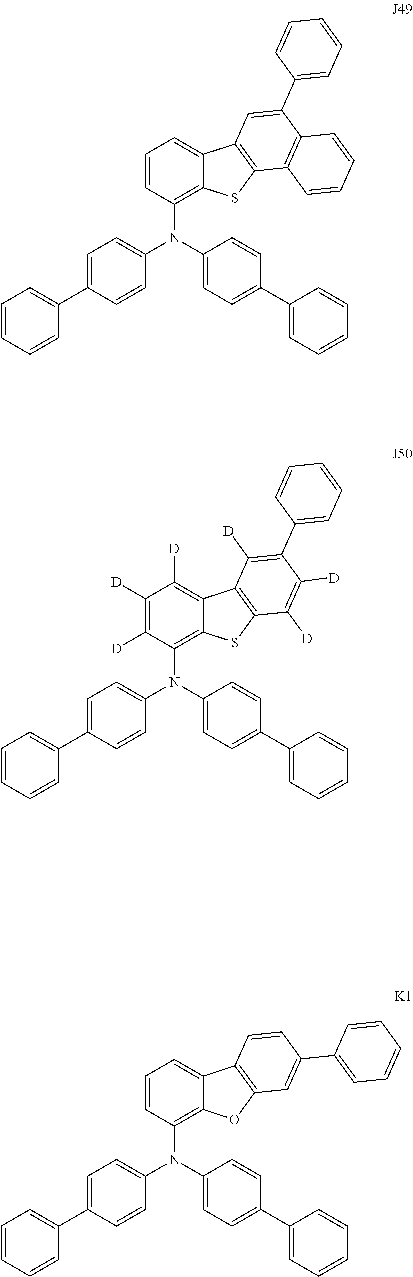 [SMILES: c1ccc(-c2ccc(N(c3ccc(-c4ccccc4)cc3)c3cccc4c3sc3c5ccccc5c(-c5ccccc5)cc43)cc2)cc1 ; [2H]c1c(-c2ccccc2)c([2H])c2c(sc3c(N(c4ccc(-c5ccccc5)cc4)c4ccc(-c5ccccc5)cc4)c([2H])c([2H])c([2H])c32)c1[2H] ; c1ccc(-c2ccc(N(c3ccc(-c4ccccc4)cc3)c3cccc4c3oc3cc(-c5ccccc5)ccc34)cc2)cc1]

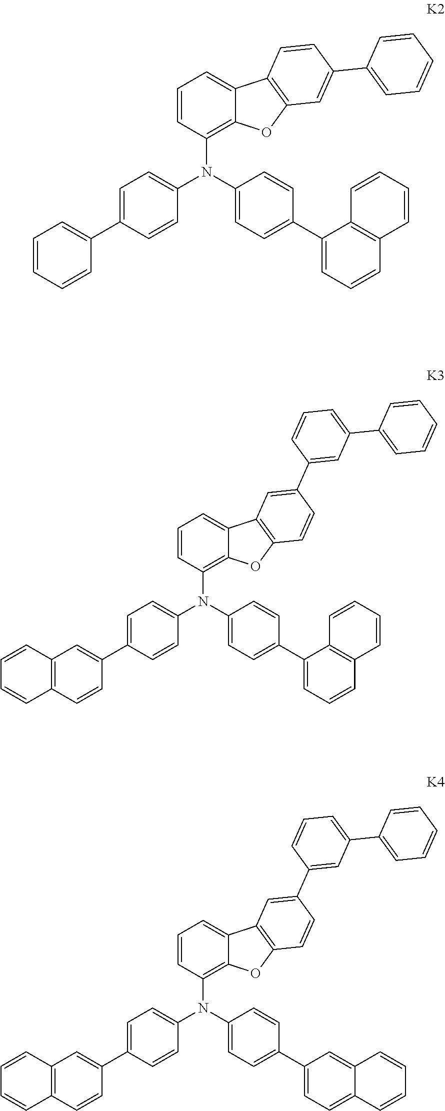 [SMILES: c1ccc(-c2ccc(N(c3ccc(-c4cccc5ccccc45)cc3)c3cccc4c3oc3cc(-c5ccccc5)ccc34)cc2)cc1 ; c1ccc(-c2cccc(-c3ccc4oc5c(N(c6ccc(-c7ccc8ccccc8c7)cc6)c6ccc(-c7cccc8ccccc78)cc6)cccc5c4c3)c2)cc1 ; c1ccc(-c2cccc(-c3ccc4oc5c(N(c6ccc(-c7ccc8ccccc8c7)cc6)c6ccc(-c7ccc8ccccc8c7)cc6)cccc5c4c3)c2)cc1]

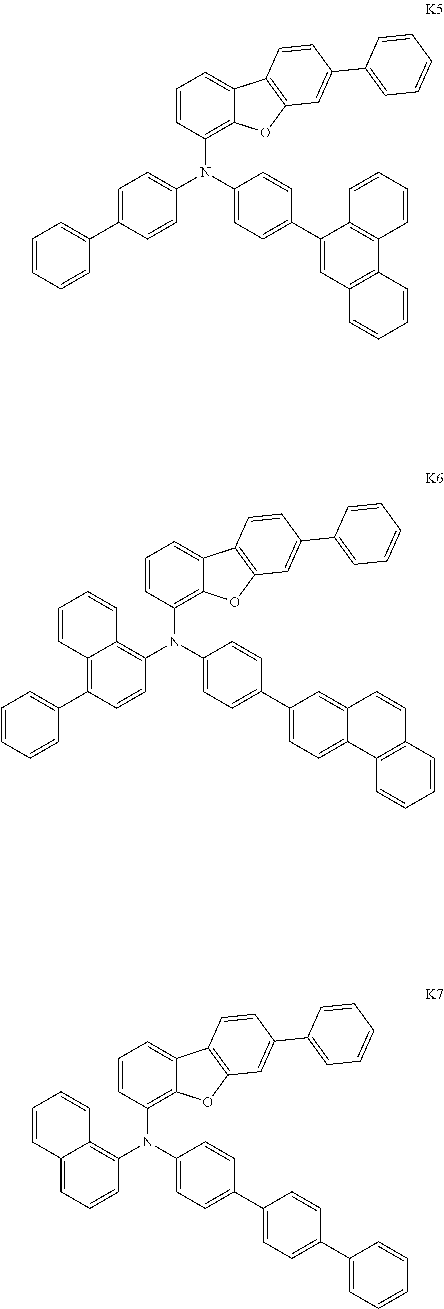 [SMILES: c1ccc(-c2ccc(N(c3ccc(-c4cc5ccccc5c5ccccc45)cc3)c3cccc4c3oc3cc(-c5ccccc5)ccc34)cc2)cc1 ; c1ccc(-c2ccc3c(c2)oc2c(N(c4ccc(-c5ccc6c(ccc7ccccc76)c5)cc4)c4ccc(-c5ccccc5)c5ccccc45)cccc23)cc1 ; c1ccc(-c2ccc(-c3ccc(N(c4cccc5ccccc45)c4cccc5c4oc4cc(-c6ccccc6)ccc45)cc3)cc2)cc1]

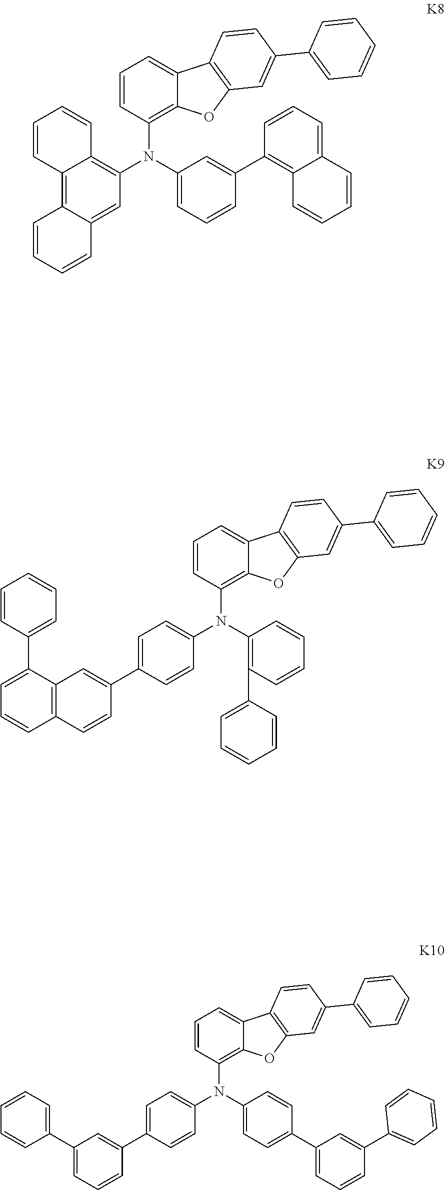 [SMILES: c1ccc(-c2ccc3c(c2)oc2c(N(c4cccc(-c5cccc6ccccc56)c4)c4cc5ccccc5c5ccccc45)cccc23)cc1 ; c1ccc(-c2ccc3c(c2)oc2c(N(c4ccc(-c5ccc6cccc(-c7ccccc7)c6c5)cc4)c4ccccc4-c4ccccc4)cccc23)cc1 ; c1ccc(-c2cccc(-c3ccc(N(c4ccc(-c5cccc(-c6ccccc6)c5)cc4)c4cccc5c4oc4cc(-c6ccccc6)ccc45)cc3)c2)cc1]

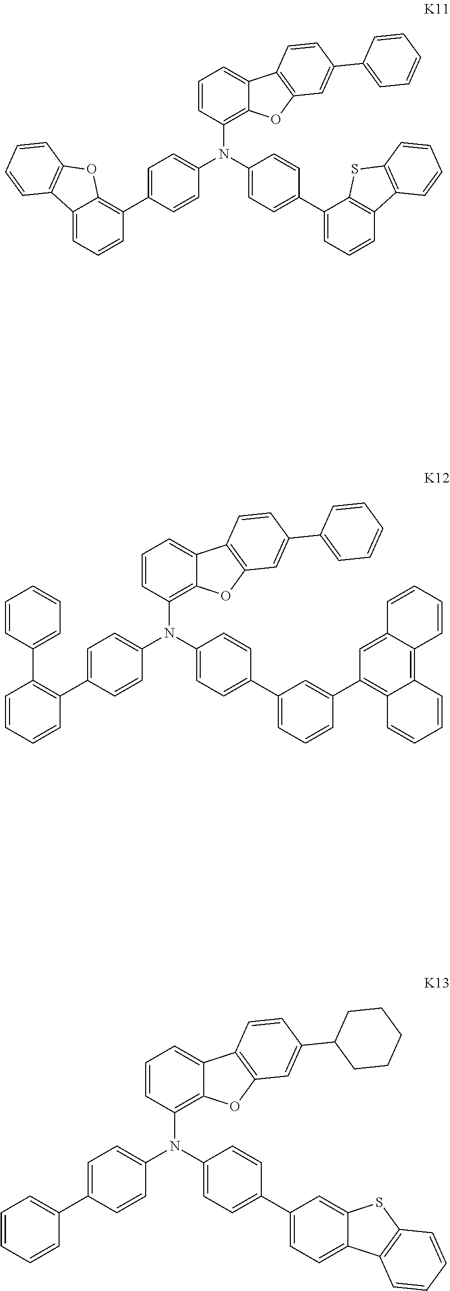 [SMILES: c1ccc(-c2ccc3c(c2)oc2c(N(c4ccc(-c5cccc6c5oc5ccccc56)cc4)c4ccc(-c5cccc6c5sc5ccccc56)cc4)cccc23)cc1 ; c1ccc(-c2ccc3c(c2)oc2c(N(c4ccc(-c5cccc(-c6cc7ccccc7c7ccccc67)c5)cc4)c4ccc(-c5ccccc5-c5ccccc5)cc4)cccc23)cc1 ; c1ccc(-c2ccc(N(c3ccc(-c4ccc5c(c4)sc4ccccc45)cc3)c3cccc4c3oc3cc(C5CCCCC5)ccc34)cc2)cc1]

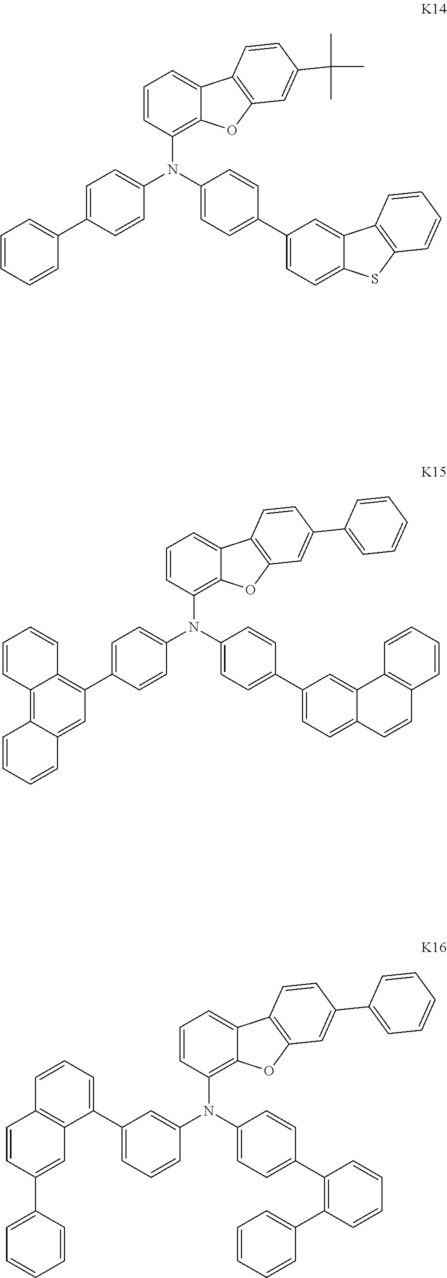 [SMILES: CC(C)(C)c1ccc2c(c1)oc1c(N(c3ccc(-c4ccccc4)cc3)c3ccc(-c4ccc5sc6ccccc6c5c4)cc3)cccc12 ; c1ccc(-c2ccc3c(c2)oc2c(N(c4ccc(-c5ccc6ccc7ccccc7c6c5)cc4)c4ccc(-c5cc6ccccc6c6ccccc56)cc4)cccc23)cc1 ; c1ccc(-c2ccc3c(c2)oc2c(N(c4ccc(-c5ccccc5-c5ccccc5)cc4)c4cccc(-c5cccc6ccc(-c7ccccc7)cc56)c4)cccc23)cc1]

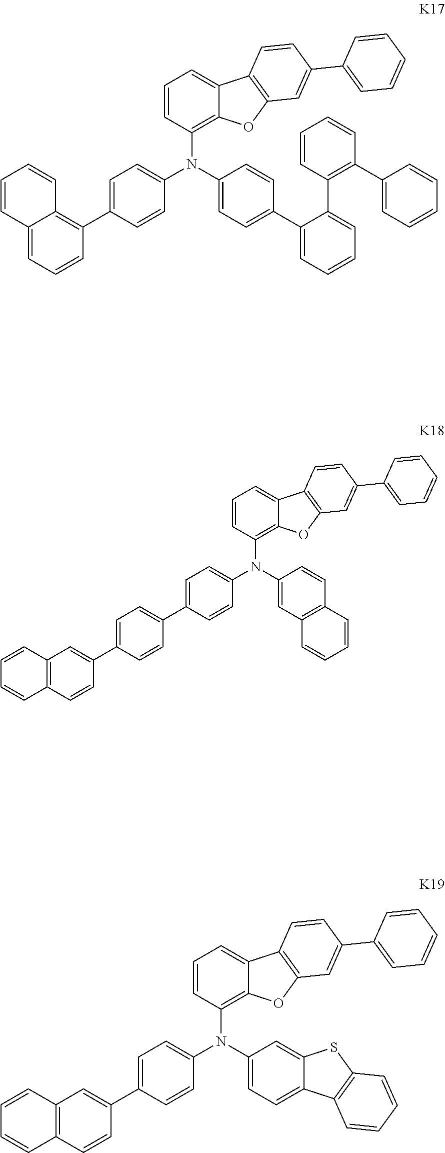 [SMILES: c1ccc(-c2ccc3c(c2)oc2c(N(c4ccc(-c5ccccc5-c5ccccc5-c5ccccc5)cc4)c4ccc(-c5cccc6ccccc56)cc4)cccc23)cc1 ; c1ccc(-c2ccc3c(c2)oc2c(N(c4ccc(-c5ccc(-c6ccc7ccccc7c6)cc5)cc4)c4ccc5ccccc5c4)cccc23)cc1 ; c1ccc(-c2ccc3c(c2)oc2c(N(c4ccc(-c5ccc6ccccc6c5)cc4)c4ccc5c(c4)sc4ccccc45)cccc23)cc1]

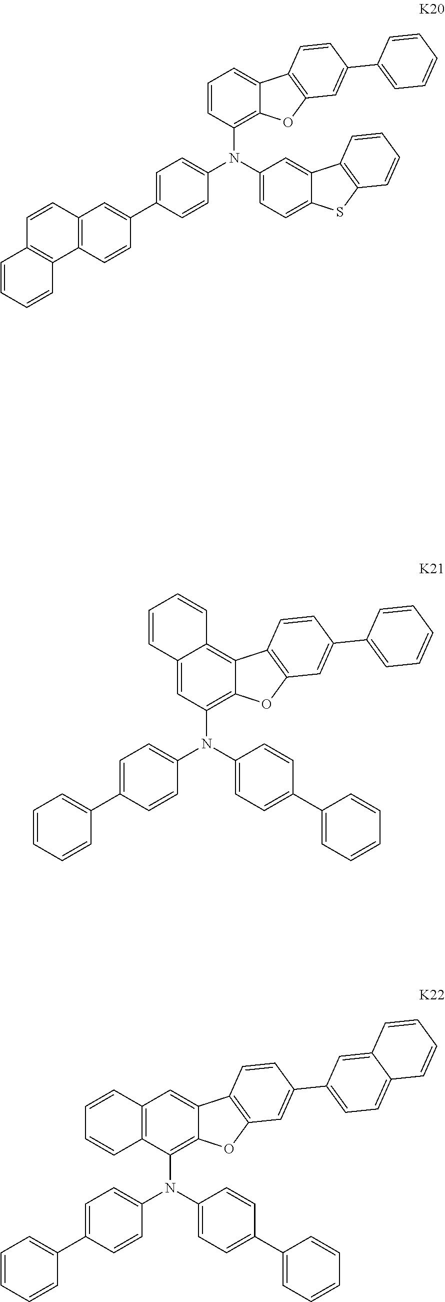 [SMILES: c1ccc(-c2ccc3c(c2)oc2c(N(c4ccc(-c5ccc6c(ccc7ccccc76)c5)cc4)c4ccc5sc6ccccc6c5c4)cccc23)cc1 ; c1ccc(-c2ccc(N(c3ccc(-c4ccccc4)cc3)c3cc4ccccc4c4c3oc3cc(-c5ccccc5)ccc34)cc2)cc1 ; c1ccc(-c2ccc(N(c3ccc(-c4ccccc4)cc3)c3c4ccccc4cc4c3oc3cc(-c5ccc6ccccc6c5)ccc34)cc2)cc1]

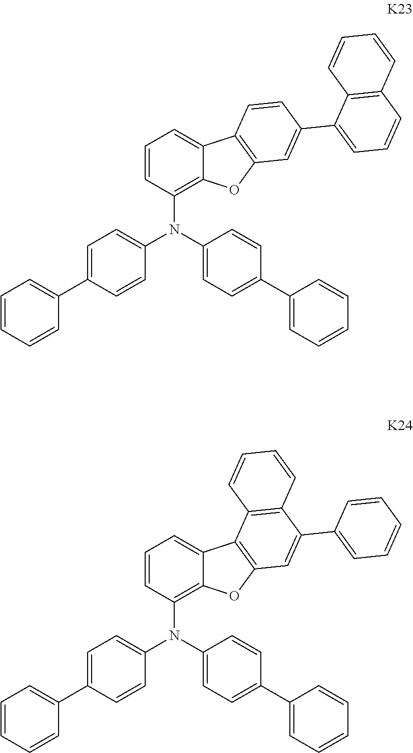 [SMILES: c1ccc(-c2ccc(N(c3ccc(-c4ccccc4)cc3)c3cccc4c3oc3cc(-c5cccc6ccccc56)ccc34)cc2)cc1 ; c1ccc(-c2ccc(N(c3ccc(-c4ccccc4)cc3)c3cccc4c3oc3cc(-c5ccccc5)c5ccccc5c34)cc2)cc1]

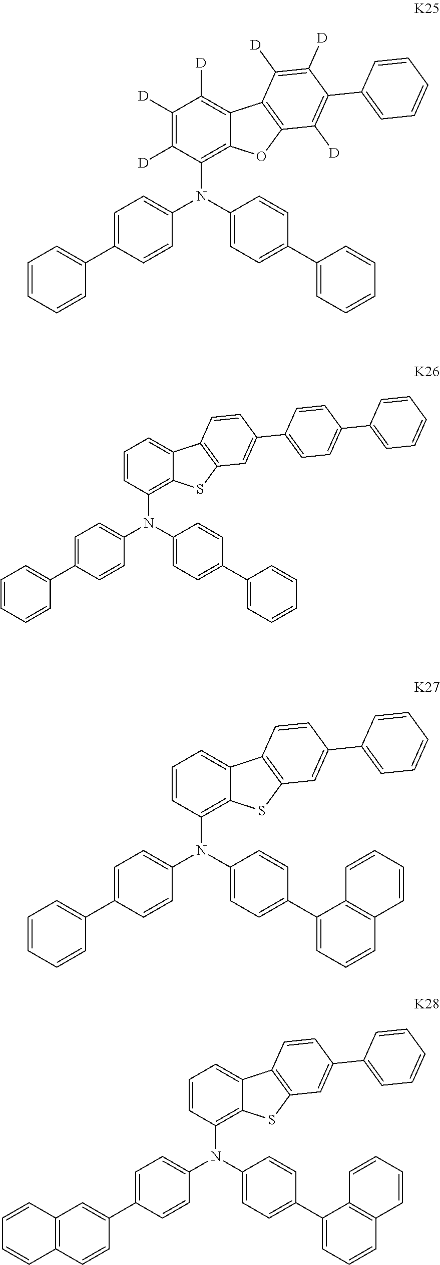 [SMILES: [2H]c1c([2H])c([2H])c2c(oc3c([2H])c(-c4ccccc4)c([2H])c([2H])c32)c1N(c1ccc(-c2ccccc2)cc1)c1ccc(-c2ccccc2)cc1 ; c1ccc(-c2ccc(-c3ccc4c(c3)sc3c(N(c5ccc(-c6ccccc6)cc5)c5ccc(-c6ccccc6)cc5)cccc34)cc2)cc1 ; c1ccc(-c2ccc(N(c3ccc(-c4cccc5ccccc45)cc3)c3cccc4c3sc3cc(-c5ccccc5)ccc34)cc2)cc1 ; c1ccc(-c2ccc3c(c2)sc2c(N(c4ccc(-c5ccc6ccccc6c5)cc4)c4ccc(-c5cccc6ccccc56)cc4)cccc23)cc1]

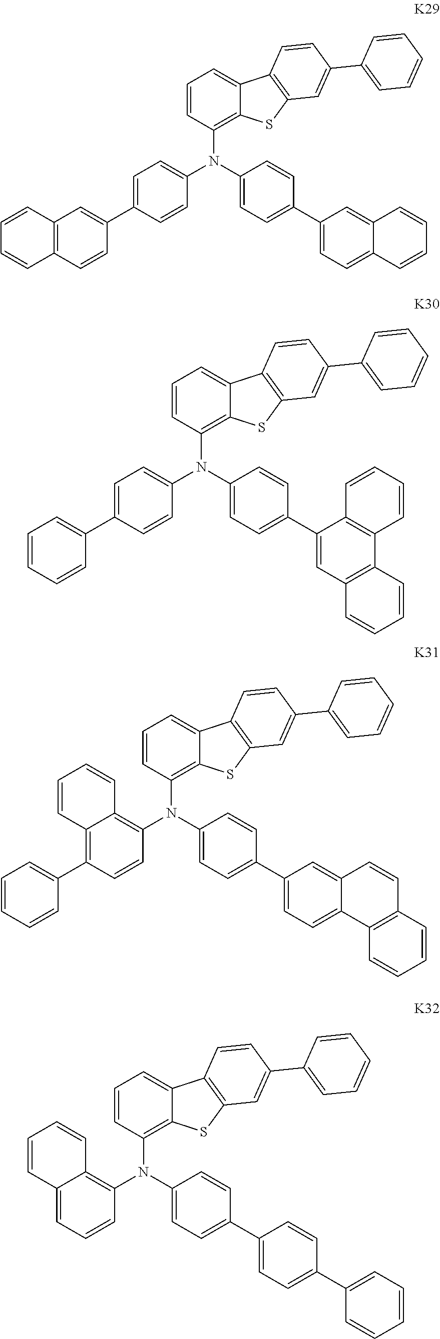

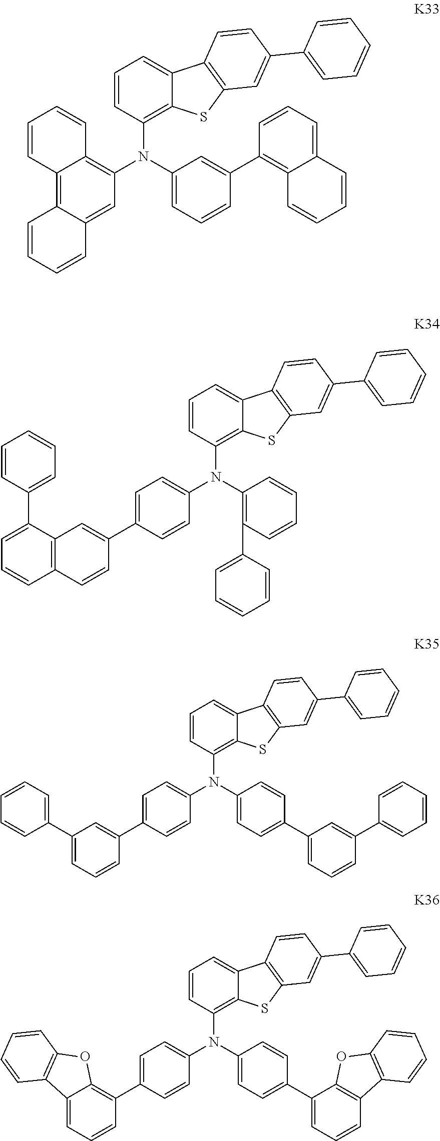 [SMILES: c1ccc(-c2ccc3c(c2)sc2c(N(c4cccc(-c5cccc6ccccc56)c4)c4cc5ccccc5c5ccccc45)cccc23)cc1 ; c1ccc(-c2ccc3c(c2)sc2c(N(c4ccc(-c5ccc6cccc(-c7ccccc7)c6c5)cc4)c4ccccc4-c4ccccc4)cccc23)cc1 ; c1ccc(-c2cccc(-c3ccc(N(c4ccc(-c5cccc(-c6ccccc6)c5)cc4)c4cccc5c4sc4cc(-c6ccccc6)ccc45)cc3)c2)cc1 ; c1ccc(-c2ccc3c(c2)sc2c(N(c4ccc(-c5cccc6c5oc5ccccc56)cc4)c4ccc(-c5cccc6c5oc5ccccc56)cc4)cccc23)cc1]

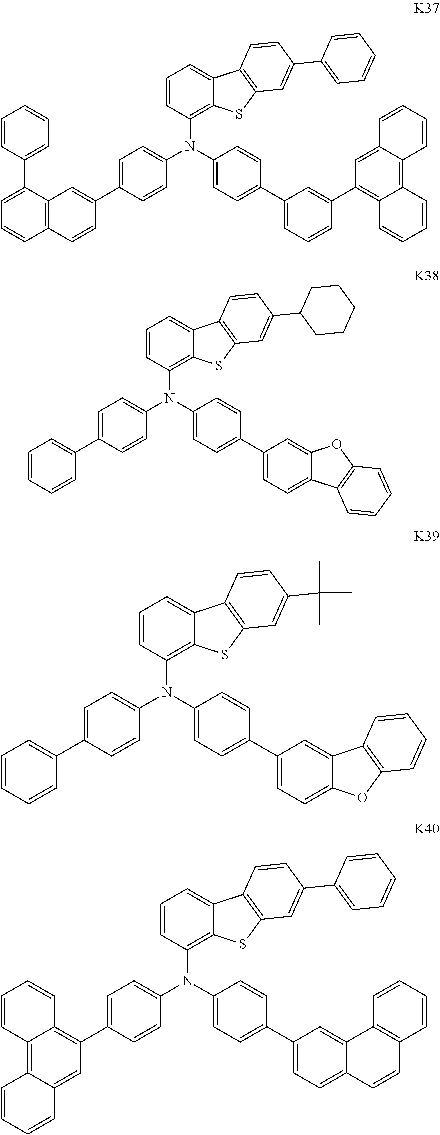 [SMILES: c1ccc(-c2ccc3c(c2)sc2c(N(c4ccc(-c5cccc(-c6cc7ccccc7c7ccccc67)c5)cc4)c4ccc(-c5ccc6cccc(-c7ccccc7)c6c5)cc4)cccc23)cc1 ; c1ccc(-c2ccc(N(c3ccc(-c4ccc5c(c4)oc4ccccc45)cc3)c3cccc4c3sc3cc(C5CCCCC5)ccc34)cc2)cc1 ; CC(C)(C)c1ccc2c(c1)sc1c(N(c3ccc(-c4ccccc4)cc3)c3ccc(-c4ccc5oc6ccccc6c5c4)cc3)cccc12 ; c1ccc(-c2ccc3c(c2)sc2c(N(c4ccc(-c5ccc6ccc7ccccc7c6c5)cc4)c4ccc(-c5cc6ccccc6c6ccccc56)cc4)cccc23)cc1]

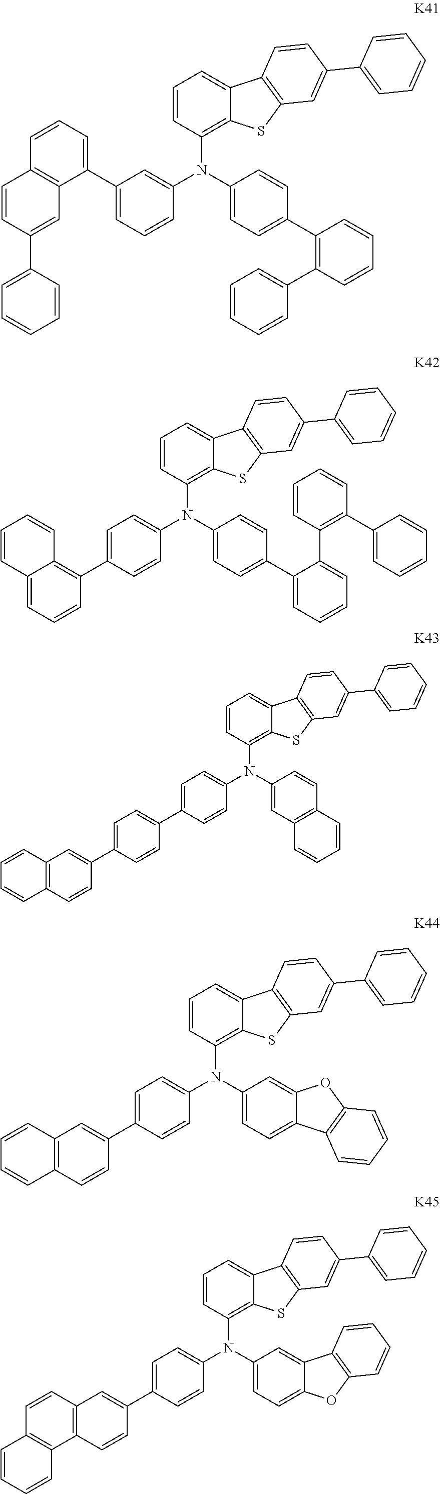 [SMILES: c1ccc(-c2ccc3c(c2)sc2c(N(c4ccc(-c5ccccc5-c5ccccc5)cc4)c4cccc(-c5cccc6ccc(-c7ccccc7)cc56)c4)cccc23)cc1 ; c1ccc(-c2ccc3c(c2)sc2c(N(c4ccc(-c5ccccc5-c5ccccc5-c5ccccc5)cc4)c4ccc(-c5cccc6ccccc56)cc4)cccc23)cc1 ; c1ccc(-c2ccc3c(c2)sc2c(N(c4ccc(-c5ccc(-c6ccc7ccccc7c6)cc5)cc4)c4ccc5ccccc5c4)cccc23)cc1 ; c1ccc(-c2ccc3c(c2)sc2c(N(c4ccc(-c5ccc6ccccc6c5)cc4)c4ccc5c(c4)oc4ccccc45)cccc23)cc1 ; c1ccc(-c2ccc3c(c2)sc2c(N(c4ccc(-c5ccc6c(ccc7ccccc76)c5)cc4)c4ccc5oc6ccccc6c5c4)cccc23)cc1]

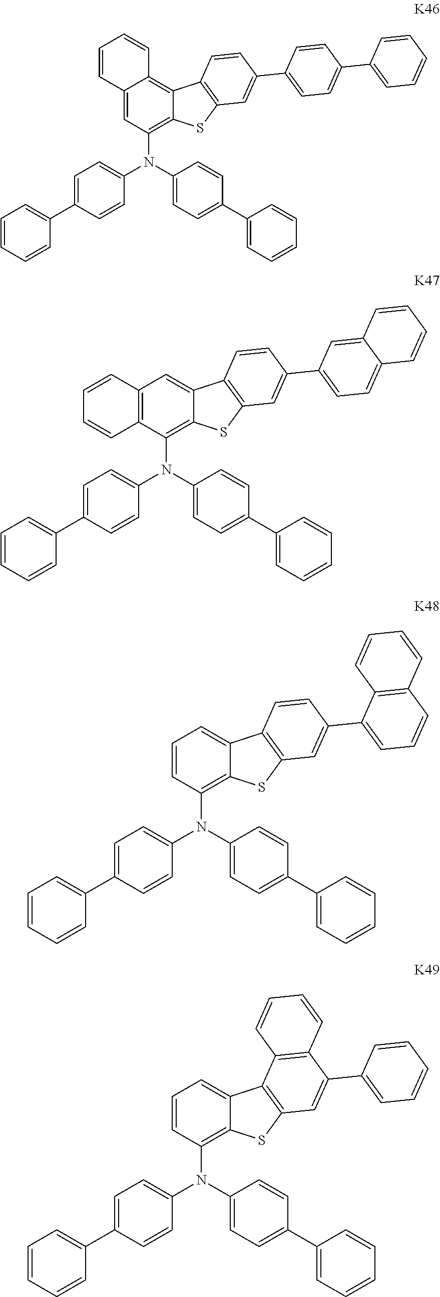 [SMILES: c1ccc(-c2ccc(-c3ccc4c(c3)sc3c(N(c5ccc(-c6ccccc6)cc5)c5ccc(-c6ccccc6)cc5)cc5ccccc5c34)cc2)cc1 ; c1ccc(-c2ccc(N(c3ccc(-c4ccccc4)cc3)c3c4ccccc4cc4c3sc3cc(-c5ccc6ccccc6c5)ccc34)cc2)cc1 ; c1ccc(-c2ccc(N(c3ccc(-c4ccccc4)cc3)c3cccc4c3sc3cc(-c5cccc6ccccc56)ccc34)cc2)cc1 ; c1ccc(-c2ccc(N(c3ccc(-c4ccccc4)cc3)c3cccc4c3sc3cc(-c5ccccc5)c5ccccc5c34)cc2)cc1]

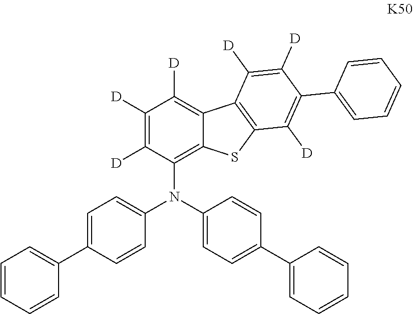 [SMILES: [2H]c1c([2H])c([2H])c2c(sc3c([2H])c(-c4ccccc4)c([2H])c([2H])c32)c1N(c1ccc(-c2ccccc2)cc1)c1ccc(-c2ccccc2)cc1]

14. The luminescence device of claim 1, wherein the amine compound represented by Formula 1 is any one selected from among compounds represented in Compound Group 3:

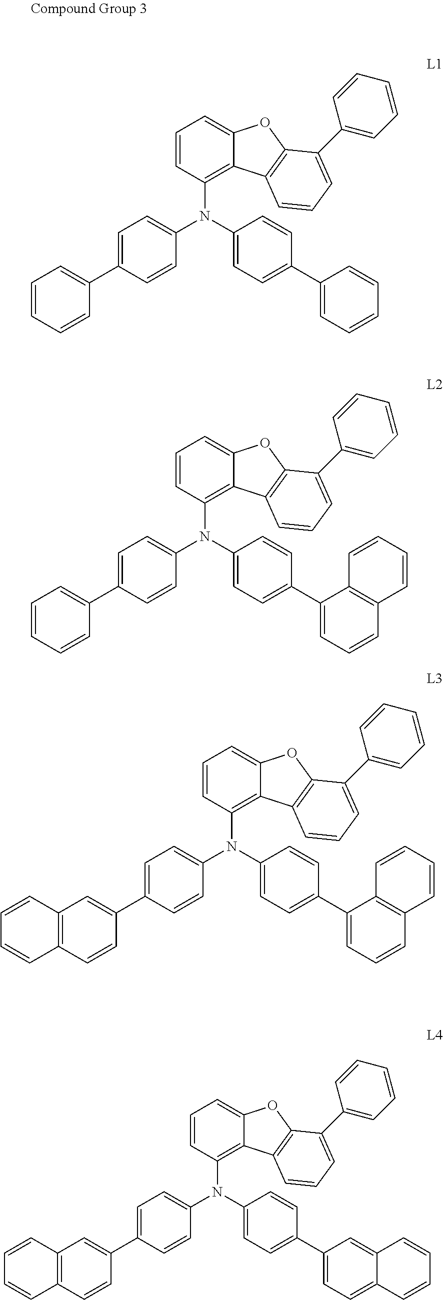 [SMILES: c1ccc(-c2ccc(N(c3ccc(-c4ccccc4)cc3)c3cccc4oc5c(-c6ccccc6)cccc5c34)cc2)cc1 ; c1ccc(-c2ccc(N(c3ccc(-c4cccc5ccccc45)cc3)c3cccc4oc5c(-c6ccccc6)cccc5c34)cc2)cc1 ; c1ccc(-c2cccc3c2oc2cccc(N(c4ccc(-c5ccc6ccccc6c5)cc4)c4ccc(-c5cccc6ccccc56)cc4)c23)cc1 ; c1ccc(-c2cccc3c2oc2cccc(N(c4ccc(-c5ccc6ccccc6c5)cc4)c4ccc(-c5ccc6ccccc6c5)cc4)c23)cc1]

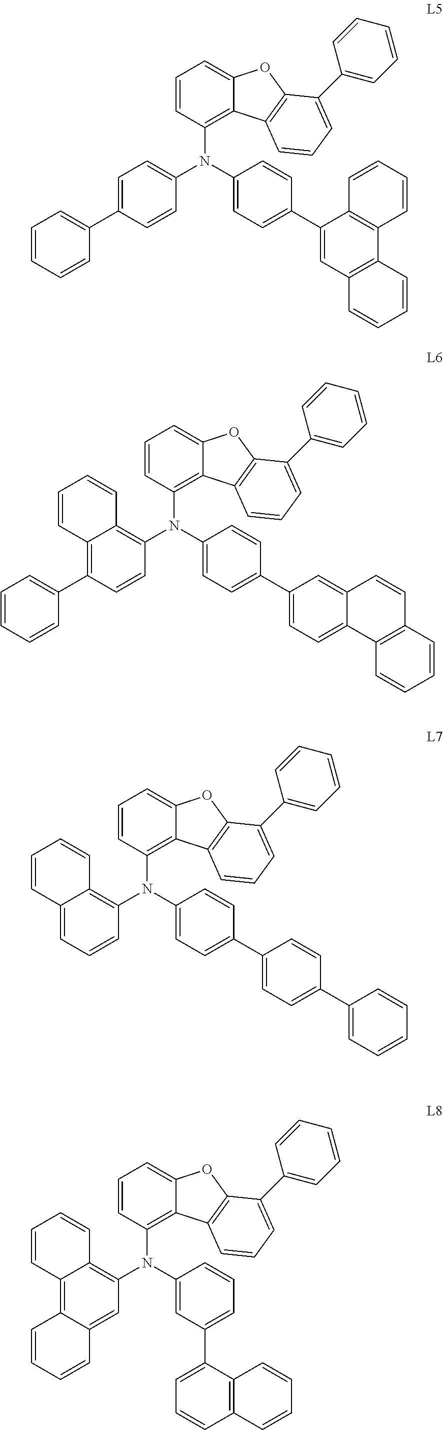 [SMILES: c1ccc(-c2ccc(N(c3ccc(-c4cc5ccccc5c5ccccc45)cc3)c3cccc4oc5c(-c6ccccc6)cccc5c34)cc2)cc1 ; c1ccc(-c2ccc(N(c3ccc(-c4ccc5c(ccc6ccccc65)c4)cc3)c3cccc4oc5c(-c6ccccc6)cccc5c34)c3ccccc23)cc1 ; c1ccc(-c2ccc(-c3ccc(N(c4cccc5ccccc45)c4cccc5oc6c(-c7ccccc7)cccc6c45)cc3)cc2)cc1 ; c1ccc(-c2cccc3c2oc2cccc(N(c4cccc(-c5cccc6ccccc56)c4)c4cc5ccccc5c5ccccc45)c23)cc1]

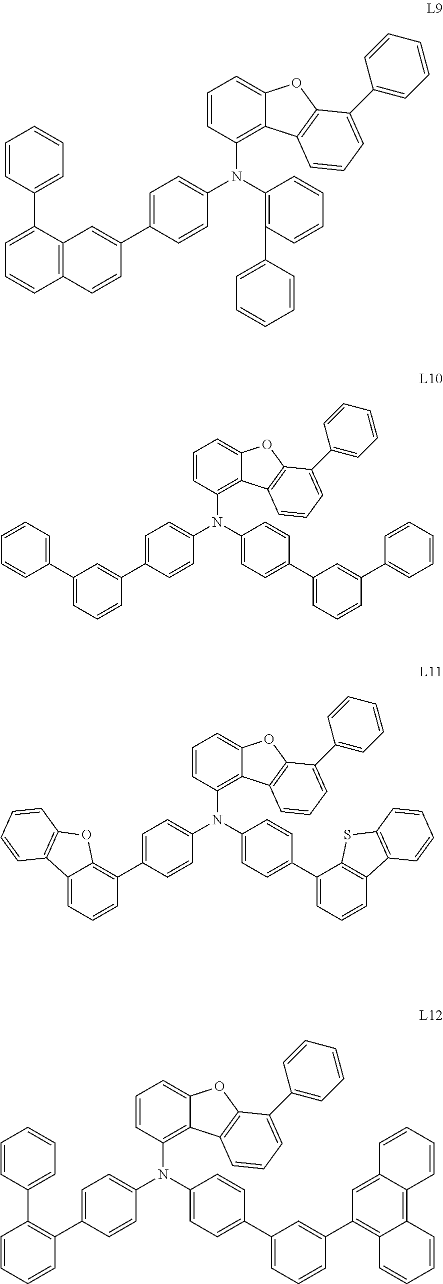 [SMILES: c1ccc(-c2ccccc2N(c2ccc(-c3ccc4cccc(-c5ccccc5)c4c3)cc2)c2cccc3oc4c(-c5ccccc5)cccc4c23)cc1 ; c1ccc(-c2cccc(-c3ccc(N(c4ccc(-c5cccc(-c6ccccc6)c5)cc4)c4cccc5oc6c(-c7ccccc7)cccc6c45)cc3)c2)cc1 ; c1ccc(-c2cccc3c2oc2cccc(N(c4ccc(-c5cccc6c5oc5ccccc56)cc4)c4ccc(-c5cccc6c5sc5ccccc56)cc4)c23)cc1 ; c1ccc(-c2ccccc2-c2ccc(N(c3ccc(-c4cccc(-c5cc6ccccc6c6ccccc56)c4)cc3)c3cccc4oc5c(-c6ccccc6)cccc5c34)cc2)cc1]

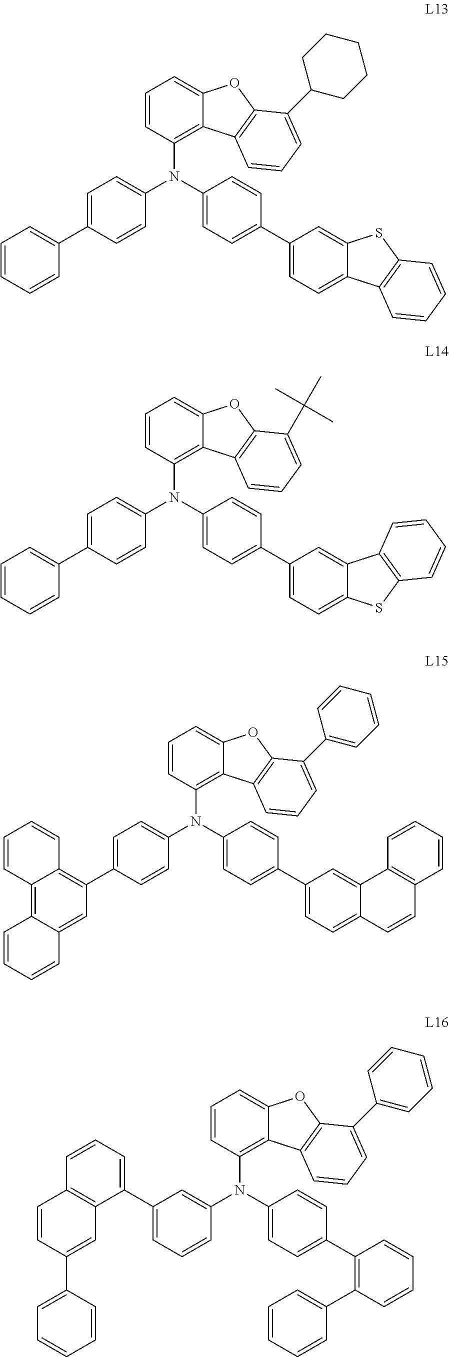 [SMILES: c1ccc(-c2ccc(N(c3ccc(-c4ccc5c(c4)sc4ccccc45)cc3)c3cccc4oc5c(C6CCCCC6)cccc5c34)cc2)cc1 ; CC(C)(C)c1cccc2c1oc1cccc(N(c3ccc(-c4ccccc4)cc3)c3ccc(-c4ccc5sc6ccccc6c5c4)cc3)c12 ; c1ccc(-c2cccc3c2oc2cccc(N(c4ccc(-c5ccc6ccc7ccccc7c6c5)cc4)c4ccc(-c5cc6ccccc6c6ccccc56)cc4)c23)cc1 ; c1ccc(-c2ccc3cccc(-c4cccc(N(c5ccc(-c6ccccc6-c6ccccc6)cc5)c5cccc6oc7c(-c8ccccc8)cccc7c56)c4)c3c2)cc1]

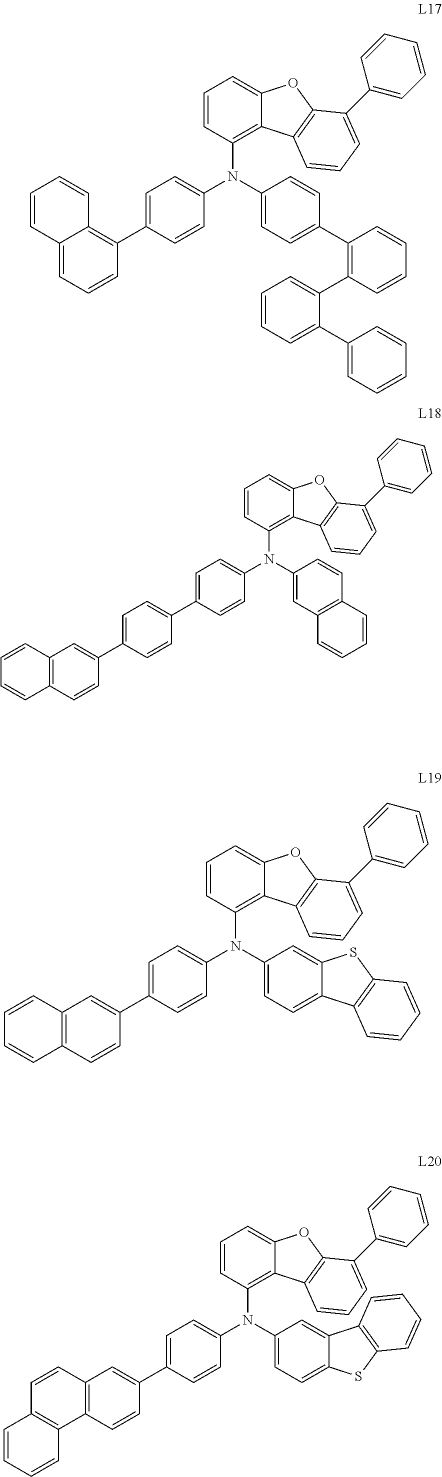 [SMILES: c1ccc(-c2ccccc2-c2ccccc2-c2ccc(N(c3ccc(-c4cccc5ccccc45)cc3)c3cccc4oc5c(-c6ccccc6)cccc5c34)cc2)cc1 ; c1ccc(-c2cccc3c2oc2cccc(N(c4ccc(-c5ccc(-c6ccc7ccccc7c6)cc5)cc4)c4ccc5ccccc5c4)c23)cc1 ; c1ccc(-c2cccc3c2oc2cccc(N(c4ccc(-c5ccc6ccccc6c5)cc4)c4ccc5c(c4)sc4ccccc45)c23)cc1 ; c1ccc(-c2cccc3c2oc2cccc(N(c4ccc(-c5ccc6c(ccc7ccccc76)c5)cc4)c4ccc5sc6ccccc6c5c4)c23)cc1]

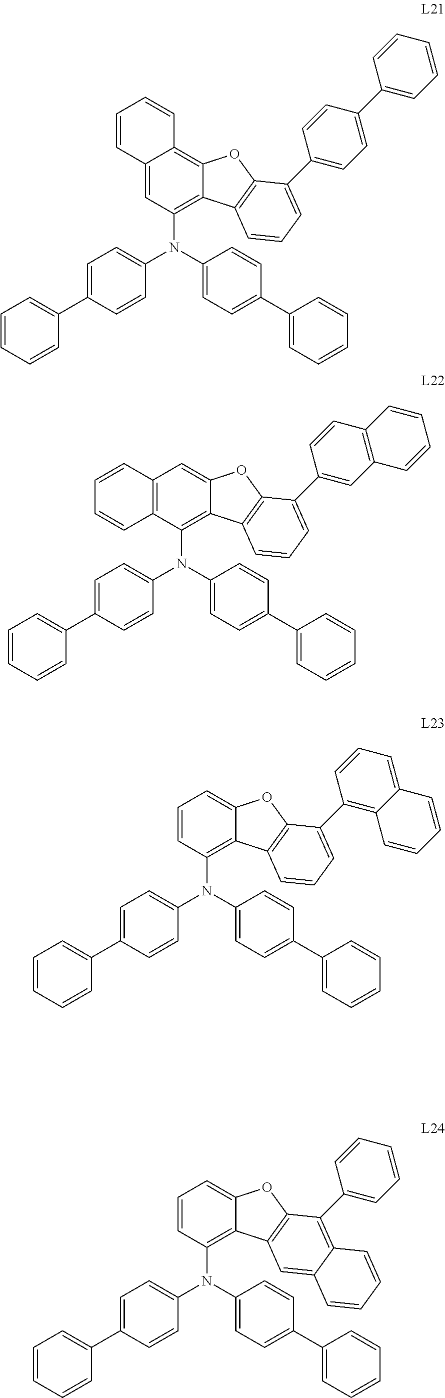 [SMILES: c1ccc(-c2ccc(-c3cccc4c3oc3c5ccccc5cc(N(c5ccc(-c6ccccc6)cc5)c5ccc(-c6ccccc6)cc5)c43)cc2)cc1 ; c1ccc(-c2ccc(N(c3ccc(-c4ccccc4)cc3)c3c4ccccc4cc4oc5c(-c6ccc7ccccc7c6)cccc5c34)cc2)cc1 ; c1ccc(-c2ccc(N(c3ccc(-c4ccccc4)cc3)c3cccc4oc5c(-c6cccc7ccccc67)cccc5c34)cc2)cc1 ; c1ccc(-c2ccc(N(c3ccc(-c4ccccc4)cc3)c3cccc4oc5c(-c6ccccc6)c6ccccc6cc5c34)cc2)cc1]

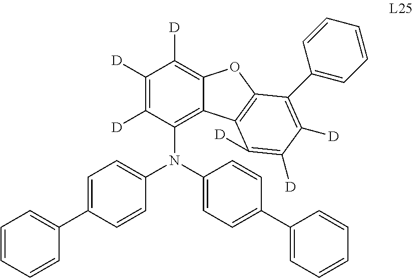 [SMILES: [2H]c1c([2H])c(N(c2ccc(-c3ccccc3)cc2)c2ccc(-c3ccccc3)cc2)c2c(oc3c(-c4ccccc4)c([2H])c([2H])c([2H])c32)c1[2H]]

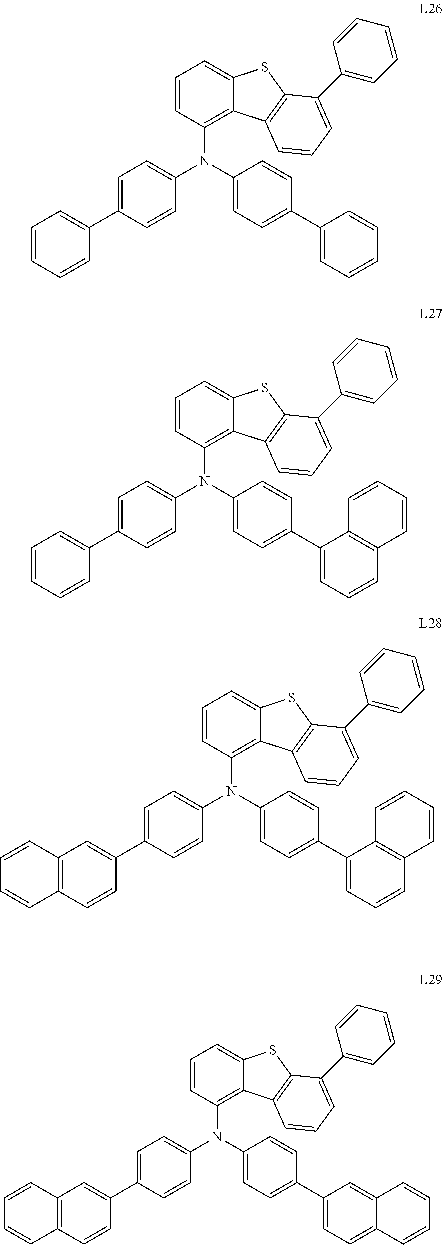 [SMILES: c1ccc(-c2ccc(N(c3ccc(-c4ccccc4)cc3)c3cccc4sc5c(-c6ccccc6)cccc5c34)cc2)cc1 ; c1ccc(-c2ccc(N(c3ccc(-c4cccc5ccccc45)cc3)c3cccc4sc5c(-c6ccccc6)cccc5c34)cc2)cc1 ; c1ccc(-c2cccc3c2sc2cccc(N(c4ccc(-c5ccc6ccccc6c5)cc4)c4ccc(-c5cccc6ccccc56)cc4)c23)cc1 ; c1ccc(-c2cccc3c2sc2cccc(N(c4ccc(-c5ccc6ccccc6c5)cc4)c4ccc(-c5ccc6ccccc6c5)cc4)c23)cc1]

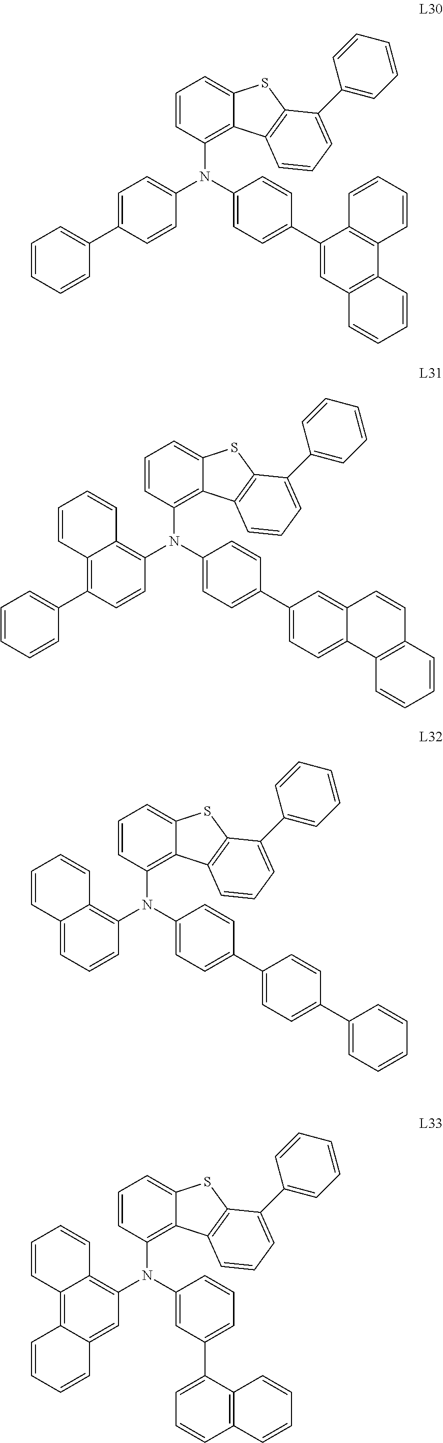 [SMILES: c1ccc(-c2ccc(N(c3ccc(-c4cc5ccccc5c5ccccc45)cc3)c3cccc4sc5c(-c6ccccc6)cccc5c34)cc2)cc1 ; c1ccc(-c2ccc(N(c3ccc(-c4ccc5c(ccc6ccccc65)c4)cc3)c3cccc4sc5c(-c6ccccc6)cccc5c34)c3ccccc23)cc1 ; c1ccc(-c2ccc(-c3ccc(N(c4cccc5ccccc45)c4cccc5sc6c(-c7ccccc7)cccc6c45)cc3)cc2)cc1 ; c1ccc(-c2cccc3c2sc2cccc(N(c4cccc(-c5cccc6ccccc56)c4)c4cc5ccccc5c5ccccc45)c23)cc1]

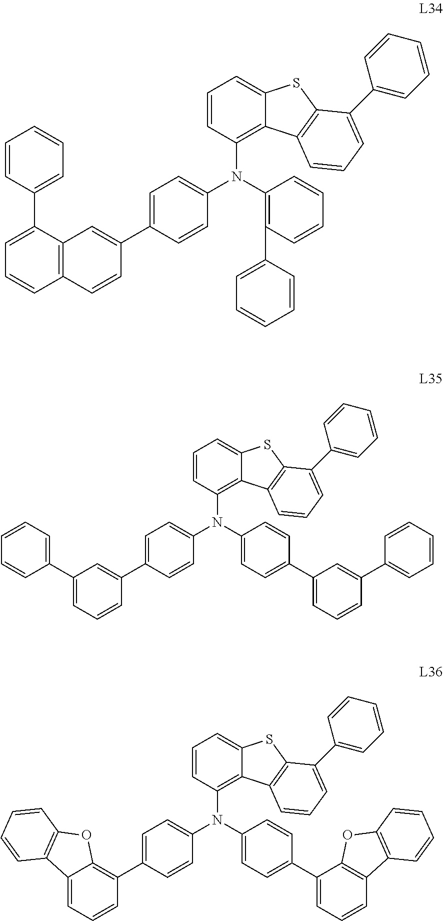 [SMILES: c1ccc(-c2ccccc2N(c2ccc(-c3ccc4cccc(-c5ccccc5)c4c3)cc2)c2cccc3sc4c(-c5ccccc5)cccc4c23)cc1 ; c1ccc(-c2cccc(-c3ccc(N(c4ccc(-c5cccc(-c6ccccc6)c5)cc4)c4cccc5sc6c(-c7ccccc7)cccc6c45)cc3)c2)cc1 ; c1ccc(-c2cccc3c2sc2cccc(N(c4ccc(-c5cccc6c5oc5ccccc56)cc4)c4ccc(-c5cccc6c5oc5ccccc56)cc4)c23)cc1]

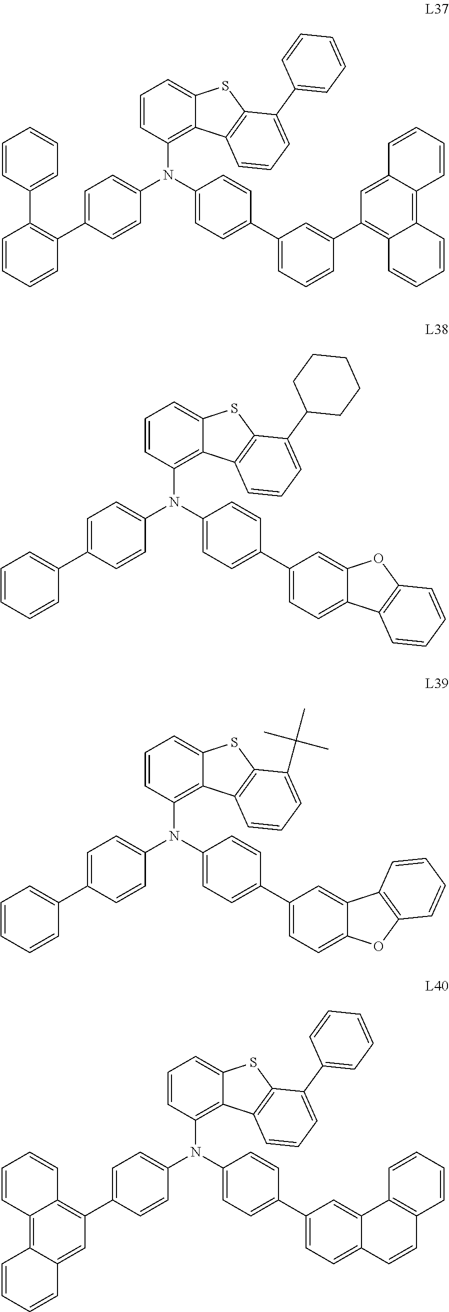 [SMILES: c1ccc(-c2ccccc2-c2ccc(N(c3ccc(-c4cccc(-c5cc6ccccc6c6ccccc56)c4)cc3)c3cccc4sc5c(-c6ccccc6)cccc5c34)cc2)cc1 ; c1ccc(-c2ccc(N(c3ccc(-c4ccc5c(c4)oc4ccccc45)cc3)c3cccc4sc5c(C6CCCCC6)cccc5c34)cc2)cc1 ; CC(C)(C)c1cccc2c1sc1cccc(N(c3ccc(-c4ccccc4)cc3)c3ccc(-c4ccc5oc6ccccc6c5c4)cc3)c12 ; c1ccc(-c2cccc3c2sc2cccc(N(c4ccc(-c5ccc6ccc7ccccc7c6c5)cc4)c4ccc(-c5cc6ccccc6c6ccccc56)cc4)c23)cc1]

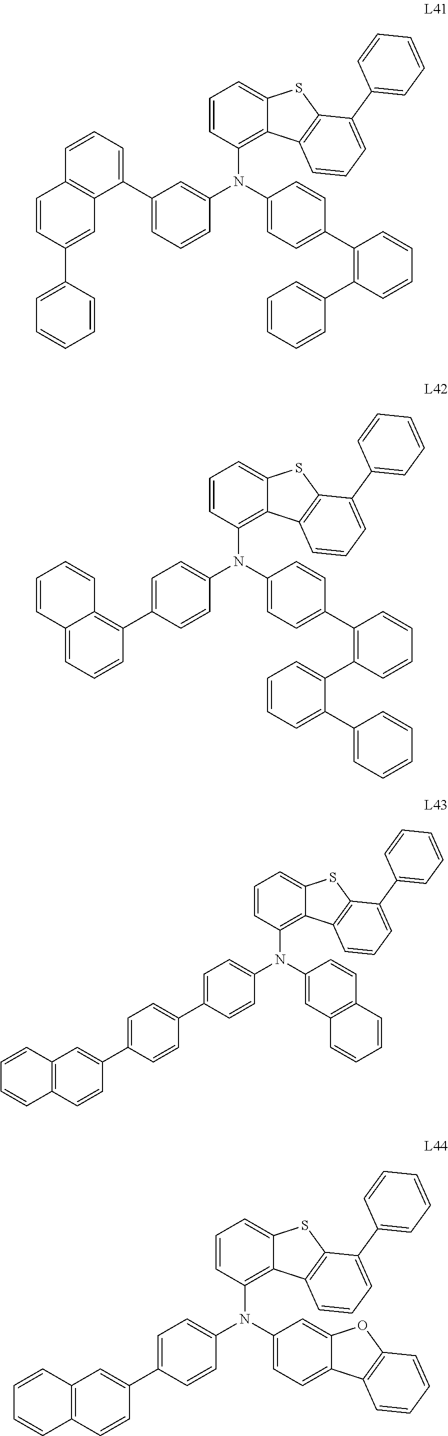 [SMILES: c1ccc(-c2ccc3cccc(-c4cccc(N(c5ccc(-c6ccccc6-c6ccccc6)cc5)c5cccc6sc7c(-c8ccccc8)cccc7c56)c4)c3c2)cc1 ; c1ccc(-c2ccccc2-c2ccccc2-c2ccc(N(c3ccc(-c4cccc5ccccc45)cc3)c3cccc4sc5c(-c6ccccc6)cccc5c34)cc2)cc1 ; c1ccc(-c2cccc3c2sc2cccc(N(c4ccc(-c5ccc(-c6ccc7ccccc7c6)cc5)cc4)c4ccc5ccccc5c4)c23)cc1 ; c1ccc(-c2cccc3c2sc2cccc(N(c4ccc(-c5ccc6ccccc6c5)cc4)c4ccc5c(c4)oc4ccccc45)c23)cc1]

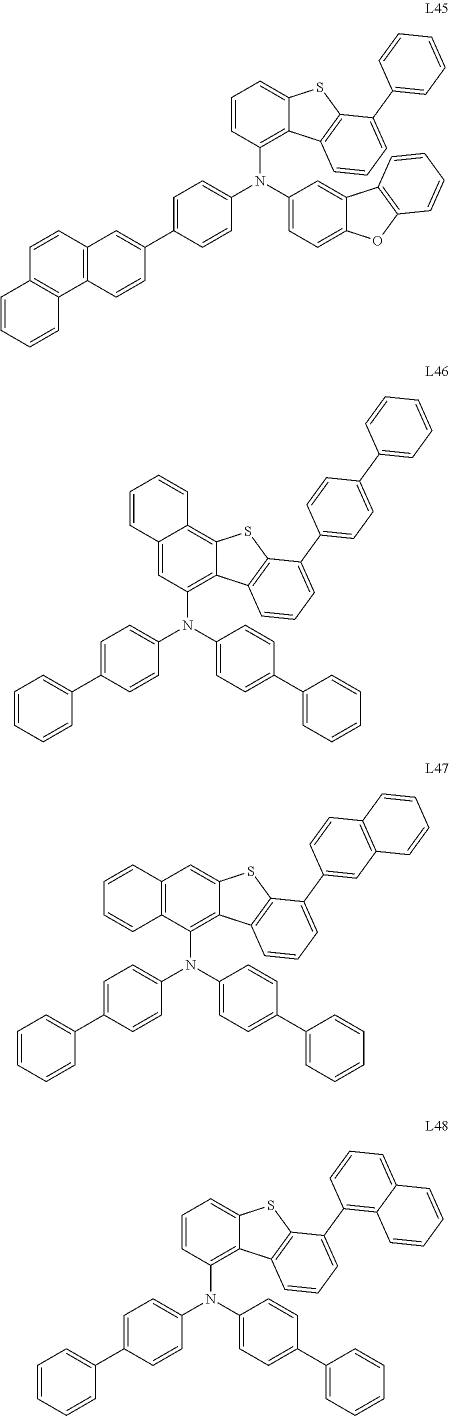 [SMILES: c1ccc(-c2cccc3c2sc2cccc(N(c4ccc(-c5ccc6c(ccc7ccccc76)c5)cc4)c4ccc5oc6ccccc6c5c4)c23)cc1 ; c1ccc(-c2ccc(-c3cccc4c3sc3c5ccccc5cc(N(c5ccc(-c6ccccc6)cc5)c5ccc(-c6ccccc6)cc5)c43)cc2)cc1 ; c1ccc(-c2ccc(N(c3ccc(-c4ccccc4)cc3)c3c4ccccc4cc4sc5c(-c6ccc7ccccc7c6)cccc5c34)cc2)cc1 ; c1ccc(-c2ccc(N(c3ccc(-c4ccccc4)cc3)c3cccc4sc5c(-c6cccc7ccccc67)cccc5c34)cc2)cc1]

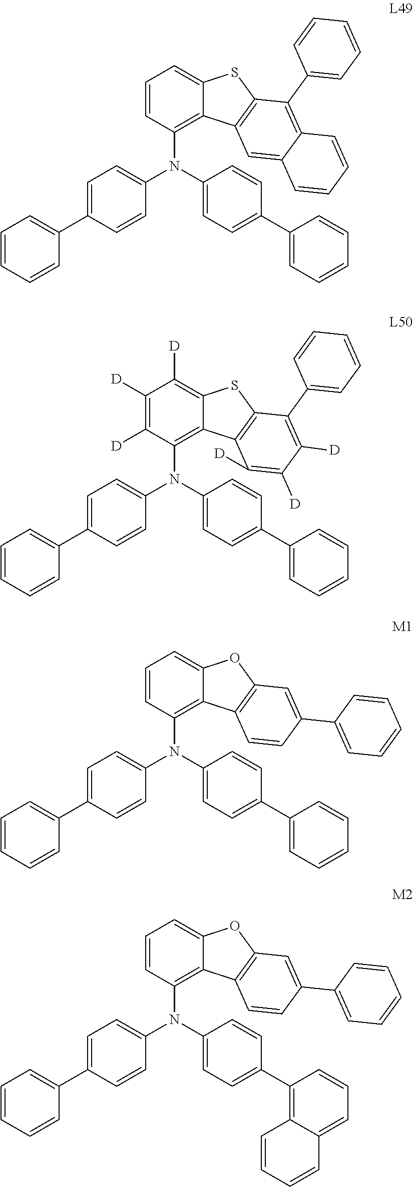 [SMILES: c1ccc(-c2ccc(N(c3ccc(-c4ccccc4)cc3)c3cccc4sc5c(-c6ccccc6)c6ccccc6cc5c34)cc2)cc1 ; [2H]c1c([2H])c(N(c2ccc(-c3ccccc3)cc2)c2ccc(-c3ccccc3)cc2)c2c(sc3c(-c4ccccc4)c([2H])c([2H])c([2H])c32)c1[2H] ; c1ccc(-c2ccc(N(c3ccc(-c4ccccc4)cc3)c3cccc4oc5cc(-c6ccccc6)ccc5c34)cc2)cc1 ; c1ccc(-c2ccc(N(c3ccc(-c4cccc5ccccc45)cc3)c3cccc4oc5cc(-c6ccccc6)ccc5c34)cc2)cc1]

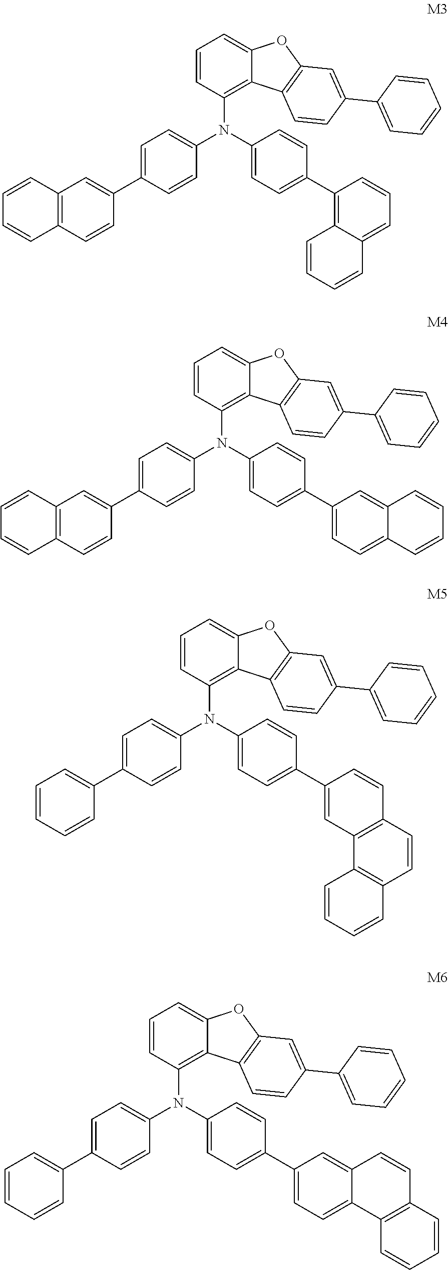 [SMILES: c1ccc(-c2ccc3c(c2)oc2cccc(N(c4ccc(-c5ccc6ccccc6c5)cc4)c4ccc(-c5cccc6ccccc56)cc4)c23)cc1 ; c1ccc(-c2ccc3c(c2)oc2cccc(N(c4ccc(-c5ccc6ccccc6c5)cc4)c4ccc(-c5ccc6ccccc6c5)cc4)c23)cc1 ; c1ccc(-c2ccc(N(c3ccc(-c4ccc5ccc6ccccc6c5c4)cc3)c3cccc4oc5cc(-c6ccccc6)ccc5c34)cc2)cc1 ; c1ccc(-c2ccc(N(c3ccc(-c4ccc5c(ccc6ccccc65)c4)cc3)c3cccc4oc5cc(-c6ccccc6)ccc5c34)cc2)cc1]

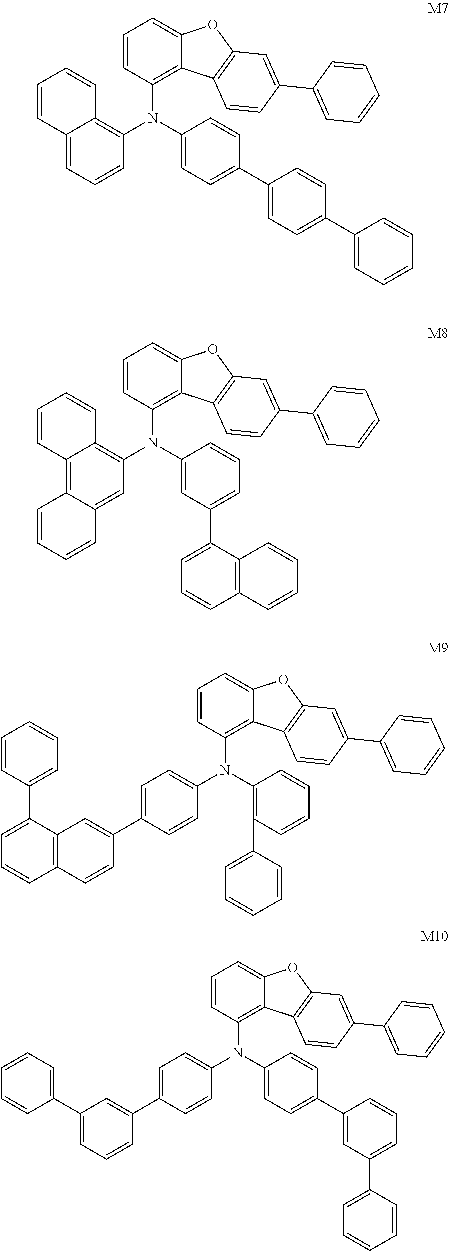 [SMILES: c1ccc(-c2ccc(-c3ccc(N(c4cccc5ccccc45)c4cccc5oc6cc(-c7ccccc7)ccc6c45)cc3)cc2)cc1 ; c1ccc(-c2ccc3c(c2)oc2cccc(N(c4cccc(-c5cccc6ccccc56)c4)c4cc5ccccc5c5ccccc45)c23)cc1 ; c1ccc(-c2ccc3c(c2)oc2cccc(N(c4ccc(-c5ccc6cccc(-c7ccccc7)c6c5)cc4)c4ccccc4-c4ccccc4)c23)cc1 ; c1ccc(-c2cccc(-c3ccc(N(c4ccc(-c5cccc(-c6ccccc6)c5)cc4)c4cccc5oc6cc(-c7ccccc7)ccc6c45)cc3)c2)cc1]

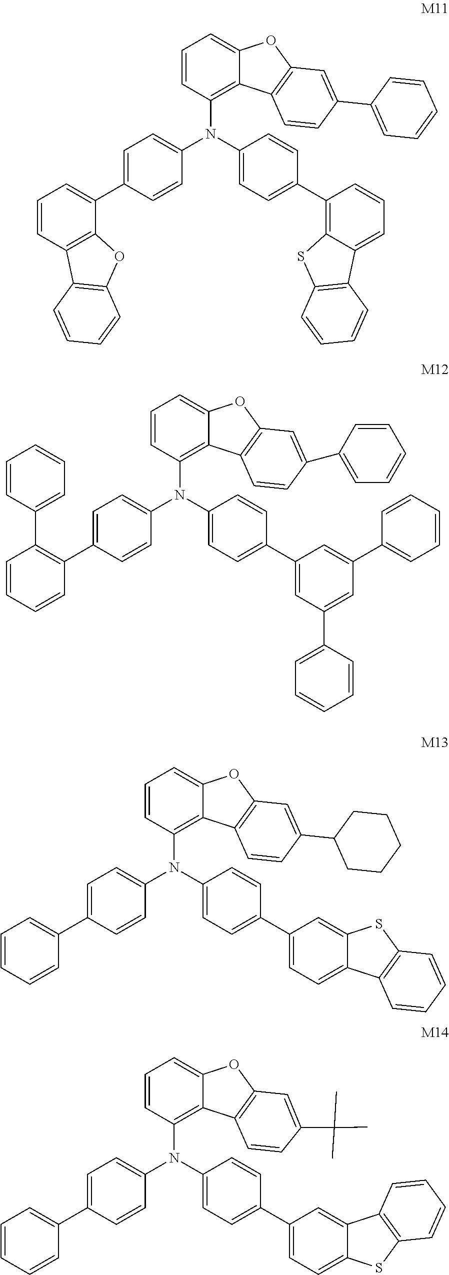 [SMILES: c1ccc(-c2ccc3c(c2)oc2cccc(N(c4ccc(-c5cccc6c5oc5ccccc56)cc4)c4ccc(-c5cccc6c5sc5ccccc56)cc4)c23)cc1 ; c1ccc(-c2cc(-c3ccccc3)cc(-c3ccc(N(c4ccc(-c5ccccc5-c5ccccc5)cc4)c4cccc5oc6cc(-c7ccccc7)ccc6c45)cc3)c2)cc1 ; c1ccc(-c2ccc(N(c3ccc(-c4ccc5c(c4)sc4ccccc45)cc3)c3cccc4oc5cc(C6CCCCC6)ccc5c34)cc2)cc1 ; CC(C)(C)c1ccc2c(c1)oc1cccc(N(c3ccc(-c4ccccc4)cc3)c3ccc(-c4ccc5sc6ccccc6c5c4)cc3)c12]

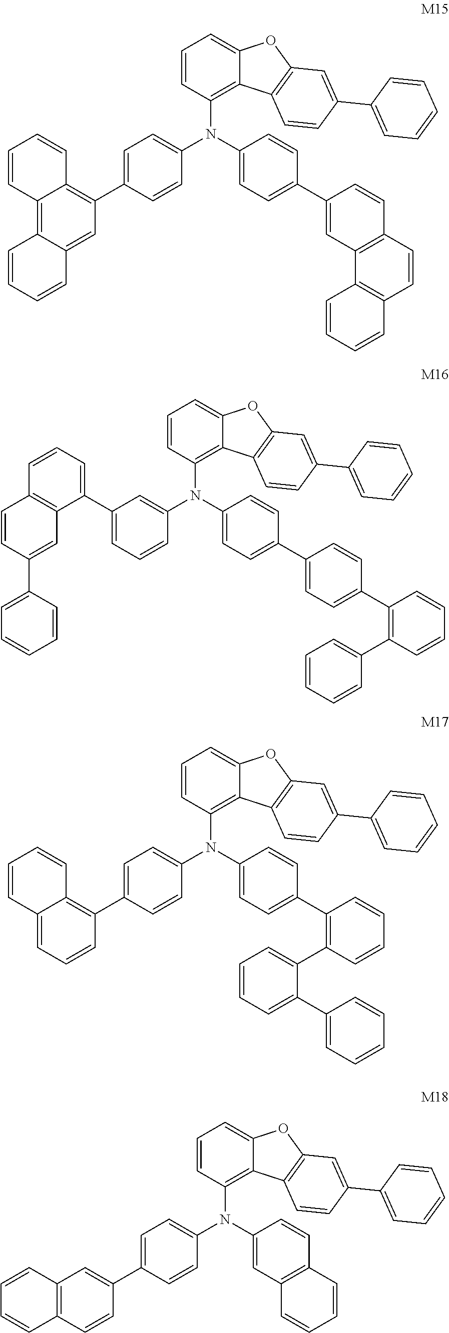 [SMILES: c1ccc(-c2ccc3c(c2)oc2cccc(N(c4ccc(-c5ccc6ccc7ccccc7c6c5)cc4)c4ccc(-c5cc6ccccc6c6ccccc56)cc4)c23)cc1 ; c1ccc(-c2ccc3c(c2)oc2cccc(N(c4ccc(-c5ccc(-c6ccccc6-c6ccccc6)cc5)cc4)c4cccc(-c5cccc6ccc(-c7ccccc7)cc56)c4)c23)cc1 ; c1ccc(-c2ccc3c(c2)oc2cccc(N(c4ccc(-c5ccccc5-c5ccccc5-c5ccccc5)cc4)c4ccc(-c5cccc6ccccc56)cc4)c23)cc1 ; c1ccc(-c2ccc3c(c2)oc2cccc(N(c4ccc(-c5ccc6ccccc6c5)cc4)c4ccc5ccccc5c4)c23)cc1]

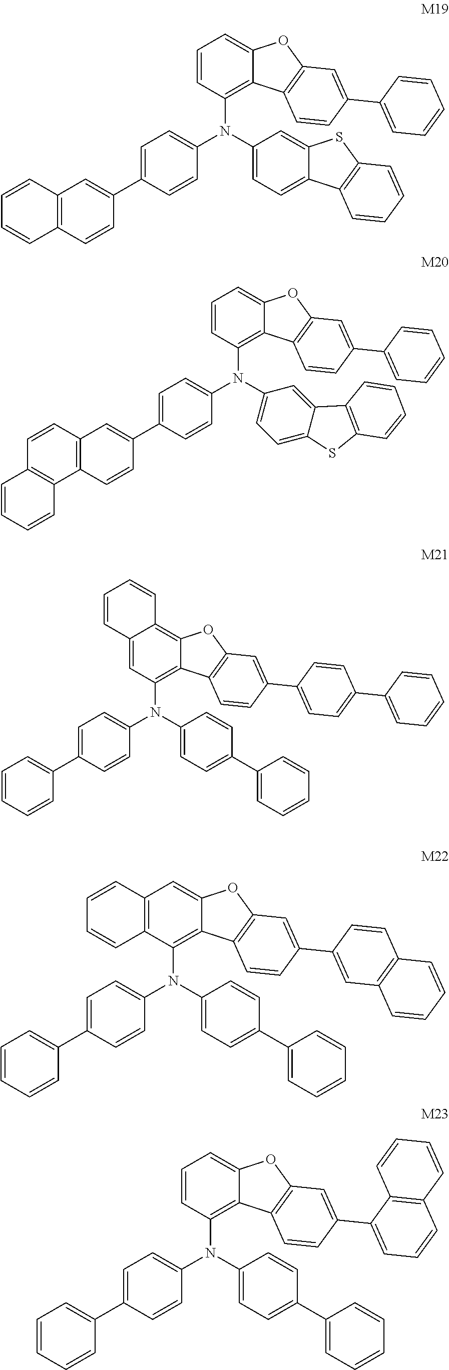 [SMILES: c1ccc(-c2ccc3c(c2)oc2cccc(N(c4ccc(-c5ccc6ccccc6c5)cc4)c4ccc5c(c4)sc4ccccc45)c23)cc1 ; c1ccc(-c2ccc3c(c2)oc2cccc(N(c4ccc(-c5ccc6c(ccc7ccccc76)c5)cc4)c4ccc5sc6ccccc6c5c4)c23)cc1 ; c1ccc(-c2ccc(-c3ccc4c(c3)oc3c5ccccc5cc(N(c5ccc(-c6ccccc6)cc5)c5ccc(-c6ccccc6)cc5)c43)cc2)cc1 ; c1ccc(-c2ccc(N(c3ccc(-c4ccccc4)cc3)c3c4ccccc4cc4oc5cc(-c6ccc7ccccc7c6)ccc5c34)cc2)cc1 ; c1ccc(-c2ccc(N(c3ccc(-c4ccccc4)cc3)c3cccc4oc5cc(-c6cccc7ccccc67)ccc5c34)cc2)cc1]

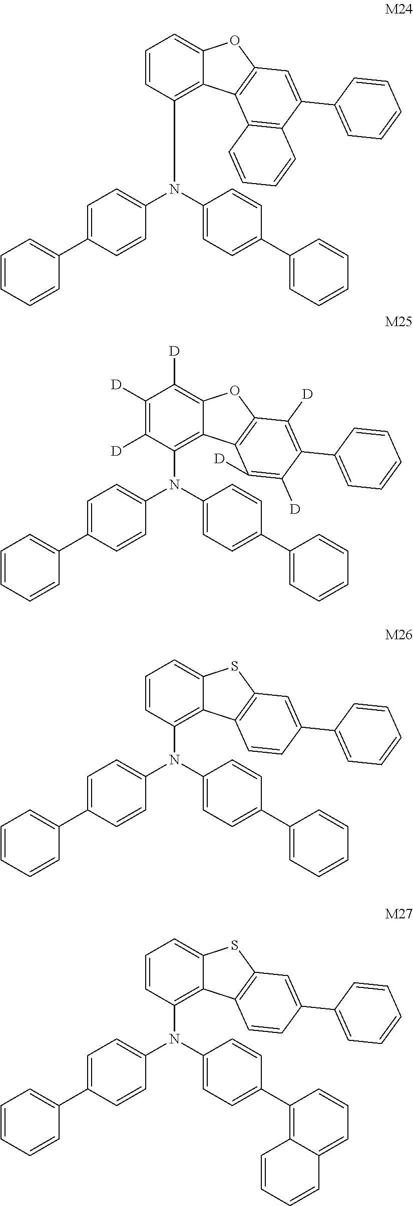 [SMILES: c1ccc(-c2ccc(N(c3ccc(-c4ccccc4)cc3)c3cccc4oc5cc(-c6ccccc6)c6ccccc6c5c34)cc2)cc1 ; [2H]c1c([2H])c(N(c2ccc(-c3ccccc3)cc2)c2ccc(-c3ccccc3)cc2)c2c(oc3c([2H])c(-c4ccccc4)c([2H])c([2H])c32)c1[2H] ; c1ccc(-c2ccc(N(c3ccc(-c4ccccc4)cc3)c3cccc4sc5cc(-c6ccccc6)ccc5c34)cc2)cc1 ; c1ccc(-c2ccc(N(c3ccc(-c4cccc5ccccc45)cc3)c3cccc4sc5cc(-c6ccccc6)ccc5c34)cc2)cc1]

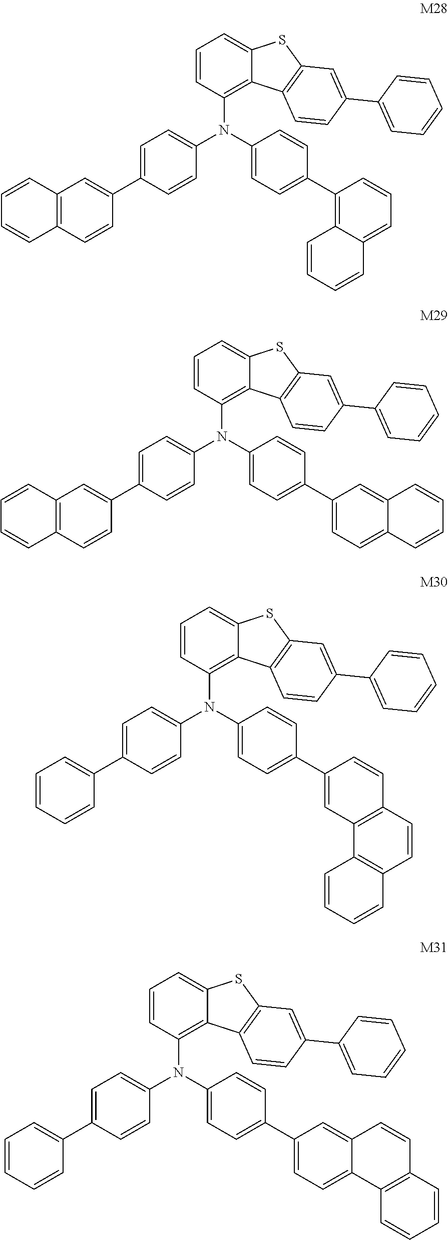 [SMILES: c1ccc(-c2ccc3c(c2)sc2cccc(N(c4ccc(-c5ccc6ccccc6c5)cc4)c4ccc(-c5cccc6ccccc56)cc4)c23)cc1 ; c1ccc(-c2ccc3c(c2)sc2cccc(N(c4ccc(-c5ccc6ccccc6c5)cc4)c4ccc(-c5ccc6ccccc6c5)cc4)c23)cc1 ; c1ccc(-c2ccc(N(c3ccc(-c4ccc5ccc6ccccc6c5c4)cc3)c3cccc4sc5cc(-c6ccccc6)ccc5c34)cc2)cc1 ; c1ccc(-c2ccc(N(c3ccc(-c4ccc5c(ccc6ccccc65)c4)cc3)c3cccc4sc5cc(-c6ccccc6)ccc5c34)cc2)cc1]

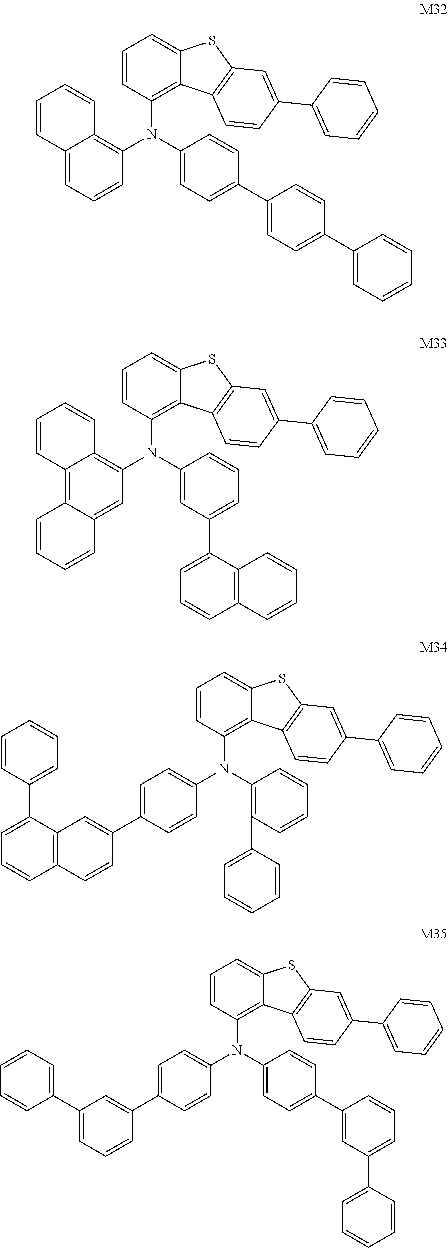 [SMILES: c1ccc(-c2ccc(-c3ccc(N(c4cccc5ccccc45)c4cccc5sc6cc(-c7ccccc7)ccc6c45)cc3)cc2)cc1 ; c1ccc(-c2ccc3c(c2)sc2cccc(N(c4cccc(-c5cccc6ccccc56)c4)c4cc5ccccc5c5ccccc45)c23)cc1 ; c1ccc(-c2ccc3c(c2)sc2cccc(N(c4ccc(-c5ccc6cccc(-c7ccccc7)c6c5)cc4)c4ccccc4-c4ccccc4)c23)cc1 ; c1ccc(-c2cccc(-c3ccc(N(c4ccc(-c5cccc(-c6ccccc6)c5)cc4)c4cccc5sc6cc(-c7ccccc7)ccc6c45)cc3)c2)cc1]

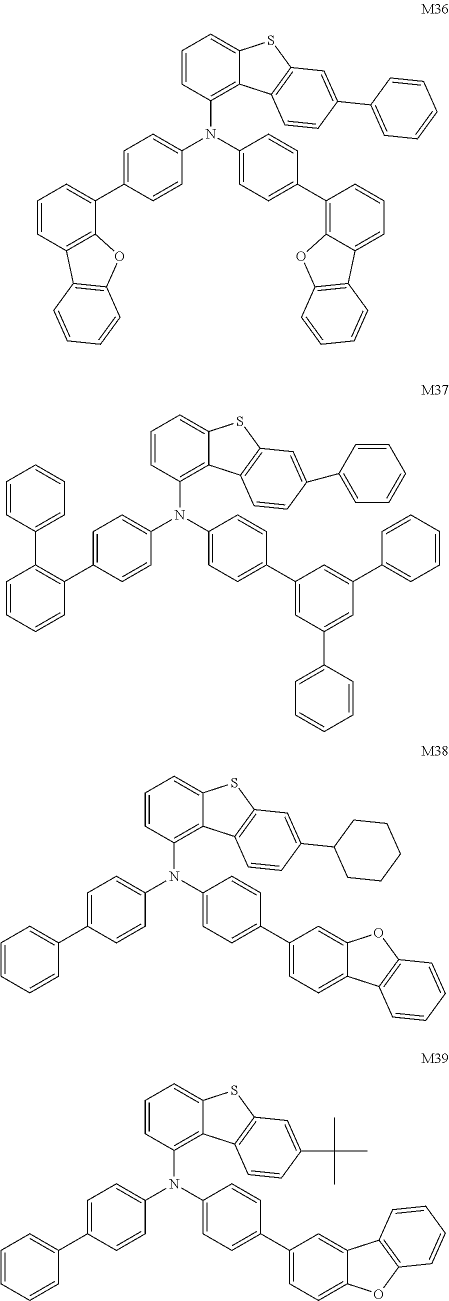 [SMILES: c1ccc(-c2ccc3c(c2)sc2cccc(N(c4ccc(-c5cccc6c5oc5ccccc56)cc4)c4ccc(-c5cccc6c5oc5ccccc56)cc4)c23)cc1 ; c1ccc(-c2cc(-c3ccccc3)cc(-c3ccc(N(c4ccc(-c5ccccc5-c5ccccc5)cc4)c4cccc5sc6cc(-c7ccccc7)ccc6c45)cc3)c2)cc1 ; c1ccc(-c2ccc(N(c3ccc(-c4ccc5c(c4)oc4ccccc45)cc3)c3cccc4sc5cc(C6CCCCC6)ccc5c34)cc2)cc1 ; CC(C)(C)c1ccc2c(c1)sc1cccc(N(c3ccc(-c4ccccc4)cc3)c3ccc(-c4ccc5oc6ccccc6c5c4)cc3)c12]

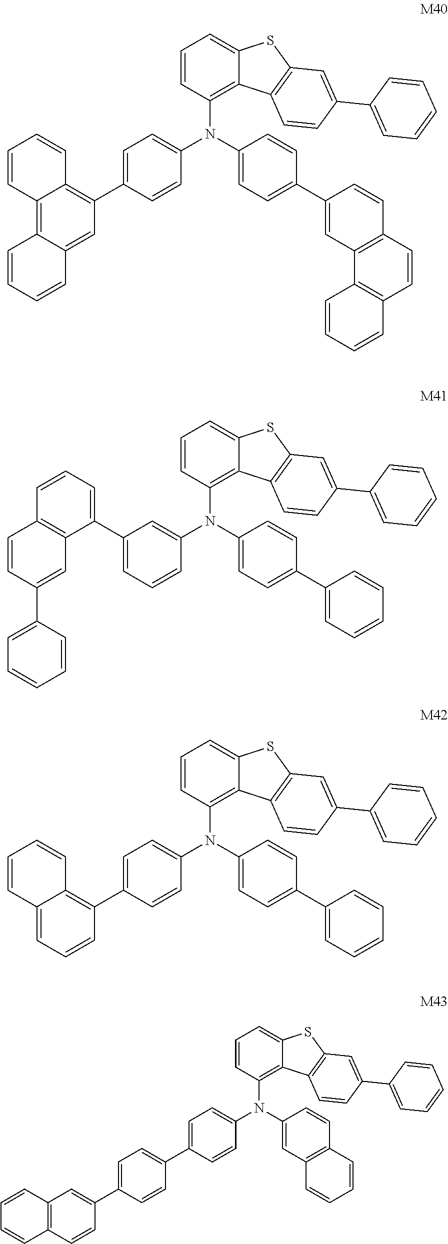 [SMILES: c1ccc(-c2ccc3c(c2)sc2cccc(N(c4ccc(-c5ccc6ccc7ccccc7c6c5)cc4)c4ccc(-c5cc6ccccc6c6ccccc56)cc4)c23)cc1 ; c1ccc(-c2ccc(N(c3cccc(-c4cccc5ccc(-c6ccccc6)cc45)c3)c3cccc4sc5cc(-c6ccccc6)ccc5c34)cc2)cc1 ; c1ccc(-c2ccc(N(c3ccc(-c4cccc5ccccc45)cc3)c3cccc4sc5cc(-c6ccccc6)ccc5c34)cc2)cc1 ; c1ccc(-c2ccc3c(c2)sc2cccc(N(c4ccc(-c5ccc(-c6ccc7ccccc7c6)cc5)cc4)c4ccc5ccccc5c4)c23)cc1]

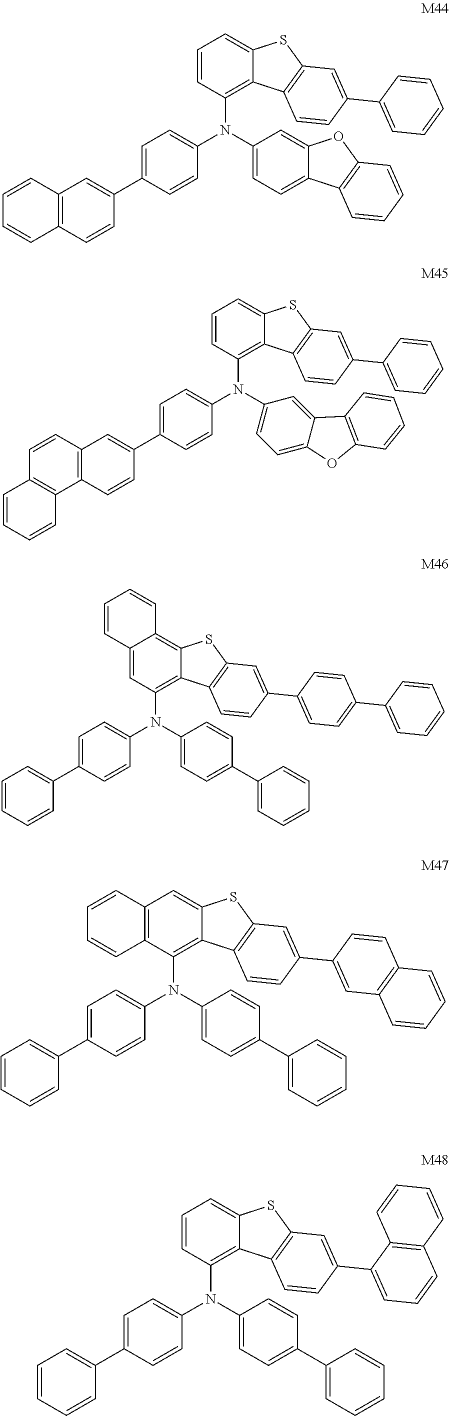 [SMILES: c1ccc(-c2ccc3c(c2)sc2cccc(N(c4ccc(-c5ccc6ccccc6c5)cc4)c4ccc5c(c4)oc4ccccc45)c23)cc1 ; c1ccc(-c2ccc3c(c2)sc2cccc(N(c4ccc(-c5ccc6c(ccc7ccccc76)c5)cc4)c4ccc5oc6ccccc6c5c4)c23)cc1 ; c1ccc(-c2ccc(-c3ccc4c(c3)sc3c5ccccc5cc(N(c5ccc(-c6ccccc6)cc5)c5ccc(-c6ccccc6)cc5)c43)cc2)cc1 ; c1ccc(-c2ccc(N(c3ccc(-c4ccccc4)cc3)c3c4ccccc4cc4sc5cc(-c6ccc7ccccc7c6)ccc5c34)cc2)cc1 ; c1ccc(-c2ccc(N(c3ccc(-c4ccccc4)cc3)c3cccc4sc5cc(-c6cccc7ccccc67)ccc5c34)cc2)cc1]

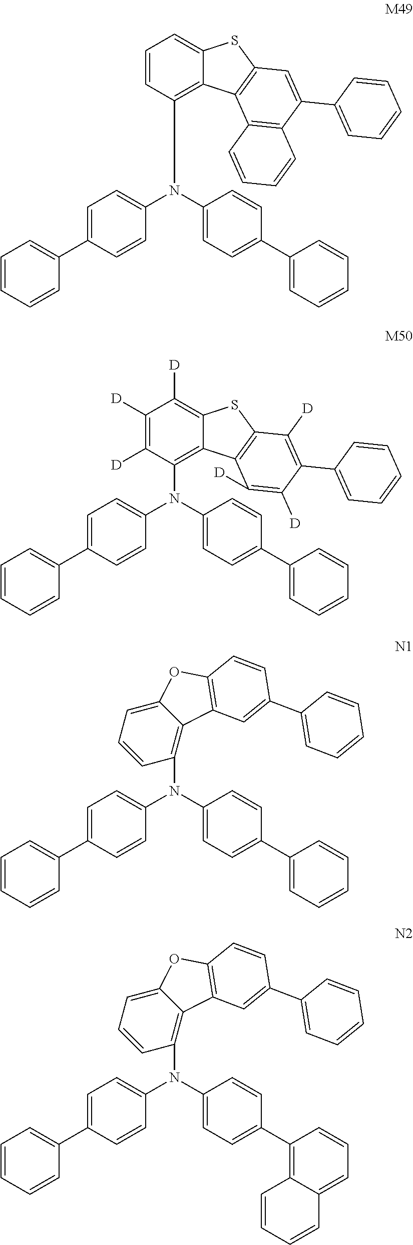 [SMILES: c1ccc(-c2ccc(N(c3ccc(-c4ccccc4)cc3)c3cccc4sc5cc(-c6ccccc6)c6ccccc6c5c34)cc2)cc1 ; [2H]c1c([2H])c(N(c2ccc(-c3ccccc3)cc2)c2ccc(-c3ccccc3)cc2)c2c(sc3c([2H])c(-c4ccccc4)c([2H])c([2H])c32)c1[2H] ; c1ccc(-c2ccc(N(c3ccc(-c4ccccc4)cc3)c3cccc4oc5ccc(-c6ccccc6)cc5c34)cc2)cc1 ; c1ccc(-c2ccc(N(c3ccc(-c4cccc5ccccc45)cc3)c3cccc4oc5ccc(-c6ccccc6)cc5c34)cc2)cc1]

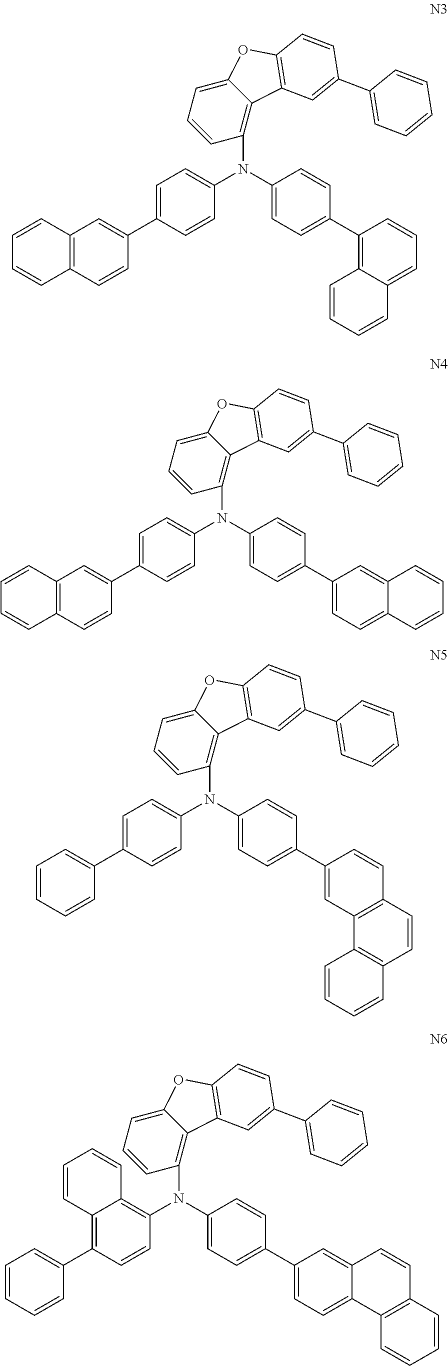 [SMILES: c1ccc(-c2ccc3oc4cccc(N(c5ccc(-c6ccc7ccccc7c6)cc5)c5ccc(-c6cccc7ccccc67)cc5)c4c3c2)cc1 ; c1ccc(-c2ccc3oc4cccc(N(c5ccc(-c6ccc7ccccc7c6)cc5)c5ccc(-c6ccc7ccccc7c6)cc5)c4c3c2)cc1 ; c1ccc(-c2ccc(N(c3ccc(-c4ccc5ccc6ccccc6c5c4)cc3)c3cccc4oc5ccc(-c6ccccc6)cc5c34)cc2)cc1 ; c1ccc(-c2ccc3oc4cccc(N(c5ccc(-c6ccc7c(ccc8ccccc87)c6)cc5)c5ccc(-c6ccccc6)c6ccccc56)c4c3c2)cc1]

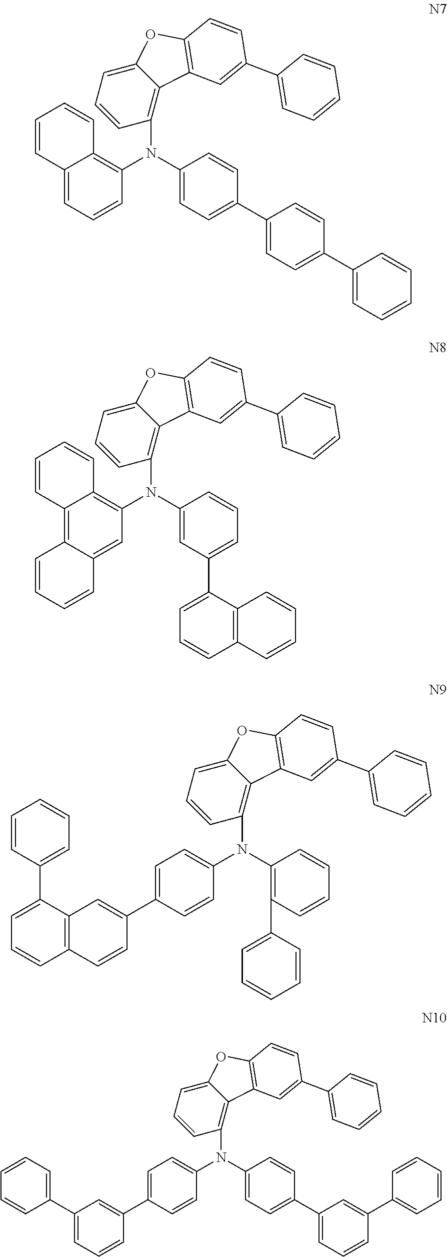

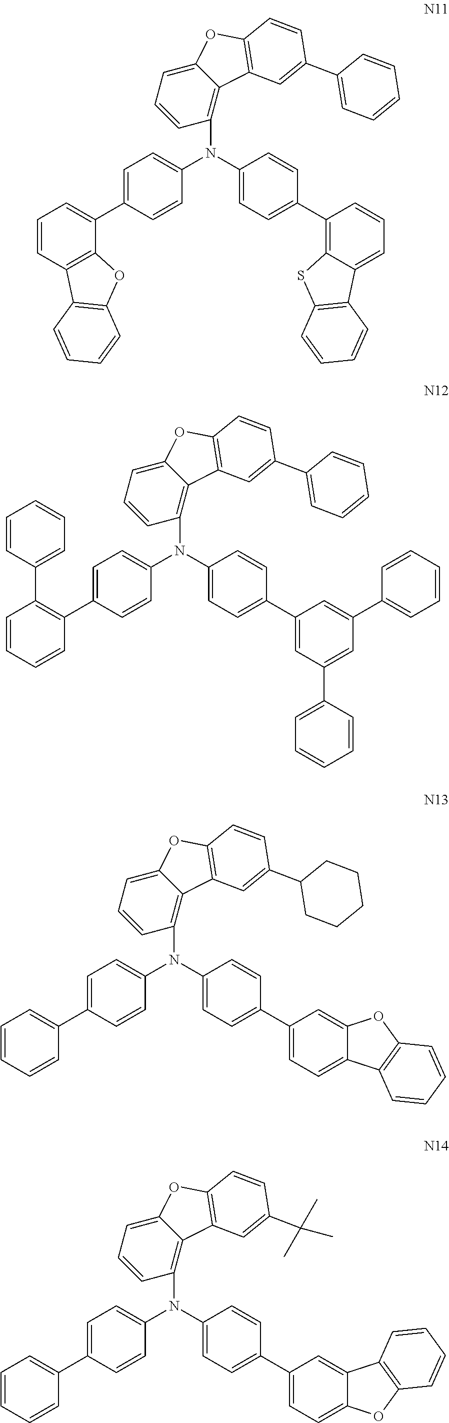 [SMILES: c1ccc(-c2ccc3oc4cccc(N(c5ccc(-c6cccc7c6oc6ccccc67)cc5)c5ccc(-c6cccc7c6sc6ccccc67)cc5)c4c3c2)cc1 ; c1ccc(-c2cc(-c3ccccc3)cc(-c3ccc(N(c4ccc(-c5ccccc5-c5ccccc5)cc4)c4cccc5oc6ccc(-c7ccccc7)cc6c45)cc3)c2)cc1 ; c1ccc(-c2ccc(N(c3ccc(-c4ccc5c(c4)oc4ccccc45)cc3)c3cccc4oc5ccc(C6CCCCC6)cc5c34)cc2)cc1 ; CC(C)(C)c1ccc2oc3cccc(N(c4ccc(-c5ccccc5)cc4)c4ccc(-c5ccc6oc7ccccc7c6c5)cc4)c3c2c1]

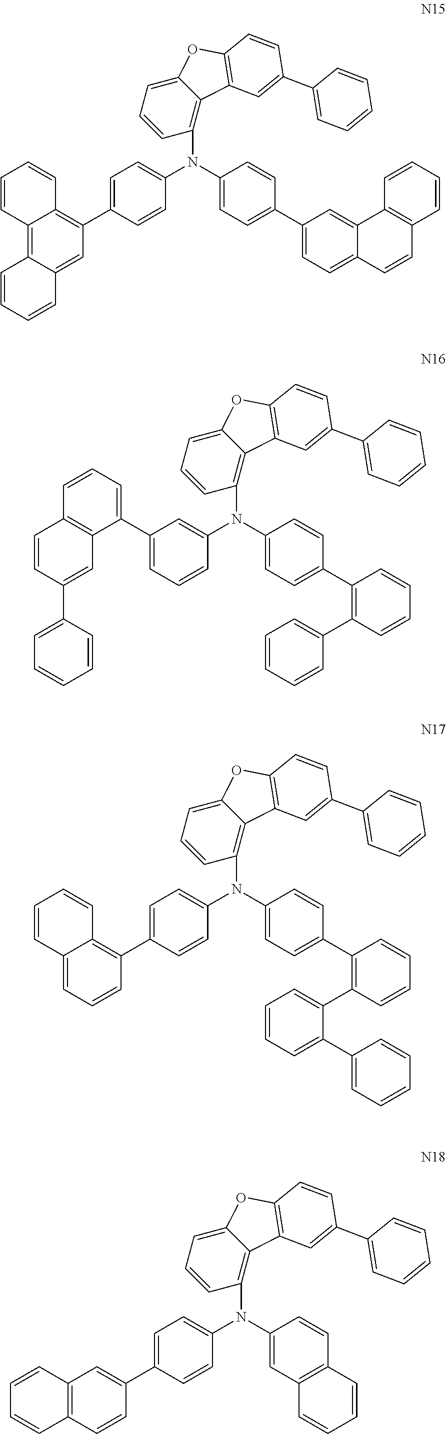 [SMILES: c1ccc(-c2ccc3oc4cccc(N(c5ccc(-c6ccc7ccc8ccccc8c7c6)cc5)c5ccc(-c6cc7ccccc7c7ccccc67)cc5)c4c3c2)cc1 ; c1ccc(-c2ccc3cccc(-c4cccc(N(c5ccc(-c6ccccc6-c6ccccc6)cc5)c5cccc6oc7ccc(-c8ccccc8)cc7c56)c4)c3c2)cc1 ; c1ccc(-c2ccc3oc4cccc(N(c5ccc(-c6ccccc6-c6ccccc6-c6ccccc6)cc5)c5ccc(-c6cccc7ccccc67)cc5)c4c3c2)cc1 ; c1ccc(-c2ccc3oc4cccc(N(c5ccc(-c6ccc7ccccc7c6)cc5)c5ccc6ccccc6c5)c4c3c2)cc1]

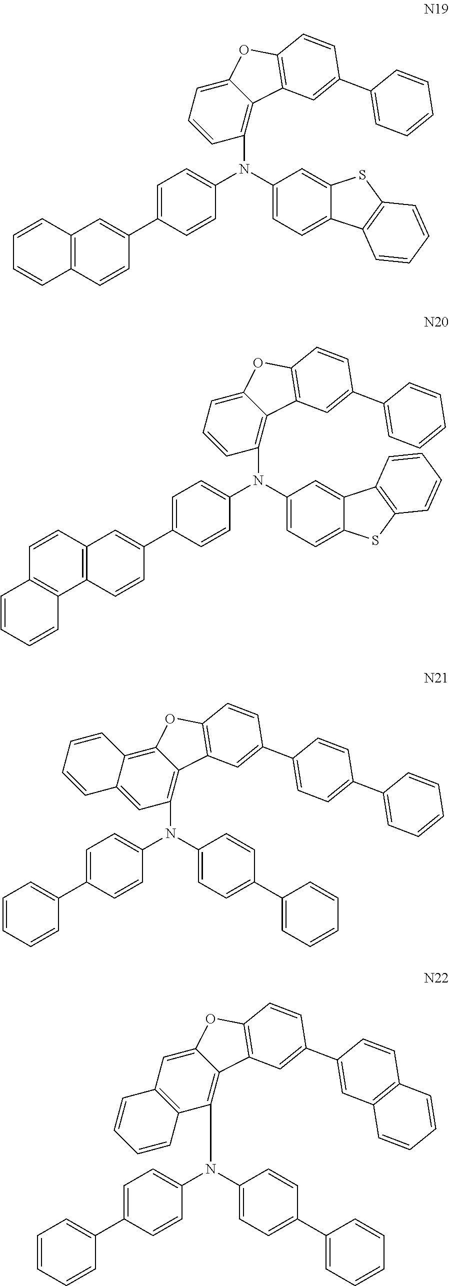 [SMILES: c1ccc(-c2ccc3oc4cccc(N(c5ccc(-c6ccc7ccccc7c6)cc5)c5ccc6c(c5)sc5ccccc56)c4c3c2)cc1 ; c1ccc(-c2ccc3oc4cccc(N(c5ccc(-c6ccc7c(ccc8ccccc87)c6)cc5)c5ccc6sc7ccccc7c6c5)c4c3c2)cc1 ; c1ccc(-c2ccc(-c3ccc4oc5c6ccccc6cc(N(c6ccc(-c7ccccc7)cc6)c6ccc(-c7ccccc7)cc6)c5c4c3)cc2)cc1 ; c1ccc(-c2ccc(N(c3ccc(-c4ccccc4)cc3)c3c4ccccc4cc4oc5ccc(-c6ccc7ccccc7c6)cc5c34)cc2)cc1]

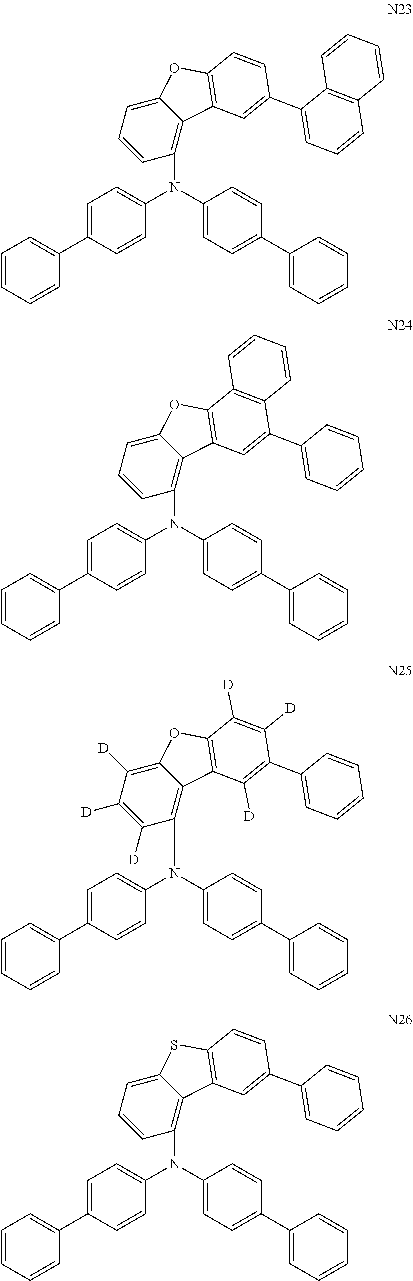 [SMILES: c1ccc(-c2ccc(N(c3ccc(-c4ccccc4)cc3)c3cccc4oc5ccc(-c6cccc7ccccc67)cc5c34)cc2)cc1 ; c1ccc(-c2ccc(N(c3ccc(-c4ccccc4)cc3)c3cccc4oc5c6ccccc6c(-c6ccccc6)cc5c34)cc2)cc1 ; [2H]c1c(-c2ccccc2)c([2H])c2c(oc3c([2H])c([2H])c([2H])c(N(c4ccc(-c5ccccc5)cc4)c4ccc(-c5ccccc5)cc4)c32)c1[2H] ; c1ccc(-c2ccc(N(c3ccc(-c4ccccc4)cc3)c3cccc4sc5ccc(-c6ccccc6)cc5c34)cc2)cc1]

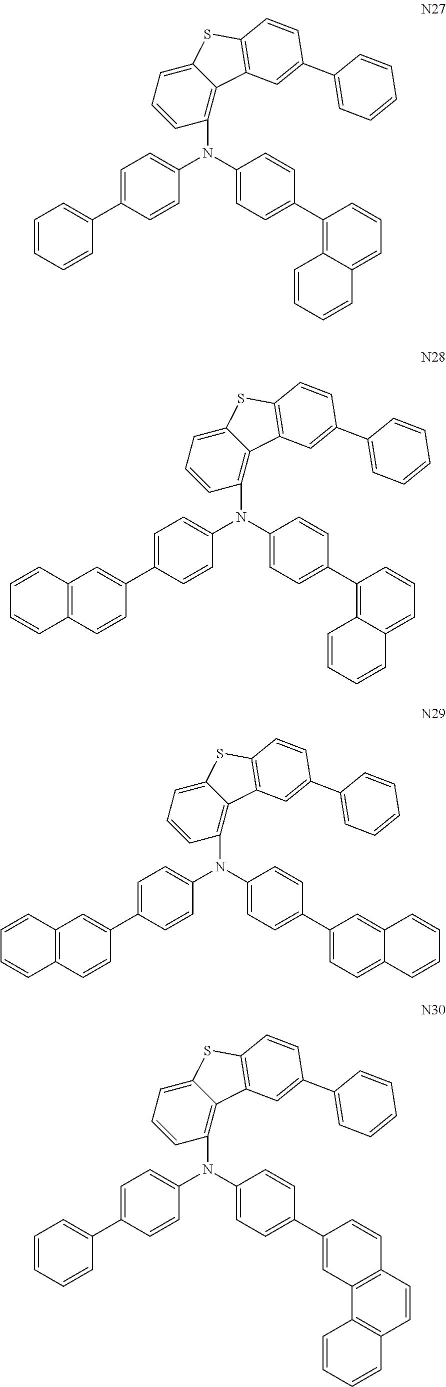 [SMILES: c1ccc(-c2ccc(N(c3ccc(-c4cccc5ccccc45)cc3)c3cccc4sc5ccc(-c6ccccc6)cc5c34)cc2)cc1 ; c1ccc(-c2ccc3sc4cccc(N(c5ccc(-c6ccc7ccccc7c6)cc5)c5ccc(-c6cccc7ccccc67)cc5)c4c3c2)cc1 ; c1ccc(-c2ccc3sc4cccc(N(c5ccc(-c6ccc7ccccc7c6)cc5)c5ccc(-c6ccc7ccccc7c6)cc5)c4c3c2)cc1 ; c1ccc(-c2ccc(N(c3ccc(-c4ccc5ccc6ccccc6c5c4)cc3)c3cccc4sc5ccc(-c6ccccc6)cc5c34)cc2)cc1]

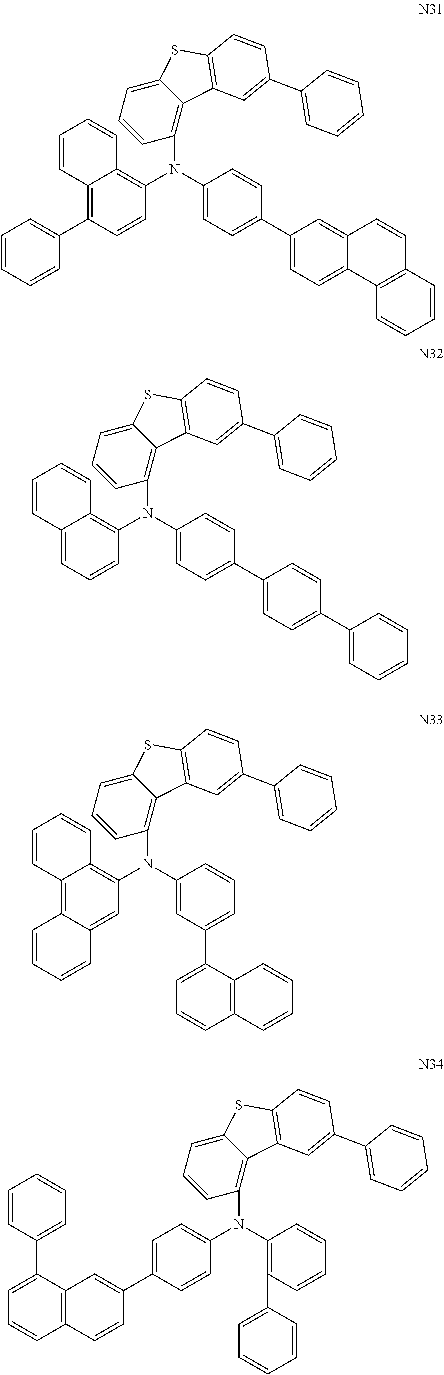 [SMILES: c1ccc(-c2ccc3sc4cccc(N(c5ccc(-c6ccc7c(ccc8ccccc87)c6)cc5)c5ccc(-c6ccccc6)c6ccccc56)c4c3c2)cc1 ; c1ccc(-c2ccc(-c3ccc(N(c4cccc5ccccc45)c4cccc5sc6ccc(-c7ccccc7)cc6c45)cc3)cc2)cc1 ; c1ccc(-c2ccc3sc4cccc(N(c5cccc(-c6cccc7ccccc67)c5)c5cc6ccccc6c6ccccc56)c4c3c2)cc1 ; c1ccc(-c2ccc3sc4cccc(N(c5ccc(-c6ccc7cccc(-c8ccccc8)c7c6)cc5)c5ccccc5-c5ccccc5)c4c3c2)cc1]

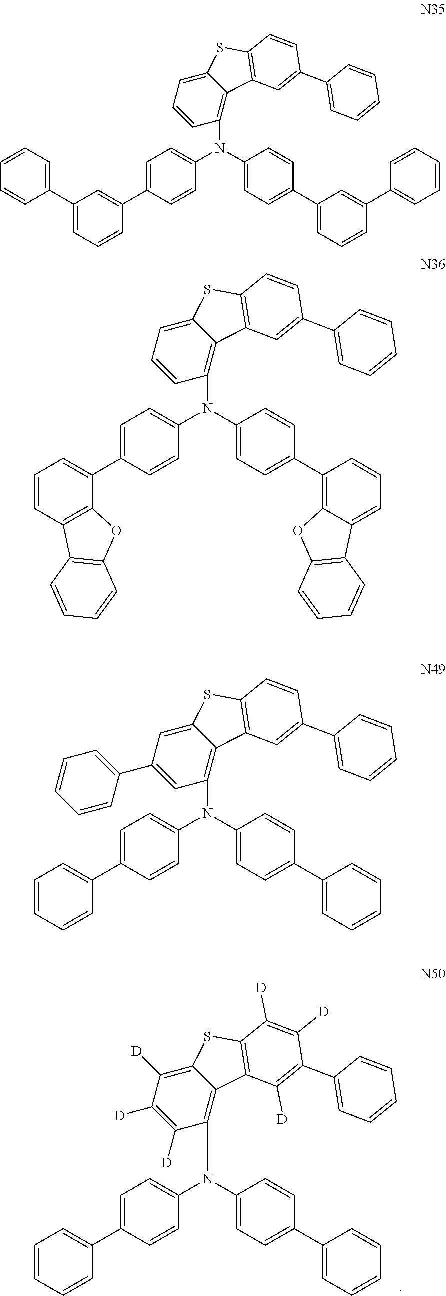 [SMILES: c1ccc(-c2cccc(-c3ccc(N(c4ccc(-c5cccc(-c6ccccc6)c5)cc4)c4cccc5sc6ccc(-c7ccccc7)cc6c45)cc3)c2)cc1 ; c1ccc(-c2ccc3sc4cccc(N(c5ccc(-c6cccc7c6oc6ccccc67)cc5)c5ccc(-c6cccc7c6oc6ccccc67)cc5)c4c3c2)cc1 ; c1ccc(-c2ccc(N(c3ccc(-c4ccccc4)cc3)c3cc(-c4ccccc4)cc4sc5ccc(-c6ccccc6)cc5c34)cc2)cc1 ; [2H]c1c(-c2ccccc2)c([2H])c2c(sc3c([2H])c([2H])c([2H])c(N(c4ccc(-c5ccccc5)cc4)c4ccc(-c5ccccc5)cc4)c32)c1[2H]]

15. An amine compound represented by Formula 1: 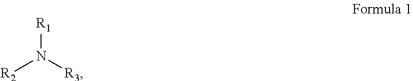
wherein in Formula 1,
R₁ is represented by Formula 2-1, and
R₂ and R₃ are each independently represented by Formula 2-2 or Formula 2-3: 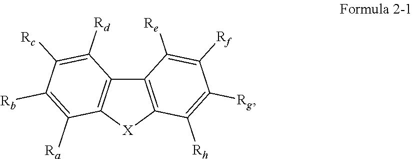
wherein in Formula 2-1,
X is O or S,
Rₐ to R_{d} are each independently a hydrogen atom, a deuterium atom, or a halogen atom, or combined with an adjacent group to form a ring, where any one selected from among Rₐ to R_{d} is a site bonded to nitrogen of Formula 1,
any one selected from among Rₑ to Rₕ is a substituted or unsubstituted alkyl group of 1 to 20 carbon atoms, or a substituted or unsubstituted aryl group of 6 to 30 ring-forming carbon atoms, and the remainder are each independently a hydrogen atom, a deuterium atom, or a halogen atom, or combined with an adjacent group to form a ring, where a fluorenyl group is excluded, and
when R_{d} is bonded to the nitrogen of Formula 1, Rₑ is a hydrogen atom or a deuterium atom, or combined with adjacent R_{f} to form a ring,
*-(L₁)ₘ-Ar₁, Formula 2-2
wherein in Formula 2-2,
L₁ is a substituted or unsubstituted arylene group of 6 to 30 ring-forming carbon atoms,
“m” is an integer of 0 to 3, and
Ar₁ is a substituted or unsubstituted aryl group of 6 to 30 ring-forming carbon atoms, where a fluorenyl group is excluded, and when R₂ and R₃ in Formula 1 are both represented by Formula 2-2, Ar₁ is not a 1-naphthyl group, 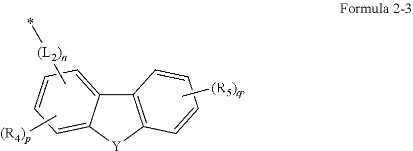
and
wherein in Formula 2-3,
Y is O or S,
L₂ is a substituted or unsubstituted arylene group of 6 to 30 ring-forming carbon atoms,
R₄ is a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, and/or combined with an adjacent group to form a ring,
R₅ is a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring,
“n” and “p” are each independently an integer of 0 to 3, and
“q” is an integer of 0 to 4,
provided that Formula 2-1 and Formula 2-3 are not the same,
when Rₐ of Formula 2-1 is bonded to the nitrogen of Formula 1, and Rₕ is an alkyl group or an aryl group, at least one selected from among R₂ and R₃ of Formula 1 is represented by Formula 2-3,
when X of Formula 2-1 is S, Formula 2-3 is not a 4-dibenzothiophenyl group, and
at least one hydrogen in the amine compound represented by Formula 1 is optionally substituted with deuterium.

16. The amine compound of claim 15, wherein Formula 1 is represented by Formula 3-1 or Formula 3-2: 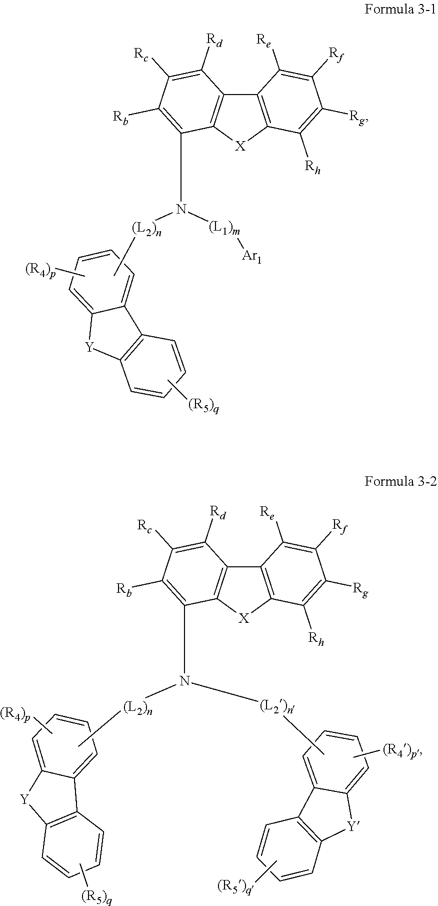
and
wherein in Formula 3-1 and Formula 3-2,
Rₕ is a substituted or unsubstituted alkyl group of 1 to 20 carbon atoms or a substituted or unsubstituted aryl group of 6 to 30 ring-forming carbon atoms,
R_{b} to R_{g} are each independently a hydrogen atom, a deuterium atom, or a halogen atom, or combined with an adjacent group to form a ring,
Y′ is O or S,
L₂′ is a substituted or unsubstituted arylene group of 6 to 30 ring-forming carbon atoms,
R₄′ is a hydrogen atom, a deuterium atom, a halogen atom, or a substituted or unsubstituted alkyl group of 1 to 20 carbon atoms, and/or combined with an adjacent group to form a ring,
R₅′ is a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring,
n′ and p′ are each independently an integer of 0 to 3,
q′ is an integer of 0 to 4, and
X, Ar₁, Y, L₁, L₂, R₄, R₅, “m”, “n”, “p” and “q” are each independently the same as defined in Formula 2-1 to Formula 2-3.

17. The amine compound of claim 15, wherein Formula 1 is represented by any one selected from among Formula 4-1 to Formula 4-3: 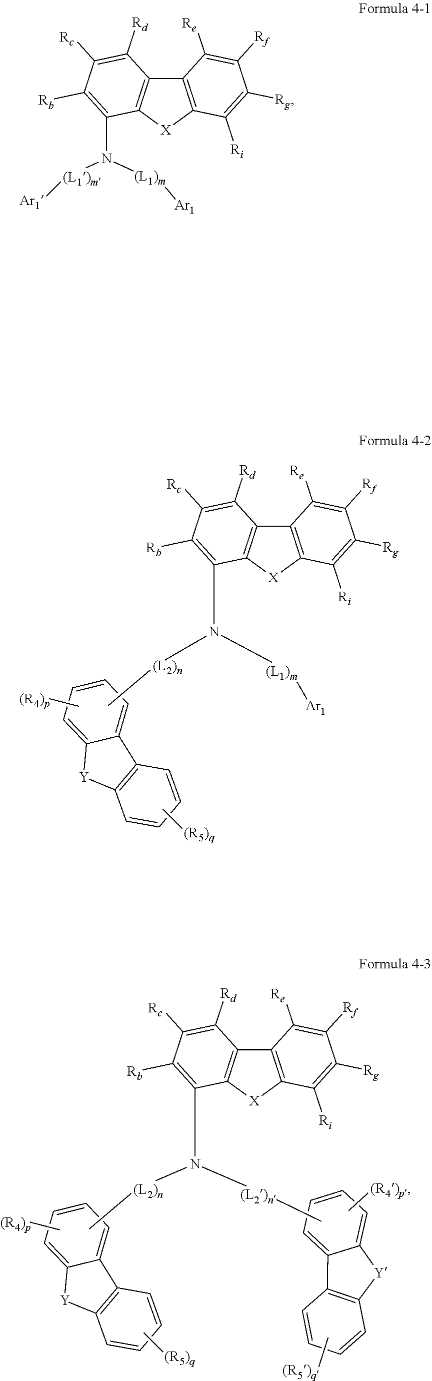
and
wherein in Formula 4-1 to Formula 4-3,
Rᵢ is a substituted or a deuterium atom, or combined with an adjacent R_{g} group to form a ring,
R_{b} to R_{d} are each independently a hydrogen atom, a deuterium atom, or a halogen atom, or combined with an adjacent group to form a ring,
any one selected from among Rₑ to R_{g} is a substituted or unsubstituted alkyl group of 1 to 20 carbon atoms or a substituted or unsubstituted aryl group of 6 to 30 ring-forming carbon atoms, and the remainder are each independently a hydrogen atom, a deuterium atom, or a halogen atom, or combined with an adjacent group to form a ring, where a fluorenyl group is excluded,
L₁′ and L₂′ are each independently a substituted or unsubstituted arylene group of 6 to 30 ring-forming carbon atoms,
R₄′ is a hydrogen atom, a deuterium atom, a halogen atom, or a substituted or unsubstituted alkyl group of 1 to 20 carbon atoms, and/or combined with an adjacent group to form a ring,
R₅′ is a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring,
n′ and p′ are each independently an integer of 0 to 3,
q′ is an integer of 0 to 4, and
Ar₁ and Ar₁′ are each independently a substituted or unsubstituted aryl group of 6 to 30 ring-forming carbon atoms, where a fluorenyl group is excluded,
m′ is an integer of 0 to 3, and
X, Y, L₁, L₂, R₄, R₅, “m”, “n”, “p” and “q” are each independently the same as defined in Formula 2-1 to Formula 2-3, where Ar₁ and Ar₁′ in Formula 4-1 are not simultaneously 1-naphthyl groups.

18. The amine compound of claim 15, wherein Formula 1 is represented by any one selected from among Formula 5-1 to Formula 5-3: 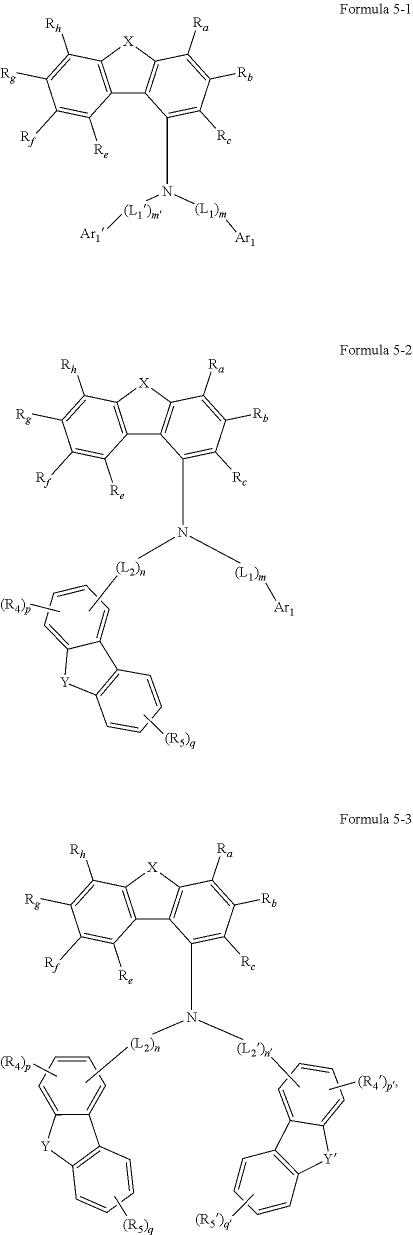
and
wherein in Formula 5-1 to Formula 5-3,
Rₑ is a hydrogen atom or a deuterium atom, or combined with an adjacent R_{f} group to form a ring,
Rₐ to R_{c} are each independently a hydrogen atom, a deuterium atom, or a halogen atom, or combined with an adjacent group to form a ring,
any one selected from among R_{f} to Rₕ is a substituted or unsubstituted alkyl group of 1 to 20 carbon atoms or a substituted or unsubstituted aryl group of 6 to 30 ring-forming carbon atoms, and the remainder are each independently a hydrogen atom, a deuterium atom, or a halogen atom, or combined with an adjacent group to form a ring, where a fluorenyl group is excluded,
L₁′ is a substituted or unsubstituted arylene group of 6 to 30 ring-forming carbon atoms,
Ar₁ and Ar₁′ are each independently a substituted or unsubstituted aryl group of 6 to 30 ring-forming carbon atoms, where a fluorenyl group is excluded,
m′ is an integer of 0 to 3,
Y′ is O or S,
L₂′ is a substituted or unsubstituted arylene group of 6 to 30 ring-forming carbon atoms,
R₄′ is a hydrogen atom, a deuterium atom, a halogen atom, or a substituted or unsubstituted alkyl group of 1 to 20 carbon atoms, and/or combined with an adjacent group to form a ring,
R₅′ is a hydrogen atom, a deuterium atom, a halogen atom, a substituted or unsubstituted alkyl group of 1 to 20 carbon atoms, or a substituted or unsubstituted aryl group of 6 to 30 ring-forming carbon atoms, and/or combined with an adjacent group to form a ring,
n′ and p′ are each independently an integer of 0 to 3,
q′ is an integer of 0 to 4, and
X, Y, L₁, L₂, R₄, R₅, “m”, “n”, “p” and “q” are each independently the same as defined in Formula 2-1 to Formula 2-3, where Ar₁ and Ar₁′ in Formula 5-1 are not simultaneously 1-naphthyl groups.

19. The amine compound of claim 15, wherein the amine compound represented by Formula 1 is any one selected from among compounds represented in Compound Group 1:

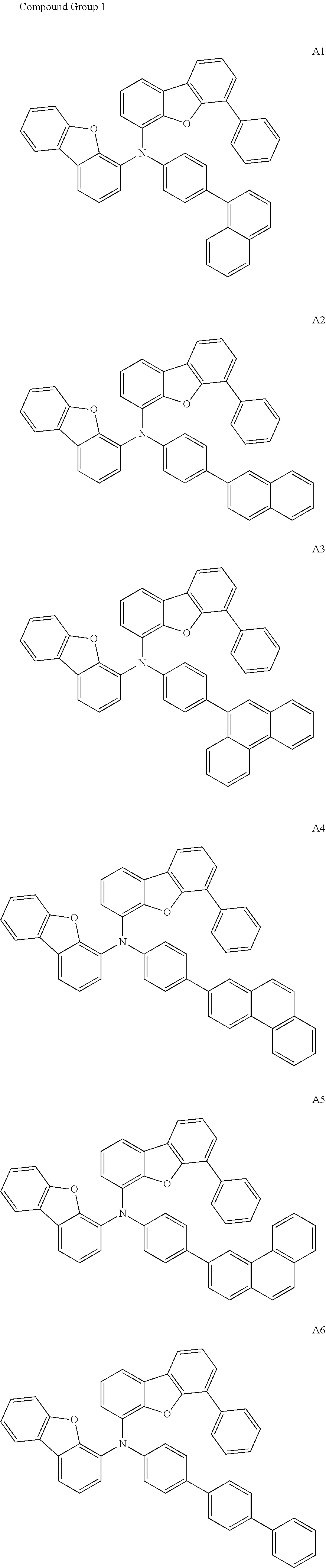

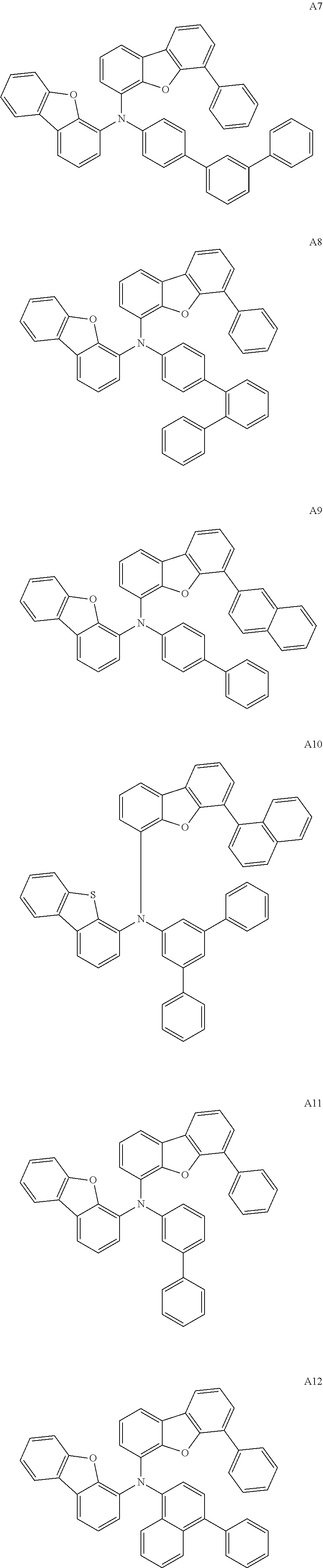

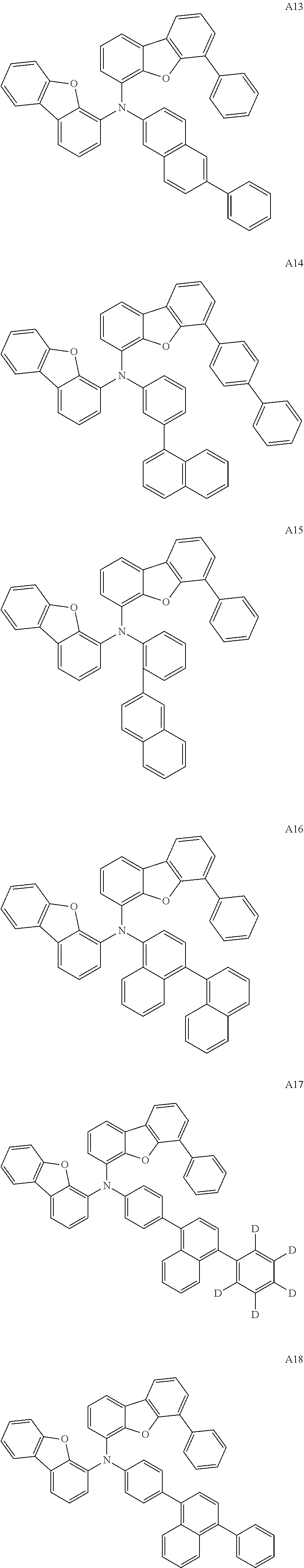

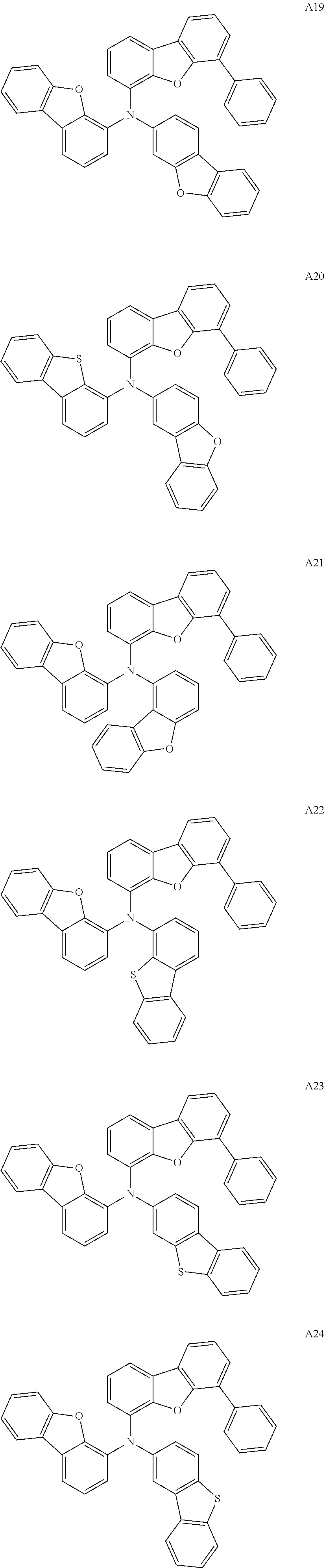 [SMILES: c1ccc(-c2cccc3c2oc2c(N(c4ccc5c(c4)oc4ccccc45)c4cccc5c4oc4ccccc45)cccc23)cc1 ; c1ccc(-c2cccc3c2oc2c(N(c4ccc5oc6ccccc6c5c4)c4cccc5c4sc4ccccc45)cccc23)cc1 ; c1ccc(-c2cccc3c2oc2c(N(c4cccc5c4oc4ccccc45)c4cccc5oc6ccccc6c45)cccc23)cc1 ; c1ccc(-c2cccc3c2oc2c(N(c4cccc5c4oc4ccccc45)c4cccc5c4sc4ccccc45)cccc23)cc1 ; c1ccc(-c2cccc3c2oc2c(N(c4ccc5c(c4)sc4ccccc45)c4cccc5c4oc4ccccc45)cccc23)cc1 ; c1ccc(-c2cccc3c2oc2c(N(c4ccc5sc6ccccc6c5c4)c4cccc5c4oc4ccccc45)cccc23)cc1]

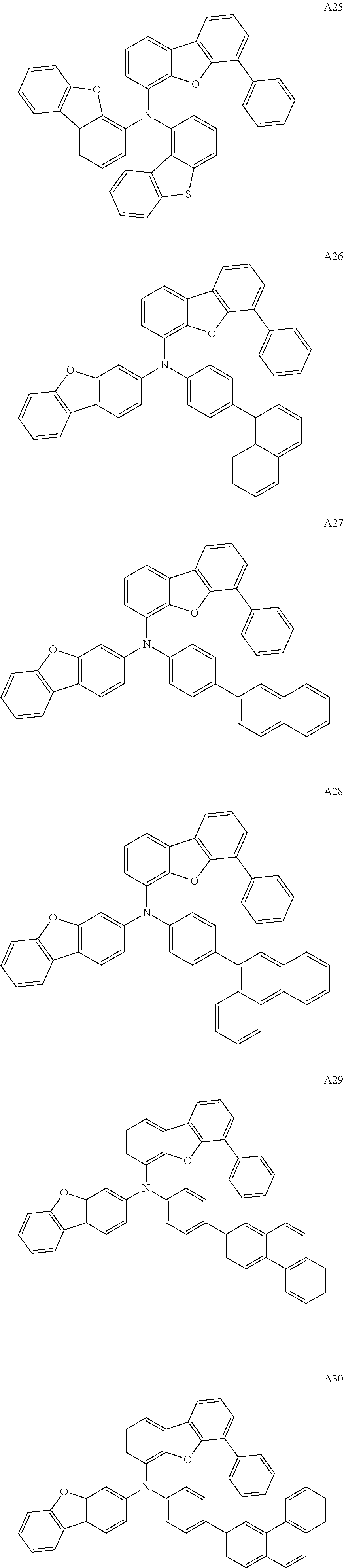

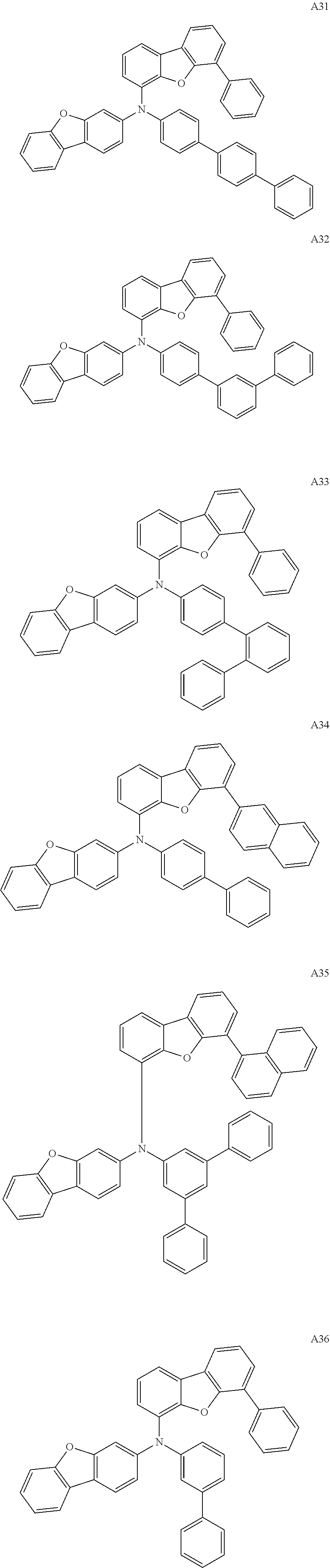

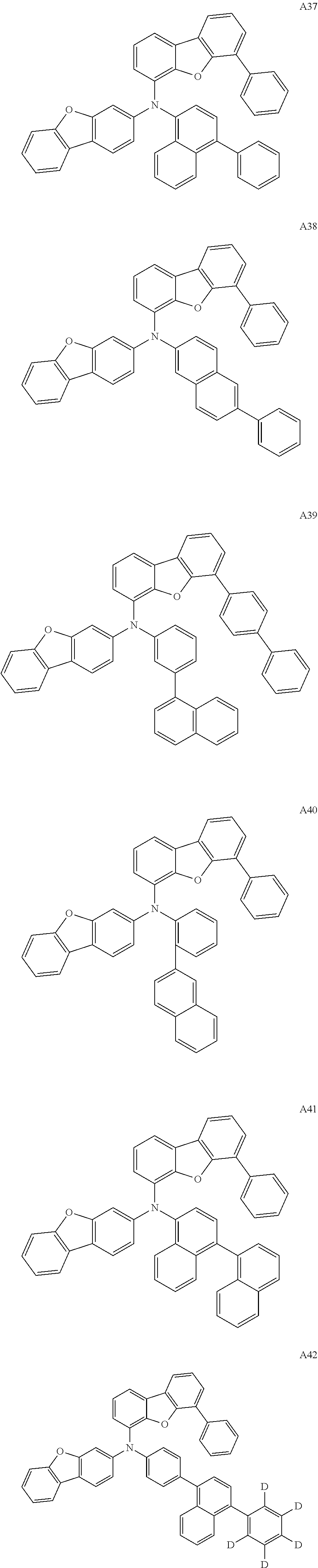

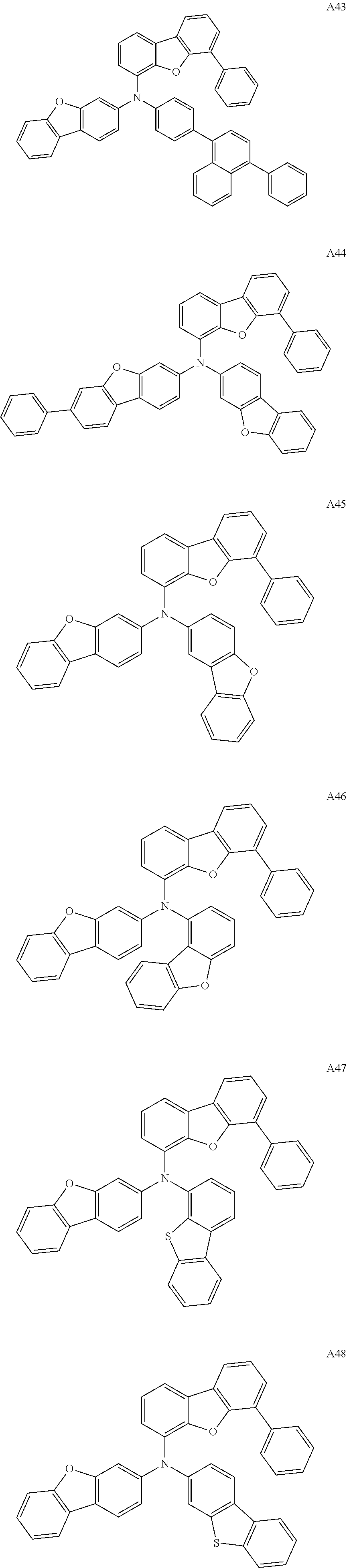 [SMILES: c1ccc(-c2ccc(-c3ccc(N(c4ccc5c(c4)oc4ccccc45)c4cccc5c4oc4c(-c6ccccc6)cccc45)cc3)c3ccccc23)cc1 ; c1ccc(-c2ccc3c(c2)oc2cc(N(c4ccc5c(c4)oc4ccccc45)c4cccc5c4oc4c(-c6ccccc6)cccc45)ccc23)cc1 ; c1ccc(-c2cccc3c2oc2c(N(c4ccc5c(c4)oc4ccccc45)c4ccc5oc6ccccc6c5c4)cccc23)cc1 ; c1ccc(-c2cccc3c2oc2c(N(c4ccc5c(c4)oc4ccccc45)c4cccc5oc6ccccc6c45)cccc23)cc1 ; c1ccc(-c2cccc3c2oc2c(N(c4ccc5c(c4)oc4ccccc45)c4cccc5c4sc4ccccc45)cccc23)cc1 ; c1ccc(-c2cccc3c2oc2c(N(c4ccc5c(c4)oc4ccccc45)c4ccc5c(c4)sc4ccccc45)cccc23)cc1]

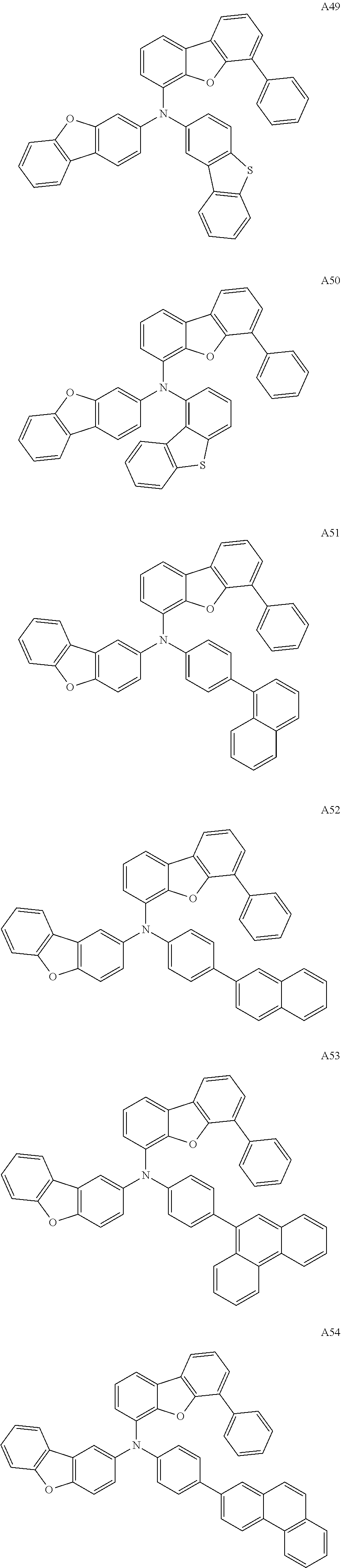 [SMILES: c1ccc(-c2cccc3c2oc2c(N(c4ccc5c(c4)oc4ccccc45)c4ccc5sc6ccccc6c5c4)cccc23)cc1 ; c1ccc(-c2cccc3c2oc2c(N(c4ccc5c(c4)oc4ccccc45)c4cccc5sc6ccccc6c45)cccc23)cc1 ; c1ccc(-c2cccc3c2oc2c(N(c4ccc(-c5cccc6ccccc56)cc4)c4ccc5oc6ccccc6c5c4)cccc23)cc1 ; c1ccc(-c2cccc3c2oc2c(N(c4ccc(-c5ccc6ccccc6c5)cc4)c4ccc5oc6ccccc6c5c4)cccc23)cc1 ; c1ccc(-c2cccc3c2oc2c(N(c4ccc(-c5cc6ccccc6c6ccccc56)cc4)c4ccc5oc6ccccc6c5c4)cccc23)cc1 ; c1ccc(-c2cccc3c2oc2c(N(c4ccc(-c5ccc6c(ccc7ccccc76)c5)cc4)c4ccc5oc6ccccc6c5c4)cccc23)cc1]

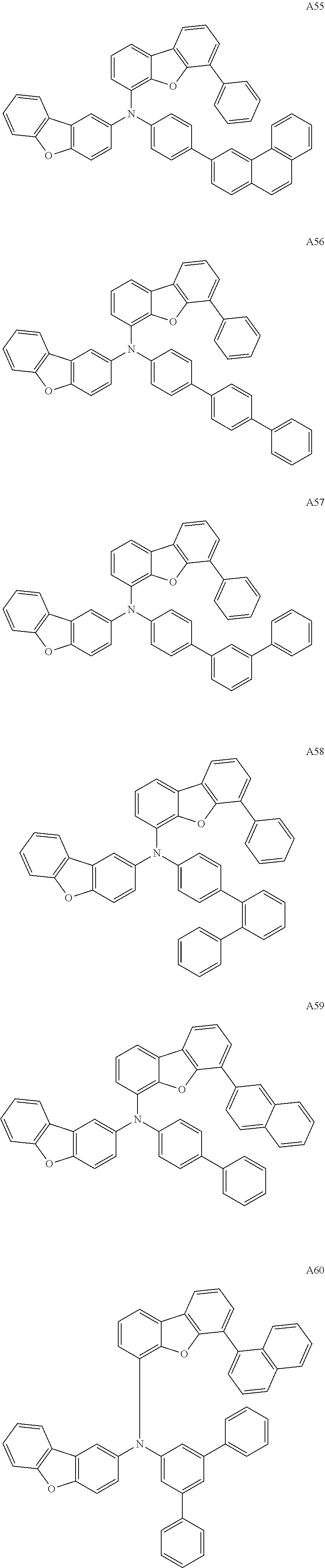

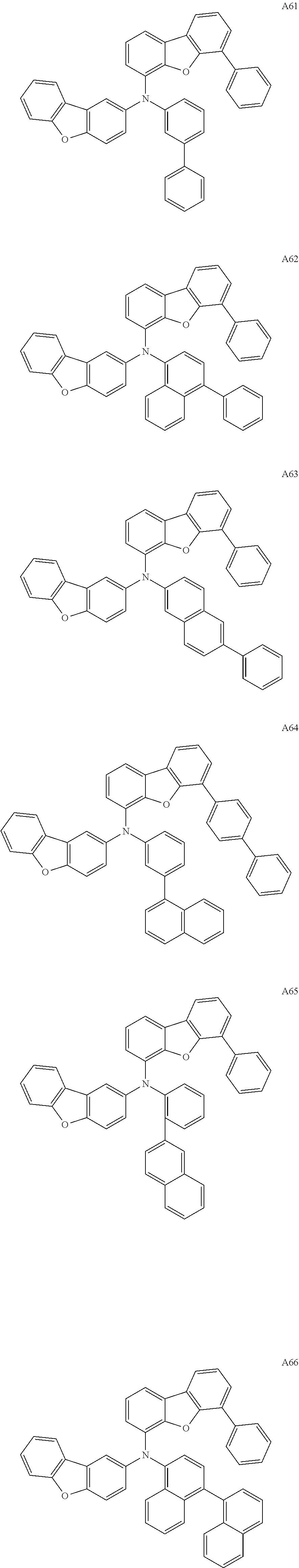 [SMILES: c1ccc(-c2cccc(N(c3ccc4oc5ccccc5c4c3)c3cccc4c3oc3c(-c5ccccc5)cccc34)c2)cc1 ; c1ccc(-c2ccc(N(c3ccc4oc5ccccc5c4c3)c3cccc4c3oc3c(-c5ccccc5)cccc34)c3ccccc23)cc1 ; c1ccc(-c2ccc3cc(N(c4ccc5oc6ccccc6c5c4)c4cccc5c4oc4c(-c6ccccc6)cccc45)ccc3c2)cc1 ; c1ccc(-c2ccc(-c3cccc4c3oc3c(N(c5cccc(-c6cccc7ccccc67)c5)c5ccc6oc7ccccc7c6c5)cccc34)cc2)cc1 ; c1ccc(-c2cccc3c2oc2c(N(c4ccc5oc6ccccc6c5c4)c4ccccc4-c4ccc5ccccc5c4)cccc23)cc1 ; c1ccc(-c2cccc3c2oc2c(N(c4ccc5oc6ccccc6c5c4)c4ccc(-c5cccc6ccccc56)c5ccccc45)cccc23)cc1]

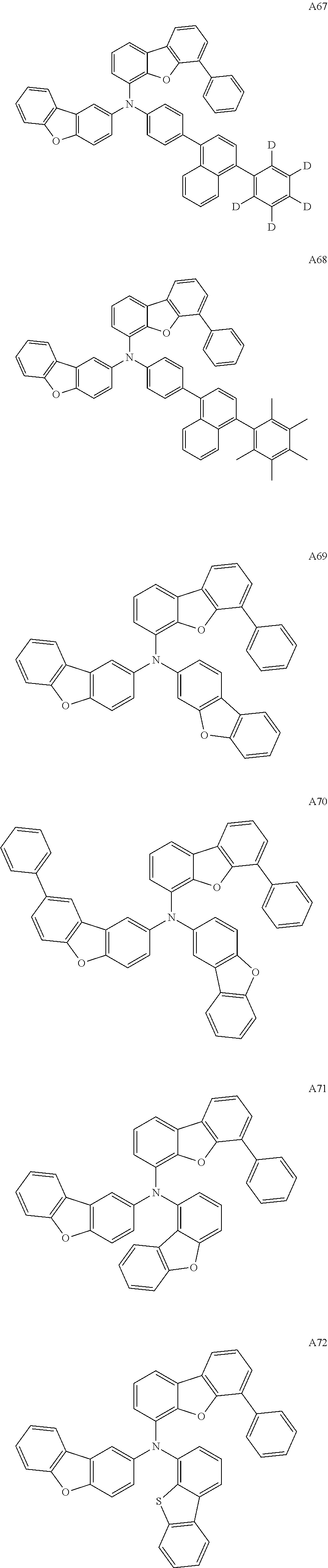

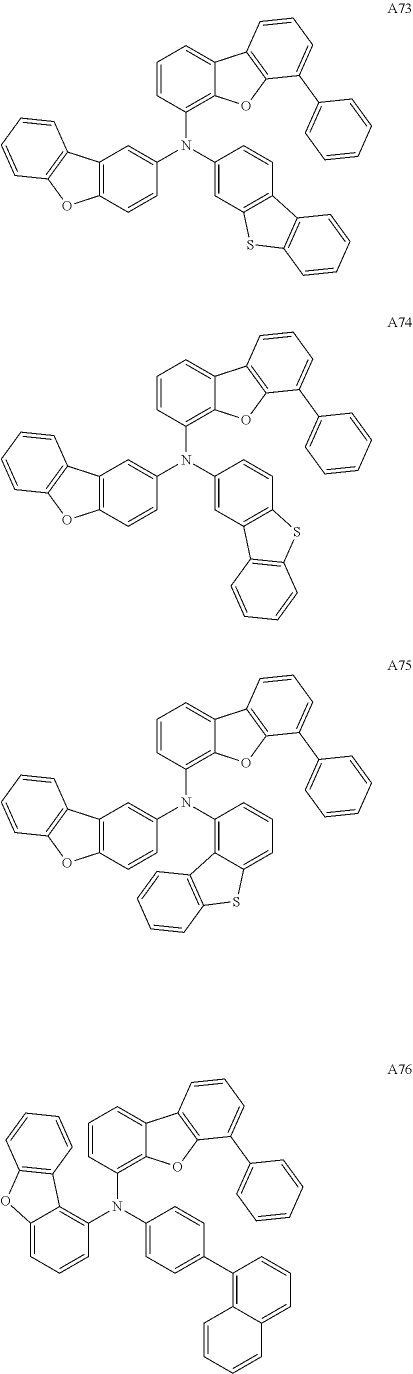 [SMILES: c1ccc(-c2cccc3c2oc2c(N(c4ccc5c(c4)sc4ccccc45)c4ccc5oc6ccccc6c5c4)cccc23)cc1 ; c1ccc(-c2cccc3c2oc2c(N(c4ccc5oc6ccccc6c5c4)c4ccc5sc6ccccc6c5c4)cccc23)cc1 ; c1ccc(-c2cccc3c2oc2c(N(c4ccc5oc6ccccc6c5c4)c4cccc5sc6ccccc6c45)cccc23)cc1 ; c1ccc(-c2cccc3c2oc2c(N(c4ccc(-c5cccc6ccccc56)cc4)c4cccc5oc6ccccc6c45)cccc23)cc1]

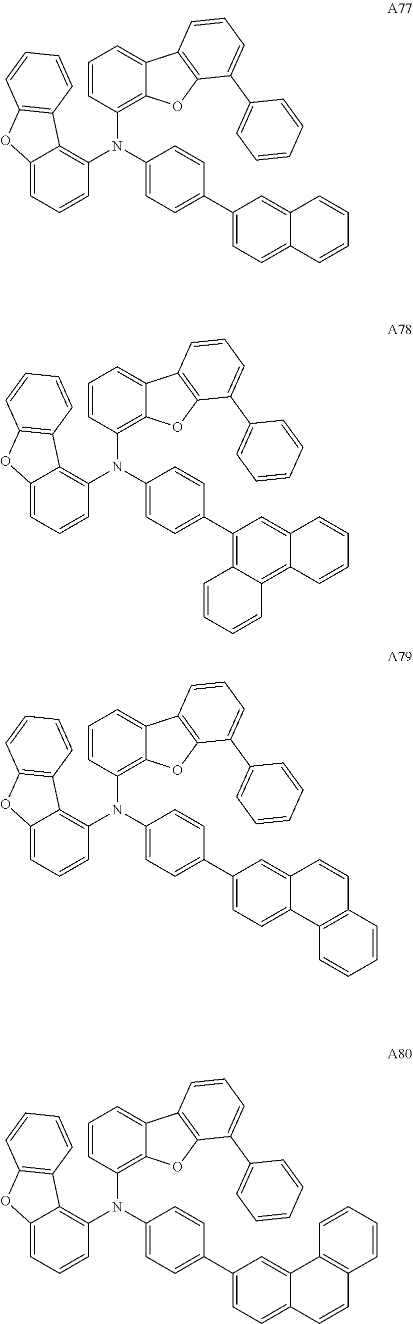 [SMILES: c1ccc(-c2cccc3c2oc2c(N(c4ccc(-c5ccc6ccccc6c5)cc4)c4cccc5oc6ccccc6c45)cccc23)cc1 ; c1ccc(-c2cccc3c2oc2c(N(c4ccc(-c5cc6ccccc6c6ccccc56)cc4)c4cccc5oc6ccccc6c45)cccc23)cc1 ; c1ccc(-c2cccc3c2oc2c(N(c4ccc(-c5ccc6c(ccc7ccccc76)c5)cc4)c4cccc5oc6ccccc6c45)cccc23)cc1 ; c1ccc(-c2cccc3c2oc2c(N(c4ccc(-c5ccc6ccc7ccccc7c6c5)cc4)c4cccc5oc6ccccc6c45)cccc23)cc1]

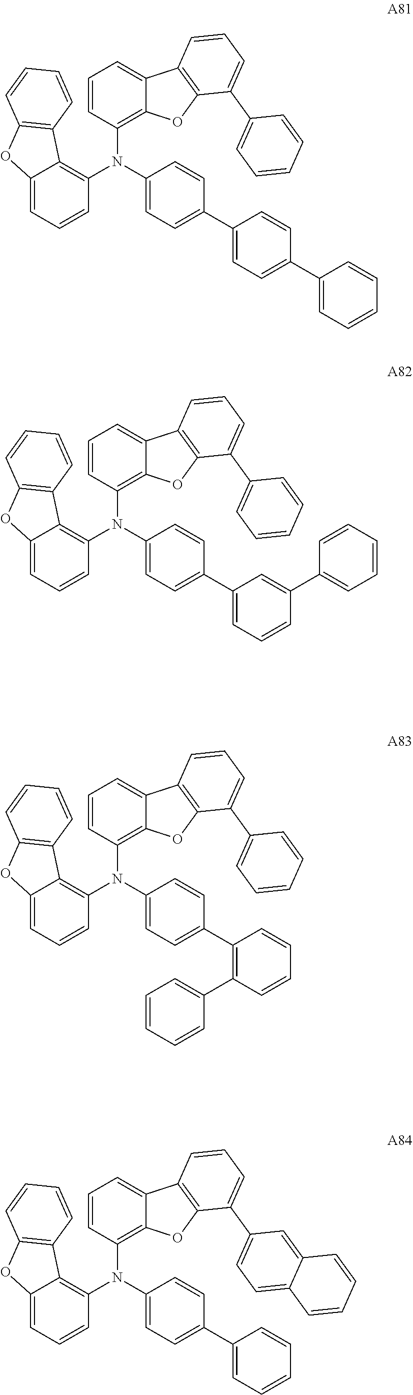 [SMILES: c1ccc(-c2ccc(-c3ccc(N(c4cccc5c4oc4c(-c6ccccc6)cccc45)c4cccc5oc6ccccc6c45)cc3)cc2)cc1 ; c1ccc(-c2cccc(-c3ccc(N(c4cccc5c4oc4c(-c6ccccc6)cccc45)c4cccc5oc6ccccc6c45)cc3)c2)cc1 ; c1ccc(-c2ccccc2-c2ccc(N(c3cccc4c3oc3c(-c5ccccc5)cccc34)c3cccc4oc5ccccc5c34)cc2)cc1 ; c1ccc(-c2ccc(N(c3cccc4c3oc3c(-c5ccc6ccccc6c5)cccc34)c3cccc4oc5ccccc5c34)cc2)cc1]

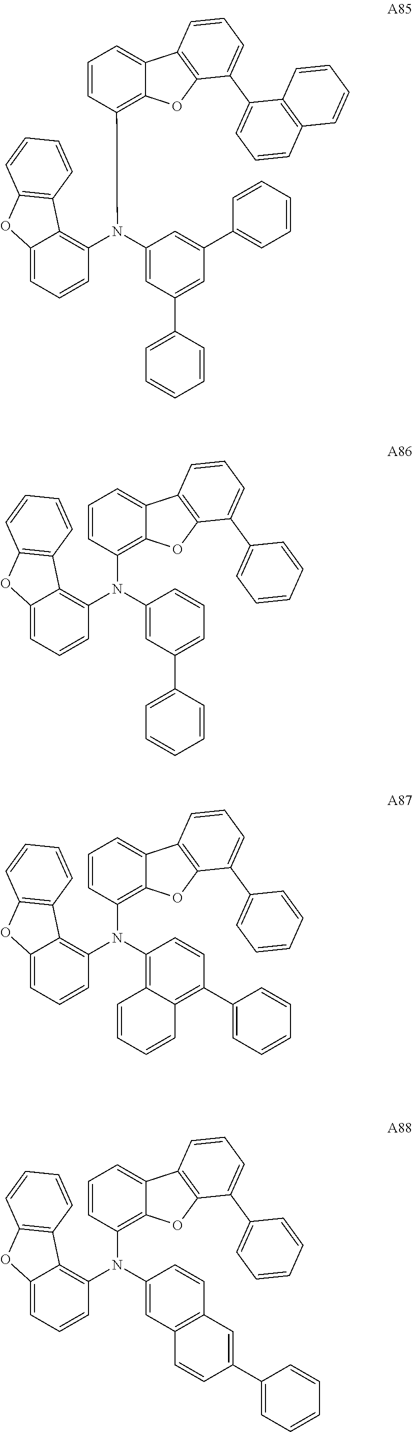 [SMILES: c1ccc(-c2cc(-c3ccccc3)cc(N(c3cccc4c3oc3c(-c5cccc6ccccc56)cccc34)c3cccc4oc5ccccc5c34)c2)cc1 ; c1ccc(-c2cccc(N(c3cccc4c3oc3c(-c5ccccc5)cccc34)c3cccc4oc5ccccc5c34)c2)cc1 ; c1ccc(-c2ccc(N(c3cccc4c3oc3c(-c5ccccc5)cccc34)c3cccc4oc5ccccc5c34)c3ccccc23)cc1 ; c1ccc(-c2ccc3cc(N(c4cccc5c4oc4c(-c6ccccc6)cccc45)c4cccc5oc6ccccc6c45)ccc3c2)cc1]

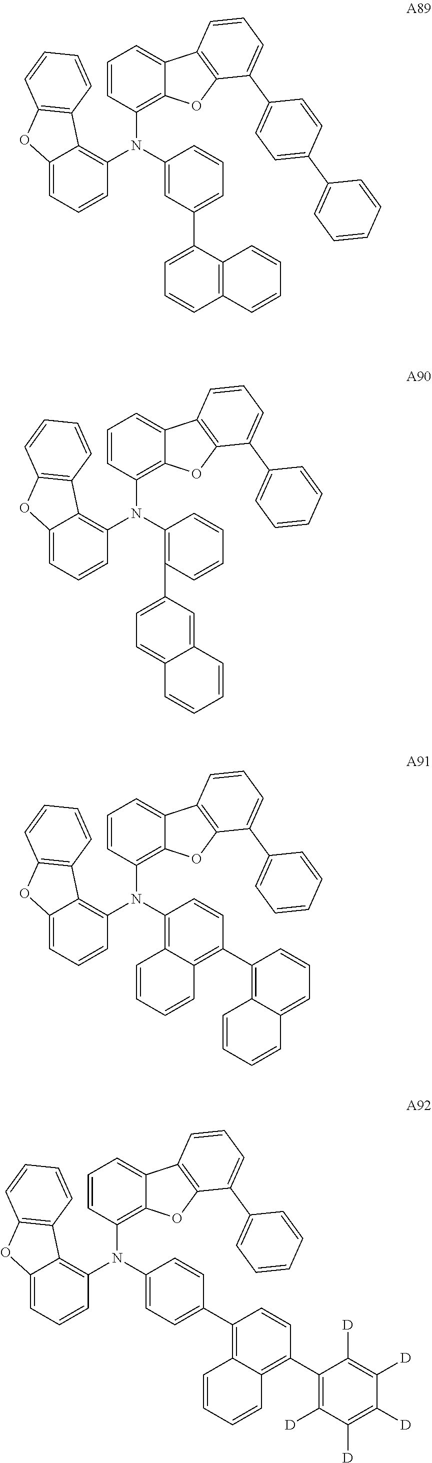 [SMILES: c1ccc(-c2ccc(-c3cccc4c3oc3c(N(c5cccc(-c6cccc7ccccc67)c5)c5cccc6oc7ccccc7c56)cccc34)cc2)cc1 ; c1ccc(-c2cccc3c2oc2c(N(c4ccccc4-c4ccc5ccccc5c4)c4cccc5oc6ccccc6c45)cccc23)cc1 ; c1ccc(-c2cccc3c2oc2c(N(c4ccc(-c5cccc6ccccc56)c5ccccc45)c4cccc5oc6ccccc6c45)cccc23)cc1 ; [2H]c1c([2H])c([2H])c(-c2ccc(-c3ccc(N(c4cccc5c4oc4c(-c6ccccc6)cccc45)c4cccc5oc6ccccc6c45)cc3)c3ccccc23)c([2H])c1[2H]]

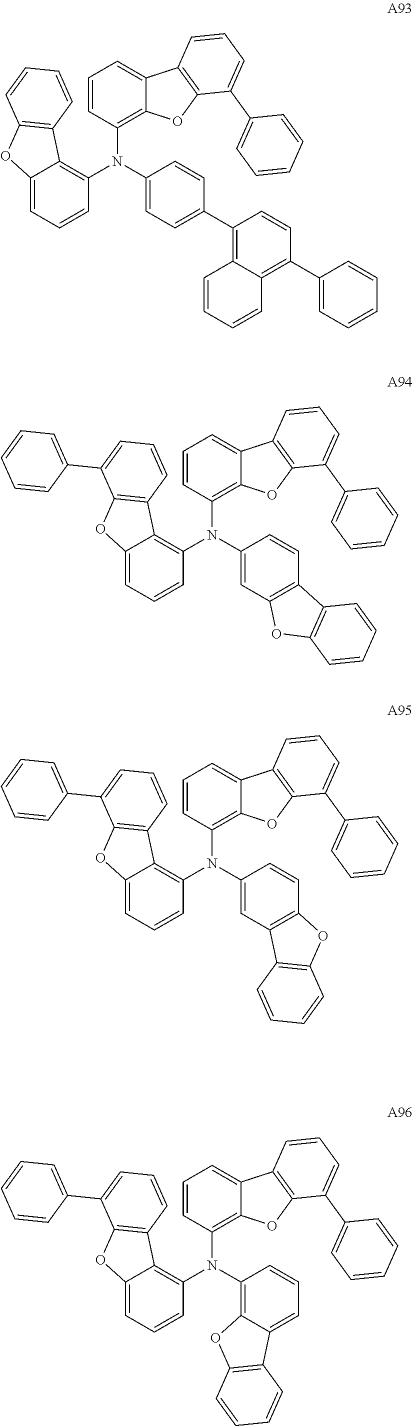 [SMILES: c1ccc(-c2ccc(-c3ccc(N(c4cccc5c4oc4c(-c6ccccc6)cccc45)c4cccc5oc6ccccc6c45)cc3)c3ccccc23)cc1 ; c1ccc(-c2cccc3c2oc2c(N(c4ccc5c(c4)oc4ccccc45)c4cccc5oc6c(-c7ccccc7)cccc6c45)cccc23)cc1 ; c1ccc(-c2cccc3c2oc2c(N(c4ccc5oc6ccccc6c5c4)c4cccc5oc6c(-c7ccccc7)cccc6c45)cccc23)cc1 ; c1ccc(-c2cccc3c2oc2c(N(c4cccc5c4oc4ccccc45)c4cccc5oc6c(-c7ccccc7)cccc6c45)cccc23)cc1]

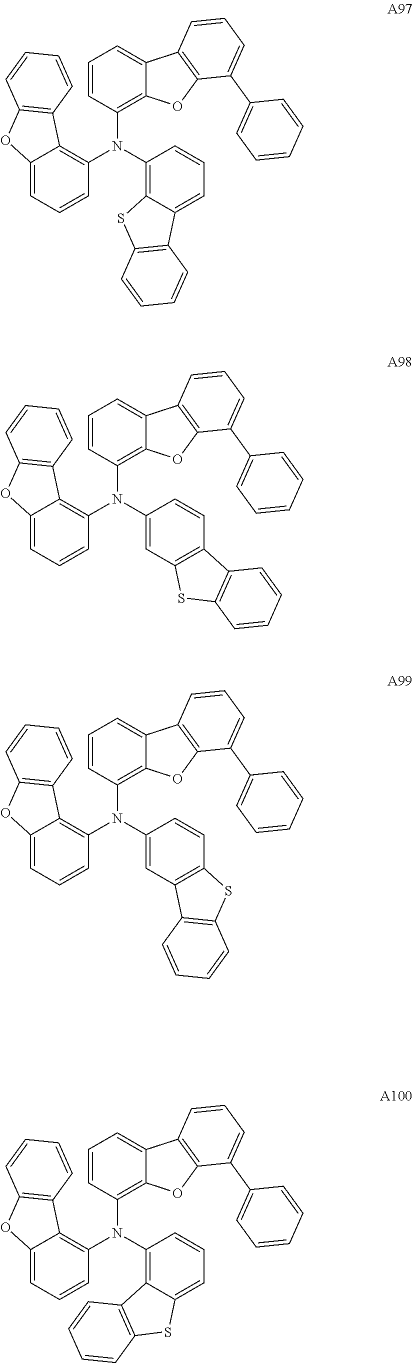 [SMILES: c1ccc(-c2cccc3c2oc2c(N(c4cccc5c4sc4ccccc45)c4cccc5oc6ccccc6c45)cccc23)cc1 ; c1ccc(-c2cccc3c2oc2c(N(c4ccc5c(c4)sc4ccccc45)c4cccc5oc6ccccc6c45)cccc23)cc1 ; c1ccc(-c2cccc3c2oc2c(N(c4ccc5sc6ccccc6c5c4)c4cccc5oc6ccccc6c45)cccc23)cc1 ; c1ccc(-c2cccc3c2oc2c(N(c4cccc5oc6ccccc6c45)c4cccc5sc6ccccc6c45)cccc23)cc1]

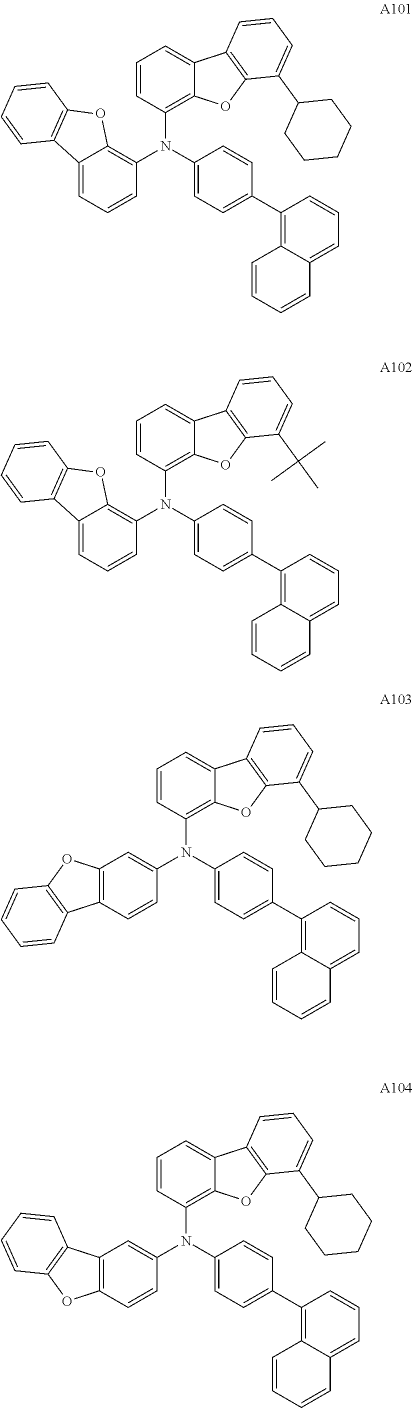 [SMILES: c1ccc2c(-c3ccc(N(c4cccc5c4oc4ccccc45)c4cccc5c4oc4c(C6CCCCC6)cccc45)cc3)cccc2c1 ; CC(C)(C)c1cccc2c1oc1c(N(c3ccc(-c4cccc5ccccc45)cc3)c3cccc4c3oc3ccccc34)cccc12 ; c1ccc2c(-c3ccc(N(c4ccc5c(c4)oc4ccccc45)c4cccc5c4oc4c(C6CCCCC6)cccc45)cc3)cccc2c1 ; c1ccc2c(-c3ccc(N(c4ccc5oc6ccccc6c5c4)c4cccc5c4oc4c(C6CCCCC6)cccc45)cc3)cccc2c1]

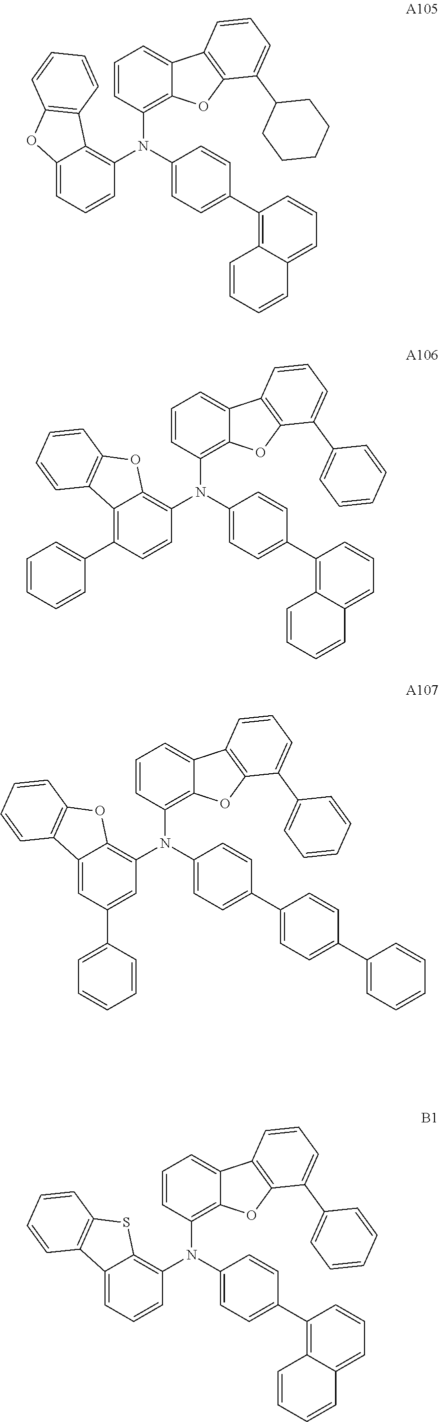 [SMILES: c1ccc2c(-c3ccc(N(c4cccc5c4oc4c(C6CCCCC6)cccc45)c4cccc5oc6ccccc6c45)cc3)cccc2c1 ; c1ccc(-c2cccc3c2oc2c(N(c4ccc(-c5cccc6ccccc56)cc4)c4ccc(-c5ccccc5)c5c4oc4ccccc45)cccc23)cc1 ; c1ccc(-c2ccc(-c3ccc(N(c4cc(-c5ccccc5)cc5c4oc4ccccc45)c4cccc5c4oc4c(-c6ccccc6)cccc45)cc3)cc2)cc1 ; c1ccc(-c2cccc3c2oc2c(N(c4ccc(-c5cccc6ccccc56)cc4)c4cccc5c4sc4ccccc45)cccc23)cc1]

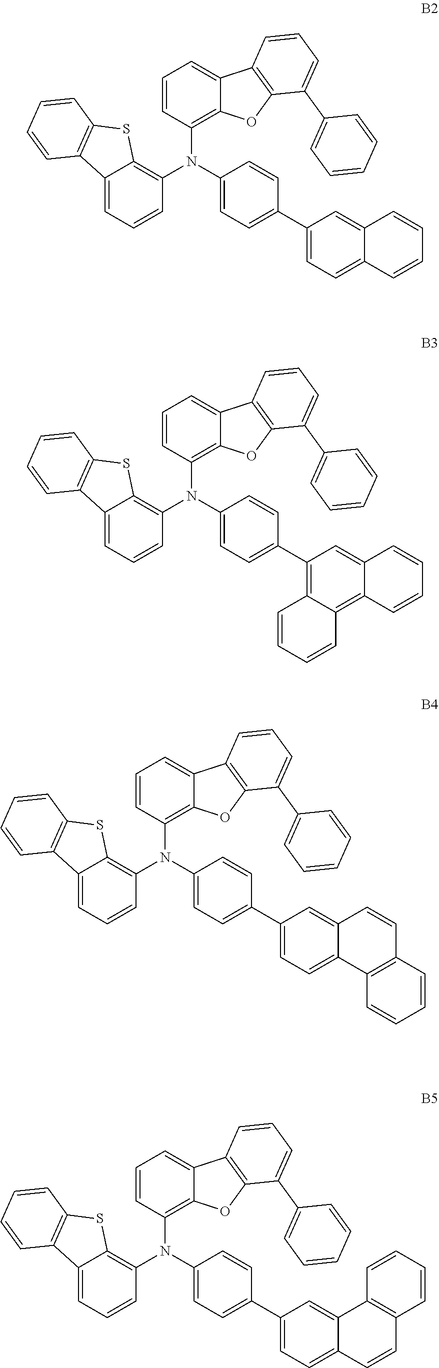 [SMILES: c1ccc(-c2cccc3c2oc2c(N(c4ccc(-c5ccc6ccccc6c5)cc4)c4cccc5c4sc4ccccc45)cccc23)cc1 ; c1ccc(-c2cccc3c2oc2c(N(c4ccc(-c5cc6ccccc6c6ccccc56)cc4)c4cccc5c4sc4ccccc45)cccc23)cc1 ; c1ccc(-c2cccc3c2oc2c(N(c4ccc(-c5ccc6c(ccc7ccccc76)c5)cc4)c4cccc5c4sc4ccccc45)cccc23)cc1 ; c1ccc(-c2cccc3c2oc2c(N(c4ccc(-c5ccc6ccc7ccccc7c6c5)cc4)c4cccc5c4sc4ccccc45)cccc23)cc1]

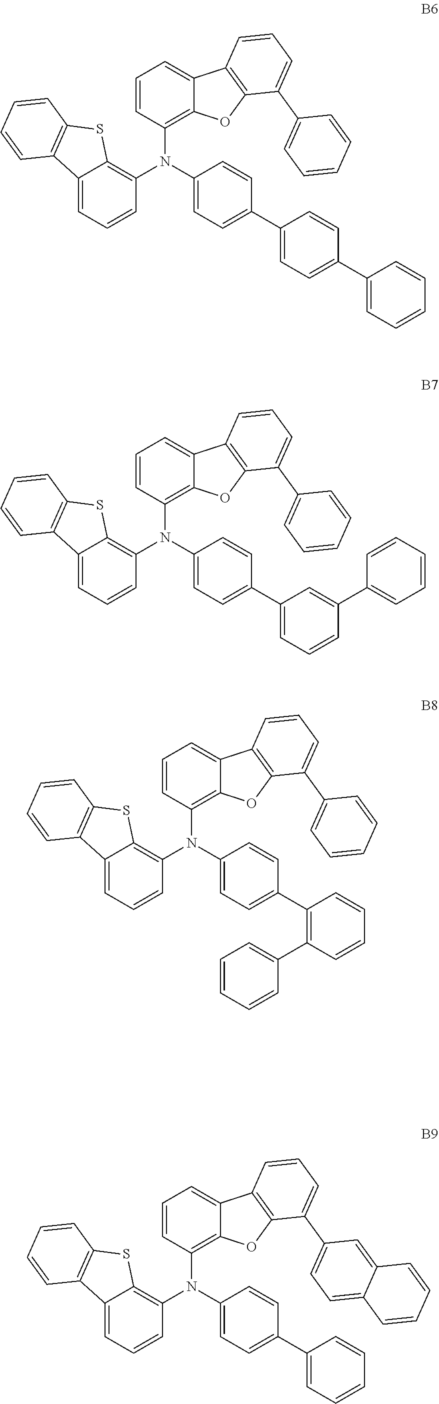 [SMILES: c1ccc(-c2ccc(-c3ccc(N(c4cccc5c4oc4c(-c6ccccc6)cccc45)c4cccc5c4sc4ccccc45)cc3)cc2)cc1 ; c1ccc(-c2cccc(-c3ccc(N(c4cccc5c4oc4c(-c6ccccc6)cccc45)c4cccc5c4sc4ccccc45)cc3)c2)cc1 ; c1ccc(-c2ccccc2-c2ccc(N(c3cccc4c3oc3c(-c5ccccc5)cccc34)c3cccc4c3sc3ccccc34)cc2)cc1 ; c1ccc(-c2ccc(N(c3cccc4c3oc3c(-c5ccc6ccccc6c5)cccc34)c3cccc4c3sc3ccccc34)cc2)cc1]

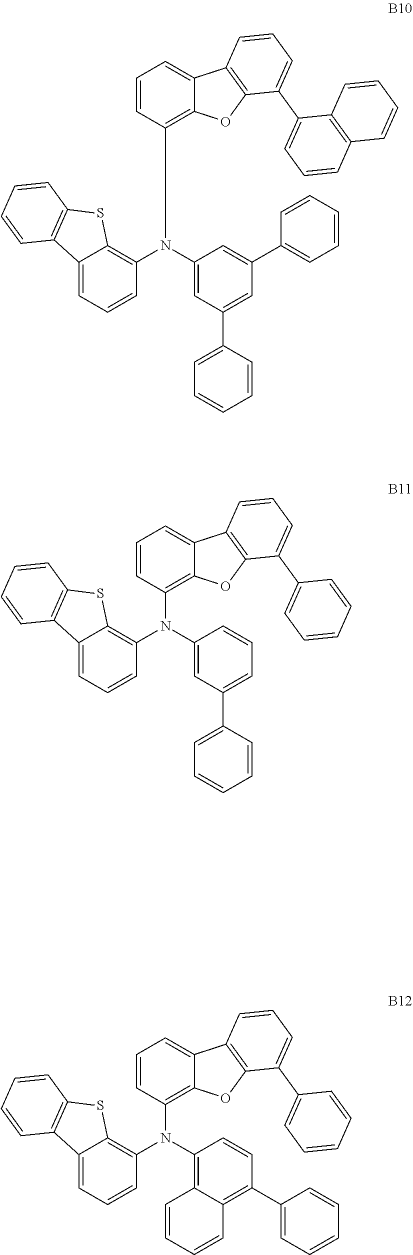 [SMILES: c1ccc(-c2cc(-c3ccccc3)cc(N(c3cccc4c3oc3c(-c5cccc6ccccc56)cccc34)c3cccc4c3sc3ccccc34)c2)cc1 ; c1ccc(-c2cccc(N(c3cccc4c3oc3c(-c5ccccc5)cccc34)c3cccc4c3sc3ccccc34)c2)cc1 ; c1ccc(-c2ccc(N(c3cccc4c3oc3c(-c5ccccc5)cccc34)c3cccc4c3sc3ccccc34)c3ccccc23)cc1]

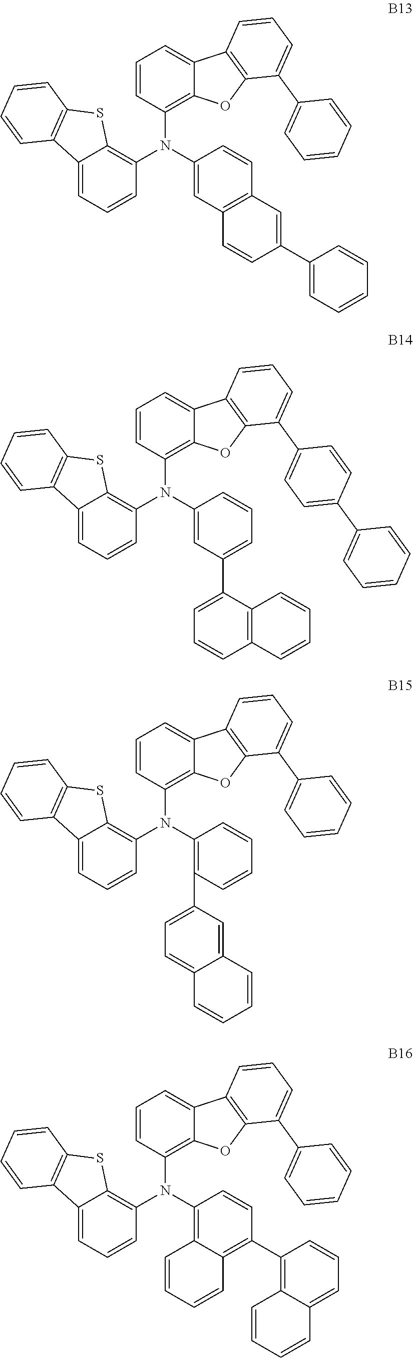

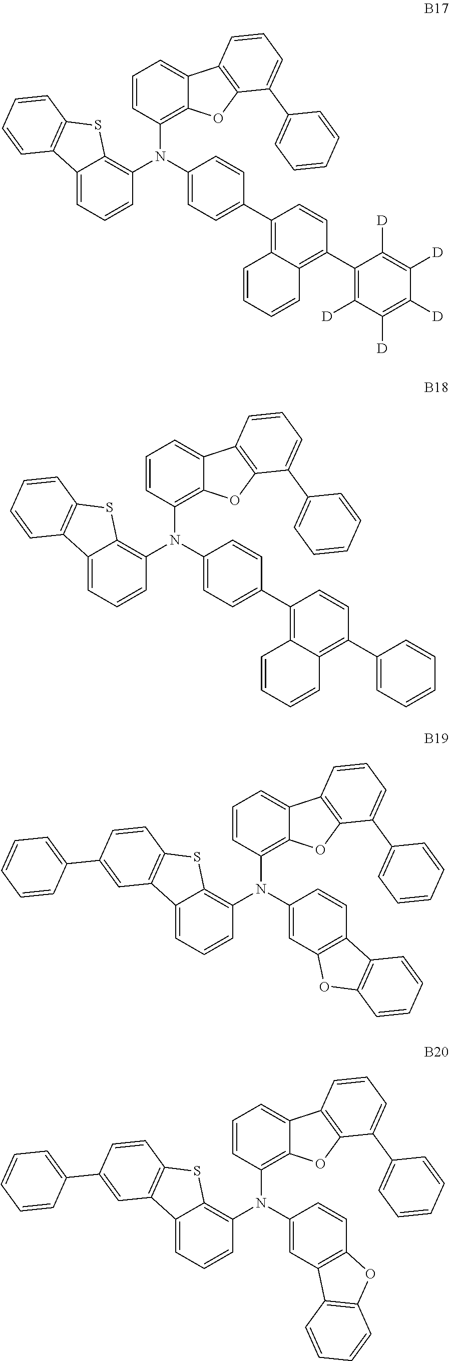 [SMILES: [2H]c1c([2H])c([2H])c(-c2ccc(-c3ccc(N(c4cccc5c4oc4c(-c6ccccc6)cccc45)c4cccc5c4sc4ccccc45)cc3)c3ccccc23)c([2H])c1[2H] ; c1ccc(-c2ccc(-c3ccc(N(c4cccc5c4oc4c(-c6ccccc6)cccc45)c4cccc5c4sc4ccccc45)cc3)c3ccccc23)cc1 ; c1ccc(-c2ccc3sc4c(N(c5ccc6c(c5)oc5ccccc56)c5cccc6c5oc5c(-c7ccccc7)cccc56)cccc4c3c2)cc1 ; c1ccc(-c2ccc3sc4c(N(c5ccc6oc7ccccc7c6c5)c5cccc6c5oc5c(-c7ccccc7)cccc56)cccc4c3c2)cc1]

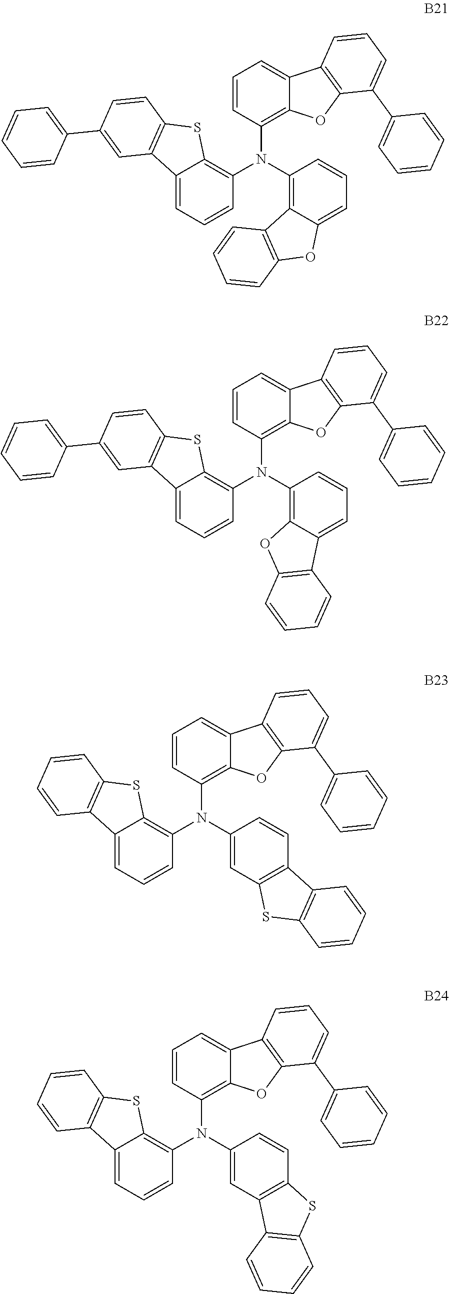 [SMILES: c1ccc(-c2ccc3sc4c(N(c5cccc6c5oc5c(-c7ccccc7)cccc56)c5cccc6oc7ccccc7c56)cccc4c3c2)cc1 ; c1ccc(-c2ccc3sc4c(N(c5cccc6c5oc5ccccc56)c5cccc6c5oc5c(-c7ccccc7)cccc56)cccc4c3c2)cc1 ; c1ccc(-c2cccc3c2oc2c(N(c4ccc5c(c4)sc4ccccc45)c4cccc5c4sc4ccccc45)cccc23)cc1 ; c1ccc(-c2cccc3c2oc2c(N(c4ccc5sc6ccccc6c5c4)c4cccc5c4sc4ccccc45)cccc23)cc1]

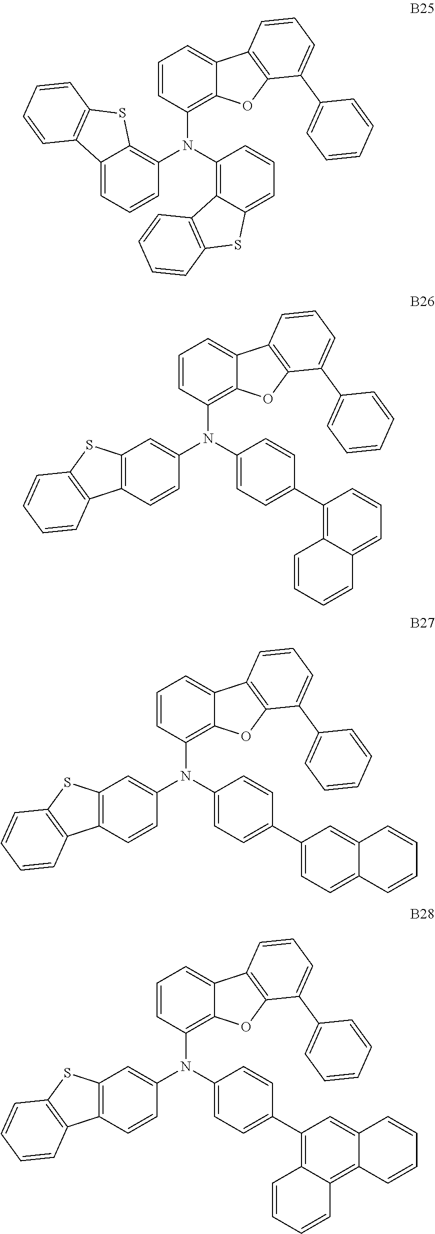 [SMILES: c1ccc(-c2cccc3c2oc2c(N(c4cccc5c4sc4ccccc45)c4cccc5sc6ccccc6c45)cccc23)cc1 ; c1ccc(-c2cccc3c2oc2c(N(c4ccc(-c5cccc6ccccc56)cc4)c4ccc5c(c4)sc4ccccc45)cccc23)cc1 ; c1ccc(-c2cccc3c2oc2c(N(c4ccc(-c5ccc6ccccc6c5)cc4)c4ccc5c(c4)sc4ccccc45)cccc23)cc1 ; c1ccc(-c2cccc3c2oc2c(N(c4ccc(-c5cc6ccccc6c6ccccc56)cc4)c4ccc5c(c4)sc4ccccc45)cccc23)cc1]

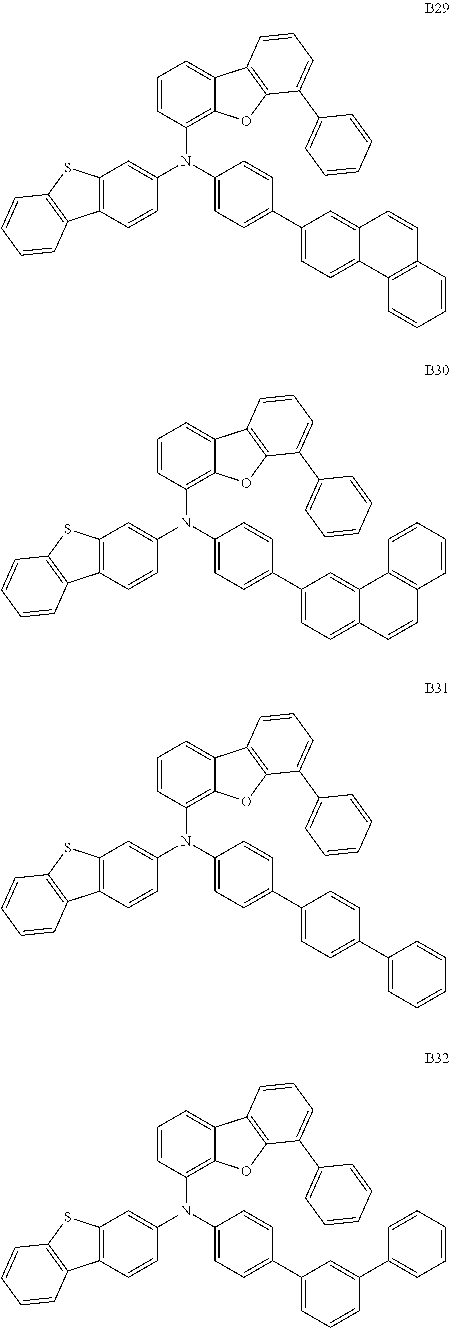 [SMILES: c1ccc(-c2cccc3c2oc2c(N(c4ccc(-c5ccc6c(ccc7ccccc76)c5)cc4)c4ccc5c(c4)sc4ccccc45)cccc23)cc1 ; c1ccc(-c2cccc3c2oc2c(N(c4ccc(-c5ccc6ccc7ccccc7c6c5)cc4)c4ccc5c(c4)sc4ccccc45)cccc23)cc1 ; c1ccc(-c2ccc(-c3ccc(N(c4ccc5c(c4)sc4ccccc45)c4cccc5c4oc4c(-c6ccccc6)cccc45)cc3)cc2)cc1 ; c1ccc(-c2cccc(-c3ccc(N(c4ccc5c(c4)sc4ccccc45)c4cccc5c4oc4c(-c6ccccc6)cccc45)cc3)c2)cc1]

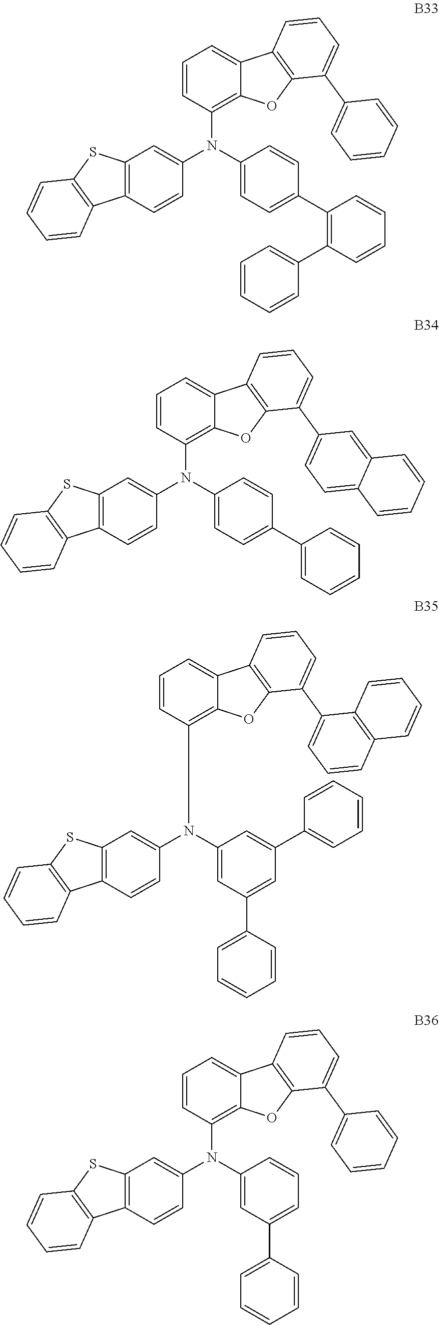

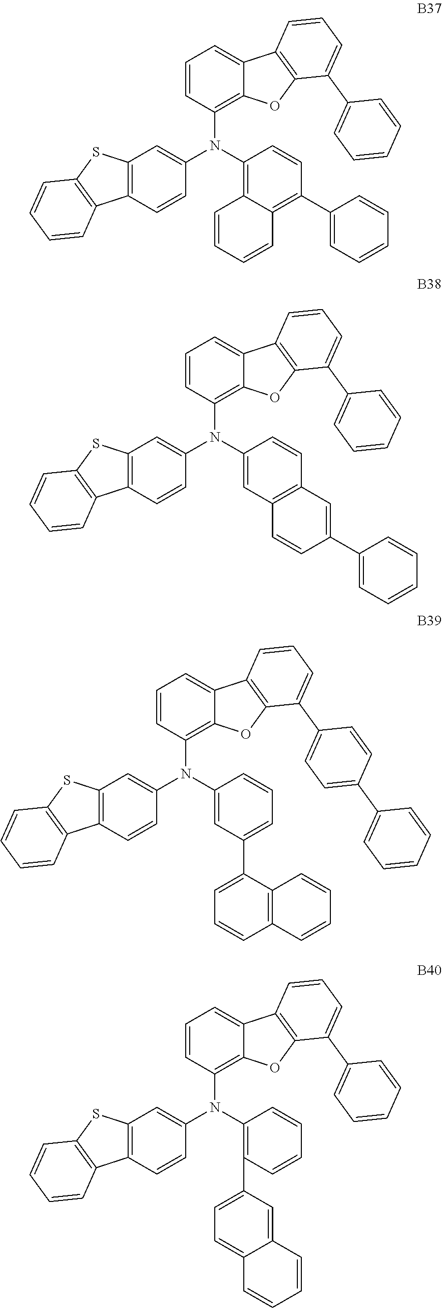

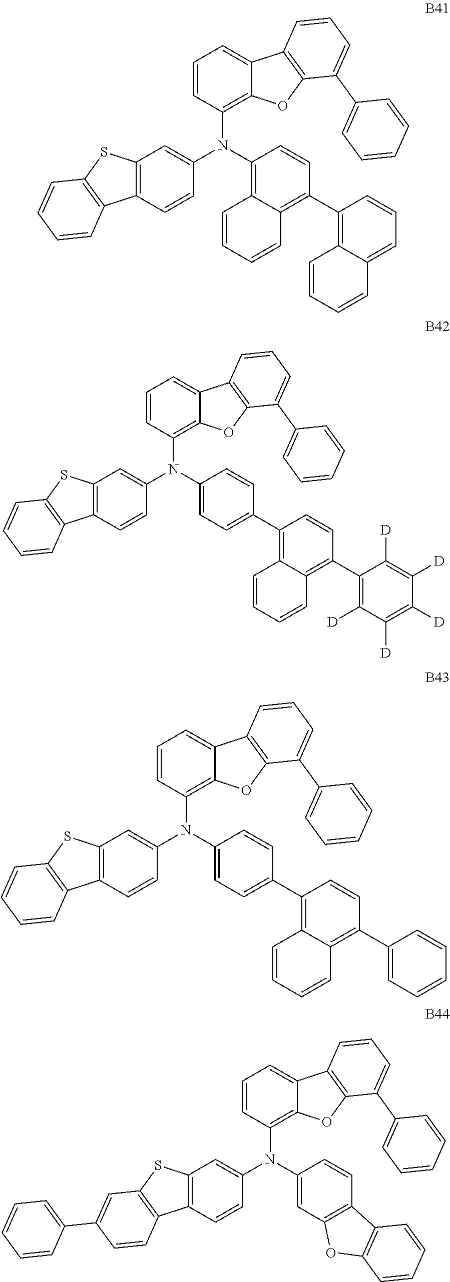 [SMILES: c1ccc(-c2cccc3c2oc2c(N(c4ccc5c(c4)sc4ccccc45)c4ccc(-c5cccc6ccccc56)c5ccccc45)cccc23)cc1 ; [2H]c1c([2H])c([2H])c(-c2ccc(-c3ccc(N(c4ccc5c(c4)sc4ccccc45)c4cccc5c4oc4c(-c6ccccc6)cccc45)cc3)c3ccccc23)c([2H])c1[2H] ; c1ccc(-c2ccc(-c3ccc(N(c4ccc5c(c4)sc4ccccc45)c4cccc5c4oc4c(-c6ccccc6)cccc45)cc3)c3ccccc23)cc1 ; c1ccc(-c2ccc3c(c2)sc2cc(N(c4ccc5c(c4)oc4ccccc45)c4cccc5c4oc4c(-c6ccccc6)cccc45)ccc23)cc1]

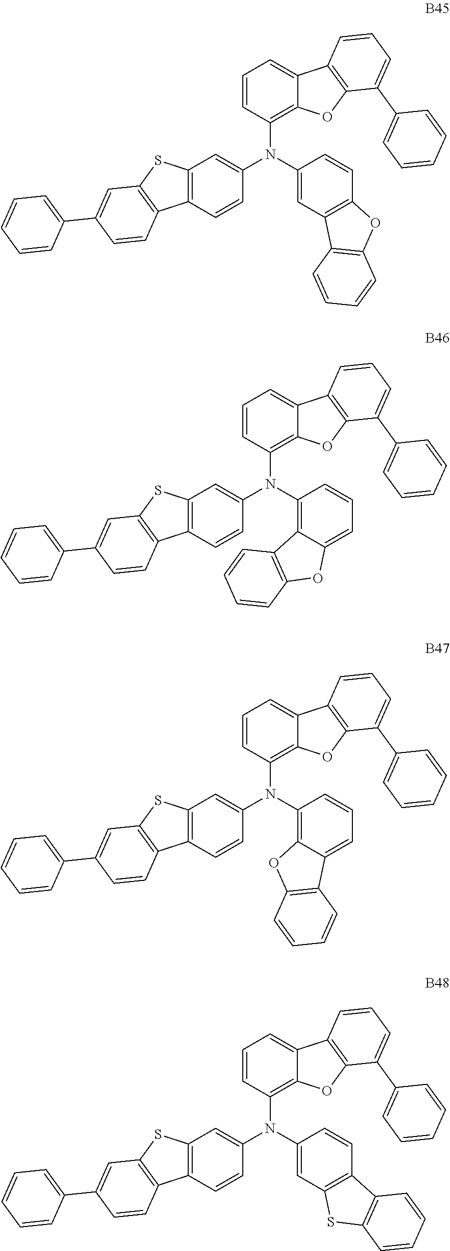 [SMILES: c1ccc(-c2ccc3c(c2)sc2cc(N(c4ccc5oc6ccccc6c5c4)c4cccc5c4oc4c(-c6ccccc6)cccc45)ccc23)cc1 ; c1ccc(-c2ccc3c(c2)sc2cc(N(c4cccc5c4oc4c(-c6ccccc6)cccc45)c4cccc5oc6ccccc6c45)ccc23)cc1 ; c1ccc(-c2ccc3c(c2)sc2cc(N(c4cccc5c4oc4ccccc45)c4cccc5c4oc4c(-c6ccccc6)cccc45)ccc23)cc1 ; c1ccc(-c2ccc3c(c2)sc2cc(N(c4ccc5c(c4)sc4ccccc45)c4cccc5c4oc4c(-c6ccccc6)cccc45)ccc23)cc1]

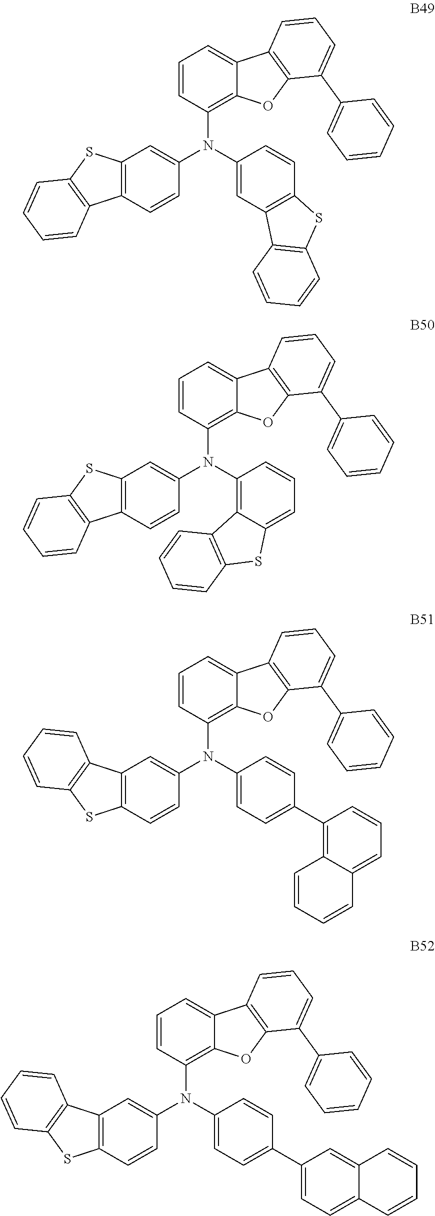

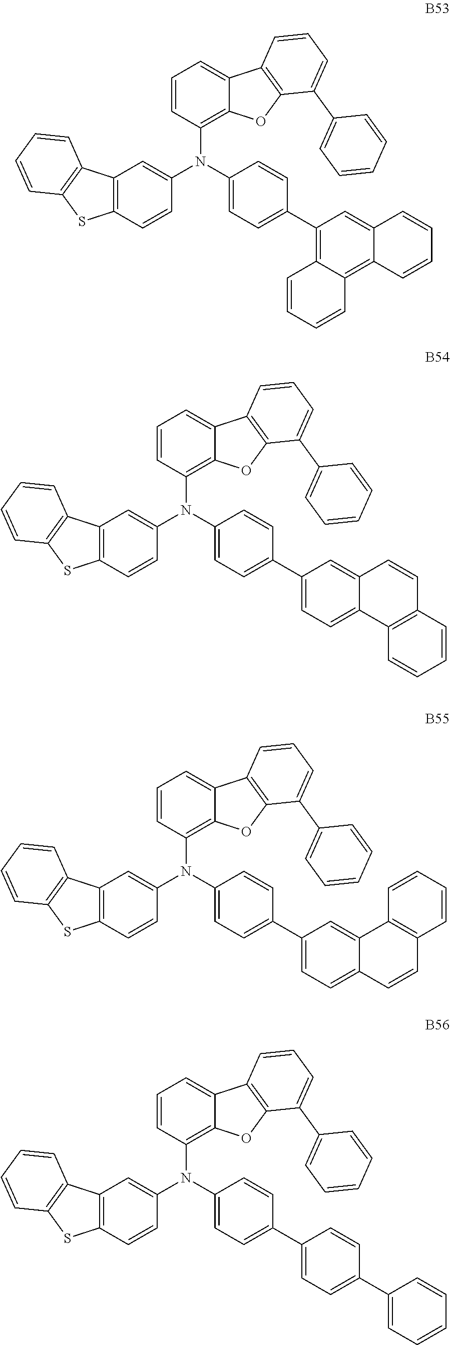 [SMILES: c1ccc(-c2cccc3c2oc2c(N(c4ccc(-c5cc6ccccc6c6ccccc56)cc4)c4ccc5sc6ccccc6c5c4)cccc23)cc1 ; c1ccc(-c2cccc3c2oc2c(N(c4ccc(-c5ccc6c(ccc7ccccc76)c5)cc4)c4ccc5sc6ccccc6c5c4)cccc23)cc1 ; c1ccc(-c2cccc3c2oc2c(N(c4ccc(-c5ccc6ccc7ccccc7c6c5)cc4)c4ccc5sc6ccccc6c5c4)cccc23)cc1 ; c1ccc(-c2ccc(-c3ccc(N(c4ccc5sc6ccccc6c5c4)c4cccc5c4oc4c(-c6ccccc6)cccc45)cc3)cc2)cc1]

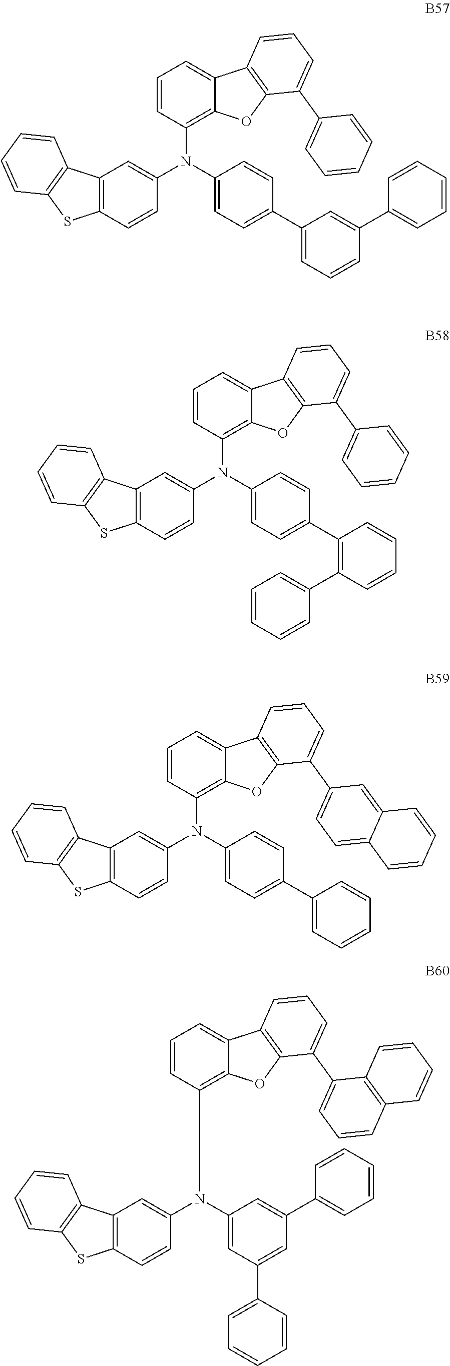 [SMILES: c1ccc(-c2cccc(-c3ccc(N(c4ccc5sc6ccccc6c5c4)c4cccc5c4oc4c(-c6ccccc6)cccc45)cc3)c2)cc1 ; c1ccc(-c2ccccc2-c2ccc(N(c3ccc4sc5ccccc5c4c3)c3cccc4c3oc3c(-c5ccccc5)cccc34)cc2)cc1 ; c1ccc(-c2ccc(N(c3ccc4sc5ccccc5c4c3)c3cccc4c3oc3c(-c5ccc6ccccc6c5)cccc34)cc2)cc1 ; c1ccc(-c2cc(-c3ccccc3)cc(N(c3ccc4sc5ccccc5c4c3)c3cccc4c3oc3c(-c5cccc6ccccc56)cccc34)c2)cc1]

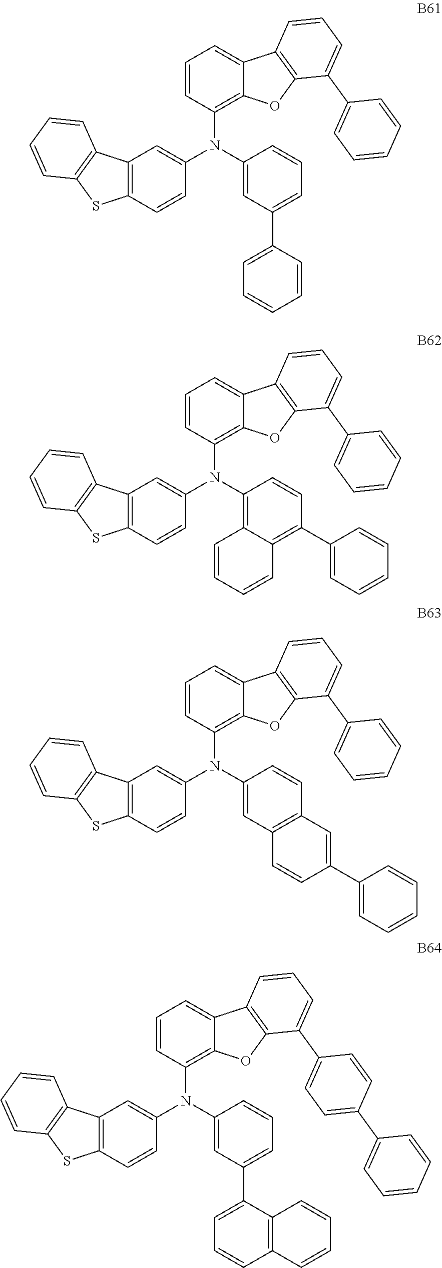

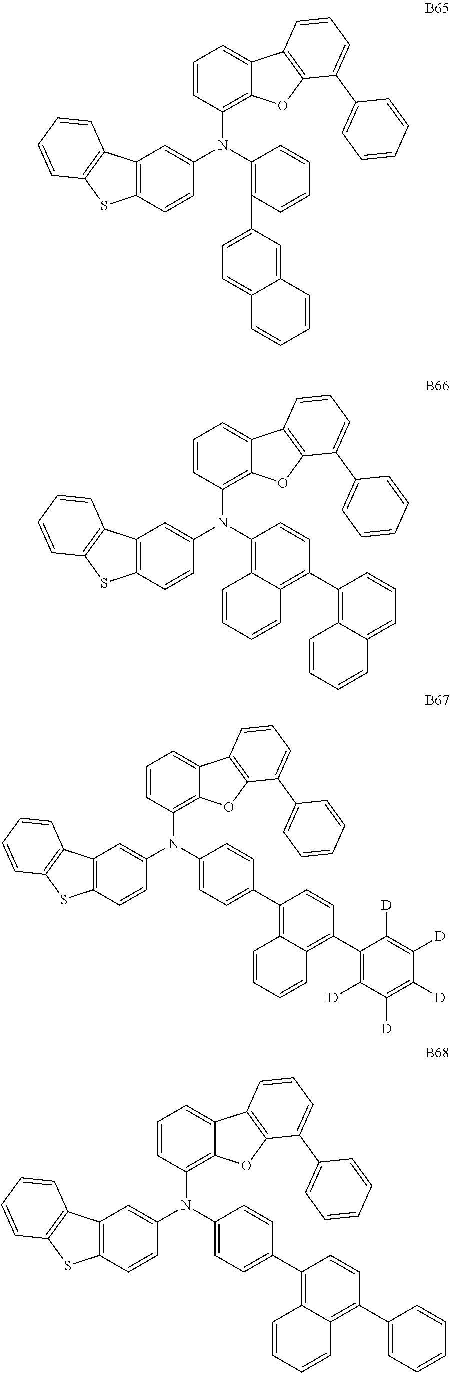 [SMILES: c1ccc(-c2cccc3c2oc2c(N(c4ccc5sc6ccccc6c5c4)c4ccccc4-c4ccc5ccccc5c4)cccc23)cc1 ; c1ccc(-c2cccc3c2oc2c(N(c4ccc5sc6ccccc6c5c4)c4ccc(-c5cccc6ccccc56)c5ccccc45)cccc23)cc1 ; [2H]c1c([2H])c([2H])c(-c2ccc(-c3ccc(N(c4ccc5sc6ccccc6c5c4)c4cccc5c4oc4c(-c6ccccc6)cccc45)cc3)c3ccccc23)c([2H])c1[2H] ; c1ccc(-c2ccc(-c3ccc(N(c4ccc5sc6ccccc6c5c4)c4cccc5c4oc4c(-c6ccccc6)cccc45)cc3)c3ccccc23)cc1]

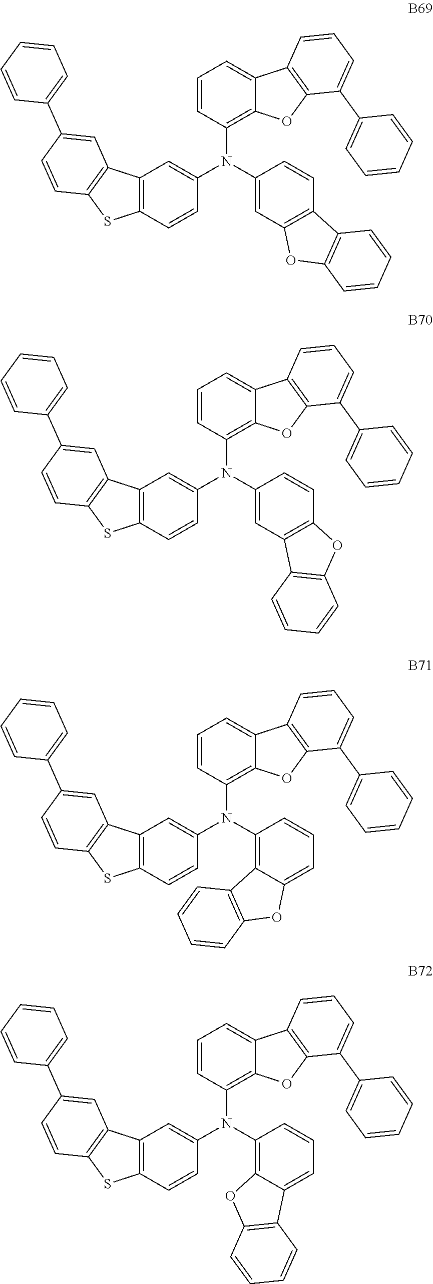 [SMILES: c1ccc(-c2ccc3sc4ccc(N(c5ccc6c(c5)oc5ccccc56)c5cccc6c5oc5c(-c7ccccc7)cccc56)cc4c3c2)cc1 ; c1ccc(-c2ccc3sc4ccc(N(c5ccc6oc7ccccc7c6c5)c5cccc6c5oc5c(-c7ccccc7)cccc56)cc4c3c2)cc1 ; c1ccc(-c2ccc3sc4ccc(N(c5cccc6c5oc5c(-c7ccccc7)cccc56)c5cccc6oc7ccccc7c56)cc4c3c2)cc1 ; c1ccc(-c2ccc3sc4ccc(N(c5cccc6c5oc5ccccc56)c5cccc6c5oc5c(-c7ccccc7)cccc56)cc4c3c2)cc1]

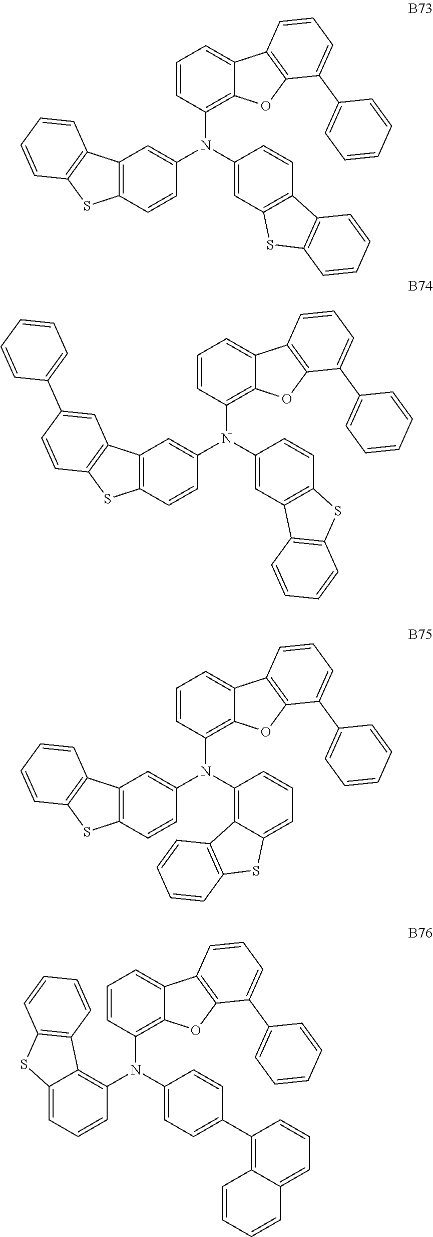

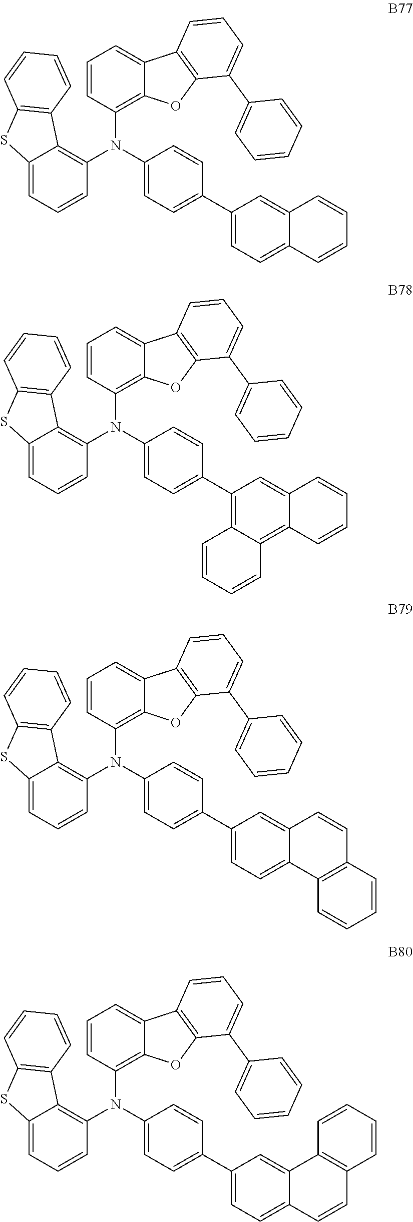

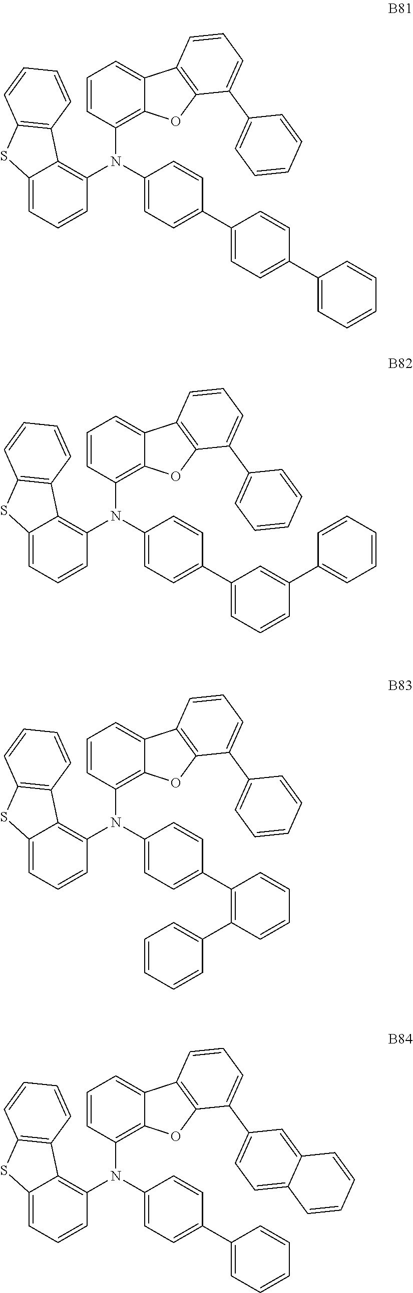 [SMILES: c1ccc(-c2ccc(-c3ccc(N(c4cccc5c4oc4c(-c6ccccc6)cccc45)c4cccc5sc6ccccc6c45)cc3)cc2)cc1 ; c1ccc(-c2cccc(-c3ccc(N(c4cccc5c4oc4c(-c6ccccc6)cccc45)c4cccc5sc6ccccc6c45)cc3)c2)cc1 ; c1ccc(-c2ccccc2-c2ccc(N(c3cccc4c3oc3c(-c5ccccc5)cccc34)c3cccc4sc5ccccc5c34)cc2)cc1 ; c1ccc(-c2ccc(N(c3cccc4c3oc3c(-c5ccc6ccccc6c5)cccc34)c3cccc4sc5ccccc5c34)cc2)cc1]

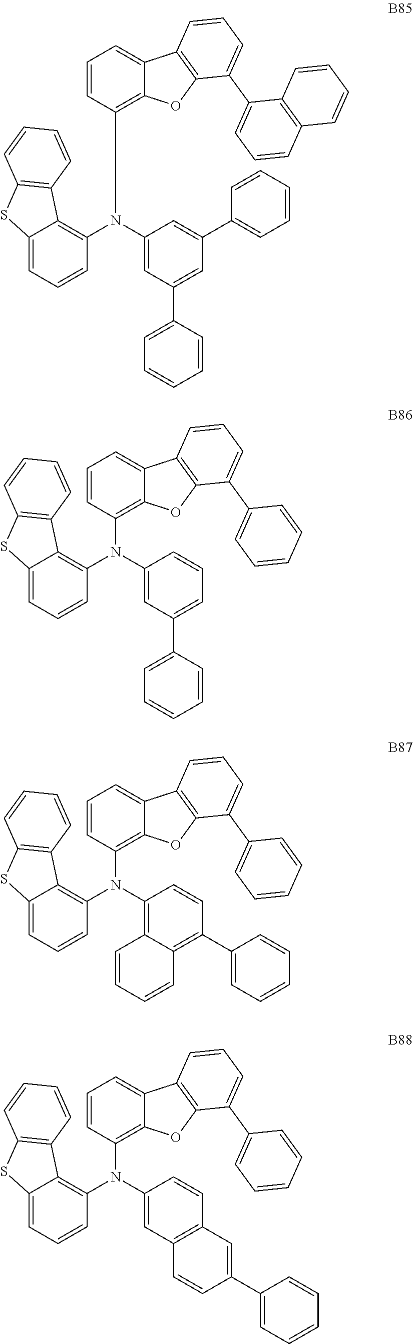

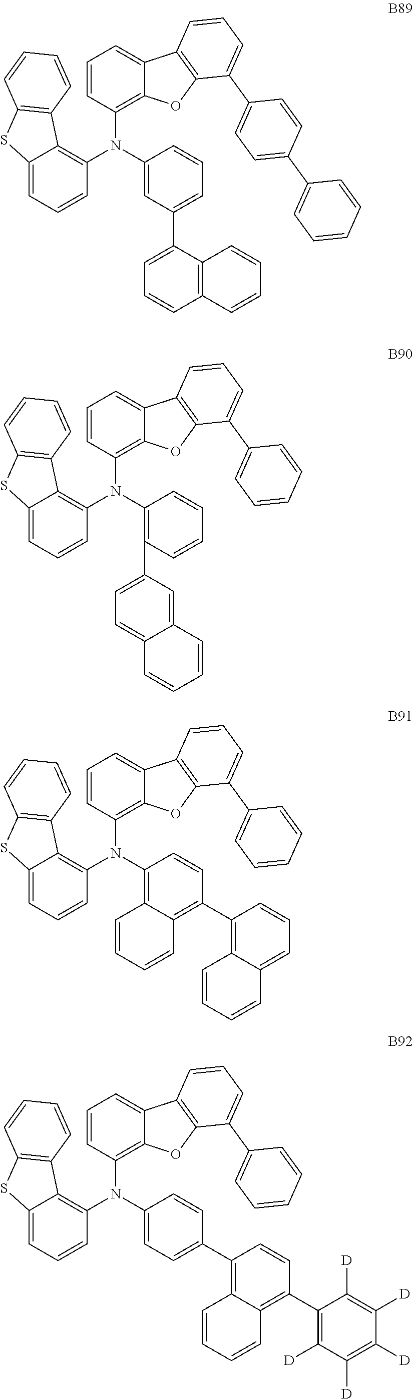

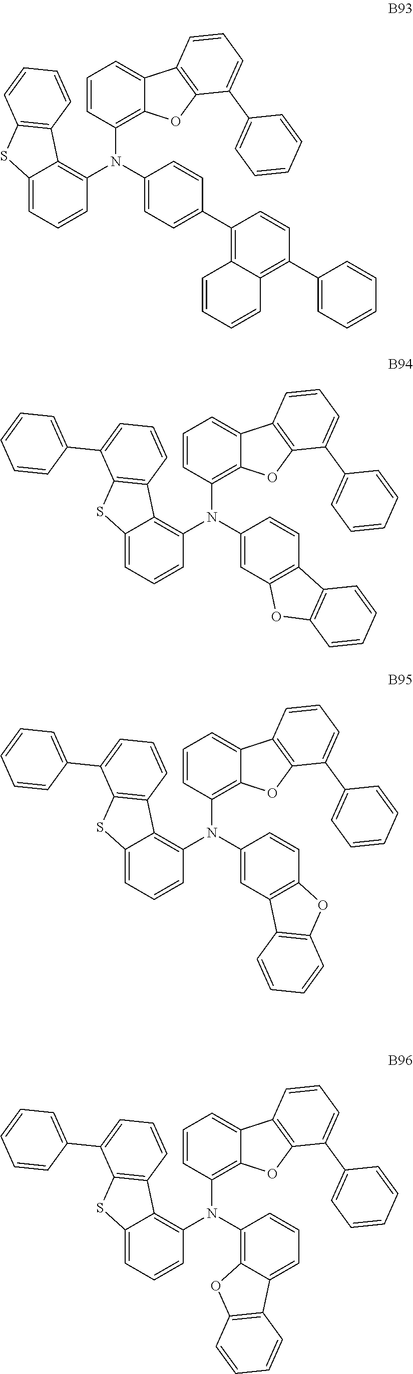 [SMILES: c1ccc(-c2ccc(-c3ccc(N(c4cccc5c4oc4c(-c6ccccc6)cccc45)c4cccc5sc6ccccc6c45)cc3)c3ccccc23)cc1 ; c1ccc(-c2cccc3c2oc2c(N(c4ccc5c(c4)oc4ccccc45)c4cccc5sc6c(-c7ccccc7)cccc6c45)cccc23)cc1 ; c1ccc(-c2cccc3c2oc2c(N(c4ccc5oc6ccccc6c5c4)c4cccc5sc6c(-c7ccccc7)cccc6c45)cccc23)cc1 ; c1ccc(-c2cccc3c2oc2c(N(c4cccc5c4oc4ccccc45)c4cccc5sc6c(-c7ccccc7)cccc6c45)cccc23)cc1]

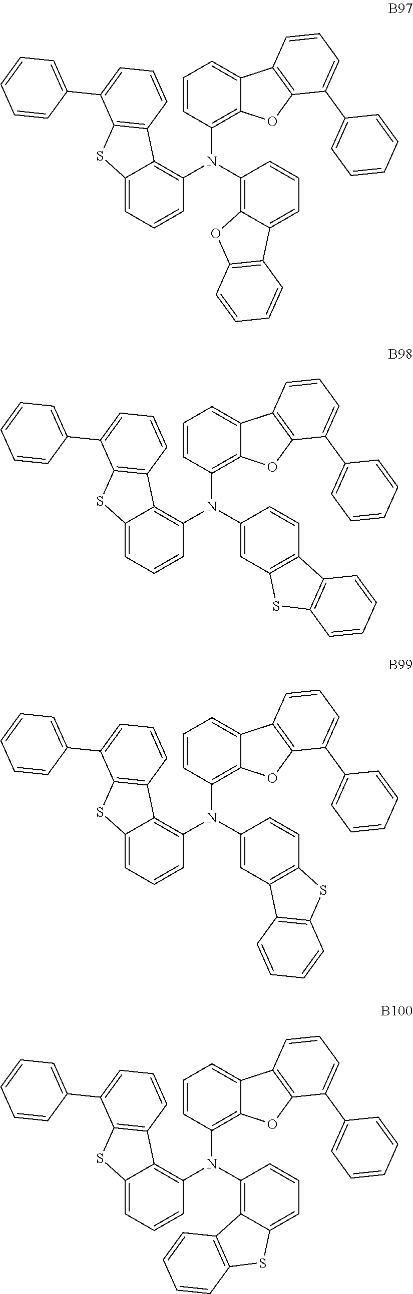 [SMILES: c1ccc(-c2cccc3c2oc2c(N(c4cccc5c4oc4ccccc45)c4cccc5sc6c(-c7ccccc7)cccc6c45)cccc23)cc1 ; c1ccc(-c2cccc3c2oc2c(N(c4ccc5c(c4)sc4ccccc45)c4cccc5sc6c(-c7ccccc7)cccc6c45)cccc23)cc1 ; c1ccc(-c2cccc3c2oc2c(N(c4ccc5sc6ccccc6c5c4)c4cccc5sc6c(-c7ccccc7)cccc6c45)cccc23)cc1 ; c1ccc(-c2cccc3c2oc2c(N(c4cccc5sc6ccccc6c45)c4cccc5sc6c(-c7ccccc7)cccc6c45)cccc23)cc1]

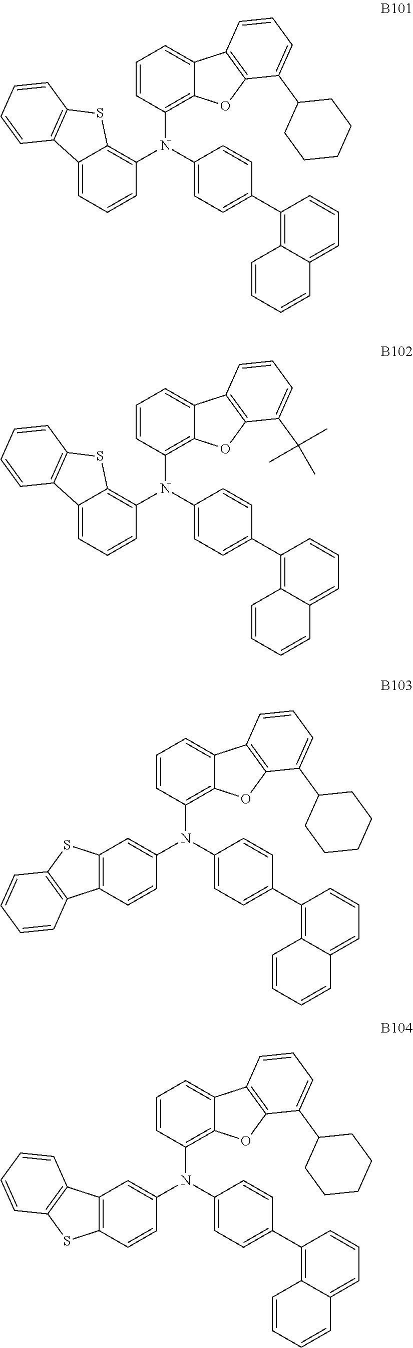 [SMILES: c1ccc2c(-c3ccc(N(c4cccc5c4oc4c(C6CCCCC6)cccc45)c4cccc5c4sc4ccccc45)cc3)cccc2c1 ; CC(C)(C)c1cccc2c1oc1c(N(c3ccc(-c4cccc5ccccc45)cc3)c3cccc4c3sc3ccccc34)cccc12 ; c1ccc2c(-c3ccc(N(c4ccc5c(c4)sc4ccccc45)c4cccc5c4oc4c(C6CCCCC6)cccc45)cc3)cccc2c1 ; c1ccc2c(-c3ccc(N(c4ccc5sc6ccccc6c5c4)c4cccc5c4oc4c(C6CCCCC6)cccc45)cc3)cccc2c1]

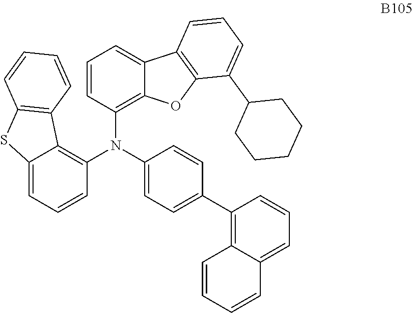 [SMILES: c1ccc2c(-c3ccc(N(c4cccc5c4oc4c(C6CCCCC6)cccc45)c4cccc5sc6ccccc6c45)cc3)cccc2c1]

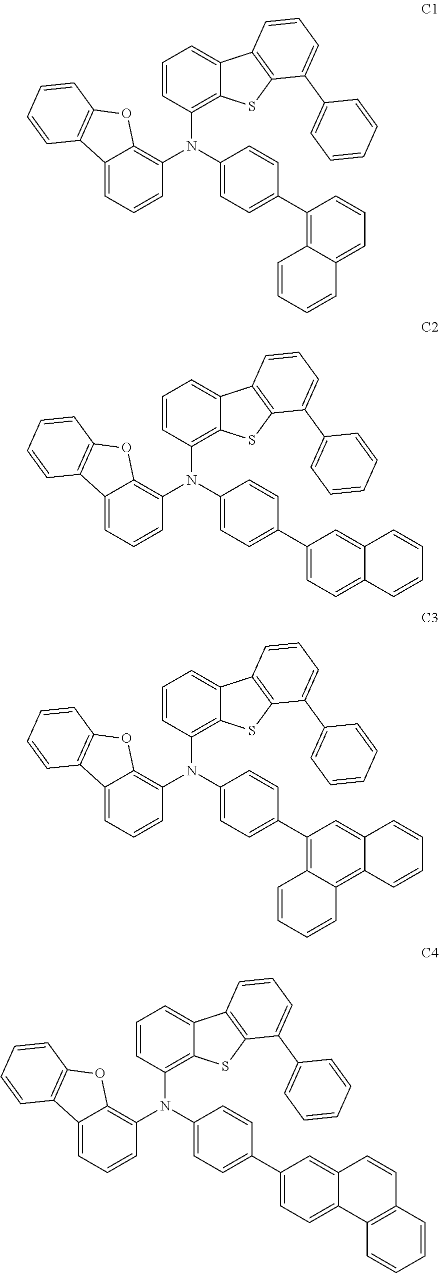

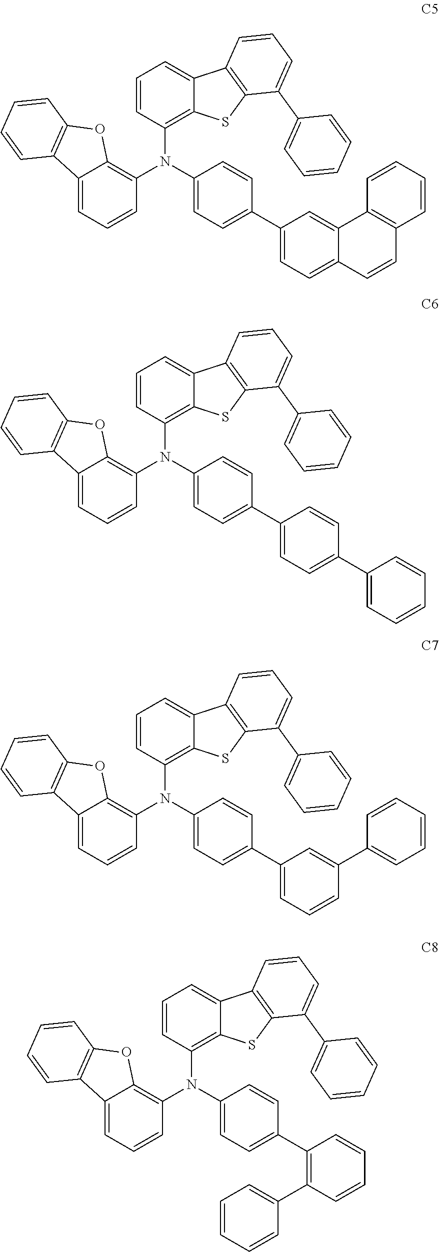

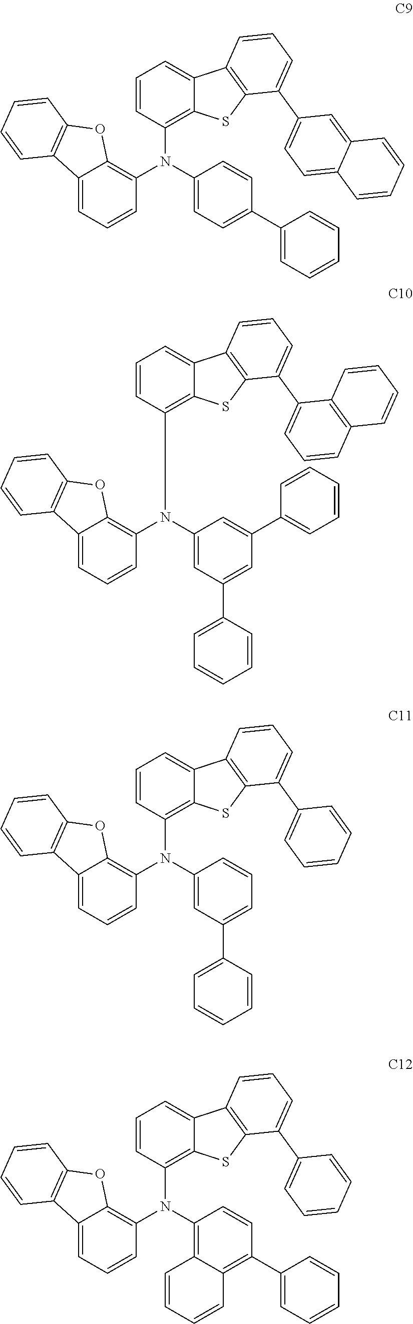 [SMILES: c1ccc(-c2ccc(N(c3cccc4c3oc3ccccc34)c3cccc4c3sc3c(-c5ccc6ccccc6c5)cccc34)cc2)cc1 ; c1ccc(-c2cc(-c3ccccc3)cc(N(c3cccc4c3oc3ccccc34)c3cccc4c3sc3c(-c5cccc6ccccc56)cccc34)c2)cc1 ; c1ccc(-c2cccc(N(c3cccc4c3oc3ccccc34)c3cccc4c3sc3c(-c5ccccc5)cccc34)c2)cc1 ; c1ccc(-c2ccc(N(c3cccc4c3oc3ccccc34)c3cccc4c3sc3c(-c5ccccc5)cccc34)c3ccccc23)cc1]

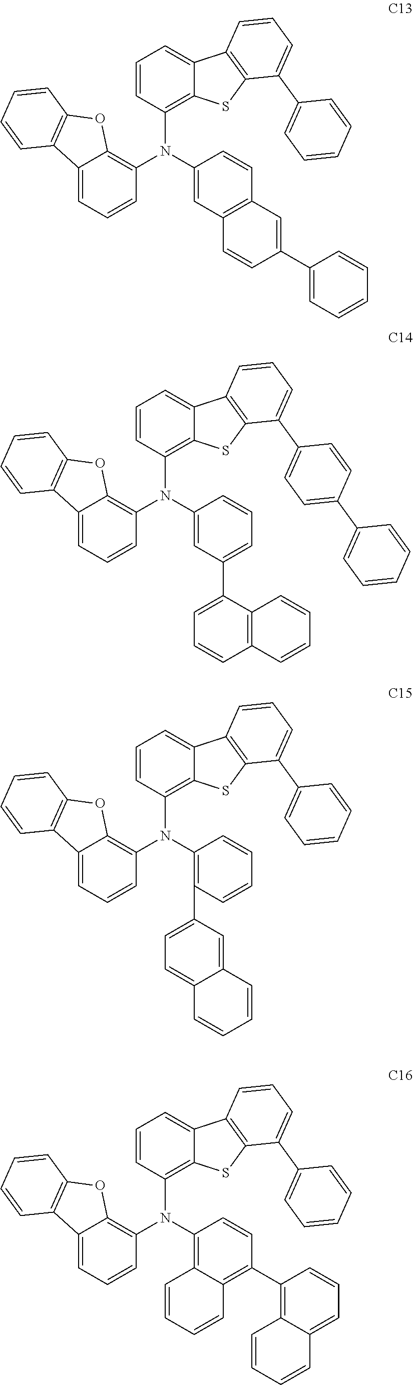

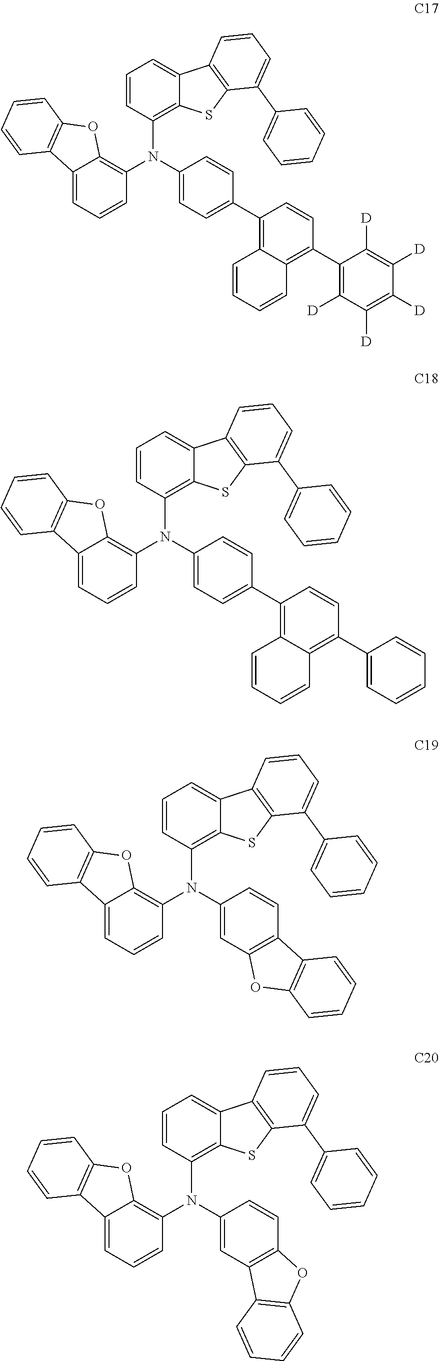 [SMILES: [2H]c1c([2H])c([2H])c(-c2ccc(-c3ccc(N(c4cccc5c4oc4ccccc45)c4cccc5c4sc4c(-c6ccccc6)cccc45)cc3)c3ccccc23)c([2H])c1[2H] ; c1ccc(-c2ccc(-c3ccc(N(c4cccc5c4oc4ccccc45)c4cccc5c4sc4c(-c6ccccc6)cccc45)cc3)c3ccccc23)cc1 ; c1ccc(-c2cccc3c2sc2c(N(c4ccc5c(c4)oc4ccccc45)c4cccc5c4oc4ccccc45)cccc23)cc1 ; c1ccc(-c2cccc3c2sc2c(N(c4ccc5oc6ccccc6c5c4)c4cccc5c4oc4ccccc45)cccc23)cc1]

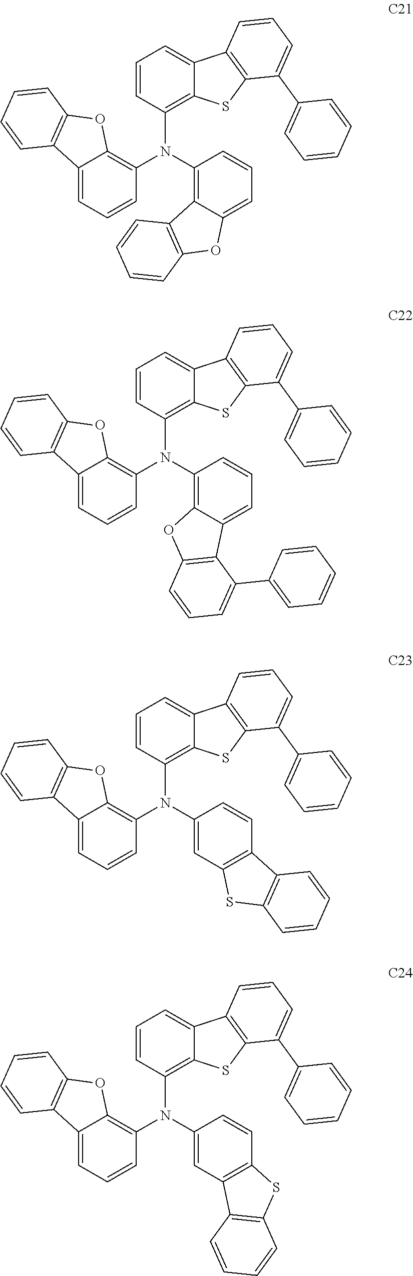 [SMILES: c1ccc(-c2cccc3c2sc2c(N(c4cccc5c4oc4ccccc45)c4cccc5oc6ccccc6c45)cccc23)cc1 ; c1ccc(-c2cccc3c2sc2c(N(c4cccc5c4oc4ccccc45)c4cccc5c4oc4cccc(-c6ccccc6)c45)cccc23)cc1 ; c1ccc(-c2cccc3c2sc2c(N(c4ccc5c(c4)sc4ccccc45)c4cccc5c4oc4ccccc45)cccc23)cc1 ; c1ccc(-c2cccc3c2sc2c(N(c4ccc5sc6ccccc6c5c4)c4cccc5c4oc4ccccc45)cccc23)cc1]

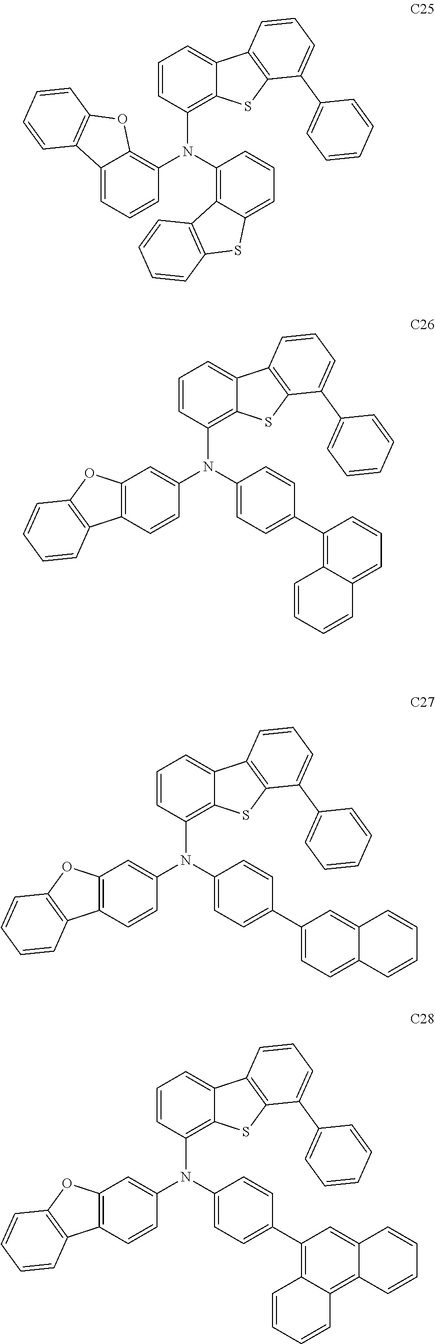 [SMILES: c1ccc(-c2cccc3c2sc2c(N(c4cccc5c4oc4ccccc45)c4cccc5sc6ccccc6c45)cccc23)cc1 ; c1ccc(-c2cccc3c2sc2c(N(c4ccc(-c5cccc6ccccc56)cc4)c4ccc5c(c4)oc4ccccc45)cccc23)cc1 ; c1ccc(-c2cccc3c2sc2c(N(c4ccc(-c5ccc6ccccc6c5)cc4)c4ccc5c(c4)oc4ccccc45)cccc23)cc1 ; c1ccc(-c2cccc3c2sc2c(N(c4ccc(-c5cc6ccccc6c6ccccc56)cc4)c4ccc5c(c4)oc4ccccc45)cccc23)cc1]

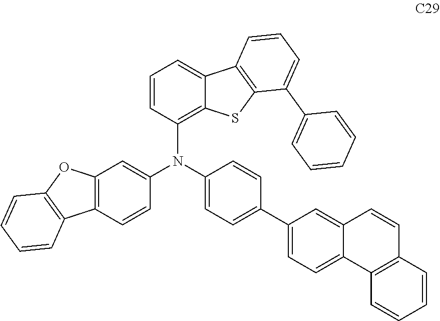 [SMILES: c1ccc(-c2cccc3c2sc2c(N(c4ccc(-c5ccc6c(ccc7ccccc76)c5)cc4)c4ccc5c(c4)oc4ccccc45)cccc23)cc1]

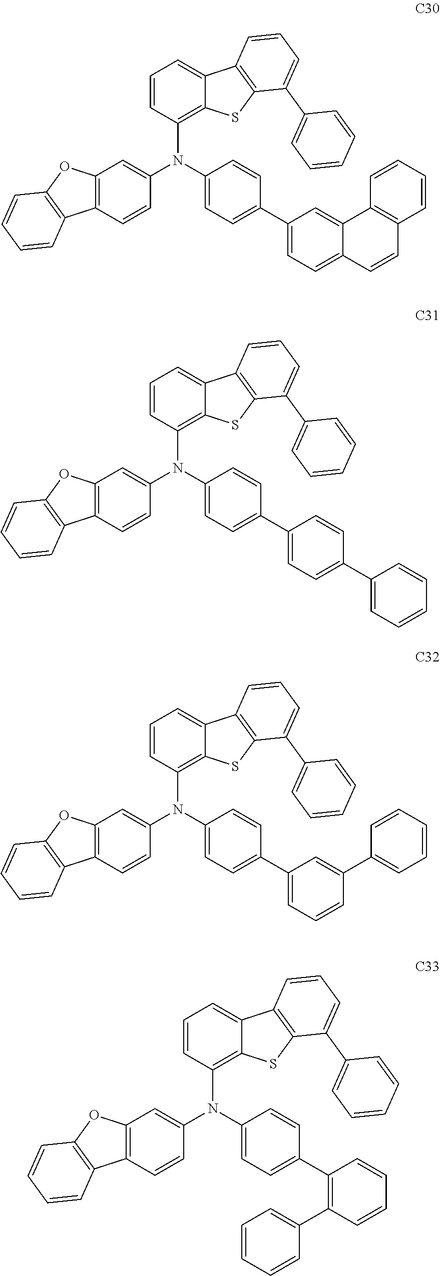 [SMILES: c1ccc(-c2cccc3c2sc2c(N(c4ccc(-c5ccc6ccc7ccccc7c6c5)cc4)c4ccc5c(c4)oc4ccccc45)cccc23)cc1 ; c1ccc(-c2ccc(-c3ccc(N(c4ccc5c(c4)oc4ccccc45)c4cccc5c4sc4c(-c6ccccc6)cccc45)cc3)cc2)cc1 ; c1ccc(-c2cccc(-c3ccc(N(c4ccc5c(c4)oc4ccccc45)c4cccc5c4sc4c(-c6ccccc6)cccc45)cc3)c2)cc1 ; c1ccc(-c2ccccc2-c2ccc(N(c3ccc4c(c3)oc3ccccc34)c3cccc4c3sc3c(-c5ccccc5)cccc34)cc2)cc1]

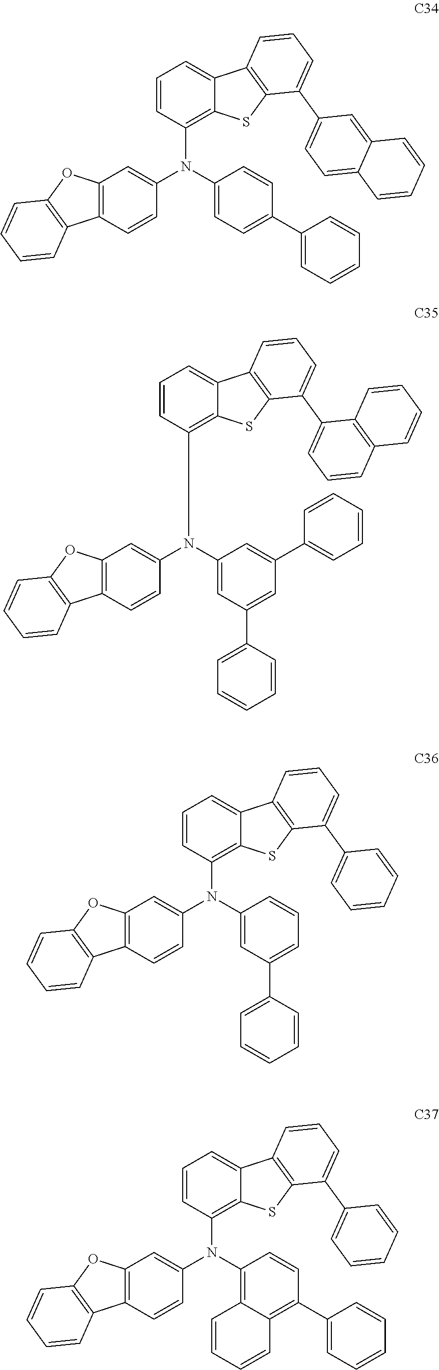 [SMILES: c1ccc(-c2ccc(N(c3ccc4c(c3)oc3ccccc34)c3cccc4c3sc3c(-c5ccc6ccccc6c5)cccc34)cc2)cc1 ; c1ccc(-c2cc(-c3ccccc3)cc(N(c3ccc4c(c3)oc3ccccc34)c3cccc4c3sc3c(-c5cccc6ccccc56)cccc34)c2)cc1 ; c1ccc(-c2cccc(N(c3ccc4c(c3)oc3ccccc34)c3cccc4c3sc3c(-c5ccccc5)cccc34)c2)cc1 ; c1ccc(-c2ccc(N(c3ccc4c(c3)oc3ccccc34)c3cccc4c3sc3c(-c5ccccc5)cccc34)c3ccccc23)cc1]

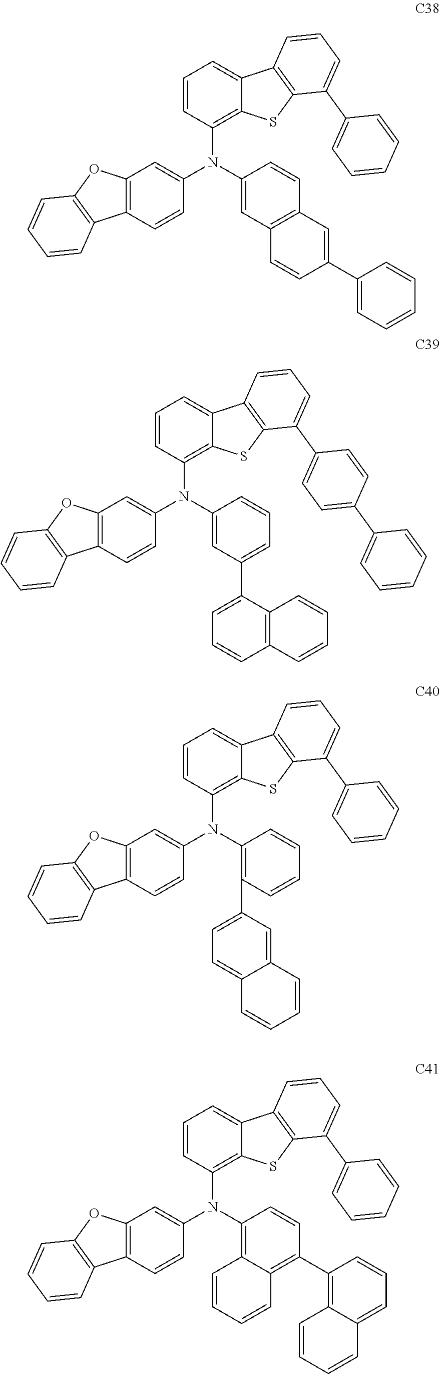

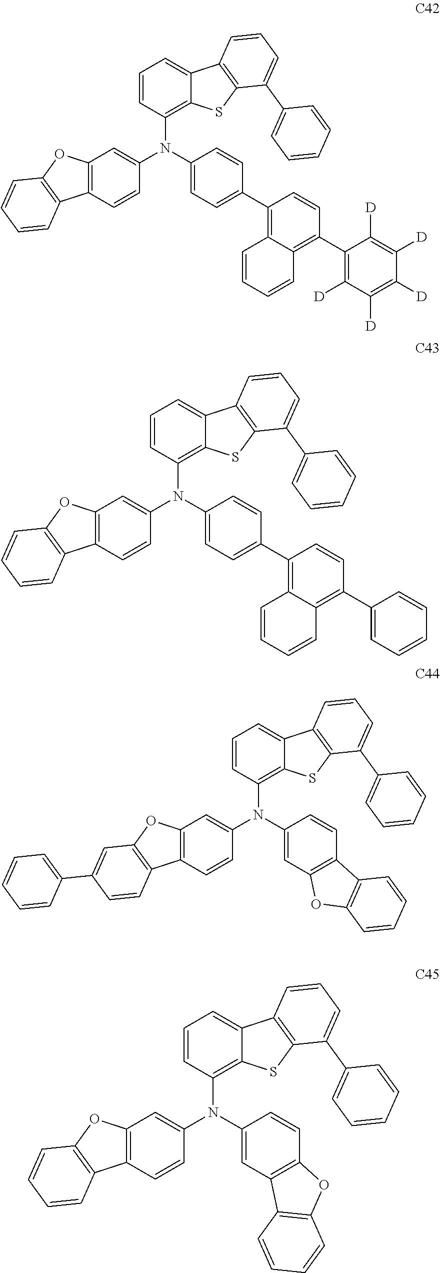 [SMILES: [2H]c1c([2H])c([2H])c(-c2ccc(-c3ccc(N(c4ccc5c(c4)oc4ccccc45)c4cccc5c4sc4c(-c6ccccc6)cccc45)cc3)c3ccccc23)c([2H])c1[2H] ; c1ccc(-c2ccc(-c3ccc(N(c4ccc5c(c4)oc4ccccc45)c4cccc5c4sc4c(-c6ccccc6)cccc45)cc3)c3ccccc23)cc1 ; c1ccc(-c2ccc3c(c2)oc2cc(N(c4ccc5c(c4)oc4ccccc45)c4cccc5c4sc4c(-c6ccccc6)cccc45)ccc23)cc1 ; c1ccc(-c2cccc3c2sc2c(N(c4ccc5c(c4)oc4ccccc45)c4ccc5oc6ccccc6c5c4)cccc23)cc1]

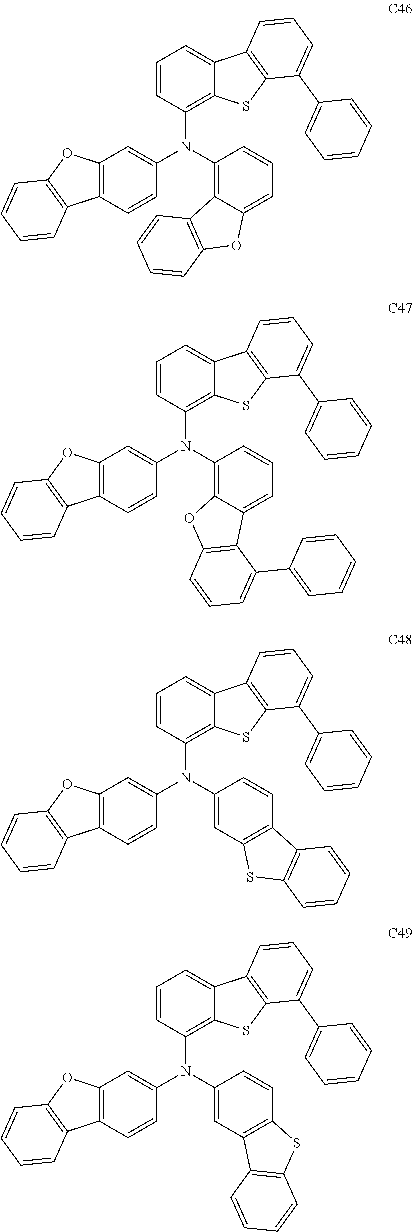

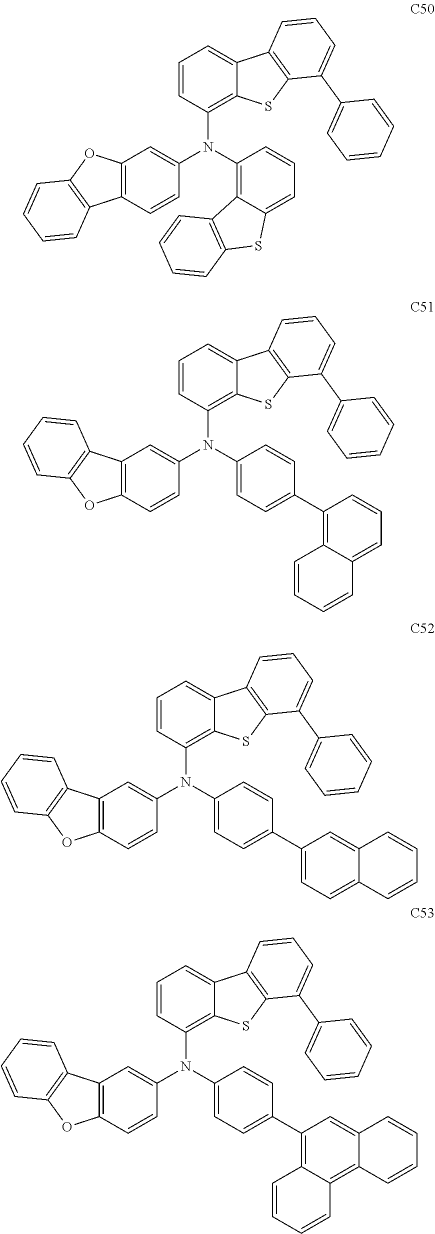 [SMILES: c1ccc(-c2cccc3c2sc2c(N(c4ccc5c(c4)oc4ccccc45)c4cccc5sc6ccccc6c45)cccc23)cc1 ; c1ccc(-c2cccc3c2sc2c(N(c4ccc(-c5cccc6ccccc56)cc4)c4ccc5oc6ccccc6c5c4)cccc23)cc1 ; c1ccc(-c2cccc3c2sc2c(N(c4ccc(-c5ccc6ccccc6c5)cc4)c4ccc5oc6ccccc6c5c4)cccc23)cc1 ; c1ccc(-c2cccc3c2sc2c(N(c4ccc(-c5cc6ccccc6c6ccccc56)cc4)c4ccc5oc6ccccc6c5c4)cccc23)cc1]

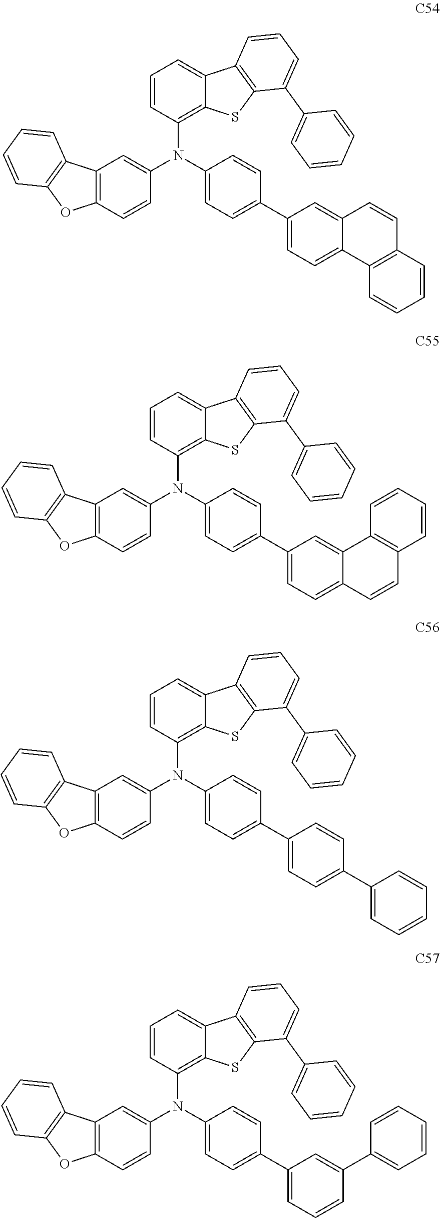 [SMILES: c1ccc(-c2cccc3c2sc2c(N(c4ccc(-c5ccc6c(ccc7ccccc76)c5)cc4)c4ccc5oc6ccccc6c5c4)cccc23)cc1 ; c1ccc(-c2cccc3c2sc2c(N(c4ccc(-c5ccc6ccc7ccccc7c6c5)cc4)c4ccc5oc6ccccc6c5c4)cccc23)cc1 ; c1ccc(-c2ccc(-c3ccc(N(c4ccc5oc6ccccc6c5c4)c4cccc5c4sc4c(-c6ccccc6)cccc45)cc3)cc2)cc1 ; c1ccc(-c2cccc(-c3ccc(N(c4ccc5oc6ccccc6c5c4)c4cccc5c4sc4c(-c6ccccc6)cccc45)cc3)c2)cc1]

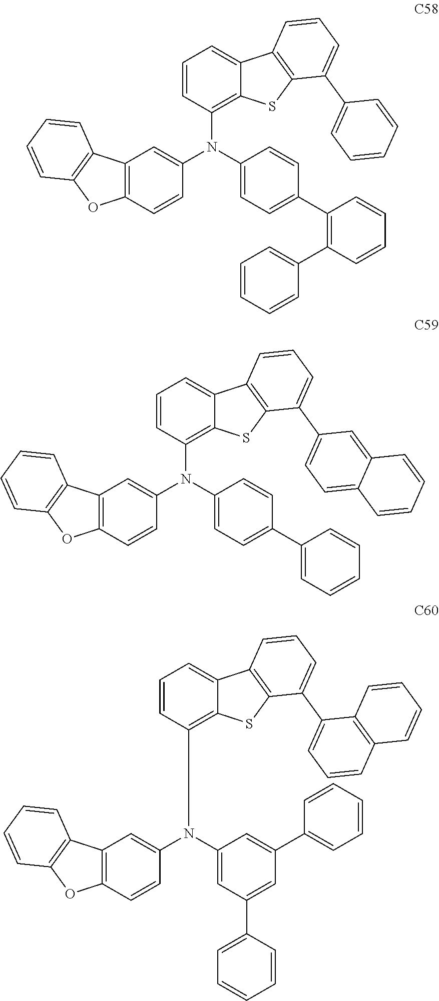

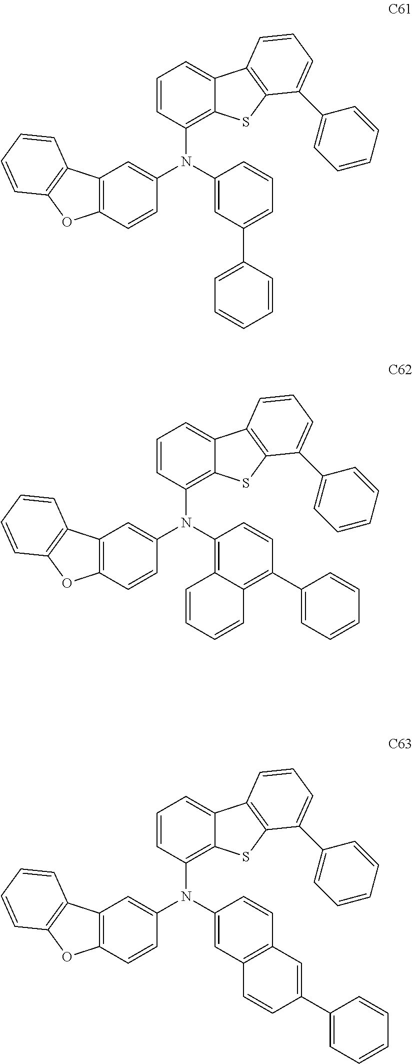 [SMILES: c1ccc(-c2cccc(N(c3ccc4oc5ccccc5c4c3)c3cccc4c3sc3c(-c5ccccc5)cccc34)c2)cc1 ; c1ccc(-c2ccc(N(c3ccc4oc5ccccc5c4c3)c3cccc4c3sc3c(-c5ccccc5)cccc34)c3ccccc23)cc1 ; c1ccc(-c2ccc3cc(N(c4ccc5oc6ccccc6c5c4)c4cccc5c4sc4c(-c6ccccc6)cccc45)ccc3c2)cc1]

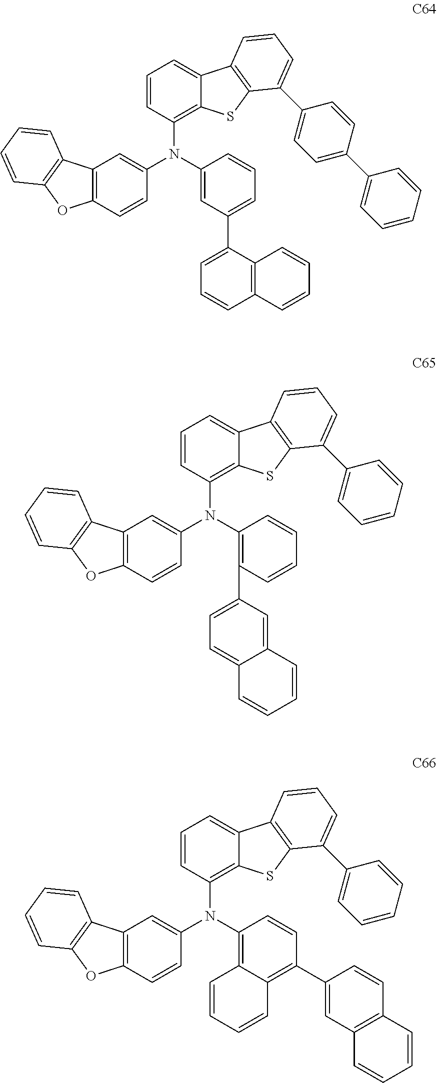 [SMILES: c1ccc(-c2ccc(-c3cccc4c3sc3c(N(c5cccc(-c6cccc7ccccc67)c5)c5ccc6oc7ccccc7c6c5)cccc34)cc2)cc1 ; c1ccc(-c2cccc3c2sc2c(N(c4ccc5oc6ccccc6c5c4)c4ccccc4-c4ccc5ccccc5c4)cccc23)cc1 ; c1ccc(-c2cccc3c2sc2c(N(c4ccc5oc6ccccc6c5c4)c4ccc(-c5ccc6ccccc6c5)c5ccccc45)cccc23)cc1]

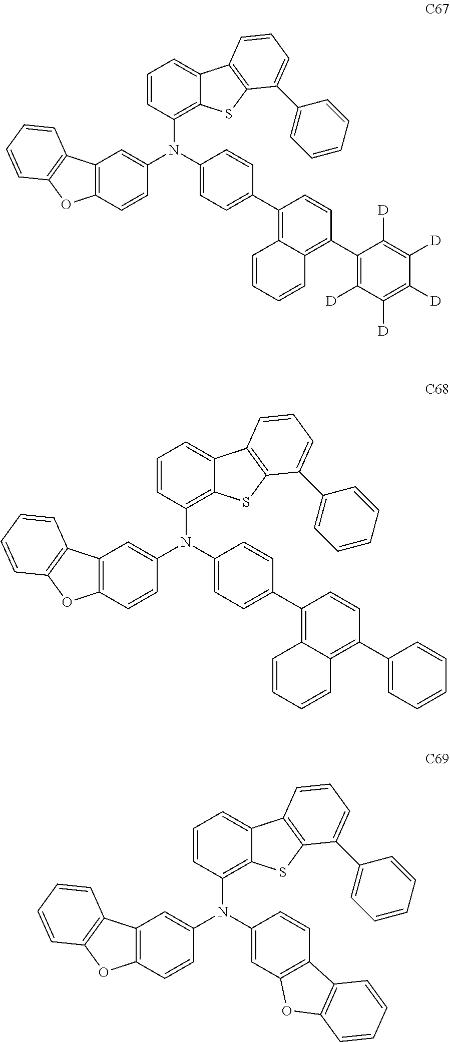 [SMILES: [2H]c1c([2H])c([2H])c(-c2ccc(-c3ccc(N(c4ccc5oc6ccccc6c5c4)c4cccc5c4sc4c(-c6ccccc6)cccc45)cc3)c3ccccc23)c([2H])c1[2H] ; c1ccc(-c2ccc(-c3ccc(N(c4ccc5oc6ccccc6c5c4)c4cccc5c4sc4c(-c6ccccc6)cccc45)cc3)c3ccccc23)cc1 ; c1ccc(-c2cccc3c2sc2c(N(c4ccc5c(c4)oc4ccccc45)c4ccc5oc6ccccc6c5c4)cccc23)cc1]

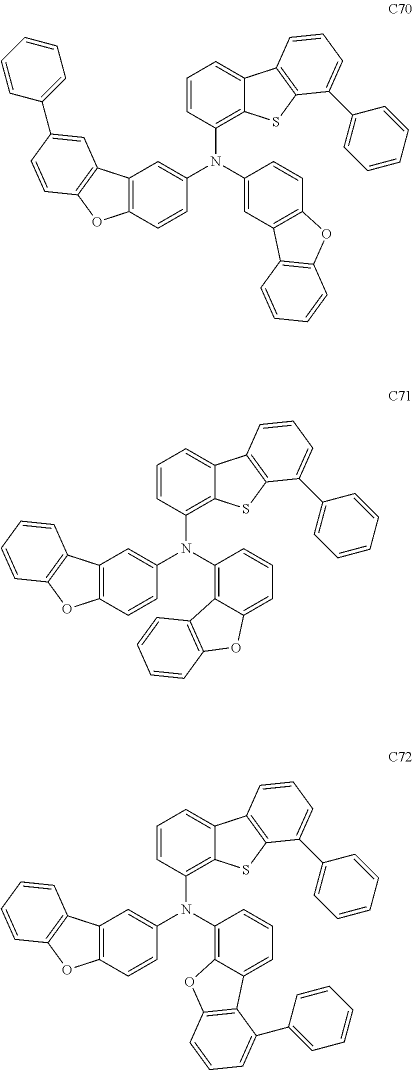 [SMILES: c1ccc(-c2ccc3oc4ccc(N(c5ccc6oc7ccccc7c6c5)c5cccc6c5sc5c(-c7ccccc7)cccc56)cc4c3c2)cc1 ; c1ccc(-c2cccc3c2sc2c(N(c4ccc5oc6ccccc6c5c4)c4cccc5oc6ccccc6c45)cccc23)cc1 ; c1ccc(-c2cccc3c2sc2c(N(c4ccc5oc6ccccc6c5c4)c4cccc5c4oc4cccc(-c6ccccc6)c45)cccc23)cc1]

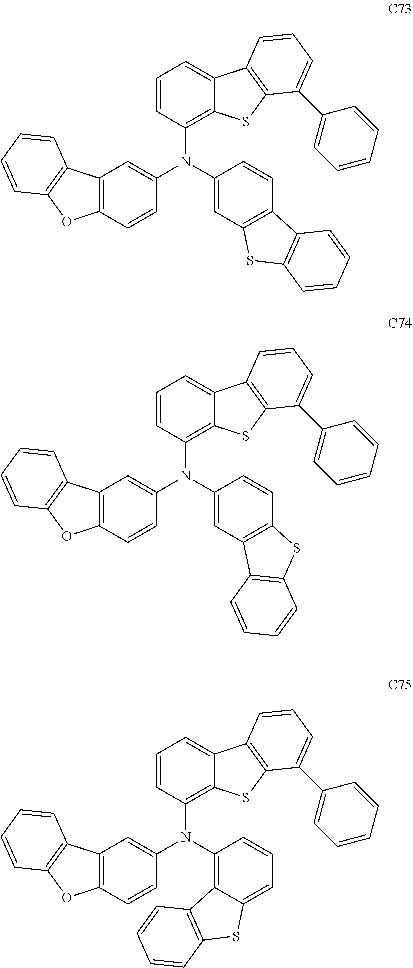 [SMILES: c1ccc(-c2cccc3c2sc2c(N(c4ccc5c(c4)sc4ccccc45)c4ccc5oc6ccccc6c5c4)cccc23)cc1 ; c1ccc(-c2cccc3c2sc2c(N(c4ccc5oc6ccccc6c5c4)c4ccc5sc6ccccc6c5c4)cccc23)cc1 ; c1ccc(-c2cccc3c2sc2c(N(c4ccc5oc6ccccc6c5c4)c4cccc5sc6ccccc6c45)cccc23)cc1]

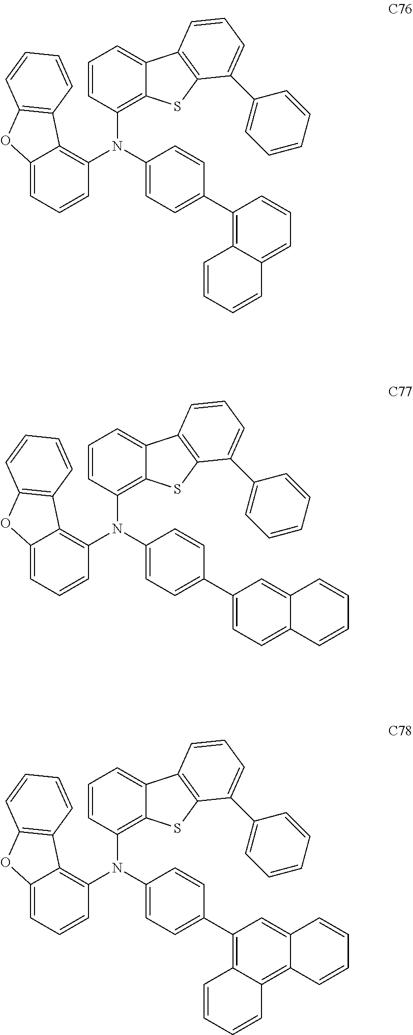 [SMILES: c1ccc(-c2cccc3c2sc2c(N(c4ccc(-c5cccc6ccccc56)cc4)c4cccc5oc6ccccc6c45)cccc23)cc1 ; c1ccc(-c2cccc3c2sc2c(N(c4ccc(-c5ccc6ccccc6c5)cc4)c4cccc5oc6ccccc6c45)cccc23)cc1 ; c1ccc(-c2cccc3c2sc2c(N(c4ccc(-c5cc6ccccc6c6ccccc56)cc4)c4cccc5oc6ccccc6c45)cccc23)cc1]

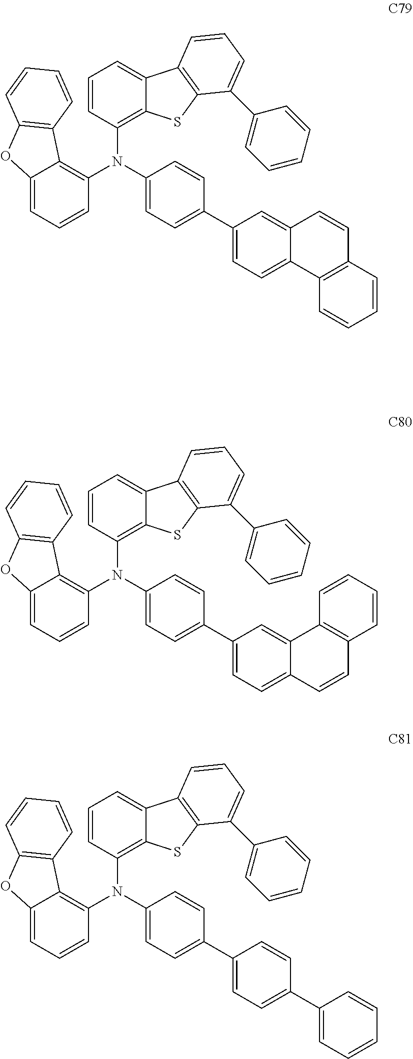 [SMILES: c1ccc(-c2cccc3c2sc2c(N(c4ccc(-c5ccc6c(ccc7ccccc76)c5)cc4)c4cccc5oc6ccccc6c45)cccc23)cc1 ; c1ccc(-c2cccc3c2sc2c(N(c4ccc(-c5ccc6ccc7ccccc7c6c5)cc4)c4cccc5oc6ccccc6c45)cccc23)cc1 ; c1ccc(-c2ccc(-c3ccc(N(c4cccc5c4sc4c(-c6ccccc6)cccc45)c4cccc5oc6ccccc6c45)cc3)cc2)cc1]

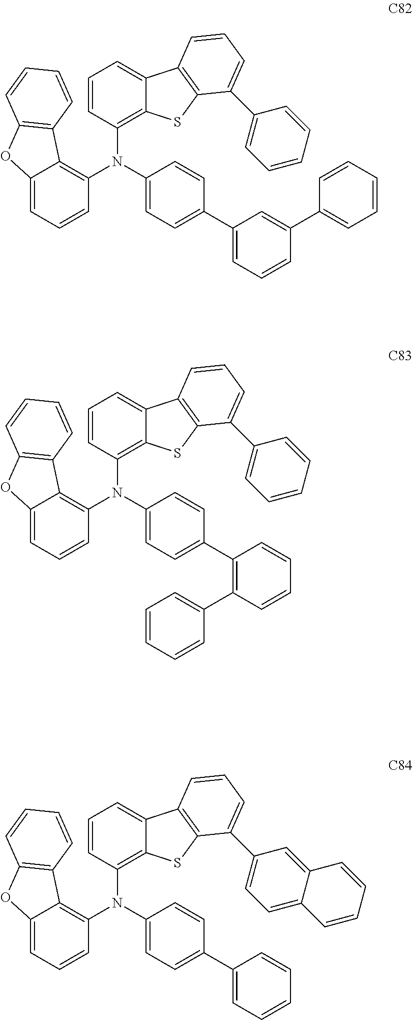 [SMILES: c1ccc(-c2cccc(-c3ccc(N(c4cccc5c4sc4c(-c6ccccc6)cccc45)c4cccc5oc6ccccc6c45)cc3)c2)cc1 ; c1ccc(-c2ccccc2-c2ccc(N(c3cccc4c3sc3c(-c5ccccc5)cccc34)c3cccc4oc5ccccc5c34)cc2)cc1 ; c1ccc(-c2ccc(N(c3cccc4c3sc3c(-c5ccc6ccccc6c5)cccc34)c3cccc4oc5ccccc5c34)cc2)cc1]

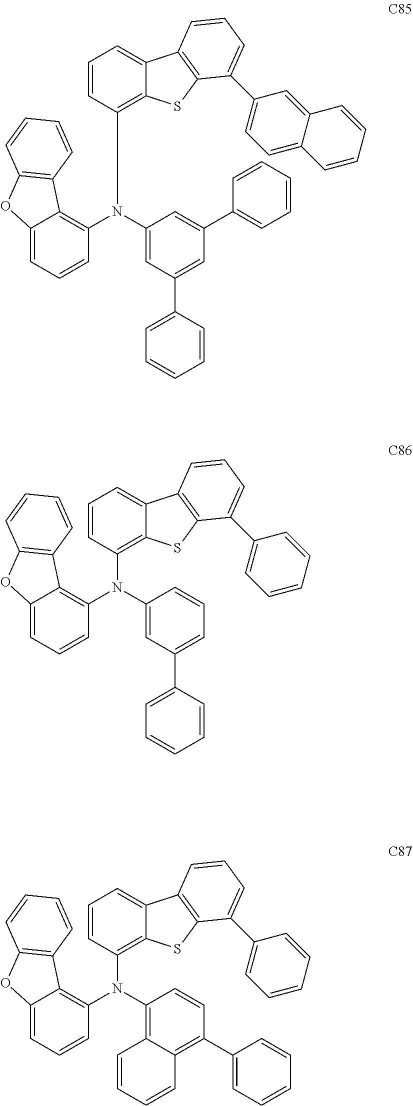 [SMILES: c1ccc(-c2cc(-c3ccccc3)cc(N(c3cccc4c3sc3c(-c5ccc6ccccc6c5)cccc34)c3cccc4oc5ccccc5c34)c2)cc1 ; c1ccc(-c2cccc(N(c3cccc4c3sc3c(-c5ccccc5)cccc34)c3cccc4oc5ccccc5c34)c2)cc1 ; c1ccc(-c2ccc(N(c3cccc4c3sc3c(-c5ccccc5)cccc34)c3cccc4oc5ccccc5c34)c3ccccc23)cc1]

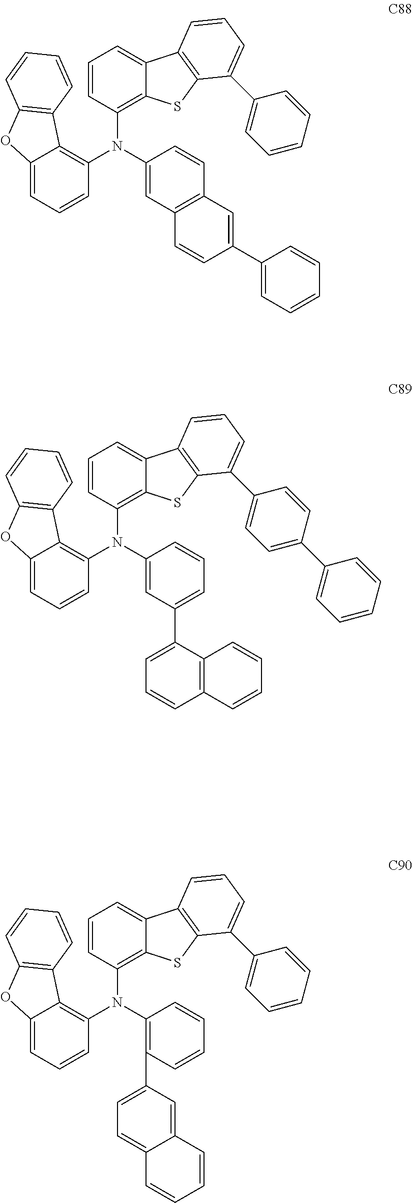 [SMILES: c1ccc(-c2ccc3cc(N(c4cccc5c4sc4c(-c6ccccc6)cccc45)c4cccc5oc6ccccc6c45)ccc3c2)cc1 ; c1ccc(-c2ccc(-c3cccc4c3sc3c(N(c5cccc(-c6cccc7ccccc67)c5)c5cccc6oc7ccccc7c56)cccc34)cc2)cc1 ; c1ccc(-c2cccc3c2sc2c(N(c4ccccc4-c4ccc5ccccc5c4)c4cccc5oc6ccccc6c45)cccc23)cc1]

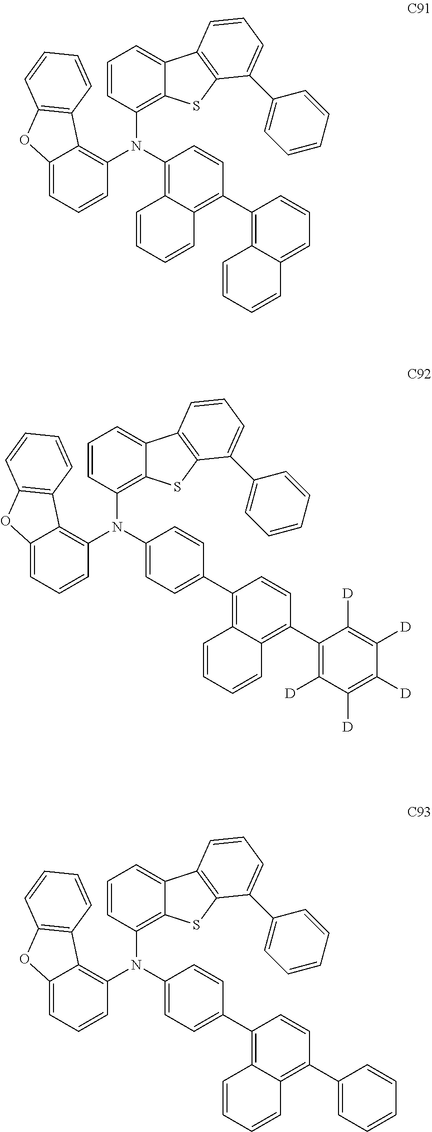 [SMILES: c1ccc(-c2cccc3c2sc2c(N(c4ccc(-c5cccc6ccccc56)c5ccccc45)c4cccc5oc6ccccc6c45)cccc23)cc1 ; [2H]c1c([2H])c([2H])c(-c2ccc(-c3ccc(N(c4cccc5c4sc4c(-c6ccccc6)cccc45)c4cccc5oc6ccccc6c45)cc3)c3ccccc23)c([2H])c1[2H] ; c1ccc(-c2ccc(-c3ccc(N(c4cccc5c4sc4c(-c6ccccc6)cccc45)c4cccc5oc6ccccc6c45)cc3)c3ccccc23)cc1]

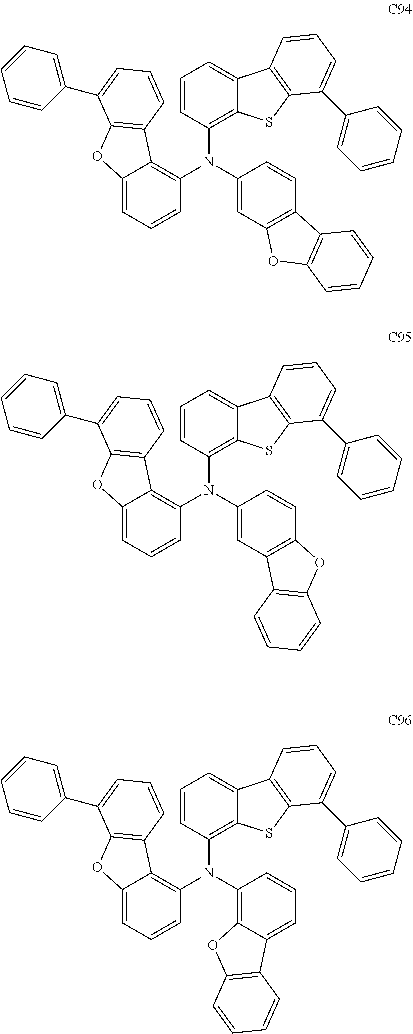 [SMILES: c1ccc(-c2cccc3c2oc2cccc(N(c4ccc5c(c4)oc4ccccc45)c4cccc5c4sc4c(-c6ccccc6)cccc45)c23)cc1 ; c1ccc(-c2cccc3c2oc2cccc(N(c4ccc5oc6ccccc6c5c4)c4cccc5c4sc4c(-c6ccccc6)cccc45)c23)cc1 ; c1ccc(-c2cccc3c2oc2cccc(N(c4cccc5c4oc4ccccc45)c4cccc5c4sc4c(-c6ccccc6)cccc45)c23)cc1]

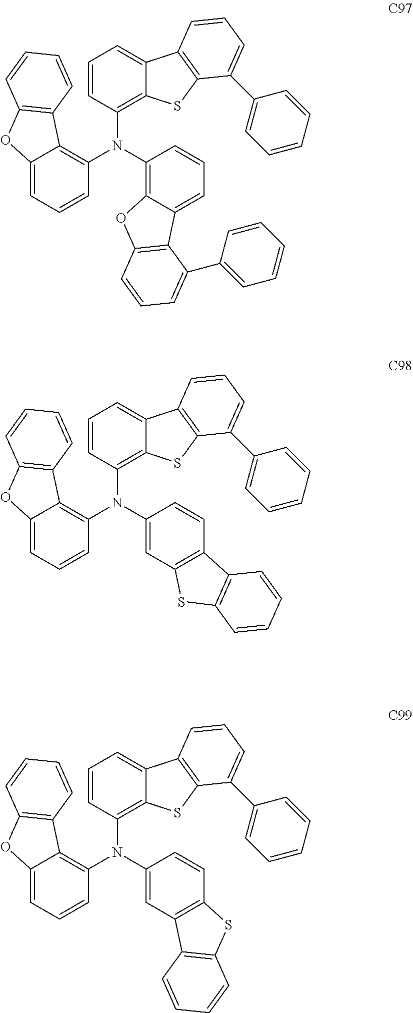 [SMILES: c1ccc(-c2cccc3c2sc2c(N(c4cccc5c4oc4cccc(-c6ccccc6)c45)c4cccc5oc6ccccc6c45)cccc23)cc1 ; c1ccc(-c2cccc3c2sc2c(N(c4ccc5c(c4)sc4ccccc45)c4cccc5oc6ccccc6c45)cccc23)cc1 ; c1ccc(-c2cccc3c2sc2c(N(c4ccc5sc6ccccc6c5c4)c4cccc5oc6ccccc6c45)cccc23)cc1]

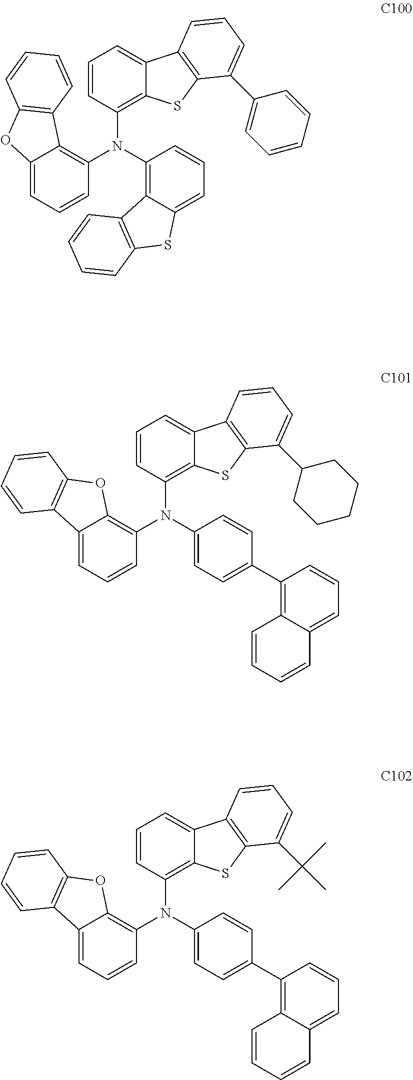 [SMILES: c1ccc(-c2cccc3c2sc2c(N(c4cccc5oc6ccccc6c45)c4cccc5sc6ccccc6c45)cccc23)cc1 ; c1ccc2c(-c3ccc(N(c4cccc5c4oc4ccccc45)c4cccc5c4sc4c(C6CCCCC6)cccc45)cc3)cccc2c1 ; CC(C)(C)c1cccc2c1sc1c(N(c3ccc(-c4cccc5ccccc45)cc3)c3cccc4c3oc3ccccc34)cccc12]

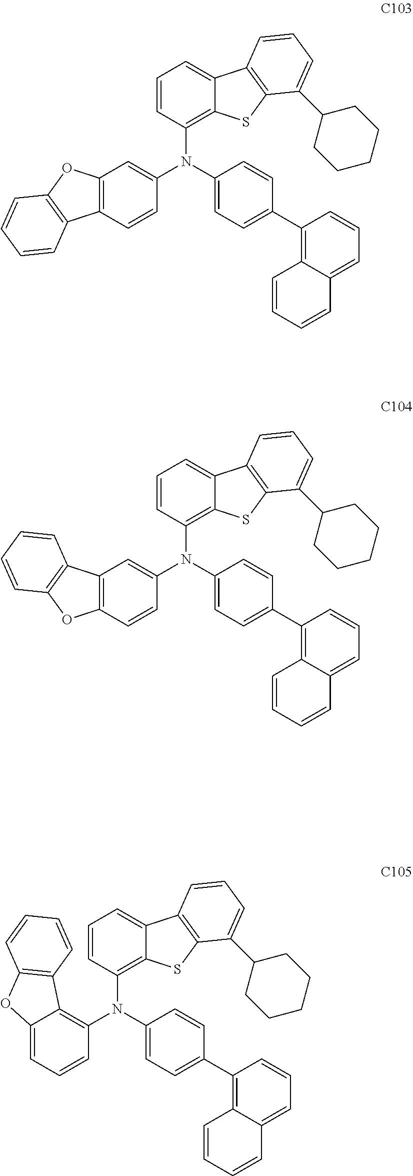 [SMILES: c1ccc2c(-c3ccc(N(c4ccc5c(c4)oc4ccccc45)c4cccc5c4sc4c(C6CCCCC6)cccc45)cc3)cccc2c1 ; c1ccc2c(-c3ccc(N(c4ccc5oc6ccccc6c5c4)c4cccc5c4sc4c(C6CCCCC6)cccc45)cc3)cccc2c1 ; c1ccc2c(-c3ccc(N(c4cccc5c4sc4c(C6CCCCC6)cccc45)c4cccc5oc6ccccc6c45)cc3)cccc2c1]

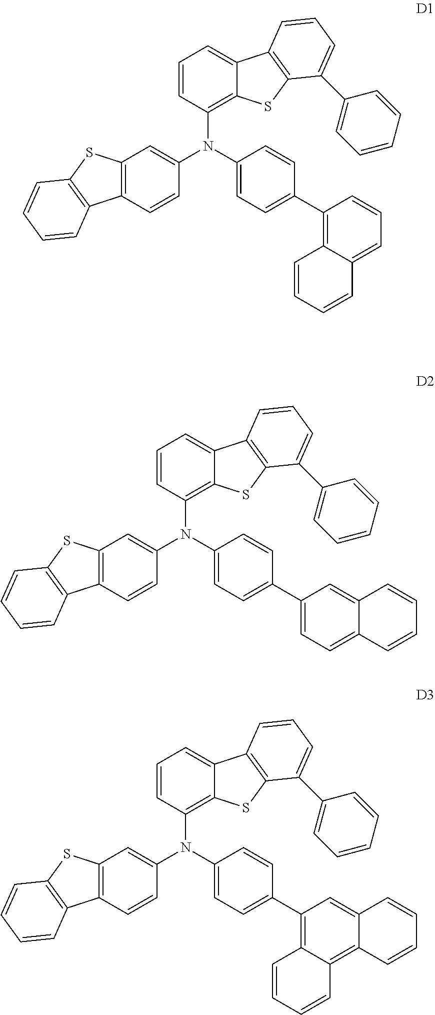 [SMILES: c1ccc(-c2cccc3c2sc2c(N(c4ccc(-c5cccc6ccccc56)cc4)c4ccc5c(c4)sc4ccccc45)cccc23)cc1 ; c1ccc(-c2cccc3c2sc2c(N(c4ccc(-c5ccc6ccccc6c5)cc4)c4ccc5c(c4)sc4ccccc45)cccc23)cc1 ; c1ccc(-c2cccc3c2sc2c(N(c4ccc(-c5cc6ccccc6c6ccccc56)cc4)c4ccc5c(c4)sc4ccccc45)cccc23)cc1]

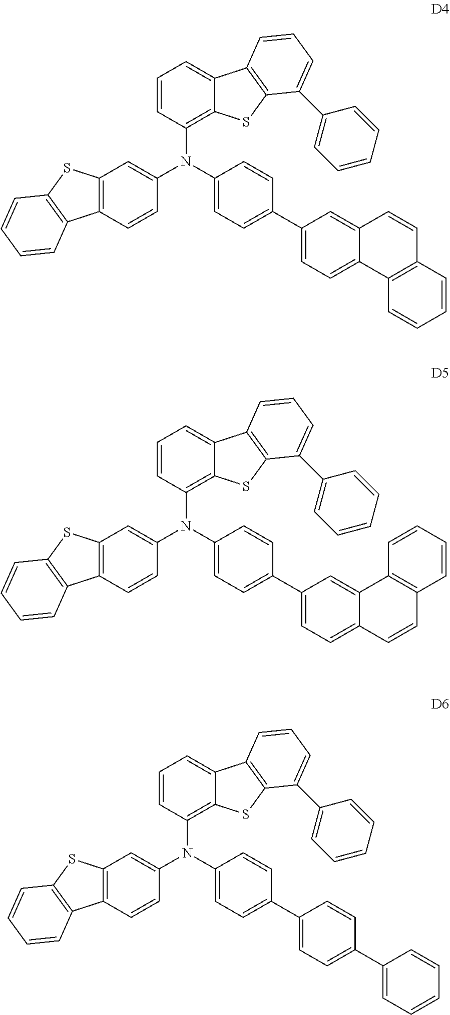 [SMILES: c1ccc(-c2cccc3c2sc2c(N(c4ccc(-c5ccc6c(ccc7ccccc76)c5)cc4)c4ccc5c(c4)sc4ccccc45)cccc23)cc1 ; c1ccc(-c2cccc3c2sc2c(N(c4ccc(-c5ccc6ccc7ccccc7c6c5)cc4)c4ccc5c(c4)sc4ccccc45)cccc23)cc1 ; c1ccc(-c2ccc(-c3ccc(N(c4ccc5c(c4)sc4ccccc45)c4cccc5c4sc4c(-c6ccccc6)cccc45)cc3)cc2)cc1]

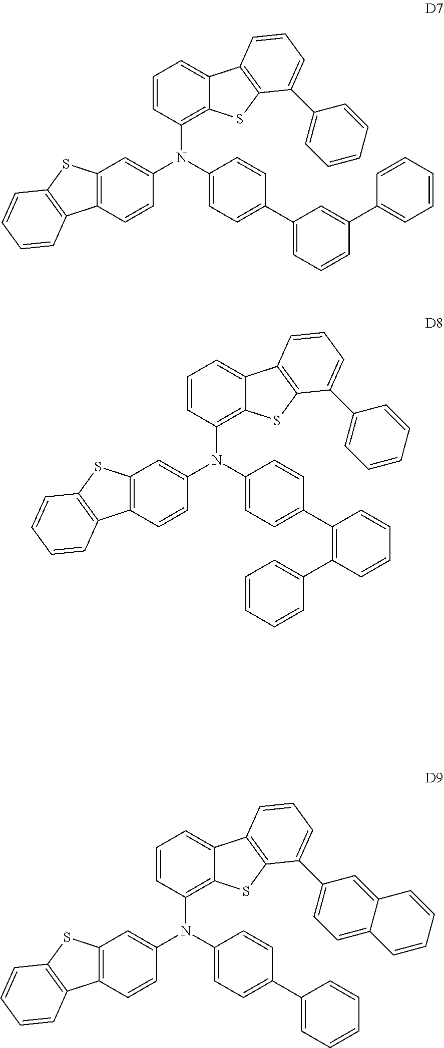 [SMILES: c1ccc(-c2cccc(-c3ccc(N(c4ccc5c(c4)sc4ccccc45)c4cccc5c4sc4c(-c6ccccc6)cccc45)cc3)c2)cc1 ; c1ccc(-c2ccccc2-c2ccc(N(c3ccc4c(c3)sc3ccccc34)c3cccc4c3sc3c(-c5ccccc5)cccc34)cc2)cc1 ; c1ccc(-c2ccc(N(c3ccc4c(c3)sc3ccccc34)c3cccc4c3sc3c(-c5ccc6ccccc6c5)cccc34)cc2)cc1]

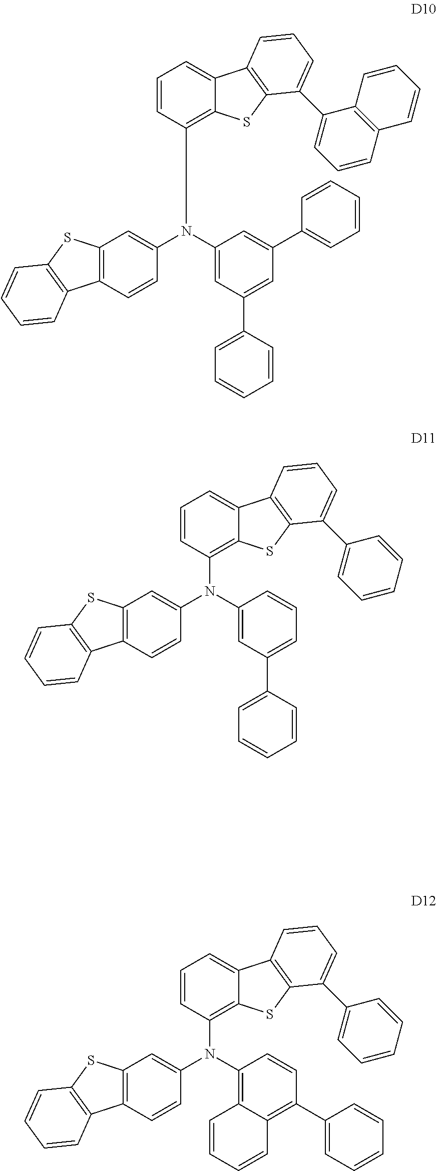 [SMILES: c1ccc(-c2cc(-c3ccccc3)cc(N(c3ccc4c(c3)sc3ccccc34)c3cccc4c3sc3c(-c5cccc6ccccc56)cccc34)c2)cc1 ; c1ccc(-c2cccc(N(c3ccc4c(c3)sc3ccccc34)c3cccc4c3sc3c(-c5ccccc5)cccc34)c2)cc1 ; c1ccc(-c2ccc(N(c3ccc4c(c3)sc3ccccc34)c3cccc4c3sc3c(-c5ccccc5)cccc34)c3ccccc23)cc1]

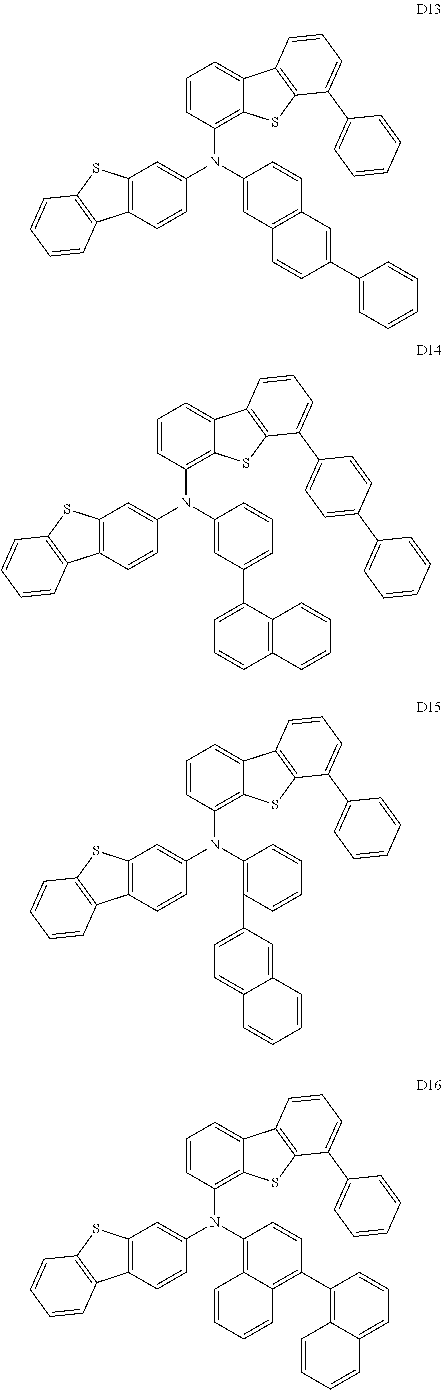 [SMILES: c1ccc(-c2ccc3cc(N(c4ccc5c(c4)sc4ccccc45)c4cccc5c4sc4c(-c6ccccc6)cccc45)ccc3c2)cc1 ; c1ccc(-c2ccc(-c3cccc4c3sc3c(N(c5cccc(-c6cccc7ccccc67)c5)c5ccc6c(c5)sc5ccccc56)cccc34)cc2)cc1 ; c1ccc(-c2cccc3c2sc2c(N(c4ccc5c(c4)sc4ccccc45)c4ccccc4-c4ccc5ccccc5c4)cccc23)cc1 ; c1ccc(-c2cccc3c2sc2c(N(c4ccc5c(c4)sc4ccccc45)c4ccc(-c5cccc6ccccc56)c5ccccc45)cccc23)cc1]

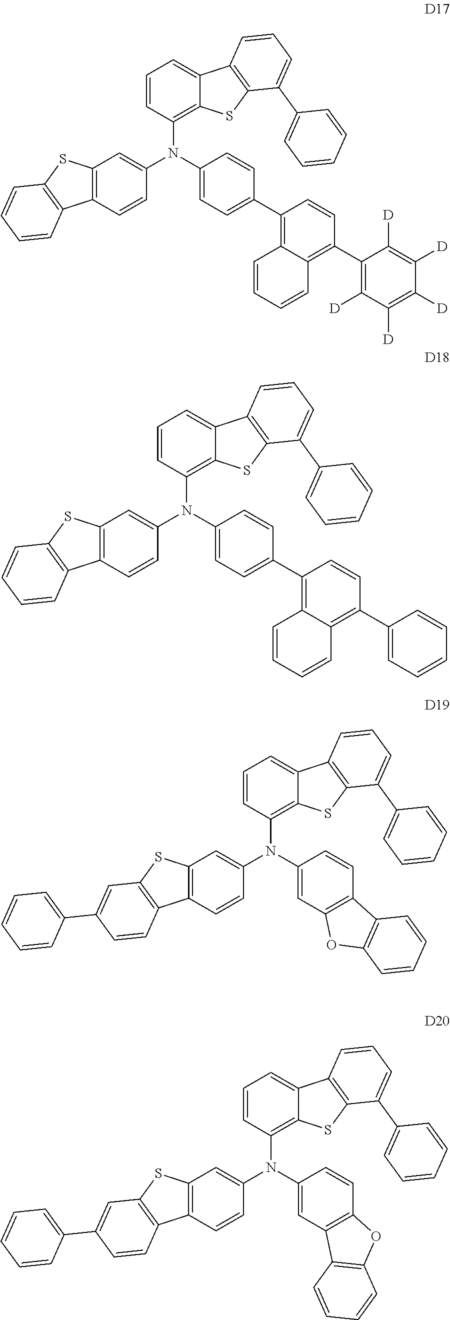 [SMILES: [2H]c1c([2H])c([2H])c(-c2ccc(-c3ccc(N(c4ccc5c(c4)sc4ccccc45)c4cccc5c4sc4c(-c6ccccc6)cccc45)cc3)c3ccccc23)c([2H])c1[2H] ; c1ccc(-c2ccc(-c3ccc(N(c4ccc5c(c4)sc4ccccc45)c4cccc5c4sc4c(-c6ccccc6)cccc45)cc3)c3ccccc23)cc1 ; c1ccc(-c2ccc3c(c2)sc2cc(N(c4ccc5c(c4)oc4ccccc45)c4cccc5c4sc4c(-c6ccccc6)cccc45)ccc23)cc1 ; c1ccc(-c2ccc3c(c2)sc2cc(N(c4ccc5oc6ccccc6c5c4)c4cccc5c4sc4c(-c6ccccc6)cccc45)ccc23)cc1]

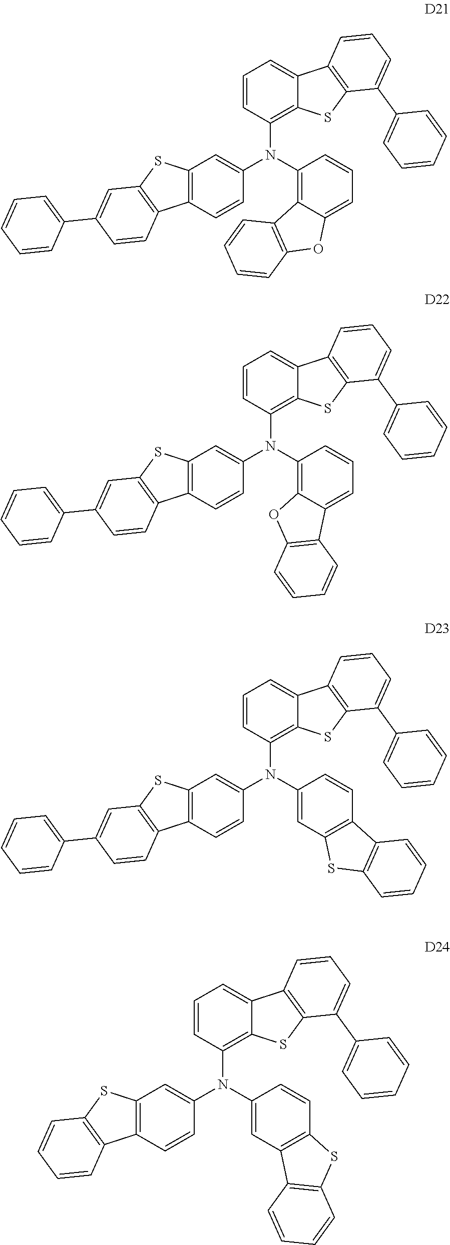 [SMILES: c1ccc(-c2ccc3c(c2)sc2cc(N(c4cccc5c4sc4c(-c6ccccc6)cccc45)c4cccc5oc6ccccc6c45)ccc23)cc1 ; c1ccc(-c2ccc3c(c2)sc2cc(N(c4cccc5c4oc4ccccc45)c4cccc5c4sc4c(-c6ccccc6)cccc45)ccc23)cc1 ; c1ccc(-c2ccc3c(c2)sc2cc(N(c4ccc5c(c4)sc4ccccc45)c4cccc5c4sc4c(-c6ccccc6)cccc45)ccc23)cc1 ; c1ccc(-c2cccc3c2sc2c(N(c4ccc5c(c4)sc4ccccc45)c4ccc5sc6ccccc6c5c4)cccc23)cc1]

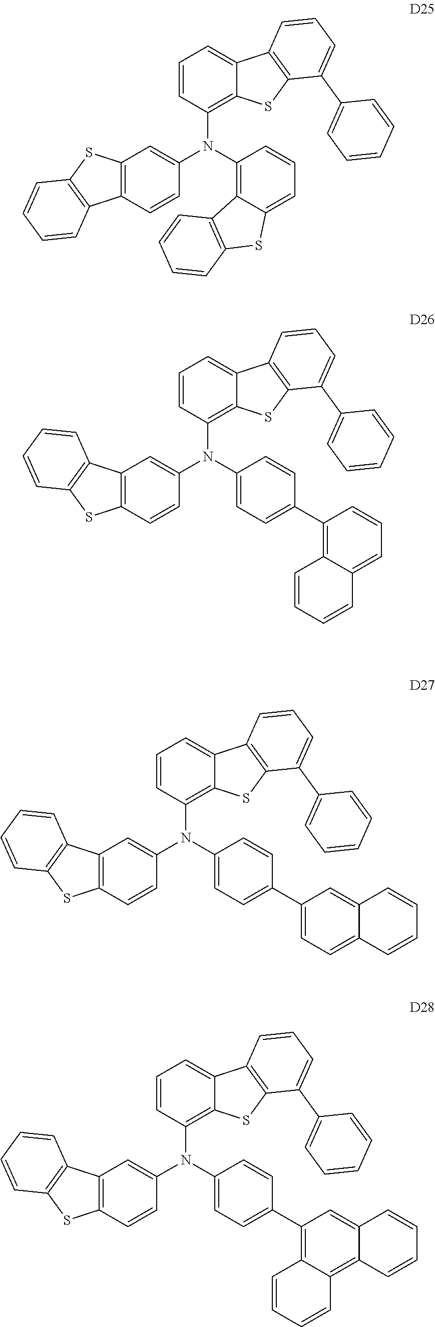 [SMILES: c1ccc(-c2cccc3c2sc2c(N(c4ccc5c(c4)sc4ccccc45)c4cccc5sc6ccccc6c45)cccc23)cc1 ; c1ccc(-c2cccc3c2sc2c(N(c4ccc(-c5cccc6ccccc56)cc4)c4ccc5sc6ccccc6c5c4)cccc23)cc1 ; c1ccc(-c2cccc3c2sc2c(N(c4ccc(-c5ccc6ccccc6c5)cc4)c4ccc5sc6ccccc6c5c4)cccc23)cc1 ; c1ccc(-c2cccc3c2sc2c(N(c4ccc(-c5cc6ccccc6c6ccccc56)cc4)c4ccc5sc6ccccc6c5c4)cccc23)cc1]

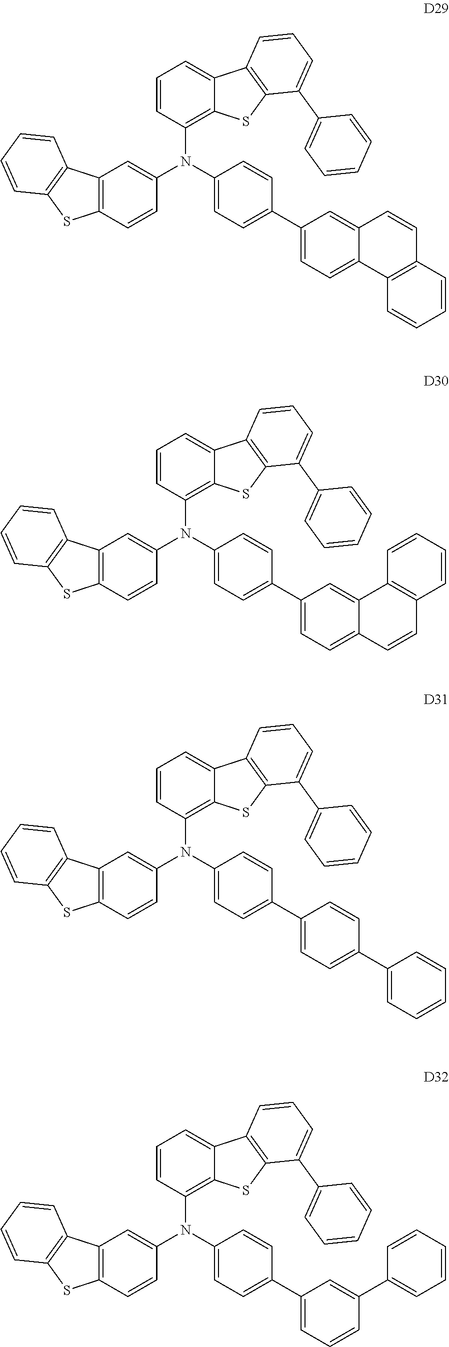 [SMILES: c1ccc(-c2cccc3c2sc2c(N(c4ccc(-c5ccc6c(ccc7ccccc76)c5)cc4)c4ccc5sc6ccccc6c5c4)cccc23)cc1 ; c1ccc(-c2cccc3c2sc2c(N(c4ccc(-c5ccc6ccc7ccccc7c6c5)cc4)c4ccc5sc6ccccc6c5c4)cccc23)cc1 ; c1ccc(-c2ccc(-c3ccc(N(c4ccc5sc6ccccc6c5c4)c4cccc5c4sc4c(-c6ccccc6)cccc45)cc3)cc2)cc1 ; c1ccc(-c2cccc(-c3ccc(N(c4ccc5sc6ccccc6c5c4)c4cccc5c4sc4c(-c6ccccc6)cccc45)cc3)c2)cc1]

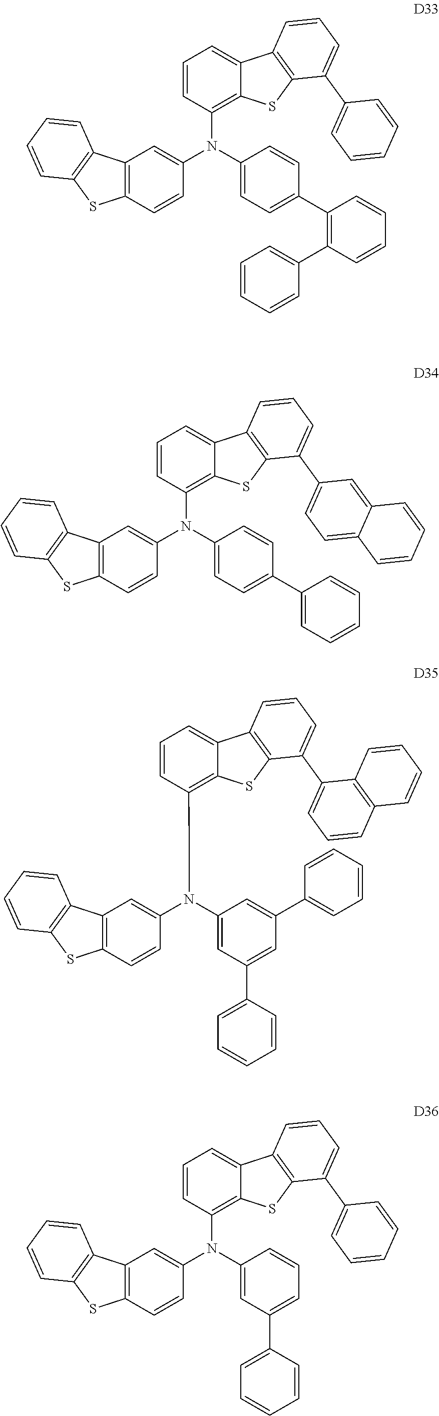 [SMILES: c1ccc(-c2ccccc2-c2ccc(N(c3ccc4sc5ccccc5c4c3)c3cccc4c3sc3c(-c5ccccc5)cccc34)cc2)cc1 ; c1ccc(-c2ccc(N(c3ccc4sc5ccccc5c4c3)c3cccc4c3sc3c(-c5ccc6ccccc6c5)cccc34)cc2)cc1 ; c1ccc(-c2cc(-c3ccccc3)cc(N(c3ccc4sc5ccccc5c4c3)c3cccc4c3sc3c(-c5cccc6ccccc56)cccc34)c2)cc1 ; c1ccc(-c2cccc(N(c3ccc4sc5ccccc5c4c3)c3cccc4c3sc3c(-c5ccccc5)cccc34)c2)cc1]

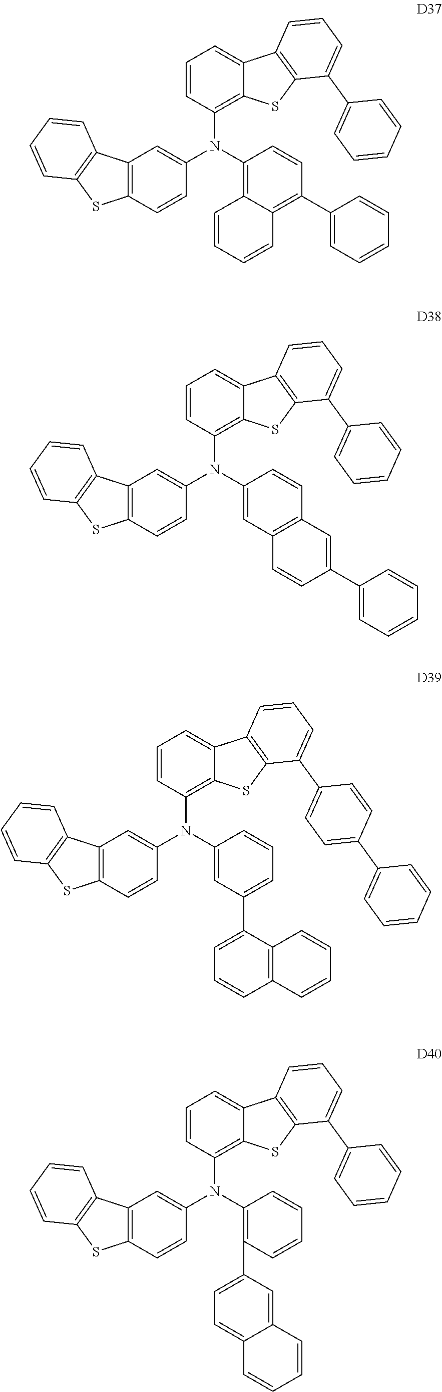 [SMILES: c1ccc(-c2ccc(N(c3ccc4sc5ccccc5c4c3)c3cccc4c3sc3c(-c5ccccc5)cccc34)c3ccccc23)cc1 ; c1ccc(-c2ccc3cc(N(c4ccc5sc6ccccc6c5c4)c4cccc5c4sc4c(-c6ccccc6)cccc45)ccc3c2)cc1 ; c1ccc(-c2ccc(-c3cccc4c3sc3c(N(c5cccc(-c6cccc7ccccc67)c5)c5ccc6sc7ccccc7c6c5)cccc34)cc2)cc1 ; c1ccc(-c2cccc3c2sc2c(N(c4ccc5sc6ccccc6c5c4)c4ccccc4-c4ccc5ccccc5c4)cccc23)cc1]

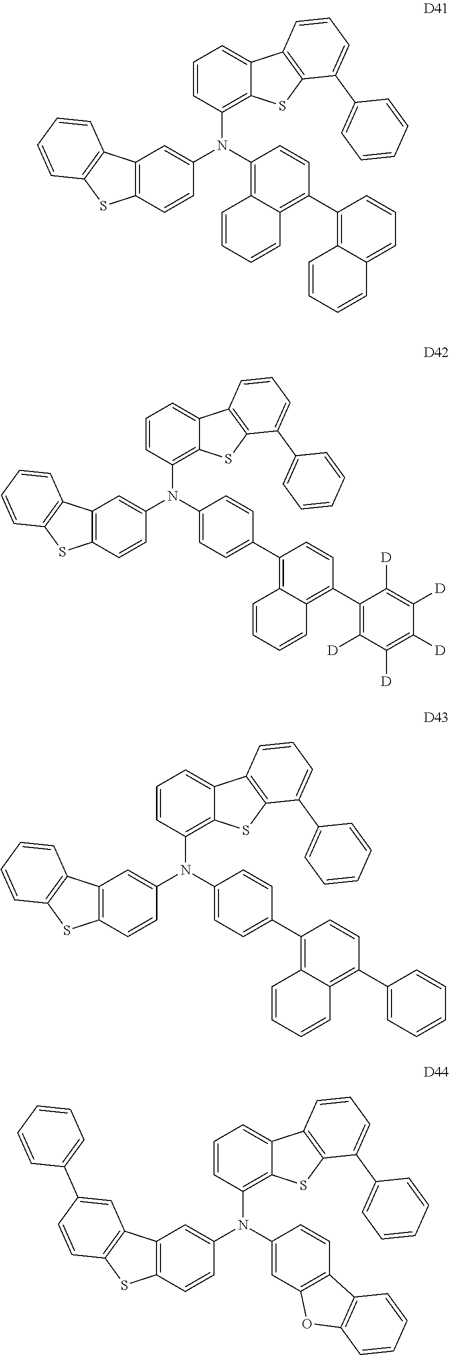 [SMILES: c1ccc(-c2cccc3c2sc2c(N(c4ccc5sc6ccccc6c5c4)c4ccc(-c5cccc6ccccc56)c5ccccc45)cccc23)cc1 ; [2H]c1c([2H])c([2H])c(-c2ccc(-c3ccc(N(c4ccc5sc6ccccc6c5c4)c4cccc5c4sc4c(-c6ccccc6)cccc45)cc3)c3ccccc23)c([2H])c1[2H] ; c1ccc(-c2ccc(-c3ccc(N(c4ccc5sc6ccccc6c5c4)c4cccc5c4sc4c(-c6ccccc6)cccc45)cc3)c3ccccc23)cc1 ; c1ccc(-c2ccc3sc4ccc(N(c5ccc6c(c5)oc5ccccc56)c5cccc6c5sc5c(-c7ccccc7)cccc56)cc4c3c2)cc1]

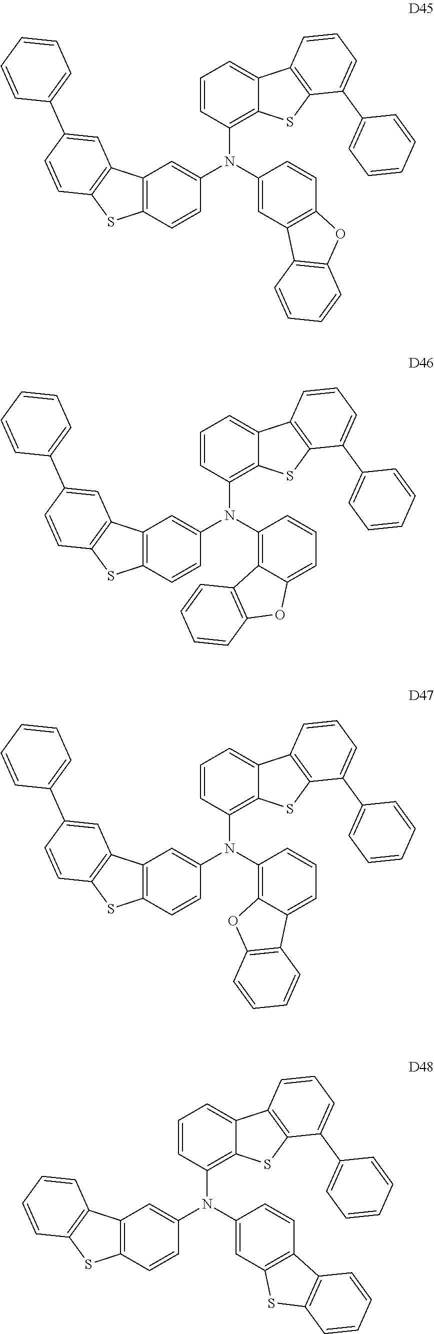 [SMILES: c1ccc(-c2ccc3sc4ccc(N(c5ccc6oc7ccccc7c6c5)c5cccc6c5sc5c(-c7ccccc7)cccc56)cc4c3c2)cc1 ; c1ccc(-c2ccc3sc4ccc(N(c5cccc6c5sc5c(-c7ccccc7)cccc56)c5cccc6oc7ccccc7c56)cc4c3c2)cc1 ; c1ccc(-c2ccc3sc4ccc(N(c5cccc6c5oc5ccccc56)c5cccc6c5sc5c(-c7ccccc7)cccc56)cc4c3c2)cc1 ; c1ccc(-c2cccc3c2sc2c(N(c4ccc5c(c4)sc4ccccc45)c4ccc5sc6ccccc6c5c4)cccc23)cc1]

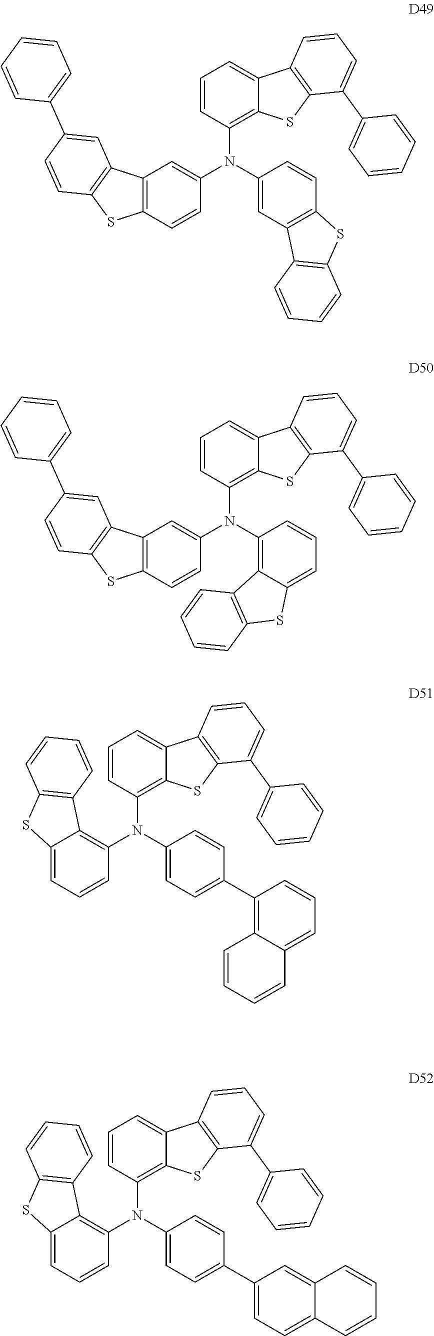 [SMILES: c1ccc(-c2ccc3sc4ccc(N(c5ccc6sc7ccccc7c6c5)c5cccc6c5sc5c(-c7ccccc7)cccc56)cc4c3c2)cc1 ; c1ccc(-c2ccc3sc4ccc(N(c5cccc6c5sc5c(-c7ccccc7)cccc56)c5cccc6sc7ccccc7c56)cc4c3c2)cc1 ; c1ccc(-c2cccc3c2sc2c(N(c4ccc(-c5cccc6ccccc56)cc4)c4cccc5sc6ccccc6c45)cccc23)cc1 ; c1ccc(-c2cccc3c2sc2c(N(c4ccc(-c5ccc6ccccc6c5)cc4)c4cccc5sc6ccccc6c45)cccc23)cc1]

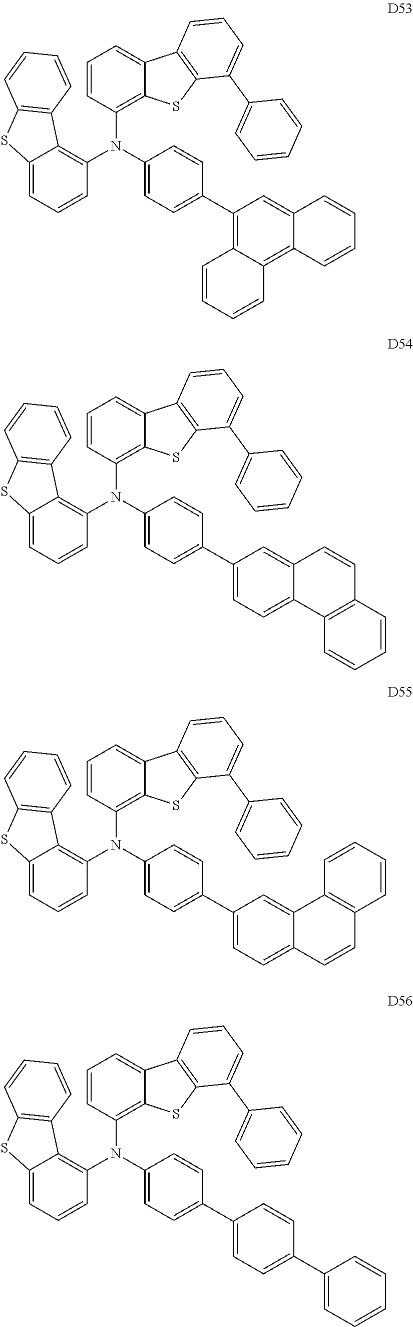 [SMILES: c1ccc(-c2cccc3c2sc2c(N(c4ccc(-c5cc6ccccc6c6ccccc56)cc4)c4cccc5sc6ccccc6c45)cccc23)cc1 ; c1ccc(-c2cccc3c2sc2c(N(c4ccc(-c5ccc6c(ccc7ccccc76)c5)cc4)c4cccc5sc6ccccc6c45)cccc23)cc1 ; c1ccc(-c2cccc3c2sc2c(N(c4ccc(-c5ccc6ccc7ccccc7c6c5)cc4)c4cccc5sc6ccccc6c45)cccc23)cc1 ; c1ccc(-c2ccc(-c3ccc(N(c4cccc5c4sc4c(-c6ccccc6)cccc45)c4cccc5sc6ccccc6c45)cc3)cc2)cc1]

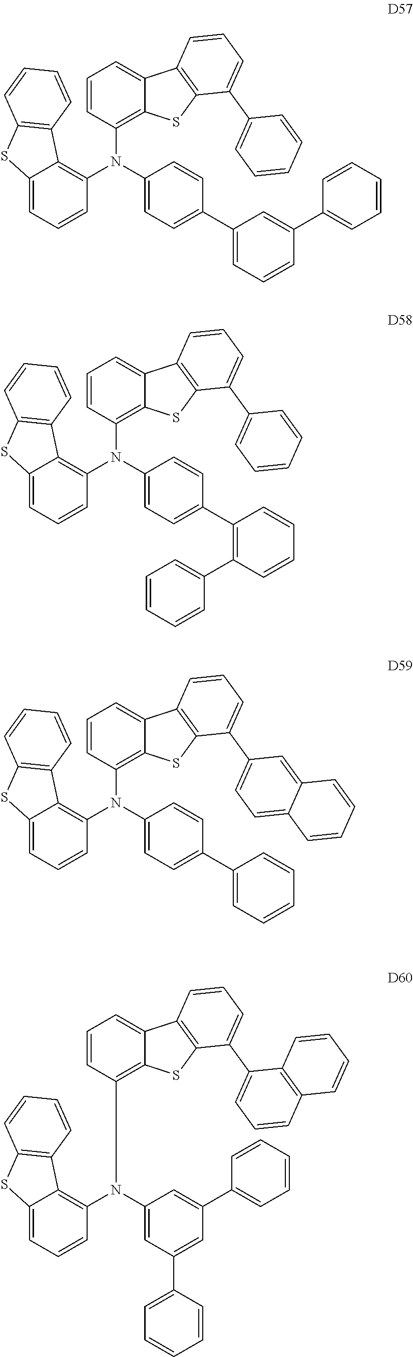 [SMILES: c1ccc(-c2cccc(-c3ccc(N(c4cccc5c4sc4c(-c6ccccc6)cccc45)c4cccc5sc6ccccc6c45)cc3)c2)cc1 ; c1ccc(-c2ccccc2-c2ccc(N(c3cccc4c3sc3c(-c5ccccc5)cccc34)c3cccc4sc5ccccc5c34)cc2)cc1 ; c1ccc(-c2ccc(N(c3cccc4c3sc3c(-c5ccc6ccccc6c5)cccc34)c3cccc4sc5ccccc5c34)cc2)cc1 ; c1ccc(-c2cc(-c3ccccc3)cc(N(c3cccc4c3sc3c(-c5cccc6ccccc56)cccc34)c3cccc4sc5ccccc5c34)c2)cc1]

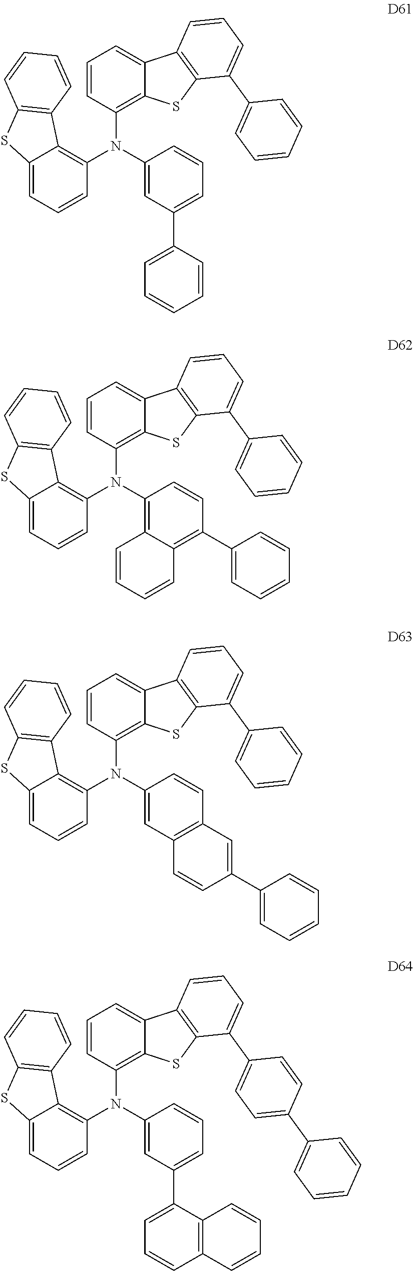 [SMILES: c1ccc(-c2cccc(N(c3cccc4c3sc3c(-c5ccccc5)cccc34)c3cccc4sc5ccccc5c34)c2)cc1 ; c1ccc(-c2ccc(N(c3cccc4c3sc3c(-c5ccccc5)cccc34)c3cccc4sc5ccccc5c34)c3ccccc23)cc1 ; c1ccc(-c2ccc3cc(N(c4cccc5c4sc4c(-c6ccccc6)cccc45)c4cccc5sc6ccccc6c45)ccc3c2)cc1 ; c1ccc(-c2ccc(-c3cccc4c3sc3c(N(c5cccc(-c6cccc7ccccc67)c5)c5cccc6sc7ccccc7c56)cccc34)cc2)cc1]

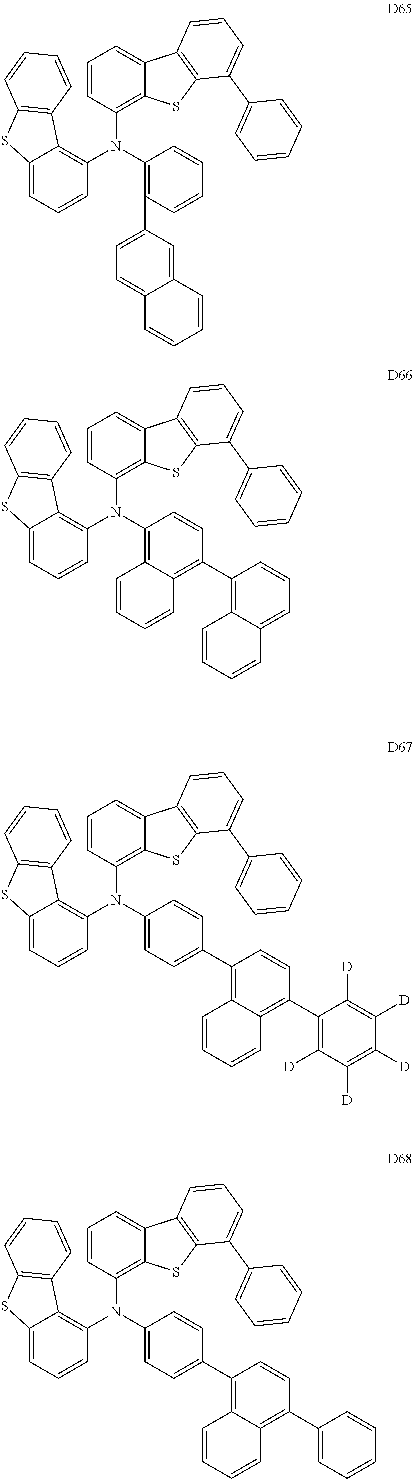

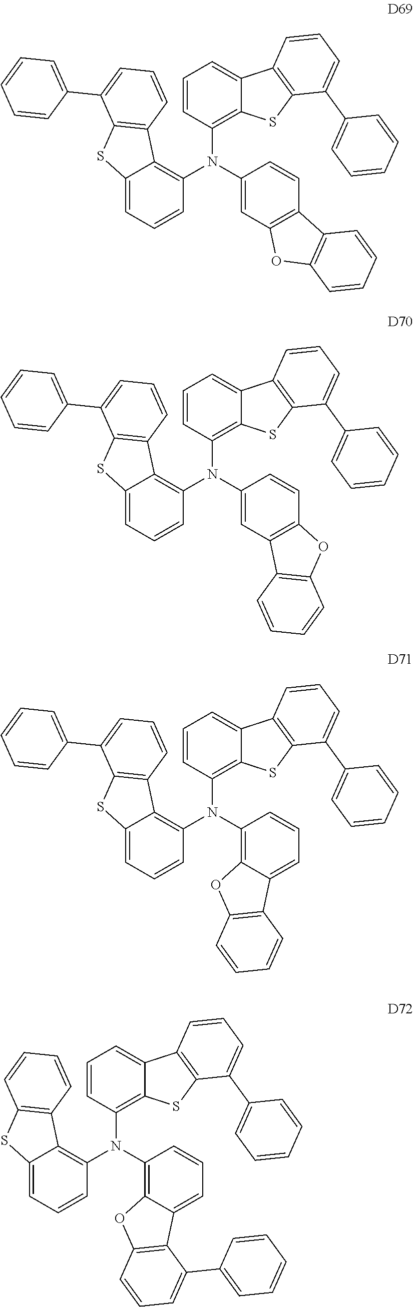 [SMILES: c1ccc(-c2cccc3c2sc2c(N(c4ccc5c(c4)oc4ccccc45)c4cccc5sc6c(-c7ccccc7)cccc6c45)cccc23)cc1 ; c1ccc(-c2cccc3c2sc2c(N(c4ccc5oc6ccccc6c5c4)c4cccc5sc6c(-c7ccccc7)cccc6c45)cccc23)cc1 ; c1ccc(-c2cccc3c2sc2c(N(c4cccc5c4oc4ccccc45)c4cccc5sc6c(-c7ccccc7)cccc6c45)cccc23)cc1 ; c1ccc(-c2cccc3c2sc2c(N(c4cccc5c4oc4cccc(-c6ccccc6)c45)c4cccc5sc6ccccc6c45)cccc23)cc1]

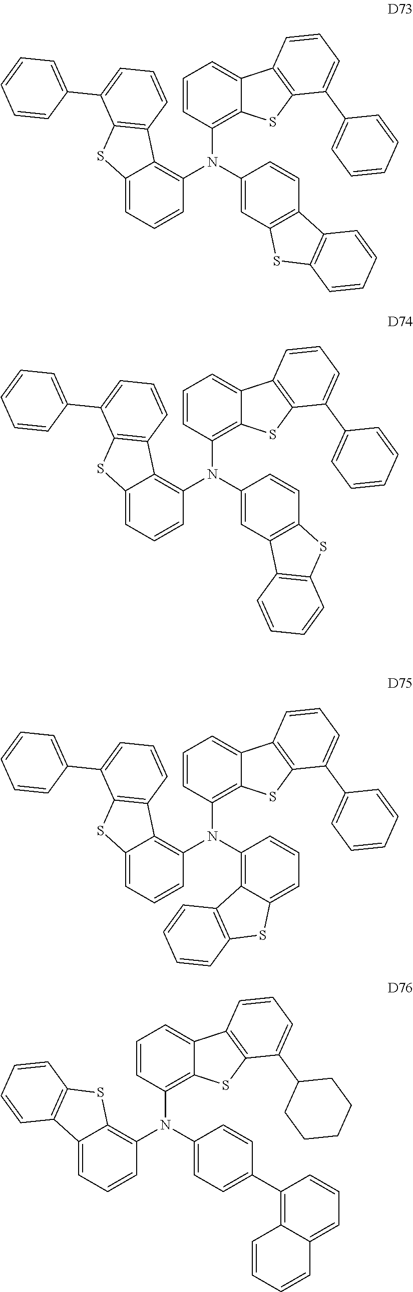

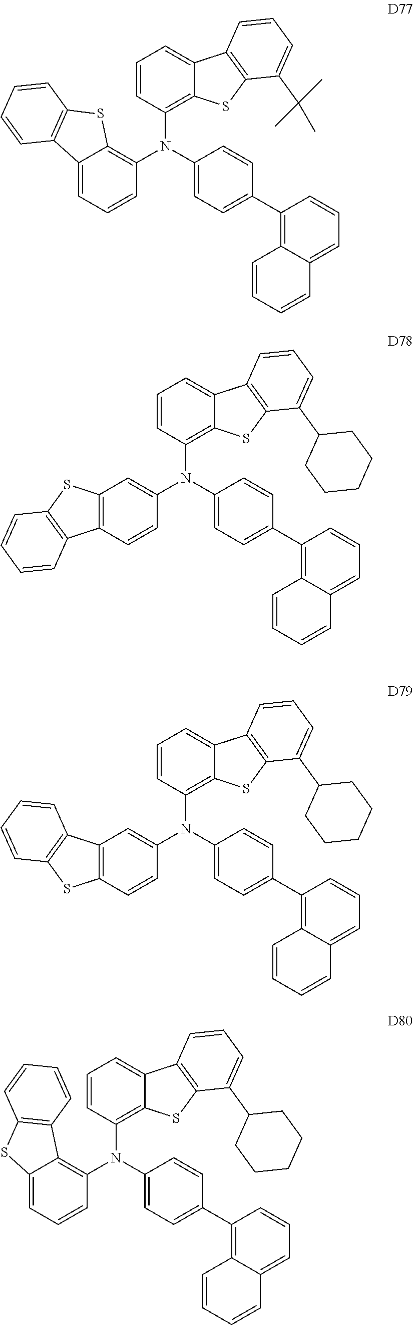

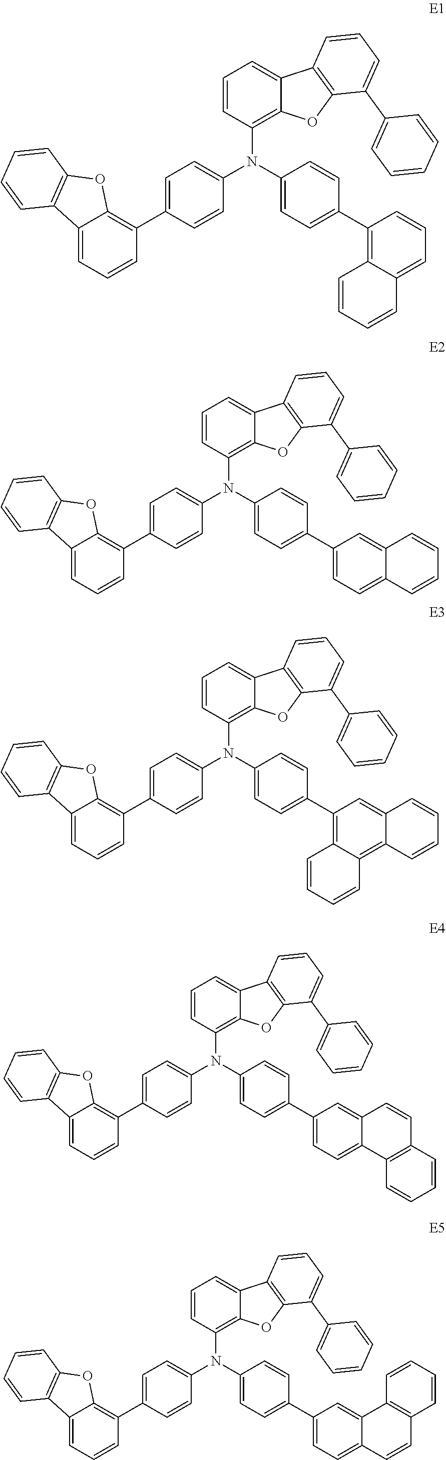 [SMILES: c1ccc(-c2cccc3c2oc2c(N(c4ccc(-c5cccc6ccccc56)cc4)c4ccc(-c5cccc6c5oc5ccccc56)cc4)cccc23)cc1 ; c1ccc(-c2cccc3c2oc2c(N(c4ccc(-c5ccc6ccccc6c5)cc4)c4ccc(-c5cccc6c5oc5ccccc56)cc4)cccc23)cc1 ; c1ccc(-c2cccc3c2oc2c(N(c4ccc(-c5cc6ccccc6c6ccccc56)cc4)c4ccc(-c5cccc6c5oc5ccccc56)cc4)cccc23)cc1 ; c1ccc(-c2cccc3c2oc2c(N(c4ccc(-c5ccc6c(ccc7ccccc76)c5)cc4)c4ccc(-c5cccc6c5oc5ccccc56)cc4)cccc23)cc1 ; c1ccc(-c2cccc3c2oc2c(N(c4ccc(-c5ccc6ccc7ccccc7c6c5)cc4)c4ccc(-c5cccc6c5oc5ccccc56)cc4)cccc23)cc1]

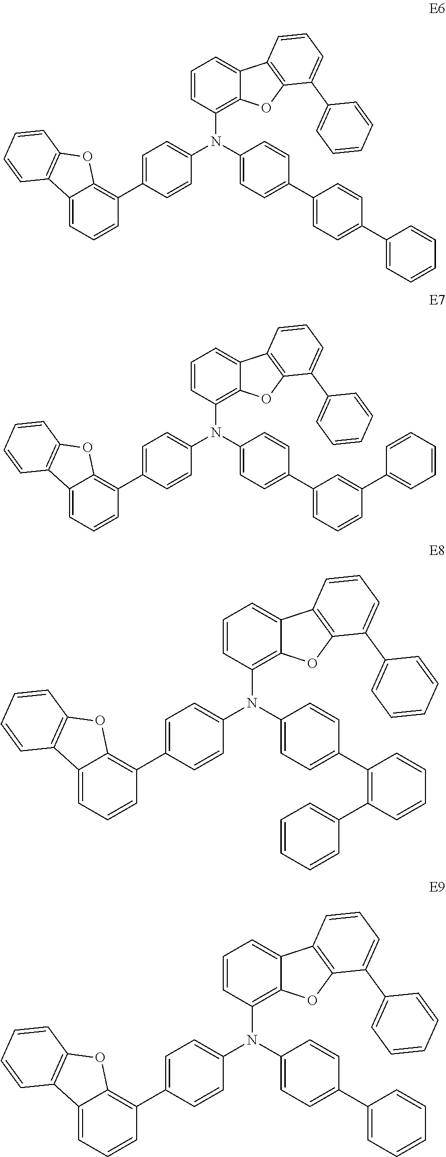 [SMILES: c1ccc(-c2ccc(-c3ccc(N(c4ccc(-c5cccc6c5oc5ccccc56)cc4)c4cccc5c4oc4c(-c6ccccc6)cccc45)cc3)cc2)cc1 ; c1ccc(-c2cccc(-c3ccc(N(c4ccc(-c5cccc6c5oc5ccccc56)cc4)c4cccc5c4oc4c(-c6ccccc6)cccc45)cc3)c2)cc1 ; c1ccc(-c2ccccc2-c2ccc(N(c3ccc(-c4cccc5c4oc4ccccc45)cc3)c3cccc4c3oc3c(-c5ccccc5)cccc34)cc2)cc1 ; c1ccc(-c2ccc(N(c3ccc(-c4cccc5c4oc4ccccc45)cc3)c3cccc4c3oc3c(-c5ccccc5)cccc34)cc2)cc1]

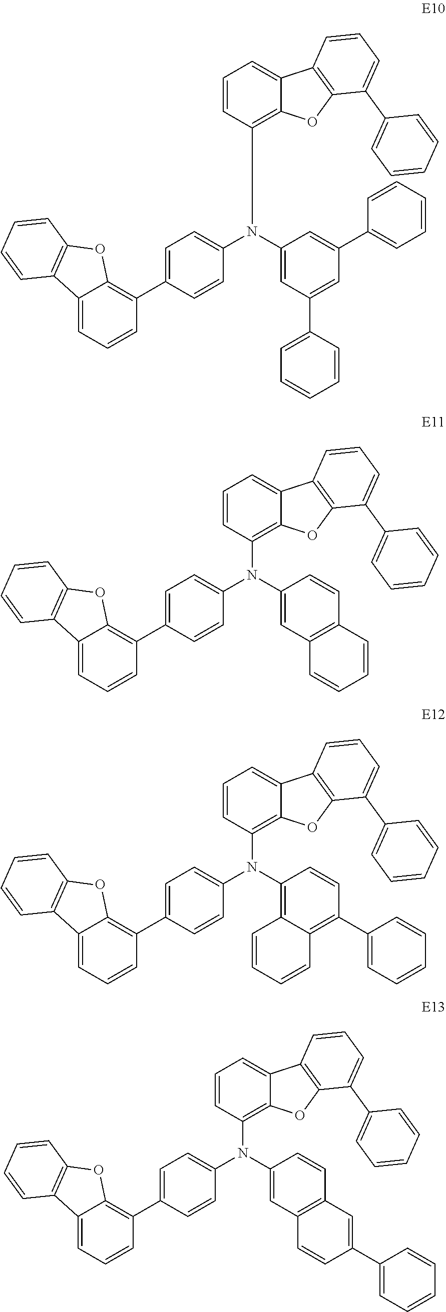 [SMILES: c1ccc(-c2cc(-c3ccccc3)cc(N(c3ccc(-c4cccc5c4oc4ccccc45)cc3)c3cccc4c3oc3c(-c5ccccc5)cccc34)c2)cc1 ; c1ccc(-c2cccc3c2oc2c(N(c4ccc(-c5cccc6c5oc5ccccc56)cc4)c4ccc5ccccc5c4)cccc23)cc1 ; c1ccc(-c2ccc(N(c3ccc(-c4cccc5c4oc4ccccc45)cc3)c3cccc4c3oc3c(-c5ccccc5)cccc34)c3ccccc23)cc1 ; c1ccc(-c2ccc3cc(N(c4ccc(-c5cccc6c5oc5ccccc56)cc4)c4cccc5c4oc4c(-c6ccccc6)cccc45)ccc3c2)cc1]

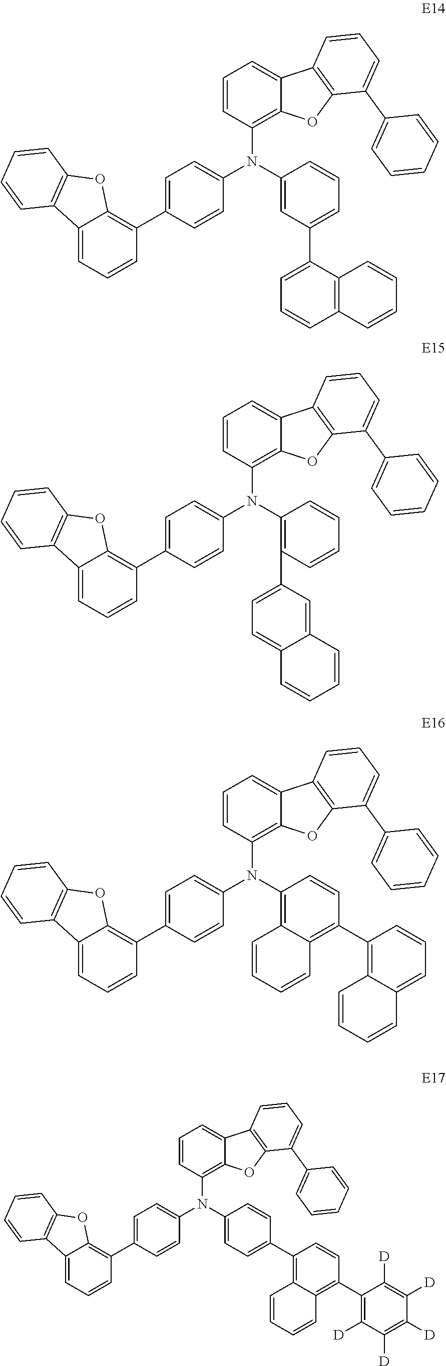

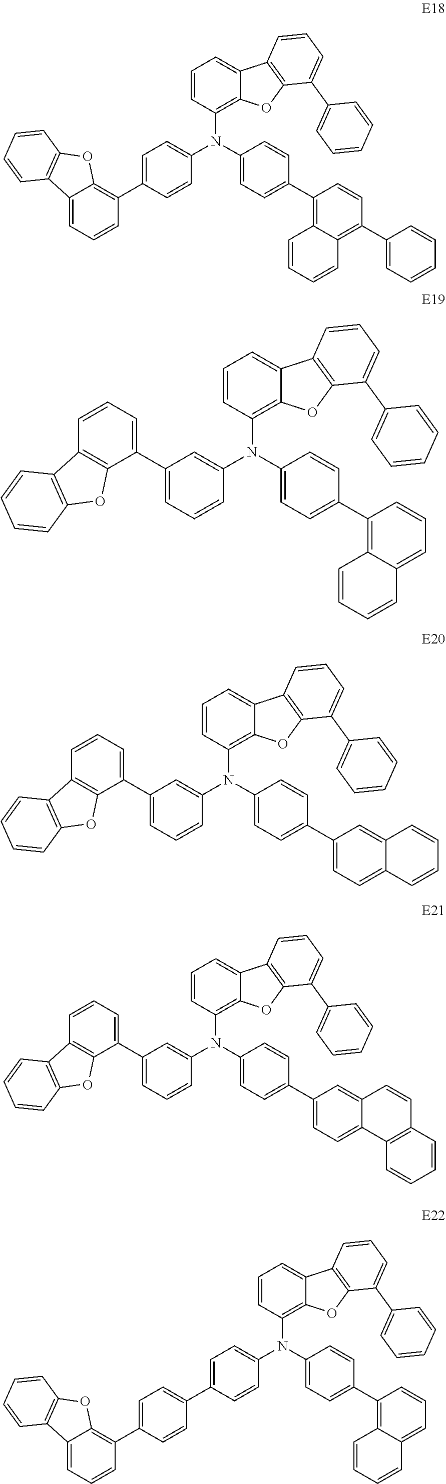 [SMILES: c1ccc(-c2ccc(-c3ccc(N(c4ccc(-c5cccc6c5oc5ccccc56)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5cccc6c5oc5ccccc56)c4)cccc23)cc1 ; c1ccc(-c2cccc3c2oc2c(N(c4ccc(-c5ccc6ccccc6c5)cc4)c4cccc(-c5cccc6c5oc5ccccc56)c4)cccc23)cc1 ; c1ccc(-c2cccc3c2oc2c(N(c4ccc(-c5ccc6c(ccc7ccccc76)c5)cc4)c4cccc(-c5cccc6c5oc5ccccc56)c4)cccc23)cc1 ; c1ccc(-c2cccc3c2oc2c(N(c4ccc(-c5ccc(-c6cccc7c6oc6ccccc67)cc5)cc4)c4ccc(-c5cccc6ccccc56)cc4)cccc23)cc1]

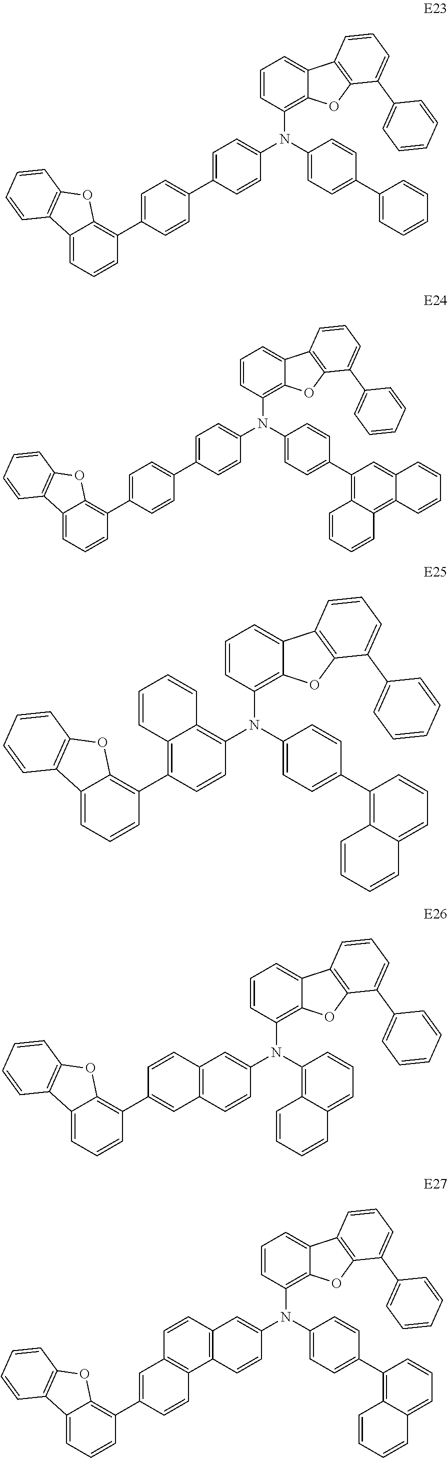 [SMILES: c1ccc(-c2ccc(N(c3ccc(-c4ccc(-c5cccc6c5oc5ccccc56)cc4)cc3)c3cccc4c3oc3c(-c5ccccc5)cccc34)cc2)cc1 ; c1ccc(-c2cccc3c2oc2c(N(c4ccc(-c5ccc(-c6cccc7c6oc6ccccc67)cc5)cc4)c4ccc(-c5cc6ccccc6c6ccccc56)cc4)cccc23)cc1 ; c1ccc(-c2cccc3c2oc2c(N(c4ccc(-c5cccc6ccccc56)cc4)c4ccc(-c5cccc6c5oc5ccccc56)c5ccccc45)cccc23)cc1 ; c1ccc(-c2cccc3c2oc2c(N(c4ccc5cc(-c6cccc7c6oc6ccccc67)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7cccc8c7oc7ccccc78)ccc65)c4)cccc23)cc1]

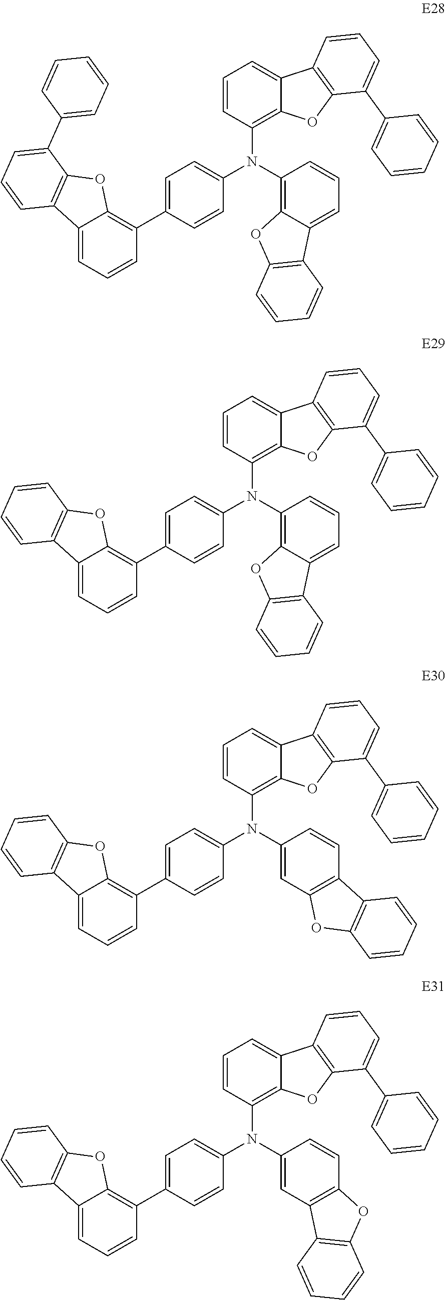 [SMILES: c1ccc(-c2cccc3c2oc2c(-c4ccc(N(c5cccc6c5oc5ccccc56)c5cccc6c5oc5c(-c7ccccc7)cccc56)cc4)cccc23)cc1 ; c1ccc(-c2cccc3c2oc2c(N(c4ccc(-c5cccc6c5oc5ccccc56)cc4)c4cccc5c4oc4ccccc45)cccc23)cc1 ; c1ccc(-c2cccc3c2oc2c(N(c4ccc(-c5cccc6c5oc5ccccc56)cc4)c4ccc5c(c4)oc4ccccc45)cccc23)cc1 ; c1ccc(-c2cccc3c2oc2c(N(c4ccc(-c5cccc6c5oc5ccccc56)cc4)c4ccc5oc6ccccc6c5c4)cccc23)cc1]

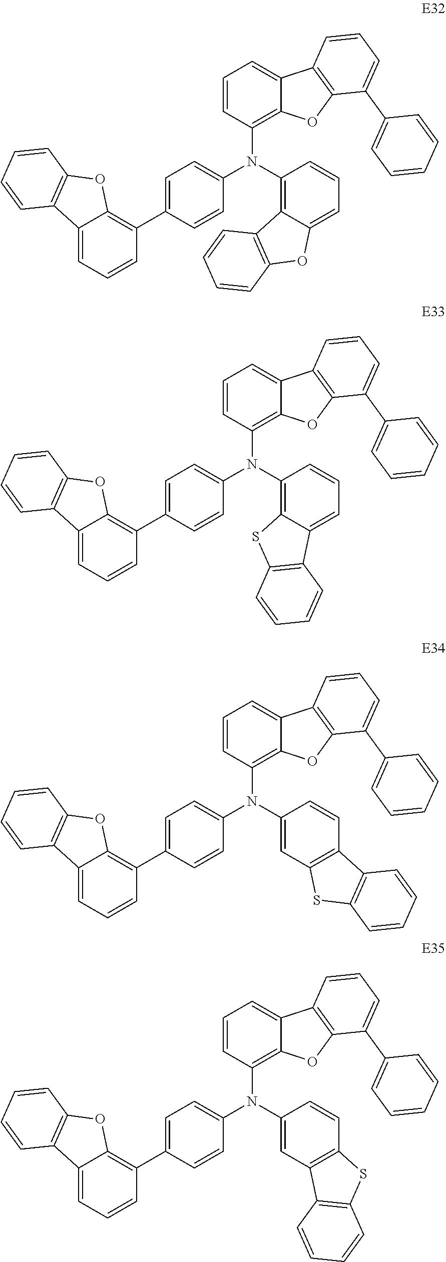

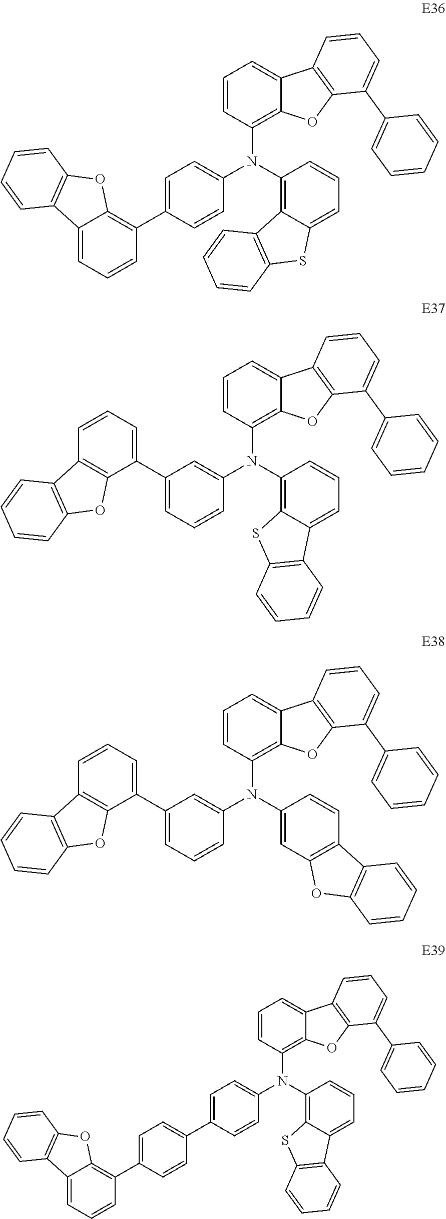 [SMILES: c1ccc(-c2cccc3c2oc2c(N(c4ccc(-c5cccc6c5oc5ccccc56)cc4)c4cccc5sc6ccccc6c45)cccc23)cc1 ; c1ccc(-c2cccc3c2oc2c(N(c4cccc(-c5cccc6c5oc5ccccc56)c4)c4cccc5c4sc4ccccc45)cccc23)cc1 ; c1ccc(-c2cccc3c2oc2c(N(c4cccc(-c5cccc6c5oc5ccccc56)c4)c4ccc5c(c4)oc4ccccc45)cccc23)cc1 ; c1ccc(-c2cccc3c2oc2c(N(c4ccc(-c5ccc(-c6cccc7c6oc6ccccc67)cc5)cc4)c4cccc5c4sc4ccccc45)cccc23)cc1]

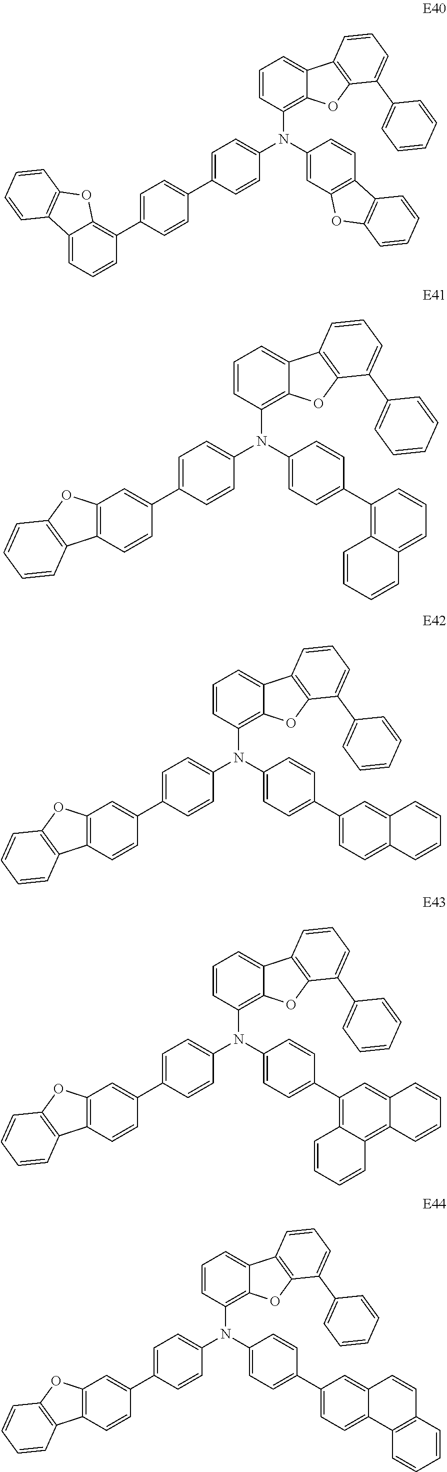 [SMILES: c1ccc(-c2cccc3c2oc2c(N(c4ccc(-c5ccc(-c6cccc7c6oc6ccccc67)cc5)cc4)c4ccc5c(c4)oc4ccccc45)cccc23)cc1 ; c1ccc(-c2cccc3c2oc2c(N(c4ccc(-c5ccc6c(c5)oc5ccccc56)cc4)c4ccc(-c5cccc6ccccc56)cc4)cccc23)cc1 ; c1ccc(-c2cccc3c2oc2c(N(c4ccc(-c5ccc6ccccc6c5)cc4)c4ccc(-c5ccc6c(c5)oc5ccccc56)cc4)cccc23)cc1 ; c1ccc(-c2cccc3c2oc2c(N(c4ccc(-c5ccc6c(c5)oc5ccccc56)cc4)c4ccc(-c5cc6ccccc6c6ccccc56)cc4)cccc23)cc1 ; c1ccc(-c2cccc3c2oc2c(N(c4ccc(-c5ccc6c(ccc7ccccc76)c5)cc4)c4ccc(-c5ccc6c(c5)oc5ccccc56)cc4)cccc23)cc1]

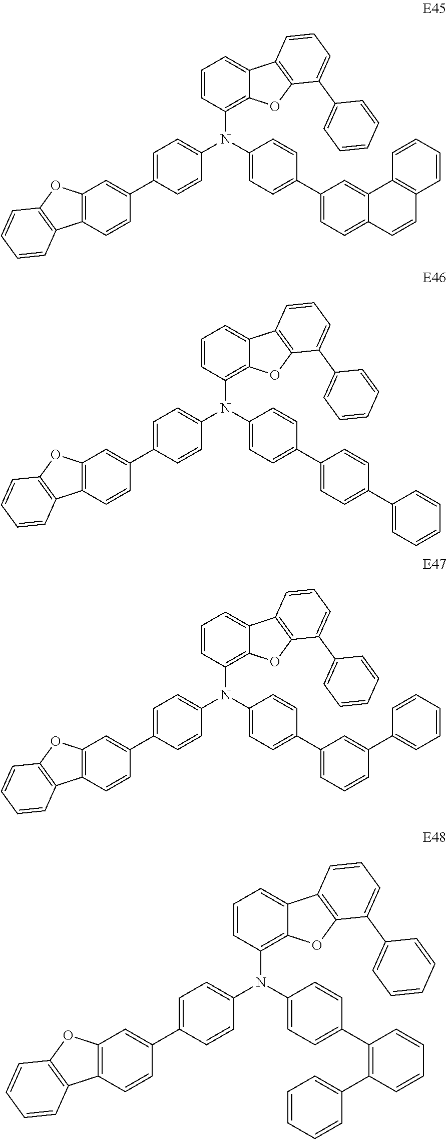 [SMILES: c1ccc(-c2cccc3c2oc2c(N(c4ccc(-c5ccc6c(c5)oc5ccccc56)cc4)c4ccc(-c5ccc6ccc7ccccc7c6c5)cc4)cccc23)cc1 ; c1ccc(-c2ccc(-c3ccc(N(c4ccc(-c5ccc6c(c5)oc5ccccc56)cc4)c4cccc5c4oc4c(-c6ccccc6)cccc45)cc3)cc2)cc1 ; c1ccc(-c2cccc(-c3ccc(N(c4ccc(-c5ccc6c(c5)oc5ccccc56)cc4)c4cccc5c4oc4c(-c6ccccc6)cccc45)cc3)c2)cc1 ; c1ccc(-c2ccccc2-c2ccc(N(c3ccc(-c4ccc5c(c4)oc4ccccc45)cc3)c3cccc4c3oc3c(-c5ccccc5)cccc34)cc2)cc1]

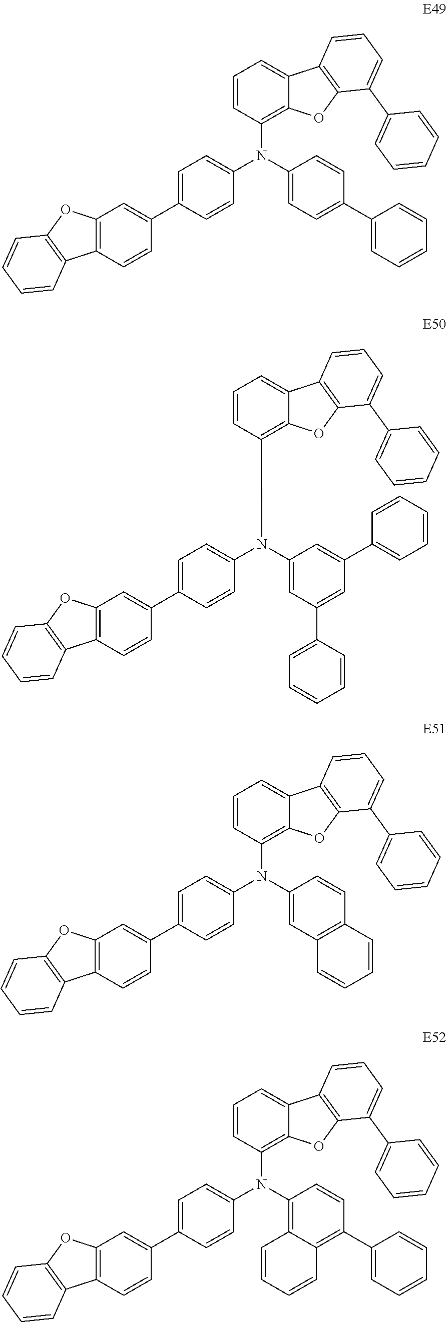 [SMILES: c1ccc(-c2ccc(N(c3ccc(-c4ccc5c(c4)oc4ccccc45)cc3)c3cccc4c3oc3c(-c5ccccc5)cccc34)cc2)cc1 ; c1ccc(-c2cc(-c3ccccc3)cc(N(c3ccc(-c4ccc5c(c4)oc4ccccc45)cc3)c3cccc4c3oc3c(-c5ccccc5)cccc34)c2)cc1 ; c1ccc(-c2cccc3c2oc2c(N(c4ccc(-c5ccc6c(c5)oc5ccccc56)cc4)c4ccc5ccccc5c4)cccc23)cc1 ; c1ccc(-c2ccc(N(c3ccc(-c4ccc5c(c4)oc4ccccc45)cc3)c3cccc4c3oc3c(-c5ccccc5)cccc34)c3ccccc23)cc1]

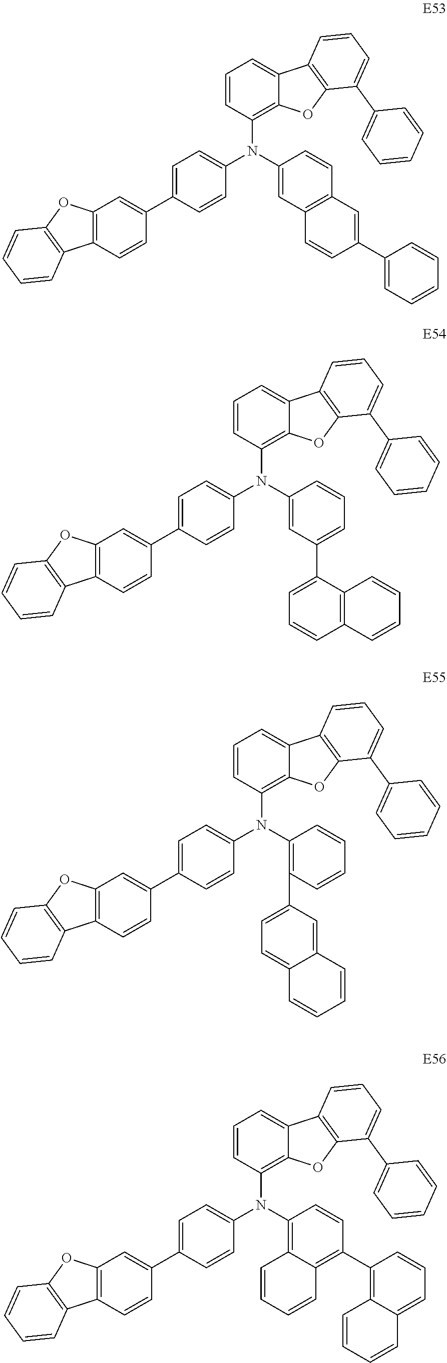 [SMILES: c1ccc(-c2ccc3cc(N(c4ccc(-c5ccc6c(c5)oc5ccccc56)cc4)c4cccc5c4oc4c(-c6ccccc6)cccc45)ccc3c2)cc1 ; c1ccc(-c2cccc3c2oc2c(N(c4ccc(-c5ccc6c(c5)oc5ccccc56)cc4)c4cccc(-c5cccc6ccccc56)c4)cccc23)cc1 ; c1ccc(-c2cccc3c2oc2c(N(c4ccc(-c5ccc6c(c5)oc5ccccc56)cc4)c4ccccc4-c4ccc5ccccc5c4)cccc23)cc1 ; c1ccc(-c2cccc3c2oc2c(N(c4ccc(-c5ccc6c(c5)oc5ccccc56)cc4)c4ccc(-c5cccc6ccccc56)c5ccccc45)cccc23)cc1]

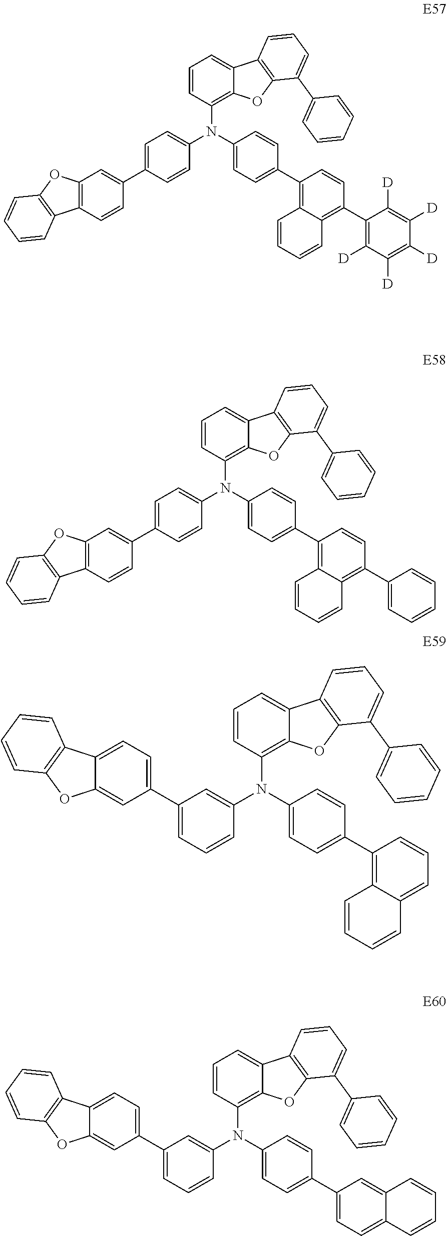 [SMILES: [2H]c1c([2H])c([2H])c(-c2ccc(-c3ccc(N(c4ccc(-c5ccc6c(c5)oc5ccccc56)cc4)c4cccc5c4oc4c(-c6ccccc6)cccc45)cc3)c3ccccc23)c([2H])c1[2H] ; c1ccc(-c2ccc(-c3ccc(N(c4ccc(-c5ccc6c(c5)oc5ccccc56)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5ccc6c(c5)oc5ccccc56)c4)cccc23)cc1 ; c1ccc(-c2cccc3c2oc2c(N(c4ccc(-c5ccc6ccccc6c5)cc4)c4cccc(-c5ccc6c(c5)oc5ccccc56)c4)cccc23)cc1]

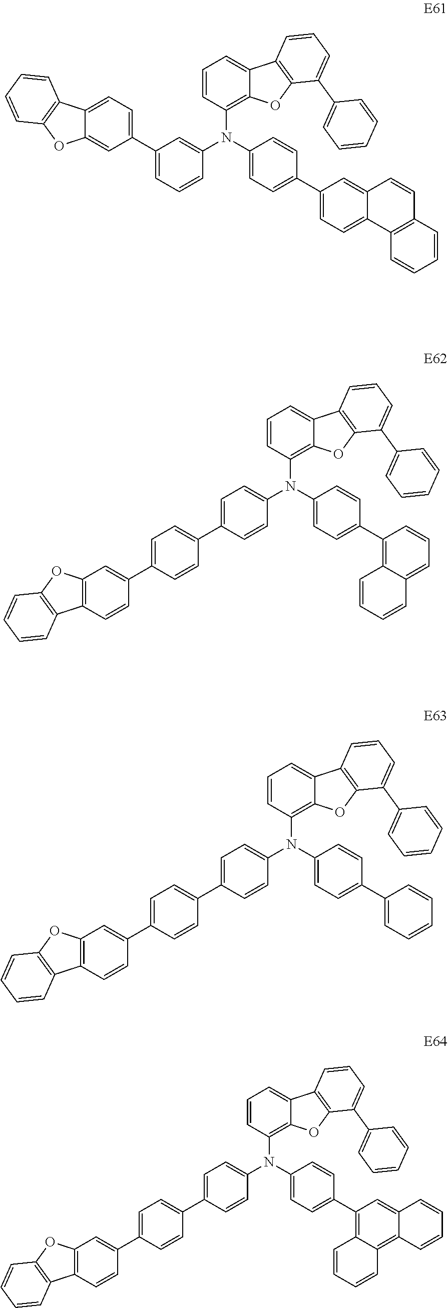 [SMILES: c1ccc(-c2cccc3c2oc2c(N(c4ccc(-c5ccc6c(ccc7ccccc76)c5)cc4)c4cccc(-c5ccc6c(c5)oc5ccccc56)c4)cccc23)cc1 ; c1ccc(-c2cccc3c2oc2c(N(c4ccc(-c5ccc(-c6ccc7c(c6)oc6ccccc67)cc5)cc4)c4ccc(-c5cccc6ccccc56)cc4)cccc23)cc1 ; c1ccc(-c2ccc(N(c3ccc(-c4ccc(-c5ccc6c(c5)oc5ccccc56)cc4)cc3)c3cccc4c3oc3c(-c5ccccc5)cccc34)cc2)cc1 ; c1ccc(-c2cccc3c2oc2c(N(c4ccc(-c5ccc(-c6ccc7c(c6)oc6ccccc67)cc5)cc4)c4ccc(-c5cc6ccccc6c6ccccc56)cc4)cccc23)cc1]

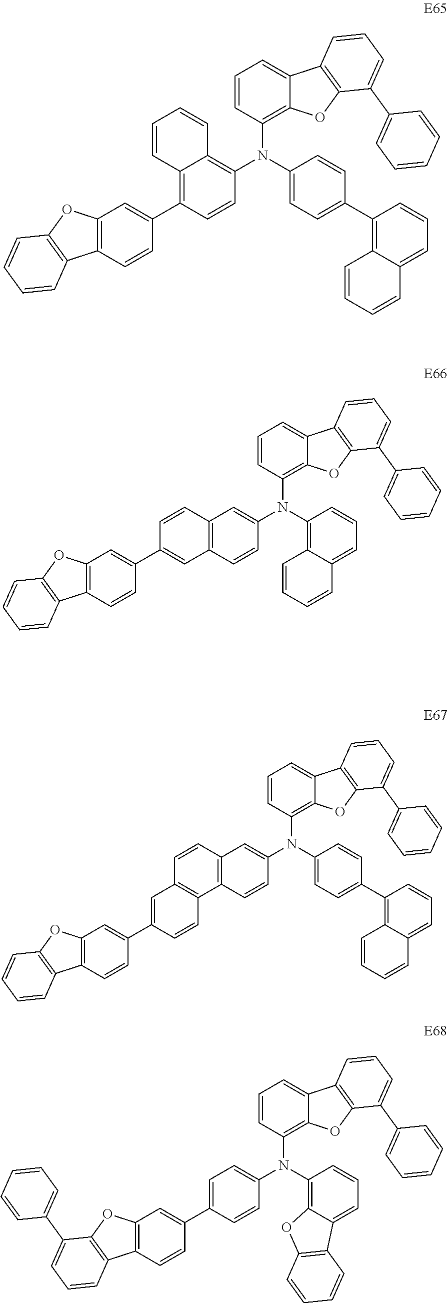 [SMILES: c1ccc(-c2cccc3c2oc2c(N(c4ccc(-c5cccc6ccccc56)cc4)c4ccc(-c5ccc6c(c5)oc5ccccc56)c5ccccc45)cccc23)cc1 ; c1ccc(-c2cccc3c2oc2c(N(c4ccc5cc(-c6ccc7c(c6)oc6ccccc67)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7ccc8c(c7)oc7ccccc78)ccc65)c4)cccc23)cc1 ; c1ccc(-c2cccc3c2oc2cc(-c4ccc(N(c5cccc6c5oc5ccccc56)c5cccc6c5oc5c(-c7ccccc7)cccc56)cc4)ccc23)cc1]

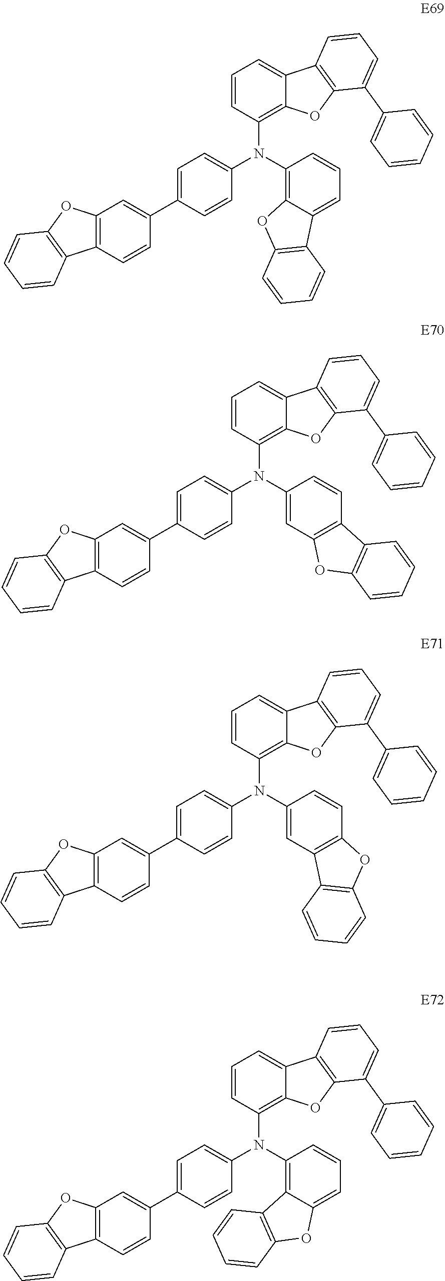 [SMILES: c1ccc(-c2cccc3c2oc2c(N(c4ccc(-c5ccc6c(c5)oc5ccccc56)cc4)c4cccc5c4oc4ccccc45)cccc23)cc1 ; c1ccc(-c2cccc3c2oc2c(N(c4ccc(-c5ccc6c(c5)oc5ccccc56)cc4)c4ccc5c(c4)oc4ccccc45)cccc23)cc1 ; c1ccc(-c2cccc3c2oc2c(N(c4ccc(-c5ccc6c(c5)oc5ccccc56)cc4)c4ccc5oc6ccccc6c5c4)cccc23)cc1 ; c1ccc(-c2cccc3c2oc2c(N(c4ccc(-c5ccc6c(c5)oc5ccccc56)cc4)c4cccc5oc6ccccc6c45)cccc23)cc1]

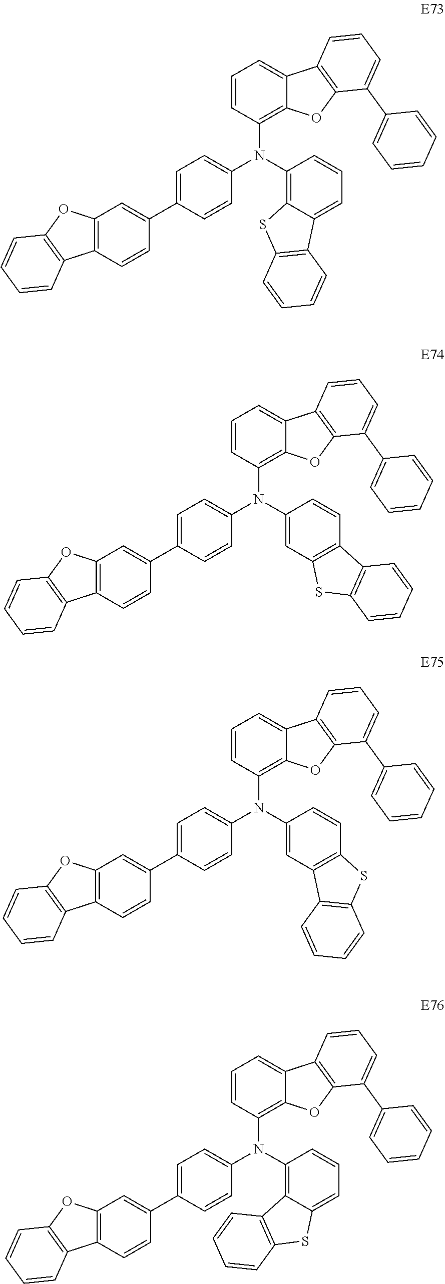 [SMILES: c1ccc(-c2cccc3c2oc2c(N(c4ccc(-c5ccc6c(c5)oc5ccccc56)cc4)c4cccc5c4sc4ccccc45)cccc23)cc1 ; c1ccc(-c2cccc3c2oc2c(N(c4ccc(-c5ccc6c(c5)oc5ccccc56)cc4)c4ccc5c(c4)sc4ccccc45)cccc23)cc1 ; c1ccc(-c2cccc3c2oc2c(N(c4ccc(-c5ccc6c(c5)oc5ccccc56)cc4)c4ccc5sc6ccccc6c5c4)cccc23)cc1 ; c1ccc(-c2cccc3c2oc2c(N(c4ccc(-c5ccc6c(c5)oc5ccccc56)cc4)c4cccc5sc6ccccc6c45)cccc23)cc1]

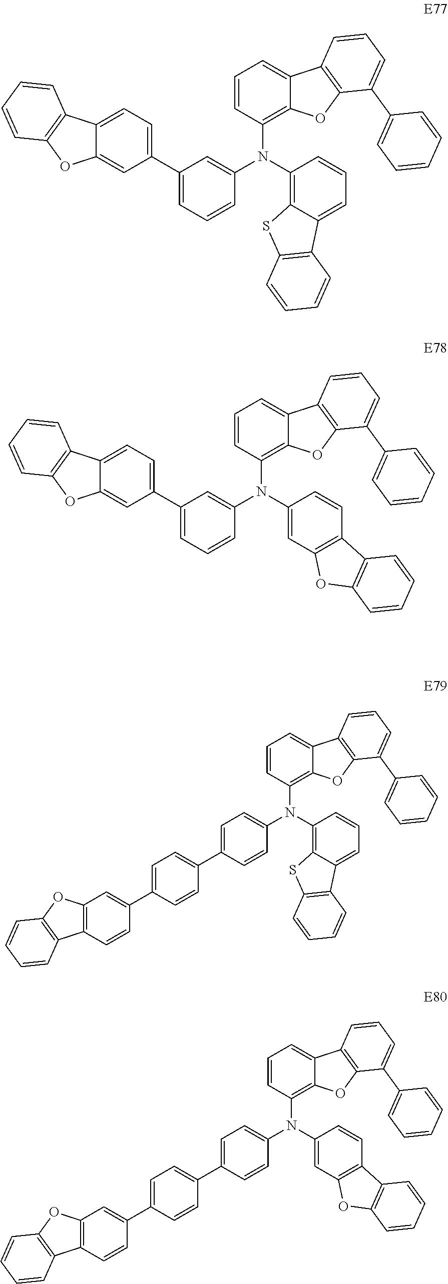 [SMILES: c1ccc(-c2cccc3c2oc2c(N(c4cccc(-c5ccc6c(c5)oc5ccccc56)c4)c4cccc5c4sc4ccccc45)cccc23)cc1 ; c1ccc(-c2cccc3c2oc2c(N(c4cccc(-c5ccc6c(c5)oc5ccccc56)c4)c4ccc5c(c4)oc4ccccc45)cccc23)cc1 ; c1ccc(-c2cccc3c2oc2c(N(c4ccc(-c5ccc(-c6ccc7c(c6)oc6ccccc67)cc5)cc4)c4cccc5c4sc4ccccc45)cccc23)cc1 ; c1ccc(-c2cccc3c2oc2c(N(c4ccc(-c5ccc(-c6ccc7c(c6)oc6ccccc67)cc5)cc4)c4ccc5c(c4)oc4ccccc45)cccc23)cc1]

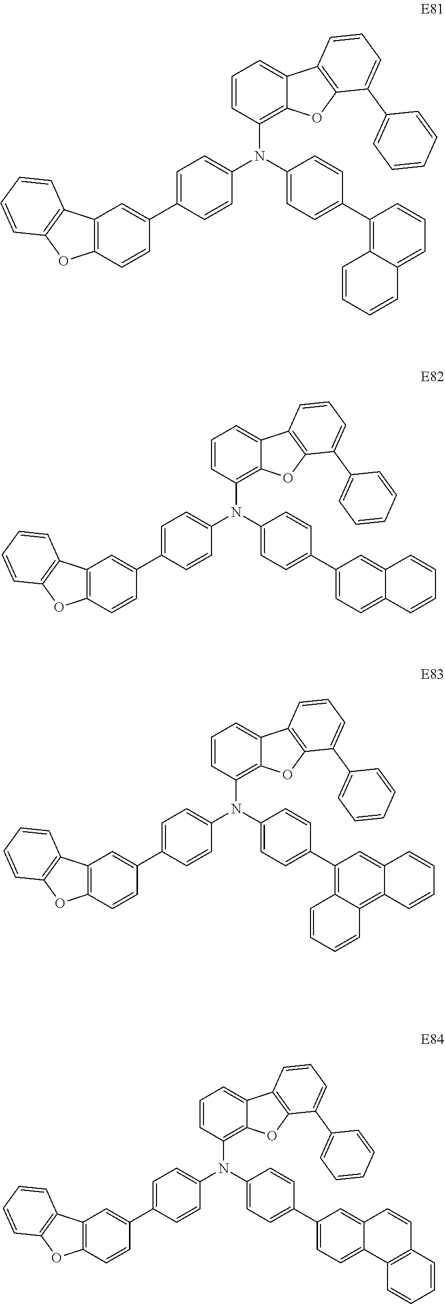 [SMILES: c1ccc(-c2cccc3c2oc2c(N(c4ccc(-c5ccc6oc7ccccc7c6c5)cc4)c4ccc(-c5cccc6ccccc56)cc4)cccc23)cc1 ; c1ccc(-c2cccc3c2oc2c(N(c4ccc(-c5ccc6ccccc6c5)cc4)c4ccc(-c5ccc6oc7ccccc7c6c5)cc4)cccc23)cc1 ; c1ccc(-c2cccc3c2oc2c(N(c4ccc(-c5ccc6oc7ccccc7c6c5)cc4)c4ccc(-c5cc6ccccc6c6ccccc56)cc4)cccc23)cc1 ; c1ccc(-c2cccc3c2oc2c(N(c4ccc(-c5ccc6c(ccc7ccccc76)c5)cc4)c4ccc(-c5ccc6oc7ccccc7c6c5)cc4)cccc23)cc1]

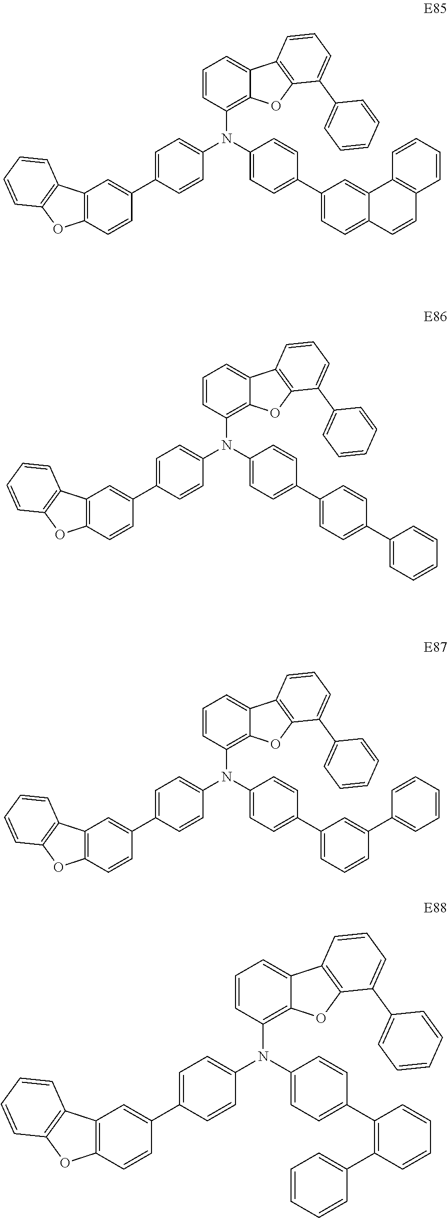 [SMILES: c1ccc(-c2cccc3c2oc2c(N(c4ccc(-c5ccc6ccc7ccccc7c6c5)cc4)c4ccc(-c5ccc6oc7ccccc7c6c5)cc4)cccc23)cc1 ; c1ccc(-c2ccc(-c3ccc(N(c4ccc(-c5ccc6oc7ccccc7c6c5)cc4)c4cccc5c4oc4c(-c6ccccc6)cccc45)cc3)cc2)cc1 ; c1ccc(-c2cccc(-c3ccc(N(c4ccc(-c5ccc6oc7ccccc7c6c5)cc4)c4cccc5c4oc4c(-c6ccccc6)cccc45)cc3)c2)cc1 ; c1ccc(-c2ccccc2-c2ccc(N(c3ccc(-c4ccc5oc6ccccc6c5c4)cc3)c3cccc4c3oc3c(-c5ccccc5)cccc34)cc2)cc1]

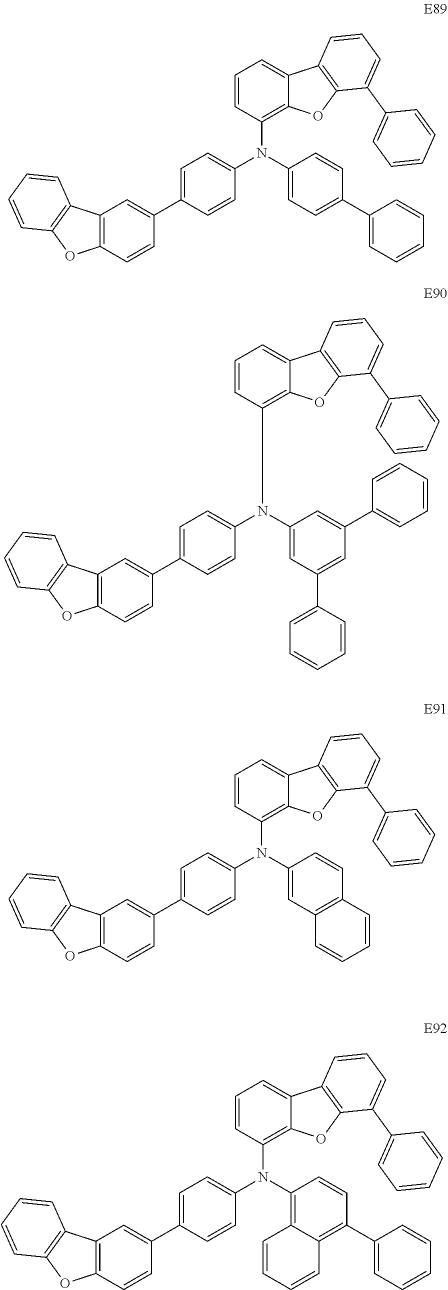 [SMILES: c1ccc(-c2ccc(N(c3ccc(-c4ccc5oc6ccccc6c5c4)cc3)c3cccc4c3oc3c(-c5ccccc5)cccc34)cc2)cc1 ; c1ccc(-c2cc(-c3ccccc3)cc(N(c3ccc(-c4ccc5oc6ccccc6c5c4)cc3)c3cccc4c3oc3c(-c5ccccc5)cccc34)c2)cc1 ; c1ccc(-c2cccc3c2oc2c(N(c4ccc(-c5ccc6oc7ccccc7c6c5)cc4)c4ccc5ccccc5c4)cccc23)cc1 ; c1ccc(-c2ccc(N(c3ccc(-c4ccc5oc6ccccc6c5c4)cc3)c3cccc4c3oc3c(-c5ccccc5)cccc34)c3ccccc23)cc1]

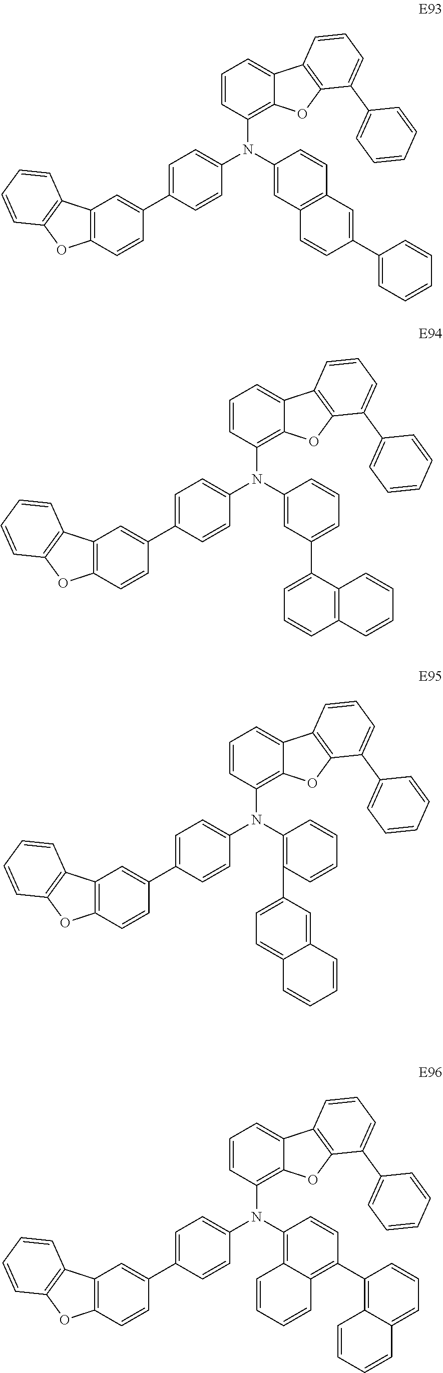 [SMILES: c1ccc(-c2ccc3cc(N(c4ccc(-c5ccc6oc7ccccc7c6c5)cc4)c4cccc5c4oc4c(-c6ccccc6)cccc45)ccc3c2)cc1 ; c1ccc(-c2cccc3c2oc2c(N(c4ccc(-c5ccc6oc7ccccc7c6c5)cc4)c4cccc(-c5cccc6ccccc56)c4)cccc23)cc1 ; c1ccc(-c2cccc3c2oc2c(N(c4ccc(-c5ccc6oc7ccccc7c6c5)cc4)c4ccccc4-c4ccc5ccccc5c4)cccc23)cc1 ; c1ccc(-c2cccc3c2oc2c(N(c4ccc(-c5ccc6oc7ccccc7c6c5)cc4)c4ccc(-c5cccc6ccccc56)c5ccccc45)cccc23)cc1]

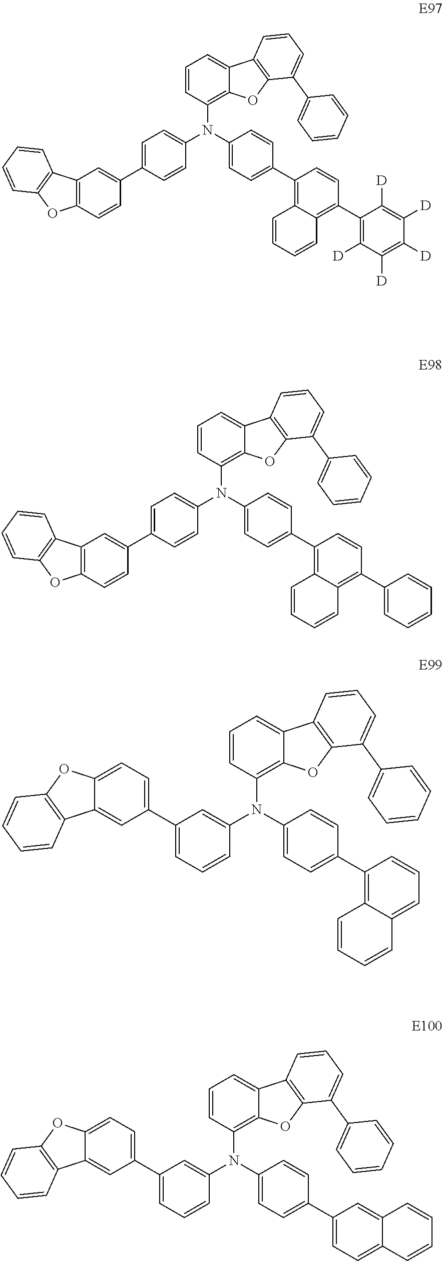 [SMILES: [2H]c1c([2H])c([2H])c(-c2ccc(-c3ccc(N(c4ccc(-c5ccc6oc7ccccc7c6c5)cc4)c4cccc5c4oc4c(-c6ccccc6)cccc45)cc3)c3ccccc23)c([2H])c1[2H] ; c1ccc(-c2ccc(-c3ccc(N(c4ccc(-c5ccc6oc7ccccc7c6c5)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5ccc6oc7ccccc7c6c5)c4)cccc23)cc1 ; c1ccc(-c2cccc3c2oc2c(N(c4ccc(-c5ccc6ccccc6c5)cc4)c4cccc(-c5ccc6oc7ccccc7c6c5)c4)cccc23)cc1]

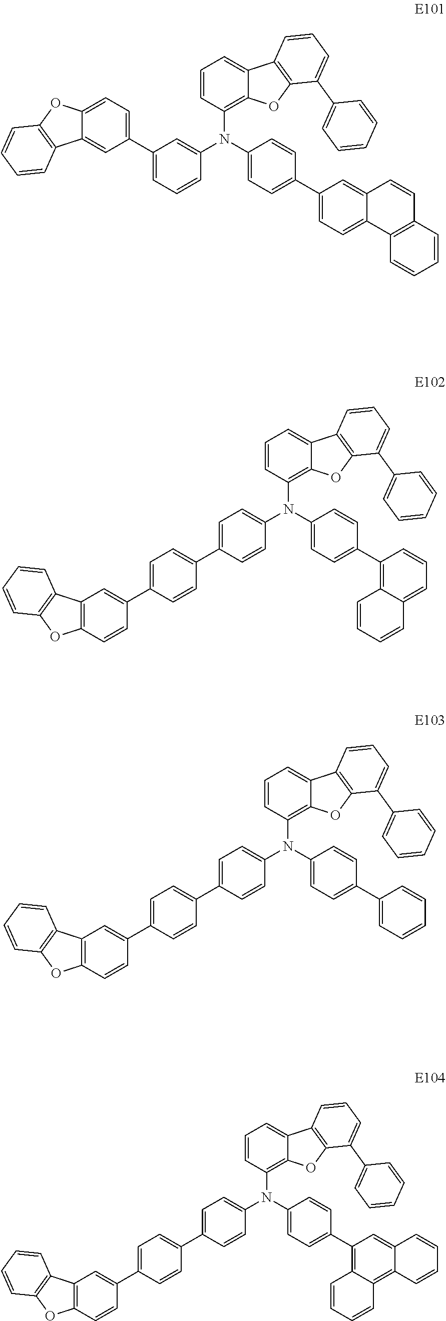 [SMILES: c1ccc(-c2cccc3c2oc2c(N(c4ccc(-c5ccc6c(ccc7ccccc76)c5)cc4)c4cccc(-c5ccc6oc7ccccc7c6c5)c4)cccc23)cc1 ; c1ccc(-c2cccc3c2oc2c(N(c4ccc(-c5ccc(-c6ccc7oc8ccccc8c7c6)cc5)cc4)c4ccc(-c5cccc6ccccc56)cc4)cccc23)cc1 ; c1ccc(-c2ccc(N(c3ccc(-c4ccc(-c5ccc6oc7ccccc7c6c5)cc4)cc3)c3cccc4c3oc3c(-c5ccccc5)cccc34)cc2)cc1 ; c1ccc(-c2cccc3c2oc2c(N(c4ccc(-c5ccc(-c6ccc7oc8ccccc8c7c6)cc5)cc4)c4ccc(-c5cc6ccccc6c6ccccc56)cc4)cccc23)cc1]

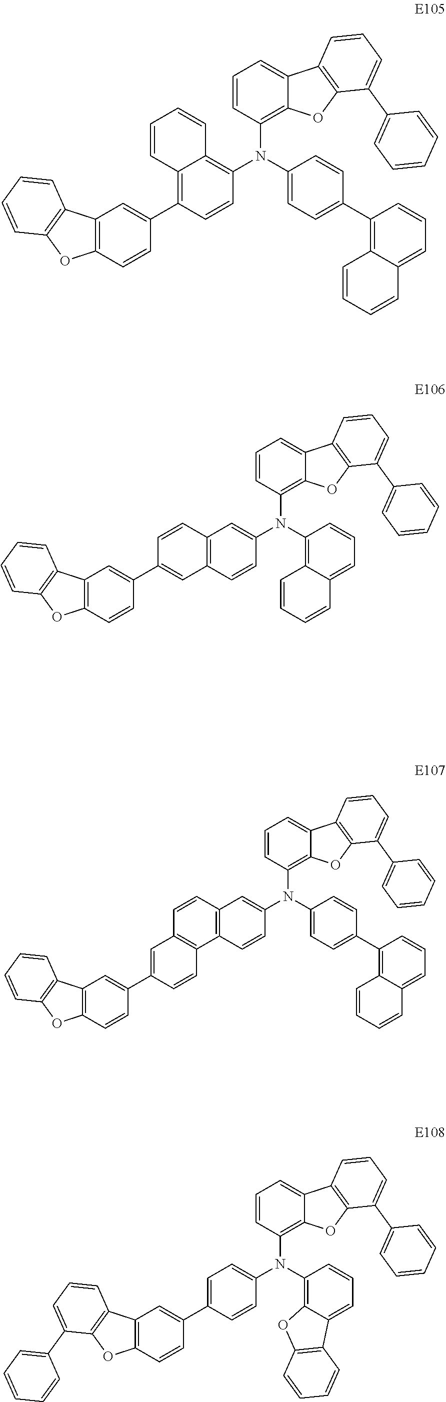 [SMILES: c1ccc(-c2cccc3c2oc2c(N(c4ccc(-c5cccc6ccccc56)cc4)c4ccc(-c5ccc6oc7ccccc7c6c5)c5ccccc45)cccc23)cc1 ; c1ccc(-c2cccc3c2oc2c(N(c4ccc5cc(-c6ccc7oc8ccccc8c7c6)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7ccc8oc9ccccc9c8c7)ccc65)c4)cccc23)cc1 ; c1ccc(-c2cccc3c2oc2ccc(-c4ccc(N(c5cccc6c5oc5ccccc56)c5cccc6c5oc5c(-c7ccccc7)cccc56)cc4)cc23)cc1]

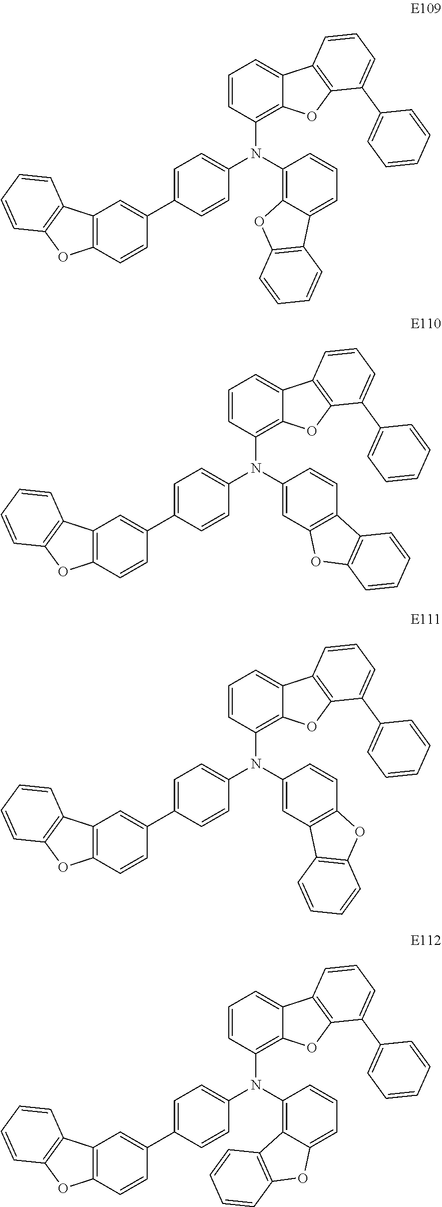 [SMILES: c1ccc(-c2cccc3c2oc2c(N(c4ccc(-c5ccc6oc7ccccc7c6c5)cc4)c4cccc5c4oc4ccccc45)cccc23)cc1 ; c1ccc(-c2cccc3c2oc2c(N(c4ccc(-c5ccc6oc7ccccc7c6c5)cc4)c4ccc5c(c4)oc4ccccc45)cccc23)cc1 ; c1ccc(-c2cccc3c2oc2c(N(c4ccc(-c5ccc6oc7ccccc7c6c5)cc4)c4ccc5oc6ccccc6c5c4)cccc23)cc1 ; c1ccc(-c2cccc3c2oc2c(N(c4ccc(-c5ccc6oc7ccccc7c6c5)cc4)c4cccc5oc6ccccc6c45)cccc23)cc1]

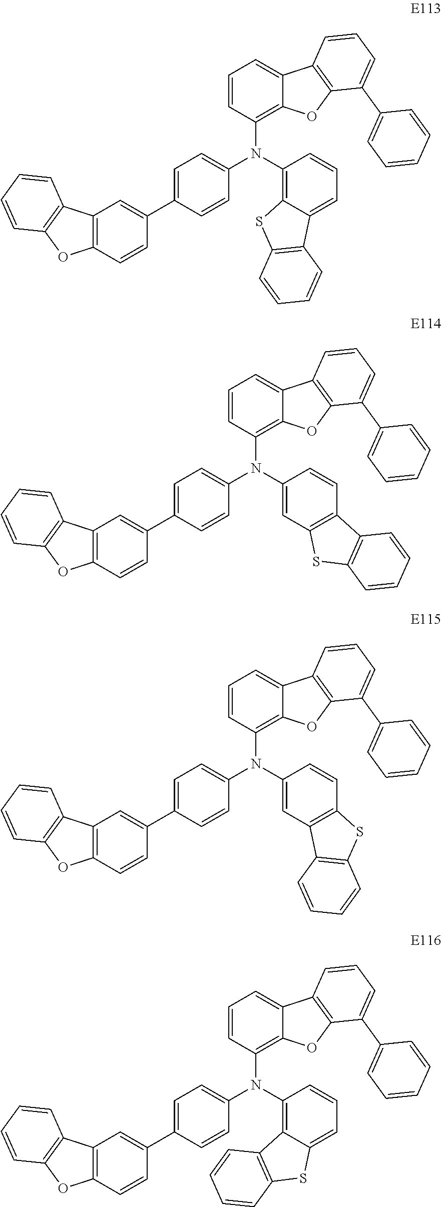 [SMILES: c1ccc(-c2cccc3c2oc2c(N(c4ccc(-c5ccc6oc7ccccc7c6c5)cc4)c4cccc5c4sc4ccccc45)cccc23)cc1 ; c1ccc(-c2cccc3c2oc2c(N(c4ccc(-c5ccc6oc7ccccc7c6c5)cc4)c4ccc5c(c4)sc4ccccc45)cccc23)cc1 ; c1ccc(-c2cccc3c2oc2c(N(c4ccc(-c5ccc6oc7ccccc7c6c5)cc4)c4ccc5sc6ccccc6c5c4)cccc23)cc1 ; c1ccc(-c2cccc3c2oc2c(N(c4ccc(-c5ccc6oc7ccccc7c6c5)cc4)c4cccc5sc6ccccc6c45)cccc23)cc1]

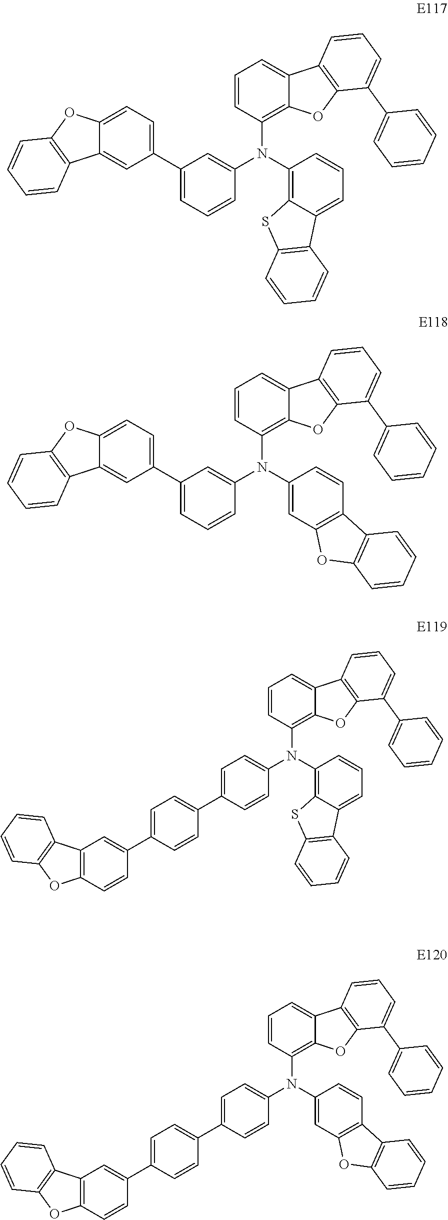 [SMILES: c1ccc(-c2cccc3c2oc2c(N(c4cccc(-c5ccc6oc7ccccc7c6c5)c4)c4cccc5c4sc4ccccc45)cccc23)cc1 ; c1ccc(-c2cccc3c2oc2c(N(c4cccc(-c5ccc6oc7ccccc7c6c5)c4)c4ccc5c(c4)oc4ccccc45)cccc23)cc1 ; c1ccc(-c2cccc3c2oc2c(N(c4ccc(-c5ccc(-c6ccc7oc8ccccc8c7c6)cc5)cc4)c4cccc5c4sc4ccccc45)cccc23)cc1 ; c1ccc(-c2cccc3c2oc2c(N(c4ccc(-c5ccc(-c6ccc7oc8ccccc8c7c6)cc5)cc4)c4ccc5c(c4)oc4ccccc45)cccc23)cc1]

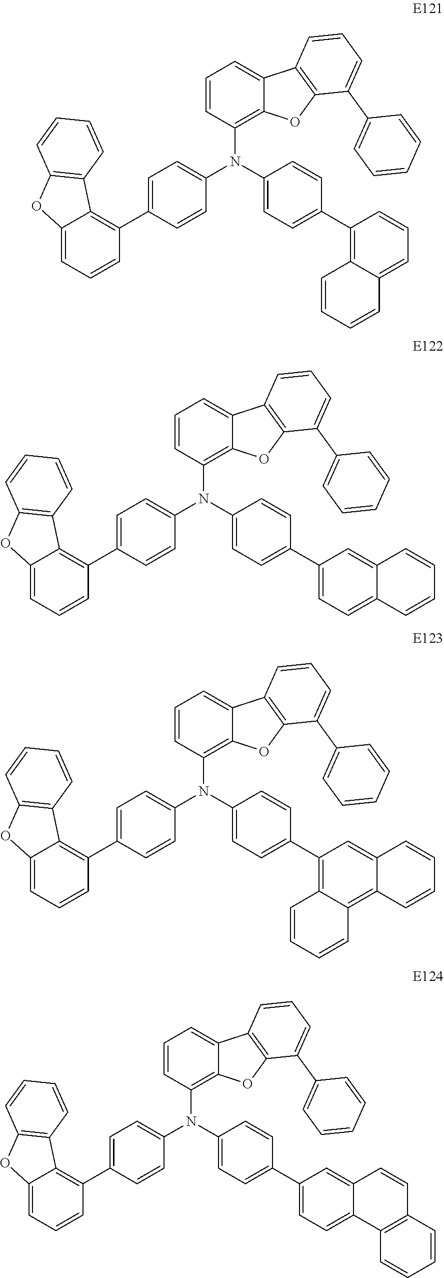 [SMILES: c1ccc(-c2cccc3c2oc2c(N(c4ccc(-c5cccc6ccccc56)cc4)c4ccc(-c5cccc6oc7ccccc7c56)cc4)cccc23)cc1 ; c1ccc(-c2cccc3c2oc2c(N(c4ccc(-c5ccc6ccccc6c5)cc4)c4ccc(-c5cccc6oc7ccccc7c56)cc4)cccc23)cc1 ; c1ccc(-c2cccc3c2oc2c(N(c4ccc(-c5cc6ccccc6c6ccccc56)cc4)c4ccc(-c5cccc6oc7ccccc7c56)cc4)cccc23)cc1 ; c1ccc(-c2cccc3c2oc2c(N(c4ccc(-c5ccc6c(ccc7ccccc76)c5)cc4)c4ccc(-c5cccc6oc7ccccc7c56)cc4)cccc23)cc1]

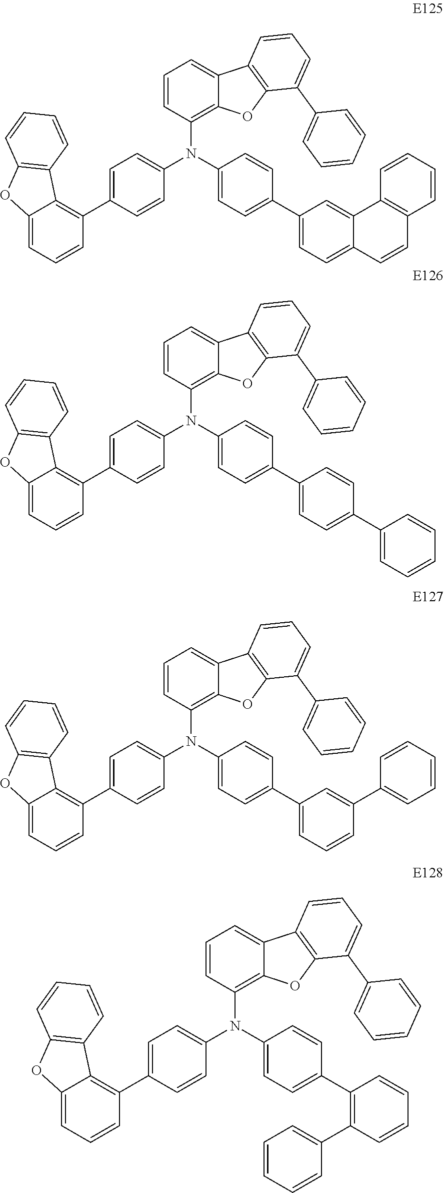 [SMILES: c1ccc(-c2cccc3c2oc2c(N(c4ccc(-c5ccc6ccc7ccccc7c6c5)cc4)c4ccc(-c5cccc6oc7ccccc7c56)cc4)cccc23)cc1 ; c1ccc(-c2ccc(-c3ccc(N(c4ccc(-c5cccc6oc7ccccc7c56)cc4)c4cccc5c4oc4c(-c6ccccc6)cccc45)cc3)cc2)cc1 ; c1ccc(-c2cccc(-c3ccc(N(c4ccc(-c5cccc6oc7ccccc7c56)cc4)c4cccc5c4oc4c(-c6ccccc6)cccc45)cc3)c2)cc1 ; c1ccc(-c2ccccc2-c2ccc(N(c3ccc(-c4cccc5oc6ccccc6c45)cc3)c3cccc4c3oc3c(-c5ccccc5)cccc34)cc2)cc1]

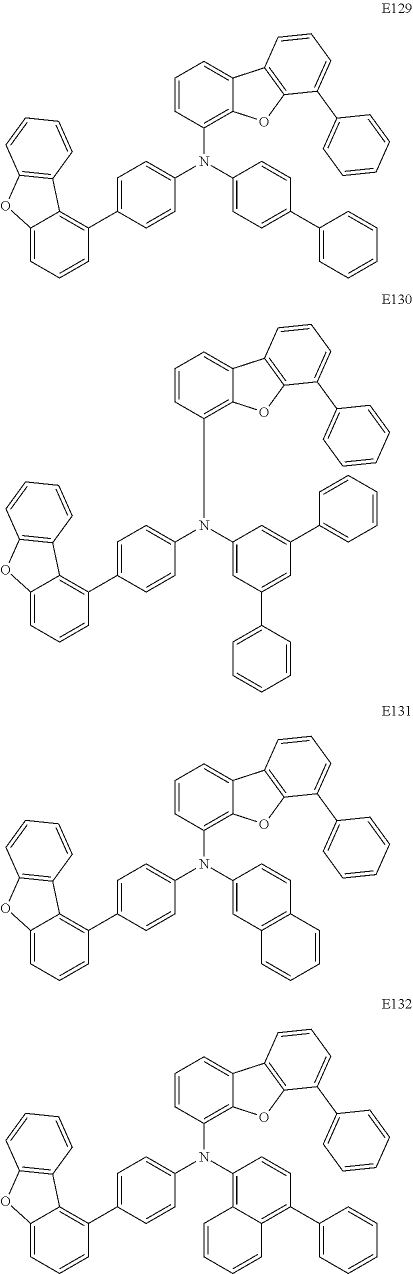 [SMILES: c1ccc(-c2ccc(N(c3ccc(-c4cccc5oc6ccccc6c45)cc3)c3cccc4c3oc3c(-c5ccccc5)cccc34)cc2)cc1 ; c1ccc(-c2cc(-c3ccccc3)cc(N(c3ccc(-c4cccc5oc6ccccc6c45)cc3)c3cccc4c3oc3c(-c5ccccc5)cccc34)c2)cc1 ; c1ccc(-c2cccc3c2oc2c(N(c4ccc(-c5cccc6oc7ccccc7c56)cc4)c4ccc5ccccc5c4)cccc23)cc1 ; c1ccc(-c2ccc(N(c3ccc(-c4cccc5oc6ccccc6c45)cc3)c3cccc4c3oc3c(-c5ccccc5)cccc34)c3ccccc23)cc1]

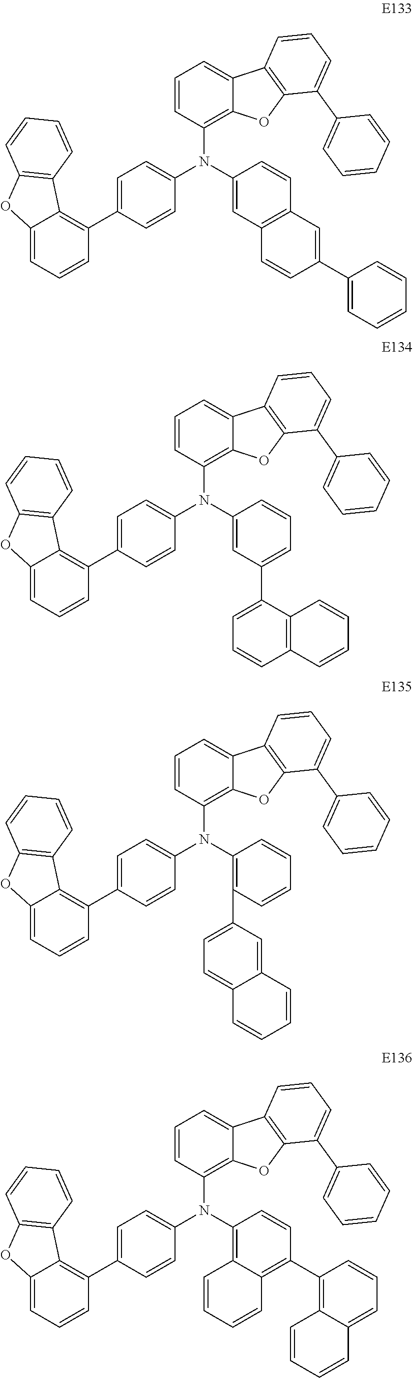

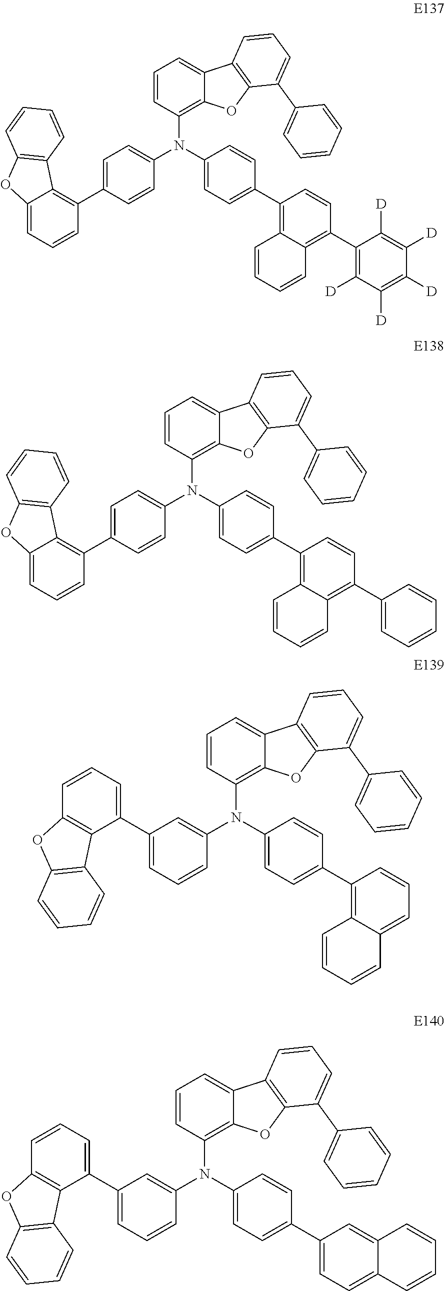 [SMILES: [2H]c1c([2H])c([2H])c(-c2ccc(-c3ccc(N(c4ccc(-c5cccc6oc7ccccc7c56)cc4)c4cccc5c4oc4c(-c6ccccc6)cccc45)cc3)c3ccccc23)c([2H])c1[2H] ; c1ccc(-c2ccc(-c3ccc(N(c4ccc(-c5cccc6oc7ccccc7c56)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5cccc6oc7ccccc7c56)c4)cccc23)cc1 ; c1ccc(-c2cccc3c2oc2c(N(c4ccc(-c5ccc6ccccc6c5)cc4)c4cccc(-c5cccc6oc7ccccc7c56)c4)cccc23)cc1]

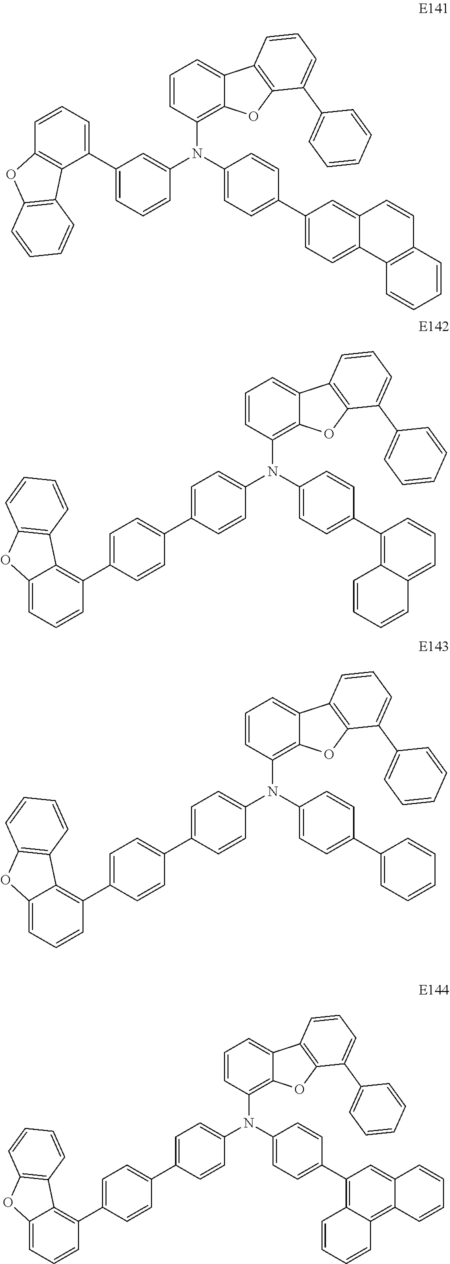 [SMILES: c1ccc(-c2cccc3c2oc2c(N(c4ccc(-c5ccc6c(ccc7ccccc76)c5)cc4)c4cccc(-c5cccc6oc7ccccc7c56)c4)cccc23)cc1 ; c1ccc(-c2cccc3c2oc2c(N(c4ccc(-c5ccc(-c6cccc7oc8ccccc8c67)cc5)cc4)c4ccc(-c5cccc6ccccc56)cc4)cccc23)cc1 ; c1ccc(-c2ccc(N(c3ccc(-c4ccc(-c5cccc6oc7ccccc7c56)cc4)cc3)c3cccc4c3oc3c(-c5ccccc5)cccc34)cc2)cc1 ; c1ccc(-c2cccc3c2oc2c(N(c4ccc(-c5ccc(-c6cccc7oc8ccccc8c67)cc5)cc4)c4ccc(-c5cc6ccccc6c6ccccc56)cc4)cccc23)cc1]

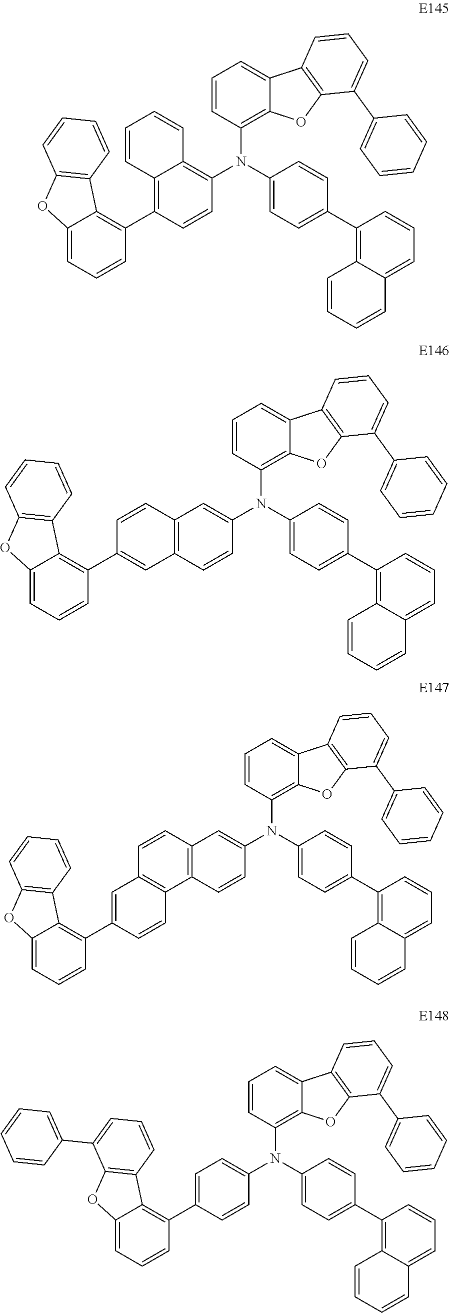 [SMILES: c1ccc(-c2cccc3c2oc2c(N(c4ccc(-c5cccc6ccccc56)cc4)c4ccc(-c5cccc6oc7ccccc7c56)c5ccccc45)cccc23)cc1 ; c1ccc(-c2cccc3c2oc2c(N(c4ccc(-c5cccc6ccccc56)cc4)c4ccc5cc(-c6cccc7oc8ccccc8c67)ccc5c4)cccc23)cc1 ; c1ccc(-c2cccc3c2oc2c(N(c4ccc(-c5cccc6ccccc56)cc4)c4ccc5c(ccc6cc(-c7cccc8oc9ccccc9c78)ccc65)c4)cccc23)cc1 ; c1ccc(-c2cccc3c2oc2c(N(c4ccc(-c5cccc6ccccc56)cc4)c4ccc(-c5cccc6oc7c(-c8ccccc8)cccc7c56)cc4)cccc23)cc1]

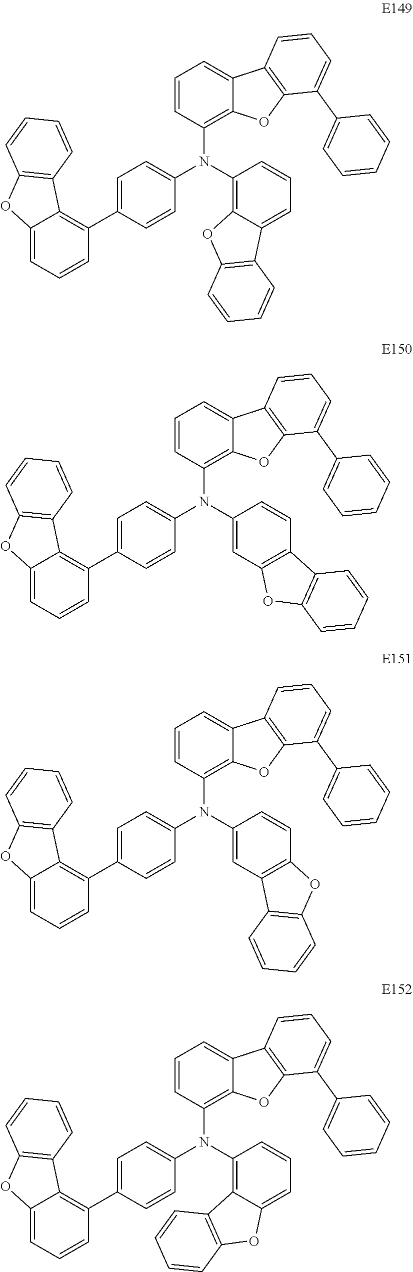

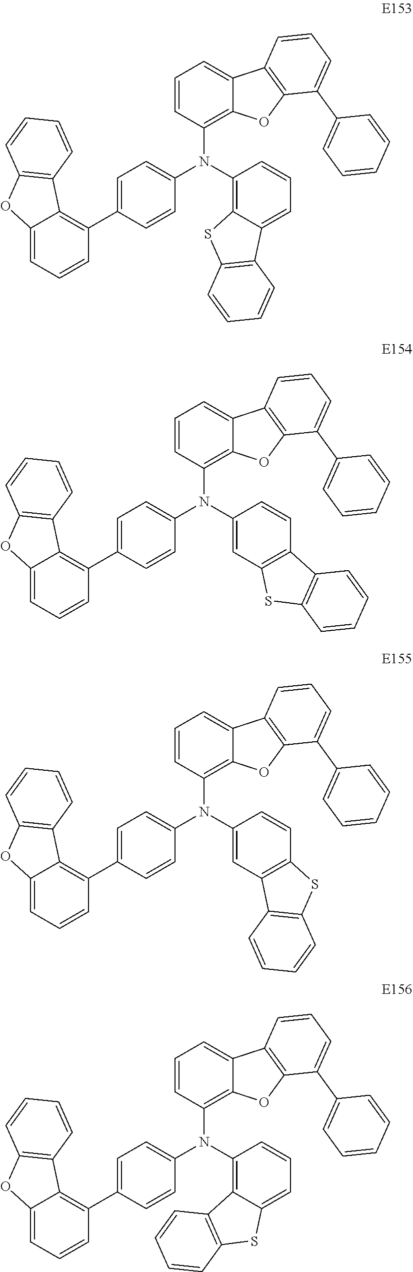

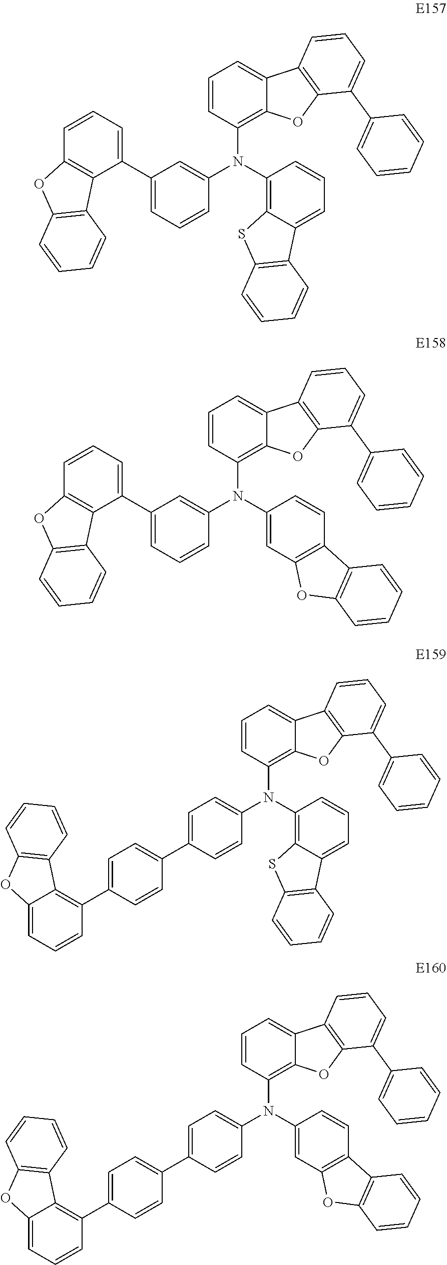 [SMILES: c1ccc(-c2cccc3c2oc2c(N(c4cccc(-c5cccc6oc7ccccc7c56)c4)c4cccc5c4sc4ccccc45)cccc23)cc1 ; c1ccc(-c2cccc3c2oc2c(N(c4cccc(-c5cccc6oc7ccccc7c56)c4)c4ccc5c(c4)oc4ccccc45)cccc23)cc1 ; c1ccc(-c2cccc3c2oc2c(N(c4ccc(-c5ccc(-c6cccc7oc8ccccc8c67)cc5)cc4)c4cccc5c4sc4ccccc45)cccc23)cc1 ; c1ccc(-c2cccc3c2oc2c(N(c4ccc(-c5ccc(-c6cccc7oc8ccccc8c67)cc5)cc4)c4ccc5c(c4)oc4ccccc45)cccc23)cc1]

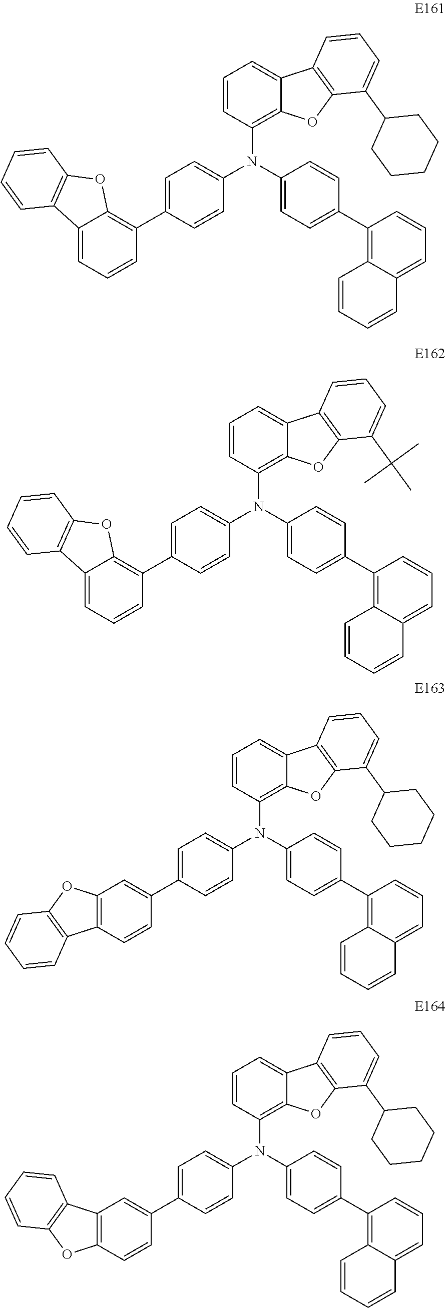

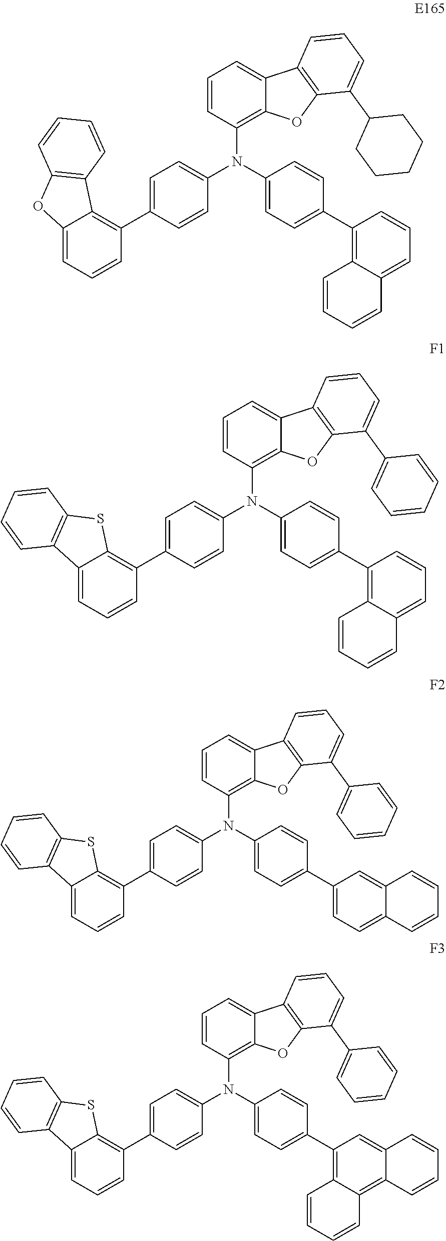 [SMILES: c1ccc2c(-c3ccc(N(c4ccc(-c5cccc6oc7ccccc7c56)cc4)c4cccc5c4oc4c(C6CCCCC6)cccc45)cc3)cccc2c1 ; c1ccc(-c2cccc3c2oc2c(N(c4ccc(-c5cccc6ccccc56)cc4)c4ccc(-c5cccc6c5sc5ccccc56)cc4)cccc23)cc1 ; c1ccc(-c2cccc3c2oc2c(N(c4ccc(-c5ccc6ccccc6c5)cc4)c4ccc(-c5cccc6c5sc5ccccc56)cc4)cccc23)cc1 ; c1ccc(-c2cccc3c2oc2c(N(c4ccc(-c5cc6ccccc6c6ccccc56)cc4)c4ccc(-c5cccc6c5sc5ccccc56)cc4)cccc23)cc1]

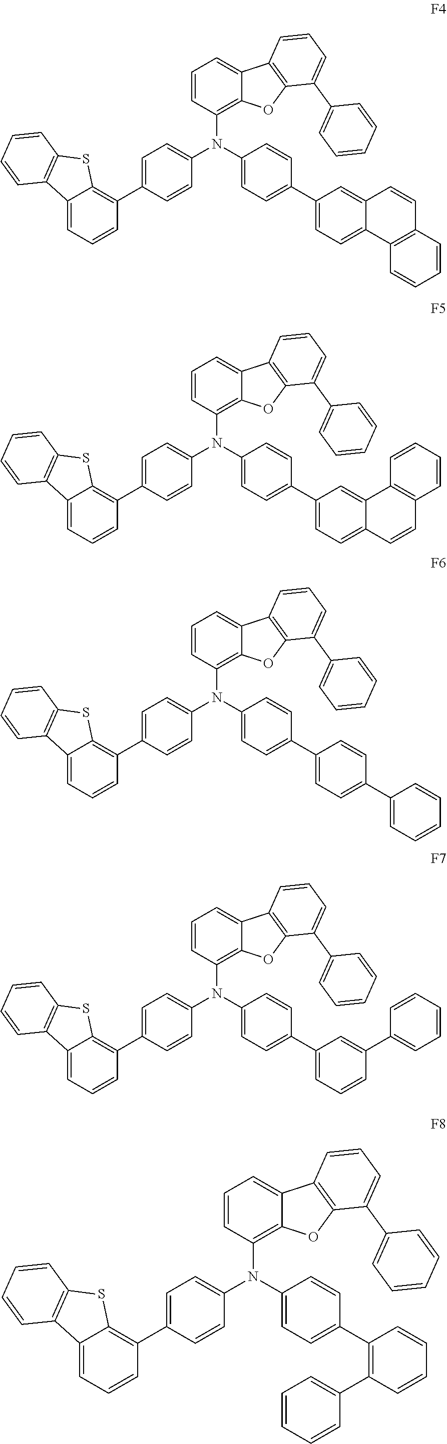 [SMILES: c1ccc(-c2cccc3c2oc2c(N(c4ccc(-c5ccc6c(ccc7ccccc76)c5)cc4)c4ccc(-c5cccc6c5sc5ccccc56)cc4)cccc23)cc1 ; c1ccc(-c2cccc3c2oc2c(N(c4ccc(-c5ccc6ccc7ccccc7c6c5)cc4)c4ccc(-c5cccc6c5sc5ccccc56)cc4)cccc23)cc1 ; c1ccc(-c2ccc(-c3ccc(N(c4ccc(-c5cccc6c5sc5ccccc56)cc4)c4cccc5c4oc4c(-c6ccccc6)cccc45)cc3)cc2)cc1 ; c1ccc(-c2cccc(-c3ccc(N(c4ccc(-c5cccc6c5sc5ccccc56)cc4)c4cccc5c4oc4c(-c6ccccc6)cccc45)cc3)c2)cc1 ; c1ccc(-c2ccccc2-c2ccc(N(c3ccc(-c4cccc5c4sc4ccccc45)cc3)c3cccc4c3oc3c(-c5ccccc5)cccc34)cc2)cc1]

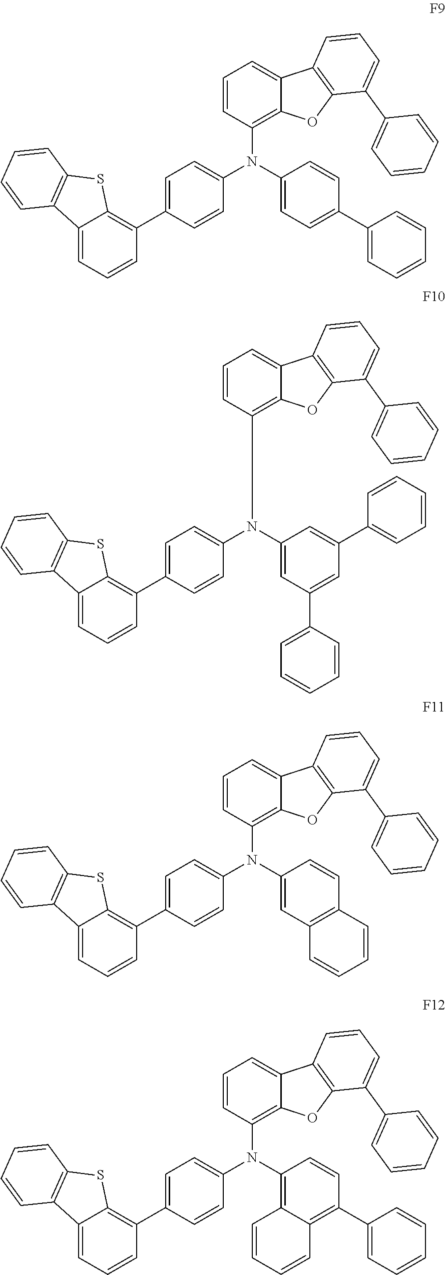

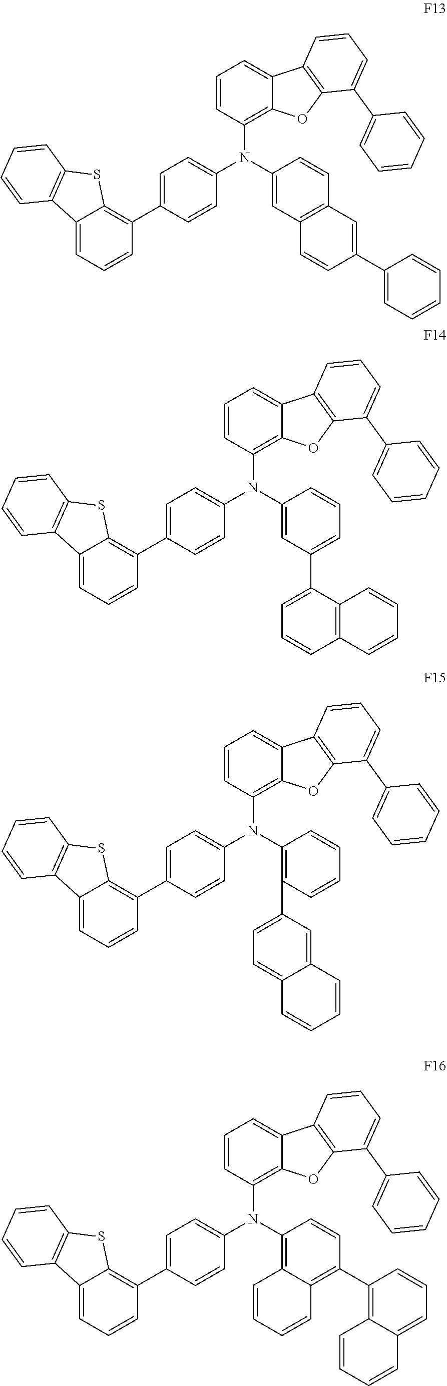 [SMILES: c1ccc(-c2ccc3cc(N(c4ccc(-c5cccc6c5sc5ccccc56)cc4)c4cccc5c4oc4c(-c6ccccc6)cccc45)ccc3c2)cc1 ; c1ccc(-c2cccc3c2oc2c(N(c4ccc(-c5cccc6c5sc5ccccc56)cc4)c4cccc(-c5cccc6ccccc56)c4)cccc23)cc1 ; c1ccc(-c2cccc3c2oc2c(N(c4ccc(-c5cccc6c5sc5ccccc56)cc4)c4ccccc4-c4ccc5ccccc5c4)cccc23)cc1 ; c1ccc(-c2cccc3c2oc2c(N(c4ccc(-c5cccc6c5sc5ccccc56)cc4)c4ccc(-c5cccc6ccccc56)c5ccccc45)cccc23)cc1]

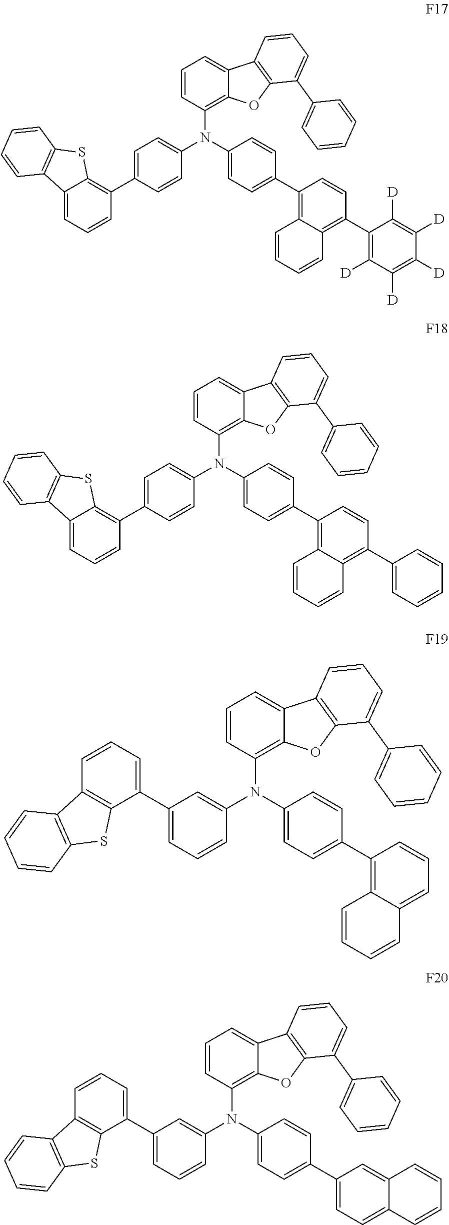 [SMILES: [2H]c1c([2H])c([2H])c(-c2ccc(-c3ccc(N(c4ccc(-c5cccc6c5sc5ccccc56)cc4)c4cccc5c4oc4c(-c6ccccc6)cccc45)cc3)c3ccccc23)c([2H])c1[2H] ; c1ccc(-c2ccc(-c3ccc(N(c4ccc(-c5cccc6c5sc5ccccc56)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5cccc6c5sc5ccccc56)c4)cccc23)cc1 ; c1ccc(-c2cccc3c2oc2c(N(c4ccc(-c5ccc6ccccc6c5)cc4)c4cccc(-c5cccc6c5sc5ccccc56)c4)cccc23)cc1]

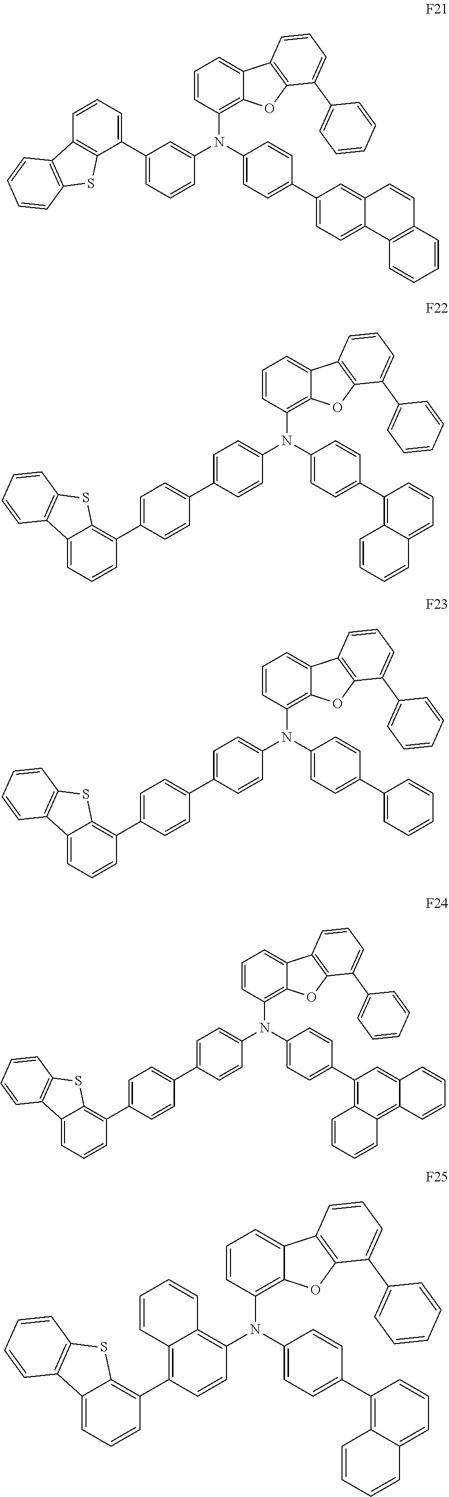

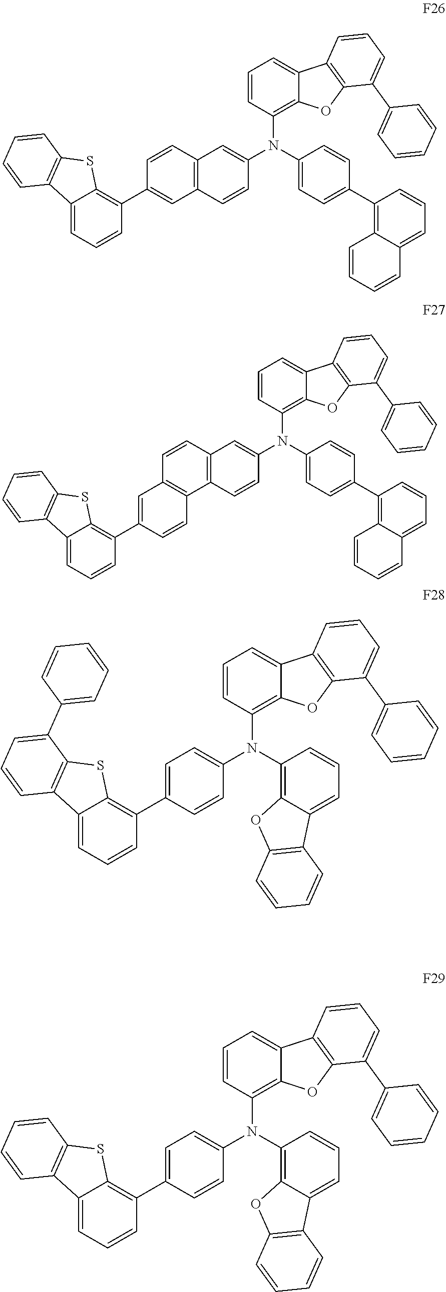 [SMILES: c1ccc(-c2cccc3c2oc2c(N(c4ccc(-c5cccc6ccccc56)cc4)c4ccc5cc(-c6cccc7c6sc6ccccc67)ccc5c4)cccc23)cc1 ; c1ccc(-c2cccc3c2oc2c(N(c4ccc(-c5cccc6ccccc56)cc4)c4ccc5c(ccc6cc(-c7cccc8c7sc7ccccc78)ccc65)c4)cccc23)cc1 ; c1ccc(-c2cccc3c2oc2c(N(c4ccc(-c5cccc6c5sc5c(-c7ccccc7)cccc56)cc4)c4cccc5c4oc4ccccc45)cccc23)cc1 ; c1ccc(-c2cccc3c2oc2c(N(c4ccc(-c5cccc6c5sc5ccccc56)cc4)c4cccc5c4oc4ccccc45)cccc23)cc1]

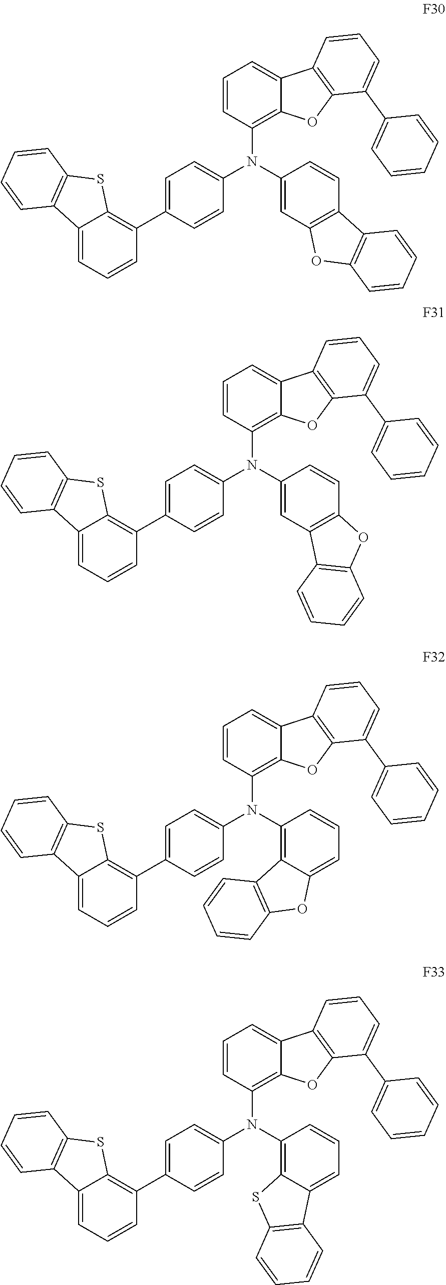 [SMILES: c1ccc(-c2cccc3c2oc2c(N(c4ccc(-c5cccc6c5sc5ccccc56)cc4)c4ccc5c(c4)oc4ccccc45)cccc23)cc1 ; c1ccc(-c2cccc3c2oc2c(N(c4ccc(-c5cccc6c5sc5ccccc56)cc4)c4ccc5oc6ccccc6c5c4)cccc23)cc1 ; c1ccc(-c2cccc3c2oc2c(N(c4ccc(-c5cccc6c5sc5ccccc56)cc4)c4cccc5oc6ccccc6c45)cccc23)cc1 ; c1ccc(-c2cccc3c2oc2c(N(c4ccc(-c5cccc6c5sc5ccccc56)cc4)c4cccc5c4sc4ccccc45)cccc23)cc1]

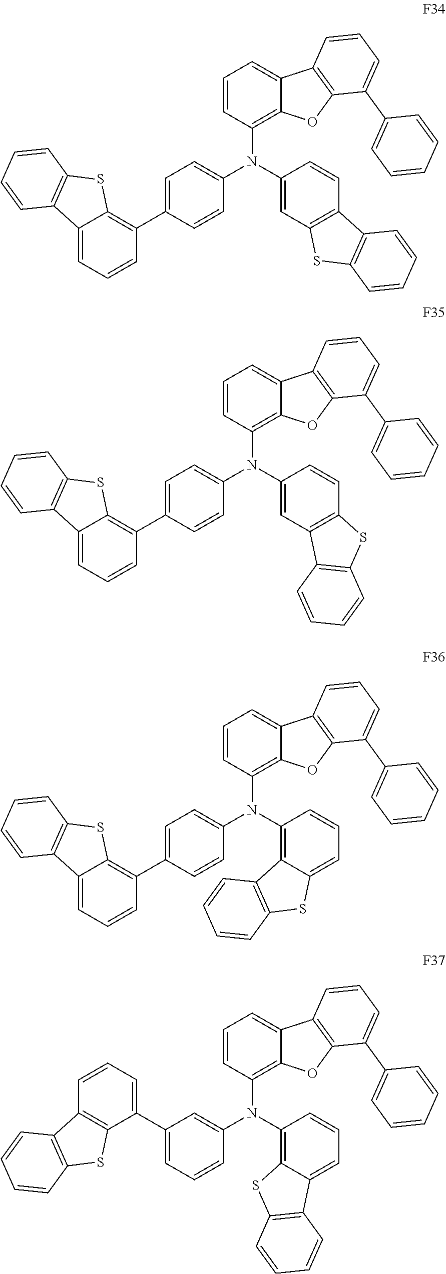 [SMILES: c1ccc(-c2cccc3c2oc2c(N(c4ccc(-c5cccc6c5sc5ccccc56)cc4)c4ccc5c(c4)sc4ccccc45)cccc23)cc1 ; c1ccc(-c2cccc3c2oc2c(N(c4ccc(-c5cccc6c5sc5ccccc56)cc4)c4ccc5sc6ccccc6c5c4)cccc23)cc1 ; c1ccc(-c2cccc3c2oc2c(N(c4ccc(-c5cccc6c5sc5ccccc56)cc4)c4cccc5sc6ccccc6c45)cccc23)cc1 ; c1ccc(-c2cccc3c2oc2c(N(c4cccc(-c5cccc6c5sc5ccccc56)c4)c4cccc5c4sc4ccccc45)cccc23)cc1]

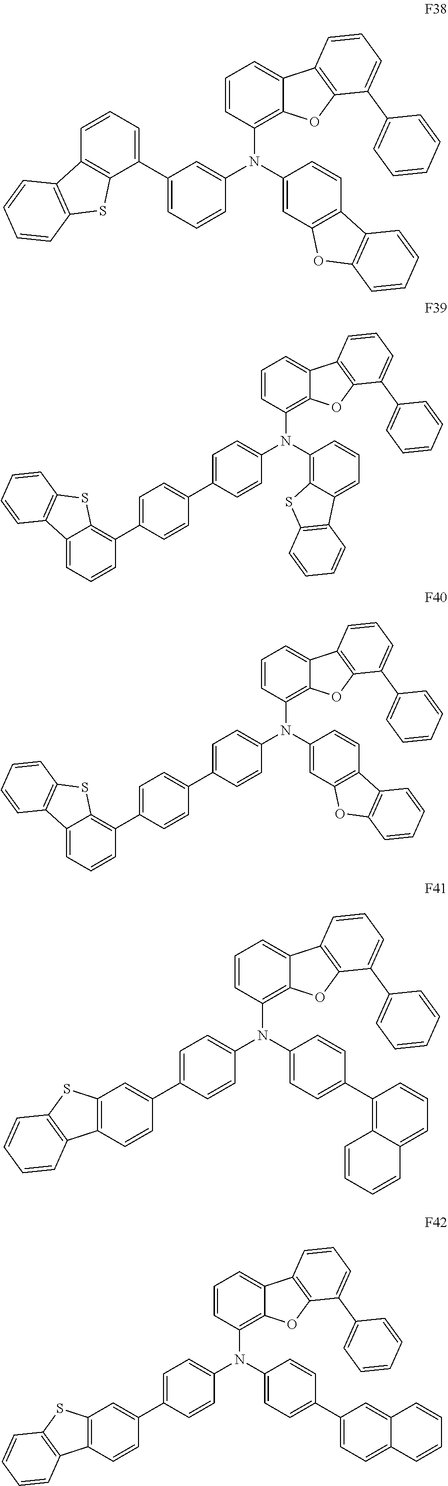 [SMILES: c1ccc(-c2cccc3c2oc2c(N(c4cccc(-c5cccc6c5sc5ccccc56)c4)c4ccc5c(c4)oc4ccccc45)cccc23)cc1 ; c1ccc(-c2cccc3c2oc2c(N(c4ccc(-c5ccc(-c6cccc7c6sc6ccccc67)cc5)cc4)c4cccc5c4sc4ccccc45)cccc23)cc1 ; c1ccc(-c2cccc3c2oc2c(N(c4ccc(-c5ccc(-c6cccc7c6sc6ccccc67)cc5)cc4)c4ccc5c(c4)oc4ccccc45)cccc23)cc1 ; c1ccc(-c2cccc3c2oc2c(N(c4ccc(-c5ccc6c(c5)sc5ccccc56)cc4)c4ccc(-c5cccc6ccccc56)cc4)cccc23)cc1 ; c1ccc(-c2cccc3c2oc2c(N(c4ccc(-c5ccc6ccccc6c5)cc4)c4ccc(-c5ccc6c(c5)sc5ccccc56)cc4)cccc23)cc1]

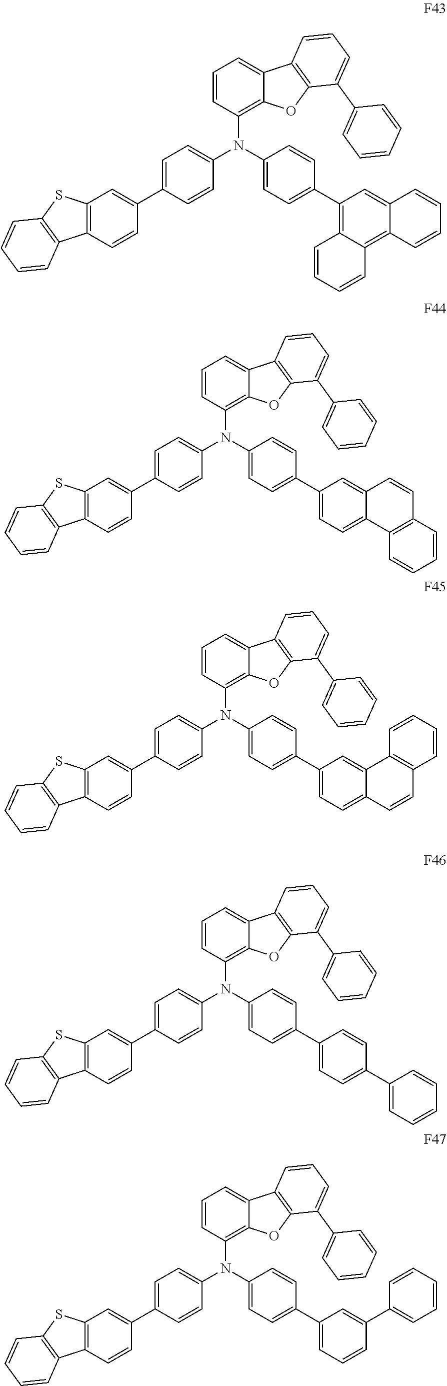

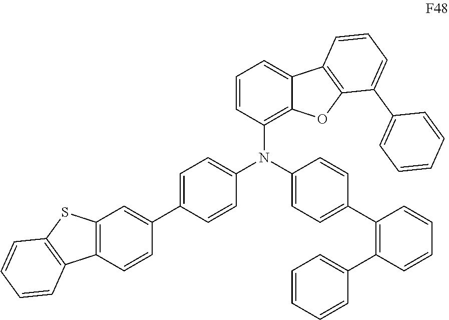 [SMILES: c1ccc(-c2ccccc2-c2ccc(N(c3ccc(-c4ccc5c(c4)sc4ccccc45)cc3)c3cccc4c3oc3c(-c5ccccc5)cccc34)cc2)cc1]

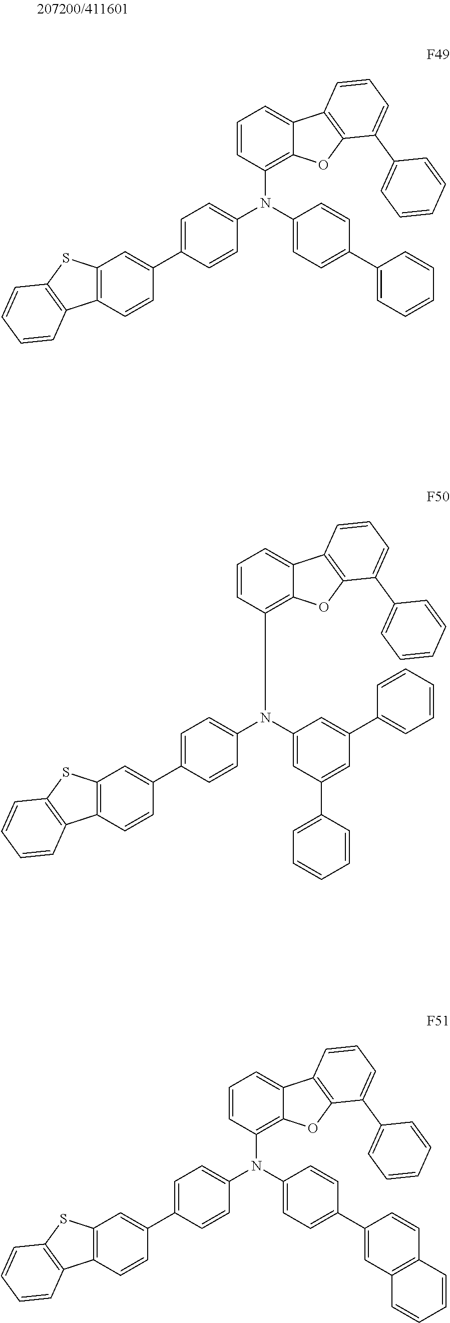 [SMILES: c1ccc(-c2ccc(N(c3ccc(-c4ccc5c(c4)sc4ccccc45)cc3)c3cccc4c3oc3c(-c5ccccc5)cccc34)cc2)cc1 ; c1ccc(-c2cc(-c3ccccc3)cc(N(c3ccc(-c4ccc5c(c4)sc4ccccc45)cc3)c3cccc4c3oc3c(-c5ccccc5)cccc34)c2)cc1 ; c1ccc(-c2cccc3c2oc2c(N(c4ccc(-c5ccc6ccccc6c5)cc4)c4ccc(-c5ccc6c(c5)sc5ccccc56)cc4)cccc23)cc1]

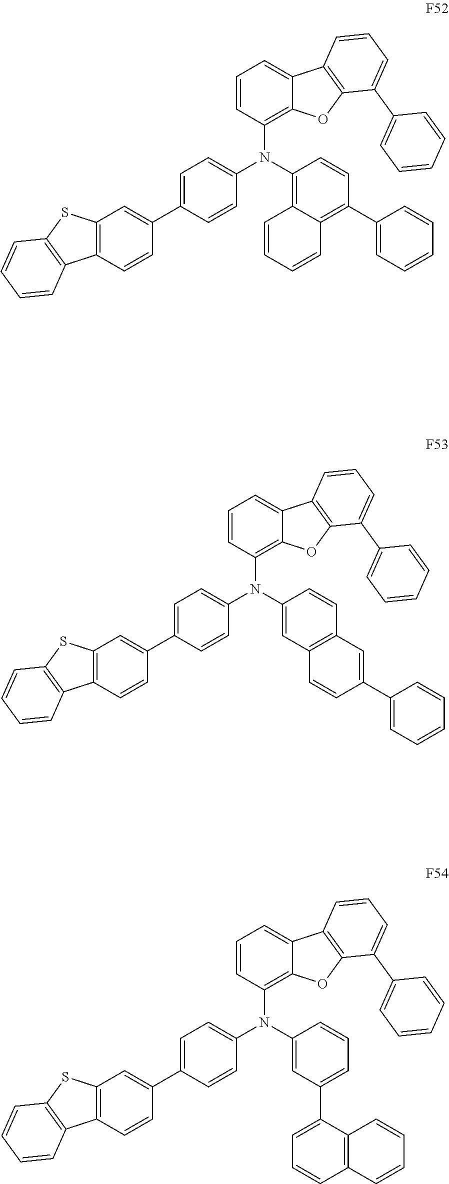 [SMILES: c1ccc(-c2ccc(N(c3ccc(-c4ccc5c(c4)sc4ccccc45)cc3)c3cccc4c3oc3c(-c5ccccc5)cccc34)c3ccccc23)cc1 ; c1ccc(-c2ccc3cc(N(c4ccc(-c5ccc6c(c5)sc5ccccc56)cc4)c4cccc5c4oc4c(-c6ccccc6)cccc45)ccc3c2)cc1 ; c1ccc(-c2cccc3c2oc2c(N(c4ccc(-c5ccc6c(c5)sc5ccccc56)cc4)c4cccc(-c5cccc6ccccc56)c4)cccc23)cc1]

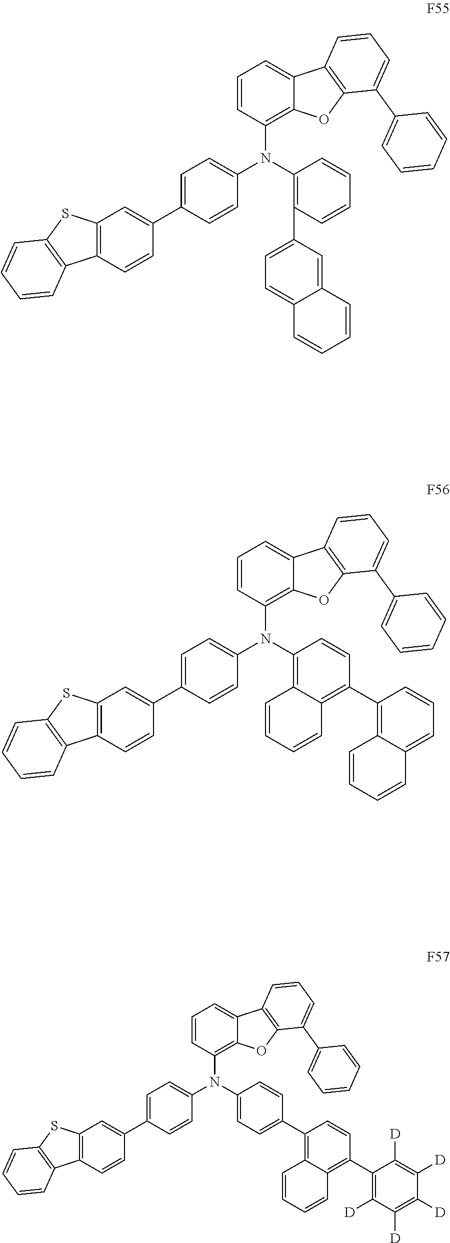 [SMILES: c1ccc(-c2cccc3c2oc2c(N(c4ccc(-c5ccc6c(c5)sc5ccccc56)cc4)c4ccccc4-c4ccc5ccccc5c4)cccc23)cc1 ; c1ccc(-c2cccc3c2oc2c(N(c4ccc(-c5ccc6c(c5)sc5ccccc56)cc4)c4ccc(-c5cccc6ccccc56)c5ccccc45)cccc23)cc1 ; [2H]c1c([2H])c([2H])c(-c2ccc(-c3ccc(N(c4ccc(-c5ccc6c(c5)sc5ccccc56)cc4)c4cccc5c4oc4c(-c6ccccc6)cccc45)cc3)c3ccccc23)c([2H])c1[2H]]

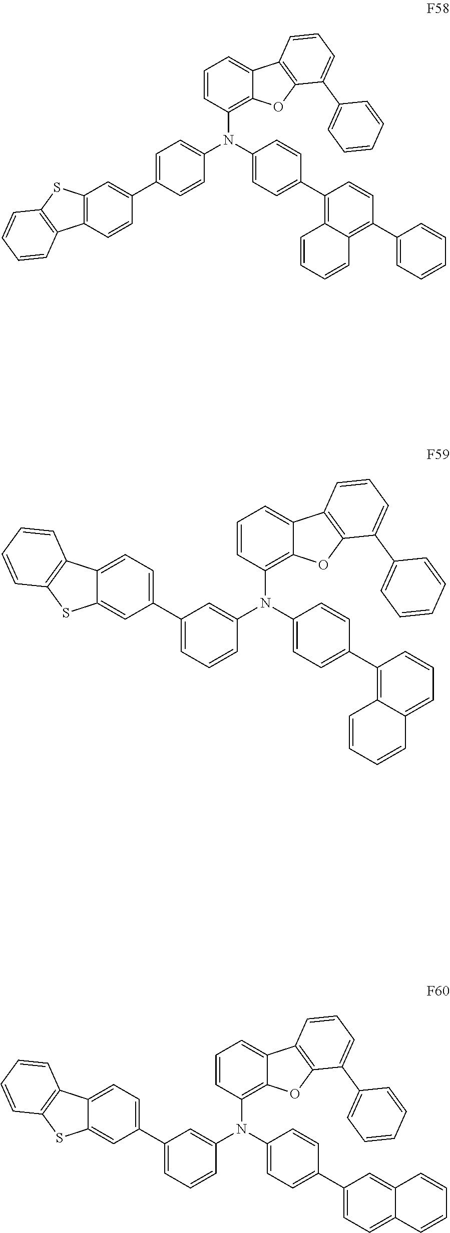 [SMILES: c1ccc(-c2ccc(-c3ccc(N(c4ccc(-c5ccc6c(c5)sc5ccccc56)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5ccc6c(c5)sc5ccccc56)c4)cccc23)cc1 ; c1ccc(-c2cccc3c2oc2c(N(c4ccc(-c5ccc6ccccc6c5)cc4)c4cccc(-c5ccc6c(c5)sc5ccccc56)c4)cccc23)cc1]

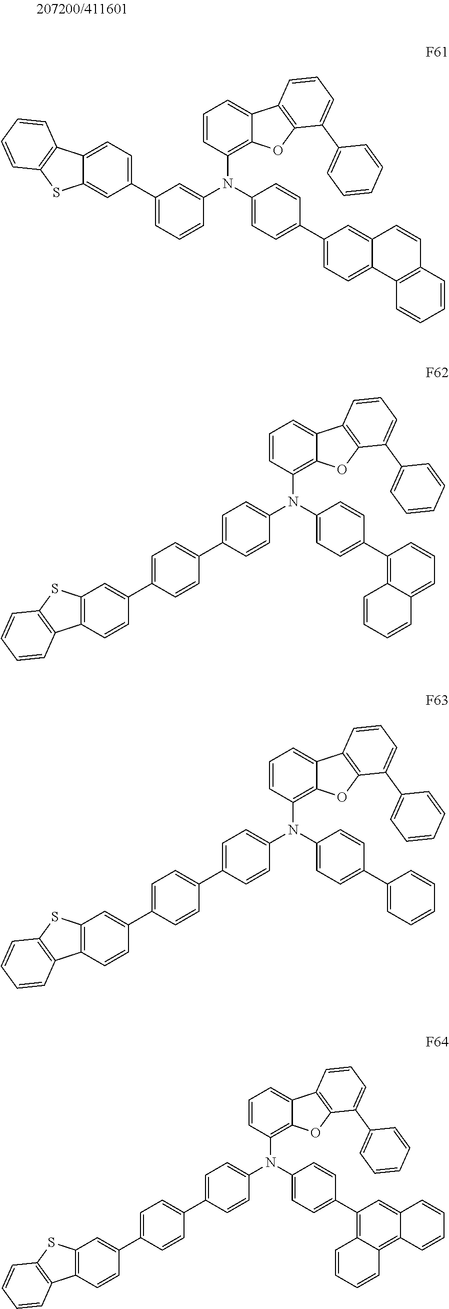 [SMILES: c1ccc(-c2cccc3c2oc2c(N(c4ccc(-c5ccc6c(ccc7ccccc76)c5)cc4)c4cccc(-c5ccc6c(c5)sc5ccccc56)c4)cccc23)cc1 ; c1ccc(-c2cccc3c2oc2c(N(c4ccc(-c5ccc(-c6ccc7c(c6)sc6ccccc67)cc5)cc4)c4ccc(-c5cccc6ccccc56)cc4)cccc23)cc1 ; c1ccc(-c2ccc(N(c3ccc(-c4ccc(-c5ccc6c(c5)sc5ccccc56)cc4)cc3)c3cccc4c3oc3c(-c5ccccc5)cccc34)cc2)cc1 ; c1ccc(-c2cccc3c2oc2c(N(c4ccc(-c5ccc(-c6ccc7c(c6)sc6ccccc67)cc5)cc4)c4ccc(-c5cc6ccccc6c6ccccc56)cc4)cccc23)cc1]

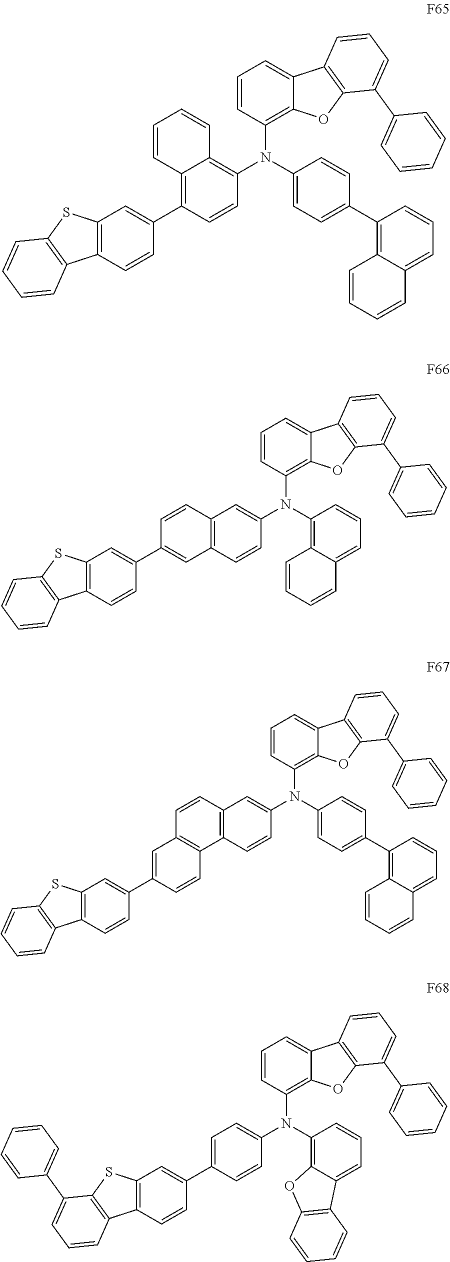 [SMILES: c1ccc(-c2cccc3c2oc2c(N(c4ccc(-c5cccc6ccccc56)cc4)c4ccc(-c5ccc6c(c5)sc5ccccc56)c5ccccc45)cccc23)cc1 ; c1ccc(-c2cccc3c2oc2c(N(c4ccc5cc(-c6ccc7c(c6)sc6ccccc67)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7ccc8c(c7)sc7ccccc78)ccc65)c4)cccc23)cc1 ; c1ccc(-c2cccc3c2oc2c(N(c4ccc(-c5ccc6c(c5)sc5c(-c7ccccc7)cccc56)cc4)c4cccc5c4oc4ccccc45)cccc23)cc1]

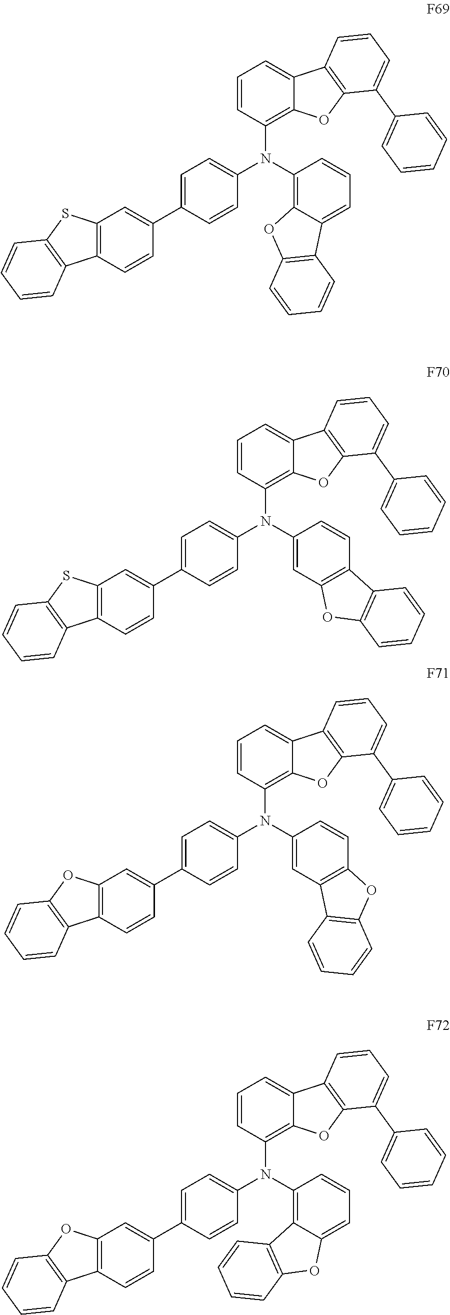 [SMILES: c1ccc(-c2cccc3c2oc2c(N(c4ccc(-c5ccc6c(c5)sc5ccccc56)cc4)c4cccc5c4oc4ccccc45)cccc23)cc1 ; c1ccc(-c2cccc3c2oc2c(N(c4ccc(-c5ccc6c(c5)sc5ccccc56)cc4)c4ccc5c(c4)oc4ccccc45)cccc23)cc1 ; c1ccc(-c2cccc3c2oc2c(N(c4ccc(-c5ccc6c(c5)oc5ccccc56)cc4)c4ccc5oc6ccccc6c5c4)cccc23)cc1 ; c1ccc(-c2cccc3c2oc2c(N(c4ccc(-c5ccc6c(c5)oc5ccccc56)cc4)c4cccc5oc6ccccc6c45)cccc23)cc1]

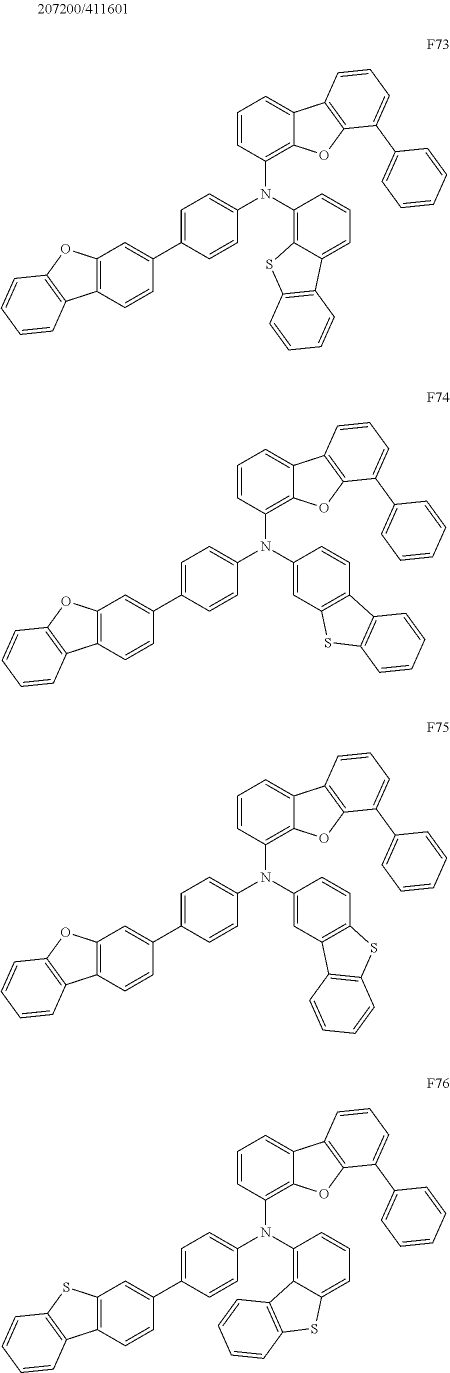 [SMILES: c1ccc(-c2cccc3c2oc2c(N(c4ccc(-c5ccc6c(c5)oc5ccccc56)cc4)c4cccc5c4sc4ccccc45)cccc23)cc1 ; c1ccc(-c2cccc3c2oc2c(N(c4ccc(-c5ccc6c(c5)oc5ccccc56)cc4)c4ccc5c(c4)sc4ccccc45)cccc23)cc1 ; c1ccc(-c2cccc3c2oc2c(N(c4ccc(-c5ccc6c(c5)oc5ccccc56)cc4)c4ccc5sc6ccccc6c5c4)cccc23)cc1 ; c1ccc(-c2cccc3c2oc2c(N(c4ccc(-c5ccc6c(c5)sc5ccccc56)cc4)c4cccc5sc6ccccc6c45)cccc23)cc1]

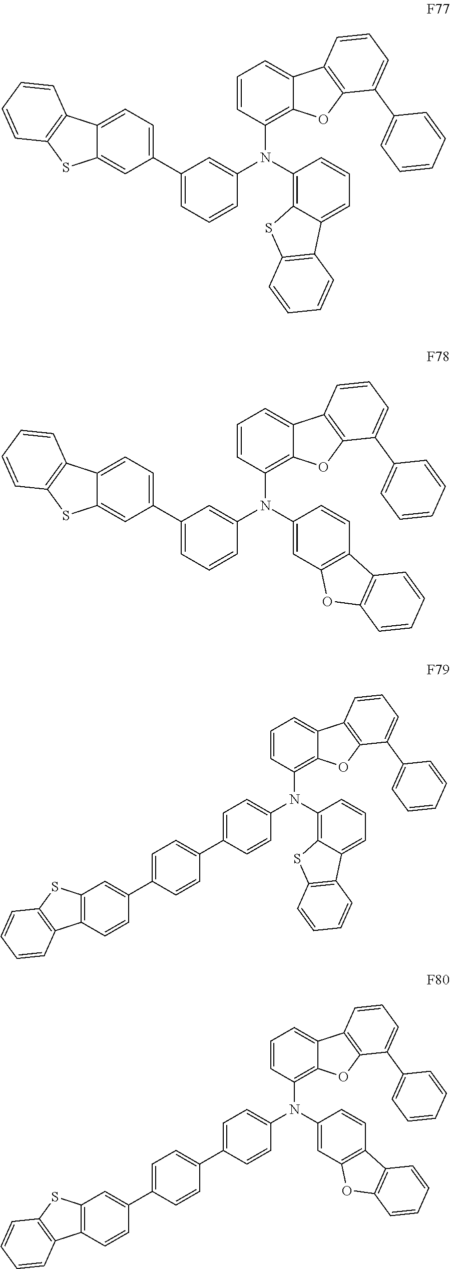 [SMILES: c1ccc(-c2cccc3c2oc2c(N(c4cccc(-c5ccc6c(c5)sc5ccccc56)c4)c4cccc5c4sc4ccccc45)cccc23)cc1 ; c1ccc(-c2cccc3c2oc2c(N(c4cccc(-c5ccc6c(c5)sc5ccccc56)c4)c4ccc5c(c4)oc4ccccc45)cccc23)cc1 ; c1ccc(-c2cccc3c2oc2c(N(c4ccc(-c5ccc(-c6ccc7c(c6)sc6ccccc67)cc5)cc4)c4cccc5c4sc4ccccc45)cccc23)cc1 ; c1ccc(-c2cccc3c2oc2c(N(c4ccc(-c5ccc(-c6ccc7c(c6)sc6ccccc67)cc5)cc4)c4ccc5c(c4)oc4ccccc45)cccc23)cc1]

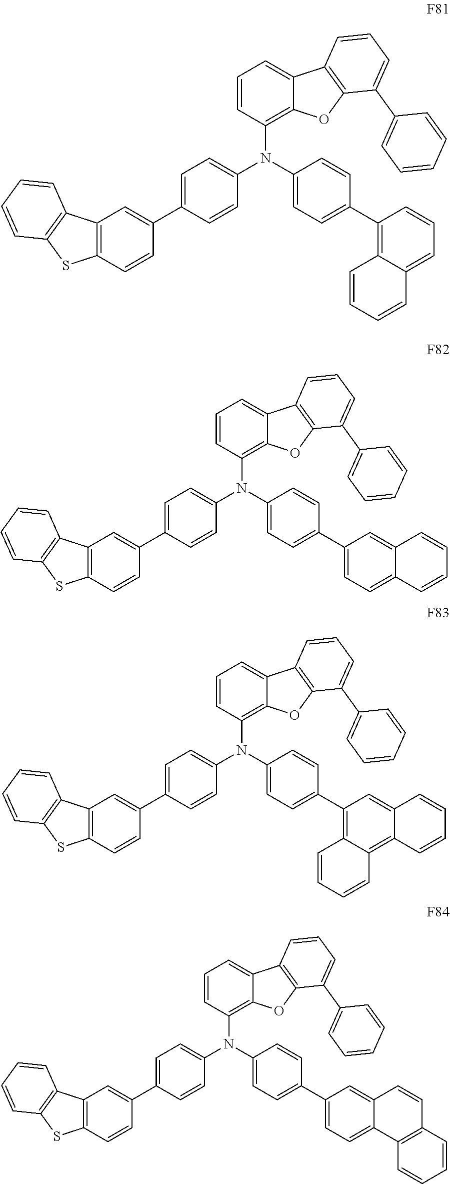 [SMILES: c1ccc(-c2cccc3c2oc2c(N(c4ccc(-c5ccc6sc7ccccc7c6c5)cc4)c4ccc(-c5cccc6ccccc56)cc4)cccc23)cc1 ; c1ccc(-c2cccc3c2oc2c(N(c4ccc(-c5ccc6ccccc6c5)cc4)c4ccc(-c5ccc6sc7ccccc7c6c5)cc4)cccc23)cc1 ; c1ccc(-c2cccc3c2oc2c(N(c4ccc(-c5ccc6sc7ccccc7c6c5)cc4)c4ccc(-c5cc6ccccc6c6ccccc56)cc4)cccc23)cc1 ; c1ccc(-c2cccc3c2oc2c(N(c4ccc(-c5ccc6c(ccc7ccccc76)c5)cc4)c4ccc(-c5ccc6sc7ccccc7c6c5)cc4)cccc23)cc1]

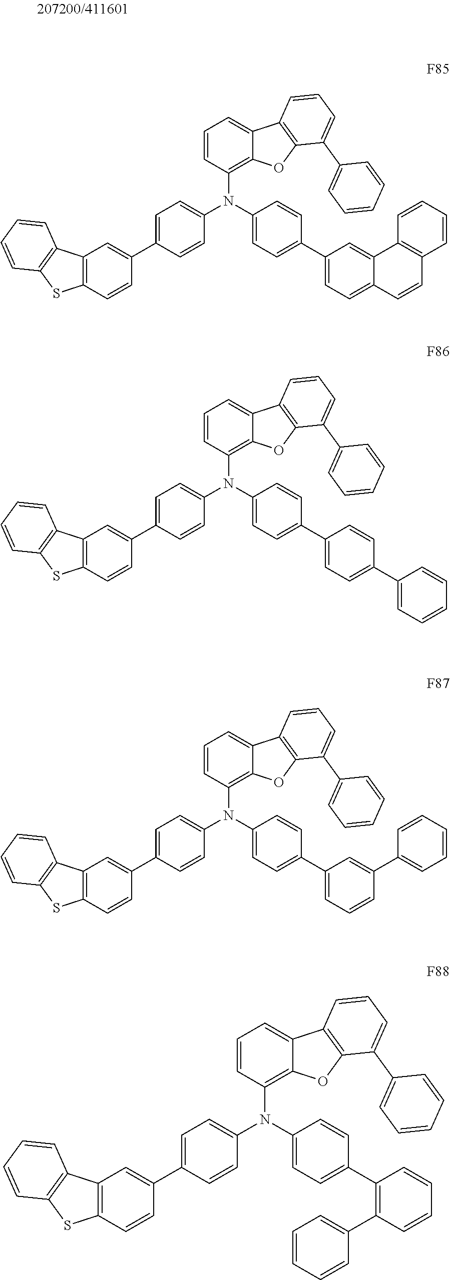 [SMILES: c1ccc(-c2cccc3c2oc2c(N(c4ccc(-c5ccc6ccc7ccccc7c6c5)cc4)c4ccc(-c5ccc6sc7ccccc7c6c5)cc4)cccc23)cc1 ; c1ccc(-c2ccc(-c3ccc(N(c4ccc(-c5ccc6sc7ccccc7c6c5)cc4)c4cccc5c4oc4c(-c6ccccc6)cccc45)cc3)cc2)cc1 ; c1ccc(-c2cccc(-c3ccc(N(c4ccc(-c5ccc6sc7ccccc7c6c5)cc4)c4cccc5c4oc4c(-c6ccccc6)cccc45)cc3)c2)cc1 ; c1ccc(-c2ccccc2-c2ccc(N(c3ccc(-c4ccc5sc6ccccc6c5c4)cc3)c3cccc4c3oc3c(-c5ccccc5)cccc34)cc2)cc1]

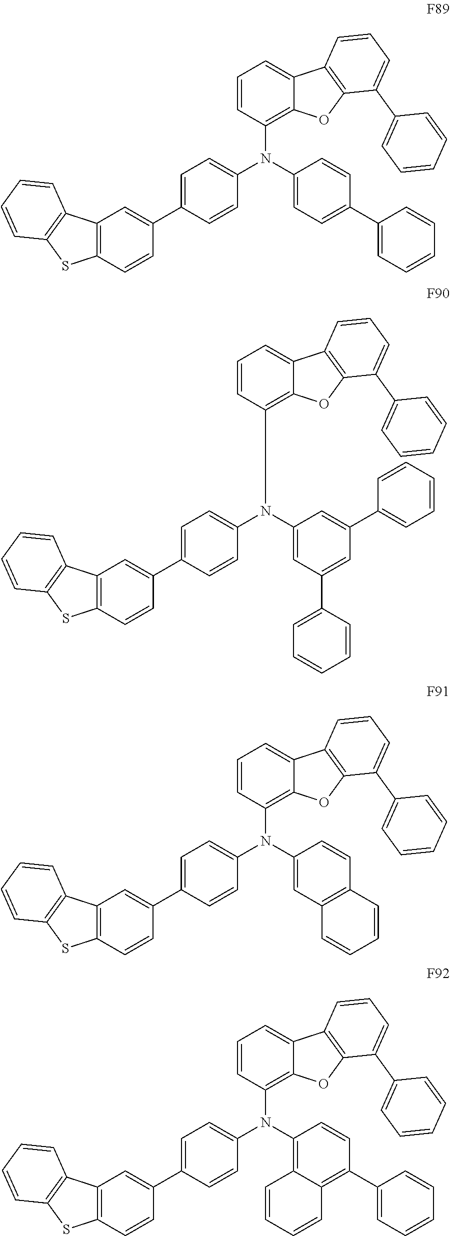 [SMILES: c1ccc(-c2ccc(N(c3ccc(-c4ccc5sc6ccccc6c5c4)cc3)c3cccc4c3oc3c(-c5ccccc5)cccc34)cc2)cc1 ; c1ccc(-c2cc(-c3ccccc3)cc(N(c3ccc(-c4ccc5sc6ccccc6c5c4)cc3)c3cccc4c3oc3c(-c5ccccc5)cccc34)c2)cc1 ; c1ccc(-c2cccc3c2oc2c(N(c4ccc(-c5ccc6sc7ccccc7c6c5)cc4)c4ccc5ccccc5c4)cccc23)cc1 ; c1ccc(-c2ccc(N(c3ccc(-c4ccc5sc6ccccc6c5c4)cc3)c3cccc4c3oc3c(-c5ccccc5)cccc34)c3ccccc23)cc1]

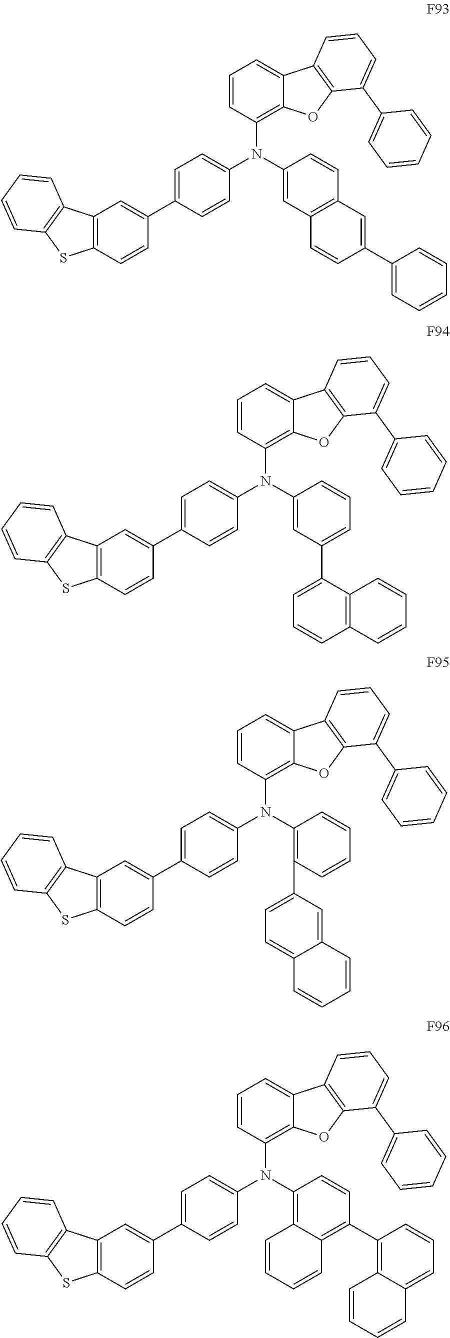

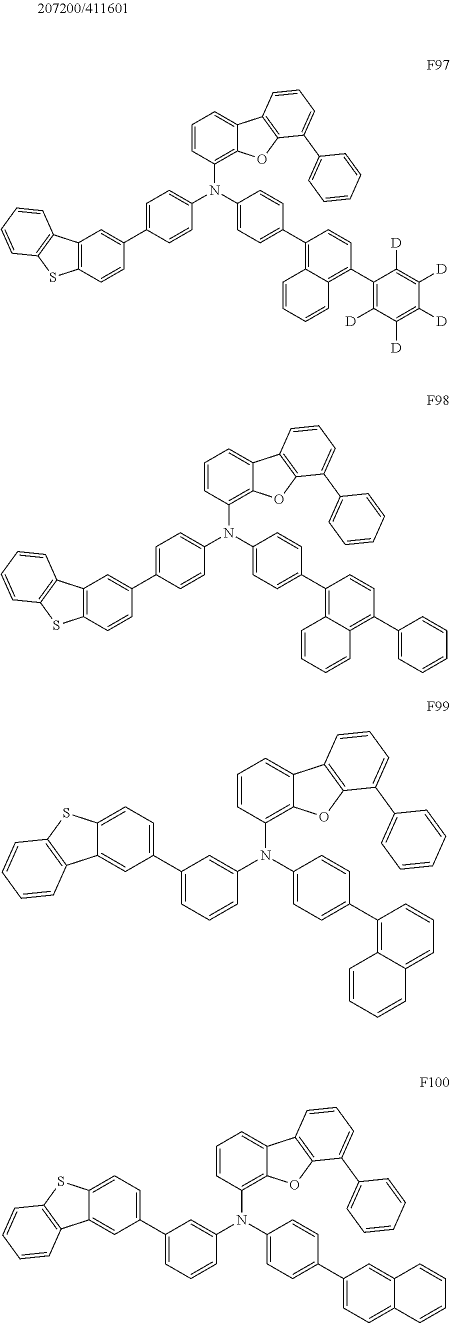 [SMILES: [2H]c1c([2H])c([2H])c(-c2ccc(-c3ccc(N(c4ccc(-c5ccc6sc7ccccc7c6c5)cc4)c4cccc5c4oc4c(-c6ccccc6)cccc45)cc3)c3ccccc23)c([2H])c1[2H] ; c1ccc(-c2ccc(-c3ccc(N(c4ccc(-c5ccc6sc7ccccc7c6c5)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5ccc6sc7ccccc7c6c5)c4)cccc23)cc1 ; c1ccc(-c2cccc3c2oc2c(N(c4ccc(-c5ccc6ccccc6c5)cc4)c4cccc(-c5ccc6sc7ccccc7c6c5)c4)cccc23)cc1]

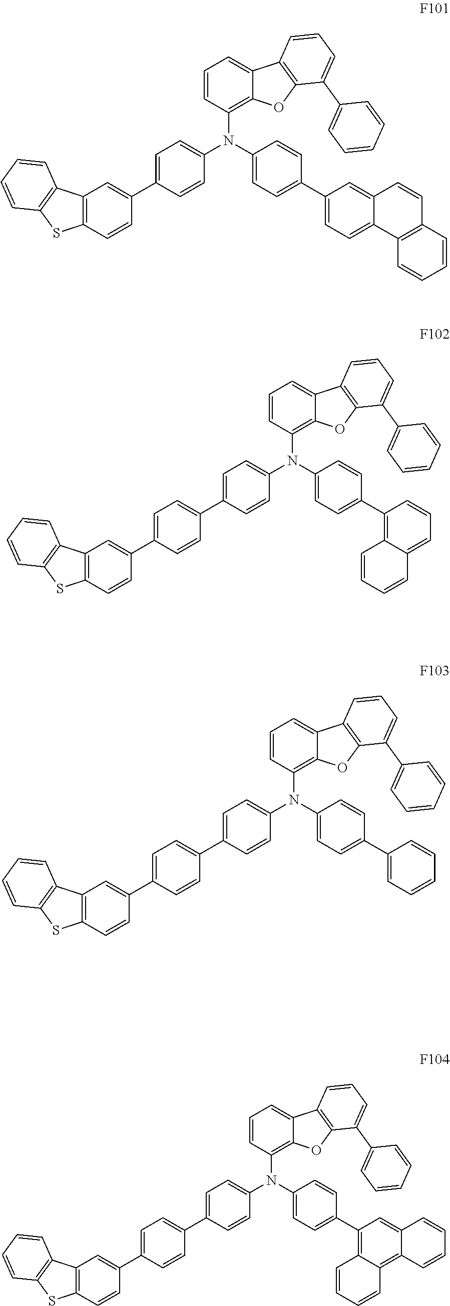 [SMILES: c1ccc(-c2cccc3c2oc2c(N(c4ccc(-c5ccc6c(ccc7ccccc76)c5)cc4)c4ccc(-c5ccc6sc7ccccc7c6c5)cc4)cccc23)cc1 ; c1ccc(-c2cccc3c2oc2c(N(c4ccc(-c5ccc(-c6ccc7sc8ccccc8c7c6)cc5)cc4)c4ccc(-c5cccc6ccccc56)cc4)cccc23)cc1 ; c1ccc(-c2ccc(N(c3ccc(-c4ccc(-c5ccc6sc7ccccc7c6c5)cc4)cc3)c3cccc4c3oc3c(-c5ccccc5)cccc34)cc2)cc1 ; c1ccc(-c2cccc3c2oc2c(N(c4ccc(-c5ccc(-c6ccc7sc8ccccc8c7c6)cc5)cc4)c4ccc(-c5cc6ccccc6c6ccccc56)cc4)cccc23)cc1]

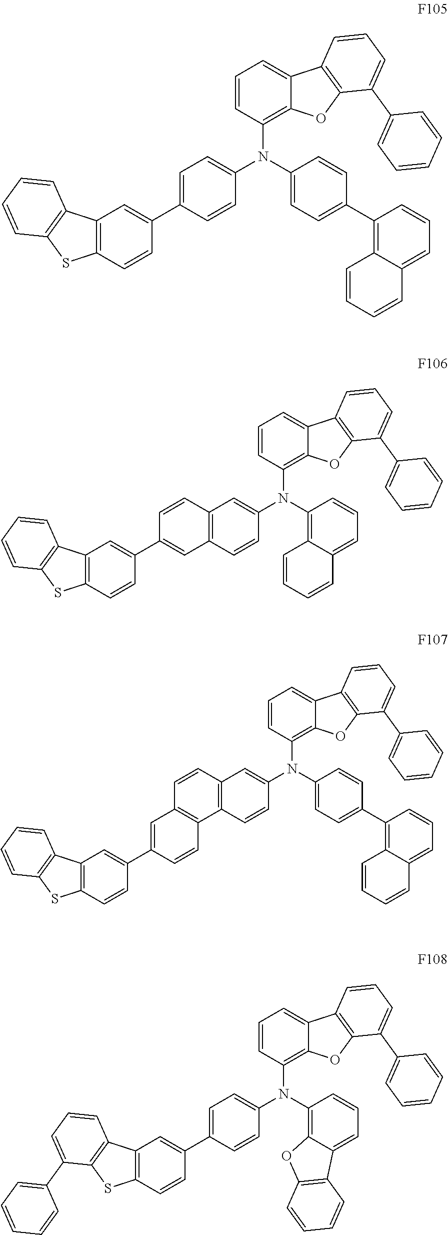 [SMILES: c1ccc(-c2cccc3c2oc2c(N(c4ccc(-c5ccc6sc7ccccc7c6c5)cc4)c4ccc(-c5cccc6ccccc56)cc4)cccc23)cc1 ; c1ccc(-c2cccc3c2oc2c(N(c4ccc5cc(-c6ccc7sc8ccccc8c7c6)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7ccc8sc9ccccc9c8c7)ccc65)c4)cccc23)cc1 ; c1ccc(-c2cccc3c2oc2c(N(c4ccc(-c5ccc6sc7c(-c8ccccc8)cccc7c6c5)cc4)c4cccc5c4oc4ccccc45)cccc23)cc1]

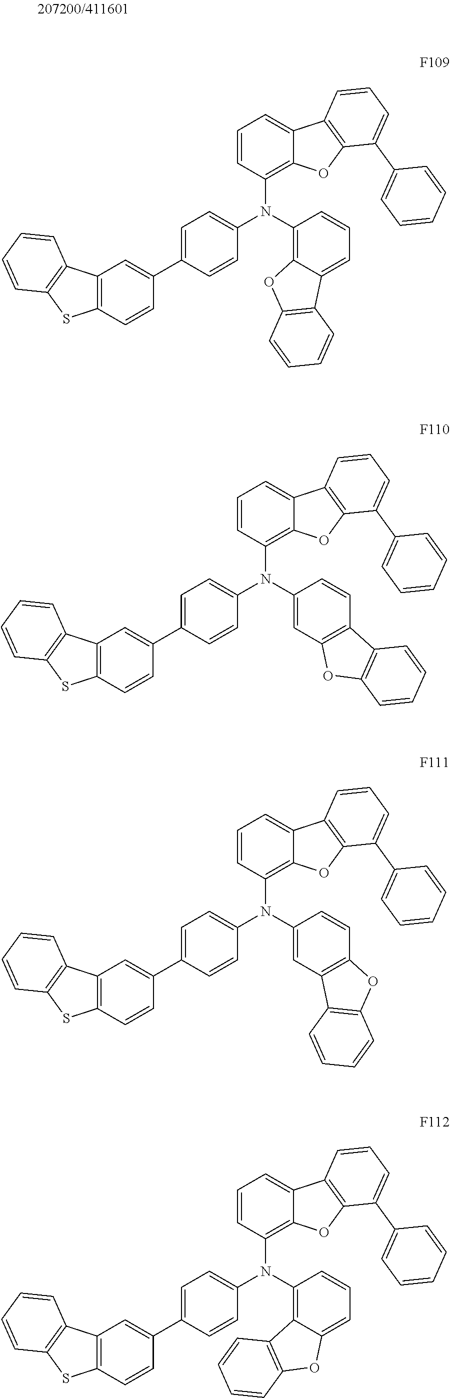 [SMILES: c1ccc(-c2cccc3c2oc2c(N(c4ccc(-c5ccc6sc7ccccc7c6c5)cc4)c4cccc5c4oc4ccccc45)cccc23)cc1 ; c1ccc(-c2cccc3c2oc2c(N(c4ccc(-c5ccc6sc7ccccc7c6c5)cc4)c4ccc5c(c4)oc4ccccc45)cccc23)cc1 ; c1ccc(-c2cccc3c2oc2c(N(c4ccc(-c5ccc6sc7ccccc7c6c5)cc4)c4ccc5oc6ccccc6c5c4)cccc23)cc1 ; c1ccc(-c2cccc3c2oc2c(N(c4ccc(-c5ccc6sc7ccccc7c6c5)cc4)c4cccc5oc6ccccc6c45)cccc23)cc1]

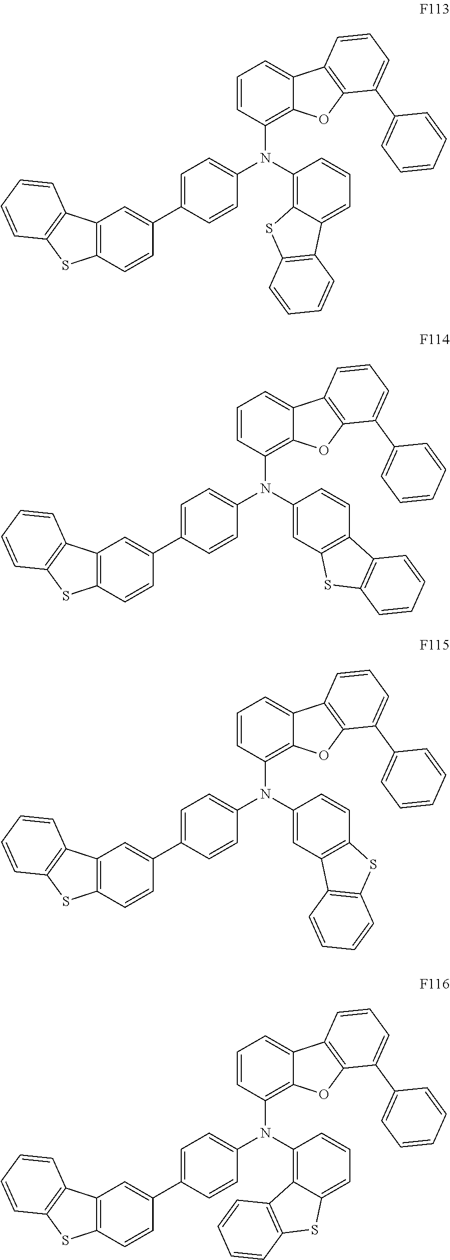 [SMILES: c1ccc(-c2cccc3c2oc2c(N(c4ccc(-c5ccc6sc7ccccc7c6c5)cc4)c4cccc5c4sc4ccccc45)cccc23)cc1 ; c1ccc(-c2cccc3c2oc2c(N(c4ccc(-c5ccc6sc7ccccc7c6c5)cc4)c4ccc5c(c4)sc4ccccc45)cccc23)cc1 ; c1ccc(-c2cccc3c2oc2c(N(c4ccc(-c5ccc6sc7ccccc7c6c5)cc4)c4ccc5sc6ccccc6c5c4)cccc23)cc1 ; c1ccc(-c2cccc3c2oc2c(N(c4ccc(-c5ccc6sc7ccccc7c6c5)cc4)c4cccc5sc6ccccc6c45)cccc23)cc1]

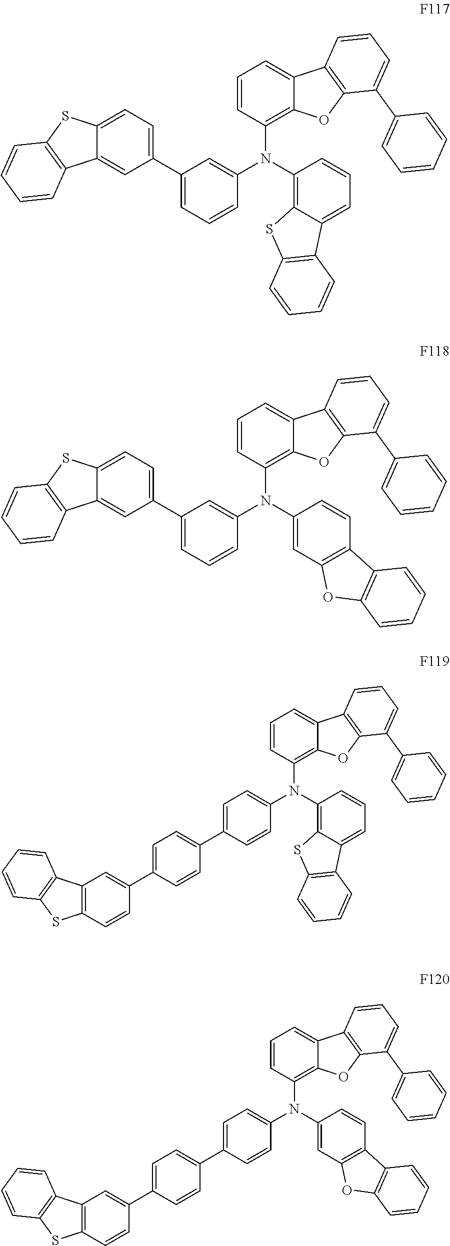 [SMILES: c1ccc(-c2cccc3c2oc2c(N(c4cccc(-c5ccc6sc7ccccc7c6c5)c4)c4cccc5c4sc4ccccc45)cccc23)cc1 ; c1ccc(-c2cccc3c2oc2c(N(c4cccc(-c5ccc6sc7ccccc7c6c5)c4)c4ccc5c(c4)oc4ccccc45)cccc23)cc1 ; c1ccc(-c2cccc3c2oc2c(N(c4ccc(-c5ccc(-c6ccc7sc8ccccc8c7c6)cc5)cc4)c4cccc5c4sc4ccccc45)cccc23)cc1 ; c1ccc(-c2cccc3c2oc2c(N(c4ccc(-c5ccc(-c6ccc7sc8ccccc8c7c6)cc5)cc4)c4ccc5c(c4)oc4ccccc45)cccc23)cc1]

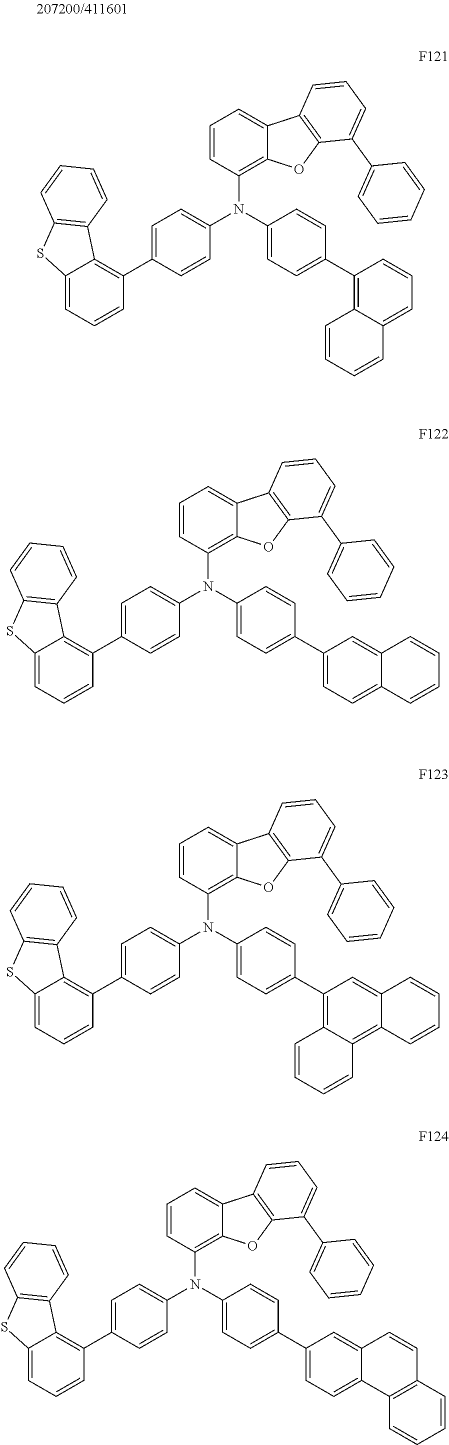 [SMILES: c1ccc(-c2cccc3c2oc2c(N(c4ccc(-c5cccc6ccccc56)cc4)c4ccc(-c5cccc6sc7ccccc7c56)cc4)cccc23)cc1 ; c1ccc(-c2cccc3c2oc2c(N(c4ccc(-c5ccc6ccccc6c5)cc4)c4ccc(-c5cccc6sc7ccccc7c56)cc4)cccc23)cc1 ; c1ccc(-c2cccc3c2oc2c(N(c4ccc(-c5cc6ccccc6c6ccccc56)cc4)c4ccc(-c5cccc6sc7ccccc7c56)cc4)cccc23)cc1 ; c1ccc(-c2cccc3c2oc2c(N(c4ccc(-c5ccc6c(ccc7ccccc76)c5)cc4)c4ccc(-c5cccc6sc7ccccc7c56)cc4)cccc23)cc1]

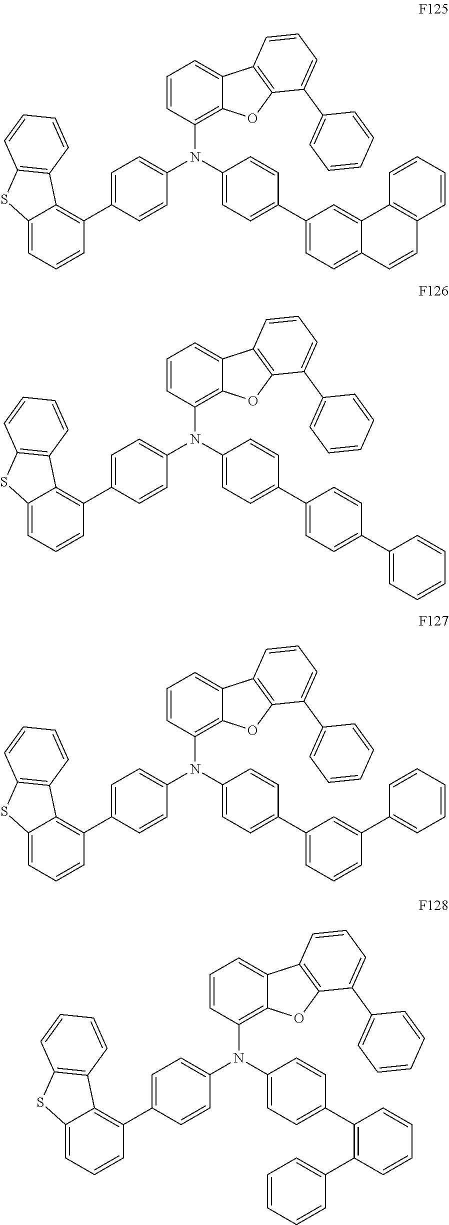 [SMILES: c1ccc(-c2cccc3c2oc2c(N(c4ccc(-c5ccc6ccc7ccccc7c6c5)cc4)c4ccc(-c5cccc6sc7ccccc7c56)cc4)cccc23)cc1 ; c1ccc(-c2ccc(-c3ccc(N(c4ccc(-c5cccc6sc7ccccc7c56)cc4)c4cccc5c4oc4c(-c6ccccc6)cccc45)cc3)cc2)cc1 ; c1ccc(-c2cccc(-c3ccc(N(c4ccc(-c5cccc6sc7ccccc7c56)cc4)c4cccc5c4oc4c(-c6ccccc6)cccc45)cc3)c2)cc1 ; c1ccc(-c2ccccc2-c2ccc(N(c3ccc(-c4cccc5sc6ccccc6c45)cc3)c3cccc4c3oc3c(-c5ccccc5)cccc34)cc2)cc1]

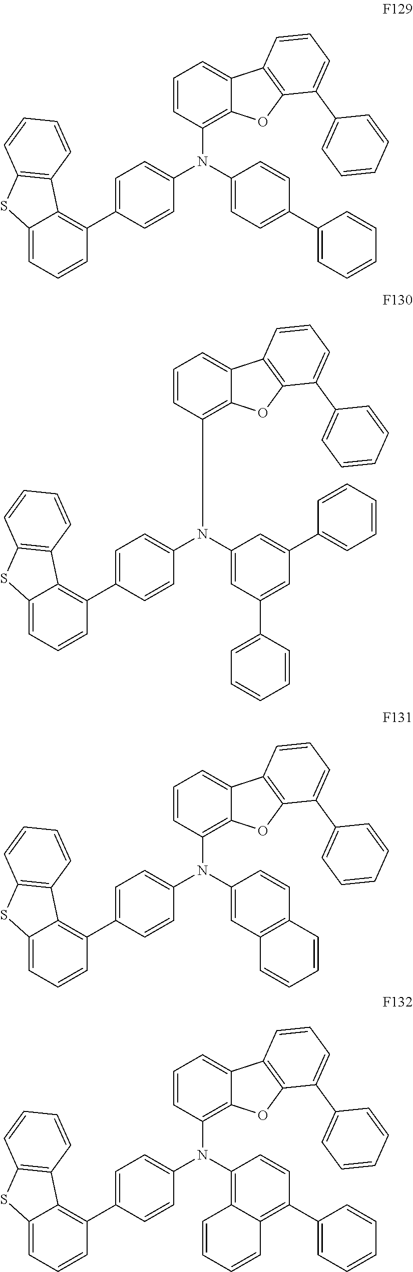

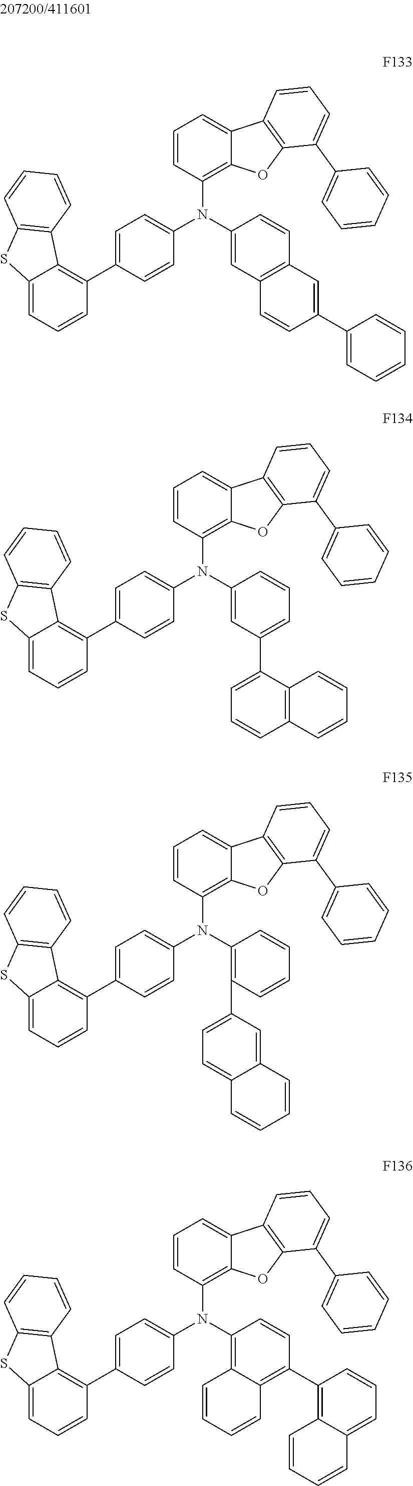 [SMILES: c1ccc(-c2ccc3cc(N(c4ccc(-c5cccc6sc7ccccc7c56)cc4)c4cccc5c4oc4c(-c6ccccc6)cccc45)ccc3c2)cc1 ; c1ccc(-c2cccc3c2oc2c(N(c4ccc(-c5cccc6sc7ccccc7c56)cc4)c4cccc(-c5cccc6ccccc56)c4)cccc23)cc1 ; c1ccc(-c2cccc3c2oc2c(N(c4ccc(-c5cccc6sc7ccccc7c56)cc4)c4ccccc4-c4ccc5ccccc5c4)cccc23)cc1 ; c1ccc(-c2cccc3c2oc2c(N(c4ccc(-c5cccc6sc7ccccc7c56)cc4)c4ccc(-c5cccc6ccccc56)c5ccccc45)cccc23)cc1]

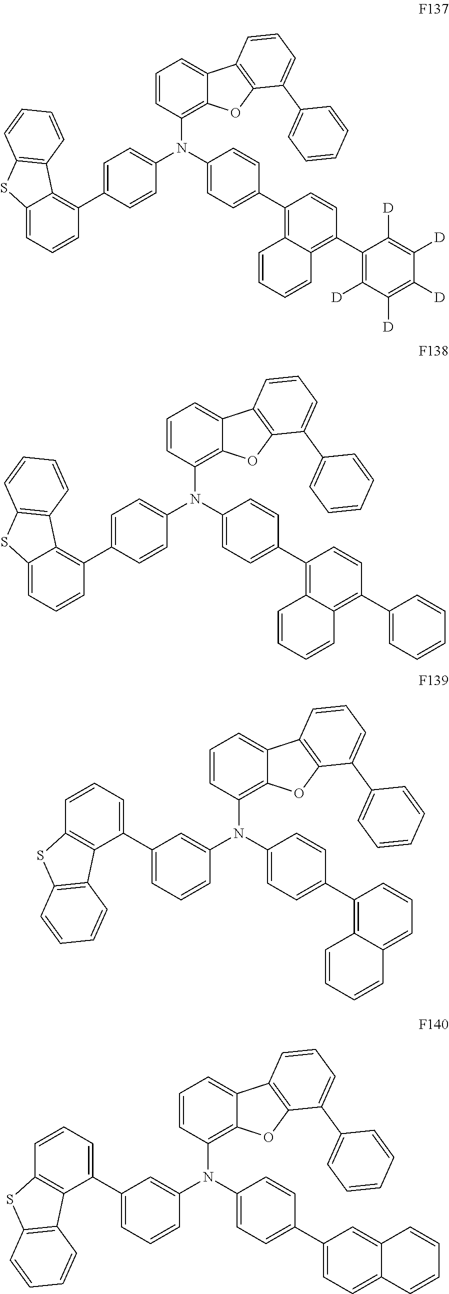 [SMILES: [2H]c1c([2H])c([2H])c(-c2ccc(-c3ccc(N(c4ccc(-c5cccc6sc7ccccc7c56)cc4)c4cccc5c4oc4c(-c6ccccc6)cccc45)cc3)c3ccccc23)c([2H])c1[2H] ; c1ccc(-c2ccc(-c3ccc(N(c4ccc(-c5cccc6sc7ccccc7c56)cc4)c4cccc5c4oc4c(-c6ccccc6)cccc45)cc3)c3ccccc23)cc1 ; c1ccc(-c2cccc3c2oc2c(N(c4ccc(-c5cccc6ccccc56)cc4)c4cccc(-c5cccc6sc7ccccc7c56)c4)cccc23)cc1 ; c1ccc(-c2cccc3c2oc2c(N(c4ccc(-c5ccc6ccccc6c5)cc4)c4cccc(-c5cccc6sc7ccccc7c56)c4)cccc23)cc1]

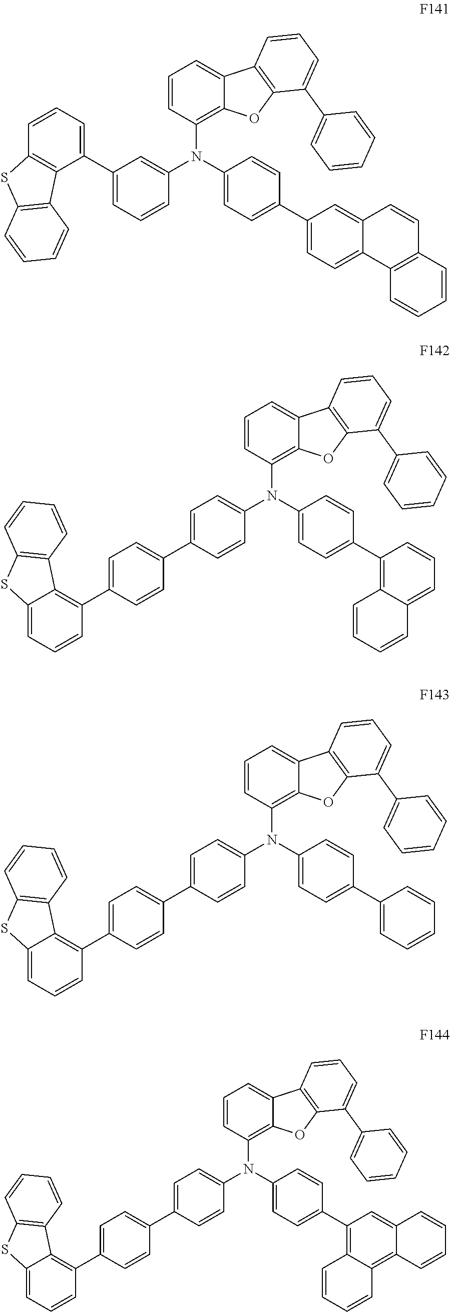 [SMILES: c1ccc(-c2cccc3c2oc2c(N(c4ccc(-c5ccc6c(ccc7ccccc76)c5)cc4)c4cccc(-c5cccc6sc7ccccc7c56)c4)cccc23)cc1 ; c1ccc(-c2cccc3c2oc2c(N(c4ccc(-c5ccc(-c6cccc7sc8ccccc8c67)cc5)cc4)c4ccc(-c5cccc6ccccc56)cc4)cccc23)cc1 ; c1ccc(-c2ccc(N(c3ccc(-c4ccc(-c5cccc6sc7ccccc7c56)cc4)cc3)c3cccc4c3oc3c(-c5ccccc5)cccc34)cc2)cc1 ; c1ccc(-c2cccc3c2oc2c(N(c4ccc(-c5ccc(-c6cccc7sc8ccccc8c67)cc5)cc4)c4ccc(-c5cc6ccccc6c6ccccc56)cc4)cccc23)cc1]

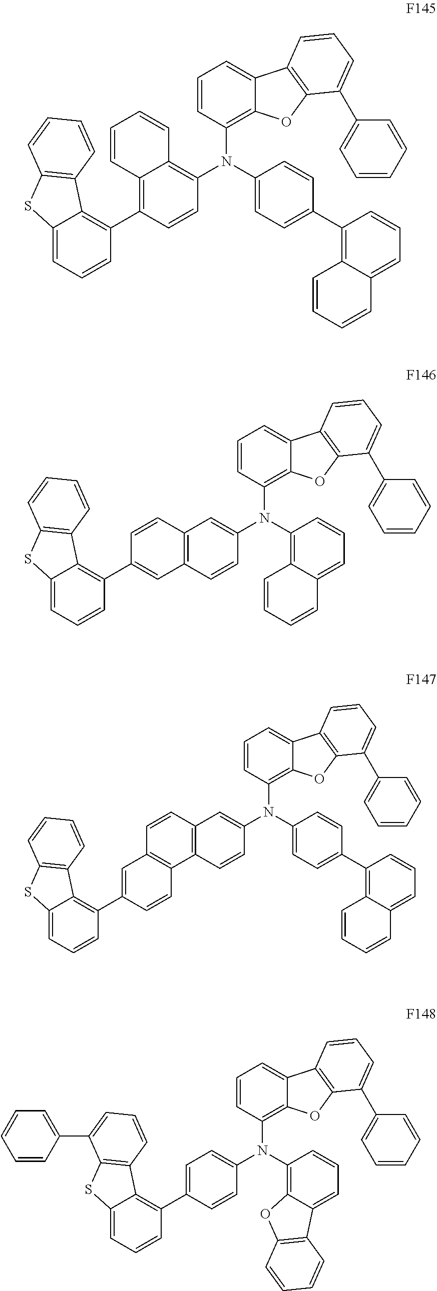 [SMILES: c1ccc(-c2cccc3c2oc2c(N(c4ccc(-c5cccc6ccccc56)cc4)c4ccc(-c5cccc6sc7ccccc7c56)c5ccccc45)cccc23)cc1 ; c1ccc(-c2cccc3c2oc2c(N(c4ccc5cc(-c6cccc7sc8ccccc8c67)ccc5c4)c4cccc5ccccc45)cccc23)cc1 ; c1ccc(-c2cccc3c2oc2c(N(c4ccc(-c5cccc6ccccc56)cc4)c4ccc5c(ccc6cc(-c7cccc8sc9ccccc9c78)ccc65)c4)cccc23)cc1 ; c1ccc(-c2cccc3c2oc2c(N(c4ccc(-c5cccc6sc7c(-c8ccccc8)cccc7c56)cc4)c4cccc5c4oc4ccccc45)cccc23)cc1]

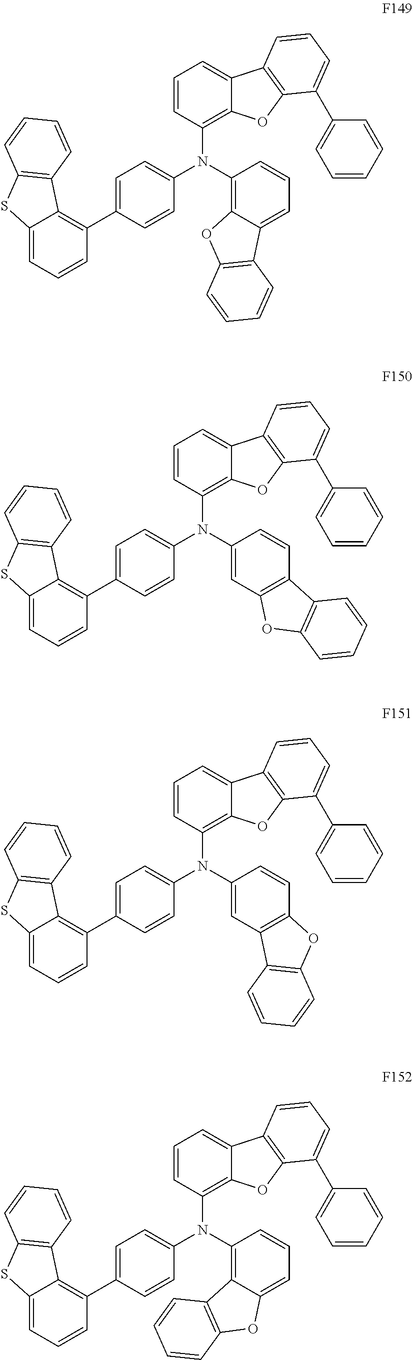 [SMILES: c1ccc(-c2cccc3c2oc2c(N(c4ccc(-c5cccc6sc7ccccc7c56)cc4)c4cccc5c4oc4ccccc45)cccc23)cc1 ; c1ccc(-c2cccc3c2oc2c(N(c4ccc(-c5cccc6sc7ccccc7c56)cc4)c4ccc5c(c4)oc4ccccc45)cccc23)cc1 ; c1ccc(-c2cccc3c2oc2c(N(c4ccc(-c5cccc6sc7ccccc7c56)cc4)c4ccc5oc6ccccc6c5c4)cccc23)cc1 ; c1ccc(-c2cccc3c2oc2c(N(c4ccc(-c5cccc6sc7ccccc7c56)cc4)c4cccc5oc6ccccc6c45)cccc23)cc1]

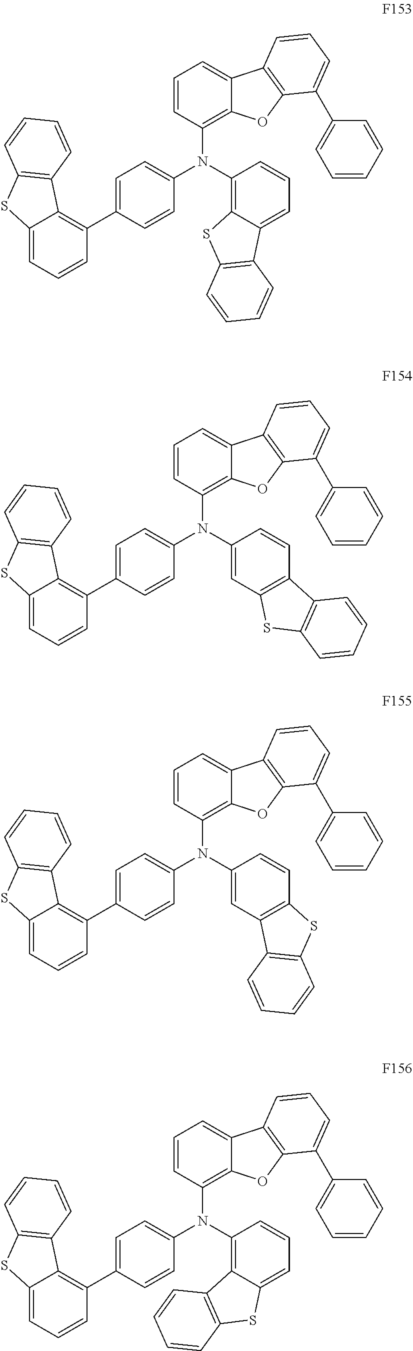 [SMILES: c1ccc(-c2cccc3c2oc2c(N(c4ccc(-c5cccc6sc7ccccc7c56)cc4)c4cccc5c4sc4ccccc45)cccc23)cc1 ; c1ccc(-c2cccc3c2oc2c(N(c4ccc(-c5cccc6sc7ccccc7c56)cc4)c4ccc5c(c4)sc4ccccc45)cccc23)cc1 ; c1ccc(-c2cccc3c2oc2c(N(c4ccc(-c5cccc6sc7ccccc7c56)cc4)c4ccc5sc6ccccc6c5c4)cccc23)cc1 ; c1ccc(-c2cccc3c2oc2c(N(c4ccc(-c5cccc6sc7ccccc7c56)cc4)c4cccc5sc6ccccc6c45)cccc23)cc1]

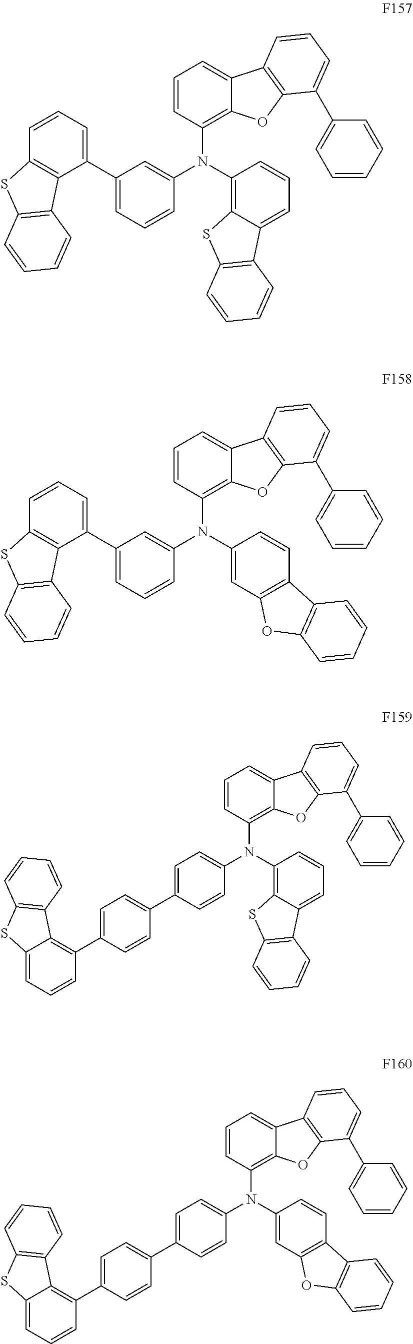 [SMILES: c1ccc(-c2cccc3c2oc2c(N(c4cccc(-c5cccc6sc7ccccc7c56)c4)c4cccc5c4sc4ccccc45)cccc23)cc1 ; c1ccc(-c2cccc3c2oc2c(N(c4cccc(-c5cccc6sc7ccccc7c56)c4)c4ccc5c(c4)oc4ccccc45)cccc23)cc1 ; c1ccc(-c2cccc3c2oc2c(N(c4ccc(-c5ccc(-c6cccc7sc8ccccc8c67)cc5)cc4)c4cccc5c4sc4ccccc45)cccc23)cc1 ; c1ccc(-c2cccc3c2oc2c(N(c4ccc(-c5ccc(-c6cccc7sc8ccccc8c67)cc5)cc4)c4ccc5c(c4)oc4ccccc45)cccc23)cc1]

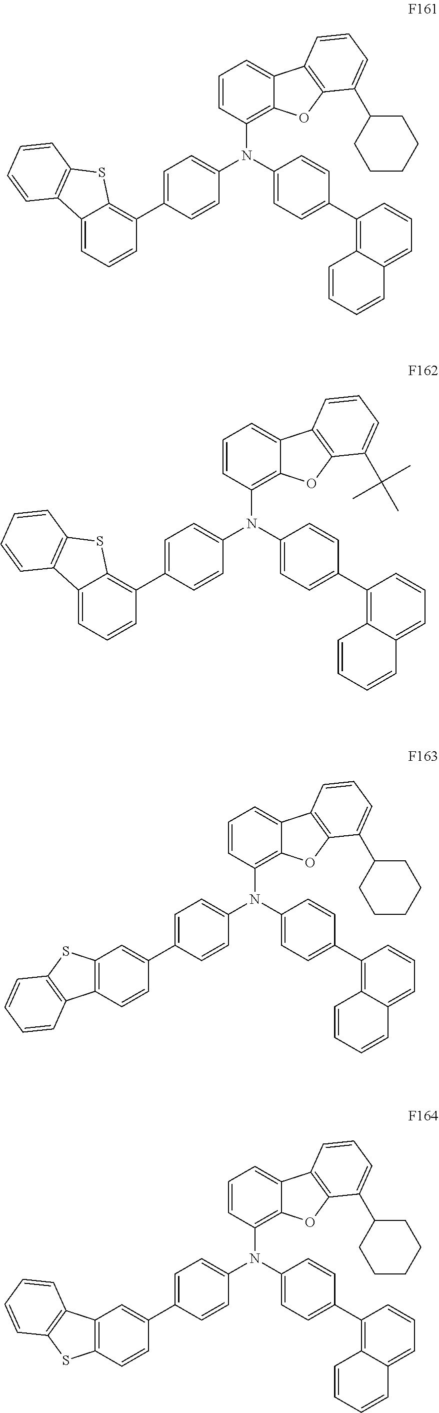 [SMILES: c1ccc2c(-c3ccc(N(c4ccc(-c5cccc6c5sc5ccccc56)cc4)c4cccc5c4oc4c(C6CCCCC6)cccc45)cc3)cccc2c1 ; CC(C)(C)c1cccc2c1oc1c(N(c3ccc(-c4cccc5ccccc45)cc3)c3ccc(-c4cccc5c4sc4ccccc45)cc3)cccc12 ; c1ccc2c(-c3ccc(N(c4ccc(-c5ccc6c(c5)sc5ccccc56)cc4)c4cccc5c4oc4c(C6CCCCC6)cccc45)cc3)cccc2c1 ; c1ccc2c(-c3ccc(N(c4ccc(-c5ccc6sc7ccccc7c6c5)cc4)c4cccc5c4oc4c(C6CCCCC6)cccc45)cc3)cccc2c1]

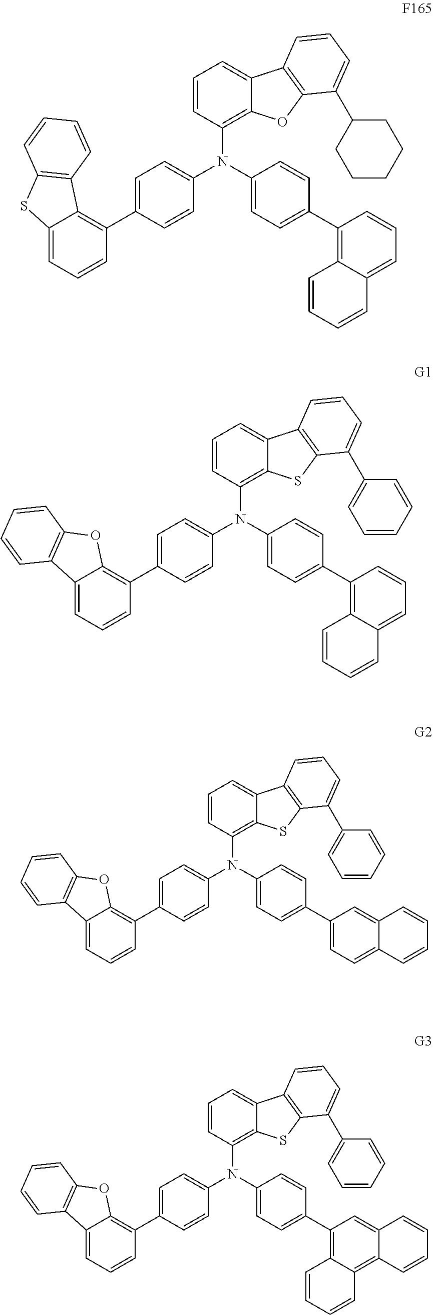 [SMILES: c1ccc2c(-c3ccc(N(c4ccc(-c5cccc6sc7ccccc7c56)cc4)c4cccc5c4oc4c(C6CCCCC6)cccc45)cc3)cccc2c1 ; c1ccc(-c2cccc3c2sc2c(N(c4ccc(-c5cccc6ccccc56)cc4)c4ccc(-c5cccc6c5oc5ccccc56)cc4)cccc23)cc1 ; c1ccc(-c2cccc3c2sc2c(N(c4ccc(-c5ccc6ccccc6c5)cc4)c4ccc(-c5cccc6c5oc5ccccc56)cc4)cccc23)cc1 ; c1ccc(-c2cccc3c2sc2c(N(c4ccc(-c5cc6ccccc6c6ccccc56)cc4)c4ccc(-c5cccc6c5oc5ccccc56)cc4)cccc23)cc1]

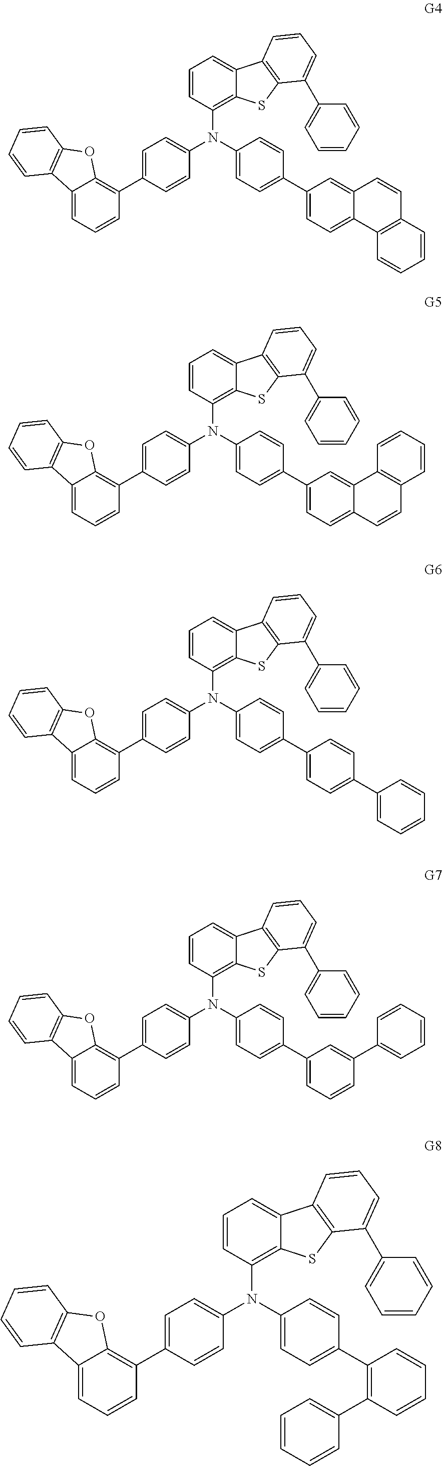 [SMILES: c1ccc(-c2cccc3c2sc2c(N(c4ccc(-c5ccc6c(ccc7ccccc76)c5)cc4)c4ccc(-c5cccc6c5oc5ccccc56)cc4)cccc23)cc1 ; c1ccc(-c2cccc3c2sc2c(N(c4ccc(-c5ccc6ccc7ccccc7c6c5)cc4)c4ccc(-c5cccc6c5oc5ccccc56)cc4)cccc23)cc1 ; c1ccc(-c2ccc(-c3ccc(N(c4ccc(-c5cccc6c5oc5ccccc56)cc4)c4cccc5c4sc4c(-c6ccccc6)cccc45)cc3)cc2)cc1 ; c1ccc(-c2cccc(-c3ccc(N(c4ccc(-c5cccc6c5oc5ccccc56)cc4)c4cccc5c4sc4c(-c6ccccc6)cccc45)cc3)c2)cc1 ; c1ccc(-c2ccccc2-c2ccc(N(c3ccc(-c4cccc5c4oc4ccccc45)cc3)c3cccc4c3sc3c(-c5ccccc5)cccc34)cc2)cc1]

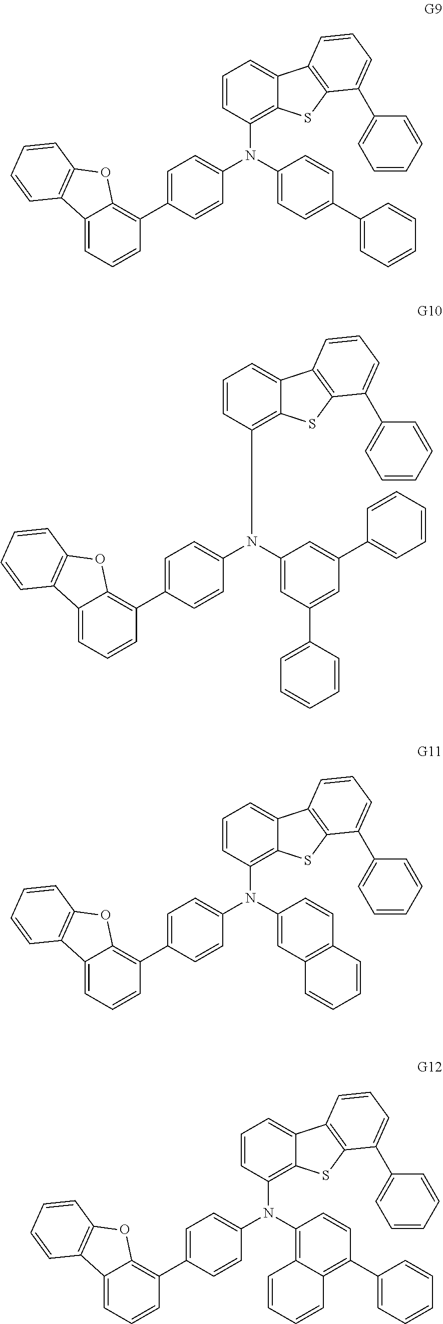 [SMILES: c1ccc(-c2ccc(N(c3ccc(-c4cccc5c4oc4ccccc45)cc3)c3cccc4c3sc3c(-c5ccccc5)cccc34)cc2)cc1 ; c1ccc(-c2cc(-c3ccccc3)cc(N(c3ccc(-c4cccc5c4oc4ccccc45)cc3)c3cccc4c3sc3c(-c5ccccc5)cccc34)c2)cc1 ; c1ccc(-c2cccc3c2sc2c(N(c4ccc(-c5cccc6c5oc5ccccc56)cc4)c4ccc5ccccc5c4)cccc23)cc1 ; c1ccc(-c2ccc(N(c3ccc(-c4cccc5c4oc4ccccc45)cc3)c3cccc4c3sc3c(-c5ccccc5)cccc34)c3ccccc23)cc1]

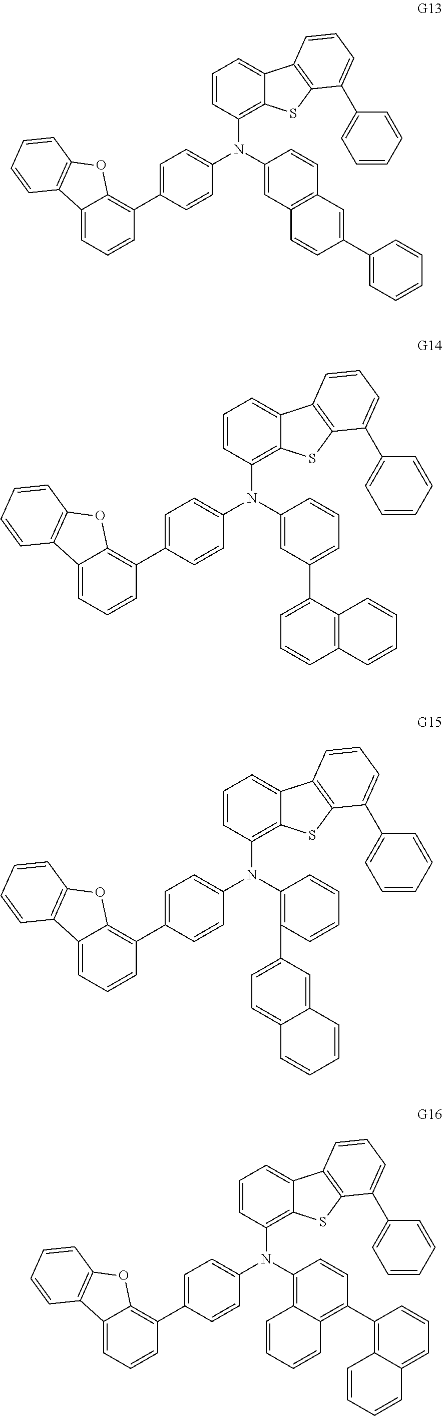 [SMILES: c1ccc(-c2ccc3cc(N(c4ccc(-c5cccc6c5oc5ccccc56)cc4)c4cccc5c4sc4c(-c6ccccc6)cccc45)ccc3c2)cc1 ; c1ccc(-c2cccc3c2sc2c(N(c4ccc(-c5cccc6c5oc5ccccc56)cc4)c4cccc(-c5cccc6ccccc56)c4)cccc23)cc1 ; c1ccc(-c2cccc3c2sc2c(N(c4ccc(-c5cccc6c5oc5ccccc56)cc4)c4ccccc4-c4ccc5ccccc5c4)cccc23)cc1 ; c1ccc(-c2cccc3c2sc2c(N(c4ccc(-c5cccc6c5oc5ccccc56)cc4)c4ccc(-c5cccc6ccccc56)c5ccccc45)cccc23)cc1]

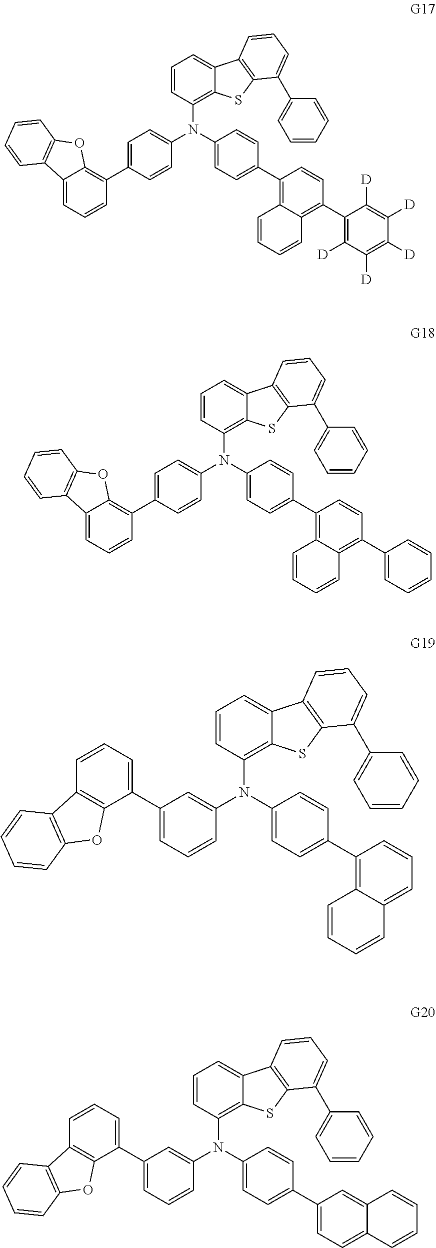 [SMILES: [2H]c1c([2H])c([2H])c(-c2ccc(-c3ccc(N(c4ccc(-c5cccc6c5oc5ccccc56)cc4)c4cccc5c4sc4c(-c6ccccc6)cccc45)cc3)c3ccccc23)c([2H])c1[2H] ; c1ccc(-c2ccc(-c3ccc(N(c4ccc(-c5cccc6c5oc5ccccc56)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4cccc(-c5cccc6c5oc5ccccc56)c4)cccc23)cc1 ; c1ccc(-c2cccc3c2sc2c(N(c4ccc(-c5ccc6ccccc6c5)cc4)c4cccc(-c5cccc6c5oc5ccccc56)c4)cccc23)cc1]

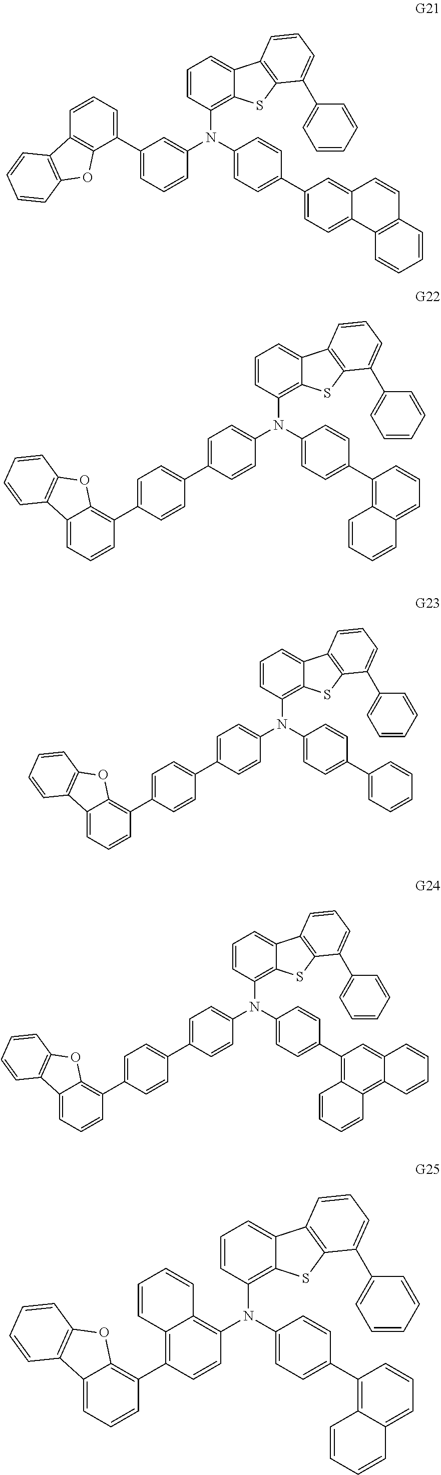

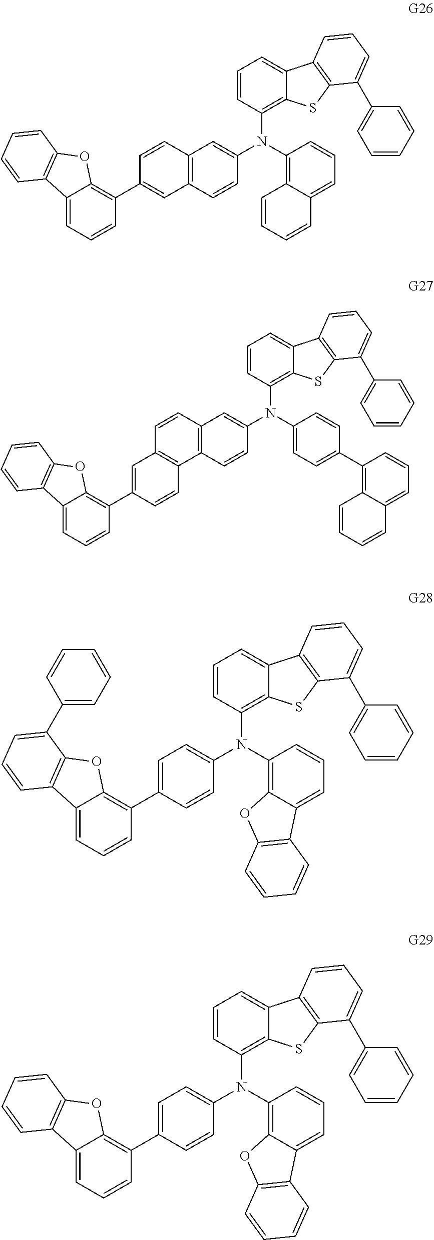 [SMILES: c1ccc(-c2cccc3c2sc2c(N(c4ccc5cc(-c6cccc7c6oc6ccccc67)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7cccc8c7oc7ccccc78)ccc65)c4)cccc23)cc1 ; c1ccc(-c2cccc3c2oc2c(-c4ccc(N(c5cccc6c5oc5ccccc56)c5cccc6c5sc5c(-c7ccccc7)cccc56)cc4)cccc23)cc1 ; c1ccc(-c2cccc3c2sc2c(N(c4ccc(-c5cccc6c5oc5ccccc56)cc4)c4cccc5c4oc4ccccc45)cccc23)cc1]

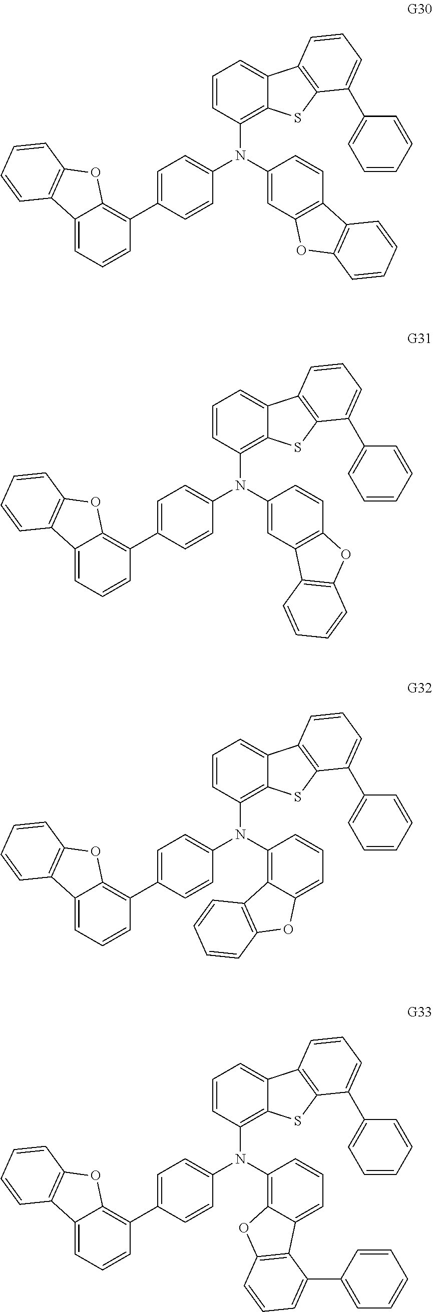 [SMILES: c1ccc(-c2cccc3c2sc2c(N(c4ccc(-c5cccc6c5oc5ccccc56)cc4)c4ccc5c(c4)oc4ccccc45)cccc23)cc1 ; c1ccc(-c2cccc3c2sc2c(N(c4ccc(-c5cccc6c5oc5ccccc56)cc4)c4ccc5oc6ccccc6c5c4)cccc23)cc1 ; c1ccc(-c2cccc3c2sc2c(N(c4ccc(-c5cccc6c5oc5ccccc56)cc4)c4cccc5oc6ccccc6c45)cccc23)cc1 ; c1ccc(-c2cccc3c2sc2c(N(c4ccc(-c5cccc6c5oc5ccccc56)cc4)c4cccc5c4oc4cccc(-c6ccccc6)c45)cccc23)cc1]

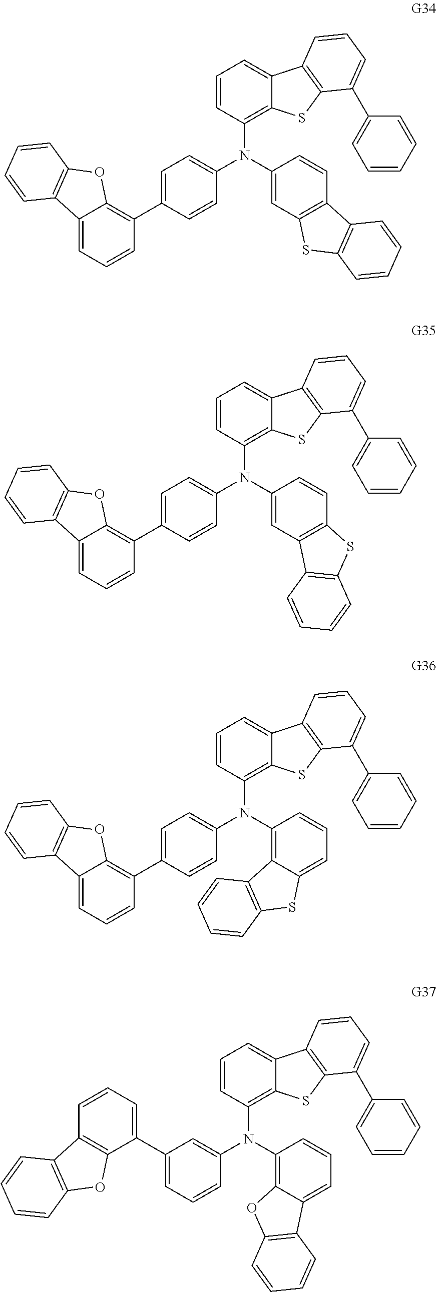 [SMILES: c1ccc(-c2cccc3c2sc2c(N(c4ccc(-c5cccc6c5oc5ccccc56)cc4)c4ccc5c(c4)sc4ccccc45)cccc23)cc1 ; c1ccc(-c2cccc3c2sc2c(N(c4ccc(-c5cccc6c5oc5ccccc56)cc4)c4ccc5sc6ccccc6c5c4)cccc23)cc1 ; c1ccc(-c2cccc3c2sc2c(N(c4ccc(-c5cccc6c5oc5ccccc56)cc4)c4cccc5sc6ccccc6c45)cccc23)cc1 ; c1ccc(-c2cccc3c2sc2c(N(c4cccc(-c5cccc6c5oc5ccccc56)c4)c4cccc5c4oc4ccccc45)cccc23)cc1]

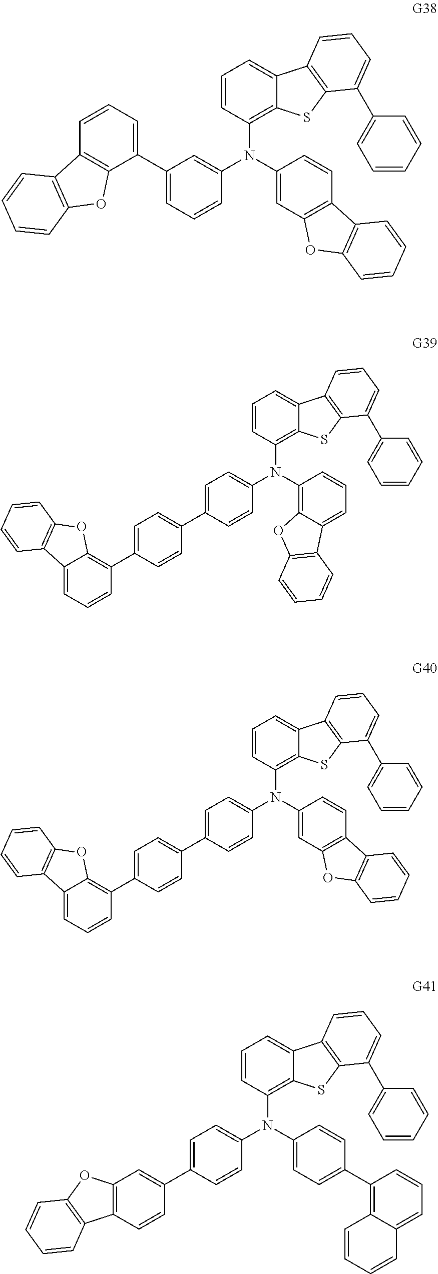 [SMILES: c1ccc(-c2cccc3c2sc2c(N(c4cccc(-c5cccc6c5oc5ccccc56)c4)c4ccc5c(c4)oc4ccccc45)cccc23)cc1 ; c1ccc(-c2cccc3c2sc2c(N(c4ccc(-c5ccc(-c6cccc7c6oc6ccccc67)cc5)cc4)c4cccc5c4oc4ccccc45)cccc23)cc1 ; c1ccc(-c2cccc3c2sc2c(N(c4ccc(-c5ccc(-c6cccc7c6oc6ccccc67)cc5)cc4)c4ccc5c(c4)oc4ccccc45)cccc23)cc1 ; c1ccc(-c2cccc3c2sc2c(N(c4ccc(-c5ccc6c(c5)oc5ccccc56)cc4)c4ccc(-c5cccc6ccccc56)cc4)cccc23)cc1]

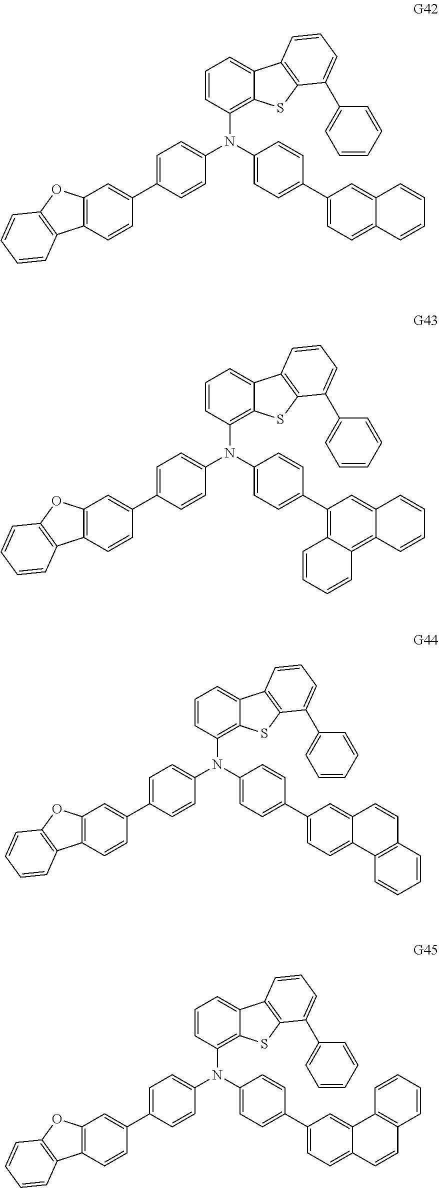 [SMILES: c1ccc(-c2cccc3c2sc2c(N(c4ccc(-c5ccc6ccccc6c5)cc4)c4ccc(-c5ccc6c(c5)oc5ccccc56)cc4)cccc23)cc1 ; c1ccc(-c2cccc3c2sc2c(N(c4ccc(-c5ccc6c(c5)oc5ccccc56)cc4)c4ccc(-c5cc6ccccc6c6ccccc56)cc4)cccc23)cc1 ; c1ccc(-c2cccc3c2sc2c(N(c4ccc(-c5ccc6c(ccc7ccccc76)c5)cc4)c4ccc(-c5ccc6c(c5)oc5ccccc56)cc4)cccc23)cc1 ; c1ccc(-c2cccc3c2sc2c(N(c4ccc(-c5ccc6c(c5)oc5ccccc56)cc4)c4ccc(-c5ccc6ccc7ccccc7c6c5)cc4)cccc23)cc1]

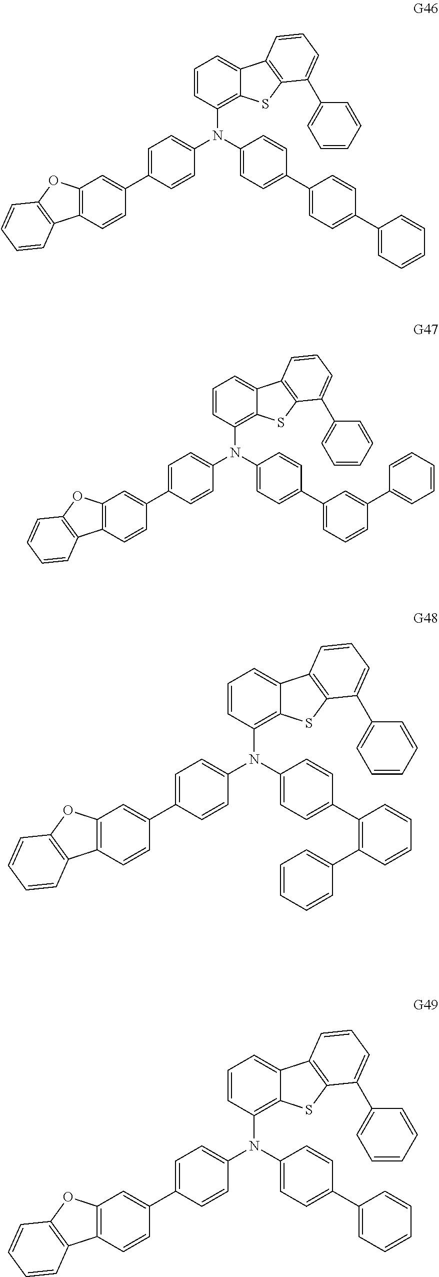 [SMILES: c1ccc(-c2ccc(-c3ccc(N(c4ccc(-c5ccc6c(c5)oc5ccccc56)cc4)c4cccc5c4sc4c(-c6ccccc6)cccc45)cc3)cc2)cc1 ; c1ccc(-c2cccc(-c3ccc(N(c4ccc(-c5ccc6c(c5)oc5ccccc56)cc4)c4cccc5c4sc4c(-c6ccccc6)cccc45)cc3)c2)cc1 ; c1ccc(-c2ccccc2-c2ccc(N(c3ccc(-c4ccc5c(c4)oc4ccccc45)cc3)c3cccc4c3sc3c(-c5ccccc5)cccc34)cc2)cc1 ; c1ccc(-c2ccc(N(c3ccc(-c4ccc5c(c4)oc4ccccc45)cc3)c3cccc4c3sc3c(-c5ccccc5)cccc34)cc2)cc1]

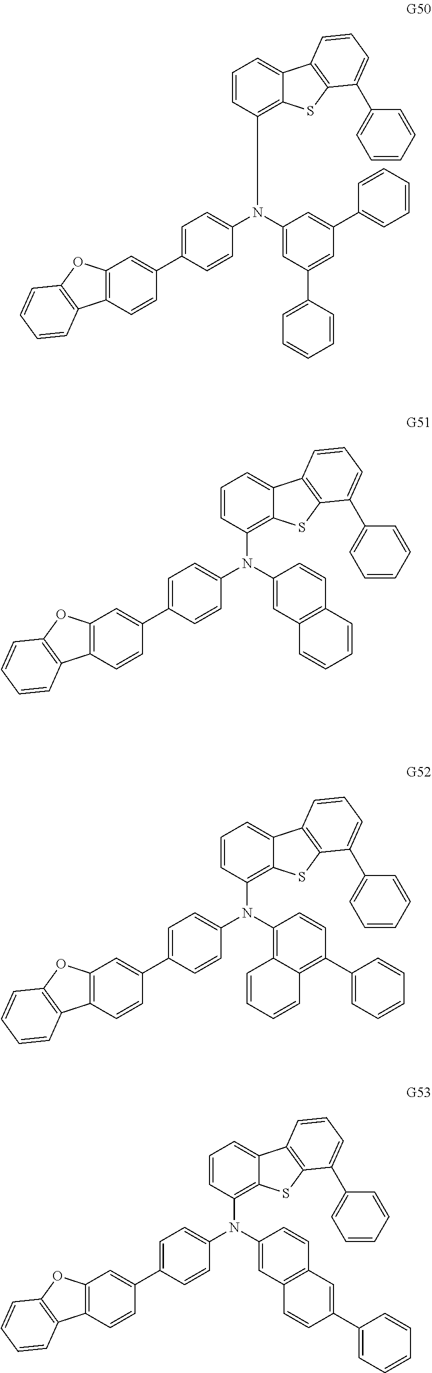 [SMILES: c1ccc(-c2cc(-c3ccccc3)cc(N(c3ccc(-c4ccc5c(c4)oc4ccccc45)cc3)c3cccc4c3sc3c(-c5ccccc5)cccc34)c2)cc1 ; c1ccc(-c2cccc3c2sc2c(N(c4ccc(-c5ccc6c(c5)oc5ccccc56)cc4)c4ccc5ccccc5c4)cccc23)cc1 ; c1ccc(-c2ccc(N(c3ccc(-c4ccc5c(c4)oc4ccccc45)cc3)c3cccc4c3sc3c(-c5ccccc5)cccc34)c3ccccc23)cc1 ; c1ccc(-c2ccc3cc(N(c4ccc(-c5ccc6c(c5)oc5ccccc56)cc4)c4cccc5c4sc4c(-c6ccccc6)cccc45)ccc3c2)cc1]

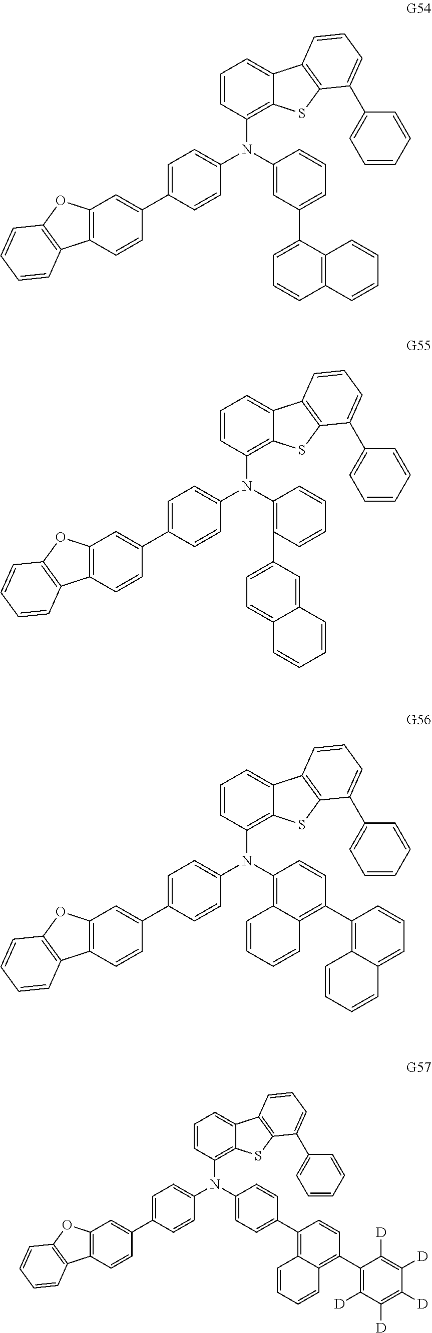 [SMILES: c1ccc(-c2cccc3c2sc2c(N(c4ccc(-c5ccc6c(c5)oc5ccccc56)cc4)c4cccc(-c5cccc6ccccc56)c4)cccc23)cc1 ; c1ccc(-c2cccc3c2sc2c(N(c4ccc(-c5ccc6c(c5)oc5ccccc56)cc4)c4ccccc4-c4ccc5ccccc5c4)cccc23)cc1 ; c1ccc(-c2cccc3c2sc2c(N(c4ccc(-c5ccc6c(c5)oc5ccccc56)cc4)c4ccc(-c5cccc6ccccc56)c5ccccc45)cccc23)cc1 ; [2H]c1c([2H])c([2H])c(-c2ccc(-c3ccc(N(c4ccc(-c5ccc6c(c5)oc5ccccc56)cc4)c4cccc5c4sc4c(-c6ccccc6)cccc45)cc3)c3ccccc23)c([2H])c1[2H]]

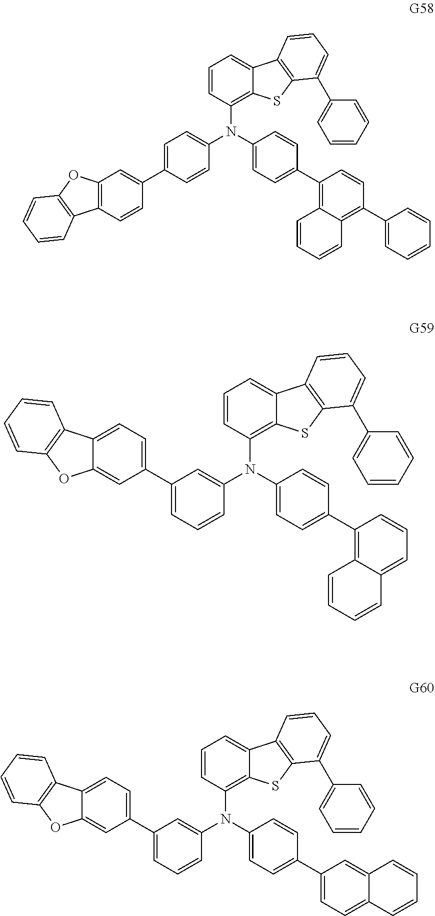 [SMILES: c1ccc(-c2ccc(-c3ccc(N(c4ccc(-c5ccc6c(c5)oc5ccccc56)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4cccc(-c5ccc6c(c5)oc5ccccc56)c4)cccc23)cc1 ; c1ccc(-c2cccc3c2sc2c(N(c4ccc(-c5ccc6ccccc6c5)cc4)c4cccc(-c5ccc6c(c5)oc5ccccc56)c4)cccc23)cc1]

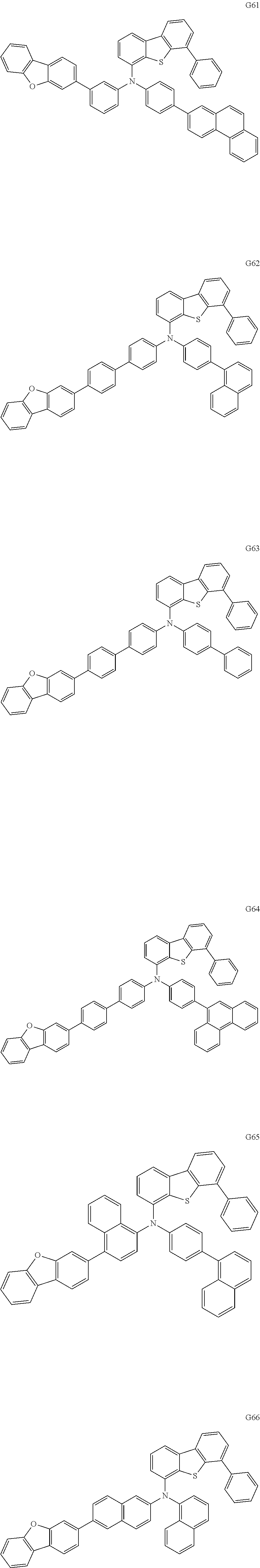

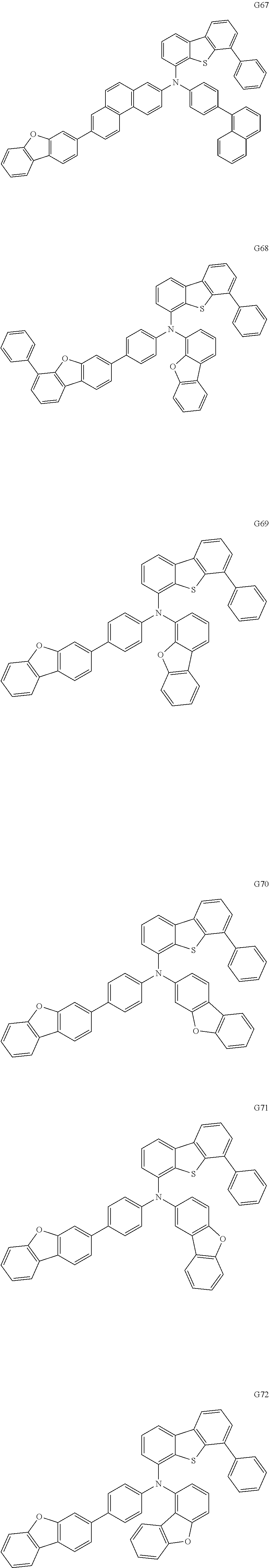

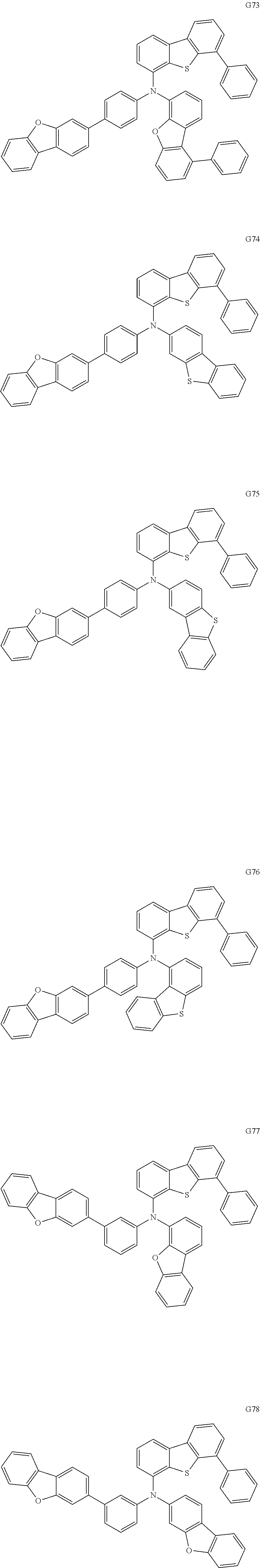

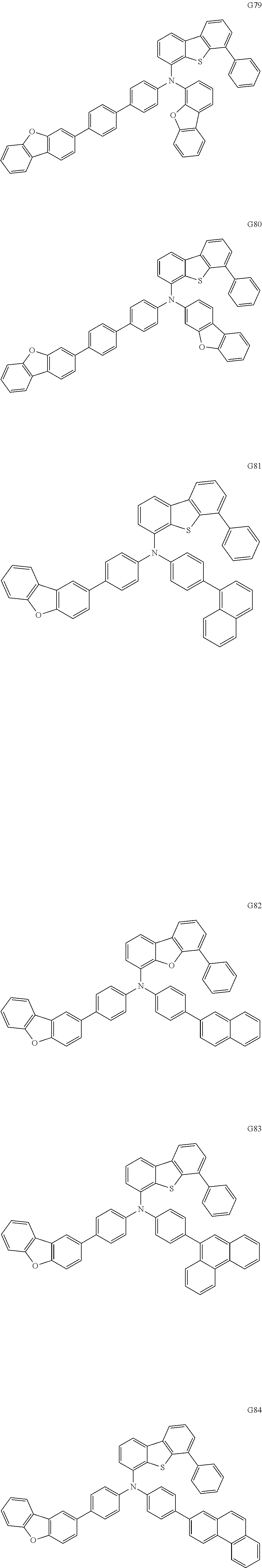

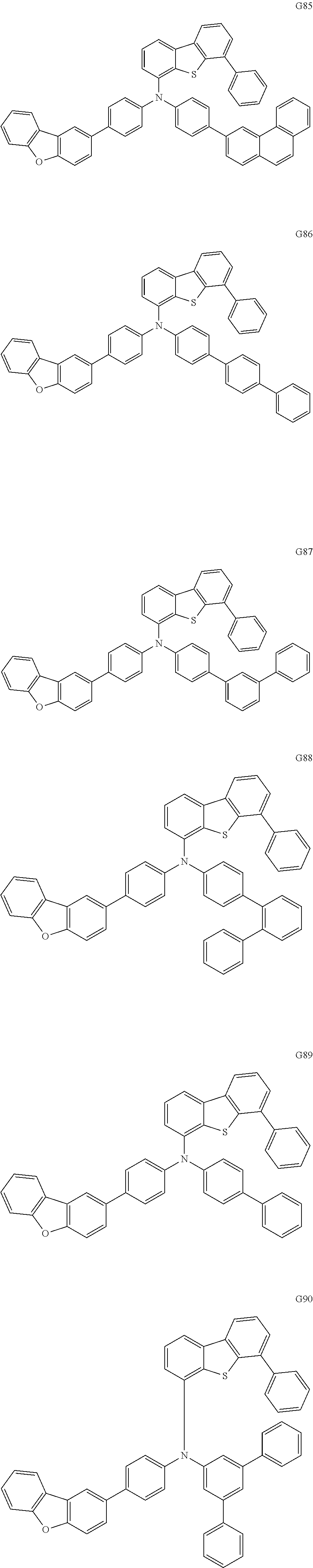

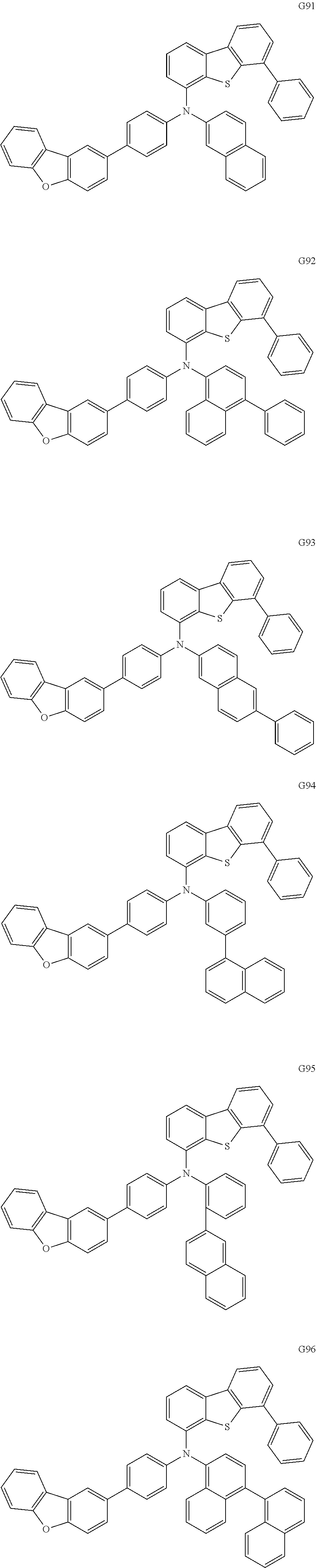

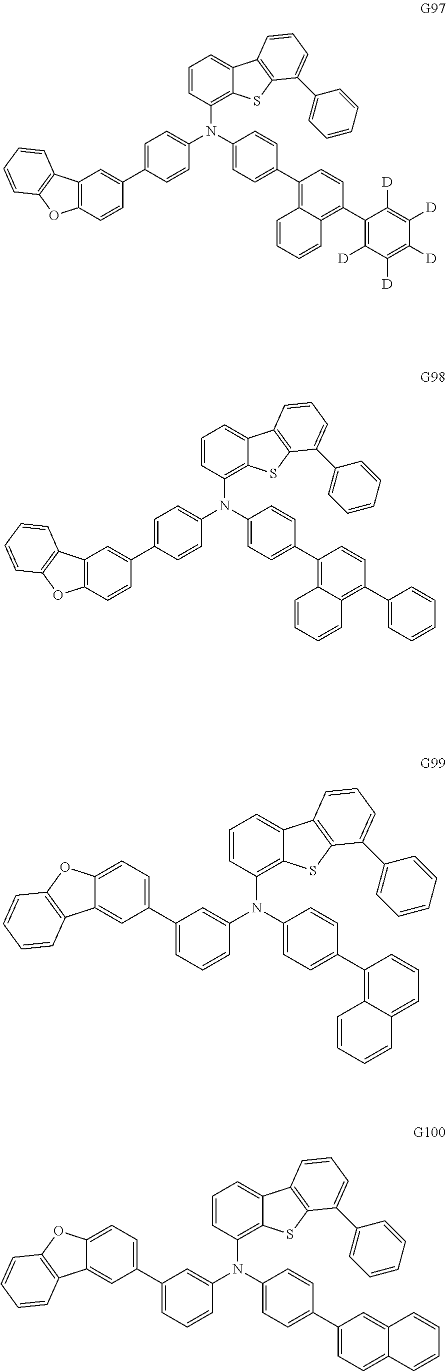 [SMILES: [2H]c1c([2H])c([2H])c(-c2ccc(-c3ccc(N(c4ccc(-c5ccc6oc7ccccc7c6c5)cc4)c4cccc5c4sc4c(-c6ccccc6)cccc45)cc3)c3ccccc23)c([2H])c1[2H] ; c1ccc(-c2ccc(-c3ccc(N(c4ccc(-c5ccc6oc7ccccc7c6c5)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4cccc(-c5ccc6oc7ccccc7c6c5)c4)cccc23)cc1 ; c1ccc(-c2cccc3c2sc2c(N(c4ccc(-c5ccc6ccccc6c5)cc4)c4cccc(-c5ccc6oc7ccccc7c6c5)c4)cccc23)cc1]

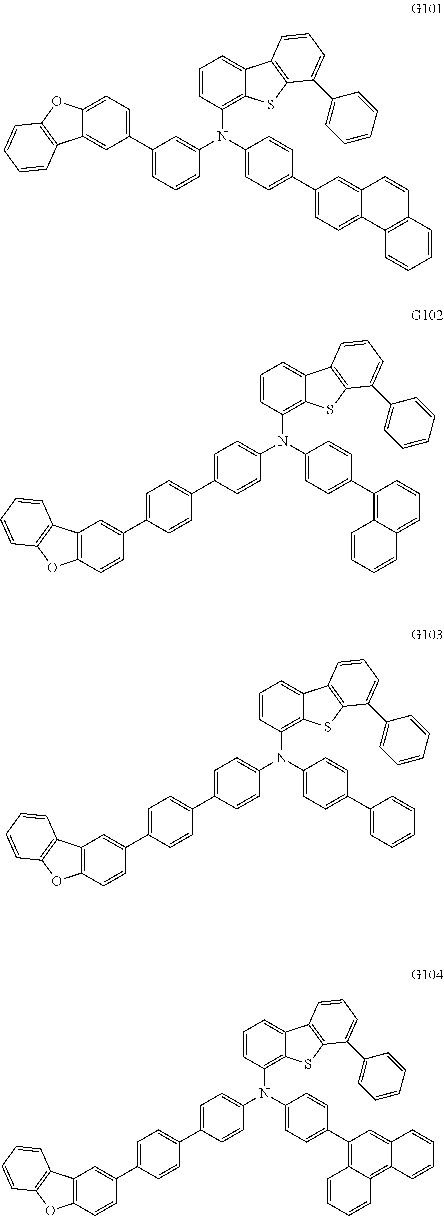 [SMILES: c1ccc(-c2cccc3c2sc2c(N(c4ccc(-c5ccc6c(ccc7ccccc76)c5)cc4)c4cccc(-c5ccc6oc7ccccc7c6c5)c4)cccc23)cc1 ; c1ccc(-c2cccc3c2sc2c(N(c4ccc(-c5ccc(-c6ccc7oc8ccccc8c7c6)cc5)cc4)c4ccc(-c5cccc6ccccc56)cc4)cccc23)cc1 ; c1ccc(-c2ccc(N(c3ccc(-c4ccc(-c5ccc6oc7ccccc7c6c5)cc4)cc3)c3cccc4c3sc3c(-c5ccccc5)cccc34)cc2)cc1 ; c1ccc(-c2cccc3c2sc2c(N(c4ccc(-c5ccc(-c6ccc7oc8ccccc8c7c6)cc5)cc4)c4ccc(-c5cc6ccccc6c6ccccc56)cc4)cccc23)cc1]

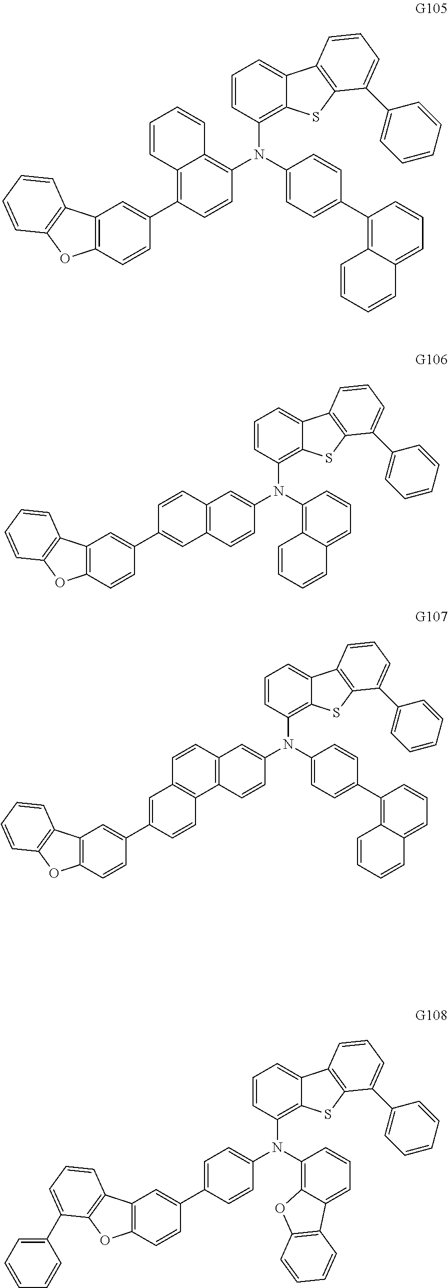 [SMILES: c1ccc(-c2cccc3c2sc2c(N(c4ccc(-c5cccc6ccccc56)cc4)c4ccc(-c5ccc6oc7ccccc7c6c5)c5ccccc45)cccc23)cc1 ; c1ccc(-c2cccc3c2sc2c(N(c4ccc5cc(-c6ccc7oc8ccccc8c7c6)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7ccc8oc9ccccc9c8c7)ccc65)c4)cccc23)cc1 ; c1ccc(-c2cccc3c2oc2ccc(-c4ccc(N(c5cccc6c5oc5ccccc56)c5cccc6c5sc5c(-c7ccccc7)cccc56)cc4)cc23)cc1]

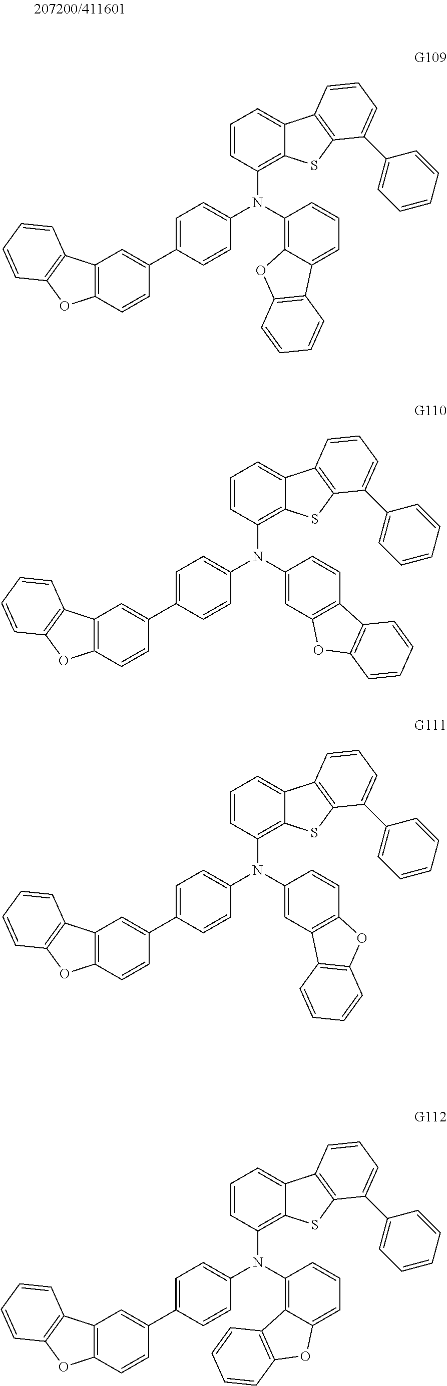 [SMILES: c1ccc(-c2cccc3c2sc2c(N(c4ccc(-c5ccc6oc7ccccc7c6c5)cc4)c4cccc5c4oc4ccccc45)cccc23)cc1 ; c1ccc(-c2cccc3c2sc2c(N(c4ccc(-c5ccc6oc7ccccc7c6c5)cc4)c4ccc5c(c4)oc4ccccc45)cccc23)cc1 ; c1ccc(-c2cccc3c2sc2c(N(c4ccc(-c5ccc6oc7ccccc7c6c5)cc4)c4ccc5oc6ccccc6c5c4)cccc23)cc1 ; c1ccc(-c2cccc3c2sc2c(N(c4ccc(-c5ccc6oc7ccccc7c6c5)cc4)c4cccc5oc6ccccc6c45)cccc23)cc1]

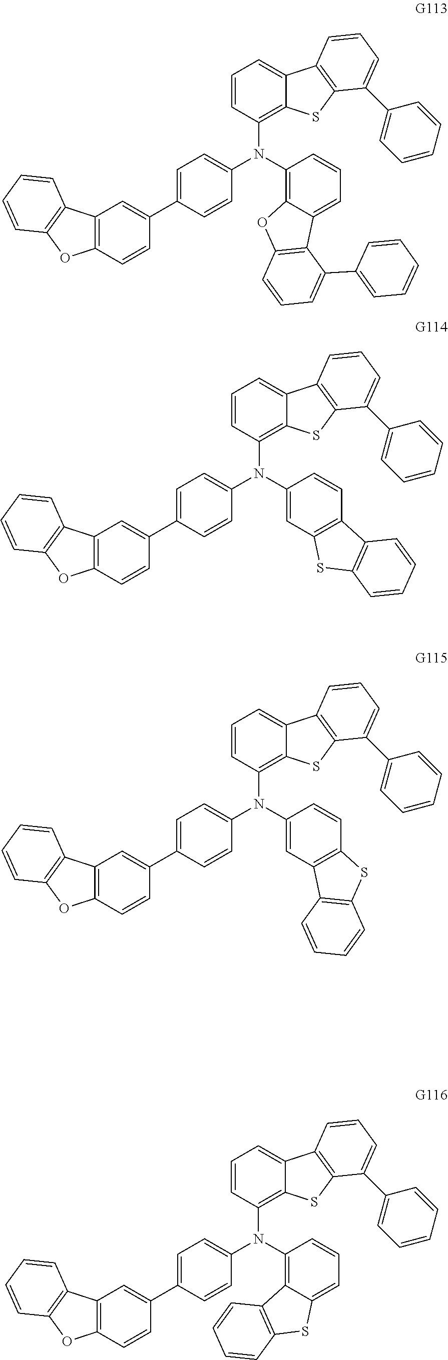 [SMILES: c1ccc(-c2cccc3c2sc2c(N(c4ccc(-c5ccc6oc7ccccc7c6c5)cc4)c4cccc5c4oc4cccc(-c6ccccc6)c45)cccc23)cc1 ; c1ccc(-c2cccc3c2sc2c(N(c4ccc(-c5ccc6oc7ccccc7c6c5)cc4)c4ccc5c(c4)sc4ccccc45)cccc23)cc1 ; c1ccc(-c2cccc3c2sc2c(N(c4ccc(-c5ccc6oc7ccccc7c6c5)cc4)c4ccc5sc6ccccc6c5c4)cccc23)cc1 ; c1ccc(-c2cccc3c2sc2c(N(c4ccc(-c5ccc6oc7ccccc7c6c5)cc4)c4cccc5sc6ccccc6c45)cccc23)cc1]

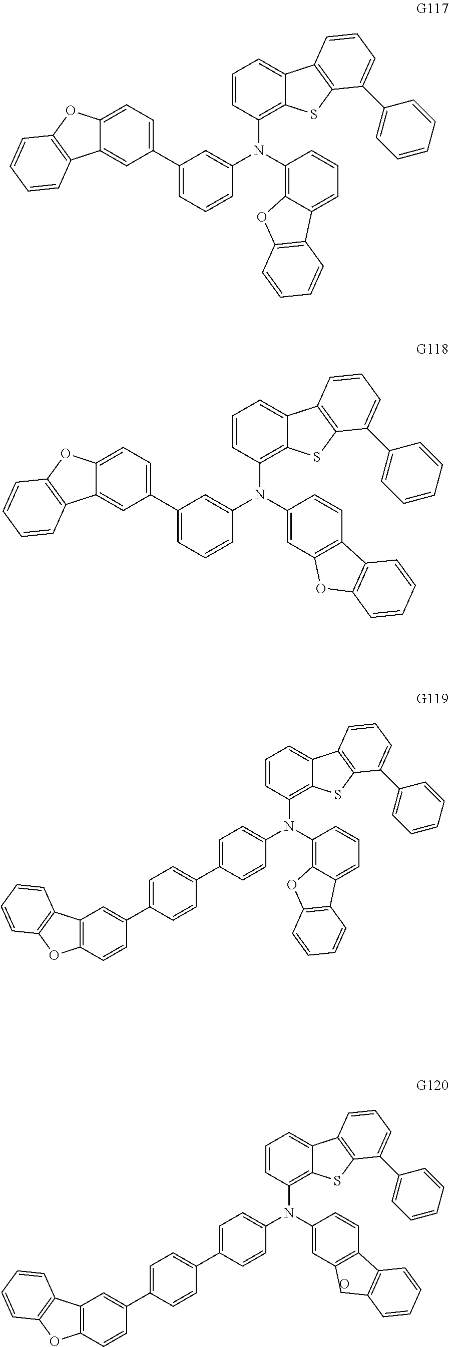

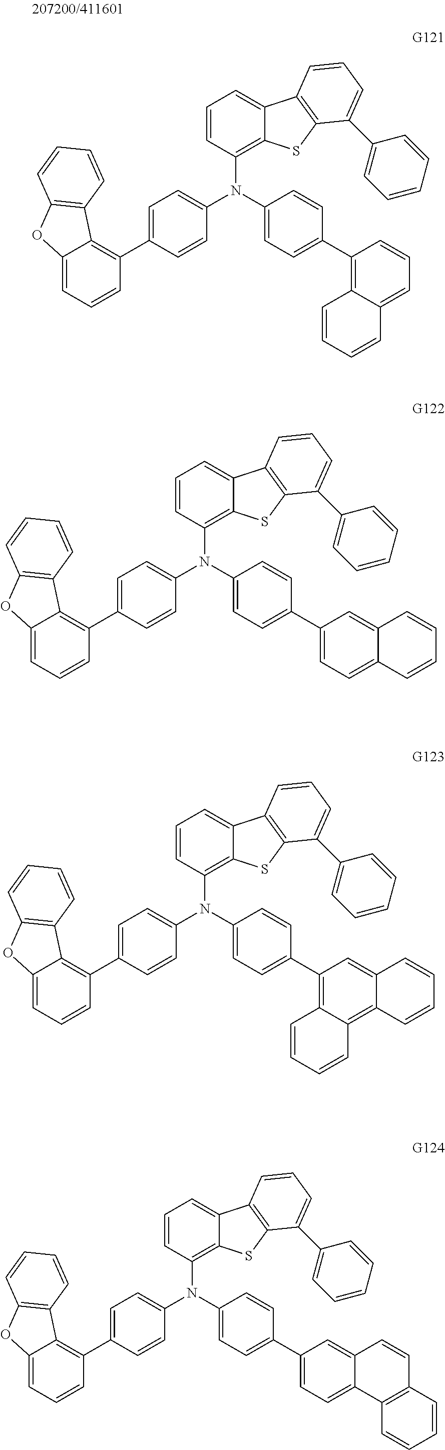 [SMILES: c1ccc(-c2cccc3c2sc2c(N(c4ccc(-c5cccc6ccccc56)cc4)c4ccc(-c5cccc6oc7ccccc7c56)cc4)cccc23)cc1 ; c1ccc(-c2cccc3c2sc2c(N(c4ccc(-c5ccc6ccccc6c5)cc4)c4ccc(-c5cccc6oc7ccccc7c56)cc4)cccc23)cc1 ; c1ccc(-c2cccc3c2sc2c(N(c4ccc(-c5cc6ccccc6c6ccccc56)cc4)c4ccc(-c5cccc6oc7ccccc7c56)cc4)cccc23)cc1 ; c1ccc(-c2cccc3c2sc2c(N(c4ccc(-c5ccc6c(ccc7ccccc76)c5)cc4)c4ccc(-c5cccc6oc7ccccc7c56)cc4)cccc23)cc1]

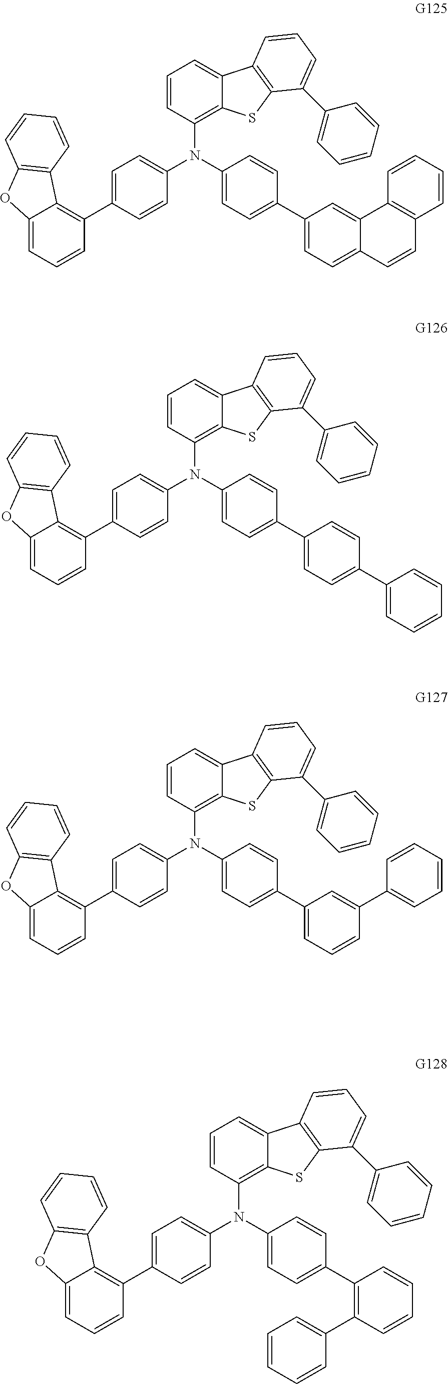 [SMILES: c1ccc(-c2cccc3c2sc2c(N(c4ccc(-c5ccc6ccc7ccccc7c6c5)cc4)c4ccc(-c5cccc6oc7ccccc7c56)cc4)cccc23)cc1 ; c1ccc(-c2ccc(-c3ccc(N(c4ccc(-c5cccc6oc7ccccc7c56)cc4)c4cccc5c4sc4c(-c6ccccc6)cccc45)cc3)cc2)cc1 ; c1ccc(-c2cccc(-c3ccc(N(c4ccc(-c5cccc6oc7ccccc7c56)cc4)c4cccc5c4sc4c(-c6ccccc6)cccc45)cc3)c2)cc1 ; c1ccc(-c2ccccc2-c2ccc(N(c3ccc(-c4cccc5oc6ccccc6c45)cc3)c3cccc4c3sc3c(-c5ccccc5)cccc34)cc2)cc1]

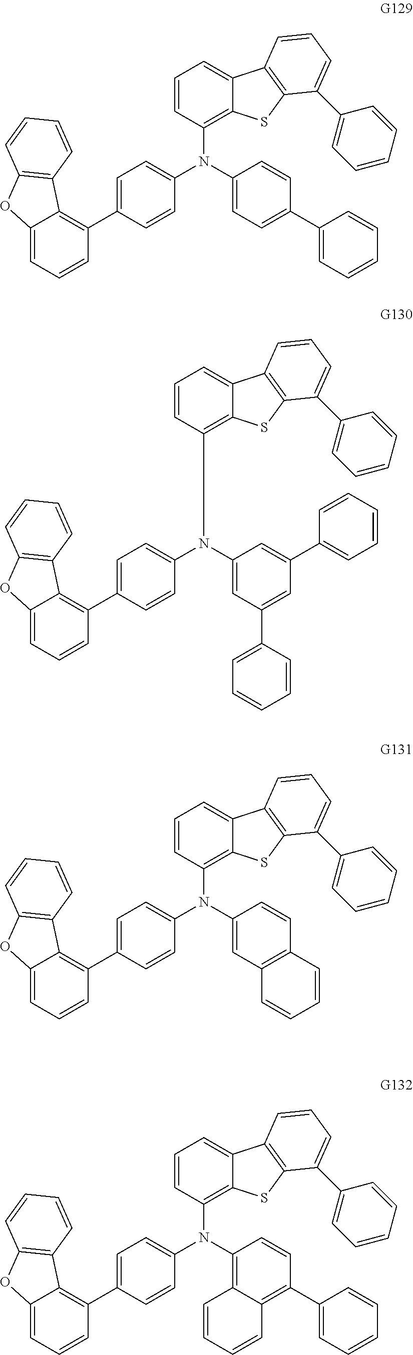 [SMILES: c1ccc(-c2ccc(N(c3ccc(-c4cccc5oc6ccccc6c45)cc3)c3cccc4c3sc3c(-c5ccccc5)cccc34)cc2)cc1 ; c1ccc(-c2cc(-c3ccccc3)cc(N(c3ccc(-c4cccc5oc6ccccc6c45)cc3)c3cccc4c3sc3c(-c5ccccc5)cccc34)c2)cc1 ; c1ccc(-c2cccc3c2sc2c(N(c4ccc(-c5cccc6oc7ccccc7c56)cc4)c4ccc5ccccc5c4)cccc23)cc1 ; c1ccc(-c2ccc(N(c3ccc(-c4cccc5oc6ccccc6c45)cc3)c3cccc4c3sc3c(-c5ccccc5)cccc34)c3ccccc23)cc1]

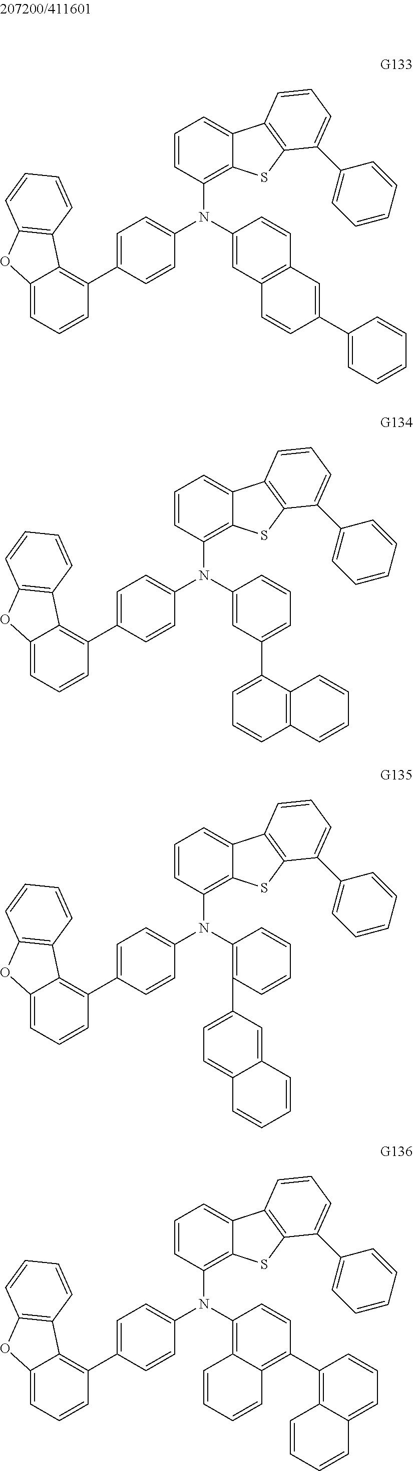 [SMILES: c1ccc(-c2ccc3cc(N(c4ccc(-c5cccc6oc7ccccc7c56)cc4)c4cccc5c4sc4c(-c6ccccc6)cccc45)ccc3c2)cc1 ; c1ccc(-c2cccc3c2sc2c(N(c4ccc(-c5cccc6oc7ccccc7c56)cc4)c4cccc(-c5cccc6ccccc56)c4)cccc23)cc1 ; c1ccc(-c2cccc3c2sc2c(N(c4ccc(-c5cccc6oc7ccccc7c56)cc4)c4ccccc4-c4ccc5ccccc5c4)cccc23)cc1 ; c1ccc(-c2cccc3c2sc2c(N(c4ccc(-c5cccc6oc7ccccc7c56)cc4)c4ccc(-c5cccc6ccccc56)c5ccccc45)cccc23)cc1]

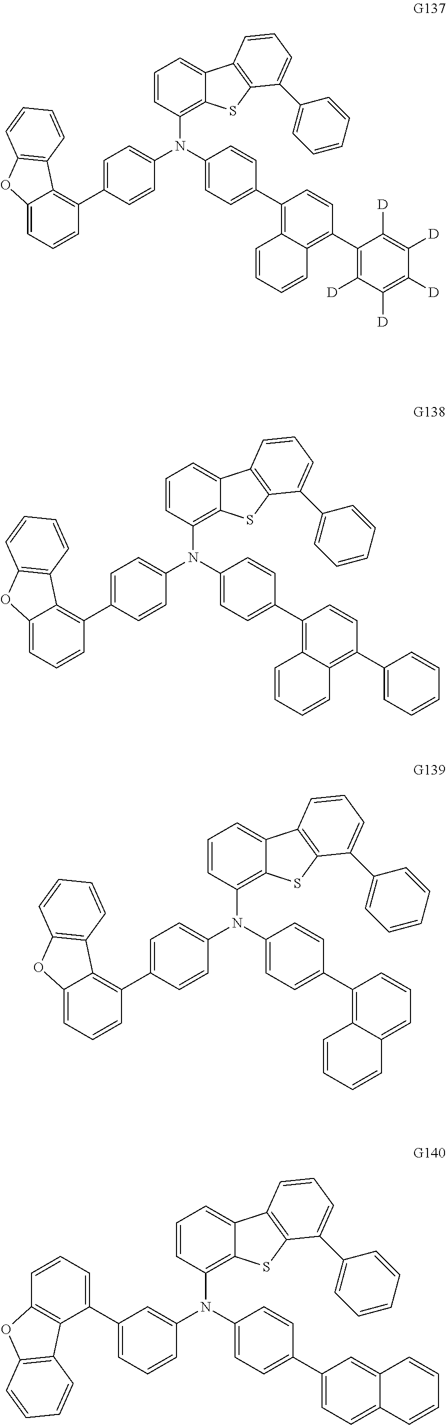 [SMILES: [2H]c1c([2H])c([2H])c(-c2ccc(-c3ccc(N(c4ccc(-c5cccc6oc7ccccc7c56)cc4)c4cccc5c4sc4c(-c6ccccc6)cccc45)cc3)c3ccccc23)c([2H])c1[2H] ; c1ccc(-c2ccc(-c3ccc(N(c4ccc(-c5cccc6oc7ccccc7c56)cc4)c4cccc5c4sc4c(-c6ccccc6)cccc45)cc3)c3ccccc23)cc1 ; c1ccc(-c2cccc3c2sc2c(N(c4ccc(-c5cccc6ccccc56)cc4)c4ccc(-c5cccc6oc7ccccc7c56)cc4)cccc23)cc1 ; c1ccc(-c2cccc3c2sc2c(N(c4ccc(-c5ccc6ccccc6c5)cc4)c4cccc(-c5cccc6oc7ccccc7c56)c4)cccc23)cc1]

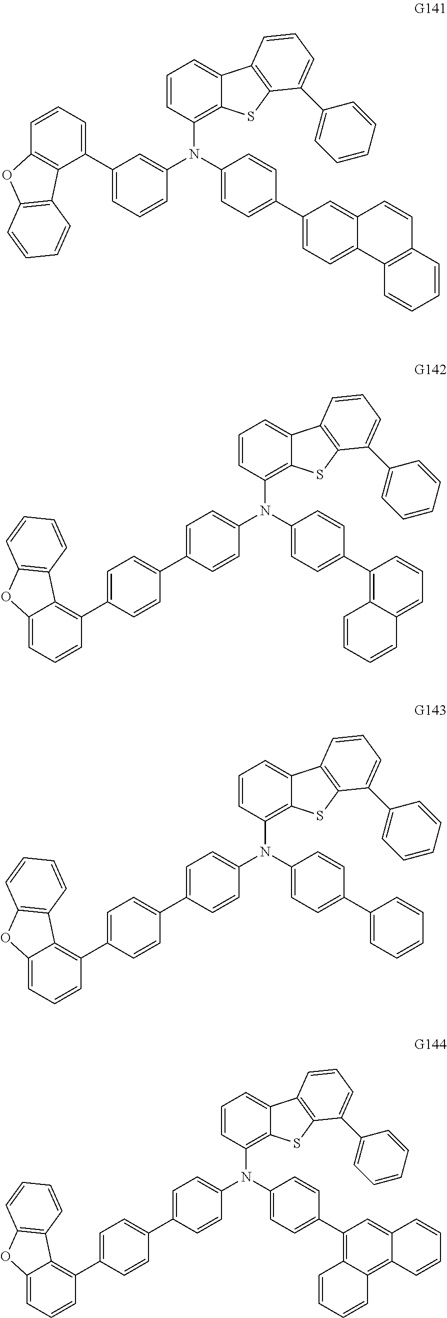 [SMILES: c1ccc(-c2cccc3c2sc2c(N(c4ccc(-c5ccc6c(ccc7ccccc76)c5)cc4)c4cccc(-c5cccc6oc7ccccc7c56)c4)cccc23)cc1 ; c1ccc(-c2cccc3c2sc2c(N(c4ccc(-c5ccc(-c6cccc7oc8ccccc8c67)cc5)cc4)c4ccc(-c5cccc6ccccc56)cc4)cccc23)cc1 ; c1ccc(-c2ccc(N(c3ccc(-c4ccc(-c5cccc6oc7ccccc7c56)cc4)cc3)c3cccc4c3sc3c(-c5ccccc5)cccc34)cc2)cc1 ; c1ccc(-c2cccc3c2sc2c(N(c4ccc(-c5ccc(-c6cccc7oc8ccccc8c67)cc5)cc4)c4ccc(-c5cc6ccccc6c6ccccc56)cc4)cccc23)cc1]

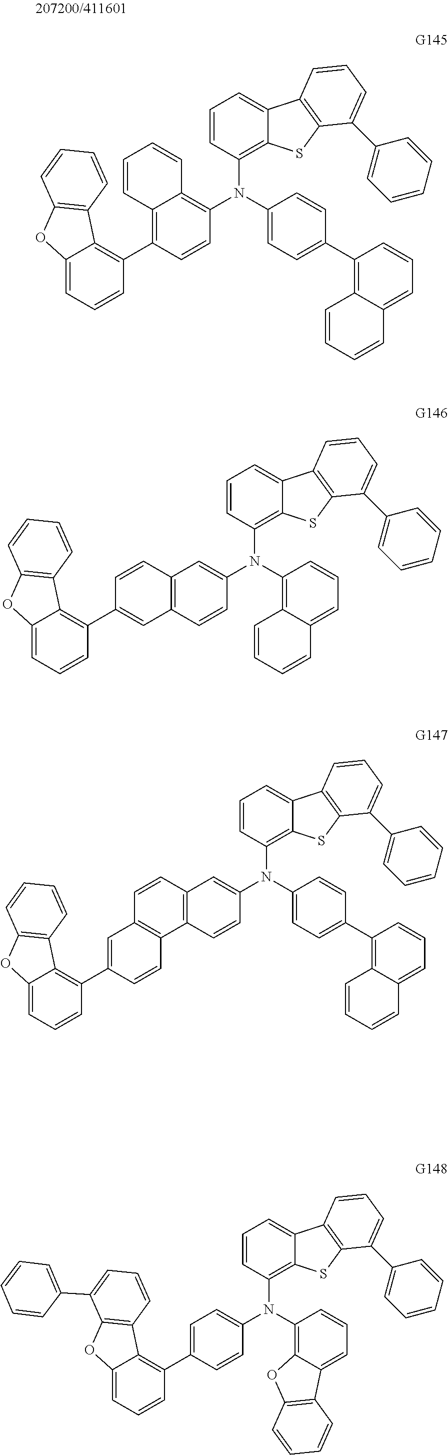 [SMILES: c1ccc(-c2cccc3c2sc2c(N(c4ccc(-c5cccc6ccccc56)cc4)c4ccc(-c5cccc6oc7ccccc7c56)c5ccccc45)cccc23)cc1 ; c1ccc(-c2cccc3c2sc2c(N(c4ccc5cc(-c6cccc7oc8ccccc8c67)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7cccc8oc9ccccc9c78)ccc65)c4)cccc23)cc1 ; c1ccc(-c2cccc3c2oc2cccc(-c4ccc(N(c5cccc6c5oc5ccccc56)c5cccc6c5sc5c(-c7ccccc7)cccc56)cc4)c23)cc1]

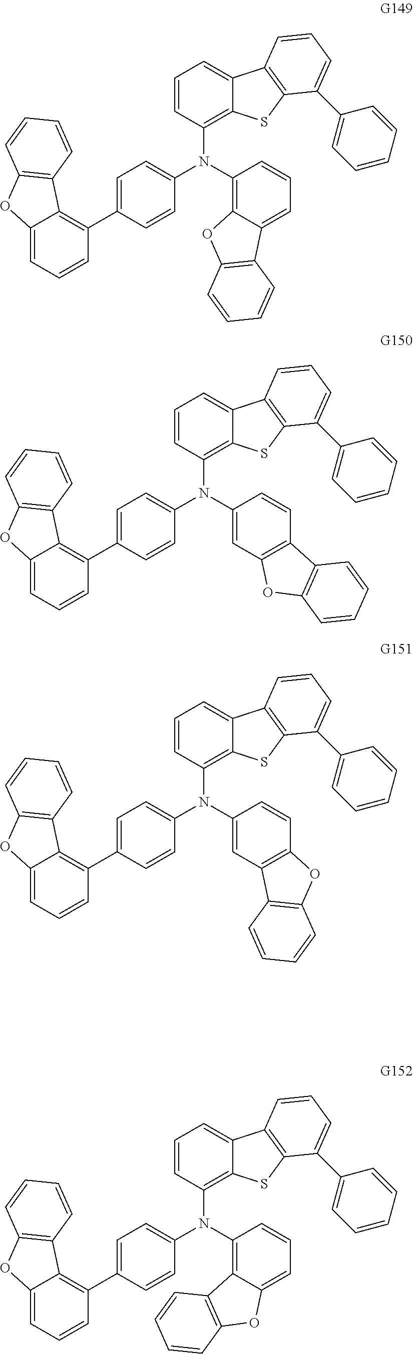 [SMILES: c1ccc(-c2cccc3c2sc2c(N(c4ccc(-c5cccc6oc7ccccc7c56)cc4)c4cccc5c4oc4ccccc45)cccc23)cc1 ; c1ccc(-c2cccc3c2sc2c(N(c4ccc(-c5cccc6oc7ccccc7c56)cc4)c4ccc5c(c4)oc4ccccc45)cccc23)cc1 ; c1ccc(-c2cccc3c2sc2c(N(c4ccc(-c5cccc6oc7ccccc7c56)cc4)c4ccc5oc6ccccc6c5c4)cccc23)cc1 ; c1ccc(-c2cccc3c2sc2c(N(c4ccc(-c5cccc6oc7ccccc7c56)cc4)c4cccc5oc6ccccc6c45)cccc23)cc1]

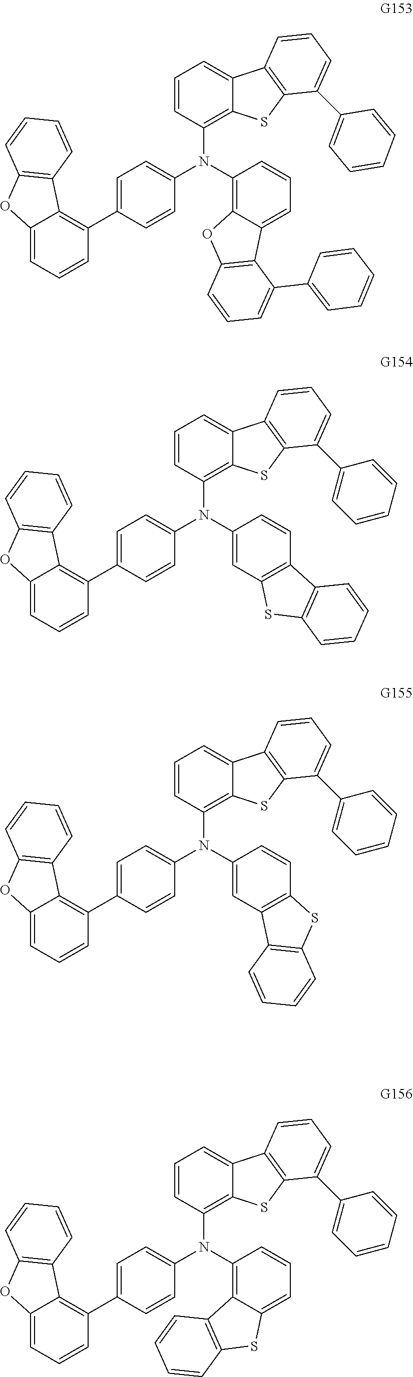 [SMILES: c1ccc(-c2cccc3c2sc2c(N(c4ccc(-c5cccc6oc7ccccc7c56)cc4)c4cccc5c4oc4cccc(-c6ccccc6)c45)cccc23)cc1 ; c1ccc(-c2cccc3c2sc2c(N(c4ccc(-c5cccc6oc7ccccc7c56)cc4)c4ccc5c(c4)sc4ccccc45)cccc23)cc1 ; c1ccc(-c2cccc3c2sc2c(N(c4ccc(-c5cccc6oc7ccccc7c56)cc4)c4ccc5sc6ccccc6c5c4)cccc23)cc1 ; c1ccc(-c2cccc3c2sc2c(N(c4ccc(-c5cccc6oc7ccccc7c56)cc4)c4cccc5sc6ccccc6c45)cccc23)cc1]

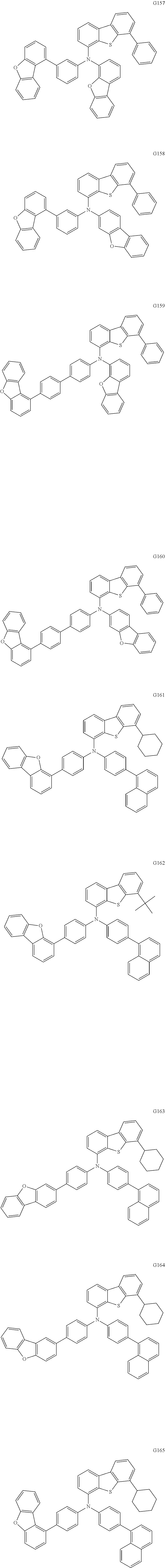

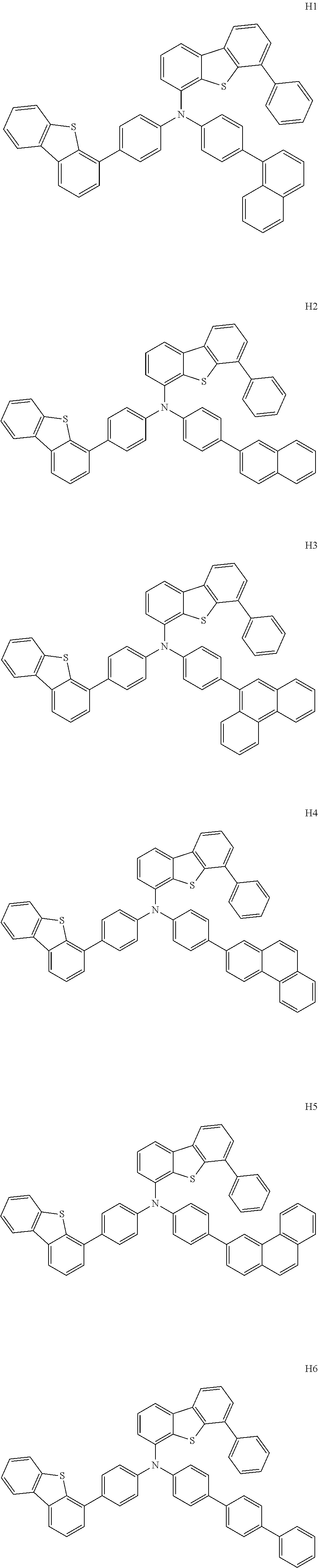

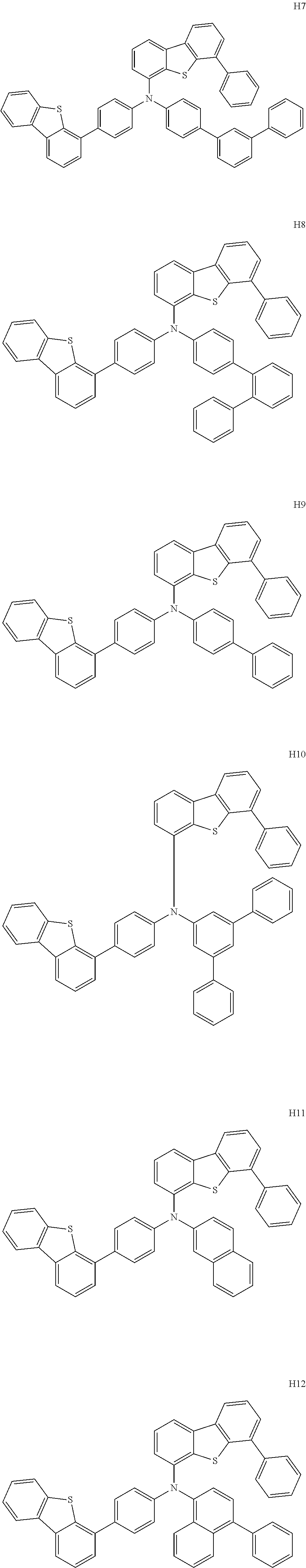

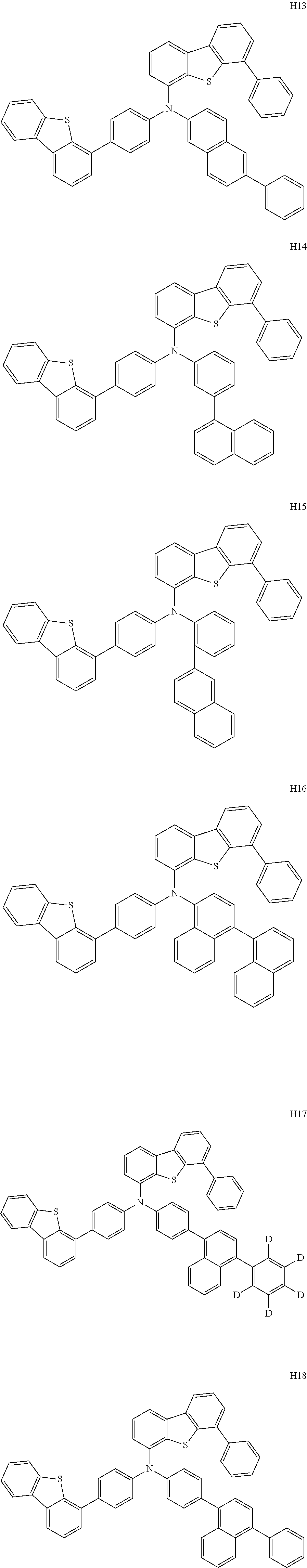

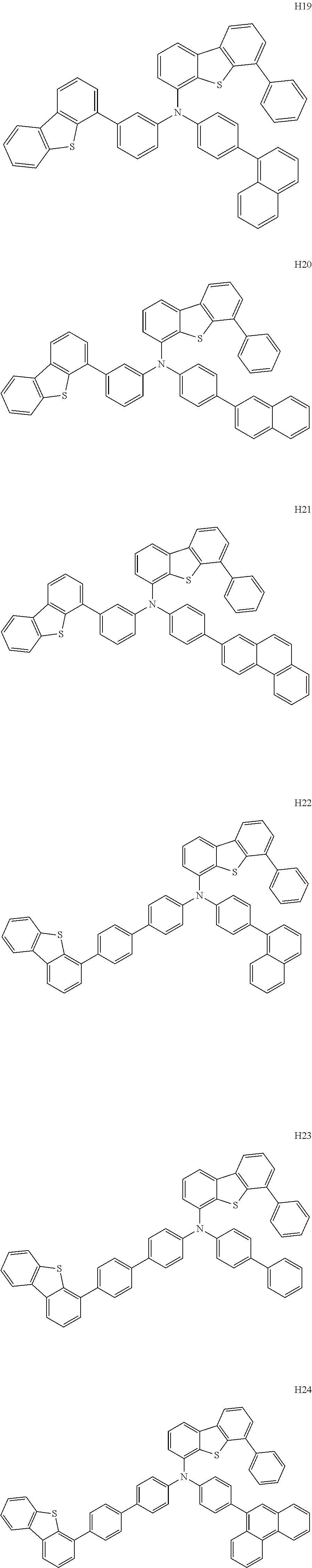

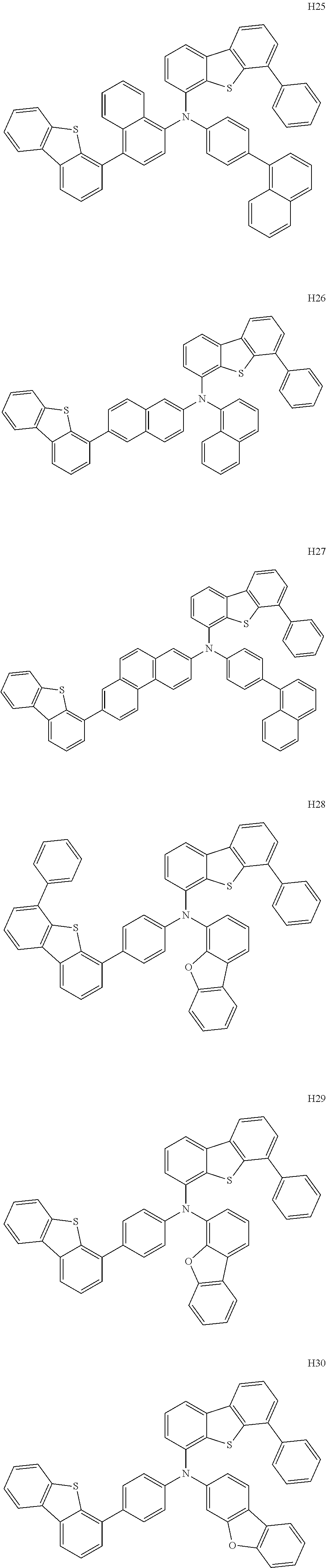

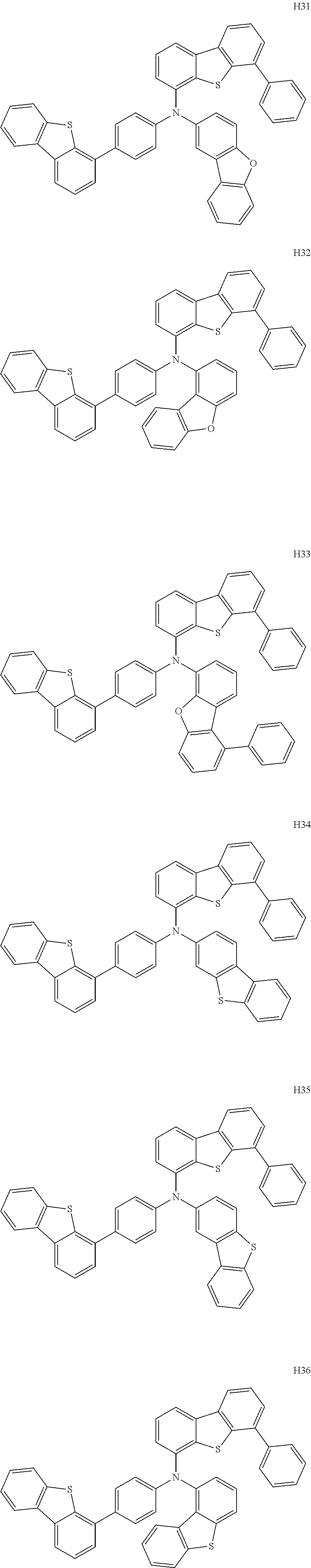

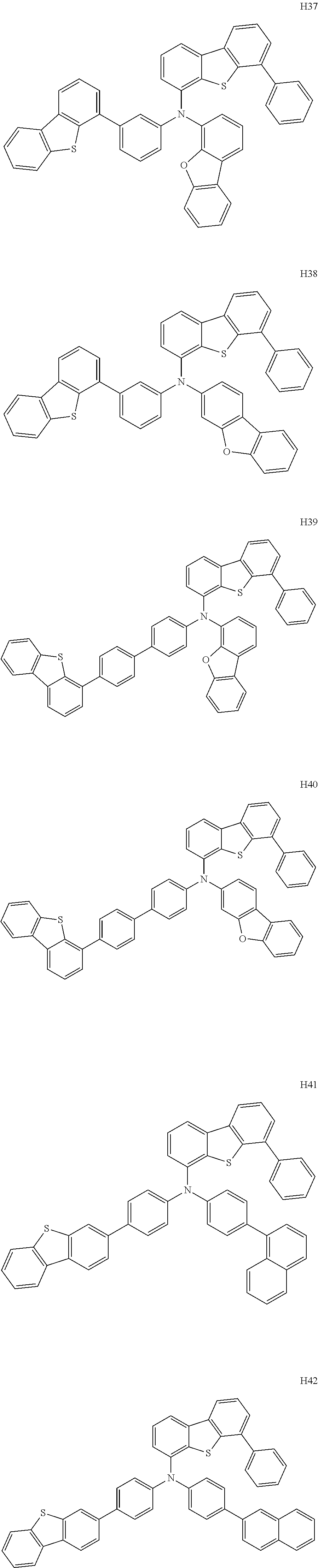

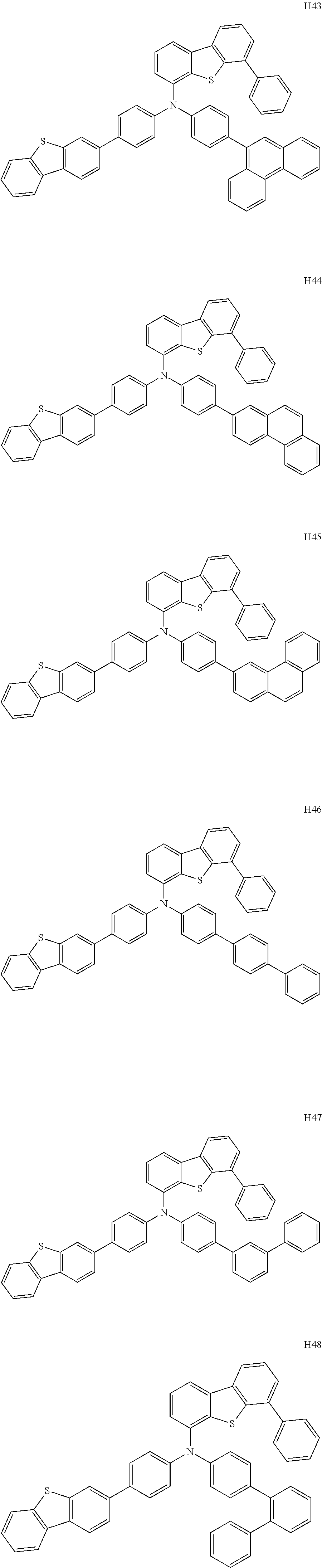

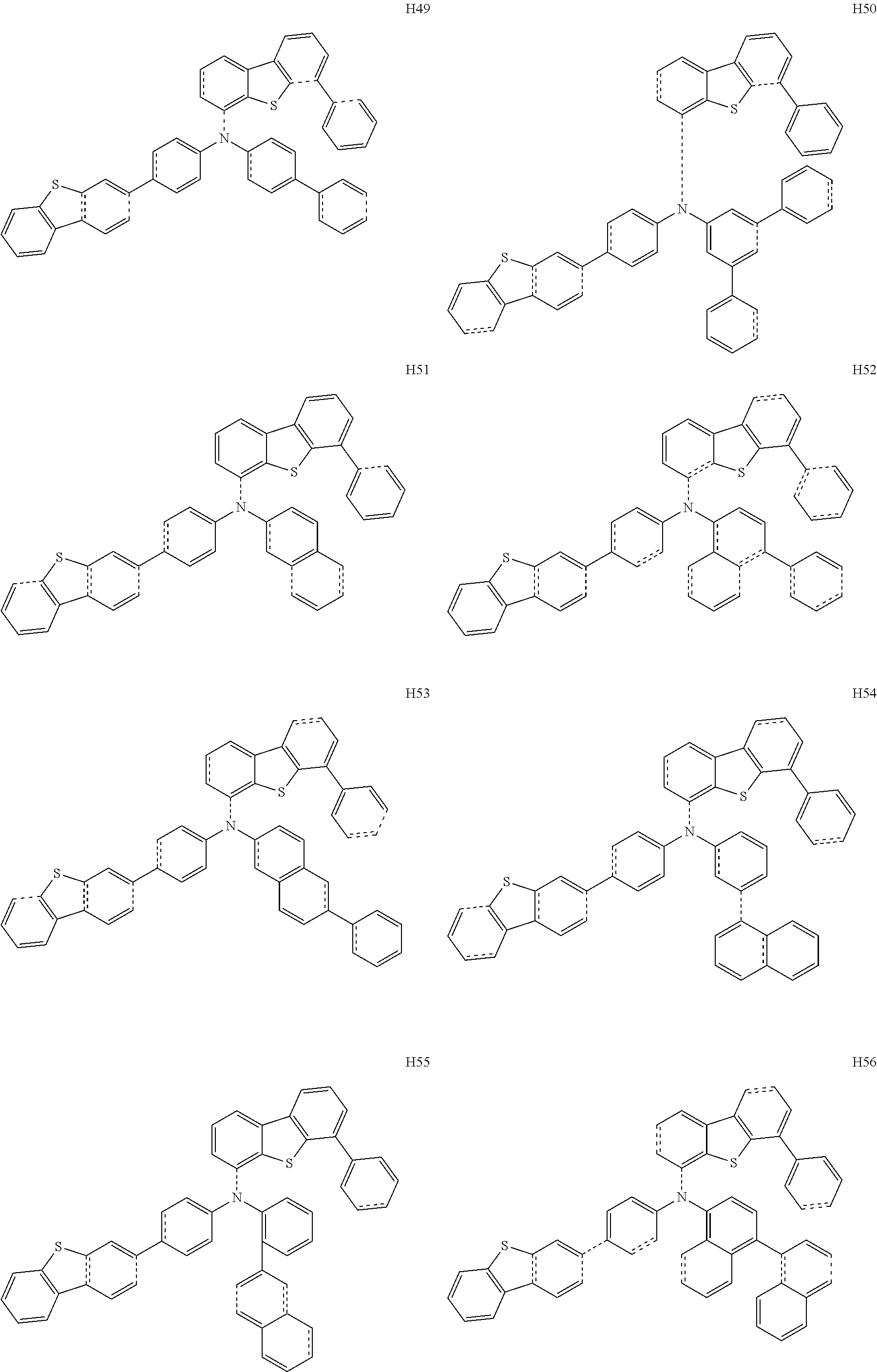

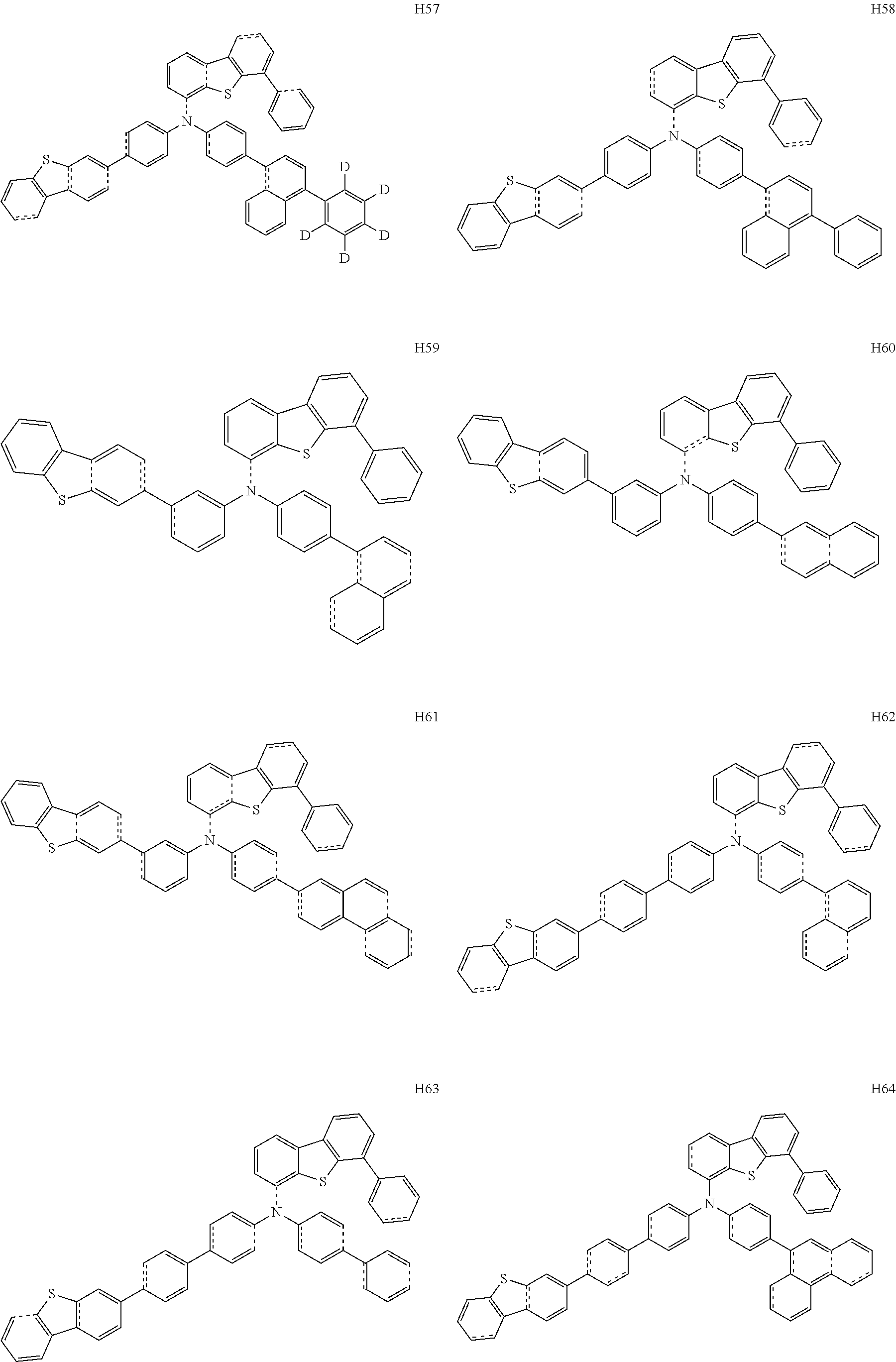

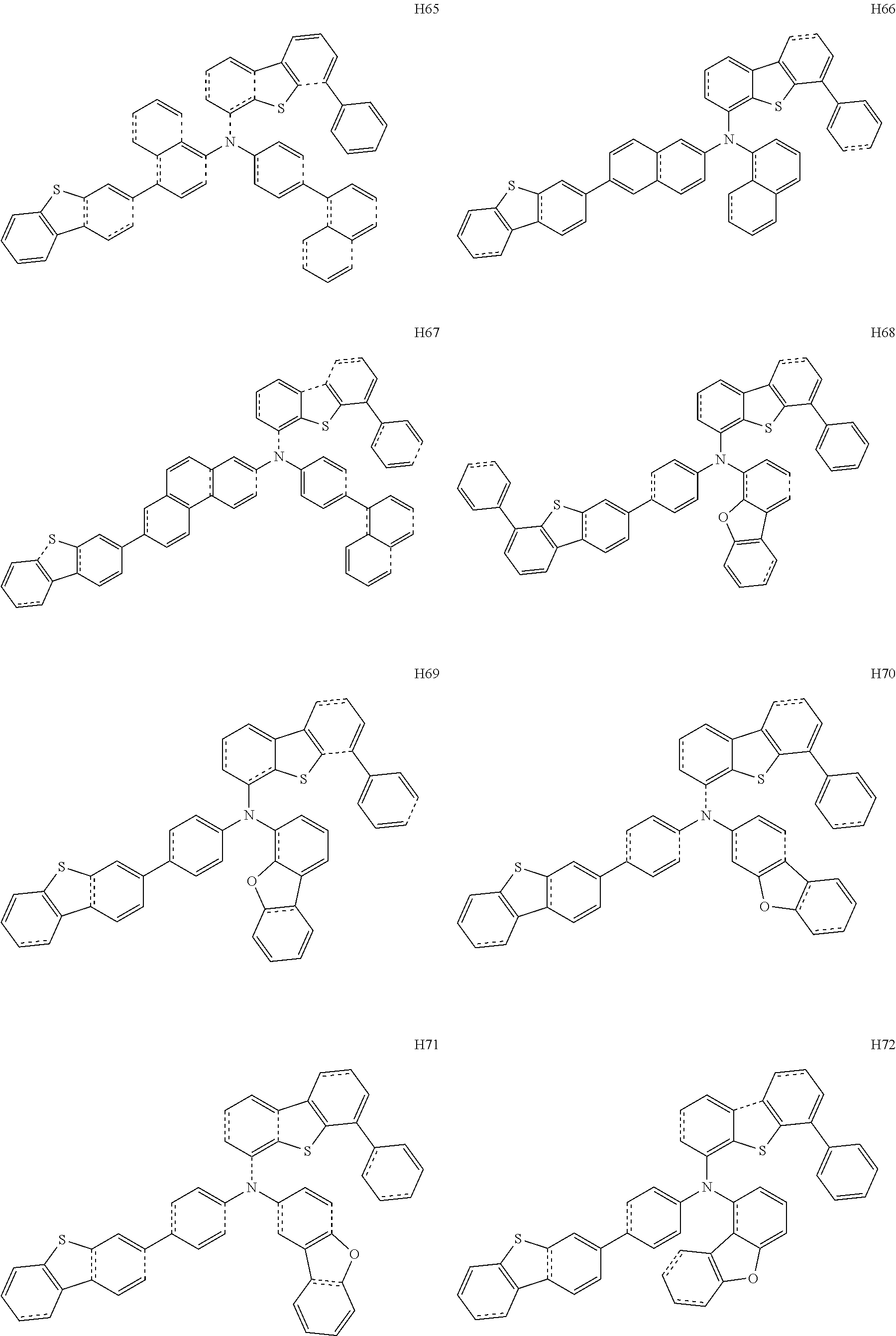

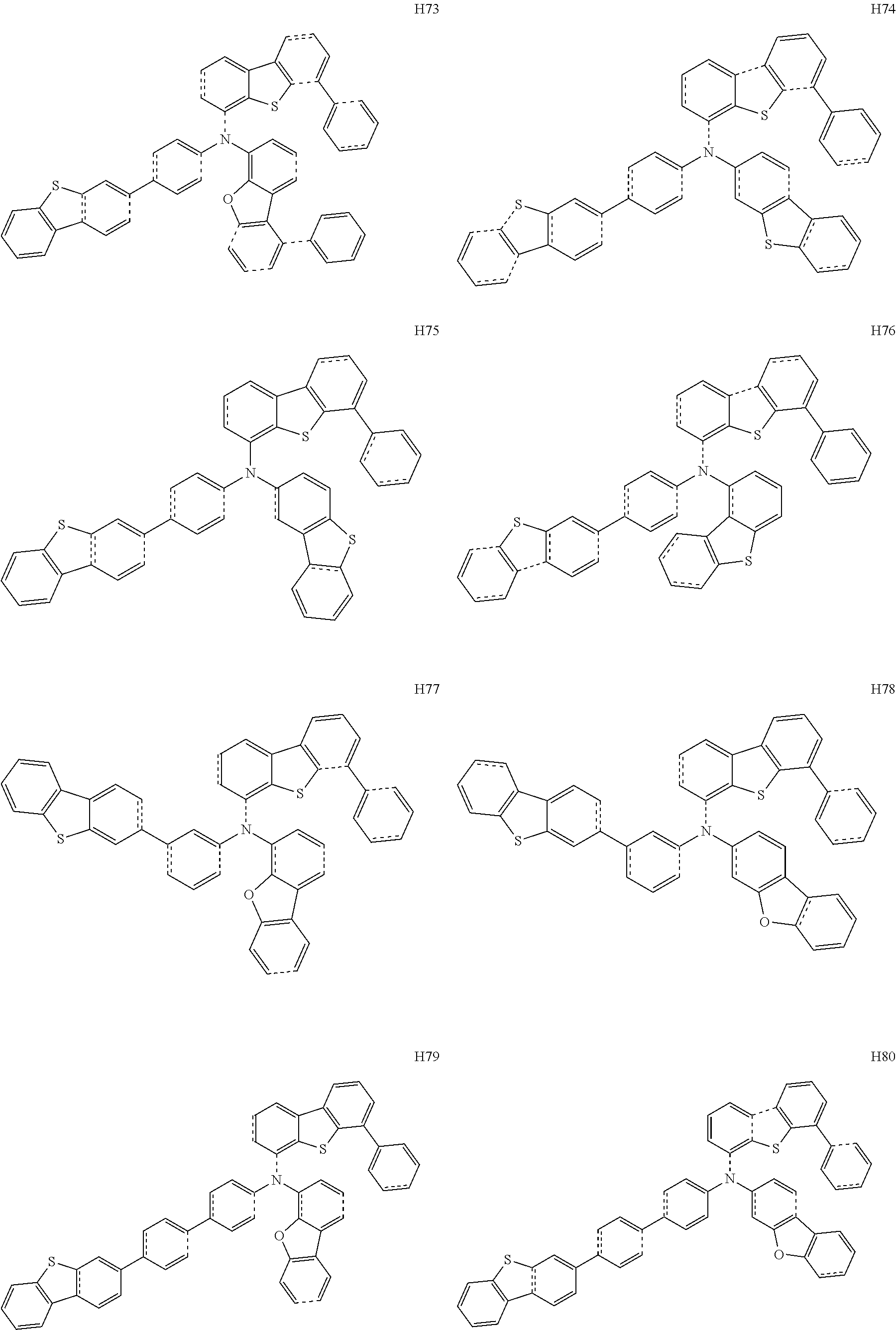

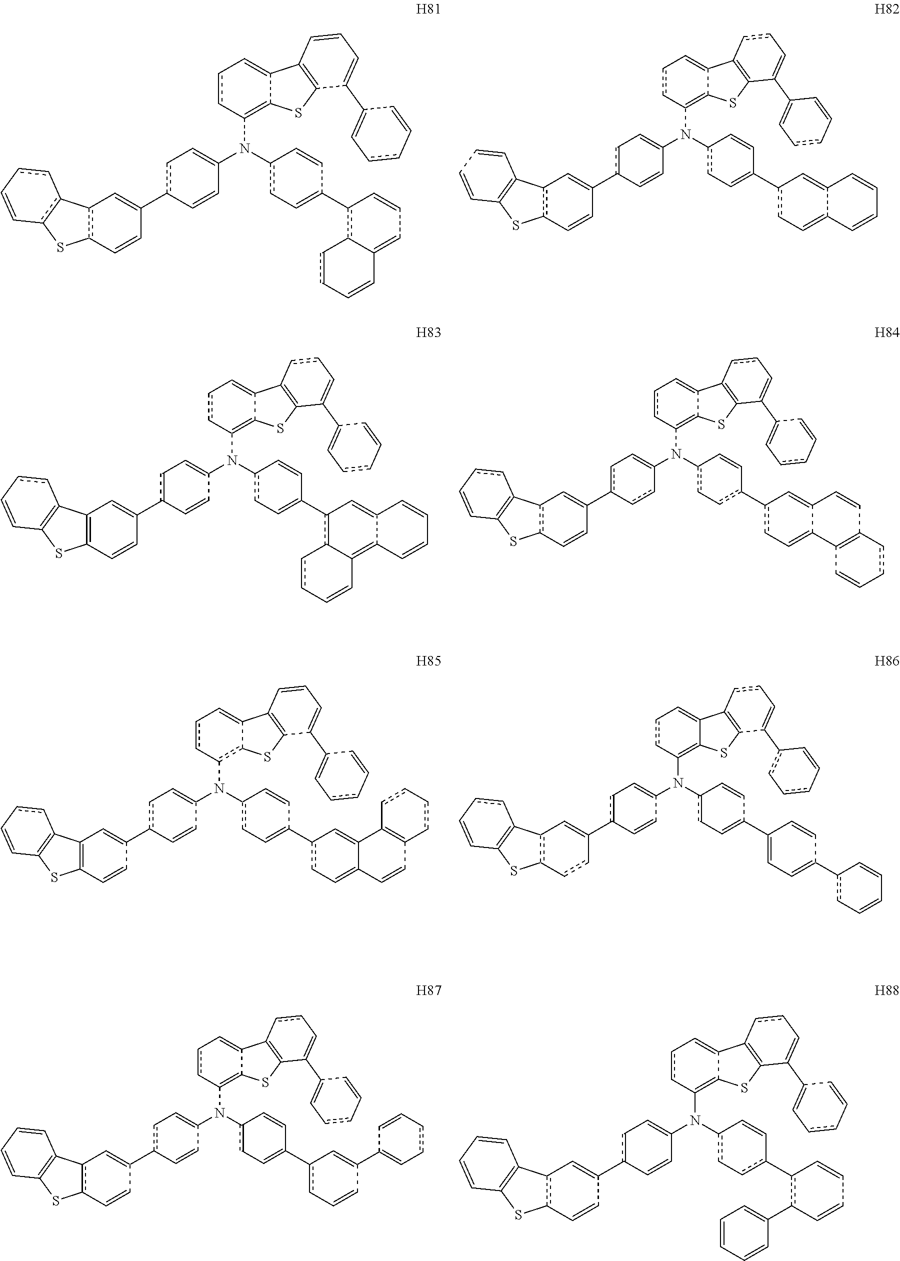

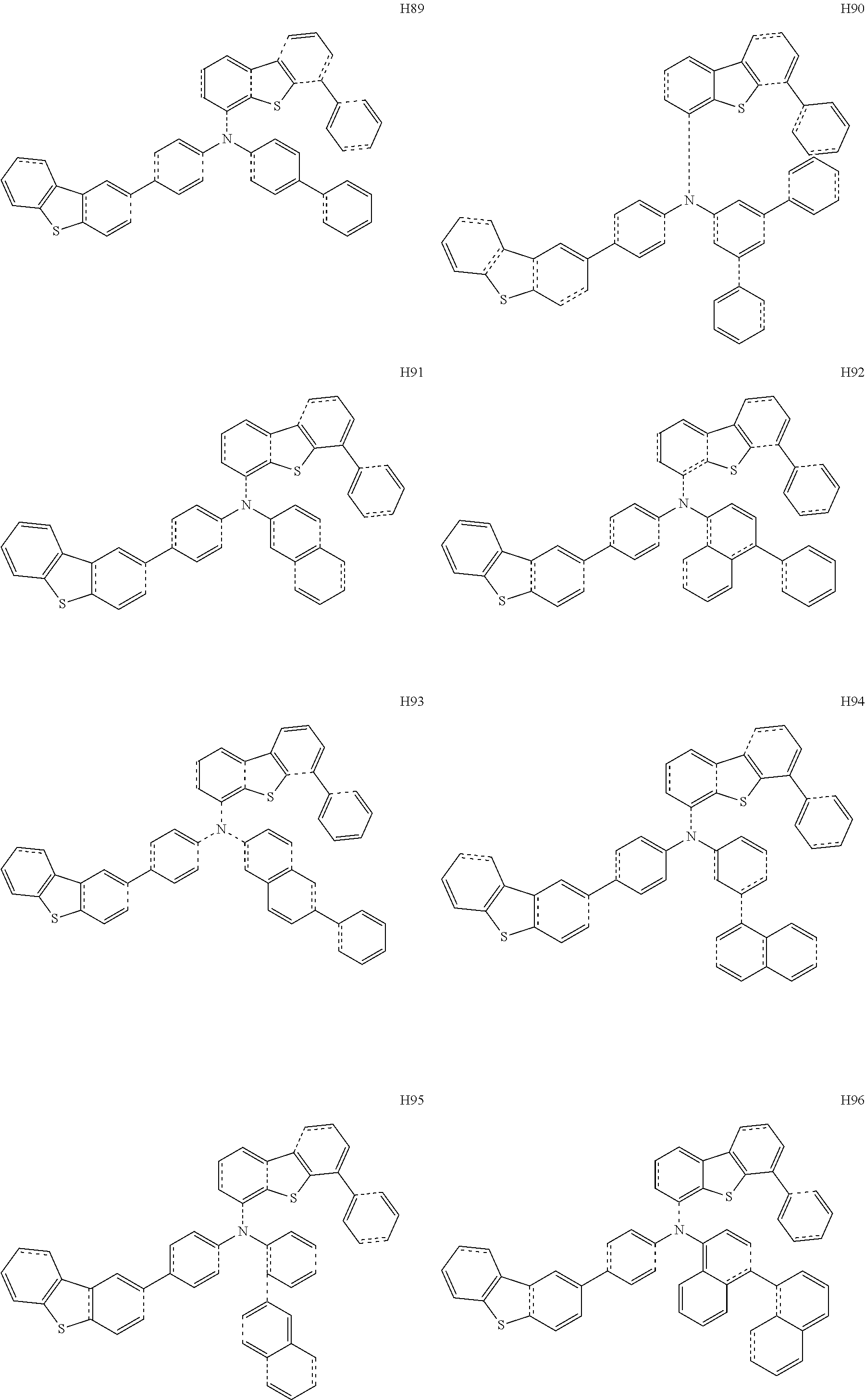

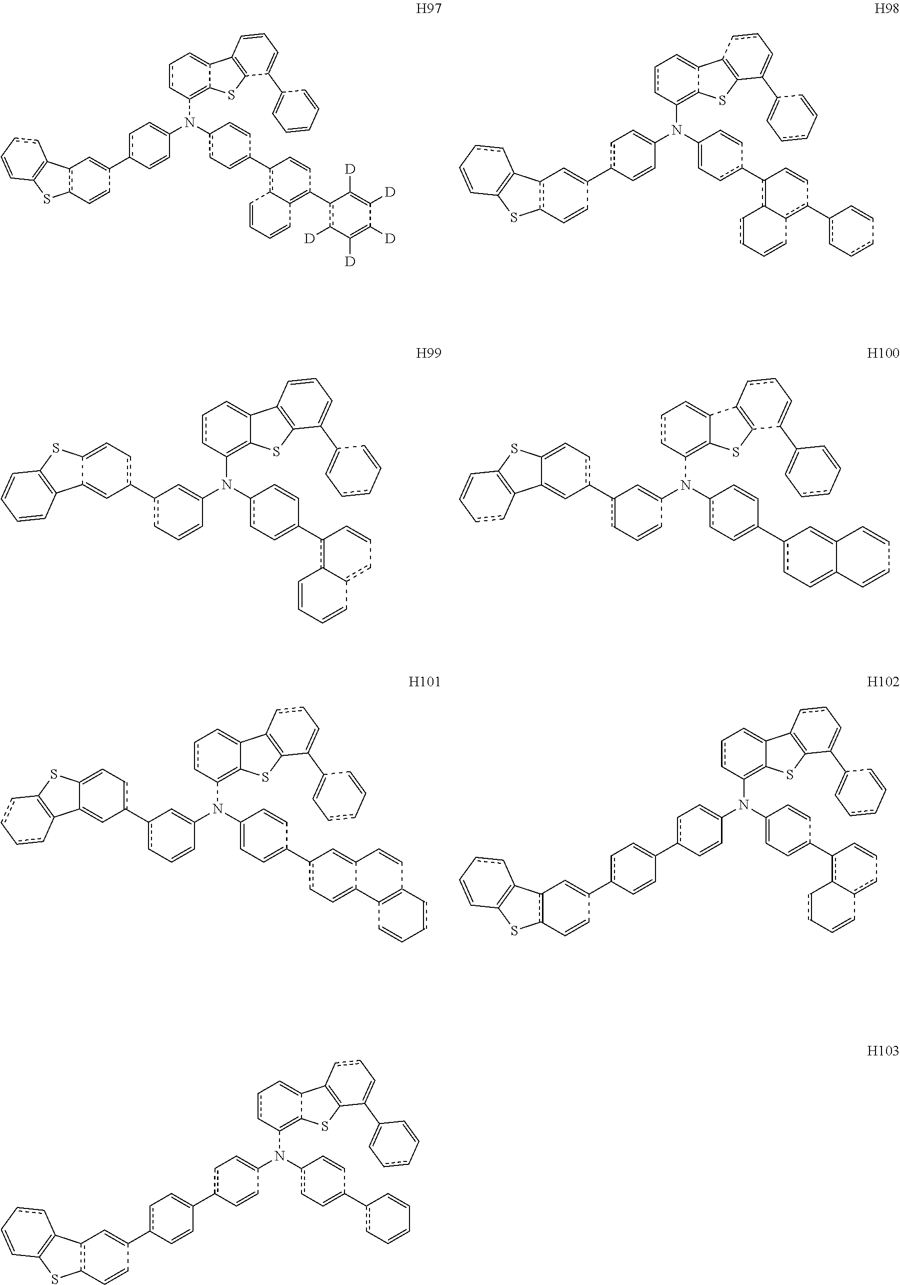

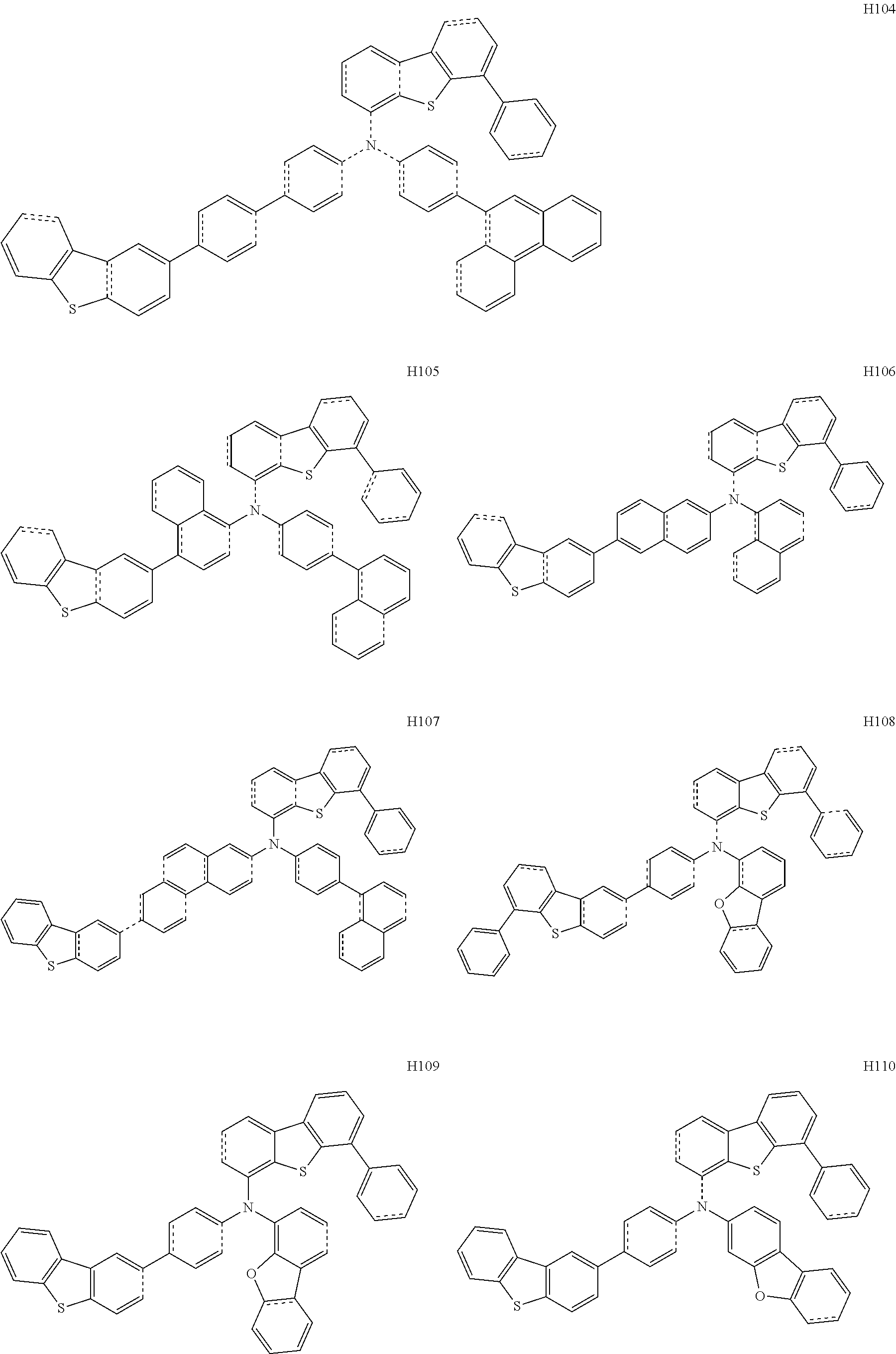

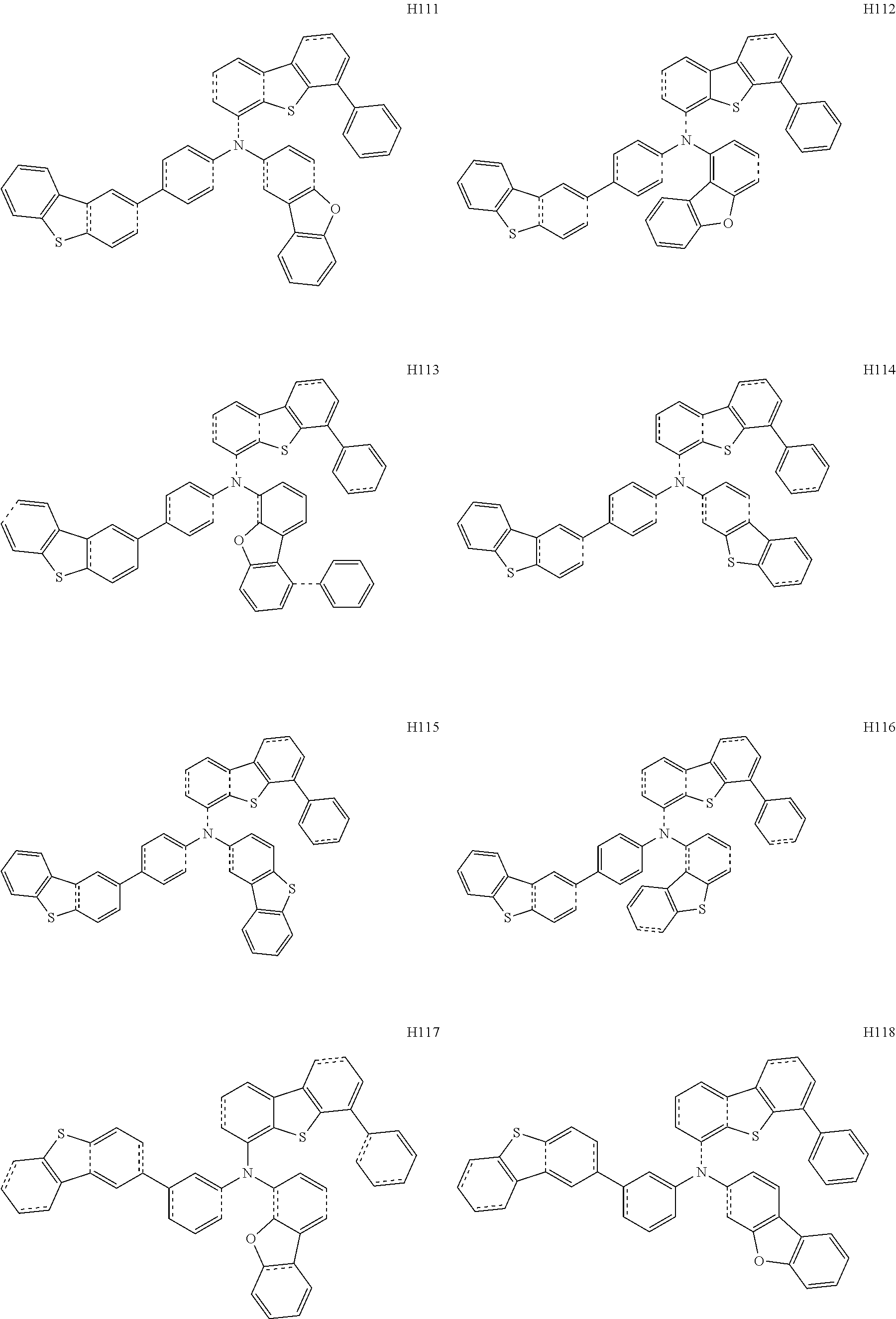

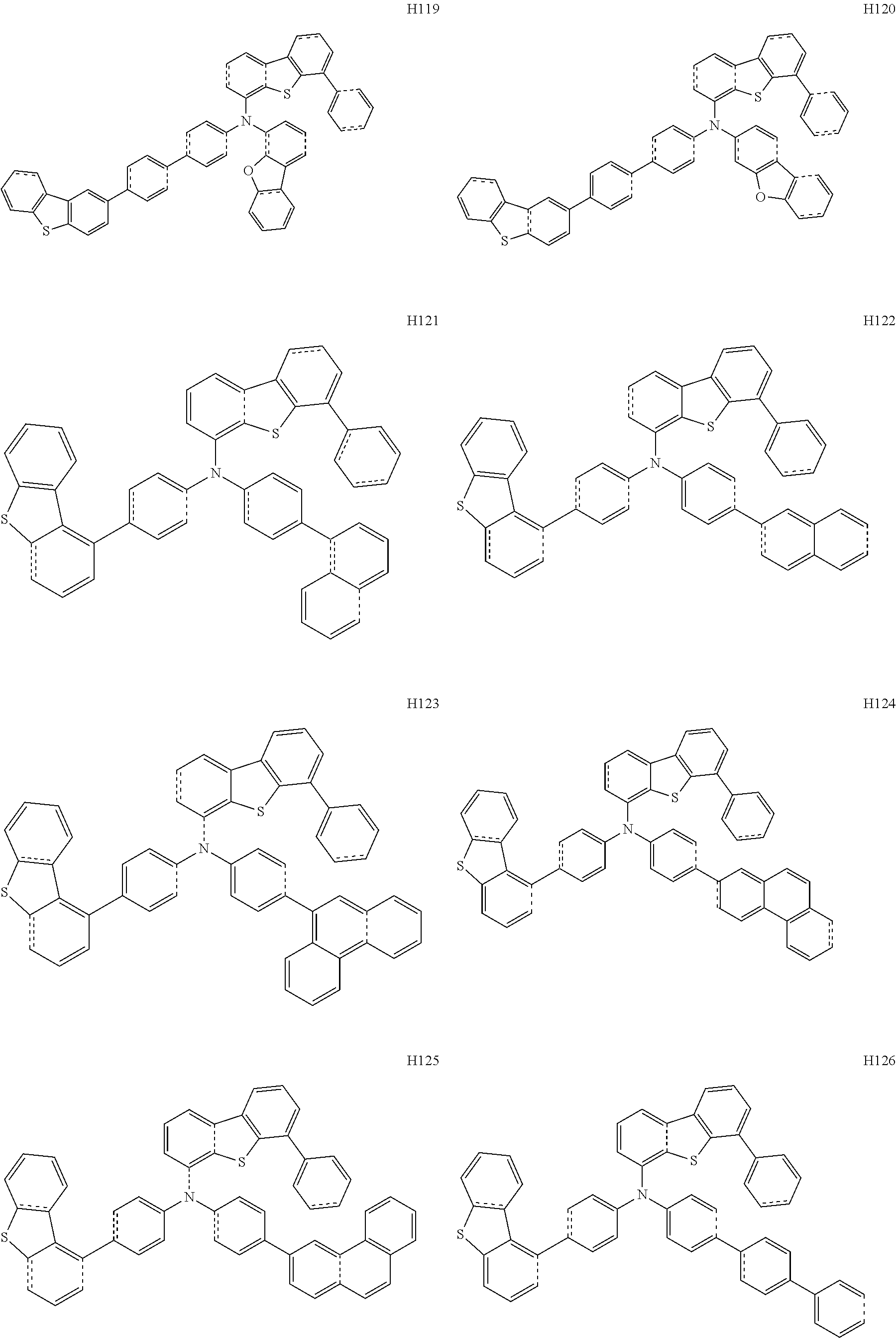

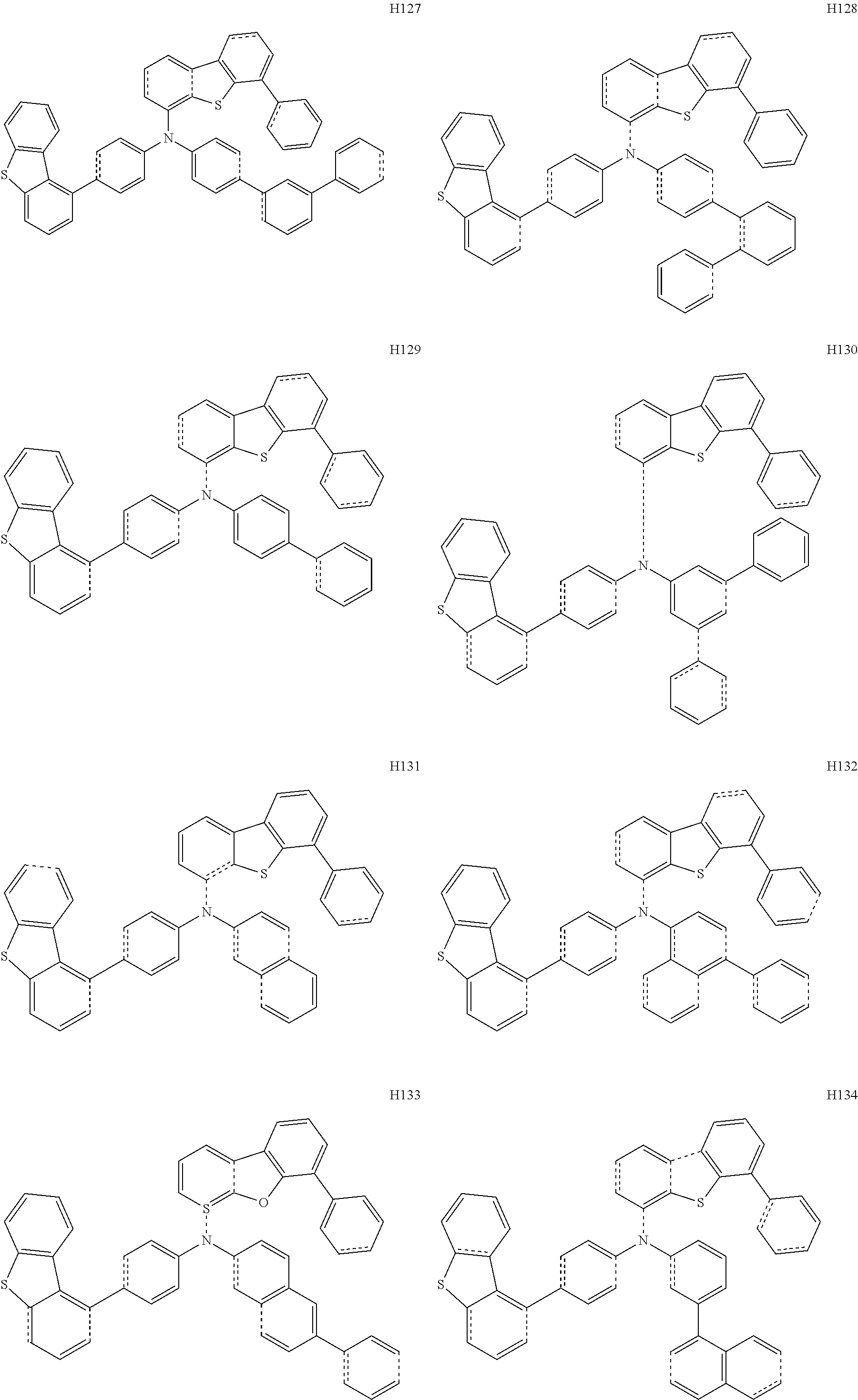

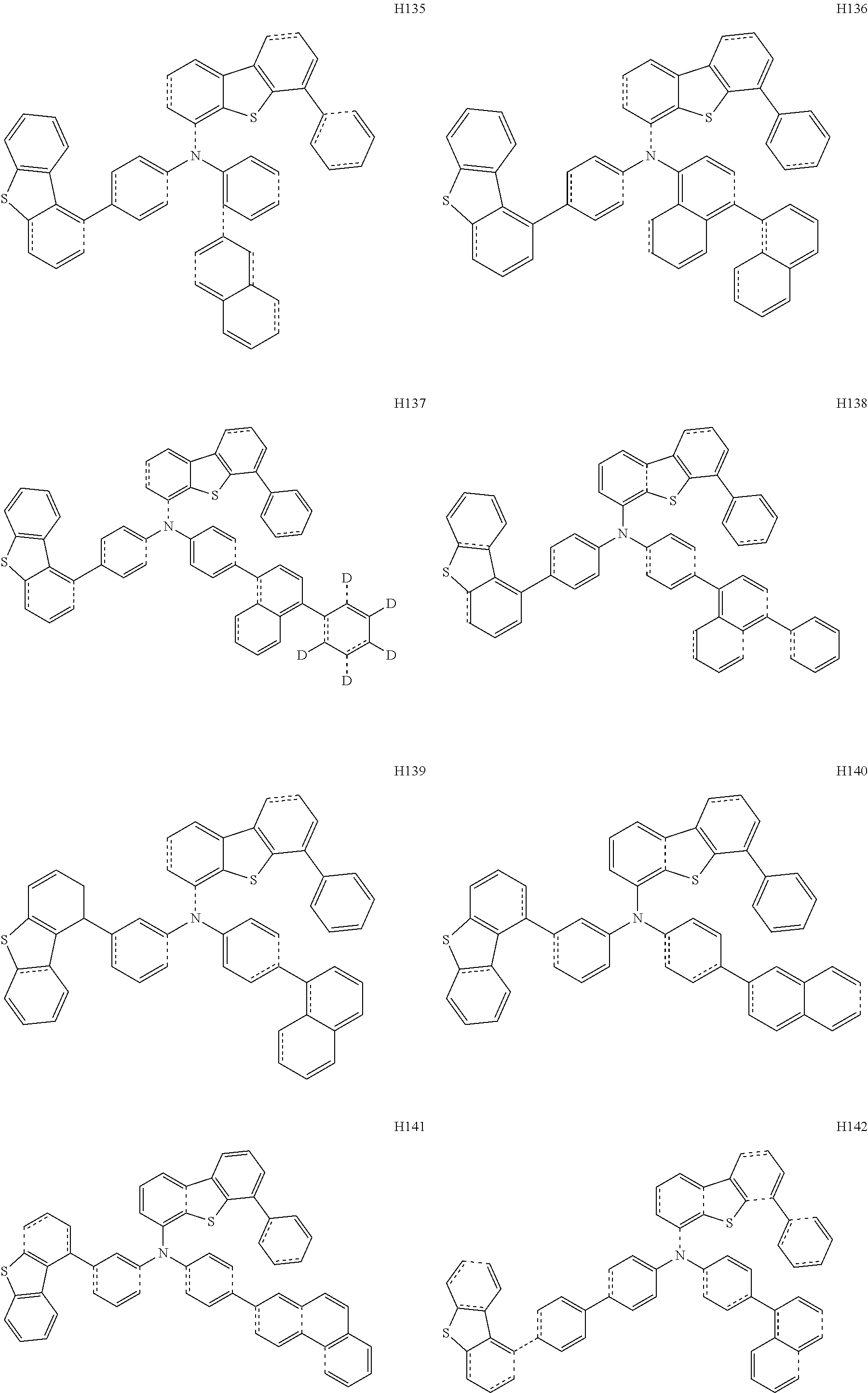

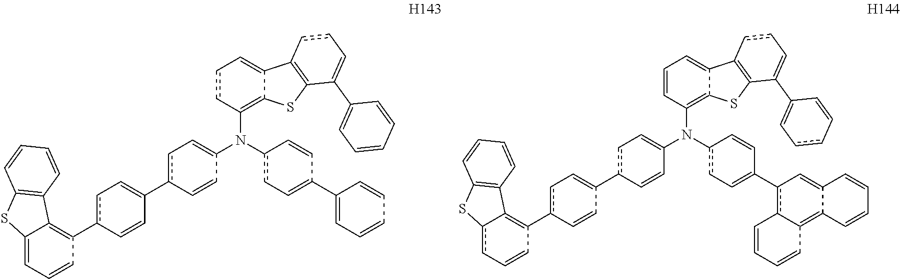

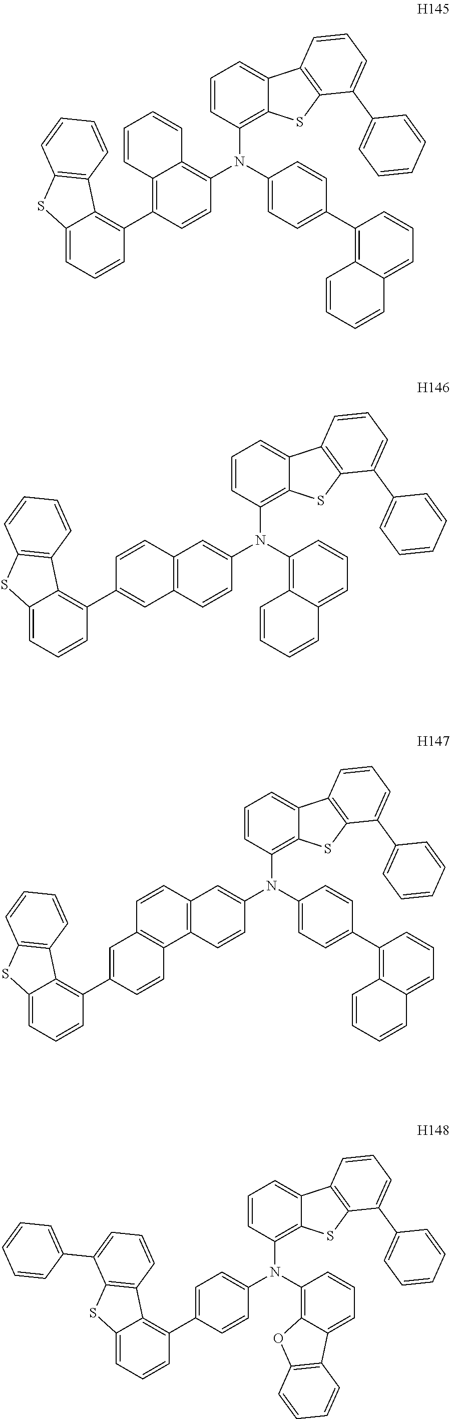 [SMILES: c1ccc(-c2cccc3c2sc2c(N(c4ccc(-c5cccc6ccccc56)cc4)c4ccc(-c5cccc6sc7ccccc7c56)c5ccccc45)cccc23)cc1 ; c1ccc(-c2cccc3c2sc2c(N(c4ccc5cc(-c6cccc7sc8ccccc8c67)ccc5c4)c4cccc5ccccc45)cccc23)cc1 ; c1ccc(-c2cccc3c2sc2c(N(c4ccc(-c5cccc6ccccc56)cc4)c4ccc5c(ccc6cc(-c7cccc8sc9ccccc9c78)ccc65)c4)cccc23)cc1 ; c1ccc(-c2cccc3c2sc2c(N(c4ccc(-c5cccc6sc7c(-c8ccccc8)cccc7c56)cc4)c4cccc5c4oc4ccccc45)cccc23)cc1]

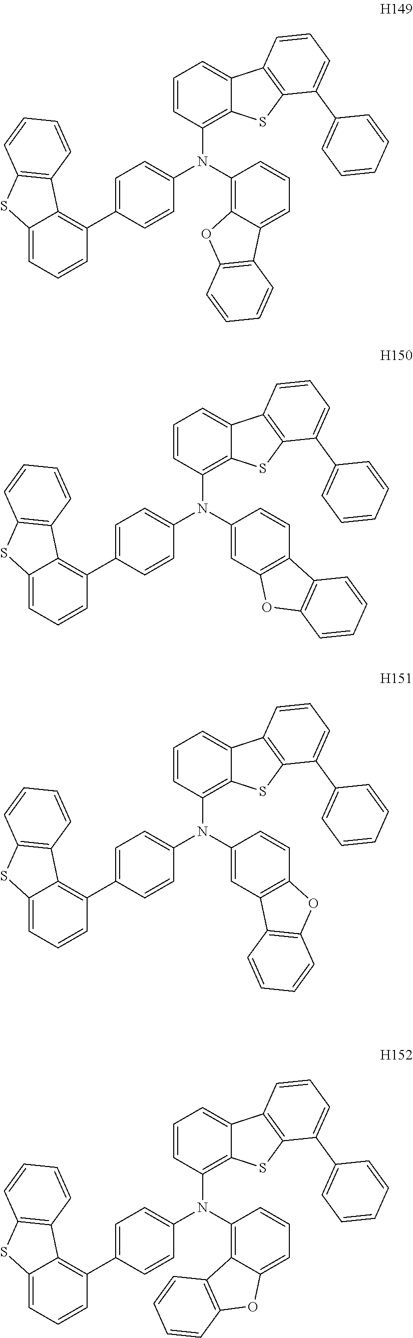 [SMILES: c1ccc(-c2cccc3c2sc2c(N(c4ccc(-c5cccc6sc7ccccc7c56)cc4)c4cccc5c4oc4ccccc45)cccc23)cc1 ; c1ccc(-c2cccc3c2sc2c(N(c4ccc(-c5cccc6sc7ccccc7c56)cc4)c4ccc5c(c4)oc4ccccc45)cccc23)cc1 ; c1ccc(-c2cccc3c2sc2c(N(c4ccc(-c5cccc6sc7ccccc7c56)cc4)c4ccc5oc6ccccc6c5c4)cccc23)cc1 ; c1ccc(-c2cccc3c2sc2c(N(c4ccc(-c5cccc6sc7ccccc7c56)cc4)c4cccc5oc6ccccc6c45)cccc23)cc1]

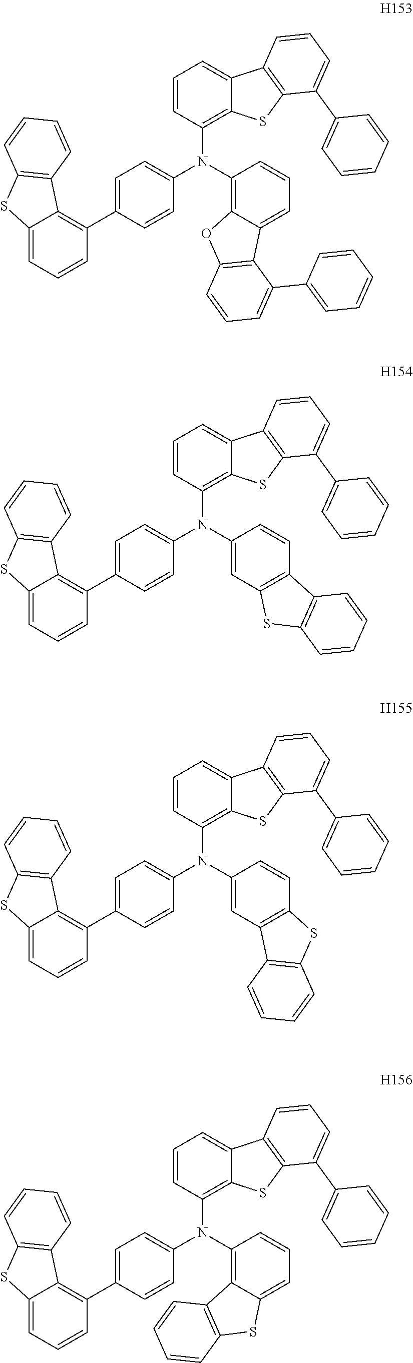 [SMILES: c1ccc(-c2cccc3c2sc2c(N(c4ccc(-c5cccc6sc7ccccc7c56)cc4)c4cccc5c4oc4cccc(-c6ccccc6)c45)cccc23)cc1 ; c1ccc(-c2cccc3c2sc2c(N(c4ccc(-c5cccc6sc7ccccc7c56)cc4)c4ccc5c(c4)sc4ccccc45)cccc23)cc1 ; c1ccc(-c2cccc3c2sc2c(N(c4ccc(-c5cccc6sc7ccccc7c56)cc4)c4ccc5sc6ccccc6c5c4)cccc23)cc1 ; c1ccc(-c2cccc3c2sc2c(N(c4ccc(-c5cccc6sc7ccccc7c56)cc4)c4cccc5sc6ccccc6c45)cccc23)cc1]

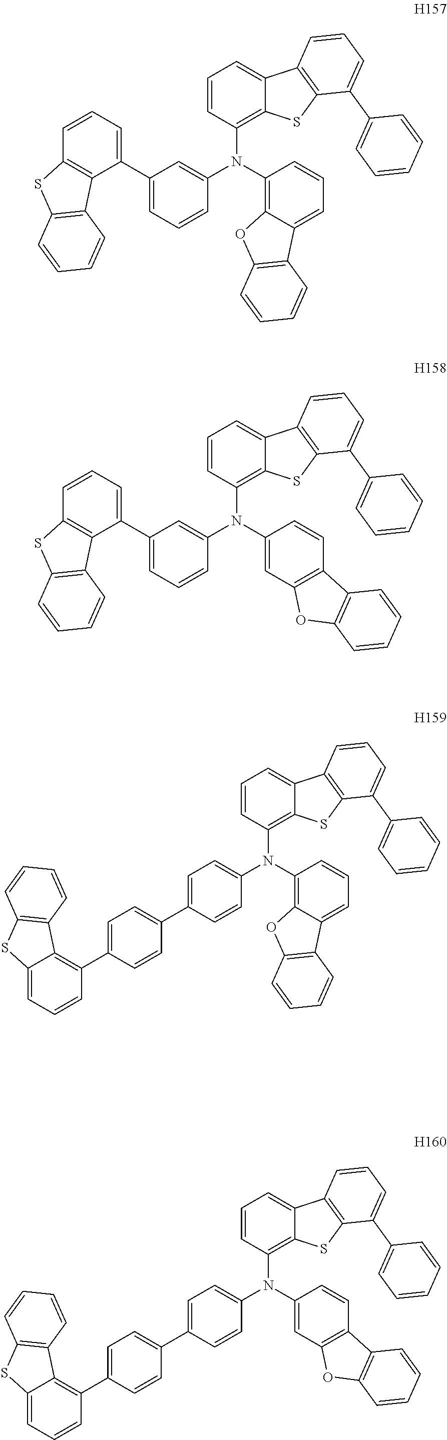 [SMILES: c1ccc(-c2cccc3c2sc2c(N(c4cccc(-c5cccc6sc7ccccc7c56)c4)c4cccc5c4oc4ccccc45)cccc23)cc1 ; c1ccc(-c2cccc3c2sc2c(N(c4cccc(-c5cccc6sc7ccccc7c56)c4)c4ccc5c(c4)oc4ccccc45)cccc23)cc1 ; c1ccc(-c2cccc3c2sc2c(N(c4ccc(-c5ccc(-c6cccc7sc8ccccc8c67)cc5)cc4)c4cccc5c4oc4ccccc45)cccc23)cc1 ; c1ccc(-c2cccc3c2sc2c(N(c4ccc(-c5ccc(-c6cccc7sc8ccccc8c67)cc5)cc4)c4ccc5c(c4)oc4ccccc45)cccc23)cc1]

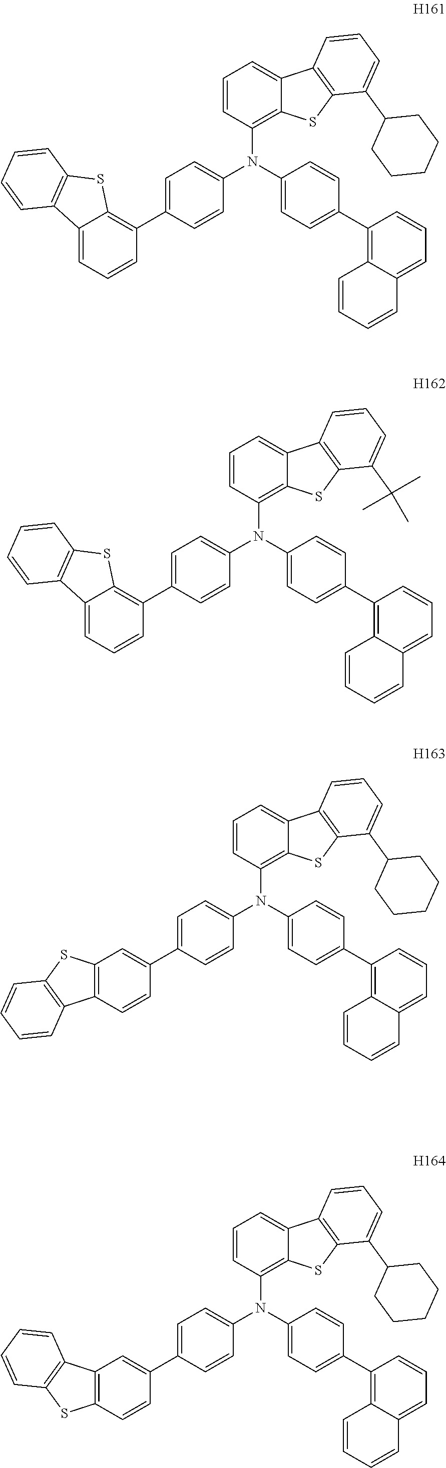 [SMILES: c1ccc2c(-c3ccc(N(c4ccc(-c5cccc6c5sc5ccccc56)cc4)c4cccc5c4sc4c(C6CCCCC6)cccc45)cc3)cccc2c1 ; CC(C)(C)c1cccc2c1sc1c(N(c3ccc(-c4cccc5ccccc45)cc3)c3ccc(-c4cccc5c4sc4ccccc45)cc3)cccc12 ; c1ccc2c(-c3ccc(N(c4ccc(-c5ccc6c(c5)sc5ccccc56)cc4)c4cccc5c4sc4c(C6CCCCC6)cccc45)cc3)cccc2c1 ; c1ccc2c(-c3ccc(N(c4ccc(-c5ccc6sc7ccccc7c6c5)cc4)c4cccc5c4sc4c(C6CCCCC6)cccc45)cc3)cccc2c1]

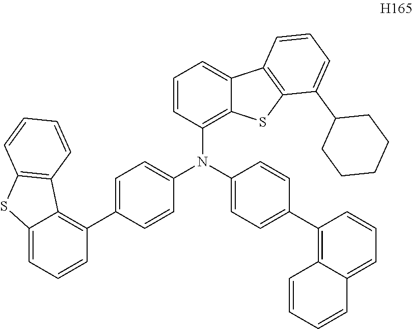 [SMILES: c1ccc2c(-c3ccc(N(c4ccc(-c5cccc6sc7ccccc7c56)cc4)c4cccc5c4sc4c(C6CCCCC6)cccc45)cc3)cccc2c1]

20. The amine compound of claim 15, wherein the amine compound represented by Formula 1 is any one selected from among compounds represented in Compound Group 2:

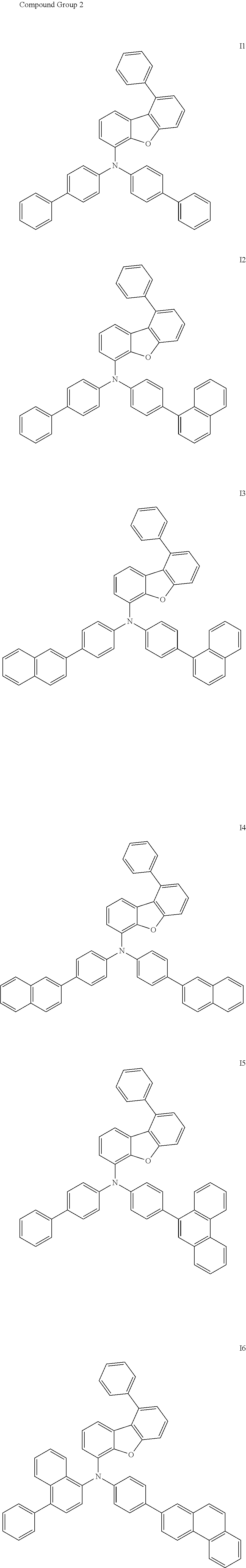

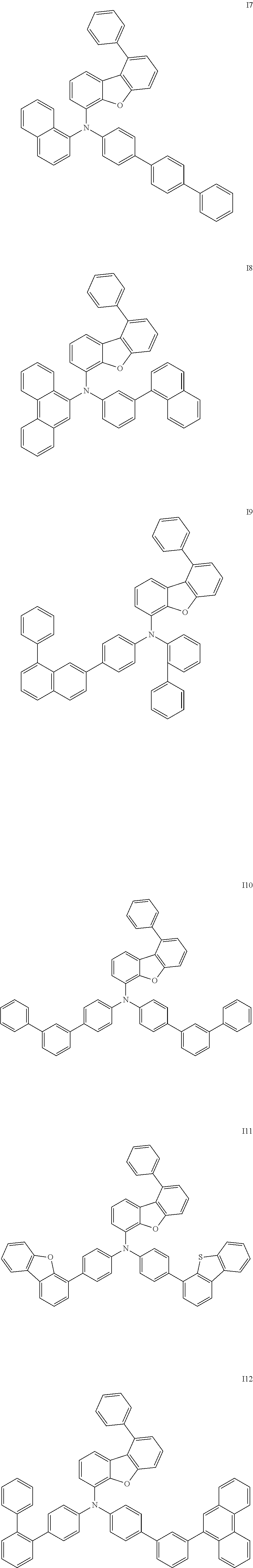

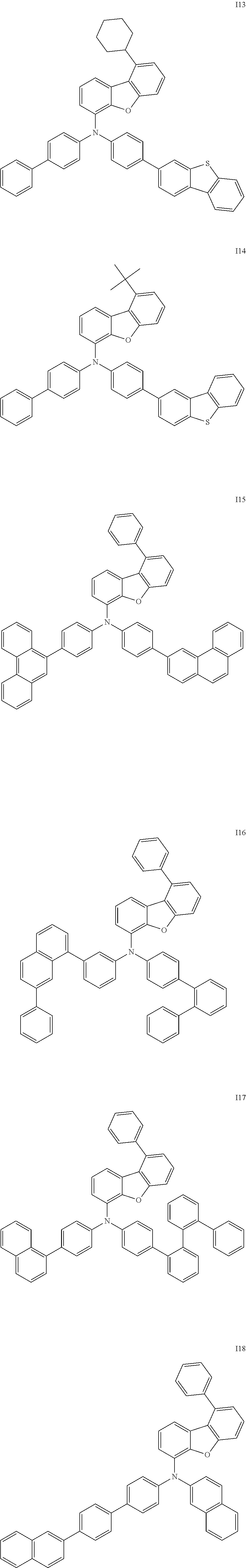

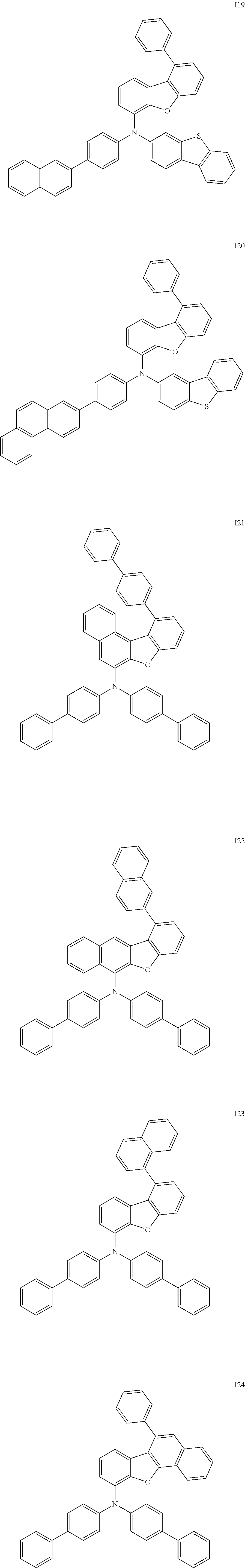

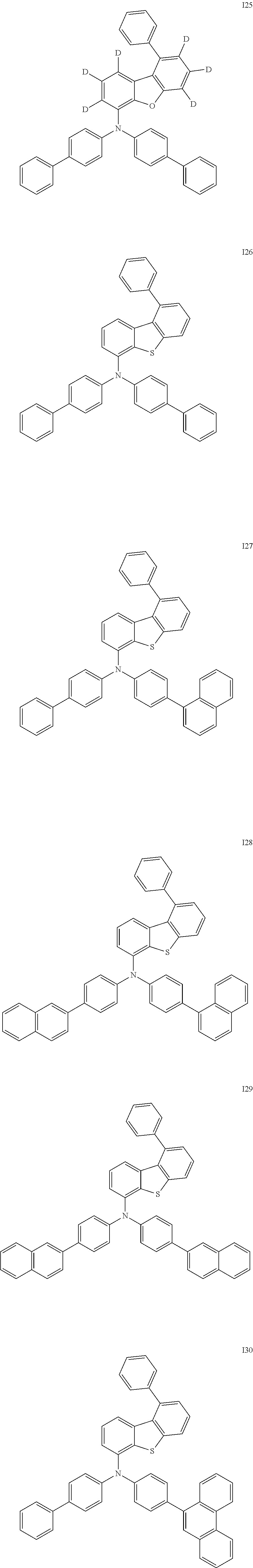

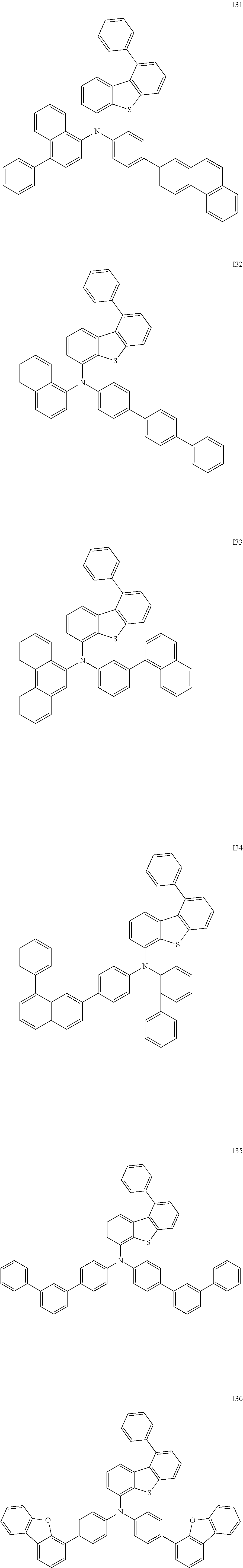

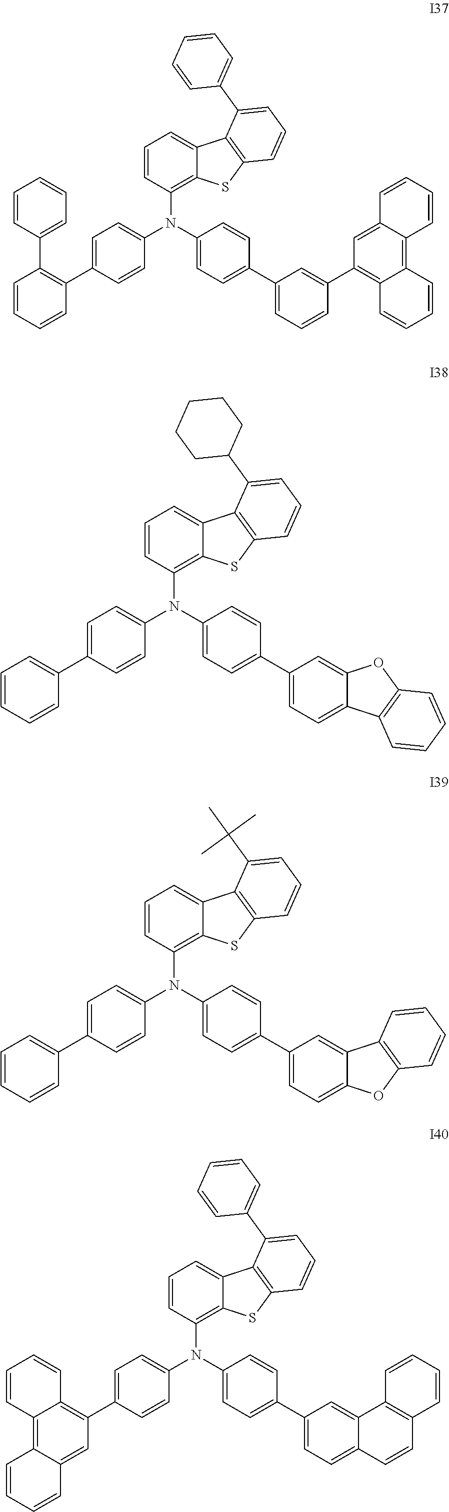 [SMILES: c1ccc(-c2ccccc2-c2ccc(N(c3ccc(-c4cccc(-c5cc6ccccc6c6ccccc56)c4)cc3)c3cccc4c3sc3cccc(-c5ccccc5)c34)cc2)cc1 ; c1ccc(-c2ccc(N(c3ccc(-c4ccc5c(c4)oc4ccccc45)cc3)c3cccc4c3sc3cccc(C5CCCCC5)c34)cc2)cc1 ; CC(C)(C)c1cccc2sc3c(N(c4ccc(-c5ccccc5)cc4)c4ccc(-c5ccc6oc7ccccc7c6c5)cc4)cccc3c12 ; c1ccc(-c2cccc3sc4c(N(c5ccc(-c6ccc7ccc8ccccc8c7c6)cc5)c5ccc(-c6cc7ccccc7c7ccccc67)cc5)cccc4c23)cc1]

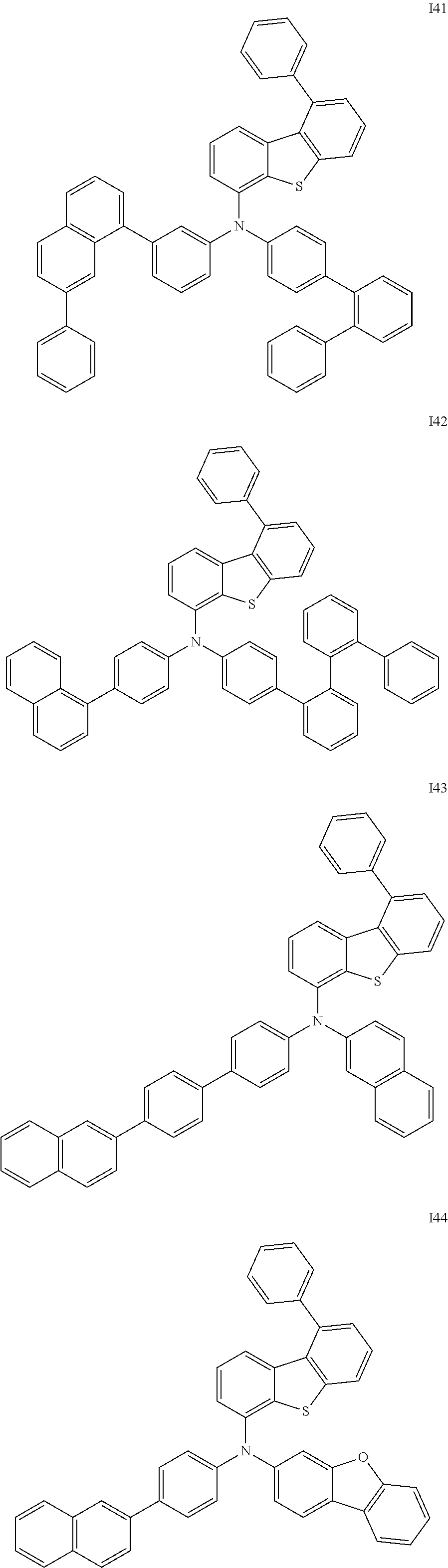 [SMILES: c1ccc(-c2ccc3cccc(-c4cccc(N(c5ccc(-c6ccccc6-c6ccccc6)cc5)c5cccc6c5sc5cccc(-c7ccccc7)c56)c4)c3c2)cc1 ; c1ccc(-c2ccccc2-c2ccccc2-c2ccc(N(c3ccc(-c4cccc5ccccc45)cc3)c3cccc4c3sc3cccc(-c5ccccc5)c34)cc2)cc1 ; c1ccc(-c2cccc3sc4c(N(c5ccc(-c6ccc(-c7ccc8ccccc8c7)cc6)cc5)c5ccc6ccccc6c5)cccc4c23)cc1 ; c1ccc(-c2cccc3sc4c(N(c5ccc(-c6ccc7ccccc7c6)cc5)c5ccc6c(c5)oc5ccccc56)cccc4c23)cc1]

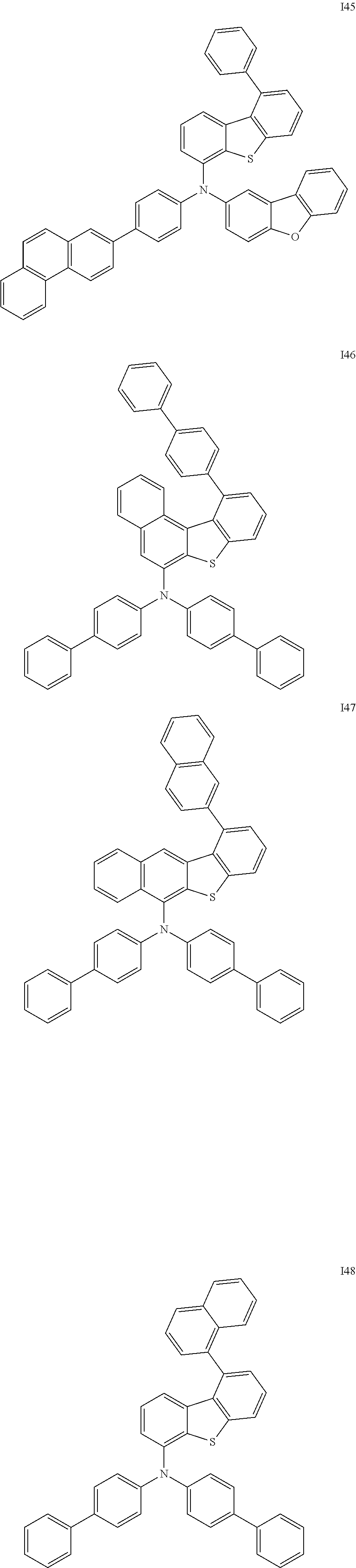 [SMILES: c1ccc(-c2cccc3sc4c(N(c5ccc(-c6ccc7c(ccc8ccccc87)c6)cc5)c5ccc6oc7ccccc7c6c5)cccc4c23)cc1 ; c1ccc(-c2ccc(-c3cccc4sc5c(N(c6ccc(-c7ccccc7)cc6)c6ccc(-c7ccccc7)cc6)cc6ccccc6c5c34)cc2)cc1 ; c1ccc(-c2ccc(N(c3ccc(-c4ccccc4)cc3)c3c4ccccc4cc4c3sc3cccc(-c5ccc6ccccc6c5)c34)cc2)cc1 ; c1ccc(-c2ccc(N(c3ccc(-c4ccccc4)cc3)c3cccc4c3sc3cccc(-c5cccc6ccccc56)c34)cc2)cc1]

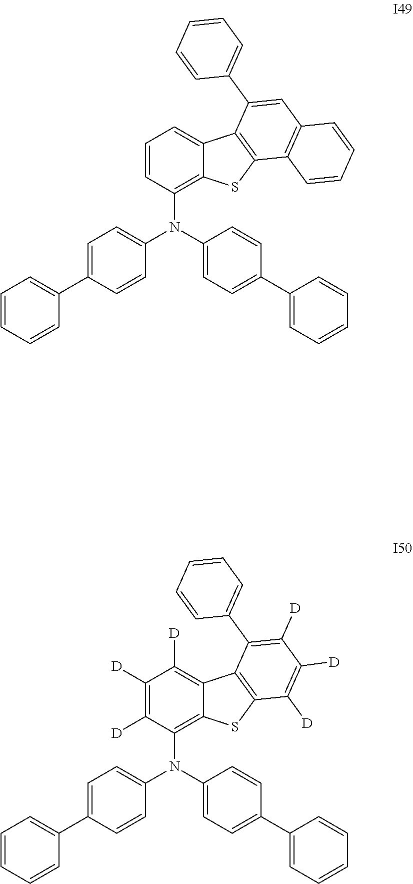 [SMILES: c1ccc(-c2ccc(N(c3ccc(-c4ccccc4)cc3)c3cccc4c3sc3c5ccccc5cc(-c5ccccc5)c43)cc2)cc1 ; [2H]c1c([2H])c(-c2ccccc2)c2c(sc3c(N(c4ccc(-c5ccccc5)cc4)c4ccc(-c5ccccc5)cc4)c([2H])c([2H])c([2H])c32)c1[2H]]

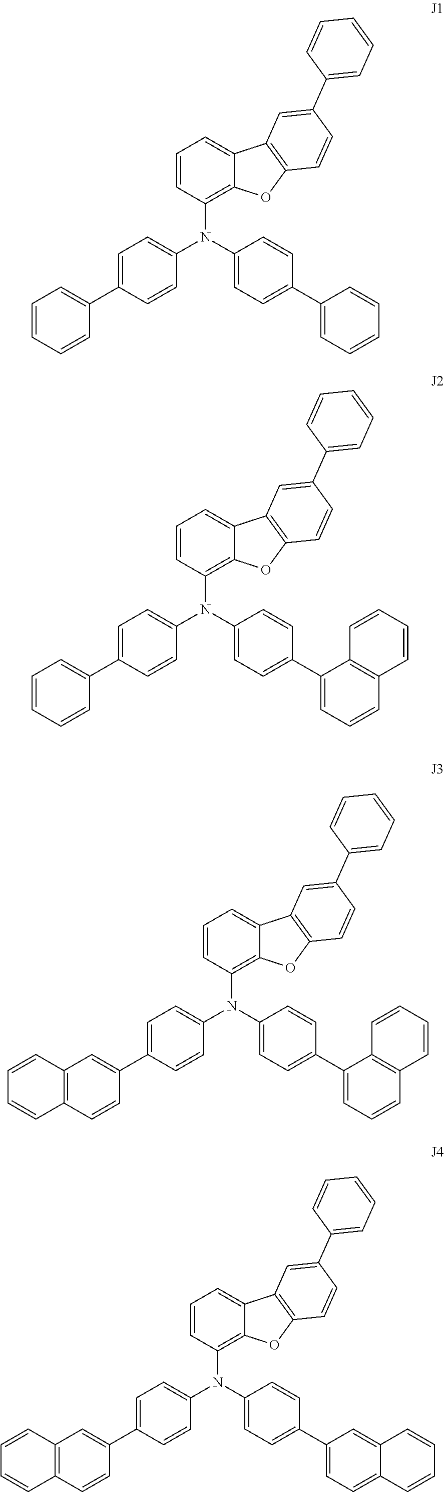

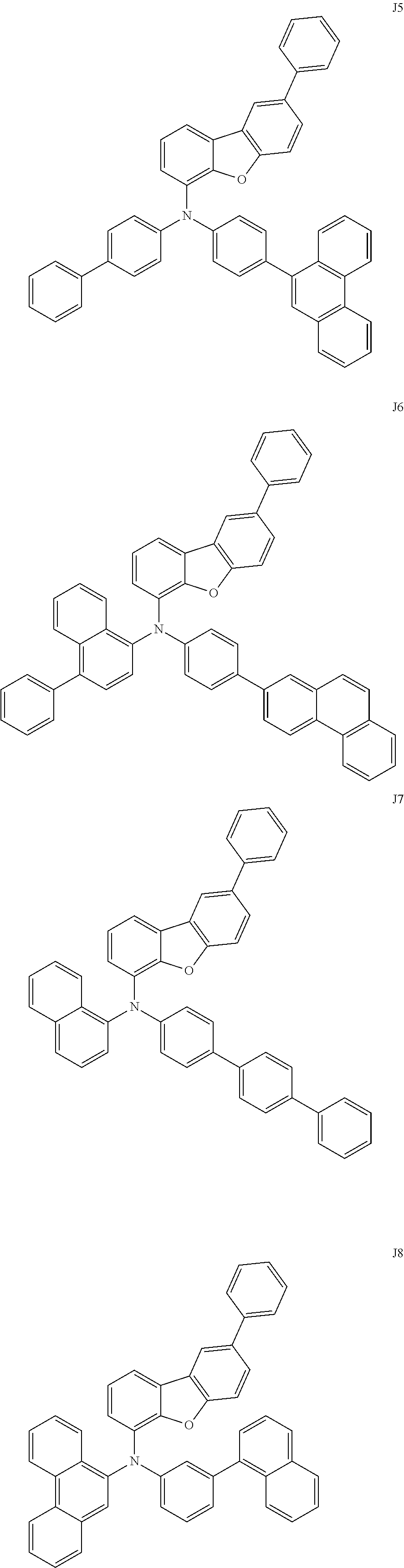 [SMILES: c1ccc(-c2ccc(N(c3ccc(-c4cc5ccccc5c5ccccc45)cc3)c3cccc4c3oc3ccc(-c5ccccc5)cc34)cc2)cc1 ; c1ccc(-c2ccc3oc4c(N(c5ccc(-c6ccc7c(ccc8ccccc87)c6)cc5)c5ccc(-c6ccccc6)c6ccccc56)cccc4c3c2)cc1 ; c1ccc(-c2ccc(-c3ccc(N(c4cccc5ccccc45)c4cccc5c4oc4ccc(-c6ccccc6)cc45)cc3)cc2)cc1 ; c1ccc(-c2ccc3oc4c(N(c5cccc(-c6cccc7ccccc67)c5)c5cc6ccccc6c6ccccc56)cccc4c3c2)cc1]

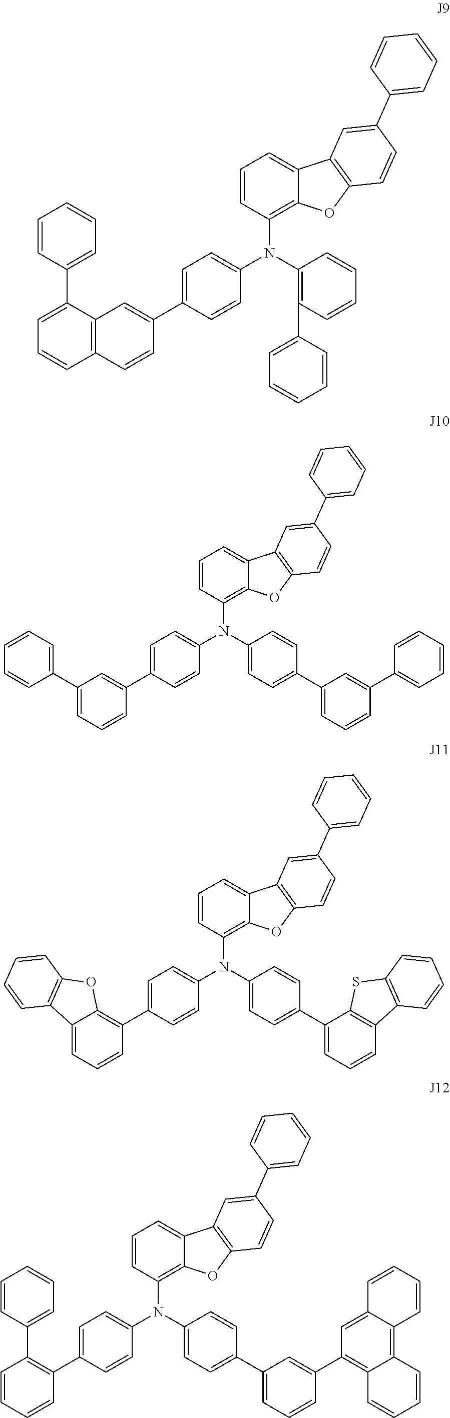

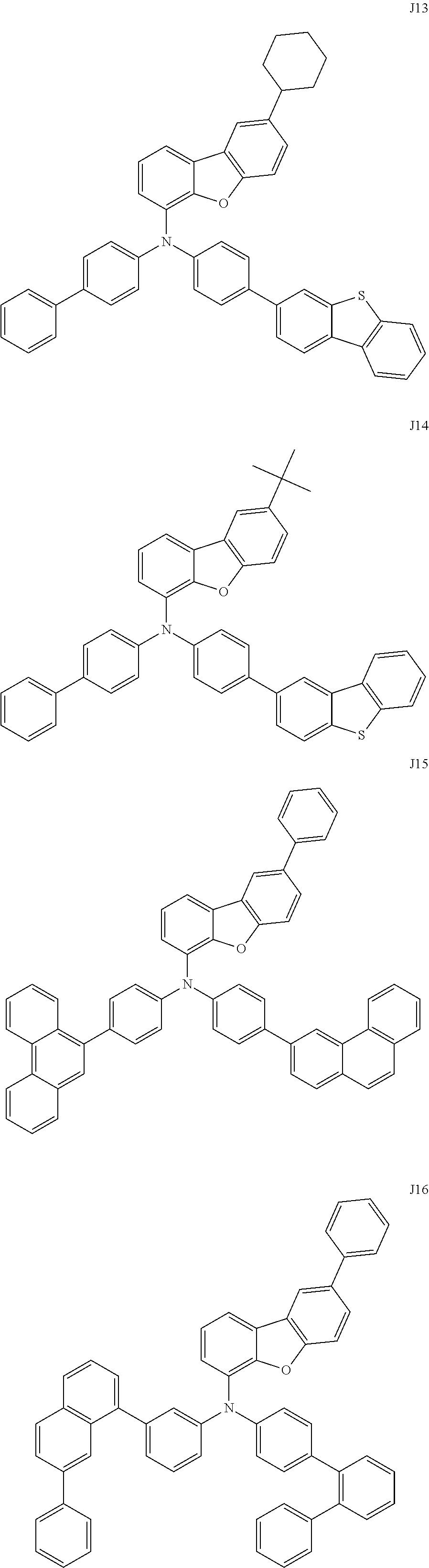 [SMILES: c1ccc(-c2ccc(N(c3ccc(-c4ccc5c(c4)sc4ccccc45)cc3)c3cccc4c3oc3ccc(C5CCCCC5)cc34)cc2)cc1 ; CC(C)(C)c1ccc2oc3c(N(c4ccc(-c5ccccc5)cc4)c4ccc(-c5ccc6sc7ccccc7c6c5)cc4)cccc3c2c1 ; c1ccc(-c2ccc3oc4c(N(c5ccc(-c6ccc7ccc8ccccc8c7c6)cc5)c5ccc(-c6cc7ccccc7c7ccccc67)cc5)cccc4c3c2)cc1 ; c1ccc(-c2ccc3cccc(-c4cccc(N(c5ccc(-c6ccccc6-c6ccccc6)cc5)c5cccc6c5oc5ccc(-c7ccccc7)cc56)c4)c3c2)cc1]

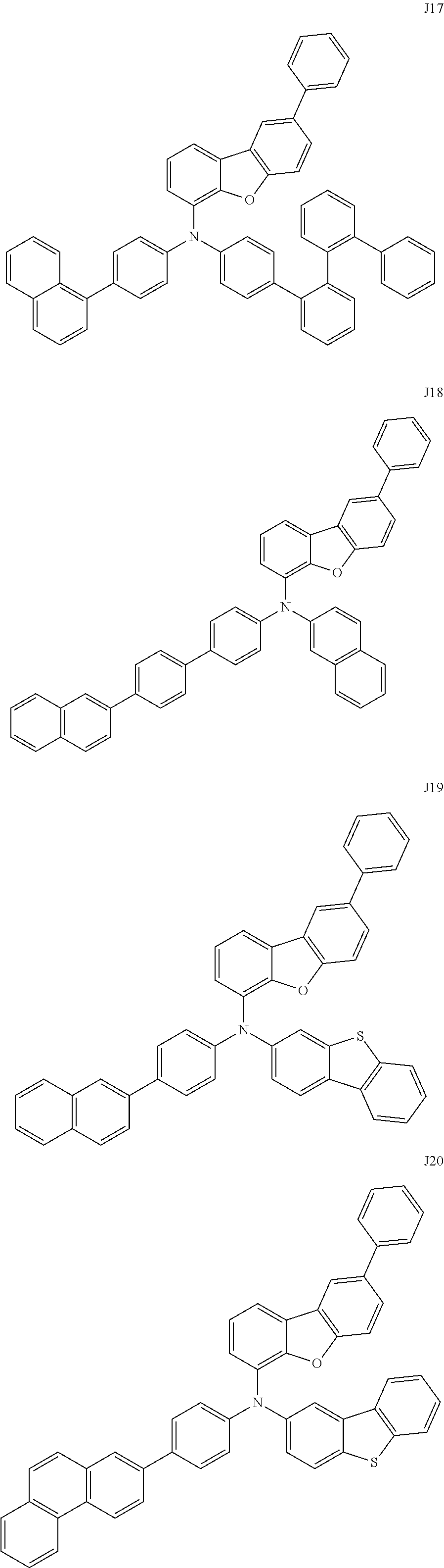 [SMILES: c1ccc(-c2ccc3oc4c(N(c5ccc(-c6ccccc6-c6ccccc6-c6ccccc6)cc5)c5ccc(-c6cccc7ccccc67)cc5)cccc4c3c2)cc1 ; c1ccc(-c2ccc3oc4c(N(c5ccc(-c6ccc(-c7ccc8ccccc8c7)cc6)cc5)c5ccc6ccccc6c5)cccc4c3c2)cc1 ; c1ccc(-c2ccc3oc4c(N(c5ccc(-c6ccc7ccccc7c6)cc5)c5ccc6c(c5)sc5ccccc56)cccc4c3c2)cc1 ; c1ccc(-c2ccc3oc4c(N(c5ccc(-c6ccc7c(ccc8ccccc87)c6)cc5)c5ccc6sc7ccccc7c6c5)cccc4c3c2)cc1]

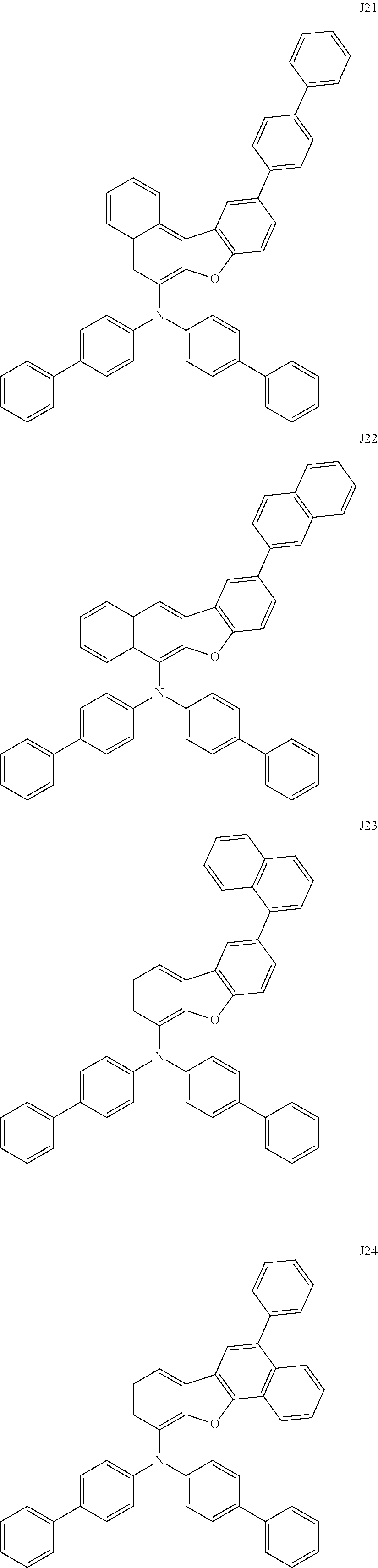 [SMILES: c1ccc(-c2ccc(-c3ccc4oc5c(N(c6ccc(-c7ccccc7)cc6)c6ccc(-c7ccccc7)cc6)cc6ccccc6c5c4c3)cc2)cc1 ; c1ccc(-c2ccc(N(c3ccc(-c4ccccc4)cc3)c3c4ccccc4cc4c3oc3ccc(-c5ccc6ccccc6c5)cc34)cc2)cc1 ; c1ccc(-c2ccc(N(c3ccc(-c4ccccc4)cc3)c3cccc4c3oc3ccc(-c5cccc6ccccc56)cc34)cc2)cc1 ; c1ccc(-c2ccc(N(c3ccc(-c4ccccc4)cc3)c3cccc4c3oc3c5ccccc5c(-c5ccccc5)cc43)cc2)cc1]

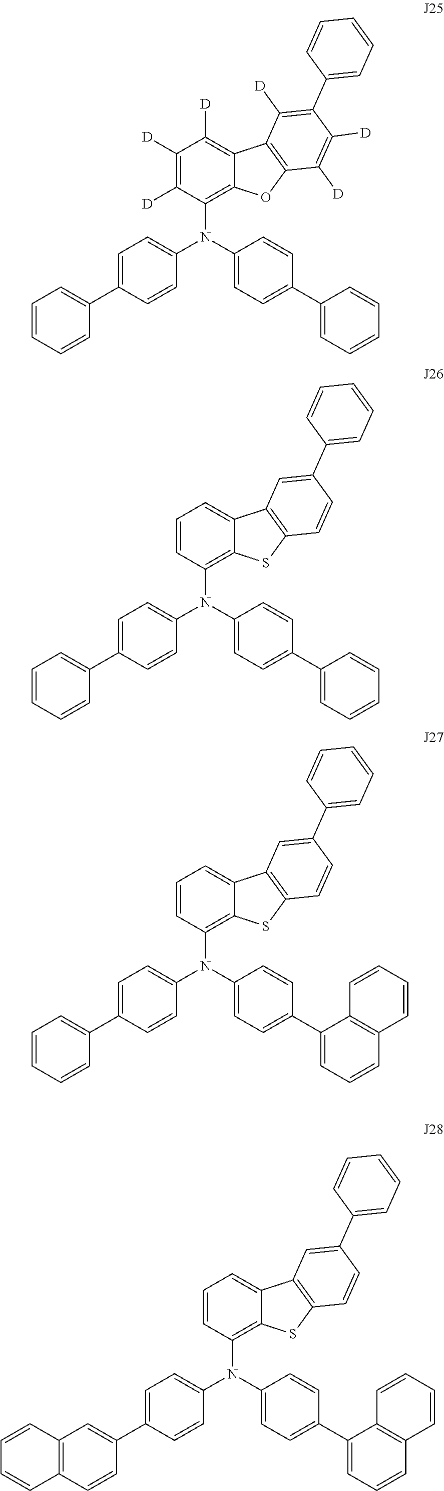

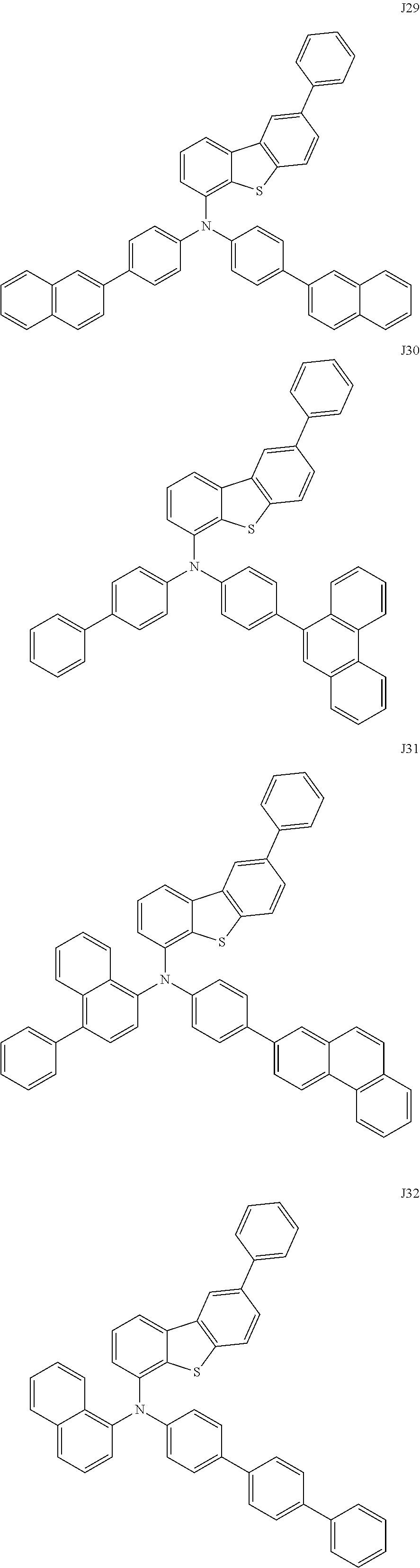

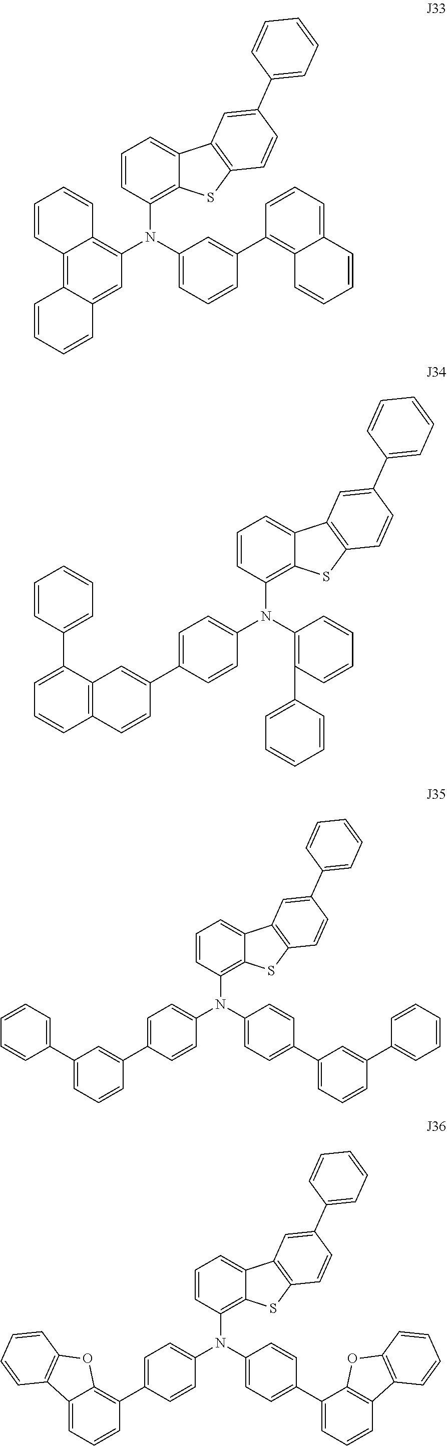 [SMILES: c1ccc(-c2ccc3sc4c(N(c5cccc(-c6cccc7ccccc67)c5)c5cc6ccccc6c6ccccc56)cccc4c3c2)cc1 ; c1ccc(-c2ccc3sc4c(N(c5ccc(-c6ccc7cccc(-c8ccccc8)c7c6)cc5)c5ccccc5-c5ccccc5)cccc4c3c2)cc1 ; c1ccc(-c2cccc(-c3ccc(N(c4ccc(-c5cccc(-c6ccccc6)c5)cc4)c4cccc5c4sc4ccc(-c6ccccc6)cc45)cc3)c2)cc1 ; c1ccc(-c2ccc3sc4c(N(c5ccc(-c6cccc7c6oc6ccccc67)cc5)c5ccc(-c6cccc7c6oc6ccccc67)cc5)cccc4c3c2)cc1]

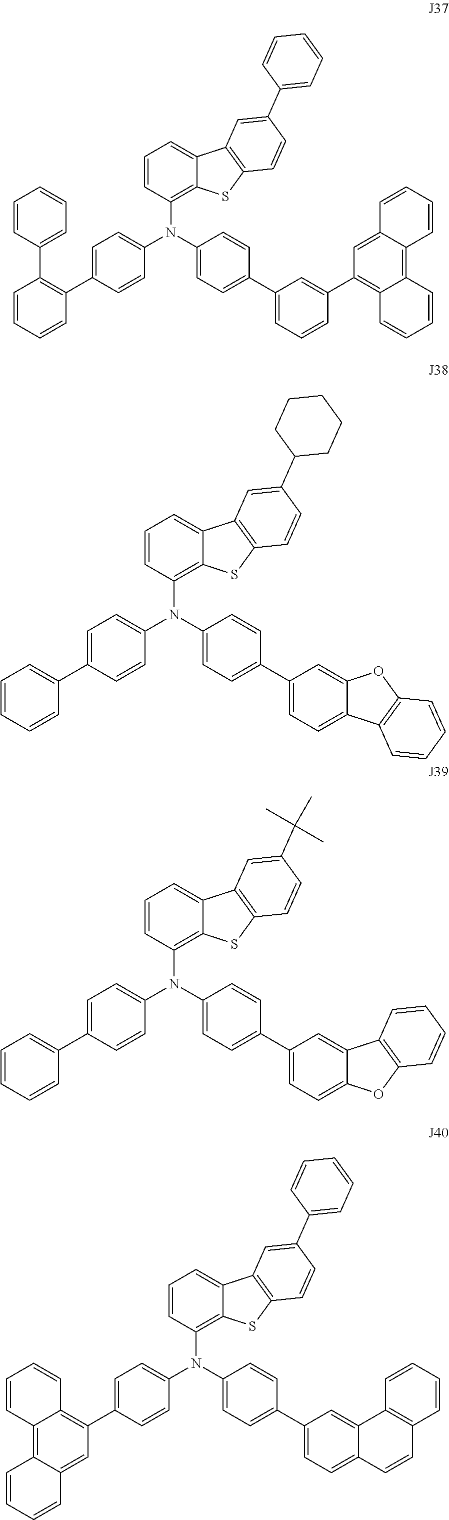 [SMILES: c1ccc(-c2ccc3sc4c(N(c5ccc(-c6cccc(-c7cc8ccccc8c8ccccc78)c6)cc5)c5ccc(-c6ccccc6-c6ccccc6)cc5)cccc4c3c2)cc1 ; c1ccc(-c2ccc(N(c3ccc(-c4ccc5c(c4)oc4ccccc45)cc3)c3cccc4c3sc3ccc(C5CCCCC5)cc34)cc2)cc1 ; CC(C)(C)c1ccc2sc3c(N(c4ccc(-c5ccccc5)cc4)c4ccc(-c5ccc6oc7ccccc7c6c5)cc4)cccc3c2c1 ; c1ccc(-c2ccc3sc4c(N(c5ccc(-c6ccc7ccc8ccccc8c7c6)cc5)c5ccc(-c6cc7ccccc7c7ccccc67)cc5)cccc4c3c2)cc1]

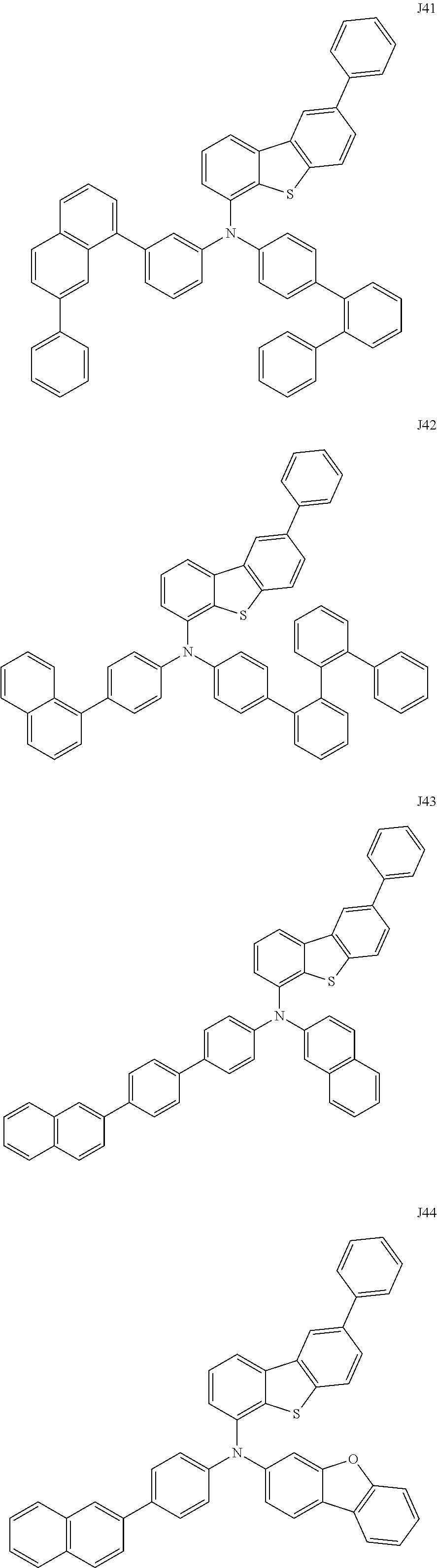 [SMILES: c1ccc(-c2ccc3cccc(-c4cccc(N(c5ccc(-c6ccccc6-c6ccccc6)cc5)c5cccc6c5sc5ccc(-c7ccccc7)cc56)c4)c3c2)cc1 ; c1ccc(-c2ccc3sc4c(N(c5ccc(-c6ccccc6-c6ccccc6-c6ccccc6)cc5)c5ccc(-c6cccc7ccccc67)cc5)cccc4c3c2)cc1 ; c1ccc(-c2ccc3sc4c(N(c5ccc(-c6ccc(-c7ccc8ccccc8c7)cc6)cc5)c5ccc6ccccc6c5)cccc4c3c2)cc1 ; c1ccc(-c2ccc3sc4c(N(c5ccc(-c6ccc7ccccc7c6)cc5)c5ccc6c(c5)oc5ccccc56)cccc4c3c2)cc1]

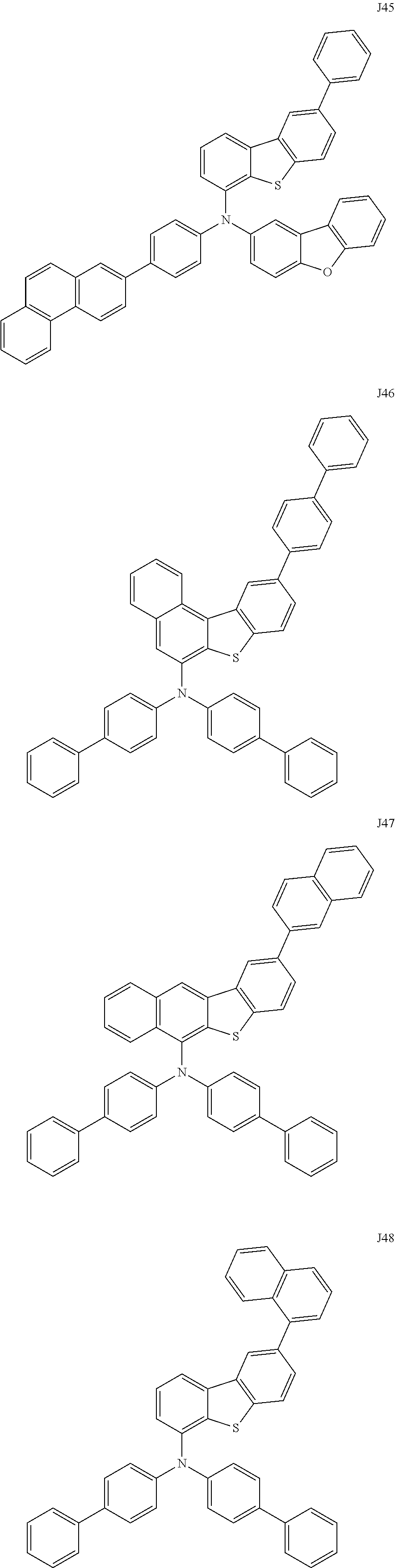 [SMILES: c1ccc(-c2ccc3sc4c(N(c5ccc(-c6ccc7c(ccc8ccccc87)c6)cc5)c5ccc6oc7ccccc7c6c5)cccc4c3c2)cc1 ; c1ccc(-c2ccc(-c3ccc4sc5c(N(c6ccc(-c7ccccc7)cc6)c6ccc(-c7ccccc7)cc6)cc6ccccc6c5c4c3)cc2)cc1 ; c1ccc(-c2ccc(N(c3ccc(-c4ccccc4)cc3)c3c4ccccc4cc4c3sc3ccc(-c5ccc6ccccc6c5)cc34)cc2)cc1 ; c1ccc(-c2ccc(N(c3ccc(-c4ccccc4)cc3)c3cccc4c3sc3ccc(-c5cccc6ccccc56)cc34)cc2)cc1]

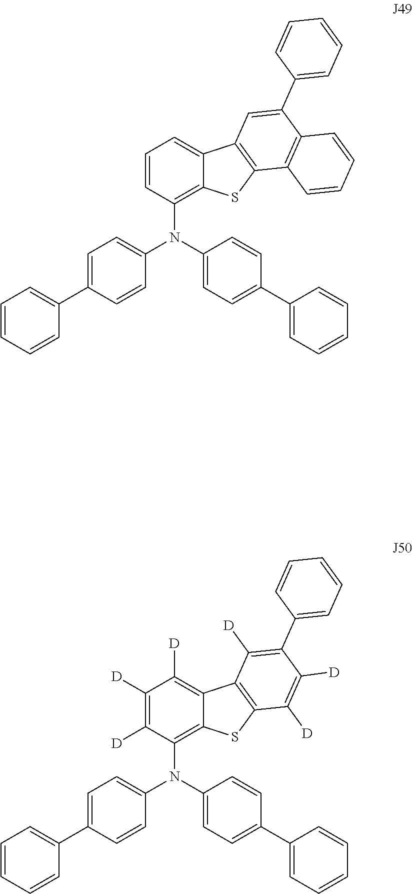 [SMILES: c1ccc(-c2ccc(N(c3ccc(-c4ccccc4)cc3)c3cccc4c3sc3c5ccccc5c(-c5ccccc5)cc43)cc2)cc1 ; [2H]c1c(-c2ccccc2)c([2H])c2c(sc3c(N(c4ccc(-c5ccccc5)cc4)c4ccc(-c5ccccc5)cc4)c([2H])c([2H])c([2H])c32)c1[2H]]

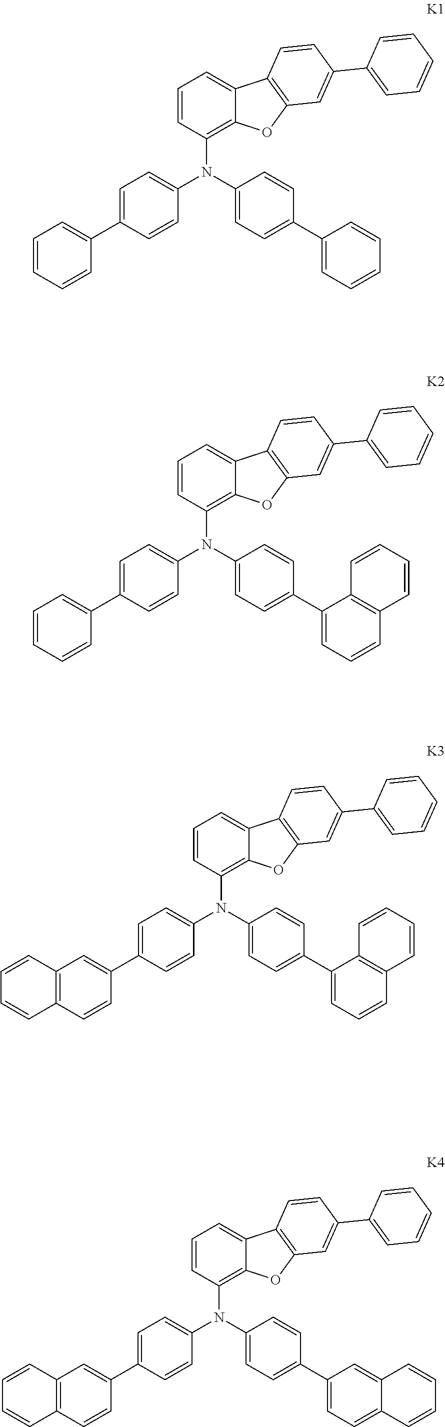 [SMILES: c1ccc(-c2ccc(N(c3ccc(-c4ccccc4)cc3)c3cccc4c3oc3cc(-c5ccccc5)ccc34)cc2)cc1 ; c1ccc(-c2ccc(N(c3ccc(-c4cccc5ccccc45)cc3)c3cccc4c3oc3cc(-c5ccccc5)ccc34)cc2)cc1 ; c1ccc(-c2ccc3c(c2)oc2c(N(c4ccc(-c5ccc6ccccc6c5)cc4)c4ccc(-c5cccc6ccccc56)cc4)cccc23)cc1 ; c1ccc(-c2ccc3c(c2)oc2c(N(c4ccc(-c5ccc6ccccc6c5)cc4)c4ccc(-c5ccc6ccccc6c5)cc4)cccc23)cc1]

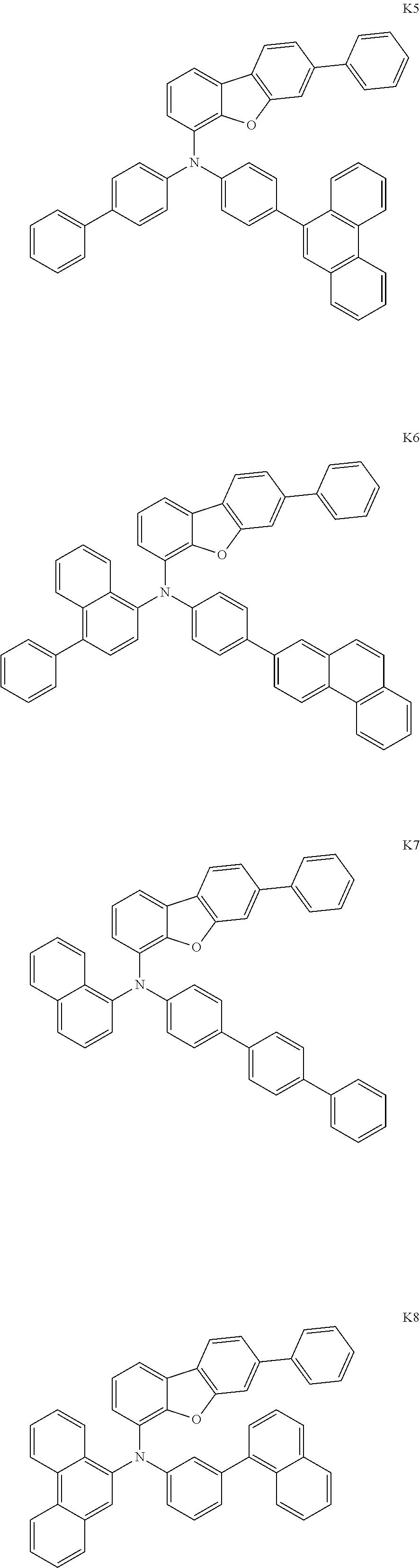 [SMILES: c1ccc(-c2ccc(N(c3ccc(-c4cc5ccccc5c5ccccc45)cc3)c3cccc4c3oc3cc(-c5ccccc5)ccc34)cc2)cc1 ; c1ccc(-c2ccc3c(c2)oc2c(N(c4ccc(-c5ccc6c(ccc7ccccc76)c5)cc4)c4ccc(-c5ccccc5)c5ccccc45)cccc23)cc1 ; c1ccc(-c2ccc(-c3ccc(N(c4cccc5ccccc45)c4cccc5c4oc4cc(-c6ccccc6)ccc45)cc3)cc2)cc1 ; c1ccc(-c2ccc3c(c2)oc2c(N(c4cccc(-c5cccc6ccccc56)c4)c4cc5ccccc5c5ccccc45)cccc23)cc1]

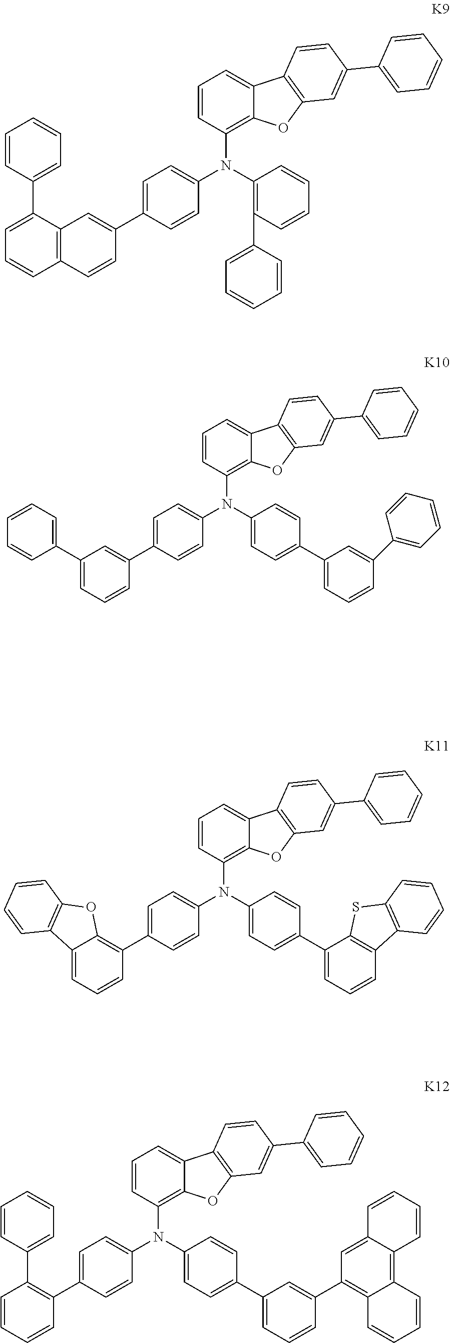

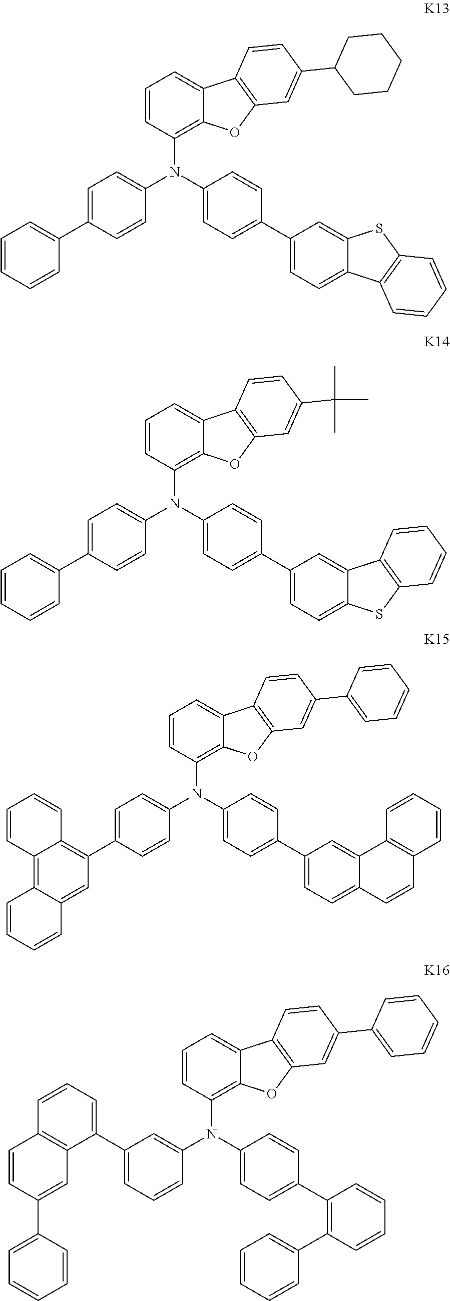 [SMILES: c1ccc(-c2ccc(N(c3ccc(-c4ccc5c(c4)sc4ccccc45)cc3)c3cccc4c3oc3cc(C5CCCCC5)ccc34)cc2)cc1 ; CC(C)(C)c1ccc2c(c1)oc1c(N(c3ccc(-c4ccccc4)cc3)c3ccc(-c4ccc5sc6ccccc6c5c4)cc3)cccc12 ; c1ccc(-c2ccc3c(c2)oc2c(N(c4ccc(-c5ccc6ccc7ccccc7c6c5)cc4)c4ccc(-c5cc6ccccc6c6ccccc56)cc4)cccc23)cc1 ; c1ccc(-c2ccc3c(c2)oc2c(N(c4ccc(-c5ccccc5-c5ccccc5)cc4)c4cccc(-c5cccc6ccc(-c7ccccc7)cc56)c4)cccc23)cc1]

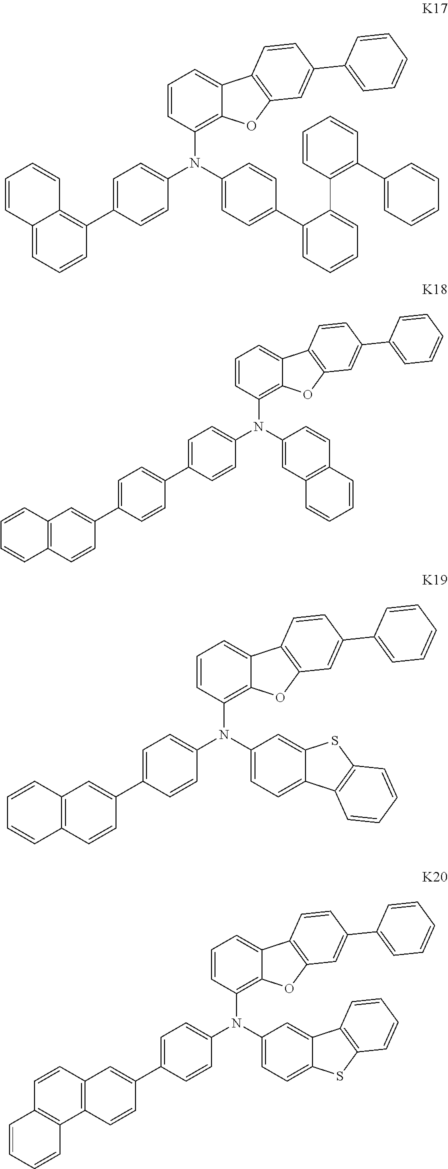 [SMILES: c1ccc(-c2ccc3c(c2)oc2c(N(c4ccc(-c5ccccc5-c5ccccc5-c5ccccc5)cc4)c4ccc(-c5cccc6ccccc56)cc4)cccc23)cc1 ; c1ccc(-c2ccc3c(c2)oc2c(N(c4ccc(-c5ccc(-c6ccc7ccccc7c6)cc5)cc4)c4ccc5ccccc5c4)cccc23)cc1 ; c1ccc(-c2ccc3c(c2)oc2c(N(c4ccc(-c5ccc6ccccc6c5)cc4)c4ccc5c(c4)sc4ccccc45)cccc23)cc1 ; c1ccc(-c2ccc3c(c2)oc2c(N(c4ccc(-c5ccc6c(ccc7ccccc76)c5)cc4)c4ccc5sc6ccccc6c5c4)cccc23)cc1]

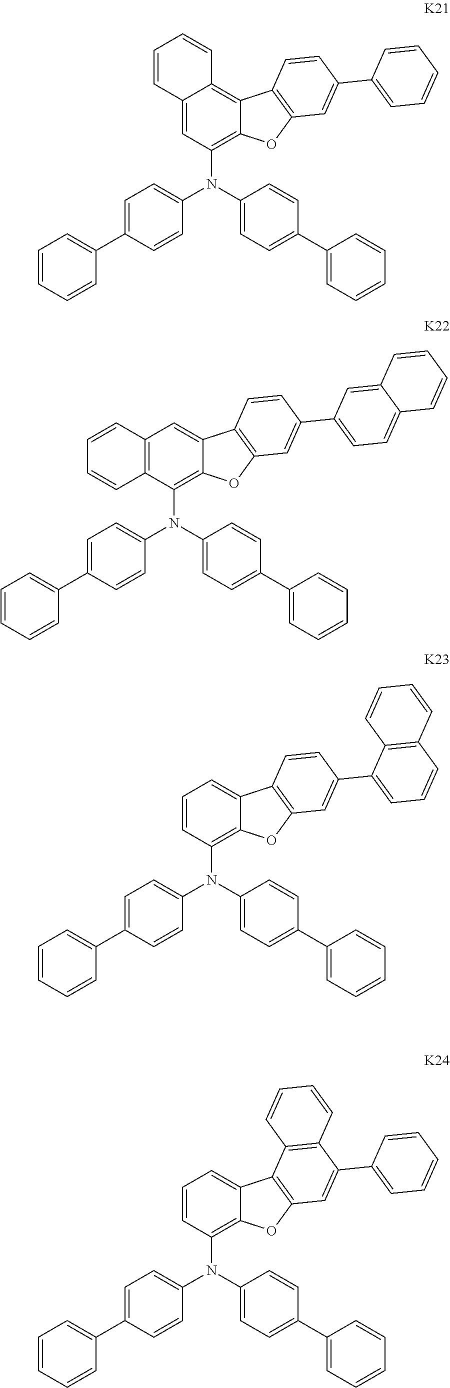 [SMILES: c1ccc(-c2ccc(N(c3ccc(-c4ccccc4)cc3)c3cc4ccccc4c4c3oc3cc(-c5ccccc5)ccc34)cc2)cc1 ; c1ccc(-c2ccc(N(c3ccc(-c4ccccc4)cc3)c3c4ccccc4cc4c3oc3cc(-c5ccc6ccccc6c5)ccc34)cc2)cc1 ; c1ccc(-c2ccc(N(c3ccc(-c4ccccc4)cc3)c3cccc4c3oc3cc(-c5cccc6ccccc56)ccc34)cc2)cc1 ; c1ccc(-c2ccc(N(c3ccc(-c4ccccc4)cc3)c3cccc4c3oc3cc(-c5ccccc5)c5ccccc5c34)cc2)cc1]

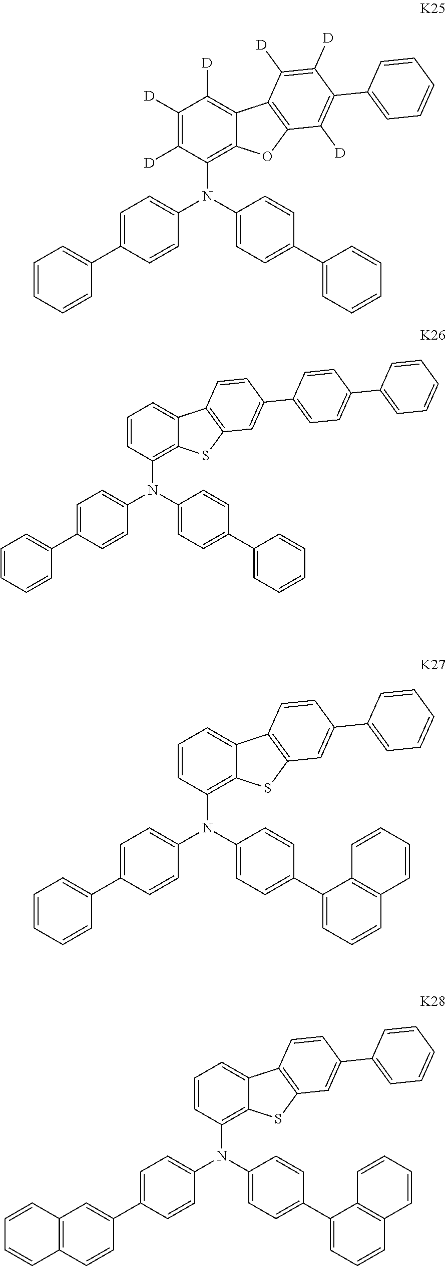 [SMILES: [2H]c1c([2H])c([2H])c2c(oc3c([2H])c(-c4ccccc4)c([2H])c([2H])c32)c1N(c1ccc(-c2ccccc2)cc1)c1ccc(-c2ccccc2)cc1 ; c1ccc(-c2ccc(-c3ccc4c(c3)sc3c(N(c5ccc(-c6ccccc6)cc5)c5ccc(-c6ccccc6)cc5)cccc34)cc2)cc1 ; c1ccc(-c2ccc(N(c3ccc(-c4cccc5ccccc45)cc3)c3cccc4c3sc3cc(-c5ccccc5)ccc34)cc2)cc1 ; c1ccc(-c2ccc3c(c2)sc2c(N(c4ccc(-c5ccc6ccccc6c5)cc4)c4ccc(-c5cccc6ccccc56)cc4)cccc23)cc1]

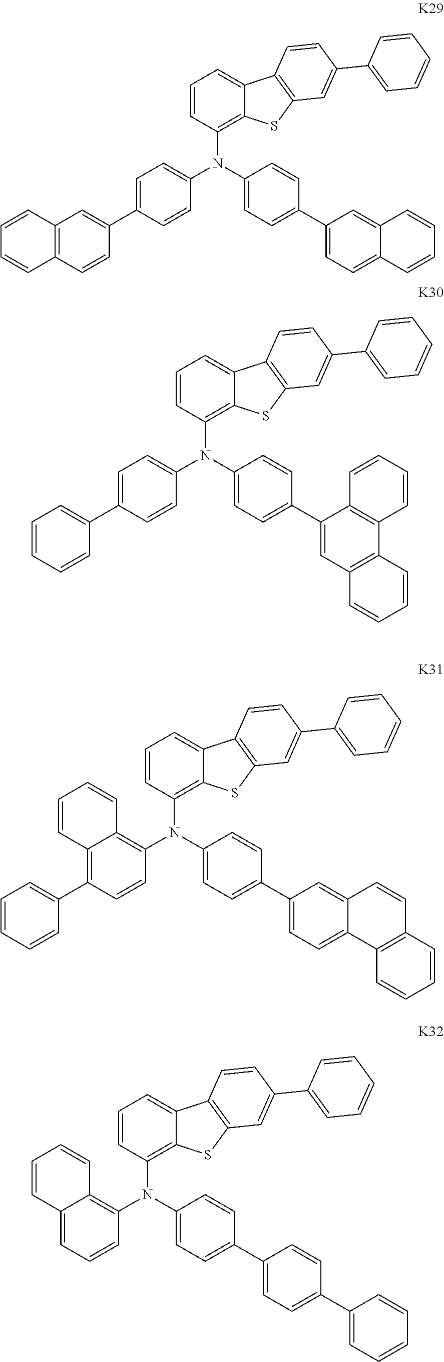

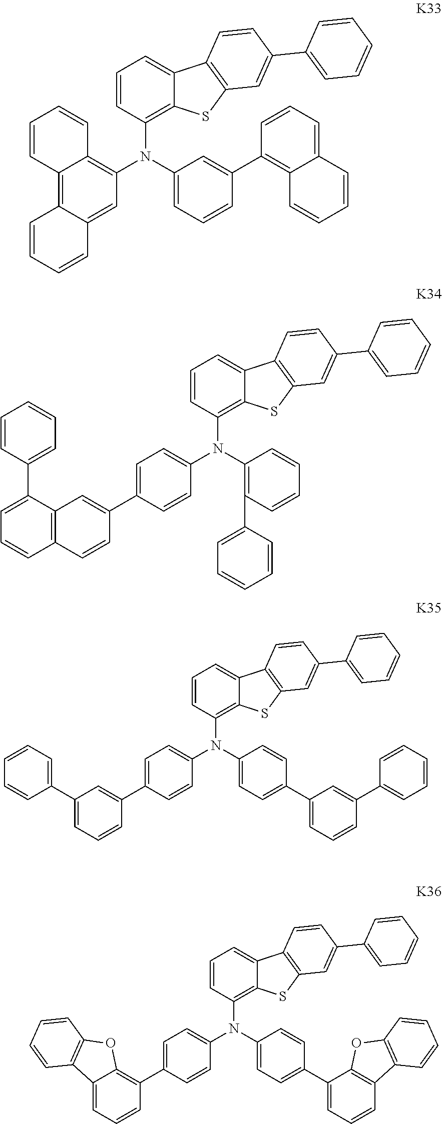 [SMILES: c1ccc(-c2ccc3c(c2)sc2c(N(c4cccc(-c5cccc6ccccc56)c4)c4cc5ccccc5c5ccccc45)cccc23)cc1 ; c1ccc(-c2ccc3c(c2)sc2c(N(c4ccc(-c5ccc6cccc(-c7ccccc7)c6c5)cc4)c4ccccc4-c4ccccc4)cccc23)cc1 ; c1ccc(-c2cccc(-c3ccc(N(c4ccc(-c5cccc(-c6ccccc6)c5)cc4)c4cccc5c4sc4cc(-c6ccccc6)ccc45)cc3)c2)cc1 ; c1ccc(-c2ccc3c(c2)sc2c(N(c4ccc(-c5cccc6c5oc5ccccc56)cc4)c4ccc(-c5cccc6c5oc5ccccc56)cc4)cccc23)cc1]

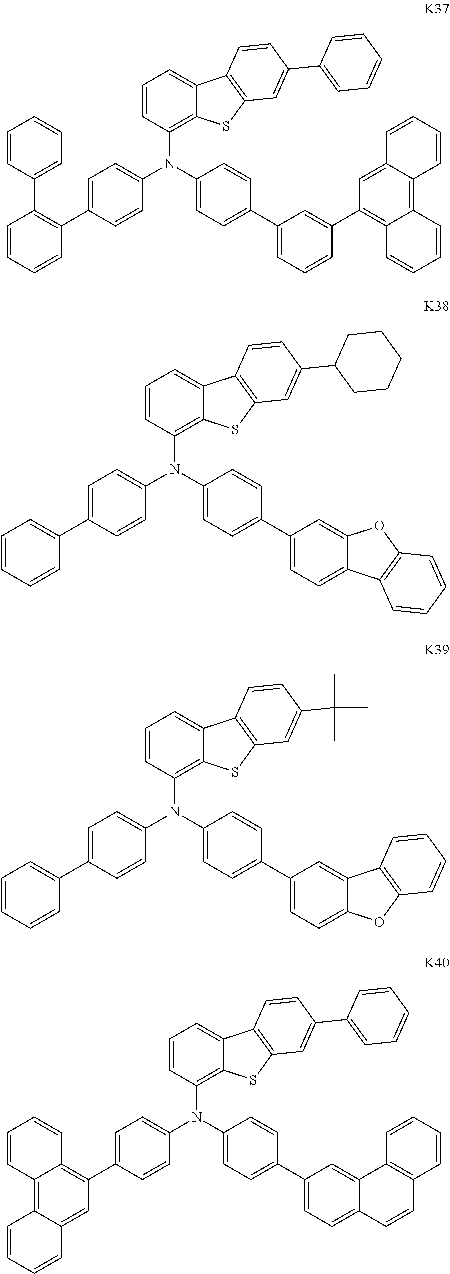 [SMILES: c1ccc(-c2ccc3c(c2)sc2c(N(c4ccc(-c5cccc(-c6cc7ccccc7c7ccccc67)c5)cc4)c4ccc(-c5ccccc5-c5ccccc5)cc4)cccc23)cc1 ; c1ccc(-c2ccc(N(c3ccc(-c4ccc5c(c4)oc4ccccc45)cc3)c3cccc4c3sc3cc(C5CCCCC5)ccc34)cc2)cc1 ; CC(C)(C)c1ccc2c(c1)sc1c(N(c3ccc(-c4ccccc4)cc3)c3ccc(-c4ccc5oc6ccccc6c5c4)cc3)cccc12 ; c1ccc(-c2ccc3c(c2)sc2c(N(c4ccc(-c5ccc6ccc7ccccc7c6c5)cc4)c4ccc(-c5cc6ccccc6c6ccccc56)cc4)cccc23)cc1]

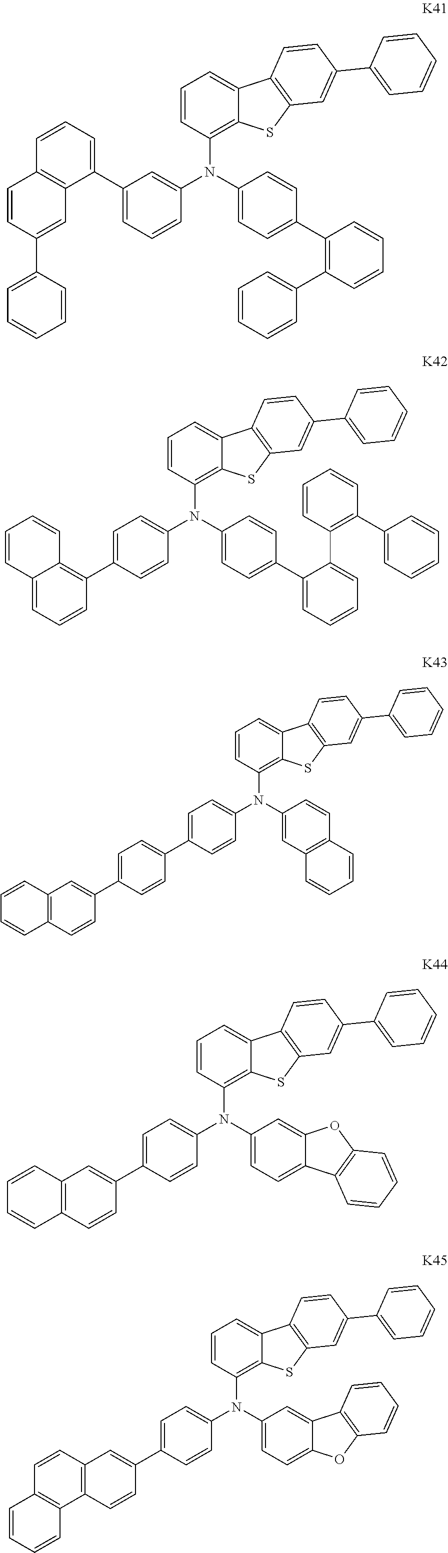 [SMILES: c1ccc(-c2ccc3c(c2)sc2c(N(c4ccc(-c5ccccc5-c5ccccc5)cc4)c4cccc(-c5cccc6ccc(-c7ccccc7)cc56)c4)cccc23)cc1 ; c1ccc(-c2ccc3c(c2)sc2c(N(c4ccc(-c5ccccc5-c5ccccc5-c5ccccc5)cc4)c4ccc(-c5cccc6ccccc56)cc4)cccc23)cc1 ; c1ccc(-c2ccc3c(c2)sc2c(N(c4ccc(-c5ccc(-c6ccc7ccccc7c6)cc5)cc4)c4ccc5ccccc5c4)cccc23)cc1 ; c1ccc(-c2ccc3c(c2)sc2c(N(c4ccc(-c5ccc6ccccc6c5)cc4)c4ccc5c(c4)oc4ccccc45)cccc23)cc1 ; c1ccc(-c2ccc3c(c2)sc2c(N(c4ccc(-c5ccc6c(ccc7ccccc76)c5)cc4)c4ccc5oc6ccccc6c5c4)cccc23)cc1]

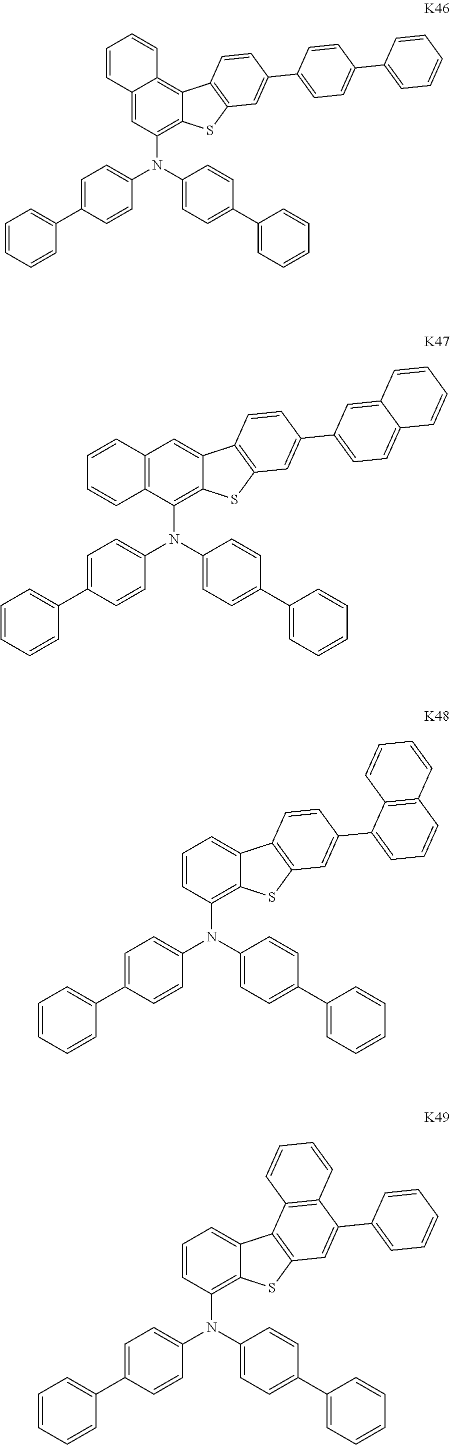 [SMILES: c1ccc(-c2ccc(-c3ccc4c(c3)sc3c(N(c5ccc(-c6ccccc6)cc5)c5ccc(-c6ccccc6)cc5)cc5ccccc5c34)cc2)cc1 ; c1ccc(-c2ccc(N(c3ccc(-c4ccccc4)cc3)c3c4ccccc4cc4c3sc3cc(-c5ccc6ccccc6c5)ccc34)cc2)cc1 ; c1ccc(-c2ccc(N(c3ccc(-c4ccccc4)cc3)c3cccc4c3sc3cc(-c5cccc6ccccc56)ccc34)cc2)cc1 ; c1ccc(-c2ccc(N(c3ccc(-c4ccccc4)cc3)c3cccc4c3sc3cc(-c5ccccc5)c5ccccc5c34)cc2)cc1]

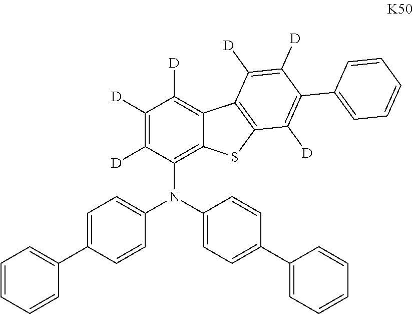 [SMILES: [2H]c1c([2H])c([2H])c2c(sc3c([2H])c(-c4ccccc4)c([2H])c([2H])c32)c1N(c1ccc(-c2ccccc2)cc1)c1ccc(-c2ccccc2)cc1]

21. The amine compound of claim 15, wherein the amine compound represented by Formula 1 is any one selected from among compounds represented in Compound Group 3: 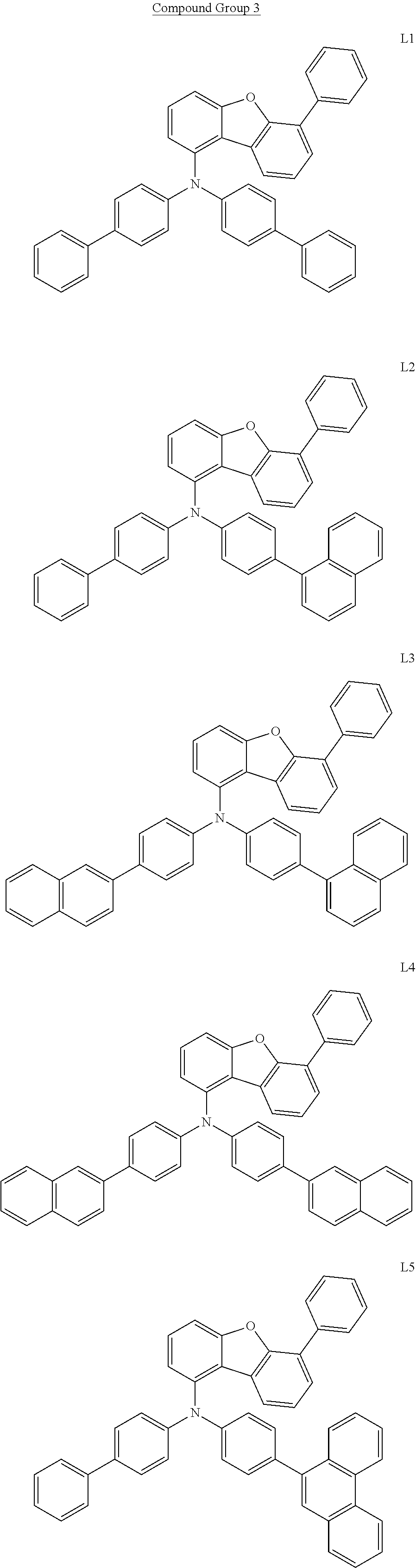 [SMILES: c1ccc(-c2ccc(N(c3ccc(-c4ccccc4)cc3)c3cccc4oc5c(-c6ccccc6)cccc5c34)cc2)cc1 ; c1ccc(-c2ccc(N(c3ccc(-c4cccc5ccccc45)cc3)c3cccc4oc5c(-c6ccccc6)cccc5c34)cc2)cc1 ; c1ccc(-c2cccc3c2oc2cccc(N(c4ccc(-c5ccc6ccccc6c5)cc4)c4ccc(-c5cccc6ccccc56)cc4)c23)cc1 ; c1ccc(-c2cccc3c2oc2cccc(N(c4ccc(-c5ccc6ccccc6c5)cc4)c4ccc(-c5ccc6ccccc6c5)cc4)c23)cc1 ; c1ccc(-c2ccc(N(c3ccc(-c4cc5ccccc5c5ccccc45)cc3)c3cccc4oc5c(-c6ccccc6)cccc5c34)cc2)cc1] 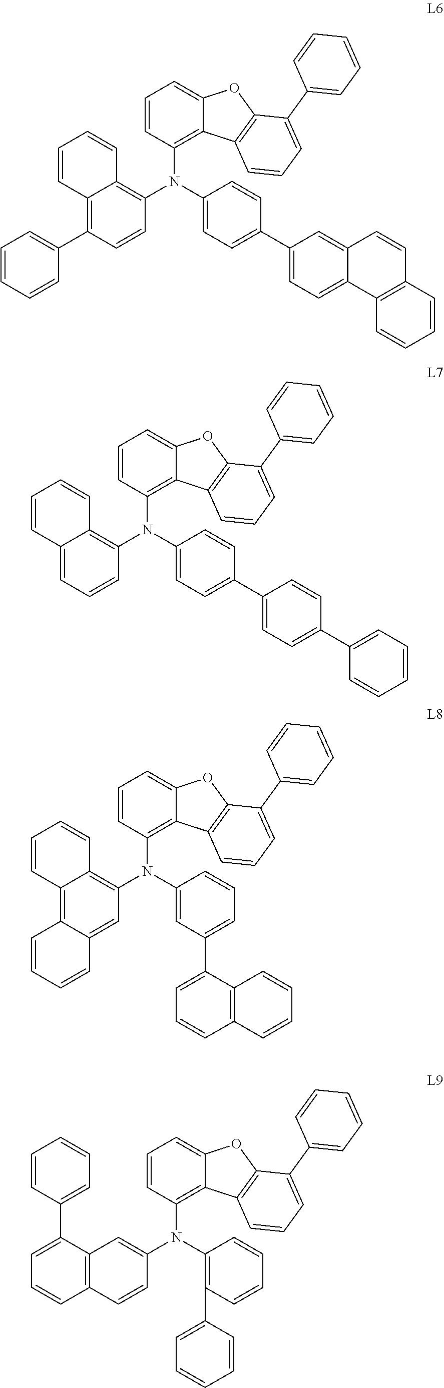 [SMILES: c1ccc(-c2ccc(N(c3ccc(-c4ccc5c(ccc6ccccc65)c4)cc3)c3cccc4oc5c(-c6ccccc6)cccc5c34)c3ccccc23)cc1 ; c1ccc(-c2ccc(-c3ccc(N(c4cccc5ccccc45)c4cccc5oc6c(-c7ccccc7)cccc6c45)cc3)cc2)cc1 ; c1ccc(-c2cccc3c2oc2cccc(N(c4cccc(-c5cccc6ccccc56)c4)c4cc5ccccc5c5ccccc45)c23)cc1 ; c1ccc(-c2ccccc2N(c2ccc3cccc(-c4ccccc4)c3c2)c2cccc3oc4c(-c5ccccc5)cccc4c23)cc1] 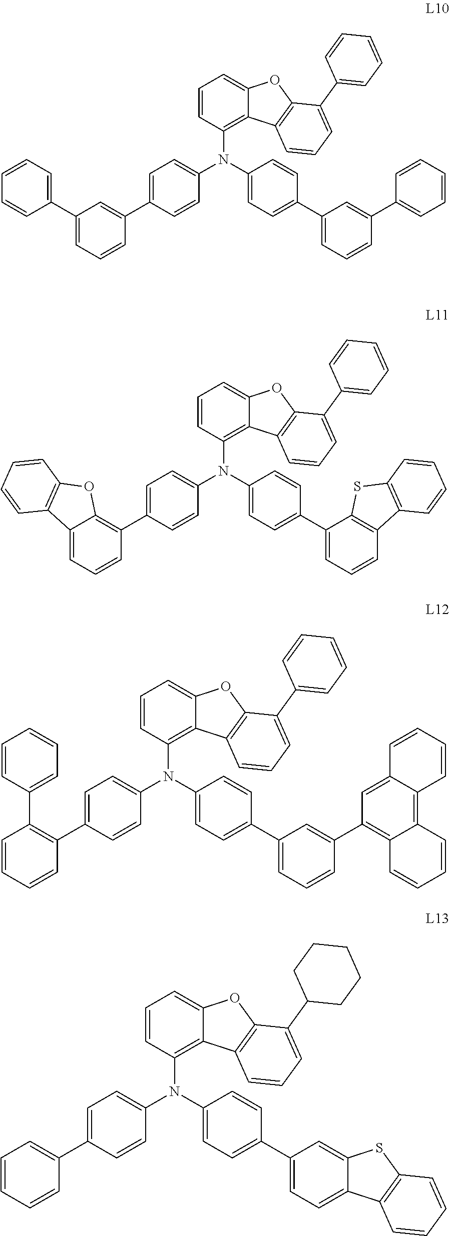 [SMILES: c1ccc(-c2cccc(-c3ccc(N(c4ccc(-c5cccc(-c6ccccc6)c5)cc4)c4cccc5oc6c(-c7ccccc7)cccc6c45)cc3)c2)cc1 ; c1ccc(-c2cccc3c2oc2cccc(N(c4ccc(-c5cccc6c5oc5ccccc56)cc4)c4ccc(-c5cccc6c5sc5ccccc56)cc4)c23)cc1 ; c1ccc(-c2ccccc2-c2ccc(N(c3ccc(-c4cccc(-c5cc6ccccc6c6ccccc56)c4)cc3)c3cccc4oc5c(-c6ccccc6)cccc5c34)cc2)cc1 ; c1ccc(-c2ccc(N(c3ccc(-c4ccc5c(c4)sc4ccccc45)cc3)c3cccc4oc5c(C6CCCCC6)cccc5c34)cc2)cc1] 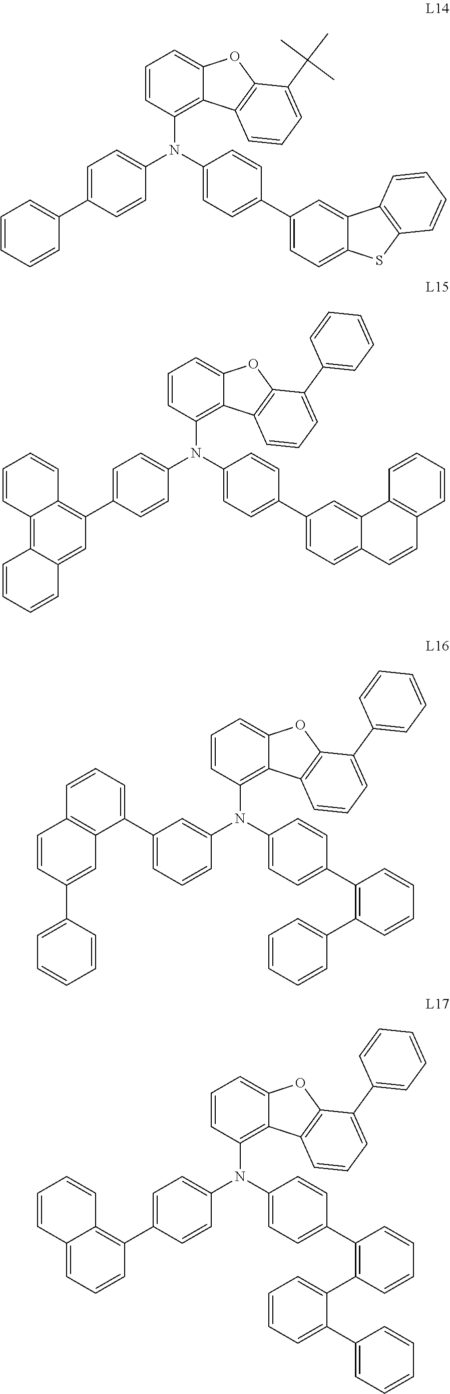 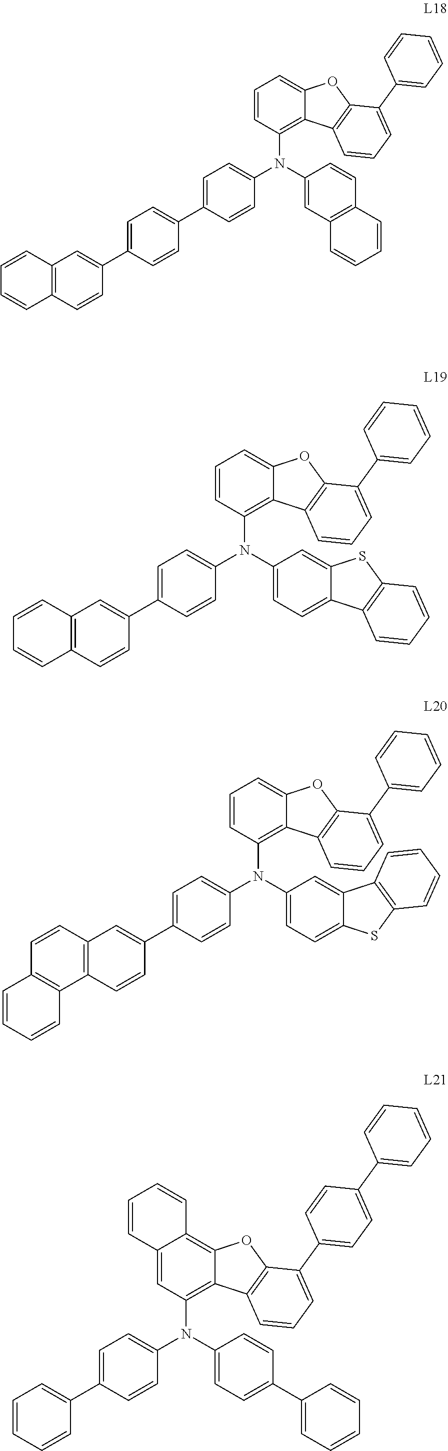 [SMILES: c1ccc(-c2cccc3c2oc2cccc(N(c4ccc(-c5ccc(-c6ccc7ccccc7c6)cc5)cc4)c4ccc5ccccc5c4)c23)cc1 ; c1ccc(-c2cccc3c2oc2cccc(N(c4ccc(-c5ccc6ccccc6c5)cc4)c4ccc5c(c4)sc4ccccc45)c23)cc1 ; c1ccc(-c2cccc3c2oc2cccc(N(c4ccc(-c5ccc6c(ccc7ccccc76)c5)cc4)c4ccc5sc6ccccc6c5c4)c23)cc1 ; c1ccc(-c2ccc(-c3cccc4c3oc3c5ccccc5cc(N(c5ccc(-c6ccccc6)cc5)c5ccc(-c6ccccc6)cc5)c43)cc2)cc1] 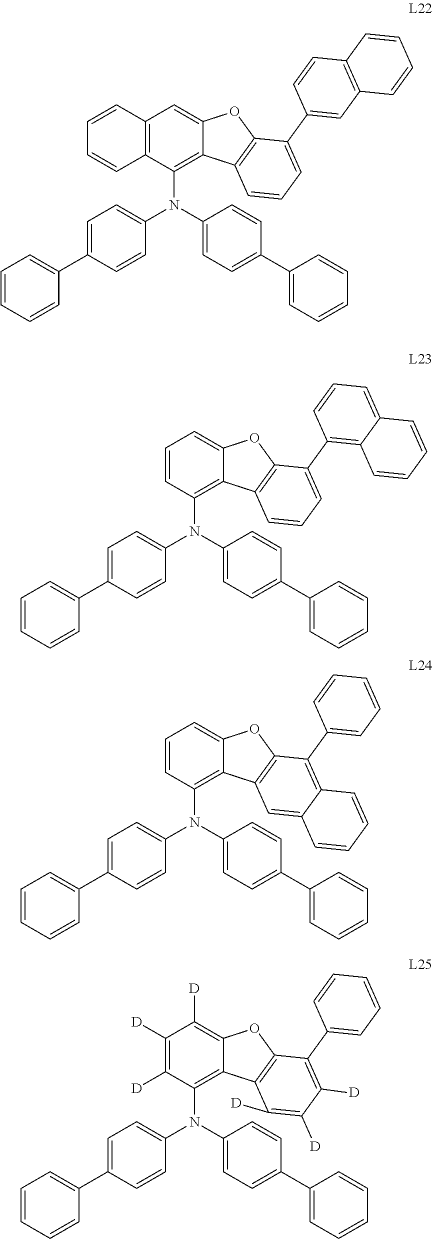 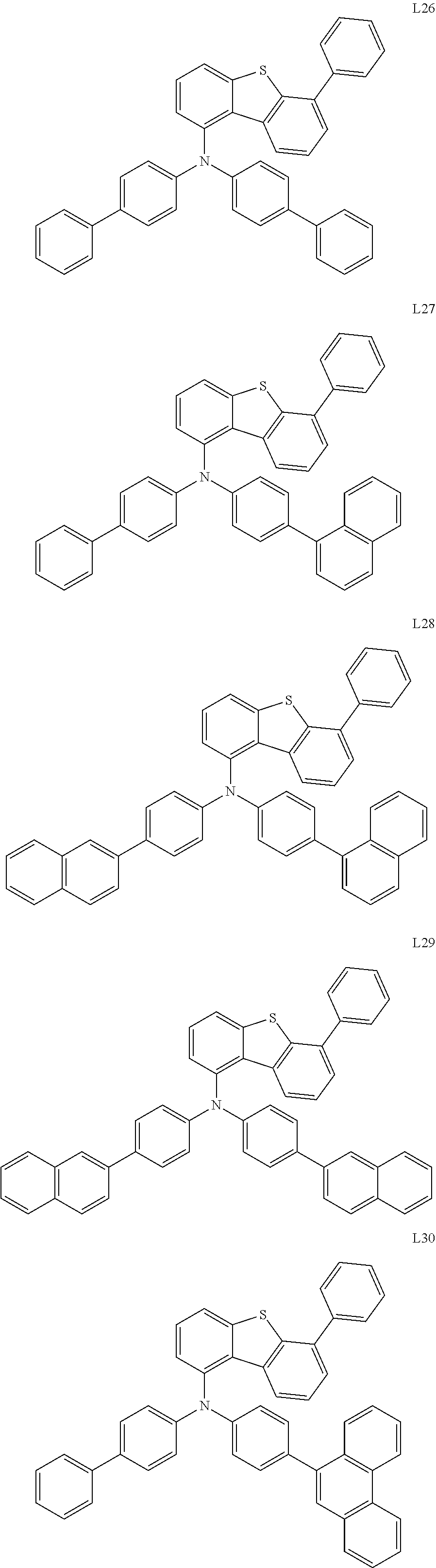 [SMILES: c1ccc(-c2ccc(N(c3ccc(-c4ccccc4)cc3)c3cccc4sc5c(-c6ccccc6)cccc5c34)cc2)cc1 ; c1ccc(-c2ccc(N(c3ccc(-c4cccc5ccccc45)cc3)c3cccc4sc5c(-c6ccccc6)cccc5c34)cc2)cc1 ; c1ccc(-c2cccc3c2sc2cccc(N(c4ccc(-c5ccc6ccccc6c5)cc4)c4ccc(-c5cccc6ccccc56)cc4)c23)cc1 ; c1ccc(-c2cccc3c2sc2cccc(N(c4ccc(-c5ccc6ccccc6c5)cc4)c4ccc(-c5ccc6ccccc6c5)cc4)c23)cc1 ; c1ccc(-c2ccc(N(c3ccc(-c4cc5ccccc5c5ccccc45)cc3)c3cccc4sc5c(-c6ccccc6)cccc5c34)cc2)cc1] 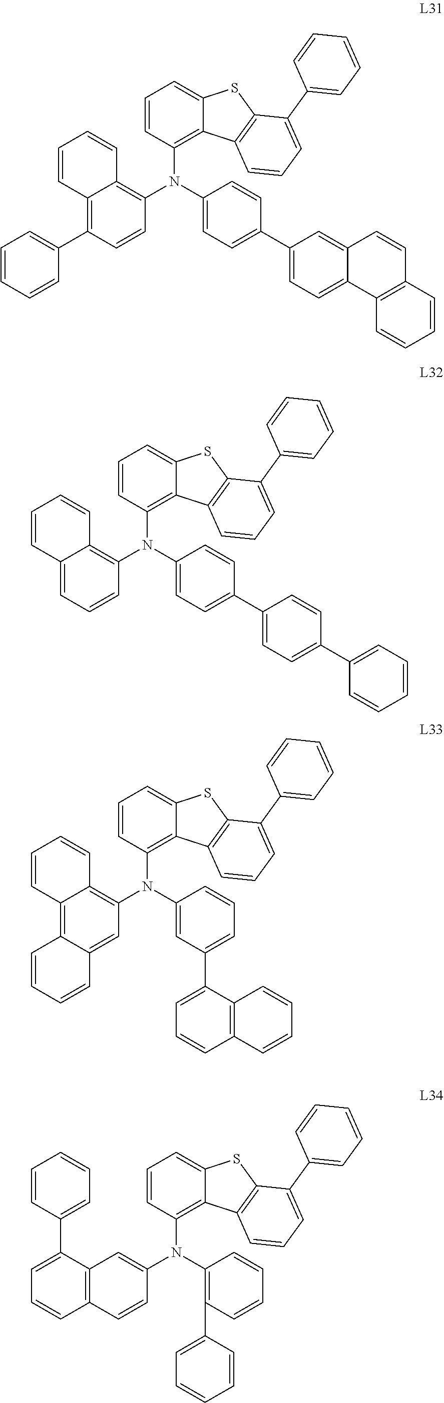 [SMILES: c1ccc(-c2ccc(N(c3ccc(-c4ccc5c(ccc6ccccc65)c4)cc3)c3cccc4sc5c(-c6ccccc6)cccc5c34)c3ccccc23)cc1 ; c1ccc(-c2ccc(-c3ccc(N(c4cccc5ccccc45)c4cccc5sc6c(-c7ccccc7)cccc6c45)cc3)cc2)cc1 ; c1ccc(-c2cccc3c2sc2cccc(N(c4cccc(-c5cccc6ccccc56)c4)c4cc5ccccc5c5ccccc45)c23)cc1 ; c1ccc(-c2ccccc2N(c2ccc3cccc(-c4ccccc4)c3c2)c2cccc3sc4c(-c5ccccc5)cccc4c23)cc1] 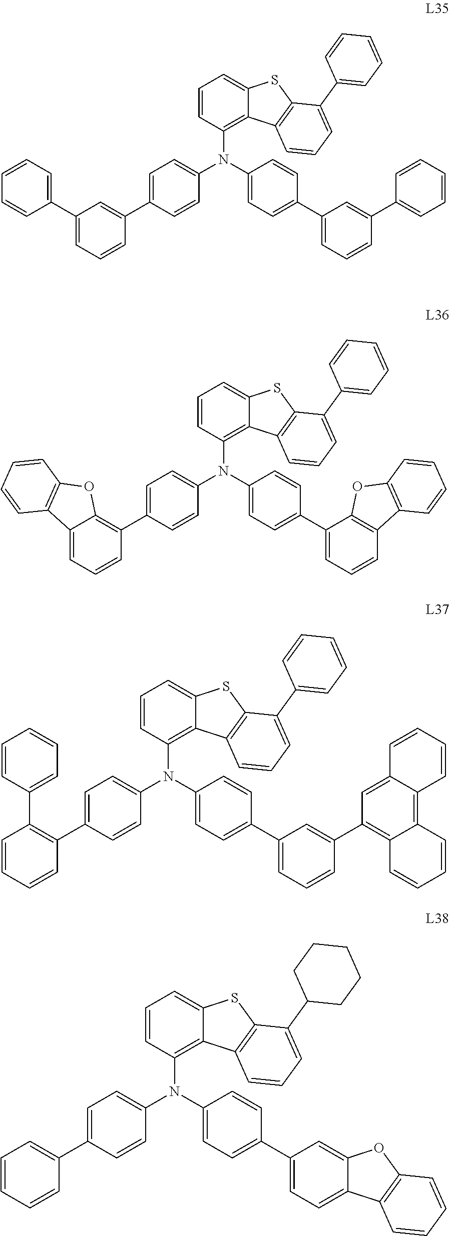 [SMILES: c1ccc(-c2cccc(-c3ccc(N(c4ccc(-c5cccc(-c6ccccc6)c5)cc4)c4cccc5sc6c(-c7ccccc7)cccc6c45)cc3)c2)cc1 ; c1ccc(-c2cccc3c2sc2cccc(N(c4ccc(-c5cccc6c5oc5ccccc56)cc4)c4ccc(-c5cccc6c5oc5ccccc56)cc4)c23)cc1 ; c1ccc(-c2ccccc2-c2ccc(N(c3ccc(-c4cccc(-c5cc6ccccc6c6ccccc56)c4)cc3)c3cccc4sc5c(-c6ccccc6)cccc5c34)cc2)cc1 ; c1ccc(-c2ccc(N(c3ccc(-c4ccc5c(c4)oc4ccccc45)cc3)c3cccc4sc5c(C6CCCCC6)cccc5c34)cc2)cc1] 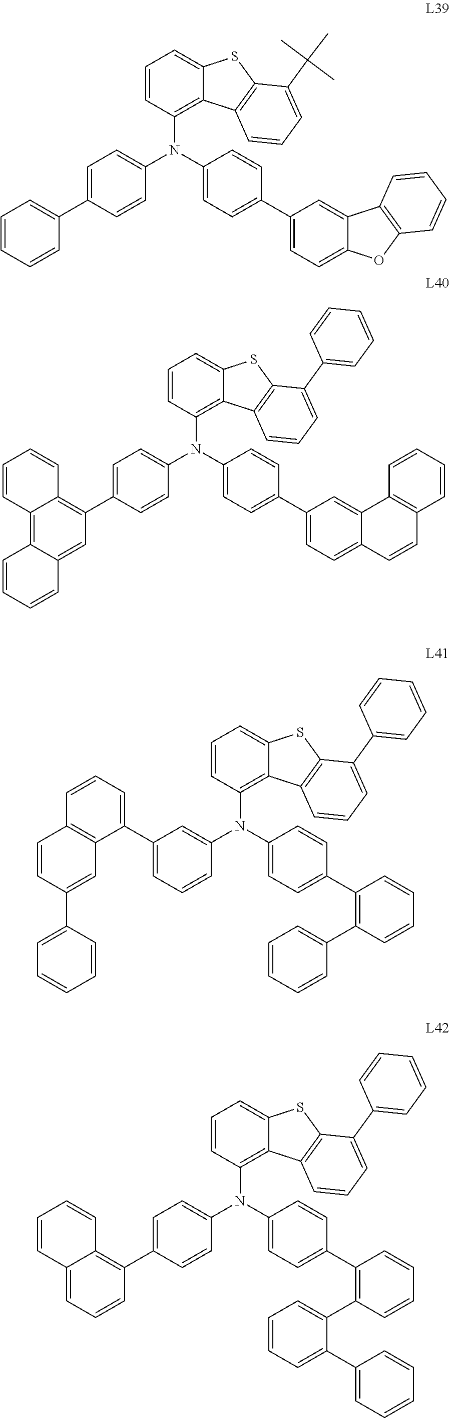 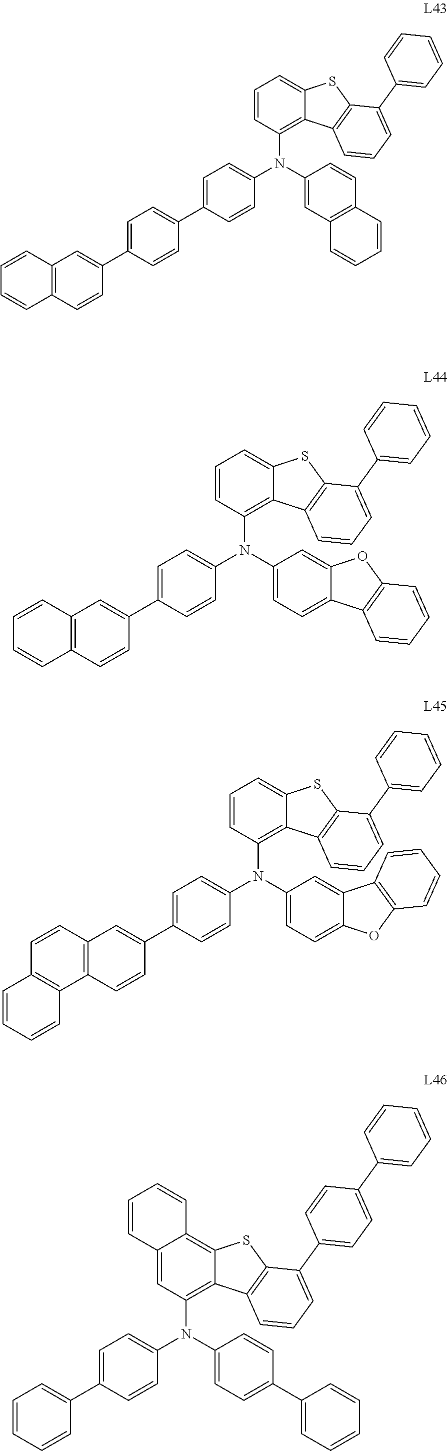 [SMILES: c1ccc(-c2cccc3c2sc2cccc(N(c4ccc(-c5ccc(-c6ccc7ccccc7c6)cc5)cc4)c4ccc5ccccc5c4)c23)cc1 ; c1ccc(-c2cccc3c2sc2cccc(N(c4ccc(-c5ccc6ccccc6c5)cc4)c4ccc5c(c4)oc4ccccc45)c23)cc1 ; c1ccc(-c2cccc3c2sc2cccc(N(c4ccc(-c5ccc6c(ccc7ccccc76)c5)cc4)c4ccc5oc6ccccc6c5c4)c23)cc1 ; c1ccc(-c2ccc(-c3cccc4c3sc3c5ccccc5cc(N(c5ccc(-c6ccccc6)cc5)c5ccc(-c6ccccc6)cc5)c43)cc2)cc1] 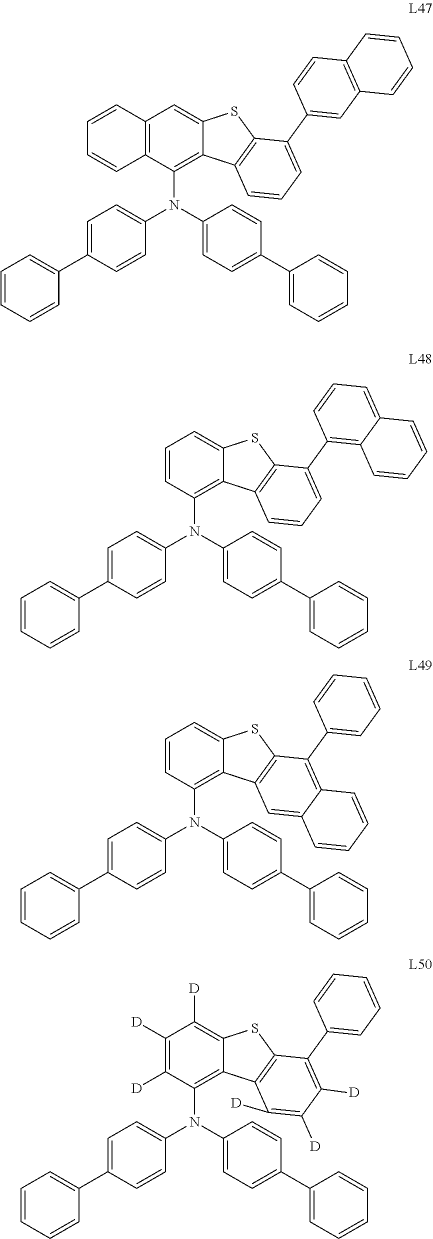 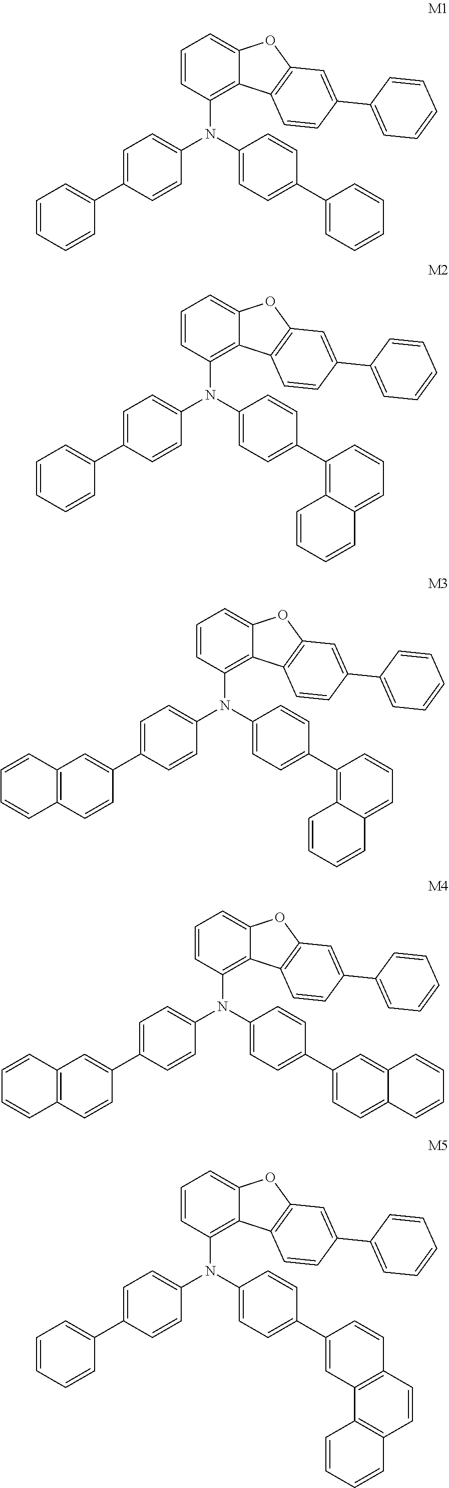 [SMILES: c1ccc(-c2ccc(N(c3ccc(-c4ccccc4)cc3)c3cccc4oc5cc(-c6ccccc6)ccc5c34)cc2)cc1 ; c1ccc(-c2ccc(N(c3ccc(-c4cccc5ccccc45)cc3)c3cccc4oc5cc(-c6ccccc6)ccc5c34)cc2)cc1 ; c1ccc(-c2ccc3c(c2)oc2cccc(N(c4ccc(-c5ccc6ccccc6c5)cc4)c4ccc(-c5cccc6ccccc56)cc4)c23)cc1 ; c1ccc(-c2ccc3c(c2)oc2cccc(N(c4ccc(-c5ccc6ccccc6c5)cc4)c4ccc(-c5ccc6ccccc6c5)cc4)c23)cc1 ; c1ccc(-c2ccc(N(c3ccc(-c4ccc5ccc6ccccc6c5c4)cc3)c3cccc4oc5cc(-c6ccccc6)ccc5c34)cc2)cc1] 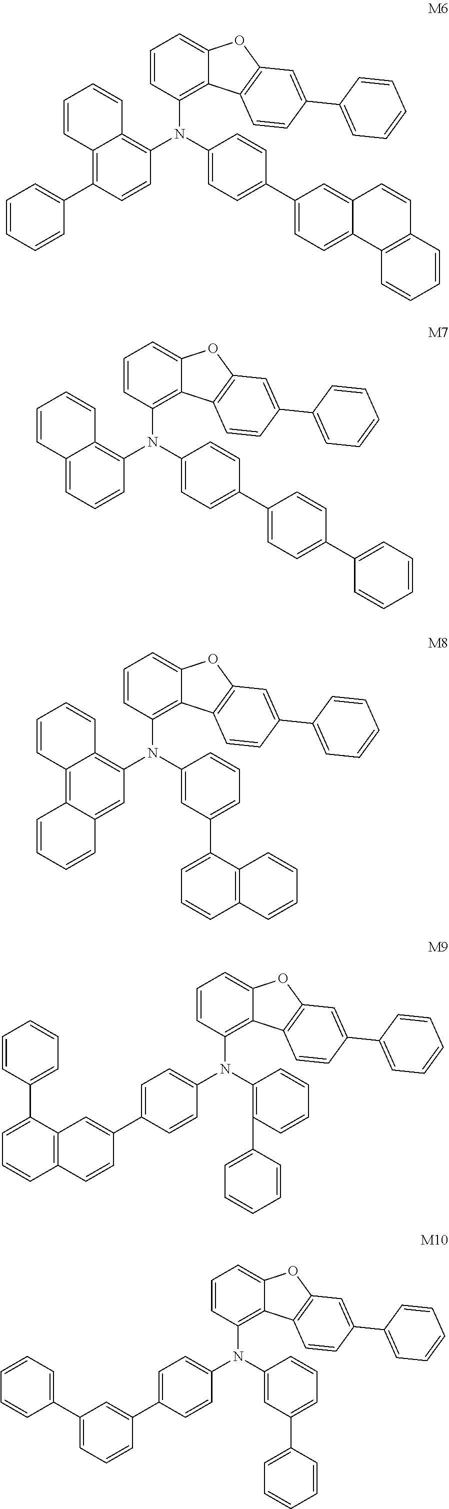 [SMILES: c1ccc(-c2ccc3c(c2)oc2cccc(N(c4ccc(-c5ccc6c(ccc7ccccc76)c5)cc4)c4ccc(-c5ccccc5)c5ccccc45)c23)cc1 ; c1ccc(-c2ccc(-c3ccc(N(c4cccc5ccccc45)c4cccc5oc6cc(-c7ccccc7)ccc6c45)cc3)cc2)cc1 ; c1ccc(-c2ccc3c(c2)oc2cccc(N(c4cccc(-c5cccc6ccccc56)c4)c4cc5ccccc5c5ccccc45)c23)cc1 ; c1ccc(-c2ccc3c(c2)oc2cccc(N(c4ccc(-c5ccc6cccc(-c7ccccc7)c6c5)cc4)c4ccccc4-c4ccccc4)c23)cc1 ; c1ccc(-c2cccc(-c3ccc(N(c4cccc(-c5ccccc5)c4)c4cccc5oc6cc(-c7ccccc7)ccc6c45)cc3)c2)cc1] 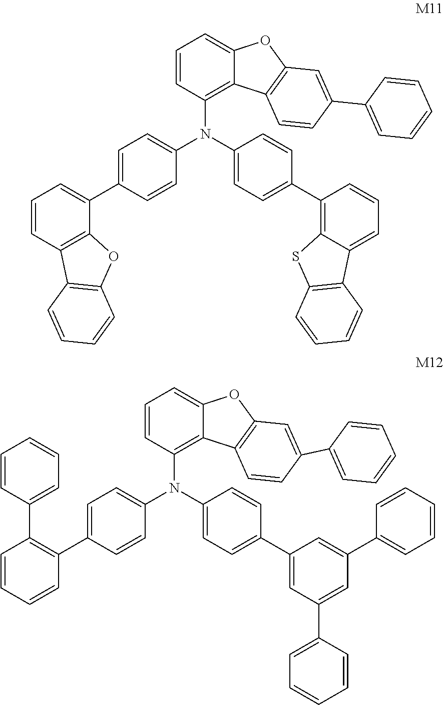 [SMILES: c1ccc(-c2ccc3c(c2)oc2cccc(N(c4ccc(-c5cccc6c5oc5ccccc56)cc4)c4ccc(-c5cccc6c5sc5ccccc56)cc4)c23)cc1 ; c1ccc(-c2cc(-c3ccccc3)cc(-c3ccc(N(c4ccc(-c5ccccc5-c5ccccc5)cc4)c4cccc5oc6cc(-c7ccccc7)ccc6c45)cc3)c2)cc1] 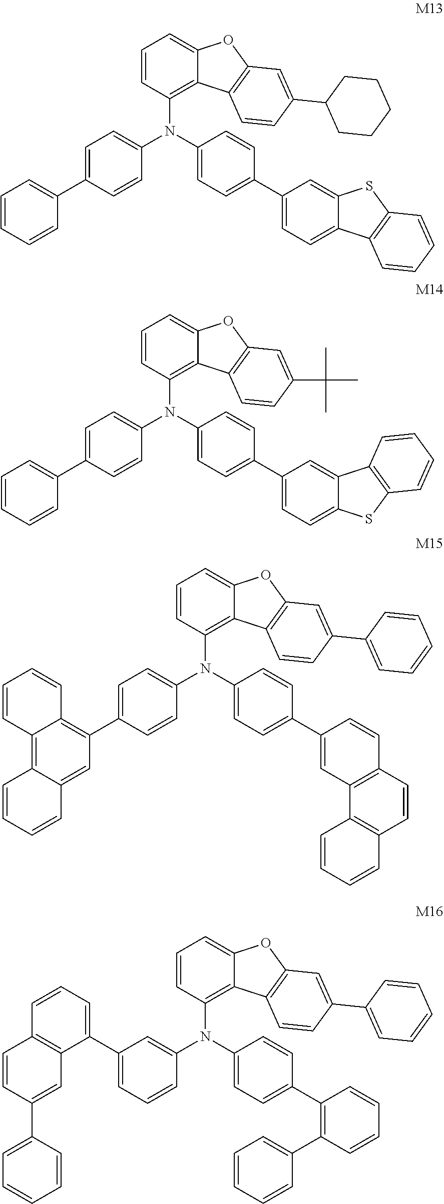 [SMILES: c1ccc(-c2ccc(N(c3ccc(-c4ccc5c(c4)sc4ccccc45)cc3)c3cccc4oc5cc(C6CCCCC6)ccc5c34)cc2)cc1 ; CC(C)(C)c1ccc2c(c1)oc1cccc(N(c3ccc(-c4ccccc4)cc3)c3ccc(-c4ccc5sc6ccccc6c5c4)cc3)c12 ; c1ccc(-c2ccc3c(c2)oc2cccc(N(c4ccc(-c5ccc6ccc7ccccc7c6c5)cc4)c4ccc(-c5cc6ccccc6c6ccccc56)cc4)c23)cc1 ; c1ccc(-c2ccc3c(c2)oc2cccc(N(c4ccc(-c5ccccc5-c5ccccc5)cc4)c4cccc(-c5cccc6ccc(-c7ccccc7)cc56)c4)c23)cc1] 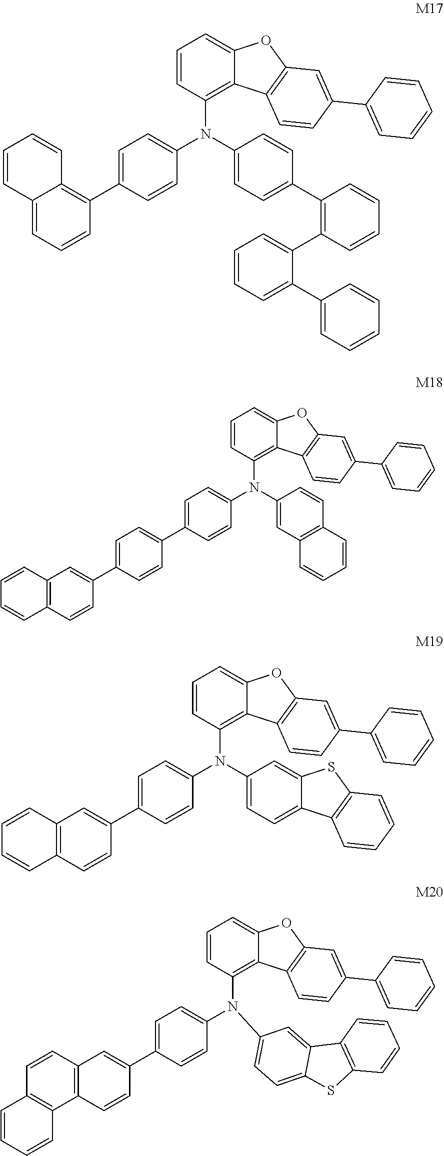 [SMILES: c1ccc(-c2ccc3c(c2)oc2cccc(N(c4ccc(-c5ccccc5-c5ccccc5-c5ccccc5)cc4)c4ccc(-c5cccc6ccccc56)cc4)c23)cc1 ; c1ccc(-c2ccc3c(c2)oc2cccc(N(c4ccc(-c5ccc(-c6ccc7ccccc7c6)cc5)cc4)c4ccc5ccccc5c4)c23)cc1 ; c1ccc(-c2ccc3c(c2)oc2cccc(N(c4ccc(-c5ccc6ccccc6c5)cc4)c4ccc5c(c4)sc4ccccc45)c23)cc1 ; c1ccc(-c2ccc3c(c2)oc2cccc(N(c4ccc(-c5ccc6c(ccc7ccccc76)c5)cc4)c4ccc5sc6ccccc6c5c4)c23)cc1] 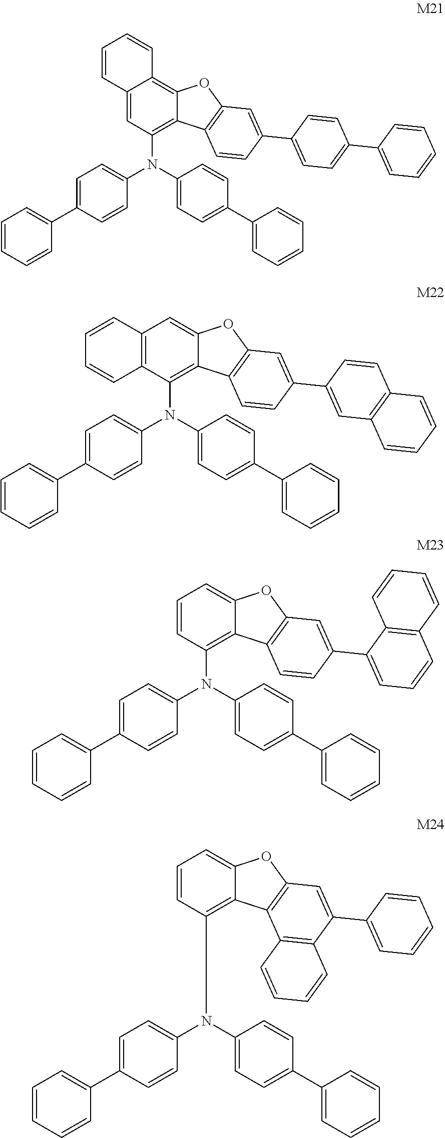 [SMILES: c1ccc(-c2ccc(-c3ccc4c(c3)oc3c5ccccc5cc(N(c5ccc(-c6ccccc6)cc5)c5ccc(-c6ccccc6)cc5)c43)cc2)cc1 ; c1ccc(-c2ccc(N(c3ccc(-c4ccccc4)cc3)c3c4ccccc4cc4oc5cc(-c6ccc7ccccc7c6)ccc5c34)cc2)cc1 ; c1ccc(-c2ccc(N(c3ccc(-c4ccccc4)cc3)c3cccc4oc5cc(-c6cccc7ccccc67)ccc5c34)cc2)cc1 ; c1ccc(-c2ccc(N(c3ccc(-c4ccccc4)cc3)c3cccc4oc5cc(-c6ccccc6)c6ccccc6c5c34)cc2)cc1] 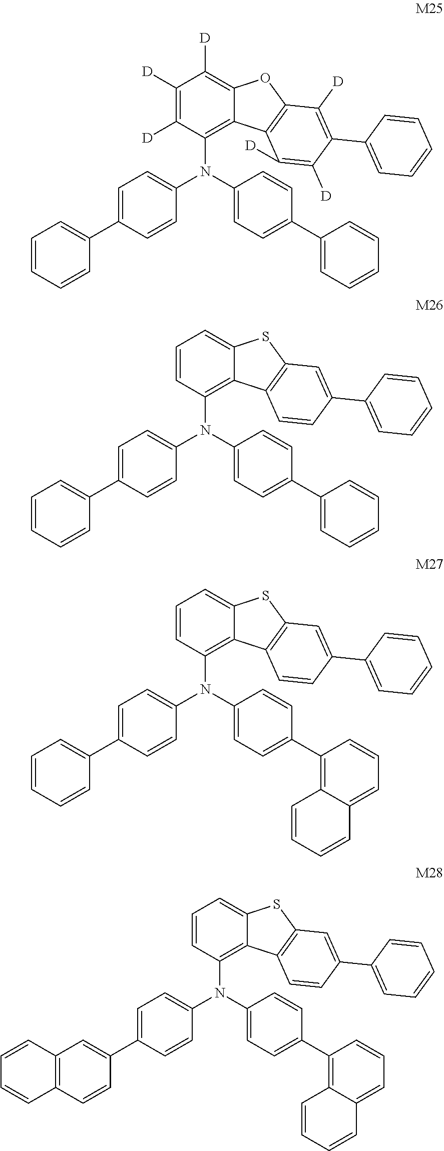 [SMILES: [2H]c1c([2H])c(N(c2ccc(-c3ccccc3)cc2)c2ccc(-c3ccccc3)cc2)c2c(oc3c([2H])c(-c4ccccc4)c([2H])c([2H])c32)c1[2H] ; c1ccc(-c2ccc(N(c3ccc(-c4ccccc4)cc3)c3cccc4sc5cc(-c6ccccc6)ccc5c34)cc2)cc1 ; c1ccc(-c2ccc(N(c3ccc(-c4cccc5ccccc45)cc3)c3cccc4sc5cc(-c6ccccc6)ccc5c34)cc2)cc1 ; c1ccc(-c2ccc3c(c2)sc2cccc(N(c4ccc(-c5ccc6ccccc6c5)cc4)c4ccc(-c5cccc6ccccc56)cc4)c23)cc1] 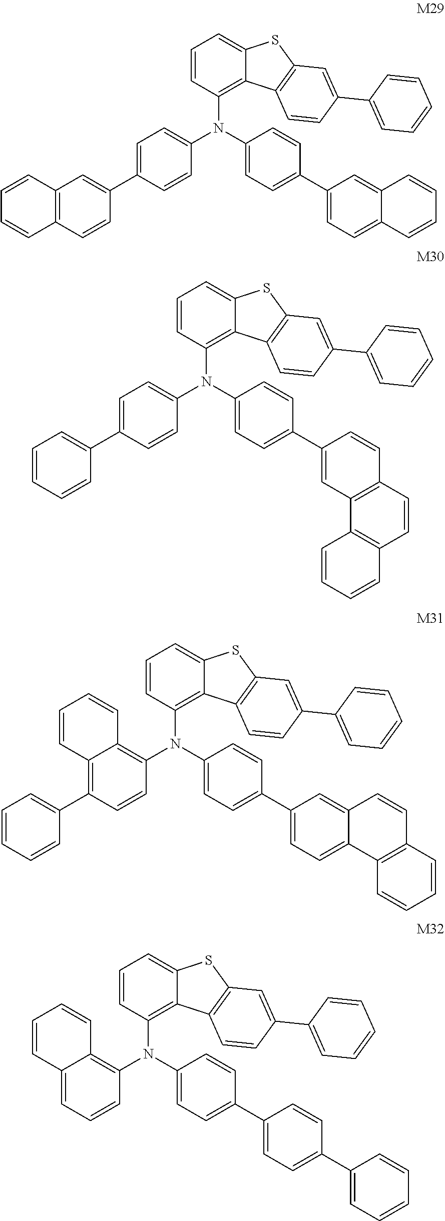 [SMILES: c1ccc(-c2ccc3c(c2)sc2cccc(N(c4ccc(-c5ccc6ccccc6c5)cc4)c4ccc(-c5ccc6ccccc6c5)cc4)c23)cc1 ; c1ccc(-c2ccc(N(c3ccc(-c4ccc5ccc6ccccc6c5c4)cc3)c3cccc4sc5cc(-c6ccccc6)ccc5c34)cc2)cc1 ; c1ccc(-c2ccc3c(c2)sc2cccc(N(c4ccc(-c5ccc6c(ccc7ccccc76)c5)cc4)c4ccc(-c5ccccc5)c5ccccc45)c23)cc1 ; c1ccc(-c2ccc(-c3ccc(N(c4cccc5ccccc45)c4cccc5sc6cc(-c7ccccc7)ccc6c45)cc3)cc2)cc1] 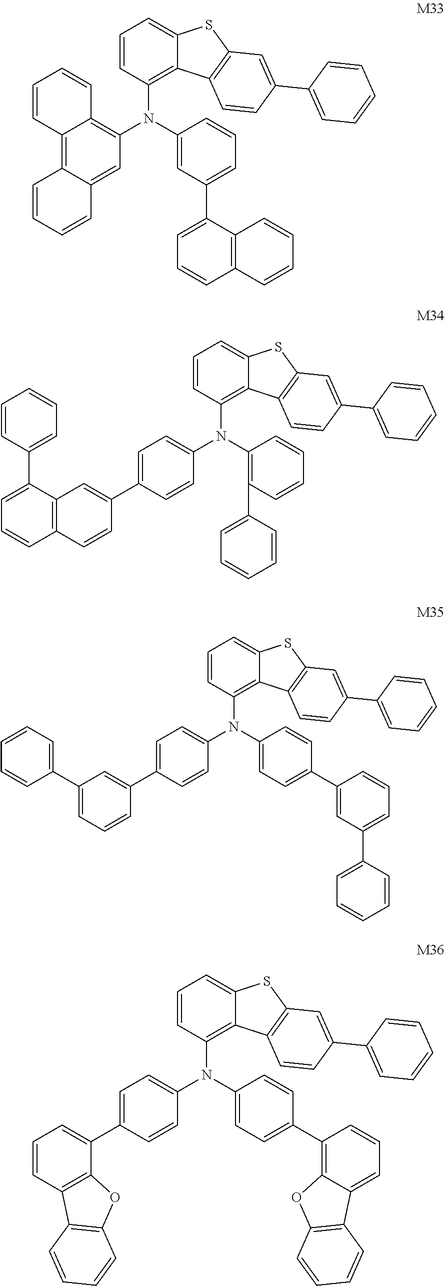 [SMILES: c1ccc(-c2ccc3c(c2)sc2cccc(N(c4cccc(-c5cccc6ccccc56)c4)c4cc5ccccc5c5ccccc45)c23)cc1 ; c1ccc(-c2ccc3c(c2)sc2cccc(N(c4ccc(-c5ccc6cccc(-c7ccccc7)c6c5)cc4)c4ccccc4-c4ccccc4)c23)cc1 ; c1ccc(-c2cccc(-c3ccc(N(c4ccc(-c5cccc(-c6ccccc6)c5)cc4)c4cccc5sc6cc(-c7ccccc7)ccc6c45)cc3)c2)cc1 ; c1ccc(-c2ccc3c(c2)sc2cccc(N(c4ccc(-c5cccc6c5oc5ccccc56)cc4)c4ccc(-c5cccc6c5oc5ccccc56)cc4)c23)cc1] 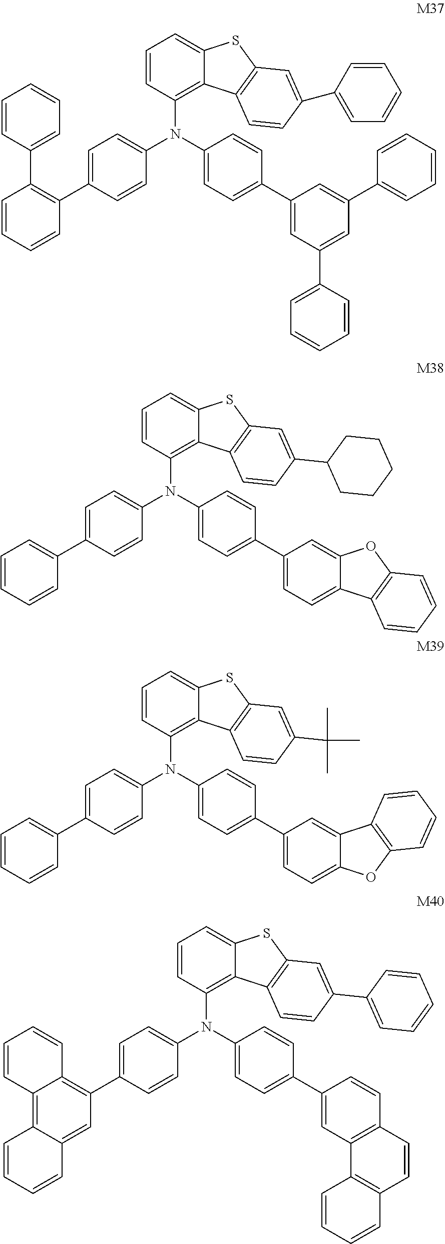 [SMILES: c1ccc(-c2cc(-c3ccccc3)cc(-c3ccc(N(c4ccc(-c5ccccc5-c5ccccc5)cc4)c4cccc5sc6cc(-c7ccccc7)ccc6c45)cc3)c2)cc1 ; c1ccc(-c2ccc(N(c3ccc(-c4ccc5c(c4)oc4ccccc45)cc3)c3cccc4sc5cc(C6CCCCC6)ccc5c34)cc2)cc1 ; CC(C)(C)c1ccc2c(c1)sc1cccc(N(c3ccc(-c4ccccc4)cc3)c3ccc(-c4ccc5oc6ccccc6c5c4)cc3)c12 ; c1ccc(-c2ccc3c(c2)sc2cccc(N(c4ccc(-c5ccc6ccc7ccccc7c6c5)cc4)c4ccc(-c5cc6ccccc6c6ccccc56)cc4)c23)cc1] 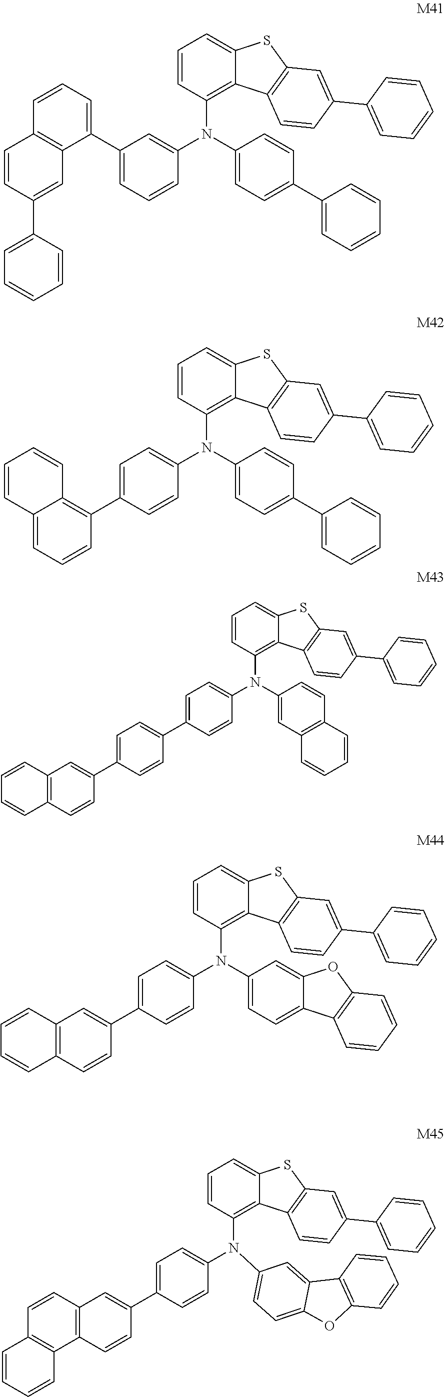 [SMILES: c1ccc(-c2ccc(N(c3cccc(-c4cccc5ccc(-c6ccccc6)cc45)c3)c3cccc4sc5cc(-c6ccccc6)ccc5c34)cc2)cc1 ; c1ccc(-c2ccc(N(c3ccc(-c4cccc5ccccc45)cc3)c3cccc4sc5cc(-c6ccccc6)ccc5c34)cc2)cc1 ; c1ccc(-c2ccc3c(c2)sc2cccc(N(c4ccc(-c5ccc(-c6ccc7ccccc7c6)cc5)cc4)c4ccc5ccccc5c4)c23)cc1 ; c1ccc(-c2ccc3c(c2)sc2cccc(N(c4ccc(-c5ccc6ccccc6c5)cc4)c4ccc5c(c4)oc4ccccc45)c23)cc1 ; c1ccc(-c2ccc3c(c2)sc2cccc(N(c4ccc(-c5ccc6c(ccc7ccccc76)c5)cc4)c4ccc5oc6ccccc6c5c4)c23)cc1] 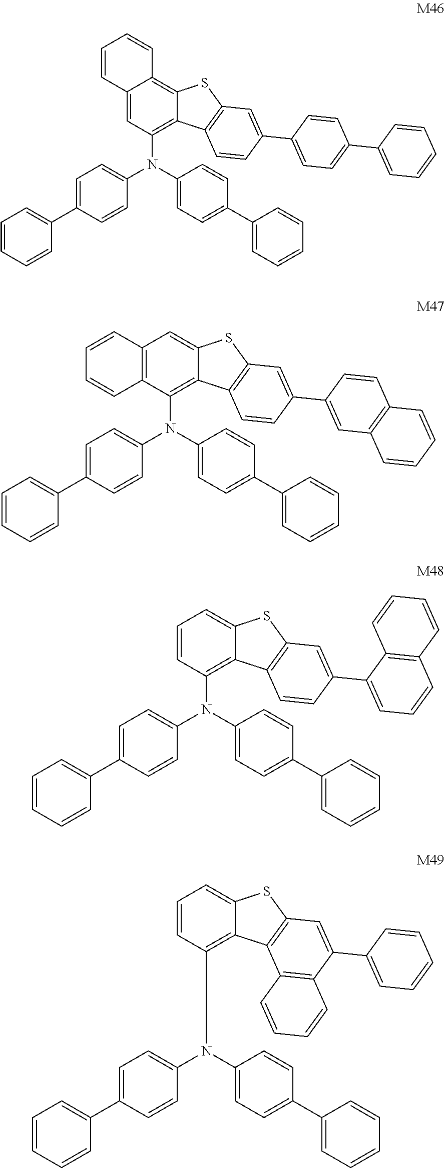 [SMILES: c1ccc(-c2ccc(-c3ccc4c(c3)sc3c5ccccc5cc(N(c5ccc(-c6ccccc6)cc5)c5ccc(-c6ccccc6)cc5)c43)cc2)cc1 ; c1ccc(-c2ccc(N(c3ccc(-c4ccccc4)cc3)c3c4ccccc4cc4sc5cc(-c6ccc7ccccc7c6)ccc5c34)cc2)cc1 ; c1ccc(-c2ccc(N(c3ccc(-c4ccccc4)cc3)c3cccc4sc5cc(-c6cccc7ccccc67)ccc5c34)cc2)cc1 ; c1ccc(-c2ccc(N(c3ccc(-c4ccccc4)cc3)c3cccc4sc5cc(-c6ccccc6)c6ccccc6c5c34)cc2)cc1] 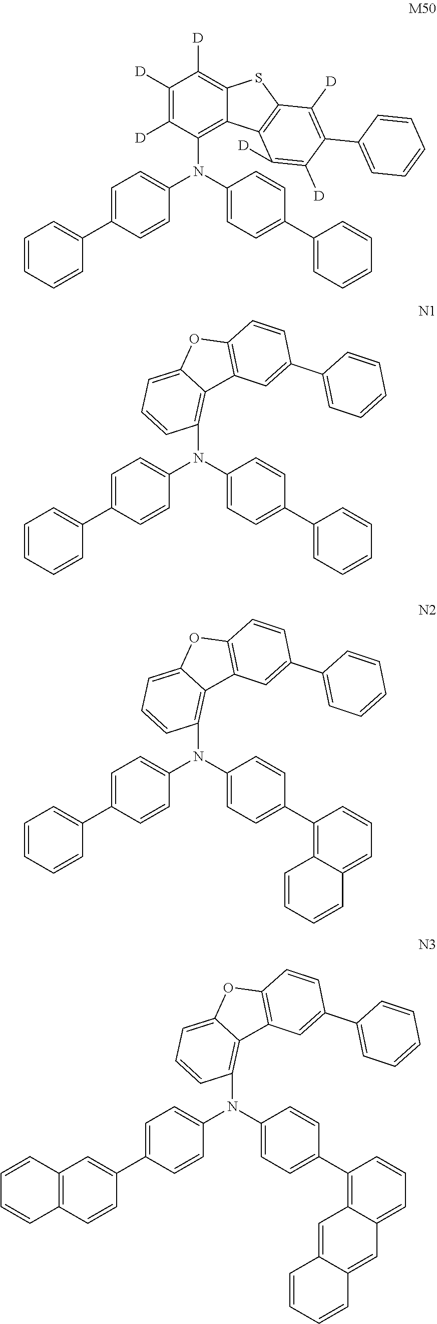 [SMILES: [2H]c1c([2H])c(N(c2ccc(-c3ccccc3)cc2)c2ccc(-c3ccccc3)cc2)c2c(sc3c([2H])c(-c4ccccc4)c([2H])c([2H])c32)c1[2H] ; c1ccc(-c2ccc(N(c3ccc(-c4ccccc4)cc3)c3cccc4oc5ccc(-c6ccccc6)cc5c34)cc2)cc1 ; c1ccc(-c2ccc(N(c3ccc(-c4cccc5ccccc45)cc3)c3cccc4oc5ccc(-c6ccccc6)cc5c34)cc2)cc1 ; c1ccc(-c2ccc3oc4cccc(N(c5ccc(-c6ccc7ccccc7c6)cc5)c5ccc(-c6cccc7cc8ccccc8cc67)cc5)c4c3c2)cc1] 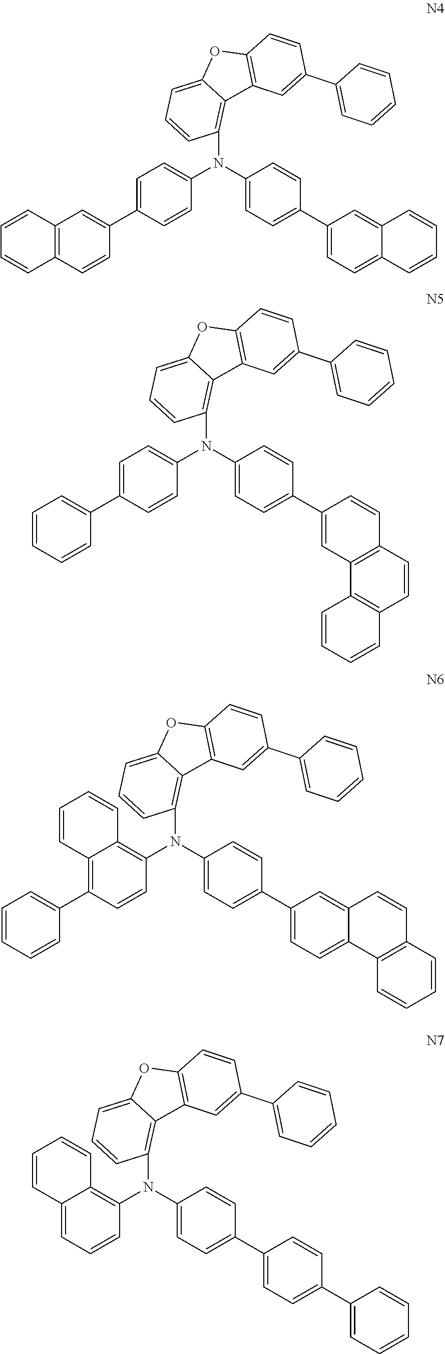 [SMILES: c1ccc(-c2ccc3oc4cccc(N(c5ccc(-c6ccc7ccccc7c6)cc5)c5ccc(-c6ccc7ccccc7c6)cc5)c4c3c2)cc1 ; c1ccc(-c2ccc(N(c3ccc(-c4ccc5ccc6ccccc6c5c4)cc3)c3cccc4oc5ccc(-c6ccccc6)cc5c34)cc2)cc1 ; c1ccc(-c2ccc3oc4cccc(N(c5ccc(-c6ccc7c(ccc8ccccc87)c6)cc5)c5ccc(-c6ccccc6)c6ccccc56)c4c3c2)cc1 ; c1ccc(-c2ccc(-c3ccc(N(c4cccc5ccccc45)c4cccc5oc6ccc(-c7ccccc7)cc6c45)cc3)cc2)cc1] 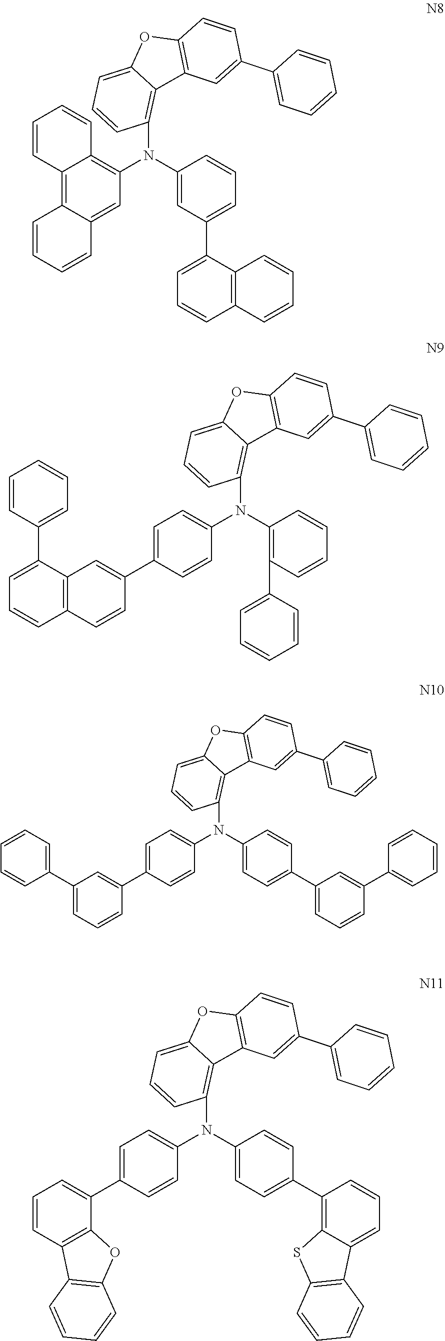 [SMILES: c1ccc(-c2ccc3oc4cccc(N(c5cccc(-c6cccc7ccccc67)c5)c5cc6ccccc6c6ccccc56)c4c3c2)cc1 ; c1ccc(-c2ccc3oc4cccc(N(c5ccc(-c6ccc7cccc(-c8ccccc8)c7c6)cc5)c5ccccc5-c5ccccc5)c4c3c2)cc1 ; c1ccc(-c2cccc(-c3ccc(N(c4ccc(-c5cccc(-c6ccccc6)c5)cc4)c4cccc5oc6ccc(-c7ccccc7)cc6c45)cc3)c2)cc1 ; c1ccc(-c2ccc3oc4cccc(N(c5ccc(-c6cccc7c6oc6ccccc67)cc5)c5ccc(-c6cccc7c6sc6ccccc67)cc5)c4c3c2)cc1] 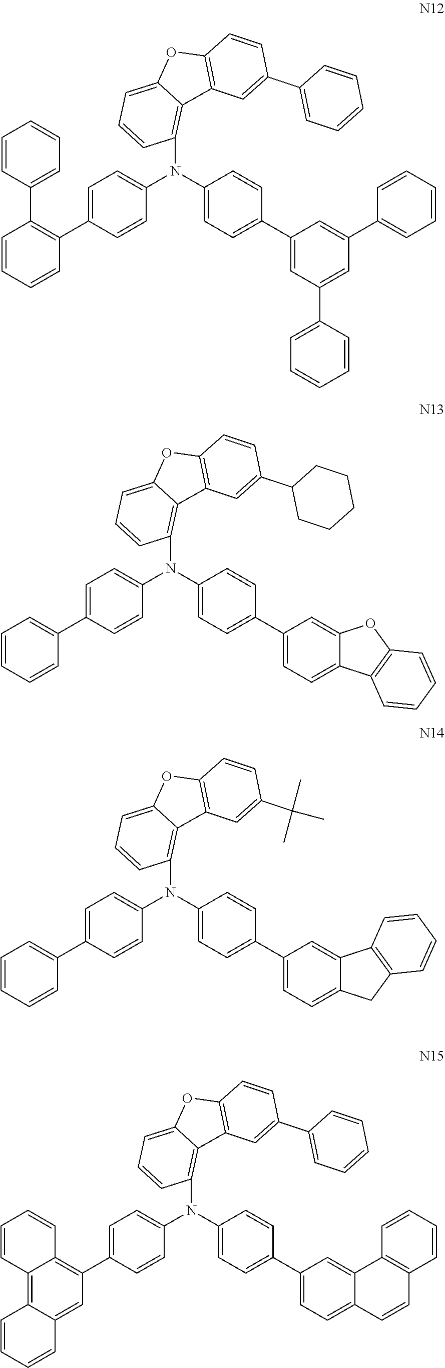 [SMILES: c1ccc(-c2cc(-c3ccccc3)cc(-c3ccc(N(c4ccc(-c5ccccc5-c5ccccc5)cc4)c4cccc5oc6ccc(-c7ccccc7)cc6c45)cc3)c2)cc1 ; c1ccc(-c2ccc(N(c3ccc(-c4ccc5c(c4)oc4ccccc45)cc3)c3cccc4oc5ccc(C6CCCCC6)cc5c34)cc2)cc1 ; CC(C)(C)c1ccc2oc3cccc(N(c4ccc(-c5ccccc5)cc4)c4ccc(-c5ccc6c(c5)-c5ccccc5C6)cc4)c3c2c1 ; c1ccc(-c2ccc3oc4cccc(N(c5ccc(-c6ccc7ccc8ccccc8c7c6)cc5)c5ccc(-c6cc7ccccc7c7ccccc67)cc5)c4c3c2)cc1] 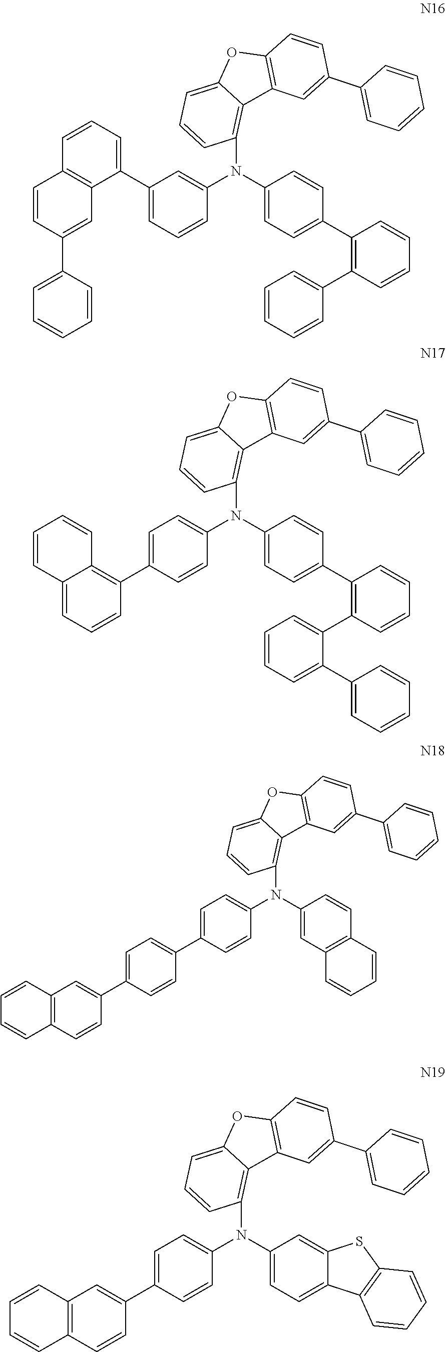 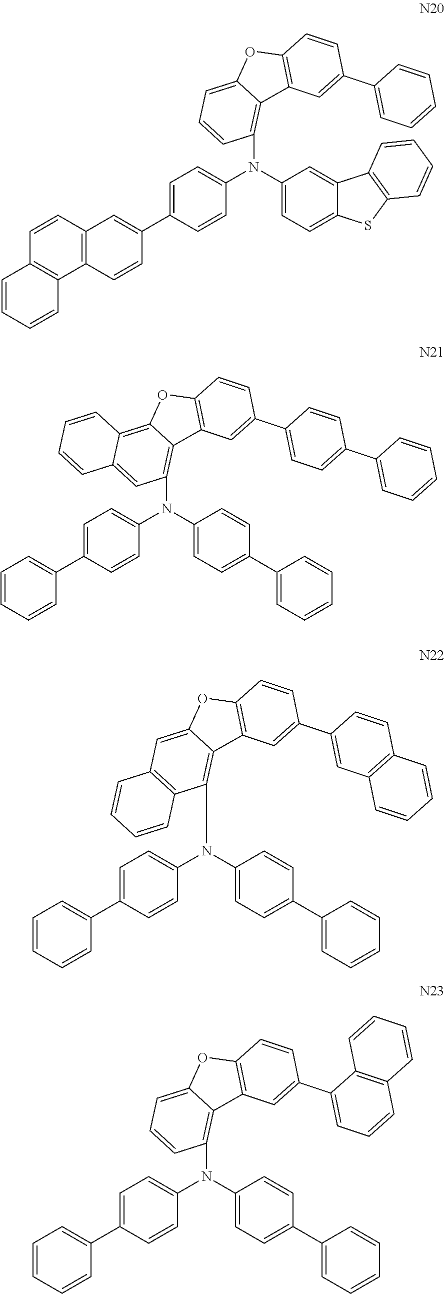 [SMILES: c1ccc(-c2ccc3oc4cccc(N(c5ccc(-c6ccc7c(ccc8ccccc87)c6)cc5)c5ccc6sc7ccccc7c6c5)c4c3c2)cc1 ; c1ccc(-c2ccc(-c3ccc4oc5c6ccccc6cc(N(c6ccc(-c7ccccc7)cc6)c6ccc(-c7ccccc7)cc6)c5c4c3)cc2)cc1 ; c1ccc(-c2ccc(N(c3ccc(-c4ccccc4)cc3)c3c4ccccc4cc4oc5ccc(-c6ccc7ccccc7c6)cc5c34)cc2)cc1 ; c1ccc(-c2ccc(N(c3ccc(-c4ccccc4)cc3)c3cccc4oc5ccc(-c6cccc7ccccc67)cc5c34)cc2)cc1] 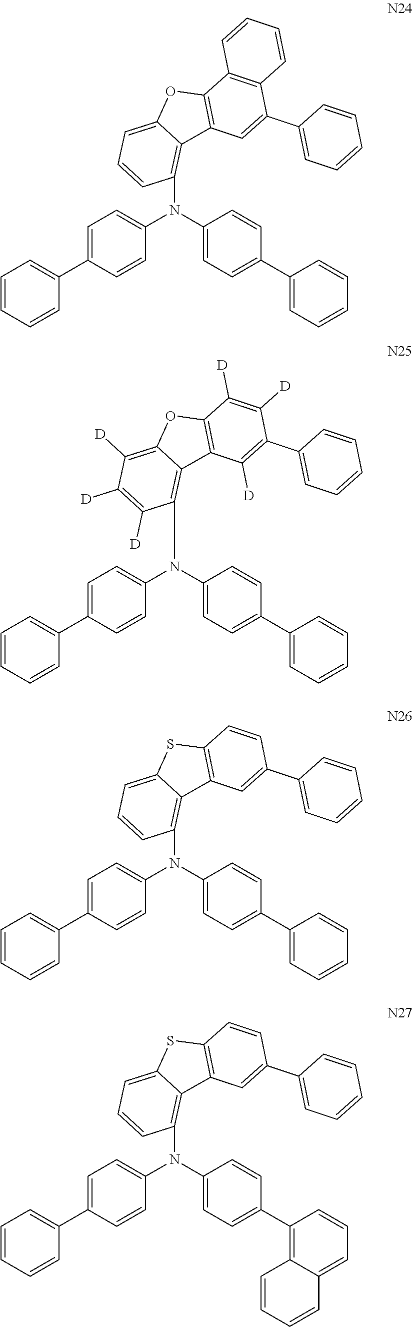 [SMILES: c1ccc(-c2ccc(N(c3ccc(-c4ccccc4)cc3)c3cccc4oc5c6ccccc6c(-c6ccccc6)cc5c34)cc2)cc1 ; [2H]c1c(-c2ccccc2)c([2H])c2c(oc3c([2H])c([2H])c([2H])c(N(c4ccc(-c5ccccc5)cc4)c4ccc(-c5ccccc5)cc4)c32)c1[2H] ; c1ccc(-c2ccc(N(c3ccc(-c4ccccc4)cc3)c3cccc4sc5ccc(-c6ccccc6)cc5c34)cc2)cc1 ; c1ccc(-c2ccc(N(c3ccc(-c4cccc5ccccc45)cc3)c3cccc4sc5ccc(-c6ccccc6)cc5c34)cc2)cc1] 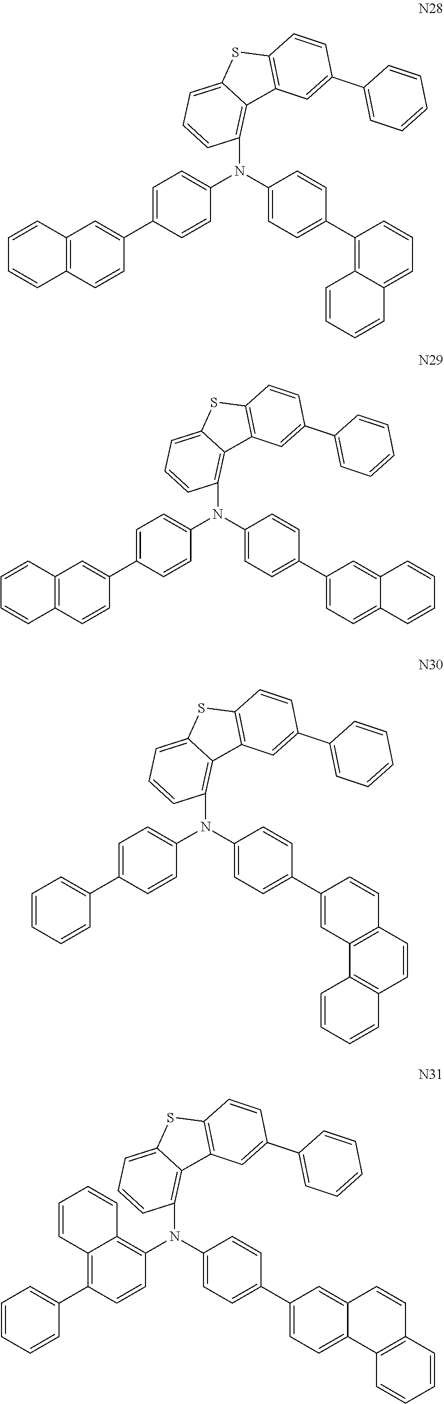 [SMILES: c1ccc(-c2ccc3sc4cccc(N(c5ccc(-c6ccc7ccccc7c6)cc5)c5ccc(-c6cccc7ccccc67)cc5)c4c3c2)cc1 ; c1ccc(-c2ccc3sc4cccc(N(c5ccc(-c6ccc7ccccc7c6)cc5)c5ccc(-c6ccc7ccccc7c6)cc5)c4c3c2)cc1 ; c1ccc(-c2ccc(N(c3ccc(-c4ccc5ccc6ccccc6c5c4)cc3)c3cccc4sc5ccc(-c6ccccc6)cc5c34)cc2)cc1 ; c1ccc(-c2ccc3sc4cccc(N(c5ccc(-c6ccc7c(ccc8ccccc87)c6)cc5)c5ccc(-c6ccccc6)c6ccccc56)c4c3c2)cc1] 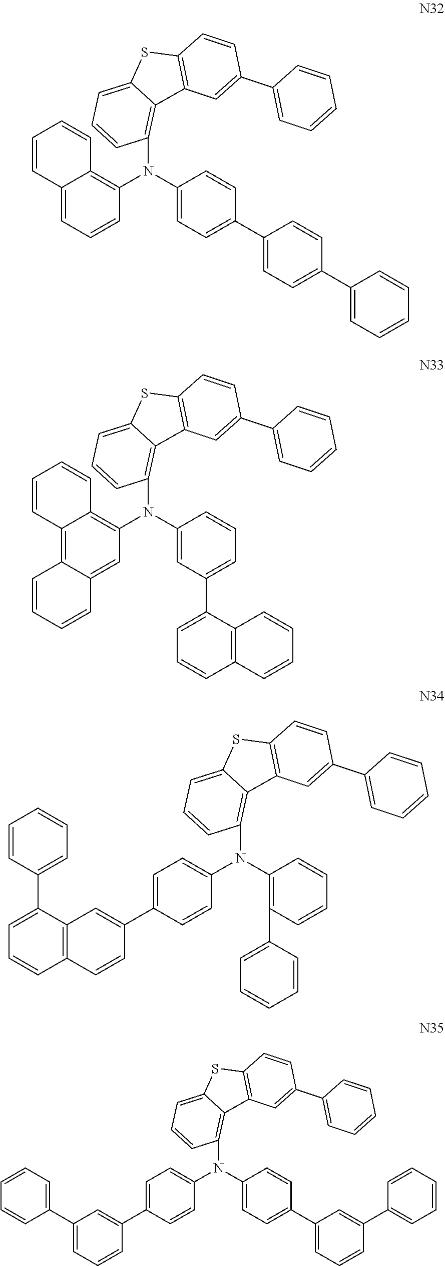 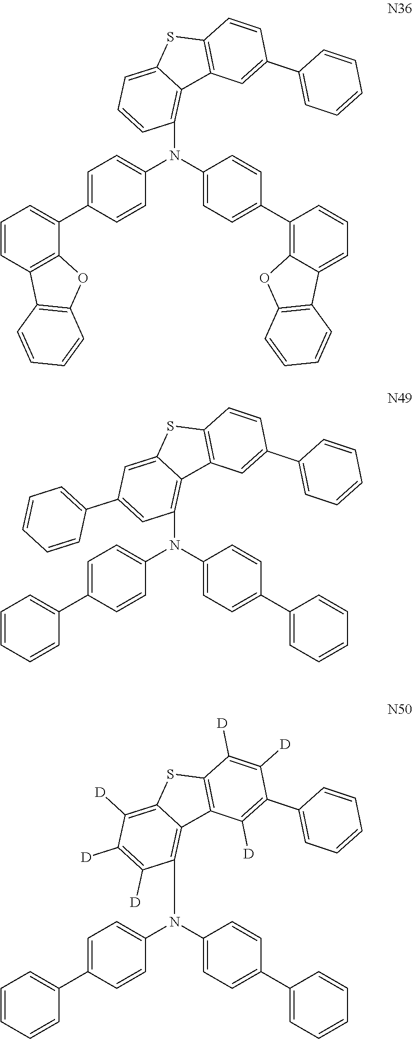 [SMILES: c1ccc(-c2ccc3sc4cccc(N(c5ccc(-c6cccc7c6oc6ccccc67)cc5)c5ccc(-c6cccc7c6oc6ccccc67)cc5)c4c3c2)cc1 ; c1ccc(-c2ccc(N(c3ccc(-c4ccccc4)cc3)c3cc(-c4ccccc4)cc4sc5ccc(-c6ccccc6)cc5c34)cc2)cc1 ; [2H]c1c(-c2ccccc2)c([2H])c2c(sc3c([2H])c([2H])c([2H])c(N(c4ccc(-c5ccccc5)cc4)c4ccc(-c5ccccc5)cc4)c32)c1[2H]]
